(19) United States
(12) Patent Application Publication
(10) Pub. No.: US 2025/0214991 A1
(43) Pub. Date: Jul. 3, 2025

(54) BICYCLIC HETEROARYL-CONTAINING COMPOUNDS AS IKZF2 DEGRADERS

(71) Applicant: Oncopia Therapeutics, Inc. d/b/a/ SK Life Science Labs, King Of Prussia, PA (US)

(72) Inventors: Qiaolin DENG, King of Prussia, PA (US); Xuqing ZHANG, King of Prussia, PA (US)

(21) Appl. No.: 18/850,016

(22) PCT Filed: Mar. 24, 2023
(86) PCT No.: PCT/US2023/064930

§ 371 Date: Sep. 23, 2024

Related U.S. Application Data
(60) Provisional application No. 63/323,656, filed on Mar. 25, 2022.

(51) Int. Cl. C07D 471/04; A61K 31/4545; A61K 31/4709; A61K 31/4725; A61K 31/496; A61K 31/4985; A61K 31/519; A61K 31/5377; A61K 31/541; A61K 31/55; A61K 31/551; A61P 35/00; C07D 401/14; C07D 487/04; C07D 498/04; C07D 519/00

(52) U.S. Cl. CPC C07D 471/04; A61K 31/4545; A61K 31/4709; A61K 31/4725; A61K 31/496; A61K 31/4985; A61K 31/519; A61K 31/5377; A61K 31/541; A61K 31/55; A61K 31/551; A61P 35/00; C07D 401/14; C07D 487/04; C07D 498/04; C07D 519/00

(57) ABSTRACT

Described herein are compounds of Formula II and pharmaceutically acceptable salts, solvates, or stereoisomers thereof, as well as their uses (e.g., as IKZF2 degraders) in treating or preventing diseases or disorders.

### RELATED APPLICATIONS

This application claims the benefit of and priority to U.S. Provisional Application No. 63/323,656, filed Mar. 25, 2022, the contents of which are incorporated herein by reference in their entireties.

### BACKGROUND

IKAROS Family Zinc Finger 2 (IKZF2) (also known as Helios) is one of the five members of the Ikaros family of transcription factors found in mammals. IKZF2 contains four zinc finger domains near the N-terminus, which are involved in DNA binding, and two zinc finger domains at the C-terminus, which are involved in protein dimerization. IKZF2 is about 50% identical with Ikaros family members, Ikaros (IKZF1), Aiolos (IKZF3), and Eos (IKZF4) with highest homology in the zinc finger regions (80%+ identity). These four Ikaros family transcription factors bind to the same DNA consensus site and can heterodimerize with each other when co-expressed in cells. The fifth Ikaros family protein, Pegasus (IKZF5), is only 25% identical to IKZF2, binds a different DNA site than other Ikaros family members and does not readily heterodimerize with the other Ikaros family proteins. IKZF2, IKZF1 and IKZF3 are expressed mainly in hematopoietic cells while IKZF4 and IKZF5 are expressed in a wide variety of tissues.

IKZF2 is a critical regulator of T cell activity and function. Genetic deletion of Helios resulted in an enhanced anti-tumor immune response. Notably, Helios is highly expressed in regulatory T cells, a subpopulation of T cells that restricts the activity of effector T cells. Selective deletion of Helios in regulatory T cells resulted in both loss of suppressive activity and acquisition of effector T cell functions. Therefore, Helios is a critical factor in restricting T cell effector function in Tregs. Currently, anti-CTLA4 antibodies are used in the clinic to target Tregs in tumors. However, targeting CTLA4 often causes systemic activation of T-effector cells, resulting in excessive toxicity and limiting therapeutic utility. Up to 3/4 of patients treated with a combination of anti-PD-1 and anti-CTLA4 have reported grade 3 or higher adverse events. Thus, a strong need exists to provide compounds that target Tregs in tumors without causing systemic activation of T-effector cells. An IKZF2-specific degrader has the potential to focus the enhanced immune response to areas within or near tumors providing a potentially more tolerable and less toxic therapeutic agent for the treatment of cancer.

Helios expression has also been reported to be upregulated in ‘exhausted’ T cells, in the settings of both chronic viral infections, as well as in dysfunctional chimeric antigen receptor (CAR) T cells. Overexpression or aberrant expression of Helios and various splice isoforms have been reported in several hematological malignancies, including T cell leukemias and lymphomas. Moreover, knockdown of Helios in a model of mixed lineage leukemia (MLL)-driven myeloid leukemia potently suppressed proliferation and increased cell death. In line with these results, genomic profiling and chromatin accessibility analysis demonstrated that IKZF2 loss led to increased myeloid differentiation. These data suggest that IKZF2 is differentially required in myeloid leukemia cells compared to normal cells. Therefore, depletion of IKZF2 has preferential effect in leukemic stem cells compared to normal hematopoietic stem cells, providing a new strategy for targeting leukemic stem cells.

### SUMMARY

In certain aspects, the present disclosure provides compounds of Formula II:

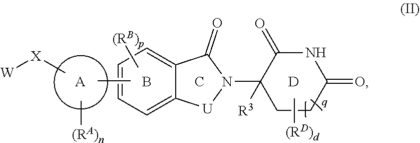

and pharmaceutically acceptable salts, solvates, or stereoisomers thereof, wherein each of the variables in Formula II, is described, embodied, and exemplified herein.

In certain aspects, the present disclosure provides pharmaceutical compositions comprising a compound disclosed herein, and a pharmaceutically acceptable excipient.

In certain aspects, the present disclosure further provides methods of degrading an IKZF2 protein in a subject or biological sample comprising administering a compound disclosed herein to the subject or contacting the biological sample with a compound disclosed herein.

In certain aspects, the present disclosure further provides uses of a compound disclosed herein in the manufacture of a medicament for degrading an IKZF2 protein in a subject or biological sample.

In certain aspects, the present disclosure provides compounds disclosed herein for use in degrading an IKZF2 protein in a subject or biological sample.

In certain aspects, the present disclosure provides methods of treating an IKZF2-mediated disease or disorder comprising administering to a subject in need thereof a compound disclosed herein.

In certain aspects, the present disclosure provides uses of a compound disclosed herein in the manufacture of a medicament for treating an IKZF2-mediated disease or disorder.

In certain aspects, the present disclosure provides compounds disclosed herein for use in treating an IKZF2-mediated disease or disorder.

In certain aspects, the present disclosure provides methods of (a) increasing IL-2 production; (b) suppressing regulatory T cells; (c) enhancing effector T cells; (d) inhibiting tumor growth; and/or (e) enhancing tumor regression in a subject, comprising administering to the subject in need thereof a compound disclosed herein.

In certain aspects, the present disclosure provides uses of a compound disclosed herein in the manufacture of a medicament for (a) increasing IL-2 production; (b) suppressing regulatory T cells; (c) enhancing effector T cells; (d) inhibiting tumor growth; and/or (e) enhancing tumor regression in a subject.

### DETAILED DESCRIPTION

The present disclosure relates to compounds and methods of degrading a IKZF2 protein comprising contacting a IKZF2 protein with a IKZF2 degrader. The invention also relates to methods of treating a IKZF2 protein-mediated disease or disorder in a patient by administering a IKZF2 degrader to a patient in need thereof. The invention further relates to methods of treating a IKZF2-mediated disease or disorder in a patient, the method comprising administering a pharmaceutical composition comprising a IKZF2 degrader to a patient in need thereof.

### Compounds of the Present Disclosure

The present disclosure provides compounds of Formula II:

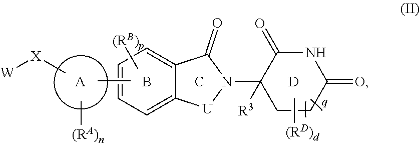

and pharmaceutically acceptable salts, solvates, or stereoisomers thereof, wherein:
W is —N(R¹)₂, 3- to 12-membered heterocyclyl, or 5- to 10-membered heteroaryl, wherein the heterocyclyl or heteroaryl is optionally substituted with one or more R^{1b};
two R¹, together with the nitrogen atom to which they are attached, form 3- to 12-membered heterocyclyl or 5- to 10-membered heteroaryl, wherein the heterocyclyl or heteroaryl is optionally substituted with one or more R^{1b}.
each R^{1b} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O) R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c} R^{d}, —C(—O)R^{a}, —C(═O)OR^{b}, or —C(—O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
two vincinal R^{1b}, together with the intervening atoms, form C₆₋₁₀ aryl or 5- to 10-membered heteroaryl, wherein the aryl or heteroaryl is optionally substituted with one or more R^{u}; or
each R¹ is independently hydrogen, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkylene, alkenyl, alkynyl, carbocyclyl, heterocyclyl, aryl, or heteroaryl is optionally substituted with one or more R^{1a},
each R^{1a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c} S(═O) R^{a}, —NR^{c} S(═O)₂OR^{b}, —NR^{c}S(═O)₂NR^{c} R^{a}, —NR^{b}C(═O)NR^{c}R^{d}, —NR^{b}C(═O) R^{a}, —NR^{b}C(—O)OR^{b}, —OS(═O)₂R^{a}, —OS(═O)₂OR^{b}, —OS(═O)₂NR^{c}R^{d}, —OC(═O)R^{a}, —OC(—O)OR^{b}, —OC(—O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
X is —[C(R²)₂]—ₘ, O, or NR^{X}, wherein when X is O or NR^{X}, then W is 3- to 12-membered heterocyclyl or 5- to 10-membered heteroaryl, wherein the heterocyclyl or heteroaryl is optionally substituted with one or more R^{1b},
each R² is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(—O)R^{a}, —S(—O)₂R^{a}, —S(═O)₂OR^{b}, —S(═O)₂NR^{c}R^{d}, —NR^{c}S(═O)₂R^{a}, —NR^{c}S(═O) R^{a}, —NR^{c} S(—O)₂OR^{b}, —NR^{c}S(═O)₂NR^{c}R^{d}, —NR^{b}C(═O)NR^{c}R^{d}, —NR^{b}C(═O) R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c} R⁴, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two geminal R² together form an oxo; or
two germinal R², together with the carbon atom to which they are attached, form C₃₋₆ carbocyclyl or 3- to 6-membered heterocyclyl, wherein the carbocyclyl or heterocyclyl is optionally substituted with one or more R^{u};
m is an integer from 0 to 5;
R^{X} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
Ring A is 9- or 10-membered bicyclic fused ring system comprising at least one 5- or 6-membered heteoaryl;
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(—O)₂R^{a}, —S(═O)₂OR^{b}, —S(═O)₂NR^{c}R^{d}, —NR^{c} S(═O)₂R^{a}, —NR^{c} S(═O) R^{a}, —NR^{c} S(═O)₂OR^{b}, —NR^{c}S(═O)₂NR^{c}R^{d}, —NR^{b}C(═O)NR^{c} R^{a}, —NR^{b}C(O) R^{a}, —NR^{c}C(—O)OR^{d}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
n is an integer from 0 to 10, as valency permits; or
two vincinal R^{A}, together with the intervening atoms, form C₃₋₁₂ carbocyclyl or 3- to 12-membered heterocyclyl, wherein the carbocyclyl or heterocyclyl is optionally substituted with one or more R^{u};
each R^{B} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c} S(═O)₂R^{a}, —NR^{c} S(═O) R^{a}, —NR^{c}S(═O)₂OR^{b}, —NR^{c}S(═O)₂NR^{c}R^{d}, —NR^{b}C(═O)NR^{c}R^{d}, —NR^{b}C(═O) R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(—O) R^{a}, —C(═O)OR^{b}, or —C(—O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
p is an integer from 0 to 3;
U is —C(R⁴)₂— or —C(═O)—;
each R⁴ is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}; or
two R⁴, together with the carbon atom to which they are attached, form C₃₋₆ carbocyclyl or 3- to 6-membered heterocyclyl, wherein the carbocyclyl or heterocyclyl is optionally substituted with one or more R^{u};
each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 4;
R³ is hydrogen, deuterium, C₁₋₆ haloalkyl, or C₁₋₆ alkyl; and
q is an integer from 0 to 2;
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(—O)₂OR^{b}, —S(═O)₂NR^{c} R⁴, —NR^{c} S(═O)₂R^{a}, —NR^{c} S(═O) R^{a}, —NR^{c} S(═O)₂OR^{b}, —NR^{c}S(═O)₂NR^{c}R^{d}, —NR^{b}C(═O)NR^{c}R^{d}, —NR^{b}C(═O) R^{a}, —NR^{b}C(═O)OR^{b}, —OS(═O)₂R^{a}, —OS(═O)₂OR^{b}, —OS(═O)₂NR^{c}R^{d}, —OC(—O)R^{a}, —OC(═O)OR^{b}, —OC(═O)NR^{c}R^{d}, —C(═O) R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino,
C₃₋₆ carbocyclyl, 3- to 6-membered heterocyclyl, C₆ aryl, and 5- to 6-membered heteroaryl; or
two R^{u}, together with the one or more intervening atoms, form C₆₋₁₀ aryl, 5- to 10-membered heteroaryl, C₃₋₁₂ carbocyclyl, or 3- to 12-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl or 5- to 10-membered heteroaryl, wherein the heterocyclyl or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₃₋₆ carbocyclyl, and 3- to 6-membered heterocyclyl;
wherein each of R^{a}, R^{b}, R^{c}, and R^{d} is independently and optionally substituted with one or more R^{z};
each R^{z} is independently oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-memberred heterocyclyl, C₆ aryl, or 5- to 6-membered heteroaryl.

In certain embodiments, when Ring A is 10-membered bicyclic heteroaryl, Ring A is not isoquinolinyl.

In certain embodiments,
i) when Ring A is

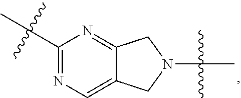 [SMILES: *c1ncc2c(n1)CN(*)C2]

then m is not 0;
ii) when each R¹ is independently hydrogen, C₁₋₆ alkyl, C₃₋₁₂ carbocyclyl, or —C(═O) (C₁₋₆ alkyl), then 1) m is not 0; and 2) two geminal R² do not together form an oxo; and
iii) the compound is not

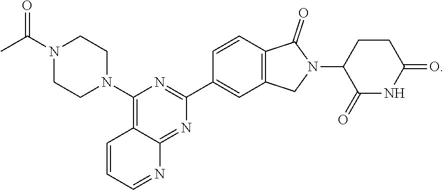 [SMILES: CC(=O)N1CCN(c2nc(-c3ccc4c(c3)CN(C3CCC(=O)NC3=O)C4=O)nc3ncccc23)CC1]

In certain embodiments, Ring A is not pyrido[2,3-d]pyrimidinyl.

In certain embodiments, two R¹, together with the nitrogen atom to which they are attached, form piperazinyl optionally substituted with one or more R^{1b}, then Ring A is not pyrido[2,3-d]pyrimidinyl.

In certain embodiments, the compound is a compound of Formula II-1

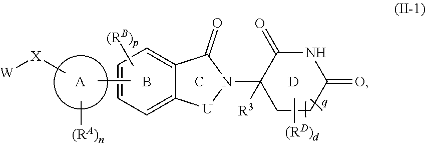

or a pharmaceutically acceptable salt, solvate, or stereoisomer thereof.

In certain embodiments, the compound is a compound of Formula II-2

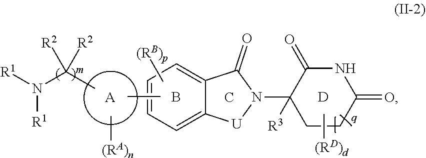

or a pharmaceutically acceptable salt, solvate, or stereoisomer thereof.

In certain embodiments, W is —N(R¹)₂, 3- to 12-membered heterocyclyl (e.g., heterocyclyl comprising one or two 3- to 8-membered rings and 1-5 heteroatoms selected from N, O, and S), or 5- to 10-membered heteroaryl (e.g., heteroaryl comprising one or two 5- or 6-membered rings and 1-5 heteroatoms selected from N, O, and S), wherein the heterocyclyl or heteroaryl is optionally substituted with one or more R^{1b}.

In certain embodiments, two R¹, together with the nitrogen atom to which they are attached, form 3- to 12-membered heterocyclyl (e.g., heterocyclyl comprising one or two 3- to 8-membered rings and 1-5 heteroatoms selected from N, O, and S) or 5- to 10-membered heteroaryl (e.g., heteroaryl comprising one or two 5- or 6-membered rings and 1-5 heteroatoms selected from N, O, and S), wherein the heterocyclyl or heteroaryl is optionally substituted with one or more R^{1b}.

In certain embodiments, each R¹ is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —S(═O)₂R^{a}, —S(═O)₂OR^{b}, —S(═O)₂NR^{c}R^{d}, —C(═O) R^{a}, —C(—O)OR^{b}, or —C(═O)NR^{c}R^{d}, wherein the alkyl, alkylene, alkenyl, alkynyl, carbocyclyl, heterocyclyl, aryl, or heteroaryl is optionally substituted with one or more R^{1a}.

In certain embodiments, each R¹ is independently hydrogen, C₁₋₆ alkyl, —(C₁₋₆ alkylene)-(C₆₋₁₀ aryl), or —(C₁₋₆ alkylene)-(5- to 10-membered heteroaryl), wherein the alkyl, alkylene, aryl, or heteroaryl is optionally substituted with one or more R^{1a}.

In certain embodiments, each R^{1a} is independently oxo, halogen (e.g., —F, —C₁, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), n-propoxy (C₃), i-propoxy (C₃), n-butoxy (C₄), i-butoxy (C₄), S-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, S-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c} S(═O) R^{a}, —NR^{c} S(—O)₂OR^{b}, —NR^{c}S(═O)₂NR^{c} R^{a}, —NR^{b}C(═O)NR^{c} R⁴, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{1a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{1a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{1a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl, is optionally substituted with one or more R^{u}.

In certain embodiments, each R^{1a} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl, is optionally substituted with one or more R^{u}.

In certain embodiments, each R^{1a} is independently halogen, C₁₋₆ alkyl, C₆₋₁₀ aryl, or 5- to 10-membered heteroaryl, wherein the alkyl, aryl, or heteroaryl is optionally substituted with one or more R^{u}.

In certain embodiments, each R^{1b} is independently oxo, halogen (e.g., —F, —C₁, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), n-propoxy (C₃), i-propoxy (C₃), n-butoxy (C₄), i-butoxy (C₄), S-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, S-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c} S(═O) R^{a}, —NR^{c} S(═O)₂OR^{b}, —NR^{c}S(═O)₂NR^{c} R⁴, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(—O)R^{a}, —C(—O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{1b} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{1b} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{1b} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl, is optionally substituted with one or more R^{u}.

In certain embodiments, each R^{1b} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl, is optionally substituted with one or more R^{u}.

In certain embodiments, each R^{1b} is independently oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, C₆ aryl, 5- to 6-membered heteroaryl, or —S(═O)₂R^{a}, wherein the alkyl, alkoxy, alkylamino, carbocyclyl, or heterocyclyl is optionally substituted with one or more R^{u}.

In certain embodiments, each R^{1b} is independently oxo, halogen, —CN, —OH, C₁₋₆ alkyl, C₁₋₆ alkoxy, C₁₋₆ alkylamino, C₆ aryl, 5- to 6-membered heteroaryl, or —S(═O)₂R^{a}, wherein the alkyl, alkoxy, alkylamino, aryl, or heteroaryl is optionally substituted with one or more R^{u}.

In certain embodiments, two vincinal R^{1b}, together with the intervening atoms, form C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), wherein the aryl or heteroaryl is optionally substituted with one or more R^{u}.

In certain embodiments, X is —[C(R²)₂]-ₘ, O, or NR^{X}.

In certain embodiments, X is —[C(R²)₂]-ₘ. In certain embodiments, X is O. In certain embodiments, X is NR^{X}. In certain embodiments, when X is O or NR^{X}, then W is 3- to 12-membered heterocyclyl or 5- to 10-membered heteroaryl, wherein the heterocyclyl or heteroaryl is optionally substituted with one or more R^{1b}.

In certain embodiments, each R² is independently hydrogen, halogen (e.g., —F, —C₁, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), n-propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, S-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c} S(═O) R^{a}, —NR^{c} S(═O)₂OR^{b}, —NR^{c}S(═O)₂NR^{c} R⁴, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c} R⁴, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R² is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R² is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R² is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl, is optionally substituted with one or more R^{u}.

In certain embodiments, each R² is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl, is optionally substituted with one or more R^{u}.

In certain embodiments, each R² is independently hydrogen or C₁₋₆ alkyl. In certain embodiments, each R² is hydrogen.

In certain embodiments, two geminal R² together form an oxo.

In certain embodiments, two germinal R², together with the carbon atom to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S), wherein the carbocyclyl or heterocyclyl is optionally substituted with one or more R^{u}.

In certain embodiments, m is an integer from 0 to 5. In certain embodiments, m is 0. In certain embodiments, m is 1. In certain embodiments, m is 2. In certain embodiments, m is 3. In certain embodiments, m is 4. In certain embodiments, m is 5.

In certain embodiments, R^{X} is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (CA), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R^{X} is hydrogen, C₁₋₆ alkyl, C₂₋₆ alkenyl, C₂₋₆ alkynyl, C₃₋₆ carbocyclyl, 3- to 6-membered heterocyclyl, C₆ aryl, 5- to 6-membered heteroaryl, —S(═O)₂R^{a}, —S(═O)₂OR^{b}, —S(═O)₂NR^{c} R⁴, —C(—O) R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R^{X} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O) R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, Ring A is 9- or 10-membered bicyclic fused ring system comprising at least one 5- or 6-membered heteoaryl (e.g., heteroaryl comprising one 5- or 6-membered ring and 1-4 heteroatoms selected from N, O, and S).

In certain embodiments, Ring A is 9- or 10-membered bicyclic fused ring system comprising one 5- or 6-membered heteoaryl (e.g., heteroaryl comprising one 5- or 6-membered ring and 1-4 heteroatoms selected from N, O, and S) and one C₅₋₆ carbocyclyl (e.g., cyclopentyl (C₅), cyclopentenyl (C₅), cyclohexyl (C₆), cyclohexenyl (C₆), or cyclohexadienyl (C₆)) or 5- to 6-membered heterocyclyl (e.g., heterocyclyl comprising one 5- to 6-membered ring and 1-3 heteroatoms selected from N, O, and S).

In certain embodiments, Ring A is 9- or 10-membered bicyclic heteroaryl (e.g., bicyclic heteroaryl comprising two 5- or 6-membered rings and 1-5 heteroatoms selected from N, O, and S).

In certain embodiments, Ring A is 9-membered bicyclic heteroaryl (e.g., bicyclic heteroaryl comprising one 5-membered ring and one 6-membered ring, and 1-5 heteroatoms selected from N, O, and S, wherein at least one of the 5-membered ring and the 6-membered ring is heteoaryl).

In certain embodiments, Ring A is 9-membered bicyclic heteroaryl comprising 1 to 4 nitrogen atoms.

In certain embodiments, Ring A is imidazo[1,5-a]pyridinyl, 1H-pyrrolo[2,3-b]pyridinyl, [1,2,4]triazolo[4,3-a]pyridinyl, imidazo[1,2-a]pyridinyl, indolyl, benzo[d]imidazolyl, indazolyl, benzo[d]isoxazolyl, benzo[d]oxazolyl, benzo[d]isothiazolyl, benzo[d]thiazolyl, benzo[b]thiophenyl, or benzofuranyl.

In certain embodiments,

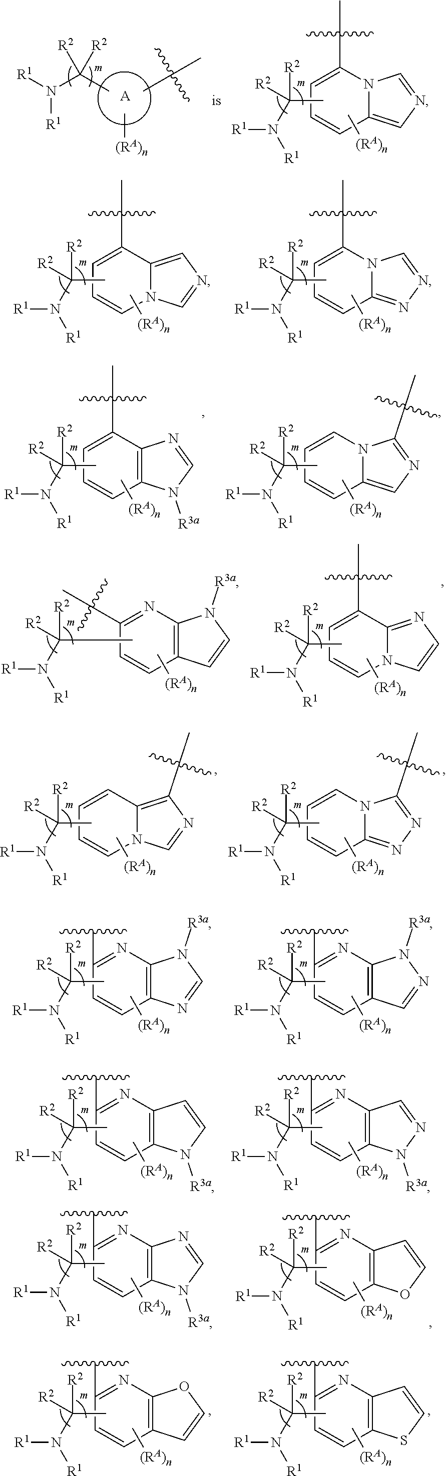

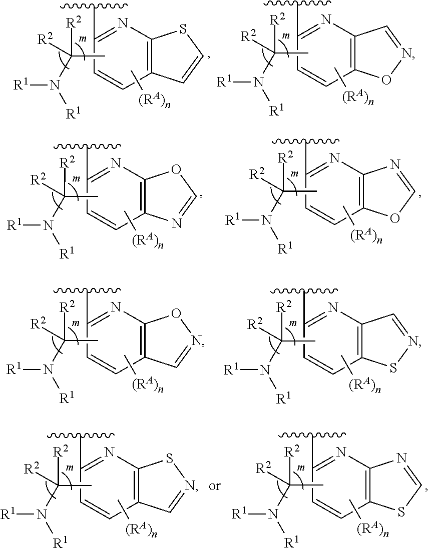

wherein:
R^{3a} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(—O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments,

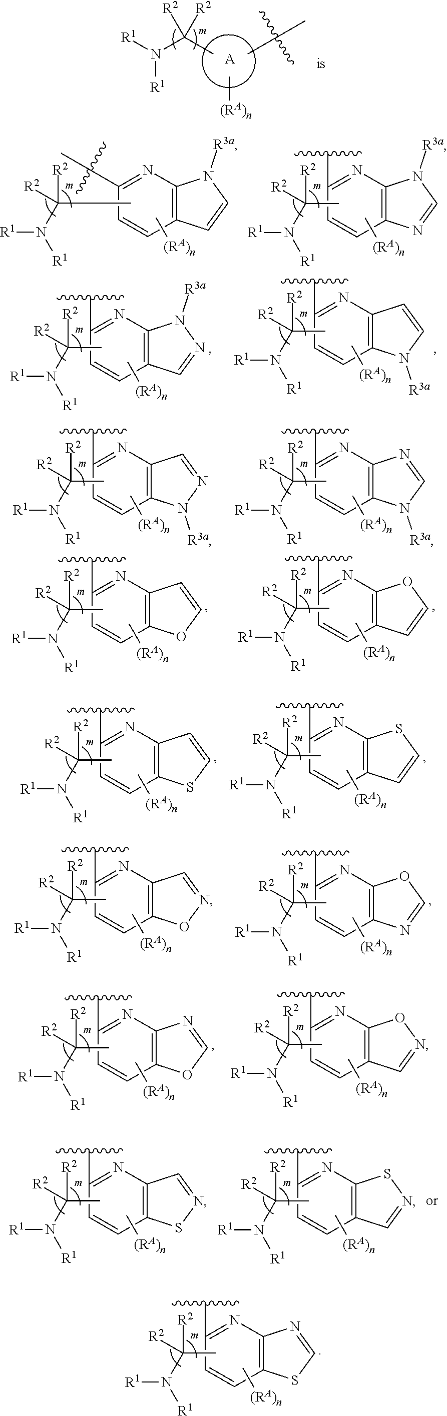

In certain embodiments,

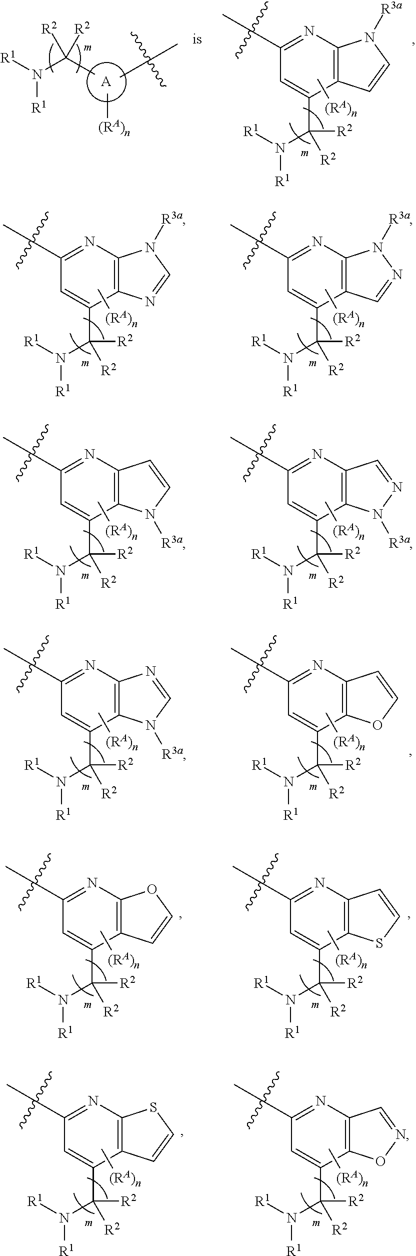

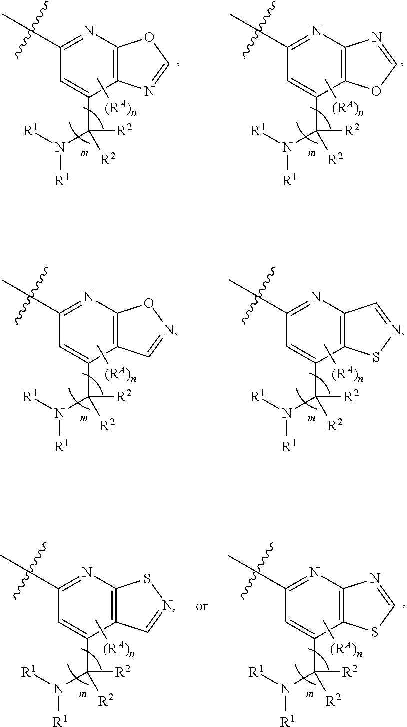

In certain embodiments, R^{3a} is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (CA), pentyl (C₅), or hexyl (C₆)), C₁₋₆ heteroalkyl (e.g., C₁₋₆ alkyl comprising 1-3 heteroatoms selected from O, N, and S), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —(C₁₋₃ alkylene)-(C₃₋₆ carbocyclyl), —(C₁₋₃ alkylene)-(3- to 6-membered heterocyclyl), —(C₁₋₃ alkylene)-(C₆ aryl), —(C₁₋₃ alkylene)-(5- to 6-membered heteroaryl), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkylene, alkenyl, alkynyl, carbocyclyl, heterocyclyl, aryl, or heteroaryl is optionally substituted with one or more R^{u}.

In certain embodiments, R^{3a} is hydrogen, C₁₋₆ alkyl, C₁₋₆ heteroalkyl, C₂₋₆ alkenyl, C₂₋₆ alkynyl, C₃₋₆ carbocyclyl, 3- to 6-membered heterocyclyl, C₆ aryl, 5- to 6-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R^{3a} is hydrogen, C₁₋₆ alkyl, C₁₋₆ heteroalkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(—O)NR^{c}R^{d}, wherein the alkyl, alkenyl, alkynyl, carbocyclyl, heterocyclyl, aryl, or heteroaryl is optionally substituted with one or more R^{u}.

In certain embodiments, R^{3a} is hydrogen, C₁₋₆ alkyl, C₁₋₆ heteroalkyl, C₂₋₆ alkynyl, C₃₋₁₂ carbocyclyl, 3- to 12-membered heterocyclyl, C₆₋₁₀ aryl, 5- to 10-membered heteroaryl, or —(C₁₋₃ alkylene)-(C₆ aryl), wherein the alkyl, alkylne, alkynyl, carbocyclyl, heterocyclyl, aryl, or heteroaryl is optionally substituted with one or more R^{u}.

In certain embodiments, Ring A is 10-membered bicyclic heteroaryl (e.g., bicyclic heteroaryl comprising two 6-membered rings and 1-5 heteroatoms selected from N, O, and S, wherein at least one of the two 6-membered rings is heteoaryl).

In certain embodiments, Ring A is 10-membered bicyclic heteroaryl comprising 1 to 3 nitrogen atoms.

In certain embodiments,

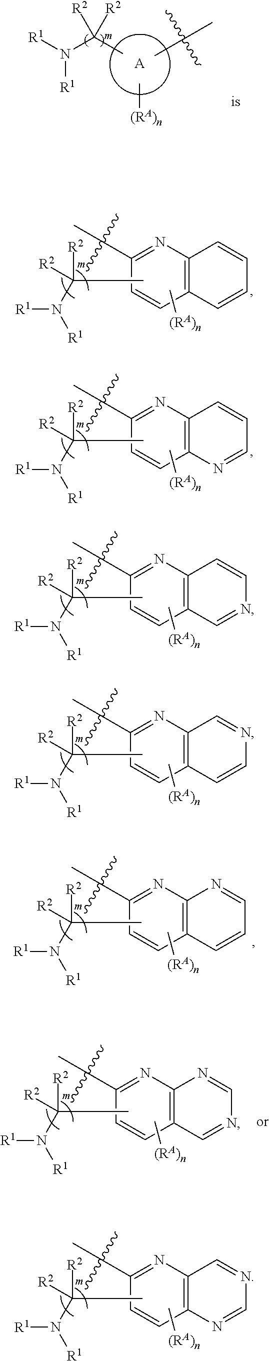

In certain embodiments, Ring A is 9- or 10-membered bicyclic fused ring system comprising one 5- or 6-membered heteoaryl and one 5- to 6-membered heterocyclyl or C₅₋₆ carbocyclyl.

In certain embodiments, Ring A is 9- or 10-membered bicyclic fused ring system comprising one 5- or 6-membered heteoaryl and one C₅₋₆ carbocyclyl.

In certain embodiments,

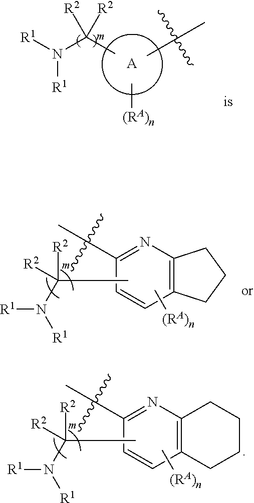

In certain embodiments, each R^{A} is independently oxo, halogen (e.g., —F, —C₁, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), n-propoxy (C₃), i-propoxy (C₃), n-butoxy (C₄), i-butoxy (C₄), S-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, S-butylpentylamino, t-butylpentylamino, n-butylhexylamino, i-butylhexylamino, S-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₁), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(—O) R^{a}, —S(═O)₂R^{a}, —S(═O)₂OR^{b}, —S(═O)₂NR^{c}R^{d}, —NR^{c}S(═O)₂R^{a}, —NR^{c} S(═O) R^{a}, —NR^{c} S(═O)₂OR^{b}, —NR^{c} S(═O)₂NR^{c} R^{a}, —NR^{b}C(—O)NR^{c} R^{a}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(—O)R^{a}, —C(—O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl, is optionally substituted with one or more R^{u}.

In certain embodiments, each R^{A} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl, is optionally substituted with one or more R^{u}.

In certain embodiments, each R^{A} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, C₆ aryl, 5- to 6-membered heteroaryl, or —NR^{c}S(═O)R^{a}, wherein the alkyl, alkoxy, alkylamino, carbocyclyl, heterocyclyl, aryl, or heteroaryl is optionally substituted with one or more R^{u}.

In certain embodiments, each R^{A} is independently oxo, halogen, —NH₂, C₁₋₆ alkyl, C₁₋₆ alkoxy, C₁₋₆ alkylamino, C₂₋₆ alkynyl, C₃₋₆ carbocyclyl, 3- to 6-membered heterocyclyl, C₆ aryl, 5- to 6-membered heteroaryl, or —NR^{c}S(═O) R^{a}, wherein the alkyl, alkoxy, alkylamino, alkynyl, carbocyclyl, heterocyclyl, aryl, or heteroaryl is optionally substituted with one or more R^{u}. In certain embodiments, n is an integer from 0 to 10, as valency permits.

In certain embodiments, n is 0. In certain embodiments, n is 1. In certain embodiments, n is 2. In certain embodiments, n is 3. In certain embodiments, n is 4, as valency permits. In certain embodiments, n is 5, as valency permits. In certain embodiments, n is 6, as valency permits, as valency permits. In certain embodiments, n is 7, as valency permits. In certain embodiments, n is 8. In certain embodiments, n is 9, as valency permits. In certain embodiments, n is 10, as valency permits.

In certain embodiments, two vincinal R^{A}, together with the intervening atoms, form C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), wherein the carbocyclyl or heterocyclyl is optionally substituted with one or more R^{u}.

In certain embodiments, each R^{B} is independently halogen (e.g., —F, —C₁, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), n-propoxy (C₃), i-propoxy (C₃), n-butoxy (C₄), i-butoxy (C₄), S-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, S-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O) R^{a}, —NR^{c} S(═O)₂OR^{b}, —NR^{c}S(═O)₂NR^{c}R^{d}, —NR^{b}C(═O)NR^{c} R⁴, —NR^{b}C(—O)R^{a}, —NR^{b}C(═O)OR^{b}, —OS(═O)₂R^{a}, —OS(═O)₂OR^{b}, —OS(═O)₂NR^{c} R⁴, —OC(═O)R^{a}, —OC(═O)OR^{b}, —OC(═O)NR^{c}R^{d}, —C(═O)R^{a}, —C(═O)OR^{b}, or —C(—O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{B} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{B} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{B} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl, is optionally substituted with one or more R^{u}.

In certain embodiments, each R^{B} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl, is optionally substituted with one or more R^{u}.

In certain embodiments, each R^{B} is independently halogen, C₁₋₆ alkyl, or C₁₋₆ alkoxy.

In certain embodiments, p is an integer from 0 to 3.

In certain embodiments, p is 0. In certain embodiments, p is 1. In certain embodiments, p is 2. In certain embodiments, p is 3.

In certain embodiments, U is —C(R⁴)₂— or —C(═O)—.

In certain embodiments, each R⁴ is independently hydrogen, halogen, —CN, —NO₂, —OH, —NH₂, C₁₋₆ alkyl, C₁₋₆ alkoxy (e.g., methoxy (C₁), ethoxy (C₂), n-propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, S-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered rings and 1-3 heteroatoms selected from N, O, and S), wherein the alkyl, alkoxy, alkylamino, carbocyclyl, or heterocyclyl is optionally substituted with one or more R^{u}.

In certain embodiments, each R⁴ is independently hydrogen or C₁₋₆ alkyl. In certain embodiments, each R⁴ is hydrogen.

In certain embodiments, two R⁴, together with the carbon atom to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered rings and 1-3 heteroatoms selected from N, O, and S), wherein the carbocyclyl or heterocyclyl is optionally substituted with one or more R^{u}.

In certain embodiments, each R^{D} is independently oxo, halogen, —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁;), ethoxy (C₂), n-propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, 1-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{D} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each RDis independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl, is optionally substituted with one or more R^{u}.

In certain embodiments, each R^{D} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl, is optionally substituted with one or more R^{u}.

In certain embodiments, d is an integer selected from 0 to 4.

In certain embodiments, d is 0. In certain embodiments, d is 1. In certain embodiments, d is 2. In certain embodiments, d is 3. In certain embodiments, d is 4.

In certain embodiments, R³ is hydrogen, deuterium, C₁₋₆ haloalkyl (e.g., C₁₋₆ alkyl comprising 1-8 halogen atoms selected from—F, —C₁, and —Br), or C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)).

In certain embodiments, R³ is hydrogen.

In certain embodiments, q is an integer from 0 to 2. In certain embodiments, q is 0. In certain embodiments, q is 1. In certain embodiments, q is 2.

In certain embodiments, the compound is a compound of Formula II-2

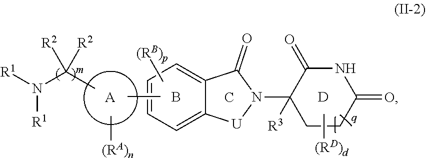

or a pharmaceutically acceptable salt, solvate, or stereoisomer thereof, wherein:
two R¹, together with the nitrogen atom to which they are attached, form 3- to 12-membered heterocyclyl optionally substituted with one or more R^{1b},
each R^{1b} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c} S(═O) R^{a}, —NR^{c} S(═O)₂OR^{b}, —NR^{c}S(═O)₂NR^{c}R^{d}, —NR^{b}C(═O)NR^{c} R^{a}, —NR^{b}C(═O)R^{a}, —NR^{b}C(—O)OR^{b}, —OS(═O)₂R^{a}, —OS(═O)₂OR^{b}, —OS(═O)₂NR^{c}R^{d}, —OC(—O)R^{a}, —OC(═O)OR^{b}, —OC(—O)NR^{c} R⁴, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
two vincinal R^{1b}, together with the intervening atoms, form C₆ aryl or 5- to 6-membered heteroaryl, wherein the aryl or heteroaryl is optionally substituted with one or more R^{u}; each R² is hydrogen;
m is 1;
Ring A is 9- or 10-membered bicyclic fused heteroaryl or 9- or 10-membered bicyclic fused ring system comprising one 5- or 6-membered heteoaryl and one C₅₋₆ carbocyclyl; each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c} S(═O)₂R^{a}, —NR^{c} S(—O) R^{a}, —NR^{c}S(═O)₂OR^{b}, —NR^{c}S(═O)₂NR^{c} R⁴, —NR^{b}C(═O)NR^{c}R^{d}, —NR^{b}C(═O) R^{a}, —NR^{b}C(═O)OR^{b}, —OS(═O)₂R^{a}, —OS(—O)₂OR^{b}, —OS(═O)₂NR^{c} R⁴, —OC(═O)R^{a}, —OC(—O)OR^{b}, —OC(═O)NR^{c} R⁴, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
n is an integer from 0 to 2;
each R^{B} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(—O)R^{a}, —S(═O)₂R^{a}, —S(═O)₂OR^{b}, —S(═O)₂NR^{c}R^{d}, —NR^{c}S(═O)₂R^{a}, —NR^{c} S(═O) R^{a}, —NR^{c}S(═O)₂OR^{b}, —NR^{c}S(═O)₂NR^{c}R^{d}, —NR^{b}C(—O)NR^{c}R^{d}, —NR^{b}C(—O) R^{a}, —NR^{b}C(—O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c} R⁴, —C(—O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
p is an integer from 0 to 3;
U is —CH₂—;
each R^{D} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u};
d is an integer selected from 0 to 4;
R³ is hydrogen, deuterium, C₁₋₆ haloalkyl, or C₁₋₆ alkyl; and
q is 1.

In certain embodiments, the compound is a compound of Formula I:

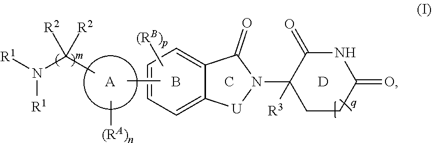

or pharmaceutically acceptable salt, solvate, or stereoisomer thereof, wherein:
each R¹ is independently hydrogen, C₁₋₆ alkyl, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —(C₁₋₆ alkyl)-(C₆₋₁₄ aryl), —(C₁₋₆ alkyl)-(5- to 14-membered heteroaryl), —(C₁₋₆ alkyl)-(C₃₋₁₀ carbocyclyl), —(C₁₋₆ alkyl)-(3- to 10-membered heterocyclyl), wherein the alkyl, alkenyl, alkynyl, carbocyclyl, heterocyclyl, aryl, or heteroaryl is optionally substituted with one or more R^{a},
each R^{1a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c} S(═O)₂R^{a}, —NR^{c} S(═O) R^{a}, —NR^{c} S(═O)₂OR^{b}, —NR^{c}S(═O)₂NR^{c}R^{d}, —NR^{b}C(═O)NR^{c} R⁴, —NR^{b}C(O) R^{a}, —NR^{c} C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(—O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
two R¹, together with the nitrogen atom to which they are attached, form 3- to 12-membered heterocyclyl, wherein the heterocyclyl is optionally substituted with one or more R^{1b},
each R^{1b} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c} R^{a}, —NR^{c} S(—O)₂R^{a}, —NR^{c} S(═O) R^{a}, —NR^{c} S(═O)₂OR^{b}, —NR^{c}S(═O)₂NR^{c} R⁴, —NR^{b}C(═O)NR^{c}R^{d}, —NRDC(═O)R^{a}, —NRDC(—O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c} R⁴, —C(═O)R^{a}, —C(═O)OR^{b}, or —C(—O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
each R² is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(—O)₂OR^{b}, —S(═O)₂NR^{c}R^{d}, —NR^{c} S(═O)₂R^{a}, —NR^{c}S(═O) R^{a}, —NR^{c} S(═O)₂OR^{b}, —NR^{c}S(═O)₂NR^{c}R^{d}, —NR^{b}C(═O)NR^{c} R⁴, —NR^{b}C(—O) R^{a}, —NR^{b}C(═O)OR^{b}, —OS(═O)₂R^{a}, —OS(═O)₂OR^{b}, —OS(═O)₂NR^{c} R⁴, —OC(═O)R^{a}, —OC(═O)OR^{b}, —OC(—O)NR^{c} R⁴, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two R² together form oxo; or
two R², together with the intervening carbon atom(s), form C₃₋₁₀ carbocyclyl or 3- to 10-membered heterocyclyl, wherein the carbocyclyl or heterocyclyl is optionally substituted with one or more R^{u};
m is an integer from 1 to 5;
Ring A is 9- or 10-membered bicyclic heteroaryl;
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(—O)₂R^{a}, —S(═O)₂OR^{b}, —S(═O)₂NR^{c}R^{d}, —NR^{c} S(═O)₂R^{a}, —NR^{c} S(═O) R^{a}, —NR^{c} S(═O)₂OR^{b}, —NR^{c}S(═O)₂NR^{c} R⁴, —NR^{b}C(═O)NR^{c}R^{d}, —NR^{b}C(═O) R^{a}, —NRDC(═O)OR^{b}, —OS(═O)₂R^{a}, —OS(═O)₂OR^{b}, —OS(═O)₂NR^{c}R^{d}, —OC(—O) R^{a}, —OC(═O)OR^{b}, —OC(—O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; n is an integer from 0 to 10, as valency permits;
each R^{B} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c} S(═O) R^{a}, —NR^{c} S(—O)₂OR^{b}, —NR^{c}S(═O)₂NR^{c}R^{d}, —NR^{b}C(═O)NR^{c} R^{a}, —NR^{b}C(═O) R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c} R⁴, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
p is an integer from 0 to 3;
U is —CH₂- or —C(═O)—;
R³ is hydrogen, deuterium, C₁₋₆ haloalkyl, or C₁₋₆ alkyl; and
q is an integer from 0 to 2;
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₀ carbocyclyl, 3- to 10-membered heterocyclyl, —SR^{b}, —S(—O)R^{a}, —S(═O)₂R^{a}, —S(═O)₂OR^{b}, —S(═O)₂NR^{c}R^{d}, —NR^{c}S(═O)₂R^{a}, —NR^{c} S(═O) R^{a}, —NR^{c} S(═O)₂OR^{b}, —NR^{c}S(═O)₂NR^{c}R^{d}, —NR^{b}C(═O)NR^{c} R⁴, —NR^{b}C(═O) R^{a}, —NRDC(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₃₋₁₀ carbocyclyl, and 3- to 6-membered heterocyclyl; or
two R^{u}, together with the one or more intervening atoms, form C₆₋₁₀ aryl, 5- to 10-membered heteroaryl, C₃₋₁₀ carbocyclyl, or 3- to 10-membered heterocyclyl;
each R^{a} is independently C₁₋₆ alkyl, C₂₋₆ alkenyl, C₂₋₆ alkynyl, C₃₋₁₀ carbocyclyl, 3- to 10-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₀ carbocyclyl, 3- to 10-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₀ carbocyclyl, 3- to 10-membered heterocyclyl, C₆₋₁₀ aryl, or 5- to 10-membered heteroaryl; or

R^{c} and R^{d}, together with the nitrogen atom to which they are attached, form 3- to 10-membered heterocyclyl,
wherein each of R^{a}, R^{b}, R^{c}, and R^{d} is independently and optionally substituted with one or more R^{z};
each R^{z} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl,
provided that:
when Ring A is 10-membered bicyclic heteroaryl, Ring A is not isoquinolinyl.

In certain embodiments, each R¹ is independently hydrogen, C₁₋₆ alkyl, C₃₋₆ carbocyclyl, 3- to 6-membered heterocyclyl, —(C₁₋₆ alkyl)-(C₆₋₁₀ aryl), —(C₁₋₆ alkyl)-(5- to 10-membered heteroaryl), —(C₁₋₆ alkyl)-(C₃₋₆ carbocyclyl), or —(C₁₋₆ alkyl)-(3- to 6-membered heterocyclyl), wherein the alkyl, aryl, heteroaryl, carbocyclyl, or heterocyclyl is optionally substituted with one or more R^{1a}.

In certain embodiments each R¹ is independently hydrogen, C₁₋₆ alkyl, —(C₁₋₆ alkyl)-(C₆₋₁₄ aryl), or —(C₁₋₆ alkyl)-(5- to 14-membered heteroaryl), wherein the alkyl, aryl, or heteroaryl is optionally substituted with one or more R^{1a}.

In certain embodiments, each R^{1a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, each R^{1a} is independently halogen, C₁₋₆ alkyl, C₆₋₁₄ aryl, or 5- to 14-membered heteroaryl, wherein the alkyl, aryl, or heteroaryl is optionally substituted with one or more R^{u}.

In certain embodiments, two R¹, together with the nitrogen atom to which they are attached, form 5- or 6-membered heterocyclyl, wherein the heterocyclyl is optionally substituted with one or more R^{1b}.

In certain embodiments, each R^{1b} is independently oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, or —S(═O)₂R^{a}, wherein the alkyl, alkoxy, alkylamino, carbocyclyl, or heterocyclyl is optionally substituted with one or more R^{u}.

In certain embodiments, each R^{1b} is independently halogen, C₁₋₆ alkyl, C₆₋₁₄ aryl, or —S(═O)₂R^{a}, wherein the alkyl or aryl is optionally substituted with one or more R^{u}.

In certain embodiments, each R² is hydrogen.

In certain embodiments, two R² together form oxo.

In certain embodiments, two R², together with the carbon atom to which they are attached, form C₃₋₁₀ carbocyclyl or 3- to 10-membered heterocyclyl.

In certain embodiments, wherein m is 1. In certain embodiments, wherein m is 2. In certain embodiments, wherein m is 3. In certain embodiments, wherein m is 4. In certain embodiments, wherein m is 5.

In certain embodiments, Ring A is 9-membered bicyclic heteroaryl comprising 1 to 4 nitrogen atoms, optionally 0 to 2 sulfur atoms or 0 to 2 oxygen atoms.

In certain embodiments, Ring A is imidazo[1,5-a]pyridinyl, 1H-pyrrolo[2,3-b]pyridinyl, [1,2,4]triazolo[4,3-a]pyridinyl, imidazo[1,2-a]pyridinyl, indolyl, benzo[d]imidazolyl, indazolyl, benzo[d]isoxazolyl, benzo[d]oxazolyl, benzo[d]isothiazolyl, benzo[d]thiazolyl, benzo[b]thiophenyl, or benzofuranyl.

In certain embodiments, Ring A is 9-membered bicyclic heteroaryl comprising 1 to 3 nitrogen atoms.

In certain embodiments,

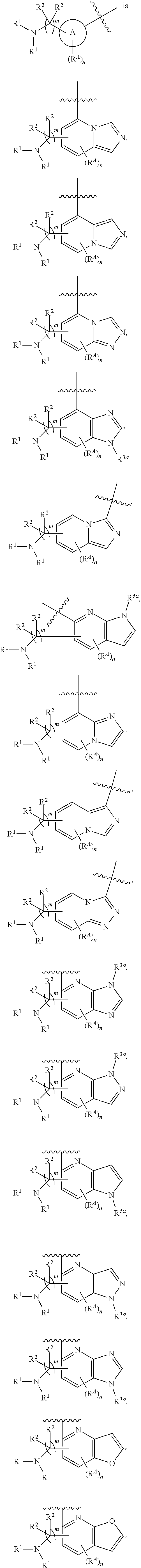

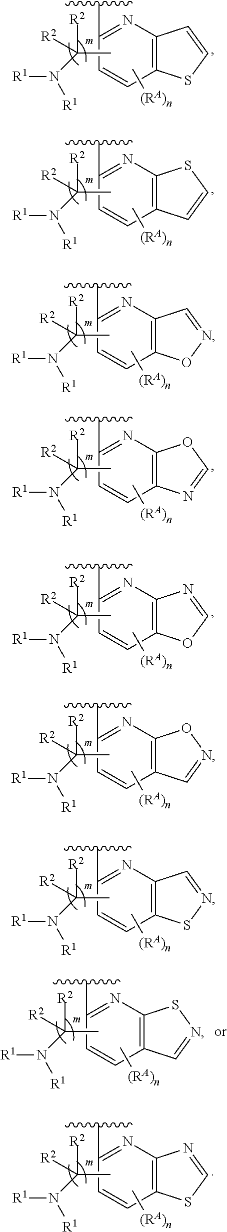

In certain embodiments,

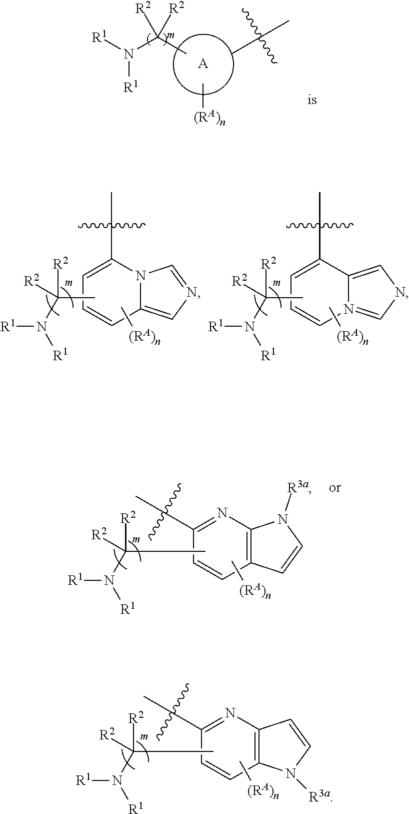

In certain embodiments, Ring A is 10-membered bicyclic heteroaryl comprising 1 to 3 nitrogen atoms, optionally 0 to 2 sulfur atoms or 0 to 2 oxygen atoms.

In certain embodiments, Ring A is 10-membered bicyclic heteroaryl comprising 1 to 3 nitrogen atoms.

In certain embodiments,

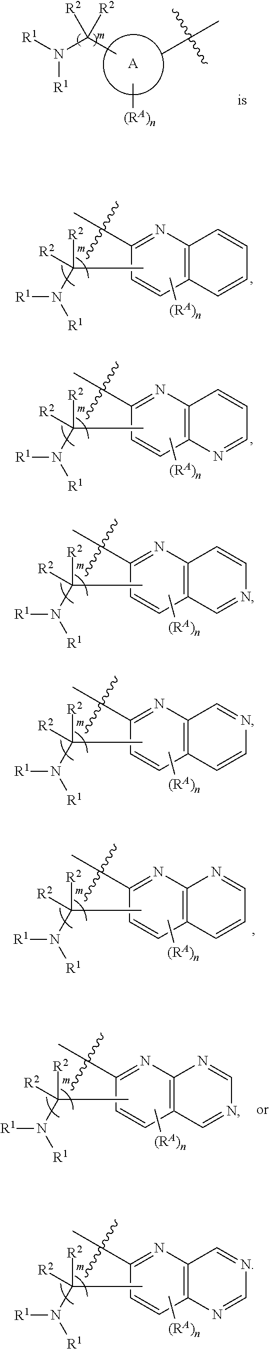

In certain embodiments, Ring A is 6-membered heteroaryl fused with 5- to 6-membered heterocyclyl or C₅₋₆ carbocyclyl.

In certain embodiments,

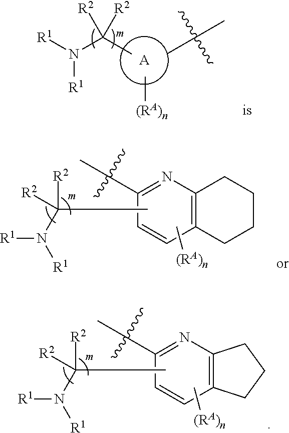

In certain embodiments, the compound is a compound of Formula I-a, I-b, I-c, I-d, I-e, or I-f

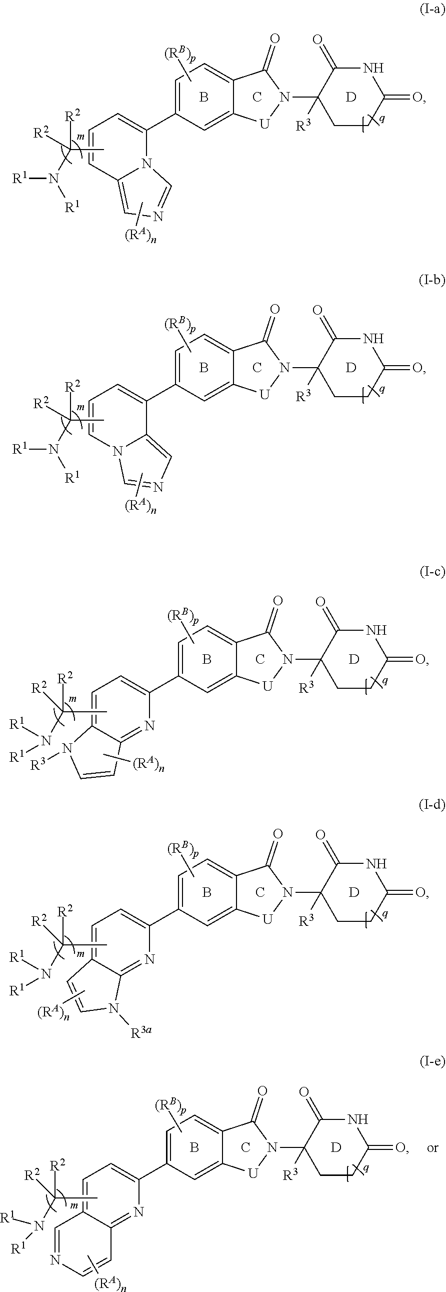

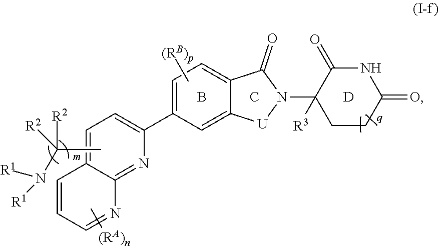

or a pharmaceutically acceptable salt, solvate, or stereoisomer thereof.

In certain embodiments, the compound is a compound of Formula I-a-1, I-b-1, I-c-1, I-d-1, I-e-1, or I-f-1

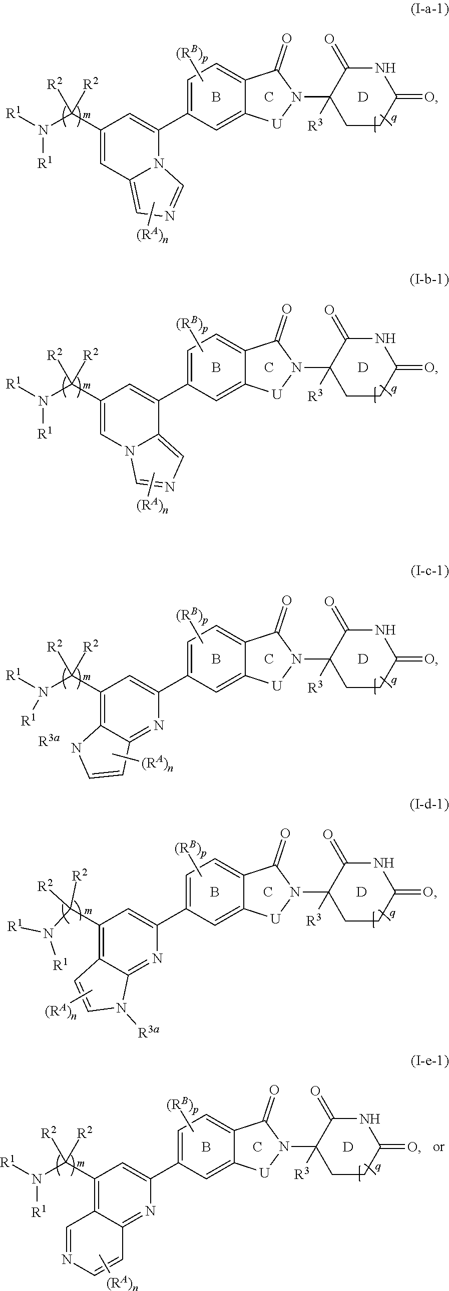

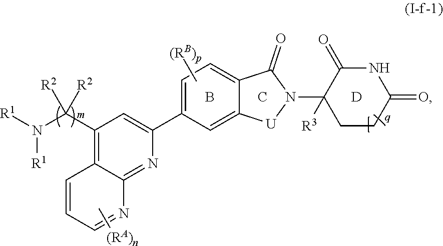

or a pharmaceutically acceptable salt, solvate, or stereoisomer thereof.

In certain embodiments, each R^{A} is independently oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, wherein the alkyl, alkoxy, alkylamino, carbocyclyl, or heterocyclyl is optionally substituted with one or more R^{u}.

In certain embodiments, each R^{A} is independently C₁₋₆ alkyl, wherein the alkyl is optionally substituted with one or more R^{u}.

In certain embodiments, n is an integer from 0 to 8, as valency permits. In certain embodiments, n is an integer from 0 to 6, as valency permits. In certain embodiments, n is an integer from 0 to 5, as valency permits. In certain embodiments, n is an integer from 0 to 4, as valency permits. In certain embodiments, n is an integer from 0 to 3, as valency permits. In certain embodiments, n is an integer from 0 or 1, as valency permits. In certain embodiments, n is 0. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10.

In certain embodiments, each R^{B} is independently halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, wherein the alkyl, alkoxy, alkylamino, carbocyclyl, or heterocyclyl is optionally substituted with one or more R^{u}.

In certain embodiments, each R^{B} is independently halogen or C₁₋₆ alkoxy, wherein the alkoxy is optionally substituted with one or more R^{u}.

In certain embodiments, p is 0 or 1. In certain embodiments, p is 0. In certain embodiments, p is 1.

In certain embodiments, U is —CH₂—. In certain embodiments, U is —C(═O)—.

In certain embodiments, R³ is hydrogen, deuterium, halogen, C₁₋₆ haloalkyl, or C₁₋₆ alkyl. In certain embodiments, R³ is hydrogen, deuterium, or C₁₋₆ alkyl. In certain embodiments, R³ is hydrogen. In certain embodiments, R³ is deuterium. In certain embodiments, R³ is C₁₋₆ alkyl.

In certain embodiments, q is 0. In certain embodiments, q is 1. In certain embodiments, q is 2.

In certain embodiments, each R^{a} is independently C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{a} is independently C₁₋₆ alkyl, C₂₋₆ alkenyl, C₂₋₆ alkynyl, C₃₋₆ carbocyclyl, 3- to 6-membered heterocyclyl, C₆ aryl, or 5- to 6-membered heteroaryl.

In certain embodiments, each R^{a} is independently C₁₋₆ alkyl, C₂₋₆ alkenyl, C₂₋₆ alkynyl, C₃₋₆ carbocyclyl, or 3- to 6-membered heterocyclyl.

In certain embodiments, each R^{a} is independently C₁₋₆ alkyl, C₃₋₆ carbocyclyl, or 3- to 6-membered heterocyclyl, wherein the alkyl, carbocyclyl, or heterocyclyl is optionally substituted with one or more R^{u}.

In certain embodiments, each R^{b} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (CA), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{b} is independently hydrogen, C₁₋₆ alkyl, C₂₋₆ alkenyl, C₂-6 alkynyl, C₃₋₆ carbocyclyl, 3- to 6-membered heterocyclyl, C₆ aryl, or 5- to 6-membered heteroaryl.

In certain embodiments, each R^{b} is independently hydrogen, C₁₋₆ alkyl, C₂₋₆ alkenyl, C₂-6 alkynyl, C₃₋₆ carbocyclyl, or 3- to 6-membered heterocyclyl.

In certain embodiments, each R^{b} is independently hydrogen, C₁₋₆ alkyl, C₃₋₆ carbocyclyl, or 3- to 6-membered heterocyclyl, or C₂₋₆ alkynyl, wherein the alkyl, carbocyclyl, or heterocyclyl is optionally substituted with one or more R^{u}.

In certain embodiments, each R^{c} and each R^{d} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{c} and each R^{d} is independently hydrogen, C₁₋₆ alkyl, C₃₋₆ carbocyclyl, or 3- to 6-membered heterocyclyl, wherein the alkyl, carbocyclyl, or heterocyclyls optionally substituted with one or more R^{u}.

In certain embodiments, R^{c} and R^{d}, together with the nitrogen atom to which they are attached, form 3- to 12-membered heterocyclyl (e.g., heterocyclyl comprising one or two 3- to 8-membered rings and 1-5 heteroatoms selected from N, O, and S), wherein the heterocyclyl is optionally substituted with one or more R^{u}.

In certain embodiments, R^{a}, R^{b}, R^{c}, and R^{d} is independently and optionally substituted with one or more R^{z}.

In certain embodiments, R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl.

In certain embodiments, each R^{u} is independently oxo, halogen, —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c} S(═O)₂OR^{b}, —NR^{c}S(═O)₂NR^{c} R^{a}, —NR^{b}C(═O)NR^{c}R^{d}, —NR^{b}C(═O)R^{a}, —NRDC(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, and 5- to 6-membered heteroaryl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, and 5- to 6-membered heteroaryl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, and 5- to 6-membered heteroaryl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, and 5- to 6-membered heteroaryl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, and 5- to 6-membered heteroaryl.

In certain embodiments, two R^{u}, together with the carbon atom(s) to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), 3- to 6-membered heterocyclyl (e.g., heterocyclyl comprising one 3- to 6-membered ring and 1-3 heteroatoms selected from N, O, and S), C₆ aryl (i.e., phenyl), or 5- to 6-membered heteroaryl (e.g., heteroaryl comprising one 5- or 6-membered ring and 1-3 heteroatoms selected from N, O, and S), wherein the carbocyclyl, heterocyclyl, aryl, or heteroaryl is optionally substituted with one or more R^{z}.

In certain embodiments, two R^{u}, together with the carbon atom(s) to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S), wherein the carbocyclyl or heterocyclyl is optionally substituted with one or more R^{z}.

In certain embodiments, two geminal R^{u}, together with the carbon atom to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S), wherein the carbocyclyl or heterocyclyl is optionally substituted with one or more R^{z}.

Embodiments of the variables in any of the Formulae described herein, as applicable, are described above. Any of the variables can be any moiety as described in the embodiments above. In addition, the combination of any moieties described for any of the variables, as applicable, with any moieties described for any of the remaining variables, are also contemplated.

When a range of values is listed, each discrete value and sub-range within the range are also contemplated. For example, “C₁₋₆ alkyl” is intended to encompass, C₁, C₂, C₃, C₄, C₅, C₆, C₁₋₆, C₁-5, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

In certain embodiments, the compound is selected from the compounds in Table 1 and pharmaceutically acceptable salts thereof:

TABLE 1

| Cmpd | | |
|---|---|---|
| No. | Structure | Compound Name |
| 1 | 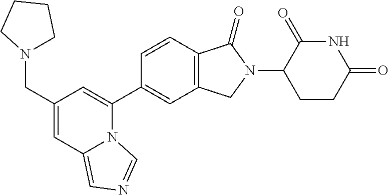 [SMILES: O=C1CCC(N2Cc3cc(-c4cc(CN5CCCC5)cc5cncn45)ccc3C2=O)C(=O)N1] | 3-(1-oxo-5-(7-(pyrrolidin-1- ylmethyl)imidazo[1,5-a]pyridin-5- yl)isoindolin-2-yl)piperidine-2,6- dione |
| 2 | 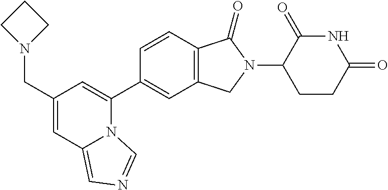 [SMILES: O=C1CCC(N2Cc3cc(-c4cc(CN5CCC5)cc5cncn45)ccc3C2=O)C(=O)N1] | 3-(5-(7-(azetidin-1- ylmethyl)imidazo[1,5-a]pyridin-5- yl)-1-oxoisoindolin-2- yl)piperidine-2,6-dione |
| 3 | 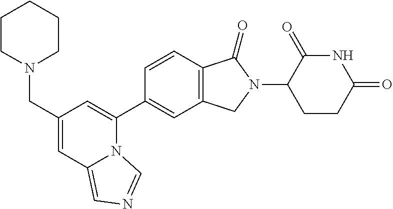 [SMILES: O=C1CCC(N2Cc3cc(-c4cc(CN5CCCCC5)cc5cncn45)ccc3C2=O)C(=O)N1] | 3-(1-oxo-5-(7-(piperidin-1- ylmethyl)imidazo[1,5-a]pyridin-5- yl)isoindolin-2-yl)piperidine-2,6- dione |
| 4 | 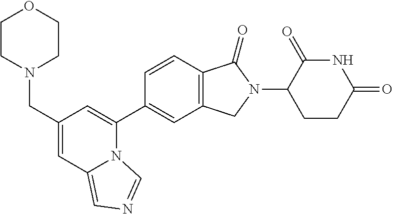 [SMILES: O=C1CCC(N2Cc3cc(-c4cc(CN5CCOCC5)cc5cncn45)ccc3C2=O)C(=O)N1] | 3-(5-(7- (morpholinomethyl)imidazo[1,5- a]pyridin-5-yl)-1-oxoisoindolin-2- yl)piperidine-2,6-dione |
| 5 | 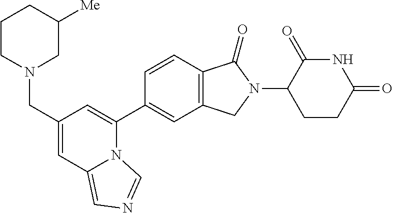 [SMILES: CC1CCCN(Cc2cc(-c3ccc4c(c3)CN(C3CCC(=O)NC3=O)C4=O)n3cncc3c2)C1] | 3-(5-(7-((3-methylpiperidin-1- yl)methyl)imidazo[1,5-a]pyridin-5- yl)-1-oxoisoindolin-2- yl)piperidine-2,6-dione |
| 6 | 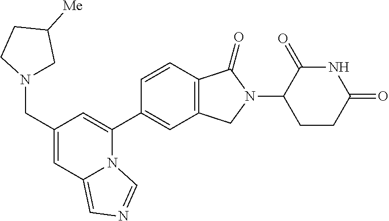 [SMILES: CC1CCN(Cc2cc(-c3ccc4c(c3)CN(C3CCC(=O)NC3=O)C4=O)n3cncc3c2)C1] | 3-(5-(7-((3-methylpyrrolidin-1- yl)methyl)imidazo[1,5-a]pyridin-5- yl)-1-oxoisoindolin-2- yl)piperidine-2,6-dione |
| 7 | 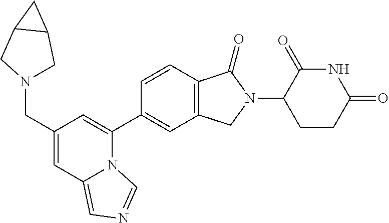 [SMILES: O=C1CCC(N2Cc3cc(-c4cc(CN5CC6CC6C5)cc5cncn45)ccc3C2=O)C(=O)N1] | 3-(5-(7-((3- azabicyclo[3.1.0]hexan-3- yl)methyl)imidazo[1,5-a]pyridin-5- yl)-1-oxoisoindolin-2- yl)piperidine-2,6-dione |
| 8 | 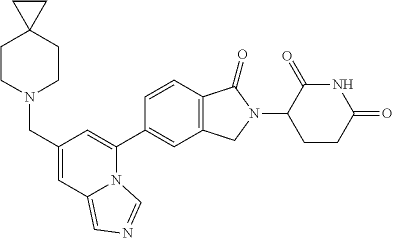 [SMILES: O=C1CCC(N2Cc3cc(-c4cc(CN5CCC6(CC5)CC6)cc5cncn45)ccc3C2=O)C(=O)N1] | 3-(5-(7-((6-azaspiro[2.5]octan-6- yl)methyl)imidazo[1,5-a]pyridin-5- yl)-1-oxoisoindolin-2- yl)piperidine-2,6-dione |
| 9 | 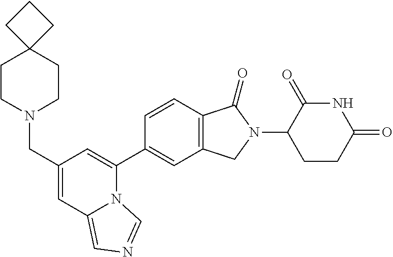 [SMILES: O=C1CCC(N2Cc3cc(-c4cc(CN5CCC6(CCC6)CC5)cc5cncn45)ccc3C2=O)C(=O)N1] | 3-(5-(7-((7-azaspiro[3.5]nonan-7- yl)methyl)imidazo[1,5-a]pyridin-5- yl)-1-oxoisoindolin-2- yl)piperidine-2,6-dione |
| 10 | 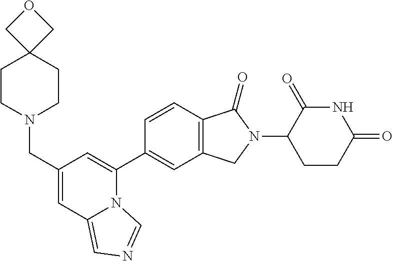 [SMILES: O=C1CCC(N2Cc3cc(-c4cc(CN5CCC6(CC5)COC6)cc5cncn45)ccc3C2=O)C(=O)N1] | 3-(5-(7-((2-oxa-7- azaspiro[3.5]nonan-7- yl)methyl)imidazo[1,5-a]pyridin-5- yl)-1-oxoisoindolin-2- yl)piperidine-2,6-dione |
| 11 | 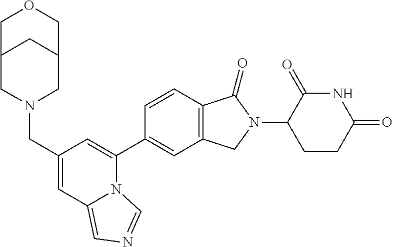 [SMILES: O=C1CCC(N2Cc3cc(-c4cc(CN5CC6COCC(C6)C5)cc5cncn45)ccc3C2=O)C(=O)N1] | 3-(5-(7-((3-oxa-7- azabicyclo[3.3.1]nonan-7- yl)methyl)imidazo[1,5-a]pyridin-5- yl)-1-oxoisoindolin-2- yl)piperidine-2,6-dione |
| 12 | 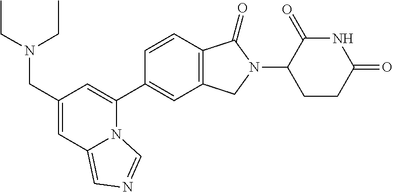 [SMILES: CCN(CC)Cc1cc(-c2ccc3c(c2)CN(C2CCC(=O)NC2=O)C3=O)n2cncc2c1] | 3-(5-(7-((diethylamino)methyl) imidazo[1,5-a]pyridin-5-yl)-1- oxoisoindolin-2-yl)piperidine-2,6- dione |
| 13 | 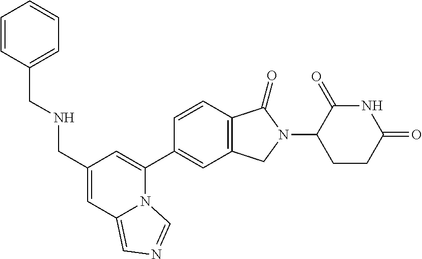 [SMILES: O=C1CCC(N2Cc3cc(-c4cc(CNCc5ccccc5)cc5cncn45)ccc3C2=O)C(=O)N1] | 3-(5-(7- ((benzylamino)methyl)imidazo[1,5- a]pyridin-5-yl)-1-oxoisoindolin-2- yl)piperidine-2,6-dione |
| 14 | 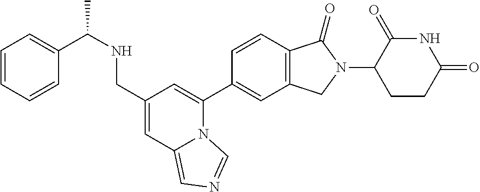 [SMILES: C[C@H](NCc1cc(-c2ccc3c(c2)CN(C2CCC(=O)NC2=O)C3=O)n2cncc2c1)c1ccccc1] | 3-(1-oxo-5-(7-((((S*)-1- phenylethyl)amino)methyl)imidazo [1,5-a]pyridin-5-yl)isoindolin-2- yl)piperidine-2,6-dione |
| 15 | 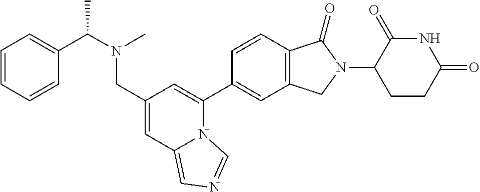 [SMILES: C[C@@H](c1ccccc1)N(C)Cc1cc(-c2ccc3c(c2)CN(C2CCC(=O)NC2=O)C3=O)n2cncc2c1] | 3-(5-(7-((methyl((S*)-1- phenylethyl)amino)methyl)imidazo [1,5-a]pyridin-5-yl)-1- oxoisoindolin-2-yl)piperidine-2,6- dione |
| 16 | 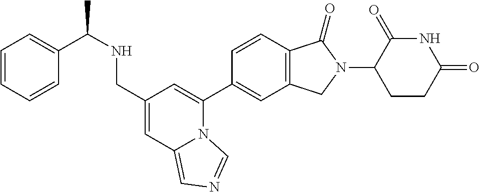 [SMILES: C[C@@H](NCc1cc(-c2ccc3c(c2)CN(C2CCC(=O)NC2=O)C3=O)n2cncc2c1)c1ccccc1] | 3-(1-oxo-5-(7-((((R*)-1- phenylethyl)amino)methyl)imidazo [1,5-a]pyridin-5-yl)isoindolin-2- yl)piperidine-2,6-dione |
| 17 | 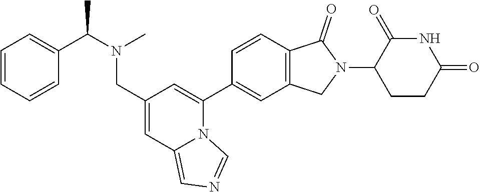 [SMILES: C[C@H](c1ccccc1)N(C)Cc1cc(-c2ccc3c(c2)CN(C2CCC(=O)NC2=O)C3=O)n2cncc2c1] | 3-(5-(7-((methyl((R*)-1- phenylethyl)amino)methyl)imidazo [1,5-a]pyridin-5-yl)-1- oxoisoindolin-2-yl)piperidine-2,6- dione |
| 18 | 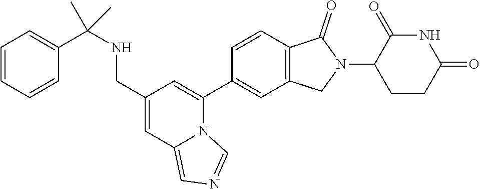 [SMILES: CC(C)(NCc1cc(-c2ccc3c(c2)CN(C2CCC(=O)NC2=O)C3=O)n2cncc2c1)c1ccccc1] | 3-(1-oxo-5-(7-(((2-phenylpropan- 2-yl)amino)methyl)imidazo[1,5- a]pyridin-5-yl)isoindolin-2- yl)piperidine-2,6-dione |
| 19 | 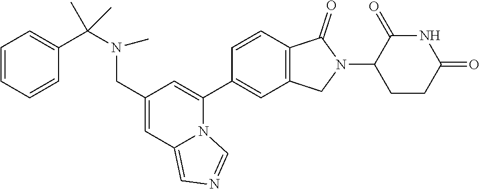 [SMILES: CN(Cc1cc(-c2ccc3c(c2)CN(C2CCC(=O)NC2=O)C3=O)n2cncc2c1)C(C)(C)c1ccccc1] | 3-(5-(7-((methyl(2-phenylpropan- 2-yl)amino)methyl)imidazo[1,5- a]pyridin-5-yl)-1-oxoisoindolin-2- yl)piperidine-2,6-dione |
| 20 | 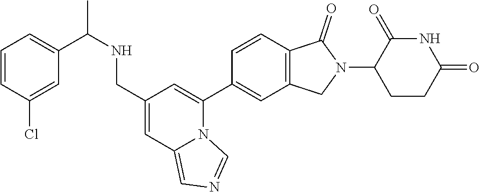 [SMILES: CC(NCc1cc(-c2ccc3c(c2)CN(C2CCC(=O)NC2=O)C3=O)n2cncc2c1)c1cccc(Cl)c1] | 3-(5-(7-(((1-(3-chloro- phenyl)ethyl)amino)methyl)imi- dazo[1,5-a]pyridin-5-yl)-1- oxoisoindolin-2-yl)piperidine-2,6- dione |
| 21 | 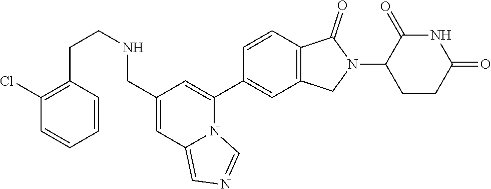 [SMILES: O=C1CCC(N2Cc3cc(-c4cc(CNCCc5ccccc5Cl)cc5cncn45)ccc3C2=O)C(=O)N1] | 3-(5-(7-(((2- chlorophenethyl)amino)methyl)imi- dazo[1,5-a]pyridin-5-yl)-1- oxoisoindolin-2-yl)piperidine-2,6- dione |
| 22 | 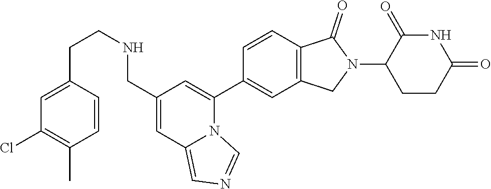 [SMILES: Cc1ccc(CCNCc2cc(-c3ccc4c(c3)CN(C3CCC(=O)NC3=O)C4=O)n3cncc3c2)cc1Cl] | 3-(5-(7-(((3-chloro-4-methyl- phenethyl)amino)methyl)imi- dazo[1,5-a]pyridin-5-yl)-1- oxoisoindolin-2-yl)piperidine-2,6- dione |
| 23 | 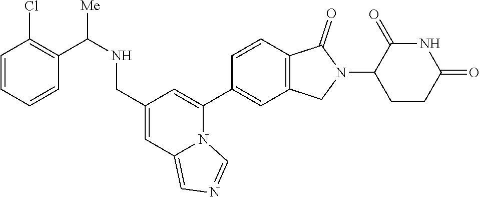 [SMILES: CC(NCc1cc(-c2ccc3c(c2)CN(C2CCC(=O)NC2=O)C3=O)n2cncc2c1)c1ccccc1Cl] | 3-(5-(7-(((1-(2-chloro- phenyl)ethyl)amino)methyl)imi- dazo[1,5-a]pyridin-5-yl)-1- oxoisoindolin-2-yl)piperidine-2,6- dione |
| 24 | 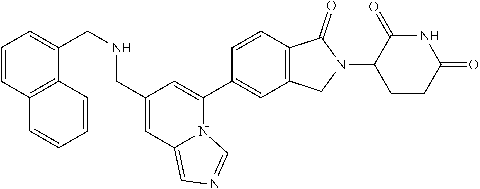 [SMILES: O=C1CCC(N2Cc3cc(-c4cc(CNCc5cccc6ccccc56)cc5cncn45)ccc3C2=O)C(=O)N1] | 3-(5-(7-(((naphthalen-1-ylmeth- yl)amino)methyl)imidazo[1,5- a]pyridin-5-yl)-1-oxoisoindolin- 2-yl)piperidine-2,6-dione |
| 25 | 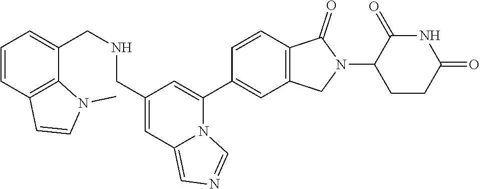 [SMILES: Cn1ccc2cccc(CNCc3cc(-c4ccc5c(c4)CN(C4CCC(=O)NC4=O)C5=O)n4cncc4c3)c21] | 3-(5-(7-((((1-methyl-1H-indol-7- yl)methyl)amino)methyl)imi- dazo[1,5-a]pyridin-5-yl)-1- oxoisoindolin- 2-yl)piperidine-2,6-dione |
| 26 | 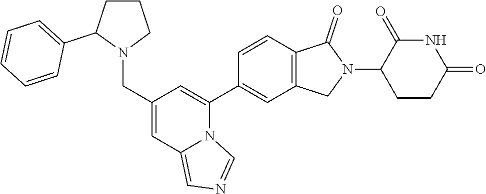 [SMILES: O=C1CCC(N2Cc3cc(-c4cc(CN5CCCC5c5ccccc5)cc5cncn45)ccc3C2=O)C(=O)N1] | 3-(1-oxo-5-(7-((2- phenylpyrrolidin-1- yl)methyl)imidazo[1,5-a]pyridin-5- yl)isoindolin-2-yl)piperidine-2,6- dione |
| 27 | 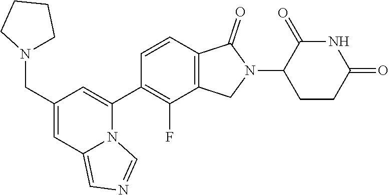 [SMILES: O=C1CCC(N2Cc3c(ccc(-c4cc(CN5CCCC5)cc5cncn45)c3F)C2=O)C(=O)N1] | 3-(4-fluoro-1-oxo-5-(7-(pyrrolidin- 1-ylmethyl)imidazo[1,5-a]pyridin- 5-yl)isoindolin-2-yl)piperidine-2,6- dione |
| 28 | 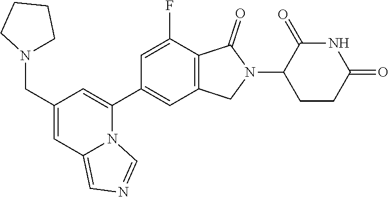 [SMILES: O=C1CCC(N2Cc3cc(-c4cc(CN5CCCC5)cc5cncn45)cc(F)c3C2=O)C(=O)N1] | 3-(7-fluoro-1-oxo-5-(7-(pyrrolidin- 1-ylmethyl)imidazo[1,5-a]pyridin- 5-yl)isoindolin-2-yl)piperidine-2,6- dione |
| 29 | 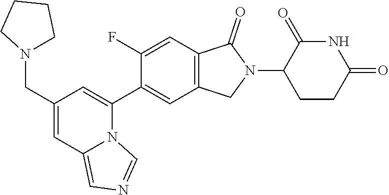 [SMILES: O=C1CCC(N2Cc3cc(-c4cc(CN5CCCC5)cc5cncn45)c(F)cc3C2=O)C(=O)N1] | 3-(6-fluoro-1-oxo-5-(7-(pyrrolidin- 1-ylmethyl)imidazo[1,5-a]pyridin- 5-yl)isoindolin-2-yl)piperidine-2,6- dione |
| 30 | 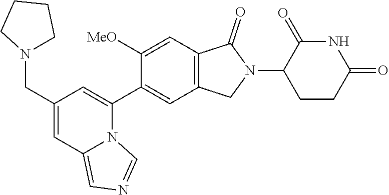 [SMILES: COc1cc2c(cc1-c1cc(CN3CCCC3)cc3cncn13)CN(C1CCC(=O)NC1=O)C2=O] | 3-(6-methoxy-1-oxo-5-(7- (pyrrolidin-1- ylmethyl)imidazo[1,5-a]pyridin-5- yl)isoindolin-2-yl)piperidine-2,6- dione |
| 31 | 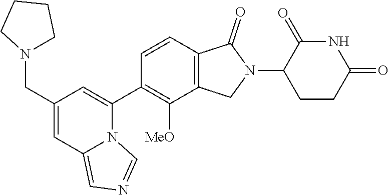 [SMILES: COc1c(-c2cc(CN3CCCC3)cc3cncn23)ccc2c1CN(C1CCC(=O)NC1=O)C2=O] | 3-(4-methoxy-1-oxo-5-(7- (pyrrolidin-1- ylmethyl)imidazo[1,5-a]pyridin-5- yl)isoindolin-2-yl)piperidine-2,6- dione |
| 32 | 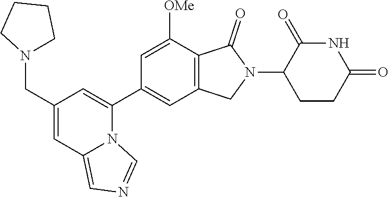 [SMILES: COc1cc(-c2cc(CN3CCCC3)cc3cncn23)cc2c1C(=O)N(C1CCC(=O)NC1=O)C2] | 3-(7-methoxy-1-oxo-5-(7- (pyrrolidin-1- ylmethyl)imidazo[1,5-a]pyridin-5- yl)isoindolin-2-yl)piperidine-2,6- dione |
| 33 | 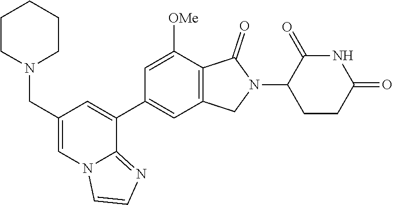 [SMILES: COc1cc(-c2cc(CN3CCCCC3)cn3ccnc23)cc2c1C(=O)N(C1CCC(=O)NC1=O)C2] | 3-(1-oxo-5-(6-(piperidin-1- ylmethyl)imidazo[1,2-a]pyridin-8- yl)isoindolin-2-yl)piperidine-2,6- dione |
| 34 | 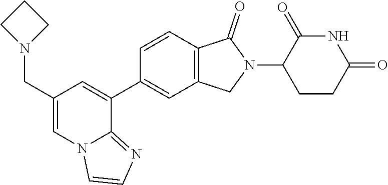 [SMILES: O=C1CCC(N2Cc3cc(-c4cc(CN5CCC5)cn5ccnc45)ccc3C2=O)C(=O)N1] | 3-(5-(6-(azetidin-1- ylmethyl)imidazo[1,2-a]pyridin-8- yl)-1-oxoisoindolin-2- yl)piperidine-2,6-dione |
| 35 | 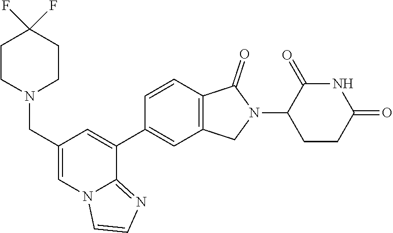 [SMILES: O=C1CCC(N2Cc3cc(-c4cc(CN5CCC(F)(F)CC5)cn5ccnc45)ccc3C2=O)C(=O)N1] | 3-(5-(6-((4,4-difluoropiperidin-1- yl)methyl)imidazo[1,2-a]pyridin-8- yl)-1-oxoisoindolin-2- yl)piperidine-2,6-dione |
| 36 | 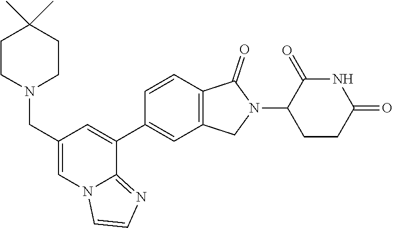 [SMILES: CC1(C)CCN(Cc2cc(-c3ccc4c(c3)CN(C3CCC(=O)NC3=O)C4=O)c3nccn3c2)CC1] | 3-(5-(6-((4,4-dimethylpiperidin-1- yl)methyl)imidazo[1,2-a]pyridin-8- yl)-1-oxoisoindolin-2- yl)piperidine-2,6-dione |
| 37 | 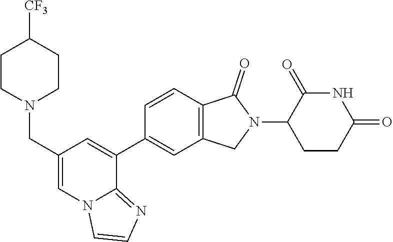 [SMILES: O=C1CCC(N2Cc3cc(-c4cc(CN5CCC(C(F)(F)F)CC5)cn5ccnc45)ccc3C2=O)C(=O)N1] | 3-(1-oxo-5-(6-((4- (trifluoromethyl)piperidin-1- yl)methyl)imidazo[1,2-a]pyridin-8- yl)isoindolin-2-yl)piperidine-2,6- dione |
| 38 | 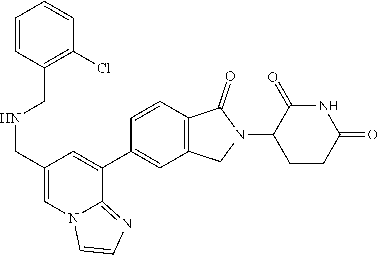 [SMILES: O=C1CCC(N2Cc3cc(-c4cc(CNCc5ccccc5Cl)cn5ccnc45)ccc3C2=O)C(=O)N1] | 3-(5-(6-(((2-chloro- benzyl)amino)methyl)imidazo [1,2-a]pyridin-8-yl)-1- oxoisoindolin-2-yl)piperidine-2,6- dione |
| 39 | 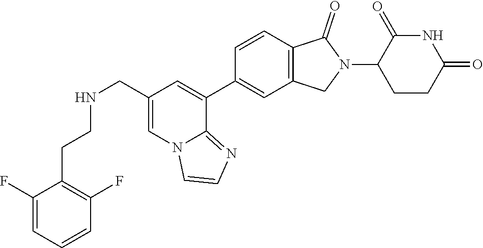 [SMILES: O=C1CCC(N2Cc3cc(-c4cc(CNCCc5c(F)cccc5F)cn5ccnc45)ccc3C2=O)C(=O)N1] | 3-(5-(6-(((2,6-difluoro- phenethyl)amino)methyl)imi- dazo[1,2-a]pyridin-8-yl)-1- oxoisoindolin-2-yl)piperidine-2,6- dione |
| 40 | 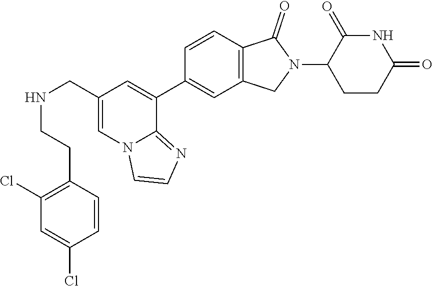 [SMILES: O=C1CCC(N2Cc3cc(-c4cc(CNCCc5ccc(Cl)cc5Cl)cn5ccnc45)ccc3C2=O)C(=O)N1] | 3-(5-(6-(((2,4-dichloro- phenethyl)amino)methyl)imi- dazo[1,2-a]pyridin-8-yl)-1- oxoisoindolin-2-yl)piperidine-2,6- dione |
| 41 | 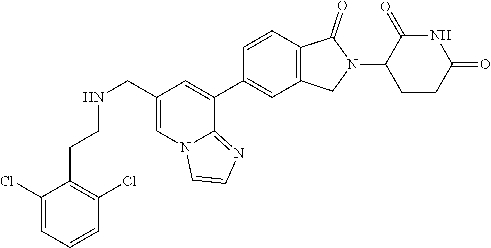 [SMILES: O=C1CCC(N2Cc3cc(-c4cc(CNCCc5c(Cl)cccc5Cl)cn5ccnc45)ccc3C2=O)C(=O)N1] | 3-(5-(6-(((2,6-dichloro- phenethyl)amino)methyl)imi- dazo[1,2-a]pyridin-8-yl)-1- oxoisoindolin-2-yl)piperidine-2,6- dione |
| 42 | 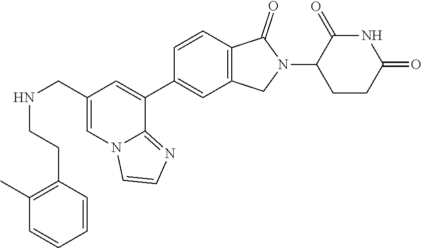 [SMILES: Cc1ccccc1CCNCc1cc(-c2ccc3c(c2)CN(C2CCC(=O)NC2=O)C3=O)c2nccn2c1] | 3-(5-(6-(((2-methyl- phenethyl)amino)methyl)imi- dazo[1,2-a]pyridin-8-yl)-1- oxoisoindolin-2-yl)piperidine-2,6- dione |
| 43 | 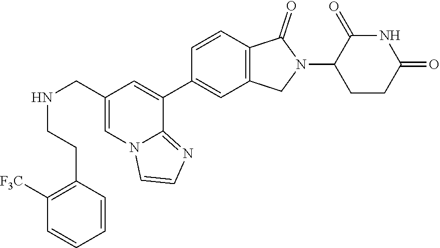 [SMILES: O=C1CCC(N2Cc3cc(-c4cc(CNCCc5ccccc5C(F)(F)F)cn5ccnc45)ccc3C2=O)C(=O)N1] | 3-(1-oxo-5-(6-(((2- (trifluoromethyl)phenethyl)amino) methyl)imidazo[1,2-a]pyridin-8- yl)isoindolin-2-yl)piperidine-2,6- dione |
| 44 | 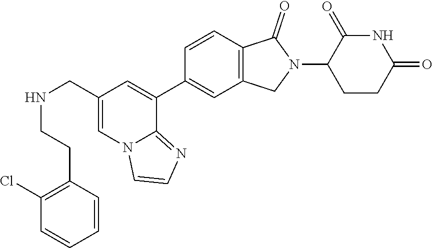 [SMILES: O=C1CCC(N2Cc3cc(-c4cc(CNCCc5ccccc5Cl)cn5ccnc45)ccc3C2=O)C(=O)N1] | 3-(5-(6-(((2-chloro- phenethyl)amino)methyl)imi- dazo[1,2-a]pyridin-8-yl)-1- oxoisoindolin-2-yl)piperidine-2,6- dione |
| 45 | 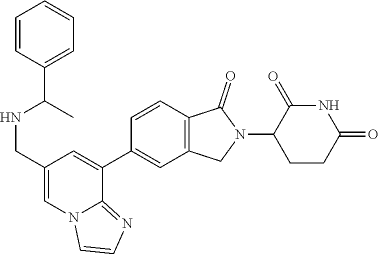 [SMILES: CC(NCc1cc(-c2ccc3c(c2)CN(C2CCC(=O)NC2=O)C3=O)c2nccn2c1)c1ccccc1] | 3-(1-oxo-5-(6-(((1- phenylethyl)amino)methyl)imidazo [1,2-a]pyridin-8-yl)isoindolin-2- yl)piperidine-2,6-dione |
| 46 | 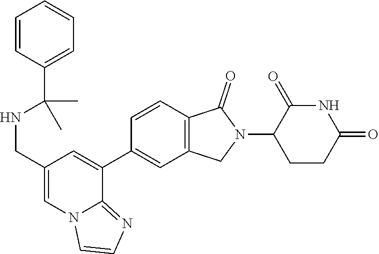 [SMILES: CC(C)(NCc1cc(-c2ccc3c(c2)CN(C2CCC(=O)NC2=O)C3=O)c2nccn2c1)c1ccccc1] | 3-(1-oxo-5-(6-(((2-phenylpropan- 2-yl)amino)methyl)imidazo[1,2- a]pyridin-8-yl)isoindolin-2- yl)piperidine-2,6-dione |
| 47 | 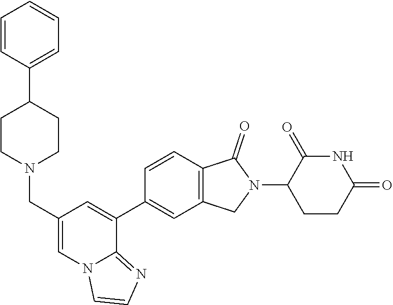 [SMILES: O=C1CCC(N2Cc3cc(-c4cc(CN5CCC(c6ccccc6)CC5)cn5ccnc45)ccc3C2=O)C(=O)N1] | 3-(1-oxo-5-(6-((4-phenylpiperidin- 1-yl)methyl)imidazo[1,2-a]pyridin- 8-yl)isoindolin-2-yl)piperidine-2,6- dione |
| 48 | 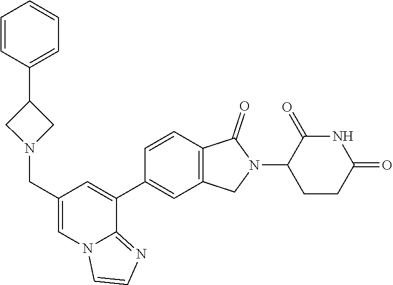 [SMILES: O=C1CCC(N2Cc3cc(-c4cc(CN5CC(c6ccccc6)C5)cn5ccnc45)ccc3C2=O)C(=O)N1] | 3-(1-oxo-5-(6-((3-phenylazetidin- 1-yl)methyl)imidazo[1,2-a]pyridin- 8-yl)isoindolin-2-yl)piperidine-2,6- dione |
| 49 | 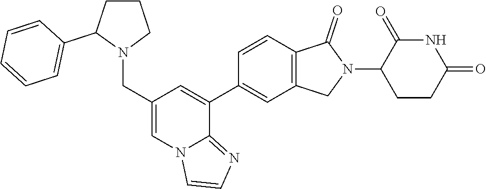 [SMILES: O=C1CCC(N2Cc3cc(-c4cc(CN5CCCC5c5ccccc5)cn5ccnc45)ccc3C2=O)C(=O)N1] | 3-(1-oxo-5-(6-((2- phenylpyrrolidin-1- yl)methyl)imidazo[1,2-a]pyridin-8- yl)isoindolin-2-yl)piperidine-2,6- dione |
| 50 | 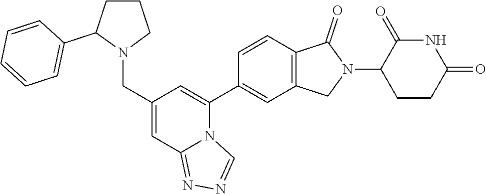 [SMILES: O=C1CCC(N2Cc3cc(-c4cc(CN5CCCC5c5ccccc5)cc5nncn45)ccc3C2=O)C(=O)N1] | 3-(1-oxo-5-(7-((2- phenylpyrrolidin-1-yl)methyl)- [1,2,4]triazolo[4,3-a]pyridin-5- yl)isoindolin-2-yl)piperidine-2,6- dione |
| 51 | 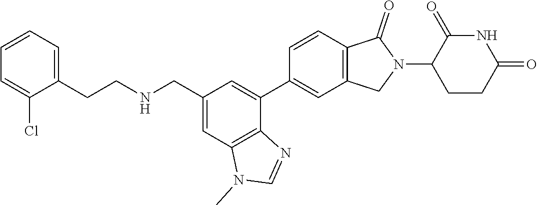 [SMILES: Cn1cnc2c(-c3ccc4c(c3)CN(C3CCC(=O)NC3=O)C4=O)cc(CNCCc3ccccc3Cl)cc21] | 3-(5-(6-(((2- chlorophenethyl)amino)methyl)-1- methyl-1H-benzo[d]imidazol-4- yl)-1-oxoisoindolin-2- yl)piperidine-2,6-dione |
| 52 | 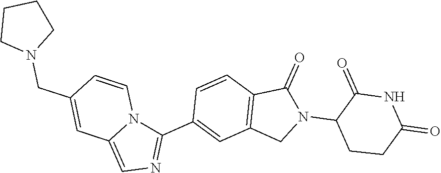 [SMILES: O=C1CCC(N2Cc3cc(-c4ncc5cc(CN6CCCC6)ccn45)ccc3C2=O)C(=O)N1] | 3-(1-oxo-5-(7-(pyrrolidin-1- ylmethyl)imidazo[1,5-a]pyridin-3- yl)isoindolin-2-yl)piperidine-2,6- dione |
| 53 | 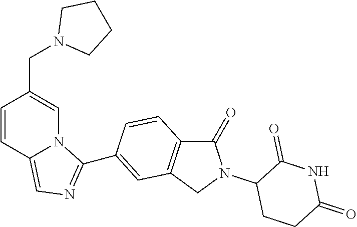 [SMILES: O=C1CCC(N2Cc3cc(-c4ncc5ccc(CN6CCCC6)cn45)ccc3C2=O)C(=O)N1] | 3-(1-oxo-5-(6-(pyrrolidin-1- ylmethyl)imidazo[1,5-a]pyridin-3- yl)isoindolin-2-yl)piperidine-2,6- dione |
| 54 | 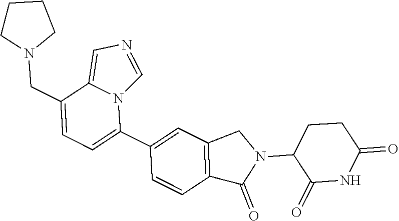 [SMILES: O=C1CCC(N2Cc3cc(-c4ccc(CN5CCCC5)c5cncn45)ccc3C2=O)C(=O)N1] | 3-(1-oxo-5-(8-(pyrrolidin-1- ylmethyl)imidazo[1,5-a]pyridin-5- yl)isoindolin-2-yl)piperidine-2,6- dione |
| 55 | 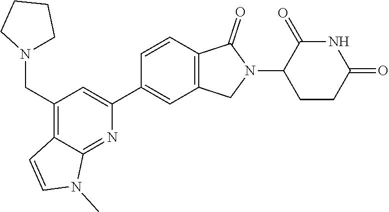 [SMILES: Cn1ccc2c(CN3CCCC3)cc(-c3ccc4c(c3)CN(C3CCC(=O)NC3=O)C4=O)nc21] | 3-(5-(1-methyl-4-(pyrrolidin-1- ylmethyl)-1H-pyrrolo[2,3- b]pyridin-6-yl)-1-oxoisoindolin-2- yl)piperidine-2,6-dione |
| 56 | 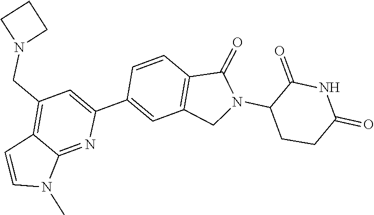 [SMILES: Cn1ccc2c(CN3CCC3)cc(-c3ccc4c(c3)CN(C3CCC(=O)NC3=O)C4=O)nc21] | 3-(5-(4-(azetidin-1-ylmethyl)-1- methyl-1H-pyrrolo[2,3-b]pyridin- 6-yl)-1-oxoisoindolin-2- yl)piperidine-2,6-dione |
| 57 | 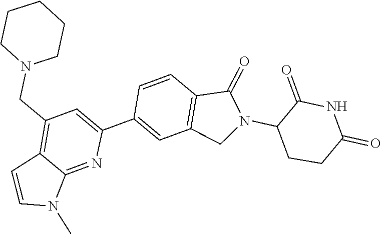 [SMILES: Cn1ccc2c(CN3CCCCC3)cc(-c3ccc4c(c3)CN(C3CCC(=O)NC3=O)C4=O)nc21] | 3-(5-(1-methyl-4-(piperidin-1- ylmethyl)-1H-pyrrolo[2,3- b]pyridin-6-yl)-1-oxoisoindolin-2- yl)piperidine-2,6-dione |
| 58 | 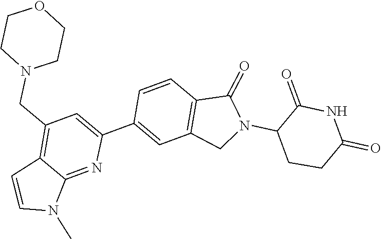 [SMILES: Cn1ccc2c(CN3CCOCC3)cc(-c3ccc4c(c3)CN(C3CCC(=O)NC3=O)C4=O)nc21] | 3-(5-(1-methyl-4- (morpholinomethyl)-1H- pyrrolo[2,3-b]pyridin-6-yl)-1- oxoisoindolin-2-yl)piperidine-2,6- dione |
| 59 | 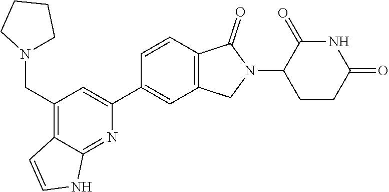 [SMILES: O=C1CCC(N2Cc3cc(-c4cc(CN5CCCC5)c5cc[nH]c5n4)ccc3C2=O)C(=O)N1] | 3-(1-oxo-5-(4-(pyrrolidin-1- ylmethyl)-1H-pyrrolo[2,3- b]pyridin-6-yl)isoindolin-2- yl)piperidine-2,6-dione |
| 60 | 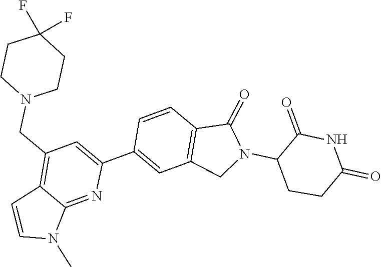 [SMILES: Cn1ccc2c(CN3CCC(F)(F)CC3)cc(-c3ccc4c(c3)CN(C3CCC(=O)NC3=O)C4=O)nc21] | 3-(5-(4-((4,4-difluoropiperidin-1- yl)methyl)-1-methyl-1H- pyrrolo[2,3-b]pyridin-6-yl)-1- oxoisoindolin-2-yl)piperidine-2,6- dione |
| 61 | 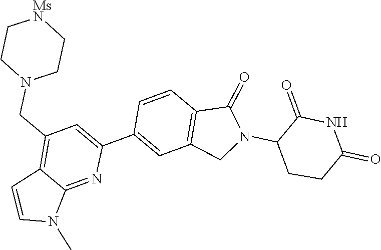 | 3-(5-(1-methyl-4-((4- (methylsulfonyl)piperazin-1- yl)methyl)-1H-pyrrolo[2,3- b]pyridin-6-yl)-1-oxoisoindolin-2- yl)piperidine-2,6-dione |
| 62 | 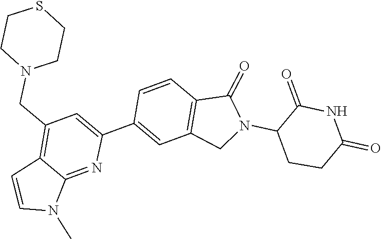 [SMILES: Cn1ccc2c(CN3CCSCC3)cc(-c3ccc4c(c3)CN(C3CCC(=O)NC3=O)C4=O)nc21] | 3-(5-(1-methyl-4- (thiomorpholinomethyl)-1H- pyrrolo[2,3-b]pyridin-6-yl)-1- oxoisoindolin-2-yl)piperidine-2,6- dione |
| 63 | 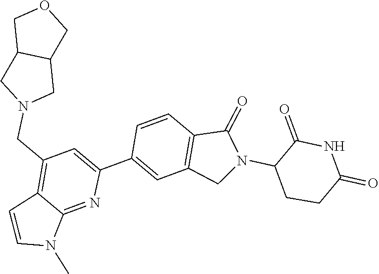 [SMILES: Cn1ccc2c(CN3CC4COCC4C3)cc(-c3ccc4c(c3)CN(C3CCC(=O)NC3=O)C4=O)nc21] | 3-(5-(1-methyl-4-((tetrahydro-1H- furo[3,4-c]pyrrol-5(3H)- yl)methyl)-1H-pyrrolo[2,3- b]pyridin-6-yl)-1-oxoisoindolin-2- yl)piperidine-2,6-dione |
| 64 | 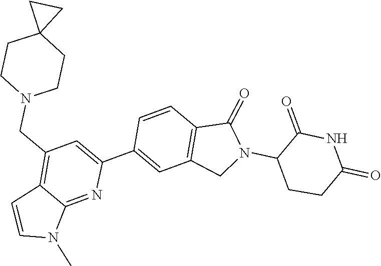 [SMILES: Cn1ccc2c(CN3CCC4(CC3)CC4)cc(-c3ccc4c(c3)CN(C3CCC(=O)NC3=O)C4=O)nc21] | 3-(5-(4-((6-azaspiro[2.5]octan-6- yl)methyl)-1-methyl-1H- pyrrolo[2,3-b]pyridin-6-yl)-1- oxoisoindolin-2-yl)piperidine-2,6- dione |
| 65 | 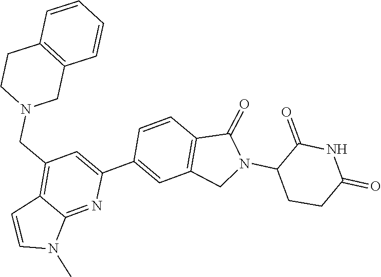 [SMILES: Cn1ccc2c(CN3CCc4ccccc4C3)cc(-c3ccc4c(c3)CN(C3CCC(=O)NC3=O)C4=O)nc21] | 3-(5-(4-((3,4-dihydroisoquinolin- 2(1H)-yl)methyl)-1-methyl-1H- pyrrolo[2,3-b]pyridin-6-yl)-1- oxoisoindolin-2-yl)piperidine-2,6- dione |
| 66 | 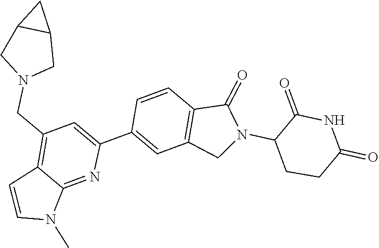 [SMILES: Cn1ccc2c(CN3CC4CC4C3)cc(-c3ccc4c(c3)CN(C3CCC(=O)NC3=O)C4=O)nc21] | 3-(5-(4-((3- azabicyclo[3.1.0]hexan-3- yl)methyl)-1-methyl-1H- pyrrolo[2,3-b]pyridin-6-yl)-1- oxoisoindolin-2-yl)piperidine-2,6- dione |
| 67 | 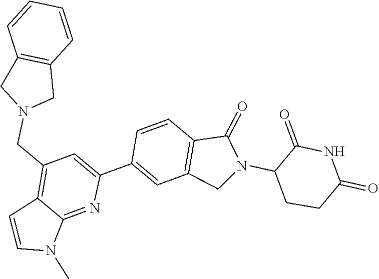 [SMILES: Cn1ccc2c(CN3Cc4ccccc4C3)cc(-c3ccc4c(c3)CN(C3CCC(=O)NC3=O)C4=O)nc21] | 3-(5-(4-(isoindolin-2-ylmethyl)-1- methyl-1H-pyrrolo[2,3-b]pyridin- 6-yl)-1-oxoisoindolin-2- yl)piperidine-2,6-dione |
| 68 | 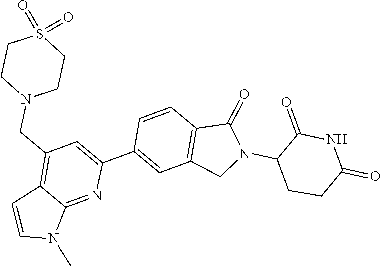 [SMILES: Cn1ccc2c(CN3CCS(=O)(=O)CC3)cc(-c3ccc4c(c3)CN(C3CCC(=O)NC3=O)C4=O)nc21] | 3-(5-(4-((1,1- dioxidothiomorpholino)methyl)-1- methyl-1H-pyrrolo[2,3-b]pyridin- 6-yl)-1-oxoisoindolin-2- yl)piperidine-2,6-dione |
| 69 | 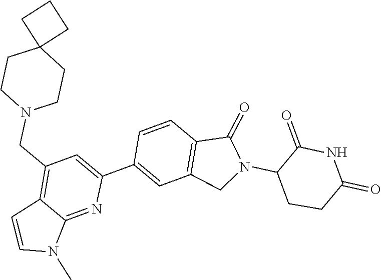 [SMILES: Cn1ccc2c(CN3CCC4(CCC4)CC3)cc(-c3ccc4c(c3)CN(C3CCC(=O)NC3=O)C4=O)nc21] | 3-(5-(4-((7-azaspiro[3.5]nonan-7- yl)methyl)-1-methyl-1H- pyrrolo[2,3-b]pyridin-6-yl)-1- oxoisoindolin-2-yl)piperidine-2,6- dione |
| 70 | 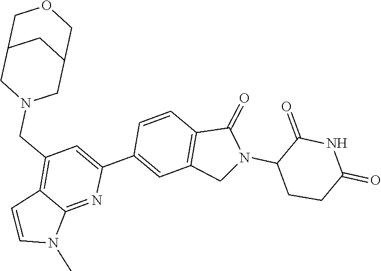 [SMILES: Cn1ccc2c(CN3CC4COCC(C4)C3)cc(-c3ccc4c(c3)CN(C3CCC(=O)NC3=O)C4=O)nc21] | 3-(5-(4-((3-oxa-7- azabicyclo[3.3.1]nonan-7- yl)methyl)-1-methyl-1H- pyrrolo[2,3-b]pyridin-6-yl)-1- oxoisoindolin-2-yl)piperidine-2,6- dione |
| 71 | 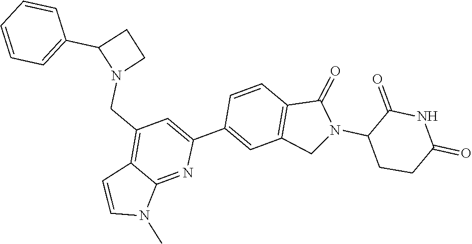 [SMILES: Cn1ccc2c(CN3CCC3c3ccccc3)cc(-c3ccc4c(c3)CN(C3CCC(=O)NC3=O)C4=O)nc21] | 3-(5-(1-methyl-4-((2- phenylazetidin-1-yl)methyl)-1H- pyrrolo[2,3-b]pyridin-6-yl)-1- oxoisoindolin-2-yl)piperidine-2,6- dione |
| 72 | 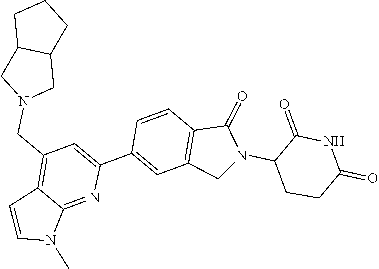 [SMILES: Cn1ccc2c(CN3CC4CCCC4C3)cc(-c3ccc4c(c3)CN(C3CCC(=O)NC3=O)C4=O)nc21] | 3-(5-(4- ((hexahydrocyclopenta[c]pyrrol- 2(1H)-yl)methyl)-1-methyl-1H- pyrrolo[2,3-b]pyridin-6-yl)-1- oxoisoindolin-2-yl)piperidine-2,6- dione |
| 73 | 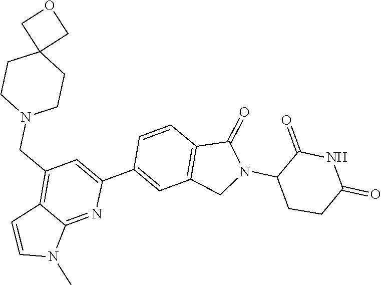 [SMILES: Cn1ccc2c(CN3CCC4(CC3)COC4)cc(-c3ccc4c(c3)CN(C3CCC(=O)NC3=O)C4=O)nc21] | 3-(5-(4-((2-oxa-7- azaspiro[3.5]nonan-7-yl)methyl)-1- methyl-1H-pyrrolo[2,3-b]pyridin- 6-yl)-1-oxoisoindolin-2- yl)piperidine-2,6-dione |
| 74 | 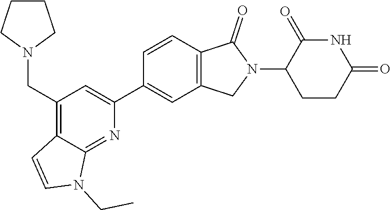 [SMILES: CCn1ccc2c(CN3CCCC3)cc(-c3ccc4c(c3)CN(C3CCC(=O)NC3=O)C4=O)nc21] | 3-(5-(1-ethyl-4-(pyrrolidin-1- ylmethyl)-1H-pyrrolo[2,3- b]pyridin-6-yl)-1-oxoisoindolin-2- yl)piperidine-2,6-dione |
| 75 | 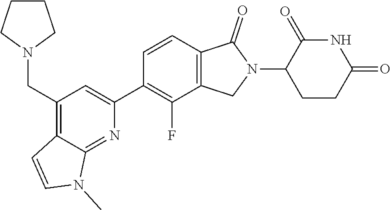 [SMILES: Cn1ccc2c(CN3CCCC3)cc(-c3ccc4c(c3F)CN(C3CCC(=O)NC3=O)C4=O)nc21] | 3-(4-fluoro-5-(1-methyl-4- (pyrrolidin-1-ylmethyl)-1H- pyrrolo[2,3-b]pyridin-6-yl)-1- oxoisoindolin-2-yl)piperidine-2,6- dione |
| 76 | 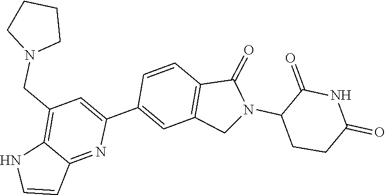 [SMILES: O=C1CCC(N2Cc3cc(-c4cc(CN5CCCC5)c5[nH]ccc5n4)ccc3C2=O)C(=O)N1] | 3-(1-oxo-5-(7-(pyrrolidin-1- ylmethyl)-1H-pyrrolo[3,2- b]pyridin-5-yl)isoindolin-2- yl)piperidine-2,6-dione |
| 77 | 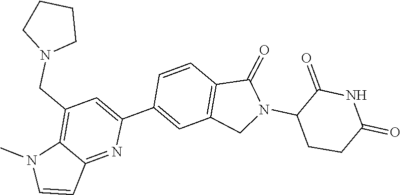 [SMILES: Cn1ccc2nc(-c3ccc4c(c3)CN(C3CCC(=O)NC3=O)C4=O)cc(CN3CCCC3)c21] | 3-(5-(1-methyl-7-(pyrrolidin-1- ylmethyl)-1H-pyrrolo[3,2- b]pyridin-5-yl)-1-oxoisoindolin-2- yl)piperidine-2,6-dione |
| 78 | 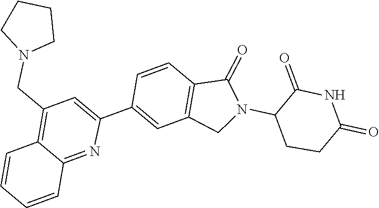 [SMILES: O=C1CCC(N2Cc3cc(-c4cc(CN5CCCC5)c5ccccc5n4)ccc3C2=O)C(=O)N1] | 3-(1-oxo-5-(4-(pyrrolidin-1- ylmethyl)quinolin-2-yl)isoindolin- 2-yl)piperidine-2,6-dione |
| 79 | 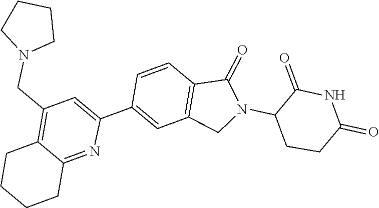 [SMILES: O=C1CCC(N2Cc3cc(-c4cc(CN5CCCC5)c5c(n4)CCCC5)ccc3C2=O)C(=O)N1] | 3-(1-oxo-5-(4-(pyrrolidin-1- ylmethyl)-5,6,7,8- tetrahydroquinolin-2-yl)isoindolin- 2-yl)piperidine-2,6-dione |
| 80 | 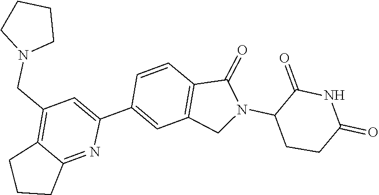 [SMILES: O=C1CCC(N2Cc3cc(-c4cc(CN5CCCC5)c5c(n4)CCC5)ccc3C2=O)C(=O)N1] | 3-(1-oxo-5-(4-(pyrrolidin-1- ylmethyl)-6,7-dihydro-5H- cyclopenta[b]pyridin-2- yl)isoindolin-2-yl)piperidine-2,6- dione |
| 81 | 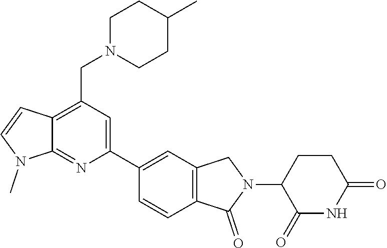 [SMILES: CC1CCN(Cc2cc(-c3ccc4c(c3)CN(C3CCC(=O)NC3=O)C4=O)nc3c2ccn3C)CC1] | 3-(5-(1-methyl-4-((4- methylpiperidin-1-yl)methyl)-1H- pyrrolo[2,3-b]pyridin-6-yl)-1- oxoisoindolin-2-yl)piperidine-2,6- dione |
| 82 | 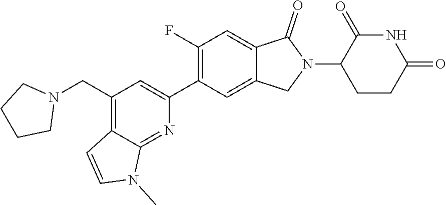 [SMILES: Cn1ccc2c(CN3CCCC3)cc(-c3cc4c(cc3F)C(=O)N(C3CCC(=O)NC3=O)C4)nc21] | 3-(6-fluoro-5-(1-methyl-4- (pyrrolidin-1-ylmethyl)-1H- pyrrolo[2,3-b]pyridin-6-yl)-1- oxoisoindolin-2-yl)piperidine-2,6- dione |
| 83 | 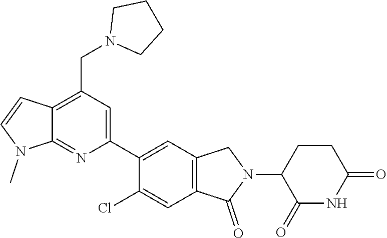 [SMILES: Cn1ccc2c(CN3CCCC3)cc(-c3cc4c(cc3Cl)C(=O)N(C3CCC(=O)NC3=O)C4)nc21] | 3-(6-chloro-5-(1-methyl-4- (pyrrolidin-1-ylmethyl)-1H- pyrrolo[2,3-b]pyridin-6-yl)-1- oxoisoindolin-2-yl)piperidine-2,6- dione |
| 84 | 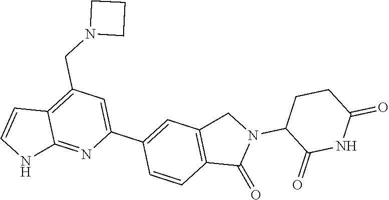 [SMILES: O=C1CCC(N2Cc3cc(-c4cc(CN5CCC5)c5cc[nH]c5n4)ccc3C2=O)C(=O)N1] | 3-(5-(4-(azetidin-1-ylmethyl)-1H- pyrrolo[2,3-b]pyridin-6-yl)-1- oxoisoindolin-2-yl)piperidine-2,6- dione |
| 85 | 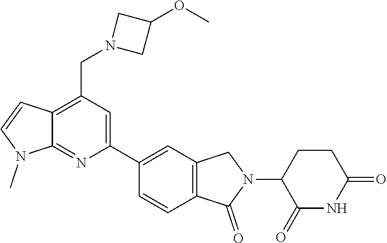 [SMILES: COC1CN(Cc2cc(-c3ccc4c(c3)CN(C3CCC(=O)NC3=O)C4=O)nc3c2ccn3C)C1] | 3-(5-(4-((3-methoxyazetidin-1- yl)methyl)-1-methyl-1H- pyrrolo[2,3-b]pyridin-6-yl)-1- oxoisoindolin-2-yl)piperidine-2,6- dione |
| 86 | 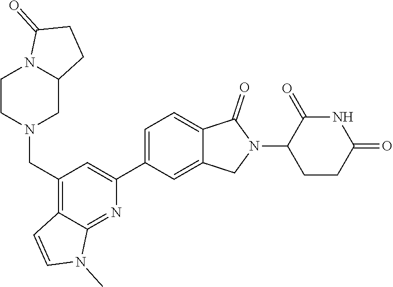 [SMILES: Cn1ccc2c(CN3CCN4C(=O)CCC4C3)cc(-c3ccc4c(c3)CN(C3CCC(=O)NC3=O)C4=O)nc21] | 3-(5-(1-methyl-4-((6- oxohexahydropyrrolo[1,2- a]pyrazin-2(1H)-yl)methyl)-1H- pyrrolo[2,3-b]pyridin-6-yl)-1- oxoisoindolin-2-yl)piperidine-2,6- dione |
| 87 | 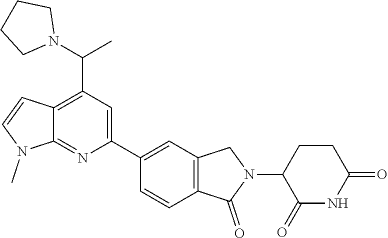 [SMILES: CC(c1cc(-c2ccc3c(c2)CN(C2CCC(=O)NC2=O)C3=O)nc2c1ccn2C)N1CCCC1] | 3-(5-(1-methyl-4-(1-(pyrrolidin-1- yl)ethyl)-1H-pyrrolo[2,3-b]pyridin- 6-yl)-1-oxoisoindolin-2- yl)piperidine-2,6-dione |
| 88 | 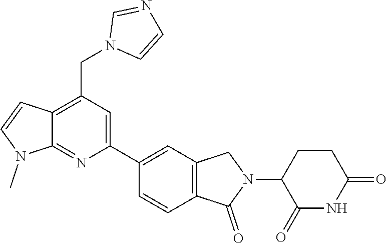 [SMILES: Cn1ccc2c(Cn3ccnc3)cc(-c3ccc4c(c3)CN(C3CCC(=O)NC3=O)C4=O)nc21] | 3-(5-(4-((1H-imidazol-1- yl)methyl)-1-methyl-1H- pyrrolo[2,3-b]pyridin-6-yl)-1- oxoisoindolin-2-yl)piperidine-2,6- dione |
| 89 | 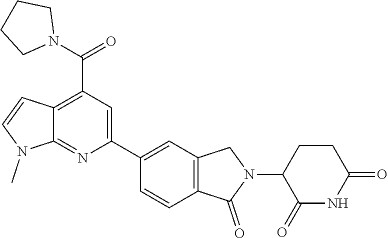 [SMILES: Cn1ccc2c(C(=O)N3CCCC3)cc(-c3ccc4c(c3)CN(C3CCC(=O)NC3=O)C4=O)nc21] | 3-(5-(1-methyl-4-(pyrrolidine-1- carbonyl)-1H-pyrrolo[2,3- b]pyridin-6-yl)-1-oxoisoindolin-2- yl)piperidine-2,6-dione |
| 90 | 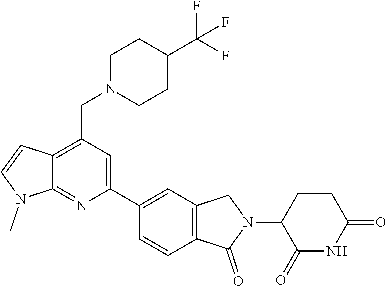 [SMILES: Cn1ccc2c(CN3CCC(C(F)(F)F)CC3)cc(-c3ccc4c(c3)CN(C3CCC(=O)NC3=O)C4=O)nc21] | 3-(5-(1-methyl-4-((4- (trifluoromethyl)piperidin-1- yl)methyl)-1H-pyrrolo[2,3- b]pyridin-6-yl)-1-oxoisoindolin-2- yl)piperidine-2,6-dione |
| 91 | 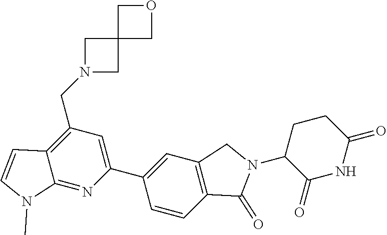 [SMILES: Cn1ccc2c(CN3CC4(COC4)C3)cc(-c3ccc4c(c3)CN(C3CCC(=O)NC3=O)C4=O)nc21] | 3-(5-(4-((2-oxa-6- azaspiro[3.3]heptan-6-yl)methyl)- 1-methyl-1H-pyrrolo[2,3- b]pyridin-6-yl)-1-oxoisoindolin-2- yl)piperidine-2,6-dione |
| 92 | 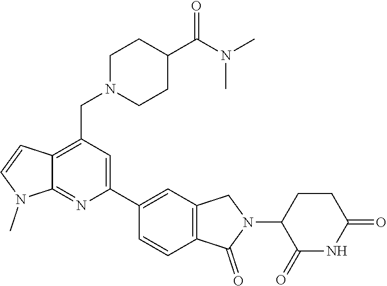 [SMILES: CN(C)C(=O)C1CCN(Cc2cc(-c3ccc4c(c3)CN(C3CCC(=O)NC3=O)C4=O)nc3c2ccn3C)CC1] | 1-((6-(2-(2,6-dioxopiperidin-3-yl)- 1-oxoisoindolin-5-yl)-1-methyl- 1H-pyrrolo[2,3-b]pyridin-4- yl)methyl)-N,N- dimethylpiperidine-4-carboxamide |
| 93 | 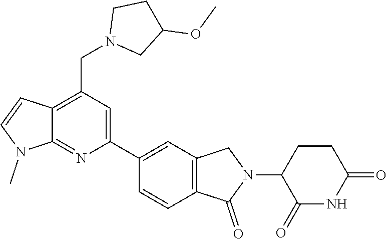 [SMILES: COC1CCN(Cc2cc(-c3ccc4c(c3)CN(C3CCC(=O)NC3=O)C4=O)nc3c2ccn3C)C1] | 3-(5-(4-((3-methoxypyrrolidin-1- yl)methyl)-1-methyl-1H- pyrrolo[2,3-b]pyridin-6-yl)-1- oxoisoindolin-2-yl)piperidine-2,6- dione |
| 94 | 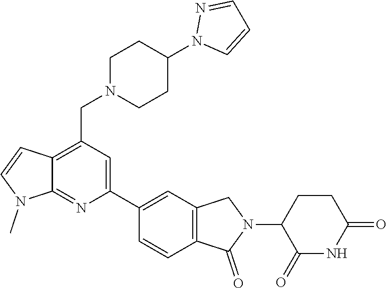 [SMILES: Cn1ccc2c(CN3CCC(n4cccn4)CC3)cc(-c3ccc4c(c3)CN(C3CCC(=O)NC3=O)C4=O)nc21] | 3-(5-(4-((4-(1H-pyrazol-1- yl)piperidin-1-yl)methyl)-1- methyl-1H-pyrrolo[2,3-b]pyridin- 6-yl)-1-oxoisoindolin-2- yl)piperidine-2,6-dione |
| 95 | 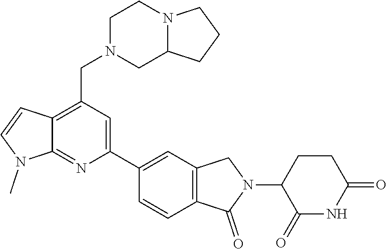 [SMILES: Cn1ccc2c(CN3CCN4CCCC4C3)cc(-c3ccc4c(c3)CN(C3CCC(=O)NC3=O)C4=O)nc21] | 3-(5-(4-((hexahydropyrrolo[1,2- a]pyrazin-2(1H)-yl)methyl)-1- methyl-1H-pyrrolo[2,3-b]pyridin- 6-yl)-1-oxoisoindolin-2- yl)piperidine-2,6-dione |
| 96 | 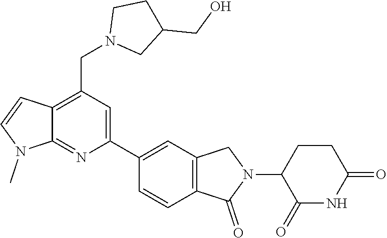 [SMILES: Cn1ccc2c(CN3CCC(CO)C3)cc(-c3ccc4c(c3)CN(C3CCC(=O)NC3=O)C4=O)nc21] | 3-(5-(4-((3- (hydroxymethyl)pyrrolidin-1- yl)methyl)-1-methyl-1H- pyrrolo[2,3-b]pyridin-6-yl)-1- oxoisoindolin-2-yl)piperidine-2,6- dione |
| 97 | 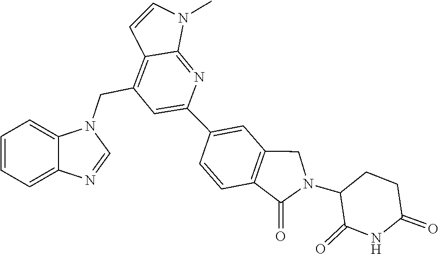 [SMILES: Cn1ccc2c(Cn3cnc4ccccc43)cc(-c3ccc4c(c3)CN(C3CCC(=O)NC3=O)C4=O)nc21] | 3-(5-(4-((1H-benzo[d]imidazol-1- yl)methyl)-1-methyl-1H- pyrrolo[2,3-b]pyridin-6-yl)-1- oxoisoindolin-2-yl)piperidine-2,6- dione |
| 98 | 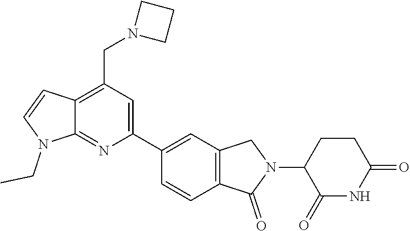 [SMILES: CCn1ccc2c(CN3CCC3)cc(-c3ccc4c(c3)CN(C3CCC(=O)NC3=O)C4=O)nc21] | 3-(5-(4-(azetidin-1-ylmethyl)-1- ethyl-1H-pyrrolo[2,3-b]pyridin-6- yl)-1-oxoisoindolin-2- yl)piperidine-2,6-dione |
| 99 | 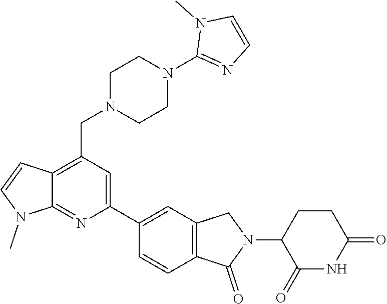 [SMILES: Cn1ccnc1N1CCN(Cc2cc(-c3ccc4c(c3)CN(C3CCC(=O)NC3=O)C4=O)nc3c2ccn3C)CC1] | 3-(5-(1-methyl-4-((4-(1-methyl- 1H-imidazol-2-yl)piperazin-1- yl)methyl)-1H-pyrrolo[2,3- b]pyridin-6-yl)-1-oxoisoindolin-2- yl)piperidine-2,6-dione |
| 100 | 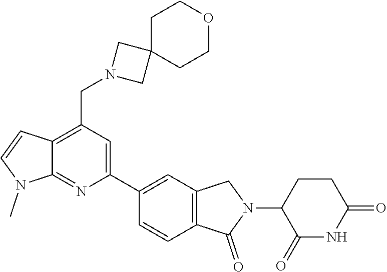 [SMILES: Cn1ccc2c(CN3CC4(CCOCC4)C3)cc(-c3ccc4c(c3)CN(C3CCC(=O)NC3=O)C4=O)nc21] | 3-(5-(4-((7-oxa-2- azaspiro[3.5]nonan-2-yl)methyl)-1- methyl-1H-pyrrolo[2,3-b]pyridin- 6-yl)-1-oxoisoindolin-2- yl)piperidine-2,6-dione |
| 101 | 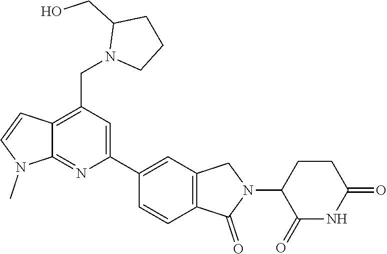 [SMILES: Cn1ccc2c(CN3CCCC3CO)cc(-c3ccc4c(c3)CN(C3CCC(=O)NC3=O)C4=O)nc21] | 3-(5-(4-((2- (hydroxymethyl)pyrrolidin-1- yl)methyl)-1-methyl-1H- pyrrolo[2,3-b]pyridin-6-yl)-1- oxoisoindolin-2-yl)piperidine-2,6- dione |
| 102 | 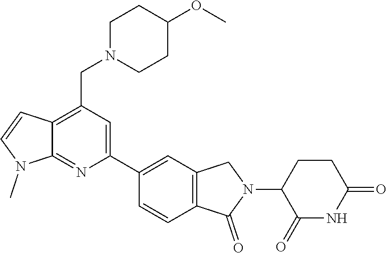 [SMILES: COC1CCN(Cc2cc(-c3ccc4c(c3)CN(C3CCC(=O)NC3=O)C4=O)nc3c2ccn3C)CC1] | 3-(5-(4-((4-methoxypiperidin-1- yl)methyl)-1-methyl-1H- pyrrolo[2,3-b]pyridin-6-yl)-1- oxoisoindolin-2-yl)piperidine-2,6- dione |
| 103 | 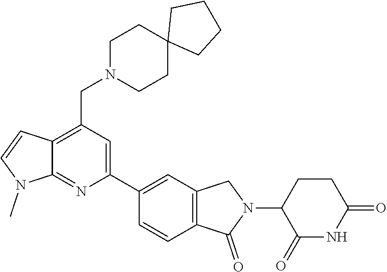 [SMILES: Cn1ccc2c(CN3CCC4(CCCC4)CC3)cc(-c3ccc4c(c3)CN(C3CCC(=O)NC3=O)C4=O)nc21] | 3-(5-(4-((8-azaspiro[4.5]decan-8- yl)methyl)-1-methyl-1H- pyrrolo[2,3-b]pyridin-6-yl)-1- oxoisoindolin-2-yl)piperidine-2,6- dione |
| 104 | 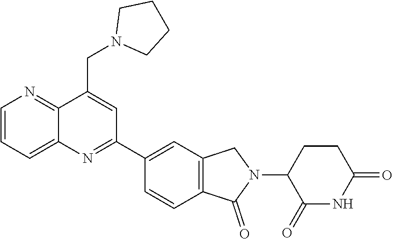 [SMILES: O=C1CCC(N2Cc3cc(-c4cc(CN5CCCC5)c5ncccc5n4)ccc3C2=O)C(=O)N1] | 3-(1-oxo-5-(4-(pyrrolidin-1- ylmethyl)-1,5-naphthyridin-2- yl)isoindolin-2-yl)piperidine-2,6- dione |
| 105 | 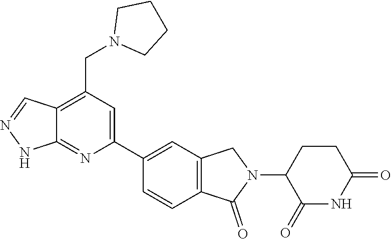 [SMILES: O=C1CCC(N2Cc3cc(-c4cc(CN5CCCC5)c5cn[nH]c5n4)ccc3C2=O)C(=O)N1] | 3-(1-oxo-5-(4-(pyrrolidin-1- ylmethyl)-1H-pyrazolo[3,4- b]pyridin-6-yl)isoindolin-2- yl)piperidine-2,6-dione |
| 106 | 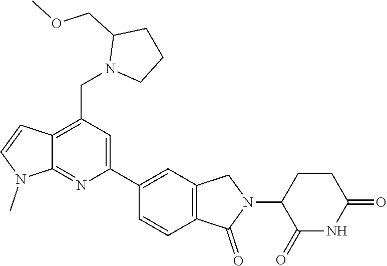 [SMILES: COCC1CCCN1Cc1cc(-c2ccc3c(c2)CN(C2CCC(=O)NC2=O)C3=O)nc2c1ccn2C] | 3-(5-(4-((2- (methoxymethyl)pyrrolidin-1- yl)methyl)-1-methyl-1H- pyrrolo[2,3-b]pyridin-6-yl)-1- oxoisoindolin-2-yl)piperidine-2,6- dione |
| 107 | 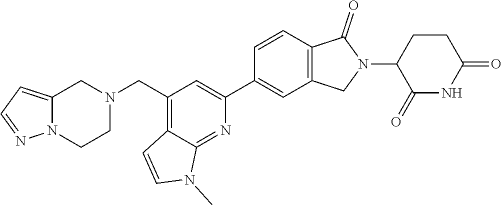 [SMILES: Cn1ccc2c(CN3CCn4nccc4C3)cc(-c3ccc4c(c3)CN(C3CCC(=O)NC3=O)C4=O)nc21] | 3-(5-(4-((6,7-dihydropyrazolo[1,5- a]pyrazin-5(4H)-yl)methyl)-1- methyl-1H-pyrrolo[2,3-b]pyridin- 6-yl)-1-oxoisoindolin-2- yl)piperidine-2,6-dione |
| 108 | 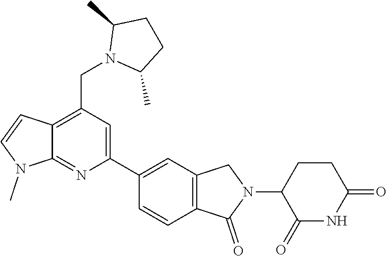 [SMILES: C[C@H]1CC[C@H](C)N1Cc1cc(-c2ccc3c(c2)CN(C2CCC(=O)NC2=O)C3=O)nc2c1ccn2C] | 3-(5-(4-(((2S,5S)-2,5- dimethylpyrrolidin-1-yl)methyl)-1- methyl-1H-pyrrolo[2,3-b]pyridin- 6-yl)-1-oxoisoindolin-2- yl)piperidine-2,6-dione |
| 109 | 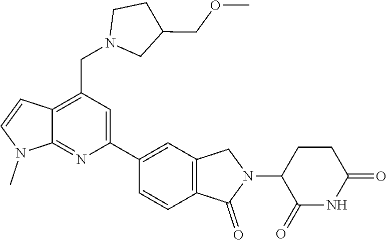 [SMILES: COCC1CCN(Cc2cc(-c3ccc4c(c3)CN(C3CCC(=O)NC3=O)C4=O)nc3c2ccn3C)C1] | 3-(5-(4-((3- (methoxymethyl)pyrrolidin-1- yl)methyl)-1-methyl-1H- pyrrolo[2,3-b]pyridin-6-yl)-1- oxoisoindolin-2-yl)piperidine-2,6- dione |
| 110 | 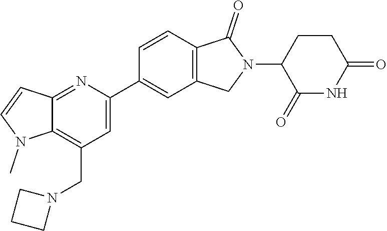 [SMILES: Cn1ccc2nc(-c3ccc4c(c3)CN(C3CCC(=O)NC3=O)C4=O)cc(CN3CCC3)c21] | 3-(5-(7-(azetidin-1-ylmethyl)-1- methyl-1H-pyrrolo[3,2-b]pyridin- 5-yl)-1-oxoisoindolin-2- yl)piperidine-2,6-dione |
| 111 | 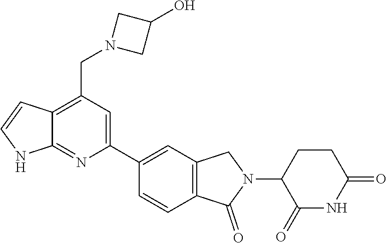 [SMILES: O=C1CCC(N2Cc3cc(-c4cc(CN5CC(O)C5)c5cc[nH]c5n4)ccc3C2=O)C(=O)N1] | 3-(5-(4-((3-hydroxyazetidin-1- yl)methyl)-1H-pyrrolo[2,3- b]pyridin-6-yl)-1-oxoisoindolin-2- yl)piperidine-2,6-dione |
| 112 | 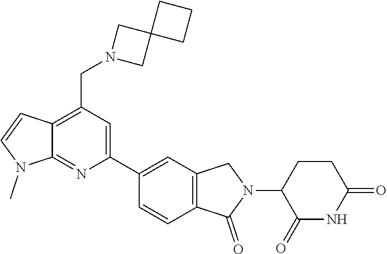 [SMILES: Cn1ccc2c(CN3CC4(CCC4)C3)cc(-c3ccc4c(c3)CN(C3CCC(=O)NC3=O)C4=O)nc21] | 3-(5-(4-((2-azaspiro[3.3]heptan-2- yl)methyl)-1-methyl-1H- pyrrolo[2,3-b]pyridin-6-yl)-1- oxoisoindolin-2-yl)piperidine-2,6- dione |
| 113 | 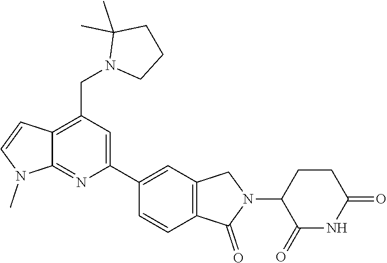 [SMILES: Cn1ccc2c(CN3CCCC3(C)C)cc(-c3ccc4c(c3)CN(C3CCC(=O)NC3=O)C4=O)nc21] | 3-(5-(4-((2,2-dimethylpyrrolidin-1- yl)methyl)-1-methyl-1H- pyrrolo[2,3-b]pyridin-6-yl)-1- oxoisoindolin-2-yl)piperidine-2,6- dione |
| 114 | 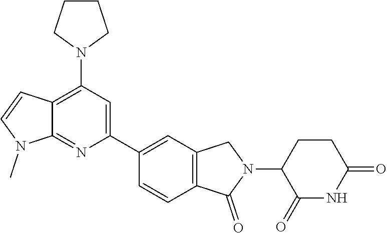 [SMILES: Cn1ccc2c(N3CCCC3)cc(-c3ccc4c(c3)CN(C3CCC(=O)NC3=O)C4=O)nc21] | 3-(5-(1-methyl-4-(pyrrolidin-1-yl)- 1H-pyrrolo[2,3-b]pyridin-6-yl)-1- oxoisoindolin-2-yl)piperidine-2,6- dione |
| 115 | 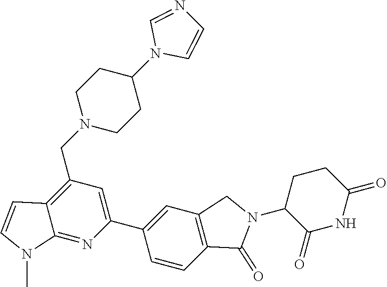 [SMILES: Cn1ccc2c(CN3CCC(n4ccnc4)CC3)cc(-c3ccc4c(c3)CN(C3CCC(=O)NC3=O)C4=O)nc21] | 3-(5-(4-((4-(1H-imidazol-1- yl)piperidin-1-yl)methyl)-1- methyl-1H-pyrrolo[2,3-b]pyridin- 6-yl)-1-oxoisoindolin-2- yl)piperidine-2,6-dione |
| 116 | 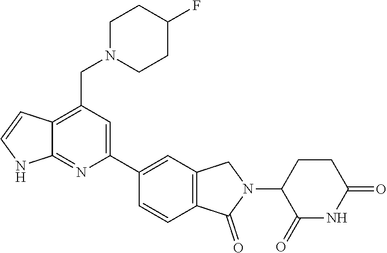 [SMILES: O=C1CCC(N2Cc3cc(-c4cc(CN5CCC(F)CC5)c5cc[nH]c5n4)ccc3C2=O)C(=O)N1] | 3-(5-(4-((4-fluoropiperidin-1- yl)methyl)-1H-pyrrolo[2,3- b]pyridin-6-yl)-1-oxoisoindolin-2- yl)piperidine-2,6-dione |
| 117 | 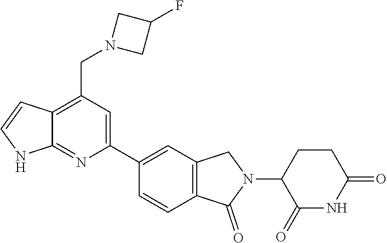 [SMILES: O=C1CCC(N2Cc3cc(-c4cc(CN5CC(F)C5)c5cc[nH]c5n4)ccc3C2=O)C(=O)N1] | 3-(5-(4-((3-fluoroazetidin-1- yl)methyl)-1H-pyrrolo[2,3- b]pyridin-6-yl)-1-oxoisoindolin-2- yl)piperidine-2,6-dione |
| 118 | 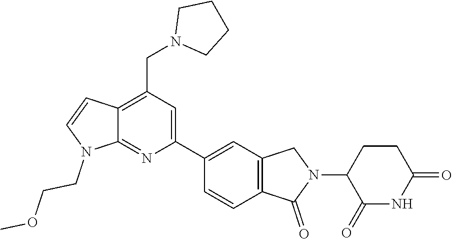 [SMILES: COCCn1ccc2c(CN3CCCC3)cc(-c3ccc4c(c3)CN(C3CCC(=O)NC3=O)C4=O)nc21] | 3-(5-(1-(2-methoxyethyl)-4- (pyrrolidin-1-ylmethyl)-1H- pyrrolo[2,3-b]pyridin-6-yl)-1- oxoisoindolin-2-yl)piperidine-2,6- dione |
| 119 | 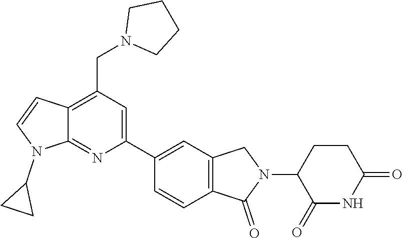 [SMILES: O=C1CCC(N2Cc3cc(-c4cc(CN5CCCC5)c5ccn(C6CC6)c5n4)ccc3C2=O)C(=O)N1] | 3-(5-(1-cyclopropyl-4-(pyrrolidin- 1-ylmethyl)-1H-pyrrolo[2,3- b]pyridin-6-yl)-1-oxoisoindolin-2- yl)piperidine-2,6-dione |
| 120 | 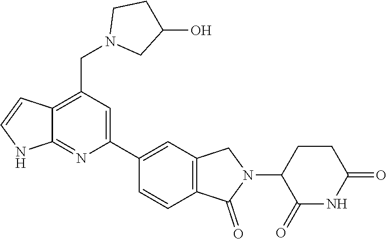 [SMILES: O=C1CCC(N2Cc3cc(-c4cc(CN5CCC(O)C5)c5cc[nH]c5n4)ccc3C2=O)C(=O)N1] | 3-(5-(4-((3-hydroxypyrrolidin-1- yl)methyl)-1H-pyrrolo[2,3- b]pyridin-6-yl)-1-oxoisoindolin-2- yl)piperidine-2,6-dione |
| 121 | 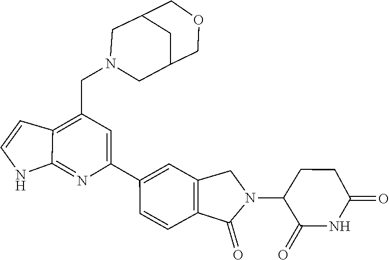 [SMILES: O=C1CCC(N2Cc3cc(-c4cc(CN5CC6COCC(C6)C5)c5cc[nH]c5n4)ccc3C2=O)C(=O)N1] | 3-(5-(4-((3-oxa-7- azabicyclo[3.3.1]nonan-7- yl)methyl)-1H-pyrrolo[2,3- b]pyridin-6-yl)-1-oxoisoindolin-2- yl)piperidine-2,6-dione |
| 122 | 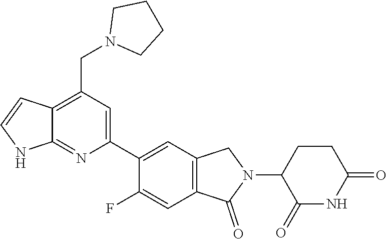 [SMILES: O=C1CCC(N2Cc3cc(-c4cc(CN5CCCC5)c5cc[nH]c5n4)c(F)cc3C2=O)C(=O)N1] | 3-(6-fluoro-1-oxo-5-(4-(pyrrolidin- 1-ylmethyl)-1H-pyrrolo[2,3- b]pyridin-6-yl)isoindolin-2- yl)piperidine-2,6-dione |
| 123 | 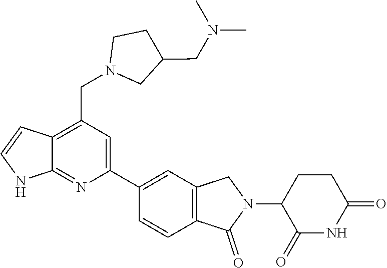 [SMILES: CN(C)CC1CCN(Cc2cc(-c3ccc4c(c3)CN(C3CCC(=O)NC3=O)C4=O)nc3[nH]ccc23)C1] | 3-(5-(4-((3-((dimethyl- amino)methyl)pyrrolidin- 1-yl)methyl)-1H-pyrrolo[2,3- b]pyridin-6-yl)-1-oxoisoindolin-2- yl)piperidine-2,6-dione |
| 124 | 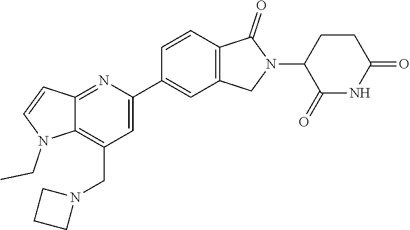 [SMILES: CCn1ccc2nc(-c3ccc4c(c3)CN(C3CCC(=O)NC3=O)C4=O)cc(CN3CCC3)c21] | 3-(5-(7-(azetidin-1-ylmethyl)-1- ethyl-1H-pyrrolo[3,2-b]pyridin-5- yl)-1-oxoisoindolin-2- yl)piperidine-2,6-dione |
| 125 | 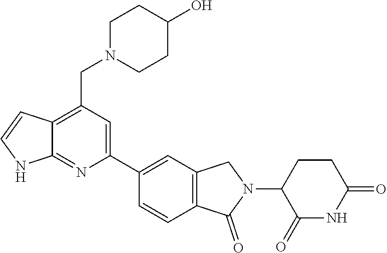 [SMILES: O=C1CCC(N2Cc3cc(-c4cc(CN5CCC(O)CC5)c5cc[nH]c5n4)ccc3C2=O)C(=O)N1] | 3-(5-(4-((4-hydroxypiperidin-1- yl)methyl)-1H-pyrrolo[2,3- b]pyridin-6-yl)-1-oxoisoindolin-2- yl)piperidine-2,6-dione |
| 126 | 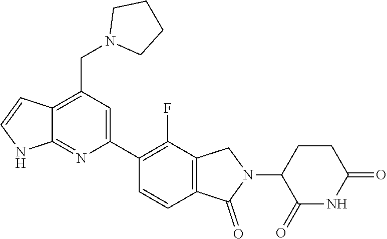 [SMILES: O=C1CCC(N2Cc3c(ccc(-c4cc(CN5CCCC5)c5cc[nH]c5n4)c3F)C2=O)C(=O)N1] | 3-(4-fluoro-1-oxo-5-(4-(pyrrolidin- 1-ylmethyl)-1H-pyrrolo[2,3- b]pyridin-6-yl)isoindolin-2- yl)piperidine-2,6-dione |
| 127 | 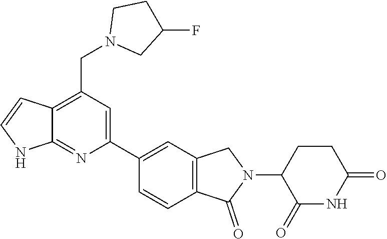 [SMILES: O=C1CCC(N2Cc3cc(-c4cc(CN5CCC(F)C5)c5cc[nH]c5n4)ccc3C2=O)C(=O)N1] | 3-(5-(4-((3-fluoropyrrolidin-1- yl)methyl)-1H-pyrrolo[2,3- b]pyridin-6-yl)-1-oxoisoindolin-2- yl)piperidine-2,6-dione |
| 128 | 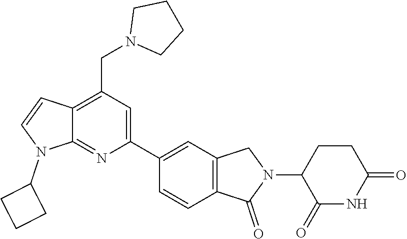 [SMILES: O=C1CCC(N2Cc3cc(-c4cc(CN5CCCC5)c5ccn(C6CCC6)c5n4)ccc3C2=O)C(=O)N1] | 3-(5-(1-cyclobutyl-4-(pyrrolidin-1- ylmethyl)-1H-pyrrolo[2,3- b]pyridin-6-yl)-1-oxoisoindolin-2- yl)piperidine-2,6-dione |
| 129 | 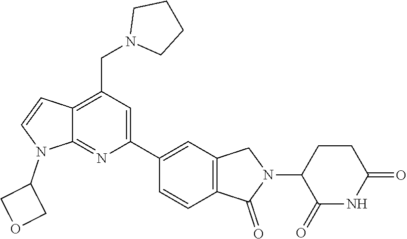 [SMILES: O=C1CCC(N2Cc3cc(-c4cc(CN5CCCC5)c5ccn(C6COC6)c5n4)ccc3C2=O)C(=O)N1] | 3-(5-(1-(oxetan-3-y1)-4-(pyrrolidin- 1-ylmethyl)-1H-pyrrolo[2,3- b]pyridin-6-yl)-1-oxoisoindolin-2- yl)piperidine-2,6-dione |
| 130 | 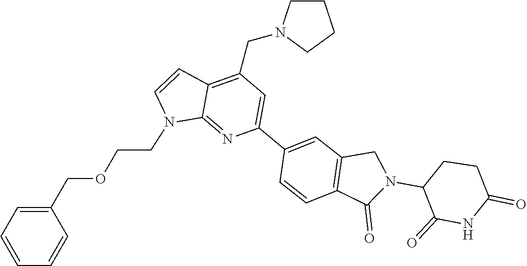 [SMILES: O=C1CCC(N2Cc3cc(-c4cc(CN5CCCC5)c5ccn(CCOCc6ccccc6)c5n4)ccc3C2=O)C(=O)N1] | 3-(5-(1-(2-(benzyloxy)ethyl)-4- (pyrrolidin-1-ylmethyl)-1H- pyrrolo[2,3-b]pyridin-6-yl)-1- oxoisoindolin-2-yl)piperidine-2,6- dione |
| 131 | 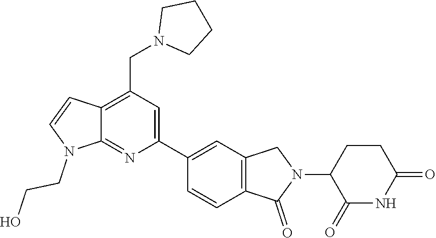 [SMILES: O=C1CCC(N2Cc3cc(-c4cc(CN5CCCC5)c5ccn(CCO)c5n4)ccc3C2=O)C(=O)N1] | 3-(5-(1-(2-hydroxyethyl)-4- (pyrrolidin-1-ylmethyl)-1H- pyrrolo[2,3-b]pyridin-6-yl)-1- oxoisoindolin-2-yl)piperidine-2,6- dione |
| 133 | 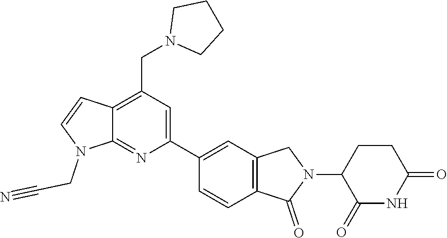 [SMILES: N#CCn1ccc2c(CN3CCCC3)cc(-c3ccc4c(c3)CN(C3CCC(=O)NC3=O)C4=O)nc21] | 2-(6-(2-(2,6-dioxopiperidin-3-yl)- 1-oxoisoindolin-5-yl)-4- (pyrrolidin-1-ylmethyl)-1H- pyrrolo[2,3-b]pyridin-1- yl)acetonitrile |
| 134 | 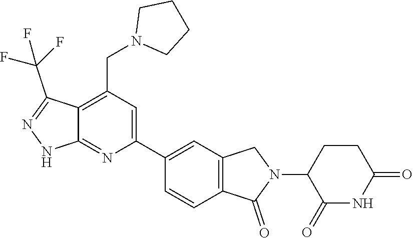 [SMILES: O=C1CCC(N2Cc3cc(-c4cc(CN5CCCC5)c5c(C(F)(F)F)n[nH]c5n4)ccc3C2=O)C(=O)N1] | 3-(1-oxo-5-(4-(pyrrolidin-1- ylmethyl)-3-(trifluoromethyl)-1H- pyrazolo[3,4-b]pyridin-6- yl)isoindolin-2-yl)piperidine-2,6- dione |
| 135 | 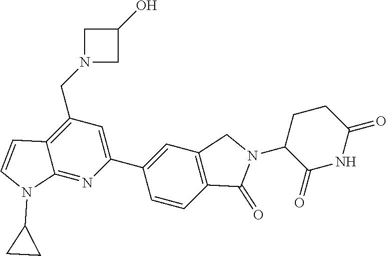 [SMILES: O=C1CCC(N2Cc3cc(-c4cc(CN5CC(O)C5)c5ccn(C6CC6)c5n4)ccc3C2=O)C(=O)N1] | 3-(5-(1-cyclopropyl-4-((3- hydroxyazetidin-1-yl)methyl)-1H- pyrrolo[2,3-b]pyridin-6-yl)-1- oxoisoindolin-2-yl)piperidine-2,6- dione |
| 136 | 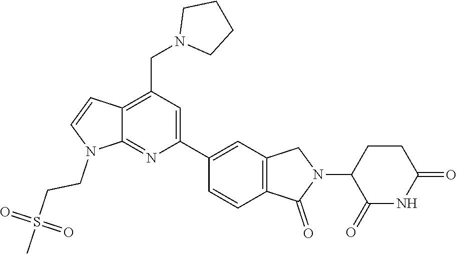 [SMILES: CS(=O)(=O)CCn1ccc2c(CN3CCCC3)cc(-c3ccc4c(c3)CN(C3CCC(=O)NC3=O)C4=O)nc21] | 3-(5-(1-(2-(methylsulfonyl)ethyl)- 4-(pyrrolidin-1-ylmethyl)-1H- pyrrolo[2,3-b]pyridin-6-yl)-1- oxoisoindolin-2-yl)piperidine-2,6- dione |
| 137 | 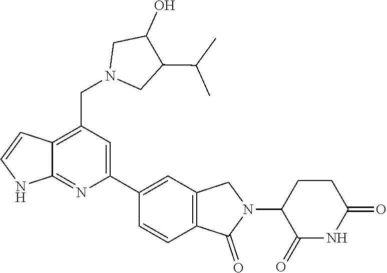 [SMILES: CC(C)C1CN(Cc2cc(-c3ccc4c(c3)CN(C3CCC(=O)NC3=O)C4=O)nc3[nH]ccc23)CC1O] | 3-(5-(4-((3-hydroxy-4- isopropylpyrrolidin-1-yl)methyl)- 1H-pyrrolo[2,3-b]pyridin-6-yl)-1- oxoisoindolin-2-yl)piperidine-2,6- dione |
| 138 | 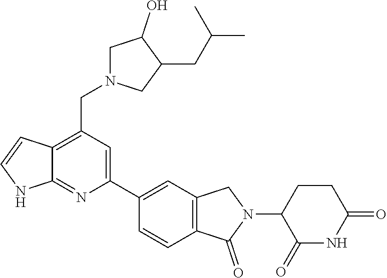 [SMILES: CC(C)CC1CN(Cc2cc(-c3ccc4c(c3)CN(C3CCC(=O)NC3=O)C4=O)nc3[nH]ccc23)CC1O] | 3-(5-(4-((3-hydroxy-4- isobutylpyrrolidin-1-yl)methyl)- 1H-pyrrolo[2,3-b]pyridin-6-yl)-1- oxoisoindolin-2-yl)piperidine-2,6- dione |
| 139 | 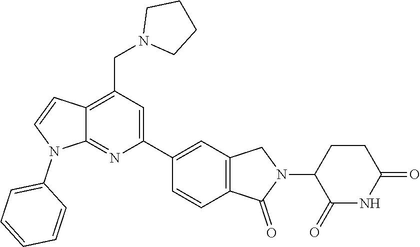 [SMILES: O=C1CCC(N2Cc3cc(-c4cc(CN5CCCC5)c5ccn(-c6ccccc6)c5n4)ccc3C2=O)C(=O)N1] | 3-(1-oxo-5-(1-phenyl-4- (pyrrolidin-1-ylmethyl)-1H- pyrrolo[2,3-b]pyridin-6- yl)isoindolin-2-yl)piperidine-2,6- dione |
| 140 | 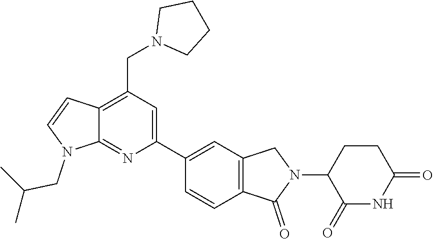 [SMILES: CC(C)Cn1ccc2c(CN3CCCC3)cc(-c3ccc4c(c3)CN(C3CCC(=O)NC3=O)C4=O)nc21] | 3-(5-(1-isobutyl-4-(pyrrolidin-1- ylmethyl)-1H-pyrrolo[2,3- b]pyridin-6-yl)-1-oxoisoindolin-2- yl)piperidine-2,6-dione |
| 141 | 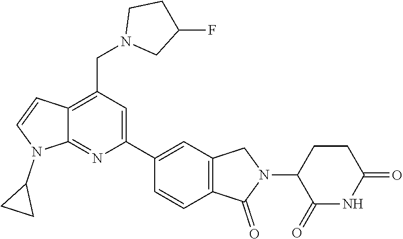 [SMILES: O=C1CCC(N2Cc3cc(-c4cc(CN5CCC(F)C5)c5ccn(C6CC6)c5n4)ccc3C2=O)C(=O)N1] | 3-(5-(1-cyclopropyl-4-((3- fluoropyrrolidin-1-yl)methyl)-1H- pyrrolo[2,3-b]pyridin-6-yl)-1- oxoisoindolin-2-yl)piperidine-2,6- dione |
| 142 | 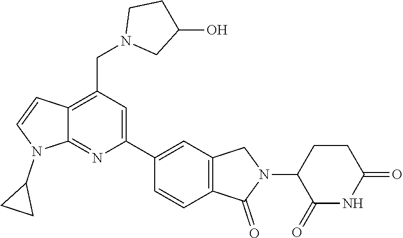 [SMILES: O=C1CCC(N2Cc3cc(-c4cc(CN5CCC(O)C5)c5ccn(C6CC6)c5n4)ccc3C2=O)C(=O)N1] | 3-(5-(1-cyclopropyl-4-((3- hydroxypyrrolidin-1-yl)methyl)- 1H-pyrrolo[2,3-b]pyridin-6-yl)-1- oxoisoindolin-2-yl)piperidine-2,6- dione |
| 143 | 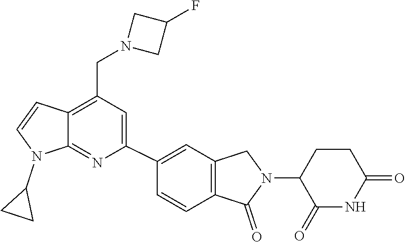 [SMILES: O=C1CCC(N2Cc3cc(-c4cc(CN5CC(F)C5)c5ccn(C6CC6)c5n4)ccc3C2=O)C(=O)N1] | 3-(5-(1-cyclopropyl-4-((3- fluoroazetidin-1-yl)methyl)-1H- pyrrolo[2,3-b]pyridin-6-yl)-1- oxoisoindolin-2-yl)piperidine-2,6- dione |
| 144 | 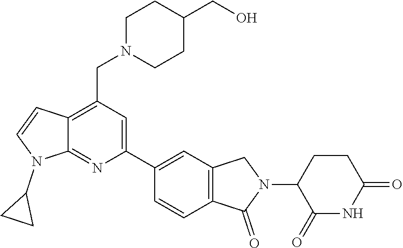 [SMILES: O=C1CCC(N2Cc3cc(-c4cc(CN5CCC(CO)CC5)c5ccn(C6CC6)c5n4)ccc3C2=O)C(=O)N1] | 3-(5-(1-cyclopropyl-4-((4- (hydroxymethyl)piperidin-1- yl)methyl)-1H-pyrrolo[2,3- b]pyridin-6-yl)-1-oxoisoindolin-2- yl)piperidine-2,6-dione |
| 145 | 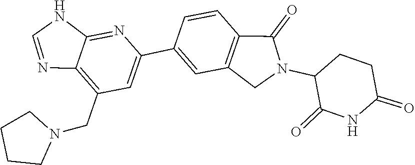 [SMILES: O=C1CCC(N2Cc3cc(-c4cc(CN5CCCC5)c5nc[nH]c5n4)ccc3C2=O)C(=O)N1] | 3-(1-oxo-5-(7-(pyrrolidin-1- ylmethyl)-3H-imidazo[4,5- b]pyridin-5-yl)isoindolin-2- yl)piperidine-2,6-dione |
| 146 | 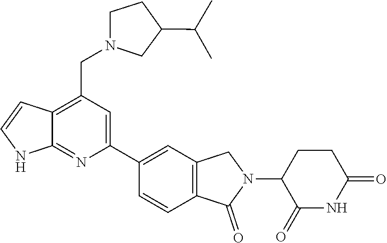 [SMILES: CC(C)C1CCN(Cc2cc(-c3ccc4c(c3)CN(C3CCC(=O)NC3=O)C4=O)nc3[nH]ccc23)C1] | 3-(5-(4-((3-isopropylpyrrolidin-1- yl)methyl)-1H-pyrrolo[2,3- b]pyridin-6-yl)-1-oxoisoindolin-2- yl)piperidine-2,6-dione |
| 147 | 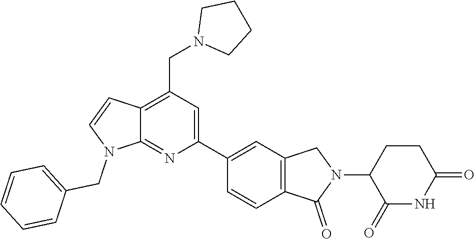 [SMILES: O=C1CCC(N2Cc3cc(-c4cc(CN5CCCC5)c5ccn(Cc6ccccc6)c5n4)ccc3C2=O)C(=O)N1] | 3-(5-(1-benzyl-4-(pyrrolidin-1- ylmethyl)-1H-pyrrolo[2,3- b]pyridin-6-yl)-1-oxoisoindolin-2- yl)piperidine-2,6-dione |
| 148 | 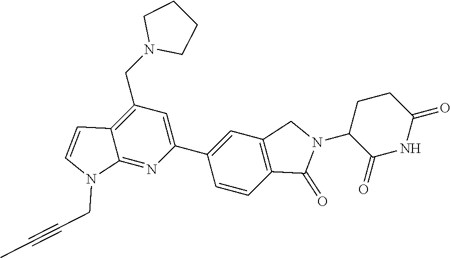 [SMILES: CC#CCn1ccc2c(CN3CCCC3)cc(-c3ccc4c(c3)CN(C3CCC(=O)NC3=O)C4=O)nc21] | 3-(5-(1-(but-2-yn-1-yl)-4- (pyrrolidin-1-ylmethyl)-1H- pyrrolo[2,3-b]pyridin-6-yl)-1- oxoisoindolin-2-yl)piperidine-2,6- dione |
| 149 | 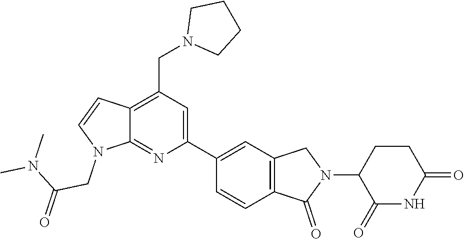 [SMILES: CN(C)C(=O)Cn1ccc2c(CN3CCCC3)cc(-c3ccc4c(c3)CN(C3CCC(=O)NC3=O)C4=O)nc21] | 2-(6-(2-(2,6-dioxopiperidin-3-yl)- 1-oxoisoindolin-5-yl)-4- (pyrrolidin-1-ylmethyl)-1H- pyrrolo[2,3-b]pyridin-1-yl)-N,N- dimethylacetamide |
| 150 | 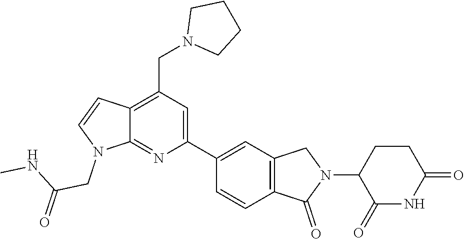 [SMILES: CNC(=O)Cn1ccc2c(CN3CCCC3)cc(-c3ccc4c(c3)CN(C3CCC(=O)NC3=O)C4=O)nc21] | 2-(6-(2-(2,6-dioxopiperidin-3-yl)- 1-oxoisoindolin-5-yl)-4- (pyrrolidin-1-ylmethyl)-1H- pyrrolo[2,3-b]pyridin-1-yl)-N- methylacetamide |
| 151 | 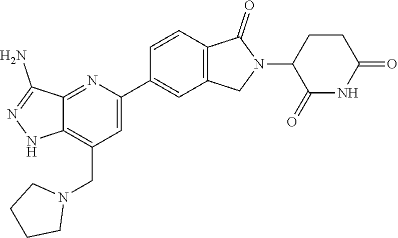 [SMILES: Nc1n[nH]c2c(CN3CCCC3)cc(-c3ccc4c(c3)CN(C3CCC(=O)NC3=O)C4=O)nc12] | 3-(5-(3-amino-7-(pyrrolidin-1- ylmethyl)-1H-pyrazolo[4,3- b]pyridin-5-yl)-1-oxoisoindolin-2- yl)piperidine-2,6-dione |
| 152 | 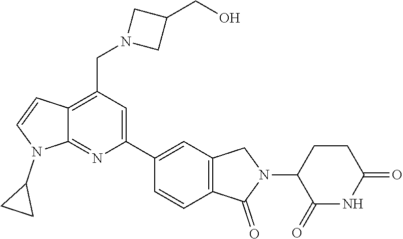 [SMILES: O=C1CCC(N2Cc3cc(-c4cc(CN5CC(CO)C5)c5ccn(C6CC6)c5n4)ccc3C2=O)C(=O)N1] | 3-(5-(1-cyclopropyl-4-((3- (hydroxymethyl)azetidin-1- yl)methyl)-1H-pyrrolo[2,3- b]pyridin-6-yl)-1-oxoisoindolin-2- yl)piperidine-2,6-dione |
| 153 | 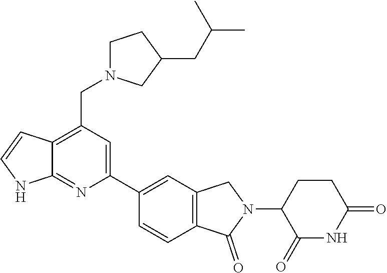 [SMILES: CC(C)CC1CCN(Cc2cc(-c3ccc4c(c3)CN(C3CCC(=O)NC3=O)C4=O)nc3[nH]ccc23)C1] | 3-(5-(4-((3-isobutylpyrrolidin-1- yl)methyl)-1H-pyrrolo[2,3- b]pyridin-6-yl)-1-oxoisoindolin-2- yl)piperidine-2,6-dione |
| 154 | 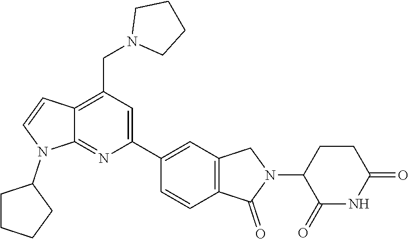 [SMILES: O=C1CCC(N2Cc3cc(-c4cc(CN5CCCC5)c5ccn(C6CCCC6)c5n4)ccc3C2=O)C(=O)N1] | 3-(5-(1-cyclopentyl-4-(pyrrolidin- 1-ylmethyl)-1H-pyrrolo[2,3- b]pyridin-6-yl)-1-oxoisoindolin-2- yl)piperidine-2,6-dione |
| 155 | 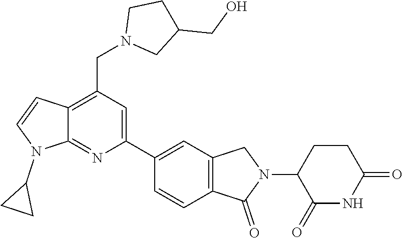 [SMILES: O=C1CCC(N2Cc3cc(-c4cc(CN5CCC(CO)C5)c5ccn(C6CC6)c5n4)ccc3C2=O)C(=O)N1] | 3-(5-(1-cyclopropyl-4-((3- (hydroxymethyl)pyrrolidin-1- yl)methyl)-1H-pyrrolo[2,3- b]pyridin-6-yl)-1-oxoisoindolin-2- yl)piperidine-2,6-dione |
| 156 | 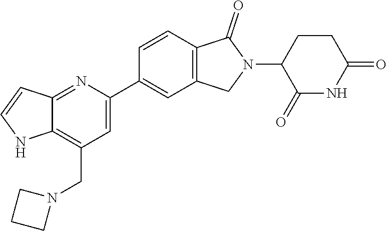 [SMILES: O=C1CCC(N2Cc3cc(-c4cc(CN5CCC5)c5[nH]ccc5n4)ccc3C2=O)C(=O)N1] | 3-(5-(7-(azetidin-1-ylmethyl)-1H- pyrrolo[3,2-b]pyridin-5-yl)-1- oxoisoindolin-2-yl)piperidine-2,6- dione |
| 157 | 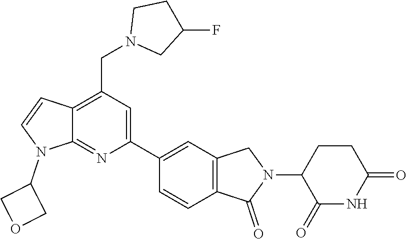 [SMILES: O=C1CCC(N2Cc3cc(-c4cc(CN5CCC(F)C5)c5ccn(C6COC6)c5n4)ccc3C2=O)C(=O)N1] | 3-(5-(4-((3-fluoropyrrolidin-1- yl)methyl)-1-(oxetan-3-yl)-1H- pyrrolo[2,3-b]pyridin-6-yl)-1- oxoisoindolin-2-yl)piperidine-2,6- dione |
| 158 | 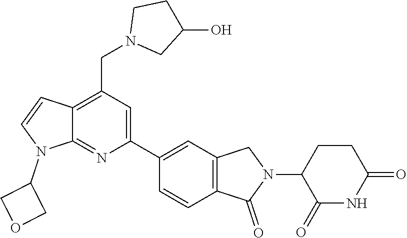 [SMILES: O=C1CCC(N2Cc3cc(-c4cc(CN5CCC(O)C5)c5ccn(C6COC6)c5n4)ccc3C2=O)C(=O)N1] | 3-(5-(4-((3-hydroxypyrrolidin-1- yl)methyl)-1-(oxetan-3-yl)-1H- pyrrolo[2,3-b]pyridin-6-yl)-1- oxoisoindolin-2-yl)piperidine-2,6- dione |
| 159 | 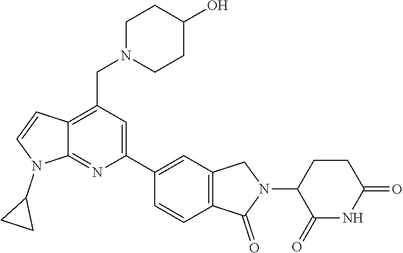 [SMILES: O=C1CCC(N2Cc3cc(-c4cc(CN5CCC(O)CC5)c5ccn(C6CC6)c5n4)ccc3C2=O)C(=O)N1] | 3-(5-(1-cyclopropyl-4-((4- hydroxypiperidin-1-yl)methyl)-1H- pyrrolo[2,3-b]pyridin-6-yl)-1- oxoisoindolin-2-yl)piperidine-2,6- dione |
| 160 | 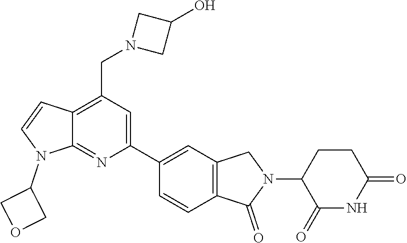 [SMILES: O=C1CCC(N2Cc3cc(-c4cc(CN5CC(O)C5)c5ccn(C6COC6)c5n4)ccc3C2=O)C(=O)N1] | 3-(5-(4-((3-hydroxyazetidin-1- yl)methyl)-1-(oxetan-3-yl)-1H- pyrrolo[2,3-b]pyridin-6-yl)-1- oxoisoindolin-2-yl)piperidine-2,6- dione |
| 161 | 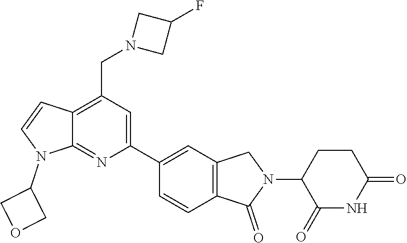 [SMILES: O=C1CCC(N2Cc3cc(-c4cc(CN5CC(F)C5)c5ccn(C6COC6)c5n4)ccc3C2=O)C(=O)N1] | 3-(5-(4-((3-fluoroazetidin-1- yl)methyl)-1-(oxetan-3-yl)-1H- pyrrolo[2,3-b]pyridin-6-yl)-1- oxoisoindolin-2-yl)piperidine-2,6- dione |
| 162 | 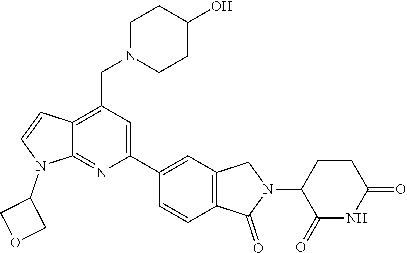 [SMILES: O=C1CCC(N2Cc3cc(-c4cc(CN5CCC(O)CC5)c5ccn(C6COC6)c5n4)ccc3C2=O)C(=O)N1] | 3-(5-(4-((4-hydroxypiperidin-1- yl)methyl)-1-(oxetan-3-yl)-1H- pyrrolo[2,3-b]pyridin-6-yl)-1- oxoisoindolin-2-yl)piperidine-2,6- dione |
| 163 | 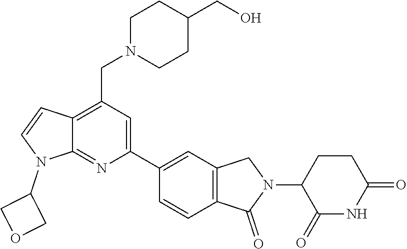 [SMILES: O=C1CCC(N2Cc3cc(-c4cc(CN5CCC(CO)CC5)c5ccn(C6COC6)c5n4)ccc3C2=O)C(=O)N1] | 3-(5-(4-((4- (hydroxymethyl)piperidin-1- yl)methyl)-1-(oxetan-3-yl)-1H- pyrrolo[2,3-b]pyridin-6-yl)-1- oxoisoindolin-2-yl)piperidine-2,6- dione |
| 164 | 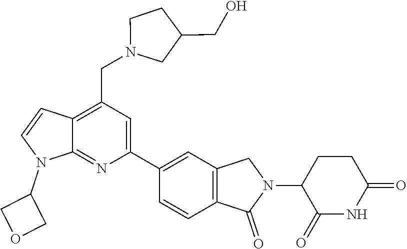 [SMILES: O=C1CCC(N2Cc3cc(-c4cc(CN5CCC(CO)C5)c5ccn(C6COC6)c5n4)ccc3C2=O)C(=O)N1] | 3-(5-(4-((3- (hydroxymethyl)pyrrolidin-1- yl)methyl)-1-(oxetan-3-yl)-1H- pyrrolo[2,3-b]pyridin-6-yl)-1- oxoisoindolin-2-yl)piperidine-2,6- dione |
| 165 | 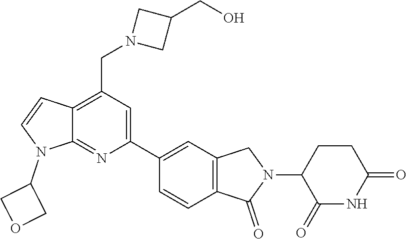 [SMILES: O=C1CCC(N2Cc3cc(-c4cc(CN5CC(CO)C5)c5ccn(C6COC6)c5n4)ccc3C2=O)C(=O)N1] | 3-(5-(4-((3- (hydroxymethyl)azetidin-1- yl)methyl)-1-(oxetan-3-yl)-1H- pyrrolo[2,3-b]pyridin-6-yl)-1- oxoisoindolin-2-yl)piperidine-2,6- dione |
| 166 | 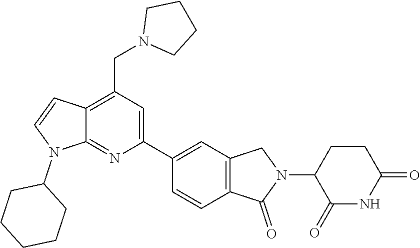 [SMILES: O=C1CCC(N2Cc3cc(-c4cc(CN5CCCC5)c5ccn(C6CCCCC6)c5n4)ccc3C2=O)C(=O)N1] | 3-(5-(1-cyclohexyl-4-(pyrrolidin-1- ylmethyl)-1H-pyrrolo[2,3- b]pyridin-6-yl)-1-oxoisoindolin-2- yl)piperidine-2,6-dione |
| 167 | 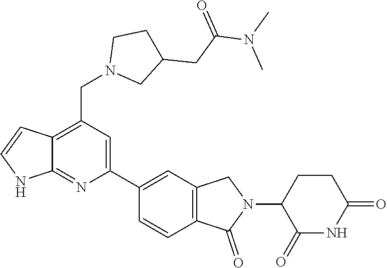 [SMILES: CN(C)C(=O)CC1CCN(Cc2cc(-c3ccc4c(c3)CN(C3CCC(=O)NC3=O)C4=O)nc3[nH]ccc23)C1] | 2-(1-((6-(2-(2,6-dioxopiperidin-3- yl)-1-oxoisoindolin-5-yl)-1H- pyrrolo[2,3-b]pyridin-4- yl)methyl)pyrrolidin-3-yl)-N,N- dimethylacetamide |
| 168 | 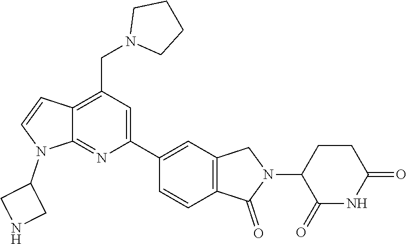 [SMILES: O=C1CCC(N2Cc3cc(-c4cc(CN5CCCC5)c5ccn(C6CNC6)c5n4)ccc3C2=O)C(=O)N1] | 3-(5-(1-(azetidin-3-yl)-4- (pyrrolidin-1-ylmethyl)-1H- pyrrolo[2,3-b]pyridin-6-yl)-1- oxoisoindolin-2-yl)piperidine-2,6- dione |
| 169 | 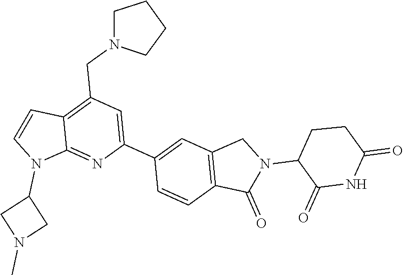 [SMILES: CN1CC(n2ccc3c(CN4CCCC4)cc(-c4ccc5c(c4)CN(C4CCC(=O)NC4=O)C5=O)nc32)C1] | 3-(5-(1-(1-methylazetidin-3-yl)-4- (pyrrolidin-1-ylmethyl)-1H- pyrrolo[2,3-b]pyridin-6-yl)-1- oxoisoindolin-2-yl)piperidine-2,6- dione |
| 170 | 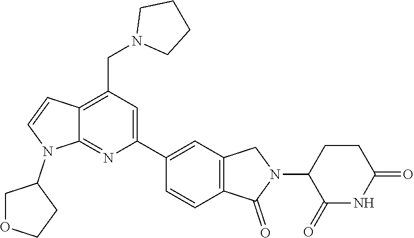 [SMILES: O=C1CCC(N2Cc3cc(-c4cc(CN5CCCC5)c5ccn(C6CCOC6)c5n4)ccc3C2=O)C(=O)N1] | 3-(1-oxo-5-(4-(pyrrolidin-1- ylmethyl)-1-(tetrahydrofuran-3-yl)- 1H-pyrrolo[2,3-b]pyridin-6- yl)isoindolin-2-yl)piperidine-2,6- dione |
| 171 | 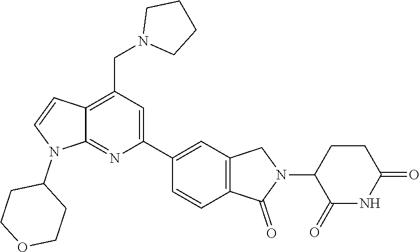 [SMILES: O=C1CCC(N2Cc3cc(-c4cc(CN5CCCC5)c5ccn(C6CCOCC6)c5n4)ccc3C2=O)C(=O)N1] | 3-(1-oxo-5-(4-(pyrrolidin-1- ylmethyl)-1-(tetrahydro-2H-pyran- 4-yl)-1H-pyrrolo[2,3-b]pyridin-6- yl)isoindolin-2-yl)piperidine-2,6- dione |
| 172 | 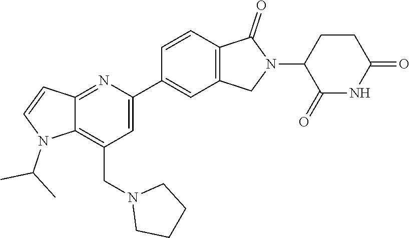 [SMILES: CC(C)n1ccc2nc(-c3ccc4c(c3)CN(C3CCC(=O)NC3=O)C4=O)cc(CN3CCCC3)c21] | 3-(5-(1-isopropyl-7-(pyrrolidin-1- ylmethyl)-1H-pyrrolo[3,2- b]pyridin-5-yl)-1-oxoisoindolin-2- yl)piperidine-2,6-dione |
| 173 | 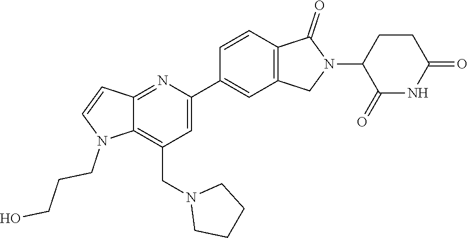 [SMILES: O=C1CCC(N2Cc3cc(-c4cc(CN5CCCC5)c5c(ccn5CCCO)n4)ccc3C2=O)C(=O)N1] | 3-(5-(1-(3-hydroxypropyl)-7- (pyrrolidin-1-ylmethyl)-1H- pyrrolo[3,2-b]pyridin-5-yl)-1- oxoisoindolin-2-yl)piperidine-2,6- dione |
| 174 | 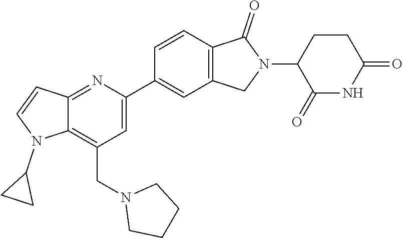 [SMILES: O=C1CCC(N2Cc3cc(-c4cc(CN5CCCC5)c5c(ccn5C5CC5)n4)ccc3C2=O)C(=O)N1] | 3-(5-(1-cyclopropyl-7-(pyrrolidin- 1-ylmethyl)-1H-pyrrolo[3,2- b]pyridin-5-yl)-1-oxoisoindolin-2- yl)piperidine-2,6-dione |
| 175 | 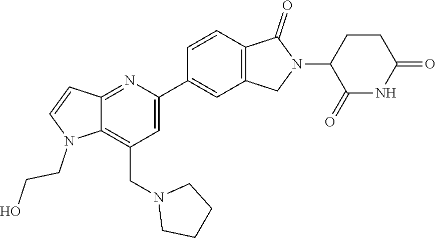 [SMILES: O=C1CCC(N2Cc3cc(-c4cc(CN5CCCC5)c5c(ccn5CCO)n4)ccc3C2=O)C(=O)N1] | 3-(5-(1-(2-hydroxyethyl)-7- (pyrrolidin-1-ylmethyl)-1H- pyrrolo[3,2-b]pyridin-5-yl)-1- oxoisoindolin-2-yl)piperidine-2,6- dione |
| 176 | 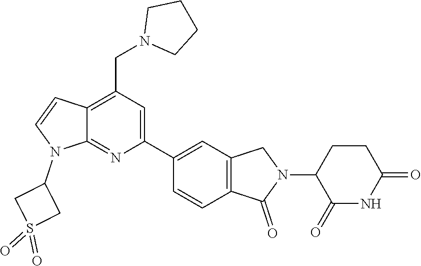 [SMILES: O=C1CCC(N2Cc3cc(-c4cc(CN5CCCC5)c5ccn(C6CS(=O)(=O)C6)c5n4)ccc3C2=O)C(=O)N1] | 3-(5-(1-(1,1-dioxidothietan-3-yl)- 4-(pyrrolidin-1-ylmethyl)-1H- pyrrolo[2,3-b]pyridin-6-yl)-1- oxoisoindolin-2-yl)piperidine-2,6- dione |
| 177 | 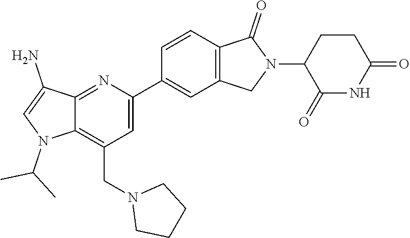 [SMILES: CC(C)n1cc(N)c2nc(-c3ccc4c(c3)CN(C3CCC(=O)NC3=O)C4=O)cc(CN3CCCC3)c21] | 3-(5-(3-amino-1-isopropyl-7- (pyrrolidin-1-ylmethyl)-1H- pyrazolo[4,3-b]pyridin-5-yl)-1- oxoisoindolin-2-yl)piperidine-2,6- dione |
| 178 | 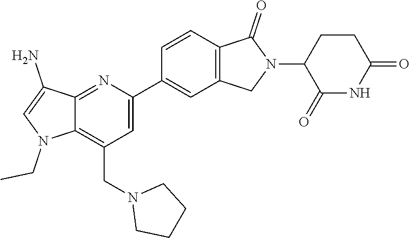 [SMILES: CCn1cc(N)c2nc(-c3ccc4c(c3)CN(C3CCC(=O)NC3=O)C4=O)cc(CN3CCCC3)c21] | 3-(5-(3-amino-1-ethyl-7- (pyrrolidin-1-ylmethyl)-1H- pyrazolo[4,3-b]pyridin-5-yl)-1- oxoisoindolin-2-yl)piperidine-2,6- dione |
| 179 | 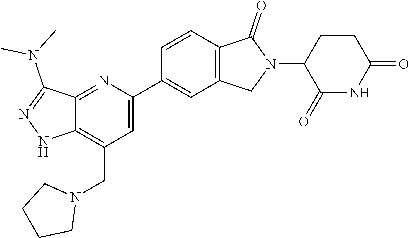 [SMILES: CN(C)c1n[nH]c2c(CN3CCCC3)cc(-c3ccc4c(c3)CN(C3CCC(=O)NC3=O)C4=O)nc12] | 3-(5-(3-(dimethylamino)-7- (pyrrolidin-1-ylmethyl)-1H- pyrazolo[4,3-b]pyridin-5-yl)-1- oxoisoindolin-2-yl)piperidine-2,6- dione |
| 180 | 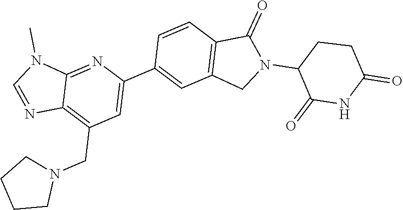 [SMILES: Cn1cnc2c(CN3CCCC3)cc(-c3ccc4c(c3)CN(C3CCC(=O)NC3=O)C4=O)nc21] | 3-(5-(3-methyl-7-(pyrrolidin-1- ylmethyl)-3H-imidazo[4,5- b]pyridin-5-yl)-1-oxoisoindolin-2- yl)piperidine-2,6-dione |
| 181 | 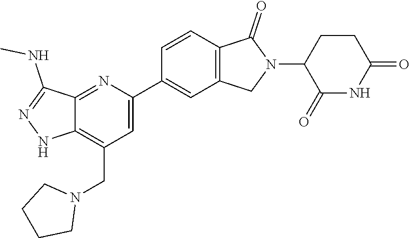 [SMILES: CNc1n[nH]c2c(CN3CCCC3)cc(-c3ccc4c(c3)CN(C3CCC(=O)NC3=O)C4=O)nc12] | 3-(5-(3-(methylamino)-7- (pyrrolidin-1-ylmethyl)-1H- pyrazolo[4,3-b]pyridin-5-yl)-1- oxoisoindolin-2-yl)piperidine-2,6- dione |
| 182 | 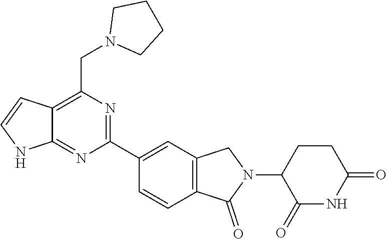 [SMILES: O=C1CCC(N2Cc3cc(-c4nc(CN5CCCC5)c5cc[nH]c5n4)ccc3C2=O)C(=O)N1] | 3-(1-oxo-5-(4-(pyrrolidin-1- ylmethyl)-7H-pyrrolo[2,3- d]pyrimidin-2-yl)isoindolin-2- yl)piperidine-2,6-dione |
| 183 | 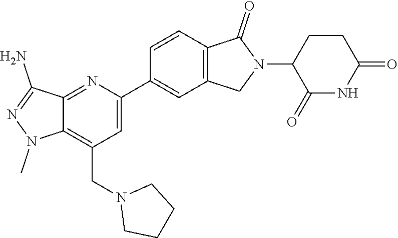 [SMILES: Cn1nc(N)c2nc(-c3ccc4c(c3)CN(C3CCC(=O)NC3=O)C4=O)cc(CN3CCCC3)c21] | 3-(5-(3-amino-1-methyl-7- (pyrrolidin-1-ylmethyl)-1H- pyrazolo[4,3-b]pyridin-5-y1)-1- oxoisoindolin-2-yl)piperidine-2,6- dione |
| 184 | 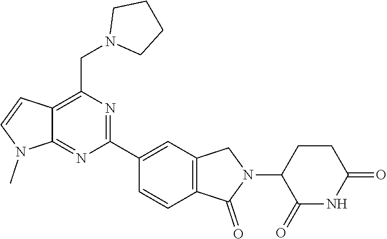 [SMILES: Cn1ccc2c(CN3CCCC3)nc(-c3ccc4c(c3)CN(C3CCC(=O)NC3=O)C4=O)nc21] | 3-(5-(7-methyl-4-(pyrrolidin-1- ylmethyl)-7H-pyrrolo[2,3- d]pyrimidin-2-yl)-1-oxoisoindolin- 2-yl)piperidine-2,6-dione |
| 185 | 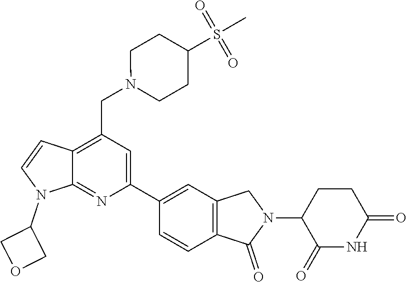 [SMILES: CS(=O)(=O)C1CCN(Cc2cc(-c3ccc4c(c3)CN(C3CCC(=O)NC3=O)C4=O)nc3c2ccn3C2COC2)CC1] | 3-(5-(4-((4- (methylsulfonyl)piperidin-1- yl)methyl)-1-(oxetan-3-yl)-1H- pyrrolo[2,3-b]pyridin-6-yl)-1- oxoisoindolin-2-yl)piperidine-2,6- dione |
| 186 | 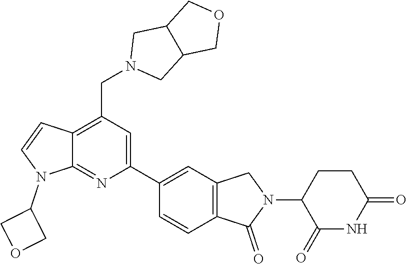 [SMILES: O=C1CCC(N2Cc3cc(-c4cc(CN5CC6COCC6C5)c5ccn(C6COC6)c5n4)ccc3C2=O)C(=O)N1] | 3-(5-(1-(oxetan-3-yl)-4- ((tetrahydro-1H-furo[3,4-c]pyrrol- 5(3H)-yl)methyl)-1H-pyrrolo[2,3- b]pyridin-6-yl)-1-oxoisoindolin-2- yl)piperidine-2,6-dione |
| 187 | 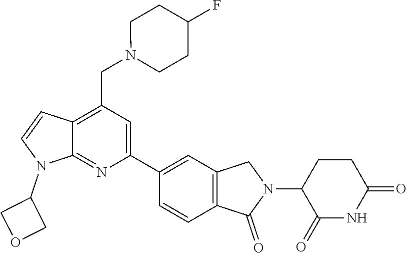 [SMILES: O=C1CCC(N2Cc3cc(-c4cc(CN5CCC(F)CC5)c5ccn(C6COC6)c5n4)ccc3C2=O)C(=O)N1] | 3-(5-(4-((4-fluoropiperidin-1- yl)methyl)-1-(oxetan-3-yl)-1H- pyrrolo[2,3-b]pyridin-6-yl)-1- oxoisoindolin-2-yl)piperidine-2,6- dione |
| 188 | 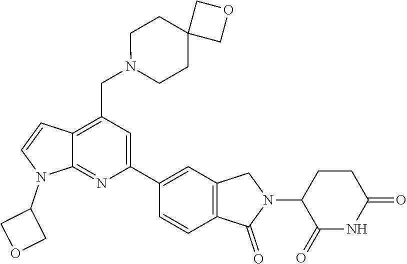 [SMILES: O=C1CCC(N2Cc3cc(-c4cc(CN5CCC6(CC5)COC6)c5ccn(C6COC6)c5n4)ccc3C2=O)C(=O)N1] | 3-(5-(4-((2-oxa-7- azaspiro[3.5]nonan-7-yl)methyl)-1- (oxetan-3-yl)-1H-pyrrolo[2,3- b]pyridin-6-yl)-1-oxoisoindolin-2- yl)piperidine-2,6-dione |
| 189 | 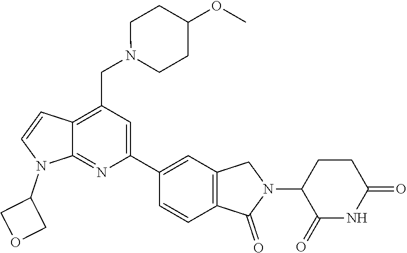 [SMILES: COC1CCN(Cc2cc(-c3ccc4c(c3)CN(C3CCC(=O)NC3=O)C4=O)nc3c2ccn3C2COC2)CC1] | 3-(5-(4-((4-methoxypiperidin-1- yl)methyl)-1-(oxetan-3-yl)-1H- pyrrolo[2,3-b]pyridin-6-yl)-1- oxoisoindolin-2-yl)piperidine-2,6- dione |
| 190 | 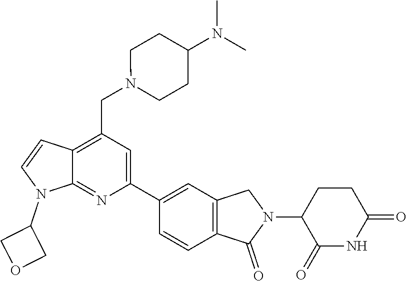 [SMILES: CN(C)C1CCN(Cc2cc(-c3ccc4c(c3)CN(C3CCC(=O)NC3=O)C4=O)nc3c2ccn3C2COC2)CC1] | 3-(5-(4-((4- (dimethylamino)piperidin-1- yl)methyl)-1-(oxetan-3-yl)-1H- pyrrolo[2,3-b]pyridin-6-yl)-1- oxoisoindolin-2-yl)piperidine-2,6- dione |
| 191 | 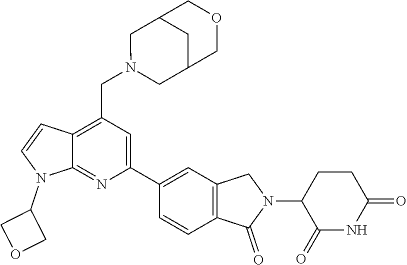 [SMILES: O=C1CCC(N2Cc3cc(-c4cc(CN5CC6COCC(C6)C5)c5ccn(C6COC6)c5n4)ccc3C2=O)C(=O)N1] | 3-(5-(4-((3-oxa-7- azabicyclo[3.3.1]nonan-7- yl)methyl)-1-(oxetan-3-yl)-1H- pyrrolo[2,3-b]pyridin-6-yl)-1- oxoisoindolin-2-yl)piperidine-2,6- dione |
| 192 | 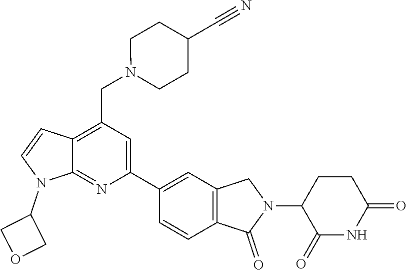 [SMILES: N#CC1CCN(Cc2cc(-c3ccc4c(c3)CN(C3CCC(=O)NC3=O)C4=O)nc3c2ccn3C2COC2)CC1] | 1-((6-(2-(2,6-dioxopiperidin-3-y1)- 1-oxoisoindolin-5-yl)-1-(oxetan-3- yl)-1H-pyrrolo[2,3-b]pyridin-4- yl)methyl)piperidine-4-carbonitrile |
| 193 | 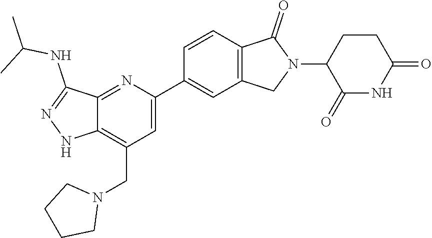 [SMILES: CC(C)Nc1n[nH]c2c(CN3CCCC3)cc(-c3ccc4c(c3)CN(C3CCC(=O)NC3=O)C4=O)nc12] | 3-(5-(3-(isopropylamino)-7- (pyrrolidin-1-ylmethyl)-1H- pyrazolo[4,3-b]pyridin-5-yl)-1- oxoisoindolin-2-yl)piperidine-2,6- dione |
| 194 | 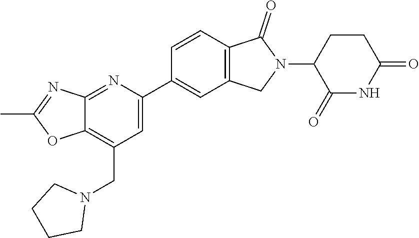 [SMILES: Cc1nc2nc(-c3ccc4c(c3)CN(C3CCC(=O)NC3=O)C4=O)cc(CN3CCCC3)c2o1] | 3-(5-(2-methyl-7-(pyrrolidin-1- ylmethyl)oxazolo[4,5-b]pyridin-5- yl)-1-oxoisoindolin-2- yl)piperidine-2,6-dione |
| 195 | 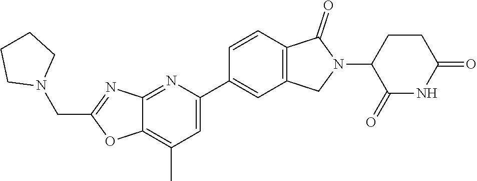 [SMILES: Cc1cc(-c2ccc3c(c2)CN(C2CCC(=O)NC2=O)C3=O)nc2nc(CN3CCCC3)oc12] | 3-(5-(7-methyl-2-(pyrrolidin-1- ylmethyl)oxazolo[4,5-b]pyridin-5- yl)-1-oxoisoindolin-2- yl)piperidine-2,6-dione |
| 196 | 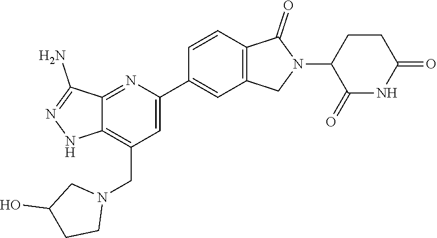 [SMILES: Nc1n[nH]c2c(CN3CCC(O)C3)cc(-c3ccc4c(c3)CN(C3CCC(=O)NC3=O)C4=O)nc12] | 3-(5-(3-amino-7-((3- hydroxypyrrolidin-1-yl)methyl)- 1H-pyrazolo[4,3-b]pyridin-5-yl)-1- oxoisoindolin-2-yl)piperidine-2,6- dione |
| 197 | 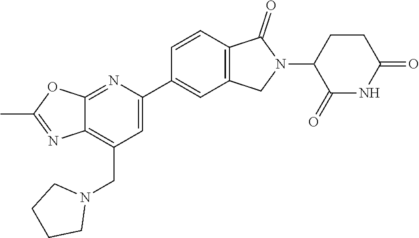 [SMILES: Cc1nc2c(CN3CCCC3)cc(-c3ccc4c(c3)CN(C3CCC(=O)NC3=O)C4=O)nc2o1] | 3-(5-(2-methyl-7-(pyrrolidin-1- ylmethyl)oxazolo[5,4-b]pyridin-5- yl)-1-oxoisoindolin-2- yl)piperidine-2,6-dione |
| 198 | 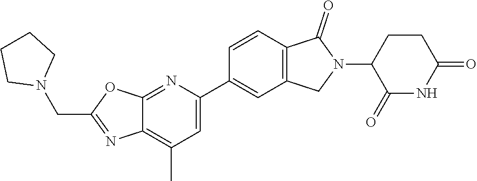 [SMILES: Cc1cc(-c2ccc3c(c2)CN(C2CCC(=O)NC2=O)C3=O)nc2oc(CN3CCCC3)nc12] | 3-(5-(7-methyl-2-(pyrrolidin-1- ylmethyl)oxazolo[5,4-b]pyridin-5- yl)-1-oxoisoindolin-2- yl)piperidine-2,6-dione |
| 199 | 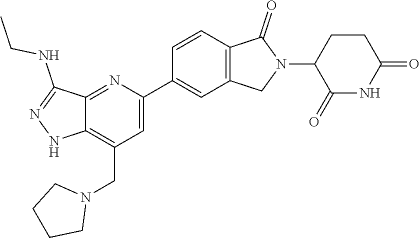 [SMILES: CCNc1n[nH]c2c(CN3CCCC3)cc(-c3ccc4c(c3)CN(C3CCC(=O)NC3=O)C4=O)nc12] | 3-(5-(3-(ethylamino)-7-(pyrrolidin- 1-ylmethyl)-1H-pyrazolo[4,3- b]pyridin-5-yl)-1-oxoisoindolin-2- yl)piperidine-2,6-dione |
| 200 | 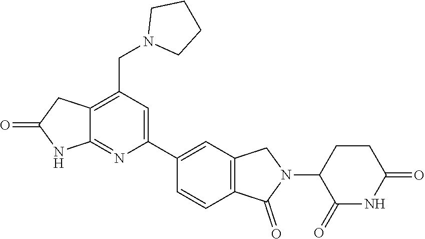 [SMILES: O=C1CCC(N2Cc3cc(-c4cc(CN5CCCC5)c5c(n4)NC(=O)C5)ccc3C2=O)C(=O)N1] | 3-(1-oxo-5-(2-oxo-4-(pyrrolidin-1- ylmethyl)-2,3-dihydro-1H- pyrrolo[2,3-b]pyridin-6- yl)isoindolin-2-yl)piperidine-2,6- dione |
| 201 | 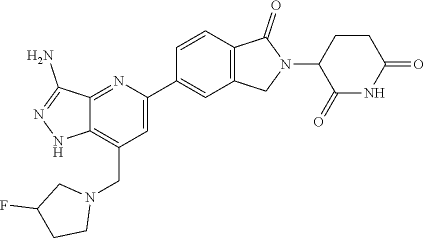 [SMILES: Nc1n[nH]c2c(CN3CCC(F)C3)cc(-c3ccc4c(c3)CN(C3CCC(=O)NC3=O)C4=O)nc12] | 3-(5-(3-amino-7-((3- fluoropyrrolidin-1-yl)methyl)-1H- pyrazolo[4,3-b]pyridin-5-yl)-1- oxoisoindolin-2-yl)piperidine-2,6- dione |
| 202 | 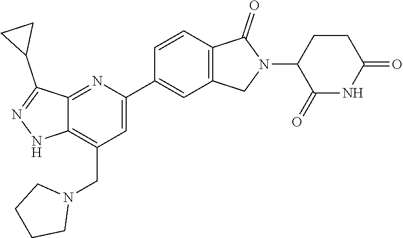 [SMILES: O=C1CCC(N2Cc3cc(-c4cc(CN5CCCC5)c5[nH]nc(C6CC6)c5n4)ccc3C2=O)C(=O)N1] | 3-(5-(3-cyclopropyl-7-(pyrrolidin- 1-ylmethyl)-1H-pyrazolo[4,3- b]pyridin-5-yl)-1-oxoisoindolin-2- yl)piperidine-2,6-dione |
| 203 | 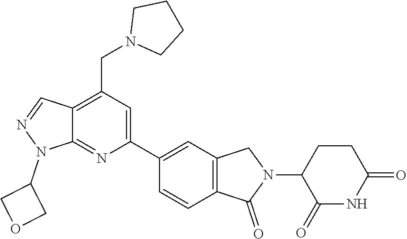 [SMILES: O=C1CCC(N2Cc3cc(-c4cc(CN5CCCC5)c5cnn(C6COC6)c5n4)ccc3C2=O)C(=O)N1] | 3-(5-(1-(oxetan-3-y1)-4-(pyrrolidin- 1-ylmethyl)-1H-pyrazolo[3,4- b]pyridin-6-yl)-1-oxoisoindolin-2- yl)piperidine-2,6-dione |
| 204 | 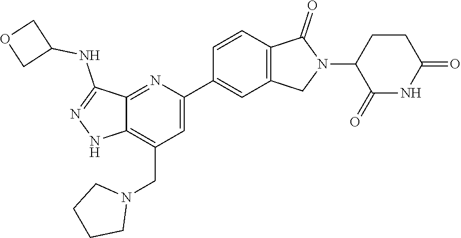 [SMILES: O=C1CCC(N2Cc3cc(-c4cc(CN5CCCC5)c5[nH]nc(NC6COC6)c5n4)ccc3C2=O)C(=O)N1] | 3-(5-(3-(oxetan-3-ylamino)-7- (pyrrolidin-1-ylmethyl)-1H- pyrazolo[4,3-b]pyridin-5-yl)-1- oxoisoindolin-2-yl)piperidine-2,6- dione |
| 205 | 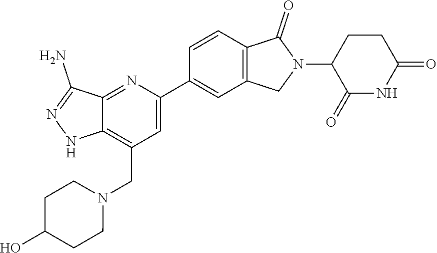 [SMILES: Nc1n[nH]c2c(CN3CCC(O)CC3)cc(-c3ccc4c(c3)CN(C3CCC(=O)NC3=O)C4=O)nc12] | 3-(5-(3-amino-7-((4- hydroxypiperidin-1-yl)methyl)-1H- pyrazolo[4,3-b]pyridin-5-yl)-1- oxoisoindolin-2-yl)piperidine-2,6- dione |
| 206 | 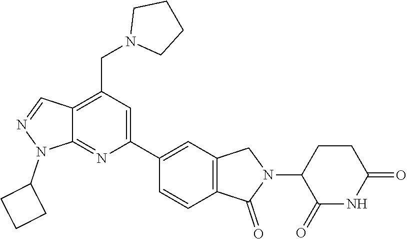 [SMILES: O=C1CCC(N2Cc3cc(-c4cc(CN5CCCC5)c5cnn(C6CCC6)c5n4)ccc3C2=O)C(=O)N1] | 3-(5-(1-cyclobutyl-4-(pyrrolidin-1- ylmethyl)-1H-pyrazolo[3,4- b]pyridin-6-yl)-1-oxoisoindolin-2- yl)piperidine-2,6-dione |
| 207 | 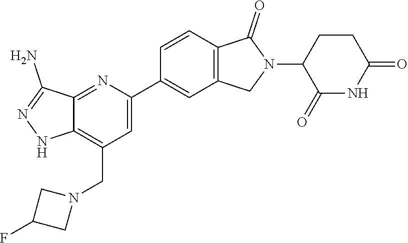 [SMILES: Nc1n[nH]c2c(CN3CC(F)C3)cc(-c3ccc4c(c3)CN(C3CCC(=O)NC3=O)C4=O)nc12] | 3-(5-(3-amino-7-((3-fluoroazetidin- 1-yl)methyl)-1H-pyrazolo[4,3- b]pyridin-5-yl)-1-oxoisoindolin-2- yl)piperidine-2,6-dione |
| 208 | 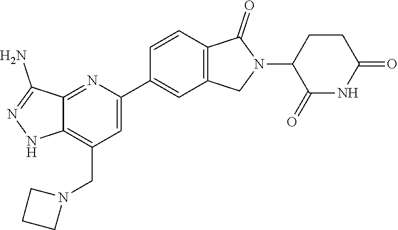 [SMILES: Nc1n[nH]c2c(CN3CCC3)cc(-c3ccc4c(c3)CN(C3CCC(=O)NC3=O)C4=O)nc12] | 3-(5-(3-amino-7-(azetidin-1- ylmethyl)-1H-pyrazolo[4,3- b]pyridin-5-yl)-1-oxoisoindolin-2- yl)piperidine-2,6-dione |
| 209 | 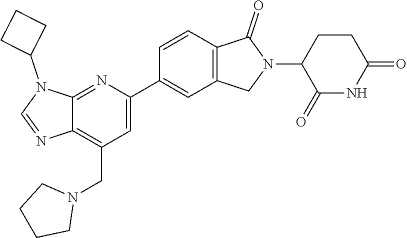 [SMILES: O=C1CCC(N2Cc3cc(-c4cc(CN5CCCC5)c5ncn(C6CCC6)c5n4)ccc3C2=O)C(=O)N1] | 3-(5-(3-cyclobutyl-7-(pyrrolidin-1- ylmethyl)-3H-imidazo[4,5- b]pyridin-5-yl)-1-oxoisoindolin-2- yl)piperidine-2,6-dione |
| 210 | 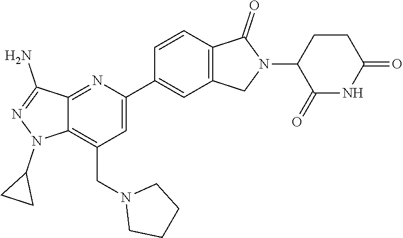 [SMILES: Nc1nn(C2CC2)c2c(CN3CCCC3)cc(-c3ccc4c(c3)CN(C3CCC(=O)NC3=O)C4=O)nc12] | 3-(5-(3-amino-1-cyclopropyl-7- (pyrrolidin-1-ylmethyl)-1H- pyrazolo[4,3-b]pyridin-5-yl)-1- oxoisoindolin-2-yl)piperidine-2,6- dione |
| 211 | 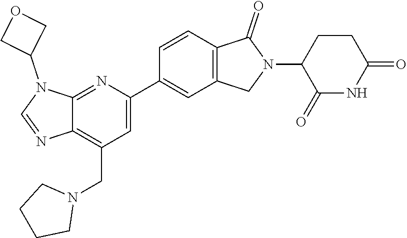 [SMILES: O=C1CCC(N2Cc3cc(-c4cc(CN5CCCC5)c5ncn(C6COC6)c5n4)ccc3C2=O)C(=O)N1] | 3-(5-(3-(oxetan-3-yl)-7-(pyrrolidin- 1-ylmethyl)-3H-imidazo[4,5- b]pyridin-5-yl)-1-oxoisoindolin-2- yl)piperidine-2,6-dione |
| 212 | 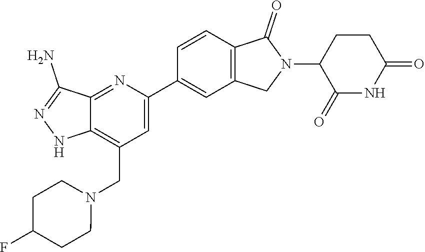 [SMILES: Nc1n[nH]c2c(CN3CCC(F)CC3)cc(-c3ccc4c(c3)CN(C3CCC(=O)NC3=O)C4=O)nc12] | 3-(5-(3-amino-7-((4- fluoropiperidin-1-yl)methyl)-1H- pyrazolo[4,3-b]pyridin-5-yl)-1- oxoisoindolin-2-yl)piperidine-2,6- dione |
| 213 | 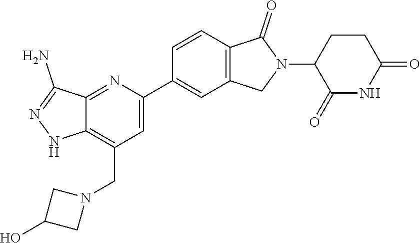 [SMILES: Nc1n[nH]c2c(CN3CC(O)C3)cc(-c3ccc4c(c3)CN(C3CCC(=O)NC3=O)C4=O)nc12] | 3-(5-(3-amino-7-((3- hydroxyazetidin-1-yl)methyl)-1H- pyrazolo[4,3-b]pyridin-5-yl)-1- oxoisoindolin-2-yl)piperidine-2,6- dione |
| 214 | 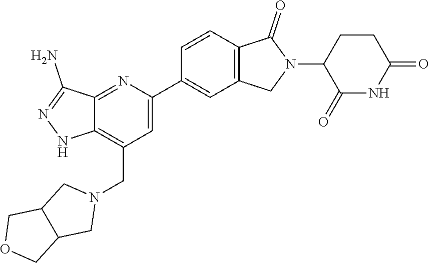 [SMILES: Nc1n[nH]c2c(CN3CC4COCC4C3)cc(-c3ccc4c(c3)CN(C3CCC(=O)NC3=O)C4=O)nc12] | 3-(5-(3-amino-7-((tetrahydro-1H- furo[3,4-c]pyrrol-5(3H)- yl)methyl)-1H-pyrazolo[4,3- b]pyridin-5-yl)-1-oxoisoindolin-2- yl)piperidine-2,6-dione |
| 215 | 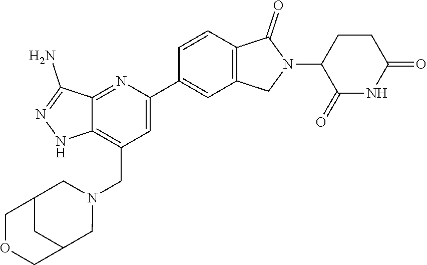 [SMILES: Nc1n[nH]c2c(CN3CC4COCC(C4)C3)cc(-c3ccc4c(c3)CN(C3CCC(=O)NC3=O)C4=O)nc12] | 3-(5-(7-((3-oxa-7- azabicyclo[3.3.1]nonan-7- yl)methyl)-3-amino-1H- pyrazolo[4,3-b]pyridin-5-yl)-1- oxoisoindolin-2-yl)piperidine-2,6- dione |
| 216 | 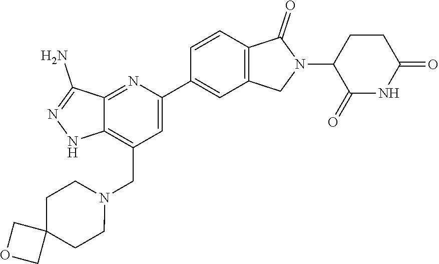 [SMILES: Nc1n[nH]c2c(CN3CCC4(CC3)COC4)cc(-c3ccc4c(c3)CN(C3CCC(=O)NC3=O)C4=O)nc12] | 3-(5-(7-((2-oxa-7- azaspiro[3.5]nonan-7-yl)methyl)-3- amino-1H-pyrazolo[4,3-b]pyridin- 5-yl)-1-oxoisoindolin-2- yl)piperidine-2,6-dione |
| 217 | 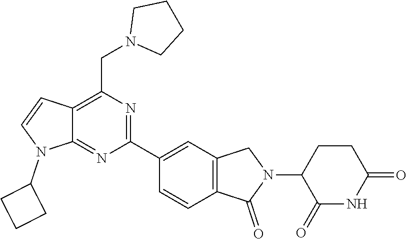 [SMILES: O=C1CCC(N2Cc3cc(-c4nc(CN5CCCC5)c5ccn(C6CCC6)c5n4)ccc3C2=O)C(=O)N1] | 3-(5-(7-cyclobutyl-4-(pyrrolidin-1- ylmethyl)-7H-pyrrolo[2,3- d]pyrimidin-2-yl)-1-oxoisoindolin- 2-yl)piperidine-2,6-dione |
| 218 | 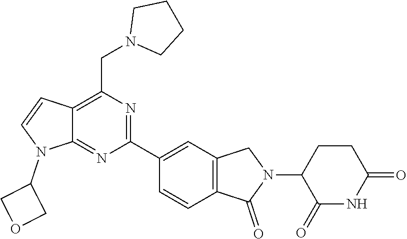 [SMILES: O=C1CCC(N2Cc3cc(-c4nc(CN5CCCC5)c5ccn(C6COC6)c5n4)ccc3C2=O)C(=O)N1] | 3-(5-(7-(oxetan-3-yl)-4-(pyrrolidin- 1-ylmethyl)-7H-pyrrolo[2,3- d]pyrimidin-2-yl)-1-oxoisoindolin- 2-yl)piperidine-2,6-dione |
| 219 | 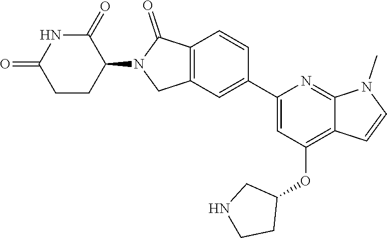 [SMILES: Cn1ccc2c(O[C@@H]3CCNC3)cc(-c3ccc4c(c3)CN([C@H]3CCC(=O)NC3=O)C4=O)nc21] | (S)-3-(5-(1-methyl-4-(((R)- pyrrolidin-3-yl)oxy)-1H- pyrrolo[2,3-b]pyridin-6-yl)-1- oxoisoindolin-2-yl)piperidine-2,6- dione |
| 220 | 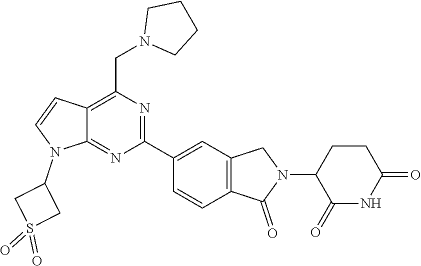 [SMILES: O=C1CCC(N2Cc3cc(-c4nc(CN5CCCC5)c5ccn(C6CS(=O)(=O)C6)c5n4)ccc3C2=O)C(=O)N1] | 3-(5-(7-(1,1-dioxidothietan-3-yl)- 4-(pyrrolidin-1-ylmethyl)-7H- pyrrolo[2,3-d]pyrimidin-2-yl)-1- oxoisoindolin-2-yl)piperidine-2,6- dione |
| 221 | 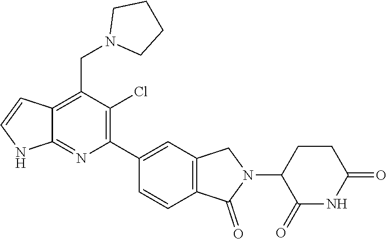 [SMILES: O=C1CCC(N2Cc3cc(-c4nc5[nH]ccc5c(CN5CCCC5)c4Cl)ccc3C2=O)C(=O)N1] | 3-(5-(5-chloro-4-(pyrrolidin-1- ylmethyl)-1H-pyrrolo[2,3- b]pyridin-6-yl)-1-oxoisoindolin-2- yl)piperidine-2,6-dione |
| 222 | 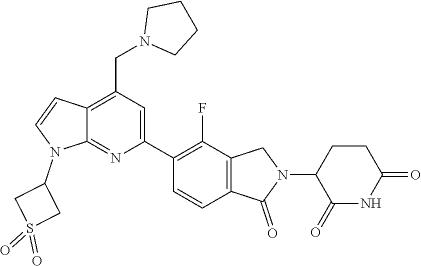 [SMILES: O=C1CCC(N2Cc3c(ccc(-c4cc(CN5CCCC5)c5ccn(C6CS(=O)(=O)C6)c5n4)c3F)C2=O)C(=O)N1] | 3-(5-(1-(1,1-dioxidothietan-3-yl)- 4-(pyrrolidin-1-ylmethyl)-1H- pyrrolo[2,3-b]pyridin-6-yl)-4- fluoro-1-oxoisoindolin-2- yl)piperidine-2,6-dione |
| 223 | 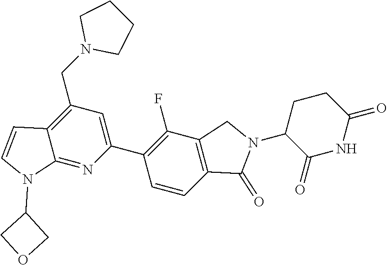 [SMILES: O=C1CCC(N2Cc3c(ccc(-c4cc(CN5CCCC5)c5ccn(C6COC6)c5n4)c3F)C2=O)C(=O)N1] | 3-(4-fluoro-5-(1-(oxetan-3-yl)-4- (pyrrolidin-1-ylmethyl)-1H- pyrrolo[2,3-b]pyridin-6-yl)-1- oxoisoindolin-2-yl)piperidine-2,6- dione |
| 224 | 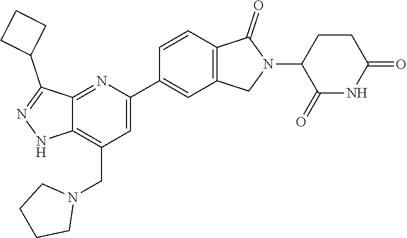 [SMILES: O=C1CCC(N2Cc3cc(-c4cc(CN5CCCC5)c5[nH]nc(C6CCC6)c5n4)ccc3C2=O)C(=O)N1] | 3-(5-(3-cyclobutyl-7-(pyrrolidin-1- ylmethyl)-1H-pyrazolo[4,3- b]pyridin-5-yl)-1-oxoisoindolin-2- yl)piperidine-2,6-dione |
| 225 | 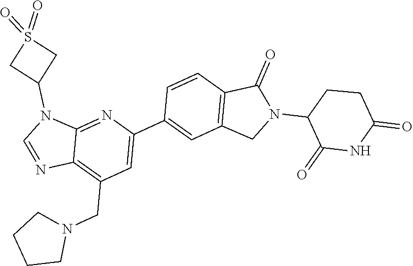 [SMILES: O=C1CCC(N2Cc3cc(-c4cc(CN5CCCC5)c5ncn(C6CS(=O)(=O)C6)c5n4)ccc3C2=O)C(=O)N1] | 3-(5-(3-(1,1-dioxidothietan-3-yl)- 7-(pyrrolidin-1-ylmethyl)-3H- imidazo[4,5-b]pyridin-5-yl)-1- oxoisoindolin-2-yl)piperidine-2,6- dione |
| 226 | 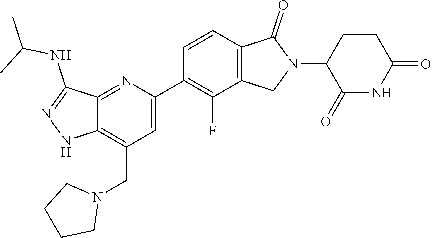 [SMILES: CC(C)Nc1n[nH]c2c(CN3CCCC3)cc(-c3ccc4c(c3F)CN(C3CCC(=O)NC3=O)C4=O)nc12] | 3-(4-fluoro-5-(3-(isopropylamino)- 7-(pyrrolidin-1-ylmethyl)-1H- pyrazolo[4,3-b]pyridin-5-yl)-1- oxoisoindolin-2-yl)piperidine-2,6- dione |
| 227 | 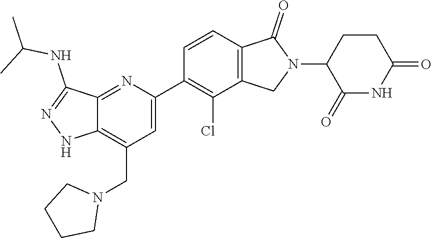 [SMILES: CC(C)Nc1n[nH]c2c(CN3CCCC3)cc(-c3ccc4c(c3Cl)CN(C3CCC(=O)NC3=O)C4=O)nc12] | 3-(4-chloro-5-(3-(isopropylamino)- 7-(pyrrolidin-1-ylmethyl)-1H- pyrazolo[4,3-b]pyridin-5-yl)-1- oxoisoindolin-2-yl)piperidine-2,6- dione |
| 228 | 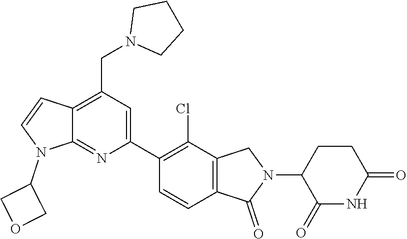 [SMILES: O=C1CCC(N2Cc3c(ccc(-c4cc(CN5CCCC5)c5ccn(C6COC6)c5n4)c3Cl)C2=O)C(=O)N1] | 3-(4-chloro-5-(1-(oxetan-3-yl)-4- (pyrrolidin-1-ylmethyl)-1H- pyrrolo[2,3-b]pyridin-6-yl)-1- oxoisoindolin-2-yl)piperidine-2,6- dione |
| 229 | 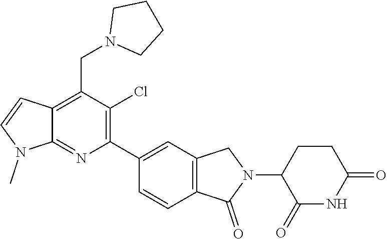 [SMILES: Cn1ccc2c(CN3CCCC3)c(Cl)c(-c3ccc4c(c3)CN(C3CCC(=O)NC3=O)C4=O)nc21] | 3-(5-(5-chloro-1-methyl-4- (pyrrolidin-1-ylmethyl)-1H- pyrrolo[2,3-b]pyridin-6-yl)-1- oxoisoindolin-2-yl)piperidine-2,6- dione |
| 230 | 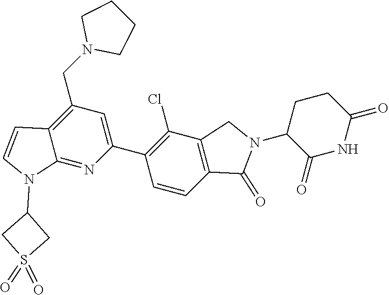 [SMILES: O=C1CCC(N2Cc3c(ccc(-c4cc(CN5CCCC5)c5ccn(C6CS(=O)(=O)C6)c5n4)c3Cl)C2=O)C(=O)N1] | 3-(4-chloro-5-(1-(1,1- dioxidothietan-3-yl)-4-(pyrrolidin- 1-ylmethyl)-1H-pyrrolo[2,3- b]pyridin-6-yl)-1-oxoisoindolin-2- yl)piperidine-2,6-dione |
| 231 | 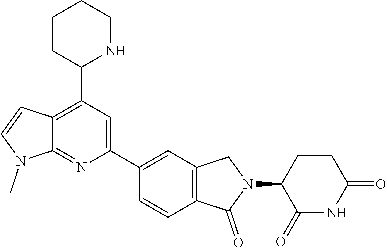 [SMILES: Cn1ccc2c(C3CCCCN3)cc(-c3ccc4c(c3)CN([C@H]3CCC(=O)NC3=O)C4=O)nc21] | (3S)-3-(5-(1-methyl-4-(piperidin-2- yl)-1H-pyrrolo[2,3-b]pyridin-6-yl)- 1-oxoisoindolin-2-yl)piperidine- 2,6-dione |
| 232 | 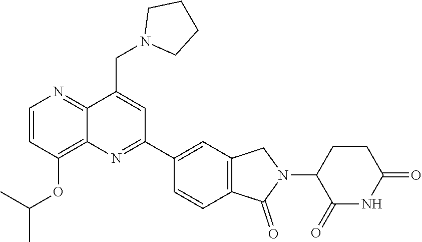 [SMILES: CC(C)Oc1ccnc2c(CN3CCCC3)cc(-c3ccc4c(c3)CN(C3CCC(=O)NC3=O)C4=O)nc12] | 3-(5-(8-isopropoxy-4-(pyrrolidin- 1-ylmethyl)-1,5-naphthyridin-2- yl)-1-oxoisoindolin-2- yl)piperidine-2,6-dione |
| 233 | 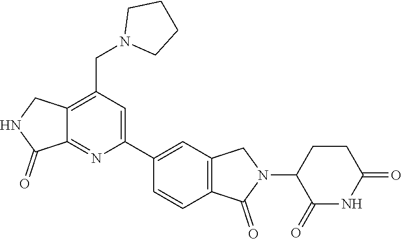 [SMILES: O=C1CCC(N2Cc3cc(-c4cc(CN5CCCC5)c5c(n4)C(=O)NC5)ccc3C2=O)C(=O)N1] | 3-(1-oxo-5-(7-oxo-4-(pyrrolidin-1- ylmethyl)-6,7-dihydro-5H- pyrrolo[3,4-b]pyridin-2- yl)isoindolin-2-yl)piperidine-2,6- dione |
| 234 | 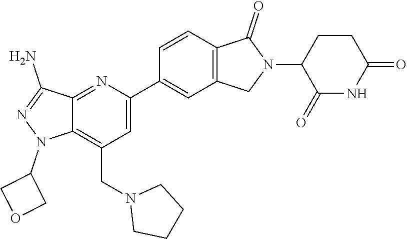 [SMILES: Nc1nn(C2COC2)c2c(CN3CCCC3)cc(-c3ccc4c(c3)CN(C3CCC(=O)NC3=O)C4=O)nc12] | 3-(5-(3-amino-1-(oxetan-3-yl)-7- (pyrrolidin-1-ylmethyl)-1H- pyrazolo[4,3-b]pyridin-5-yl)-1- oxoisoindolin-2-yl)piperidine-2,6- dione |
| 235 | 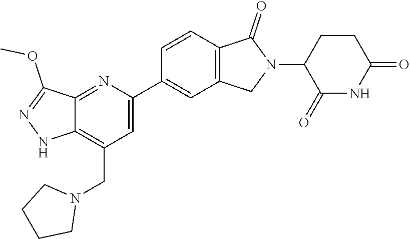 [SMILES: COc1n[nH]c2c(CN3CCCC3)cc(-c3ccc4c(c3)CN(C3CCC(=O)NC3=O)C4=O)nc12] | 3-(5-(3-methoxy-7-(pyrrolidin-1- ylmethyl)-1H-pyrazolo[4,3- b]pyridin-5-yl)-1-oxoisoindolin-2- yl)piperidine-2,6-dione |
| 236 | 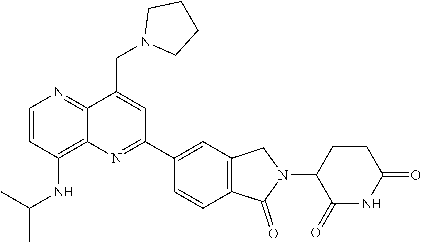 [SMILES: CC(C)Nc1ccnc2c(CN3CCCC3)cc(-c3ccc4c(c3)CN(C3CCC(=O)NC3=O)C4=O)nc12] | 3-(5-(8-(isopropylamino)-4- (pyrrolidin-1-ylmethyl)-1,5- naphthyridin-2-yl)-1- oxoisoindolin-2-yl)piperidine-2,6- dione |
| 237 | 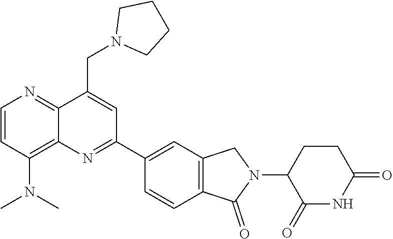 [SMILES: CN(C)c1ccnc2c(CN3CCCC3)cc(-c3ccc4c(c3)CN(C3CCC(=O)NC3=O)C4=O)nc12] | 3-(5-(8-(dimethylamino)-4- (pyrrolidin-1-ylmethyl)-1,5- naphthyridin-2-yl)-1- oxoisoindolin-2-yl)piperidine-2,6- dione |
| 238 | 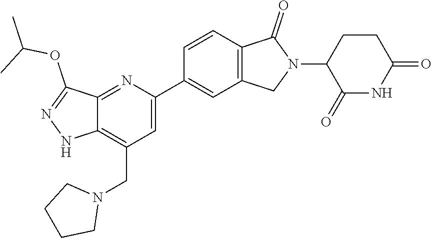 [SMILES: CC(C)Oc1n[nH]c2c(CN3CCCC3)cc(-c3ccc4c(c3)CN(C3CCC(=O)NC3=O)C4=O)nc12] | 3-(5-(3-isopropoxy-7-(pyrrolidin- 1-ylmethyl)-1H-pyrazolo[4,3- b]pyridin-5-yl)-1-oxoisoindolin-2- yl)piperidine-2,6-dione |
| 239 | 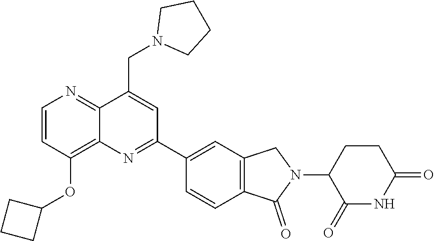 [SMILES: O=C1CCC(N2Cc3cc(-c4cc(CN5CCCC5)c5nccc(OC6CCC6)c5n4)ccc3C2=O)C(=O)N1] | 3-(5-(8-cyclobutoxy-4-(pyrrolidin- 1-ylmethyl)-1,5-naphthyridin-2- yl)-1-oxoisoindolin-2- yl)piperidine-2,6-dione |
| 240 | 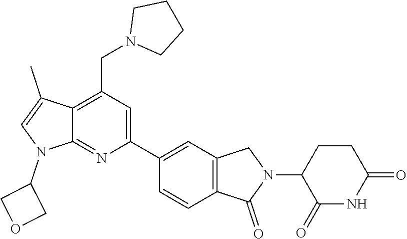 [SMILES: Cc1cn(C2COC2)c2nc(-c3ccc4c(c3)CN(C3CCC(=O)NC3=O)C4=O)cc(CN3CCCC3)c12] | 3-(5-(3-methyl-1-(oxetan-3-yl)-4- (pyrrolidin-1-ylmethyl)-1H- pyrrolo[2,3-b]pyridin-6-yl)-1- oxoisoindolin-2-yl)piperidine-2,6- dione |
| 241 | 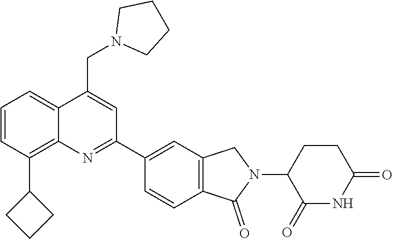 [SMILES: O=C1CCC(N2Cc3cc(-c4cc(CN5CCCC5)c5cccc(C6CCC6)c5n4)ccc3C2=O)C(=O)N1] | 3-(5-(8-cyclobutyl-4-(pyrrolidin-1- ylmethyl)quinolin-2-yl)-1- oxoisoindolin-2-yl)piperidine-2,6- dione |
| 242 | 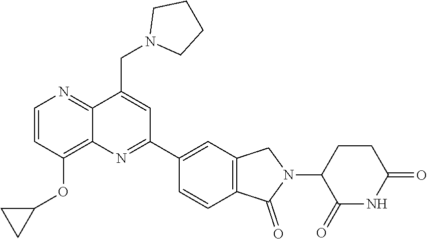 [SMILES: O=C1CCC(N2Cc3cc(-c4cc(CN5CCCC5)c5nccc(OC6CC6)c5n4)ccc3C2=O)C(=O)N1] | 3-(5-(8-cyclopropoxy-4- (pyrrolidin-1-ylmethyl)-1,5- naphthyridin-2-yl)-1- oxoisoindolin-2-yl)piperidine-2,6- dione |
| 243 | 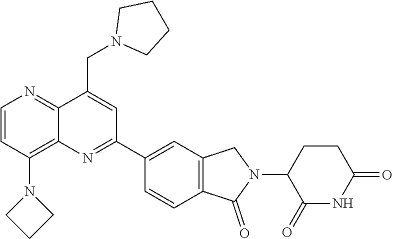 [SMILES: O=C1CCC(N2Cc3cc(-c4cc(CN5CCCC5)c5nccc(N6CCC6)c5n4)ccc3C2=O)C(=O)N1] | 3-(5-(8-(azetidin-1-yl)-4- (pyrrolidin-1-ylmethyl)-1,5- naphthyridin-2-yl)-1- oxoisoindolin-2-yl)piperidine-2,6- dione |
| 244 | 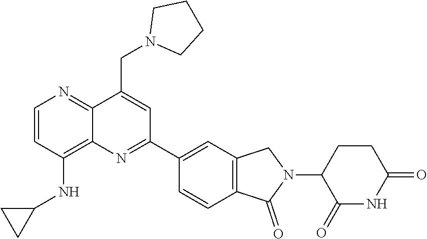 [SMILES: O=C1CCC(N2Cc3cc(-c4cc(CN5CCCC5)c5nccc(NC6CC6)c5n4)ccc3C2=O)C(=O)N1] | 3-(5-(8-(cyclopropylamino)-4- (pyrrolidin-1-ylmethyl)-1,5- naphthyridin-2-yl)-1- oxoisoindolin-2-yl)piperidine-2,6- dione |
| 245 | 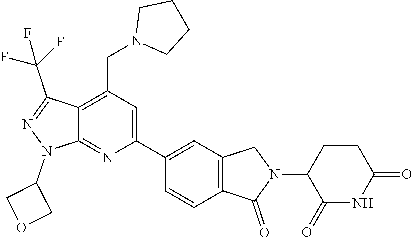 [SMILES: O=C1CCC(N2Cc3cc(-c4cc(CN5CCCC5)c5c(C(F)(F)F)nn(C6COC6)c5n4)ccc3C2=O)C(=O)N1] | 3-(5-(1-(oxetan-3-yl)-4-(pyrrolidin- 1-ylmethyl)-3-(trifluoromethyl)- 1H-pyrazolo[3,4-b]pyridin-6-yl)-1- oxoisoindolin-2-yl)piperidine-2,6- dione |
| 246 | 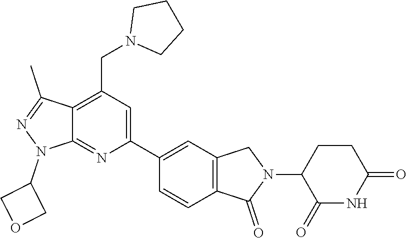 [SMILES: Cc1nn(C2COC2)c2nc(-c3ccc4c(c3)CN(C3CCC(=O)NC3=O)C4=O)cc(CN3CCCC3)c12] | 3-(5-(3-methyl-1-(oxetan-3-yl)-4- (pyrrolidin-1-ylmethyl)-1H- pyrazolo[3,4-b]pyridin-6-yl)-1- oxoisoindolin-2-yl)piperidine-2,6- dione |
| 247 | 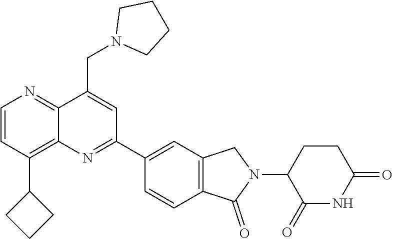 [SMILES: O=C1CCC(N2Cc3cc(-c4cc(CN5CCCC5)c5nccc(C6CCC6)c5n4)ccc3C2=O)C(=O)N1] | 3-(5-(8-cyclobutyl-4-(pyrrolidin-1- ylmethyl)-1,5-naphthyridin-2-yl)- 1-oxoisoindolin-2-yl)piperidine- 2,6-dione |
| 248 | 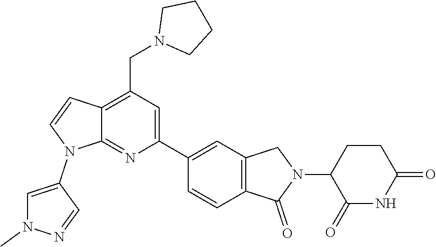 [SMILES: Cn1cc(-n2ccc3c(CN4CCCC4)cc(-c4ccc5c(c4)CN(C4CCC(=O)NC4=O)C5=O)nc32)cn1] | 3-(5-(1-(1-methyl-1H-pyrazol-4- yl)-4-(pyrrolidin-1-ylmethyl)-1H- pyrrolo[2,3-b]pyridin-6-yl)-1- oxoisoindolin-2-yl)piperidine-2,6- dione |
| 249 | 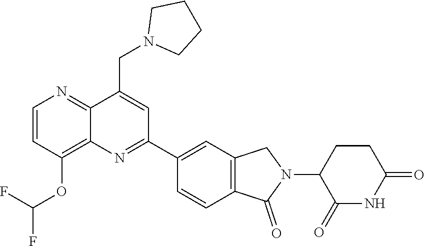 [SMILES: O=C1CCC(N2Cc3cc(-c4cc(CN5CCCC5)c5nccc(OC(F)F)c5n4)ccc3C2=O)C(=O)N1] | 3-(5-(8-(difluoromethoxy)-4- (pyrrolidin-1-ylmethyl)-1,5- naphthyridin-2-yl)-1- oxoisoindolin-2-yl)piperidine-2,6- dione |
| 250 | 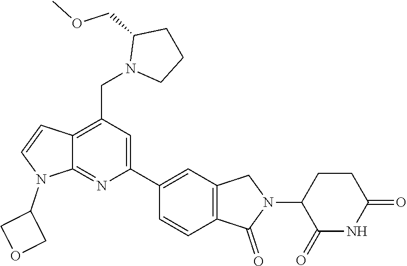 [SMILES: COC[C@@H]1CCCN1Cc1cc(-c2ccc3c(c2)CN(C2CCC(=O)NC2=O)C3=O)nc2c1ccn2C1COC1] | 3-(5-(4-(((S)-2- (methoxymethyl)pyrrolidin-1- yl)methyl)-1-(oxetan-3-yl)-1H- pyrrolo[2,3-b]pyridin-6-yl)-1- oxoisoindolin-2-yl)piperidine-2,6- dione |
| 251 | 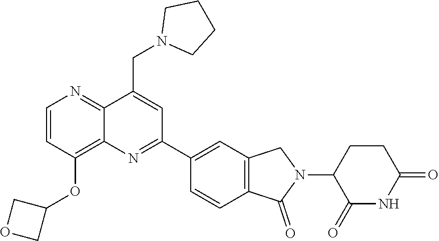 [SMILES: O=C1CCC(N2Cc3cc(-c4cc(CN5CCCC5)c5nccc(OC6COC6)c5n4)ccc3C2=O)C(=O)N1] | 3-(5-(8-(oxetan-3-yloxy)-4- (pyrrolidin-1-ylmethyl)-1,5- naphthyridin-2-yl)-1- oxoisoindolin-2-yl)piperidine-2,6- dione |
| 252 | 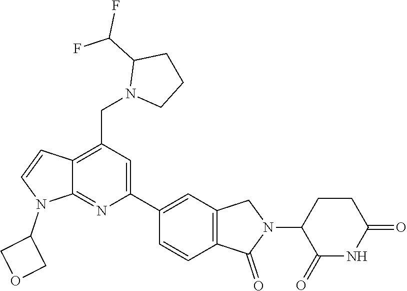 [SMILES: O=C1CCC(N2Cc3cc(-c4cc(CN5CCCC5C(F)F)c5ccn(C6COC6)c5n4)ccc3C2=O)C(=O)N1] | 3-(5-(4-((2- (difluoromethyl)pyrrolidin-1- yl)methyl)-1-(oxetan-3-yl)-1H- pyrrolo[2,3-b]pyridin-6-yl)-1- oxoisoindolin-2-yl)piperidine-2,6- dione |
| 253 | 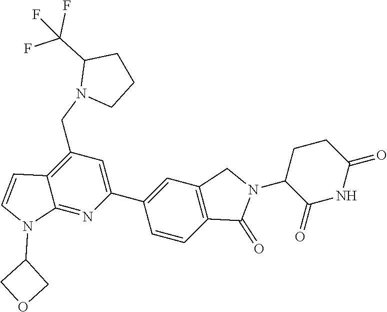 [SMILES: O=C1CCC(N2Cc3cc(-c4cc(CN5CCCC5C(F)(F)F)c5ccn(C6COC6)c5n4)ccc3C2=O)C(=O)N1] | 3-(5-(1-(oxetan-3-yl)-4-((2- (trifluoromethyl)pyrrolidin-1- yl)methyl)-1H-pyrrolo[2,3- b]pyridin-6-yl)-1-oxoisoindolin-2- yl)piperidine-2,6-dione |
| 254 | 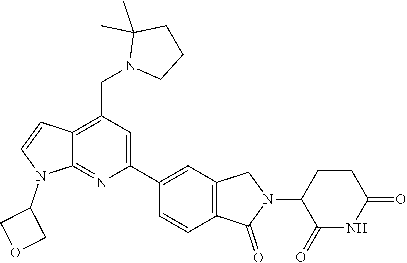 [SMILES: CC1(C)CCCN1Cc1cc(-c2ccc3c(c2)CN(C2CCC(=O)NC2=O)C3=O)nc2c1ccn2C1COC1] | 3-(5-(4-((2,2-dimethylpyrrolidin-1- yl)methyl)-1-(oxetan-3-yl)-1H- pyrrolo[2,3-b]pyridin-6-yl)-1- oxoisoindolin-2-yl)piperidine-2,6- dione |
| 255 | 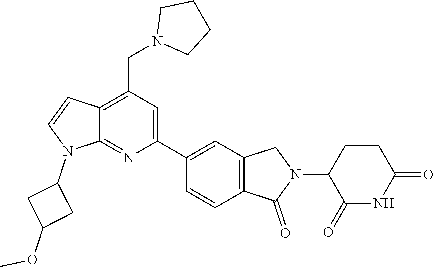 [SMILES: COC1CC(n2ccc3c(CN4CCCC4)cc(-c4ccc5c(c4)CN(C4CCC(=O)NC4=O)C5=O)nc32)C1] | 3-(5-(1-(3-methoxycyclobutyl)-4- (pyrrolidin-1-ylmethyl)-1H- pyrrolo[2,3-b]pyridin-6-yl)-1- oxoisoindolin-2-yl)piperidine-2,6- dione |
| 256 | 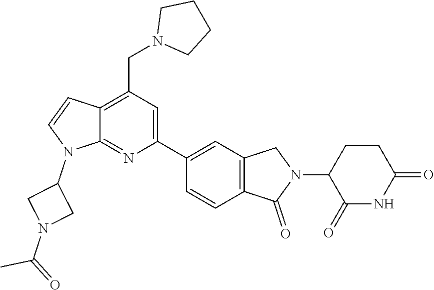 [SMILES: CC(=O)N1CC(n2ccc3c(CN4CCCC4)cc(-c4ccc5c(c4)CN(C4CCC(=O)NC4=O)C5=O)nc32)C1] | 3-(5-(1-(1-acetylazetidin-3-yl)-4- (pyrrolidin-1-ylmethyl)-1H- pyrrolo[2,3-b]pyridin-6-yl)-1- oxoisoindolin-2-yl)piperidine-2,6- dione |
| 257 | 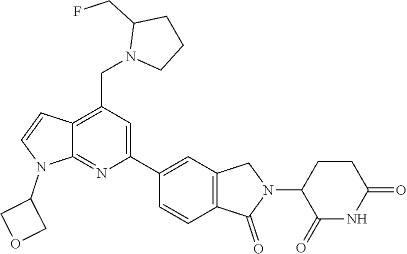 [SMILES: O=C1CCC(N2Cc3cc(-c4cc(CN5CCCC5CF)c5ccn(C6COC6)c5n4)ccc3C2=O)C(=O)N1] | 3-(5-(4-((2- (fluoromethyl)pyrrolidin-1- yl)methyl)-1-(oxetan-3-yl)-1H- pyrrolo[2,3-b]pyridin-6-yl)-1- oxoisoindolin-2-yl)piperidine-2,6- dione |
| 258 | 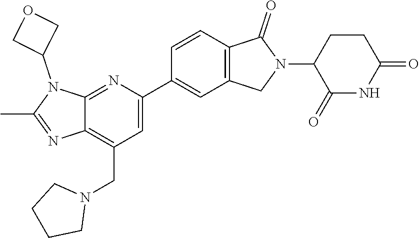 [SMILES: Cc1nc2c(CN3CCCC3)cc(-c3ccc4c(c3)CN(C3CCC(=O)NC3=O)C4=O)nc2n1C1COC1] | 3-(5-(2-methyl-3-(oxetan-3-yl)-7- (pyrrolidin-1-ylmethyl)-3H- imidazo[4,5-b]pyridin-5-yl)-1- oxoisoindolin-2-yl)piperidine-2,6- dione |
| 259 | 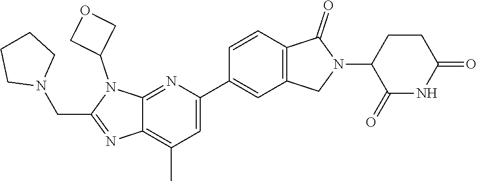 [SMILES: Cc1cc(-c2ccc3c(c2)CN(C2CCC(=O)NC2=O)C3=O)nc2c1nc(CN1CCCC1)n2C1COC1] | 3-(5-(7-methyl-3-(oxetan-3-yl)-2- (pyrrolidin-1-ylmethyl)-3H- imidazo[4,5-b]pyridin-5-yl)-1- oxoisoindolin-2-yl)piperidine-2,6- dione |
| 260 | 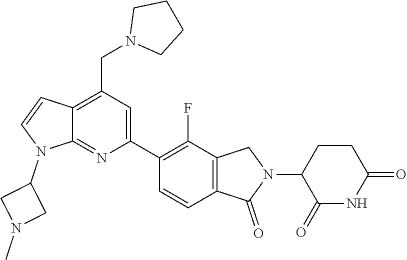 [SMILES: CN1CC(n2ccc3c(CN4CCCC4)cc(-c4ccc5c(c4F)CN(C4CCC(=O)NC4=O)C5=O)nc32)C1] | 3-(4-fluoro-5-(1-(1-methylazetidin- 3-yl)-4-(pyrrolidin-1-ylmethyl)- 1H-pyrrolo[2,3-b]pyridin-6-yl)-1- oxoisoindolin-2-yl)piperidine-2,6- dione |
| 261 | 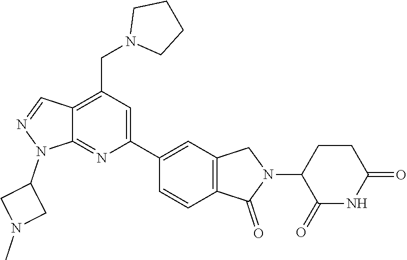 [SMILES: CN1CC(n2ncc3c(CN4CCCC4)cc(-c4ccc5c(c4)CN(C4CCC(=O)NC4=O)C5=O)nc32)C1] | 3-(5-(1-(1-methylazetidin-3-yl)-4- (pyrrolidin-1-ylmethyl)-1H- pyrazolo[3,4-b]pyridin-6-yl)-1- oxoisoindolin-2-yl)piperidine-2,6- dione |
| 262 | 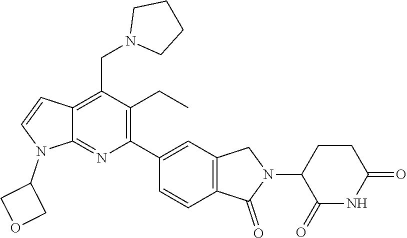 [SMILES: CCc1c(-c2ccc3c(c2)CN(C2CCC(=O)NC2=O)C3=O)nc2c(ccn2C2COC2)c1CN1CCCC1] | 3-(5-(5-ethyl-1-(oxetan-3-yl)-4- (pyrrolidin-1-ylmethyl)-1H- pyrrolo[2,3-b]pyridin-6-yl)-1- oxoisoindolin-2-yl)piperidine-2,6- dione |
| 263 | 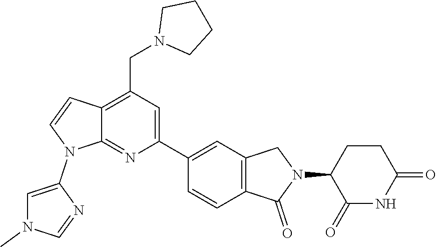 [SMILES: Cn1cnc(-n2ccc3c(CN4CCCC4)cc(-c4ccc5c(c4)CN([C@H]4CCC(=O)NC4=O)C5=O)nc32)c1] | (S)-3-(5-(1-(1-methyl-1H- imidazol-4-yl)-4-(pyrrolidin-1- ylmethyl)-1H-pyrrolo[2,3- b]pyridin-6-yl)-1-oxoisoindolin-2- yl)piperidine-2,6-dione |
| 264 | 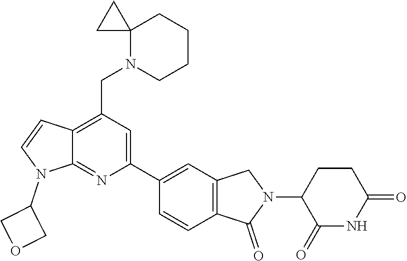 [SMILES: O=C1CCC(N2Cc3cc(-c4cc(CN5CCCCC56CC6)c5ccn(C6COC6)c5n4)ccc3C2=O)C(=O)N1] | 3-(5-(4-((4-azaspiro[2.5]octan-4- yl)methyl)-1-(oxetan-3-yl)-1H- pyrrolo[2,3-b]pyridin-6-yl)-1- oxoisoindolin-2-yl)piperidine-2,6- dione |
| 265 | 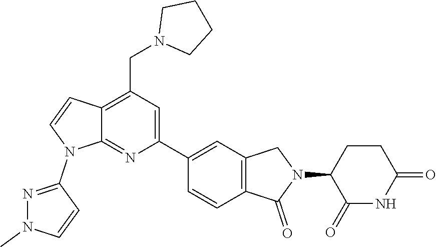 [SMILES: Cn1ccc(-n2ccc3c(CN4CCCC4)cc(-c4ccc5c(c4)CN([C@H]4CCC(=O)NC4=O)C5=O)nc32)n1] | (S)-3-(5-(1-(1-methyl-1H-pyrazol- 3-yl)-4-(pyrrolidin-1-ylmethyl)- 1H-pyrrolo[2,3-b]pyridin-6-yl)-1- oxoisoindolin-2-yl)piperidine-2,6- dione |
| 266 | 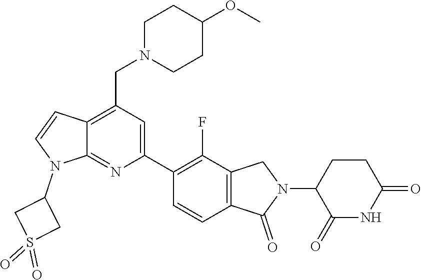 [SMILES: COC1CCN(Cc2cc(-c3ccc4c(c3F)CN(C3CCC(=O)NC3=O)C4=O)nc3c2ccn3C2CS(=O)(=O)C2)CC1] | 3-(5-(1-(1,1-dioxidothietan-3-yl)- 4-((4-methoxypiperidin-1- yl)methyl)-1H-pyrrolo[2,3- b]pyridin-6-yl)-4-fluoro-1- oxoisoindolin-2-yl)piperidine-2,6- dione |
| 267 | 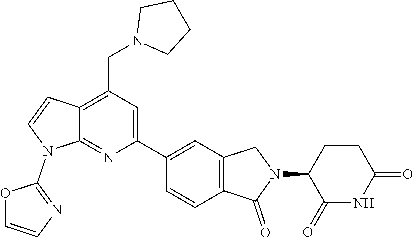 [SMILES: O=C1CC[C@H](N2Cc3cc(-c4cc(CN5CCCC5)c5ccn(-c6ncco6)c5n4)ccc3C2=O)C(=O)N1] | (S)-3-(5-(1-(oxazol-2-yl)-4- (pyrrolidin-1-ylmethyl)-1H- pyrrolo[2,3-b]pyridin-6-yl)-1- oxoisoindolin-2-yl)piperidine-2,6- dione |
| 268 | 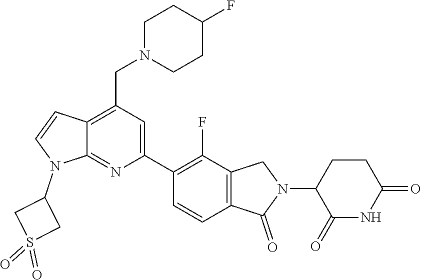 [SMILES: O=C1CCC(N2Cc3c(ccc(-c4cc(CN5CCC(F)CC5)c5ccn(C6CS(=O)(=O)C6)c5n4)c3F)C2=O)C(=O)N1] | 3-(5-(1-(1,1-dioxidothietan-3-yl)- 4-((4-fluoropiperidin-1-yl)methyl)- 1H-pyrrolo[2,3-b]pyridin-6-yl)-4- fluoro-1-oxoisoindolin-2- yl)piperidine-2,6-dione |
| 269 | 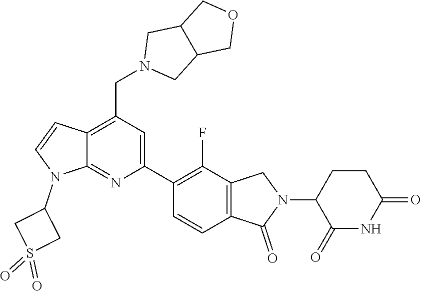 [SMILES: O=C1CCC(N2Cc3c(ccc(-c4cc(CN5CC6COCC6C5)c5ccn(C6CS(=O)(=O)C6)c5n4)c3F)C2=O)C(=O)N1] | 3-(5-(1-(1,1-dioxidothietan-3-yl)- 4-((tetrahydro-1H-furo[3,4- c]pyrrol-5(3H)-yl)methyl)-1H- pyrrolo[2,3-b]pyridin-6-yl)-4- fluoro-1-oxoisoindolin-2- yl)piperidine-2,6-dione |
| 270 | 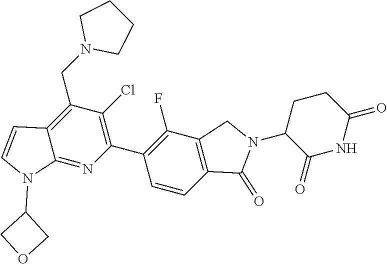 [SMILES: O=C1CCC(N2Cc3c(ccc(-c4nc5c(ccn5C5COC5)c(CN5CCCC5)c4Cl)c3F)C2=O)C(=O)N1] | 3-(5-(5-chloro-1-(oxetan-3-yl)-4- (pyrrolidin-1-ylmethyl)-1H- pyrrolo[2,3-b]pyridin-6-yl)-4- fluoro-1-oxoisoindolin-2- yl)piperidine-2,6-dione |
| 271 | 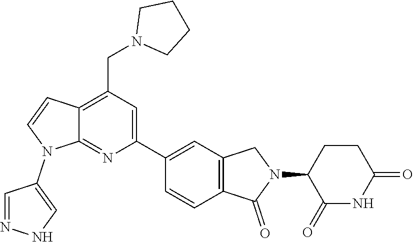 [SMILES: O=C1CC[C@H](N2Cc3cc(-c4cc(CN5CCCC5)c5ccn(-c6cn[nH]c6)c5n4)ccc3C2=O)C(=O)N1] | (S)-3-(5-(1-(1H-pyrazol-4-yl)-4- (pyrrolidin-1-ylmethyl)-1H- pyrrolo[2,3-b]pyridin-6-yl)-1- oxoisoindolin-2-yl)piperidine-2,6- dione |
| 272 | 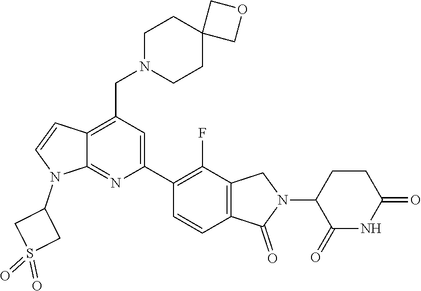 [SMILES: O=C1CCC(N2Cc3c(ccc(-c4cc(CN5CCC6(CC5)COC6)c5ccn(C6CS(=O)(=O)C6)c5n4)c3F)C2=O)C(=O)N1] | 3-(5-(4-((2-oxa-7- azaspiro[3.5]nonan-7-yl)methyl)-1- (1,1-dioxidothietan-3-yl)-1H- pyrrolo[2,3-b]pyridin-6-yl)-4- fluoro-1-oxoisoindolin-2- yl)piperidine-2,6-dione |
| 273 | 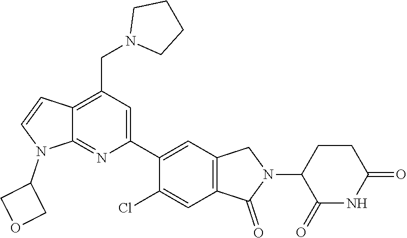 [SMILES: O=C1CCC(N2Cc3cc(-c4cc(CN5CCCC5)c5ccn(C6COC6)c5n4)c(Cl)cc3C2=O)C(=O)N1] | 3-(6-chloro-5-(1-(oxetan-3-yl)-4- (pyrrolidin-1-ylmethyl)-1H- pyrrolo[2,3-b]pyridin-6-yl)-1- oxoisoindolin-2-yl)piperidine-2,6- dione |
| 274 | 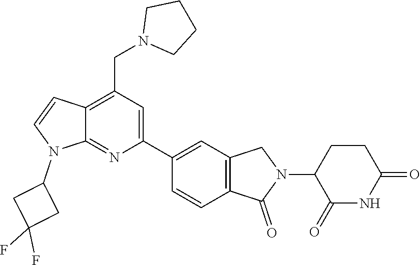 [SMILES: O=C1CCC(N2Cc3cc(-c4cc(CN5CCCC5)c5ccn(C6CC(F)(F)C6)c5n4)ccc3C2=O)C(=O)N1] | 3-(5-(1-(3,3-difluorocyclobutyl)-4- (pyrrolidin-1-ylmethyl)-1H- pyrrolo[2,3-b]pyridin-6-yl)-1- oxoisoindolin-2-yl)piperidine-2,6- dione |
| 275 | 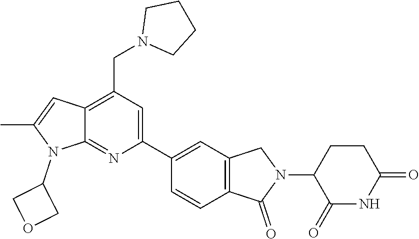 [SMILES: Cc1cc2c(CN3CCCC3)cc(-c3ccc4c(c3)CN(C3CCC(=O)NC3=O)C4=O)nc2n1C1COC1] | 3-(5-(2-methyl-1-(oxetan-3-yl)-4- (pyrrolidin-1-ylmethyl)-1H- pyrrolo[2,3-b]pyridin-6-yl)-1- oxoisoindolin-2-yl)piperidine-2,6- dione |
| 276 | 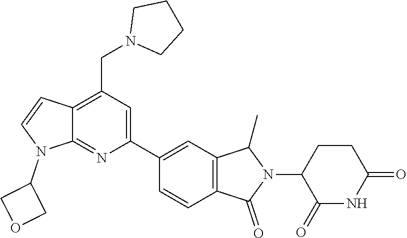 [SMILES: CC1c2cc(-c3cc(CN4CCCC4)c4ccn(C5COC5)c4n3)ccc2C(=O)N1C1CCC(=O)NC1=O] | 3-(3-methyl-5-(1-(oxetan-3-yl)-4- (pyrrolidin-1-ylmethyl)-1H- pyrrolo[2,3-b]pyridin-6-yl)-1- oxoisoindolin-2-yl)piperidine-2,6- dione |
| 277 | 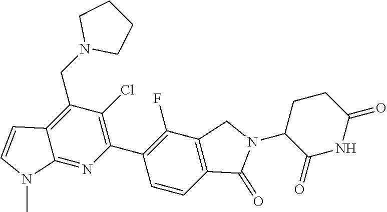 [SMILES: Cn1ccc2c(CN3CCCC3)c(Cl)c(-c3ccc4c(c3F)CN(C3CCC(=O)NC3=O)C4=O)nc21] | 3-(5-(5-chloro-1-methyl-4- (pyrrolidin-1-ylmethyl)-1H- pyrrolo[2,3-b]pyridin-6-yl)-4- fluoro-1-oxoisoindolin-2- yl)piperidine-2,6-dione |
| 278 | 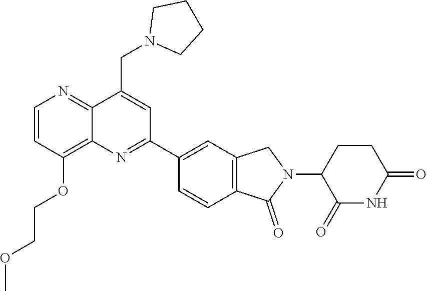 [SMILES: COCCOc1ccnc2c(CN3CCCC3)cc(-c3ccc4c(c3)CN(C3CCC(=O)NC3=O)C4=O)nc12] | 3-(5-(8-(2-methoxyethoxy)-4- (pyrrolidin-1-ylmethyl)-1,5- naphthyridin-2-yl)-1- oxoisoindolin-2-yl)piperidine-2,6- dione |
| 279 | 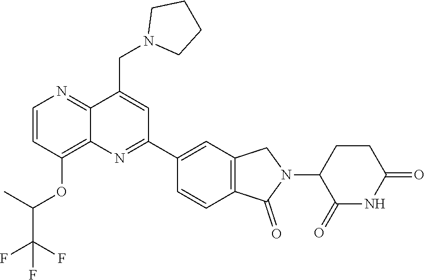 [SMILES: CC(Oc1ccnc2c(CN3CCCC3)cc(-c3ccc4c(c3)CN(C3CCC(=O)NC3=O)C4=O)nc12)C(F)(F)F] | 3-(1-oxo-5-(4-(pyrrolidin-1- ylmethyl)-8-((1,1,1- trifluoropropan-2-yl)oxy)-1,5- naphthyridin-2-yl)isoindolin-2- yl)piperidine-2,6-dione |
| 280 | 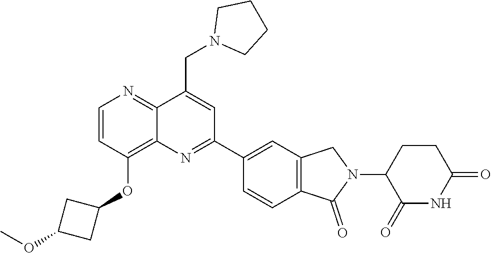 [SMILES: CO[C@H]1C[C@H](Oc2ccnc3c(CN4CCCC4)cc(-c4ccc5c(c4)CN(C4CCC(=O)NC4=O)C5=O)nc23)C1] | rel-3-(5-(8-((1r,3r)-3- methoxycyclobutoxy)-4- (pyrrolidin-1-ylmethyl)-1,5- naphthyridin-2-yl)-1- oxoisoindolin-2-yl)piperidine-2,6- dione |
| 281 | 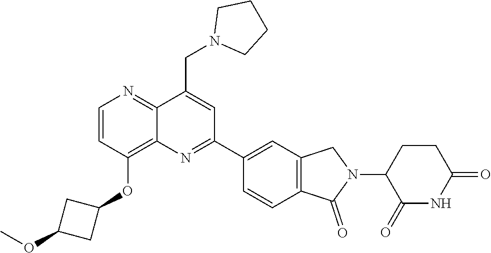 [SMILES: CO[C@H]1C[C@@H](Oc2ccnc3c(CN4CCCC4)cc(-c4ccc5c(c4)CN(C4CCC(=O)NC4=O)C5=O)nc23)C1] | rel-3-(5-(8-((1s,3s)-3- methoxycyclobutoxy)-4- (pyrrolidin-1-ylmethyl)-1,5- naphthyridin-2-yl)-1- oxoisoindolin-2-yl)piperidine-2,6- dione |
| 282 | 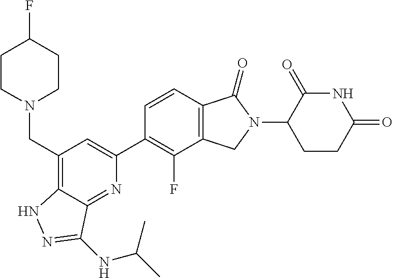 [SMILES: CC(C)Nc1n[nH]c2c(CN3CCC(F)CC3)cc(-c3ccc4c(c3F)CN(C3CCC(=O)NC3=O)C4=O)nc12] | 3-(4-fluoro-5-(7-((4- fluoropiperidin-1-yl)methyl)-3- (isopropylamino)-1H-pyrazolo[4,3- b]pyridin-5-yl)-1-oxoisoindolin-2- yl)piperidine-2,6-dione |
| 283 | 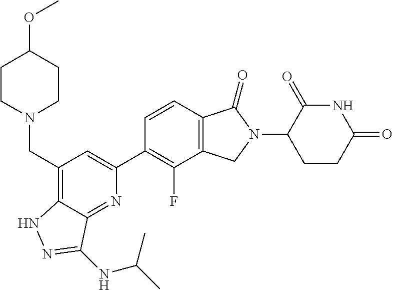 [SMILES: COC1CCN(Cc2cc(-c3ccc4c(c3F)CN(C3CCC(=O)NC3=O)C4=O)nc3c(NC(C)C)n[nH]c23)CC1] | 3-(4-fluoro-5-(3-(isopropylamino)- 7-((4-methoxypiperidin-1- yl)methyl)-1H-pyrazolo[4,3- b]pyridin-5-yl)-1-oxoisoindolin-2- yl)piperidine-2,6-dione |
| 284 | 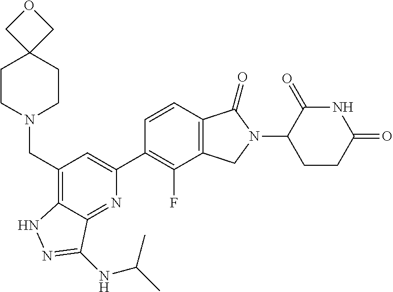 [SMILES: CC(C)Nc1n[nH]c2c(CN3CCC4(CC3)COC4)cc(-c3ccc4c(c3F)CN(C3CCC(=O)NC3=O)C4=O)nc12] | 3-(5-(7-((2-oxa-7- azaspiro[3.5]nonan-7-yl)methyl)-3- (isopropylamino)-1H-pyrazolo[4,3- b]pyridin-5-yl)-4-fluoro-1- oxoisoindolin-2-yl)piperidine-2,6- dione |
| 285 | 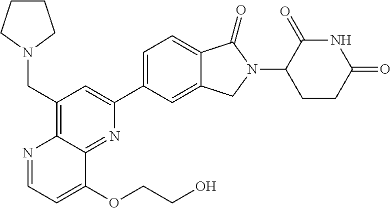 [SMILES: O=C1CCC(N2Cc3cc(-c4cc(CN5CCCC5)c5nccc(OCCO)c5n4)ccc3C2=O)C(=O)N1] | 3-(5-(8-(2-hydroxyethoxy)-4- (pyrrolidin-1-ylmethyl)-1,5- naphthyridin-2-yl)-1- oxoisoindolin-2-yl)piperidine-2,6- dione |
| 286 | 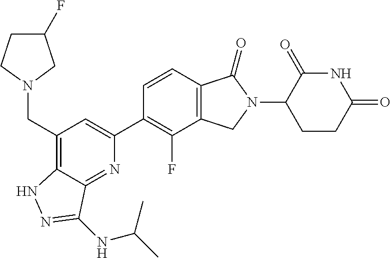 [SMILES: CC(C)Nc1n[nH]c2c(CN3CCC(F)C3)cc(-c3ccc4c(c3F)CN(C3CCC(=O)NC3=O)C4=O)nc12] | 3-(4-fluoro-5-(7-((3- fluoropyrrolidin-1-yl)methyl)-3- (isopropylamino)-1H-pyrazolo[4,3- b]pyridin-5-yl)-1-oxoisoindolin-2- yl)piperidine-2,6-dione |
| 287 | 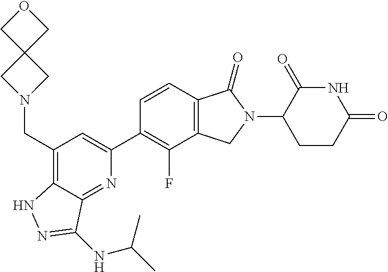 [SMILES: CC(C)Nc1n[nH]c2c(CN3CC4(COC4)C3)cc(-c3ccc4c(c3F)CN(C3CCC(=O)NC3=O)C4=O)nc12] | 3-(5-(7-((2-oxa-6- azaspiro[3.3]heptan-6-yl)methyl)- 3-(isopropylamino)-1H- pyrazolo[4,3-b]pyridin-5-yl)-4- fluoro-1-oxoisoindolin-2- yl)piperidine-2,6-dione |
| 288 | 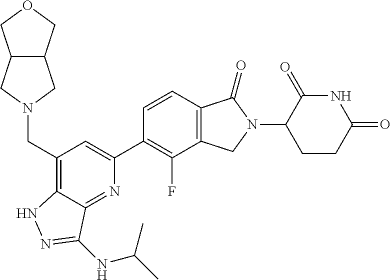 [SMILES: CC(C)Nc1n[nH]c2c(CN3CC4COCC4C3)cc(-c3ccc4c(c3F)CN(C3CCC(=O)NC3=O)C4=O)nc12] | 3-(4-fluoro-5-(3-(isopropylamino)- 7-((tetrahydro-1H-furo[3,4- c]pyrrol-5(3H)-yl)methyl)-1H- pyrazolo[4,3-b]pyridin-5-yl)-1- oxoisoindolin-2-yl)piperidine-2,6- dione |
| 289 | 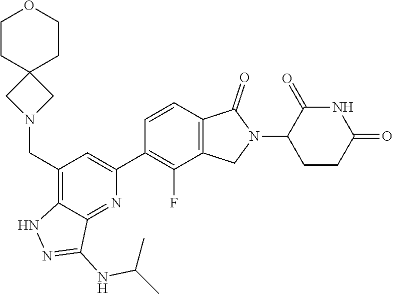 [SMILES: CC(C)Nc1n[nH]c2c(CN3CC4(CCOCC4)C3)cc(-c3ccc4c(c3F)CN(C3CCC(=O)NC3=O)C4=O)nc12] | 3-(5-(7-((7-oxa-2- azaspiro[3.5]nonan-2-yl)methyl)-3- (isopropylamino)-1H-pyrazolo[4,3- b]pyridin-5-yl)-4-fluoro-1- oxoisoindolin-2-yl)piperidine-2,6- dione |
| 290 | 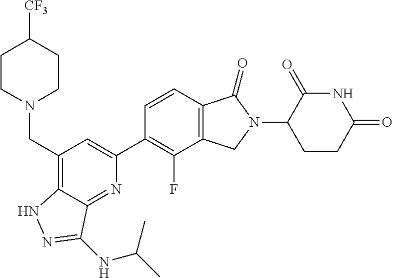 [SMILES: CC(C)Nc1n[nH]c2c(CN3CCC(C(F)(F)F)CC3)cc(-c3ccc4c(c3F)CN(C3CCC(=O)NC3=O)C4=O)nc12] | 3-(4-fluoro-5-(3-(isopropylamino)- 7-((4-(trifluoromethyl)piperidin-1- yl)methyl)-1H-pyrazolo[4,3- b]pyridin-5-yl)-1-oxoisoindolin-2- yl)piperidine-2,6-dione |
| 291 | 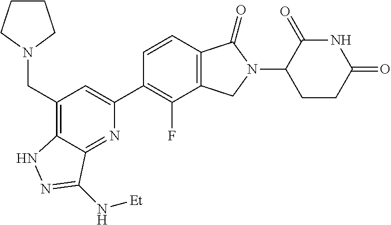 [SMILES: CCNc1n[nH]c2c(CN3CCCC3)cc(-c3ccc4c(c3F)CN(C3CCC(=O)NC3=O)C4=O)nc12] | 3-(5-(3-(ethylamino)-7-(pyrrolidin- 1-ylmethyl)-1H-pyrazolo[4,3- b]pyridin-5-yl)-4-fluoro-1- oxoisoindolin-2-yl)piperidine-2,6- dione |
| 292 | 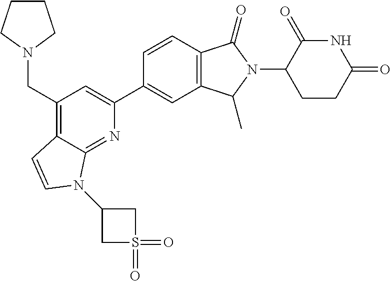 [SMILES: CC1c2cc(-c3cc(CN4CCCC4)c4ccn(C5CS(=O)(=O)C5)c4n3)ccc2C(=O)N1C1CCC(=O)NC1=O] | 3-(5-(1-(1,1-dioxidothietan-3-yl)- 4-(pyrrolidin-1-ylmethyl)-1H- pyrrolo[2,3-b]pyridin-6-yl)-3- methyl-1-oxoisoindolin-2- yl)piperidine-2,6-dione |
| 293 | 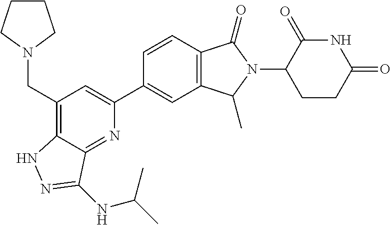 [SMILES: CC(C)Nc1n[nH]c2c(CN3CCCC3)cc(-c3ccc4c(c3)C(C)N(C3CCC(=O)NC3=O)C4=O)nc12] | 3-(5-(3-(isopropylamino)-7- (pyrrolidin-1-ylmethyl)-1H- pyrazolo[4,3-b]pyridin-5-yl)-3- methyl-1-oxoisoindolin-2- yl)piperidine-2,6-dione |
| 294 | 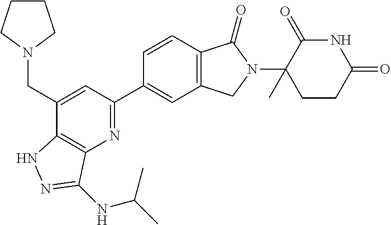 [SMILES: CC(C)Nc1n[nH]c2c(CN3CCCC3)cc(-c3ccc4c(c3)CN(C3(C)CCC(=O)NC3=O)C4=O)nc12] | 3-(5-(3-(isopropylamino)-7- (pyrrolidin-1-ylmethyl)-1H- pyrazolo[4,3-b]pyridin-5-yl)-1- oxoisoindolin-2-yl)-3- methylpiperidine-2,6-dione |
| 295 | 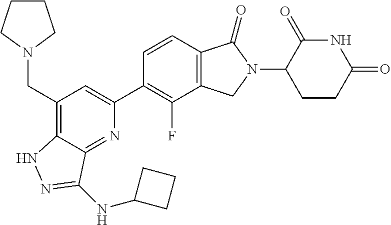 [SMILES: O=C1CCC(N2Cc3c(ccc(-c4cc(CN5CCCC5)c5[nH]nc(NC6CCC6)c5n4)c3F)C2=O)C(=O)N1] | 3-(5-(3-(cyclobutylamino)-7- (pyrrolidin-1-ylmethyl)-1H- pyrazolo[4,3-b]pyridin-5-yl)-4- fluoro-1-oxoisoindolin-2- yl)piperidine-2,6-dione |
| 296 | 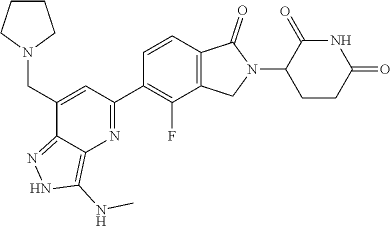 [SMILES: CNc1[nH]nc2c(CN3CCCC3)cc(-c3ccc4c(c3F)CN(C3CCC(=O)NC3=O)C4=O)nc12] | 3-(4-fluoro-5-(3-(methylamino)-7- (pyrrolidin-1-ylmethyl)-2H- pyrazolo[4,3-b]pyridin-5-yl)-1- oxoisoindolin-2-yl)piperidine-2,6- dione |
| 297 | 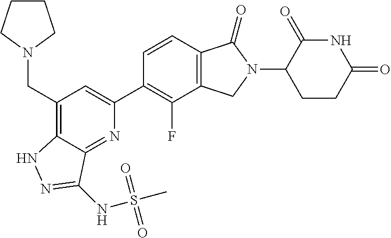 [SMILES: CS(=O)(=O)Nc1n[nH]c2c(CN3CCCC3)cc(-c3ccc4c(c3F)CN(C3CCC(=O)NC3=O)C4=O)nc12] | N-(5-(2-(2,6-dioxopiperidin-3-yl)- 4-fluoro-1-oxoisoindolin-5-yl)-7- (pyrrolidin-1-ylmethyl)-1H- pyrazolo[4,3-b]pyridin-3- yl)methanesulfonamide |
| 299 | 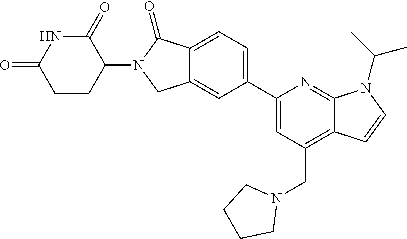 [SMILES: CC(C)n1ccc2c(CN3CCCC3)cc(-c3ccc4c(c3)CN(C3CCC(=O)NC3=O)C4=O)nc21] | 3-(5-(1-isopropyl-4-(pyrrolidin-1- ylmethyl)-1H-pyrrolo[2,3- b]pyridin-6-yl)-1-oxoisoindolin-2- yl)piperidine-2,6-dione |
| 300 | 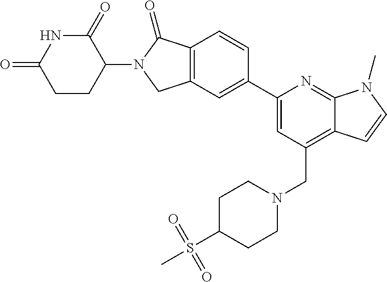 [SMILES: Cn1ccc2c(CN3CCC(S(C)(=O)=O)CC3)cc(-c3ccc4c(c3)CN(C3CCC(=O)NC3=O)C4=O)nc21] | 3-(5-(1-methyl-4-((4- (methylsulfonyl)piperidin-1- yl)methyl)-1H-pyrrolo[2,3- b]pyridin-6-yl)-1-oxoisoindolin-2- yl)piperidine-2,6-dione |
| 301 | 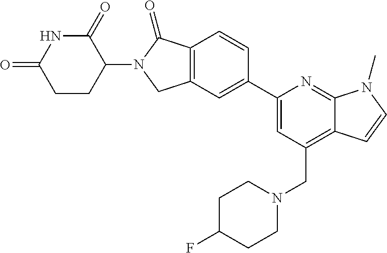 [SMILES: Cn1ccc2c(CN3CCC(F)CC3)cc(-c3ccc4c(c3)CN(C3CCC(=O)NC3=O)C4=O)nc21] | 3-(5-(4-((4-fluoropiperidin-1- yl)methyl)-1-methyl-1H- pyrrolo[2,3-b]pyridin-6-yl)-1- oxoisoindolin-2-yl)piperidine-2,6- dione |
| 302 | 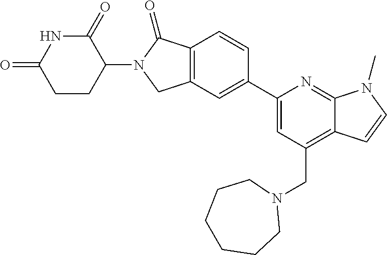 [SMILES: Cn1ccc2c(CN3CCCCCC3)cc(-c3ccc4c(c3)CN(C3CCC(=O)NC3=O)C4=O)nc21] | 3-(5-(4-(azepan-1-ylmethyl)-1- methyl-1H-pyrrolo[2,3-b]pyridin- 6-yl)-1-oxoisoindolin-2- yl)piperidine-2,6-dione |
| 303 | 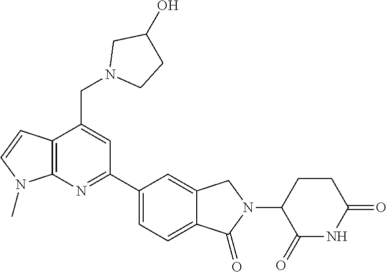 [SMILES: Cn1ccc2c(CN3CCC(O)C3)cc(-c3ccc4c(c3)CN(C3CCC(=O)NC3=O)C4=O)nc21] | 3-(5-(4-((3-hydroxypyrrolidin-1- yl)methyl)-1-methyl-1H- pyrrolo[2,3-b]pyridin-6-yl)-1- oxoisoindolin-2-yl)piperidine-2,6- dione |
| 304 | 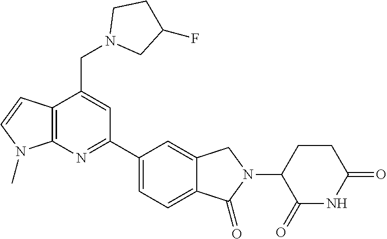 [SMILES: Cn1ccc2c(CN3CCC(F)C3)cc(-c3ccc4c(c3)CN(C3CCC(=O)NC3=O)C4=O)nc21] | 3-(5-(4-((3-fluoropyrrolidin-1- yl)methyl)-1-methyl-1H- pyrrolo[2,3-b]pyridin-6-yl)-1- oxoisoindolin-2-yl)piperidine-2,6- dione |
| 305 | 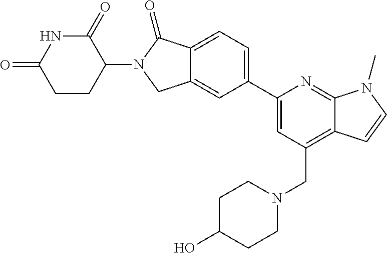 [SMILES: Cn1ccc2c(CN3CCC(O)CC3)cc(-c3ccc4c(c3)CN(C3CCC(=O)NC3=O)C4=O)nc21] | 3-(5-(4-((4-hydroxypiperidin-1- yl)methyl)-1-methyl-1H- pyrrolo[2,3-b]pyridin-6-yl)-1- oxoisoindolin-2-yl)piperidine-2,6- dione |
| 306 | 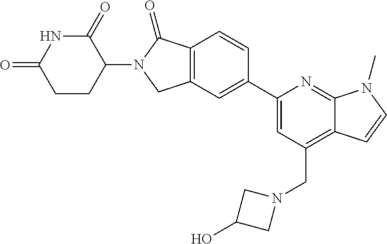 [SMILES: Cn1ccc2c(CN3CC(O)C3)cc(-c3ccc4c(c3)CN(C3CCC(=O)NC3=O)C4=O)nc21] | 3-(5-(4-((3-hydroxyazetidin-1- yl)methyl)-1-methyl-1H- pyrrolo[2,3-b]pyridin-6-yl)-1- oxoisoindolin-2-yl)piperidine-2,6- dione |
| 307 | 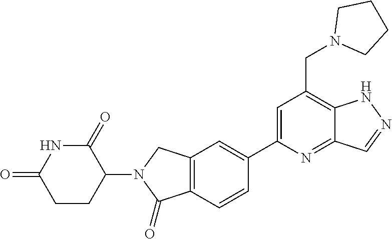 [SMILES: O=C1CCC(N2Cc3cc(-c4cc(CN5CCCC5)c5[nH]ncc5n4)ccc3C2=O)C(=O)N1] | 3-(1-oxo-5-(7-(pyrrolidin-1- ylmethyl)-1H-pyrazolo[4,3- b]pyridin-5-yl)isoindolin-2- yl)piperidine-2,6-dione |
| 308 | 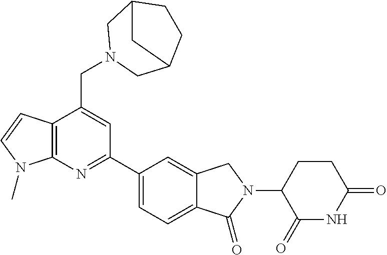 [SMILES: Cn1ccc2c(CN3CC4CCC(C4)C3)cc(-c3ccc4c(c3)CN(C3CCC(=O)NC3=O)C4=O)nc21] | 3-(5-(4-((3-azabicyclo[3.2.1 ]octan- 3-yl)methyl)-1-methyl-1H- pyrrolo[2,3-b]pyridin-6-yl)-1- oxoisoindolin-2-yl)piperidine-2,6- dione |
| 309 | 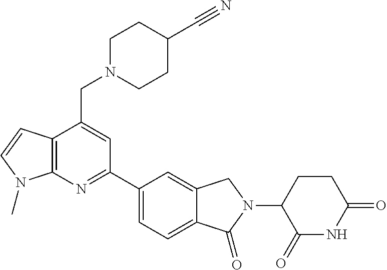 [SMILES: Cn1ccc2c(CN3CCC(C#N)CC3)cc(-c3ccc4c(c3)CN(C3CCC(=O)NC3=O)C4=O)nc21] | 1-((6-(2-(2,6-dioxopiperidin-3-yl)- 1-oxoisoindolin-5-yl)-1-methyl- 1H-pyrrolo[2,3-b]pyridin-4- yl)methyl)piperidine-4-carbonitrile |
| 310 | 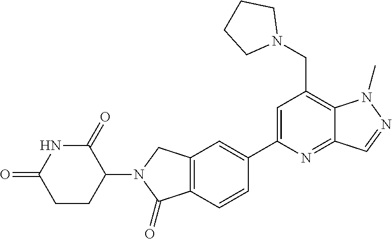 [SMILES: Cn1ncc2nc(-c3ccc4c(c3)CN(C3CCC(=O)NC3=O)C4=O)cc(CN3CCCC3)c21] | 3-(5-(1-methyl-7-(pyrrolidin-1- ylmethyl)-1H-pyrazolo[4,3- b]pyridin-5-yl)-1-oxoisoindolin-2- yl)piperidine-2,6-dione |
| 311 | 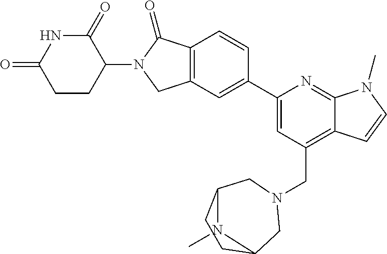 [SMILES: CN1C2CCC1CN(Cc1cc(-c3ccc4c(c3)CN(C3CCC(=O)NC3=O)C4=O)nc3c1ccn3C)C2] | 3-(5-(1-methyl-4-((8-methyl-3,8- diazabicyclo[3.2.1]octan-3- yl)methyl)-1H-pyrrolo[2,3- b]pyridin-6-yl)-1-oxoisoindolin-2- yl)piperidine-2,6-dione |
| 312 | 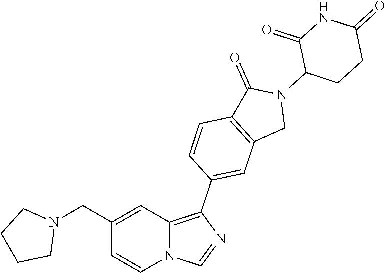 [SMILES: O=C1CCC(N2Cc3cc(-c4ncn5ccc(CN6CCCC6)cc45)ccc3C2=O)C(=O)N1] | 3-(1-oxo-5-(7-(pyrrolidin-1- ylmethyl)imidazo[1,5-a]pyridin-1- yl)isoindolin-2-yl)piperidine-2,6- dione |
| 313 | 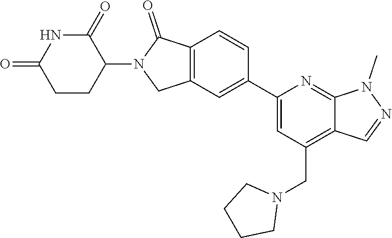 | 3-(5-(1-methyl-4-(pyrrolidin-1- ylmethyl)-1H-pyrazolo[3,4- b]pyridin-6-yl)-1-oxoisoindolin-2- yl)piperidine-2,6-dione |
| 314 | 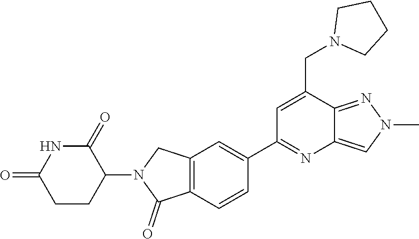 | 3-(5-(2-methyl-7-(pyrrolidin-1- ylmethyl)-2H-pyrazolo[4,3- b]pyridin-5-yl)-1-oxoisoindolin-2- yl)piperidine-2,6-dione |
| 315 | 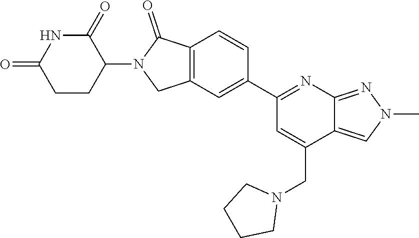 | 3-(5-(2-methyl-4-(pyrrolidin-1- ylmethyl)-2H-pyrazolo[3,4- b]pyridin-6-yl)-1-oxoisoindolin-2- yl)piperidine-2,6-dione |
| 316 | 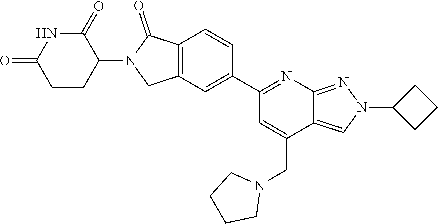 | 3-(5-(2-cyclobutyl-4-(pyrrolidin-1- ylmethyl)-2H-pyrazolo[3,4- b]pyridin-6-yl)-1-oxoisoindolin-2- yl)piperidine-2,6-dione |
| 317 | 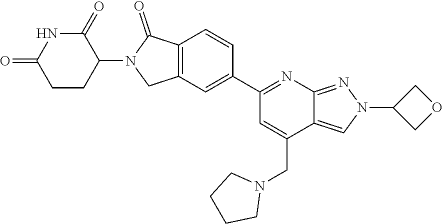 | 3-(5-(2-(oxetan-3-yl)-4-(pyrrolidin- 1-ylmethyl)-2H-pyrazolo[3,4- b]pyridin-6-yl)-1-oxoisoindolin-2- yl)piperidine-2,6-dione |
| 318 | 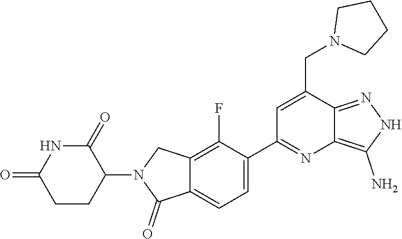 [SMILES: Nc1[nH]nc2c(CN3CCCC3)cc(-c3ccc4c(c3F)CN(C3CCC(=O)NC3=O)C4=O)nc12] | 3-(5-(3-amino-7-(pyrrolidin-1- ylmethyl)-2H-pyrazolo[4,3- b]pyridin-5-yl)-4-fluoro-1- oxoisoindolin-2-yl)piperidine-2,6- dione |

The compounds of the present disclosure may possess advantageous characteristics, as compared to known compounds, such as known IKZF2 degraders. For example, the compounds of the present disclosure may display more potent IKZF2 activity, more favorable pharmacokinetic properties (e.g., as measured by Cₘₐₓ, Tₘₐₓ, and/or AUC), and/or less interaction with other cellular targets (e.g., hepatic cellular transporter such as OATP1B1) and accordingly improved safety (e.g., drug-drug interaction). These beneficial properties of the compounds of the present disclosure may be measured according to methods commonly available in the art, such as methods exemplified herein.

The compounds of the present disclosure may possess advantageous characteristics, as compared to other p300 degraders. For example, the compounds of the present disclosure may potentially show selectivity for IKZF2 over IKZF1, display more potent degradation activity against IKZF2, more favorable pharmacokinetic properties (e.g., as measured by Cₘₐₓ, Tₘₐₓ, and/or AUC), and/or less interaction with other cellular targets (e.g., hepatic cellular transporter such as OATP1B1) and accordingly improved safety (e.g., drug-drug interaction).

In certain embodiments, a compound disclosed herein is “selective” or “shows selectivity” for IKZF2 when it selectively degrades or shows selective degradation of IKZF2 over IKZF1. For example, a compound is selective for IKZF2 when it has a DC50 for IKZF2 that is lower than its DC50 for IKZF1. In certain embodiments, a compound disclosed herein shows selective degradation of IKZF2 over IKZF1 when it has a Dₘₐₓ for IKZF2 that is greater than its Dₘₐₓ for IKZF1. In certain embodiments, a compound disclosed herein shows selective degradation of IKZF2 over IKZF1 through a combination of both lower DC₅₀ and greater Dₘₐₓ for IKZF2, as compared to those for IKZF1. In certain embodiments, a compound disclosed herein shows selectivity when a compound has a DC₅₀ for IKZF2 at least 10-fold lower than its DC₅₀ for IKZF1 and/or a value of Dₘₐₓ for IKZF2 minus Dₘₐₓ for IKZF1 (ΔDₘₐₓ) of at least 30, at least 35, at least 40, or at least 45 percentage points. In certain preferred embodiments, a compound disclosed herein shows selectivity when it has a DC₅₀ for IKZF2 at least 30-fold lower than its DC₅₀ for IKZF1 and/or a value of Dₘₐₓ for IKZF2 minus Dₘₐₓ for IKZF1 (ΔDₘₐₓ) of at least 50, at least 55, at least 60, or at least 65 percentage points. In certain more preferred embodiments, a compound disclosed herein shows selectivity when it has a DC₅₀ for IKZF2 at least 100-fold lower than its DC₅₀ for IKZF1 and/or a value of Dₘₐₓ for IKZF2 minus Dₘₐₓ for IKZF1 (ΔDₘₐₓ) of at least 70, at least 75, at least 80, at least 85, or at least 90 percentage points. These beneficial properties of the compounds of the present disclosure can be measured according to methods commonly available in the art, such as methods exemplified herein.

Due to the existence of double bonds, the compounds of the present disclosure may be in cis or trans, or Z or E, configuration. It is understood that although one configuration may be depicted in the structure of the compounds or formulae of the present disclosure, the present disclosure also encompasses the other configuration. For example, the compounds or formulae of the present disclosure may be depicted in cis or trans, or Z or E, configuration.

In one embodiment, a compound of the present disclosure (e.g., a compound of any of the formulae or any individual compounds disclosed herein) is a pharmaceutically acceptable salt. In another embodiment, a compound of the present disclosure (e.g., a compound of any of the formulae or any individual compounds disclosed herein) is a solvate. In another embodiment, a compound of the present disclosure (e.g., a compound of any of the formulae or any individual compounds disclosed herein) is a hydrate.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Further Forms of Compounds Disclosed Herein

### Pharmaceutically Acceptable Salts

In certain embodiments, the compounds disclosed herein exist as their pharmaceutically acceptable salts. In certain embodiments, the methods disclosed herein include methods of treating diseases by administering such pharmaceutically acceptable salts. In certain embodiments, the methods disclosed herein include methods of treating diseases by administering such pharmaceutically acceptable salts as pharmaceutical compositions.

In certain embodiments, the compounds described herein possess acidic or basic groups and therefor react with any of a number of inorganic or organic bases, and inorganic and organic acids, to form a pharmaceutically acceptable salt. In certain embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, y-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In certain embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N+ (C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In certain embodiments, water or oil-soluble or dispersible products are obtained by such quaternization.

### Solvates

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates are within the scope of the invention.

It will also be appreciated by those skilled in organic chemistry that many organic compounds can exist in more than one crystalline form. For example, crystalline form may vary from solvate to solvate. Thus, all crystalline forms or the pharmaceutically acceptable solvates thereof are contemplated and are within the scope of the present invention.

In certain embodiments, the compounds described herein exist as solvates. The present disclosure provides for methods of treating diseases by administering such solvates. The present disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Isomers/Stereoisomers

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

In certain embodiments, the compounds described herein exist as geometric isomers. In certain embodiments, the compounds described herein possess one or more double bonds. The compounds disclosed herein include all cis, trans, syn, anti, entgegen (E), and zusammen (Z) isomers as well as the corresponding mixtures thereof. All geometric forms of the compounds disclosed herein are contemplated and are within the scope of the invention.

In certain embodiments, the compounds disclosed herein possess one or more chiral centers and each center exists in the R configuration or S configuration. The compounds disclosed herein include all diastereomeric, enantiomeric, and epimeric forms as well as the corresponding mixtures thereof. All diastereomeric, enantiomeric, and epimeric forms of the compounds disclosed herein are contemplated and are within the scope of the invention.

In additional embodiments of the compounds and methods provided herein, mixtures of enantiomers and/or diastereoisomers, resulting from a single preparative step, combination, or interconversion are useful for the applications described herein. In certain embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In certain embodiments, dissociable complexes are preferred. In certain embodiments, the diastereomers have distinct physical properties (e.g., melting points, boiling points, solubilities, reactivity, etc.) and are separated by taking advantage of these dissimilarities. In certain embodiments, the diastereomers are separated by chiral chromatography, or preferably, by separation/resolution techniques based upon differences in solubility. In certain embodiments, the optically pure enantiomer is then recovered, along with the resolving agent.

### Tautomers

In certain embodiments, compounds described herein exist as tautomers. The compounds described herein include all possible tautomers within the formulas described herein.

Tautomers are compounds that are interconvertible by migration of a hydrogen atom, accompanied by a switch of a single bond and an adjacent double bond. In bonding arrangements where tautomerization is possible, a chemical equilibrium of the tautomers will exist. All tautomeric forms of the compounds disclosed herein are contemplated and are within the scope of the invention. The exact ratio of the tautomers depends on several factors, including temperature, solvent, and pH.

### Pharmaceutical Compositions

In certain embodiments, the compound described herein is administered as a pure chemical. In certain embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, the present disclosure provides pharmaceutical compositions comprising a compound described herein, or a pharmaceutically acceptable salt, solvate, or stereoisomer thereof, and a pharmaceutically acceptable excipient.

In certain embodiments, the compound provided herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In certain embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In certain embodiments, the pharmaceutical composition is formulated for intravenous injection, oral administration, inhalation, nasal administration, topical administration, or ophthalmic administration. In certain embodiments, the pharmaceutical composition is formulated for oral administration. In certain embodiments, the pharmaceutical composition is formulated for intravenous injection. In certain embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In certain embodiments, the pharmaceutical composition is formulated as a tablet.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached. In certain embodiments, the present method involves the administration of about 0.1 μg to about 50 mg of at least one compound described herein per kg body weight of the subject. For a 70 kg patient, dosages of from about 10 μg to about 200 mg of the compound disclosed herein would be more commonly used, depending on a subject's physiological response.

By way of example only, the dose of the compound described herein for methods of treating a disease as described herein is about 0.001 to about 1 mg/kg body weight of the subject per day, for example, about 0.001 mg, about 0.002 mg, about 0.005 mg, about 0.010 mg, 0.015 mg, about 0.020 mg, about 0.025 mg, about 0.050 mg, about 0.075 mg, about 0.1 mg, about 0.15 mg, about 0.2 mg, about 0.25 mg, about 0.5 mg, about 0.75 mg, or about 1 mg/kg body weight per day. In certain embodiments, the dose of compound described herein for the described methods is about 1 to about 1000 mg/kg body weight of the subject being treated per day, for example, about 1 mg, about 2 mg, about 5 mg, about 10 mg, about 15 mg, about 20 mg, about 25 mg, about 50 mg, about 75 mg, about 100 mg, about 150 mg, about 200 mg, about 250 mg, about 500 mg, about 750 mg, or about 1000 mg per day.

### Preparation of the Compounds

The compounds of the present disclosure can be prepared in a number of ways well known to those skilled in the art of organic synthesis. By way of example, the compounds of the present disclosure can be synthesized using the methods described below, together with synthetic methods known in the art of synthetic organic chemistry, or variations thereon as appreciated by those skilled in the art. The compounds of the present disclosure (i.e., a compound of the present application (e.g., a compound of any of the formulae or any individual compounds disclosed herein)) can be synthesized by following the general synthetic scheme below as well as the steps outlined in the examples, schemes, procedures, and/or synthesis described herein (e.g., Examples).

### General Synthetic Schemes

Exemplary compounds can be prepared by following the general synthetic procedures as outlined in the following schemes.

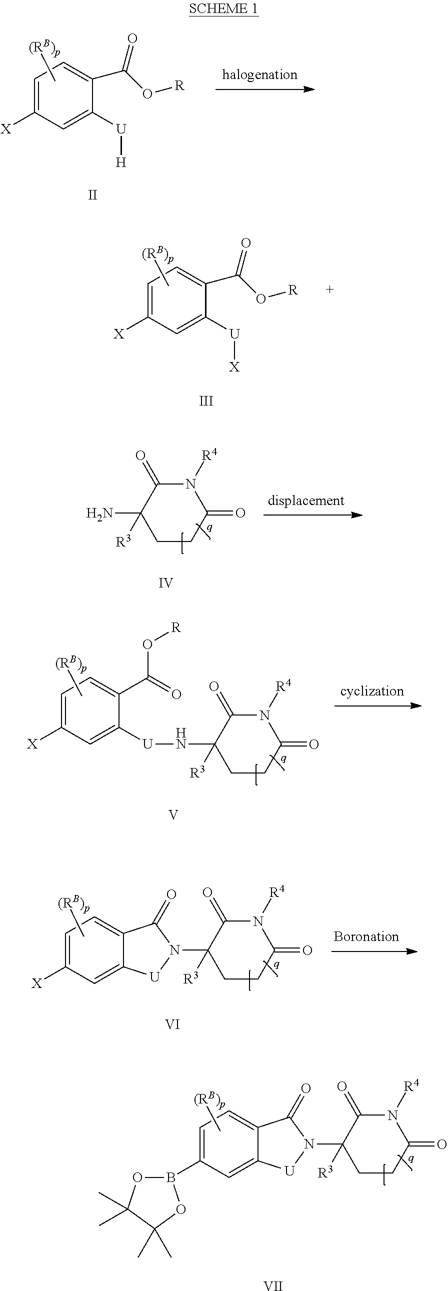

According to SCHEME 1, commercially available or synthetically accessible substituted phenyl carboxylate of formula (II, R is C₁₋₆ alkyl, X is halogen) is reacted with a radical initiator, such as dibenzoyl peroxide (BPO), azobisisobutyronitrile (AIBN) or the like; in the presence of a halogen source such as N-bromosuccinimide (NBS), N-chlorosuccinimide (NCS) and the like; in a suitable solvent such as CCl₄, benzene, or the like; at temperatures ranging from 60° C. to 100° C.; to provide halogenated substituted phenyl carboxylate of formula (III, R is C₁₋₆ alkyl, X is halogen). Halogenated substituted phenyl carboxylate of formula III is treated with a commercially available or synthetically accessible amino glutarimide of formula IV, in the presence of a suitable base such as DIPEA, TEA and the like; in an aprotic solvent such as MeCN, DMF, or the like; at temperatures ranging from 0° C. to 25° C., preferably 10° C.; to provide a compound of formula (V). A compound of formula (V) is cyclized in the presence of a suitable acid such as AcOH, TFA or the like; in a suitable solvent such as dichloromethane (DCM), dichloroethane (DCE) or the like; at temperatures ranging from 25° C. to 80° C., preferably 60° C.; to afford a cyclized compound of formula (VI). A compound of formula (VI) is coupled with commercially available 4,4,4′,4′,5,5,5′,5′— octamethyl-2,2′-bi (1,3,2-dioxaborolane) under palladium catalyzed boronation conditions; with a suitable catalyst such as Pd(dppf)Cl₂, Pd (OAc)₂, or the like; a suitable base such a K₃PO₄, Cs₂CO₃, KOAc, or the like; in a suitable solvent such as dioxane, DMF, THE, or the like; at temperatures ranging from 60° C. to about 120° C.; to provide boronic ester compound of formula (VII).

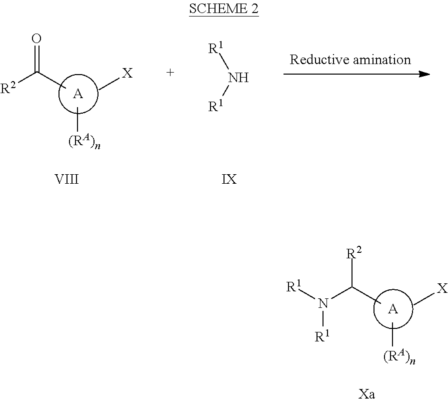

According to SCHEME 2, commercially available or synthetically accessible aldehyde or ketone of formula (VIII, X=halogen) is coupled with a commercially available or synthetically accessible amine compound of formula (IX), in the presence of a suitable reductant such as NaBCNH₃, NaBH(OAc)₃, or the like; in an alcoholic solvent such as MeOH, EtOH, or the like; at temperatures ranging from 0° C. to about 50° C., preferably 25° C.; to afford a compound of formula (Xa).

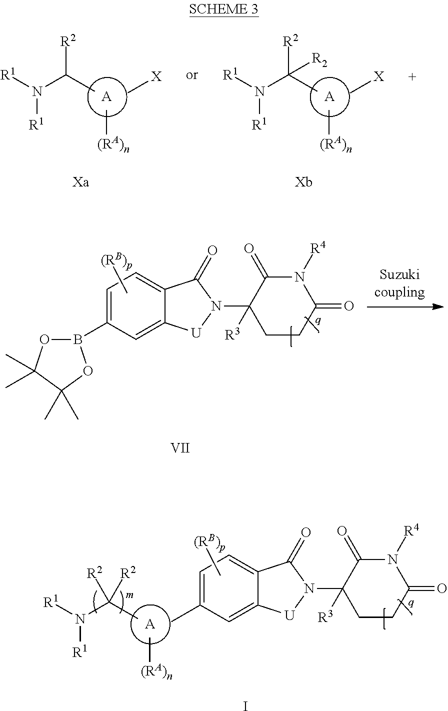

According to SCHEME 3, commercially available or synthetically accessible aldehyde or ketone of formula (VIII, X=halogen) is reacted with a commercially available or synthetically accessible Grignard reagent of formula (XI), in a solvent such as diethyl ether, THE, or the like; at temperatures ranging from −20° C. to about 25° C., preferably 0° C.; to afford an alcohol compound of formula (XII); a compound of formula (XII) is reacted with a mesylating agent such as MsCl or the like; in the presence of a suitable base such as DIPEA, TEA, or the like; in a solvent such as DCM, THE, or the like; at temperatures ranging from 10° C. to about 30° C., preferably 0° C.; to afford a compound of formula (XIII). A compound of formula (XIII) was reacted with commercially available or synthetically accessible amine of formula (IX), in the presence of a suitable base such as Cs₂CO₃, K₂CO₃, or the like; in a solvent such as DMF, DMSO, or the like; at temperatures ranging from 25° C. to about 100° C., preferably 80° C.; to afford a compound of formula (Xb).

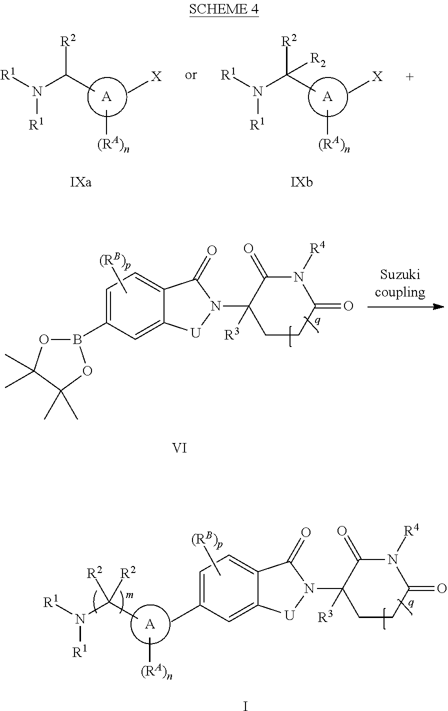

According to SCHEME 4, a brononic ester compound of formula (VII) is reacted with either halogenated compound of formula (Xa) or (Xb) under Suzuki coupling conditions employing a suitable catalyst such as Pd(Ph₃P)₄, Pd₂(dba)₃, Pd (ddpf) C₁₂, or the like; a suitable base such a K₃PO₄, Cs₂CO₃, or the like; in a suitable solvent such as dioxane, DMF, or the like; with a co-solvent such as water; at temperatures ranging from 60° C. to about 120° C., preferably 80° C.; to afford a claimed compound of formula (I).

Those skilled in the art will recognize if a stereocenter exists in the compounds of the present disclosure (e.g., a compound of any of the formulae or any individual compounds disclosed herein). Accordingly, the present disclosure includes both possible stereoisomers (unless specified in the synthesis) and includes not only racemic compound but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Analytical Methods, Materials, and Instrumentation

Unless otherwise noted, reagents and solvents were used as received from commercial suppliers. Proton nuclear magnetic resonance (NMR) spectra were obtained on either Bruker or Varian spectrometers at 400 MHz. Spectra are given in ppm (δ) and coupling constants, J, are reported in Hertz. Tetramethylsilane (TMS) was used as an internal standard. Liquid chromatography-mass spectrometry (LC/MS) were collected using a SHIMADZU LCMS-2020EV or Agilent 1260-6125B LCMS. Purity and low resolution mass spectral data were measured using Agilent 1260-6125B LCMS system (with Diode Array Detector, and Agilent G6125BA Mass spectrometer) or using Waters Acquity UPLC system (with Diode Array Detector, and Waters 3100 Mass Detector). The purity was characterized by UV wavelength 214 nm, 220 nm, 254 nm and ESI. Column: poroshell 120 EC-C18 2.7 μm 4.6×100 mm; Flow rate 0.8 mL/min; Solvent A (100/0.1 water/formic acid), Solvent B (100 acetonitrile); gradient: hold 5% B to 0.3 min, 5-95% B from 0.3 to 2 min, hold 95% B to 4.8 min, 95-5% B from 4.8 to 5.4 min, then hold 5% B to 6.5 min. Or, column: Acquity UPLC BEH C18 1.7 μm 2.1×50 mm; Flow rate 0.5 mL/min; Solvent A (0.1% formic acid water), Solvent B (acetonitrile); gradient: hold 5% B for 0.2 min, 5-95% B from 0.2 to 2.0 min, hold 95% B to 3.1 min, then 5% B at 3.5 min.

### Biological Assays

The biological activities of the compounds of the present application can be assessed with methods and assays known in the art.

The binding potency of the compounds to CRBN/DDB1 is determined using HTRF assay technology. HTRF signals are measured by displacing Cy5-labeled thalidomide with the testing compounds to His tagged CRBN+DDB-DLS7+CXU4. Data is analyzed using XLfit using four parameters dose response curve to determine IC₅₀S.

The cellular degradation activity of IKZF2 is measued by FACS in Jurkat cells with the testing compound concentrations from 0.001, 0.01, 0.1, 1 to 10 μM for 24 hrs. The protein concentration is assessed by PVDF membranes and immunoblot with antibodies against IKZF2. Band intensities are quantified and analyzed using XLfit.

Alternatively, the cellular degradation activity of IKZF2 is measued by FACS in Jurkat cells with the testing compound concentrations from 0.05 to 10 μM for 24 hrs. Cells are stained with IKZF2 primary antibody and secondary antibodies followed by imaged on iQue Flowcytometer and IKZF2 levels are quantified using iQue software.

Alternatively, the cellular degradation activity of IKZF2 is measured by HiBit IKZF2 assay with the HiBiT protein tagging system applying to modified HEK293T Flp-in-HiBiT cells. Test and reference compounds are diluted from 1 μM at 3 folds for 11 doses. The Nano-Glo® HiBiT lytic detection system is utilized for detecting bioluminescence of the HiBiT tag in treated cells to determine abundance of the tag is proportionate to the level of luminescence. Following normalization to DMSO, dose-response curves are plotted (GraphPad Prism) to determine the concentration points at which 50% of HiBiT-Helios degradation is achieved by each compound.

Alternatively, the cellular degradation activity of IKZF1 is measured by HiBit IKZF1 assay with the HiBiT protein tagging system applying to modified HEK293T Flp-in-HiBiT cells. Test and reference compounds are diluted from 1 μM at 3 folds for 11 doses. The Nano-Glo®HiBiT lytic detection system is utilized for detecting bioluminescence of the HiBiT tag in treated cells to determine abundance of the tag is proportionate to the level of luminescence. Following normalization to DMSO, dose-response curves are plotted (GraphPad Prism) to determine the concentration points at which 50% of HiBiT-Ikaros degradation is achieved by each compound.

### Methods of Use

In certain aspects, the present disclosure provides methods of degrading a IKZF2 protein in a subject, comprising administering to the subject a compound disclosed herein.

In certain aspects, the present disclosure provides uses of a compound disclosed herein in the manufacture of a medicament for degrading a IKZF2 protein in a subject.

In certain aspects, the present disclosure provides compounds disclosed herein for use in degrading a IKZF2 protein in a subject.

In certain aspects, the present disclosure provides methods of treating or preventing a disease or disorder in a subject in need thereof, comprising administering to the subject a compound disclosed herein (e.g., in a therapeutically effective amount).

In certain aspects, the present disclosure provides methods of treating a disease or disorder in a subject in need thereof, comprising administering to the subject a compound disclosed herein (e.g., in a therapeutically effective amount).

In certain aspects, the present disclosure provides uses of a compound disclosed herein in the manufacture of a medicament for treating or preventing a disease or disorder in a subject in need thereof.

In certain aspects, the present disclosure provides uses of a compound disclosed herein in the manufacture of a medicament for treating a disease or disorder in a subject in need thereof.

In certain aspects, the present disclosure provides compounds disclosed herein for use in treating or preventing a disease or disorder in a subject in need thereof.

In certain aspects, the present disclosure provides compounds disclosed herein for use in treating a disease or disorderin a subject in need thereof.

In certain embodiments, the disease or disorder is an IKZF2-mediated disease or disorder.

In certain embodiments, the disease or disorder is selected from T cell leukemia, T cell lymphoma, Hodgkin's lymphoma, non-Hodgkin's lymphoma, myeloid leukemia, non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST).

In certain aspects, the present disclosure provides methods of (a) increasing IL-2 production; (b) suppressing regulatory T cells; (c) enhancing effector T cells; (d) inhibiting tumor growth; and/or (e) enhancing tumor regression in a subject, comprising administering to the subject in need thereof a compound disclosed herein.

In certain aspects, the present disclosure provides uses of a compound disclosed herein in the manufacture of a medicament for (a) increasing IL-2 production; (b) suppressing regulatory T cells; (c) enhancing effector T cells; (d) inhibiting tumor growth; and/or (e) enhancing tumor regression in a subject.

In certain embodiments, the subject is a mammal.

In certain embodiments, the subject is a human.

### Definitions

As used in the specification and appended claims, unless specified to the contrary, the following terms have the meaning indicated below.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley &amp; Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPFC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. F. Eliel, Ed., Univ. of Notre Dame Press, Notre Dame, IN 1972).

The invention additionally encompasses compounds described herein as individual isomers substantially free of other isomers, and alternatively, as mixtures of various isomers.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁-5, C₁₋₄, C₁₋₃, C₁₋₂, C₂₋₆, C₂₋₅, C₂₄, C₂₋₃, C₃₋₆, C₃₋₅, C₃₄, C₄₋₆, C₄₋₅, and C₅₋₆ alkyl.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e., at least one) of the grammatical objects of the article. By way of example “an analogue” means one analogue or more than one analogue.

“Alkyl” as used herein, refers to a radical of a straight-chain or branched saturated hydrocarbon group having from 1 to 20 carbon atoms (“C₁₋₂₀ alkyl”). In certain embodiments, an alkyl group has 1 to 12 carbon atoms (“C₁₋₁₂ alkyl”). In certain embodiments, an alkyl group has 1 to 10 carbon atoms (“C₁₋₁₀ alkyl”). In certain embodiments, an alkyl group has 1 to 9 carbon atoms (“C₁₋₉ alkyl”). In certain embodiments, an alkyl group has 1 to 8 carbon atoms (“C₁₋₈ alkyl”). In certain embodiments, an alkyl group has 1 to 7 carbon atoms (“C₁₋₇ alkyl”). In certain embodiments, an alkyl group has 1 to 6 carbon atoms (“C₁₋₆ alkyl”, which is also referred to herein as “lower alkyl”). In certain embodiments, an alkyl group has 1 to 5 carbon atoms (“C₁-5 alkyl”). In certain embodiments, an alkyl group has 1 to 4 carbon atoms (“C₁₋₄ alkyl”). In certain embodiments, an alkyl group has 1 to 3 carbon atoms (“C₁₋₃ alkyl”). In certain embodiments, an alkyl group has 1 to 2 carbon atoms (“C₁₋₂ alkyl”). In certain embodiments, an alkyl group has 1 carbon atom (“C₁ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), i-Pr (—CH (CH₃)₂), n-Pr (—CH₂CH₂CH₃), n-Bu (—CH₂CH₂CH₂CH₃), or i-Bu (—CH₂CH (CH₃)₂).

“Alkylene” as used herein, refers to an alkyl group wherein two hydrogens are removed to provide a divalent radical. When a range or number of carbons is provided for a particular “alkylene” group, it is understood that the range or number refers to the range or number of carbons in the linear carbon divalent chain. An “alkylene” group may be substituted or unsubstituted with one or more substituents as described herein. Exemplary unsubstituted divalent alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted divalent alkylene groups, e.g., substituted with one or more alkyl (methyl) groups, include but are not limited to, substituted methylene (—CH (CH₃)—, (—C(CH₃)₂—), substituted ethylene (—CH (CH₃) CH₂—, —CH₂CH (CH₃)—, —C(CH₃)₂CH₂—, —CH₂C(CH₃)₂—), substituted propylene (—CH (CH₃) CH₂CH₂—, —CH₂CH (CH₃) CH₂—, —CH₂CH₂CH (CH₃)—, —C(CH₃)₂CH₂CH₂—, —CH₂C(CH₃)₂CH₂—, —CH₂CH₂C(CH₃)₂—), and the like.

“Alkenyl” as used herein, refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In certain embodiments, an alkenyl group has 2 to 10 carbon atoms (“C₂₋₁₀ alkenyl”). In certain embodiments, an alkenyl group has 2 to 9 carbon atoms (“C₂-9 alkenyl”). In certain embodiments, an alkenyl group has 2 to 8 carbon atoms (“C₂₋₈ alkenyl”). In certain embodiments, an alkenyl group has 2 to 7 carbon atoms (“C₂-7 alkenyl”). In certain embodiments, an alkenyl group has 2 to 6 carbon atoms (“C₂₋₆ alkenyl”). In certain embodiments, an alkenyl group has 2 to 5 carbon atoms (“C₂₋₅ alkenyl”). In certain embodiments, an alkenyl group has 2 to 4 carbon atoms (“C₂₋₄ alkenyl”). In certain embodiments, an alkenyl group has 2 to 3 carbon atoms (“C₂₋₃ alkenyl”). In certain embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” as used herein, refers to an alkenyl group wherein two hydrogens are removed to provide a divalent radical. When a range or number of carbons is provided for a particular “alkenylene” group, it is understood that the range or number refers to the range or number of carbons in the linear carbon divalent chain. An “alkenylene” group may be substituted or unsubstituted with one or more substituents as described herein. Exemplary unsubstituted divalent alkenylene groups include, but are not limited to, ethenylene (—CH═CH—) and propenylene (e.g., —CH═CHCH₂—, —CH₂—CH═CH—). Exemplary substituted divalent alkenylene groups, e.g., substituted with one or more alkyl (methyl) groups, include but are not limited to, substituted ethylene (—C(CH₃)—CH—, —CH═C(CH₃)—), substituted propylene (e.g., —C(CH₃)═CHCH₂—, —CH═C(CH₃) CH₂—, —CH—CHCH (CH₃)—, —CH—CHC(CH₃)₂—, —CH (CH₃)—CH—CH—, —C(CH₃)₂—CH═CH—, —CH₂—C(CH₃)—CH—, —CH₂—CH═C(CH₃)—), and the like.

“Alkynyl” as used herein, refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In certain embodiments, an alkynyl group has 2 to 10 carbon atoms (“C₂₋₁₀ alkynyl”). In certain embodiments, an alkynyl group has 2 to 9 carbon atoms (“C₂₋₉ alkynyl”). In certain embodiments, an alkynyl group has 2 to 8 carbon atoms (“C₂-8 alkynyl”). In certain embodiments, an alkynyl group has 2 to 7 carbon atoms (“C₂-7 alkynyl”). In certain embodiments, an alkynyl group has 2 to 6 carbon atoms (“C₂₋₆ alkynyl”). In certain embodiments, an alkynyl group has 2 to 5 carbon atoms (“C₂₋₅ alkynyl”). In certain embodiments, an alkynyl group has 2 to 4 carbon atoms (“C₂₋₄ alkynyl”). In certain embodiments, an alkynyl group has 2 to 3 carbon atoms (“C₂₋₃ alkynyl”). In certain embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Alkynylene” as used herein, refers to a linear alkynyl group wherein two hydrogens are removed to provide a divalent radical. When a range or number of carbons is provided for a particular “alkynylene” group, it is understood that the range or number refers to the range or number of carbons in the linear carbon divalent chain. An “alkynylene” group may be substituted or unsubstituted with one or more substituents as described herein. Exemplary divalent alkynylene groups include, but are not limited to, substituted or unsubstituted ethynylene, substituted or unsubstituted propynylene, and the like.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In certain embodiments, a heteroalkyl group is a saturated group having 1 to 9 carbon atoms and 1, 2, 3, or 4 heteroatoms (“heteroC₁₋₉ alkyl”). In certain embodiments, a heteroalkyl group is a saturated group having 1 to 8 carbon atoms and 1, 2, 3, or 4 heteroatoms (“heteroC₁₋₈ alkyl”). In certain embodiments, a heteroalkyl group is a saturated group having 1 to 7 carbon atoms and 1, 2, 3, or 4 heteroatoms (“heteroC₁₋₇ alkyl”). In certain embodiments, a heteroalkyl group is a group having 1 to 6 carbon atoms and 1, 2, or 3 heteroatoms (“heteroC₁₋₆ alkyl”). In certain embodiments, a heteroalkyl group is a saturated group having 1 to 5 carbon atoms and 1 or 2 heteroatoms (“heteroC₁-5 alkyl”). In certain embodiments, a heteroalkyl group is a saturated group having 1 to 4 carbon atoms and/or 2 heteroatoms (“heteroC₁₋₄ alkyl”). In certain embodiments, a heteroalkyl group is a saturated group having 1 to 3 carbon atoms and 1 heteroatom (“heteroC₁₋₃ alkyl”). In certain embodiments, a heteroalkyl group is a saturated group having 1 to 2 carbon atoms and 1 heteroatom (“heteroC₁₋₂ alkyl”). In certain embodiments, a heteroalkyl group is a saturated group having 1 carbon atom and 1 heteroatom (“heteroC₁ alkyl”). In certain embodiments, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“heteroC₂₋₁₀ alkenyl”). In certain embodiments, a heteroalkenyl group has 2 to 9 carbon atoms at least one double bond, and 1, 2, 3, or 4 heteroatoms (“heteroC₂₋₉ alkenyl”). In certain embodiments, a heteroalkenyl group has 2 to 8 carbon atoms, at least one double bond, and 1, 2, 3, or 4 heteroatoms (“heteroC₂₋₈ alkenyl”). In certain embodiments, a heteroalkenyl group has 2 to 7 carbon atoms, at least one double bond, and 1, 2, 3, or 4 heteroatoms (“heteroC₂-7 alkenyl”). In certain embodiments, a heteroalkenyl group has 2 to 6 carbon atoms, at least one double bond, and 1, 2, or 3 heteroatoms (“heteroC₂₋₆ alkenyl”). In certain embodiments, a heteroalkenyl group has 2 to 5 carbon atoms, at least one double bond, and 1 or 2 heteroatoms (“heteroC₂₋₅ alkenyl”). In certain embodiments, a heteroalkenyl group has 2 to 4 carbon atoms, at least one double bond, and lor 2 heteroatoms (“heteroC₂₋₄ alkenyl”). In certain embodiments, a heteroalkenyl group has 2 to 3 carbon atoms, at least one double bond, and 1 heteroatom (“heteroC₂₋₃ alkenyl”). In certain embodiments, a heteroalkenyl group has 2 to 6 carbon atoms, at least one double bond, and 1 or 2 heteroatoms (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “heteroalkynyl,” as used herein, refers to an alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms are inserted between a carbon atom and the parent molecule, i.e., between the point of attachment. In certain embodiments, a heteroalkynyl group refers to a group having from 2 to 10 carbon atoms, at least one triple bond, and 1, 2, 3, or 4 heteroatoms (“heteroC₂₋₁₀ alkynyl”). In certain embodiments, a heteroalkynyl group has 2 to 9 carbon atoms, at least one triple bond, and 1, 2, 3, or 4 heteroatoms (“heteroC₂₋₉ alkynyl”). In certain embodiments, a heteroalkynyl group has 2 to 8 carbon atoms, at least one triple bond, and 1, 2, 3, or 4 heteroatoms (“heteroC₂₋₈ alkynyl”). In certain embodiments, a heteroalkynyl group has 2 to 7 carbon atoms, at least one triple bond, and 1, 2, 3, or 4 heteroatoms (“heteroC₂-7 alkynyl”). In certain embodiments, a heteroalkynyl group has 2 to 6 carbon atoms, at least one triple bond, and 1, 2, or 3 heteroatoms (“heteroC₂₋₆ alkynyl”). In certain embodiments, a heteroalkynyl group has 2 to 5 carbon atoms, at least one triple bond, and 1 or 2 heteroatoms (“heteroC₂₋₅ alkynyl”). In certain embodiments, a heteroalkynyl group has 2 to 4 carbon atoms, at least one triple bond, and lor 2 heteroatoms (“heteroC₂₋₄ alkynyl”). In certain embodiments, a heteroalkynyl group has 2 to 3 carbon atoms, at least one triple bond, and 1 heteroatom (“heteroC₂₋₃ alkynyl”). In certain embodiments, a heteroalkynyl group has 2 to 6 carbon atoms, at least one triple bond, and 1 or 2 heteroatoms (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

Analogous to “alkylene,” “alkenylene,” and “alkynylene” as defined above, “heteroalkylene,” “heteroalkenylene,” and “heteroalkynylene,” as used herein, refer to a divalent radical of heteroalkyl, heteroalkenyl, and heteroalkynyl group respectively. When a range or number of carbons is provided for a particular “heteroalkylene,” “heteroalkenylene,” or “heteroalkynylene,” group, it is understood that the range or number refers to the range or number of carbons in the linear divalent chain. “Heteroalkylene,” “heteroalkenylene,” and “heteroalkynylene” groups may be substituted or unsubstituted with one or more substituents as described herein.

“Aryl” refers to a radical of a monocyclic or polycyclic (e.g., bicyclic or tricyclic)₄n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl).

Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particular aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Arylene” as used herein, refers to an aryl group wherein two hydrogens are removed to provide a divalent radical. When a range or number of carbons is provided for a particular “arylene” group, it is understood that the range or number refers to the range or number of carbons in the aryl group. An “arylene” group may be substituted or unsubstituted with one or more substituents as described herein.

“Heteroaryl” refers to a radical of a 5- to 14-membered monocyclic or polycyclic 4n+2 aromatic ring system (e.g., having 6, 10, or 14 π electrons shared in a cyclic array) having ring carbon atoms and 1-8 ring heteroatoms provided in the aromatic ring system, wherein each heteroatom is independently selected from nitrogen, oxygen and sulfur (“5- to 14-membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings.

“Heteroaryl” also includes ring systems wherein the heteroaryl group, as defined above, is fused with one or more aryl groups wherein the point of attachment is either on the heteroaryl or the one or more aryl groups, and in such instances, the number of ring members designates the total number of ring members in the fused (aryl/heteroaryl) ring system. When substitution is indicated in such instances, unless otherwise specified, substitution can occur on either the heteroaryl or the one or more aryl groups. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In certain embodiments, a heteroaryl is a 5- to 10-membered aromatic ring system having ring carbon atoms and 1-4 ring heteroatoms provided in the aromatic ring system, wherein each heteroatom is independently selected from nitrogen, oxygen, and sulfur (“5- to 10-membered heteroaryl”). In certain embodiments, a heteroaryl is a 5- to 9-membered aromatic ring system having ring carbon atoms and 1-4 ring heteroatoms provided in the aromatic ring system, wherein each heteroatom is independently selected from nitrogen, oxygen, and sulfur (“5- to 9-membered heteroaryl”). In certain embodiments, a heteroaryl is a 5- to 8-membered aromatic ring system having ring carbon atoms and 1-4 ring heteroatoms provided in the aromatic ring system, wherein each heteroatom is independently selected from nitrogen, oxygen, and sulfur (“5- to 8-membered heteroaryl”). In certain embodiments, a heteroaryl group is a 5- to 6-membered aromatic ring system having ring carbon atoms and 1-4 ring heteroatoms provided in the aromatic ring system, wherein each heteroatom is independently selected from nitrogen, oxygen, and sulfur (“5- to 6-membered heteroaryl”). In certain embodiments, the 5- to 6-membered heteroaryl has 1-3 ring heteroatoms independently selected from nitrogen, oxygen, and sulfur. In certain embodiments, the 5- to 6-membered heteroaryl has 1-2 ring heteroatoms independently selected from nitrogen, oxygen, and sulfur. In certain embodiments, the 5- to 6-membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5- to 14-membered heteroaryl. In certain embodiments, the heteroaryl group is substituted 5- to 14-membered heteroaryl.

Exemplary 5-membered heteroaryl containing one heteroatom include, without limitation, pyrrolyl, furanyl and thiophenyl. Exemplary 5-membered heteroaryl containing two heteroatoms include, without limitation, imidazolyl, pyrazolyl, oxazolyl, isoxazolyl, thiazolyl, and isothiazolyl. Exemplary 5-membered heteroaryl containing three heteroatoms include, without limitation, triazolyl, oxadiazolyl, and thiadiazolyl. Exemplary 5-membered heteroaryl containing four heteroatoms include, without limitation, tetrazolyl. Exemplary 6-membered heteroaryl containing one heteroatom include, without limitation, pyridinyl. Exemplary 6-membered heteroaryl containing two heteroatoms include, without limitation, pyridazinyl, pyrimidinyl, and pyrazinyl. Exemplary 6-membered heteroaryl containing three or four heteroatoms include, without limitation, triazinyl and tetrazinyl, respectively. Exemplary 7-membered heteroaryl containing one heteroatom include, without limitation, azepinyl, oxepinyl, and thiepinyl. Exemplary 5,6-bicyclic heteroaryl include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl include, without limitation, naphthyridinyl, pteridinyl, quinolinyl, isoquinolinyl, cinnolinyl, quinoxalinyl, phthalazinyl, and quinazolinyl.

“Heteroarylene” as used herein, refers to a heteroaryl group wherein two hydrogens are removed to provide a divalent radical. When a range or number of ring members is provided for a particular “heteroarylene” group, it is understood that the range or number refers to the number of ring members in the heteroaryl group. A “heteroarylene” group may be substituted or unsubstituted with one or more substituents as described herein.

“Carbocyclyl” refers to a radical of a non-aromatic cyclic hydrocarbon group having from 3 to 12 ring carbon atoms (“C₃₋₁₂ carbocyclyl”) and zero heteroatoms in the nonaromatic ring system. In certain embodiments, a carbocyclyl group has 3 to 10 ring carbon atoms (“C₃₋₁₀ carbocyclyl”). In certain embodiments, a carbocyclyl group has 3 to 8 ring carbon atoms (“C₃₋₈ carbocyclyl”). In certain embodiments, a carbocyclyl group has 3 to 6 ring carbon atoms (“C₃₋₆ carbocyclyl”). In certain embodiments, a carbocyclyl group has 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, a carbocyclyl group has 5 or 6 ring carbon atoms (“C₅₋₆ carbocyclyl”). Exemplary C₃₋₆ carbocyclyl include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₅), and the like. Exemplary C₃₋₁₀ carbocyclyl include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

In certain embodiments, “carbocyclyl” is a monocyclic, saturated carbocyclyl group having from 3 to 12 ring carbon atoms (“C₃₋₁₂ carbocyclyl”). In certain embodiments, “carbocyclyl” is a monocyclic, saturated carbocyclyl group having from 3 to 10 ring carbon atoms (“C₃₋₁₀ carbocyclyl”). In certain embodiments, “carbocyclyl” is a monocyclic, saturated carbocyclyl group having from 3 to 8 ring carbon atoms (“C₃₋₈ carbocyclyl”). In certain embodiments, “carbocyclyl” is a monocyclic, saturated carbocyclyl group having from 3 to 6 ring carbon atoms (“C₃₋₆ carbocyclyl”). In certain embodiments, “carbocyclyl” is a monocyclic, saturated carbocyclyl group having from 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, “carbocyclyl” is a monocyclic, saturated carbocyclyl group having 5 or 6 ring carbon atoms (“C₅₋₆ carbocyclyl”). Examples of C₅₋₆ carbocyclyl include cyclopentyl (C₅) and cyclohexyl (C₅). Examples of C₃₋₆ carbocyclyl include the aforementioned C₅₋₆ carbocyclyl groups as well as cyclopropyl (C₃) and cyclobutyl (C₄). Examples of C₃₋₈ carbocyclyl include the aforementioned C₃₋₆ carbocyclyl groups as well as cycloheptyl (C₇) and cyclooctyl (C₈). Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is substituted C₃₋₁₂ carbocyclyl.

As the foregoing examples illustrate, in certain embodiments, the carbocyclyl group is either monocyclic (“monocyclic carbocyclyl”) or polycyclic (“polycyclic carbocyclyl”) that contains a fused, bridged or spiro ring system and can be saturated or can be partially unsaturated. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is a substituted C₃₋₁₂ carbocyclyl.

“Fused carbocyclyl” or “fused carbocycle” refers to ring systems wherein the carbocyclyl group, as defined above, is fused with, i.e., share two common atoms (as such, share one common bond), one or more carbocyclyl groups, as defined above, wherein the point of attachment is on any of the fused rings. In such instances, the number of carbons designates the total number of carbons in the fused ring system. When substitution is indicated, unless otherwise specified, substitution can occur on any of the fused rings.

“Spiro carbocyclyl” or “spiro carbocycle” refers to ring systems wherein the carbocyclyl group, as defined above, form spiro structure with, i.e., share one common atom with, one or more carbocyclyl groups, as defined above, wherein the point of attachment is on the carbocyclyl rings in which the spiro structure is embedded. In such instances, the number of carbons designates the total number of carbons of the carbocyclyl rings in which the spiro structure is embedded. When substitution is indicated, unless otherwise specified, substitution can occur on the carbocyclyl rings in which the spiro structure is embedded.

“Bridged carbocyclyl” or or “bridged carbocycle” refers to ring systems wherein the carbocyclyl group, as defined above, form bridged structure with, i.e., share more than two atoms (as such, share more than one bonds) with, one or more carbocyclyl groups, as defined above, wherein the point of attachment is on any of the carbocyclyl rings in which the bridged structure is embedded. In such instances, the number of carbons designates the total number of carbons of the carbocyclyl rings in which the bridged structure is embedded. When substitution is indicated, unless otherwise specified, substitution can occur on any of the carbocyclyl rings in which the bridged structure is embedded.

“Carbocyclylene” as used herein, refers to a carbocyclyl group wherein two hydrogens are removed to provide a divalent radical. The divalent radical may be present on different atoms or the same atom of the carbocyclylene group. When a range or number of carbons is provided for a particular “carbocyclyl” group, it is understood that the range or number refers to the range or number of carbons in the carbocyclyl group. A “carbocyclyl” group may be substituted or unsubstituted with one or more substituents as described herein.

“Heterocyclyl” refers to a radical of a 3- to 12-membered non-aromatic ring system having ring carbon atoms and 1 to 4 ring heteroatoms, wherein each heteroatom is independently selected from nitrogen, oxygen, sulfur, boron, phosphorus, and silicon (“3- to 12-membered heterocyclyl”). In heterocyclyl groups that contain one or more nitrogen atoms, the point of attachment can be a carbon or nitrogen atom, as valency permits. Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

In certain embodiments, a heterocyclyl group is a 5- to 12-membered non-aromatic ring system having ring carbon atoms and 1-4 ring heteroatoms, wherein each heteroatom is independently selected from nitrogen, oxygen, sulfur, boron, phosphorus, and silicon (“5- to 12-membered heterocyclyl”). In certain embodiments, a heterocyclyl group is a 5- to 10-membered non-aromatic ring system having ring carbon atoms and 1-4 ring heteroatoms, wherein each heteroatom is independently selected from nitrogen, oxygen, sulfur, boron, phosphorus, and silicon (“5- to 10-membered heterocyclyl”). In certain embodiments, a heterocyclyl group is a 5- to 8-membered non-aromatic ring system having ring carbon atoms and 1-4 ring heteroatoms, wherein each heteroatom is independently selected from nitrogen, oxygen, and sulfur (“5- to 8-membered heterocyclyl”). In certain embodiments, a heterocyclyl group is a 5- to 6-membered non-aromatic ring system having ring carbon atoms and 1-4 ring heteroatoms, wherein each heteroatom is independently selected from nitrogen, oxygen, and sulfur (“5- to 6-membered heterocyclyl”). In certain embodiments, the 5- to 6-membered heterocyclyl has 1-3 ring heteroatoms selected from nitrogen, oxygen, and sulfur. In certain embodiments, the 5- to 6-membered heterocyclyl has 1-2 ring heteroatoms selected from nitrogen, oxygen, and sulfur. In certain embodiments, the 5- to 6-membered heterocyclyl has one ring heteroatom selected from nitrogen, oxygen, and sulfur.

As the foregoing examples illustrate, in certain embodiments, a heterocyclyl group can either be monocyclic (“monocyclic heterocyclyl”) or polycyclic (“polycyclic heterocyclyl”) that contains a fused, bridged or spiro ring system, and can be saturated or can be partially unsaturated. Heterocyclyl polycyclic ring systems can include one or more heteroatoms in one or both rings. “Heterocyclyl” also includes ring systems wherein the heterocyclyl group, as defined above, is fused with one or more carbocyclyl groups wherein the point of attachment is either on the carbocyclyl or heterocyclyl ring, and in such instances, the number of ring members designates the total number of ring members in the entire ring system. When substitution is indicated in such instances, unless otherwise specified, substitution can occur on either the heterocyclyl or the one or more carbocyclyl groups. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3- to 12-membered heterocyclyl. In certain embodiments, the heterocyclyl group is substituted 3- to 12-membered heterocyclyl.

“Fused heterocyclyl” or “fused heterocycle” refers to ring systems wherein the heterocyclyl group, as defined above, is fused with, i.e., share two common atoms (as such, share one common bond) with, one or more heterocyclyl or carbocyclyl groups, as defined above, wherein the point of attachment is on any of the fused rings. In such instances, the number of ring members designates the total number of ring members in the fused ring system. When substitution is indicated, unless otherwise specified, substitution can occur on any of the fused rings.

“Spiro heterocyclyl” or “spiro heterocycle” refers to ring systems wherein the heterocyclyl group, as defined above, form spiro structure with, i.e., share one common atom with, one or more heterocyclyl or carbocyclyl groups, as defined above, wherein the point of attachment is on the heterocyclyl or carbocyclyl rings in which the spiro structure is embedded. In such instances, the number of ring members designates the total number of ring members of the heterocyclyl or carbocyclyl rings in which the spiro structure is embedded. When substitution is indicated, unless otherwise specified, substitution can occur on any of the heterocyclyl or carbocyclyl rings in which the spiro structure is embedded.

“Bridged heterocyclyl” or “bridged heterocycle” refers to ring systems wherein the heterocyclyl group, as defined above, form bridged structure with, i.e., share more than two atoms (as such, share more than one bonds) with, one or more heterocyclyl or carbocyclyl groups, as defined above, wherein the point of attachment is on the heterocyclyl or carbocyclyl rings in which the bridged structure is embedded. In such instances, the number of ring members designates the total number of ring members of the heterocyclyl or carbocyclyl rings in which the bridged structure is embedded. When substitution is indicated, unless otherwise specified, substitution can occur on any of the heterocyclyl or carbocyclyl rings in which the bridged structure is embedded.

“Heterocyclylene” as used herein, refers to a heterocyclyl group wherein two hydrogens are removed to provide a divalent radical. The divalent radical may be present on different atoms or the same atom of the heterocyclylene group. When a range or number of ring members is provided for a particular “heterocyclylene” group, it is understood that the range or number refers to the number of ring members in the heterocyclylene group. A “heterocyclylene” group may be substituted or unsubstituted with one or more substituents as described herein.

“Alkoxy” as used herein, refers to the group —OR, wherein R is alkyl as defined herein. C₁₋₆ alkoxy refers to the group —OR, wherein each R is C₁₋₆ alkyl, C₃₋₄ carbocycyl, or 3- to 4-membered heterocyclyl, as defined herein. Exemplary C₁₋₆ alkyl is set forth above.

“Alkylamino” as used herein, refers to the group —NHR or —NR₂, wherein each R is independently alkyl, as defined herein. C₁₋₆ alkylamino refers to the group —NHR or —NR₂, wherein each R is independently C₁₋₆ alkyl, C₃₋₄ carbocycyl, or 3- to 4-membered heterocyclyl, as defined herein. Exemplary C₁₋₆ alkyl is set forth above.

“Oxo” refers to ═O. When a group other than aryl and heteroaryl or an atom is substituted with an oxo, it is meant to indicate that two geminal radicals on that group or atom form a double bond with an oxygen radical. When a heteroaryl is substituted with an oxo, it is meant to indicate that a resonance structure/tautomer involving a heteroatom provides a carbon atom that is able to form two geminal radicals, which form a double bond with an oxygen radical.

“Halo” or “halogen” refers to fluoro (F), chloro (CI), bromo (Br), and iodo (I). In certain embodiments, the halo group is either fluoro or chloro.

“Protecting group” as used herein is art-recognized and refers to a chemical moiety introduced into a molecule by chemical modification of a functional group (e.g., hydroxyl, amino, thio, and carboxylic acid) to obtain chemoselectivity in a subsequent chemical reaction, during which the unmodified functional group may not survive or may interfere with the chemical reaction. Common functional groups that need to be protected include but not limited to hydroxyl, amino, thiol, and carboxylic acid. Accordingly, the protecting groups are termed hydroxyl-protecting groups, amino-protecting groups, thiol-protecting groups, and carboxylic acid-protecting groups, respectively.

Common types of hydroxyl-protecting groups include but not limited to ethers (e.g., methoxymethyl (MOM), β-Methoxyethoxymethyl (MEM), tetrahydropyranyl (THP), p-methoxyphenyl (PMP), t-butyl, triphenylmethyl (Trityl), allyl, and benzyl ether (Bn)), silyl ethers (e.g., t-butyldiphenylsilyl (TBDPS), trimethylsilyl (TMS), triisopropylsilyl (TIPS), tri-iso-propylsilyloxymethyl (TOM), and t-butyldimethylsilyl (TBDMS)), and esters (e.g., pivalic acid ester (Piv) and benzoic acid ester (benzoate; Bz)).

Common types of amino-protecting groups include but not limited to carbamates (e.g., t-butyloxycarbonyl (Boc), 9-fluorenylmethyloxycarbonyl (Fmoc), p-methoxybenzyl carbonyl (Moz or MeOZ), 2,2,2-trichloroehtoxycarbonyl (Troc), and benzyl carbamate (Cbz)), esters (e.g., acetyl (Ac); benzoyl (Bz), trifluoroacetyl, and phthalimide), amines (e.g, benzyl (Bn), p-methoxybenzyl (PMB), p-methoxyphenyl (PMP), and triphenylmethyl (trityl)), and sulfonamides (e.g., tosyl (Ts), N-alkyl nitrobenzenesulfonamides (Nosyl), and 2-nitrophenylsulfenyl (Nps)).

Common types of thiol-protecting groups include but not limited to sulfide (e.g., p-methylbenzyl (Meb), t-butyl, acetamidomethyl (Acm), and triphenylmethyl (Trityl)).

Common types of carboxylic acid-protecting groups include but not limited to esters (e.g., methyl ester, triphenylmethyl (Trityl), t-butyl ester, benzyl ester (Bn), S-f-butyl ester, silyl esters, and orthoesters) and oxazoline.

These and other exemplary substituents are described in more detail in the Detailed Description, Examples, and claims. The invention is not intended to be limited in any manner by the above exemplary listing of substituents.

### Other Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo [2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

The term “pharmaceutically acceptable cation” refers to an acceptable cationic counterion of an acidic functional group. Such cations are exemplified by sodium, potassium, calcium, magnesium, ammonium, tetraalkylammonium cations, and the like (see, e.g., Berge, et al., J. Pharm. Sci. 66 (1): 1-79 (January 77).

“Pharmaceutically acceptable vehicle” refers to a diluent, adjuvant, excipient or carrier with which a compound of the invention is administered.

“Pharmaceutically acceptable metabolically cleavable group” refers to a group which is cleaved in vivo to yield the parent molecule of the structural formula indicated herein. Examples of metabolically cleavable groups include —COR, —COOR, —CONR₂ and —CH₂OR radicals, where R is selected independently at each occurrence from alkyl, trialkylsilyl, carbocyclic aryl or carbocyclic aryl substituted with one or more of alkyl, halogen, hydroxy or alkoxy. Specific examples of representative metabolically cleavable groups include acetyl, methoxycarbonyl, benzoyl, methoxymethyl and trimethylsilyl groups.

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the invention may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

A “subject” to which administration is contemplated includes, but is not limited to, humans (i.e., a male or female of any age group, e.g., a pediatric subject (e.g, infant, child, adolescent) or an adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

An “effective amount” means the amount of a compound that, when administered to a subject for treating or preventing a disease, is sufficient to effect such treatment or prevention. The “effective amount” can vary depending on the compound, the disease and its severity, and the age, weight, etc., of the subject to be treated. A “therapeutically effective amount” refers to the effective amount for therapeutic treatment. A “prophylatically effective amount” refers to the effective amount for prophylactic treatment.

“Preventing”, “prevention” or “prophylactic treatment” refers to a reduction in risk of acquiring or developing a disease or disorder (i.e., causing at least one of the clinical symptoms of the disease not to develop in a subject not yet exposed to a disease-causing agent, or in a subject who is predisposed to the disease in advance of disease onset).

The term “prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment, “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of its electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure (R)-compound” refers to at least about 95% by weight (R)-compound and at most about 5% by weight(S)-compound, at least about 99% by weight (R)-compound and at most about 1% by weight(S)-compound, or at least about 99.9% by weight (R)-compound and at most about 0.1% by weight(S)-compound. In certain embodiments, the weights are based upon total weight of compound.

As used herein and unless otherwise indicated, the term “enantiomerically pure(S)-compound” or “(S)-compound” refers to at least about 95% by weight(S)-compound and at most about 5% by weight (R)-compound, at least about 99% by weight(S)-compound and at most about 1% by weight (R)-compound or at least about 99.9% by weight(S)-compound and at most about 0.1% by weight (R)-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure (R)-compound can comprise, for example, about 90% excipient and about 10% enantiomerically pure (R)-compound. In certain embodiments, the enantiomerically pure (R)-compound in such compositions can, for example, comprise, at least about 95% by weight (R)-compound and at most about 5% by weight(S)-compound, by total weight of the compound. For example, a pharmaceutical composition comprising enantiomerically pure(S)-compound can comprise, for example, about 90% excipient and about 10% enantiomerically pure(S)-compound. In certain embodiments, the enantiomerically pure(S)-compound in such compositions can, for example, comprise, at least about 95% by weight(S)-compound and at most about 5% by weight (R)-compound, by total weight of the compound. In certain embodiments, the active ingredient can be formulated with little or no excipient or carrier.

The compounds of this invention may possess one or more asymmetric centers; such compounds can therefore be produced as individual (R)- or(S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

The term “about” when referring to a number or a numerical range means that the number or numerical range referred to is an approximation within experimental variability (or within statistical experimental error), and thus the number or numerical range, in some instances, will vary between 1% and 15% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### I. Synthesis and Characterization of Intermediates and Compounds 1-318

In the following examples, the chemical reagents were purchased from commercial sources (such as Alfa, Acros, Sigma Aldrich, TCI and Shanghai Chemical Reagent Company), and used without further purification.

In obtaining the compounds described in the examples below and the corresponding analytical data, the following experimental and analytical protocols were followed unless otherwise indicated.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Compound purification was carried out as needed using a variety of traditional methods including, but not limited to, preparative chromatography under acidic, neutral, or basic conditions using either normal phase or reverse phase HPLC or flash columns or Prep-TLC plates.

Flash chromatography was performed on a Biotage Isolera One via column with silica gel particles of 200-300 mesh. Analytical and preparative thin-layer chromatography was performed using silica gel 60 GF254 plates. Normal-phase silica gel chromatography (FCC) was also performed on silica gel (SiO₂) using prepacked cartridges.

Preparative reverse-phase high performance liquid chromatography (RP HPLC) was performed on either:

### Method A

Prep-HPLC with YMC-Actus Triart 18C(5 μm, 20×250 mm), and mobile phase of 5-99% ACN in water (0.1% HCOOH) over 10 min and then hold at 100% ACN for 2 min, at a flow rate of 25 mL/min; or

### Method B

Preparative supercritical fluid high performance liquid chromatography (SFC) was performed either on a Thar 80 Prep-SFC system, or Waters 80Q Prep-SFC system from Waters. The ABPR was set to 100bar to keep the CO₂ in SF conditions, and the flow rate may verify according to the compound characteristics, with a flow rate ranging from 50g/min to 70g/min. The column temperature was ambient temperature.

Nuclear magnetic resonance (NMR) spectra were recorded using Brucker AVANCE NEO 400 MHz at around 20-30° C. unless otherwise specified. The following abbreviations are used: s, singlet; d, doublet; t, triplet; q, quartet; m, multiplet; dd, doublet of doublets; ddd, doublet of doublet of doublet; dt, doublet of triplets; bs, broad signal. Chemical shifts were reported in parts per million (ppm, 8) downfield from tetramethylsilane. It will be understood that for compounds comprising an exchangeable proton, said proton may or may not be visible on an NMR spectrum depending on the choice of solvent used for running the NMR spectrum and the concentration of the compound in the solution.

Mass spectra (MS) were obtained on a SHIMADZU LCMS-2020 MSD using electrospray ionization (ESI) in positive mode unless otherwise indicated. Calculated (calcd.) mass corresponds to the exact mass.

Chemical names were generated using ChemDraw Ultra 12.0, ChemDraw Ultra 14.0 (CambridgeSoft Corp., Cambridge, MA) or ACD/Name Version 10.01 (Advanced Chemistry).

Compounds designated as R*or S*are enantiopure compounds where the absolute configuration was not determined.

### Intermediate 1:5-chloroimidazo[1,5-a]pyridine-7-carbaldehyde

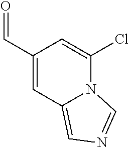 [SMILES: O=Cc1cc(Cl)n2cncc2c1]

### Step A: methyl 2-(bromomethyl)-6-chloropyridine-4-carboxylate

A solution of methyl 2-chloro-6-methylpyridine-4-carboxylate (9 g, 48.489 mmol, 1.0 eq), NBS (11.22 g, 63.036 mmol, 1.3 eq), and AIBN (0.717 mL, 4.849 mmol, 0.1 eq) in CCl₄ (180 mL) was stirring under nitrogen at 90° C. for 12 h. After cooled to room temperature, the mixture was diluted with cold water (300 mL) and extracted with DCM (300 mL×3). The combined organic extracts were washed with water (200 mL), dried over anhydrous sodium sulfate, filtered and concentrated under reduced pressure. The residue was purified by flash column chromatography on silica gel (EA in PE, from 0 to 10%) to give a mixture (20 g). To the solution of mixture (20 g) in THF (300 mL) was added DIPEA (25.3 mL, 145.467 mmol, 3.0 eq) and Diethyl phosphite (18.7 mL, 145.467 mmol, 3.0 eq) at 0° C. and the resulting mixture was stirred at room temperature for 4 h. The mixture was diluted with (300 mL) and extracted with EA (300 mL×3). The organic layer was washed with water (300 mL×3), dried over anhydrous sodium sulfate, filtered and concentrated under reduced pressure. The residue was purifed by flash column chromatography on silica gel (EA in PE, from 0 to 4%) to give methyl 2-(bromomethyl)-6-chloroisonicotinate (10 g, yield 78%) as a white solid.

LC-MS (ESI): mass calcd. for C₈H₇BrClNO₂, 262.93; m/z found, 264.3 [M+H]⁺.

### Step B: methyl 2-(azidomethyl)-6-chloropyridine-4-carboxylate

A solution of methyl 2-(bromomethyl)-6-chloropyridine-4-carboxylate (8 g, 30.245 mmol, 1.0 eq) and sodium azide (3.93 g, 60.489 mmol, 2.0 eq) in DMF (80 mL) was stirring for 12 h at room temperature. The mixture was diluted with cold water (200 mL) and extracted with ethyl acetate (200 mL×3). The combined organic extracts were washed with brine (150 mL×4), dried over anhydrous sodium sulfate, filtered and concentrated under reduced pressure to provide crude product methyl 2-(azidomethyl)-6-chloropyridine-4-carboxylate (6 g, yield 87%) as a yellow oil.

LC-MS (ESI): mass calcd. for C₈H₇ClN₄O₂, 226.03; m/z found, 227.1 [M+H]⁺.

### Step C: methyl 2-(aminomethyl)-6-chloropyridine-4-carboxylate

A solution of methyl 2-(azidomethyl)-6-chloropyridine-4-carboxylate (6 g, 26.476 mmol, 1.0 eq) and PPh3 (10.42 g, 39.714 mmol, 1.5 eq) in THF (80 mL) and H₂O (8 mL) was heated at 50° C. for 1 h. After evaporation, the mixture was dissolved in aqueous HCl solution (2 N) (50 mL) and extracted with DCM (50 mL×3). The organic layers were discarded off and the aqueous layer was concentrated under reduced pressure to obtain methyl 2-(aminomethyl)-6-chloropyridine-4-carboxylate hydrochloride (4.4 g, yield 70%) as a white solid.

LC-MS (ESI): mass calcd. for C₈H₉ClN₂O₂, 200.04; m/z found, 201.1 [M+H]⁺.

### Step D: methyl 2-chloro-6-(formamidomethyl)pyridine-4-carboxylate

To a solution of methyl 2-(aminomethyl)-6-chloropyridine-4-carboxylate hydrochloride (1.5 g, 7.476 mmol, 1.0 eq) in ethyl formate (30 mL) was added NaHCO₃ (0.31 g, 3.738 mmol, 0.5 eq) and triethylamine (1.6 mL, 11.214 mmol, 1.5 eq) and the mixture was refluxed for 10 h. After filtration, the filtrate was concentrated to gave crude product methyl 2-chloro-6-(formamidomethyl)pyridine-4-carboxylate (1.20 g, yield 70%) as a brown oil. The crude product was used in next step without further purification.

LC-MS (ESI): mass calcd. for C₉H₉ClN₂O₃, 228.03; m/z found, 229.2 [M+H]⁺.

### Step E: methyl 5-chloroimidazo[1,5-a]pyridine-7-carboxylate

To a solution of methyl 2-chloro-6-(formamidomethyl)pyridine-4-carboxylate (1.2 g, 5.249 mmol, 1.0 eq) in dioxane (20 mL) was added POCl₃ (0.978 mL, 10.497 mmol, 2.0 eq) and the mixture was refluxed for 3 hours. The reaction mixture was cooled to room temperature, quenched with saturated aqueous NaHCO₃ solution (50 mL) at 0° C. and extracted with EtOAc (30 mL×3). The organic layers were washed with brine (30 mL), dried over anhydrous Na₂SO₄, filtered and concentrated under reduced pressure. The crude product was purified by flash column chromatography on silica gel (EtOAc in petroleum ether, 30%) to give methyl 5-chloroimidazo[1,5-a]pyridine-7-carboxylate (340 mg, yield 28%) as a yellow solid.

LC-MS (ESI): mass calcd. for C₉H₇ClN₂O₂, 210.02; m/z found, 211.2 [M+H]⁺. ¹H NMR (400 MHZ, DMSO-d₆): δ 8.69 (s, 1H), 8.41 (s, 1H), 7.94 (s, 1H), 7.25 (s, 1H), 3.87 (s, 3H).

### Step F: {5-chloroimidazo[1,5-a]pyridin-7-yl}methanol

To a solution of methyl 5-chloroimidazo[1,5-a]pyridine-7-carboxylate (340 mg, 1.614 mmol, 1.0 eq) in dry THF (15 mL) was added dropwise DIBAL-H (1 Nin THF) (2.4 mL, 2.421 mmol, 1.5 eq) under N₂ at −78 oC and the mixture was stirred at room temperature for 30 min. The mixture was diluted with THF (30 mL) and slowly quenched with Na₂SO₄·10H₂O, and filtered. The filtrate was concentrated and purified by column chromatography on silica gel (EtOAc in petroleum ether, 50%) to give {5-chloroimidazo[1,5-a]pyridin-7-yl}methanol (250 mg, yield 76.33%) as a yellow solid.

LC-MS (ESI): mass calcd. for C₈H₇ClN₂O, 182.02; m/z found, 183.2 [M+H]⁺.

### Step G: 5-chloroimidazo[1,5-a]pyridine-7-carbaldehyde

To a solution of {5-chloroimidazo[1,5-a]pyridin-7-yl}methanol (250 mg, 1.369 mmol, 1.0 eq) in DCM (5 mL) was added Dess-Martin periodinane (1.279 mL, 4.107 mmol, 3.0 eq) and the reaction was stirring at room temperature for 1 h. The reaction mixture was quenched with aqueous Na₂SO₃ (20 mL) and extracted with EtOAc (30 mL×3). The organic layers were washed with brine (20 mL), dried over anhydrous Na₂SO₄, filtered and concentrated under reduced pressure. The crude product was purified by flash column chromatography on silica gel (EtOAc in petroleum ether, 50%) to give 5-chloroimidazo[1,5-alpyridine-7-carbaldehyde (200 mg, yield 73%) as a yellow solid.

LC-MS (ESI): mass calcd. for C₈H₅ClN₂O, 180.01; m/z found, 181.2 [M+H]⁺. ¹H NMR (400 MHZ, DMSO-d₆): δ9.82 (s, 1H), 8.71 (s, 1H), 8.43 (s, 1H), 8.05 (s, 1H), 7.15 (d, J=1.0 hz, 1H).

### Intermediate 2:8-bromoimidazo[1,2-a]pyridine-6-carbaldehyde

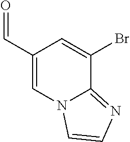 [SMILES: O=Cc1cc(Br)c2nccn2c1]

### Step A: methyl 8-bromoimidazo[1,2-a]pyridine-6-carboxylate

A solution of methyl 6-amino-5-bromopyridine-3-carboxylate (2 g, 8.656 mmol, 1.0 eq), 2-chloroacetaldehyde (2.062 mL, 12.984 mmol, 1.5 eq), and NaHCO₃ (1.09 g, 12.984 mmol, 1.5 eq) in EtOH (40 mL) was stirred at 80° C. for 12 h under nitrogen. After evaporation, the mixture was diluted with cold water (100 mL) and extracted with ethyl acetate (60 mL×3). The combined organic extracts were washed with water (100 mL), dried over anhydrous sodium sulfate, filtered and concentrated under reduced pressure. The residue was purified by flash column chromatography on silica gel (EA/PE=1/2) to obtain methyl 8-bromoimidazo[1,2-a]pyridine-6-carboxylate (1.5 g, yield 61%) as a yellow solid.

LC-MS (ESI): mass calcd. for C₉H₇BrN₂O₂, 253.97; m/z found, 254.4 [M+H]⁺. ¹HNMR (400 MHZ, CDCl₃) δ 8.90 (s, 1H), 8.01 (s, 1H), 7.76 (d, J=6.8 Hz, 2H), 3.96 (s, 3H).

### Step B: {8-bromoimidazo[1,2-a]pyridin-6-yl}methanol

To a solution of methyl 8-bromoimidazo[1,2-a]pyridine-6-carboxylate (15 g, 58.807 mmol, 1.0 eq) in THF (300 mL) was added dropwise DIBAL-H (1 N in THF) (88.2 mL, 88.210 mmol, 1.5 eq) under N₂ at −78° C. Then the mixture was warmed to 0° C. and stirred for 1 hours. The reaction mixture was diluted with THF (150 mL), slowly quenched with Na₂SO₄·10H₂O. After filtration, The fitrate was concentrated and purified by flash column chromatography on silica gel (EA/PE=1/1) to obtain {8-bromoimidazo[1,2-a]pyridin-6-yl}methanol (10 g, yield 67.4%) as a white solid.

LC-MS (ESI): mass calcd. for C₈H₇BrN₂O, 225.97; m/z found, 227.4 [M+H]⁺.

### Step C: 8-bromoimidazo[1,2-a]pyridine-6-carbaldehyde

To a solution of {8-bromoimidazo[1,2-a]pyridin-6-yl}methanol (10 g, 44.041 mmol, 1.0 eq) in DCM (100 mL) was added Dess-Martin periodinane (41.145 mL, 132.124 mmol, 3.0 eq) and the mixture stirring at room temperature for 1 hours. The residue was poured into water (60 mL) and extracted with EtOAc (60 mL×4). The organic layer was dried over anhydrous MgSO₄, filtered and concentrated under reduced pressure. The residue was purified by flash column chromatography on silica gel (EA/PE=1/1) to obtain 8-bromoimidazo[1,2-a]pyridine-6-carbaldehyde (8 g, yield 73%) as a yellow solid.

LC-MS (ESI): mass calcd. for C₈HsBrN₂O, 223.96; m/z found, 225.5 [M+H]⁺

### Intermediate 3:5-chloro-[1,2,4]triazolo[4,3-a]pyridine-7-carbaldehyde

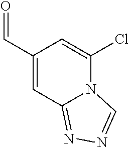 [SMILES: O=Cc1cc(Cl)n2cnnc2c1]

### Step A: tert-butyl 2-chloro-6-hydrazinylpyridine-4-carboxylate

To a solution of tert-butyl 2,6-dichloropyridine-4-carboxylate (3.912 mL, 20.152 mmol, 1.0 eq) in EtOH (20 mL) was added Hydrazine (3.23 g, 100.762 mmol, 5.0 eq). The mixture was stirred at 75° C. for 18 h. The mixture was cooled to room temperature, concentrate under reduced pressure to half of the volume, and solid precipitated. The solid was filtered off and the filtrate was concentrated to dryness to give crude tert-butyl 2-chloro-6-hydrazinylpyridine-4-carboxylate (4.1 g, yield 83%) as a yellow solid.

LC-MS (ESI): mass calced for: C₁₀H₁₄ClN₃O₂ 243.08; m/z found, 244.0 [M+H]⁺.

### Step B: tert-butyl 7-chloro-[1,2,4]triazolo[4,3-a]pyridine-5-carboxylate

A mixture of tert-butyl 2-chloro-6-hydrazinylpyridine-4-carboxylate (4.1 g, 16.825 mmol, 1.0 eq) in trimethyl orthoformate (15 mL) was stirred at 85° C. for 5 h. The mixture was cooled to room temperature and concentrated under reduced pressure. The residue was purified by flash chromatography on silica gel (ethyl acetate in petroleum ether, from 0 to 50%) to provide tert-butyl 7-chloro-[1,2,4]triazolo[4,3-a]pyridine-5-carboxylate (1.8 g, yield 42%).

LC-MS (ESI): mass calced for: C₁₁H₁₂ClN₃O₂ 253.06; m/z found, 254.1 [M+H]⁺. ¹H NMR (400 MHZ, DMSO-d₆) δ 9.61 (s, 1H), 8.33 (s, 1H), 7.47 (d, J=1.0 Hz, 1H), 1.59 (s, 9H).

### Step C: 7-chloro-[1,2,4]triazolo[4,3-a]pyridine-5-carboxylic acid

TFA (20 mL) was added to a solution of tert-butyl 7-chloro-[1,2,4]triazolo[4,3-alpyridine-5-carboxylate (4.2 g, 16.556 mmol, 1.0 eq) in DCM (20 mL). The reaction mixture was stirred at room temperature for 4 h. The mxiture was concentrated under reduced pressure to give 7-chloro-[1,2,4]triazolo[4,3-a]pyridine-5-carboxylic acid (1.8 g, yield 55%) as a crude yellow solid.

LC-MS (ESI): mass calced for: C₇H₄ClN₃O₂ 197.00; m/z found, 198.1 [M+H]⁺.

### Step D: (5-chloro-[1,2,4]triazolo[4,3-a]pyridin-7-yl) methanol

To a solution of 7-chloro-[1,2,4]triazolo[4,3-a]pyridine-5-carboxylic acid (1.8 g, 9.110 mmol, 1.0 eq) was THF (20 mL) was added dropwise Borane-tetrahydrofuran complex (1 N) (45.5 mL, 45.551 mmol, 5.0 eq) at 0° C. The reaction mixture was warmed at room temperature for 24 h. After cooled to 0° C., the mixture was slowly quenched with methanol (50 mL) and refluxed for 1 h. After evaporation, the residue was partitioned with ethyl acetate (200 mL) and aqueous NaOH solution (50 mL). The organic layer was washed with brine (50 mL), dried over Na₂SO₄, filtered and concentrated under reduced pressure. The residue was purified with flash column chromatography on silica gel (methanol in chloroform, from 0 to 5%) to give {7-chloro-[1,2,4]triazolo[4,3-a]pyridin-5-yl}methanol (760 mg, yield 45%) as a yellow solid.

LC-MS (ESI): mass calced for: C₇H₆ClN30 183.02; m/z found, 184.1 [M+H]⁺.

### Step E: 7-chloro-[1,2,4]triazolo[4,3-a]pyridine-5-carbaldehyde

To a solution of {7-chloro-[1,2,4]triazolo[4,3-a]pyridin-5-yl}methanol (620 mg, 3.377 mmol, 1.0 eq) in DCM (20 mL) was added Dess-Martin Periodinane (2.15 g, 5.065 mmol, 1.5 eq) and the mixture was stirred at room temperature for 2 h. The mixturewas diluted with EtOAc (60 mL), washed with saturated aqueous Na₂S₂O₃ solution (30 mL), saturated aqueous NaHCO₃ solution (20 mL) and brine (20 mL). The organic layer was dried over anhydrous Na₂SO₄, filtered and concentrated under reduced pressure. The crude product was purified by flash columnchromatography on silica gel (Petroleum ether/EtOAc=50/1) to afford 7-chloro-[1,2,4]triazolo[4,3-a]pyridine-5-carbaldehyde (350 mg, yield 57%) as a yellow oil. LC-MS (ESI): mass calced for: C₇H₄ClN30 181.02; m/z found, 182.1 [M+H]⁺.

### Intermediate 4:4-bromo-1-methyl-1H-benzo[d]imidazole-6-carbaldehyde

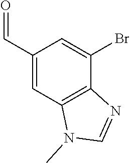

### Step A: 4-bromo-1-methyl-1H-1,3-benzodiazole-6-carbonitrile

To a solution of 4-bromo-1H-1,3-benzodiazole-6-carbonitrile (1.0 g, 4.504 mmol, 1.0 eq) in DMF (20 mL) was added NaH (60% suspend in oil) (0.27 g, 6.756 mmol, 1.5 eq) at 0° C. under nitrogen. The reaction mixture was stirred at 0° C. for 1 hr, then iodomethane (0.83 g, 5.855 mmol, 1.3 eq) was dropwised to above mixture and the mixture was stirred at 0° C. for 1 h. The mixture was diluted with cold water (40 mL) and extracted with ethyl acetate (30 mL×3). The combined organic extracts were washed with brine (30 mL×4), dried over sodium sulfate, filtered and concentrated under reduced pressure. The residue was purified by flash column chromatography on silica gel (ethyl acetate in petroleum ether, 10%) to get 4-bromo-1-methyl-1H-benzo[d]imidazole-6-carbonitrile (0.314 g, yield 29%) as a white solid.

LC-MS (ESI): mass calcd. for C₉H₆BrN3, 234.97; m/z found, 235.98 [M+H]⁺. ¹H NMR (400 MHZ, DMSO-d₆) δ 8.56 (s, 1H), 8.31 (d, J=1.2 Hz, 1H), 7.92 (d, J=1.2 Hz, 1H), 3.92 (s, 3H).

### Step B: 4-bromo-1-methyl-1H-1,3-benzodiazole-6-carbaldehyde

To a solution of 4-bromo-1-methyl-1H-1,3-benzodiazole-6-carbonitrile (150 mg, 0.635 mmol, 1.0 eq) in Toluene (10 mL) was added dropwise DIBAL-H (1.5 N in THF) (0.63 mL, 0.953 mmol, 1.5 eq) at 0° C. under nitrogen. The reaction mixture was stirred at room temperature for 1 h, diluted with aqueous NH₄Cl solution (20 mL), and extracted with ethyl acetate (30 mL×3). The combined organic extracts were washed with brine (30 mL), dried over sodium sulfate, filtered and concentrated under reduced pressure. The residue was purified by Prep-TLC(DCM/MeOH=10/1) to give 4-bromo-1-methyl-1H-1,3-benzodiazole-6-carbaldehyde (45 mg, yield 29%) as a white solid.

LC-MS (ESI): mass calcd. for C₉H₇BrN₂O, 237.97; m/z found, 238.98 [M+H]⁺.

### Intermediate 5:3-iodo-7-(pyrrolidin-1-ylmethyl) imidazo[1,5-a]pyridine

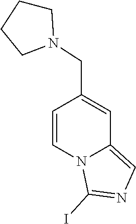 [SMILES: Ic1ncc2cc(CN3CCCC3)ccn12]

### Step A: imidazo[1,5-a]pyridin-7-yl (pyrrolidin-1-yl) methanone

To a solution of imidazo[1,5-a]pyridine-7-carboxylic acid (1 g, 6.167 mmol, 1.0 eq) and pyrrolidine (0.608 mL, 7.400 mmol, 1.2 eq) in DMF (25 mL) were added HATU (3.52 g, 9.251 mmol, 1.5 eq) and TEA (2.572 mL, 18.501 mmol, 3.0 eq). The mixture was stirred at room temperature overnight. The reaction mixture was diluted with water (30 mL) and extracted with dichloromrthane (50 mL×3). The combined organic layers were washed with brine (20 mL×3), dried over anhydrous Na₂SO₄, filtered and concentrated under reduced pressure. The residue was purified by flash column chromatography on silica gel (methanol in dichloromrthane, from 0% to 10%) to afford imidazo[1,5-a]pyridin-7-yl (pyrrolidin-1-yl) methanone (1.3 g, yield 98%) as a yellow oil.

LC-MS (ESI): mass calcd. for C₁₂H₁₃N₃O, 215.11; m/z found, 216 [M+H]⁺. ¹H NMR (400 MHZ, DMSO-d₆) δ 8.46 (s, 1H), 8.35 (d, J=7.2 Hz, 1H), 7.81 (s, 1H), 7.50 (s, 1H), 6.78 (d, J=7.2 Hz, 1H), 3.52-3.47 (m, 4H), 1.85 (s, 4H)

### Step B: 7-(pyrrolidin-1-ylmethyl) imidazo[1,5-a]pyridine

A solution of imidazo[1,5-a]pyridin-7-yl (pyrrolidin-1-yl) methanone (1 g, 4.646 mmol, 1.0 eq) and B2H₆ solution (2 N in THF) (11.614 mL, 23.228 mmol, 5.0 eq) in THF (20 mL) was stirred under N₂ at 70° C. overnight. The reaction mixture was cooled to 0° C., quenched with MeOH (20 mL) and the solution was stirred under N₂ at 70° C. for 1 hour. The reaction mixture was cooled to room temperature and concentrated, diluted with water (10 mL) and extracted with dichloromethane (50 mL×3). The combined organic layers were washed with brine (20 mL×3), dried over anhydrous Na₂SO₄, filtered and concentrated under reduced pressure. The residue was purified by flash column chromatography on silica gel (methanol in dichloromrthane, from 0% to 10%) to afford 7-(pyrrolidin-1-ylmethyl) imidazo[1,5-alpyridine (800 mg, yield 85%) as yellow oil.

LC-MS (ESI): mass calcd. for C₁₂H₁₅N₃,201.13; m/z found, 202.3 [M+H]⁺.

### Step C: 3-iodo-7-(pyrrolidin-1-ylmethyl) imidazo[1,5-a]pyridine

To a solution of 7-(pyrrolidin-1-ylmethyl) imidazo[1,5-a]pyridine (800 mg, 3.97 mmol, 1.0 eq) in THF (20 mL) was added nBuLi (2 Nin n-hexane) (2.385 mL, 5.96 mmol, 1.5 eq) at −78° C. . . . The mixture was stirred at −78° C. for 0.5 hour under N₂. Then A solution of iodine (1010 mg, 3.97 mmol, 1.0 eq) in THF (5 mL) was added dropwise to above solution. The resulting mixture was stirred at room temperature at −78° C. for 0.5 hours. The mixture was quenched with saturated aqueous NH₄Cl solution (20 mL) and extracted with ethyl acetate (40 mL×3). The combined organic layers were washed with brine (20 mL×3), dried over anhydrous Na₂SO₄, filtered and concentrated under reduced pressure. The residue was purified by Prep-TLC(DCM/MeOH=10/1) to afford 3-iodo-7-(pyrrolidin-1-ylmethyl) imidazo[1,5-alpyridine (500 mg, yield 38%) as a yellow oil.

LC-MS (ESI): mass calcd. for C₁₂H₁₄ IN3,327.02; m/z found,328 [M+H]⁺. ¹H NMR (400 MHZ, DMSO-d₆) δ 7.98 (d, J=7.2 Hz, 1H), 7.42 (d, J=5.0 Hz, 2H), 6.78 (d, J=7.2 Hz, 1H), 3.53 (s, 2H), 2.45 (s, 4H), 1.70 (s, 4H).

### Intermediate 6:3-iodo-6-(pyrrolidin-1-ylmethyl) imidazo[1,5-a]pyridine

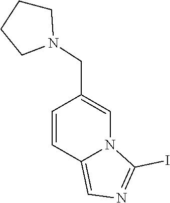 [SMILES: Ic1ncc2ccc(CN3CCCC3)cn12]

### Step A: imidazo[1,5-a]pyridin-6-yl (pyrrolidin-1-yl) methanone

To a solution of imidazo[1,5-a]pyridine-6-carboxylic acid (1 g, 6.2 mmol, 1.0 eq) and HATU (3.5 g, 9.3 mmol, 1.5 eq) in DMF (15 mL) was added TEA (2.6 mL, 18.5 mmol, 3.0 eq) and pyrrolidine (0.76 mL, 9.3 mmol, 1.5 eq) at room temperature. The reaction mixture was stirred at room temperature for 2 h. The reaction mixture was quenched with water (40 mL) and extracted with EtOAc (40 mL×3). The organic layer was washed with brine (40 mL×4), dried over anhydrous Na₂SO₄, filtered and concentrated under reduced pressure. The residue was purified by flash column chromatography on silica gel (PE/EA=1/1) to afford 1-{imidazo[1,5-a]pyridine-6-carbonyl}pyrrolidine (900 mg, yield 68%) as a yellow solid.

LC-MS (ESI): mass calcd. for C₁₂H₁₃N₃O, 215.11; m/z found, 216.12 [M+H]⁺. ¹H NMR (400 MHZ, DMSO-d₆) δ 8.67 (s, 1H), 8.42 (s, 1H), 7.57 (d, J=9.4 Hz, 1H), 7.40 (s, 1H), 6.90 (d, J=9.4 Hz, 1H), 3.53-3.47 (m, 4H), 1.88-1.83 (m, 4H).

### Step B: 6-(pyrrolidin-1-ylmethyl) imidazo[1,5-a]pyridine

To a solution of 1-{imidazo[1,5-a]pyridine-6-carbonyl}pyrrolidine (600 mg, 2.8 mmol, 1.0 eq) in THF (5 mL) was added B2H₆-Me2S complex (2 M in THF) (7 mL, 14.0 mmol, 5.0 eq) at room temperature. The reaction mixture was stirred at 70° C. for 2 h. The reaction mixture was cooled to room temperature, quenched with MeOH (5 mL), then stirred at 70° C. for 30 min. The reaction mixture was diluted with diluted aqueous HCl solution (1 N) (10 mL) and stirred for 30 min, adjusted pH to 8 with saturated NaHCO₃ solution, and extracted with EtOAc (10 mL×3). The organic layer was washed with brine (10 mL), dried over anhydrous Na₂SO₄, filtered and concentrated under reduced pressure. The residue was purified by flash column chromatography on silica gel (DCM/MeOH=5/1) to give 1-({imidazo[1,5-a]pyridin-6-yl}methyl) pyrrolidine (300 mg, yield 53%) as a yellow solid.

LC-MS (ESI): mass calcd. for C₁₂H₁₅N₃, 201.13; m/z found, 202.13 [M+H]⁺. ¹H NMR (400 MHZ, DMSO-d₆) δ 8.38 (s, 1H), 8.35 (s, 1H), 7.56 (d, J=9.4 Hz, 1H), 7.35 (s, 1H), 6.83 (d, J=9.0 Hz, 1H), 3.88-3.78 (m, 2H), 2.93-2.74 (m, 4H), 1.86-1.79 (m, 4H).

### Step C: 3-iodo-6-(pyrrolidin-1-ylmethyl) imidazo[1,5-a]pyridine

To a solution of 1-({imidazo[1,5-a]pyridin-6-yl}methyl) pyrrolidine (300 mg, 1.5 mmol, 1.0 eq) in anhydrous THF (5 mL) was added dropwise n-BuLi (1.6 M in hexane) (0.894 mL, 2.3 mmol, 1.5 eq) under N₂ at −78° C. The reaction mixture was stirred at −78° C. for 1 h. A solutinas stirre of I₂ (661.2 mg, 1.5 mmol, 1.0 eq) in THF (5 mL) was added dropwise to above mixture and The resulting reaction mixture wd at −78° C. for 0.5 h. The reaction mixture was quenched with water (40 mL) and extracted with EtOAc (40 mL×3). The organic layer was washed with brine (40 mL×4), dried over anhydrous Na₂SO₄, filtered and concentrated under reduced pressure. The residue was purified by flash column chromatography on silica gel (DCM/MeOH=5/1) to afford 1-({3-iodoimidazo[1,5-a]pyridin-6-yl}methyl) pyrrolidine (150 mg, yield 31%) as a yellow solid.

LC-MS (ESI): mass calcd. for C₁₂H₁₄ IN3, 327.02; m/z found, 328.03 [M+H]⁺. ¹HNMR (400 MHZ, DMSO-d₆) δ 7.90 (s, 1H), 7.53 (d, J=9.4 Hz, 1H), 7.48 (s, 1H), 6.85 (d, J=9.0 Hz, 1H), 3.61 (s, 2H), 3.42 (s, 2H), 3.04-2.89 (m, 2H), 1.72 (s, 4H).

### Intermediate 7:5-chloroimidazo[1,5-a]pyridine-8-carbaldehyde

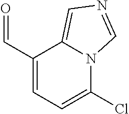 [SMILES: O=Cc1ccc(Cl)n2cncc12]

### Step A: 3-bromo-2-(bromomethyl)-6-chloropyridine

To a solution of 3-bromo-6-chloro-2-methylpyridine (8 g, 38.747 mmol, 1.0 eq) in Carbon tetrachloride (130 mL) were added BPO (0.94 g, 3.875 mmol, 0.1 eq) and NBS (8.97 g, 50.371 mmol, 1.3 eq) at room temperature. The reaction mixture was heated to 95° C. overnight. After cooled to room temperature, the reaction mixture was quenched with water (200 mL) and extracted with ethyl acetate (200 mL×3). The combined organic extracts were washed with water (200 mL×3), dried over sodium sulfate, filtered and concentrated under reduced pressure. The residue was purified by flash column chroatography on silica gel (PE/EA=80/1) to provide 3-bromo-2-(bromomethyl)-6-chloropyridine (7.1 g, yield 55%) as a yellow oil.

LC-MS (ESI): mass calcd. for C₆H₄Br2ClN, 282.84; m/z found, 283.85 [M+H]⁺.

### Step B: 2-(azidomethyl)-3-bromo-6-chloropyridine

To a solution of 3-bromo-2-(bromomethyl)-6-chloropyridine (7.1 g, 24.880 mmol, 1.0 eq) in DMF (100 mL) was added sodium azide (3.23 g, 49.760 mmol, 2.0 eq) at room temperature. The reaction mixture was stirred at 30° C. for 1 h. The reaction was quenched with water (200 mL) and extracted with ethyl acetate (150 mL×3). The combined organic extracts were washed with brine (100 mL×4), dried over anhydrous sodium sulfate, filtered and concentrated under reduced pressure to give 2-(azidomethyl)-3-bromo-6-chloropyridine (6.0 g, yield 83%) as a yellow oil. The oil was directly used in the next step without further purification.

LC-MS (ESI): mass calcd. for C₆H₄BrClN₄, 245.93; m/z found, 246.94 [M+H]⁺.

### Step C: (3-bromo-6-chloropyridin-2-yl) methanamine

To a solution of 2-(azidomethyl)-3-bromo-6-chloropyridine (6.0 g, 24.244 mmol, 1.0 eq) in THF (70 mL) and H₂O (8 mL) was added PPh3 (9.54 g, 36.366 mmol, 1.5 eq) at room temperature. The reaction mixture was stirred at 50° C. for 2 h. After evaporation, the residue was diluted with water (30 mL), acidified to pH 2-3 with aqueous HCl solution (2 N), and extracted with DCM (50 mL×2). After discarded off organic layer, the aqueous phase was concentrated under reduced pressure to give (3-bromo-6-chloropyridin-2-yl) methanamine hydrochloride (3.35 g, yield 56%) as a red oil.

LC-MS (ESI): mass calcd. for C₆H₆BrClN₂, 219.94; m/z found, 220.95 [M+H]⁺. ¹H NMR (400 MHZ, DMSO-d₆) δ 8.61 (s, 3H), 8.24 (d, J=8.4 Hz, 1H), 7.56 (d, J=8.4 Hz, 1H), 4.24 (s, 2H).

### Step D: N-[(3-bromo-6-chloropyridin-2-yl)methyl] formamide

To a solution of (3-bromo-6-chloropyridin-2-yl) methanamine (3.35 g, 15.125 mmol, 1.0 eq) in ethyl formate (80 mL) were added NaHCO₃ (2.54 g, 30.250 mmol, 2.0 eq) and Triethylamine (10.5 mL, 75.624 mmol, 5.0 eq) at room temperature. The reaction mixture was stirring at 70° C. overnight. After cooled to room temperature, the mixture was filtered and the filtrate was concentrated under reduced pressure to give N-[(3-bromo-6-chloropyridin-2-yl)methyl]formamide (3.0 g, yield 64%) as a pink solid, which was directly used in next step.

LC-MS (ESI): mass calcd. for C₇H₆BrClN₂O, 247.94; m/z found, 248.94 [M+H]⁺.

### Step E: 8-bromo-5-chloroimidazo[1,5-a]pyridine

To a solution of N-[(3-bromo-6-chloropyridin-2-yl)methyl]formamide (3.0 g, 12.024 mmol, 1.0 eq) in dioxane (60 mL) was added POCl₃ (2.2 mL, 24.048 mmol, 2.0 eq) at room temperature. The reaction mixture was stirred at 115° C. for 3 h. After cooled to room temperature, the mixture was slowly quenched with aqueous NaHCO₃ solution and extracted with EtOAc (50 mL×2). The organic layers were washed with brine (50 mL), dried over anhydrous Na₂SO₄, filtered and concentrated under reduced pressure. The crude product was purified by flash column chroatography on silica gel (PE/EA=10/1) to provide 8-bromo-5-chloroimidazo[1,5-a]pyridine (1.57 g, yield 56%) as a yellow solid.

LC-MS (ESI): mass calcd. for C₇H₄BrClN₂, 229.92; m/z found, 230.93 [M+H]⁺.

### Step F: methyl 5-chloroimidazo[1,5-a]pyridine-8-carboxylate

To a solution of 8-bromo-5-chloroimidazo[1,5-a]pyridine (0.5 g, 2.160 mmol, 1.0 eq) in DMF (20 mL) and MeOH (20 mL) were added Triethylamine (1.5 mL, 10.800 mmol, 5 eq) and Pd(dppf)Cl₂ (0.16 g, 0.216 mmol, 0.1 eq) at room temperature. The reaction mixture was stirred under CO (1 atm) at 80° C. for 6 hr. After cooled to room temperature, the reaction mixture was diluted with water (50 mL) and extracted with ethyl acetate (50 mL×3). The combined organic extracts were washed with water (30 mL×2) and brine (30 mL×2), dried over anhydrous sodium sulfate, filtered and concentrated under reduced pressure. The residue was purified by flash column chroatography on silica gel (PE/EA=6/1) to provide methyl 5-chloroimidazo[1,5-a]pyridine-8-carboxylate (0.21 g, yield 46%) as a yellow solid.

LC-MS (ESI): mass calcd. for C₉H₇ClN₂O₂, 210.02; m/z found, 211.03 [M+H]⁺.

### Step G: {5-chloroimidazo[1,5-a]pyridin-8-yl}methanol

To a solution of methyl 5-chloroimidazo[1,5-a]pyridine-8-carboxylate (210 mg, 0.997 mmol, 1.0 eq) in THF (8 mL) was added dropwise DIBAL-H (1 M) (3 mL, 2.991 mmol, 3 eq) at −70° C. under nitrogen. Then the reaction mixture was stirred at 0° C. for 1 h, slowly diluted with saturated aqueous NH₄Cl solution (10 mL), and extracted with ethyl acetate (20 mL×3). The combined organic extracts were washed with water (10 mL) and brine (10 mL), dried over anhydrous sodium sulfate, filtered, and concentrated under reduced pressure. The residue was purified by flash column chroatography on silica gel (100% EA) to provide {5-chloroimidazo[1,5-a]pyridin-8-ylmethanol (100 mg, yield 55%) as a yellow solid.

LC-MS (ESI): mass calcd. for C₈H₇ClN₂O, 182.02; m/z found, 183.03 [M+H]⁺.

### Step H: 5-chloroimidazo[1,5-a]pyridine-8-carbaldehyde

To a solutio of {5-chloroimidazo[1,5-a]pyridin-8-yl}methanol (100 mg, 0.548 mmol, 1.0 eq) in DCM (8 mL) was added Dess-Martin periodinane (302.16 mg, 0.712 mmol, 1.5 eq) at 0° C. and the mixture was stirred at room temperature for 2 h. The mixture was diluted with DCM (30 mL), washed with brine (10 mL), dried over anhydrous Na₂SO₄, filtered and concentrated under reduced pressure. The crude was purified by Prep-TLC(100% EA) to give 5-chloroimidazo[1,5-a]pyridine-8-carbaldehyde (90 mg, yield 87%) as a yellow solid. LC-MS (ESI): mass calcd. for C₈H₅ClN₂O, 180.01; m/z found, 181.02 [M+H]⁺.

### Intermediate 8:6-chloro-1-methyl-1H-pyrrolo[2,3-b]pyridine-4-carbaldehyde

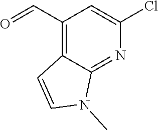

### Step A: methyl 6-chloro-1-methyl-1H-pyrrolo[2,3-b]pyridine-4-carboxylate

To a solution of methyl 6-chloro-1H-pyrrolo[2,3-b]pyridine-4-carboxylate (800 mg, 3.8 mmol, 1.0 eq) in DMF (10 mL) were added NaH (60% suspend in oil) (341.8 mg, 5.7 mmol, 1.5 eq) and CH₃I (0.36 mL, 5.7 mmol, 1.5 eq) at 0° C. The reaction mixture was stirred at room temperature for 2 h. The reaction mixture was quenched with water (40 mL) and extracted with EtOAc (40 mL×3). The organic layer was washed with brine (40 mL×3), dried over anhydrous Na₂SO₄, filtered and concentrated under reduced pressure. The residue was purified by flash column chromatography on silica gel (PE/EA=10/1) to afford methyl 6-chloro-1-methyl-1H-pyrrolo[2,3-b]pyridine-4-carboxylate (700 mg, yield 82%) as a yellow solid.

LC-MS (ESI, m/z): mass calcd. for C₁₀H₉ClN₂O₂, 224.04; found, 224.9 [M+H]⁺.

### Step B: (6-chloro-1-methyl-1H-pyrrolo[2,3-b]pyridin-4-yl) methanol

To a solution of methyl 6-chloro-1-methyl-1H-pyrrolo[2,3-b]pyridine-4-carboxylate (700 mg, 3.1 mmol, 1.0 eq) in anhydrous THF (10 mL) was added LiAlH4 (118.3 mg, 3.1 mmol, 1.0 eq) in portions at 0° C. The reaction mixture was stirred at 0° C. for 20 min. The reaction mixture was slowly quenched with aqueous NaOH solution (1 N) (40 mL) and extracted with EtOAc (40 mL×3). The organic layer was washed with brine (40 mL×4), dried over anhydrous Na₂SO₄, filtered and concentrated under reduced pressure. The residue was purified by flash column chromatography on silica gel (PE/EA=1/1) to afford {6-chloro-1-methyl-1H-pyrrolo[2,3-b]pyridin-4-yl}methanol (500 mg, yield 82%) as a yellow solid.

LC-MS (ESI, m/z): mass calcd. for C₉H₉ClN₂O, 196.04; found, 196.9 [M+H]⁺.

### Step C: 6-chloro-1-methyl-1H-pyrrolo[2,3-b]pyridine-4-carbaldehyde

To a solution of {6-chloro-1-methyl-1H-pyrrolo[2,3-b]pyridin-4-yl}methanol (500 mg, 2.5 mmol, 1.0 eq) in DMSO (15 mL) was added IBX (2.1 g, 7.5 mmol, 3.0 eq) in portions at room temperature. The reaction mixture was stirred at room temperature for 1 h. The reaction mixture was quenched with water (40 mL) and extracted with EtOAc (40 mL×3). The organic layer was washed with brine (40 mL×4), dried over anhydrous Na₂SO₄, filtered and concentrated under reduced pressure. The residue was purified by flash column chromatography on silica gel (PE/EA=2/1) to afford 6-chloro-1-methyl-1H-pyrrolo[2,3-b]pyridine-4-carbaldehyde (350 mg, yield 71%) as a yellow solid.

LC-MS (ESI, m/z): mass calcd. for C₉H₇ClN₂O, 194.02; found, 195.2 [M+H]⁺.

### Intermediate 9:6-chloro-1H-pyrrolo[2,3-b]pyridine-4-carbaldehyde

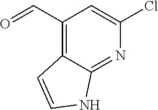 [SMILES: O=Cc1cc(Cl)nc2[nH]ccc12]

### Step A: (6-chloro-1H-pyrrolo[2,3-b]pyridin-4-yl) methanol

To a solution of methyl 6-chloro-1H-pyrrolo[2,3-b]pyridine-4-carboxylate (120 mg, 570 μmol, 1.0 eq) in THF (5.0 mL) was added LiAlH4 (21.6 mg, 570 μmol, 1.0 eq) in portions at 0° C. The reaction mixture was stirred at 0° C. for 20 min. The reaction mixture was slowly quenched with aqueous NaOH solution (1 N) (5 mL) and extracted with EtOAc (5 mL×3). The organic layer was washed with brine (5 mL×2), dried over anhydrous Na₂SO₄, filtered and concentrated under reduced pressure. The residue was purified by flash column chromatography on silica gel (PE/EA=1/1) to afford (6-chloro-1H-pyrrolo[2,3-b]pyridin-4-yl) methanol (100 mg, yield 96%) as a yellow solid.

LC-MS (ESI): mass calcd. for C₈H₇ClN₂O, 182.02; m/z found, 183.02 [M+H]⁺.

### Step B: 6-chloro-1H-pyrrolo[2,3-b]pyridine-4-carbaldehyde

To a solution of (6-chloro-1H-pyrrolo[2,3-b]pyridin-4-yl) methanol (100 mg, 548 μmol, 1.0 eq) in DMSO (5.0 mL) were added IBX (383 mg, 1.4 mmol, 2.5 eq) in portions at room temperature. The reaction mixture was stirred at room temperature for 1 h. The reaction mixture was quenched with water (10 mL) and extracted with EtOAc (10 mL×3). The organic layer was washed with brine (10 mL×4), dried over anhydrous Na₂SO₄, filtered and concentrated under reduced pressure. The residue was purified by flash column chromatography on silica gel (PE/EA=2/1) to afford 6-chloro-1H-pyrrolo[2,3-b]pyridine-4-carbaldehyde (80 mg, yield 81%) as a yellow solid.

LC-MS (ESI): mass calcd. for C₈H₅ClN₂O, 180.01; m/z found, 181.01 [M+H]⁺.

### Intermediate 10:6-chloro-1-ethyl-1H-pyrrolo[2,3-b]pyridine-4-carbaldehyde

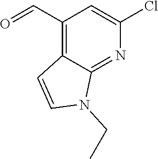

### Step A: methyl 6-chloro-1-ethyl-1H-pyrrolo[2,3-b]pyridine-4-carboxylate

To a stirred solution of methyl 6-chloro-1H-pyrrolo[2,3-b]pyridine-4-carboxylate (500 mg, 2.37 mmol, 1.0 eq) in DMF (6.00 mL) at 0° C. was added sodium hydride (60% suspend in oil) (0.19 g, 4.75 mmol, 2.0 eq) and the reaction mixture was stirred at 0° C. for 1 hour. Then iodoethane (444 mg, 2.85 mmol, 1.2 eq) was added to above mixture and the reaction mixture was stirred at 25° C. for 30 min under nitrogen atmosphere. The reaction mixture was quenched with saturated aqueous NH₄Cl solution (20 mL) and extracted with EtOAc (30 mL×3). The combined organic phases were washed with brine (30 mL×4), dried over anhydrous sodium sulfate, filtered and concentrated under reduced pressure. The residue was purified by flash column chromatography on silica gel (PE/EtOAc=10/1) to obtain methyl 6-chloro-1-ethyl-1H-pyrrolo[2,3-b]pyridine-4-carboxylate (215 mg, yield 38%) as a white solid.

LC-MS (ESI): mass calcd. for C₁₁H₁₁ClN₂O₂, 238.05; m/z found, 239.05 [M+H]⁺.

### Step B: (6-chloro-1-ethyl-1H-pyrrolo[2,3-b]pyridin-4-yl) methanol

To a solution of methyl 6-chloro-1-ethyl-1H-pyrrolo[2,3-b]pyridine-4-carboxylate (215 mg, 901 μmol, 1.0 eq) in THF (4.00 mL) at 0° C. was added Lithium Aluminum Hydride (34.2 mg, 901 μmol, 1.0 eq) and the reaction mixture was stirred at 0° C. for 5 min under nitrogen atmosphere. The reaction mixture was quenched with saturated NaOH solution (5 mL) and extracted with EtOAc (20 mL×3). The combined organic phases were concentrated under reduced pressure to obtain (6-chloro-1-ethyl-1H-pyrrolo[2,3-b]pyridin-4-yl) methanol (170 mg, yield 89.6%) as a red oil. LC-MS (ESI): mass calcd. for C₁₀H₁₁ClN₂O, 210.06; m/z found, 211.06 [M+H]⁺.

### Step C: 6-chloro-1-ethyl-1H-pyrrolo[2,3-b]pyridine-4-carbaldehyde

To a stirred solution of (6-chloro-1-ethyl-1H-pyrrolo[2,3-b]pyridin-4-yl) methanol (170 mg, 807 μmol, 1.0 eq) in DMSO (4.00 mL) was added IBX (678 mg, 2.42 mmol, 3.0 eq) and the reaction mixture was stirred at 30° C. for 20 min under nitrogen atmosphere. The reaction mixture was quenched with water (15 mL) and extracted with EtOAc (20 mL×3). The combined organic phases were washed with brine (20 mL×4), dried over anhydrous sodium sulfate, filtered and concentrated under reduced pressure. The residue was purified by flash column chromatography on silica gel (PE/EtOAc=10/1) to obtain 6-chloro-1-ethyl-1H-pyrrolo[2,3-b]pyridine-4-carbaldehyde (110 mg, yield 65%) as a yellow solid.

LC-MS (ESI): mass calcd. for C₁₀H₉ClN₂O, 208.04; m/z found, 209.04 [M+H]⁺. ¹H NMR (400 MHZ, DMSO-d₆) δ 10.28 (s, 1H), 7.90 (d, J=3.4 Hz, 1H), 7.70 (s, 1H), 7.00 (d, J=3.4 Hz, 1H), 4.31 (q, J=7.2 Hz, 2H), 1.39 (t, J=7.2 Hz, 3H).

### Intermediate 11:5-chloro-1H-pyrrolo[3,2-b]pyridine-7-carbaldehyde

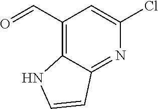 [SMILES: O=Cc1cc(Cl)nc2cc[nH]c12]

### Step A: (5-chloro-1H-pyrrolo[3,2-b]pyridin-7-yl) methanol

To a solution of methyl 5-chloro-1H-pyrrolo[3,2-b]pyridine-7-carboxylate (350 mg, 1.66 mmol, 1.0 eq) in THF (10.0 mL) was added LiAlH4 (94.6 mg, 2.49 mmol, 1.5 eq) at 0° C. and the mixture was stirred at 0° C. for 30 min. The mixture was quenched with Na₂SO₄·10H₂O and filtered. The filtrate was concentrated under reduced pressure to afford (5-chloro-1H-pyrrolo[3,2-b]pyridin-7-yl) methanol (250 mg, yield 82%) as a light yellow solid.

LC-MS (ESI): mass calced for C₈H₇ClN₂O 182.6; m/z found, 183.2 [M+H]⁺

### Step B: 5-chloro-1H-pyrrolo[3,2-b]pyridine-7-carbaldehyde

To a solution of (5-chloro-1H-pyrrolo[3,2-b]pyridin-7-yl) methanol (250 mg, 1.37 mmol, 1.0 eq) in DMSO (5.00 mL) was added IBX (575 mg, 2.05 mmol, 1.5 eq). The mixture was stirred at 30° C. for 3 h. The reaction mixture was quenched with ice water (20 mL) and exacted with EA (15 mL×3). The organic layer was washed with brine (10 mL×4), dried over anhydrous Na₂SO₄, filtered and concentrated under reduced pressure. The residue was purified by flash column chromatography on silica gel (ethyl acetate in petroleum ether, from 0 to 30%) to afford 5-chloro-1H-pyrrolo[3,2-b]pyridine-7-carbaldehyde (180 mg, yield 73%) as a light yellow solid.

LC-MS (ESI): mass calced for C₈H₅ClN₂O 180.01; m/z found, 181.1 [M+H]⁺

### Intermediate 12:5-chloro-1-methyl-1H-pyrrolo[3,2-b]pyridine-7-carbaldehyde

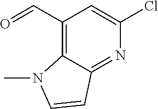

### Step A: methyl 5-chloro-1-methyl-1H-pyrrolo[3,2-b]pyridine-7-carboxylate

To a mixture of methyl 5-chloro-1H-pyrrolo[3,2-b]pyridine-7-carboxylate (150 mg, 0.71 mmol, 1.0 eq) in DMF (3.00 mL) was added NaH (60% suspend in oil) (51 mg, 2.14 mmol, 3.0 eq) at 0° C. and the mixture was stirred for 1 hours under N₂ atmosphere. Then MeI (355 mg, 2.14 mmol, 3.0 eq) was added to above mixture and the resulting mixture was stirred at 0° C. for 2 hours. The reaction mixture was quenched with saturated aqueous NH₄Cl solution (20 mL) and extracted with EtOAc (15 mL×3). The organic layer was washed with brine (20 mL×4), dried over anhydrous Na₂SO₄, filtered and concentrated under reduced pressure. The residue was purified by flash column chromatography on silica gel (EtOAc in PE, from 0 to 50%) to afford methyl 5-chloro-1-methyl-1H-pyrrolo[3,2-b]pyridine-7-carboxylate (50.0 mg, yield 30%) as a white solid.

LC-MS (ESI): mass calced for C₁₀H₉ClN₂O₂ 223.2; m/z found, 225.2 [M+H]⁺

### Step B: (5-chloro-1-methyl-1H-pyrrolo[3,2-b]pyridin-7-yl) methanol

To a solution of methyl 5-chloro-1-methyl-1H-pyrrolo[3,2-b]pyridine-7-carboxylate (370 mg, 1.65 mmol, 1.0 eq) in THF (10.0 mL) was added LiAlH4 (93.8 mg, 2.47 mmol, 1.5 eq) at 0° C. The mixture was stirred at 0° C. for 30 min, then quenched with Na₂SO₄·10H₂O, stirred for 10 min at room temperature. After filtration, the filtare was concentrated under reduced pressure to afford (5-chloro-1-methyl-1H-pyrrolo[3,2-b]pyridin-7-yl) methanol (280 mg, 1.42 mmol, 86.5%) as a light yellow solid.

LC-MS (ESI): mass calced for C₉H₉ClN₂O 196.6; m/z found, 197 [M+H]⁺

### Step C: 5-chloro-1-methyl-1H-pyrrolo[3,2-b]pyridine-7-carbaldehyde

To a solution of (5-chloro-1-methyl-1H-pyrrolo[3,2-b]pyridin-7-yl) methanol (280 mg, 1.42 mmol, 1.0 eq) in DMSO (8.00 mL) was added IBX (598 mg, 2.14 mmol, 1.5 eq) at 0° C. The mixture was stirred at room temperature for 30 min. The reaction mixture was quenched with ice water (10 mL) and exacted with EA (15 mL×3). The organic layer was washed with brine (10 mL×4), dried over anhydrous Na₂SO₄, filtered and concentrated under reduced pressure. The residue was purified by flash column chromatography on silica gel (EA in PE, from 0 to 30%) to afford 5-chloro-1-methyl-1H-pyrrolo[3,2-b]pyridine-7-carbaldehyde (160 mg, yield 58%) as a light yellow solid.

LC-MS (ESI): mass calced for C₉H₇ClN₂O 194.02; m/z found, 195.2 [M+H]⁺. ¹H NMR (400 MHZ, DMSO-d₆) δ 10.50 (s, 1H), 7.86 (d, J=3.4 Hz, 1H), 7.60 (s, 1H), 6.70 (d, J=3.4 Hz, 1H), 4.11 (s, 3H)

### Intermediate 13:3-(1-oxo-5-(4,4,5,5-tetramethyl-1,3,2-dioxaborolan-2-yl) isoindolin-2-yl) piperidine-2,6-dione

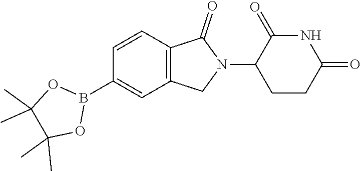 [SMILES: CC1(C)OB(c2ccc3c(c2)CN(C2CCC(=O)NC2=O)C3=O)OC1(C)C]

### Step A: 3-(5-bromo-1-oxoisoindolin-2-yl) piperidine-2,6-dione

To a solution of methyl 4-bromo-2-(bromomethyl)benzoate (20 g, 65.1 mmol, 1.0 eq) in ACN (500 mL) were added 3-aminopiperidine-2,6-dione hydrochloride (10.7 g, 65.1 mmol, 1.0 eq) and DIPEA (25.2 g, 195.3 mmol, 3.0 eq) at room temperature. The reaction mixture was stirred at 85° C. for 12 h. After evaporation, the residue was diluted with a mixture ACN and H₂O (160 mL, 3/1 v/v) and filtrated to give a bule solid. The solid was dried to give 3-(5-bromo-1-oxoisoindolin-2-yl) piperidine-2,6-dione (10.5 g, yield 50%).

LC-MS (ESI): mass calcd. for C₁₃H₁₁BrN₂O₃, 322.00; m/z found, 323.00 [M+H]⁺. ¹H NMR (400 MHZ, DMSO-d₆) δ 11.00 (s, 1H), 7.89 (s, 1H), 7.73-7.66 (m, 2H), 5.14-5.09 (m, 1H), 4.47 (d, J=16.8 Hz, 1H), 4.34 (d, J=16.8 Hz, 1H), 3.02-2.77 (m, 1H), 2.62-2.58 (m, 1H), 2.45-2.36 (m, 1H), 2.13-1.92 (m, 1H).

### Step B: 3-(1-oxo-5-(4,4,5,5-tetramethyl-1,3,2-dioxaborolan-2-yl) isoindolin-2-yl) piperidine-2,6-dione

To a solution of methyl 3-(5-bromo-1-oxoisoindolin-2-yl) piperidine-2,6-dione (10.5 g, 32.5 mmol, 1.0 eq) in anhydrous dioxane (150 mL) were added 4,4,4′,4′,5,5,5′,5′-octamethyl-2,2′-bi (1,3,2-dioxaborolane) (9.9 g, 39.0 mmol, 1.2 eq), Pd(dppf)Cl₂ (1.2 g, 1.62 mmol, 0.05 eq), and KOAc (9.5 g, 97.5 mmol, 3.0 eq) at room temperature. The reaction mixture was stirred at 100° C. under N₂ for 12 h. After evaporation, the residue was diluted with H₂O (150 mL) and filtrated to give a bule solid. The blue solid was washed with EA (50 mL×3) and dried to give 3-(1-oxo-5-(4,4,5,5-tetramethyl-1,3,2-dioxaborolan-2-yl) isoindolin-2-yl) piperidine-2,6-dione (9.2 g, yield 71%) as a blue solid.

LC-MS (ESI): mass calcd. for C₁₉H₂₃BN₂O₅, 370.17.; m/z found, 371.21 [M+H]⁺. ¹H NMR (400 MHZ, DMSO-d₆) δ 10.99 (s, 1H), 7.90 (s, 1H), 7.80 (d, J=7.6 Hz, 1H), 7.73 (d, J=7.6 Hz, 1H), 5.15-5.10 (m, 1H), 4.47 (d, J=16.8 Hz, 1H), 4.35 (d, J=16.8 Hz, 1H), 3.08-2.80 (m, 1H), 2.62-2.58 (m, 1H), 2.45-2.33 (m, 1H), 2.18-1.94 (m, 1H), 1.24 (s, 12H).

### Intermediate 14:3-(4-fluoro-1-oxo-5-(4,4,5,5-tetramethyl-1,3,2-dioxaborolan-2-yl) isoindolin-2-yl) piperidine-2,6-dione

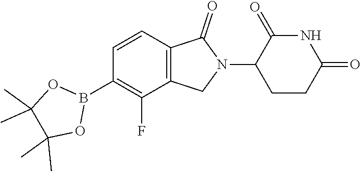 [SMILES: CC1(C)OB(c2ccc3c(c2F)CN(C2CCC(=O)NC2=O)C3=O)OC1(C)C]

The title compound was prepared in a manner analogous to Intermediate 13 by brominating of methyl 4-bromo-3-fluoro-2-methylbenzoate, coupling with 3-aminopiperidine-2,6-dione, cylcization and then boronation with 4,4,4′,4′,5,5,5′,5′-octamethyl-2,2′-bi (1,3,2-dioxaborolane).

LC-MS (ESI): mass calcd. for C₁₉H₂₂BFN₂O₅, 388.16; m/z found, 389.3 [M+H]⁺.

### Intermediate 15:3-(6-fluoro-1-oxo-5-(4,4,5,5-tetramethyl-1,3,2-dioxaborolan-2-yl) isoindolin-2-yl) piperidine-2,6-dione

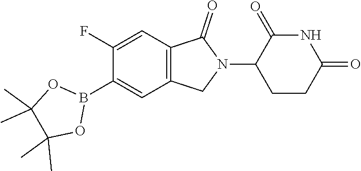 [SMILES: CC1(C)OB(c2cc3c(cc2F)C(=O)N(C2CCC(=O)NC2=O)C3)OC1(C)C]

The title compound was prepared in a manner analogous to Intermediate 13 by brominating of methyl 4-bromo-5-fluoro-2-methylbenzoate, coupling with 3-aminopiperidine-2,6-dione, cylcization and then boronation with 4,4,4′,4′,5,5,5′,5′-octamethyl-2,2′-bi (1,3,2-dioxaborolane).

LC-MS (ESI): mass calcd. for C₁₉H₂₂BFN₂O₅, 388.20; m/z found, 389.1 [M+H]⁺. ¹HNMR (400 MHz, DMSO-d₆) δ 11.02 (s, 1H), 7.89 (d, J=4.0 Hz, 1H), 7.45 (d, J=8.0 Hz, 1H), 5.15-5.11 (m, 1H), 4.45 (d, J=17.6 Hz, 1H), 4.33 (d, J=17.6 Hz, 1H), 2.93-2.91 (m, 1H), 2.62-2.58 (m, 1H), 2.41-2.36 (m, 1H), 2.03-1.99 (m, 1H), 1.32 (s, 12H).

### Intermediate 16:3-(7-fluoro-1-oxo-5-(4,4,5,5-tetramethyl-1,3,2-dioxaborolan-2-yl) isoindolin-2-yl) piperidine-2,6-dione

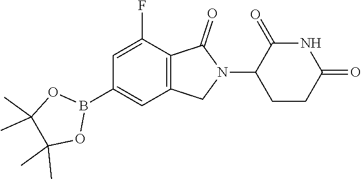 [SMILES: CC1(C)OB(c2cc(F)c3c(c2)CN(C2CCC(=O)NC2=O)C3=O)OC1(C)C]

The title compound was prepared in a manner analogous to Intermediate 13 by brominating of methyl 4-bromo-6-fluoro-2-methylbenzoate, coupling with 3-aminopiperidine-2,6-dione, cylcization and then boronation with 4,4,4′,4′,5,5,5′,5′-octamethyl-2,2′-bi (1,3,2-dioxaborolane).

LC-MS (ESI): mass calcd. for C₁₉H₂₂BFN₂O₅, 388.20; m/z found, 389.1 [M+H]⁺. ¹H NMR (400 MHZ, DMSO-d₆) δ 11.01 (s, 1H), 7.71 (s, 1H), 7.39 (d, J=8.0 Hz, 1H), 5.12-5.07 (m, 1H), 4.49 (d, J=17.6 Hz, 1H), 4.37 (d, J=17.6 Hz, 1H), 2.94-2.91 (m, 1H), 2.62-2.59 (m, 1H), 2.50-2.48 (m, 1H), 2.01-1.99 (m, 1H), 1.32 (s, 12H).

### Intermediate 17:3-(4-methoxy-1-oxo-5-(4,4,5,5-tetramethyl-1,3,2-dioxaborolan-2-yl) isoindolin-2-yl) piperidine-2,6-dione

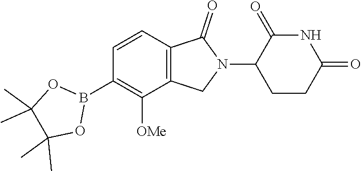 [SMILES: COc1c(B2OC(C)(C)C(C)(C)O2)ccc2c1CN(C1CCC(=O)NC1=O)C2=O]

The title compound was prepared in a manner analogous to Intermediate 13 by brominating of methyl 4-bromo-3-methoxy-2-methylbenzoate, coupling with 3-aminopiperidine-2,6-dione, cylcization and then boronation with 4,4,4′,4′,5,5,5′,5′-octamethyl-2,2′-bi (1,3,2-dioxaborolane).

### Intermediate 18:3-(6-methoxy-1-oxo-5-(4,4,5,5-tetramethyl-1,3,2-dioxaborolan-2-yl) isoindolin-2-yl) piperidine-2,6-dione

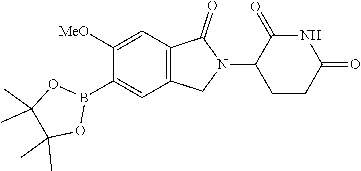 [SMILES: COc1cc2c(cc1B1OC(C)(C)C(C)(C)O1)CN(C1CCC(=O)NC1=O)C2=O]

The title compound was prepared in a manner analogous to Intermediate 13 by brominating of methyl 4-bromo-5-methoxy-2-methylbenzoate, coupling with 3-aminopiperidine-2,6-dione, cylcization and then boronation with 4,4,4′,4′,5,5,5′,5′-octamethyl-2,2′-bi (1,3,2-dioxaborolane).

### Intermediate 19:3-(7-methoxy-1-oxo-5-(4,4,5,5-tetramethyl-1,3,2-dioxaborolan-2-yl) isoindolin-2-yl) piperidine-2,6-dione

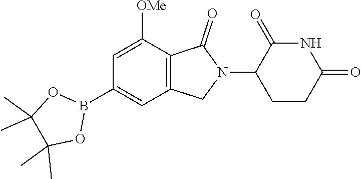 [SMILES: COc1cc(B2OC(C)(C)C(C)(C)O2)cc2c1C(=O)N(C1CCC(=O)NC1=O)C2]

The title compound was prepared in a manner analogous to Intermediate 13 by brominating of methyl 4-bromo-6-methoxy-2-methylbenzoate, coupling with 3-aminopiperidine-2,6-dione, cylcization and then boronation with 4,4,4′,4′,5,5,5′,5′-octamethyl-2,2′-bi (1,3,2-dioxaborolane).

### Intermediate 20:2-(3-chloro-4-methylphenyl) ethan-1-amine

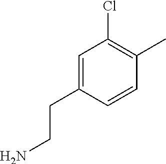

### Step A: (3-chloro-4-methylphenyl)methyl methanesulfonate

To a solution of (3-chloro-4-methylphenyl) methanol (1 g, 6.385 mmol, 1.0 eq) and TEA (1.8 mL, 12.77 mmol, 2.0 eq) in DCM (15 mL) was added dropwise MsCl (0.741 mL, 9.578 mmol, 1.5 eq) at 0° C. the mixture was stirred at room temperature for 2 h. The residue was poured into water (30 mL) and extracted with DCM (30 mL×3). The organic layer was washed with brine (30 mL), dried over MgSO₄, filtered and concentrated under reduced pressure. The residue was purified by flash column chromatography on silica gel (ethyl acetate in petroleum ether, 10%) to give (3-chloro-4-methylphenyl)methyl methanesulfonate (0.6 g, yield 36%) as a yellow oil.

### Step B: 2-(3-chloro-4-methylphenyl) acetonitrile

To a solution of (3-chloro-4-methylphenyl)methyl methanesulfonate (500 mg, 2.130 mmol, 1.0 eq) in DMF (1 mL) was added NaCN (0.131 mL, 4.261 mmol, 2.0 eq) and the mixture was stirring at 50° C. overnight. The mixture was poured into water (6 mL) and extracted with DCM (15 mL×3). The organiclayer was washed with brine (15 mL×4), dried over MgSO₄, filtered and concentrated under reduced pressure. The residue was purified by flash column chromatography on silica gel (ethyl acetate in petroleum ether, 10%) to give 2-(3-chloro-4-methylphenyl) acetonitrile (300 mg, yield 76%) as a yellow oil.

### Step C: 2-(3-chloro-4-methylphenyl) ethan-1-amine

To a solution of 2-(3-chloro-4-methylphenyl) acetonitrile (300 mg, 1.811 mmol, 1.0 eq) in THF (5 mL) was added BH3.THF (1 M in THF) (5.4 mL, 5.433 mmol, 3.0 eq) and the mixture was stirring at 40° C. overnight. The residue was quenched with MeOH (6 mL) and stirred for 30 min. Then con. HCl (3 mL) was added to above mixture and stirred for 30 min. After evaporation, the residue was diluted with H₂O (10 mL), adjusted to pH 9-10, and extracted with DCM (15 mL×3). The organic layer was dried over MgSO₄, filtered and concentrated under reduced pressure. The crude product was purified by flash column chromatography on silica gel (ethyl acetate in petroleum ether, 40%) to give 2-(3-chloro-4-methylphenyl) ethan-1-amine (100 mg, yield 29.3%) as a yellow oil.

LC-MS (ESI): mass calcd. for C₉H₁₂ClN, 169.07; m/z found, 170.3 [M+H]⁺.

### Intermediate 21: (1-methyl-1H-indol-7-yl) methanamine

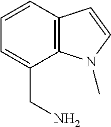

### Step A: 1-methyl-1H-indole-7-carbaldehyde

To a solution of 1H-indole-7-carbaldehyde (1.4 g, 9.645 mmol, 1.0 eq) in DMF (10 mL) was added NaH (60% suspend in oil) (0.46 g, 11.574 mmol, 1.2 eq) and the mixture was stirred for 20 min. Then iodomethane (1.64 g, 11.574 mmol, 1.2 eq) was added dropwise to above mixture and the mixture is stirred for 12 h at room temperature. The reaction mixture was quenched with saturated aqueous NH₄Cl solution (30 mL) and extracted with EA (40 mL×3). The combined organic phases were washed with brine (30 mL×4), dried over anhydrous Na₂SO₄, filtered and concentrated under reduced pressure. The residue was purified by flahs column chromatography on silica gel (MeOH/DCM=1/10) to give 1-methyl-1H-indole-7-carbaldehyde (700 mg, yield 46%) as a yellow oil.

LC-MS (ESI): mass calced for: C₁₀H₉NO 159.07; m/z found, 160.10 [M+H]⁺.

### Step B: 2-methyl-N-((1-methyl-1H-indol-7-yl)methyl) propane-2-sulfinamide

To a solution of 1-methyl-1H-indole-7-carbaldehyde (700 mg, 4.397 mmol, 1.0 eq) and 2-methylpropane-2-sulfinamide (1065.83 mg, 8.794 mmol, 2.0 eq) in THF (20 mL) was added Ti (OEt)₄ (0.922 mL, 4.397 mmol, 1.0 eq) and the mixture was stirred at 70° C. for 30 min. After cooled to room temperature, NaBH4 (250 mg, 6.596 mmol, 1.5 eq) was added to above mixture at 0° C. and the mixture was stirred at 50° C. for 24 h. After cooled to room temperature, the mixture was quenched with saturated aqueous NH₄Cl solution (50 mL) and extracted with ethyl acetate (50 mL×3). The organic layer was washed with 5% aqueous KH2PO₄ solution (50 mL, pH 5 to destroy borane-complex) and brine (50 mL), dried over anhydrous sodium sulfate, filtered and concentrated under reduced pressure. The residue was dissolved in ethyl acetate (50 mL) under reflux, cooled to 22° C. while heptane (50 mL) was added in one portion. The solution was stined for 30 minutes at 22° C. while crystallisation started. Then cooled to 5° C. and stirred for 30 minutes, the solid was filtered off, washed with EtO Ac/Hep (20 mL, 1/1) and pentane (20 mL), and dried in give 2-methyl-N-[(1-methyl-1H-indol-7-yl)methyl]propane-2-sulfinamide (800 mg, yield 68.81%) as a white solid.

LC-MS (ESI): mass calced for: C14H20N2OS 264.13; m/z found, 265.1 [M+H]⁺. ¹H NMR (400 MHZ, DMSO-d₆) δ 7.46 (dd, J=7.8, 0.8 Hz, 1H), 7.22 (d, J=3.2 Hz, 1H), 7.06 (d, J=7.0 Hz, 1H), 6.94 (t, J=7.4 Hz, 1H), 6.39 (d, J=3.2 Hz, 1H), 5.64 (t, J=5.2 Hz, 1H), 4.63 (dd, J=13.8, 4.6 Hz, 1H), 4.52 (dd, J=13.8, 5.6 Hz, 1H), 4.05 (s, 3H), 1.36 (s, 9H).

### Step C: (1-methyl-1H-indol-7-yl) methanamine

To a solution of 2-methyl-N-[(1-methyl-1H-indol-7-yl)methyl]propane-2-sulfinamide (800 mg, 3.026 mmol, 1.0 eq) in dioxane (5 mL) was added hydrogen chloride (4 Nin dioxane) (3.1 mL, 12.500 mmol, 4.0 eq) at 0° C. and the resulting reaction mixture was stirred at room temperature for 2 h. The mixture was concentrated to obtain (1-methyl-1H-indol-7-yl) methanamine hydrochloride (460 mg, yield 95%) as off white solid.

LC-MS (ESI): mass calced for: Chemical Formula: C₁₀H₁₂N₂ 160.10; m/z found, 161.0 [M+H]⁺.

### Intermediate 22:2-chloroquinoline-4-carbaldehyde

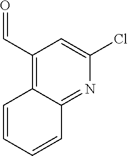 [SMILES: O=Cc1cc(Cl)nc2ccccc12]

### Step A: (2-chloroquinolin-4-yl) (pyrrolidin-1-yl) methanone

To a mixture of 2-chloroquinoline-4-carboxylic acid (1.00 g, 1.0 equiv., 4.82 mmol) in dry DMF (10 mL) was added HATU (2.20 g, 1.2 equiv., 5.78 mmol), DIEA (2.52 mL, 3.0 equiv., 14.5 mmol) and pyrrolidine (603 μL, 1.5 equiv., 7.23 mmol) at 20° C. The mixture was stirred at 20° C. for 30 min to give yellow solution. The reaction solution was poured into saturated NH₄Cl and extracted with EtOAc three times. The combined organic layers were dried over anhydrous Na₂SO₄ and concentrated under reduced pressure. The residue was purified by silica gel chromatography (100-200 mesh silica gel, 0-67% of EtOAc in PE) to afford (2-chloroquinolin-4-yl) (pyrrolidin-1-yl) methanone (1.20 g, 95.6%) as a yellow solid.

LC-MS (ESI): mass calced for: Chemical Formula: C₁₄H₁₃ClN₂O, 260.7; m/z found, 261.5 [M+H]⁺. ¹H NMR (400 MHZ, DMSO-d₆) δ 8.03 (d, J=8.4 Hz, 1H), 7.91-7.83 (m, 2H), 7.72 (dd, J=11.2, 4.0 Hz, 1H), 7.66 (s, 1H), 3.62 (t, J=7.0 Hz, 2H), 3.10 (t, J=6.7 Hz, 2H), 1.96-1.88 (m, 2H), 1.80 (p, J=6.7 Hz, 2H).

### Step B: 2-chloroquinoline-4-carbaldehyde

To a mixture of (2-chloroquinolin-4-yl) (pyrrolidin-1-yl) methanone (400 mg, 1.0 equiv., 1.53 mmol) in THF (6 mL) was added LAH (116 mg, 2.0 equiv., 3.07 mmol) under ice-water bath. The reaction was stirred at 0° C. for 5 min. The reaction solution was poured into saturated NH₄Cl and extracted with EtOAc. The combined organic layers were dried over anhydrous Na₂SO₄ and concentrated under reduced pressure. The residue was purified by silica gel chromatography (100-200 mesh silica gel, 0-30% of EtOAc in PE) to afford 2-chloroquinoline-4-carbaldehyde (150 mg, 51.0%) as a yellow solid.

LC-MS (ESI): mass calced for: Chemical Formula: C₁₀H₆ClNO, 191.0; m/z found, 192.2 [M+H]⁺. ¹H NMR (400 MHZ, DMSO-d₆) δ 10.55 (s, 1H), 8.99 (d, J=8.4 Hz, 1H), 8.21 (s, 1H), 8.16 (d, J=8.1 Hz, 1H), 8.03-7.97 (m, 1H), 7.93-7.86 (m, 1H).

### Intermediate 23:2-chloro-4-(pyrrolidin-1-ylmethyl)-5,6,7,8-tetrahydroquinoline

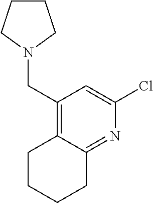 [SMILES: Clc1cc(CN2CCCC2)c2c(n1)CCCC2]

### Step A: ethyl 3-cyano-2-oxo-1,2,5,6,7,8-hexahydroquinoline-4-carboxylate

To a mixture of sodium ethanolate (26.6 g, 25% wt, 1.2 equiv., 97.8 mmol) in EtOH (30 mL) and diethyl oxalate (11.9 g, 1.0 equiv., 81.5 mmol), cyclohexanone (8.0 g, 1.0 equiv., 81.5 mmol) was added dropwise. The mixture was stirred for 3 hours at 25° C. and then 2-cyanoacetamide (6.85 g, 1.0 equiv., 81.5 mmol) was added. The reaction was stirred at 80° C. for 2 hours and then concentrated in vacuo. The residue was taken up in 150 mL boiling water and 12 mL acetic acid and stirred at 0° C. for 10 min. The precipation was occurred and the solid was isolated by filtration and dried in vacuo to afford ethyl 3-cyano-2-oxo-1,2,5,6,7,8-hexahydroquinoline-4-carboxylate (12.6 g, 62.8% yield) as a brown solid.

LC-MS (ESI): mass calced for: Chemical Formula: C₁₃H₁₄N₂O₃, 246.3; m/z found, 247.2 [M+H]⁺

### Step B: 2-oxo-1,2,5,6,7,8-hexahydroquinoline-4-carboxylic acid

A mixture of ethyl 3-cyano-2-oxo-1,2,5,6,7,8-hexahydroquinoline-4-carboxylate (12.5 g, 1 Eq, 50.8 mmol) and 36% aqueous hydrochloric acid solution (34.5 mL, 10 equiv., 508 mmol) was stirred at 115° C. overnight. Additional 2 mL of 6 M hydrochloric acid was added into the mixture and the reaction was further heated to 115° C. overnight. The hot reaction solution was poured onto ice to form precipitation and the solid was filtered. The solid was dried at 65° C. in vacuo to give 2-oxo-1,2,5,6,7,8-hexahydroquinoline-4-carboxylic acid (8.60 g, 44% yield).

LC-MS (ESI): mass calced for: Chemical Formula: C₁₀H₁₁NO₃, 193.2; m/z found, 194.1 [M+H]⁺.

### Step C: (2-chloro-5,6,7,8-tetrahydroquinolin-4-yl) (pyrrolidine-1-yl) methanone

A solution of 2-oxo-1,2,5,6,7,8-hexahydroquinoline-4-carboxylic acid (4.0 g, 1.0 equiv., 10.4 mmol) in POCl₃ (18.2 mL, 18.9 equiv., 196 mmol) was stirred at 100° C. for 2 hours. The reaction mixture is concentrated under vacuum to give the crude product. It was then added into DCM (50 mL) followed by TEA (2.89 mL, 2.0 equiv., 20.7 mmol) at 0° C. This mixture was then stirred at room temp for 2 hours. The reaction mixture was quenched with water (15 mL) and extracted with DCM (50 mL×3). The combined organic layers were washed with brine, dried over anhydrous Na₂SO₄, filtered and concentrated under reduced pressure to afford the crude product. The crude product was then purified by flash with EA/PE=48% to afford (2-chloro-5,6,7,8-tetrahydroquinolin-4-yl) (pyrrolidine-1-yl) methanone (1.00 g, 36.5% yield) as a yellow oil.

LC-MS (ESI): mass calced for: Chemical Formula: C₁₄H₁₇ClN₂O, 264.1; m/z found, 265.1 [M+H]⁺. ¹H NMR (400 MHZ, DMSO-d₆) δ 7.23 (s, 1H), 3.45 (t, J=6.7 Hz, 2H), 3.08 (t, J=6.3 Hz, 2H), 2.81 (t, J=6.3 Hz, 2H), 2.55 (t, J=6.0 Hz, 2H), 1.91-1.68 (m, 9H).

### Step D: 2-chloro-4-(pyrrolidin-1-ylmethyl)-5,6,7,8-tetrahydroquinoline

To a solution of (2-chloro-5,6,7,8-tetrahydroquinolin-4-yl) (pyrrolidin-1-yl) methanone (500 mg, 1.0 equiv., 1.89 mmol) in THF (10 mL), was added LAH (143 mg, 2.0 equiv., 3.78 mmol) at 0° C. The reaction mixture was stirred at 0° C. for 10 min. The reaction solution was added into saturated NH₄Cl at 0° C. The mixed solution was filtered and and the filtrate was partitioned betwen water (3 mL) and EA (5 mL×3). The organic layers were washed with brine, dried with anhydrous Na₂SO₄ and purified by Pre-TLC(PE:EA=1:1) to give 2-chloro-4-(pyrrolidin-1-ylmethyl)-5,6,7,8-tetrahydroquinoline (40 mg, 8.45% yield) as a yellow solid.

LC-MS (ESI): mass calced for: Chemical Formula: C₁₄H₁₉ClN, 193.2; m/z found, 251 [M+H]⁺.

### Intermediate 24:2-chloro-6,7-dihydro-5H-cyclopenta[b]pyridine-4-carbaldehyde

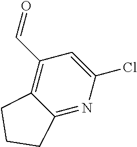 [SMILES: O=Cc1cc(Cl)nc2c1CCC2]

### Step A: 3-cyano-2-oxo-2,5,6,7-tetrahydro-1H-cyclopenta[b]pyridine-4-carboxylate

To a mixture of sodium ethoxide (20%, 27.9 mL, 1.2 equiv., 71.3 mmol) solution in ethanol and cyclopentanone (5.00 g, 1.0 equiv., 59.4 mmol), diethyl oxalate (8.69 g, 1.0 equiv., 59.4 mmol) was added dropwise. The mixture was stirred at 25° C. for 3 hours. Then 2-cyanoacetamide (5.00 g, 1.0 equiv., 59.4 mmol) was added. The reaction was stirred at 80° C. for 2 hours. The reaction mixture was concentrated in vacuo. The residue was taken up in 250 mL boiling water and 20 mL acetic acid at 0° C. The solid was precipated and isolated by filtration and the collected solid was dried in vacuo to give ethyl 3-cyano-2-oxo-2,5,6,7-tetrahydro-1H-cyclopenta[b]pyridine-4-carboxylate (4.90 g, 35.5% yield) as a green solid.

LC-MS (ESI): mass calced for: Chemical Formula: C₁₂H₁₂N₂O₃, 232.2; m/z found, 233.1 [M+1]⁺. ¹H NMR (400 MHZ, DMSO-d₆) δ 13.22 (s, 1H), 4.38 (q, J=7.1 Hz, 2H), 2.87 (t, J=7.7 Hz, 2H), 2.79 (t, J=7.3 Hz, 2H), 2.09-1.99 (m, 2H), 1.32 (t, J=7.1 Hz, 3H).

### Step B: 2-oxo-2,5,6,7-tetrahydro-1H-cyclopenta[b]pyridine-4-carboxylic acid

To a solution of ethyl 3-cyano-2-oxo-2,5,6,7-tetrahydro-1H-cyclopenta[b]pyridine-4-carboxylate (4.90 g, 1.0 equiv., 21.1 mmol) in 6 N HCl in water (35.2 mL, 211 mmol). The mixture was stirred at 115° C. for 16 hours. The hot reaction solution was poured into ice to precipate the solid. The solid was then filtered. The filter cake was dried at 45° C. in vacuo to give 2-oxo-2,5,6,7-tetrahydro-1H-cyclopenta[b]pyridine-4-carboxylic acid (3.00 g, 79.4%) as a red solid.

LC-MS (ESI): mass calced for: Chemical Formula: C₉H₉NO₃, 179.2; m/z found, 180.2 (M+H)+. ¹H NMR (400 MHZ, DMSO-d₆) δ 6.64 (s, 1H), 2.89 (t, J=7.4 Hz, 2H), 2.76 (t, J=7.7 Hz, 2H), 2.05-1.95 (m, 2H).

### Step C: methyl 2-chloro-6,7-dihydro-5H-cyclopenta[b]pyridine-4-carboxylate

To a solution of 2-oxo-2,5,6,7-tetrahydro-1H-cyclopenta[b]pyridine-4-carboxylic acid (600 mg, 1.0 equiv., 3.35 mmol) in POCl₃ (6.00 mL, 64.4 mmol). The mixture was stirred at 90° C. for 16 hours. The reaction mixture is concentrated under vacuum to give crude product of 2-chloro-6,7-dihydro-5H-cyclopenta[b]pyridine-4-carbonyl chloride (600 mg, 82.9%) as a brown oil.

To a solution of 2-chloro-6,7-dihydro-5H-cyclopenta[b]pyridine-4-carbonyl chloride (600 mg, 1.85 mmol) in DCM (3.0 mL), was added with methanol (600 mL, 18.5 mmol) at −40° C. This mixture was stirred at room temp for 20 min. The reaction mixture was quenched with water (15 mL) and extracted with DCM (15 mL×3). The combined organic layer was washed with brine solution and dried over anhydrous Na₂SO₄, filtered and concentrated under reduced pressure to afford the crude product. The crude product was then purified by flash with EA/PE=20% to afford methyl 2-chloro-6,7-dihydro-5H-cyclopenta[b]pyridine-4-carboxylate (300 mg, 76.6%) as a yellow solid.

LC-MS (ESI): mass calced for: Chemical Formula: C₁₀H₁₀ClNO₂, 211.6; m/z found, 198.4 (M-19)+. ¹H NMR (400 MHZ, DMSO-d₆) δ 7.57 (s, 1H), 3.88 (s, 3H), 3.16 (t, J=7.6 Hz, 2H), 2.97 (t, J=7.8 Hz, 2H), 2.09 (p, J=7.7 Hz, 2H).

### Step D: (2-chloro-6,7-dihydro-5H-cyclopenta[b]pyridin-4-yl) methanol

To a solution of methyl 2-chloro-6,7-dihydro-5H-cyclopenta[b]pyridine-4-carboxylate (300 mg, 1.0 equiv., 1.42 mmol) in THF (5.0 mL), was added LiAlH4 (53.8 mg, 1.0 equiv., 1.42 mmol) at 0° C. The reaction mixture was stirred at 0° C. for 10 min. Na₂SO₄·10H₂O was added into the mixture at 0° C. The mixed solution was filtered and and the filtrate was partitioned between water (3 mL) and EA (5 mL×3). The organic layer was washed with brine, dried with anhydrous Na₂SO₄ and concentrated in vacuum to give (2-chloro-6,7-dihydro-5H-cyclopenta[b]pyridin-4-yl) methanol (250 mg, 96.0%) as a yellow solid.

LC-MS (ESI): mass calced for: Chemical Formula: C₉H₁₀ClNO, 183.6; m/z found, 184.5 [M+1]*.

### Step E: 2-chloro-6,7-dihydro-5H-cyclopenta[b]pyridine-4-carbaldehyde

To a solution of (2-chloro-6,7-dihydro-5H-cyclopenta[b]pyridin-4-yl) methanol (250 mg, 1 equiv., 1.36 mmol) in DCM (10 mL) was added with DMP (866 mg, 1.5 equiv., 2.04 mmol) at 0° C. The reaction mixture was stirred at room temp for 1.5 hours. The reaction mixture was quenched by dropping aquesous staruated NaHCO₃ (20 mL). The mixed solution was filtered through a apd of Celite and and the filtrate was extracted with DCM (15 mL×3). The organic layer was washed with brine, dried with anhydrous Na₂SO₄ and concentrated in vacuum. The crude product was then purified by Prep-TLC with PE/EA=2.5:1 to give 2-chloro-6,7-dihydro-5H-cyclopenta[b]pyridine-4-carbaldehyde (150 mg, 60.7% yield) as a yellow solid.

LC-MS (ESI): mass calcd. for C₉H₈ClNO, 181.0; m/z found, 182.1 [M+1]⁺.

### Intermediate 25:5-chloro-3-(isopropylamino)-1-((2-(trimethylsilyl)ethoxy)methyl)-1H-pyrazolo[4,3-b]pyridine-7-carbaldehyde

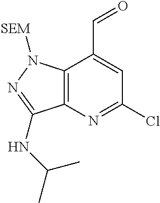 [SMILES: CC(C)Nc1nn(COCC[Si](C)(C)C)c2c(C=O)cc(Cl)nc12]

### Step A: 4-bromo-6-chloro-2-methylpyridin-3-amine

To a solution of 6-chloro-2-methylpyridin-3-amine (40 g, 0.28 mol, 1.0 eq) and AcOH (30.4 mL, 0.53 mol, 1.9 eq) in MeOH (400 mL) was added dropwise Br2 (26 mL, 0.504 mol, 1.8 eq) at 0° C. and the mixture was stirred at 0° C. for 6 h. After evaporation, the reaction mixture was diluted with EA (1 L), washed with saturated aqueous sodium thiosulfate solution (500 mL×2), saturated aqueous NaHCO₃ solution (500 mL×2) and brine (500 mL), dried over anhydrous sodium sulfate, filtered and concentrated under reduced pressure. The residue was purified by flash column chromatography on silica gel (PE/EA=10/1 v/v) to afford 4-bromo-6-chloro-2-methylpyridin-3-amine (55 g, yield 90%) as a yellow solid. LC-MS (ESI): mass calcd. for C₆H₆BrClN₂, 219.94; m/z found, 221.2 [M+H]⁺. ¹H NMR (400 MHZ, DMSO-d6) δ 7.41 (s, 1H), 5.39 (s, 2H), 2.33 (s, 3H).

### Step B: 7-bromo-5-chloro-1H-pyrazolo[4,3-b]pyridine

To a solution of 4-bromo-6-chloro-2-methylpyridin-3-amine (25 g, 113 mmol, 1.0 eq) in toluene (625 mL) were added potassium acetate (22.2 g, 226 mmol, 2.0 eq) and AcOH (210 mL) at 0° C. Then isoamyl nitrite (19.8 g, 22.7 mL, 169 mmol, 1.5 eq) was added dropwise to above mixture at 0° C. The resulting mixture was stirred at 0° C. for 6 h. After evaporation, the mixture was poured into ice-water (300 mL), adjusted to pH 7˜8 with solid NaHCO₃, and extracted with EA (300 mL×3). The organic layer was washed with water (300 mL) and brine (300 mL), dried over anhydrous Na₂SO₄, filtered and concentrated under reduced pressure. The residue was purified with flash column chromatography on silica gel (ethyl acetate in petroleum ether, from 2% to 15% v/v) to give 7-bromo-5-chloro-1H-pyrazolo[4,3-b]pyridine (8.4 g, yield 32%) as a yellow solid. LC-MS (ESI): mass calcd. for C₆H₃BrClN₃, 230.92; m/z found, 232.2 [M+H]⁺. ¹H NMR (400 MHZ, DMSO-d₆) δ 14.14 (s, 1H), 8.43 (s, 1H), 7.87 (s, 1H).

### Step C: 7-bromo-5-chloro-3-nitro-1H-pyrazolo[4,3-b]pyridine

To a solution of 7-bromo-5-chloro-1H-pyrazolo[4,3-b]pyridine (15.0 g, 64.5 mmol, 1.0 eq) in con. H₂SO₄ (48 mL) was added dropwise a solution of con. H₂SO₄/con. HNO₃ (96 mL, 1/1 v/v) (con. HNO₃ was added to con. H₂SO₄ at 0° C.) at 0° C. Then the mixture was stirred at 110° C. for 2 h. After cooled to room temperature, the mixture was poured into ice-water (300 mL) and stirred for 0.5 h. The mixture was extracted with EA (200 mL×3). The organic layer was washed with H₂O (300 mL×4) and brine (300 mL), dried over anhydrous sodium sulfate, and filtered. The filtration was concentrated under reduced pressure to get 7-bromo-5-chloro-3-nitro-1H-pyrazolo[4,3-b]pyridine (17.9 g, yield 100%) as a yellow solid. LC-MS (ESI): mass calcd. for C₆H₂BrClN₄O₂, 275.90; m/z found, 276.9 [M+H]⁺. ¹H NMR (400 MHZ, DMSO-d₆) δ 13.35 (s, 1H), 8.13 (s, 1H).

### Step D: 7-bromo-5-chloro-3-nitro-1-((2-(trimethylsilyl)ethoxy)methyl)-1H-pyrazolo[4,3-b]pyridine (4a) and 7-bromo-5-chloro-3-nitro-2-((2-(trimethylsilyl)ethoxy)methyl)-2H-pyrazolo[4,3-b]pyridine

To a solution of 7-bromo-5-chloro-3-nitro-1H-pyrazolo[4,3-b]pyridine (35.8 g, 129 mmol, 1.0 eq) in anhydrous DMF (500 mL) was added sodium hydride (60% suspend in oil) (9.29 g, 232 mmol, 1.8 eq) in portions at 0° C. and the mixture was stirred at 0° C. for 30 min. Then 2-(trimethylsilyl)ethoxymethyl chloride (25.8 g, 27.4 mL, 155 mmol, 1.2 eq) was added dropwise to above mixture and the resulting mixture was stirred at 0° C. for 30 min. The mixture was quenched with saturated aqueous NH₄Cl solution (1000 mL) at 0° C. and extracted with EA (800 mL×3). The organic layer was washed with H₂O (500 mL×4) and brine (500 mL), dried over anhydrous sodium sulfate, filtered and concentrated under reduced pressure. The residue was purified with flash column chromatography on silica gel (ethyl acetate in petroleum ether, from 0% to 10%) to give 7-bromo-5-chloro-3-nitro-1-((2-(trimethylsilyl)ethoxy)methyl)-1H-pyrazolo[4,3-b]pyridine (28.2 g, yield 54%) as a yellow solid and 7-bromo-5-chloro-3-nitro-2-((2-(trimethylsilyl)ethoxy)methyl)-2H-pyrazolo[4,3-b]pyridine (14.8 g, yield 28%) as a yellow solid.

7-bromo-5-chloro-3-nitro-1-((2-(trimethylsilyl)ethoxy)methyl)-1H-pyrazolo[4,3-b]pyridine: LC-MS (ESI): mass calcd. for C₁₂H₁₆BrClN₄O₃Si, 405.99; m/z found, 407.0 [M+H]⁺. ¹H NMR (400 MHZ, DMSO-d₆) δ 8.35 (s, 1H), 6.16 (s, 2H), 3.71 (t, J=7.8 Hz, 2H), 0.93 (t, J=7.8 Hz, 2H), −0.00 (s, 9H).

7-bromo-5-chloro-3-nitro-2-((2-(trimethylsilyl)ethoxy)methyl)-2H-pyrazolo[4,3-b]pyridine: LC-MS (ESI): mass calcd. for C₁₂H₁₆BrClN₄O₃Si, 405.99; m/z found, 407.0 [M+H]⁺. ¹H NMR (400 MHZ, DMSO-d₆) δ 8.24 (s, 1H), 6.26 (s, 2H), 3.77 (dd, J=16.7, 8.9 Hz, 2H), 0.94 (t, J=8.1 Hz, 2H), −0.00 (s, 9H).

### Step E: 7-bromo-5-chloro-1-((2-(trimethylsilyl)ethoxy)methyl)-1H-pyrazolo[4,3-b]pyridin-3-amine

To a solution of 7-bromo-5-chloro-3-nitro-1-((2-(trimethylsilyl)ethoxy)methyl)-1H-pyrazolo[4,3-b]pyridine (28.0 g, 68.7 mmol, 1.0 eq) and Ammonium chloride (18.4 g, 343 mmol, 5.0 eq) in EtOH (400 mL), THF (400 mL), and Water (200 mL) was added Iron powder (19.2 g, 343 mmol, 5.0 eq) at room temperature. The mixture was stirred at 70° C. for 2 h. After cooled to room temperature, the mixture was filtered and the cake was washed with EA (200 mL×3). The filtrate was concentrated under reduced pressure and the residue was diluted with EA (1200 mL). The organic layer was washed with water (500 mL) and brine (500 mL), dried over anhydrous Na₂SO₄, filtered and concentrated under reduced pressure to give 7-bromo-5-chloro-1-((2-(trimethylsilyl)ethoxy)methyl)-1H-pyrazolo[4,3-b]pyridin-3-amine (26.0 g, yield 100%) as a yellow solid, which was directly used in next step without further purification. LC-MS (ESI): mass calcd. for C₁₂H₁₈BrClN₄OSi, 376.01; m/z found, 377.2 [M+H]⁺. ¹H NMR (400 MHZ, DMSO-d₆) δ 7.96 (s, 1H), 6.02 (s, 2H), 5.78 (s, 2H), 3.64 (t, J=7.8 Hz, 2H), 0.89 (t, J=7.8 Hz, 2H), 0.00 (s, 9H).

### Step F: 7-bromo-5-chloro-N-isopropyl-1-((2-(trimethylsilyl)ethoxy)methyl)-1H-pyrazolo[4,3-b]pyridin-3-amine

To a solution of 7-bromo-5-chloro-1-((2-(trimethylsilyl)ethoxy)methyl)-1H-pyrazolo[4,3-b]pyridin-3-amine (5.00 g, 13.2 mmol, 1.0 eq) in Acetone (150 mL) was added Acetic acid (5.23 g, 5.00 mL, 87.0 mmol, 6.57 eq) at room temperature and the mixture was stirred at 50° C. for 4 h. Then Sodium cyanoborohydride (4.16 g, 66.2 mmol, 5.0 eq) was added to above mixture in portions and the reaction mixture was stirred at 50° C. overnight. After evaporation, the residue was diluted with EA (200 mL), washed with saturated aqueous NaHCO₃ solution (200 mL×3), water (200 mL) and brine (200 mL), dried over anhydrous Na₂SO₄, filtered and concentrated under reduced pressure to give a crude product. The crude product was purified by flash column chromatography on silica gel (PE/EA=15/1 to 10/1 v/v) to give 7-bromo-5-chloro-N-isopropyl-1-((2-(trimethylsilyl)ethoxy)methyl)-1H-pyrazolo[4,3-b]pyridin-3-amine (3.20 g, yield 58%) as a yellow solid. LC-MS (ESI): mass calcd. for C₁₅H₂₄BrClN₄OSi, 418.06; m/z found, 419.0 [M+H]⁺. ¹H NMR (400 MHZ, DMSO-d₆) δ 7.98 (s, 1H), 6.32 (d, J=7.6 Hz, 1H), 5.83 (s, 2H), 4.02-3.98 (m, 1H), 3.67 (t, J=7.6 Hz, 2H), 1.35 (d, J=6.0 Hz, 6H), 0.90 (t, J=7.64 Hz, 2H), −0.00 (s, 9H).

### Step G: 5-chloro-N-isopropyl-1-((2-(trimethylsilyl)ethoxy)methyl)-7-vinyl-1H-pyrazolo[4,3-b]pyridin-3-amine

To a solution of 7-bromo-5-chloro-N-isopropyl-1-((2-(trimethylsilyl)ethoxy)methyl)-1H-pyrazolo[4,3-b]pyridin-3-amine (2.30 g, 5.48 mmol, 1.0 eq) and

Potassium Vinyltrifluoroborate (1.10 g, 8.22 mmol, 1.5 eq), and Potassium phosphate tribasic (3.49 g, 16.4 mmol, 3.0 eq) in 1,4-Dioxane (50.00 mL) and Water (5.00 mL) was added Pd(dppf)Cl₂ (401 mg, 548 μmol, 0.1 eq) at room temperature. The reaction mixture was stirred at 85° C. for 2.5-3 h. The reaction mixture is cooled to room temperature and concentrated under reduced pressure. The residue was diluted with EA (100 mL), washed with water (100 mL) and brine (100 mL). The organic layer was dried over sodium sulfate, filtered and concentrated under reduced pressure to give a crude product. The crude product was purified by flash column chromatography on silica gel (ethyl acetate in petroleum ether, 6% v/v) to afford 5-chloro-N-isopropyl-1-((2-(trimethylsilyl)ethoxy)methyl)-7-vinyl-1H-pyrazolo[4,3-b]pyridin-3-amine (1.63 g, yield 81%) as a red oil. LC-MS (ESI): mass calcd. for C₁₇H₂₇ClN4OSi, 366.16; m/z found, 367.2 [M+H]⁺. ¹H NMR (400 MHZ, DMSO-d₆) δ 7.66 (s, 1H), 7.37 (dd, J=17.4, 11.2 Hz, 1H), 6.32 (d, J=17.4 Hz, 1H), 6.09 (d, J=8.0 Hz, 1H), 5.83 (d, J=11.8 Hz, 1H), 5.66 (s, 2H), 4.05-3.96 (m, 1H), 3.68-3.58 (m, 2H), 1.35 (d, J=6.4 Hz, 6H), 0.96-0.84 (m, 2H), 0.00 (s, 9H).

### Step H: 1-(5-chloro-3-(isopropylamino)-1-((2-(trimethylsilyl)ethoxy)methyl)-1H-pyrazolo[4,3-b]pyridin-7-yl) ethane-1,2-diol

To a solution of 5-chloro-N-isopropyl-1-((2-(trimethylsilyl)ethoxy)methyl)-7-vinyl-1H-pyrazolo[4,3-b]pyridin-3-amine (6.10 g, 16.6 mmol, 1.0 eq) in Acetone (120.00 mL) and water (60.0 mL) were added NMO (3.89 g, 33.2 mmol, 2.0 eq) and potassium osmate (VI) dihydrate (612 mg, 1.66 mmol, 0.1 eq). The reaction was stirred at 15° C. for 16 h. After filtration, the filtrate was evaporated under reduced pressure. The residue was diluted with EA (250 mL), washed with water (150 mL) and brine (150 mL), dried over anhydrous MgSO₄, filtered and concentrated under reduced pressure to give a crude product. The crude product was purified by flash column chromatography on silica gel (ethyl acetate in petroleum ether, 36% v/v) to afford 1-(5-chloro-3-(isopropylamino)-1-((2-(trimethylsilyl)ethoxy)methyl)-1H-pyrazolo[4,3-b]pyridin-7-yl) ethane-1,2-diol (4.78 g, yield 72%) as a black oil. LC-MS (ESI): mass calcd. for C₁₇H₂₉ClN₄O₃Si, 400.17; m/z found, 401.1 [M+H]⁺. ¹H NMR (400 MHZ, DMSO-d₆) δ 7.53 (s, 1H), 6.01 (d, J=8.0 Hz, 1H), 5.93 (d, J=5.0 Hz, 1H), 5.77 (d, J=11.8 Hz, 1H), 5.59 (d, J=11.8 Hz, 1H), 5.23 (q, J=5.6 Hz, 1H), 5.13 (t, J=5.6 Hz, 1H), 3.98 (dq, J=13.0, 6.4 Hz, 1H), 3.72 (t, J=5.6 Hz, 2H), 3.67-3.51 (m, 2H), 1.32 (d, J=6.4 Hz, 6H), 0.95-0.82 (m, 2H), 0.00 (s, 9H).

### Step I: 5-chloro-3-(isopropylamino)-1-((2-(trimethylsilyl)ethoxy)methyl)-1H-pyrazolo[4,3-b]pyridine-7-carbaldehyde

To a solution of 1-(5-chloro-3-(isopropylamino)-1-((2-(trimethylsilyl)ethoxy)methyl)-1H-pyrazolo[4,3-b]pyridin-7-yl) ethane-1,2-diol (2.10 g, 5.24 mmol, 1.0 eq) in THF (20 mL) and water (10 mL) was added NaIO4 (2.24 g, 10.5 mmol, 2.0 eq) at 0° C. The reaction mixture was stirred at 0° C. for 1 h. The reaction mixture was quenched by adding saturated aqueous sodium thiosulfate solution (30 mL) and extracted with EA (40 mL×3). The combined organic layers were washed water (50 mL) and brine (50 mL), dried over anhydrous Na₂SO₄, filtered and concentrated under reduced pressure. The crude product was purified by flash column chromatography on silica gel (ethyl acetate in petroleum ether, 15% v/v) to afford 5-chloro-3-(isopropylamino)-1-((2-(trimethylsilyl)ethoxy)methyl)-1H-pyrazolo[4,3-b]pyridine-7-carbaldehyde (1.70 g, yield 88%) as a red solid. LC-MS (ESI): mass calcd. for C₁₆H₂₅ClN₄O₂Si, 368.14; m/z found, 387.2 [M+18+H]⁺. ¹H NMR (400 MHZ, DMSO-d₆) δ 10.42 (s, 1H), 8.08 (s, 1H), 6.46 (d, J=8.0 Hz, 1H), 5.94 (s, 2H), 4.12-3.98 (m, 1H), 3.59 (t, J=8.0 Hz, 2H), 1.39 (d, J=6.4 Hz, 6H), 0.88 (t, J=8.0 Hz, 2H), 0.00 (s, 9H).

### Intermediate 26:3-amino-5-chloro-2-((2-(trimethylsilyl)ethoxy)methyl)-2H-pyrazolo[4,3-b]pyridine-7-carbaldehyde

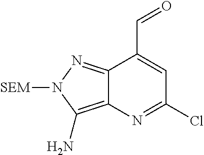 [SMILES: C[Si](C)(C)CCOCn1nc2c(C=O)cc(Cl)nc2c1N]

### Step A: 7-bromo-5-chloro-2-((2-(trimethylsilyl)ethoxy)methyl)-2H-pyrazolo[4,3-b]pyridin-3-amine

To a solution of 7-bromo-5-chloro-3-nitro-2-((2-(trimethylsilyl)ethoxy)methyl)-2H-pyrazolo[4,3-b]pyridine (14.8 g, 36.3 mmol, 1.0 eq) and Ammonium chloride (9.71 g, 181 mmol, 5.0 eq) in EtOH (200.0 mL), THF (200.0 mL), and water (100.0 mL) was added Iron powder (10.1 g, 181 mmol, 5.0 eq) at room temperature. The mixture was stirred at 70° C. for 1 h. After cooled to room temperature, the mixture was filtered and the cake was washed with EA (150 mL×3). The filtrate was concentrated under reduced pressure and the residue was diluted with EA (500 mL). The organic layer was washed with water (200 mL) and brine (200 mL), dried over anhydrous Na₂SO₄, filtered and concentrated under reduced pressure to give 7-bromo-5-chloro-2-((2-(trimethylsilyl)ethoxy)methyl)-2H-pyrazolo[4,3-b]pyridin-3-amine (13.7 g, yield 100%) as a yellow solid, which was used directly in next step without further purification. LC-MS (ESI): mass calcd. for C₁₂H₁₈BrClN₄OSi, 376.01; m/z found, 377.0 [M+H]⁺. ¹H NMR (400 MHZ, DMSO-d₆) δ 7.63 (s, 1H), 6.89 (s, 2H), 5.63 (s, 2H), 3.68 (t, J=7.8 Hz, 2H), 0.91 (t, J=7.8 Hz, 2H),-0.00 (s, 9H).

### Step B: methyl 3-amino-5-chloro-2-((2-(trimethylsilyl)ethoxy)methyl)-2H-pyrazolo[4,3-b]pyridine-7-carboxylate

To a solution of 7-bromo-5-chloro-2-((2-(trimethylsilyl)ethoxy)methyl)-2H-pyrazolo[4,3-b]pyridin-3-amine (5.00 g, 13.2 mmol, 1.0 eq) and triethylamine (4.02 g, 5.52 mL, 39.7 mmol, 3.0 eq) in DMF (60.0 mL) and MeOH (60.0 mL) was added Pd (dppf) Cl₂ (969 mg, 1.32 mmol, 0.1 eq). The mixture was stirred under CO (balloon) at 70° C. for 6 h. After cooled to room temperature, MeOH was removed under reduced pressure and the solution was extracted with EA (100 ml×3). The organic layer was washed with brine (80 mL×4), dried over anhydrous sodium sulfate, filtered and concentrated under reduced pressure. The residue was purified by flash column chromatography on silica gel (EA/PE=1/1 v/v) to give methyl 3-amino-5-chloro-2-((2-(trimethylsilyl)ethoxy)methyl)-2H-pyrazolo[4,3-b]pyridine-7-carboxylate (3.3 g, yield 72%) as a red solid. LC-MS (ESI): mass calcd. for C₁₄H₂₁ClN₄O₃Si, 356.11; m/z found, 357.1 [M+H]⁺.

¹H NMR (400 MHZ, DMSO-d₆) δ 7.61 (s, 1H), 6.98 (s, 2H), 5.64 (s, 2H), 3.95 (s, 3H), 3.68 (t, J=7.8 Hz, 2H), 0.91 (t, J=7.8 Hz, 2H),-0.00 (s, 9H).

### Step C: (3-amino-5-chloro-2-((2-(trimethylsilyl)ethoxy)methyl)-2H-pyrazolo[4,3-b]pyridin-7-yl) methanol

To a solution of methyl 3-amino-5-chloro-2-((2-(trimethylsilyl)ethoxy)methyl)-2H-pyrazolo[4,3-b]pyridine-7-carboxylate (1.00 g, 2.80 mmol, 1.0 eq) in THF (40.0 mL) was added Aluminum lithium hydride (213 mg, 5.60 mmol, 2.0 eq) at 0° C. The reaction mixture was stirred at 0° C. for 30 min. The reaction mixture was quenched with Na₂SO₄·10H₂O and stirred for 30 min. After filtration, the filtrate was diluted with water (15 mL) and extracted with ethyl acetate (20 mL×3). The combined organic extracts were washed with water (20 mL) and brine (20 mL), dried over anhydrous sodium sulfate, filtered and concentrated under reduced pressure to give (3-amino-5-chloro-2-((2-(trimethylsilyl)ethoxy)methyl)-2H-pyrazolo[4,3-b]pyridin-7-yl) methanol (600 mg, yield 65%) as a yellow solid, which was used directly in next step without further purification. LC-MS (ESI): mass calcd. for C₁₃H₂₁ClN₄O₂Si, 328.11; m/z found, 329.1 [M+H]⁺. ¹H NMR (400 MHZ, DMSO-d₆) δ 7.15 (s, 1H), 6.61 (s, 2H), 5.59 (s, 2H), 5.56 (d, J=5.6 Hz, 1H), 4.88-4.76 (m, 2H), 3.72-3.63 (m, 2H), 0.95-0.87 (m, 2H),-0.01 (s, 9H).

### Step D: 3-amino-5-chloro-2-((2-(trimethylsilyl)ethoxy)methyl)-2H-pyrazolo[4,3-b]pyridine-7-carbaldehyde

To a solution of (3-amino-5-chloro-2-((2-(trimethylsilyl)ethoxy)methyl)-2H-pyrazolo[4,3-b]pyridin-7-yl) methanol (315 mg, 958 μmol, 1.0 eq) in DMSO (20.0 mL) was added IBX (536 mg, 1.92 mmol, 2.0 eq) at room temperature (10° C.). The reaction mixture was stirred at 27° C. for 1 h. The reaction mixture was diluted with water (40 mL) and extracted with ethyl acetate (40 mL×3). The combined organic extracts were washed with saturated aqueous Na₂S₂O₃ solution (40 mL×2), saturated aqueous NaHCO₃ solution (40 mL) and brine (40 mL), dried over anhydrous sodium sulfate, filtered and concentrated under reduced pressure. The crude product was purified by Prep-TLC(PE/EA=6/1) to provide 3-amino-5-chloro-2-((2-(trimethylsilyl)ethoxy)methyl)-2H-pyrazolo[4,3-b]pyridine-7-carbaldehyde (50.0 mg, yield 16%) as a red solid. LC-MS (ESI): mass calcd. for C₁₃H₁₉ClN₄O₂Si, 326.10; m/z found, 327.1 [M+H]⁺. ¹HNMR (400 MHZ, DMSO-d₆) δ 10.34 (s, 2H), 7.70 (s, 1H), 7.10 (s, 2H), 5.65 (s, 2H), 3.70-3.61 (m, 2H), 0.94-0.85 (m, 2H),-0.00 (s, 9H).

### Intermediate 27:3-amino-5-chloro-1-((2-(trimethylsilyl)ethoxy)methyl)-1H-pyrazolo[4,3-b]pyridine-7-carbaldehyde

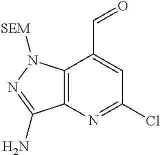 [SMILES: C[Si](C)(C)CCOCn1nc(N)c2nc(Cl)cc(C=O)c21]

### Step A: methyl 3-amino-5-chloro-1-((2-(trimethylsilyl)ethoxy)methyl)-1H-pyrazolo[4,3-b]pyridine-7-carboxylate

To a stirred solution of 7-bromo-5-chloro-1-((2-(trimethylsilyl)ethoxy)methyl)-1H-pyrazolo[4,3-b]pyridin-3-amine (18.0 g, 47.7 mmol, 1.0 eq) in DMF (50.0 mL) and MeOH (50.0 mL) were added Pd(dppf)Cl₂ (3.49 g, 4.77 mmol, 0.1 eq) and TEA (14.5 g, 19.9 mL, 143 mmol, 3.0 eq). The reaction mixture was stirred under CO atmosphere (balloon) at 70° C. for 16 h. After cooled to room temperature, the mixture was filtered and the filtrated was diluted with EtOAc (600 mL). The organic layer was washed with brine (200 mL×3), dried over anhydrous Na₂SO₄, filtered and concentrated under reduced pressure. The residue was purified by flash column chromatography on silica gel (PE/EtOAc=1/1 v/v) to give methyl 3-amino-5-chloro-1-((2-(trimethylsilyl)ethoxy)methyl)-1H-pyrazolo[4,3-b]pyridine-7-carboxylate (14.0 g, yield 82%, Purity 50%) as a brown oil. LC-MS (ESI): mass calcd. for C₁₄H₂₁ClN₄O₃Si, 356.11; m/z found, 357.1 [M+H]⁺.

### Step B: (3-amino-5-chloro-1-((2-(trimethylsilyl)ethoxy)methyl)-1H-pyrazolo[4,3-b]pyridin-7-yl) methanol

To a solution of methyl 3-amino-5-chloro-1-((2-(trimethylsilyl)ethoxy)methyl)-1H-pyrazolo[4,3-b]pyridine-7-carboxylate (4.00 g, 11.2 mmol, 1.0 eq) in THF (20.0 mL) at 0° C. was added LiAlH4 (638 mg, 16.8 mmol, 1.5 eq) in portions. The resulting mixture was stirred at 0° C. for 1 h. The reaction was quenched with sodium sulfate decahydrate and filtered. The filtrate was concentrated under reduced pressure and purified by flash column chromatography on silica gel (PE/EtOAc v/v)═1/1 to give (3-amino-5-chloro-1-((2-(trimethylsilyl)ethoxy)methyl)-1H-pyrazolo[4,3-b]pyridin-7-yl) methanol (1.40 g, yield 38%) as a light yellow solid. LC-MS (ESI): mass calcd. for C₁₃H₂₁ClN₄O₂Si, 328.11; m/z found, 329.1 [M+H]⁺. ¹H NMR (400 MHZ, CDCl₃) δ 7.35 (s, 1H), 5.62 (s, 2H), 5.07 (s, 2H), 4.69 (s, 2H), 4.63-4.44 (m, 1H), 3.60-3.52 (m, 2H), 0.94-0.84 (m, 2H),-0.00 (s, 9H).

### Step C: 3-amino-5-chloro-1-((2-(trimethylsilyl)ethoxy)methyl)-1H-pyrazolo[4,3-b]pyridine-7-carbaldehyde

To a stirred mixture of (3-amino-5-chloro-1-((2-(trimethylsilyl)ethoxy)methyl)-1H-pyrazolo[4,3-b]pyridin-7-yl) methanol (1.40 g, 4.26 mmol, 1.0 eq) in DMSO (20.0 mL) was added IBX (1.79 g, 6.39 mmol, 1.5 eq). The resulting mixture was stirred at 25° C. for 2 h. The reaction mixture was quenched with saturated aqueous Na₂SO₃ solution (30 mL) and extracted with EtOAc (80 mL×3). The combined organic layers were washed with brine (100 mL×4), dried over anhydrous Na₂SO₄, filtered and concentrated under reduced pressure. The crude product was purified by flash column chromatography on silica gel (ethyl acetate in petroleum ether, 10% v/v) to give 3-amino-5-chloro-1-((2-(trimethylsilyl)ethoxy)methyl)-1H-pyrazolo[4,3-b]pyridine-7-carbaldehyde (900 mg, yield 64%) as a red solid. LC-MS (ESI): mass calcd. for C₁₃H₁₉ClN₄O₂Si, 326.10; m/z found, 327.1 [M+H]⁺.

### Intermediate 28:3-(3-methyl-1-oxo-5-(4,4,5,5-tetramethyl-1,3,2-dioxaborolan-2-yl) isoindolin-2-yl) piperidine-2,6-dione

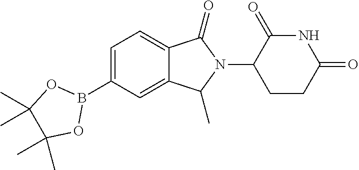 [SMILES: CC1c2cc(B3OC(C)(C)C(C)(C)O3)ccc2C(=O)N1C1CCC(=O)NC1=O]

### Step A: methyl 4-bromo-2-ethylbenzoate

To the solution of 4-bromo-2-ethylbenzoic acid (5.0 g, 21.8 mmol, 1.0 eq) in DMF (25.0 mL) were added potassium carbonate (6.03 g, 43.7 mmol, 2.0 eq) and iodomethane (4.65 g, 2.04 mL, 32.7 mmol, 1.5 eq). The mixture was stirred at room temperature for 16 h. After filtered, the cake was washed with EA (50 mL). The filtrate was diluted with EA (200 mL), washed with brine (100 mL×4), dried over anhydrous Na₂SO₄, filtered and concentrated under reduced pressure to give methyl 4-bromo-2-ethylbenzoate (4.4 g, yield 83%) as a yellow solid. LC-MS (ESI): mass calcd. for C₁₀H₁₁BrO₂, 241.99; m/z found, no mass [M+H]⁺. ¹H NMR (400 MHZ, DMSO-d₆) δ 7.71 (d, J=8.4 Hz, 1H), 7.59 (s, 1H), 7.53 (d, J=8.4 Hz, 1H), 3.84 (s, 3H), 2.89 (q, J=7.4 Hz, 2H), 1.15 (t, J=7.4 Hz, 3H)

### Step B: methyl 4-bromo-2-(1-bromoethyl)benzoate

To a stirred mixture of methyl 4-bromo-2-ethylbenzoate (4.3 g, 17.7 mmol, 1.0 eq) in CCl₄ (40 mL) were added BPO (857 mg, 3.54 mmol, 0.2 eq) and NBS (3.46 g, 19.5 mmol, 1.1 eq). The resulting mixture was stirred under N₂ at 80° C. for 3 h. After evaporation, the residue was diluted with EA (200 mL), washed with saturated aqueous Na₂SO₃ solution (100 mL×2), saturated aqueous NaHCO₃ solution (100 mL×2) and brine (100 mL), dried over anhydrous sodium sulfate, filtered and concentrated under reduced pressure. The residue was purified by flash column chromatography on silica gel (PE/EtOAc=5/1 v/v) to give methyl 4-bromo-2-(1-bromoethyl)benzoate (4.2 g, yield 74%) as a yellow oil. LC-MS (ESI): mass calcd. for C₁₀H₁₀Br202, 319.90; m/z found, no mass [M+H]⁺.

### Step C: 3-(5-bromo-3-methyl-1-oxoisoindolin-2-yl) piperidine-2,6-dione

To a solution of methyl 4-bromo-2-(1-bromoethyl)benzoate (2.0 g, 6.21 mmol, 10 eq) and 3-anminopiperidine-2.6-dione hydrochloride (1.53 g, 9.32 mmol, 1.5 eq) in MeCN (20 mL) was added DIPEA (2.41 g, 3.3 mL, 18.6 mmol, 3.0 eq). The mixture was stirred under N₂ at 85° C. for 16 h. After evaporation, the residue was purified by flash column chromatography on silica gel column (100% ethyl acetate) to give 3-(5-bromo-3-methyl-1-oxoisoindolin-2-yl) piperidine-2,6-dione (1.0 g, yield 48%) as a grey solid. LC-MS (ESI): mass calcd. for C₁₄H₁₃BrN₂O₃, 336.01; m/z found, 337.0 [M+H]⁺. ¹H NMR (400 MHZ, DMSO-d₆) δ 11.00-10.66 (m, 1H), 7.98-7.83 (m, 1H), 7.74-7.56 (m, 2H), 4.96-4.50 (m, 2H), 2.89-2.69 (m, 1H), 2.68-2.52 (m, 2H), 2.03-1.98 (m, 1H), 1.58-1.39 (m, 3H).

### Step D: 3-(3-methyl-1-oxo-5-(4,4,5,5-tetramethyl-1,3,2-dioxaborolan-2-yl) isoindolin-2-yl) piperidine-2,6-dione

To a solution of 3-(5-bromo-3-methyl-1-oxoisoindolin-2-yl) piperidine-2,6-dione (1.1 g, 3.26 mmol, 1.0 eq), anhydrous potassium aetate (961 mg, 9.79 mmol, 3.0 eq), and 4,4,4′,4′,5,5,5′,5′-octamethyl-2,2′-bi (1,3,2-dioxaborolane) (1.24 g, 4.89 mmol, 1.5 eq) in dry dioxane (10 mL) was added Pd(dppf)Cl₂ (239 mg, 326 μmol, 0.1 eq). The reaction mixture was stirred under N₂ at 100° C. for 16 h. After evaporation, The residue was washed with water (50 mL) and EA (10 mL), dried to give 3-(3-methyl-1-oxo-5-(4,4,5,5-tetramethyl-1,3,2-dioxaborolan-2-yl) isoindolin-2-yl) piperidine-2,6-dione (900 mg, yield 72%) as a gray solid. LC-MS (ESI): mass calcd. for C₂₀H₂₅BN₂O₅, 384.19; m/z found, 385.0 [M+H]⁺. ¹H NMR (400 MHz, DMSO-d₆) δ 10.93 (d, J=12.4 Hz, 1H), 7.93-7.87 (m, 1H), 7.83-7.75 (m, 1H), 7.71-7.63 (m, 1H), 4.86-4.59 (m, 2H), 2.82-2.75 (m, 1H), 2.62-2.56 (m, 2H), 2.03-1.99 (m, 1H), 1.46-1.42 (m, 3H), 1.32 (s, 12H).

### Intermediate 29:6-chloro-1-(oxetan-3-yl)-1H-pyrrolo[2,3-b]pyridine-4-carbaldehyde

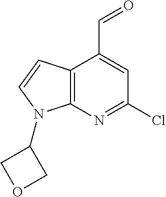 [SMILES: O=Cc1cc(Cl)nc2c1ccn2C1COC1]

### Step A: methyl 6-chloro-1-(oxetan-3-yl)-1H-pyrrolo[2,3-b]pyridine-4-carboxylate

To a solution of methyl 6-chloro-1H-pyrrolo[2,3-b]pyridine-4-carboxylate (1.10 g, 5.22 mmol, 1.0 eq) in DMF (30 mL) were added Cs₂CO₃ (5.10 g, 15.7 mmol, 3.0 eq) and 3-iodooxetane (2.40 g, 13.1 mmol, 2.5 eq) and the reaction mixture was stirred under N₂ at 80° C. for 16 h. After cooled to room temperature, the reaction mixture was filtered and the cake was washed with EA (100 mL×2). The combined filtrates were washed with brine (100 mL×4), dried over anhydrous sodium sulfate, filtered and concentrated under reduced pressure. The crude product was purified by flash column chromatography on silica gel (ethyl acetate in petroleum ether, from 0% to 20% v/v) to afford methyl 6-chloro-1-(oxetan-3-yl)-1H-pyrrolo[2,3-b]pyridine-4-carboxylate (650 mg, yield 47%) as a yellow solid. LC-MS (ESI): mass calcd. for C₁₂H₁₁ClN₂O₃, 266.05; m/z found, 267.0 [M+H]⁺. ¹H NMR (DMSO-d₆) δ 8.26 (d, J=3.6 Hz, 1H), 7.62 (s, 1H), 7.01 (d, J=3.6 Hz, 1H), 6.01-5.88 (m, 1H), 5.05-4.94 (m, 4H), 3.97 (s, 3H).

### Step B: (6-chloro-1-(oxetan-3-yl)-1H-pyrrolo[2,3-b]pyridin-4-yl) methanol

To a solution of methyl 6-chloro-1-(oxetan-3-yl)-1H-pyrrolo[2,3-b]pyridine-4-carboxylate (2.00 g, 7.50 mmol, 1.0 eq) in THF (40.0 mL) was added LiAlH4 (427 mg, 11.2 mmol, 1.5 eq) in portions at 0° C. and the reaction was stirred at 0° C. for 1 h. The reaction was diluted with THF (50 mL), quenched with sodium sulfate decahydrate and stirred for 1 h. The mixture was filtered and the filtrate was concentrated under reduced pressure to afford (6-chloro-1-(oxetan-3-yl)-1H-pyrrolo[2,3-b]pyridin-4-yl) methanol (1.80 g, yield 98%) as a yellow solid. The crude product was directly used in next step without further purification. LC-MS (ESI): mass calced for: C₁₁H₁₁ClN₂O₂, 238.05; m/z found, 239.0 [M+H]⁺.

### Step C: 6-chloro-1-(oxetan-3-yl)-1H-pyrrolo[2,3-b]pyridine-4-carbaldehyde

To a solution of (6-chloro-1-(oxetan-3-yl)-1H-pyrrolo[2,3-b]pyridin-4-yl) methanol (1.80 g, 7.32 mmol, 1.0 eq) in DMSO (20.0 mL) was added IBX (8.19 g, 50% wt, 14.6 mmol, 2.0 eq) in portions and the reaction mixture was stirred at room temperature for 1 h. The reaction solution was diluted with ethyl acetate (150 mL), washed with saturated aqueous Na₂S₂O₃ solution (100 mL×2), saturated aqueous sodium bicarbonate solution (100 mL) and brine (100 mL), dried over anhydrous sodium sulfate, filtered and concentrated under reduced pressure. The residue was purified by flash column chromatography on silica gel (ethyl acetate in petroleum ether, from 10% to 20%) to give 6-chloro-1-(oxetan-3-yl)-1H-pyrrolo[2,3-b]pyridine-4-carbaldehyde (500 mg, yield 29%) as a yellow solid. LC-MS (ESI): mass calced for: C₁₁H₉ClN₂O₂, 236.04; m/z found, 237.1 [M+H]⁺. ¹H NMR (400 MHZ, DMSO-d₆) δ 10.31 (s, 1H), 8.30 (d, J=3.6 Hz, 1H), 7.78 (s, 1H), 7.14 (d, J=3.6 Hz, 1H), 6.00-5.93 (m, 1H), 5.04 (t, J=7.4 Hz, 2H), 4.97 (t, J=7.4 Hz, 2H).

### Intermediate 30:6-chloro-1-cyclopropyl-1H-pyrrolo[2,3-b]pyridine-4-carbaldehyde

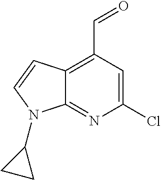 [SMILES: O=Cc1cc(Cl)nc2c1ccn2C1CC1]

### Step A: methyl 6-chloro-1-cyclopropyl-1H-pyrrolo[2,3-b]pyridine-4-carboxylate

To a solution of methyl 6-chloro-1H-pyrrolo[2,3-b]pyridine-4-carboxylate (1.50 g, 7.12 mmol, 1.0 eq) in DCM (20.0 mL) and DMF (5 mL) were added Cupric acetate monohydrate (2.84 g, 14.2 mmol, 2.0 eq), cyclopropylboronic acid (3.06 g, 35.6 mmol, 5.0 eq), and TEA (7.21 g, 9.93 mL, 71.2 mmol, 10.0 eq). The reaction mixture was stirred under N₂ (contained O₂) at 40° C. for 16 h. After cooled to room temperature, the reaction mixture was filtered and he filtrate was diluted with DCM (150 mL). The filtrate was washed with brine (100 mL×4), dried over anhydrous sodium sulfate, filtered and concentrated under reduced pressure. The crude product was purified by flash column chromatography on silica gel (ethyl acetate in petroleum ether, from 0% to 30% v/v) to afford methyl 6-chloro-1-cyclopropyl-1H-pyrrolo[2,3-b]pyridine-4-carboxylate (700 mg, yield 39%) as a white solid. LC-MS (ESI): mass calced for: C₁₂H₁₁ClN₂O₂, 250.05; m/z found, 251.1 [M+H]⁺.

### Step B: (6-chloro-1-(oxetan-3-yl)-1H-pyrrolo[2,3-b]pyridin-4-yl) methanol

To a solution of methyl 6-chloro-1-cyclopropyl-1H-pyrrolo[2,3-b]pyridine-4-carboxylate (700 mg, 2.79 mmol, 1.0 eq) in THF (10.0 mL) was added aluminum (III) lithium hydride (117 mg, 3.07 mmol, 1.1 eq) in portions at 0° C. and the mixture was stirred at 0° C. for 10 min. The reaction mixture was quenched with saturated aqueous NH₄Cl solution (15 mL) at 0° C. and extracted with EA (15 mL×3). The organic layer was washed with H₂O (15 mL×3) and brine (15 mL), dried over anhydrous Na₂SO₄, filtered and concentrated under reduced pressure to give (6-chloro-1-cyclopropyl-1H-pyrrolo[2,3-b]pyridin-4-yl) methanol (600 mg, yield 96%) as a yellow solid. The crude product was directly used in next step without further purification. LC-MS (ESI): mass calced for: C₁H₁ClN₂O, 222.06; m/z found, 223.0 [M+H]⁺.

### Step C: 6-chloro-1-cyclopropyl-1H-pyrrolo[2,3-b]pyridine-4-carbaldehyde

To a solution of (6-chloro-1-cyclopropyl-1H-pyrrolo[2,3-b]pyridin-4-yl) methanol (700 mg, 3.14 mmol, 1.0 eq) in DMSO (20.0 mL) was added IBX (2.64 g, 9.43 mmol, 3.0 eq) in portions at room temperature and the mixture was stirred at room temperature for 1 h. The reaction mixture was quenched with saturated aqueous Na₂S₂O₃ solution (50 mL) and extracted with EA (35 mL×3). The organic layer was washed with saturated aqueous NaHCO₃ solution (40 mL) and brine (40 mL×3), dried over anhydrous Na₂SO₄, filtered and concentrated under reduced pressure. The residue was purified by flash column chromatography on silica gel (DCM/EA=1/3 v/v) to give 6-chloro-1-cyclopropyl-1H-pyrrolo[2,3-b]pyridine-4-carbaldehyde (400 mg, yield 52%) as a yellow solid. LC-MS (ESI): mass calced for: C₁₁H₉ClN₂O 220.04; m/z found, 221.1 [M+H]⁺. ¹H NMR (400 MHZ, CDCl₃) δ 10.26 (s, 1H), 7.51 (s, 1H), 7.39 (d, J=3.6 Hz, 1H), 7.02 (d, J=3.6 Hz, 1H), 3.61-3.55 (m, 1H), 1.19-1.15 (m, 2H), 1.07-1.05 (m, 1H).

### Intermediate 31:6-chloro-4-(pyrrolidin-1-ylmethyl)-1H-pyrrolo[2,3-b]pyridine

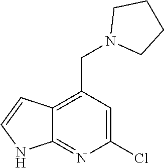 [SMILES: Clc1cc(CN2CCCC2)c2cc[nH]c2n1]

To a solution of 6-chloro-1H-pyrrolo[2,3-b]pyridine-4-carbaldehyde (1.50 g, 8.31 mmol, 1.0 eq) in DCM (30.0 mL) were added pyrrolidine (886 mg, 12.5 mmol, 1.5 eq) and AcOH (748 mg, 713 μL, 12.5 mmol, 1.5 eq) under N₂ at 25° C. and the mixture was stirred at 25° C. for 16 h. Then Sodium triacetoxyborohydride (3.52 g, 16.6 mmol, 2.0 eq) was added to above mixture and the resulting mixture was stirred at 25° C. for 0.5 h. The reaction mixture was quenched with H₂O (20 mL) and extracted with DCM (30 mL×3). The organic layer was washed with saturated aqueous NaHCO₃ solution (30 mL×2) and brine (30 mL), dried over anhydrous Na₂SO₄, filtered and concentrated under reduced pressure. The residue was purified by flash column chromatography on silica gel (ethyl acetate in petroleum ether, from 0% to 20% v/v) to afford 6-chloro-4-(pyrrolidin-1-ylmethyl)-1H-pyrrolo[2,3-b]pyridine (1.64 g, yield 84%)) as a yellow solid. LC-MS (ESI): mass calcd. for C₁₂H₁₄ClN₃, 235.09; m/z found, 236.1 [M+H]⁺. ¹H NMR (400 MHZ, DMSO-d₆) δ 11.82 (s, 1H), 7.45 (s, 1H), 7.07 (s, 1H), 6.59 (s, 1H), 3.86 (s, 2H), 2.50-2.46 (m, 4H), 1.72 (s, 4H).

### Intermediate 32:3-(4-chloro-1-oxo-5-(4,4,5,5-tetramethyl-1,3,2-dioxaborolan-2-yl) isoindolin-2-yl) piperidine-2,6-dione

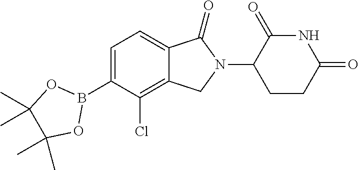 [SMILES: CC1(C)OB(c2ccc3c(c2Cl)CN(C2CCC(=O)NC2=O)C3=O)OC1(C)C]

### Step A: methyl 4-bromo-3-chloro-2-methylbenzoate

To a solution of 4-bromo-3-chloro-2-methylbenzoic acid (5.00 g, 20.0 mmol, 1.0 eq) in DMF (25.0 mL) were added potassium carbonate (5.54 g, 40.1 mmol, 2.0 eq) and iodomethane (4.27 g, 1.87 mL, 30.1 mmol, 1.5 eq). The mixture was stirred at room temperature for 16 h. After filtered, the filtrate was diluted with water (100 mL) and EA (100 mL×4). The organic phase was washed with brine (100 mL×4), dried over anhydrous Na₂SO₄, filtered and concentrated under reduced pressure to give methyl 4-bromo-3-chloro-2-methylbenzoate (5.00 g, yield 95%) as a yellow solid. LC-MS (ESI): mass calcd. for C₉H₈BrClO₂, 261.94; m/z found, no mass [M+H]⁺. ¹H NMR (400 MHZ, DMSO-d₆) δ 7.76 (d, J=8.4 Hz, 1H), 7.63-7.56 (m, 1H), 3.85 (s, 3H), 2.58 (s, 3H).

### Step B: methyl 4-bromo-2-(bromomethyl)-3-chlorobenzoate

To a solution of methyl 4-bromo-3-chloro-2-methylbenzoate (2.50 g, 9.49 mmol, 1.0 eq) and NBS (1.69 g, 9.49 mmol, 1.0 eq) in CCl₄ (50.0 mL) was added BPO (460 mg, 1.9 mmol, 0.2 eq). The reaction mixture was stirred at 90° C. for 16 h. After evaporation, the residue was purified by flash column chromatography on silica gel (ethyl acetate in petroleum ether, from 0% to 5% v/v) to give methyl 4-bromo-2-(bromomethyl)-3-chlorobenzoate (3.00 g, yield 92%) as a white solid. LC-MS (ESI): mass calcd. for C₉H₇Br2ClO₂, 339.85; m/z found, no mass [M+H]⁺

### Step C: 3-(5-bromo-4-chloro-1-oxoisoindolin-2-yl) piperidine-2,6-dione

To a solution of methyl 4-bromo-2-(bromomethyl)-3-chlorobenzoate (4.50 g, 13.1 mmol, 1.0 eq) and 3-aminopiperidine-2,6-dione HCl (3.24 g, 19.7 mmol, 1.5 eq) in MeCN (40.0 mL) was added N-ethyl-N-isopropylpropan-2-amine (5.10 g, 6.87 mL, 39.4 mmol, 3.0 eq). The reaction mixture was heated to 85° C. and stirred for 16 h. After evaporation, the reaction mixture was slurred with EA (40 mL) and water (10 mL). After filtered, the cake was washed with tert-Butyl methyl ether and dried to give 3-(5-bromo-4-chloro-1-oxoisoindolin-2-yl) piperidine-2,6-dione (3.00 g, yield 64%) as a blue solid. LC-MS (ESI): mass calcd. for C₁₃H₁₀BrClN₂O₃, 355.96; m/z found, 357.4 [M+H]⁺. ¹H NMR (400 MHZ, DMSO-d₆) δ 11.01 (s, 1H), 7.95 (d, J=8.0 Hz, 1H), 7.65 (d, J=8.0 Hz, 1H), 5.16-5.11 (m, 1H), 4.54 (d, J=18.0 Hz, 1H), 4.37 (d, J=18.0 Hz, 1H), 2.98-2.85 (m, 1H), 2.60 (d, J=17.4 Hz, 1H), 2.47-2.41 (m, 1H), 2.03-1.98 (m, 1H).

### Step D: 3-(4-chloro-1-oxo-5-(4,4,5,5-tetramethyl-1,3,2-dioxaborolan-2-yl) isoindolin-2-yl) piperidine-2,6-dione

To a solution of 3-(5-bromo-4-chloro-1-oxoisoindolin-2-yl) piperidine-2,6-dione (500 mg, 1.40 mmol, 1.0 eq) and potassium acetate (412 mg, 4.19 mmol, 3.0 eq) in 1,4-Dioxane (10.0 mL) were added 4,4,4′,4′,5,5,5′,5′-octamethyl-2,2′-bi (1,3,2-dioxaborolane) (426 mg, 1.68 mmol, 1.2 eq) and Pd(dppf)Cl₂ (102 mg, 140 μmol, 0.1 eq). The mixture was stirred under N₂ at 95° C. for 16 h. The reaction mixture was cooled to room temperature and concentrated under reduced pressure. The residue was poured into water (20 mL) and extracted with EtOAc (20 mL×3). The organic layer was dried over MgSO₄, filtered and concentrated under reduced pressure. The residue was purified by flash column chromatography on silica gel (DCM/MeOH=10/1 v/v) to obtain 3-(4-chloro-1-oxo-5-(4,4,5,5-tetramethyl-1,3,2-dioxaborolan-2-yl) isoindolin-2-yl) piperidine-2,6-dione (400 mg, yield 78%, 80% Purity) as a brown solid. LC-MS (ESI): mass calcd. for C₁₉H₂₂BClN₂O₅, 404.13; m/z found, 405.1 [M+H]⁺.

### Intermediate 33: tert-butyl(S)-5-amino-5-oxo-4-(1-oxo-5-(4,4,5,5-tetramethyl-1,3,2-dioxaborolan-2-yl) isoindolin-2-yl) pentanoate

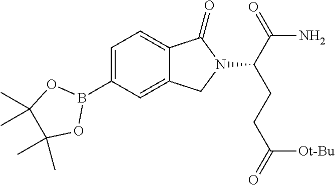 [SMILES: CC(C)(C)OC(=O)CC[C@@H](C(N)=O)N1Cc2cc(B3OC(C)(C)C(C)(C)O3)ccc2C1=O]

### Step A: 4-bromo-2-(hydroxymethyl)benzoic acid

To a solution of 5-bromoisobenzofuran-1 (3H)-one (20.0 g, 93.9 mmol, 1.0 eq) in MeOH (210 mL), THF (210 mL), and Water (70.0 mL) was added NaOH (7.5 g, 188 mmol, 2.0 eq) at 0° C. and the mixture was stirred at 40° C. for 2 h. After evaporated to remove THF/MeOH, the residue was diluted with water (50 mL), and acidified with diluted aqueous HCl solution (1 N) to pH 5-6, and extracted with DCM (300 mL×3). The combined organic layers were washed with brine (300 mL), dried over anhydrous Na₂SO₄, filtered and concentrated under reduced pressure to give 4-bromo-2-(hydroxymethyl)benzoic acid (20.0 g, yield 92%) as a yellow solid. The crude product was directly used in the next step without further purification. LC-MS (ESI): mass calcd. for C₈H₇BrO₃, 229.96; m/z found, 253.0 [M+Na]⁺. ¹H NMR (400 MHZ, DMSO-d₆): δ7.89-7.86 (m, 1H), 7.79 (d, J=8.2 Hz, 1H), 7.55 (dd, J=8.2, 2.0 Hz, 1H), 4.83 (s, 2H).

### Step B: 5-bromo-3-hydroxyisobenzofuran-1 (3H)-one

To a solution of 4-bromo-2-(hydroxymethyl)benzoic acid (6.00 g, 26.0 mmol, 1.0 eq) in DMF (120 mL) was added active Manganese dioxide (38.4 g, 441 mmol, 27.0 eq) and the mixture was stirred at 30° C. for 16 h. After filtration, the filtrate was concentrated under reduced pressure. The crude product was purified by flash column chromatography on silica gel (DCM/MeOH=10/1 v/v) to provide 5-bromo-3-hydroxyisobenzofuran-1 (3H)-one (5.80 g, yield 97%) as a yellow solid. LC-MS (ESI): mass calcd. for C₈H₅BrO₃, 227.94; m/z found, 227.0 [M−H]⁻. ¹H NMR (400 MHZ, DMSO-d₆): δ7.93 (d, J=1.6 Hz, 1H), 7.86 (dd, J=8.0, 1.6 Hz, 1H), 7.77 (d, J=8.0 Hz, 1H), 7.13 (s, 1H).

### Step C: tert-butyl(S)-5-amino-4-(5-bromo-1-oxoisoindolin-2-yl)-5-oxopentanoate

To a solution of 5-bromo-3-hydroxyisobenzofuran-1 (3H)-one (7.60 g, 33.2 mmol, 1.0 eq), tert-butyl(S)-4,5-diamino-5-oxopentanoate hydrochloride (10.3 g, 43.1 mmol, 1.3 eq), and Acetic acid (19.9 g, 19.1 mL, 332 mmol, 10.0 eq) in DMF (150 mL) was added Sodium triacetoxyborohydride (21.1 g, 99.6 mmol, 3.0 eq) at room temperature. The reaction mixture was stirred at 35° C. for 16 h. The mixture was quenched with saturated aqueous NH₄Cl solution (300 mL) and extracted with EtOAc (300 mL×3). The combined organic layers were washed with brine (300 mL×4), dried over anhydrous Na₂SO₄, filtered and concentrated under reduced pressure. The crude product was purified by flash column chromatography on silica gel (PE/EA=1/1 v/v) to provide tert-butyl(S)-5-amino-4-(5-bromo-1-oxoisoindolin-2-yl)-5-oxopentanoate (7.80 g, yield 59%) as a white solid. LC-MS (ESI): mass calcd. for C₁₇H₂₁BrN₂O₄, 396.07; m/z found, 419.1 [M+Na]⁺. ¹H NMR (400 MHZ, DMSO-d₆): δ7.88 (d, J=1.6 Hz, 1H), 7.69 (dd, J=8.0, 1.6 Hz, 1H), 7.64 (d, J=8.0 Hz, 1H), 7.59 (s, 1H), 7.21 (s, 1H), 4.75-4.70 (m, 1H), 4.61 (d, J=18.0 Hz, 1H), 4.47 (d, J=18.0 Hz, 1H), 2.21-2.11 (m, 3H), 2.04-1.93 (m, 1H), 1.33 (s, 9H).

### Step D: tert-butyl(S)-5-amino-5-oxo-4-(1-oxo-5-(4,4,5,5-tetramethyl-1,3,2-dioxaborolan-2-yl) isoindolin-2-yl) pentanoate

To a suspension of tert-butyl(S)-5-amino-4-(5-bromo-1-oxoisoindolin-2-yl)-5-oxopentanoate (2.00 g, 5.03 mmol, 1.0 eq), Bis(pinacolato)diborane (1.53 g, 6.04 mmol, 1.2 eq), and anhydrous Potassium acetate (1.48 g, 15.1 mmol, 3.0 eq) in 1,4-Dioxane (50.0 mL) was added 1,1′-Bis(diphenylphosphino) ferrocene-palladium (II) dichloride (368 mg, 503 μmol, 0.1 eq). The reaction mixture was stirred under nitrogen at 95° C. for 16 h. After evaporation, the crude product was washed with water (20 mL), a mixed solvent (40 mL) (PE/EA=3/1 v/v), and dried to give tert-butyl(S)-5-amino-5-oxo-4-(1-oxo-5-(4,4,5,5-tetramethyl-1,3,2-dioxaborolan-2-yl) isoindolin-2-yl) pentanoate (1.70 g, yield 76%) as a gray solid. LC-MS (ESI): mass calcd. for C₂₃H₃₃BN206, 444.24; m/z found, 445.3 [M+H]⁺. ¹H NMR (400 MHZ, DMSO-d₆): δ7.90 (s, 1H), 7.78 (d, J=7.6 Hz, 1H), 7.70 (d, J=7.6 Hz, 1H), 7.58 (s, 1H), 7.20 (s, 1H), 4.77-4.73 (m, 1H), 4.61 (d, J=17.8 Hz, 1H), 4.47 (d, J=17.8 Hz, 1H), 2.17-2.14 (m, 3H), 2.04-1.89 (m, 1H), 1.33 (t, J=3.8 Hz, 21H).

### Intermediate 34:3-(6-chloro-1-oxo-5-(4,4,5,5-tetramethyl-1,3,2-dioxaborolan-2-yl) isoindolin-2-yl) piperidine-2,6-dione

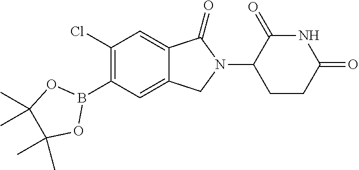 [SMILES: CC1(C)OB(c2cc3c(cc2Cl)C(=O)N(C2CCC(=O)NC2=O)C3)OC1(C)C]

### Step A: methyl 4-bromo-5-methoxy-2-methylbenzoate

To a solution of 4-bromo-5-fluoro-2-methylbenzoic acid (1.50 g, 6.0 mmol, 1.0 eq) in DMF (20.0 mL) was added potassium carbonate (1.66 g, 12.0 mmol, 2.0 eq) and the reaction mixture was stirred at 25° C. for 2 h. Then iodomethane (0.56 mL, 9.0 mmol, 1.5 eq) was added to above mixture and the resulting mixture was stirred at 25° C. for 10 h. The reaction mixture was diluted with EA (100 mL), washed with brine (30 mL×4), dried over anhydrous Na₂SO₄, and filtered. The filtrate was concentrated under reduced pressure to give methyl 4-bromo-5-chloro-2-methylbenzoate (1.40 g, yield 88%) as a white solid. LC-MS (ESI): mass calcd. for C₉H₈BrClO₂, 261.94; m/z found, No signal. ¹H NMR (400 MHZ, DMSO-d₆) δ 7.95 (s, 1H), 7.83 (s, 1H), 3.84 (s, 3H), 2.49 (s, 3H).

### Step B: methyl 4-bromo-2-(bromomethyl)-5-chlorobenzoate

To a solution of methyl 4-bromo-5-chloro-2-methylbenzoate (1.4 g, 5.31 mmol, 1.0 eq) in CCl₄ (15.0 mL) were added NBS (1.04 g, 5.84 mmol, 1.1 eq) and benzoyl peroxide (257 mg, 1.06 mmol, 0.2 eq). The reaction was stirred under N₂ at 80° C. for 2 h. After cooled to room temperature, the reaction mixture was quenched with water (20 mL) and extracted with EA (30 mL×3). The organic phase was washed with brine (30 mL), dried over anhydrous Na₂SO₄, filtered and concentrated under reduced pressure. The residue was purified by flash column chromatography on silica gel (PE/EA=10/1) to give methyl 4-bromo-2-(bromomethyl)-5-chlorobenzoate (1.4 g, yield 76%). as a white solid. LC-MS (ESI): mass calcd. for C₉H₇Br₂ClO₂, 339.85; m/z found, No signal. ¹H NMR (400 MHZ, DMSO-d₆) δ 8.10 (s, 1H), 8.02 (s, 1H), 4.97 (s, 2H), 3.88 (s, 3H).

### Step C: 3-(5-bromo-6-chloro-1-oxoisoindolin-2-yl) piperidine-2,6-dione

To a stirred solution of methyl 4-bromo-2-(bromomethyl)-5-chlorobenzoate (700 mg, 2.04 mmol, 1.0 eq) in MeCN (20.0 mL) were added 3-aminopiperidine-2,6-dione hydrochloride (505 mg, 3.07 mmol, 1.5 eq) and DIPEA (0.79 g, 1.07 mL, 6.13 mmol, 3.0 eq) at room temperature and the resulting mixture was stirred at 80° C. overnight. After cooled to room temperature, the mixture was quenched with water (30 mL) and extracted with EA (30 mL×3). The combined organic layers were washed with brine (30 mL), dried over anhydrous sodium sulfate, filtered and concentrated under reduced pressure. The residue was purified by flash column chromatography on silica gel (ethyl acetate in petroleum ether, from 0 to 100%) to afford 3-(5-bromo-6-chloro-1-oxoisoindolin-2-yl) piperidine-2,6-dione (370 mg, yield 50%) as a white solid. LC-MS (ESI): mass calcd. for C₁₃H₁₀BrClN₂O₃, 355.96; m/z found, 357.2 [M+H]⁺. ¹H NMR (400 MHZ, DMSO-d₆) δ 11.02 (s, 1H), 8.11 (s, 1H), 7.93 (s, 1H), 5.14-5.09 (m, 1H), 4.47 (d, J=17.8 Hz, 1H), 4.35 (d, J=17.8 Hz, 1H), 2.95-2.86 (m, 1H), 2.65-2.56 (m, 1H), 2.41-2.37 (m, 1H), 2.04-1.99 (m, 1H).

### Step D: 3-(6-chloro-1-oxo-5-(4,4,5,5-tetramethyl-1,3,2-dioxaborolan-2-yl) isoindolin-2-yl) piperidine-2,6-dione

To a stirred solution of 3-(5-bromo-6-chloro-1-oxoisoindolin-2-yl) piperidine-2,6-dione (100 mg, 0.28 mmol, 1.0 eq) in 1,4-Dioxane (5.00 mL) were added Potassium acetate (345 mg, 3.52 mmol, 3.0 eq), Bis(pinacolato)diborane (447 mg, 1.76 mmol, 1.5 eq), and Pd (dppf) Cl₂ (85 mg, 117 μmol, 0.1 eq) at room temperature. The mixture was stirred under N₂ at 90° C. for 3 h. After cooled to room temperature, The mixture was filtered and the filtrate was concentrated under reduced pressure. The residue was purified by flash column chromatography on silica gel (ethyl acetate in petroleum ether, from 0 to 100%) to afford 3-(6-fluoro-1-oxo-5-(4,4,5,5-tetramethyl-1,3,2-dioxaborolan-2-yl) isoindolin-2-yl) piperidine-2,6-dione (150 mg, yield 33%) as a brown solid. LC-MS (ESI): mass calcd. for C₁₉H₂₂BrFN₂O₅, 404.13; m/z found, 405.0 [M+H]⁺.

### Intermediate 35:6-chloro-1-(1,1-dioxidothietan-3-yl)-1H-pyrrolo[2,3-b]pyridine-4-carbaldehyde

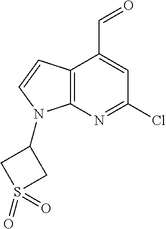 [SMILES: O=Cc1cc(Cl)nc2c1ccn2C1CS(=O)(=O)C1]

### Step A: Methyl 6-chloro-1-(1,1-dioxidothietan-3-yl)-1H-pyrrolo[2,3-b]pyridine-4-carboxylate

To a solution of methyl 6-chloro-1H-pyrrolo[2,3-b]pyridine-4-carboxylate (100 mg, 1 eq, 475 μmol) in DMF (3.00 mL) was added with NaH (14 mg, 570 μmol) at 0° C. 3-bromothietane 1,1-dioxide (105 mg, 570 μmol) was added into the reaction solution after 30 minutes. The mixture was stirred at room temp for 2 hours. The reaction mixture is quenched with ammonium chloride solution, filtered, and the filter cake is washed with water, ethyl acetate, and dried to give methyl 6-chloro-1-(1,1-dioxidothietan-3-yl)-1H-pyrrolo[2,3-b]pyridine-4-carboxylate (100 mg, 318 μmol, 66.9%) as a white solid. LC-MS (ESI): mass calcd. for C₁₂H₁₁ClN₂O₄S, 314.0; m/z found, 315.1 [M+H]⁺. ¹H NMR (400 MHZ, DMSO) δ 8.10 (d, J=3.7 Hz, 1H), 7.66 (s, 1H), 7.01 (d, J=3.7 Hz, 1H), 5.71 (tt, J=9.4, 4.9 Hz, 1H), 4.98-4.88 (m, 2H), 4.78-4.70 (m, 2H), 3.97 (s, 3H).

### Step B: 3-(6-Chloro-4-(hydroxymethyl)-1H-pyrrolo[2,3-b]pyridin-1-yl) thietane 1,1-dioxide

To a solution of methyl 6-chloro-1-(1,1-dioxidothietan-3-yl)-1H-pyrrolo[2,3-b]pyridine-4-carboxylate (100 mg, 318 μmol) in THF (8.00 mL) and MeOH (8.00 mL) was added LiBH₄ (635 μL, 2.00 N in THF, 1.27 mmol) at 0° C. The mixture was stirred at 50° C. for 2 hours. The reaction mixture is quenched with ammonium chloride solution (20 mL) and extracted with ethyl acetate (15 mL×3). The organic layer was washed with brine (30 mL), dried over anhydrous Na₂SO₄, filtered and concentrated under reduced pressure to give 3-(6-chloro-4-(hydroxymethyl)-1H-pyrrolo[2,3-b]pyridin-1-yl) thietane 1,1-dioxide (100 mg, 100%) as a yellow solid. LC-MS (ESI): mass calcd. for C₁₁H₁₁ClN₂O₃S, 286.7; m/z found, 287.1 [M+H]⁺.

### Step C: 6-chloro-1-(1,1-dioxidothietan-3-yl)-1H-pyrrolo[2,3-b]pyridine-4-carbaldehyde

To a solution of 3-(6-chloro-4-(hydroxymethyl)-1H-pyrrolo[2,3-b]pyridin-1-yl) thietane 1,1-dioxide (100 mg, 349 μmol) in DMSO (3.00 mL) was added IBX (244 mg, 872 μmol) at 0° C. The mixture was stirred at room temp for 1 hour. The reaction mixture is quenched with Na₂S₂O₃ (10 mL) and extracted with ethyl acetate (5 mL×3). The organic layer was washed with brine (10 mL), dried over anhydrous Na₂SO₄, filtered and concentrated under reduced pressure to give 6-chloro-1-(1,1-dioxidothietan-3-yl)-1H-pyrrolo[2,3-b]pyridine-4-carbaldehyde (110 mg, 100%) as a yellow solid. . . . LC-MS (ESI): mass calcd. for C₁₁H₉ClN₂O₃S, 284.7; m/z found, 285.1 [M+H]⁺.

### Intermediate 36:6-chloro-4-(pyrrolidin-1-ylmethyl)-1H-pyrazolo[3,4-b]pyridine

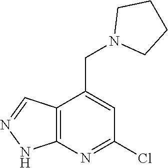 [SMILES: Clc1cc(CN2CCCC2)c2cn[nH]c2n1]

### Step A: ethyl 6-hydroxy-1H-pyrazolo[3,4-b]pyridine-4-carboxylate

To a solution of 1H-pyrazol-3-amine (5.00 g, 60.2 mmol, 1.0 eq) in H₂O (120 mL) were added sodium (Z)-1,4-diethoxy-1,4-dioxobut-2-en-2-olate (12.6 g, 60.2 mmol, 1.0 eq) and AcOH (40.0 mL) and the mixture was stirring at 85° C. for 16 h. After evaporation, the residue was poured into water (60 mL) and extracted with EA (60 mL×3). The organic layers were washed with brine (50 mL), dried over anhydrous Na₂SO₄, filtered and concentrated under reduced pressure. The crude product was purified by flash column chromatography on silica gel (ethyl acetate in petroleum ether, from 0% to 50% v/v) to obtain ethyl 6-hydroxy-1H-pyrazolo[3,4-b]pyridine-4-carboxylate (3.50 g, yield 25%) as a yellow solid. LC-MS (ESI): mass calcd. for C₉H₉N₃O₃, 207.0; m/z found, 208.2 [M+H]⁺. ¹H NMR (400 MHZ, DMSO-d₆) δ 13.33 (s, 1H), 12.12 (s, 1H), 8.16 (s, 1H), 6.67 (s, 1H), 4.36 (q, J=7.2 Hz, 2H), 1.35 (t, J=7.2 Hz, 3H).

### Step B: ethyl 6-chloro-1H-pyrazolo[3,4-b]pyridine-4-carboxylate

To a solution of ethyl 6-hydroxy-1H-pyrazolo[3,4-b]pyridine-4-carboxylate (2.00 g, 9.65 mmol, 1.0 eq) in toluene (50.0 mL) was added DBU (1.76 g, 11.6 mmol, 1.2 eq) and the mixture was stirring at room temperature for 10 min. Then phosphoryl trichloride (1.63 g, 10.6 mmol, 1.1 eq) was added to above mixture and the resulting mixture was stirred at 110° C. for 4 h. After cooled to room temperature, the residue was slowly quenched with saturated aqueous NaHCO₃ solution (20 mL) and extracted with EtOAc (40 mL×4). The organic layer was washed with brine (40 mL), dried over anhydrous MgSO₄, filtered and concentrated under reduced pressure. The crude product was purified by flash column chromatography on silica gel (ethyl acetate in petroleum ether, from 0% to 50% v/v) to obtain ethyl 6-chloro-1H-pyrazolo[3,4-b]pyridine-4-carboxylate (1.20 g, yield 50%) as a white solid. LC-MS (ESI): mass calcd. for C₉H₈ClN₃O₂, 225.0; m/z found, 226.3 [M+H]⁺.

### Step C: (6-chloro-1H-pyrazolo[3,4-b]pyridin-4-yl) methanol

To a solution of ethyl 6-chloro-1H-pyrazolo[3,4-b]pyridine-4-carboxylate (1.00 g, 4.43 mmol, 1.0 eq) in anhydrous THF (20.0 mL) was added dropwise DIBAL-H (1 N in THF) (6.65 mL, 6.65 mmol, 1.5 eq) under N₂ at −78° C. over 10 min. Then the mixture was warmed to 0° C. and stirred at this temperature for 1 h. The reaction mixture was warmed to room temperature, slowly quenched with H₂O (15 mL), and extracted with EtOAc (20 mL×3). The organic layer was dried over anhydrous MgSO₄, filtered and concentrated under reduced pressure. The residue was purified by flash column chromatography on silica gel (ethyl acetate in petroleum ether, from 0% to 33% v/v) to obtain (6-chloro-1H-pyrazolo[3,4-b]pyridin-4-yl) methanol (500 mg, yield 55%) as a yellow solid. LC-MS (ESI): mass calcd. for C₇H₆ClN₃O, 183.0; m/z found, 184.2 [M+H]⁺.

### Step D: 6-chloro-1H-pyrazolo[3,4-b]pyridine-4-carbaldehyde

To a solution of (6-chloro-1H-pyrazolo[3,4-b]pyridin-4-yl) methanol (300 mg, 1.63 mmol, 1.0 eq) in DMSO (10.0 mL) was added IBX (1.14 g, 4.08 mmol, 2.5 eq) and the mixture was stirred at room temperature for 1 h. The mixture was quenched with aqueous Na₂CO₃ solution (5 mL), diluted with water (6 mL), and extracted with EA (10 mL×3). The organic layer was washed with brine (10 mL×4), dried over anhydrous sodium sulfate, filtered and concentrated under reduced pressure. The residue was purified by flash column chromatography on silica gel (ethyl acetate in petroleum ether, from 0% to 33%) to obtain 6-chloro-1H-pyrazolo[3,4-b]pyridine-4-carbaldehyde (150 mg, yield 46%) as a yellow solid. LC-MS (ESI): mass calcd. for C₇H₄ClN₃O, 181.0; m/z found, 182.2 [M+H]⁺. ¹H NMR (400 MHZ, DMSO-d₆) δ 14.28 (s, 1H), 10.28 (s, 1H), 8.52 (s, 1H), 7.90 (s, 1H).

### Step E: 6-chloro-4-(pyrrolidin-1-ylmethyl)-1H-pyrazolo[3,4-b]pyridine

To a solution of 6-chloro-1H-pyrazolo[3,4-b]pyridine-4-carbaldehyde (230 mg, 1.27 mmol, 1.0 eq) and pyrrolidine (135 mg, 1.90 mmol, 1.5 eq) in DCM (2.0 mL) was added AcOH (0.1 mL) and the mixture was stirred at room temperature for 1 h. Then sodium triacetoxyborohydride (537 mg, 2.53 mmol, 2.0 eq) was added to above mixture and the mixture was stirred at room temperature overnight. The mixture was poured into water (6 mL) and extracted with DCM (10 mL×3). The organic layer was dried over MgSO₄, filtered and concentrated under reduced pressure. The residue was purified by Prep-TLC(DCM/MeOH=10/1) to give 6-chloro-4-(pyrrolidin-1-ylmethyl)-1H-pyrazolo[3,4-b]pyridine (120 mg, yield 36%) as a yellow oil. LC-MS (ESI): mass calcd. for C₁₁H₁₃ClN4, 236.0; m/z found, 237.1 [M+H]⁺. ¹H NMR (400 MHZ, DMSO-d₆) δ 13.78 (s, 1H), 8.29 (s, 1H), 7.18 (s, 1H), 3.96 (s, 2H), 2.50 (s, 4H), 1.74 (s, 4H).

### Intermediate 37: 6-chloro-1-methyl-4-(1-(pyrrolidin-1-yl)ethyl)-1H-pyrrolo[2,3-b]pyridine

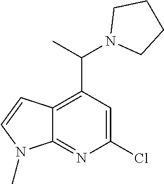 [SMILES: CC(c1cc(Cl)nc2c1ccn2C)N1CCCC1]

### Step A: 1-(6-chloro-1-methyl-1H-pyrrolo[2,3-b]pyridin-4-yl) ethan-1-one

To a solution of 4-bromo-6-chloro-1-methyl-1H-pyrrolo[2,3-b]pyridine (500 mg, 2.04 mmol, 1.0 eq) in 1,4-Dioxane (10.0 mL) were added Bis(dibenzylideneacetone) palladium (143 mg, 204 μmol, 0.1 eq), tributyl (1-ethoxyvinyl) stannane (1.10 g, 3.05 mmol, 1.5 eq), and Potassium phosphate tribasic (1.30 g, 6.11 mmol, 3.0 eq). The mixture was stirred under N₂ at 90° C. for 3 h. The reaction mixture was cooled to room temperature, quenched with water (20 mL), and extracted with EtOAc (50 mL×3). The combined organic phase was washed with brine (50 mL×2), dried over anhydrous Na₂SO₄, filtered and concentrated under reduced pressure to give crude 6-chloro-4-(1-ethoxyvinyl)-1-methyl-1H-pyrrolo[2,3-b]pyridine (400 mg). The crude 6-chloro-4-(1-ethoxyvinyl)-1-methyl-1H-pyrrolo[2,3-b]pyridine (400 mg, 1.69 mmol, 1.0 eq) was dissolved in diluted aqueous HCl solution (2 N) (30 mL) and the reaction mixture was stirred at room temperature for 3 h. The resulting mixture was diluted with EtOAc (100 mL), washed with brine (50 mL×2), dried over Na₂SO₄, filtered and concentrated under reduced pressure. The residue was purified by flash column chromatography on silica gel (ethyl acetate in petroleum ether, from 0% to 40% v/v) to give 1-(6-chloro-1-methyl-1H-pyrrolo[2,3-b]pyridin-4-yl) ethan-1-one (230 mg, yield 63.5%) as a light yellow solid. LC-MS (ESI): mass calcd. for C₁₀H₉ClN₂O, 208.04; m/z found, 209.2 [M+H]⁺. ¹H NMR (400 MHZ, DMSO-d₆) δ 7.94 (d, J=3.4 Hz, 1H), 7.75 (s, 1H), 6.65 (d, J=3.4 Hz, 1H), 3.94 (s, 3H), 2.71 (s, 3H).

### Step B: 6-chloro-1-methyl-4-(1-(pyrrolidin-1-yl)ethyl)-1H-pyrrolo[2,3-b]pyridine

To a solution of 1-(6-chloro-1-methyl-1H-pyrrolo[2,3-b]pyridin-4-yl) ethan-1-one (200 mg, 0.96 mmol, 1.0 eq) in DCM (10.0 mL) were added pyrrolidine (136 mg, 1.92 mmol, 2.0 eq) and AcOH (115 mg, 1.92 mmol, 2.0 eq). The mixture was stirred at room temperature for 30 min. Sodium triacetoxyborohydride (406 mg, 1.92 mmol, 2.0 eq) was added to above mixture and the resulting mixture was stirred at room temperature for 3 h. The mixture was quenched with saturated aqueous NaHCO₃ solution (20 mL) and extracted with DCM (50 mL×3). The combined organic phases were washed with brine (50 mL×2), dried over anhydrous Na₂SO₄, filtered and concentrated under reduced pressure. The residue was purified by flash column chromatography on silica gel (MeOH in DCM, from 0% to 10% v/v) to give 6-chloro-1-methyl-4-(1-(pyrrolidin-1-yl)ethyl)-1H-pyrrolo[2,3-b]pyridine (160 mg, yield 63%) as a light yellow oil. LC-MS (ESI): mass calcd. for C₁₄H₁₈ClN₃, 263.12; m/z found, 264.1 [M+H]⁺.

### Intermediate 38:4-(bromomethyl)-6-chloro-1-methyl-1H-pyrrolo[2,3-b]pyridine

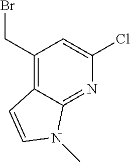

To a solution of (6-chloro-1-methyl-1H-pyrrolo[2,3-b]pyridin-4-yl) methanol (420.0 mg, 2.14 mmol, 1.0 eq) in DCM (10.00 mL) were added PPh3 (560 mg, 2.14 mol, 1.0 eq), imidazole (305 mg, 4.49 mmol, 2.1 eq), and CBr4 (779 mg, 2.35 mmol, 1.1 eq). The mixture was stirred at 10° C. for 2 h. The mixture was diluted with DCM (20 mL), washed with saturated aqueous NaHCO₃ solution (20 mL) and brine (20 mL), dried over anhydrous Na₂SO₄, filtered and concentrated under reduced pressure. The residue was purified by flash column chromatography on silica gel (ethyl acetate in petroleum ether, 5% v/v) to give 4-(bromomethyl)-6-chloro-1-methyl-1H-pyrrolo[2,3-b]pyridine (148 mg, yield 27%) as a yellow solid. LC-MS (ESI): mass calcd. for C₉H₈BrClN₂, 257.96; m/z found, 259.5 [M+H]⁺.

### Intermediate 39: (6-chloro-1-methyl-1H-pyrrolo[2,3-b]pyridin-4-yl) (pyrrolidin-1-yl) methanone

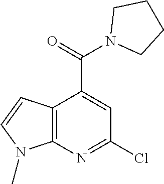 [SMILES: Cn1ccc2c(C(=O)N3CCCC3)cc(Cl)nc21]

### Step A: 6-chloro-1-methyl-1H-pyrrolo[2,3-b]pyridine-4-carboxylic acid

To a solution of methyl 6-chloro-1-methyl-1H-pyrrolo[2,3-b]pyridine-4-carboxylate (260 mg, 1.16 mmol, 1.0 eq) in THE/MeOH/water (2 mL/2 mL/2 mL) was added LiOH (55.4 mg, 2.31 mmol, 2.0 eq) in portions. The reaction mixture was stirred at room temperature for 1 h. After evaporation, the residue was diluted with water (25 mL) and extracted with ethyl acetate (25 mL×2). The aqueous layer was acidified to pH 2.0 with diluted aqueous HCl (1 N) and the precipitated solid was formed. The mixture was filtered and the cake was washed with water (5 mL), and dried under reduced pressure. The solid was slurred with ethyl acetate, filtered, and dried to get 6-chloro-1-methyl-1H-pyrrolo[2,3-b]pyridine-4-carboxylic acid (220 mg, yield 90%) as a white solid. LC-MS (ESI): mass calced for: C₉H₇ClN₂O₂, 224.04; m/z found, 211.1 [M+H]⁺.

### Step B: (6-chloro-1-methyl-1H-pyrrolo[2,3-b]pyridin-4-yl) (pyrrolidin-1-yl) methanone

To a solution of 6-chloro-1-methyl-1H-pyrrolo[2,3-b]pyridine-4-carboxylic acid (110 mg, 522 μmol, 1.0 eq) and pyrrolidine (74.3 mg, 1.04 mmol, 2.0 eq) in DMF (3.0 mL) were added HATU (298 mg, 783 μmol, 1.5 eq) and DIPEA (135 mg, 182 μL, 1.04 mmol, 2.0 eq) and the mixture was stirred at room temperature for 2 h. The mixture was diluted with water (15 mL) and extracted with ethyl acetate (50 mL×3). The organic layer was washed with brine (20 mL×4), dried over anhydrous sodium sulfate, filtered and concentrated under reduced pressure. The residue was purified by flash column chromatography on silica gel (ethyl acetate in petroleum ether, from 33% to 50% v/v) to give (6-chloro-1-methyl-1H-pyrrolo[2,3-b]pyridin-4-yl) (pyrrolidin-1-yl) methanone (65.0 mg, yield 47%) as a yellow oil. LC-MS (ESI): mass calced for: C₁₃H₁₄ClN30 197.00; m/z found, 264.1 [M+H]⁺.

### Intermediate 40:6-chloro-1-methyl-4-(pyrrolidin-1-yl)-1H-pyrrolo[2,3-b]pyridine

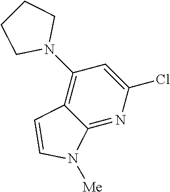 [SMILES: Cn1ccc2c(N3CCCC3)cc(Cl)nc21]

### Step A: 4-bromo-1H-pyrrolo[2,3-b]pyridine 7-oxide

To a solution of 4-bromo-1H-pyrrolo[2,3-b]pyridine (2.0 g, 10.2 mmol, 1.0 eq) in ethyl acete (50.0 mL) was added 3-Chloroperoxybenzoic acid (85%) (3.3 g, 16.2 mmol, 1.6 eq) in portions at 0° C. and the mixture was stirred under N₂ at room temperature for 3 h. The formed precipitate was filtered and the cake was washed with cold water (15 mL×3). The cake was dried to afford 4-bromo-1H-pyrrolo[2,3-b]pyridine 7-oxide (1.3 g, yield 60%) as a white solid. LC-MS (ESI): mass calcd. for C₇H₅BrN₂O, 213.03.; m/z found, 213.4 [M+H]⁺. ¹H NMR (400 MHz, DMSO-d₆) δ 12.87 (s, 1H), 8.08 (d, J=6.5 Hz, 1H), 7.58 (s, 1H), 7.34 (d, J=6.5 Hz, 1H), 6.52 (s, 1H).

### Step B: 4-bromo-6-chloro-1H-pyrrolo[2,3-b]pyridine

To a solution of 4-bromo-1H-pyrrolo[2,3-b]pyridine 7-oxide (1.0 g, 4.69 mmol, 1.0 eq) in DMF (15.0 mL) was added dropwise methanesulfonyl chloride (1.34 g, 11.7 mmol, 2.5 eq) at 50° C. and the mixture was stirred under N₂ at 75° C. for 1 h. After cooled to room temperature, the reaction was quenched with water (25 mL) at 0° C., and neutralised with aqueous sodium hydroxide solution (6 N), and the resulting slurry was stirred at room temperature for 3 h. The formed precipitate was filtered and washed with cold water, dried to afford 4-bromo-6-chloro-1H-pyrrolo[2,3-b]pyridine (0.5 g, yield 46%) as a purple solid. LC-MS (ESI): mass calcd. for C₇H₄BrClN₂, 231.48.; m/z found 232.5 [M+H]⁺. ¹H NMR (400 MHZ, DMSO-d₆) δ 12.27 (s, 1H), 7.64 (p, J=3.2 Hz, 1H), 7.35 (d, J=5.5 Hz, 1H), 6.55 (dd, J=3.5, 1.9 Hz, 1H).

### Step C: 4-bromo-6-chloro-1-methyl-1H-pyrrolo[2,3-b]pyridine

To a solution of 4-bromo-6-chloro-1H-pyrrolo[2,3-b]pyridine (0.5 g, 2.16 mmol, 1.0 eq) in DMF (15 mL) was added NaH (60% suspend in oil) (0.13 g, 3.24 mmol, 1.5 eq) at 0° C. and the mixture was stirred under N₂ at 0° C. for 0.5 h. Then CH₃I (0.3 mL, 4.31 mmol, 2.0 eq) was added to above mixture and the resulting mixture was stirred at room temperature at 0° C. for 2 h. The mixture was quenched with water (10 mL) and extracted with ethyl acetate (15 mL×3). The combined organic layers were washed with brine (10 mL×3), dried over anhydrous Na₂SO₄, and filtered. The filtrate was concentrated under reduced pressure to afford 4-bromo-6-chloro-1-methyl-1H-pyrrolo[2,3-b]pyridine (0.4 g, yield 75%) as a white solid. The crude product was directly used in next step without further purification. LC-MS (ESI): mass calcd. for C₈H₆BrClN₂, 245.50.; m/z found, 246.4 [M+H]⁺. ¹H NMR (400 MHZ, DMSO-d₆) δ 7.70-7.65 (m, 1H), 7.38 (s, 1H), 6.57 (dd, J=3.5, 2.3 Hz, 1H), 3.81 (s, 1H).

### Step D: (6-chloro-2-methyl-2H-pyrazolo[3,4-b]pyridin-4-yl) methanol

To a solution of 4-bromo-6-chloro-1-methyl-1H-pyrrolo[2,3-b]pyridine (50 mg, 0.204 mmol, 1.0 eq), xantphos (23.6 mg, 0.0407 mmol, 0.2 eq) and pyrrolidine (29.0 mg, 0.407 mmol, 2.0 eq) in dioxane (5 mL) were added Pd₂ (dba)₃ (18.7 mg, 0.0204 mmol, 0.1 eq) and Cs₂CO₃ (133 mg, 0.407 mmol, 2.0 eq). The mixture was stirred under N₂ at 80° C. overnight. The reaction mixture was cooled to room temperature and filtered. The filtrate was concentrated and the residue was purified by Prep-TLC(PE/EA=5/1 v/v) to afford 6-chloro-1-methyl-4-(pyrrolidin-1-yl)-1H-pyrrolo[2,3-b]pyridine (10 mg, yield 72%) as as a yellow solid. LC-MS (ESI): mass calcd. for C₁₂H₁₄ClN₃, 235.71; m/z found, 236.2 [M+H]⁺. ¹H NMR (400 MHZ, DMSO-d₆) δ 7.11 (d, J=3.4 Hz, 1H), 6.35 (s, 1H), 6.25 (d, J=3.4 Hz, 1H), 3.66 (s, 3H), 3.23 (s, 4H), 1.97-1.93 (m, 4H).

### Intermediate 41:5-chloro-1-ethyl-1H-pyrrolo[3,2-b]pyridine-7-carbaldehyde

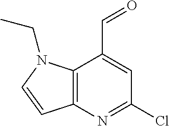

### Step A: methyl 5-chloro-1-ethyl-1H-pyrrolo[3,2-b]pyridine-7-carboxylate

To a solution of methyl 5-chloro-1H-pyrrolo[3,2-b]pyridine-7-carboxylate (500 mg, 2.37 mmol, 1.0 eq) in DMF (10.0 mL) was added NaH (60% suspend in oil) (85 mg, 3.56 mmol, 1.5 eq) at 0° C. and the mixture was stirred at 0° C. for 10 min. Then iodoethane (555 mg, 3.56 mmol, 1.5 eq) was added to above mixture and the resulting mixture was stirred under N₂ at room temperature for 3 h. The mixture was quenched with ice-water (40 mL) and extracted with EtOAc (50 mL×3). The combined organic phases were washed with brine (50 mL×4), dried over anhydrous Na₂SO₄, filtered and concentrated under reduced pressure. The residue was purified by flash column chromatography on silica gel (ethyl acetate in petroleum ether, from 0% to 30% v/v) to give methyl 5-chloro-1-ethyl-1H-pyrrolo[3,2-b]pyridine-7-carboxylate (375 mg, yield 66%) as a white solid. LC-MS (ESI): mass calcd. for C₁₀H₁₁ClN₂O₂, 238.05; m/z found, 239.1 [M+H]⁺.

### Step B: (5-chloro-1-ethyl-1H-pyrrolo[3,2-b]pyridin-7-yl) methanol

To a solution of ethyl 5-chloro-1-ethyl-1H-pyrrolo[3,2-b]pyridine-7-carboxylate (370 mg, 1.46 mmol, 1.0 eq) in THF (10.0 mL) was added LiAlH4 (83.3 mg, 2.20 mmol, 1.5 eq) in portions at 0° C. and the mixture was stirred under N₂ at 0° C. for 1 h. The resulting mixture was diluted with (30 mL), slowly quenched with sodium sulfate decahydrate, and filtered. The filtrate was concentrated under reduced pressure to give (5-chloro-1-ethyl-1H-pyrrolo[3,2-b]pyridin-7-yl) methanol (260 mg, yield 84%) as a light yellow oil. The crude product was directly used in next step without further purification. LC-MS (ESI): mass calcd. for C₁₀H₁₁ClN₂O, 210; m/z found, 211.1 [M+H]⁺.

### Step C: 5-chloro-1-ethyl-1H-pyrrolo[3,2-b]pyridine-7-carbaldehyde

To a solution of (5-chloro-1-ethyl-1H-pyrrolo[3,2-b]pyridin-7-yl) methanol (260 mg, 1.23 mmol, 1.0 eq) in DMSO (5.00 mL) was added IBX (691 mg, 2.47 mmol, 2.0 eq). The mixture was stirred at 30° C. for 3 h, quenched with ice water (15 mL), and extracted with EtOAc (20 mL×3). The organic layer was washed with brine (10 mL×4), dried over anhydrous Na₂SO₄, filtered and concentrated under reduced pressure. The residue was purified by flash column chromatography on silica gel (ethyl acetate in petroleum ether, from 20% to 80% v/v) to afford 5-chloro-1-ethyl-1H-pyrrolo[3,2-b]pyridine-7-carbaldehyde (190 mg, yield 74%) as a white solid. LC-MS (ESI): mass calcd. for C₁₀H₉ClN₂O, 208.04; m/z found, 209.1 [M+H]⁺.

### Intermediate 42:6-chloro-1-cyclobutyl-1H-pyrrolo[2,3-b]pyridine-4-carbaldehyde

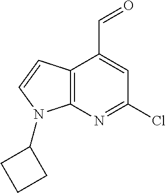 [SMILES: O=Cc1cc(Cl)nc2c1ccn2C1CCC1]

### Step A: methyl 6-chloro-1-(cyanomethyl)-1H-pyrrolo[2,3-b]pyridine-4-carboxylate

To a solution of methyl 6-chloro-1H-pyrrolo[2,3-b]pyridine-4-carboxylate (180 mg, 0.86 mmol, 1.0 eq) in DMF (10.0 mL) were added Cs₂CO₃ (557 mg, 1.71 mmol, 2.0 eq) and bromocyclobutane (173 mg, 1.29 mmol, 1.5 eq). The mixture was stirred under N₂ at 70° C. for 3 h. The resulting mixture was cooled to room temperature, diluted with H₂O (30 mL), and extracted with EA (50 mL×3). The organic layer was washed with brine (20 mL×4), dried over anhydrous Na₂SO₄, filtered and concentrated under reduced pressure. The residue was purified by flash column chromatography on silica gel (ethyl acetate in petroleum ether, from 0% to 30% v/v) to give methyl 6-chloro-1-cyclobutyl-1H-pyrrolo[2,3-b]pyridine-4-carboxylate (190 mg, yield 84%) as an off-white solid. LC-MS (ESI): mass calcd. for C₁₃H₁₃ClN₂O₂, 264.07; m/z found, 265.1 [M+H]⁺.

### Step B: (6-chloro-1-cyclobutyl-1H-pyrrolo[2,3-b]pyridin-4-yl) methanol

To a solution of methyl 6-chloro-1-cyclobutyl-1H-pyrrolo[2,3-b]pyridine-4-carboxylate (190 mg, 0.72 mmol, 1.0 eq) in THF (10.0 mL) was added LiAlH4 (83.3 mg, 2.20 mmol, 1.5 eq) in portions at 0° C. The mixture was stirred under N₂ at 0° C. for 1 h. The resulting mixture was diluted with THF (20 mL), slowly quenched with sodium sulfate decahydrate, and filtered. The filtrate was concentrated under reduced pressure to give (6-chloro-1-cyclobutyl-1H-pyrrolo[2,3-b]pyridin-4-yl) methanol (110 mg, yield 65%) as a light yellow oil. The crude product was directly used in next step without further purification. LC-MS (ESI): mass calcd. for C₁₂H₁₃ClN₂O, 236.07; m/z found, 237.2 [M+H]⁺.

### Step C: 6-chloro-1-cyclobutyl-1H-pyrrolo[2,3-b]pyridine-4-carbaldehyde

To a solution of (6-chloro-1-cyclobutyl-1H-pyrrolo[2,3-b]pyridin-4-yl) methanol (110 mg, 0.47 mmol, 1.0 eq) in DMSO (10.0 mL) was added IBX (210 mg, 0.84 mmol, 1.8 eq) and the resulting mixture was stirred at 30° C. for 3 h. The reaction mixture was quenched with ice water (40 mL) and extracted with EtOAc (50 mL×3). The combined organic phases were washed with brine (50 mL×4), dried over anhydrous Na₂SO₄, filtered and concentrated under reduced pressure. The residue was purified by flash column chromatography on silica gel (ethyl acetate in petroleum ether, from 0% to 70% v/v) to give 6-chloro-1-cyclobutyl-1H-pyrrolo[2,3-b]pyridine-4-carbaldehyde (90 mg, yield 82%) as a white solid. LC-MS (ESI): mass calcd. for C₁₂H₁₁ClN₂O, 234.06; m/z found, 235.1 [M+H]⁺.

### Intermediate 43:6-chloro-1-phenyl-1H-pyrrolo[2,3-b]pyridine-4-carbaldehyde

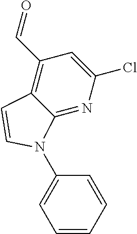 [SMILES: O=Cc1cc(Cl)nc2c1ccn2-c1ccccc1]

### Step A: methyl 6-chloro-1-phenyl-1H-pyrrolo[2,3-b]pyridine-4-carboxylate

To a suspension of methyl 6-chloro-1H-pyrrolo[2,3-b]pyridine-4-carboxylate (200 mg, 950 μmol, 1.0 eq), phenylboronic acid (347 mg, 2.85 mmol, 3.0 eq), and Triethylamine (961 mg, 1.32 mL, 9.50 mmol, 10.0 eq) in DCM (3.00 mL) and DMF (3.00 mL) was added Copper diacetate (345 mg, 1.90 mmol, 2.0 eq). The reaction mixture was stirred under 02 (1 atm) at 40° C. for 16 h. After cooled to room temperature, the mixture was filtered and the filtrate was concentrated in vacuum. The crude product was purified by flash column chromatography on silica gel (PE/EA=10/1 v/v) to provide methyl 6-chloro-1-phenyl-1H-pyrrolo[2,3-b]pyridine-4-carboxylate (200 mg, yield 73%) as a yellow solid. LC-MS (ESI): mass calced for: C₁₅H₁₁ClN₂O₂ 286.05; m/z found, 287.0 [M+H]⁺.

### Step B: (6-chloro-1-phenyl-1H-pyrrolo[2,3-b]pyridin-4-yl) methanol

To a solution of methyl 6-chloro-1-phenyl-1H-pyrrolo[2,3-b]pyridine-4-carboxylate (408 mg, 1.42 mmol, 1.0 eq) in THF (10.0 mL) was added Aluminum lithium hydride (108 mg, 2.85 mmol, 2.0 eq) at 0° C. The reaction mixture was stirred at 0° C. for 20 min. The reaction mixture was quenched with Na₂SO_{4 10}H₂O and stirred for 30 min. After filtration, the filtrate was diluted with water (20 mL) and extracted with ethyl acetate (20 mL×3). The combined organic extracts were washed with water (20 mL) and brine (20 mL), dried over anhydrous sodium sulfate, and filtered. The filtrate was concentrated under reduced pressure to give (6-chloro-1-phenyl-1H-pyrrolo[2,3-b]pyridin-4-yl) methanol (355 mg, yield 96%) as a brown solid, which was used directly to next step. LC-MS (ESI): mass calced for: C₁₄H₁ClN₂O 258.06; m/z found, 259.1 [M+H]⁺

### Step C: 6-chloro-1-phenyl-1H-pyrrolo[2,3-b]pyridine-4-carbaldehyde

To a solution of (6-chloro-1-phenyl-1H-pyrrolo[2,3-b]pyridin-4-yl) methanol (355 mg, 1.37 mmol, 1.0 eq) in DMSO (10.0 mL) was added IBX (Purity 45% W.t) (1.15 g, 4.12 mmol, 3.0 eq) at room temperature. The reaction mixture was stirred at room temperature for 15 min. The reaction mixture was diluted with water (20 mL) and extracted with ethyl acetate (20 mL×3). The combined organic extracts were washed with saturated aqueous Na₂S₂O₃ solution (20 mL×3), saturated aqueous NaHCO₃ solution (20 mL×2) and brine (20 mL), dried over anhydrous sodium sulfate, filtered and concentrated in vacuum. The crude product was purified by flash column chromatography on silica gel (PE/EA=10/1 v/v) to provide 6-chloro-1-phenyl-1H-pyrrolo[2,3-b]pyridine-4-carbaldehyde (112 mg, yield 32%) as a yellow solid. LC-MS (ESI): mass calced for: C₁₄H₉ClN₂O 256.04; m/z found, 257.0 [M+H]⁺.

### Intermediate 44:6-chloro-1-isobutyl-1H-pyrrolo[2,3-b]pyridine-4-carbaldehyde

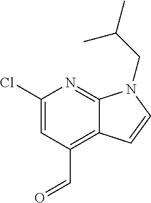 [SMILES: CC(C)Cn1ccc2c(C=O)cc(Cl)nc21]

### Step A: methyl 6-chloro-1-isobutyl-1H-pyrrolo[2,3-b]pyridine-4-carboxylate

To a solution of methyl 6-chloro-1H-pyrrolo[2,3-b]pyridine-4-carboxylate (300 mg, 1.4 mmol, 1.0 eq) and Cs₂CO₃ (928 mg, 2.9 mmol, 2.0 eq) in DMF (5.0 mL) was added 1-iodo-2-methylpropane (315 mg, 1.7 mmol, 1.2 eq) at 20° C. The reaction mixture was stirred at 70° C. for 3 h. After cooled to room temperature, the reaction mixture was quenched with water (20 mL) and extracted with EA (30 mL×3). The organic layer was washed with brine (30 mL×4), dried over anhydrous Na₂SO₄, filtered and concentrated under reduced pressure. The residue was purified by flash column chromatography on silica gel (PE/EA=20/1 v/v) to give methyl 6-chloro-1-isobutyl-1H-pyrrolo[2,3-b]pyridine-4-carboxylate (300 mg, yield 79%) as a yellow oil. LC-MS (ESI): mass calcd. for C₁₃H₁₅ClN₂O₂, 266.1; m/z found, 267.1 [M+H]⁺.

### Step B: (6-chloro-1-isobutyl-1H-pyrrolo[2,3-b]pyridin-4-yl) methanol

To a solution of methyl 6-chloro-1-isobutyl-1H-pyrrolo[2,3-b]pyridine-4-carboxylate (300 mg, 1.1 mmol, 1.0 eq) in THF (6.0 mL) was added LAH (85 mg, 2.3 mmol, 2.0 eq) at 0° C. The reaction mixture was stirred at 0° C. for 2 min. The reaction mixture was quenched with water (20 mL) and extracted with EA (30 mL×3). The organic layer was washed with brine (30 mL), dried over anhydrous Na₂SO₄, filtered and concentrated under reduced pressure. The residue was purified by flash column chromatography on silica gel (PE/EA=2/1 v/v) to give (6-chloro-1-isobutyl-1H-pyrrolo[2,3-b]pyridin-4-yl) methanol (250 mg, yield 93%) as a yellow oil. LC-MS (ESI): mass calcd. for C₁₂H₁₅ClN₂O, 238.1; m/z found, 239.1 [M+H]⁺.

### Step C: 6-chloro-1-isobutyl-1H-pyrrolo[2,3-b]pyridine-4-carbaldehyde

To a solution of (6-chloro-1-isobutyl-1H-pyrrolo[2,3-b]pyridin-4-yl) methanol (200 mg, 838 μmol, 1.0 eq) in DMSO (5.0 mL) was added IBX (Purity 45%˜55%) (704 mg, 2.5 mmol, 3.0 eq) at 25° C. The reaction mixture was stirred at room temperature for 20 min. The reaction mixture was quenched with water (20 mL) and extracted with DCM (20 mL×3). The organic layer was washed with brine (30 mL), dried over Na₂SO₄ and concentrated under reduced pressure. The residue was purified by flash column chromatography on silica gel (PE/EA=20/1 v/v) to give 6-chloro-1-isobutyl-1H-pyrrolo[2,3-b]pyridine-4-carbaldehyde (150 mg, yield 76%) as a yellow oil. LC-MS (ESI): mass calcd. for C₁₂H₁₃ClN₂O, 236.1; m/z found, 237.1 [M+H]⁺.

### Intermediate 45:5-chloro-3-((2-(trimethylsilyl)ethoxy)methyl)-3H-imidazo[4,5-b]pyridine-7-carbaldehyde

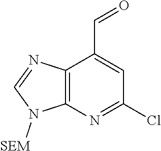 [SMILES: C[Si](C)(C)CCOCn1cnc2c(C=O)cc(Cl)nc21]

### Step A: 7-bromo-5-chloro-3-((2-(trimethylsilyl)ethoxy)methyl)-3H-imidazo[4,5-b]pyridine

To a solution of 7-bromo-5-chloro-3H-imidazo[4,5-b]pyridine (1.20 g, 5.16 mmol, 1.0 eq) in DMF (15.0 mL) was added NaH (60% suspend in oil) (0.15 g, 6.19 mmol, 1.2 eq) at 0° C. After 30 min, 2-(chloromethoxyethyl)trimethyl silane (947 mg, 1.00 mL, 5.68 mmol, 1.1 eq) was added to above mixture and the resulting mixture was stirred at 25° C. for 2 h. The reaction mixture was quenched with saturated aqueous NH₄Cl solution (50 mL) and extracted with EA (20 mL×3). The organic layer was washed with brine (50 mL×4), dried over anhydrous Na₂SO₄, filtered and concentrated under reduced pressure. The crude product was purified by flash column chromatography on silica gel (ethyl acetate in petroleum ether, 12% v/v) to obtain 7-bromo-5-chloro-3-((2-(trimethylsilyl)ethoxy)methyl)-3H-imidazo[4,5-b]pyridine (990 mg, yield 52%) as a colorless oily. LC-MS (ESI): mass calced for: C₁₂H₁₇BrClN₃OSi, 361.00; m/z found, 362.3 [M+H]⁺. ¹H NMR (400 MHZ, DMSO-d₆) δ 8.84 (s, 1H), 7.82 (d, J=43.8 Hz, 1H), 5.71 (s, 2H), 3.68 (t, J=7.6 Hz, 2H), 0.94 (t, J=7.6 Hz, 2H),-0.00 (s, 9H).

### Step B: 5-chloro-3-((2-(trimethylsilyl)ethoxy)methyl)-7-vinyl-3H-imidazo[4,5-b]pyridine

To a solution of 7-bromo-5-chloro-3-((2-(trimethylsilyl)ethoxy)methyl)-3H-imidazo[4,5-b]pyridine (450 mg, 1.24 mmol, 1.0 eq) in 1,4-Dioxane (7.00 mL) and water (0.50 mL) were added Potassium phosphate tribasic (790 mg, 3.72 mmol, 3.0 eq), Potassium trifluoro (vinyl) borate (1-) (332 mg, 2.48 mmol, 2.0 eq), and 1,1′— Bis(diphenylphosphino) ferrocene-palladium (II) dichloride (90.8 mg, 124 μmol, 0.1 eq). The mixture was stirred under N₂ at 80° C. for 16 h. After cooled to room temperature, the reaction mixture was quenched with water (20 mL) and extracted with EA (20 mL×3). The organic layer was washed with brine (50 mL), dried over anhydrous Na₂SO₄, filtered and concentrated under reduced pressure. The crude product was purified by flash column chromatography on silica gel (ethyl acetate in petroleum ether, 13% v/v) to obtain 5-chloro-3-((2-(trimethylsilyl)ethoxy)methyl)-7-vinyl-3H-imidazo[4,5-b]pyridine (390 mg, yield 99%) as a yellow solid. LC-MS (ESI): mass calced for: C₁₄H₂OClN₃OSi, 309.11; m/z found, 310.9 [M+H]⁺. ¹H NMR (400 MHZ, DMSO-d₆) δ 8.78 (d, J=9.8 Hz, 1H), 7.76 (s, 1H), 6.99-6.90 (m, 1H), 6.46-6.42 (m, 1H), 5.78-5.71 (m, 3H), 3.73-3.70 (m, 2H), 1.00-0.93 (m, 2H), 0.00 (s, 9H).

### Step C: 5-chloro-3-((2-(trimethylsilyl)ethoxy)methyl)-3H-imidazo[4,5-b]pyridine-7-carbaldehyde

Ozone (O₃) was continuously bubbled into a solution of 5-chloro-3-((2-(trimethylsilyl)ethoxy)methyl)-7-vinyl-3H-imidazo[4,5-b]pyridine (210 mg, 678 μmol, 1.0 eq) in DCM (20.0 mL) at −70° C. for 1 h. After removed excess O₃ with N₂, dimethyl sulfide (5.0 mL) was added dropwise to above mixture and the mixture was stired for 30 min. After evaporation, the crude product was purified by Prep-TLC(PE/EA=1/1 v/v) to obtain 5-chloro-3-((2-(trimethylsilyl)ethoxy)methyl)-3H-imidazo[4,5-b]pyridine-7-carbaldehyde (70 mg, yield 33%) as a yellow oil. LC-MS (ESI): mass calced for: C₁₃H₁₈ClN₃O₂Si, 311.09; m/z found, 312.1 [M+H]⁺.

### Intermediate 46:3-isopropylpyrrolidine

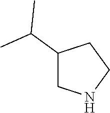

### Step A: benzyl 3-hydroxy-4-isopropylpyrrolidine-1-carboxylate

To a solution of benzyl 6-oxa-3-azabicyclo[3.1.0]hexane-3-carboxylate (2.30 g, 10.5 mmol, 1.0 eq) and CuBr-Me₂S (431 mg, 2.10 mmol, 0.2 eq) in THF (50.0 mL) was added dropwise isopropylmagnesium bromide (2 M in THF) (23.6 mL, 47.2 mmol, 4.5 eq) under N₂ at −20° C. The mixture solution was stirred at −20° C. for 4 h. The mixture solution was quenched with saturated aqueous NH₄Cl solution (50 mL) and extracted with EA (100 mL×3). The combined organic layers were washed with brine (100 mL), dried over anhydrous Na₂SO₄, filtered and concentrated to give a crude product. The crude product was purified by flash column chromatography on silica gel (PE/EA=5/1 v/v) to afford benzyl 3-hydroxy-4-isopropylpyrrolidine-1-carboxylate (1.80 g, yield 65%) as a colorless oil. LC-MS (ESI): mass calcd. for C₁₅H₂₁NO₃, 263.1; m/z found, 264.2 [M+H]⁺. ¹H NMR (400 MHZ, DMSO-d₆) δ 7.37-7.32 (m, 5H), 5.05-5.02 (m, 3H), 3.60-3.42 (m, 2H), 3.11-2.96 (m, 3H), 1.77-1.72 (m, 1H), 1.60-1.54 (m, 1H), 0.95-0.92 (m, 3H), 0.86-0.82 (m, 3H).

### Step B: benzyl 3-isopropyl-4-(((methylthio) carbonothioyl)oxy) pyrrolidine-1-carboxylate

To a solution of benzyl 3-hydroxy-4-isopropylpyrrolidine-1-carboxylate (1.80 g, 6.84 mmol, 1.0 eq) in THF (50.0 mL) was added NaH (60% suspend in oil) (0.60 g, 15.0 mmol, 2.2 eq) at 0° C. and the mixture was stirred at 25° C. for 2 h. Then CS2 (1.67 g, 1.32 mL, 21.9 mmol, 3.2 eq) was added to above mixture and the mixture was stirred at 25° C. for 1 h. MeI (2.04 g, 898 μL, 14.4 mmol, 2.1 eq) was added and the resulting mixture was stirred at 25° C. for 10 h. The mixture was quenched with saturated aqueous NaHCO₃ solution (50 mL) and extracted with EA (100 mL×3). The combined organic layers were washed with brine (100 mL), dried over anhydrous Na₂SO₄, filtered and concentrated under reduced pressure. The residue was purified by flash column chromatography on silica gel (PE/EA=8/1 v/v) to afford product benzyl 3-isopropyl-4-(((methylthio) carbonothioyl)oxy) pyrrolidine-1-carboxylate (1.50 g, yield 62%) as a colorless oil. LC-MS (ESI): mass calcd. for C₁₇H₂₃NO₃S₂, 353.1; m/z found, 354.3 [M+H]⁺.

### Step C: benzyl 3-isopropylpyrrolidine-1-carboxylate

To a solution of benzyl 3-isopropyl-4-(((methylthio) carbonothioyl)oxy) pyrrolidine-1-carboxylate (1.50 g, 4.24 mmol, 1.0 eq) in toluene (35.0 mL) were added AIBN (139 mg, 849 μmol, 0.2 eq) and Tributyltin hydride (2.47 g, 2.30 mL, 8.49 mmol, 2.0 eq) under N₂ at room temperature. The mixture solution was stirred at 85° C. for 12 h. After cooled to room temperature, the mixture solution was quenched with saturated aqueous KF solution (100 mL) and stirred for 2 h. The mixture was extracted with EA (50 mL×3). The combined organic layers were dried over anhydrous Na₂SO₄, filtered and concentrated under reduced pressure. The residue was purified by flash column chromatography on silica gel (PE/EA=10/1 v/v) to afford product benzyl 3-isopropylpyrrolidine-1-carboxylate (700 mg, yield 66%) as a colorless oil. LC-MS (ESI): mass calcd. for C₁₅H₂₁NO₂, 247.2; m/z found, 248.3 [M+H]⁺.

### Step D: 3-isopropylpyrrolidine

To a solution of benzyl 3-isopropylpyrrolidine-1-carboxylate (700 mg, 2.83 mmol, 1.0 eq) in MeOH (10.0 mL) were added 10% Pd/C(301 mg, 283 μmol) and con. HCl (1.00 mL). The mixture solution was stirred under H₂ (1 atm) at 25° C. for 12 h. After filtration, the filtrate was concentrated under reduced pressure to give product 3-isopropylpyrrolidine hydrochloride (285 mg, yield 89%) as a colorless oil. LC-MS (ESI): mass calcd. for C₇H₁₅N, 113.1; m/z found, 114.2 [M+H]⁺.

### Intermediate 47:1-benzyl-6-chloro-1H-pyrrolo[2,3-b]pyridine-4-carbaldehyde

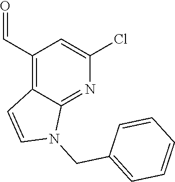 [SMILES: O=Cc1cc(Cl)nc2c1ccn2Cc1ccccc1]

### Step A: methyl 1-benzyl-6-chloro-1H-pyrrolo[2,3-b]pyridine-4-carboxylate

To a stirred solution of methyl 6-chloro-1H-pyrrolo[2,3-b]pyridine-4-carboxylate (300 mg, 1.42 mmol, 1.0 eq) and (bromomethyl)benzene (292 mg, 1.71 mmol, 1.2 eq) in DMF (5.00 mL) was added Cesium carbonate (928 mg, 2.85 mmol, 2.0 eq). The reaction mixture was stirred under nitrogen atmosphere at 80° C. for 1 h. After cooled to room temperature, the reaction mixture was diluted with water (15 mL) and extracted with EtOAc (15 mL×3). The combined organic phases were washed with brine (15 mL×4), dried over anhydrous sodium sulfate, filtered and concentrated under reduced pressure. The residue was purified by flash column chromatography on silica gel (PE/EtOAc=4/1 v/v) to obtain methyl 1-benzyl-6-chloro-1H-pyrrolo[2,3-b]pyridine-4-carboxylate (400 mg, yield 93%) as a green solid. LC-MS (ESI): mass calced for: C₁₆H₁₃ClN₂O₂, 300.07; m/z found, 301.1 [M+H]⁺.

### Step B: (1-benzyl-6-chloro-1H-pyrrolo[2,3-b]pyridin-4-yl) methanol

To a stirred solution of methyl 1-benzyl-6-chloro-1H-pyrrolo[2,3-b]pyridine-4-carboxylate (400 mg, 1.33 mmol, 1.0 eq) in THF (5.00 mL) was added Aluminum lithium hydride (50.5 mg, 1.33 mmol, 1.0 eq) at 0° C. and the reaction mixture was stirred under nitrogen atmosphere at 0° C. for 10 min. Then the reaction mixture was quenched with aqueous NaOH solution (1 N, 1 mL) and extracted with EtOAc (15 mL×3). The combined organic phase was washed with brine (15 mL), dried over anhydrous sodium sulfate, and filtered. The filtrate was concentrated under reduced pressure to obtain (1-benzyl-6-chloro-1H-pyrrolo[2,3-b]pyridin-4-yl) methanol (350 mg, yield 96%) as a red oil. LC-MS (ESI): mass calced for: C₁₅H₁₃ClN₂O, 272.07; m/z found, 273.1 [M+H]⁺.

### Step C: 1-benzyl-6-chloro-1H-pyrrolo[2,3-b]pyridine-4-carbaldehyde

To a stirred solution of (1-benzyl-6-chloro-1H-pyrrolo[2,3-b]pyridin-4-yl) methanol (350 mg, 1.28 mmol, 1.0 eq) in DMSO (5.00 mL) was added IBX (Purity 45˜55%) (1.08 g, 3.85 mmol, 3.0 eq) in portions. The reaction mixture was stirred at 30° C. for 30 min. The reaction mixture was quenched with saturated aqueous Na₂S₂O₃ solution (30 mL) and extracted with EtOAc (20 mL×3). The combined organic phase was washed with brine (20 mL×4), dried over anhydrous sodium sulfate, filtered and concentrated under reduced pressure. The residue was purified by flash column chromatography on silica gel (PE/EtOAc=4/1 v/v) to obtain 1-benzyl-6-chloro-1H-pyrrolo[2,3-b]pyridine-4-carbaldehyde (200 mg, yield 57%) as a yellow solid. LC-MS (ESI): mass calced for: C₁₅H₁₁ClN₂O, 270.06; m/z found, 271.0 [M+H]⁺.

### Intermediate 48:1-(but-2-yn-1-yl)-6-chloro-1H-pyrrolo[2,3-b]pyridine-4-carbaldehyde

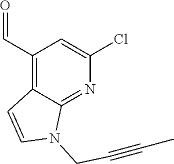 [SMILES: CC#CCn1ccc2c(C=O)cc(Cl)nc21]

### Step A: methyl 1-(but-2-yn-1-yl)-6-chloro-1H-pyrrolo[2,3-b]pyridine-4-carboxylate

To a stirred solution of methyl 6-chloro-1H-pyrrolo[2,3-b]pyridine-4-carboxylate (300 mg, 1.42 mmol, 1.0 eq) and 1-bromobut-2-yne (227 mg, 1.71 mmol, 1.2 eq) in DMF (5.00 mL) at room temperature was added Cesium carbonate (928 mg, 2.85 mmol, 2.0 eq). The reaction mixture was stirred under nitrogen atmosphere at 75° C. for 1 h. After cooled to room temperature, the reaction mixture was diluted with water (15 mL) and extracted with EtOAc (15 mL×3). The combined organic phases were washed with brine (15 mL×4), dried over anhydrous sodium sulfate, filtered and concentrated under reduced pressure. The residue was purified by flash column chromatography on silica gel (PE/EtOAc=4/1 v/v) to obtain methyl 1-(but-2-yn-1-yl)-6-chloro-1H-pyrrolo[2,3-b]pyridine-4-carboxylate (368 mg, yield 98%) as a green oil. LC-MS (ESI): mass calced for: C₁₃H₁₁ClN₂O₂, 262.05; m/z found, 263.0 [M+H]⁺.

### Step B: (1-(but-2-yn-1-yl)-6-chloro-1H-pyrrolo[2,3-b]pyridin-4-yl) methanol

To a stirred solution of methyl 1-(but-2-yn-1-yl)-6-chloro-1H-pyrrolo[2,3-b]pyridine-4-carboxylate (370 mg, 1.41 mmol, 1.0 eq) in dry THF (5.00 mL) was added Aluminum lithium hydride (53.5 mg, 1.41 mmol, 1.0 eq) at 0° C. and the reaction mixture was stirred under nitrogen atmosphere at 0° C. for 10 min. Then the reaction mixture was quenched with aqueous NaOH solution (1 N, 1 mL) and extracted with EtOAc (15 mL×3). The combined organic phase was washed with brine (15 mL), dried over anhydrous sodium sulfate, and filtered. The filtrate was concentrated under reduced pressure to obtain (1-(but-2-yn-1-yl)-6-chloro-1H-pyrrolo[2,3-b]pyridin-4-yl) methanol (300 mg, yield 91%) as a gray solid. The crude product was directly used in next step without further purification. LC-MS (ESI): mass calced for: C₁₂H₁₁ClN₂O, 234.06; m/z found, 235.1 [M+H]⁺.

### Step C: 1-(but-2-yn-1-yl)-6-chloro-1H-pyrrolo[2,3-b]pyridine-4-carbaldehyde

To a stirred solution of (1-(but-2-yn-1-yl)-6-chloro-1H-pyrrolo[2,3-b]pyridin-4-yl) methanol (300 mg, 1.28 mmol, 1.0 eq) in DMSO (5.00 mL) was added IBX (Purity 45˜55%) (1.07 g, 3.84 mmol, 3.0 eq). The reaction mixture was stirred at 30° C. for 30 min. The reaction mixture was quenched with saturated aqueous Na₂S₂O₃ solution (30 mL) and extracted with EtOAc (20 mL×3). The combined organic phase was washed with brine (20 mL×4), dried over anhydrous sodium sulfate, filtered and concentrated under reduced pressure. The residue was purified by flash column chromatography on silica gel (PE/EtOAc=5/1 v/v) to obtain 1-(but-2-yn-1-yl)-6-chloro-1H-pyrrolo[2,3-b]pyridine-4-carbaldehyde (200 mg, yield 67%) as a yellow solid. LC-MS (ESI): mass calced for: C₁₂H₉ClN₂O, 232.04; m/z found, 233.0 [M+H]⁺.

### Intermediate 49:2-(6-chloro-4-formyl-1H-pyrrolo[2,3-b]pyridin-1-yl)-N,N-dimethylacetamide

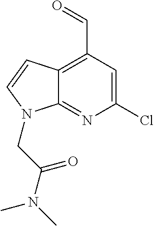 [SMILES: CN(C)C(=O)Cn1ccc2c(C=O)cc(Cl)nc21]

### Step A: methyl 6-chloro-1-(2-(dimethylamino)-2-oxoethyl)-1H-pyrrolo[2,3-b]pyridine-4-carboxylate

To a solution of methyl 6-chloro-1H-pyrrolo[2,3-b]pyridine-4-carboxylate (300 mg, 1.42 mmol, 1.0 eq) and 2-bromo-N,N-dimethylacetamide (236 mg, 1.42 mmol, 1.0 eq) in DMF (3.0 mL) was added Cs₂CO₃ (928 mg, 2.9 mmol, 2.0 eq) at 25° C. The mixture was stirred at 50° C. for 5 h. The reaction mixture was diluted with EtOAc (5 mL), washed with saturated aqueous NH₄Cl solution (10 mL×3). The organic phase was dried over anhydrous Na₂SO₄, filtered and concentrated under reduced pressure. The residue was purified by flash column chromatography on silica gel (PE/EA=1/1 v/v) to give methyl 6-chloro-1-(2-(dimethylamino)-2-oxoethyl)-1H-pyrrolo[2,3-b]pyridine-4-carboxylate (300 mg, yield 71%) as a yellow solid. LC-MS (ESI): mass calcd. for C₁₃H₁₄ClN₃O₃, 295.1; m/z found, 296.1 [M+H]⁺.

### Step B: 2-(6-chloro-4-(hydroxymethyl)-1H-pyrrolo[2,3-b]pyridin-1-yl)-N,N-dimethylacetamide

To a solution of methyl 6-chloro-1-(2-(dimethylamino)-2-oxoethyl)-1H-pyrrolo[2,3-b]pyridine-4-carboxylate (300 mg, 1.0 mmol, 1.0 eq) in THF (5.0 mL) was added LiBH₄ (33% W.t. in THF) (7.33 g, 25.8 mmol, 25.0 eq) at 0° C. The reaction mixture was stirred at room temperature for 2 h. The reaction mixture was quenched with water (20 mL) and extracted with EA (20 mL×3). The organic layer was washed with brine (30 mL), dried over anhydrous Na₂SO₄, filtered and concentrated under reduced pressure. The residue was purified by flash column chromatography on silica gel (PE/EA=2/1 v/v) to give 2-(6-chloro-4-(hydroxymethyl)-1H-pyrrolo[2,3-b]pyridin-1-yl)-N,N-dimethylacetamide (100 mg, yield 37%) as a yellow oil. LC-MS (ESI): mass calcd. for C₁₂H₁₄ClN₃O₂, 267.1; m/z found, 268.1 [M+H]⁺.

### Step C: 2-(6-chloro-4-formyl-1H-pyrrolo[2,3-b]pyridin-1-yl)-N,N-dimethylacetamide

To a solution of 2-(6-chloro-4-(hydroxymethyl)-1H-pyrrolo[2,3-b]pyridin-1-yl)-N,N-dimethylacetamide (100 mg, 374 μmol, 1.0 eq) in DMSO (5.0 mL) was added IBX (314 mg, 1.1 mmol, 3.0 eq) at 25° C. The reaction mixture was stirred at room temperature for 20 min. The reaction mixture was quenched with saturated aqueous NaHCO₃ solution (10 mL), saturated aqueous Na₂S₂O₃ solution (10 mL), and extracted with DCM (10 mL×3). The organic layer was washed with brine (20 mL), dried over anhydrous Na₂SO₄, filtered and concentrated under reduced pressure. The residue was purified by flash column chromatography on silica gel (PE/EA=1/1 v/v) to give 2-(6-chloro-4-formyl-1H-pyrrolo[2,3-b]pyridin-1-yl)-N,N-dimethylacetamide (90 mg, yield 91%) as a yellow oil. LC-MS (ESI): mass calcd. for C₁₂H₁₂ClN₃O₂, 265.1; m/z found, 266.1 [M+H]⁺.

### Intermediate 50:2-(6-chloro-4-formyl-1H-pyrrolo[2,3-b]pyridin-1-yl)-N-methylacetamide

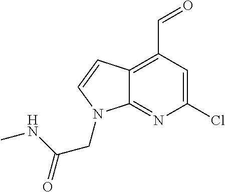 [SMILES: CNC(=O)Cn1ccc2c(C=O)cc(Cl)nc21]

### Step A: methyl 6-chloro-1-(2-(methylamino)-2-oxoethyl)-1H-pyrrolo[2,3-b]pyridine-4-carboxylate

To a solution of methyl 6-chloro-1H-pyrrolo[2,3-b]pyridine-4-carboxylate (300 mg, 1.4 mmol, 1.0 eq) and 2-bromo-N-methylacetamide (216 mg, 1.4 mmol, 1.0 eq) in DMF (10 mL) was added Cs₂CO₃ (928 mg, 2.8 mmol, 2.0 eq) at 25° C., the mixture was stirred at 50° C. for 3 h. The reaction mixture was cooled to room temperature, quenched with H₂O (20 mL), and extracted with EA (30 mL×3). The organic layer was washed with brine (30 mL×4), dried over anhydrous Na₂SO₄, filtered and concentrated under reduced pressure. The residue was purified by flash column chromatography on silica gel (PE/EA=2/1 v/v) to give methyl 6-chloro-1-(2-(methylamino)-2-oxoethyl)-1H-pyrrolo[2,3-b]pyridine-4-carboxylate (300 mg, yield 75%) as a yellow solid. LC-MS (ESI): mass calcd. for C₁₂H₁₂ClN₃O₃, 281.06; m/z found, 282.1 [M+H]⁺.

### Step B: 2-(6-chloro-4-(hydroxymethyl)-1H-pyrrolo[2,3-b]pyridin-1-yl)-N-methylacetamide

To a solution of methyl 6-chloro-1-(2-(methylamino)-2-oxoethyl)-1H-pyrrolo[2,3-b]pyridine-4-carboxylate (300 mg, 1.1 mmol, 1.0 eq) in THF (10.0 mL) was added dropwise LiBH₄ (33% W.t in THF) (4.13 mL, 10.6 mmol, 10.0 eq) at 25° C. and the mixture was stirred under N₂ at 25° C. for 1 h. The mixture was quenched with saturated aqueous NH₄Cl solution (30 mL) and extracted with EA (30 mL×3). The organic layer was washed with brine (30 mL), dried over anhydrous Na₂SO₄, filtered and concentrated in vacuum. The residue was purified by flash column chromatography on silica gel (PE/EA=1/1 v/v) to give 2-(6-chloro-4-(hydroxymethyl)-1H-pyrrolo[2,3-b]pyridin-1-yl)-N-methylacetamide (200 mg, yield 74%) as a yellow solid. LC-MS (ESI): mass calcd. for C₁₁H₁₂ClN₃O₂, 253.1; m/z found, 254.1 [M+H]⁺.

### Step C: 2-(6-chloro-4-formyl-1H-pyrrolo[2,3-b]pyridin-1-yl)-N-methylacetamide

To a solution of 2-(6-chloro-4-(hydroxymethyl)-1H-pyrrolo[2,3-b]pyridin-1-yl)-N-methylacetamide (100 mg, 394 μmol, 1.0 eq) in DMSO (2.0 mL) was added IBX (Purity 45% W.t) (331 mg, 1.2 mmol, 3.0 eq) at 25° C. The mixture was stirred at 25° C. for 1 h. The reaction mixture was quenched with saturated aqueous Na₂S₂O₃ solution (20 mL) and extracted with EA (15 mL×3). The organic layer was washed with brine (10 mL×4), dried over anhydrous Na₂SO₄, filtered and concentrated in vacuum. The residue was purified by Prep-TLC(PE/EA=1/1 v/v) to give 2-(6-chloro-4-formyl-1H-pyrrolo[2,3-b]pyridin-1-yl)-N-methylacetamide (90.0 mg, yield 91%) as a yellow solid. LC-MS (ESI): mass calcd. for C₁₁H₁₀ClN₃O₂, 251.1; m/z found, 252.1 [M+H]⁺.

### Intermediate 51:6-chloro-1-cyclopentyl-1H-pyrrolo[2,3-b]pyridine-4-carbaldehyde

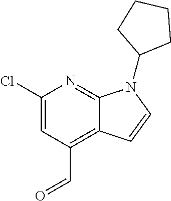 [SMILES: O=Cc1cc(Cl)nc2c1ccn2C1CCCC1]

### Step A: methyl 6-chloro-1-cyclopentyl-1H-pyrrolo[2,3-b]pyridine-4-carboxylate

To a solution of methyl 6-chloro-1H-pyrrolo[2,3-b]pyridine-4-carboxylate (300 mg, 1.4 mmol, 1.0 eq) and Cs₂CO₃ (928 mg, 2.9 mmol, 2.0 eq) in DMF (6.0 mL) was added iodocyclopentane (335 mg 1.7 mmol, 1.2 eq) at 20° C. The reaction mixture was stirred at 70° C. for 5 h. After cooled to room temperature, the reaction mixture was quenched with water (10 mL) and extracted with EA (10 mL×3). The organic layer was washed with brine (10 mL×3), dried over anhydrous Na₂SO₄, filtered and concentrated under reduced pressure. The residue was purified by flash column chromatography on silica gel (PE/EA=20/1 v/v) to give methyl 6-chloro-1-cyclopentyl-1H-pyrrolo[2,3-b]pyridine-4-carboxylate (310 mg, yield 78%) as a yellow oil. LC-MS (ESI): mass calcd. for C₁₄H₁₅ClN₂O₂, 278.1; m/z found, 279.1 [M+H]⁺.

### Step B: (6-chloro-1-cyclopentyl-1H-pyrrolo[2,3-b]pyridin-4-yl) methanol

To a solution of methyl 6-chloro-1-cyclopentyl-1H-pyrrolo[2,3-b]pyridine-4-carboxylate (300 mg, 1.1 mmol, 1.0 eq) in THF (5.0 mL) was added LAH (81.7 mg, 2.2 mmol, 2.0 eq) at 0° C. The reaction mixture was stirred at 0° C. for 2 min. The reaction mixture was quenched with water (10 mL) and extracted with EA (10 mL×3). The organic layer was washed with brine (10 mL), dried over anhydrous Na₂SO₄, filtered and concentrated under reduced pressure. The residue was purified by flash column chromatography on silica gel (PE/EA=2/1 v/v) to give (6-chloro-1-cyclopentyl-1H-pyrrolo[2,3-b]pyridin-4-yl) methanol (260 mg, yield 96%) as a yellow oil. LC-MS (ESI): mass calcd. for C₁₃H₁₅ClN₂O, 250.1; m/z found, 251.1 [M+H]⁺.

### Step C: 6-chloro-1-cyclopentyl-1H-pyrrolo[2,3-b]pyridine-4-carbaldehyde

To a solution of (6-chloro-1-cyclopentyl-1H-pyrrolo[2,3-b]pyridin-4-yl) methanol (270 mg, 1.1 mmol, 1.0 eq) in DMSO (5.0 mL) was added IBX (purity 45%˜55%) (905 mg, 2.2 mmol, 2.0 eq) at 25° C. The reaction mixture was stirred at room temperature for 20 min. The reaction mixture was quenched with water (20 mL) and extracted with DCM (10 mL×3). The organic layer was washed with brine (30 mL×4), dried over anhydrous Na₂SO₄, filtered and concentrated under reduced pressure. The residue was purified by flash column chromatography on silica gel (PE/EA=20/1 v/v) to give 6-chloro-1-cyclopentyl-1H-pyrrolo[2,3-b]pyridine-4-carbaldehyde (250 mg, yield 93%) as a yellow oil. LC-MS (ESI): mass calcd. for C₁₃H₁₃ClN₂O, 248.1; m/z found, 249.1 [M+H]⁺.

### Intermediate 52:5-chloro-1-((2-(trimethylsilyl)ethoxy)methyl)-1H-pyrrolo[3,2-b]pyridine-7-carbaldehyde

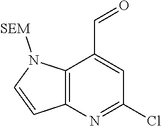 [SMILES: C[Si](C)(C)CCOCn1ccc2nc(Cl)cc(C=O)c21]

### Step A: methyl 5-chloro-1-((2-(trimethylsilyl)ethoxy)methyl)-1H-pyrrolo[3,2-b]pyridine-7-carboxylate

To a solution of methyl 5-chloro-1H-pyrrolo[3,2-b]pyridine-7-carboxylate (400 mg, 1.90 mmol, 1.0 eq) in DMF (5.00 mL) was added NaH (60% suspend in oil) (68 mg, 2.85 mmol, 1.5 eq) at 0° C. After 30 min, (2-(chloromethoxy)ethyl)trimethylsilane (633 mg, 3.80 mmol, 2.0 eq) was added dropwise to above mixture and the mixture was stirred at room temperature for 1 h. The reaction mixture was quenched with saturated aqueous NH₄Cl solution (50 mL) and extracted with EA (50 mL×4). The organic layer was washed with brine (50 mL×4), dried over anhydrous Na₂SO₄, filtered and concentrated in vacuum. The crude product was purified by flash column chromatography on silica gel (PE/EA=2/1 v/v) to give methyl 5-chloro-1-((2-(trimethylsilyl)ethoxy)methyl)-1H-pyrrolo[3,2-b]pyridine-7-carboxylate (220 mg, yield 34%) as a colorless oily. LC-MS (ESI): mass calced for: C₁₅H₂₁ClN₂O₃Si, 340.10; m/z found, 341.1 [M+H]⁺.

### Step B: (5-chloro-1-((2-(trimethylsilyl)ethoxy)methyl)-1H-pyrrolo[3,2-b]pyridin-7-yl) methanol

To a solution of methyl 5-chloro-1-((2-(trimethylsilyl)ethoxy)methyl)-1H-pyrrolo[3,2-b]pyridine-7-carboxylate (220 mg, 645 μmol, 1.0 eq) in anhydrous THF (10 mL) was added LAH (49.0 mg, 1.29 mmol, 2.0 eq) in portions at 0° C. The reaction mixture was stirred at 0° C. for 20 min. The reaction mixture was slowly quenched with aqueous NaOH solution (1 N) (40 mL) and extracted with EtOAc (40 mL×3). The organic layer was washed with brine (40 mL×4), dried over anhydrous Na₂SO₄, filtered and concentrated under reduced pressure. The residue was purified by flash column chromatography on silica gel (PE/EA=1/1 v/v) to afford (5-chloro-1-((2-(trimethylsilyl)ethoxy)methyl)-1H-pyrrolo[3,2-b]pyridin-7-yl) methanol (180 mg, yield 89%) as a yellow solid. LC-MS (ESI): mass calced for: C₁₄H₂₁ClN₂O₂Si, 312.11; m/z found, 313.1 [M+H]⁺.

### Step C: 5-chloro-1-((2-(trimethylsilyl)ethoxy)methyl)-1H-pyrrolo[3,2-b]pyridine-7-carbaldehyde

To a solution of (5-chloro-1-((2-(trimethylsilyl)ethoxy)methyl)-1H-pyrrolo[3,2-b]pyridin-7-yl) methanol (180 mg, 575 μmol, 1.0 eq) in DMSO (5.00 mL) was added IBX (Purity 45% W.t) (483 mg, 1.73 mmol, 3.0 eq) at room temperature. The reaction mixture was stirred at room temperature for 1 h. The reaction mixture was quenched with water (5 mL) and extracted with EtOAc (15 mL×3). The organic layer was washed with brine (5 mL), dried over anhydrous Na₂SO₄, filtered and concentrated under reduced pressure. The residue was purified by Prep-TLC(DCM/MeOH=10/1 v/v) to give 5-chloro-1-((2-(trimethylsilyl)ethoxy)methyl)-1H-pyrrolo[3,2-b]pyridine-7-carbaldehyde (150 mg, yield 84%) as a yellow solid. LC-MS (ESI): mass calced for: C₁₄H₁₉ClN₂O₂Si, 310.09; m/z found, 311.1 [M+H]⁺

### Intermediate 53:6-chloro-1-cyclohexyl-1H-pyrrolo[2,3-b]pyridine-4-carbaldehyde

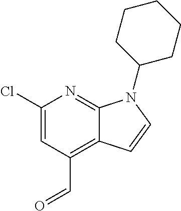 [SMILES: O=Cc1cc(Cl)nc2c1ccn2C1CCCCC1]

### Step A: methyl 6-chloro-1-cyclohexyl-1H-pyrrolo[2,3-b]pyridine-4-carboxylate

To a solution of methyl 6-chloro-1H-pyrrolo[2,3-b]pyridine-4-carboxylate (300 mg, 1.4 mmol, 1.0 eq), cyclohexanol (214 mg, 2.1 mmol, 1.5 eq), and PPh3 (560 mg, 2.1 mmol, 1.5 eq) in THF (10 mL) was added DIAD (432 mg, 2.1 mmol, 1.5 eq) at 0° C. The reaction mixture was stirred under N₂ at room temperature for 16 h. The reaction mixture was quenched with H₂O (20 mL) and extracted with EA (30 mL×3). The organic layer was washed with brine (30 mL), dried over Na₂SO₄, filtered and concentrated under reduced pressure. The residue was purified by flash column chromatography on silica gel (PE/EA=20/1 v/v) to give methyl 6-chloro-1-cyclohexyl-1H-pyrrolo[2,3-b]pyridine-4-carboxylate (180 mg, yield 43%) as a yellow oil. LC-MS (ESI): mass calcd. for C₁₅H₁₇ClN₂O₂, 292.1; m/z found, 293.1 [M+H]⁺.

### Step B: (6-chloro-1-cyclohexyl-1H-pyrrolo[2,3-b]pyridin-4-yl) methanol

To a solution of methyl 6-chloro-1-cyclohexyl-1H-pyrrolo[2,3-b]pyridine-4-carboxylate (180 mg, 615 μmol, 1.0 eq) in THF (5.0 mL) was added LAH (46.7 mg, 1.2 mmol, 2.0 eq) at 0° C. The reaction mixture was stirred at 0° C. for 5 min. The reaction mixture was quenched with H₂O (20 mL) and extracted with EA (30 mL×3). The organic layer was washed with brine (30 mL), dried over Na₂SO₄, and filtered. The filtrate was concentrated under reduced pressure. To give (6-chloro-1-cyclohexyl-1H-pyrrolo[2,3-b]pyridin-4-yl) methanol (150 mg, yield 92%) as a yellow oil, which was used directly in next step without further purification. LC-MS (ESI): mass calcd. for C₁₄H₁₇ClN₂O, 264.1; m/z found, 265.1 [M+H]⁺.

### Step C: 6-chloro-1-cyclohexyl-1H-pyrrolo[2,3-b]pyridine-4-carbaldehyde

To a solution of (6-chloro-1-cyclohexyl-1H-pyrrolo[2,3-b]pyridin-4-yl) methanol (150 mg, 567 μmol, 1.0 eq) in DMSO (5.0 mL) was added IBX (Purity 45%˜55%) (476 mg, 1.7 mmol, 3.0 eq) at 25° C. The reaction mixture was stirred at room temperature for 15 min. The reaction mixture was quenched with water (20 mL) and extracted with EA (30 mL×3). The organic layer was washed with brine (30 mL), dried over Na₂SO₄, filtered and concentrated under reduced pressure. The residue was purified by flash column chromatography on silica gel (PE/EA=20/1 v/v) to give 6-chloro-1-cyclohexyl-1H-pyrrolo[2,3-b]pyridine-4-carbaldehyde (120 mg, yield 81%) as a yellow oil. LC-MS (ESI): mass calcd. for C₁₄H₁₅ClN₂O, 262.1; m/z found, 263.1 [M+H]⁺.

### Intermediate 54: N,N-dimethyl-2-(pyrrolidin-3-yl) acetamide

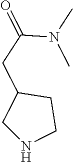

### Step A: tert-butyl 3-(2-(dimethylamino)-2-oxoethyl) pyrrolidine-1-carboxylate

To a solution of 2-(1-(tert-butoxycarbonyl) pyrrolidin-3-yl) acetic acid (500 mg, 2.18 mmol, 1.0 eq) in DMF (6.00 mL) were added DIPEA (846 mg, 6.54 mmol, 3.0 eq) and HATU (1.24 g, 3.27 mmol, 1.5 eq). Dimethylamine (108 mg, 2.40 mmol 1.1 eq) was added to above at 0° C. and the mixture was stirred at 30° C. for 16 h. The reaction solution was diluted with water (20 mL) and extracted with EA (10 mL×3). The organic layer was washed with brine (20 mL), dried over anhydrous Na₂SO₄, filtered and concentrated under reduced pressure to give crude product tert-butyl 3-(2-(dimethylamino)-2-oxoethyl) pyrrolidine-1-carboxylate (380 mg, yield 68%) as a colorless oily. LC-MS (ESI): mass calced for: C₁₃H₂₄N₂O₃, 256.35; m/z found, 257.3 [M+H]⁺. ¹H NMR (400 MHZ, DMSO-d₆) δ 3.47 (td, J=10.0, 6.2 Hz, 1H), 3.29 (dd, J=8.2, 3.6 Hz, 1H), 3.21-3.09 (m, 1H), 2.94 (s, 3H), 2.82-2.76 (m, 4H), 2.47-2.34 (m, 3H), 1.95 (s, 1H), 1.53-1.42 (m, 1H), 1.39 (s, 9H).

### Step B: N,N-dimethyl-2-(pyrrolidin-3-yl) acetamide

To a solution of tert-butyl 3-(2-(dimethylamino)ethyl) pyrrolidine-1-carboxylate (380 mg, 1.57 mmol, 1.0 eq) in 1,4-Dioxane (3.00 mL) was added HCl (4 N in dioxane) (8.00 mL, 32.0 mmol, 20.4 eq). The reaction mixture was stirred at 30° C. for 1 h. The mixture was concentrated under reduced pressure to give crude product N,N-dimethyl-2-(pyrrolidin-3-yl) ethan-1-amine hydrochloride (222 mg, yield 99%) as a white solid. LC-MS (ESI): mass calced for: C₈H₁₆N₂O, 156.23; m/z found, 157.2 [M+H]⁺.

### Intermediate 55: tert-butyl 3-(6-chloro-4-formyl-1H-pyrrolo[2,3-b]pyridin-1-yl) azetidine-1-carboxylate

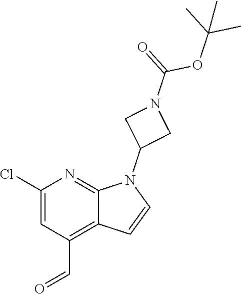 [SMILES: CC(C)(C)OC(=O)N1CC(n2ccc3c(C=O)cc(Cl)nc32)C1]

### Step A: methyl 1-(1-(tert-butoxycarbonyl) azetidin-3-yl)-6-chloro-1H-pyrrolo[2,3-b]pyridine-4-carboxylate

To a solution of methyl 6-chloro-1H-pyrrolo[2,3-b]pyridine-4-carboxylate (300 mg, 1.42 mmol, 1.0 eq) and tert-butyl 3-bromoazetidine-1-carboxylate (841 mg, 3.56 mmol, 2.5 eq) in DMF (5.00 mL) were added Cs₂CO₃ (1.39 g, 4.27 mmol, 3.0 eq) and KI (47.3 mg, 285 μmol, 0.2 eq) at room temperature. The reaction mixture was stirred at 80° C. for 18 h. The crude mixture was cooled to room temperature, poured into ice-water (20 mL), and extracted with ethyl acetate (15 mL×3). The combined organic layers were washed with brine (40 mL×3), dried over anhydrous Na₂SO₄, filtered and concentrated under reduced pressure. The crude product was purified by flash column chromatography on silica gel (ethyl acetate in petroleum ether, 15% v/v) to afford methyl 1-(1-(tert-butoxycarbonyl) azetidin-3-yl)-6-chloro-1H-pyrrolo[2,3-b]pyridine-4-carboxylate (380 mg, yield 72%) as a white solid. LC-MS (ESI): mass calced for: C₁₇H₂OClN₃O₄ 365.81; m/z found, 366.2 [M+H]⁺. ¹H NMR (400 MHZ, DMSO-d₆) δ 8.13 (d, J=3.6 Hz, 1H), 7.61 (s, 1H), 6.96 (d, J=3.6 Hz, 1H), 5.62-5.54 (m, 1H), 4.39 (t, J=8.4 Hz, 2H), 4.27 (d, J=5.6 Hz, 2H), 3.97 (s, 3H), 1.44 (s, 9H).

### Step B: tert-butyl 3-(6-chloro-4-(hydroxymethyl)-1H-pyrrolo[2,3-b]pyridin-1-yl) azetidine-1-carboxylate

To a solution of methyl 1-(1-(tert-butoxycarbonyl) azetidin-3-yl)-6-chloro-1H-pyrrolo[2,3-b]pyridine-4-carboxylate (350 mg, 957 μmol, 1.0 eq) in THF (10.0 mL) was added LiAlH₄ (36.3 mg, 957 μmol, 1.0 eq) at 0° C. The reaction mixture was stirred under N₂ atmosphere at 0° C. for 10 min. The reaction mixture was quenched with Na₂SO₄·10H₂O (500 mg) at 0° C. and stirred for 10 min. After filtration, the filtrate was concentrated in vacuum to afford a crude product tert-butyl 3-(6-chloro-4-(hydroxymethyl)-1H-pyrrolo[2,3-b]pyridin-1-yl) azetidine-1-carboxylate (380 mg, yield 118%) as a green oil. The crude product was directly used in next step without further purification. LC-MS (ESI): mass calced for: C16H20ClN303 337.8; m/z found, 338.2 [M+H]⁺.

### Step C: tert-butyl 3-(6-chloro-4-formyl-1H-pyrrolo[2,3-b]pyridin-1-yl) azetidine-1-carboxylate

To a solution of tert-butyl 3-(6-chloro-4-(hydroxymethyl)-1H-pyrrolo[2,3-b]pyridin-1-yl) azetidine-1-carboxylate (390 mg, 1.15 mmol, 1.0 eq) in DMSO (5.00 mL) was added IBX (808 mg, 2.89 mmol, 2.5 eq). The resulting mixture was stirred at 30° C. for 30 min. The reaction mixture was quenched with saturated aqueous NaHCO₃ solution (15 mL) and filtered. The filtrate was diluted with water (15 mL) and extracted with EA (15 ml×3). The combined organic layers were washed with brine (30 mL×3), dried over anhydrous Na₂SO₄, filtered and concentrated under reduced pressure. The residue was purified by flash column chromatography on silica gel (ethyl acetate in petroleum ether, 22% v/v) to afford tert-butyl 3-(6-chloro-4-formyl-1H-pyrrolo[2,3-b]pyridin-1-yl) azetidine-1-carboxylate (190 mg, yield 49%) as a yellow solid. LC-MS (ESI): mass calced for: C16H18ClN3O3 335.79; m/z found, 358.2 [M+23]⁺. ¹H NMR (400 MHZ, DMSO-d₆) δ 10.30 (s, 1H), 8.18 (d, J=3.6 Hz, 1H), 7.77 (s, 1H), 7.10 (d, J=3.6 Hz, 1H), 5.63-5.56 (m, 1H), 4.39 (t, J=8.4 Hz, 2H), 4.27 (s, 2H), 1.44 (s, 9H).

### Intermediate 56:6-chloro-1-(tetrahydrofuran-3-yl)-1H-pyrrolo[2,3-b]pyridine-4-carbaldehyde

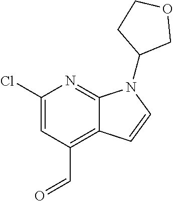 [SMILES: O=Cc1cc(Cl)nc2c1ccn2C1CCOC1]

### Step A: methyl 6-chloro-1-(tetrahydrofuran-3-yl)-1H-pyrrolo[2,3-b]pyridine-4-carboxylate

To a solution of methyl 6-chloro-1H-pyrrolo[2,3-b]pyridine-4-carboxylate (300 mg, 1.4 mmol, 1.0 eq) and Cs₂CO₃ (928 mg, 2.9 mmol, 2.0 eq) in DMF (6.0 mL) was added 3-iodotetrahydrofuran (423 mg, 2.1 mmol, 1.5 eq) at 20° C. The reaction mixture was stirred at 80° C. for 16 h. After cooled to room temperature, the reaction mixture was quenched with water (10 mL) and extracted with EA (10 mL×3). The organic layer was washed with brine (10 mL×4), dried over anhydrous Na₂SO₄, filtered and concentrated under reduced pressure. The residue was purified by flash column chromatography on silica gel (PE/EA=2/1 v/v) to give methyl 6-chloro-1-(tetrahydrofuran-3-yl)-1H-pyrrolo[2,3-b]pyridine-4-carboxylate (180 mg, yield 45%) as a yellow oil. LC-MS (ESI): mass calcd. for C₁₃H₁₃ClN₂O₃, 280.1; m/z found, 281.1 [M+H]⁺.

### Step B: (6-chloro-1-(tetrahydrofuran-3-yl)-1H-pyrrolo[2,3-b]pyridin-4-yl) methanol

To a solution of methyl 6-chloro-1-(tetrahydrofuran-3-yl)-1H-pyrrolo[2,3-b]pyridine-4-carboxylate (180 mg, 641 μmol, 1.0 eq) in THF (5.0 mL) was added LiAlH₄ (24.3 mg, 641 μmol, 1.0 eq) at 0° C. The reaction mixture was stirred at 0° C. for 5 min. The reaction mixture was quenched with water (10 mL) and extracted with EA (10 mL×3). The organic layer was washed with brine (10 mL), dried over Na₂SO₄, filtered and concentrated under reduced pressure to give (6-chloro-1-(tetrahydrofuran-3-yl)-1H-pyrrolo[2,3-b]pyridin-4-yl) methanol (160 mg, yield 99%) as a yellow oil. The crude product was directly used in next step without further. LC-MS (ESI): mass calcd. for C₁₂H₁₃ClN₂O₂, 252.1; m/z found, 253.1 [M+H]⁺.

### Step C: 6-chloro-1-(tetrahydrofuran-3-yl)-1H-pyrrolo[2,3-b]pyridine-4-carbaldehyde

To a solution of (6-chloro-1-(tetrahydrofuran-3-yl)-1H-pyrrolo[2,3-b]pyridin-4-yl) methanol (180 mg, 712 μmol, 1.0 eq) in DMSO (5.0 mL) was added IBX (purity 45%˜55%) (598 mg, 2.1 mmol, 3.0 eq) at 25° C. The reaction mixture was stirred at room temperature for 20 min. The reaction mixture was quenched with water (20 mL) and extracted with DCM (10 mL×3). The organic layer was washed with brine (30 mL×4), dried over anhydrous Na₂SO₄, filtered and concentrated under reduced pressure. The residue was purified by flash column chromatography on silica gel (PE/EA=5/1 v/v) to give 6-chloro-1-(tetrahydrofuran-3-yl)-1H-pyrrolo[2,3-b]pyridine-4-carbaldehyde (160 mg, yield 90%) as a yellow oil. LC-MS (ESI): mass calcd. for C₁₂H₁₁ClN₂O₂, 250.1; m/z found, 251.1 [M+H]⁺.

### Intermediate 57:6-chloro-1-(tetrahydro-2H-pyran-4-yl)-1H-pyrrolo[2,3-b]pyridine-4-carbaldehyde

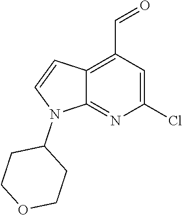 [SMILES: O=Cc1cc(Cl)nc2c1ccn2C1CCOCC1]

### Step A: methyl 6-chloro-1-(tetrahydro-2H-pyran-4-yl)-1H-pyrrolo[2,3-b]pyridine-4-carboxylate

To a mixture of tetrahydro-2H-pyran-4-yl methanesulfonate (513 mg, 2.8 mmol, 2.0 eq) and methyl 6-chloro-1H-pyrrolo[2,3-b]pyridine-4-carboxylate (300 mg, 1.4 mmol, 1.0 eq) in DMF (10.0 mL) was added K₂CO₃ (394 mg, 2.8 mmol, 2.0 eq) at 25° C. The mixture was stirred under N₂ at 100° C. for 16 h. After cooled to room temperature, the reaction mixture was quenched with water (20 mL) and extracted with EA (30 mL×3). The organic layer was washed with brine (30 mL×4), dried over anhydrous Na₂SO₄, filtered and concentrated under reduced pressure. The residue was purified by flash column chromatography on silica gel (PE/EA=3/1 v/v) to give methyl 6-chloro-1-(tetrahydro-2H-pyran-4-yl)-1H-pyrrolo[2,3-b]pyridine-4-carboxylate (150 mg, yield 36%) as a yellow solid. LC-MS (ESI): mass calcd. for C₁₄H₁₅ClN₂O₃, 294.1; m/z found, 295.1 [M+H]⁺.

### Step B: (6-chloro-1-(tetrahydro-2H-pyran-4-yl)-1H-pyrrolo[2,3-b]pyridin-4-yl) methanol

To a solution of methyl 6-chloro-1-(tetrahydro-2H-pyran-4-yl)-1H-pyrrolo[2,3-b]pyridine-4-carboxylate (150 mg, 509 μmol, 1.0 eq) in THF (5.0 mL) was added LiAlH₄ (38.6 mg, 1.0 mmol, 2.0 eq) at 0° C. under N₂. The reaction mixture was stirred at 0° C. for 5 min. The reaction mixture was quenched with water (20 mL) and extracted with EA (20 mL×3). The organic layer was washed with brine (30 mL), dried over anhydrous Na₂SO₄, filtered and concentrated under reduced pressure. The residue was purified by flash column chromatography on silica gel (PE/EA=20/1 v/v) to give (6-chloro-1-(tetrahydro-2H-pyran-4-yl)-1H-pyrrolo[2,3-b]pyridin-4-yl) methanol (120 mg, yield 88%) as a yellow solid. LC-MS (ESI): mass calcd. for C₁₃H₁₅ClN₂O₂, 266.1; m/z found, 267.1 [M+H]⁺.

### Step C: 6-chloro-1-(tetrahydro-2H-pyran-4-yl)-1H-pyrrolo[2,3-b]pyridine-4-carbaldehyde

To a solution of (6-chloro-1-(tetrahydro-2H-pyran-4-yl)-1H-pyrrolo[2,3-b]pyridin-4-yl) methanol (150 mg, 562 μmol, 1.0 eq) in DMSO (5.0 mL) was added IBX (Purity 45% W.t) (472 mg, 1.7 mmol, 3.0 eq) at room temperature and the reaction mixture was stirred at room temperature for 20 min. The reaction mixture was quenched with water (20 mL) and extracted with EA (30 mL×3). The organic layer was washed with brine (30 mL×3), dried over Na₂SO₄, filtered and concentrated under reduced pressure. The residue was purified by flash column chromatography on silica gel (PE/EA=10/1 v/v) to give 6-chloro-1-(tetrahydro-2H-pyran-4-yl)-1H-pyrrolo[2,3-b]pyridine-4-carbaldehyde (130 mg, yield 87%) as a yellow solid. LC-MS (ESI): mass calcd. for C₁₃H₁₃ClN₂O₂, 264.1; m/z found, 265.1 [M+H]⁺.

### Intermediate 58:5-chloro-1-isopropyl-1H-pyrrolo[3,2-b]pyridine-7-carbaldehyde

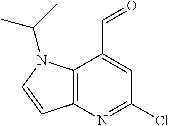

### Step A: methyl 5-chloro-1-isopropyl-1H-pyrrolo[3,2-b]pyridine-7-carboxylate

To a solution of methyl 5-chloro-1H-pyrrolo[3,2-b]pyridine-7-carboxylate (200 mg 950 μmol, 1.0 eq) and 2-iodopropane (323 mg, 1.90 mmol, 2.0 eq) in DMF (5.00 mL) was added Cs₂CO₃ (619 mg, 1.90 mmol, 2.0 eq) at room temperature. The reaction mixture was stirred at 80° C. for 16 h. The reaction mixture was cooled to room temperature, quenched with water (50 mL), and extracted with EtOAc (50 mL×3). The organic layer was washed with brine (50 mL×4), dried over anhydrous Na₂SO₄, filtered and concentrated under reduced pressure. The residue was purified by flash (ethyl acetate in petroleum ether, 15% v/v) to give methyl 5-chloro-1-isopropyl-1H-pyrrolo[3,2-b]pyridine-7-carboxylate (100 mg, yield 42%) as a white solid. LC-MS (ESI): mass calcd. for C₁₂H₁₃ClN₂O₂, 252.07; m/z found, 253.2 [M+H]⁺.

### Step B: (5-chloro-1-isopropyl-1H-pyrrolo[3,2-b]pyridin-7-yl) methanol

To a solution of methyl 5-chloro-1-isopropyl-1H-pyrrolo[3,2-b]pyridine-7-carboxylate (100 mg, 396 μmol, 1.0 eq) in anhydrous THF (10 mL) was added LiAlH₄ (30.0 mg, 791 μmol, 2.0 eq) in portions at 0° C. The reaction mixture was stirred at 0° C. for 20 min. The reaction mixture was slowly quenched with aqueous NaOH solution (1 N) (40 mL) and extracted with EtOAc (40 mL×3). The organic layer was washed with brine (40 mL×2), dried over anhydrous Na₂SO₄, filtered and concentrated under reduced pressure. The residue was purified by flash column chromatography on silica gel (PE/EA=1/1 v/v) to afford (5-chloro-1-isopropyl-1H-pyrrolo[3,2-b]pyridin-7-yl) methanol (80.0 mg, yield 90%) as a yellow solid. LC-MS (ESI): mass calcd. for C₁₁H₁₃ClN₂O, 224.07; m/z found, 225.1 [M+H]⁺.

### Step C: 5-chloro-1-isopropyl-1H-pyrrolo[3,2-b]pyridine-7-carbaldehyde

To a solution of (5-chloro-1-isopropyl-1H-pyrrolo[3,2-b]pyridin-7-yl) methanol (80.0 mg, 356 μmol, 1.0 eq) in DMSO (3.00 mL) was added IBX (Purity 45% W.t) (299 mg, 1.07 mmol, 3.0 eq) at room temperature. The reaction mixture was stirred at room temperature for 1 h. The reaction mixture was quenched with water (15 mL) and extracted with EtOAc (25 mL×3). The organic layer was washed with saturated aqueous Na₂S₂O₃ solution (30 mL×2), saturated aqueous NaHCO₃ solution (30 mL×2) and brine (20 mL), dried over anhydrous Na₂SO₄, filtered and concentrated under reduced pressure. The residue was purified by Prep-TLC(DCM/MeOH=10/1 v/v) to give 5-chloro-1-isopropyl-1H-pyrrolo[3,2-b]pyridine-7-carbaldehyde (50.0 mg, yield 63%) as a yellow solid. LC-MS (ESI): mass calcd. for C₁₁H₁₁ClN₂O, 222.06; m/z found, 223.1 [M+H]⁺.

### Intermediate 59:1-(3-((tert-butyldimethylsilyl)oxy)propyl)-5-chloro-1H-pyrrolo[3,2-b]pyridine-7-carbaldehyde

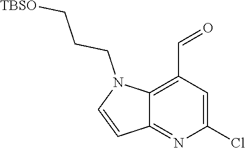 [SMILES: CC(C)(C)[Si](C)(C)OCCCn1ccc2nc(Cl)cc(C=O)c21]

### Step A: methyl 1-(3-((tert-butyldimethylsilyl)oxy)propyl)-5-chloro-1H-pyrrolo[3,2-b]pyridine-7-carboxylate

To a stirred mixture of methyl 5-chloro-1H-pyrrolo[3,2-b]pyridine-7-carboxylate (400 mg, 1.90 mmol, 1.0 eq) in DMF (20.0 mL) were added (3-bromopropoxy) (tert-butyl)dimethylsilane (962 mg, 3.80 mmol, 2.0 eq) and Cs₂CO₃ (1.86 g, 5.7 mmol, 3.0 eq). The resulting mixture was stirred at 80° C. for 16 h. After cooled to room temperature, the mixture was diluted with H₂O (40 mL) and extracted with EtOAc (50 mL×3). The combined organic layers were washed with brine (50 mL×4), dried over anhydrous Na₂SO₄, filtered and concentrated under reduced pressure. The residue was purified by flash column chromatography on silica gel (PE/EtOAc=5/1 v/v) to give methyl 1-(3-((tert-butyldimethylsilyl)oxy)propyl)-5-chloro-1H-pyrrolo[3,2-b]pyridine-7-carboxylate (400 mg, yield 55%) as a green oil. LC-MS (ESI): mass calced for: C₁₈H₂₇ClN₂O₃Si, 382.15; m/z found, 383.1 [M+H]⁺.

### Step 1 B: (1-(3-((tert-butyldimethylsilyl)oxy)propyl)-5-chloro-1H-pyrrolo[3,2-b]pyridin-7-yl) methanol

To a solution of Methyl methyl 1-(3-((tert-butyldimethylsilyl)oxy)propyl)-5-chloro-1H-pyrrolo[3,2-b]pyridine-7-carboxylate (400 mg, 1.04 mmol, 1.0 eq) in THF (20.0 mL) was added LiAlH₄ (59.5 mg, 1.57 mmol, 1.5 eq) in portions at 0° C. The resulting mixture was stirred at 0° C. for 1 h. The reaction was quenched with sodium sulfate decahydrate and filtered. The filtrate was concentrated under reduced pressure to obtain (1-(3-((tert-butyldimethylsilyl)oxy)propyl)-5-chloro-1H-pyrrolo[3,2-b]pyridin-7-yl) methanol (200 mg, yield 54%) as a brown solid. LC-MS (ESI): mass calced for: C₁₇H₂₇ClN₂O₂Si, 354.154; m/z found, 355.1 [M+H]⁺. ¹H NMR (400 MHZ, DMSO-d₆) δ 7.59 (d, J=3.2 Hz, 1H), 7.14 (s, 1H), 6.51 (d, J=3.2 Hz, 1H), 5.61 (t, J=5.8 Hz, 1H), 4.89 (d, J=5.8 Hz, 2H), 4.34 (t, J=7.2 Hz, 2H), 3.54 (t, J=5.8 Hz, 2H), 1.91-1.81 (m, 2H), 0.84 (s, 9H), 0.00 (s, 6H).

### Step C: 1-(3-((tert-butyldimethylsilyl)oxy)propyl)-5-chloro-1H-pyrrolo[3,2-b]pyridine-7-carbaldehyde

To a stirred mixture of (1-(3-((tert-butyldimethylsilyl)oxy)propyl)-5-chloro-1H-pyrrolo[3,2-b]pyridin-7-yl) methanol (200 mg, 563 μmol, 1.0 eq) in DMSO (5.0 mL) was added IBX (237 mg, 845 μmol, 1.5 eq). The resulting mixture was stirred at 25° C. for 2 h. The reaction mixture was quenched with saturated aqueous Na₂S₂O₃ solution (50 mL) and extracted with EtOAc (50 mL×3). The combined organic layers were washed with brine (50 mL), dried over anhydrous Na₂SO₄ and concentrated under reduced pressure. The residue was purified by flash column chromatography on silica gel (PE/EtOAc=3/1 v/v) to give 1-(3-((tert-butyldimethylsilyl)oxy)propyl)-5-chloro-1H-pyrrolo[3,2-b]pyridine-7-carbaldehyde (100 mg, yield 50%) as a yellow solid. LC-MS (ESI): mass calced for: C₁₇H₂₅ClN₂O₂Si, 352.14; m/z found, 353.1 [M+H]⁺.

### Intermediate 60:5-chloro-1-cyclopropyl-1H-pyrrolo[3,2-b]pyridine-7-carbaldehyde

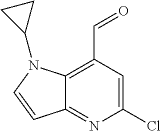 [SMILES: O=Cc1cc(Cl)nc2ccn(C3CC3)c12]

### Step A: methyl 5-chloro-1-cyclopropyl-1H-pyrrolo[3,2-b]pyridine-7-carboxylate

To a solution of cyclopropylboronic acid (367 mg, 4.3 mmol, 3.0 eq) and methyl 5-chloro-1H-pyrrolo[3,2-b]pyridine-7-carboxylate (300 mg, 1.4 mmol, 1.0 eq) in DCE (8.0 mL) were added 2,2′-Bipyridine (445 mg, 2.9 mmol, 2.0 eq), Na₂CO₃ (755 mg, 7.1 mmol, 5.0 eq), and Cu (OAc)₂ (517 mg, 2.9 mmol, 2.0 eq) at 25° C. The reaction mixture was stirred under O₂ (1 atm) at 70° C. for 4 h. After cooled to room temperature, the reaction mixture was filtered and the organic layer was quenched with water (20 mL) and extracted with DCM (30 mL×3). The organic layer was washed with brine (30 mL), dried over Na₂SO₄ and concentrated under reduced pressure. The residue was purified by flash column chromatography on silica gel (PE/EA=2/1 v/v) to give methyl 5-chloro-1-cyclopropyl-1H-pyrrolo[3,2-b]pyridine-7-carboxylate (180 mg, yield 50%) as a yellow solid. LC-MS (ESI): mass calcd. for C₁₂H₁₁ClN₂O₂, 250.1; m/z found, 251.1 [M+H]⁺.

### Step B: (5-chloro-1-cyclopropyl-1H-pyrrolo[3,2-b]pyridin-7-yl) methanol

To a solution of methyl 5-chloro-1-cyclopropyl-1H-pyrrolo[3,2-b]pyridine-7-carboxylate (300 mg, 1.2 mmol, 1.0 eq) in THF (5.0 mL) was added LiAlH₄ (90.8 mg, 2.4 mmol, 2.0 eq) at 0° C. and the reaction mixture was stirred under N₂ at 0° C. for 5 min. The reaction mixture was quenched with water (20 mL) and extracted with EA (20 mL×3). The organic layer was washed with brine (30 mL), dried over Na₂SO₄ and concentrated under reduced pressure. The residue was purified by flash column chromatography on silica gel (PE/EA=2/1 v/v) to give (5-chloro-1-cyclopropyl-1H-pyrrolo[3,2-b]pyridin-7-yl) methanol (250 mg, yield 94%) as a yellow solid. LC-MS (ESI): mass calcd. for C₁₁H₁₁ClN₂O, 222.1; m/z found, 223.1 [M+H]⁺.

### Step C: 5-chloro-1-cyclopropyl-1H-pyrrolo[3,2-b]pyridine-7-carbaldehyde

To a solution of (5-chloro-1-cyclopropyl-1H-pyrrolo[3,2-b]pyridin-7-yl) methanol (250 mg, 1.1 mmol, 1.0 eq) in DMSO (5.0 mL) was added IBX (Purity 45% W.t) (943 mg, 3.4 mmol, 3.0 eq) at room temperature and the reaction mixture was stirred at room temperature for 20 min. The reaction mixture was quenched with water (20 mL) and extracted with EA (30 mL×3). The organic layer was washed with brine (30 mL×3), dried over Na₂SO₄, filtered and concentrated under reduced pressure. The residue was purified by flash column chromatography on silica gel (PE/EA=10/1 v/v) to give 5-chloro-1-cyclopropyl-1H-pyrrolo[3,2-b]pyridine-7-carbaldehyde (230 mg, yield 93%) as a yellow solid. LC-MS (ESI): mass calcd. for C₁₁H₉ClN₂O, 220.0; m/z found, 221.0 [M+H]⁺.

### Intermediate 61:1-(2-((tert-butyldimethylsilyl)oxy)ethyl)-5-chloro-1H-pyrrolo[3,2-b]pyridine-7-carbaldehyde

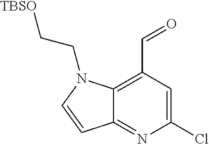 [SMILES: CC(C)(C)[Si](C)(C)OCCn1ccc2nc(Cl)cc(C=O)c21]

### Step A: methyl 1-(2-((tert-butyldimethylsilyl)oxy)ethyl)-5-chloro-1H-pyrrolo[3,2-b]pyridine-7-carboxylate

To a stirred mixture of methyl 5-chloro-1H-pyrrolo[3,2-b]pyridine-7-carboxylate (500 mg, 2.37 mmol, 1.0 eq) in DMF (20.0 mL) were added (2-bromoethoxy) (tert-butyl)dimethylsilane (1.14 g, 4.75 mmol, 2.0 eq) and Cs₂CO₃ (2.32 g, 7.12 mmol, 3.0 eq). The resulting mixture was stirred at 80° C. for 16 h. After cooled to room temperature, the mixture was diluted with H₂O (40 mL) and extracted with EtOAc (50 mL×3). The combined organic layers were washed with brine (50 mL×4), dried over anhydrous Na₂SO₄, filtered and concentrated under reduced pressure. The residue was purified by flash column chromatography on silica gel (PE/EtOAc=5/1 v/v) to give methyl 1-(2-((tert-butyldimethylsilyl)oxy)ethyl)-5-chloro-1H-pyrrolo[3,2-b]pyridine-7-carboxylate (800 mg, yield 91%) as a green oil. LC-MS (ESI): mass calced for: C₁₇H₂₅ClN₂O₃Si, 368.13; m/z found, 369.1 [M+H]⁺.

### Step B: (1-(2-((tert-butyldimethylsilyl)oxy)ethyl)-5-chloro-1H-pyrrolo[3,2-b]pyridin-7-yl) methanol

To a solution of Methyl 1-(2-((tert-butyldimethylsilyl)oxy)ethyl)-5-chloro-1H-pyrrolo[3,2-b]pyridine-7-carboxylate (600 mg, 1.63 mmol, 1.0 eq) in THF (20.0 mL) was added LiAlH₄ (92.6 mg, 2.44 mmol, 1.5 eq) in portions at 0° C. The resulting mixture was stirred at 0° C. for 1 h. The reaction was quenched with sodium sulfate decahydrate and filtered. The filtrate was concentrated under reduced pressure to obtain (1-(2-((tert-butyldimethylsilyl)oxy)ethyl)-5-chloro-1H-pyrrolo[3,2-b]pyridin-7-yl) methanol (500 mg, yield 90%) as a brown solid. LC-MS (ESI): mass calced for: C₁₆H₂₅ClN₂O₂Si, 340.14; m/z found, 341.1 [M+H]⁺.

### Step C: 1-(2-((tert-butyldimethylsilyl)oxy)ethyl)-5-chloro-1H-pyrrolo[3,2-b]pyridine-7-carbaldehyde

To a stirred mixture of (1-(2-((tert-butyldimethylsilyl)oxy)ethyl)-5-chloro-1H-pyrrolo[3,2-b]pyridin-7-yl) methanol (500 mg, 1.47 mmol, 1.0 eq) in DMSO (10.0 mL) was added IBX (616 mg, 2.20 mmol, 1.5 eq). The resulting mixture was stirred at 25° C. for 2 h. The reaction mixture was quenched with saturated aqueous Na₂S₂O₃ solution (50 mL) and extracted with EtOAc (50 mL×3). The combined organic layers were washed with brine (50 mL), dried over anhydrous Na₂SO₄ and concentrated under reduced pressure. The residue was purified by flash column chromatography on silica gel (PE/EtOAc=3/1 v/v) to give 1-(2-((tert-butyldimethylsilyl)oxy)ethyl)-5-chloro-1H-pyrrolo[3,2-b]pyridine-7-carbaldehyde (350 mg, yield 70%) as a yellow solid. LC-MS (ESI): mass calced for: C₁₆H₂₃ClN₂O₂Si, 338.12; m/z found, 339.1 [M+H]⁺.

### Intermediate 62:3-amino-5-chloro-1-isopropyl-1H-pyrazolo[4,3-b]pyridine-7-carbaldehyde

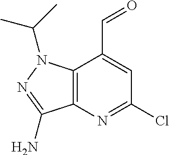 [SMILES: CC(C)n1nc(N)c2nc(Cl)cc(C=O)c21]

### Step A: 7-bromo-5-chloro-1-isopropyl-3-nitro-1H-pyrazolo[4,3-b]pyridine

To a solution of 7-bromo-5-chloro-3-nitro-1H-pyrazolo[4,3-b]pyridine (2.00 g, 7.21 mmol, 1.0 eq) in DMF (20.0 mL) was added Cs₂CO₃ (4.70 g, 14.4 mmol, 2.0 eq) at 0° C. . . . After 30 min, 2-iodopropane (1.84 g, 1.08 mL, 10.8 mmol, 1.5 eq) was added to above mixture and the mixture was stirred 100° C. for 2.0 h. After cooled to room temperature, the reaction mixture was quenched with saturated aqueous NH₄Cl solution (50 mL) and extracted with EA (50 mL×4). The organic layer was washed with brine (50 mL×4), dried over anhydrous Na₂SO₄, filtered and concentrated in vacuum. The crude product was purified by flash column chromatography on silica gel (PE/EA=2/1 v/v) to give 7-bromo-5-chloro-1-isopropyl-3-nitro-1H-pyrazolo[4,3-b]pyridine (1.00 g, yield 44%) as a yellow oil. LC-MS (ESI): mass calced for: C₉H₈BrClN₄O₂Si, 317.95; m/z found, 318.9 [M+H]⁺.

### Step B: 7-bromo-5-chloro-1-isopropyl-1H-pyrazolo[4,3-b]pyridin-3-amine

To a mixture of 7-bromo-5-chloro-1-isopropyl-3-nitro-1H-pyrazolo[4,3-b]pyridine (1.00 g, 3.13 mmol, 1.0 eq) in water (5 mL), ethanol (10 mL), and THF (10.0 mL) were added iron (874 mg, 15.6 mmol, 5.0 eq) and ammonium chloride (837 mg, 15.6 mmol, 5.0 eq). The mixture was stirred under 80° C. for 1 h. After cooled to room temperature, the mixture was filtered and the filtrate was concentrated in vacuum. The residue was diluted with EA (100 mL), washed with brine (50 mL), dried over anhydrous sodium sulfate, filtered and concentrated under reduced pressure. The residue was purified by flash column chromatography on silica gel (PE/EA=9/1 v/v) to give 7-bromo-5-chloro-1-isopropyl-1H-pyrazolo[4,3-b]pyridin-3-amine (680 mg, yield 75%) as a yellow solid. LC-MS (ESI): mass calced for: C₉H₁₀BrClN₄Si, 287.98; m/z found, 289.1 [M+H]⁺. ¹H NMR (400 MHZ, DMSO-d6) δ 7.74 (s, 1H), 5.77 (s, 2H), 5.46-5.36 (m, 1H), 1.40 (d, J=6.4 Hz, 6H).

### Step C: 5-chloro-1-isopropyl-7-vinyl-1H-pyrazolo[4,3-b]pyridin-3-amine

To a stirred mixture of 7-bromo-5-chloro-1-isopropyl-1H-pyrazolo[4,3-b]pyridin-3-amine (650 mg, 2.24 mmol, 1.0 eq) in 1,4-Dioxane (10.0 mL) and H₂O (1.50 mL) were added Potassium phosphate tribasic (1.43 g, 6.73 mmol, 3.0 eq), Potassium vinyltrifluoroborate (451 mg, 3.37 mmol, 1.5 eq), and 1,1′-Bis(diphenylphosphino) ferrocene-palladium (II) dichloride (329 mg, 449 μmol, 0.2 eq). The resulting mixture was stirred under N₂ at 80° C. for 2 h. After cooled to room temperature, the reaction mixture was diluted with water (20 mL) and extracted with EtOAc (30 mL×3). The combined organic layers was washed with brine (50 mL), dried over anhydrous Na₂SO₄, filtered and concentrated in vacuum. The residue was purified by flash column chromatography on silica gel (PE/EtOAc=3/1 v/v) to give 5-chloro-1-isopropyl-7-vinyl-1H-pyrazolo[4,3-b]pyridin-3-amine (310 mg, yield 58%) as yellow oil. LC-MS (ESI): mass calced for: C₁₁H₁₃ClN₄, 236.08; m/z found, 273.2 [M+H]⁺.

### Step D: 3-amino-5-chloro-1-isopropyl-1H-pyrazolo[4,3-b]pyridine-7-carbaldehyde

To a stirred mixture of 5-chloro-1-isopropyl-7-vinyl-1H-pyrazolo[4,3-b]pyridin-3-amine (110 mg, 465 μmol, 1.0 eq) in acetone (10 mL) and H₂O (10 mL) were added with potassium osmate (VI) dihydrate (85.6 mg, 232 μmol, 0.5 eq) and NMO (136 mg, 1.16 mmol, 2.5 eq). The resulting mixture was stirred at room temperature for 2 h. Sodium metaperiodate (497 mg, 123 μL, 2.32 mmol, 5.0 eq) was added to above mixture and the resulting mixture was stirred at room temperature for 2 h. The reaction mixture was diluted with H₂O (50 mL) and extracted with EtOAc (50 mL×3). The combined organic layers were washed with brine (50 mL×3), dried over anhydrous Na₂SO₄ and concentrated. The residue was purified by flash column chromatography on silica gel (PE/EtOAc=1/1 v/v) to give 3-amino-5-chloro-1-isopropyl-1H-pyrazolo[4,3-b]pyridine-7-carbaldehyde (35.0 mg, yield 32%) as a white solid. LC-MS (ESI): mass calced for: C₁₀HuClN40, 238.06; m/z found, 271.1 [M+H]⁺

### Intermediate 63:3-amino-5-chloro-1-ethyl-1H-pyrazolo[4,3-b]pyridine-7-carbaldehyde

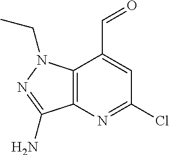

### Step A: 7-bromo-5-chloro-1-ethyl-3-nitro-1H-pyrazolo[4,3-b]pyridine

To a solution of 7-bromo-5-chloro-3-nitro-1H-pyrazolo[4,3-b]pyridine (2.00 g, 7.21 mmol, 1.0 eq) in DMF (20 mL) was added Cs₂CO₃ (4.70 g, 14.4 mmol, 2.0 eq) at room temperature and the mixture was stirred for 10 min. Ethyl iodide (1.12 g, 578 μL, 7.21 mmol, 1.0 eq) was added to above mixture and the mixture was stirred under N₂ at 60° C. for 4 h. After cooled to room temperature, the reaction mixture was diluted with EA (60 mL) and filtered. The filtrate was washed with brine (30 mL×4), dried over anhydrous Na₂SO₄, filtered and concentrated under reduced pressure. The residue was purified by flash column chromatography on silica gel (ethyl acetate in petroleum ether, from 0 to 30%) to give 7-bromo-5-chloro-1-ethyl-3-nitro-1H-pyrazolo[4,3-b]pyridine (940 mg, yield 42%) as a yellow solid. LC-MS (ESI): mass calcd. for C₈H₆BrClN₄O₂, 305.52; m/z found, 305.2 [M+H+1]*. ¹H NMR (400 MHZ, DMSO-d₆) δ 8.22 (s, 1H), 4.87 (q, J=7.2 Hz, 2H), 1.53 (t, J=7.2 Hz, 3H).

### Step B: 7-bromo-5-chloro-1-ethyl-1H-pyrazolo[4,3-b]pyridin-3-amine

To a solution of 7-bromo-5-chloro-1-ethyl-3-nitro-1H-pyrazolo[4,3-b]pyridine (940 mg, 3.08 mmol, 1.0 eq) and ammonium chloride (823 mg, 15.4 mmol, 5.0 eq) in THF (10.0 mL), EtOH (10.0 mL), and Water (5.00 mL) was added iron (859 mg, 15.4 mmol, 5.0 eq). The reaction mixture was stirred at 70° C. for 1 h. After cooled to room temperature, the mixture was filtered and the cake was washed with EA (30 mL×3). The filtrate was concentrated under reduced pressure and the residue was diluted with EA (40 mL). The organic layer was washed with water (50 mL) and brine (50 mL), dried over anhydrous Na₂SO₄, filtered and concentrated under reduced pressure. The crude product was purified by flash column chromatography on silica gel (ethyl acetate in petroleum ether, from 0% to 30%) to give 7-bromo-5-chloro-1-ethyl-1H-pyrazolo[4,3-b]pyridin-3-amine (720 mg, yield 84%) as a yellow solid. LC-MS (ESI): mass calcd. for C₈H₈BrClN₄, 275.53; m/z found, 275.4 [M+H]⁺. ¹H NMR (400 MHZ, DMSO-d₆) δ 7.74 (s, 1H), 5.75 (s, 2H), 4.44 (q, J=7.2 Hz, 2H), 1.29 (t, J=7.2 Hz, 3H).

### Step C: 5-chloro-1-ethyl-7-vinyl-1H-pyrazolo[4,3-b]pyridin-3-amine

To a solution of 7-bromo-5-chloro-1-ethyl-1H-pyrazolo[4,3-b]pyridin-3-amine (720 mg, 2.61 mmol, 1.0 eq) and K₂CO₃ (722 mg, 5.23 mmol, 2.0 eq) in 1,4-Dioxane (10.0 mL) and Water (1.00 mL) were added Potassium vinyltrifluoroborate (525 mg, 3.92 mmol, 15 eq) and Pd(dppf)Cl₂ (191 mg, 261 μmol, 0.1 eq). The reaction mixture was stirred under N₂ at 90° C. for 5 h. After cooled to room temperature, the mixture was filtered and the cake was washed with EtOAc (30 mL×3). The filtrate was concentrated and the residue was diluted with EtOAc (40 mL). The organic layer was washed with water (50 mL) and brine (50 mL), dried over anhydrous Na₂SO₄, filtered and concentrated under reduced pressure. The crude product was purified by flash column chromatography on silica gel (ethyl acetate in petroleum ether, from 0% to 30%) give 5-chloro-1-ethyl-7-vinyl-1H-pyrazolo[4,3-b]pyridin-3-amine (330 mg, yield 56%) as a yellow solid. LC-MS (ESI): mass calcd. for C₁₀H₁₁ClN₄, 222.68; m/z found, 223.1 [M+H]⁺.

### Step D: 3-amino-5-chloro-1-ethyl-1H-pyrazolo[4,3-b]pyridine-7-carbaldehyde

To a solution of 5-chloro-1-ethyl-7-vinyl-1H-pyrazolo[4,3-b]pyridin-3-amine (300 mg, 1.35 mmol, 1.0 eq) and N-methylmorpholine N-oxide (158 mg, 1.35 mmol, 1.0 eq) in acetone (10.0 mL) and water (5.00 mL) was added with potassium osmate (VI) dihydrate (49.6 mg, 135 μmol, 0.1 eq). The reaction mixture was stirred at 25° C. for 1 h. Then sodium metaperiodate (864 mg, 214 μL, 4.04 mmol, 3.0 eq) was added to above mixture and the mixture was stirred at 25° C. for 2 h. The mixture was filtered and the cake was washed with EtOAc (30 mL×3). The filtrate was concentrated and the residue was diluted with EtOAc (40 mL). The organic layer was washed with water (50 mL) and brine (50 mL), dried over anhydrous Na₂SO₄, filtered and concentrated under reduced pressure. The crude product was purified by flash column chromatography on silica gel (ethyl acetate in petroleum ether, from 0% to 30%) to give 3-amino-5-chloro-1-ethyl-1H-pyrazolo[4,3-b]pyridine-7-carbaldehyde (260 mg, yield 85%) as a yellow solid. LC-MS (ESI): mass calcd. for C₉H₉ClN₄O, 224.65; m/z found, 225.0 [M+H]⁺.

### Intermediate 64:5-chloro-3-(dimethylamino)-1-((2-(trimethylsilyl)ethoxy)methyl)-1H-pyrazolo[4,3-b]pyridine-7-carbaldehyde

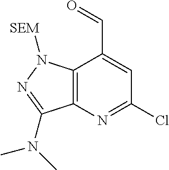 [SMILES: CN(C)c1nn(COCC[Si](C)(C)C)c2c(C=O)cc(Cl)nc12]

### Step A: 5-chloro-N,N-dimethyl-1-((2-(trimethylsilyl)ethoxy)methyl)-7-vinyl-1H-pyrazolo[4,3-b]pyridin-3-amine

To a solution of 7-bromo-5-chloro-N,N-dimethyl-1-((2-(trimethylsilyl)ethoxy)methyl)-1H-pyrazolo[4,3-b]pyridin-3-amine (300 mg, 739 μmol, 1.0 eq) and potassium vinyltrifluoroborate (109 mg, 813 μmol, 1.1 eq) in dioxane (5 mL) and water (0.5 mL) were added potassium phosphate (471 mg, 2.22 mmol, 3.0 eq) and PdCl₂ (dppf) (54.1 mg, 73.9 μmol, 0.1 eq). The mixture was stirred under N₂ at 80° C. for 16 h. The reaction mixture was cooled to room temperature and concentrated under reduced pressure. The residue was poured into water (5 mL) and extracted with EtOAc (10 mL×3). The organic layer was dried over MgSO₄, filtered and concentrated under reduced pressure. The residue was purified by flash column chromatography on silica gel (PE/EA=20/1 v/v) to obtain 5-chloro-N,N-dimethyl-1-((2-(trimethylsilyl)ethoxy)methyl)-7-vinyl-1H-pyrazolo[4,3-b]pyridin-3-amine (200 mg, yield 69%) as a yellow oil. LC-MS (ESI): mass calced for: C₁₆H₂₅ClN₄OSi, 352.1; m/z found, 353.1 [M+H]⁺.

### Step B: 5-chloro-3-(dimethylamino)-1-((2-(trimethylsilyl)ethoxy)methyl)-1H-pyrazolo[4,3-b]pyridine-7-carbaldehyde

A solution of 5-chloro-N,N-dimethyl-1-((2-(trimethylsilyl)ethoxy)methyl)-7-vinyl-1H-pyrazolo[4,3-b]pyridin-3-amine (170 mg, 482 μmol, 1.0 eq), 4-methylmorpholine 4-oxide (141 mg, 1.20 mmol, 2.5 eq), and potassium osmate (VI) dihydrate (32.0 mg, 96.3 μmol, 0.2 eq) in acetone (5.00 mL) and H₂O (2.50 mL) was stirred at room temperature for 0.5 h. Then sodium periodate (515 mg, 2.41 mmol, 5.0 eq) was added to above mixture and the mixture was stirred at room temperature for 2 h. After filtration, the filtrate was diluted with water (6 mL) and extracted with EA (10 mL×3). The organic layer was dried over anhydrous Na₂SO₄, filtered and concentrated under reduced pressure. The crude product was purified by=Prep-TLC(PE/EA 20/1 v/v) to give 5-chloro-3-(dimethylamino)-1-((2-(trimethylsilyl)ethoxy)methyl)-1H-pyrazolo[4,3-b]pyridine-7-carbaldehyde (100 mg, yield 53%) as a yellow oil. LC-MS (ESI): mass calced for: C₁₅H₂₃ClN₄O₂Si 354.1; m/z found, 355.1 [M+H]⁺.

### Intermediate 65:7-(bromomethyl)-5-chloro-3-methyl-3H-imidazo[4,5-b]pyridine

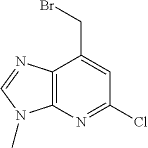

### Step A: 6-chloro-N,4-dimethyl-3-nitropyridin-2-amine

To a solution of 2,6-dichloro-4-methyl-3-nitropyridine (1.00 g, 4.83 mmol, 1.0 eq) in THF (20.0 mL) was added DIPEA (1.87 g, 14.5 mmol, 3.0 eq). Then methyl amine (2.0 M in THF) (165 mg, 2.66 mL, 5.31 mmol, 1.1 eq) was added to above mixture at −30° C. and the mixture was stirred at −30° C. for 1 h. After evaporation, the residue was purified by flash column chromatography on silica gel (ethyl acetate in petroleum ether, 8% v/v) to obtain 6-chloro-N,4-dimethyl-3-nitropyridin-2-amine (950 mg, yield 97%) as a yellow solid. LC-MS (ESI): mass calced for: C₇H₈ClN₃O₂, 201.61; m/z found, 202.2 [M+H]⁺. ¹H NMR (400 MHZ, DMSO-d₆) δ 7.94 (q, J=4.6 Hz, 1H), 6.71 (s, 1H), 2.89 (d, J=4.6 Hz, 3H), 2.38 (s, 3H).

### Step B: 6-chloro-N₂,4-dimethylpyridine-2,3-diamine

To a solution of 6-chloro-N,4-dimethyl-3-nitropyridin-2-amine (950 mg, 4.71 mmol, 1.0 eq) in EtOH (20.0 mL) and AcOH (2.00 mL) was added Iron (2.63 g, 47.1 mmol, 10.0 eq). The mixture was stirred at 30° C. for 30 min. The reaction mixture is filtered and the filtrate was concentrated under reduced pressure. The residue is diluted with EA (50 mL), washed with saturated aqueous NaHCO₃ solution (30 mL) and brine (30 mL), dried over MgSO₄, filtered and concentrated under reduced pressure. The crude product was purified by flash column chromatography on silica gel (ethyl acetate in petroleum ether, 30% v/v) to obtain 6-chloro-N₂,4-dimethylpyridine-2,3-diamine (330 mg, yield 40%) as a brown oil. LC-MS (ESI): mass calced for: C₇H₁₀ClN₃, 171.63; m/z found, 172.1 [M+H]⁺

### Step C: 5-chloro-3,7-dimethyl-3H-imidazo[4,5-b]pyridine

To a solution of 6-chloro-N₂,4-dimethylpyridine-2,3-diamine (330 mg, 1.92 mmol, 1.0 eq) in trimethoxymethane (20.0 mL) was added FA (0.40 mL). The mixture was stirred at 90° C. for 2 h. The reaction mixture was cooled to room temperature and concentrated in vacuum. The crude product was purified by Prep-TLC(PE/EA=2/1 v/v) to obtain 5-chloro-3,7-dimethyl-3H-imidazo[4,5-b]pyridine (330 mg, yield 94%) as a white solid. LC-MS (ESI): mass calced for: C₈H&ClN₃, 181.62; m/z found, 182.1 [M+H]⁺. ¹H NMR (400 MHZ, DMSO-d6) § 8.38 (s, 1H), 7.21 (s, 1H), 3.80 (s, 3H), 2.56 (s, 3H).

### Step D: 7-(bromomethyl)-5-chloro-3-methyl-3H-imidazo[4,5-b]pyridine

To a solution of 5-chloro-3,7-dimethyl-3H-imidazo[4,5-b]pyridine (330 mg, 1.82 mmol, 1.0 eq) in CCl₄ (15.0 mL) were added NBS (356 mg, 2.00 mmol, 1.1 eq) and Benzoyl peroxide (44.0 mg, 182 μmol, 0.1 eq). The mixture was stirred at 90° C. for 16 h. After cooled to room temperature, the reaction mixture was quenched with water (20 mL) and extracted with DCM (20 mL×3). The organic layer was dried over MgSO₄, filtered and concentrated under reduced pressure. The crude product was purified by Prep-TLC(PE/EA=4/1 v/v) to obtain 7(bromomethyl)-5-chloro-3-methyl-3H-imidazo[4,5-b]pyridine (210 mg, yield 44%) as a white solid. LC-MS (ESI): mass calced for: C₈HBrClN₃, 260.52; m/z found, 261.2 [M+H]⁺.

### Intermediate 66:5-chloro-3-(methylamino)-1-((2-(trimethylsilyl)ethoxy)methyl)-1H-pyrazolo[4,3-b]pyridine-7-carbaldehyde

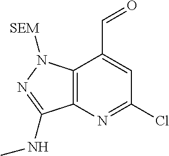 [SMILES: CNc1nn(COCC[Si](C)(C)C)c2c(C=O)cc(Cl)nc12]

### Step A: 7-bromo-5-chloro-N-methyl-1-((2-(trimethylsilyl)ethoxy)methyl)-1H-pyrazolo[4,3-b]pyridin-3-amine

To a solution of 7-bromo-5-chloro-1-((2-(trimethylsilyl)ethoxy)methyl)-1H-pyrazolo[4,3-b]pyridin-3-amine (2.00 g, 5.29 mmol, 1.0 eq) and Cs₂CO₃ (8.63 g, 26.5 mmol, 5.0 eq) in DMF (20.0 mL) was added MeI (7.52 g, 3.31 mL, 52.9 mmol, 10.0 eq) at room temperature. The reaction mixture was stirred at 100° C. for 16 h. After cooled to room temperature, the reaction mixture was quenched with water (50 mL) and extracted with EtOAc (50 mL×3). The organic layer was washed with brine (50 mL×4), dried over anhydrous Na₂SO₄, filtered and concentrated under reduced pressure. The residue was purified by flash (ethyl acetate in petroleum ether, 15% v/v) to give 7-bromo-5-chloro-N-methyl-1-((2-(trimethylsilyl)ethoxy)methyl)-1H-pyrazolo[4,3-b]pyridin-3-amine (410 mg, yield 20%) as a white solid. LC-MS (ESI): mass calced for: C₁₃H₂OBrClN₄OSi, 390.03; m/z found, 391.3 [M+H]⁺. ¹H NMR (400 MHZ, DMSO-d₆) δ 7.97 (s, 1H), 6.57 (q, J=5.0 Hz, 1H), 5.82 (s, 2H), 3.66 (t, J=8.0 Hz, 2H), 2.97 (d, J=5.0 Hz, 3H), 0.89 (t, J=8.0 Hz, 2H), 0.00 (s, 9H).

### Step B: 5-chloro-N-methyl-1-((2-(trimethylsilyl)ethoxy)methyl)-7-vinyl-1H-pyrazolo[4,3-b]pyridin-3-amine

To a stirred mixture of 7-bromo-5-chloro-N-methyl-1-((2-(trimethylsilyl)ethoxy)methyl)-1H-pyrazolo[4,3-b]pyridin-3-amine (810 mg, 2.07 mmol, 1.0 eq) in 1,4-Dioxane (10.0 mL) and H₂O (1.50 mL) were added Potassium phosphate tribasic (658 mg, 3.10 mmol, 1.5 eq), Potassium vinyl trifluoroborate (55.4 mg, 414 μmol, 0.2 eq), and 1,1′-Bis(diphenylphosphino) ferrocene-palladium (II) dichloride (151 mg, 207 μmol, 0.1 eq). The resulting mixture was stirred under Neat 80° C. for 2 h. After cooled to room temperature, the reaction mixture was diluted with water (20 mL) and extracted with EtOAc (30 mL×3). The combined organic layers was washed with brine (20 mL), dried over anhydrous Na₂SO₄, filtered and concentrated in vacuum. The residue was purified by flash column chromatography on silica gel (PE/EtOAc=3/1 v/v) to give 5-chloro-N-methyl-1-((2-(trimethylsilyl)ethoxy)methyl)-7-vinyl-1H-pyrazolo[4,3-b]pyridin-3-amine (340 mg, yield 48%) as yellow oil. LC-MS (ESI): mass calced for: C₁₅H₂₃ClN₄OSi, 338.13; m/z found, 339.1 [M+H]⁺. ¹H NMR (400 MHZ, DMSO-d₆) δ 7.66 (s, 1H), 7.36 (dd, J=17.4, 11.0 Hz, 1H), 6.39 (d, J=5.0 Hz, 1H), 6.31 (d, J=17.2 Hz, 1H), 5.82 (d, J=7.8 Hz, 1H), 5.64 (s, 2H), 3.65-3.60 (m, 2H), 2.95 (d, J=5.0 Hz, 3H), 0.90 (t, J=8.0 Hz, 2H), 0.00 (s, 9H).

### Step C: 5-chloro-3-(methylamino)-1-((2-(trimethylsilyl)ethoxy)methyl)-1H-pyrazolo[4,3-b]pyridine-7-carbaldehyde

To a stirred mixture of 5-chloro-N-methyl-1-((2-(trimethylsilyl)ethoxy)methyl)-7-vinyl-1H-pyrazolo[4,3-b]pyridin-3-amine (120 mg, 354 μmol, 1.0 eq) in acetone (10 mL) and H₂O (10 mL) were added with potassium osmate (VI) dihydrate (39.1 mg, 106 μmol, 0.3 eq) and NMO (83.0 mg, 708 μmol, 2.0 eq). The resulting mixture was stirred at room temperature for 2 h. Sodium metaperiodate (454 mg, 112 μL, 2.12 mmol, 6.0 eq) was added to above mixture and the resulting mixture was stirred at room temperature for 2 h. The reaction mixture was diluted with H₂O (50 mL) and extracted with EtOAc (50 mL×3). The combined organic layers were washed with brine (50 mL×3), dried over anhydrous Na₂SO₄, filtered and concentrated in vacuum. The residue was purified by flash column chromatography on silica gel (PE/EtOAc=1/1 v/v) to give 5-chloro-3-(methylamino)-1-((2-(trimethylsilyl)ethoxy)methyl)-1H-pyrazolo[4,3-b]pyridine-7-carbaldehyde (50.0 mg, yield 41%) as a white solid. LC-MS (ESI): mass calced for: C₁₄H₂₁ClN₄O₂Si, 340.1; m/z found, 341.0 [M+H]⁺.

### Intermediate 67:3-amino-5-chloro-1-methyl-1H-pyrazolo[4,3-b]pyridine-7-carbaldehyde

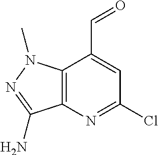

### Step A: 7-bromo-5-chloro-1-methyl-3-nitro-1H-pyrazolo[4,3-b]pyridine

To a solution of 7-bromo-5-chloro-3-nitro-1H-pyrazolo[4,3-b]pyridine (1.50 g, 5.41 mmol, 1.0 eq) in DMF (20 mL) was added Cs₂CO₃ (4.40 g, 13.5 mmol, 2.5 eq) at room temperature and the mixture was stirred for 10 min. MeI (1.15 g, 507 μL, 8.11 mmol, 1.5 eq) was added to above mixture and the mixture was stirred under N₂ at room temperature for 2 h. The reaction mixture was diluted with EA (150 mL) and filtered. The filtrate was washed with brine (60 mL×5), dried over anhydrous Na₂SO₄, filtered and concentrated under reduced pressure. The residue was purified by flash column chromatography on silica gel (EA/PE=1/5 to 1/2 v/v) to give 7-bromo-5-chloro-1-methyl-3-nitro-1H-pyrazolo[4,3-b]pyridine (1.17 g, 4.01 mmol, yield 74%) as a yellow solid. LC-MS (ESI): mass calced for: C₇H₄BrClN₄O₂, 289.92; m/z found, 290.93 [M+H]⁺.

### Step B: 7-bromo-5-chloro-1-methyl-1H-pyrazolo[4,3-b]pyridin-3-amine

To a solution of 7-bromo-5-chloro-1-methyl-3-nitro-1H-pyrazolo[4,3-b]pyridine (1.17 g, 4.01 mmol, 1.0 eq) and Ammonium chloride (1.07 g, 20.1 mmol, 5.0 eq) in THF (40 mL), EtOH (40 mL), and H₂O (20 mL) was added Iron (1.12 g, 20.1 mmol, 5.0 eq) at room temperature. The mixture was stirred at 70° C. for 1 h. After cooled to room temperature, the mixture was filtered and the cake was washed with EA (50 mL×3). The organic layer was concentrated and the residue was diluted with EA (150 mL). The organic layer was washed with water (50 mL) and brine (50 mL), dried over anhydrous sodium sulfate, filtered and concentrated under reduced pressure to give 7-bromo-5-chloro-1-methyl-1H-pyrazolo[4,3-b]pyridin-3-amine (1.03 g, yield 98%) as a yellow solid. LC-MS (ESI): mass calced for: C₇H₆BrClN₄, 259.95; m/z found, 260.95 [M+H]⁺.

¹H NMR (400 MHZ, DMSO-d₆) δ 7.84 (s, 1H), 5.83 (s, 2H), 4.13 (s, 3H).

### Step C: 5-chloro-1-methyl-7-vinyl-1H-pyrazolo[4,3-b]pyridin-3-amine

To a solution of 7-bromo-5-chloro-1-methyl-1H-pyrazolo[4,3-b]pyridin-3-amine (650 mg, 2.49 mmol, 1.0 eq), Potassium vinyltrifluoroborate (433 mg, 3.23 mmol, 1.3 eq), and Potassium phosphate tribasic (1.58 g, 7.46 mmol, 3.0 eq) in 1,4-Dioxane (25.0 mL) and Water (6.00 mL) was added 1,1′-Bis(diphenylphosphino) ferrocene-palladium (II) dichloride (182 mg, 249 μmol, 0.1 eq). The reaction mixture was stirred under nitrogen at 80° C. for 2 h. After cooled to room temperature, the mixture was filtered and the filtrate was concentrated in vacuum. The crude product was purified by flash column chromatography on silica gel (PE/EA=3/1 v/v) to provide 5-chloro-1-methyl-7-vinyl-1H-pyrazolo[4,3-b]pyridin-3-amine (348 mg, yield 67%) as a yellow solid. LC-MS (ESI): mass calced for: C₉H₉ClN₄, 208.05; m/z found, 209.1 [M+H]⁺. ¹H NMR (400 MHZ, DMSO-d₆) δ 7.40 (s, 1H), 7.39-7.32 (m, 1H), 6.11 (d, J=17.2 Hz, 1H), 5.70 (d, J=11.0 Hz, 1H), 5.53 (s, 2H), 3.92 (s, 3H).

### Step D: 1-(3-amino-5-chloro-1-methyl-1H-pyrazolo[4,3-b]pyridin-7-yl) ethane-1,2-diol

To a solution of 5-chloro-1-methyl-7-vinyl-1H-pyrazolo[4,3-b]pyridin-3-amine (100 mg, 479 μmol, 1.0 eq) and potassium osmate (VI) dihydrate (17.7 mg, 47.9 μmol, 0.1 eq) in Acetone (6.00 mL) and Water (3.00 mL) was added with potassium osmate (VI) dihydrate (17.7 mg, 47.9 μmol, 0.1 eq). The reaction mixture was stirred at room temperature for 2 h. The mixture was filtered and the filtrate was concentrated in vacuum. The crude product was purified by Prep-TLC(DCM/MeOH=10/1 v/v) to give 1-(3-amino-5-chloro-1-methyl-1H-pyrazolo[4,3-b]pyridin-7-yl) ethane-1,2-diol (63.0 mg, yield 54%) as a yellow solid. LC-MS (ESI): mass calced for: C₉H₁₁ClN₄O₂, 242.06; m/z found, 243.1 [M+H]⁺.

### Step E: 3-amino-5-chloro-1-methyl-1H-pyrazolo[4,3-b]pyridine-7-carbaldehyde

To a solution of 1-(3-amino-5-chloro-1-methyl-1H-pyrazolo[4,3-b]pyridin-7-yl) ethane-1,2-diol (63.0 mg, 260 μmol, 1.0 eq) in THF (6.00 mL) and Water (3.00 mL) was added with sodium metaperiodate (83.3 mg, 389 μmol, 1.5 eq). The reaction mixture was stirred at room temperature for 2 h. The mixture was quenched with saturated aqueous Na₂SO₃ solution (30 mL) and extracted with EA (20 mL×3). The organic layer was washed with brine (20 mL), dried over Na₂SO₄, filtered and concentrated under reduced pressure. The residue was purified by Prep-TLC(EA/PE=1/5 v/v) to give 3-amino-5-chloro-1-methyl-1H-pyrazolo[4,3-b]pyridine-7-carbaldehyde (50.0 mg, yield 91%) as a yellow solid. LC-MS (ESI): mass calced for: C₈H₇ClN₄O, 210.03; m/z found, 211.04 [M+H]⁺.

### Intermediate 68:2-chloro-7-methyl-7H-pyrrolo[2,3-d]pyrimidine-4-carbaldehyde

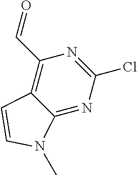

### Step A: 2,4-dichloro-7-methyl-7H-pyrrolo[2,3-d]pyrimidine

To a solution of 2,4-dichloro-7H-pyrrolo[2,3-d]pyrimidine (1.00 g, 5.32 mmol, 1.0 eq) in DMF (20 mL) was added sodium hydride (60% suspend in oil) (0.26 g, 6.38 mmol, 1.2 eq) at 0° C. The mixture was stirred at 0° C. for 0.5 h and MeI (906 mg, 399 μL, 6.38 mmol, 1.2 eq) was added. The resulting mixture was stirred at room temperature for 2 h, diluted with water (20 mL), and extracted with ethyl acetate (20 mL×3). The combined organic layers were washed with brine (15 mL×4), dried over anhydrous Na₂SO₄, filtered and concentrated under reduced pressure. The residue was purified by flash column chromatography on silica gel (ethyl acetate in petroleum ether, from 0% to 10% v/v) to afford 2,4-dichloro-7-methyl-7H-pyrrolo[2,3-d]pyrimidine (1.00 g, yield 93%) as a white solid. LC-MS (ESI): mass calcd.for C₇H₅C₁₂N₃, 202.04; m/z found, 202.1 [M+H]⁺. ¹H NMR (400 MHZ, DMSO-d₆) δ 7.73 (d, J=3.6 Hz, 1H), 6.66 (d, J=3.6 Hz, 1H), 3.80 (s, 3H).

### Step B: 2-chloro-7-methyl-4-vinyl-7H-pyrrolo[2,3-d]pyrimidine

To a solution of 2,4-dichloro-7-methyl-7H-pyrrolo[2,3-d]pyrimidine (600 mg, 2.97 mmol, 1.0 eq) and tributylstannylethylene (1.41 g, 1.30 mL, 4.45 mmol, 1.5 eq) in THF (15.0 mL) was added Bis-(triphenylphosphino)-palladium chloride (104 mg, 148 μmol, 0.05 eq). The mixture was stirred under N₂ at 75° C. overnight. After evaporation, the reaction mixture was diluted with H₂O (100 mL) and extracted with ethyl acetate (40 mL×3). The combined organic layers were washed with brine (15 mL×3), dried over anhydrous Na₂SO₄, filtered and concentrated under reduced pressure. The residue was purified by flash column chromatography on silica gel (ethyl acetate in petroleum ether, from 0% to 10% v/v) to afford 2-chloro-7-methyl-4-vinyl-7H-pyrrolo[2,3-d]pyrimidine (400 mg, yield 79%) as a yellow oil. LC-MS (ESI): mass calcd. for C₉H₈ClN₃,193.63; m/z found, 194.1 [M+H]⁺. ¹H NMR (400 MHz, DMSO-d₆) δ 7.74 (d, J=3.6 Hz, 1H), 7.28-7.23 (m, 1H), 6.99 (d, J=3.6 Hz, 1H), 6.70 (dd, J=17.2, 1.6 Hz, 1H), 5.96 (dd, J=10.8, 1.6 Hz, 1H), 3.88 (s, 3H).

### Step C: 2-chloro-7-methyl-7H-pyrrolo[2,3-d]pyrimidine-4-carbaldehyde

To a solution of 2-chloro-7-methyl-4-vinyl-7H-pyrrolo[2,3-d]pyrimidine (300 mg, 1.55 mmol, 1.0 eq) in Acetone (5.00 mL) and H₂O (5.00 mL) were added with potassium osmate (VI) dihydrate (57.1 mg, 155 μmol, 0.1 eq) and NMO (454 mg, 3.87 mmol, 2.5 eq). The mixture was stirred at room temperature for 1 h, diluted with water (10 mL), and exacted with ethyl acetate (20 mL×3). The organic layer was washed with saturated aqueous NaHCO₃ solution (20 mL) and brine (10 mL×3), dried over anhydrous sodium sulfate, filtered and concentrated under reduced pressure. The residue was dissolved in THF (6.00 mL) and H₂O (3.00 mL), Sodium metaperiodate (497 mg, 2.32 mmol, 1.5 eq) was added. The mixture was stirred under N₂ at room temperature for 2 h. The reaction mixture was quenched with Saturated sodium sulfite solution (2 mL) and with water (5 mL), and extracted with ethyl acetate (10 mL×3). The organic layer was washed with brine (5 mL×3), dried over anhydrous sodium sulfate, filtered and concentrated under reduced pressure. The residue was purified by flash column chromatography on silica gel (ethyl acetate in petroleum ether, from 0% to 50% v/v) to afford 2-chloro-7-methyl-7H-pyrrolo[2,3-d]pyrimidine-4-carbaldehyde (100 mg, yield 33%) as a white solid. LC-MS (ESI): mass calcd. for C₈H₆ClN₃O, 195.61; m/z found, 196.1 [M+1]*. ¹H NMR (400 MHz, DMSO-d₆) δ 10.23 (s, 1H), 8.08 (d, J=3.6 Hz, 1H), 7.17 (d, J=3.6 Hz, 1H), 4.00 (s, 3H).

### Intermediate 69:5-bromo-7-(bromomethyl)-2-methyloxazolo[4,5-b]pyridine (69a) and 5-bromo-2-(bromomethyl)-7-methyloxazolo[4,5-b]pyridine (69b)

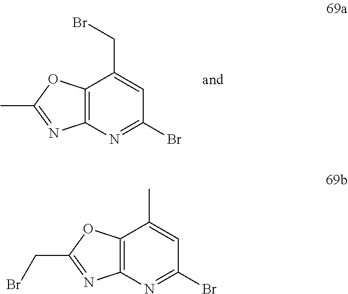

### Step A: 4-methyl-2-nitropyridin-3-ol

To a solution of 4-methylpyridin-3-ol (5.0 g, 45.8 mmol, 1.0 eq) in con. H₂SO₄ (10.0 mL) was dropwise added Fuming Nitric acid (3.5 mL, 45.8 mmol, 1.5 eq) at 0° C. and the mixture was stirred at 0° C. for 2 h. The mixture was quenched with ice water (50 mL), adjusted to pH 6 with aqueous NaOH solution (8 N), and extracted with EA (40 mL×3). The combined organic phases were washed with brine (20 mL×3), dried over anhydrous Na₂SO₄, filtered and concentrated under reduced pressure to give crude 4-methyl-2-nitropyridin-3-ol (6.0 g, yield 85%) as a light yellow solid. LC-MS (ESI): mass calcd. for C₆H₆N₂O₃, 154; m/z found, 155 [M+H]⁺. ¹H NMR (400 MHZ, DMSO-d₆) δ 10.58 (s, 1H), 7.95 (d, J=4.6 Hz, 1H), 7.58 (d, J=4.6 Hz, 1H), 2.33 (s, 3H).

### Step B: 6-bromo-4-methyl-2-nitropyridin-3-ol

To a solution of 4-methyl-2-nitropyridin-3-ol (4.00 g, 26.0 mmol, 1.0 eq) in MeOH (100 mL) was added Sodium methoxide (2.10 g, 38.9 mmol, 1.5 eq). The solution was stirred at room temp for 15 min and then cooled to 0° C. A solution of Br₂ (4.56 g, 1.47 mL, 28.5 mmol, 1.1 eq) in methanol (25 mL) was dropwise added to above mixture and the reaction mixture was stirred at 0° C. for 2 h. After evaporation, the residue was purified by flash column chromatography on silica gel (100% CH₂Cl₂) to obtain 6-bromo-4-methyl-2-nitropyridin-3-ol (4.40 g, yield 73%) as a yellow solid. LC-MS (ESI): mass calcd. for C₆H₅BrN203, 233.02; m/z found, 233.2 [M+H]⁺. ¹H NMR (400 MHZ, DMSO-d₆) δ 10.52 (s, 1H), 7.61 (s, 1H), 2.42 (s, 3H).

### Step C: 2-amino-6-bromo-4-methylpyridin-3-ol

To a solution of 6-bromo-4-methyl-2-nitropyridin-3-ol (3.40 g, 14.6 mmol, 1.0 eq) and ammonium chloride (3.90 g, 2.71 mL, 73.0 mmol, 5.0 eq) in water (20 mL), ethanol (40 mL), and THF (40.0 mL) was added iron powder (4.07 g, 73.0 mmol, 5.0 eq) and the mixture was stirred at 80° C. for 2 h. After cooled to room temperature, the mixture was filtered and the filtrate was concentrated under reduced pressure. The residue was diluted with EA (50 mL), washed with brine (30 mL×2), dried over anhydrous Na₂SO₄, filtered and concentrated under reduced pressure. The residue was purified by flash column chromatography on silica gel (ethyl acetate in petroleum ether, from 0% to 10%) to give 2-amino-6-bromo-4-methylpyridin-3-ol (2.40 g, yield 81%) as a yellow solid. LC-MS (ESI): mass calcd. for C₆H₅BrN203, 203.04; m/z found, 202.9 [M+H]⁺.

### Step D: 5-bromo-2,7-dimethylbenzo[d]oxazole

To a solution of 2-amino-4-bromo-6-methylphenol (2.70 g, 13.4 mmol, 1.0 eq) Triethyl ortho acetate (50.0 mL) was added Acetic acid (1.60 g, 1.54 mL, 26.7 mmol, 2.0 eq) and the mixture was stirred at 120° C. for 16 h. After evaporation, The residue was purified by flash column chromatography on silica gel (ethyl acetate in petroleum ether, from 0% to 30%) to afford 5-bromo-2,7-dimethylbenzo[d]oxazole (2.30 g, yield 76%) as a yellow oil. LC-MS (ESI): mass calcd. for C₉H₈BrNO, 226.07; m/z found, 226.9 [M+H]⁺. ¹H NMR (400 MHZ, DMSO-d₆) δ 7.52 (s, 1H), 2.69 (s, 3H), 2.50 (s, 3H).

### Step E: 5-bromo-7-(bromomethyl)-2-methyloxazolo[4,5-b]pyridine (69a) and 5-bromo-2-(bromomethyl)-7-methyloxazolo[4,5-b]pyridine (69b)

A solution of 5-bromo-2,7-dimethyloxazolo[4,5-b]pyridine (1.00 g, 4.40 mmol, 1.0 eq), NBS (1.41 g, 7.93 mmol, 1.8 eq), and AIBN (145 mg, 881 μmol, 0.2 eq) in CCl₄ (40 mL) was stirred at 80° C. overnight. After cooled to room temperature, the mixture was filtered and the filtrate was concentrated under reduced pressure. The residue was purified by flash column chromatography on silica gel (ethyl acetate in petroleum ether, from 0% to 10%) to 5-bromo-7-(bromomethyl)-2-methyloxazolo[4,5-b]pyridine (800 mg, yield 31%) and 5-bromo-2-(bromomethyl)-7-methyloxazolo[4,5-b]pyridine (180 mg, yield 12%) as a yellow oil. LC-MS (ESI): mass calcd. for C₈H₆Br₂N₂O, 305.96; m/z found, 304.8 [M+H]⁺.

### Intermediate 70:7-(bromomethyl)-5-chloro-2-methyloxazolo [5,4-b]pyridine (70a) and 2-(bromomethyl)-5-chloro-7-methyloxazolo [5,4-b]pyridine (70b)

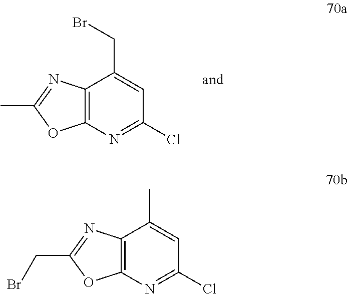

### Step A: 6-chloro-4-methyl-3-nitropyridin-2-ol

To a stirred mixture of 2,6-dichloro-4-methyl-3-nitropyridine (2.0 g, 9.7 mmol, 1.0 eq) in H₂O (25.0 mL) was added NaOH (0.77 g, 19.3 mmol, 2.0 eq). The resulting mixture was stirred at 70° C. for 16 h. The reaction mixture was adjusted to pH 4-5 with diluted aqueous HCl solution (1 N) and extracted with EtOAc (50 mL×3). The combined organic layers were dried over anhydrous Na₂SO₄ and concentrated in vacuum. The residue was purified by flash column chromatography on silica gel (PE/EtOAc=1/1 v/v) to give 6-chloro-4-methyl-3-nitropyridin-2-ol (1.20 g, yield 66%) as a yellow solid. LC-MS (ESI): mass calcd. for C₆H₅ClN₂O₃, 188.0; m/z found, 189.0 [M+H]⁺.

### Step B: 3-amino-6-chloro-4-methylpyridin-2-ol

To a stirred solution of 6-chloro-4-methyl-3-nitropyridin-2-ol (1.1 g, 5.8 mmol, 1.0 eq) in EtOH (8.0 mL), THF (8.0 mL), and H₂O (4.0 mL) were added Iron (1.6 g, 29.2 mmol, 5.0 eq) and Ammonium chloride (1.6 g, 29.2 mmol, 5.0 eq). The reaction mixture was stirred at 80° C. for 2 h. After cooled to room temperature, the reaction mixture was filtered and the cake was washed with MeOH (30 mL×3). The combined filtrates were concentrated to afford 3-amino-6-chloro-4-methylpyridin-2-ol (600 mg, yield 65%) as a brown solid. LC-MS (ESI): mass calcd. for C₆H₇ClN₂O, 158.0; m/z found, 159.0 [M+H]⁺.

### Step C: 5-chloro-2,7-dimethyloxazolo [5,4-b]pyridine

To a stirred solution of 3-amino-6-chloro-4-methylpyridin-2-ol (550 mg, 3.5 mmol, 1.0 eq) in AcOH (10.0 mL) was added Triethyl ortho acetate (563 mg, 635 μL, 3.47 mmol, 1.0 eq). The reaction mixture was stirred under nitrogen atmosphere at 120° C. for 16 h. After cooled to room temperature, the reaction mixture was adjusted to pH 7-8 with diluted aqueous NaHCO₃ solution and extracted with EtOAc (50 mL×3). The combined organic layers were dried over anhydrous Na₂SO₄ and concentrated under reduced pressure. The residue was purified by flash column chromatography on silica gel (PE/EtOAc=1/1 v/v) to give 5-chloro-2,7-dimethyloxazolo [5,4-b]pyridine (430 mg, yield 68%) as a white solid. LC-MS (ESI): mass calcd. for C₈H₇ClN₂O, 182.0; m/z found, 183.0 [M+H]⁺.

### Step D: 7-(bromomethyl)-5-chloro-2-methyloxazolo [5,4-b]pyridine (70a) and 2-(bromomethyl)-5-chloro-7-methyloxazolo [5,4-b]pyridine (70b)

To a stirred mixture of 5-chloro-2,7-dimethyloxazolo [5,4-b]pyridine (330 mg, 1.8 mmol, 1.0 eq) in CCl₄ (10.0 mL) were added AIBN (593 mg, 3.6 mmol, 2.0 eq) and NBS (482 mg, 2.7 mmol, 1.5 eq). The resulting mixture was stirred under nitrogen atmosphere at 80° C. for 2 h. After cooled to room temperature, the reaction mixture was quenched with saturated aqueous Na₂S₂O₃ solution (30 mL) and extracted with EtOAc (50 mL×3). The combined organic layers were washed with H₂O (50 mL), dried over anhydrous Na₂SO₄ and concentrated under reduced pressure. The residue was purified by flash column chromatography on silica gel (PE/EtOAc=1/1 v/v) to give 7-(bromomethyl)-5-chloro-2-methyloxazolo [5,4-b]pyridine (5a) (70.0 mg, yield 15%) as a white solid and 2-(bromomethyl)-5-chloro-7methyloxazolo [5,4-b]pyridine (5b) (70.0 mg, yield 15%) as a white solid. LC-MS (ESI): mass calcd. for C₈H₆BrClN₂O, 259.9; m/z found, 260.9 [M+H]⁺.

### Intermediate 71:5-chloro-3-(ethylamino)-1-((2-(trimethylsilyl)ethoxy)methyl)-1H-pyrazolo[4,3-b]pyridine-7-carbaldehyde

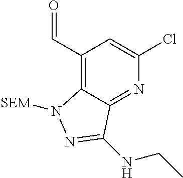 [SMILES: CCNc1nn(COCC[Si](C)(C)C)c2c(C=O)cc(Cl)nc12]

### Step A: 7-bromo-5-chloro-N-ethyl-1-((2-(trimethylsilyl)ethoxy)methyl)-1H-pyrazolo[4,3-b]pyridin-3-amine

To a stirred solution of 7-bromo-5-chloro-1-((2-(trimethylsilyl)ethoxy)methyl)-1H-pyrazolo[4,3-b]pyridin-3-amine (1.00 g, 2.65 mmol, 1.0 eq) in DCM (10.0 mL) was added acetaldehyde (198 mg, 4.50 mmol, 1.7 eq) and AcOH (0.1 mL). The reaction mixture was stirred at 25° C. for 16 h. Then Sodium cyanoborohydride (416 mg, 6.62 mmol, 2.5 eq) was added to above mixture in portions and the resulting mixture was stirred at 25° C. for 4 h. The reaction mixture was filtered and the filtrate was concentrated under reduced pressure. The residue was purified by flash column chromatography on silica gel (PE/EtOAc=10/1 v/v) to obtain 7-bromo-5-chloro-N-ethyl-1-((2-(trimethylsilyl)ethoxy)methyl)-1H-pyrazolo[4,3-b]pyridin-3-amine (300 mg, yield 28%) as a green oil. LC-MS (ESI): mass calced for: C₁₄H₂₂BrClN₄OSi, 404.04; m/z found, 405.0 [M+H]⁺.

### Step B: 5-chloro-N-ethyl-1-((2-(trimethylsilyl)ethoxy)methyl)-7-vinyl-1H-pyrazolo[4,3-b]pyridin-3-amine

To a stirred solution of 7-bromo-5-chloro-N-ethyl-1-((2-(trimethylsilyl)ethoxy)methyl)-1H-pyrazolo[4,3-b]pyridin-3-amine (750 mg, 1.85 mmol, 1.0 eq) and potassium trifluoro (vinyl) borate (322 mg, 2.40 mmol, 1.3 eq) in 1,4-Dioxane (12.0 mL) and Water (1.80 mL) were added Potassium phosphate tribasic (1.18 g, 5.54 mmol, 3.0 eq) and 1,1′-Bis(diphenylphosphino) ferrocene-palladium (II) dichloride (676 mg, 924 μmol, 0.5 eq). The reaction mixture was stirred under nitrogen atmosphere at 80° C. for 8 h. After cooled to room temperature, the reaction mixture was filtered and the filtrate was concentrated under reduced pressure. The residue was purified by flash column chromatography on silica gel (PE/EtOAc=10/1 v/v) to obtain 5-chloro-N-ethyl-1-((2-(trimethylsilyl)ethoxy)methyl)-7-vinyl-1H-pyrazolo[4,3-b]pyridin-3-amine (200 mg, yield 30%) as a yellow oil. LC-MS (ESI): mass calced for: C₁₆H₂₅ClN₄OSi, 352.15; m/z found, 353.1 [M+H]⁺.

### Step C: 5-chloro-3-(ethylamino)-1-((2-(trimethylsilyl)ethoxy)methyl)-1H-pyrazolo[4,3-b]pyridine-7-carbaldehyde

To a stirred solution of 5-chloro-N-ethyl-1-((2-(trimethylsilyl)ethoxy)methyl)-7-vinyl-1H-pyrazolo[4,3-b]pyridin-3-amine (330 mg, 935 μmol, 1.0 eq) in Acetone (8.00 mL) and Water (4.00 mL) were added potassium osmate (VI) dihydrate (172 mg, 468 μmol, 0.5 eq) and N-methylmorpholine N-oxide (274 mg, 2.34 mmol, 2.5 eq) at room temperature. The reaction mixture was stirred under nitrogen atmosphere at 25° C. for 1 h. Then Sodium metaperiodate (1.00 g, 4.68 mmol, 5.0 eq) was added to above mixture and the resulting mixture was stirred at 25° C. for 20 min. The reaction mixture was quenched with water (30 mL) and extracted with EtOAc (40 mL×4). The organic layer was washed with brine (50 mL×2), dried over anhydrous Na₂SO₄, filtered and concentrated under reduced pressure. The residue was purified by flash column chromatography on silica gel (PE/EtOAc=10/1 v/v) to obtain 5-chloro-3-(ethylamino)-1-((2-(trimethylsilyl)ethoxy)methyl)-1H-pyrazolo[4,3-b]pyridine-7-carbaldehyde (206 mg, yield 62%) as a red oil. LC-MS (ESI): mass calced for: C₁₅H₂₃ClN₄O₂Si, 354.13; m/z found, 355.1 [M+H]⁺.

### Intermediate 72:6-chloro-4-(pyrrolidin-1-ylmethyl)-1-((2-(trimethylsilyl)ethoxy)methyl)-1,3-dihydro-2H-pyrrolo[2,3-b]pyridin-2-one

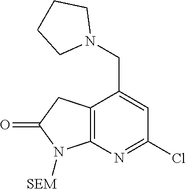 [SMILES: C[Si](C)(C)CCOCN1C(=O)Cc2c(CN3CCCC3)cc(Cl)nc21]

### Step A: methyl 6-chloro-1-((2-(trimethylsilyl)ethoxy)methyl)-1H-pyrrolo[2,3-b]pyridine-4-carboxylate

To a solution of methyl 6-chloro-1H-pyrrolo[2,3-b]pyridine-4-carboxylate (500 mg, 2.37 mmol, 1.0 eq) in DMF (5.00 mL) was added NaH (60% suspend in oil) (68 mg, 2.85 mmol, 1.2 eq) at 0° C. and the mixture was stirred for 30 min. SEM-C₁ (435 mg, 462 μL, 2.61 mmol, 1.1 eq) was added dropwise to above mixture at 0° C. and the mixture was stirred at 25° C. for 2 h. The mixture was quenched with saturated aqueous NH₄Cl solution (30.00 mL) and extracted with EA (20 mL×3). The organic layer was washed with brine (20 mL×4), dried over anhydrous Na₂SO₄, filtered and concentrated under reduced pressure. The crude product was purified by flash column chromatography on silica gel (ethyl acetate in petroleum ether, 9% v/v) to obtain methyl 6-chloro-1-((2-(trimethylsilyl)ethoxy)methyl)-1H-pyrrolo[2,3-b]pyridine-4-carboxylate (650 mg, yield 80%) as a colorless oily. LC-MS (ESI): mass calced for: C₁₅H₂₁ClN₂O₃Si, 340.88; m/z found, 341.1 [M+H]⁺. ¹H NMR (400 MHZ, DMSO-d₆) δ 8.02 (d, J=3.6 Hz, 1H), 7.74 (s, 1H), 7.06 (d, J=3.6 Hz, 1H), 5.74 (s, 2H), 4.07 (s, 3H), 3.65-3.60 (m, 2H), 0.96-0.91 (m, 2H),-0.00 (s, 9H).

### Step B: methyl 3,3-dibromo-6-chloro-2-oxo-1-((2-(trimethylsilyl)ethoxy)methyl)-2,3-dihydro-1H-pyrrolo[2,3-b]pyridine-4-carboxylate

To a solution of methyl 6-chloro-1-((2-(trimethylsilyl)ethoxy)methyl)-1H-pyrrolo[2,3-b]pyridine-4-carboxylate (650 mg, 1.91 mmol, 1.0 eq) in t-BuOH (15.0 mL) was added NBS (1.70 g, 9.53 mmol, 5.0 eq). The mixture was stirred at 30° C. for 16 h. The reaction mixture concentrated in vacuum to give crude product methyl 3,3-dibromo-6-chloro-2-oxo-1-((2-(trimethylsilyl)ethoxy)methyl)-2,3-dihydro-1H-pyrrolo[2,3-b]pyridine-4-carboxylate (980 mg, yield 99%) as a red solid. The crude product was directly used in next step without further purification. LC-MS (ESI): mass calced for: C₁₅H₁₉Br₂ClN₂O₄Si, 514.67; m/z found, 513.1 [M+H]⁺.

### Step C: methyl 6-chloro-2-oxo-1-((2-(trimethylsilyl)ethoxy)methyl)-2,3-dihydro-1H-pyrrolo[2,3-b]pyridine-4-carboxylate

To a solution of methyl 3,3-dibromo-6-chloro-2-oxo-1-((2-(trimethylsilyl)ethoxy)methyl)-2,3-dihydro-1H-pyrrolo[2,3-b]pyridine-4-carboxylate (980 mg, 1.90 mmol, 1.0 eq) in THF (10.0 mL) and saturated ammonium chloride aqueous solution (10 mL) was added zinc (2.49 g, 38.1 mmol, 20.0 eq). The reaction mixture was stirred at 30° C. for 30 min. After filtration, the filtrate is diluted with water (10 mL) and extracted with EA (15 mL×3). The organic layer was washed with brine (30 mL), dried over anhydrous Na₂SO₄, filtered and concentrated under reduced pressure. The crude product was purified by flash column chromatography on silica gel (ethyl acetate in petroleum ether, 13% v/v) to obtain methyl 6-chloro-2-oxo-1-((2-(trimethylsilyl)ethoxy)methyl)-2,3-dihydro-1H-pyrrolo[2,3-b]pyridine-4-carboxylate (370 mg, yield 54%) as a red solid. LC-MS (ESI): mass calced for: C₁₅H₂₁ClN₂O₄Si, 356.88; m/z found, 357.1 [M+H]⁺. ¹H NMR (400 MHZ, DMSO-d₆) δ 7.49 (s, 1H), 5.11 (s, 2H), 3.94 (s, 5H), 3.68-3.61 (m, 2H), 0.94-0.89 (m, 2H),-0.01 (s, 9H).

### Step D: 6-chloro-4-(hydroxymethyl)-1-((2-(trimethylsilyl)ethoxy)methyl)-1,3-dihydro-2H-pyrrolo[2,3-b]pyridin-2-one

To a solution of methyl 6-chloro-2-oxo-1-((2-(trimethylsilyl)ethoxy)methyl)-2,3-dihydro-1H-pyrrolo[2,3-b]pyridine-4-carboxylate (370 mg, 1.04 mmol, 1.0 eq) in THF (7.00 mL) was added LiAlH₄ (78.7 mg, 2.07 mmol, 2.0 eq) at 0° C. and the mixture was stirred for 30 min. The mixture was quench with Na₂SO₄·10H₂O (1 g) at 0° C., filter, and the filtrate was concentrated in vacuum. The crude product was purified by flash column chromatography on silica gel (ethyl acetate in petroleum ether, 40% v/v) to obtain 6-chloro-4-(hydroxymethyl)-1-((2-(trimethylsilyl)ethoxy)methyl)-1,3-dihydro-2H-pyrrolo[2,3-b]pyridin-2-one (180 mg, yield 52%) as a yellow solid. LC-MS (ESI): mass calced for: C₁₄H₂₁ClN₂O₃Si, 328.87; m/z found, 329.1 [M+H]⁺.

### Step E: 6-chloro-4-(pyrrolidin-1-ylmethyl)-1-((2-(trimethylsilyl)ethoxy)methyl)-1,3-dihydro-2H-pyrrolo[2,3-b]pyridin-2-one

To a solution of 6-chloro-4-(hydroxymethyl)-1-((2-(trimethylsilyl)ethoxy)methyl)-1,3-dihydro-2H-pyrrolo[2,3-b]pyridin-2-one (60.0 mg, 182 μmol, 1.0 eq) in DCM (4.00 mL) were added TEA (55.4 mg, 547 μmol, 3.0 eq) and methanesulfonyl chloride (52.2 mg, 35.3 μL, 456 μmol, 2.5 eq) at 0° C. The mixture was stirred at 0° C. for 1 h. Then pyrrolidine (64.9 mg, 912 μmol, 5.0 eq) was added to above mixture and the resulting reaction mixture was stirred at 30° C. for 2 h. After evaporation, the crude product was purified by Prep-TLC(DCM/MeOH=15/1 v/v) to obtain 6-chloro-4-(pyrrolidin-1-ylmethyl)-1-((2-(trimethylsilyl)ethoxy)methyl)-1,3-dihydro-2H-pyrrolo[2,3-b]pyridin-2-one (15.0 mg, yield 21%) as a yellow oil. LC-MS (ESI): mass calced for: C₁₈H₂₈ClN₃O₂Si, 381.98; m/z found, 382.2 [M+H]⁺.

### Intermediate 73:5-chloro-3-cyclopropyl-1-((2-(trimethylsilyl)ethoxy)methyl)-1H-pyrazolo[4,3-b]pyridine-7-carbaldehyde

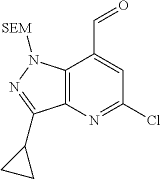 [SMILES: C[Si](C)(C)CCOCn1nc(C2CC2)c2nc(Cl)cc(C=O)c21]

### Step A: 5-chloro-7-vinyl-1H-pyrazolo[4,3-b]pyridine

To a mixture of 7-bromo-5-chloro-1H-pyrazolo[4,3-b]pyridine (5.00 g, 21.5 mmol, 1.0 eq), potassium trifluoro (vinyl) borate (3.46 g, 25.8 mmol, 1.2 eq), and sodium carbonate (5.70 g, 53.8 mmol, 2.5 eq) in 1,4-Dioxane (50.0 mL) and Water (10.0 mL) was added Pd(Ph₃P)₄ (1.24 g, 1.08 mmol, 0.05 eq). The mixture was stirred under N₂ at 100° C. for 16 h. After cooled to room temperature, the mixture was quenched with water (50 mL) and extracted with ethyl acetate (100 mL×3). The combined extracts were washed with water (50 mL) and brine (50 mL), dried over anhydrous sodium sulfate, filtered and concentrated under reduced pressure. The residue was purified with flash column chromatography on silica gel (PE in EA, from 0% to 40% v/v) to afford 5-chloro-7-vinyl-1H-pyrazolo[4,3-b]pyridine (2.1 g, yield 54%) as an off-white solid. LC-MS (ESI): mass calced for C₈H₆ClN₃:179.03; m/z found, 180.0 [M+H]⁺.

### Step B: 5-chloro-3-iodo-7-vinyl-1H-pyrazolo[4,3-b]pyridine

To a solution of 5-chloro-7-vinyl-1H-pyrazolo[4,3-b]pyridine (2.10 g, 11.7 mmol, 1.0 eq) in DMF (20.0 mL) were added potassium hydroxide (1.18 g, 21.0 mmol, 1.8 eq) and diiodine (4.45 g, 17.5 mmol, 1.5 eq). The mixture was stirred at room temperature for 2 h. The mixture was quenched with water (50 mL) and extracted with ethyl acetate (50 mL×3). The combined extracts were washed with aqueous sodium thiosulfate (30 mL×3) and brine (30 mL), dried over anhydrous sodium sulfate, filtered and concentrated under reduced pressure. The residue was purified with flash column chromatography on silica gel (PE in EA, from 20% to 40% v/v) to afford 5-chloro-3-iodo-7-vinyl-1H-pyrazolo[4,3-b]pyridine (2.20 g, yield 62%) as a light yellow solid. LC-MS (ESI): mass calced for C₈H₅ClIN3:304.92; m/z found, 306.1 [M+H]⁺.

### Step C: 5-chloro-3-iodo-1-((2-(trimethylsilyl)ethoxy)methyl)-7-vinyl-1H-pyrazolo[4,3-b]pyridine

To a solution of 5-chloro-3-iodo-7-vinyl-1H-pyrazolo[4,3-b]pyridine (2.20 g, 7.20 mmol, 1.0 eq) in DMF (20.0 mL) was added NaH (60% suspend in oil) (0.52 g, 13.0 mmol, 1.8 eq) at 0° C. and the mixture was stirred under N₂ at 0° C. for 20 min. Then SEMCI (1.80 g, 10.8 mmol, 1.5 eq) was added dropwise to above mixture and the mixture was stirred under N₂ at 0° C. for 1 h. The mixture was quenched with ice-water (50 mL) and extracted with ethyl acetate (50 mL×3). The combined extracts were washed with brine (40 mL×4), dried over anhydrous sodium sulfate, filtered and concentrated under reduced pressure. The residue was purified with flash column chromatography on silica gel (PE in EA, from 0% to 20% v/v) to afford 5-chloro-3-iodo-1-((2-(trimethylsilyl)ethoxy)methyl)-7-vinyl-1H-pyrazolo[4,3-b]pyridine (1.30 g, yield 41%) as a colorless oil. LC-MS (ESI): mass calced for C₁₄H₁₉ClIN3OSi: 435.00; m/z found, 436.2 [M+H]⁺.

### Step D: 5-chloro-3-cyclopropyl-1-((2-(trimethylsilyl)ethoxy)methyl)-7-vinyl-1H-pyrazolo[4,3-b]pyridine

To a stirred mixture of 5-chloro-3-iodo-1-((2-(trimethylsilyl)ethoxy)methyl)-7-vinyl-1H-pyrazolo[4,3-b]pyridine (500 mg, 1.15 mmol, 1.0 eq) and cyclopropylboronic acid (148 mg, 1.72 mmol, 1.5 eq) in 1,4-Dioxane (5.0 mL) and H₂O (1.0 mL) were added Cs₂CO₃ (1.12 g, 3.44 mmol, 3.0 eq) and PdCl₂ (dppf) (84.0 mg, 115 μmol, 0.1 eq). The resulting mixture was stirred under N₂ at 80° C. for 5 h. The reaction mixture was cooled to room temperature, diluted with H₂O (50 mL), and extracted with EtOAc (50 mL×3). The combined organic layers were washed with brine (50 mL×2), dried over anhydrous sodium sulfate, filtered and concentrated in vacuum. The residue was purified by flash column chromatography on silica gel (PE in EA, from 0% to 30% v/v) to give 5-chloro-3-cyclopropyl-1-((2-(trimethylsilyl)ethoxy)methyl)-7-vinyl-1H-pyrazolo[4,3-b]pyridine (130 mg, yield 32%) as a yellow oil. LC-MS (ESI): mass calced for C₁₇H₂₄ClN₃OSi: 349.14; m/z found, 350.0 [M+H]⁺.

### Step E: 5-chloro-3-cyclopropyl-1-((2-(trimethylsilyl)ethoxy)methyl)-1H-pyrazolo[4,3-b]pyridine-7-carbaldehyde

To a stirred mixture of 5-chloro-3-cyclopropyl-1-((2-(trimethylsilyl)ethoxy)methyl)-7-vinyl-1H-pyrazolo[4,3-b]pyridine (70.0 mg, 200 μmol, 1.0 eq) in acetone (3.0 mL) and H₂O (3.0 mL) were added NMO (117 mg, 1.00 mmol, 5.0 eq) and potassium osmate (VI) dihydrate (369 mg, 1.00 mmol, 5.0 eq). The resulting mixture was stirred at room temperature for 1 h. Sodium metaperiodate (214 mg, 1.00 mmol, 5.0 eq) was added to above mixture and the resulting mixture was stirred at room temperature for 1 h. The reaction mixture was diluted with H₂O (50 mL) and extracted with EtOAc (50 mL×3). The combined organic layers were washed with saturated aqueous Na₂S₂O₃ solution (50 mL) and brine (50 mL), dried over anhydrous sodium sulfate, filtered and concentrated in vacuum. The residue was purified by flash column chromatography on silica gel (PE in EA, from 0% to 15% v/v) to give 5-chloro-3-cyclopropyl-1-((2-(trimethylsilyl)ethoxy)methyl)-1H-pyrazolo[4,3-b]pyridine-7-carbaldehyde (30.0 mg, yield 42%) as a yellow oil. LC-MS (ESI): mass calced for C₁₆H₂₂ClN₃O₂Si: 351.12; m/z found, 352.1 [M+H]⁺.

### Intermediate 74:5-chloro-3-(oxetan-3-ylamino)-1-((2-(trimethylsilyl)ethoxy)methyl)-1H-pyrazolo[4,3-b]pyridine-7-carbaldehyde

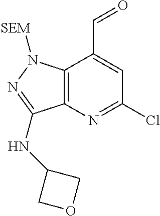 [SMILES: C[Si](C)(C)CCOCn1nc(NC2COC2)c2nc(Cl)cc(C=O)c21]

### Step A: 7-bromo-5-chloro-N-(oxetan-3-yl)-1-((2-(trimethylsilyl)ethoxy)methyl)-1H-pyrazolo[4,3-b]pyridin-3-amine

To a solution of 7-bromo-5-chloro-1-((2-(trimethylsilyl)ethoxy)methyl)-1H-pyrazolo[4,3-b]pyridin-3-amine (1.00 g, 2.65 mmol, 1.0 eq) in MeOH (20.0 mL) were added AcOH (1.05 g, 1.00 mL, 17.5 mmol, 6.60 eq) and MgSO₄ (5.00 g, 40.5 mmol, 15.3 eq) and the mixture was stirred at room temperature for 18 h. Sodium cyanoborohydride (250 mg, 3.97 mmol, 1.5 eq) was added to above mixture and the mixture was stirred under N₂ at room temperature for another 18 h. The solution was diluted with H₂O (10 mL) and extracted with ethyl acetate (30 mL×3). The combined organic layers were washed with brine (20 mL×3), dried over anhydrous Na₂SO₄, filtered and concentrated under reduced pressure. The residue was purified by flash column chromatography on silica gel (Ethyl acetate in petroleum ether, from 0% to 35% v/v) to afford 7-bromo-5-chloro-N-(oxetan-3-yl)-1-((2-(trimethylsilyl)ethoxy)methyl)-1H-pyrazolo[4,3-b]pyridin-3-amine (1.00 g, yield 87%) as a white solid. LC-MS (ESI): mass calced for: C₁₅H₂₂BrClN₄O₂Si, 432.04; m/z found, 433.1 [M+H]⁺. ¹H NMR (400 MHZ, DMSO-d₆) δ 7.89 (s, 1H), 7.30 (d, J=5.0 Hz, 1H), 5.68 (s, 2H), 4.78 (s, 2H), 4.59 (s, 2H), 3.50 (t, J=7.8 Hz, 2H), 0.75 (t, J=7.8 Hz, 2H),-0.13 (s, 9H).

### Step B: 5-chloro-N-(oxetan-3-yl)-1-((2-(trimethylsilyl)ethoxy)methyl)-7-vinyl-1H-pyrazolo[4,3-b]pyridin-3-amine

To a mixture of 7-bromo-5-chloro-N-(oxetan-3-yl)-1-((2-(trimethylsilyl)ethoxy)methyl)-1H-pyrazolo[4,3-b]pyridin-3-amine (100 mg, 231 μmol, 1.0 eq) and Potassium vinyl trifluoroborate (37.1 mg, 277 μmol, 1.2 eq) in dioxane (10.0 mL) and H₂O (1.00 mL) were added 1,1′-Bis(diphenylphosphino) ferrocene-palladium (II) dichloride (84.3 mg, 115 μmol, 0.5 eq) and Potassium phosphate tribasic (147 mg, 692 μmol, 3.0 eq). The mixture was stirred under N₂ at 80° C. for 18 h. The reaction mixture was cooled to room temperature and filtered. The filtrate was concentrated and the residue was purified by flash column chromatography on silica gel (ethyl acetate in Petroleum ether from 0% to 40% v/v) to afford 5-chloro-N-(oxetan-3-yl)-1-((2-(trimethylsilyl)ethoxy)methyl)-7-vinyl-1H-pyrazolo[4,3-b]pyridin-3-amine (50.0 mg, yield 57%) as a yellow oil. LC-MS (ESI): mass calced for: C₁₇H₂₅ClN₄O₂Si, 380.14; m/z found, 381.0 [M+H]⁺.

### Step C: 5-chloro-3-(oxetan-3-ylamino)-1-((2-(trimethylsilyl)ethoxy)methyl)-1H-pyrazolo[4,3-b]pyridine-7-carbaldehyde

To a stirred mixture of 5-chloro-N-(oxetan-3-yl)-1-((2-(trimethylsilyl)ethoxy)methyl)-7-vinyl-1H-pyrazolo[4,3-b]pyridin-3-amine (200 mg, 525 μmol, 1.0 eq) in acetone (10 mL) and Water (6.00 mL) were added potassium osmate (VI) dihydrate (58.0 mg, 158 μmol, 0.3 eq) and NMO (185 mg, 1.58 mmol, 3.0 eq). The resulting mixture was stirred at room temperature for 1 h. Sodium metaperiodate (561 mg, 139 μL, 2.63 mmol, 5.0 eq) was added to above mixture and the resulting mixture was stirred at room temperature for 1 h. The reaction mixture was diluted with H₂O (50 mL) and extracted with EtOAc (50 mL×3). The combined organic layers were washed with brine (50 mL×3), dried over anhydrous Na₂SO₄ and concentrated. The residue was purified by flash column chromatography on silica gel (PE/EtOAc=1/1 v/v) to give 5-chloro-3-(oxetan-3-ylamino)-1-((2-(trimethylsilyl)ethoxy)methyl)-1H-pyrazolo[4,3-b]pyridine-7-carbaldehyde (80.0 mg, yield 40%) as a yellow oil. LC-MS (ESI): mass calced for: C₁₆H₂₃ClN₄O₃Si, 382.12; m/z found, 383.1 [M+H]⁺.

### Intermediate 75:7-(bromomethyl)-5-chloro-3-cyclobutyl-3H-imidazo[4,5-b]pyridine

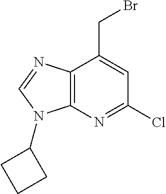 [SMILES: Clc1cc(CBr)c2ncn(C3CCC3)c2n1]

### Step A: 6-chloro-N-cyclobutyl-4-methyl-3-nitropyridin-2-amine

To a solution of 2,6-dichloro-4-methyl-3-nitropyridine (3.00 g, 14.5 mmol, 1.0 eq) and N-ethyl-N-isopropylpropan-2-amine (5.62 g, 43.5 mmol, 3.0 eq) in DMF (30.0 mL) was added cyclobutanyl-amine (1.03 g, 14.5 mmol, 1.0 eq) at −40° C. under N₂ atmosphere and the reaction mixture was stirred at room temperature for 16 h. After evaporation, the residue was purified by flash column chromatography on silica gel (ethyl acetate in petroleum ether, 16% v/v) to obtain 6-chloro-N-cyclobutyl-4-methyl-3-nitropyridin-2-amine (2.90 g, yield 82%) as a yellow oil. LC-MS (ESI): mass calced for: C₁₀H₁₂ClN₃O₂, 241.68; m/z found, 242.1 [M+H]⁺. ¹H NMR (400 MHZ, DMSO-d₆) δ 7.80 (d, J=6.8 Hz, 1H), 6.72 (s, 1H), 4.52-4.36 (m, 1H), 2.34 (s, 3H), 2.32-2.19 (m, 2H), 2.13-1.97 (m, 2H), 1.72-1.63 (m, 2H).

### Step B: 6-chloro-N₂-cyclobutyl-4-methylpyridine-2,3-diamine

To a solution of 6-chloro-N-cyclobutyl-4-methyl-3-nitropyridin-2-amine (2.90 g, 12.0 mmol, 1.0 eq) in THF (12.0 mL), EtOH (12.0 mL), and H₂O (6.00 mL) were added ammonium chloride (3.21 g, 60.0 mmol, 5.0 eq) and iron (3.35 g, 60.0 mmol, 5.0 eq). The reaction was stirred at 80° C. for 2 h, cooled to room temperature, and filtered. The filtrate was concentrated under reduced pressure. The residue was diluted with EA (100 mL), washed with H₂O (50 mL) and brine (50 mL), dried over anhydrous sodium sulfate, filtered and concentrated in vacuum. The crude product was purified by flash column chromatography on silica gel (ethyl acetate in petroleum ether, 33% v/v) to obtain 6-chloro-N₂-cyclobutyl-4-methylpyridine-2,3-diamine (2.20 g, yield 86%) as a yellow oil. LC-MS (ESI): mass calced for: C₁₀H₁₄ClN₃, 211.69; m/z found, 212.1 [M+H]⁺.

### Step C: 5-chloro-3-cyclobutyl-7-methyl-3H-imidazo[4,5-b]pyridine

To a solution of 6-chloro-N₂-cyclobutyl-4-methylpyridine-2,3-diamine (2.20 g, 10.4 mmol, 1.0 eq) in trimethoxymethane (100 mL) was added formic acid (5.00 mL) and the reaction mixture was stirred at 90° C. for 2 h. The reaction mixture was cooled to room temperature and concentrated in vacuum. The residue was diluted with EA (50 mL), washed with saturated aqueous NaHCO₃ solution (50 mL×2) and brine (50 mL), dried over anhydrous sodium sulfate, filtered and concentrated in vacuum. The crude product was purified by flash column chromatography on silica gel (ethyl acetate in petroleum ether, 50% v/v) to obtain 5-chloro-3-cyclobutyl-7-methyl-3H-imidazo[4,5-b]pyridine (2.00 g, yield 86%) as a yellow oil. LC-MS (ESI): mass calced for: C₁₁H₁₂ClN₃, 221.69; m/z found, 222.1 [M+H]⁺. ¹H NMR (400 MHZ, DMSO-d₆) δ 8.64 (s, 1H), 7.21 (s, 1H), 5.09-5.00 (m, 1H), 2.69-2.57 (m, 2H), 2.56 (s, 3H), 2.49-2.42 (m, 2H), 1.92-1.84 (m, 2H).

### Step D: 7-(bromomethyl)-5-chloro-3-cyclobutyl-3H-imidazo[4,5-b]pyridine

To a solution of 5-chloro-3-cyclobutyl-7-methyl-3H-imidazo[4,5-b]pyridine (330 mg, 1.49 mmol, 1.0 eq) in CCl₄ (2.00 mL) were added NBS (291 mg, 1.64 mmol, 1.1 eq) and BPO (36.1 mg, 149 μmol, 0.1 eq). The mixture was stirred at 90° C. for 2 h. The reaction mixture was cooled to room temperature and concentrated in vacuum. The residue was diluted with EA (30 mL), washed with saturated aqueous Na₂S₂O₃ solution (20 mL), water (20 mL) and brine (20 mL), dried over anhydrous sodium sulfate, filtered and concentrated in vacuum. The crude product was purified by Prep-TLC(PE/EA=2/1 v/v) to obtain 7-(bromomethyl)-5-chloro-3-cyclobutyl-3H-imidazo[4,5-b]pyridine (33.0 mg, yield 7%) as a white solid. LC-MS (ESI): mass calced for: C₁₁H₁₁BrClN₃, 298.98; m/z found, 300.1 [M+H]⁺.

### Intermediate 76:5-chloro-1-cyclopropyl-7-(pyrrolidin-1-ylmethyl)-1H-pyrazolo[4,3-b]pyridin-3-amine

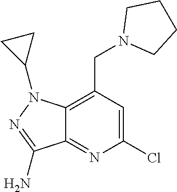 [SMILES: Nc1nn(C2CC2)c2c(CN3CCCC3)cc(Cl)nc12]

### Step A: 7-bromo-5-chloro-1H-pyrazolo[4,3-b]pyridin-3-amine

To a solution of 7-bromo-5-chloro-3-nitro-1H-pyrazolo[4,3-b]pyridine (3.0 g, 10.8 mmol, 1.0 eq) and NH₄Cl (2.9 g, 54.1 mmol, 5.0 eq) in EtOH (15.0 mL), THF (15.0 mL), and H₂O (7.5 mL) was added Fe power (3.0 g, 54.1 mmol, 5.0 eq) at 25° C. The reaction mixture was stirred at 80° C. for 1 h. After cooled to room temperature, the reaction mixture was filtered and the filtrate was concentrated under reduced pressure. The residue was diluted with H₂O (30 mL) and extracted with EA (30 mL×3). The organic layer was washed with brine (30 mL), dried over anhydrous Na₂SO₄, and filtered. The filtrate was concentrated under reduced pressure to get 7-bromo-5-chloro-1H-pyrazolo[4,3-b]pyridin-3-amine (2.5 g, yield 93%) as a yellow solid. The crude product was directly used in next step without further purification. LC-MS (ESI): mass calcd. for C₆H₄BrClN₄, 245.9; m/z found, 246.9 [M+H]⁺.

### Step B: 2-(7-bromo-5-chloro-1H-pyrazolo[4,3-b]pyridin-3-yl) isoindoline-1,3-dione

To a solution of 7-bromo-5-chloro-1H-pyrazolo[4,3-b]pyridin-3-amine (2.5 g, 10.1 mmol, 1.0 eq) in 1,4-dioxane (15.0 mL) was added isobenzofuran-1,3-dione (1.8 g, 12.1 mmol, 1.2 eq) at 25° C. The reaction mixture was stirred at 120° C. for 6 h. After cooled to room temperature, the reaction mixture was quenched with water (50 mL) and extracted with EA (50 mL×3). The organic layer was washed with brine (30 mL), dried over anhydrous Na₂SO₄, filtered and concentrated under reduced pressure. The residue was purified by flash column chromatography on silica gel (PE/EA=2/1 v/v) to give 2-(7-bromo-5-chloro-1H-pyrazolo[4,3-b]pyridin-3-yl) isoindoline-1,3-dione (1.6 g, yield 42%) as a yellow solid. LC-MS (ESI): mass calcd. for C₁₄H₆BrClN₄O₂, 375.9; m/z found, 377.1 [M+H]⁺.

### Step C: 2-(7-bromo-5-chloro-1-cyclopropyl-1H-pyrazolo[4,3-b]pyridin-3-yl) isoindoline-1,3-dione

To a solution of 2-(7-bromo-5-chloro-1H-pyrazolo[4,3-b]pyridin-3-yl) isoindoline-1,3-dione (1.5 g, 4.0 mmol, 1.0 eq) and cyclopropylboronic acid (1.0 g, 12.0 mmol, 3.0 eq) in DCE (15.0 mL) were added Na₂CO₃ (1.3 g, 12.0 mmol, 3.0 eq), 2,2′-bipyridine (620 mg, 4.0 mmol, 1.0 eq), and Cu (OAc)₂ (722 mg, 4.0 mmol, 1.0 eq) at 25° C. The reaction mixture was stirred under O₂ (1 atm) at 75° C. for 6 h. After cooled to room temperature, the reaction mixture filtered, the filtrate was quenched with water (20 mL) and extracted with DCM (30 mL×3). The organic layer was washed with brine (30 mL), dried over anhydrous Na₂SO₄, filtered and concentrated under reduced pressure. The residue was purified by flash column chromatography on silica gel (PE/EA=2/1 v/v) to give 2-(7-bromo-5-chloro-1-cyclopropyl-1H-pyrazolo[4,3-b]pyridin-3-yl) isoindoline-1,3-dione (800 mg, yield 48%) as a yellow solid. LC-MS (ESI): mass calcd. for C₁₇H₁₀BrClN₄O₂, 416.0; m/z found, 417.0 [M+H]⁺.

### Step D: 2-(5-chloro-1-cyclopropyl-7-vinyl-1H-pyrazolo[4,3-b]pyridin-3-yl) isoindoline-1,3-dione

To a solution of 2-(7-bromo-5-chloro-1-cyclopropyl-1H-pyrazolo[4,3-b]pyridin-3-yl) isoindoline-1,3-dione (800 mg, 1.9 mmol, 1.0 eq) in DMF (5.0 mL) were added tributyl (vinyl) stannane (729 mg, 2.3 mmol, 1.2 eq) and Pd (PPh3)₄ (443 mg, 383 μmol, 0.2 eq) at 25° C. The reaction mixture was stirred under N₂ at 75° C. for 2 h. After cooled to room temperature, the reaction mixture was quenched with saturated aqueous KF solution (10 mL), stirred for 1 h, and extracted with EA (10 mL×3). The organic layer was washed with brine (10 mL), dried over anhydrous Na₂SO₄, filtered and concentrated under reduced pressure. The residue was purified by flash column chromatography on silica gel (PE/EA=3/1 v/v) to give 2-(5-chloro-1-cyclopropyl-7-vinyl-1H-pyrazolo[4,3-b]pyridin-3-yl) isoindoline-1,3-dione (500 mg, yield 72%) as a yellow solid. LC-MS (ESI): mass calcd. for C₁₉H₁₃ClN₄O₂, 364.1; m/z found, 365.1 [M+H]⁺.

### Step E: 2-(5-chloro-1-cyclopropyl-7-(1,2-dihydroxyethyl)-1H-pyrazolo[4,3-b]pyridin-3-yl) isoindoline-1,3-dione

To a solution of 2-(5-chloro-1-cyclopropyl-7-vinyl-1H-pyrazolo[4,3-b]pyridin-3-yl) isoindoline-1,3-dione (500 mg, 1.4 mmol, 1.0 eq) in acetone (6.0 mL) and H₂O (6.0 mL) were added K₂OsO₄·2H₂O (101 mg, 274 μmol, 0.2 eq) and NMO (321 mg, 2.74 mmol, 2.0 eq) at 25° C. The reaction mixture was stirred at room temperature for 3 h. The reaction mixture was quenched with water (20 mL) and extracted with DCM (10 mL×3). The organic layer was washed with brine (30 mL), dried over anhydrous Na₂SO₄, filtered and concentrated under reduced pressure. The residue was purified by Prep-TLC(DCM/MeOH=10/1 v/v) to give 2-(5-chloro-1-cyclopropyl-7-(1,2-dihydroxyethyl)-1H-pyrazolo[4,3-b]pyridin-3-yl) isoindoline-1,3-dione (500 mg, yield 92%) as a yellow solid. LC-MS (ESI): mass calcd. for C₁₉H₁₅ClN₄O₄, 398.1; m/z found, 399.1 [M+H]⁺.

### Step F: 5-chloro-1-cyclopropyl-3-(1,3-dioxoisoindolin-2-yl)-1H-pyrazolo[4,3-b]pyridine-7-carbaldehyde

To a solution of 2-(5-chloro-1-cyclopropyl-7-(1,2-dihydroxyethyl)-1H-pyrazolo[4,3-b]pyridin-3-yl) isoindoline-1,3-dione (500 mg, 1.3 mmol, 1.0 eq) in THF (6.0 mL) and H₂O (6.0 mL) was added NaIO₄ (536 mg, 2.5 mmol, 2.0 eq) at 0° C. The reaction mixture was stirred at room temperature for 2 h. The reaction mixture was quenched with saturated aqueous Na₂S₂O₃ solution (20 mL) and extracted with EA (20 mL×3). The organic layer was washed with brine (30 mL), dried over anhydrous Na₂SO₄, filtered and concentrated under reduced pressure. The residue was purified by Prep-TLC(PE/EA=2/1 v/v) to give 5-chloro-1-cyclopropyl-3-(1,3-dioxoisoindolin-2-yl)-1H-pyrazolo[4,3-b]pyridine-7-carbaldehyde (400 mg, yield 87%) as a yellow solid. LC-MS (ESI): mass calcd. for C₁₈H₁₁ClN₄O₃, 366.1; m/z found, 367.1 [M+H]⁺.

### Step G: 2-(5-chloro-1-cyclopropyl-7-(pyrrolidin-1-ylmethyl)-1H-pyrazolo[4,3-b]pyridin-3-yl) isoindoline-1,3-dione

To a solution of 5-chloro-1-cyclopropyl-3-(1,3-dioxoisoindolin-2-yl)-1H-pyrazolo[4,3-b]pyridine-7-carbaldehyde (200 mg, 545 μmol, 1.0 eq) and pyrrolidine (116 mg, 1.6 mmol, 3.0 eq) in DCM (6.0 mL) was added AcOH (0.1 mL) at 25° C. The reaction mixture was stirred at room temperature for 16 h. NaBH(OAc) 3 (116 mg, 545 μmol, 1.0 eq) was added to above mixture and the reaction mixture was stirred at room temperature for 2 h. The reaction mixture was quenched with water (20 mL) and extracted with DCM (10 mL×3). The organic layer was washed with brine (20 mL), dried over Na₂SO₄ and concentrated under reduced pressure. The residue was purified by Prep-TLC(DCM/MeOH=10/1 v/v) to give 2-(5-chloro-1-cyclopropyl-7-(pyrrolidin-1-ylmethyl)-1H-pyrazolo[4,3-b]pyridin-3-yl) isoindoline-1,3-dione (117 mg, yield 51%) as a yellow solid. LC-MS (ESI): mass calcd. for C₂₂H₂OClN₅O₂, 421.1; m/z found, 422.1 [M+H]⁺.

### Step H: 5-chloro-1-cyclopropyl-7-(pyrrolidin-1-ylmethyl)-1H-pyrazolo[4,3-b]pyridin-3-amine

To a solution of 2-(5-chloro-1-cyclopropyl-7-(pyrrolidin-1-ylmethyl)-1H-pyrazolo[4,3-b]pyridin-3-yl) isoindoline-1,3-dione (117 mg, 277 μmol, 1.0 eq) in EtOH (5.0 mL) was added hydrazine hydrate (80% W.t in water) (84.4 μL, 1.4 mmol, 5.0 eq) at 25° C. The reaction mixture was stirred at room temperature for 5 min. The reaction mixture was quenched with water (10 mL) and extracted with EA (10 mL×3). The organic layer was washed with brine (10 mL), dried over anhydrous Na₂SO₄, filtered and concentrated under reduced pressure. The residue was purified by Prep-TLC(DCM/MeOH=10/1 v/v) to give 5-chloro-1-cyclopropyl-7-(pyrrolidin-1-ylmethyl)-1H-pyrazolo[4,3-b]pyridin-3-amine (50.0 mg, yield 62%) as a yellow solid. LC-MS (ESI): mass calcd. for C₁₄H₁₈ClN₅, 291.1; m/z found, 292.1 [M+H]⁺. ¹H NMR (400 MHZ, DMSO-d₆) 7.35 (s, 1H), 5.55 (s, 2H), 4.16 (s, 2H), 3.94-3.88 (m, 1H), 2.64-2.57 (m, 4H), 1.82-1.73 (m, 4H), 1.22-1.17 (m, 2H), 1.08-1.02 (m, 2H).

### Intermediate 77:7-(bromomethyl)-5-chloro-3-(oxetan-3-yl)-3H-imidazo[4,5-b]pyridine

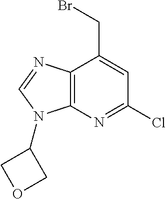 [SMILES: Clc1cc(CBr)c2ncn(C3COC3)c2n1]

### Step A: 6-chloro-4-methyl-3-nitro-N-(oxetan-3-yl) pyridin-2-amine

To a solution of 2,6-dichloro-4-methyl-3-nitropyridine (500 mg, 2.42 mmol, 1.0 eq) in THF (8 mL) was added Diisopropylethylamine (1.56 g, 2.09 mL, 12.1 mmol, 5.0 eq) at room temperature. Then a solution of oxetan-3-amine (177 mg, 2.42 mmol, 1.0 eq) in THF (2 mL) was dropwise added to above mixture at −40° C. and the reaction mixture was stirred at room temperature for 16 h. The mixture was quenched with water (20 mL) and extracted with EtOAc (20 mL×3). The organic layers were washed with brine (20 mL×3), dried over anhydrous Na₂SO₄, filtered and concentrated in vacuum. The crude product was purified by flash column chromatography on silica gel (PE/EA=10/1 v/v) to provide 6-chloro-4-methyl-3-nitro-N-(oxetan-3-yl) pyridin-2-amine (180 mg, yield 31%) as a yellow solid. LC-MS (ESI): mass calced for: C₉H₁₀ClN₃O₃, 243.04; m/z found, 244.1 [M+H]⁺. ¹H NMR (400 MHZ, DMSO-d₆) δ 8.23 (d, J=4.4 Hz, 1H), 6.88 (s, 1H), 4.99-4.97 (m, 1H), 4.80 (t, J=6.8 Hz, 2H), 4.61 (t, J=6.4 Hz, 2H), 2.41 (s, 3H).

### Step B: 6-chloro-4-methyl-N₂-(oxetan-3-yl)pyridine-2,3-diamine

To a solution of 6-chloro-4-methyl-3-nitro-N-(oxetan-3-yl) pyridin-2-amine (1.56 g, 6.40 mmol, 1.0 eq) and Ammonium chloride (1.71 g, 32.0 mmol, 5.0 eq) in THF (30.0 mL), EtOH (30.0 mL), and water (15.0 mL) was added iron (1.79 g, 32.0 mmol, 5.0 eq) at room temperature. The mixture was stirred at 70° C. for 1 h. After cooled to room temperature, the mixture was filtered and the cake was washed with EA (100 mL×3). The filtrate was concentrated and the residue was diluted with EA (60 mL). The organic layer was washed with water (100 mL) and brine (100 mL), dried over anhydrous Na₂SO₄, filtered and concentrated under reduced pressure to give 6-chloro-4-methyl-N₂-(oxetan-3-yl)pyridine-2,3-diamine (1.20 g, yield 88%) as a yellow solid. LC-MS (ESI): mass calced for: C₉H₁₂ClN₃O, 213.07; m/z found, 214.1 [M+H]⁺.

### Step C: 5-chloro-7-methyl-3-(oxetan-3-yl)-3H-imidazo[4,5-b]pyridine

To a solution of 6-chloro-4-methyl-N₂-(oxetan-3-yl)pyridine-2,3-diamine (130 mg, 608 μmol, 1.0 eq) in Trimethyl orthoformate (TMOF) (8.00 mL) was added Formic Acid (0.20 mL) at room temperature. The reaction mixture was stirred at 90° C. for 2 h. After evaporation, the crude product was purified by flash column chromatography on silica gel (100% EA) to provide 5-chloro-7-methyl-3-(oxetan-3-yl)-3H-imidazo[4,5-b]pyridine (74.0 mg, yield 54%) as a yellow solid. LC-MS (ESI): mass calced for: C₁₀H₁₀ClN₃O, 223.05; m/z found, 224.1 [M+H]⁺. ¹H NMR (400 MHZ, DMSO-d₆) δ 8.75 (s, 1H), 7.26 (s, 1H), 5.81-5.73 (m, 1H), 5.09 (t, J=6.8 Hz, 2H), 4.99 (t, J=7.2 Hz, 2H), 2.58 (s, 3H).

### Step D: 7-(bromomethyl)-5-chloro-3-(oxetan-3-yl)-3H-imidazo[4,5-b]pyridine

To a solution of 5-chloro-7-methyl-3-(oxetan-3-yl)-3H-imidazo[4,5-b]pyridine (350 mg, 1.56 mmol, 1.0 eq) and N-bromosuccinimide (306 mg, 1.72 mmol, 1.1 eq) in CCl₄ (20.0 mL) was added Benzoyl peroxide (56.9 mg, 235 μmol, 0.15 eq) at room temperature. The reaction mixture was heating at 90° C. for 3 h. After cooled to room temperature, the mixture was quenched with saturated NaHCO₃ solution (3 0 mL) and extracted with DCM (30 mL×3). The organic layers were washed with brine (30 mL), dried over anhydrous Na₂SO₄, filtered and concentrated in vacuum. The crude product was purified by Prep-TLC(PE/EA=1/2 v/v) to give 7-(bromomethyl)-5-chloro-3-(oxetan-3-yl)-3H-imidazo[4,5-b]pyridine (50.0 mg, yield 11%) as a yellow oil. LC-MS (ESI): mass calced for: C₁₀H₉BrClN₃O, 300.96; m/z found, 301.97 [M+H]⁺.

¹H NMR (400 MHZ, DMSO-d₆) δ 8.87 (s, 1H), 7.52 (s, 1H), 5.85-5.75 (m, 1H), 5.11 (t, J=6.8 Hz, 2H), 5.00 (t, J=7.4 Hz, 2H), 4.95 (s, 2H).

### Intermediate 78:2-chloro-4-(pyrrolidin-1-ylmethyl)-7H-pyrrolo[2,3-d]pyrimidine

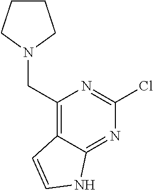 [SMILES: Clc1nc(CN2CCCC2)c2cc[nH]c2n1]

### Step A: 2,4-dichloro-7-((2-(trimethylsilyl)ethoxy)methyl)-7H-pyrrolo[2,3-d]pyrimidine

To a solution of 2,4-dichloro-7H-pyrrolo[2,3-d]pyrimidine (5.00 g, 26.6 mmol, 1.0 eq) in DMF (50.0 mL) was added NaH (60% suspend in oil) (1.60 g, 39.9 mmol, 1.5 eq) in portions at 0° C. and the mixture was stirred for 1 h. Then SEM-C₁ (6.65 g, 7.06 mL, 9.9 mmol, 1.5 eq) was added to above mixture and the reaction mixture was stirred at room temperature for 1 h. The reaction solution was diluted with ethyl acetate (50 mL), quenched by adding saturated aqueous ammonium chloride solution (30 mL), and extracted with EA (50 mL×3). The organic phase was washed with brine (50 mL×3), dried over anhydrous sodium sulfate, and filtered. The filtrate was concentrated under reduced pressure to obtain 2,4-dichloro-7-((2-(trimethylsilyl)ethoxy)methyl)-7H-pyrrolo[2,3-d]pyrimidine (8.30 g, yield 88%) as a yellow oil. LC-MS (ESI): mass calced for: C₁₂H₁₇C₁₂N₃OSi, 317.05; m/z found, No mass signal. ¹H NMR (400 MHZ, DMSO-d₆) δ 7.90 (d, J=3.6 Hz, 1H), 6.78 (d, J=3.6 Hz, 1H), 5.60 (s, 2H), 3.56-3.50 (m, 2H), 0.87-0.81 (m, 2H),-0.09 (s, 9H).

### Step B: 2-chloro-7-((2-(trimethylsilyl)ethoxy)methyl)-4-vinyl-7H-pyrrolo[2,3-d]pyrimidine

To a solution of 2,4-dichloro-7-((2-(trimethylsilyl)ethoxy)methyl)-7H-pyrrolo[2,3-d]pyrimidine (7.30 g, 20.6 mmol, 1.0 eq) and Bis-(triphenylphosphino)-palladous chloride (1.45 g, 2.06 mmol, 0.1 eq) in THF (80.0 mL) was added dibutyl (pentyl) (vinyl) stannane (8.20 g, 7.57 mL, 24.8 mmol, 1.2 eq) and the reaction mixture was stirred under N₂ at 70° C. for 6 h. After cooled to room temperature, the reaction mixture was diluted with ethyl acetate (200 mL) and saturated aqueous potassium fluoride solution (200 mL). The mixture was stirred for 1 h, filtered, and the filtrate was extracted with EA (100 mL×3). The organic phase was washed with brine (150 mL), dried over anhydrous sodium sulfate, filtered and concentrated under reduced pressure. The residue was purified by flash column chromatography on silica gel (ethyl acetate in petroleum ether, from 0% to 10% v/v) to afford 2-chloro-7-((2-(trimethylsilyl)ethoxy)methyl)-4-vinyl-7H-pyrrolo[2,3-d]pyrimidine (3.00 g, yield 47%) as a yellow oil. LC-MS (ESI): mass calced for: C₁₄H₂OClN₃OSi, 309.11; m/z found, 310.2 [M+H]⁺. ¹H NMR (400 MHZ, DMSO-d₆) δ 7.88 (d, J=3.6 Hz, 1H), 7.30 (dd, J=17.2, 10.8 Hz, 1H), 7.08 (d, J=3.6 Hz, 1H), 6.72 (dd, J=17.2, 1.2 Hz, 1H), 5.99 (dd, J=10.8, 1.2 Hz, 1H), 5.67 (s, 2H), 3.68-3.55 (m, 2H), 0.98-0.89 (m, 2H), 0.00 (s, 9H).

### Step C: 2-chloro-7-((2-(trimethylsilyl)ethoxy)methyl)-7H-pyrrolo[2,3-d]pyrimidine-4-carbaldehyde

To a solution of 2-chloro-7-((2-(trimethylsilyl)ethoxy)methyl)-4-vinyl-7H-pyrrolo[2,3-d]pyrimidine (1.00 g, 3.22 mmol, 1.0 eq) in Acetone (10.0 mL) and H₂O (10.0 mL) were added K₂OsO₄·2H₂O (56.0 mg, 322 μmol, 0.1 eq) and NMO (753 mg, 6.43 mmol, 2.0 eq) and the mixture was stirred at room temperature for 4 h. After filtered, the filtrate was concentrated under reduced pressure. The residue was diluted with EA (50 mL), washed with brine (50 mL), dried over anhydrous sodium sulfate, filtered and concentrated under reduced pressure. The residue was dissolved in THF (20 mL) and H₂O (10 mL), sodium periodate (3.44 g, 16.1 mmol, 5.0 eq) was added to above solution. The reaction mixture was stirred at room temperature for 2 h. The reaction mixture was quenched with saturated aqueous Na₂S₂O₃ solution (30 mL) and extracted with EA (40 mL×3). The organic layer was washed with saturated aqueous NaHCO₃ solution (20 mL) and brine (20 mL), dried over anhydrous Na₂SO₄, filtered and concentrated under reduced pressure. The residue was purified by flash column chromatography on silica gel (ethyl acetate in petroleum ether, from 0% to 15% v/v) to give 2-chloro-7-((2-(trimethylsilyl)ethoxy)methyl)-7H-pyrrolo[2,3-d]pyrimidine-4-carbaldehyde (310 mg, yield 31%) as a yellow solid. LC-MS (ESI): mass calced for: C₁₃H₁₈ClN₃O₂Si, 311.09; m/z found, 312.1 [M+H]⁺. ¹H NMR (400 MHZ, DMSO-d₆) δ 10.17 (s, 1H), 8.17 (d, J=3.6 Hz, 1H), 7.18 (d, J=3.6 Hz, 1H), 5.74 (s, 2H), 3.67-3.57 (m, 2H), 1.01-0.88 (m, 2H), 0.00 (s, 9H).

### Step D: 2-chloro-4-(pyrrolidin-1-ylmethyl)-7-((2-(trimethylsilyl)ethoxy)methyl)-7H-pyrrolo[2,3-d]pyrimidine

To a solution of 2-chloro-7-((2-(trimethylsilyl)ethoxy)methyl)-7H-pyrrolo[2,3-d]pyrimidine-4-carbaldehyde (1.10 g, 3.53 mmol, 1.0 eq) in DMF (15.0 mL) were added pyrrolidine (376 mg, 5.29 mmol, 1.5 eq) and AcOH (21.2 mg, 353 μmol, 0.1 eq) and the reaction was stirred at room temperature for 4 h. Then sodium cyanoborohydride (665 mg, 10.6 mmol, 3.0 eq) was added to above mixture and the reaction was stirred at room temperature for 2 h. The reaction solution was diluted with ethyl acetate (50 mL), washed with brine (30 mL×4), dried over anhydrous sodium sulfate, filtered and concentrated under reduced pressure. The residue was purified by flash column chromatography on silica gel (methanol in dichloromethane, from 0% to 5% v/v) to afford 2-chloro-4-(pyrrolidin-1-ylmethyl)-7-((2-(trimethylsilyl)ethoxy)methyl)-7H-pyrrolo[2,3-d]pyrimidine (800 mg, yield 56%) as a yellow oil. LC-MS (ESI): mass calced for: C₁₇H₂₇ClN₄OSi, 366.16; m/z found, 367.1 [M+H]⁺. ¹H NMR (400 MHZ, DMSO-d₆) δ 7.81 (d, J=3.6 Hz, 1H), 6.99 (d, J=3.6 Hz, 1H), 5.67 (s, 2H), 4.06 (s, 2H), 3.69-3.56 (m, 2H), 2.65-2.62 (m, 4H), 1.84-1.80 (m, 4H), 0.98-0.85 (m, 2H), 0.00 (s, 9H).

### Step E: 2-chloro-4-(pyrrolidin-1-ylmethyl)-7H-pyrrolo[2,3-d]pyrimidine

To a solution of 2-chloro-4-(pyrrolidin-1-ylmethyl)-7-((2-(trimethylsilyl)ethoxy)methyl)-7H-pyrrolo[2,3-d]pyrimidine (750 mg, 2.04 mmol, 1.0 eq) in DCM (6.00 mL) was added TFA (2.00 mL) and the reaction mixture was stirred at room temperature for 1 h. After evaporation, the residue was diluted with MeOH (5.00 mL) and NH₄OH (2.00 mL). The resulting reaction mixture was stirred at room temperature for 1 h. The mixture was concentrated under reduced pressure to obtain 2-chloro-4-(pyrrolidin-1-ylmethyl)-7H-pyrrolo[2,3-d]pyrimidine (450 mg, yield 93%) as a yellow solid. The product was directly used in the next step without further purification. LC-MS (ESI): mass calced for: C₁₁H₁₃ClN₄, 236.08; m/z found, 237.3 [M+H]⁺.

### Intermediate 79:5,6-dichloro-4-(pyrrolidin-1-ylmethyl)-1H-pyrrolo[2,3-b]pyridine

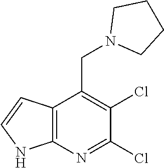 [SMILES: Clc1nc2[nH]ccc2c(CN2CCCC2)c1Cl]

### Step A: (5,6-dichloro-1H-pyrrolo[2,3-b]pyridin-4-yl) methanol

To a solution of methyl 5,6-dichloro-1H-pyrrolo[2,3-b]pyridine-4-carboxylate (250 mg, 1.02 mmol, 1.0 eq) in THF (10.00 mL) was added LiAlH₄ (116 mg, 3.06 mmol, 3.0 eq) in portions at 0° C. The mixture was stirred at 0° C. for 10 min. The reaction mixture was quenched with Na₂SO_{4 10}H₂O (300 mg) at 0° C. and stirred for 10 min. After filtration, the filtrate was concentrated in vacuum to afford a crude product (5,6-dichloro-1H-pyrrolo[2,3-b]pyridin-4-yl) methanol (200 mg, yield 90%) as a yellow solid. The crude product was directly used in next step without further purification. LC-MS (ESI): mass calced for: C₈H₆C₁₂N₂O, 217.05; m/z found, 217.1 [M+H]⁺.

### Step B: 5,6-dichloro-1H-pyrrolo[2,3-b]pyridine-4-carbaldehyde

To a solution of (5,6-dichloro-1H-pyrrolo[2,3-b]pyridin-4-yl) methanol (200 mg, 921 μmol, 1.0 eq) in DMSO (8.00 mL), was added IBX (Purity 45% W.t) (1.15 g, 1.84 mmol, 2.0 eq). The mixture was stirred at 30° C. for 1 h. The mixture was quenched with saturated aqueous Na₂SO₃ solution (20 mL) and extracted with EA (10 mL×3). The organic layer was washed with aqueous NaHCO₃ solution (20 mL), dried over anhydrous Na₂SO₄, filtered and concentrated under reduced pressure. The crude product was purified by Prep-TLC(PE/EA=4/1 v/v) to afford 5,6-dichloro-1H-pyrrolo[2,3-b]pyridine-4-carbaldehyde (180 mg, yield 90%) as a yellow solid. LC-MS (ESI): mass calced for: C₈H₄C₁₂N₂O, 215.03; m/z found, 215.0 [M+H]⁺.

### Step C: 5,6-dichloro-4-(pyrrolidin-1-ylmethyl)-1H-pyrrolo[2,3-b]pyridine

To a solution of 5,6-dichloro-1H-pyrrolo[2,3-b]pyridine-4-carbaldehyde (180 mg, 837 μmol, 1.0 eq) in DCM (6.00 mL) were added AcOH (0.2 mL) and pyrrolidine (119 mg, 1.67 mmol, 2.0 eq). After 1 h Sodium triacetoxyborohydride (266 mg, 1.26 mmol, 1.5 eq) was added to above mixture and the resulting reaction mixture was stirred at 30° C. for 1 h. The reaction mixture was quenched with saturated aqueous NaHCO₃ solution (15 mL) and extracted with DCM (15 mL×3). The combined organic layers were washed with brine (15 mL), dried over anhydrous Na₂SO₄, filtered and concentrated under reduced pressure. The crude product was purified by Prep-TLC(DCM/MeOH=10/1 v/v) to afford 5,6-dichloro-4-(pyrrolidin-1-ylmethyl)-1H-pyrrolo[2,3-b]pyridine (130 mg, yield 57%) as a white solid. LC-MS (ESI): mass calced for: C₁₂H₁₃C₁₂N₃, 270.16; m/z found, 270.2 [M+H]⁺.

### Intermediate 80:5-chloro-3-cyclobutyl-1-(tetrahydro-2H-pyran-2-yl)-1H-pyrazolo[4,3-b]pyridine-7-carbaldehyde

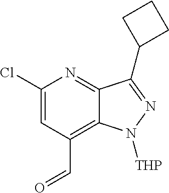

### Step A: (6-chloro-3-fluoropyridin-2-yl) (cyclobutyl) methanol

To a solution of 6-chloro-3-fluoropicolinaldehyde (3.5 g, 21.9 mmol, 1.0 eq) in THF (30 mL) was added dropwise cyclobutyl magnesium bromide (2 M in THF) (16.5 mL, 32.9 mmol, 1.5 eq) at 0° C. The reaction mixture was stirred at 0° C. for 1 h, quenched with water (20 mL), and extracted with EA (30 mL×3). The organic layer was washed with brine (30 mL), dried over anhydrous Na₂SO₄, filtered and concentrated under reduced pressure. The residue was purified by flash column chromatography on silica gel (PE/EA=10/1 v/v) to give (6-chloro-3-fluoropyridin-2-yl) (cyclobutyl) methanol (1.5 g, yield 32%) as a yellow oil. LC-MS (ESI): mass calcd. for C₁₀H₁₁CIFNO, 215.1; m/z found, 216.1 [M+H]⁺. ¹H NMR (400 MHZ, DMSO-d₆) δ 7.78 (t, J=8.8 Hz, 1H), 7.47 (dd, J=8.8, 3.2 Hz, 1H), 5.39 (d, J=6.2 Hz, 1H), 4.77-4.64 (m, 1H), 2.79-2.69 (m, 1H), 2.80-1.93 (m, 2H), 1.85-1.57 (m, 4H).

### Step B: (6-chloro-3-fluoropyridin-2-yl) (cyclobutyl) methanone

To a solution of (6-chloro-3-fluoropyridin-2-yl) (cyclobutyl) methanol (390 mg, 1.8 mmol, 1.0 eq) in DMSO (8.0 mL) was added IBX (506 mg, 1.8 mmol, 1.0 eq) at 25° C. The reaction mixture was stirred at room temperature for 12 h. The reaction mixture was quenched with water (30 mL) and extracted with DCM (30 mL×3). The organic layer was washed with brine (30 mL×3), dried over anhydrous Na₂SO₄, filtered and concentrated under reduced pressure. The residue was purified by flash column chromatography on silica gel (PE/EA=2/1 v/v) to give (6-chloro-3-fluoropyridin-2-yl) (cyclobutyl) methanone (300 mg, yield 78%) as a yellow oil. LC-MS (ESI): mass calcd. for C₁₀H₉CIFNO, 213.0; m/z found, 214.0 [M+H]⁺.

### Step C: 5-chloro-3-cyclobutyl-1H-pyrazolo[4,3-b]pyridine

To a solution of (6-chloro-3-fluoropyridin-2-yl) (cyclobutyl) methanone (300 mg, 1.4 mmol, 1.0 eq) in pyridine (3.0 mL) was added hydrazine hydrate (80% w.t. in water) (855 μL, 14.0 mmol, 10.0 eq) at 25° C. The reaction mixture was stirred under microwave at 120° C. for 2 h. After cooled to room temperature, the reaction mixture was quenched with diluted aqueous HCl solution (3 M) (50 mL) and extracted with EA (10 mL×3). The organic layer was washed with brine (10 mL), dried over anhydrous Na₂SO₄, filtered and concentrated under reduced pressure. The residue was purified by flash column chromatography on silica gel (PE/EA=2/1 v/v) to give 5-chloro-3-cyclobutyl-1H-pyrazolo[4,3-b]pyridine (250 mg, yield 86%) as a yellow solid. LC-MS (ESI): mass calcd. for C₁₀H₁₀ClN₃, 207.1; m/z found, 208.1 [M+H]⁺.

### Step D: 5-chloro-3-cyclobutyl-1-(tetrahydro-2H-pyran-2-yl)-1H-pyrazolo[4,3-b]pyridine

To a solution of 5-chloro-3-cyclobutyl-1H-pyrazolo[4,3-b]pyridine (1.0 g, 4.8 mmol, 1.0 eq) in DCM (12.0 mL) were added p-toluenesulfonic acid (82.9 mg, 482 μmol, 0.1 eq) and 3,4-dihydro-2H-pyran (608 mg, 7.2 mmol, 1.5 eq) at 25° C. The reaction mixture was stirred at room temperature for 16 h. The reaction mixture was quenched with water (10 mL) and extracted with DCM (10 mL×3). The organic layer was washed with saturated aqueous NaHCO₃ solution (30 mL×2) and brine (10 mL), dried over anhydrous Na₂SO₄, filtered and concentrated under reduced pressure. The residue was purified by flash column chromatography on silica gel (PE/EA=5/1 v/v) to give 5-chloro-3-cyclobutyl-1-(tetrahydro-2H-pyran-2-yl)-1H-pyrazolo[4,3-b]pyridine (1.2 g, yield 85%) as a white solid. LC-MS: 292 (M+H)+. Revised as the following: LC-MS (ESI): mass calcd. for C₁₅H₁₈ClN₃O, 291.1; m/z found, 292.1 [M+H]⁺. ¹H NMR (400 MHZ, DMSO-d₆) δ 8.24 (d, J=8.8 Hz, 1H), 7.47 (d, J=8.8 Hz, 1H), 5.83 (dd, J=9.6, 2.2 Hz, 1H), 3.96-3.85 (m, 2H), 3.75-3.70 (m, 1H), 2.53-2.45 (m, 2H), 2.42-2.29 (m, 2H), 2.16-1.93 (m, 4H), 1.79-1.67 (m, 1H), 1.63-1.55 (m, 2H), 1.52-1.42 (m, 1H).

### Step E: 5-chloro-3-cyclobutyl-7-iodo-1-(tetrahydro-2H-pyran-2-yl)-1H-pyrazolo[4,3-b]pyridine

To a solution of 5-chloro-3-cyclobutyl-1-(tetrahydro-2H-pyran-2-yl)-1H-pyrazolo[4,3-b]pyridine (200 mg, 685 μmol, 1.0 eq) in dry THF (3 mL) was added n-Butyllithium (2.5 M in n-hexane) (411 μL, 1.1 mmol, 1.5 eq) under N₂ at −78° C. The reaction mixture was stirred at −78° C. for 0.5 h. A solution of I₂ (261 mg, 1.1 mmol, 1.5 eq) in THF (0.5 mL) was added dropwise to above mixture and the resulting reaction mixture was stirred at −78° C. for 0.5 h. The reaction mixture was quenched with saturated aqueous Na₂S₂O₃ solution (10 mL) and extracted with EA (10 mL×3). The organic layer was washed with brine (10 mL), dried over anhydrous Na₂SO₄, filtered and concentrated under reduced pressure. The residue was purified by flash column chromatography on silica gel (PE/EA=5/1 v/v) to give 5-chloro-3-cyclobutyl-7-iodo-1-(tetrahydro-2H-pyran-2-yl)-1H-pyrazolo[4,3-b]pyridine (138 mg, yield 48%) as a white solid. LC-MS (ESI): mass calcd. for C₁₅H₁₇ClIN₃O, 417.0; m/z found, 418.0 [M+H]⁺. ¹H NMR (400 MHZ, DMSO-d₆) δ 7.97 (s, 1H), 6.26 (d, J=8.4 Hz, 1H), 3.90-3.81 (m, 2H), 3.75-3.66 (m, 1H), 2.52-2.26 (m, 6H), 2.02 (dd, J=18.6, 9.6 Hz, 3H), 1.93-1.83 (m, 1H), 1.71-1.60 (m, 1H), 1.56-1.48 (m, 1H).

### Step F: 5-chloro-3-cyclobutyl-1-(tetrahydro-2H-pyran-2-yl)-7-vinyl-1H-pyrazolo[4,3-b]pyridine

To a solution of 5-chloro-3-cyclobutyl-1-(tetrahydro-2H-pyran-2-yl)-7-vinyl-1H-pyrazolo[4,3-b]pyridine (600 mg, 1.9 mmol, 1.0 eq) in acetone (10.0 mL) and H₂O (5.0 mL) were added potassium osmate (VI) dihydrate (139 mg, 378 μmol, 0.2 eq) and NMO (442 mg, 3.8 mmol, 2.0 eq) at room temperature. The reaction mixture was stirred at 25° C. for 1 h. The reaction mixture was quenched with H₂O (10 mL) and extracted with EA (5 mL×3). The organic layer was washed with brine (10 mL), dried over anhydrous Na₂SO₄, filtered and concentrated under reduced pressure. The residue was purified by Prep-TLC(DCM/MeOH=10/1 v/v) to afford 1-(5-chloro-3-cyclobutyl-1-(tetrahydro-2H-pyran-2-yl)-1H-pyrazolo[4,3-b]pyridin-7-yl) ethane-1,2-diol (500 mg, yield 75%) as a brown solid. LC-MS (ESI): mass calcd. for C₁₇H₂₂ClN₃O₃, 351.1; m/z found, 352.1 [M+H]⁺.

### Step G: 5-chloro-3-cyclobutyl-1-(tetrahydro-2H-pyran-2-yl)-1H-pyrazolo[4,3-b]pyridine-7-carbaldehyde

To a solution of 1-(5-chloro-3-cyclobutyl-1-(tetrahydro-2H-pyran-2-yl)-1H-pyrazolo[4,3-b]pyridin-7-yl) ethane-1,2-diol (500 mg, 1.4 mmol, 1.0 eq) in acetone (5.0 mL) and H₂O (2.5 mL) was added NaIO₄ (608 mg, 2.8 mmol, 2.0 eq) at 25° C. The reaction mixture was stirred at room temperature for 1 h. The reaction mixture was quenched with H₂O (10 mL) and extracted with EA (10 mL×3). The organic layer was washed with brine (10 mL), dried over anhydrous Na₂SO₄, filtered and concentrated under reduced pressure. The residue was purified by flash column chromatography on silica gel (PE/EA=5/1 v/v) to give 5-chloro-3-cyclobutyl-1-(tetrahydro-2H-pyran-2-yl)-1H-pyrazolo[4,3-b]pyridine-7-carbaldehyde (400 mg, yield 88%) as a white solid. LC-MS (ESI): mass calcd. for C₁₆H₁₈ClN₃O₂, 319.1; m/z found, 320.1 [M+H]⁺.

### Intermediate 81:3-(7-(bromomethyl)-5-chloro-3H-imidazo[4,5-b]pyridin-3-yl) thietane 1,1-dioxide

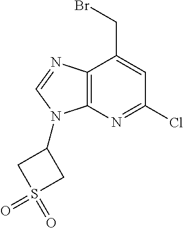 [SMILES: O=S1(=O)CC(n2cnc3c(CBr)cc(Cl)nc32)C1]

### Step A: 6-chloro-4-methyl-3-nitropyridin-2-amine

2,6-dichloro-4-methyl-3-nitropyridine (3.00 g, 14.5 mmol, 1.0 eq) was suspended in NH₃-MeOH (7 M) (100 mL). The reaction was stirred at room temperature for 40 h. After evaporation, the crude was purified by flash column chromatography on silica gel (PE/EA=20/1 v/v) to provide 6-chloro-4-methyl-3-nitropyridin-2-amine (1.54 g, yield 56%) as a yellow solid. LC-MS (ESI): mass calced for: C₆H₆ClN₃O₂, 187.01; m/z found, 188.1 [M+H]⁺. ¹H NMR (400 MHZ, DMSO-d₆) δ 7.58 (s, 2H), 6,71 (s, 1H), 2.37 (s, 3H).

### Step B: 6-chloro-4-methylpyridine-2,3-diamine

To a solution of 6-chloro-4-methyl-3-nitropyridin-2-amine (1.00 g, 5.33 mmol, 10 eq) and Ammonium chloride (1.43 g, 26.7 mmol, 5.0 eq) in EtOH (20.0 mL), THF (20.0 mL), and Water (10.0 mL) was added Iron (1.49 g, 26.7 mmol, 5.0 eq) at room temperature. The mixture was stirred at 70° C. for 1 h. After cooled to room temperature, the mixture was filtered and the cake was washed with EA (60 mL×3). The filtrate was concentrated and the residue was diluted with EA (60 mL). The organic layer was washed with water (100 mL) and brine (100 mL), dried over anhydrous Na₂SO₄, filtered and concentrated under reduced pressure to give 6-chloro-4-methylpyridine-2,3-diamine (800 mg, yield 95%) as a yellow solid. LC-MS (ESI): mass calced for: C₆H&ClN₃, 157.04; m/z found, 158.2 [M+H]⁺.

### Step C: 5-chloro-7-methyl-3H-imidazo[4,5-b]pyridine

To a solution of 6-chloro-4-methylpyridine-2,3-diamine (1.22 g, 7.74 mmol, 1.0 eq) in Trimethyl orthoformate (TMOF) (30.0 mL) was added Formic Acid (0.75 mL) at room temperature. The reaction mixture was stirred at 90° C. for 2 h. After evaporation, the crude was purified by flash column chromatography on silica gel (EA, 100% v/v) to provide 5-chloro-7-methyl-3H-imidazo[4,5-b]pyridine (0.60 g, yield 46%) as a yellow solid. LC-MS (ESI): mass calced for: C₇H₆ClN₃, 167.03; m/z found, 168.1 [M+H]⁺.

### Step D: 3-(5-chloro-7-methyl-3H-imidazo[4,5-b]pyridin-3-yl) thietane 1,1-dioxide

To a solution of 5-chloro-7-methyl-3H-imidazo[4,5-b]pyridine (100 mg, 597 μmol, 1.0 eq) in DMF (6 mL) was added Sodium hydride (60% suspend in oil) (35.8 mg, 895 μmol, 15 eq) at 0° C. The mixture was stirred at 0° C. for 1 h. Then a solution of 3-bromothietane 1,1-dioxide (132 mg, 716 μmol, 1.2 eq) in DMF (2 mL) was dropwise added to above mixture at 0° C. The reaction mixture was stirred for 1 h at room temperature, quenched with ice-water (15 mL), and extracted with EtOAc (10 mL×3). The organic layers were washed with brine (10 mL×4), dried over anhydrous Na₂SO₄, filtered and concentrated in vacuum. The crude product was purified by Prep-TLC(EA, 100% v/v) to give 3-(5-chloro-7-methyl-3H-imidazo[4,5-b]pyridin-3-yl) thietane 1,1-dioxide (20.0 mg, yield 12%) as a yellow solid. LC-MS (ESI): mass calced for: C₁₀H₁₀ClN₃O₂S, 271.02; m/z found, 272.1 [M+H]⁺. ¹H NMR (400 MHz, DMSO-d₆) δ 8.67 (s, 1H), 7.30 (s, 1H), 5.53-5.46 (m, 1H), 4.89 (d, J=6.8 Hz, 4H), 2.58 (s, 3H).

### Step E: 3-(7-(bromomethyl)-5-chloro-3H-imidazo[4,5-b]pyridin-3-yl) thietane 1,1-dioxide

To a suspension of 3-(5-chloro-7-methyl-3H-imidazo[4,5-b]pyridin-3-yl) thietane 1,1-dioxide (52.0 mg, 191 μmol, 1.0 eq) and N-bromosuccinimide (37.5 mg, 211 μmol, 1.1 eq) in CCl₄ (8.00 mL) was added Benzoyl peroxide (6.95 mg, 28.7 μmol, 0.15 eq). The reaction mixture was stirred at 90° C. for 16 h. After cooled to room temperature, the mixture was diluted with DCM (50 mL), washed with saturated aqueous NaHCO₃ solution (50 mL×2) and brine (50 mL). The organic layers were dried over anhydrous Na₂SO₄, filtered and concentrated under reduced pressure. The crude product was purified by Prep-TLC (DCM/MeOH=15/1 v/v) to give 3-(7-(bromomethyl)-5-chloro-3H-imidazo[4,5-b]pyridin-3-yl) thietane 1,1-dioxide (5.00 mg, yield 7%) as a white solid. LC-MS (ESI): mass calced for: C₁₀H₉BrClN₃O₂S, 348.93; m/z found, 349.94 [M+H]⁺. ¹H NMR (400 MHZ, DMSO-d₆) δ 8.79 (s, 1H), 7.54 (s, 1H), 5.56-5.48 (m, 1H), 5.08 (s, 2H), 4.92 (d, J=6.8 Hz, 4H).

### Intermediate 82:4-(bromomethyl)-2-chloro-8-isopropoxy-1,5-naphthyridine

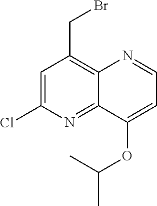 [SMILES: CC(C)Oc1ccnc2c(CBr)cc(Cl)nc12]

### Step A: 2-chloro-8-isopropoxy-4-methyl-1,5-naphthyridine

To a solution of 6-chloro-8-methyl-1,5-naphthyridin-4-ol (50.0 mg, 257 μmol, 1.0 eq) in DMF (2.00 mL) were added Cs₂CO₃ (251 mg, 771 μmol, 3.0 eq) and Isopropyl iodide (131 mg, 77.0 μL, 771 μmol, 3.0 eq). The mixture was stirred at 80° C. for 3 h. After cooled to room temperature, the reaction mixture was quenched with saturated aqueous NH₄Cl solution (5 mL) and extracted with EA (5 mL×3). The organic layer was washed with brine (5 mL×4), dried over MgSO₄, filtered and concentrated under reduced pressure. The crude product was purified by Prep-TLC(PE/EA=1/1 v/v) to obtain 2-chloro-8-isopropoxy-4-methyl-1,5-naphthyridine (60.0 mg, yield 98%) as a yellow oil. LC-MS (ESI): mass calced for: C₁₂H₁₃ClN₂O, 236.07; m/z found, 237.1 [M+H]⁺.

### Step B: 4-(bromomethyl)-2-chloro-8-isopropoxy-1,5-naphthyridine

To a solution of 2-chloro-8-isopropoxy-4-methyl-1,5-naphthyridine (60.0 mg, 253 μmol, 1.0 eq) in CCl₄ (4.00 mL) were added NBS (49.6 mg, 279 μmol, 1.1 eq) and Benzoyl peroxide (6.14 mg, 25.3 μmol, 0.1 eq). The mixture was stirred at 90° C. for 3 h. The reaction mixture was cooled to room temperature and concentrated under reduced pressure. The crude product was purified by Prep-TLC(PE/EA=2/1 v/v) to obtain 4-(bromomethyl)-2-chloro-8-isopropoxy-1,5-naphthyridine (35.0 mg, yield 43%) as a white solid. LC-MS (ESI): mass calced for: C₁₂H₁₂BrClN₂O, 315.60; m/z found, 316.6 [M+H]⁺.

### Intermediate 83:2-chloro-7-oxo-6,7-dihydro-5H-pyrrolo[3,4-b]pyridine-4-carbaldehyde

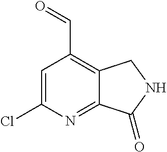 [SMILES: O=Cc1cc(Cl)nc2c1CNC2=O]

### Step A: 2-(methoxycarbonyl)-3-methylpyridine 1-oxide

To a stirred mixture of methyl 3-methylpicolinate (10.0 g, 66.2 mmol, 1.0 eq) in DCM (150 mL) was added m-CPBA (17.1 g, 99.2 mmol, 1.5 eq) at 0° C. The resulting mixture was stirred at 25° C. for 16 h. The reaction mixture was quenched with aqueous NaHCO₃ solution (200 mL) and extracted with EtOAc (200 mL×3). The combined organic layers were washed with brine (200 mL), dried over anhydrous Na₂SO₄, filtered and concentrated in vacuum. The residue was purified by flash column chromatography on silica gel (PE/EA=10/1 v/v) to give 2-(methoxycarbonyl)-3-methylpyridine 1-oxide (7.0 g, yield 63%) as a yellow oil. LC-MS (ESI): mass calced for: C₈H₉NO₃, 167.06; m/z found, 168.1 [M+H]⁺.

### Step B: 2-(methoxycarbonyl)-3-methyl-4-nitropyridine 1-oxide

To a stirred mixture of 2-(methoxycarbonyl)-3-methylpyridine 1-oxide (1.0 g, 5.98 mmol, 1.0 eq) in con. H₂SO₄ (15.0 mL) was added fuming Nitric acid (1.88 g, 1.53 mL, 29.9 mmol, 5.0 eq) at 0° C. The resulting mixture was stirred at 100° C. for 16 h. After cooled to room temperature, the reaction mixture was adjusted to pH 7-8 with diluted aqueous K₂CO₃ solution (1 N) and extracted with EtOAc (100 mL×3). The combined organic layers were washed with brine (50 mL) and dried over anhydrous Na₂SO₄, filtered and concentrated in vacuum. The residue was purified by flash column chromatography on silica gel (PE/EtOAc=1/1 v/v) to give 2-(methoxycarbonyl)-3-methyl-4-nitropyridine 1-oxide (220 mg, yield 17%) as a white solid. LC-MS (ESI): mass calced for: C₈H₈N₂O₅, 212.04; m/z found, 213.0 [M+H]⁺.

### Step C: 4-bromo-2-(methoxycarbonyl)-3-methylpyridine 1-oxide

To a stirred mixture of 2-(methoxycarbonyl)-3-methyl-4-nitropyridine 1-oxide (2.30 g, 10.8 mmol, 1.0 eq) in AcOH (25.0 mL) was added Acetyl bromide (6.66 g, 4.01 mL, 54.2 mmol, 5.0 eq) at 0° C. The resulting mixture was stirred at 80° C. for 1 h. After cooled to room temperature, the reaction mixture was adjusted to pH 7-8 with diluted aqueous NaHCO₃ solution (1 N) and extracted with EtOAc (100 mL×3). The combined organic layers were washed with brine (100 mL), dried over anhydrous Na₂SO₄, filtered and concentrated under reduced pressure. The residue was purified by flash column chromatography on silica gel (PE/EtOAc=1/1 v/v) to give 4-bromo-2-(methoxycarbonyl)-3-methylpyridine 1-oxide (1.20 g, yield 45%) as a white solid. LC-MS (ESI): mass calced for: C₈H₈BrNO3, 244.97; m/z found, 245.9 [M+H]⁺. ¹H NMR (400 MHZ, DMSO-d₆) δ 8.15 (d, J=6.8 Hz, 1H), 7.81 (d, J=6.8 Hz, 1H), 3.93 (s, 3H), 2.23 (s, 3H).

### Step D: methyl 4-bromo-6-chloro-3-methylpicolinate

To a stirred mixture of 4-bromo-2-(methoxycarbonyl)-3-methylpyridine 1-oxide (2.50 g, 10.2 mmol, 1.0 eq) in toluene (30.0 mL) was added POCl₃ (3.12 g, 1.89 mL, 20.3 mmol, 2.0 eq). The resulting mixture was stirred at 100° C. for 2 h. After cooled to room temperature, the reaction mixture was quenched with saturated aqueous NaHCO₃ solution (50 mL) and extracted with EtOAc (80 mL×3). The combined organic layers were washed with brine (80 mL), dried over anhydrous Na₂SO₄, filtered and concentrated in vacuum. The residue was purified by flash column chromatography on silica gel (PE/EtOAc=5/1 v/v) to obtain methyl 4-bromo-6-chloro-3-methylpicolinate (600 mg, yield 22%) as a white solid. LC-MS (ESI): mass calced for: C₈H₇BrClNO2, 262.93; m/z found, 263.9 [M+H]⁺. ¹H NMR (400 MHZ, DMSO-d₆) δ 8.58 (s, 1H), 3.92 (s, 3H), 2.59 (s, 3H).

### Step E: methyl 4-bromo-3-(bromomethyl)-6-chloropicolinate

To a stirred mixture of methyl 4-bromo-6-chloro-3-methylpicolinate (1.20 g, 4.54 mmol, 1.0 eq) in CCl₄ (15.0 mL) were added NBS (969 mg, 5.44 mmol, 1.2 eq) and Benzoyl peroxide (549 mg, 2.27 mmol, 0.5 eq). The resulting mixture was stirred under N₂ at 80° C. for 16 h. After cooled to room temperature, the reaction mixture was quenched with saturated aqueous Na₂S₂O₃ solution (80 mL) and extracted with EtOAc (80 mL×3). The combined organic layers were washed with brine (80 mL), dried over anhydrous Na₂SO₄, filtered and concentrated in vacuum. The residue was purified by flash column chromatography on silica gel (PE/EtOAc=1/1 v/v) to give methyl 4-bromo-3-(bromomethyl)-6-chloropicolinate (1.10 g, yield 70%) as a yellow solid. LC-MS (ESI): mass calced for: C₈H₆Br₂ClNO₂, 340.85; m/z found, 341.8 [M+H]⁺.

### Step F: 4-bromo-2-chloro-5,6-dihydro-7H-pyrrolo[3,4-b]pyridin-7-one

To a stirred mixture of methyl 4-bromo-3-(bromomethyl)-6-chloropicolinate (1.0 g, 2.91 mmol, 1.0 eq) in MeOH (9.0 mL) was added Ammonia (3.0 mL, 77.5 mmol, 27 eq). The resulting mixture was stirred at 25° C. for 20 min. The reaction mixture was diluted with H₂O (50 mL) and extracted with EtOAc (50 mL×3). The combined organic layers were washed with brine (50 mL×3), dried over anhydrous Na₂SO₄ and concentrated. The residue was purified by flash column chromatography on silica gel (PE/EtOAc=1/1 v/v) to give 4-bromo-2-chloro-5,6-dihydro-7H-pyrrolo[3,4-b]pyridin-7-one (250 mg, yield 35%) as a white solid. LC-MS (ESI): mass calced for: C₇H₄BrClN₂O, 245.92; m/z found, 246.9 [M+H]⁺.

### Step G: 2-chloro-4-vinyl-5,6-dihydro-7H-pyrrolo[3,4-b]pyridin-7-one

To a stirred solution of Potassium vinyltrifluoroborate (130 mg, 970 μmol, 1.2 eq) and 4-bromo-2-chloro-5,6-dihydro-7H-pyrrolo[3,4-b]pyridin-7-one (200 mg, 808 μmol, 1.0 eq) in 1,4-Dioxane (10.0 mL) and H₂O (1.0 mL) were added Pd(Ph₃P)₄ (93.4 mg, 80.8 μmol, 0.1 eq) and Na₂CO₃ (257 mg, 2.42 mmol, 3.0 eq). The reaction mixture was stirred under N₂ at 95° C. for 2 h. After cooled to room temperature, the mixture was diluted with H₂O (20 mL) and extracted with EtOAc (50 mL×3). The combined organic layers were washed with brine (50 mL), dried over anhydrous Na₂SO₄ and concentrated under reduced pressure. The residue was purified by flash column chromatography on silica gel (PE/EtOAc=3/1 v/v) to give 2-chloro-4-vinyl-5,6-dihydro-7H-pyrrolo[3,4-b]pyridin-7-one (120 mg, yield 76%) as a brown solid. LC-MS (ESI): mass calced for: C₉H₇ClN₂O, 194.02; m/z found, 195.0 [M+H]⁺.

### Step H: 2-chloro-7-oxo-6,7-dihydro-5H-pyrrolo[3,4-b]pyridine-4-carbaldehyde

Ozone was bubbled into a solution of 2-chloro-4-vinyl-5,6-dihydro-7H-pyrrolo[3,4-b]pyridin-7-one (110 mg, 565 μmol, 1.0 eq) in DCM (10.0 mL) at −78° C. for 1 h. On completion, excess Ozone was purged from the reaction with nitrogen and dimethylsulfide (1 mL) was added. The reaction mixture was stirred at room temperature for 0.5 h. After evaporation, the residue was purified by flash chromatography on silica gel (PE/EA=1/1 v/v) to give 2-chloro-7-oxo-6,7-dihydro-5H-pyrrolo[3,4-b]pyridine-4-carbaldehyde (60.0 mg, yield 54%) as a light yellow solid. LC-MS (ESI): mass calced for: C₈H₅ClN₂O₂, 196.00; m/z found, 197.0 [M+H]⁺.

### Intermediate 84:5-chloro-1-(oxetan-3-yl)-7-(pyrrolidin-1-ylmethyl)-1H-pyrazolo[4,3-b]pyridin-3-amine

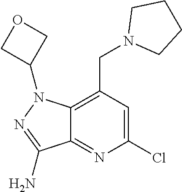 [SMILES: Nc1nn(C2COC2)c2c(CN3CCCC3)cc(Cl)nc12]

### Step A: 2-(7-bromo-5-chloro-1-(oxetan-3-yl)-1H-pyrazolo[4,3-b]pyridin-3-yl) isoindoline-1,3-dione

To a solution of 2-(7-bromo-5-chloro-1H-pyrazolo[4,3-b]pyridin-3-yl) isoindoline-1,3-dione (2.4 g, 6.4 mmol, 1.0 eq), PPh₃ (3.3 g, 12.7 mmol, 2.0 eq), and oxetan-3-ol (94.0 mg, 12.7 mmol, 2.0 eq) in THF (35.0 mL) was added DIAD (2.5 mL, 12.7 mmol, 2.0 eq) at 0° C. and the mixture was stirred under N₂ at 50° C. for 12 h. The reaction mixture was cooled to room temperature, quenched with H₂O (20 mL), and extracted with EA (30 mL×3). The organic layer was washed with brine (30 mL×4), dried over anhydrous Na₂SO₄, filtered and concentrated under reduced pressure. The residue was purified by flash column chromatography on silica gel (PE/EA=5/1 v/v) to give 2-(7-bromo-5-chloro-1-(oxetan-3-yl)-1H-pyrazolo[4,3-b]pyridin-3-yl) isoindoline-1,3-dione (1.5 g, yield 54%) as a yellow solid. LC-MS (ESI): mass calcd. for C₁₇H₁₀BrClN₄O₃, 432.0; m/z found, 433.0 [M+H]⁺.

### Step B: 2-(5-chloro-1-(oxetan-3-yl)-7-vinyl-1H-pyrazolo[4,3-b]pyridin-3-yl) isoindoline-1,3-dione

To a solution of 2-(7-bromo-5-chloro-1-(oxetan-3-yl)-1H-pyrazolo[4,3-b]pyridin-3-yl) isoindoline-1,3-dione (600 mg, 1.4 mmol, 1.0 eq) in DMF (25.0 mL) were added Pd (PPh₃)₄ (160 mg, 138 μmol, 0.1 eq) and tributyl (vinyl) stannane (658 mg, 2.1 mmol, 1.5 eq) at 25° C. and the mixture was stirred under N₂ at 70° C. for 3 h. The reaction mixture was cooled to room temperature, quenched with H₂O (20 mL), and extracted with EA (30 mL×3). The organic layer was washed with brine (30 mL×4), dried over anhydrous Na₂SO₄, filtered and concentrated under reduced pressure. The residue was purified by flash column chromatography on silica gel (PE/EA=5/1 v/v) to give 2-(5-chloro-1-(oxetan-3-yl)-7-vinyl-1H-pyrazolo[4,3-b]pyridin-3-yl) isoindoline-1,3-dione (100 mg, yield 19%) as a yellow solid. LC-MS (ESI): mass calcd. for C₁₉H₁₃ClN₄O₃, 380.1; m/z found, 381.0 [M+H]⁺. ¹H NMR (400 MHz, DMSO-d₆) δ .09-8.07 (m, 2H), 8.04-8.01 (m, 2H), 7.63 (s, 1H), 7.30 (dd, J=17.2, 11.2 Hz, 1H), 6.21-6.12 (m, 2H), 5.84 (d, J=11.8 Hz, 1H), 5.04 (d, J=6.6 Hz, 4H).

### Step C: 2-(5-chloro-7-(1,2-dihydroxyethyl)-1-(oxetan-3-yl)-1H-pyrazolo[4,3-b]pyridin-3-yl) isoindoline-1,3-dione

To a solution of 2-(5-chloro-1-(oxetan-3-yl)-7-vinyl-1H-pyrazolo[4,3-b]pyridin-3-yl) isoindoline-1,3-dione (332 mg, 872 μmol, 1.0 eq) in acetone (10.0 mL) and H₂O (5.0 mL) were added NMO (204 mg, 1.7 mmol, 2 eq) and K₂OsO₄·2H₂O (64.2 mg, 174 μmol, 0.2 eq) at 25° C. and the reaction mixture was stirred at 25° C. for 1 h. The reaction mixture was quenched with H₂O (20 mL) and extracted with EA (30 mL×3). The organic layer was washed with brine (30 mL×4), dried over anhydrous Na₂SO₄, and filtered. The filtrate was concentrated under reduced pressure to afford 2-(5-chloro-7-(1,2-dihydroxyethyl)-1-(oxetan-3-yl)-1H-pyrazolo[4,3-b]pyridin-3-yl) isoindoline-1,3-dione (270 mg, yield 75%) as a yellow solid. LC-MS (ESI): mass calcd. for C₁₉H₁₅ClN₄O₅, 414.1; m/z found, 415.0 [M+H]⁺.

### Step D: 5-chloro-3-(1,3-dioxoisoindolin-2-yl)-1-(oxetan-3-yl)-1H-pyrazolo[4,3-b]pyridine-7-carbaldehyde

To a solution of 2-(5-chloro-7-(1,2-dihydroxyethyl)-1-(oxetan-3-yl)-1H-pyrazolo[4,3-b]pyridin-3-yl) isoindoline-1,3-dione (270 mg, 651 μmol, 1.0 eq) in THF (10.0 mL) and H₂O (5.0 mL) was added NaIO₄ (278 mg, 1.3 mmol, 2.0 eq) at 25° C. and the reaction mixture was stirred at 25° C. for 1 h. The reaction mixture was quenched with H₂O (10 mL), and extracted with EA (10 mL×3). The organic layer was washed with brine (20 mL×2), dried over anhydrous Na₂SO₄, filtered and concentrated under reduced pressure. The residue was purified by Prep-TLC(PE/EA=1/1 v/v) to afford 5-chloro-3-(1,3-dioxoisoindolin-2-yl)-1-(oxetan-3-yl)-1H-pyrazolo[4,3-b]pyridine-7-carbaldehyde (37.0 mg, yield 15%) as a yellow solid. LC-MS (ESI): mass calcd. for C₁₈H₁₁ClN₄O₄, 382.1; m/z found, 383.0 [M+H]⁺.

### Step E: 2-(5-chloro-1-(oxetan-3-yl)-7-(pyrrolidin-1-ylmethyl)-1H-pyrazolo[4,3-b]pyridin-3-yl) isoindoline-1,3-dione

To a solution of 5-chloro-3-(1,3-dioxoisoindolin-2-yl)-1-(oxetan-3-yl)-1H-pyrazolo[4,3-b]pyridine-7-carbaldehyde (100 mg, 261 μmol, 1.0 eq) and pyrrolidine (55.7 mg, 784 μmol, 3.0 eq) in DCM (10.0 mL) was added AcOH (15.7 mg, 261 μmol, 1.0 eq) at 25° C., the mixture was stirred at 25° C. for 12 h. NaBH(OAc) 3 (166 mg, 784 μmol, 3.0 eq) was added to above mixture and the reaction mixture was stirred at room temperature for 2.5 h. The reaction mixture was quenched with H₂O (10 mL) and extracted with DCM (10 mL×3). The organic layer was washed with brine (10 mL), dried over anhydrous Na₂SO₄, filtered and concentrated under reduced pressure. The residue was purified by Prep-TLC(EA/PE=1/3 v/v) to afford 2-(5-chloro-1-(oxetan-3-yl)-7-(pyrrolidin-1-ylmethyl)-1H-pyrazolo[4,3-b]pyridin-3-yl) isoindoline-1,3-dione (50.0 mg, yield 44%) as a yellow solid. LC-MS (ESI): mass calcd. for C₂₂H₂OClN₅O₃, 437.1; m/z found, 438.1 [M+H]⁺.

### Step F: 5-chloro-1-(oxetan-3-yl)-7-(pyrrolidin-1-ylmethyl)-1H-pyrazolo[4,3-b]pyridin-3-amine

To a solution of 2-(5-chloro-1-(oxetan-3-yl)-7-(pyrrolidin-1-ylmethyl)-1H-pyrazolo[4,3-b]pyridin-3-yl) isoindoline-1,3-dione (50.0 mg, 114 μmol, 1.0 eq) in EtOH (3.0 mL) was added Hydrazine hydrate (80% W.t in water) (10.3 mg, 343 μmol, 3.0 eq) at 25° C. and the reaction mixture was stirred at 25° C. for 0.5 h. The reaction mixture was quenched with H₂O (10 mL) and extracted with DCM (10 mL×3). The organic layer was washed with brine (10 mL), dried over anhydrous Na₂SO₄, filtered and concentrated under reduced pressure. The residue was purified by Prep-TLC(DCM/MeOH=10/1 v/v) to afford 5-chloro-1-(oxetan-3-yl)-7-(pyrrolidin-1-ylmethyl)-1H-pyrazolo[4,3-b]pyridin-3-amine (22.0 mg, yield 63%) as a yellow solid. LC-MS (ESI): mass calcd. for C₁₄H₁₈ClN₅O,307.1; m/z found, 308.1 [M+H]⁺.

### Intermediate 85:5-chloro-3-methoxy-1-(tetrahydro-2H-pyran-2-yl)-1H-pyrazolo[4,3-b]pyridine-7-carbaldehyde

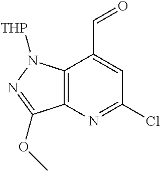

### Step A: methyl 6-chloro-3-fluoropicolinate

To a solution of 6-chloro-3-fluoropicolinic acid (5.00 g, 28.5 mmol, 1.0 eq) in MeOH (50.0 mL) was added dropwise con. H₂SO₄ (5.00 mL) at 0° C. The mixture was stirred at 70° C. for 16 h. After evaporation, the residue was quenched with saturated aqueous NaHCO₃ solution, diluted with water (200 mL), and extracted with EA (200 mL×3). The organic layer was washed with brine (100 mL), dried over MgSO₄, filtered and concentrated under reduced pressure. The residue was purified by flash column chromatography on silica gel (ethyl acetate in petroleum ether, 20% v/v) to give methyl 6-chloro-3-fluoropicolinate (4.50 g, yield 75%) as a yellow solid. LC-MS (ESI): mass calced for: C₇H₅CIFNO₂, 189.1; m/z found, 190.1 [M+H]⁺.

### Step B: 5-chloro-1,2-dihydro-3H-pyrazolo[4,3-b]pyridin-3-one

To a stirred mixture of methyl 6-chloro-3-fluoropicolinate (1.50 g, 7.91 mmol, 1.0 eq) in EtOH (10.0 mL) was added N₂H₄H₂O (317 mg, 6.33 mmol, 0.8 eq). The resulting mixture was stirred in microwave at 110° C. for 2 h. After cooled to room temperature, the mixture was filtered and the cake was dried to gave crude product 5-chloro-1,2-dihydro-3H-pyrazolo[4,3-b]pyridin-3-one (1.00 g, yield 67%) as a yellow solid. LC-MS (ESI): mass calced for: C₆H₄ClN₃O, 169.1; m/z found, 170.1 [M+H]⁺.

### Step C: 5-chloro-1-(tetrahydro-2H-pyran-2-yl)-1,2-dihydro-3H-pyrazolo[4,3-b]pyridin-3-one

To a solution of 5-chloro-1,2-dihydro-3H-pyrazolo[4,3-b]pyridin-3-one (1.50 g, 8.85 mmol, 1.0 eq) and 3,4-dihydro-2H-pyran (1.49 g, 17.7 mmol, 2.0 eq) in DCM (30.0 mL) was added 4-methylbenzenesulfonic acid hydrate (168 mg, 885 μmol, 0.1 eq). The mixture was stirring at room temperature for 16 h. The mixture was poured into water (5 mL) and extracted with DCM (15 mL×3). The organic layer was washed with saturated aqueous NaHCO₃ solution (30 mL×2) and brine (30 mL), dried over MgSO₄, filtered and concentrated under reduced pressure. The residue was purified by flash column chromatography on silica gel (ethyl acetate in petroleum ether, 50% v/v) to give 5-chloro-1-(tetrahydro-2H-pyran-2-yl)-1,2-dihydro-3H-pyrazolo[4,3-b]pyridin-3-one (350 mg, yield 14%) as a yellow solid. LC-MS (ESI): mass calced for: C₁₁H₁₂ClN₃O₂, 253.1; m/z found, 254.1 [M+H]⁺.

### Step D: 5-chloro-3-methoxy-1-(tetrahydro-2H-pyran-2-yl)-1H-pyrazolo[4,3-b]pyridine

To a solution of 5-chloro-1-(tetrahydro-2H-pyran-2-yl)-1,2-dihydro-3H-pyrazolo[4,3-b]pyridin-3-one (140 mg, 552 μmol, 1.0 eq) in DMF (3.00 mL) were added sodium hydride (60% suspend in oil) (15.9 mg, 662 μmol, 1.2 eq) under N₂ at 0° C. and the mixture was stirred for 0.5 h. Then iodomethane (117 mg, 828 μmol, 1.5 eq) was added to above mixture and the mixture was stirred at 0° C. for 1.5 h. The mixture was quenched with saturated aqueous NH₄Cl solution (2 mL), diluted with water (5 mL), and extracted with EtOAc (10 mL×3). The organic layer was dried over MgSO₄, filtered and concentrated under reduced pressure. The residue was purified by Prep-TLC(PE/EA=3/2 v/v) to obtain 5-chloro-3-methoxy-1-(tetrahydro-2H-pyran-2-yl)-1H-pyrazolo[4,3-b]pyridine (70.0 mg, yield 43%) as a yellow oil. LC-MS (ESI): mass calced for: C₁₂H₁₄ClN₃O₂, 267.1; m/z found, 268.1 [M+H]⁺. ¹H NMR (400 MHZ, DMSO-d₆) δ 8.20 (d, J=8.8 Hz, 1H), 7.51 (d, J=8.8 Hz, 1H), 5.74-5.71 (m, 1H), 4.06 (s, 3H), 3.93-3.84 (m, 1H), 3.74-3.66 (m, 1H), 2.32-2.27 (m, 1H), 1.96-1.92 (m, 1H), 1.85-1.63 (m, 2H), 1.62-1.49 (m, 2H).

### Step E: 5-chloro-3-methoxy-1-(tetrahydro-2H-pyran-2-yl)-1H-pyrazolo[4,3-b]pyridine-7-carbaldehyde

To a solution of 5-chloro-3-methoxy-1-(tetrahydro-2H-pyran-2-yl)-1H-pyrazolo[4,3-b]pyridine (70.0 mg, 261 μmol, 1.0 eq) in THF (3.00 mL) was added dropwise n-Butyllithium (1.6 M in hexane) (245 μL, 392 μmol, 1.5 eq) under N₂ at −78° C. and the mixture was stirred at −78° C. for 0.5 h. Then DMF (28.7 mg, 30.4 μL, 392 μmol, 1.5 eq) was added to above mixture and the mixture was stirred at −78° C. for anther 1.5 h. The reaction mixture was warmed to room temperature, quenched with saturated aqueous NH₄Cl solution (0.5 mL), diluted with water (5 mL), and extracted with EtOAc (10 mL×3). The organic layer was dried over MgSO₄, filtered and concentrated under reduced pressure. The residue was purified by Prep-TLC(PE/EA=3/2 v/v) to obtain 5-chloro-3-methoxy-1-(tetrahydro-2H-pyran-2-yl)-1H-pyrazolo[4,3-b]pyridine-7-carbaldehyde (60.0 mg, yield 70%) as a yellow oil. LC-MS (ESI): mass calced for: C₁₃H₁₃ClN₃O₃, 295.1; m/z found, 296.1 [M+H]⁺.

### Intermediate 86:5-chloro-3-isopropoxy-1-(tetrahydro-2H-pyran-2-yl)-1H-pyrazolo[4,3-b]pyridine-7-carbaldehyde

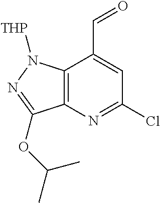

### Step A: 5-chloro-3-isopropoxy-1-(tetrahydro-2H-pyran-2-yl)-1H-pyrazolo[4,3-b]pyridine

To a solution of 5-chloro-1-(tetrahydro-2H-pyran-2-yl)-1H-pyrazolo[4,3-b]pyridin-3-ol (150 mg, 591 μmol, 1.0 eq) in DMF (3.00 mL) were added NaH (60% suspend in oil) (17.0 mg, 710 μmol, 1.2 eq) under N₂ at 0° C. and the mixture was stirred for 0.5 h. Then Isopropyl iodide (151 mg, 88.6 μL, 887 μmol, 1.5 eq) was added to above mixture and the mixture was stirred at 0° C. for 1.5 h. The mixture was quenched with saturated aqueous NH₄Cl solution (2 mL), diluted with water (5 mL), and extracted with EtOAc (10 mL×3). The organic layer was dried over MgSO₄, filtered and concentrated under reduced pressure. The residue was purified by Prep-TLC(PE/EA=3/2 v/v) to obtain 5-chloro-3-isopropoxy-1-(tetrahydro-2H-pyran-2-yl)-1H-pyrazolo[4,3-b]pyridine (60.0 mg, yield 31%) as a yellow oil. LC-MS (ESI): mass calced for: C₁₄H₁₈ClN₃O₂, 295.1; m/z found, 296.2 [M+H]⁺.

### Step B: 5-chloro-3-isopropoxy-1-(tetrahydro-2H-pyran-2-yl)-1H-pyrazolo[4,3-b]pyridine-7-carbaldehyde

To a solution of 5-chloro-3-isopropoxy-1-(tetrahydro-2H-pyran-2-yl)-1H-pyrazolo[4,3-b]pyridine (60.0 mg, 203 μmol, 1.0 eq) in THF (3.00 mL) was added butyllithium (1.6 Min n-hexane) (0.19 mL, 304 μmol, 1.5 eq) under N₂ at −78° C. and the mixture was stirred at −78° C. for 0.5 h. Then anhydrous N,N-dimethylformamide (22.2 mg, 304 μmol, 1.5 eq) was added to above mixture and the mixture was stirred at −78° C. for anther 1.5 h. The reaction mixture was warmed to room temperature, quenched with saturated aqueous NH₄Cl solution (0.5 mL), diluted with water (5 mL), and extracted with EtOAc (10 mL×3). The organic layer was dried over MgSO₄, filtered and concentrated under reduced pressure. The residue was purified by Prep-TLC(PE/EA=3/2 v/v) to obtain 5-chloro-3-isopropoxy-1-(tetrahydro-2H-pyran-2-yl)-1H-pyrazolo[4,3-b]pyridine-7-carbaldehyde (50.0 mg, yield 69%) as a yellow oil. LC-MS (ESI): mass calced for: C₁₅H₁₈ClN₃O₃, 323.1; m/z found, 324.2 [M+H]⁺.

### Intermediate 87:4-(bromomethyl)-2-chloro-8-cyclobutoxy-1,5-naphthyridine

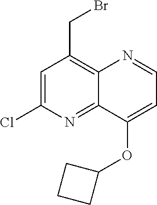 [SMILES: Clc1cc(CBr)c2nccc(OC3CCC3)c2n1]

### Step A: 2-chloro-8-cyclobutoxy-4-methyl-1,5-naphthyridine

To a solution of 6-chloro-8-methyl-1,5-naphthyridin-4-ol (100 mg, 514 μmol, 1.0 eq) in DMF (3.00 mL) were added Cs₂CO₃ (502 mg, 1.54 mmol, 3.0 eq) and bromocyclobutane (208 mg, 1.54 mmol, 3.0 eq). The mixture was stirred at 80° C. for 3 h. After cooled to room temperature, the reaction mixture was quenched with saturated aqueous NH₄Cl solution (10 mL) and extracted with EA (10 mL×3). The organic layer was washed with brine (10 mL×4), dried over MgSO₄, filtered and concentrated under reduced pressure. The crude product was purified by Prep-TLC(PE/EA=1/1 v/v) to obtain 2-chloro-8-cyclobutoxy-4-methyl-1,5-naphthyridine (120 mg, yield 94%) as a yellow oily. LC-MS (ESI): mass calced for: C₁₃H₁₃ClN₂O, 248.71; m/z found, 249.2 [M+H]⁺.

### Step B: 4-(bromomethyl)-2-chloro-8-cyclobutoxy-1,5-naphthyridine

To a solution of 2-chloro-8-cyclobutoxy-4-methyl-1,5-naphthyridine (120 mg, 483 μmol, 1.0 eq) in CCl₄ (7.00 mL) were added NBS (95 mg, 532 μmol, 1.1 eq) and Benzoyl peroxide (11.7 mg, 48.3 μmol, 0.1 eq). The mixture was stirred at 90° C. for 3 h. After cooled to room temperature, the reaction mixture was quenched with water (10 mL) and extracted with DCM (10 mL×3). The organic layer was washed with brine (10 mL), dried over MgSO₄, filtered and concentrated under reduced pressure. The crude product was purified by Prep-TLC (PE/EA=2/1 v/v) to obtain 4-(bromomethyl)-2-chloro-8-cyclobutoxy-1,5-naphthyridine (80.0 mg, yield 50%) as a white solid. LC-MS (ESI): mass calced for: C₁₃H₁₂BrClN₂O, 327.61; m/z found, 328.2 [M+H]⁺. ¹H NMR (400 MHZ, DMSO-d₆) δ 8.85 (d, J=5.2 Hz, 1H), 8.01 (s, 1H), 7.20 (d, J=5.2 Hz, 1H), 5.15 (s, 2H), 5.05-4.97 (m, 1H), 2.61-2.55 (m, 2H), 2.27-2.19 (m, 2H), 1.93-1.84 (m, 1H), 1.76-1.69 (m, 1H).

### Intermediate 88:6-chloro-3-methyl-1-(oxetan-3-yl)-1H-pyrrolo[2,3-b]pyridine-4-carbaldehyde

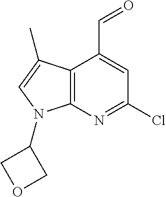 [SMILES: Cc1cn(C2COC2)c2nc(Cl)cc(C=O)c12]

### Step A: methyl 6-chloro-3-iodo-1H-pyrrolo[2,3-b]pyridine-4-carboxylate

To a solution of methyl 6-chloro-1H-pyrrolo[2,3-b]pyridine-4-carboxylate (5.0 g, 23.7 mmol, 1.0 eq) in DMF (66.0 mL) was added NIS (16.0 g, 71.2 mmol, 3.0 eq) at room temperature and the reaction mixture was stirred under N₂ at room temperature for 16 h. The reaction mixture was quenched with H₂O (200 mL) and extracted with EA (200 mL×3). The organic layer was washed with saturated aqueous Na₂S₂O₃ solution (200 mL×2) and brine (200 mL×3), dried over Na₂SO₄, filtered and concentrated under reduced pressure. The residue was purified by flash column chromatography on silica gel (PE/EA=2/1 v/v) to give methyl 6-chloro-3-iodo-1H-pyrrolo[2,3-b]pyridine-4-carboxylate (5.5 g, yield 69%) as a yellow solid. LC-MS (ESI): mass calced for: C₉H₆ClIN202, 335.9; m/z found, 336.9 [M+H]⁺.

### Step B: methyl 6-chloro-3-iodo-1-(oxetan-3-yl)-1H-pyrrolo[2,3-b]pyridine-4-carboxylate

To a solution of methyl 6-chloro-3-iodo-1H-pyrrolo[2,3-b]pyridine-4-carboxylate (1.4 g, 4.2 mmol, 1.0 eq) in DMF (15.0 mL) were added Cs₂CO₃ (2.7 g, 8.3 mmol, 2.0 eq) and 3-iodooxetane (1.2 g, 6.2 mmol, 1.5 eq) at 25° C. The reaction mixture was stirred under N₂ at 80° C. for 4 h. After cooled to room temperature, the reaction mixture was filtered and the organic layer was quenched with H₂O (40 mL) and extracted with EA (40 mL×3). The organic layer was washed with brine (30 mL×4), dried over Na₂SO₄, filtered and concentrated under reduced pressure. The residue was purified by flash column chromatography on silica gel (PE/EA=2/1 v/v) to give methyl 6-chloro-3-iodo-1-(oxetan-3-yl)-1H-pyrrolo[2,3-b]pyridine-4-carboxylate (815 mg, yield 50%) as a yellow solid. LC-MS (ESI): mass calced for: C₁₂H₁₀ClIN203, 391.9; m/z found, 393.0 [M+H]⁺. ¹H NMR (400 MHZ, DMSO-d₆) δ 8.42 (s, 1H), 7.46 (s, 1H), 5.95-5.88 (m, 1H), 4.99 (d, J=1.6 Hz, 2H), 4.97 (d, J=1.6 Hz, 2H), 3.97 (s, 3H).

### Step C: methyl 6-chloro-3-methyl-1-(oxetan-3-yl)-1H-pyrrolo[2,3-b]pyridine-4-carboxylate

To a solution of methyl 6-chloro-3-iodo-1-(oxetan-3-yl)-1H-pyrrolo[2,3-b]pyridine-4-carboxylate (900 mg, 2.0 mmol, 1.0 eq) and 2,4,6-Trimethyl-1,3,5,2,4,6-trioxatriborinane (50% W.t in THF) (644 μL, 2.0 mmol, 1.0 eq) in 1,4-Dioxane (5.0 mL) and H₂O (0.5 mL) were added Pd(dppf)Cl₂ (373 mg, 0.5 mmol, 0.3 eq) and K₂CO₃ (938 mg, 6.8 mmol, 3.0 eq) at 25° C. and the reaction mixture was stirred under N₂ at 110° C. for 1 h. After cooled to room temperature, the reaction mixture was quenched with H₂O (20 mL) and extracted with EA (20 mL×3). The organic layer was washed with brine (20 mL×3), dried over Na₂SO₄, filtered and concentrated under reduced pressure. The residue was purified by flash column chromatography on silica gel (PE/EA=2/1 v/v) to give methyl 6-chloro-3-methyl-1-(oxetan-3-yl)-1H-pyrrolo[2,3-b]pyridine-4-carboxylate (100 mg, yield 14%) as a yellow solid. LC-MS (ESI): mass calced for: C₁₃H₁₃ClN₂O₃, 280.1; m/z found, 281.1 [M+H]⁺. ¹H NMR (400 MHZ, DMSO-d₆) δ 8.03 (d, J=0.8 Hz, 1H), 7.44 (s, 1H), 5.95-5.86 (m, 1H), 5.01 (t, J=7.2 Hz, 2H), 4.91 (t, J=7.2 Hz, 2H), 3.94 (s, 3H), 2.33 (s, 3H).

### Step D: (6-chloro-3-methyl-1-(oxetan-3-yl)-1H-pyrrolo[2,3-b]pyridin-4-yl) methanol

To a solution of methyl 6-chloro-3-methyl-1-(oxetan-3-yl)-1H-pyrrolo[2,3-b]pyridine-4-carboxylate (175 mg, 623 μmol, 1.0 eq) in THF (5.0 mL) was added LiAlH₄ (47.3 mg, 1.3 mmol, 2.0 eq) at 0° C. and the reaction mixture was stirred at 0° C. for 15 min. The reaction mixture was quenched with water (10 mL) and extracted with EA (10 mL×3). The organic layer was washed with brine (20 mL), dried over Na₂SO₄, filtered and concentrated under reduced pressure. The residue was purified by flash column chromatography on silica gel (PE/EA=2/1 v/v) to give (6-chloro-3-methyl-1-(oxetan-3-yl)-1H-pyrrolo[2,3-b]pyridin-4-yl) methanol (138 mg, yield 88%) as a yellow solid. LC-MS (ESI): mass calced for: C₁₂H₁₃ClN₂O₂, 252.1; m/z found, 253.1 [M+H]⁺.

### Step E: 6-chloro-3-methyl-1-(oxetan-3-yl)-1H-pyrrolo[2,3-b]pyridine-4-carbaldehyde

To a solution of (6-chloro-3-methyl-1-(oxetan-3-yl)-1H-pyrrolo[2,3-b]pyridin-4-yl) methanol (138 mg, 546 μmol, 1.0 eq) in DMSO (5.0 mL) was added IBX (Purity 45% W.t) (459 mg, 1.6 mmol, 3.0 eq) at 25° C. and the reaction mixture was stirred at 25° C. for 15 min. The reaction mixture was quenched with saturated aqueous Na₂S₂O₃ solution (10 mL) and saturated aqueous NaHCO₃ solution (10 mL), and extracted with EA (20 mL×3). The organic layer was washed with brine (10 mL×3), dried over Na₂SO₄, filtered and concentrated under reduced pressure. The residue was purified by Prep-TLC(PE/EA=5/1 v/v) to afford 6-chloro-3-methyl-1-(oxetan-3-yl)-1H-pyrrolo[2,3-b]pyridine-4-carbaldehyde (75.0 mg, yield 55%) as a yellow solid. LC-MS (ESI): mass calced for: C₁₂H₁₁ClN₂O₂, 250.1; m/z found, 251.1 [M+H]⁺.

### Intermediate 89:8-cyclobutyl-4-(pyrrolidin-1-ylmethyl) quinolin-2-yl trifluoro methanesulfonate

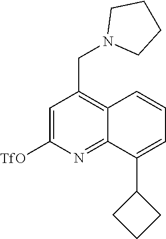 [SMILES: O=S(=O)(Oc1cc(CN2CCCC2)c2cccc(C3CCC3)c2n1)C(F)(F)F]

### Step A: N-(2-bromophenyl)-3-oxobutanamide

A mixture of 2-Bromobenzenamine (10.00 g, 58.13 mmol, 1.0 eq) in ethyl 3-oxobutanoate (14.72 mL, 116.3 mmol, 2.0 eq) was stirred at 180° C. for 2 h. After evaporation, the crude material was purified by flash column chromatography on silica gel (methanol in dichloromethane, from 0% to 5% v/v) to afford N-(2-bromophenyl)-3-oxobutanamide (6.50 g, yield 44%) as a white solid. LC-MS (ESI): mass calced for: C₁₀H₁₀BrNO₂, 254.99; m/z found, 256.2 [M+H]⁺. ¹H NMR (400 MHZ, DMSO-d₆) δ 9.73 (s, 1H), 7.72 (d, J=8.0 Hz, 1H), 7.69-7.63 (m, 1H), 7.40-7.35 (m, 1H), 7.13 (t, J=7.2 Hz, 1H), 3.65 (s, 2H), 2.23 (s, 3H).

### Step B: 8-bromo-4-methylquinolin-2-ol

N-(2-bromophenyl)-3-oxobutanamide (6.50 g, 25.4 mmol, 1.0 eq) was dissolved in con. sulfuric acid (10 mL) and the mixture was heated to 95° C. for 1 h. The crude mixture was cooled to room temperature, poured into ice-water (30 mL), and extracted with ethyl acetate (50 mL×3). The combined organic layers were washed with brine (50 mL×3), dried over anhydrous Na₂SO₄, filtered and concentrated under reduced pressure to afford 8-bromo-4-methylquinolin-2-ol (850 mg, yield 14%) as a white solid. LC-MS (ESI): mass calced for: C₁₀H₈BrNO, 236.98; m/z found, 238.1 [M+H]⁺. ¹H NMR (400 MHZ, DMSO-d₆) δ 10.23 (s, 1H), 7.83 (d, J=8.0 Hz, 1H), 7.76 (d, J=8.0 Hz, 1H), 7.17 (t, J=7.2 Hz, 1H), 6.51 (s, 1H), 2.45 (s, 3H).

### Step C: 8-bromo-4-(bromomethyl) quinolin-2-ol

To a stirred mixture of 8-bromo-4-methylquinolin-2-ol (1.00 g, 4.20 mmol, 1.0 eq) in CCl₄ (20.0 mL) were added AIBN (138 mg, 840 μmol, 0.2 eq) and NBS (897 mg, 5.04 mmol, 1.2 eq). The resulting mixture was stirred under N₂ at 90° C. for 3 h. After cooled to room temperature, the reaction mixture was quenched with saturated aqueous Na₂S₂O₃ (80 mL) and extracted with EtOAc (80 mL×3). The combined organic layers were washed with brine (100 mL×3), dried over anhydrous Na₂SO₄, filtered and concentrated under reduced pressure. The residue was purified by flash column chromatography on silica gel (PE/EtOAc=1/1 v/v) to give 8-bromo-4-(bromomethyl) quinolin-2-ol (610 mg, yield 46%) as a white solid. LC-MS (ESI): mass calced for: C₁₀H₇Br₂NO, 314.89; m/z found, 316.3 [M+H]⁺.

### Step D: 8-bromo-4-(pyrrolidin-1-ylmethyl) quinolin-2-ol

To a stirred mixture of 8-bromo-5-(bromomethyl) quinolin-2-ol (430 mg, 1.36 mmol, 1.0 eq) in DCM (5.00 mL) was added DIPEA (263 mg, 354 μL, 2.03 mmol, 1.5 eq). The resulting mixture was stirred at 25° C. for 10 min. Then pyrrolidine (116 mg, 134 μL, 1.63 mmol, 1.2 eq) was added to above mixture and the resulting mixture was stirred at 25° C. for 15 min. The reaction mixture was quenched with saturated aqueous NaHCO₃ solution (40 mL) and extracted with EtOAc (40 mL×3). The combined organic layers were washed with brine (40 mL), dried over anhydrous Na₂SO₄, filtered and concentrated under reduced pressure. The residue was purified by flash column chromatography on silica gel (DCM/MeOH=10/1 v/v) to give 8-bromo-5-(pyrrolidin-1-ylmethyl) quinolin-2-ol (623 mg, yield 100%, 70% Purity) as yellow oil. LC-MS (ESI): mass calced for: C₁₄H₁₅BrN₂O, 306.04; m/z found, 307.1 [M+H]⁺.

### Step E: 8-cyclobutyl-4-(pyrrolidin-1-ylmethyl) quinolin-2-ol

To a solution of 8-bromo-4-(pyrrolidin-1-ylmethyl) quinolin-2-ol (1.10 g, 3.58 mmol, 1.0 eq) in THF (10.0 mL) and NMP (2.00 mL) were added Fe (acac) 3 (632 mg, 1.79 mmol, 0.5 eq) and cyclobutylmagnesium bromide (0.5 M in THF) (35.8 mL, 17.9 mmol, 5.0 eq) at 0° C. The mixture was stirred under N₂ at 25° C. for 8 h. The reaction mixture was quenched with saturated aqueous NH₄Cl solution (50 mL) and extracted with EA (20 mL×3). The organic layer was washed with brine (50 mL), dried over MgSO₄, filtered and concentrated under reduced pressure. The crude product was purified by flash column chromatography on silica gel (MeOH in DCM, 4% v/v) to obtain 8-cyclobutyl-4-(pyrrolidin-1-ylmethyl) quinolin-2-ol (690 mg, yield 68%) as a yellow solid. LC-MS (ESI): mass calced for: C₁₈H₂₂N₂O, 282.39; m/z found, 283.2 [M+H]⁺. ¹H NMR (400 MHZ, DMSO-d₆) δ 10.35 (s, 1H), 7.76 (d, J=8.0 Hz, 1H), 7.43 (d, J=7.4 Hz, 1H), 7.18 (t, J=7.8 Hz, 1H), 6.51 (s, 1H), 4.09-4.01 (m, 1H), 3.77 (s, 2H), 2.52 (s, 4H), 2.41 (t, J=7.2 Hz, 2H), 2.07-2.00 (m, 4H), 1.71 (s, 4H).

### Step F: 8-cyclobutyl-4-(pyrrolidin-1-ylmethyl) quinolin-2-yl trifluoromethanesulfonate

To a solution of 8-cyclobutyl-4-(pyrrolidin-1-ylmethyl) quinolin-2-ol (100 mg, 354 μmol, 1.0 eq) in DCM (5.00 mL) were added pyridine (140 mg, 1.77 mmol, 5.0 eq) and Tf₂O (200 mg, 708 μmol, 2.0 eq) at 0° C. The mixture was stirred at 30° C. for 3 h. The reaction mixture was quenched with water (10 mL) and extracted with DCM (10 mL×3). The organic layer was washed with brine (20 mL), dried over MgSO₄, filtered and concentrated under reduced pressure. The crude product was purified by Prep-TLC(DCM/MeOH=20/1 v/v) to obtain 8-cyclobutyl-4-(pyrrolidin-1-ylmethyl) quinolin-2-yl trifluoromethanesulfonate (140 mg, yield 95%) as a yellow oil. LC-MS (ESI): mass calced for: C₁₉H₂₁F₃N₂O₃S, 414.44; m/z found, 415.2 [M+H]⁺. ¹H NMR (400 MHZ, DMSO-d₆) δ 8.17 (d, J=8.0 Hz, 1H), 7.80 (d, J=7.2 Hz, 1H), 7.75-7.68 (m, 1H), 7.58 (s, 1H), 4.34-4.22 (m, 1H), 4.14 (s, 2H), 2.57 (s, 4H), 2.47-2.39 (m, 2H), 2.25-2.14 (m, 2H), 2.10-2.04 (m, 1H), 1.89-1.82 (m, 1H), 1.74 (s, 4H).

### Intermediate 90:4-(bromomethyl)-2-chloro-8-cyclopropoxy-1,5-naphthyridine

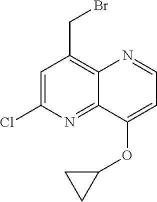 [SMILES: Clc1cc(CBr)c2nccc(OC3CC3)c2n1]

### Step A: 2-chloro-8-cyclopropoxy-4-methyl-1,5-naphthyridine

To a solution of 6-chloro-8-methyl-1,5-naphthyridin-4-ol (300 mg, 1.54 mmol, 1.0 eq) in DMF (6.00 mL) were added Cs₂CO₃ (1.51 g, 4.62 mmol, 3.0 eq) and bromocyclopropane (1.68 g, 13.9 mmol, 9.0 eq). The mixture was stirred at 140° C. for 16 h. After cooled to room temperature, the reaction mixture was quenched with saturated aqueous NH₄Cl solution (15 mL) and extracted with EA (10 mL×3). The organic layer was washed with brine (20 mL×4), dried over MgSO₄, filtered and concentrated under reduced pressure. The crude product was purified by flash column chromatography on silica gel (ethyl acetate in petroleum ether, 30% v/v) to obtain 2-chloro-8-cyclopropoxy-4-methyl-1,5-naphthyridine (60.0 mg, yield 16%) as a yellow oil. LC-MS (ESI): mass calced for: C₁₂H₁₁ClN₂O, 234.68; m/z found, 235.1 [M+H]⁺.

### Step B: 4-(bromomethyl)-2-chloro-8-cyclopropoxy-1,5-naphthyridine

To a solution of 2-chloro-8-cyclopropoxy-4-methyl-1,5-naphthyridine (60.0 mg, 256 μmol, 1.0 eq) in CCl₄ (7.00 mL) were added NBS (50.1 mg, 281 μmol, 1.1 eq) and Benzoyl peroxide (6.19 mg, 25.6 μmol, 0.1 eq). The mixture was stirred at 90° C. for 8 h. After cooled to room temperature, the reaction mixture was quenched with water (10 mL) and extracted with DCM (10 mL×3). The organic layer was dried over MgSO₄, filtered and concentrated under reduced pressure. The crude product was purified by Prep-TLC(PE/EA=2/1 v/v) to obtain 4-(bromomethyl)-2-chloro-8-cyclopropoxy-1,5-naphthyridine (20.0 mg, yield 24%) as a white solid. LC-MS (ESI): mass calced for: C₁₂H₁₀BrClN₂O, 313.58; m/z found, 314.2 [M+H]⁺. ¹H NMR (400 MHZ, DMSO-d₆) δ 9.00 (d, J=5.2 Hz, 1H), 8.08 (s, 1H), 7.69 (d, J=5.2 Hz, 1H), 5.22 (s, 2H), 4.27-4.22 (m, 1H), 1.04-0.99 (m, 2H), 0.96-0.91 (m, 2H).

### Intermediate 91:6-chloro-8-(pyrrolidin-1-ylmethyl)-1,5-naphthyridin-4-yl trifluoromethane sulfonate

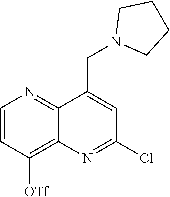 [SMILES: O=S(=O)(Oc1ccnc2c(CN3CCCC3)cc(Cl)nc12)C(F)(F)F]

### Step A: 5-(((6-chloro-4-methylpyridin-3-yl)amino)methylene)-2,2-dimethyl-1,3-dioxane-4,6-dione

A mixture of 6-chloro-4-methylpyridin-3-amine (12.5 g, 87.7 mmol, 1.0 eq) and 2,2-dimethyl-1,3-dioxane-4,6-dione (12.6 g, 87.7 mmol, 1.0 eq) was heated to 100° C. and the mixture was stirred for 10 min until the materials melted. Then CH (OMe) 3 (125 mL) was added to above mixture and the resulting reaction mixture was stirred at 100° C. for 30 min. The solution was cooled to room temperature and solid precipitated. After filtration, the cake was washd with MTBE (100 mL) and dried to get 5-(((6-chloro-4-methylpyridin-3-yl)amino)methylene)-2,2-dimethyl-1,3-dioxane-4,6-dione (20.0 g, yield 77%) as a white solid. LC-MS (ESI): mass calced for: C₁₃H₁₃ClN₂O₄, 296.06; m/z found, 297.1 [M+H]⁺. ¹H NMR (400 MHZ, DMSO-d₆) δ 11.15 (s, 1H), 8.62 (s, 1H), 8.57 (s, 1H), 7.54 (s, 1H), 2.35 (s, 3H), 1.69 (s, 6H).

### Step B: 6-chloro-8-methyl-1,5-naphthyridin-4-ol

The solvent diphenylether (230 mL) was heated to 220° C. and 5-(((6-chloro-4-methylpyridin-3-yl)amino)methylene)-2,2-dimethyl-1,3-dioxane-4,6-dione (5.00 g, 16.9 mmol, 1.0 eq) was added to diphenylether. The mixture was stirred at 220° C. for 30 min. The reaction mixture was cooled to room temperature and solid precipitated. After filtration, the filter cake was washed with MTBE (100 mL) and dried to obtain product 6-chloro-8-methyl-1,5-naphthyridin-4-ol (1.50 g, yield 46%) as a yellow solid. LC-MS (ESI): mass calced for: C₉H₇ClN₂O, 194.02; m/z found, 195.1 [M+H]⁺.

¹H NMR (400 MHZ, DMSO-d₆) δ 11.46 (s, 1H), 7.96 (s, 1H), 7.66 (s, 1H), 6.36 (s, 1H), 2.55 (s, 3H).

### Step C: 6-chloro-8-methyl-1,5-naphthyridin-4-yl trifluoromethanesulfonate

To a solution of 6-chloro-8-methyl-1,5-naphthyridin-4-ol (1.00 g, 5.14 mmol, 1.0 eq) and TEA (1.04 g, 1.43 mL, 10.3 mmol, 2.0 eq) in DCM (10.0 mL) was added Tf20 (2.17 g, 1.29 mL, 7.71 mmol, 1.5 eq) at 0° C. . . . The reaction mixture was stirred at 25° C. for 2 h. The mixture was diluted with DCM (50 mL), washed with brine (20 mL×2), dried over anhydrous sodium sulfate, filtered and concentrated under reduced pressure. The residue was purified by flash column chromatography on silica gel (PE/EA=5/1 v/v) to give 6-chloro-8-methyl-1,5-naphthyridin-4-yl trifluoromethanesulfonate (1.30 g, yield 78%) as a yellow oil. LC-MS (ESI): mass calced for: C₁₀H₆ClF₃N₂O₃S, 325.97; m/z found, 327.2 [M+H]⁺. ¹H NMR (400 MHZ, DMSO-d₆) δ 9.19 (d, J=5.0 Hz, 1H), 8.10 (d, J=5.0 Hz, 1H), 7.98 (s, 1H), 2.80 (s, 3H).

### Step D: (bromomethyl)-6-chloro-1,5-naphthyridin-4-yl trifluoromethanesulfonate

To a solution of 6-chloro-8-methyl-1,5-naphthyridin-4-yl trifluoromethanesulfonate (870 mg, 2.66 mmol, 1.0 eq) and NBS (521 mg, 2.93 mmol, 1.1 eq) in CCl₄ (20.0 mL) was added Benzoyl peroxide (96.8 mg, 399 μmol, 0.15 eq). The reaction mixture was stirred under N₂ atmosphere at 90° C. for 2 h. After evaporation, the residue was diluted with EA (50 mL), washed with saturated aqueous Na₂SO₃ solution (50 mL×2) and brine (50 mL), dried over anhydrous sodium sulfate, filtered and concentrated under reduced pressure. The residue was purified by flash column chromatography on silica gel (PE/EA=2/1 v/v) to obtain 8-(bromomethyl)-6-chloro-1,5-naphthyridin-4-yl trifluoromethanesulfonate (400 mg, yield 37%) as a white solid. LC-MS (ESI): mass calced for: C₁₀HsBrClF3N₂O₃S, 403.88; m/z found, 405.2 [M+H]⁺. ¹H NMR (400 MHZ, DMSO-d₆) δ 9.27 (d, J=5.0 Hz, 1H), 8.26 (s, 1H), 8.17 (d, J=5.0 Hz, 1H), 5.20 (s, 2H).

### Step E: 6-chloro-8-(pyrrolidin-1-ylmethyl)-1,5-naphthyridin-4-yl trifluoromethanesulfonate

To a solution of 8-(bromomethyl)-6-chloro-1,5-naphthyridin-4-yl trifluoromethanesulfonate (1.10 g, 2.71 mmol, 1.0 eq) and DIPEA (1.05 g, 8.14 mmol, 3.0 eq) in DCM (10.0 mL) was added pyrrolidine (174 mg, 2.44 mmol, 0.9 eq). The reaction mixture was stirred at 25° C. for 1 h. After evaporation, the residue was purified by flash column chromatography on silica gel (DCM/MeOH=10/1 v/v) to obtain 6-chloro-8-(pyrrolidin-1-ylmethyl)-1,5-naphthyridin-4-yl trifluoromethanesulfonate (800 mg, yield 75%) as a yellow oil. LC-MS (ESI): mass calced for: C₁₄H₁₃ClF₃N₃O₃S, 395.03; m/z found, 396.1 [M+H]⁺. ¹H NMR (400 MHZ, DMSO-d₆) δ 9.20 (d, J=4.8 Hz, 1H), 8.13 (d, J=4.8 Hz, 1H), 7.96 (s, 1H), 4.37 (s, 2H), 2.67 (s, 4H), 1.80 (s, 4H).

### Intermediate 92:6-chloro-1-(oxetan-3-yl)-3-(trifluoromethyl)-1H-pyrazolo[3,4-b]pyridine-4-carbaldehyde

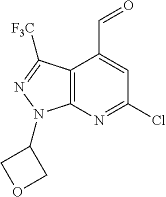 [SMILES: O=Cc1cc(Cl)nc2c1c(C(F)(F)F)nn2C1COC1]

### Step A: ethyl 6-chloro-3-iodo-1H-pyrazolo[3,4-b]pyridine-4-carboxylate

To a solution of ethyl 6-chloro-1H-pyrazolo[3,4-b]pyridine-4-carboxylate (5.00 g, 22.6 mmol, 1.0 eq) in DMF (80.0 mL) was added K₂CO₃ (3.12 g, 22.6 mmol, 1.0 eq) and the mixture was stirring at 25° C. for 10 min. Then diiodine (14.3 g, 56.5 mmol, 2.5 eq) was added to above mixture and the resulting mixture was stirred at 110° C. for 16 h. After cooled to room temperature, the mixture was quenched with saturated aqueous NaHCO₃ solution (60 mL) and extracted with EtOAc (150 mL×3). The organic layer was washed with saturated aqueous Na₂S₂O₃ solution (100 mL×3) and brine (100 mL), dried over anhydrous Na₂SO₄, filtered and concentrated under reduced pressure. The residue was purified by flash column chromatography on silica gel (PE/EA=1/1 v/v) to obtain ethyl 6-chloro-3-iodo-1H-pyrazolo[3,4-b]pyridine-4-carboxylate (4.40 g, yield 55%) as a white solid. LC-MS (ESI): mass calced for: C₉H₇ClIN₃O₂, 350.93; m/z found, 352.1 [M+H]⁺. ¹H NMR (400 MHZ, DMSO-d₆) δ 14.60 (s, 1H), 7.55 (s, 1H), 4.48 (q, J=7.2 Hz, 2H), 1.42 (t, J=7.2 Hz, 3H).

### Step B: ethyl 6-chloro-3-iodo-1-(oxetan-3-yl)-1H-pyrazolo[3,4-b]pyridine-4-carboxylate

To a solution of ethyl 6-chloro-3-iodo-1H-pyrazolo[3,4-b]pyridine-4-carboxylate (1.00 g, 2.84 mmol, 1.0 eq) in DMA (15.0 mL) were added K₂CO₃ (1.18 g, 8.53 mmol, 3.0 eq) and 3-iodooxetane (785 mg, 4.27 mmol, 1.5 eq) at 25° C. and the mixture was stirred at 80° C. for 16 h. After cooled to room temperature, the reaction mixture was quenched with water (20 mL) and extracted with EtOAc (30 mL×3). The organic layer was washed with brine (10 mL×3), dried over anhydrous Na₂SO₄, filtered and concentrated under reduced pressure. The residue was purified with flash column chromatography on silica gel (EA in PE, from 0% to 20% v/v) to afford ethyl 6-chloro-3-iodo-1-(oxetan-3-yl)-1H-pyrazolo[3,4-b]pyridine-4-carboxylate (540 mg, yield 47%) as a yellow solid. LC-MS (ESI): mass calced for: C₁₂HuClIN303, 406.95; m/z found, 408.1 [M+H]⁺. ¹H NMR (400 MHZ, DMSO-d₆) δ 7.61 (s, 1H), 6.12-6.05 (m, 1H), 5.03-4.99 (m, 4H), 4.48 (q, J=7.2 Hz, 2H), 1.42 (t, J=7.2 Hz, 3H).

### Step C: ethyl 6-chloro-1-(oxetan-3-yl)-3-(trifluoromethyl)-1H-pyrazolo[3,4-b]pyridine-4-carboxylate

To a solution of ethyl 6-chloro-3-iodo-1-(oxetan-3-yl)-1H-pyrazolo[3,4-b]pyridine-4-carboxylate (1.25 g, 3.07 mmol, 1.0 eq) in DMF (30.0 mL) were added ethyl methyl difluoro (fluoro-sulfonyl) acetate (1.77 g, 9.20 mmol, 3.0 eq) and CuI (1.75 g, 9.20 mmol, 3.0 eq) at 25° C. The mixture was stirred under N₂ at 80° C. for 16 h. After cooled to room temperature, the reaction mixture was filtered and the filtrate was diluted with EA (100 mL). The organic layer was washed with brine (50 mL×4), dried over anhydrous Na₂SO₄, filtered and concentrated under reduced pressure. The residue was purified with flash column chromatography on silica gel (EA in PE, from 0% to 20% v/v) to afford ethyl 6-chloro-1-(oxetan-3-yl)-3-(trifluoromethyl)-1H-pyrazolo[3,4-b]pyridine-4-carboxylate (900 mg, yield 84%) as a yellow solid. LC-MS (ESI): mass calced for: C₁₃H₁₁ClF₃N₃O₃, 349.04; m/z found, 350.1 [M+H]⁺. ¹H NMR (400 MHZ, DMSO-d₆) δ 7.86 (s, 1H), 6.25-6.17 (m, 1H), 5.06-5.02 (m, 4H), 4.43 (q, J=7.2 Hz, 2H), 1.35 (t, J=7.2 Hz, 3H).

### Step D: (6-chloro-1-(oxetan-3-yl)-3-(trifluoromethyl)-1H-pyrazolo[3,4-b]pyridin-4-yl) methanol

To a solution of ethyl 6-chloro-1-(oxetan-3-yl)-3-(trifluoromethyl)-1H-pyrazolo[3,4-b]pyridine-4-carboxylate (400 mg, 1.14 mmol, 1.0 eq) in THF (10.0 mL) were added LiAlH₄ (52.1 mg, 1.37 mmol, 1.2 eq) under N₂ at 0° C. for 10 min. The mixture was quenched with saturated aqueous NH₄Cl solution (5 mL), diluted with water (5 mL), and extracted with EtOAc (10 mL×3). The organic layer was dried over MgSO₄, filtered and concentrated under reduced pressure to obtain crude product (6-chloro-1-(oxetan-3-yl)-3-(trifluoromethyl)-1H-pyrazolo[3,4-b]pyridin-4-yl) methanol (200 mg, yield 57%) as a brown solid. LC-MS (ESI): mass calced for: C₁₁H₉ClF₃N₃O₂, 307.1; m/z found, 308.1 [M+H]⁺.

### Step E: 6-chloro-1-(oxetan-3-yl)-3-(trifluoromethyl)-1H-pyrazolo[3,4-b]pyridine-4-carbaldehyde

To a solution of (6-chloro-1-(oxetan-3-yl)-3-(trifluoromethyl)-1H-pyrazolo[3,4-b]pyridin-4-yl) methanol (300 mg, 975 μmol, 1.0 eq) in DMSO (10.0 mL) were added IBX (546 mg, 1.95 mmol, 2.0 eq) at 25° C. and the mixture was stirred at room temperature for 2 h. The mixture was quenched with saturated aqueous Na₂S₂O₃ solution (5 mL), diluted with water (5 mL), and extracted with EtOAc (10 mL×3). The organic layer was dried over MgSO₄, filtered and concentrated under reduced pressure. The residue was purified with flash column chromatography on silica gel (ethyl acetate in petroleum ether, from 0% to 100% v/v) to afford 6-chloro-1-(oxetan-3-yl)-3-(trifluoromethyl)-1H-pyrazolo[3,4-b]pyridine-4-carbaldehyde (40.0 mg, yield 12%) as a yellow solid. LC-MS (ESI): mass calced for: C₁₁H₇ClF₃N₃O₂, 305.1; m/z found, 306.1 [M+H]⁺.

### Intermediate 93:6-chloro-3-methyl-1-(oxetan-3-yl)-1H-pyrazolo[3,4-b]pyridine-4-carbaldehyde

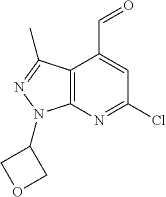 [SMILES: Cc1nn(C2COC2)c2nc(Cl)cc(C=O)c12]

### Step A: ethyl 6-hydroxy-3-methyl-1H-pyrazolo[3,4-b]pyridine-4-carboxylate

To a solution of 5-methyl-1H-pyrazol-3-amine (4.0 g, 41.2 mmol, 1.0 eq) in H₂O (6.0 mL) were added sodium (Z)-1,4-diethoxy-1,4-dioxobut-2-en-2-olate (8.66 g, 41.2 mmol, 1.0 eq) and AcOH (2.0 mL). The mixture was stirring at 85° C. for 16 h. After cooled to room temperature, the reaction mixture was filtered and the filter cake was dried to give ethyl 6-hydroxy-3-methyl-1H-pyrazolo[3,4-b]pyridine-4-carboxylate (2.30 g, yield 25%) as a brown solid. LC-MS (ESI): mass calced for: C₁₀H₁₁N₃O₃, 221.08; m/z found, 222.1 [M+H]⁺. ¹H NMR (400 MHZ, DMSO-d₆) δ 13.06 (s, 1H), 11.95 (s, 1H), 6.46 (s, 1H), 4.35 (q, J=7.2 Hz, 2H), 2.46 (s, 3H), 1.33 (t, J=7.2 Hz, 3H).

### Step B: ethyl 6-chloro-3-methyl-1H-pyrazolo[3,4-b]pyridine-4-carboxylate

To a solution of ethyl 6-hydroxy-3-methyl-1H-pyrazolo[3,4-b]pyridine-4-carboxylate (11.5 g, 52.0 mmol, 1.0 eq) in Toluene (120 mL) was added DBU (15.8 g, 104 mmol, 2.0 eq) and the mixture was stirring at 25° C. for 10 min. Then phosphoryl trichloride (12.0 g, 78.0 mmol, 1.5 eq) was added to above mixture and the resulting mixture was stirred at 100° C. for 2 h. After cooled to room temperature, the mixture was quenched with saturated aqueous NaHCO₃ solution (150 mL) and extracted with EtOAc (300 mL×3). The organic layer was washed with brine (300 mL), dried over Na₂SO₄, filtered and concentrated in vacuum. The residue was purified by flash column chromatography on silica gel (PE: ethyl acetate=1:1) to obtain ethyl 6-chloro-3-methyl-1H-pyrazolo[3,4-b]pyridine-4-carboxylate (3.0 g, yield 24%) as a white solid. LC-MS (ESI): mass calced for: C₁₀H₁₀ClN₃O₂, 239.05; m/z found, 240.0 [M+H]⁺. ¹H NMR (400 MHZ, DMSO-d₆) δ 6.46 (s, 1H), 4.35 (q, J=7.2 Hz, 2H), 2.45 (s, 3H), 1.33 (t, J=7.2 Hz, 3H).

### Step C: methyl 6-chloro-3-methyl-1-(oxetan-3-yl)-1H-pyrazolo[3,4-b]pyridine-4-carboxylate

To a solution of ethyl 6-chloro-3-methyl-1H-pyrazolo[3,4-b]pyridine-4-carboxylate (500 mg, 2.09 mmol, 1.0 eq) in DMF (15.0 mL) was added Cs₂CO₃ (1.36 g, 4.18 mmol, 2.0 eq) and the mixture was stirring at 25° C. for 10 min. Then 3-iodooxetane (576 mg, 3.13 mmol, 1.5 eq) was added to above mixture and the mixture was stirred at 100° C. for 10 h. After cooled to room temperature, MeI (318 mg, 140 μL, 2.24 mmol, 2.0 eq) was added to above mixture and the mixture was stirred at 25° C. for 1 h. The mixture was poured into water (30 mL) and extracted with EA (30 mL×3). The organic layer was washed with brine (30 mL×4), dried over anhydrous Na₂SO₄, filtered and concentrated under reduced pressure. The crude product was purified by Prep-TLC(PE/EA=5/1 v/v) to give 6-chloro-3-methyl-1-(oxetan-3-yl)-1H-pyrazolo[3,4-b]pyridine-4-carboxylic acid (200.0 mg, yield 40%) as a white solid. LC-MS (ESI): mass calced for: C₁₂H₁₂ClN₃O₃, 281.06; m/z found, 282.06 [M+H]⁺. ¹H NMR (400 MHZ, DMSO-d₆) δ 7.61 (s, 1H), 6.06 (p, J=7.0 Hz, 1H), 5.08-4.92 (m, 4H), 3.98 (s, 3H), 2.65 (s, 3H).

### Step D: (6-chloro-3-methyl-1-(oxetan-3-yl)-1H-pyrazolo[3,4-b]pyridin-4-yl) methanol

To a solution of methyl 6-chloro-3-methyl-1-(oxetan-3-yl)-1H-pyrazolo[3,4-b]pyridine-4-carboxylate (580 mg, 2.06 mmol, 1.0 eq) in THF (30.0 mL) was added LiAlH₄ (156 mg, 4.12 mmol, 2.0 eq) in portions at 0° C. The mixture was stirred at 25° C. for 2 h. The residue was quenched with Na₂SO₄·10H₂O and filtrated. The filtrate was concentrated under reduced pressure and purified by Prep-TLC(DCM/MeOH=10/1 v/v) to give (6-chloro-3-methyl-1-(oxetan-3-yl)-1H-pyrazolo[3,4-b]pyridin-4-yl) methanol (520.2 mg, yield 100%) as a yellow solid. LC-MS (ESI): mass calced for: C₁₁H₁₂ClN₃O₂, 253.06; m/z found, 254.06 [M+H]⁺. ¹H NMR (400 MHZ, DMSO-d₆) δ 7.22 (s, 1H), 5.98 (p, J=7.0 Hz, 1H), 5.71 (t, J=5.6 Hz, 1H), 5.06-4.93 (m, 6H), 2.63 (s, 3H).

### Step E: 6-chloro-3-methyl-1-(oxetan-3-yl)-1H-pyrazolo[3,4-b]pyridine-4-carbaldehyde

To a stirred mixture of (6-chloro-3-methyl-1-(oxetan-3-yl)-1H-pyrazolo[3,4-b]pyridin-4-yl) methanol (150 mg, 591 μmol, 1.0 eq) in DMSO (10.0 mL) was added IBX (248 mg, 887 μmol, 1.5 eq). The resulting mixture was stirred at 25° C. for 1 h. The reaction mixture was quenched with saturated aqueous Na₂SO₃ solution (30 mL) and extracted with EtOAc (20 mL×3). The combined organic layers were washed with brine (50 mL×3), dried over anhydrous Na₂SO₄ and concentrated under reduced pressure. The residue was purified by flash column chromatography on silica gel (PE/EtOAc=5/1 v/v) to give 6-chloro-3-methyl-1-(oxetan-3-yl)-1H-pyrazolo[3,4-b]pyridine-4-carbaldehyde (110 mg, yield 74%) as a yellow solid. LC-MS (ESI): mass calced for: C₁₁H₁₀ClN₃O₂, 251.05; m/z found, 252.0 [M+H]⁺.

### Intermediate 94:6-chloro-1-(1-methyl-1H-pyrazol-4-yl)-1H-pyrrolo[2,3-b]pyridine-4-carbaldehyde

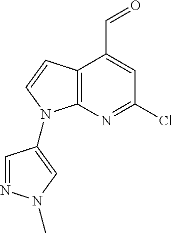 [SMILES: Cn1cc(-n2ccc3c(C=O)cc(Cl)nc32)cn1]

### Step A: methyl 6-chloro-1-(1-methyl-1H-pyrazol-4-yl)-1H-pyrrolo[2,3-b]pyridine-4-carboxylate

To a suspension of methyl 6-chloro-1H-pyrrolo[2,3-b]pyridine-4-carboxylate (500 mg, 2.37 mmol, 1.0 eq), (1-methyl-1H-pyrazol-4-yl) boronic acid (897 mg, 7.12 mmol, 3.0 eq), Sodium carbonate (1.26 g, 11.9 mmol, 5.0 eq) in 1,2-dichloroethane (50.0 mL) were added Cu (OAc)₂ (862 mg, 4.75 mmol, 2.0 eq) and 2,2′-Bipyridine (742 mg, 4.75 mmol, 2.0 eq) and the mixture was stirred under 02 (1 atm) at 90° C. for 24 h. After cooled to room temperature, the mixture was filtered and the filtrate was diluted with ethyl acetate (100 mL). The organic layer was washed with saturated aqueous ammonium chloride solution (50 mL), water (50 mL) and saturated brine (50 mL), and dried over magnesium sulfate. The filtrate was concentrated under reduced pressure. The residue was purified by flash column chromatography on silica gel (PE/EA=3/1 v/v) to provide methyl 6-chloro-1-(1-methyl-1H-pyrazol-4-yl)-1H-pyrrolo[2,3-b]pyridine-4-carboxylate (40.0 mg, yield 6%) as a yellow solid. LC-MS (ESI): mass calced for: C₁₃H₁₁ClN₄O₂, 290.06; m/z found, 291.1 [M+H]⁺.

### Step B: (6-chloro-1-(1-methyl-1H-pyrazol-4-yl)-1H-pyrrolo[2,3-b]pyridin-4-yl) methanol

To a solution of methyl 6-chloro-1-(1-methyl-1H-pyrazol-4-yl)-1H-pyrrolo[2,3-b]pyridine-4-carboxylate (124 mg, 427 μmol, 1.0 eq) in THF (10.0 mL) was added Aluminum lithium hydride (32.4 mg, 853 μmol, 2.0 eq) at 0° C. The reaction mixture was stirred at 0° C. for 20 min. The reaction mixture was quenched with Na₂SO₄·10H₂O and stirred for 30 min. The mixture was filtered and the filtrate was diluted with water (10 mL), and extracted with ethyl acetate (10 mL×3). The combined organic extracts were washed with brine (10 mL), dried over anhydrous sodium sulfate, filtered, and concentrated under reduced pressure to give (6-chloro-1-(1-methyl-1H-pyrazol-4-yl)-1H-pyrrolo[2,3-b]pyridin-4-yl) methanol (85.0 mg, yield 76%) as a yellow oil. The crude product was directly used in next step without further purification. LC-MS (ESI): mass calced for: C₁₂H₁₁ClN₄O, 262.06; m/z found, 263.2 [M+H]⁺.

### Step C: 6-chloro-1-(1-methyl-1H-pyrazol-4-yl)-1H-pyrrolo[2,3-b]pyridine-4-carbaldehyde

To a solution of (6-chloro-1-(1-methyl-1H-pyrazol-4-yl)-1H-pyrrolo[2,3-b]pyridin-4-yl) methanol (85.0 mg, 324 μmol, 1.0 eq) in DMSO (8.00 mL) was added IBX (Purity 45% W.t) (227 mg, 809 μmol, 2.5 eq) at room temperature. The reaction mixture was stirred at 30° C. for 30 min. The reaction mixture was diluted with water (20 mL) and extracted with ethyl acetate (20 mL×3). The combined organic extracts were washed with saturated aqueous Na₂S₂O₃ solution (30 mL×2), saturated aqueous NaHCO₃ solution (30 mL×2) and brine (30 mL), dried over anhydrous sodium sulfate, filtered and concentrated in vacuum. The crude product was purified by Prep-TLC(PE/EA=1/1 v/v) to provide 6-chloro-1-(1-methyl-1H-pyrazol-4-yl)-1H-pyrrolo[2,3-b]pyridine-4-carbaldehyde (58.0 mg, yield 69%) as a yellow solid. LC-MS (ESI): mass calced for: C₁₂H₉ClN₄O, 260.05; m/z found, 261.1 [M+H]⁺.

### Intermediate 95:4-(bromomethyl)-2-chloro-8-(difluoromethoxy)-1,5-naphthyridine

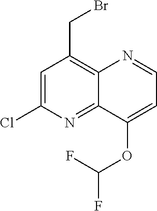 [SMILES: FC(F)Oc1ccnc2c(CBr)cc(Cl)nc12]

### Step A: 2-chloro-8-(difluoromethoxy)-4-methyl-1,5-naphthyridine

To a solution of 6-chloro-8-methyl-1,5-naphthyridin-4-ol (100 mg, 514 μmol, 1.0 eq) in DMF (3.00 mL) were added K₂CO₃ (178 mg, 1.28 mmol, 2.5 eq) and sodium 2-chloro-2,2-difluoroacetate (118 mg, 771 μmol, 1.5 eq). The mixture was stirred at 80° C. for 3 h. After cooled to room temperature, the reaction mixture was quenched with saturated aqueous NH₄Cl solution (8 mL) and extracted with EA (5 mL×3). The organic layer was washed with brine (5 mL×4), dried over MgSO₄, filtered and concentrated under reduced pressure. The crude product was purified by Prep-TLC(PE/EA=1/1 v/v) to obtain 2-chloro-8-(difluoromethoxy)-4-methyl-1,5-naphthyridine (84.0 mg, yield 66%) as a white solid. LC-MS (ESI): mass calced for: C₁₀H₇CIF2N₂O, 244.63; m/z found, 245.3 [M+H]⁺. ¹H NMR (400 MHZ, DMSO-d₆) δ 8.97 (d, J=5.2 Hz, 1H), 7.83 (d, J=0.8 Hz, 1H), 7.88-7.50 (dd, J=72.6, 1H), 7.59 (d, J=5.2 Hz, 1H), 2.75 (d, J=0.8 Hz, 3H).

### Step B: 4-(bromomethyl)-2-chloro-8-(difluoromethoxy)-1,5-naphthyridine

To a solution of 2-chloro-8-(difluoromethoxy)-4-methyl-1,5-naphthyridine (68.0 mg, 278 μmol, 1.0 eq) in CCl₄ (7.00 mL) were added NBS (54.4 mg, 306 μmol, 1.1 eq) and Benzoyl peroxide (6.73 mg, 27.8 μmol, 0.1 eq). The mixture was stirred at 90° C. for 8 h. After cooled to room temperature, the reaction mixture was quenched with water (10 mL) and extracted with DCM (10 mL×3). The organic layer was dried over MgSO₄, filtered and concentrated under reduced pressure. The crude product was purified by Prep-TLC(PE/EA=4/1 v/v) to obtain 4-(bromomethyl)-2-chloro-8-(difluoromethoxy)-1,5-naphthyridine (66.0 mg, yield 73%) as a yellow solid. LC-MS (ESI): mass calced for: C₁₀H₆BrCIF2N₂O, 323.52; m/z found, 324.1 [M+H]⁺.

### Intermediate 96:4-(bromomethyl)-2-chloro-8-(oxetan-3-yloxy)-1,5-naphthyridine

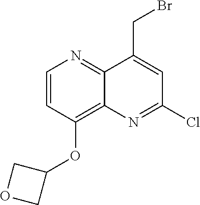 [SMILES: Clc1cc(CBr)c2nccc(OC3COC3)c2n1]

### Step A: 2-chloro-4-methyl-8-(oxetan-3-yloxy)-1,5-naphthyridine

To a solution of 6-chloro-8-methyl-1,5-naphthyridin-4-ol (500 mg, 2.57 mmol, 1.0 eq) in DMF (10.0 mL) were added 3-iodooxetane (1.42 g, 7.71 mmol, 3.0 eq) and Cs₂CO₃ (2.51 g, 7.71 mmol, 3.0 eq). The reaction mixture was stirred at 80° C. for 16 h. After cooled to room temperature, the reaction mixture was diluted with EA (100 mL), washed with water (30 mL×4) and brine (30 mL), dried over anhydrous sodium sulfate, filtered and concentrated under reduced pressure. The residue was purified by flash column chromatography on silica gel (PE/EA=2/1 v/v) to obtain 2-chloro-4-methyl-8-(oxetan-3-yloxy)-1,5-naphthyridine (330 mg, yield 51%) as a yellow oil. LC-MS (ESI): mass calced for: C₁₂H₁₁ClN₂O₂, 250.05; m/z found, 251.1 [M+H]⁺. ¹H NMR (400 MHz, DMSO-d₆) δ 8.79 (d, J=5.2 Hz, 1H), 7.77 (d, J=0.8 Hz, 1H), 6.98 (d, J=5.2 Hz, 1H), 5.65-5.47 (m, 1H), 5.05 (t, J=7.0 Hz, 2H), 4.72-4.69 (m, 2H), 2.72 (s, 3H).

### Step B: 4-(bromomethyl)-2-chloro-8-(oxetan-3-yloxy)-1,5-naphthyridine

To a solution of 2-chloro-4-methyl-8-(oxetan-3-yloxy)-1,5-naphthyridine (300 mg, 1.20 mmol, 1.0 eq) and NBS (234 mg, 1.32 mmol, 1.1 eq) in CCl₄ (20.00 mL) was added AIBN (19.7 mg, 120 μmol, 0.1 eq) under N₂ atmosphere. The reaction mixture was stirred under N₂ atmosphere at 90° C. for 3 h. After cooled to room temperature, the reaction mixture was concentrated under reduced pressure and the residue was purified by Prep-TLC(PE/EA=2/1 v/v) to obtain 4-(bromomethyl)-2-chloro-8-(oxetan-3-yloxy)-1,5-naphthyridine (40.0 mg, yield 10%) as a yellow oil. LC-MS (ESI): mass calced for: C₁₂H₁₀BrClN₂O₂, 327.96; m/z found, 329.0 [M+H]⁺.

### Intermediate 97:6-chloro-1-(3-methoxycyclobutyl)-4-(pyrrolidin-1-ylmethyl)-1H-pyrrolo[2,3-b]pyridine

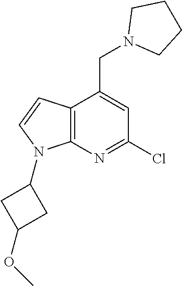 [SMILES: COC1CC(n2ccc3c(CN4CCCC4)cc(Cl)nc32)C1]

To a solution of 6-chloro-4-(pyrrolidin-1-ylmethyl)-1H-pyrrolo[2,3-b]pyridine (50.0 mg, 212 μmol, 1.0 eq) in DMF (3.00 mL) were added Cs₂CO₃ (207 mg, 636 μmol, 3.0 eq) and 3-methoxycyclobutyl methanesulfonate (115 mg, 636 μmol, 3.0 eq) at 25° C. The mixture was stirred at 80° C. for 16 h. After cooled to room temperature, the reaction mixture was quenched with water (10 mL) and extracted with EtOAc (20 mL×3). The organic layer was washed with brine (15 mL×4), dried over anhydrous Na₂SO₄, filtered and concentrated under reduced pressure. The residue was purified by Prep-TLC(DCM/MeOH=10/1 v/v) to afford 6-chloro-1-(3-methoxycyclobutyl)-4-(pyrrolidin-1-ylmethyl)-1H-pyrrolo[2,3-b]pyridine (30.0 mg, yield 44%) as a yellow solid. LC-MS (ESI): mass calced for: C₁₇H₂₂ClN₃O, 319.15; m/z found, 320.0 [M+H]⁺.

### Intermediate 98:1-(3-(6-chloro-4-(pyrrolidin-1-ylmethyl)-1H-pyrrolo[2,3-b]pyridin-1-yl) azetidin-1-yl) ethan-1-one

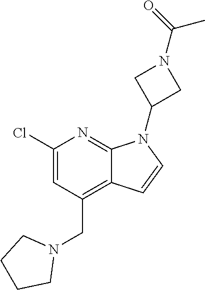 [SMILES: CC(=O)N1CC(n2ccc3c(CN4CCCC4)cc(Cl)nc32)C1]

### Step A: tert-butyl 3-(6-chloro-4-(pyrrolidin-1-ylmethyl)-1H-pyrrolo[2,3-b]pyridin-1-yl) azetidine-1-carboxylate

To a solution of 6-chloro-4-(pyrrolidin-1-ylmethyl)-1H-pyrrolo[2,3-b]pyridine (400 mg, 1.7 mmol, 1.0 eq) in DMF (10 mL) were added Cs₂CO₃ (1.7 g, 5.1 mmol, 3.0 eq) and tert-butyl 3-bromoazetidine-1-carboxylate (481 mg, 2.0 mmol, 1.2 eq) at 25° C. The reaction mixture was stirred at 80° C. for 16 h. After cooled to room temperature, the reaction mixture was quenched with H₂O (20 mL) and extracted with EtOAc (20 mL×3). The organic layer was washed with brine (10 mL), dried over anhydrous Na₂SO₄, filtered and concentrated under reduced pressure. The residue was purified by flash column chromatography on silica gel (DCM/MeOH=10/1 v/v) to give tert-butyl 3-(6-chloro-4-(pyrrolidin-1-ylmethyl)-1H-pyrrolo[2,3-b]pyridin-1-yl) azetidine-1-carboxylate (500 mg, yield 75%) as a white solid. LC-MS (ESI): mass calcd. for C₂₀H₂₇ClN₄O₂, 390.2; m/z found, 391.2 [M+H]⁺.

### Step B: 1-(azetidin-3-yl)-6-chloro-4-(pyrrolidin-1-ylmethyl)-1H-pyrrolo[2,3-b]pyridine

To a solution of tert-butyl 3-(6-chloro-4-(pyrrolidin-1-ylmethyl)-1H-pyrrolo[2,3-b]pyridin-1-yl) azetidine-1-carboxylate (200 mg, 512 μmol, 1.0 eq) in DCM (6.0 mL) was added TFA (2.0 mL) at 25° C. The reaction mixture was stirred at room temperature for 1 h. The reaction mixture was concentrated under reduced pressure to get 1-(azetidin-3-yl)-6-chloro-4-(pyrrolidin-1-ylmethyl)-1H-pyrrolo[2,3-b]pyridine (130 mg, yield 87%) as a yellow oil. The crude product was directly used in next step without further purification. LC-MS (ESI): mass calcd. for C₁₅H₁₉ClN₄, 290.1; m/z found, 291.1 [M+H]⁺.

### Step C: 1-(3-(6-chloro-4-(pyrrolidin-1-ylmethyl)-1H-pyrrolo[2,3-b]pyridin-1-yl) azetidin-1-yl) ethan-1-one

To a solution of 1-(azetidin-3-yl)-6-chloro-4-(pyrrolidin-1-ylmethyl)-1H-pyrrolo[2,3-b]pyridine (100 mg, 344 μmol, 1.0 eq) in DCM (5.0 mL) were added TEA (52 mg, 516 μmol, 1.5 eq) and AcOH (53 mg, 516 μmol, 1.0 eq) at 25° C. The reaction mixture was stirred at room temperature for 1 h. The reaction mixture was quenched with water (5 mL) and extracted with DCM (5 mL×3). The organic layer was washed with brine (5 mL), dried over anhydrous Na₂SO₄, filtered and concentrated under reduced pressure. The residue was purified by Prep-TLC(DCM/MeOH=10/1 v/v) to give 1-(3-(6-chloro-4-(pyrrolidin-1-ylmethyl)-1H-pyrrolo[2,3-b]pyridin-1-yl) azetidin-1-yl) ethan-1-one (20 mg, yield 18%) as a brown solid. LC-MS (ESI): mass calcd. for C₁₇H₂₁ClN₄O, 332.1; m/z found, 333.1 [M+H]⁺.

### Intermediate 99:6-chloro-4-((2-(fluoromethyl) pyrrolidin-1-yl)methyl)-1-(oxetan-3-yl)-1H-pyrrolo[2,3-b]pyridine

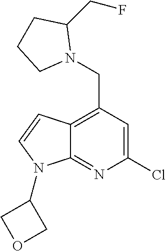 [SMILES: FCC1CCCN1Cc1cc(Cl)nc2c1ccn2C1COC1]

### Step A: (1-((6-chloro-1-(oxetan-3-yl)-1H-pyrrolo[2,3-b]pyridin-4-yl)methyl) pyrrolidin-2-yl) methanol

To a solution of 6-chloro-1-(oxetan-3-yl)-1H-pyrrolo[2,3-b]pyridine-4-carbaldehyde (200 mg, 845 μmol, 1.0 eq) and pyrrolidin-2-ylmethanol (103 mg, 1.01 mmol, 1.2 eq) in DCM (4.00 mL) was added AcOH (0.1 mL) and the mixture was stirred at room temperature for 1 h. Then Sodium triacetoxyborohydride (448 mg, 2.11 mmol, 2.5 eq) was added to above mixture and the mixture was stirred at room temperature overnight. The mixture was poured into water (6 mL) and extracted with DCM (10 mL×3). The organic layer was dried over MgSO₄, filtered and concentrated under reduced pressure. The residue was purified by Prep-TLC(DCM/MeOH=10/1 v/v) to give (1-((6-chloro-1-(oxetan-3-yl)-1H-pyrrolo[2,3-b]pyridin-4-yl)methyl) pyrrolidin-2-yl) methanol (130 mg, yield 43%) as a yellow oil. LC-MS (ESI): mass calced for: C₁₆H₂OClN₃O₂, 321.1; m/z found, 322.2 [M+H]⁺.

### Step B: 6-chloro-4-((2-(fluoromethyl) pyrrolidin-1-yl)methyl)-1-(oxetan-3-yl)-1H-pyrrolo[2,3-b]pyridine

To a solution of (1-((6-chloro-1-(oxetan-3-yl)-1H-pyrrolo[2,3-b]pyridin-4-yl)methyl) pyrrolidin-2-yl) methanol (50.0 mg, 155 μmol, 1.0 eq) in DCM (3.00 mL) was added DAST (50.1 mg, 41.1 μL, 311 μmol, 2.0 eq) and the mixture was stirring at room temperature for 16 h. The mixture was poured into water (5 mL) and extracted with DCM (15 mL×3). The organic layer was dried over MgSO₄, filtered and concentrated under reduced pressure. The residue was purified by Prep-TLC(EA/PE=2/1 v/v) to obtain 6-chloro-4-((2-(fluoromethyl) pyrrolidin-1-yl)methyl)-1-(oxetan-3-yl)-1H-pyrrolo[2,3-b]pyridine (10.0 mg, yield 18%) as a yellow oil. LC-MS (ESI): mass calced for: C₁₆H₁₉CIFN30, 323.1; m/z found, 324.1 [M+H]⁺.

### Intermediate 100:7-(bromomethyl)-5-chloro-2-methyl-3-(oxetan-3-yl)-3H-imidazo[4,5-b]pyridine (100a) and 2-(bromomethyl)-5-chloro-7-methyl-3-(oxetan-3-yl)-3H-imidazo[4,5-b]pyridine (100b)

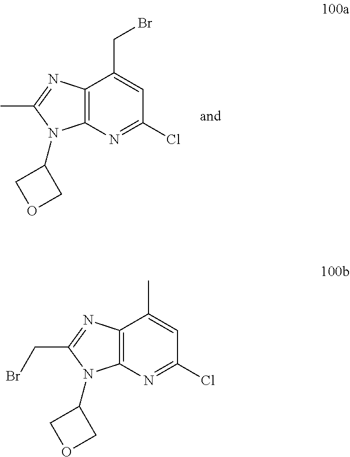 [SMILES: Cc1nc2c(CBr)cc(Cl)nc2n1C1COC1 ; Cc1cc(Cl)nc2c1nc(CBr)n2C1COC1]

### Step A: 6-chloro-4-methyl-3-nitro-N-(oxetan-3-yl) pyridin-2-amine

To a solution of 2,6-dichloro-4-methyl-3-nitropyridine (500 mg, 2.42 mmol, 1.0 eq) in THF (8 mL) was added Diisopropylethylamine (1.56 g, 2.09 mL, 12.1 mmol, 5.0 eq) at room temperature. Then a solution of oxetan-3-amine (177 mg, 2.42 mmol, 1.0 eq) in THF (2 mL) was dropwise added to above mixture at −40° C. and the resulting reaction mixture was stirring for 16 h at room temperature. The mixture was quenched with water (20 mL) and extracted with EtOAc (40 mL×3). The organic layers were washed with brine (40 mL), dried over anhydrous Na₂SO₄, filtered and concentrated under reduced pressure. The crude product was purified by flash column chromatography on silica gel (PE/EA=10/1 v/v) to provide 6-chloro-4-methyl-3-nitro-N-(oxetan-3-yl) pyridin-2-amine (180 mg, yield 31%) as a yellow solid. LC-MS (ESI): mass calced for: C₉H₁₀ClN₃O₃, 243.04; m/z found, 244.1 [M+H]⁺. ¹H NMR (400 MHZ, DMSO-d₆) δ 8.23 (d, J=4.4 Hz, 1H), 6.88 (s, 1H), 4.99-4.95 (m, 1H), 4.80 (t, J=6.8 Hz, 2H), 4.61 (t, J=6.4 Hz, 2H), 2.41 (s, 3H).

### Step B: 6-chloro-4-methyl-N₂-(oxetan-3-yl)pyridine-2,3-diamine

To a solution of 6-chloro-4-methyl-3-nitro-N-(oxetan-3-yl) pyridin-2-amine (1.56 g, 6.40 mmol, 1.0 eq) and Ammonium chloride (1.71 g, 32.0 mmol, 5.0 eq) in THF (30.0 mL), EtOH (30.0 mL), and Water (15.0 mL) was added Iron (1.79 g, 32.0 mmol, 5.0 eq) at room temperature. The mixture was stirred at 70° C. for 1 h. After cooled to room temperature, the mixture was filtered and the cake was washed with EA (100 mL×3). The combined filtrates were concentrated under reduced pressure and the residue was diluted with EA (60 mL). The organic layer was washed with water (100 mL) and brine (100 mL), dried over anhydrous Na₂SO₄, filtered and concentrated under reduced pressure to give 6-chloro-4-methyl-N₂-(oxetan-3-yl)pyridine-2,3-diamine (1.20 g, yield 88%) as a yellow solid. LC-MS (ESI): mass calced for: C₉H₁₂ClN₃O, 213.07; m/z found, 214.1 [M+H]⁺.

### Step C: N-(6-chloro-4-methyl-2-(oxetan-3-ylamino)pyridin-3-yl) acetamide

To a solution of 6-chloro-4-methyl-N₂-(oxetan-3-yl)pyridine-2,3-diamine (1.10 g, 5.15 mmol, 1.0 eq) in DMF (20.0 mL) were added Triethylamine (1.56 g, 2.15 mL, 15.4 mmol, 3.0 eq) and Ac₂O (1.58 g, 1.46 mL, 15.4 mmol, 3.0 eq) under N₂ at 25° C. The mixture was stirred at 80° C. for 16 h. After cooled to room temperature, the reaction mixture was diluted with H₂O (30 mL) and extracted with EtOAc (20 mL×3). The organic layer was washed with brine (20 mL), dried over anhydrous Na₂SO₄, filtered and concentrated under reduced pressure. The residue was purified with flash column chromatography on silica gel (EA in PE, from 0% to 20% v/v) to afford N-(6-chloro-4-methyl-2-(oxetan-3-ylamino)pyridin-3-yl) acetamide (0.77 g, yield 58%) as a yellow solid. LC-MS (ESI): mass calced for: C₁₁H₁₄ClN₃O₂, 255.08; m/z found, 256.3 [M+H]⁺.

### Step D: 5-chloro-2,7-dimethyl-3-(oxetan-3-yl)-3H-imidazo[4,5-b]pyridine

To a solution of N-(6-chloro-4-methyl-2-(oxetan-3-ylamino)pyridin-3-yl) acetamide (1.45 g, 5.67 mmol, 1.0 eq) in EtOH (50.0 mL) was added NaOH (2.27 g, 56.7 mmol, 10.0 eq) under N₂ at 25° C. and the mixture was stirred at 75° C. for 5 h. After evaporation, the residue was diluted with H₂O (40 mL) and extracted with EA (50 mL×3). The organic layer was washed with brine (40 mL×2), dried over anhydrous sodium sulfate, filtered and concentrated under reduced pressure. The residue was purified with flash column chromatography on silica gel (MEOH in DCM, from 0% to 5% v/v) to afford 5-chloro-2,7-dimethyl-3-(oxetan-3-yl)-3H-imidazo[4,5-b]pyridine (1.20 g, yield 89%) as a yellow solid. LC-MS (ESI): mass calced for: C₁₁H₁₂ClN₃O, 237.07; m/z found, 238.2 [M+H]⁺. ¹H NMR (400 MHZ, DMSO-d₆) δ 7.18 (s, 1H), 5.78-5.70 (m, 1H), 5.34 (t, J=6.8 Hz, 2H), 4.93-4.87 (m, 2H), 2.56 (s, 3H), 2.51 (s, 3H).

### Step E: 7-(bromomethyl)-5-chloro-2-methyl-3-(oxetan-3-yl)-3H-imidazo[4,5-b]pyridine (100a) and 2-(bromomethyl)-5-chloro-7-methyl-3-(oxetan-3-yl)-3H-imidazo[4,5-b]pyridine (100b)

To a suspension of 5-chloro-2,7-dimethyl-3-(oxetan-3-yl)-3H-imidazo[4,5-b]pyridine (100 mg, 421 μmol, 1.0 eq) and N-bromosuccinimide (112 mg, 631 μmol, 1.5 eq) in CCl₄ (10.0 mL) was added Benzoyl peroxide (15.3 mg, 63.1 μmol, 0.15 eq) at room temperature. The reaction mixture was stirred at 90° C. for 5 h. After evaporation, the mixture was diluted with DCM (50 mL), washed with saturated aqueous NaHCO₃ solution (30 mL×2) and brine (30 mL). The organic layer was dried over anhydrous Na₂SO₄, filtered and concentrated under reduced pressure. The crude product was purified by Prep-TLC(DCM/MeOH=20/1 v/v) to give 7-(bromomethyl)-5-chloro-2-methyl-3-(oxetan-3-yl)-3H-imidazo[4,5-b]pyridine (100a) (4.00 mg, yield 3%) as a yellow solid and 2-(bromomethyl)-5-chloro-7-methyl-3-(oxetan-3-yl)-3H-imidazo[4,5-b]pyridine (100b) (30.0 mg, yield 22%) as a yellow solid.

7-(bromomethyl)-5-chloro-2-methyl-3-(oxetan-3-yl)-3H-imidazo[4,5-b]pyridine (100a): LC-MS (ESI): mass calced for: C₁₁H₁₁BrClN₃O, 314.98; m/z found, 315.99 [M+H]⁺. ¹H NMR (400 MHZ, DMSO-d₆) δ 7.45 (s, 1H), 5.81-5.73 (m, 1H), 5.35 (t, J=6.8 Hz, 2H), 4.92 (d, J=7.0 Hz, 2H), 4.89 (s, 2H), 2.62 (s, 3H).

2-(bromomethyl)-5-chloro-7-methyl-3-(oxetan-3-yl)-3H-imidazo[4,5-b]pyridine (100b): LC-MS (ESI): mass calced for: C₁₁H₁₁BrClN₃O, 314.98; m/z found, 315.99 [M+H]⁺. ¹H NMR (400 MHZ, DMSO-d₆) δ 7.32 (s, 1H), 5.95-5.87 (m, 1H), 5.50-5.43 (m, 2H), 4.97 (s, 2H), 4.92-4.88 (m, 2H), 2.56 (s, 3H).

### Intermediate 101:6-chloro-1-(1-methylazetidin-3-yl)-4-(pyrrolidin-1-ylmethyl)-1H-pyrrolo[2,3-b]pyridine

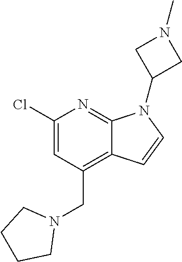 [SMILES: CN1CC(n2ccc3c(CN4CCCC4)cc(Cl)nc32)C1]

To a solution of 1-(azetidin-3-yl)-6-chloro-4-(pyrrolidin-1-ylmethyl)-1H-pyrrolo[2,3-b]pyridine (100 mg, 344 μmol, 1.0 eq) in DCM (5.0 mL) was added formaldehyde (33% wt in water) (271 μL, 3.4 mmol, 10.0 eq) at 25° C. The reaction mixture was stirred at 25° C. for 15 h. NaBH(OAc) 3 (109 mg, 516 μmol, 1.5 eq) was added to above mixture and the reaction mixture was stirred at 25° C. for 1 h. The reaction mixture was filtered and concentrated under reduced pressure. The residue was purified by Prep-TLC(DCM/MeOH=5/1 v/v) to give 6-chloro-1-(1-methylazetidin-3-yl)-4-(pyrrolidin-1-ylmethyl)-1H-pyrrolo[2,3-b]pyridine (80 mg, yield 76%) as a yellow solid. LC-MS (ESI): mass calcd. for C₁₆H₂₁ClN₄, 304.2; m/z found, 305.2 [M+H]⁺.

### Intermediate 102:6-chloro-1-(1-methylazetidin-3-yl)-4-(pyrrolidin-1-ylmethyl)-1H-pyrazolo[3,4-b]pyridine

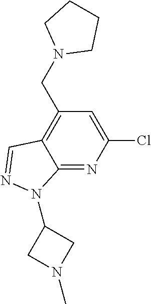 [SMILES: CN1CC(n2ncc3c(CN4CCCC4)cc(Cl)nc32)C1]

### Step A: tert-butyl 3-(6-chloro-4-(pyrrolidin-1-ylmethyl)-2H-pyrazolo[3,4-b]pyridin-2-yl) azetidine-1-carboxylate

To a solution of 6-chloro-4-(pyrrolidin-1-ylmethyl)-1H-pyrazolo[3,4-b]pyridine (120 mg, 507 μmol, 1.0 eq) and tert-butyl 3-bromoazetidine-1-carboxylate (180 mg, 760 μmol, 1.5 eq) in DMF (3.00 mL) were added potassium carbonate (210 mg, 1.52 mmol, 3.0 eq) and the mixture was stirred at 80° C. for 16 h. After cooled to room temperature, the residue was poured into water (10 mL) and extracted with EtOAc (10 mL×3). The organic layer was washed with brine (10 mL×4), dried over MgSO₄, filtered and concentrated under reduced pressure. The residue was purified by Prep-TLC(PE/EA=1/1 v/v) to obtain tert-butyl 3-(6-chloro-4-(pyrrolidin-1-ylmethyl)-1H-pyrazolo[3,4-b]pyridin-1-yl) azetidine-1-carboxylate (70.0 mg, yield 32%) as a yellow oil. LC-MS (ESI): mass calced for: C₁₉H₂₆ClN₅O₂, 391.90; m/z found, 392.2 [M+H]⁺. ¹H NMR (400 MHZ, DMSO-d₆) δ 8.45 (s, 1H), 7.26 (s, 1H), 5.72-5.68 (m, 1H), 4.31 (t, J=7.6 Hz, 2H), 4.24 (t, J=7.6 Hz, 2H), 3.97 (s, 2H), 2.51 (s, 4H), 1.74 (s, 4H), 1.42 (s, 9H).

### Step B: 1-(azetidin-3-yl)-6-chloro-4-(pyrrolidin-1-ylmethyl)-1H-pyrazolo[3,4-b]pyridine

To a solution of tert-butyl 3-(6-chloro-4-(pyrrolidin-1-ylmethyl)-1H-pyrazolo[3,4-b]pyridin-1-yl) azetidine-1-carboxylate (70.0 mg, 179 μmol, 1.0 eq) in 1,4-Dioxane (2.00 mL) was added hydrogen chloride (4 M in dioxane) (0.4 mL, 1.6 mmol, 9.0 eq) and the mixture was stirred at 25° C. for 16 h. The reaction mixture was diluted with EtOAc (30 mL), washed with saturated aqueous NaHCO₃ solution (30 mL×3) and brine (30 mL), dried over MgSO₄, filtered and concentrated under reduced pressure. The residue was purified by Prep-TLC (DCM/MeOH=10/1 v/v) to obtain 1-(azetidin-3-yl)-6-chloro-4-(pyrrolidin-1-ylmethyl)-1H-pyrazolo[3,4-b]pyridine (40.0 mg, yield 69%) as a yellow oil. LC-MS (ESI): mass calced for: C₁₄H₁₈ClN₅, 291.13; m/z found, 292.2 [M+H]⁺.

### Step C: 6-chloro-1-(1-methylazetidin-3-yl)-4-(pyrrolidin-1-ylmethyl)-1H-pyrazolo[3,4-b]pyridine

To a solution of 1-(azetidin-3-yl)-6-chloro-4-(pyrrolidin-1-ylmethyl)-1H-pyrazolo[3,4-b]pyridine (40.0 mg, 137 μmol, 1.0 eq) and Formaldehyde (30% W.t in H₂O) (6.18 mg, 5.67 μL, 206 μmol, 1.5 eq) in MeOH (2.00 mL) was added AcOH (0.1 mL) and the mixture was stirred at room temperature for 1 h. Then Sodium triacetoxyborohydride (72.6 mg, 50.8 μL, 343 μmol, 2.5 eq) was added to above mixture and the mixture was stirred at room temperature overnight. The mixture was poured into water (6 mL) and extracted with DCM (10 mL×3). The organic layer was dried over MgSO₄, filtered and concentrated under reduced pressure. The residue was purified by Prep-TLC(DCM/MeOH=8/1 v/v) to give 6-chloro-1-(1-methylazetidin-3-yl)-4-(pyrrolidin-1-ylmethyl)-1H-pyrazolo[3,4-b]pyridine (30.0 mg, yield 64%) as a yellow oil. LC-MS (ESI): mass calced for: C₁₅H₂OClN₅, 305.14; m/z found, 306.1 [M+H].

### Intermediate 103:6-chloro-5-ethyl-1-(oxetan-3-yl)-1H-pyrrolo[2,3-b]pyridine-4-carbaldehyde

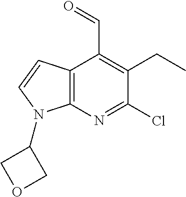 [SMILES: CCc1c(Cl)nc2c(ccn2C2COC2)c1C=O]

### Step A: methyl 2-amino-5-bromo-3-iodoisonicotinate

To a stirred solution of methyl 2-amino-5-bromoisonicotinate (5.0 g, 21.6 mmol, 1.0 eq) in ACN (100.0 mL) and TFA (10.0 mL) was added NIS (7.30 g, 32.5 mmol, 1.5 eq). The reaction mixture was stirred under nitrogen atmosphere at 70° C. for 16 h. After cooled to room temperature, the mixture was diluted with H₂O (100 mL) and extracted with EtOAc (100 mL×3). The combined organic layers were washed with saturated aqueous Na₂S₂O₃ solution (100 mL×3) and brine (100 mL), dried over anhydrous Na₂SO₄, filtered and concentrated in vacuum. The residue was purified by flash column chromatography on silica gel (PE/EtOAc=1/1 v/v) to give methyl 2-amino-5-bromo-3-iodoisonicotinate (7.0 g, yield 90%) as a light yellow solid. LC-MS (ESI): mass calced for: C₇H₆BrIN₂O₂, 355.87; m/z found, 356.9 [M+H]⁺.

### Step B: methyl 5-bromo-1H-pyrrolo[2,3-b]pyridine-4-carboxylate

To a stirred solution of methyl 2-amino-5-bromo-3-iodoisonicotinate (7.0 g, 19.6 mmol, 1.0 eq) and (E)-2-(2-ethoxyvinyl)-4,4,5,5-tetramethyl-1,3,2-dioxaborolane (7.77 g, 39.2 mmol, 2.0 eq) in ACN (100 mL) and H₂O (10.0 mL) were added 2-Dicyclohexylphosphino-2,6-Dimethoxy-1,1-Biphenyl (805 mg, 1.96 mmol, 0.1 eq), Pd (OAc)₂ (220 mg, 981 μmol, 0.05 eq), and Potassium phosphate tribasic (8.33 g, 39.2 mmol, 2.0 eq). The reaction mixture was stirred under nitrogen atmosphere at 90° C. for 16 h. After cooled to room temperature, The mixture was diluted with H₂O (150 mL) and extracted with EtOAc (200 mL×3). The combined organic layers were washed with brine (200 mL), dried over anhydrous Na₂SO₄, filtered and concentrated in vacuum. The residue was purified by flash column chromatography on silica gel (PE/EtOAc=3/1 v/v) to give methyl (E)-2-amino-5-bromo-3-(2-ethoxyvinyl) isonicotinate (5.00 g, 16.6 mmol, 84.7%) as brown oil.

A solution of methyl (E)-2-amino-5-bromo-3-(2-ethoxyvinyl) isonicotinate (5.0 g, 16.6 mmol, 1.0 eq) in AcOH (60.0 mL) was stirred under N₂ at 110° C. for 16 h. The reaction mixture was cooled to room temperature and concentrated under reduced pressure. The mixture was quenched with ice-water (30 mL), adjusted to pH 7-8 with saturated aqueous NaHCO₃ solution, and extracted with EtOAc (150 mL×3). The combined organic layers were dried over anhydrous Na₂SO₄, filtered and concentrated in vacuum. The residue was purified by flash column chromatography on silica gel (PE/EtOAc=1/1 v/v) to give methyl 5-bromo-1H-pyrrolo[2,3-b]pyridine-4-carboxylate (2.60 g, yield 61%) as a yellow solid. LC-MS (ESI): mass calced for: C₉H₇BrN₂O₂, 253.97; m/z found, 255.0 [M+H]⁺.

### Step C: methyl 5-vinyl-1H-pyrrolo[2,3-b]pyridine-4-carboxylate

To a stirred solution of methyl 5-bromo-1H-pyrrolo[2,3-b]pyridine-4-carboxylate (2.50 g, 9.80 mmol, 1.0 eq) and Potassium vinyltrifluoroborate (1.97 g, 14.7 mmol, 1.5 eq) in 1,4-Dioxane (30.0 mL) and H₂O (3.0 mL) were added Na₂CO₃ (3.12 g, 29.4 mmol, 3.0 eq) and Pd (PPh₃)₄ (1.13 g, 980 μmol, 0.1 eq). The reaction mixture was stirred under N₂ at 95° C. for 16 h. After cooled to room temperature, the mixture was diluted with water (30 mL) and extracted with EtOAc (50 mL×3). The combined organic layers were washed with brine (100 mL), dried over anhydrous Na₂SO₄, filtered and concentrated under reduced pressure. The residue was purified by flash column chromatography on silica gel (PE/EtOAc=3/1 v/v) to give methyl 5-vinyl-1H-pyrrolo[2,3-b]pyridine-4-carboxylate (950 mg, yield 48%) as a yellow solid. LC-MS (ESI): mass calced for: C₁₁H₁₀N₂O₂, 202.07; m/z found, 203.1 [M+H]⁺.

### Step D: methyl 5-ethyl-1H-pyrrolo[2,3-b]pyridine-4-carboxylate

To a stirred solution of methyl 5-vinyl-1H-pyrrolo[2,3-b]pyridine-4-carboxylate (900 mg, 4.45 mmol, 1.0 eq) in MeOH (20.0 mL) was added 10% Pd/C(900 mg, 8.46 mmol, 1.9 eq). The reaction mixture was stirred under H₂ (1atm) at 25° C. for 2 h. After filtered, the filtrate was concentrated to afford methyl 5-ethyl-1H-pyrrolo[2,3-b]pyridine-4-carboxylate (850 mg, yield 94%) as a white solid. LC-MS (ESI): mass calced for: C₁₁H₁₂N₂O₂, 204.09; m/z found, 205.1 [M+H]⁺. ¹H NMR (400 MHZ, DMSO-d₆) δ 11.85 (s, 1H), 8.22 (s, 1H), 7.60-7.58 (m, 1H), 6.57 (s, 1H), 3.95 (s, 3H), 2.90 (q, J=7.2 Hz, 2H), 1.18 (t, J=7.2 Hz, 3H).

### Step E: methyl 6-chloro-5-ethyl-1H-pyrrolo[2,3-b]pyridine-4-carboxylate

To a stirred solution of methyl 5-ethyl-1H-pyrrolo[2,3-b]pyridine-4-carboxylate (850 mg, 4.16 mmol, 1.0 eq) in EA (25.0 mL) was added m-CPBA (1.08 g, 6.24 mmol, 1.5 eq). The reaction mixture was stirred at 25° C. for 16 h. After filtered, the filter cake was washed with EA and dried to afford 5-ethyl-4-(methoxycarbonyl)-1H-pyrrolo[2,3-b]pyridine 7-oxide (350 mg, 1.59 mmol, yield 38%) as a white solid.

A solution of 5-ethyl-4-(methoxycarbonyl)-1H-pyrrolo[2,3-b]pyridine 7-oxide (350 mg, 1.59 mmol, 1.0 eq) in DMF (10.0 mL) was stirred under nitrogen atmosphere at 50° C. for 10 min. And MsCl (455 mg, 310 μL, 3.97 mmol, 2.5 eq) was added dropwise to above mixture and the resulting reaction mixture was stirred under nitrogen atmosphere at 80° C. for 30 min. After cooled to room temperature, the reaction mixture was diluted with EA (40 mL), adjusted to pH 7-8 with saturated aqueous NaHCO₃ solution (30 mL), and extracted with EtOAc (20 mL×3). The combined organic layers were washed with brine (50 mL), dried over anhydrous Na₂SO₄, filtered and concentrated under reduced pressure. The residue was purified by flash column chromatography on silica gel (PE/EtOAc=1/1 v/v) to give methyl 6-chloro-5-ethyl-1H-pyrrolo[2,3-b]pyridine-4-carboxylate (250 mg, yield 66%) as a white solid. LC-MS (ESI): mass calced for: C₁₁H₁₁ClN₂O₂, 238.05; m/z found, 239.1 [M+H]⁺.

### Step F: methyl 6-chloro-5-ethyl-1-(oxetan-3-yl)-1H-pyrrolo[2,3-b]pyridine-4-carboxylate

To a stirred mixture of methyl 6-chloro-5-ethyl-1H-pyrrolo[2,3-b]pyridine-4-carboxylate (220 mg, 922 μmol, 1.0 eq) and 3-iodooxetane (254 mg, 1.38 mmol, 1.5 eq) in DMF (8.0 mL) was added Cs₂CO₃ (901 mg, 2.77 mmol, 3.0 eq). The resulting mixture was stirred at 80° C. for 2 h. After cooled to room temperature, the reaction mixture was diluted with H₂O (50 mL) and extracted with EtOAc (50 mL×3). The combined organic layers were washed with brine (50 mL×3), dried over anhydrous Na₂SO₄, filtered and concentrated under reduced pressure to give methyl 6-chloro-5-ethyl-1-(oxetan-3-yl)-1H-pyrrolo[2,3-b]pyridine-4-carboxylate (250 mg, yield 92%) as a light yellow solid. LC-MS (ESI): mass calced for: C₁₄H₁₅ClN₂O₃ 294.08; m/z found, 295.1 [M+H]⁺.

### Step G: (6-chloro-5-ethyl-1-(oxetan-3-yl)-1H-pyrrolo[2,3-b]pyridin-4-yl) methanol

To a stirred mixture of methyl 6-chloro-5-ethyl-1-(oxetan-3-yl)-1H-pyrrolo[2,3-b]pyridine-4-carboxylate (250 mg, 848 μmol, 1.0 eq) in THF (10.0 mL) was added LiAlH₄ (48.3 mg, 1.27 mmol, 1.5 eq). The resulting mixture was stirred at 25° C. for 1 h. The reaction was quenched with sodium sulfate decahydrate and filtered. The filtrate was concentrated under reduced pressure to obtain (6-chloro-5-ethyl-1-(oxetan-3-yl)-1H-pyrrolo[2,3-b]pyridin-4-yl) methanol (200 mg, yield 88%) as a light yellow solid. LC-MS (ESI): mass calced for: C₁₃H₁₅ClN₂O₂, 266.08; m/z found, 267.1 [M+H]⁺

### Step H: 6-chloro-5-ethyl-1-(oxetan-3-yl)-1H-pyrrolo[2,3-b]pyridine-4-carbaldehyde

To a stirred mixture of (6-chloro-5-ethyl-1-(oxetan-3-yl)-1H-pyrrolo[2,3-b]pyridin-4-yl) methanol (200 mg, 750 μmol, 1.0 eq) in DMSO (8.0 mL) was added IBX (315 mg, 1.12 mmol, 1.5 eq). The resulting mixture was stirred at 25° C. for 1 h. The reaction mixture was quenched with saturated aqueous Na₂SO₃ solution (40 mL) and extracted with EtOAc (50 mL×3). The combined organic layers were washed with brine (50 mL×4), dried over anhydrous Na₂SO₄, filtered and concentrated in vacuum. The residue was purified by flash column chromatography on silica gel (PE/EtOAc=3/1 v/v) to give 6-chloro-5-ethyl-1-(oxetan-3-yl)-1H-pyrrolo[2,3-b]pyridine-4-carbaldehyde (110 mg, yield 55%) as a yellow solid. LC-MS (ESI): mass calced for: C₁₃H₁₃ClN₂O₂, 264.07; m/z found, 265.1 [M+H]⁺.

### Intermediate 104:5,6-dichloro-1-(oxetan-3-yl)-4-(pyrrolidin-1-ylmethyl)-1H-pyrrolo[2,3-b]pyridine

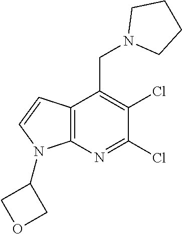 [SMILES: Clc1nc2c(ccn2C2COC2)c(CN2CCCC2)c1Cl]

To a solution of 5,6-dichloro-4-(pyrrolidin-1-ylmethyl)-1H-pyrrolo[2,3-b]pyridine (70.0 mg, 259 μmol, 1.0 eq) in DMF (2.00 mL) were added Cs₂CO₃ (253 mg, 777 μmol, 3.0 eq) and 3-iodooxetane (119 mg, 648 μmol, 2.5 eq). The reaction mixture was stirred at 80° C. for 3 h. After cooled to room temperature, the mixture was quenched with water (10.0 mL) and extracted with DCM (6.0 mL×3). The combined organic layers were washed with brine (15 mL×4), dried over anhydrous Na₂SO₄, filtered and concentrated under reduced pressure. The crude product was purified by Prep-TLC(DCM/MeOH=10/1 v/v) to afford 5,6-dichloro-1-(oxetan-3-yl)-4-(pyrrolidin-1-ylmethyl)-1H-pyrrolo[2,3-b]pyridine (45.0 mg, yield 53%) as a yellow oily. LC-MS (ESI): mass calced for: C₁₅H₁₇C₁₂N₃O, 326.22; m/z found, 326.2 [M+H]⁺.

### Intermediate 105:6-chloro-1-(3,3-difluorocyclobutyl)-1H-pyrrolo[2,3-b]pyridine-4-carbaldehyde

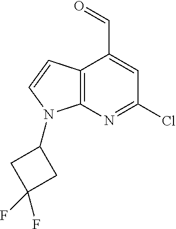 [SMILES: O=Cc1cc(Cl)nc2c1ccn2C1CC(F)(F)C1]

### Step A: methyl 6-chloro-1-(5,8-dioxaspiro[3.4]octan-2-yl)-1H-pyrrolo[2,3-b]pyridine-4-carboxylate

To a solution of methyl 6-chloro-1H-pyrrolo[2,3-b]pyridine-4-carboxylate (2.0 g, 9.5 mmol, 1.0 eq) in DMF (25.0 mL) were added Cs₂CO₃ (9.3 g, 28.5 mmol, 3.0 eq) and 2-bromo-5,8-dioxaspiro[3.4]octane (1.8 g, 9.5 mmol, 1.0 eq) at 25° C. The reaction mixture was stirred at 80° C. for 6 h. After cooled to room temperature, the mixture was quenched with H₂O (100 mL) and extracted with EA (100 mL×3). The organic layer was washed with brine (100 mL×4), dried over anhydrous Na₂SO₄, filtered and concentrated under reduced pressure. The residue was purified by flash column chromatography on silica gel (PE/EA=10/1 v/v) to give methyl 6-chloro-1-(5,8-dioxaspiro[3.4]octan-2-yl)-1H-pyrrolo[2,3-b]pyridine-4carboxylate (1.0 g, yield 33%) as a brown solid. LC-MS (ESI): mass calced for: C₁₅H₁₅ClN₂O₄, 322.1; m/z found, 323.1 [M+H]⁺.

### Step B: methyl 6-chloro-1-(3-oxocyclobutyl)-1H-pyrrolo[2,3-b]pyridine-4-carboxylate

To a solution of methyl 6-chloro-1-(5,8-dioxaspiro[3.4]octan-2-yl)-1H-pyrrolo[2,3-b]pyridine-4-carboxylate (1.0 g, 3.1 mmol, 1.0 eq) in THF (5 mL) was added aqueous HCl solution (2 M) (10 mL) at 25° C. The reaction mixture was stirred at 60° C. for 1 h. After cooled to room temperature, the reaction mixture was quenched with water (10 mL) and extracted with EA (10 mL×3). The organic layer was washed with brine (10 mL), dried over anhydrous Na₂SO₄, and filtered. The filtrate was concentrated under reduced pressure to afford methyl 6-chloro-1-(3-oxocyclobutyl)-1H-pyrrolo[2,3-b]pyridine-4-carboxylate (640 mg, yield 74%) as a yellow solid. The crude product was directly used in next step without further purification. LC-MS (ESI): mass calced for: C₁₃H₁₁ClN₂O₃, 278.1; m/z found, 279.1 [M+H]⁺. ¹H NMR (400 MHz, DMSO-d₆) δ 8.19 (s, 1H), 7.62 (s, 1H), 6.97 (s, 1H), 5.60-5.52 (m, 1H), 3.97 (s, 3H), 3.76-3.60 (m, 4H).

### Step C: methyl 6-chloro-1-(3,3-difluorocyclobutyl)-1H-pyrrolo[2,3-b]pyridine-4-carboxylate

To a solution of methyl 6-chloro-1-(3-oxocyclobutyl)-1H-pyrrolo[2,3-b]pyridine-4-carboxylate (235 mg, 843 μmol, 1.0 eq) in DCM (20.0 mL) was dropwise added DAST (816 mg, 653 μL, 5.06 mmol, 6.0 eq) under nitrogen at 0° C. . . . The reaction mixture was stirred at 25° C. for 16 h. The reaction mixture was quenched with saturated aqueous NaHCO₃ solution (10 mL) and extracted with DCM (10 mL×3). The organic layer was washed with brine (20 mL), dried over anhydrous Na₂SO₄, filtered and concentrated under reduced pressure. The residue was purified by flash column chromatography on silica gel (PE/EA=10/1 v/v) to provide methyl 6-chloro-1-(3,3-difluorocyclobutyl)-1H-pyrrolo[2,3-b]pyridine-4-carboxylate (65.0 mg, yield 25%) as a yellow solid. LC-MS (ESI): mass calced for: C₁₃H₁₁CIF2N₂O₂, 300.05; m/z found, 301.0 [M+H]⁺

### Step D: (6-chloro-1-(3,3-difluorocyclobutyl)-1H-pyrrolo[2,3-b]pyridin-4-yl) methanol

To a solution of methyl 6-chloro-1-(3,3-difluorocyclobutyl)-1H-pyrrolo[2,3-b]pyridine-4-carboxylate (144 mg, 479 μmol, 1.0 eq) in THF (10.0 mL) was dropwise added Aluminum lithium hydride (2.5 M in THF) (383 μL, 958 μmol, 2.0 eq) at 0° C. The reaction mixture was stirred at 0° C. for 20 min. The reaction mixture was quenched with Na₂SO₄·10H₂O, and stirred for 30 min and filtered. The filtrate was diluted with water (10 mL) and extracted with ethyl acetate (15 mL×3). The combined organic extracts were washed with water (15 mL) and brine (15 mL), dried over anhydrous sodium sulfate, and filtered. The filtrate was concentrated under reduced pressure to give (6-chloro-1-(3,3-difluorocyclobutyl)-1H-pyrrolo[2,3-b]pyridin-4-yl) methanol (120 mg, yield 92%) as a yellow solid. The crude product was directly used in next step without further purification. LC-MS (ESI): mass calced for: C₁₂H₁₁CIF2N₂O, 272.05; m/z found, 273.1 [M+H]⁺.

### Step E: 6-chloro-1-(3,3-difluorocyclobutyl)-1H-pyrrolo[2,3-b]pyridine-4-carbaldehyde

To a solution of (6-chloro-1-(3,3-difluorocyclobutyl)-1H-pyrrolo[2,3-b]pyridin-4-yl) methanol (120 mg, 440 μmol, 1.0 eq) in DMSO (8.00 mL) was added IBX (Purity 45% W.t) (308 mg, 1.10 mmol, 2.5 eq) at room temperature. The reaction mixture was stirred at 25° C. for 1 h. The reaction mixture was diluted with water (20 mL) and extracted with ethyl acetate (20 mL×3). The combined organic extracts were washed with saturated aqueous Na₂S₂O₃ solution (20 mL×2), saturated aqueous NaHCO₃ solution (20 mL) and brine (20 mL), dried over anhydrous sodium sulfate, filtered and concentrated under reduced pressure. The residue was purified by Prep-TLC(EA/PE=1/5 v/v) to provide 6-chloro-1-(3,3-difluorocyclobutyl)-1H-pyrrolo[2,3-b]pyridine-4-carbaldehyde (50.0 mg, yield 42%) as a yellow solid. LC-MS (ESI): mass calced for: C₁₂H₉CIF2N₂O, 270.04; m/z found, 271.1 [M+H]⁺. ¹H NMR (400 MHZ, DMSO-d₆) δ 10.30 (s, 1H), 8.11 (s, 1H), 7.78 (s, 1H), 7.09 (s, 1H), 5.25-5.20 (m, 1H), 3.29-3.23 (m, 4H).

### Intermediate 106:6-chloro-2-methyl-1-(oxetan-3-yl)-1H-pyrrolo[2,3-b]pyridine-4-carbaldehyde

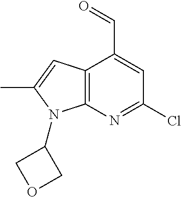 [SMILES: Cc1cc2c(C=O)cc(Cl)nc2n1C1COC1]

### Step A: 4-bromo-2-methyl-1H-pyrrolo[2,3-b]pyridine 7-oxide

To a solution of 4-bromo-2-methyl-1H-pyrrolo[2,3-b]pyridine (4.00 g, 19.0 mmol, 1.0 eq) in tert-butyl methyl ether (50.0 mL) was added m-CPBA (4.74 g, 27.5 mmol, 1.45 eq) at 0° C. The mixture was stirred at 25° C. for 16 h. The mixture was quenched with saturated aqueous NaHCO₃ solution (30 mL) and filtered. The filter cake was washed with water (20 mL) and EA (20 mL×3), and dried to give 4-bromo-2-methyl-1H-pyrrolo[2,3-b]pyridine 7-oxide (2.70 g, yield 90%) as a gray solid. LC-MS (ESI): mass calced for: C₈H₇BrN₂O, 227.06; m/z found, 227.1 [M+H]⁺.

### Step B: 4-bromo-6-chloro-2-methyl-1H-pyrrolo[2,3-b]pyridine

To a solution of 4-bromo-2-methyl-1H-pyrrolo[2,3-b]pyridine 7-oxide (2.70 g, 11.9 mmol, 1.0 eq) in Toluene (100 mL) was added POCl₃ (50.0 mL). The mixture was stirred at 110° C. for 24 h. After cooled to room temperature, the reaction mixture concentrated in vacuum. The residue was diluted with EA (80 mL), washed with aqueous NaHCO₃ solution (80 mL) and brine (80 mL), dried over anhydrous Na₂SO₄, and filtered. The filtrate was concentrated under reduced pressure to give crude product 4-bromo-6-chloro-2-methyl-1H-pyrrolo[2,3-b]pyridine (2.30 g, yield 79%) as a brown solid. LC-MS (ESI): mass calced for: C₈H₆BrClN₂, 245.5; m/z found, 245.3 [M+H]⁺.

### Step C: 4-bromo-6-chloro-2-methyl-1-(oxetan-3-yl)-1H-pyrrolo[2,3-b]pyridine

To a solution of 4-bromo-6-chloro-2-methyl-1H-pyrrolo[2,3-b]pyridine (2.30 g, 9.37 mmol, 1.0 eq) in DMF (35.00 mL) were added Cs₂CO₃ (9.16 g, 28.1 mmol, 3.0 eq) and 3-iodooxetane (5.17 g, 28.1 mmol, 3.0 eq). The reaction mixture was stirred at 80° C. for 6 h. After cooled to room temperature, the mixture quenched with saturated aqueous NH₄Cl solution (100 mL) and extracted with EA (50 mL×3). The organic layer was washed with brine (40 mL×4), dried over anhydrous Na₂SO₄, filtered and concentrated under reduced pressure. The crude product was purified by flash column chromatography on silica gel (ethyl acetate in petroleum ether, 15% v/v) to obtain 4-bromo-6-chloro-2-methyl-1-(oxetan-3-yl)-1H-pyrrolo[2,3-b]pyridine (850 mg, yield 30%) as a yellow solid. LC-MS (ESI): mass calced for: C₁₁H₁₀BrClN₂O, 301.57; m/z found, 303.2 [M+H]⁺. ¹H NMR (400 MHZ, DMSO-d₆) δ 7.49 (s, 1H), 6.33 (s, 1H), 5.80-5.69 (m, 1H), 5.33 (t, J=6.4 Hz, 2H), 4.92 (t, J=7.0 Hz, 2H), 2.49 (s, 3H).

### Step D: 6-chloro-2-methyl-1-(oxetan-3-yl)-4-vinyl-1H-pyrrolo[2,3-b]pyridine

To a solution of 4-bromo-6-chloro-2-methyl-1-(oxetan-3-yl)-1H-pyrrolo[2,3-b]pyridine (100.0 mg, 331.6 μmol, 1.0 eq), Potassium vinyl trifluoroborate (48.86 mg, 364.8 μmol, 1.1 eq), and Potassium carbonate (137.5 mg, 994.8 μmol, 3.0 eq) in 1,4-Dioxane (3.00 mL) and water (0.3 mL) was added Pd(Ph₃P)₄ (38.32 mg, 33.16 μmol, 0.1 eq). The mixture was stirred under N₂ at 95° C. for 3 h. After cooled to room temperature, the reaction mixture was filtered and the filtrate was concentrated in vacuum. The crude product was purified by Prep-TLC(PE/EA=6/1 v/v) to obtain 6-chloro-2-methyl-1-(oxetan-3-yl)-4-vinyl-1H-pyrrolo[2,3-b]pyridine (60.0 mg, yield 72%) as a brown oily. LC-MS (ESI): mass calced for: C₁₃H₁₃ClN₂O, 248.71; m/z found, 249.1 [M+H]⁺.

### Step E: 1-(6-chloro-2-methyl-1-(oxetan-3-yl)-1H-pyrrolo[2,3-b]pyridin-4-yl) ethane-1,2-diol

To a solution of 6-chloro-2-methyl-1-(oxetan-3-yl)-4-vinyl-1H-pyrrolo[2,3-b]pyridine (60.0 mg, 241 μmol, 1.0 eq) in Acetone (4.00 mL) and water (2.00 mL) were added NMO (56.5 mg, 482 μmol, 2.0 eq) and Potassiumosmate (VI) dihydrate (8.89 mg, 24.1 μmol, 0.1 eq). The reaction was stirred at 15° C. for 16 h. The reaction mixture was filtered and the filtrate was extracted with EA (5 mL×3). The organic layer was washed with brine (10 mL), dried over anhydrous Na₂SO₄, filtered and concentrated under reduced pressure to give crude product 1-(6-chloro-2-methyl-1-(oxetan-3-yl)-1H-pyrrolo[2,3-b]pyridin-4-yl) ethane-1,2-diol (68.0 mg, yield 99%) as a brown oily. LC-MS (ESI): mass calced for: C₁₃H₁₅ClN₂O₃, 282.72; m/z found, 283.2 [M+H]⁺.

### Step F: 6-chloro-2-methyl-1-(oxetan-3-yl)-1H-pyrrolo[2,3-b]pyridine-4-carbaldehyde

To a solution of 1-(6-chloro-2-methyl-1-(oxetan-3-yl)-1H-pyrrolo[2,3-b]pyridin-4-yl) ethane-1,2-diol (68.0 mg, 241 μmol, 1.0 eq) in THF (4.00 mL) and water (2.00 mL) was added Sodium metaperiodate (103 mg, 481 μmol, 2.0 eq) at 0° C. The reaction was stirred at 0° C. for 1 h. The mixture was quenched with saturated aqueous Na₂S₂O₃ solution (8 mL) and extracted with EA (5 mL×3). The organic layer was washed with brine (10 mL), dried over anhydrous Na₂SO₄, filtered and concentrated under reduced pressure. The crude product was purified by Prep-TLC(PE/EA=3/1 v/v) to obtain 6-chloro-2-methyl-1-(oxetan-3-yl)-1H-pyrrolo[2,3-b]pyridine-4-carbaldehyde (20.0 mg, yield 33%) as a yellow solid. LC-MS (ESI): mass calced for: C₁₂H₁₁ClN₂O₂, 250.68; m/z found, 251.0 [M+H]⁺.

### Intermediate 107:5,6-dichloro-1-methyl-1H-pyrrolo[2,3-b]pyridine-4-carbaldehyde

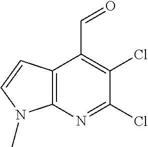

### Step A: 4-bromo-5-chloro-1-((2-(trimethylsilyl)ethoxy)methyl)-1H-pyrrolo[2,3-b]pyridine

To a solution of 4-bromo-5-chloro-1H-pyrrolo[2,3-b]pyridine (5.00 g, 21.6 mmol, 1.0 eq) in DMF (70.00 mL) was added NaH (60% suspend in oil) (1.04 g, 25.9 mmol, 1.2 eq) at 0° C. and the mixture was stirred at 0° C. for 30 min. SEM-C₁ (3.96 g, 23.8 mmol, 1.1 eq) was added to above mixture and the resulting reaction mixture was stirred at 25° C. for 1 h. The mixture was quenched with saturated aqueous NH₄Cl solution (100 mL) and extracted with EA (50 mL×3). The organic layer was washed with brine (100 mL×4), dried over anhydrous Na₂SO₄, filtered and concentrated under reduced pressure. The crude product was purified by flash column chromatography on silica gel (ethyl acetate in petroleum ether, 9% v/v) to give product 4-bromo-5-chloro-1-((2-(trimethylsilyl)ethoxy)methyl)-1H-pyrrolo[2,3-b]pyridine (5.20 g, yield 66%) as a colorless oil. LC-MS (ESI): mass calced for: C₁₃H₁₈BrClN₂OSi, 361.74; m/z found, 363.2 [M+H]⁺. ¹H NMR (400 MHZ, DMSO-d₆) δ 8.50 (s, 1H), 7.98 (d, J=2.2 Hz, 1H), 6.65 (d, J=2.2 Hz, 1H), 5.73 (s, 2H), 3.61 (t, J=7.8 Hz, 2H), 0.92 (t, J=7.8 Hz, 2H), −0.00 (s, 9H).

### Step B: methyl 5-chloro-1-((2-(trimethylsilyl)ethoxy)methyl)-1H-pyrrolo[2,3-b]pyridine-4-carboxylate

To a solution of 4-bromo-5-chloro-1-((2-(trimethylsilyl)ethoxy)methyl)-1H-pyrrolo[2,3-b]pyridine (5.20 g, 14.4 mmol, 1.0 eq) in MeOH (150 mL) were added TEA (7.27 g, 71.9 mmol, 5 eq) and [1,l′-Bis(diphenylphosphino) ferrocene]dichloropalladium (II) complex with dichloromethane (1.58 g, 2.16 mmol, 0.15 eq). The reaction mixture was stirred under CO atmosphere (1 atm) at 60° C. for 16 h. After cooled to room temperature, the reaction mixture was filtered and the filtrate was concentrated in vacuum. The crude product was purified by flash column chromatography on silica gel (ethyl acetate in petroleum ether, 9% v/v) to give product methyl 5-chloro-1-((2-(trimethylsilyl)ethoxy)methyl)-1H-pyrrolo[2,3-b]pyridine-4-carboxylate (4.50 g, yield 91%) as a colorless oil. LC-MS (ESI): mass calced for: C₁₅H₂₁ClN₂O₃Si, 340.88; m/z found, 341.2 [M+H]⁺. ¹H NMR (400 MHZ, DMSO-d₆) δ 8.54 (s, 1H), 8.01 (d, J=3.6 Hz, 1H), 6.79 (d, J=3.6 Hz, 1H), 5.76 (s, 2H), 4.10 (s, 3H), 3.65-3.58 (m, 2H), 0.95-0.88 (m, 2H),-0.01 (s, 9H).

### Step C: methyl 5-chloro-1-(hydroxymethyl)-1H-pyrrolo[2,3-b]pyridine-4-carboxylate

To a solution of methyl 5-chloro-1-((2-(trimethylsilyl)ethoxy)methyl)-1H-pyrrolo[2,3-b]pyridine-4-carboxylate (4.50 g, 13.2 mmol, 1.0 eq) in DCM (40.0 mL) was added TFA (4.0 mL). The reaction mixture was stirred at 20° C. for 2 h. The mixture was concentrated under reduced pressure to give crude product methyl 5-chloro-1-(hydroxymethyl)-1H-pyrrolo[2,3-b]pyridine-4-carboxylate (3.20 g, yield 99%) as a brown oil. LC-MS (ESI): mass calced for: C₁₀H₉ClN₂O₃, 240.64; m/z found, 241.1 [M+H]⁺.

### Step D: methyl 5-chloro-1H-pyrrolo[2,3-b]pyridine-4-carboxylate

To a solution of methyl 5-chloro-1-(hydroxymethyl)-1H-pyrrolo[2,3-b]pyridine-4-carboxylate (3.20 g, 13.3 mmol, 1.0 eq) in ACN (30.0 mL) and MeOH (10.0 mL) was added Ammonium hydroxide (10.0 mL). The reaction mixture was stirred at 20° C. for 30 min. After evaporation, the crude product was purified by flash column chromatography on silica gel (ethyl acetate in petroleum ether, 25% v/v) to give product methyl 5-chloro-1H-pyrrolo[2,3-b]pyridine-4-carboxylate (2.00 g, yield 71%) as a white solid. LC-MS (ESI): mass calced for: C₉H₇ClN₂O₂, 210.62; m/z found, 211.3 [M+H]⁺. ¹H NMR (400 MHZ, DMSO-d₆) δ 12.21 (s, 1H), 8.34 (s, 1H), 7.73 (d, J=3.4 Hz, 1H), 6.58 (d, J=3.4 Hz, 1H), 3.98 (s, 3H).

### Step E: 5-chloro-4-(methoxycarbonyl)-1H-pyrrolo[2,3-b]pyridine 7-oxide

To a solution of methyl 5-chloro-1H-pyrrolo[2,3-b]pyridine-4-carboxylate (1.00 g, 4.75 mmol, 1.0 eq) in Ethyl acetate (50.0 mL) was added m-CPBA (Purity 85%) (1.45 g, 7.12 mmol, 1.5 eq) at 0° C. and the mixture was stirred at 20° C. for 16 h. The mixture was quenched with saturated aqueous NaHCO₃ solution and filtered. The filter cake was washed with water and EA, and dried to give product 5-chloro-4-(methoxycarbonyl)-1H-pyrrolo[2,3-b]pyridine 7-oxide (490 mg, yield 45%) as a grey solid. LC-MS (ESI): mass calced for: C₉H₇ClN₂O₃, 226.62; m/z found, 227.3 [M+H]⁺. ¹H NMR (400 MHZ, DMSO-d₆) δ 12.98 (s, 1H), 8.48 (s, 1H), 7.65 (s, 1H), 6.76 (s, 1H), 3.96 (s, 3H).

### Step F: methyl 5,6-dichloro-1H-pyrrolo[2,3-b]pyridine-4-carboxylate

A solution of 5-chloro-4-(methoxycarbonyl)-1H-pyrrolo[2,3-b]pyridine 7-oxide (490 mg, 2.16 mmol, 1.0 eq) in POCl₃ (15.0 mL) was stirred at 70° C. for 8 h. After cooled to room temperature, the reaction mixture concentrated in vacuum. The residue was diluted with DCM (50 mL), washed with aqueous NaHCO₃ solution (50 mL) and brine (50 mL). The organic layer was dried over anhydrous Na₂SO₄, filtered and concentrated under reduced pressure. The crude product was purified by Prep-TLC(PE/EA=4/1 v/v) to obtain methyl 5,6-dichloro-1H-pyrrolo[2,3-b]pyridine-4-carboxylate (60.0 mg, yield 11%) as a white solid. LC-MS (ESI): mass calced for: C₉H₆C₁₂N₂O₂, 243.98; m/z found, 245.1 [M+H]⁺. ¹H NMR (400 MHZ, DMSO-d₆) δ 12.45 (s, 1H), 7.82 (s, 1H), 6.62 (s, 1H), 4.06 (s, 3H).

### Step G: methyl 5,6-dichloro-1-methyl-1H-pyrrolo[2,3-b]pyridine-4-carboxylate

To a solution of methyl 5,6-dichloro-1H-pyrrolo[2,3-b]pyridine-4-carboxylate (60.0 mg, 245 μmol, 1.0 eq) in DMF (2.00 mL) was added NaH (60% suspend in oil) (11.8 mg, 294 μmol, 1.2 eq) at 0° C. and the mixture was stirred for 30 min. MeI (52.1 mg, 367 μmol, 1.5 eq) was added to above mixture and the resulting mixture was stirred at 15° C. for 1 h. The mixture was quenched with saturated aqueous NH₄Cl solution (8 mL) and extracted with EA (5 mL×3). The organic layer was washed with brine (10 mL×4), dried over anhydrous Na₂SO₄, filtered and concentrated under reduced pressure. The crude product was purified by Prep-TLC (PE/EA=4/1 v/v) to obtain methyl 5,6-dichloro-1-methyl-1H-pyrrolo[2,3-b]pyridine-4-carboxylate (40.0 mg, yield 63%) as a white solid. LC-MS (ESI): mass calced for: C₁₀H₈C₁₂N₂O₂, 258.00; m/z found, 259.1 [M+H]⁺

### Step H: (5,6-dichloro-1-methyl-1H-pyrrolo[2,3-b]pyridin-4-yl) methanol

To a solution of methyl 5,6-dichloro-1-methyl-1H-pyrrolo[2,3-b]pyridine-4-carboxylate (40.0 mg, 154 μmol, 1.0 eq) in THF (4.00 mL) was added LiAlH₄ (17.6 mg, 463 μmol, 3.0 eq) at 0° C. and the mixture was stirred at 0° C. for 10 min. The reaction mixture was quenched with Na₂SO₄·10H₂O (150 mg) at 0° C. and stirred for 10 min. After filtration, the filtrate was concentrated in vacuum to afford a crude product (5,6-dichloro-1-methyl-1H-pyrrolo[2,3-b]pyridin-4-yl) methanol (30.0 mg, yield 84%) as a yellow solid. LC-MS (ESI): mass calced for: C₉H&C₁₂N₂O, 230.00; m/z found, 231.1 [M+H]⁺.

### Step I: 5,6-dichloro-1-methyl-1H-pyrrolo[2,3-b]pyridine-4-carbaldehyde

To a solution of (5,6-dichloro-1-methyl-1H-pyrrolo[2,3-b]pyridin-4-yl) methanol (30.0 mg, 130 μmol, 1.0 eq) in DMSO (2.00 mL) was added IBX (Purity 45% W.t) (162 mg, 260 μmol, 2.0 eq). The mixture was stirred at 30° C. for 1 h. The mixture was quenched with saturated aqueous Na₂SO₃ solution (8 mL) and extracted with EA (5 mL×3). The organic layer was washed with aqueous NaHCO₃ solution (8 mL), dried over anhydrous Na₂SO₄, filtered and concentrated under reduced pressure to afford a crude product 5,6-dichloro-1-methyl-1H-pyrrolo[2,3-b]pyridine-4-carbaldehyde (30.0 mg, yield 99%) as a yellow solid. LC-MS (ESI): mass calced for: C₉H₆C₁₂N₂O, 227.99; m/z found, 229.1 [M+H]⁺.

### Intermediate 108:4-(bromomethyl)-2-chloro-8-(2-methoxyethoxy)-1,5-naphthyridine

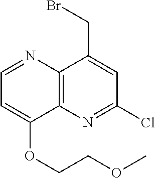 [SMILES: COCCOc1ccnc2c(CBr)cc(Cl)nc12]

### Step A: 6-chloro-8-methyl-1,5-naphthyridin-4-ol

To a solution of 6-chloro-8-methyl-1,5-naphthyridin-4-ol (200 mg, 1.03 mmol, 1.0 eq) and Cs₂CO₃ (1.00 g, 3.08 mmol, 3.0 eq) in DMF (3.00 mL) was added 1-bromo-2-methoxyethane (428 mg, 3.08 mmol, 3.0 eq). The reaction mixture was stirred at 80° C. for 3 h. The reaction mixture was cooled to room temperature and diluted with ethyl acetate (50 mL). The organic layer was washed with brine (50 mL×4), dried over anhydrous sodium sulfate, filtered and concentrated under reduced pressure to obtain 2-chloro-8-(2-methoxyethoxy)-4-methyl-1,5-naphthyridine (140 mg, yield 54%) as a brown solid. LC-MS (ESI): mass calced for: C₁₂H₁₃ClN₂O₂, 252.07; m/z found, 253.0 [M+H]⁺. ¹H NMR (400 MHZ, DMSO-d₆) δ 8.81 (s, 1H), 7.73 (s, 1H), 7.33 (s, 1H), 4.39 (s, 2H), 3.82 (s, 2H), 3.36 (s, 3H), 2.71 (s, 3H).

### Step B: 4-(bromomethyl)-2-chloro-8-(2-methoxyethoxy)-1,5-naphthyridine

To a solution of 2-chloro-8-(2-methoxyethoxy)-4-methyl-1,5-naphthyridine (50.0 mg, 198 μmol, 1.0 eq) and 1-bromopyrrolidine-2,5-dione (77.4 mg, 436 μmol, 2.1 eq) in CCl₄ (1.50 mL) was added AIBN (6.50 mg, 79.2 μmol, 0.4 eq). The reaction mixture was stirred under N₂ atmosphere at 90° C. for 2 h. After evaporation, the residue was diluted with EA (5 mL), washed with saturated aqueous Na₂SO₃ solution (5 mL×2) and brine (5 mL), dried over anhydrous sodium sulfate, filtered and concentrated under reduced pressure. The residue was purified by Prep-TLC(EA/PE=2/3 v/v) to obtain 4-(bromomethyl)-2-chloro-8-(2-methoxyethoxy)-1,5-naphthyridine (15.0 mg, yield 23%) as a white solid. LC-MS (ESI): mass calced for: C₁₂H₁₂BrClN₂O₂, 329.97; m/z found, 331.1 [M+H]⁺.

### Intermediate 109:4-(bromomethyl)-2-chloro-8-((1,1,1-trifluoropropan-2-yl)oxy)-1,5-naphthyridine

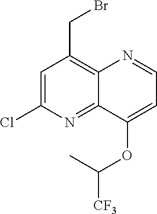 [SMILES: CC(Oc1ccnc2c(CBr)cc(Cl)nc12)C(F)(F)F]

### Step A: 1,1,1-trifluoropropan-2-yl trifluoromethanesulfonate

To a solution of 1,1,1-trifluoropropan-2-ol (400 mg, 3.51 mmol, 1.0 eq) in DCM (6.00 mL) were added pyridine (305 mg, 312 μL, 3.86 mmol, 1.1 eq). Tf₂O (989 mg, 592 μL, 3.51 mmol, 1.0 eq) was add dropwise to above mixture at 0° C. and the reaction mixture was stirred at 15° C. for 2 h. After filtered, the solution was directly used in next step without further purification. LC-MS (ESI): mass calced for: C₄H₄F₆O₃S, 246.12; m/z found, no mass [M+H]⁺.

### Step B: 2-chloro-4-methyl-8-((1,1,1-trifluoropropan-2-yl)oxy)-1,5-naphthyridine

To a solution of 6-chloro-8-methyl-1,5-naphthyridin-4-ol (300 mg, 1.54 mmol, 1.0 eq) in DMF (6.00 mL) was added Cs₂CO₃ (1.00 g, 3.08 mmol, 2.0 eq). The mixture was stirred at 20° C. for 30 min. 1,1,1-trifluoropropan-2-yl trifluoromethanesulfonate was added to above mixture at 0° C. and the mixture was stirred at 80° C. for 3 h. After cooled to room temperature, the reaction mixture was quenched with saturated aqueous NH₄Cl solution (15 mL) and extracted with EA (10 mL×3). The organic layer was washed with brine (50 mL×4), dried over MgSO₄, filtered and concentrated under reduced pressure. The crude product was purified by flash column chromatography on silica gel (ethyl acetate in petroleum ether, 25% v/v) to obtain product 2-chloro-4-methyl-8-((1,1,1-trifluoropropan-2-yl)oxy)-1,5-naphthyridine (180 mg, yield 40%) as a white solid. LC-MS (ESI): mass calced for: C₁₂H₁₀ClF₃N₂O, 290.67; m/z found, 291.3 [M+H]⁺. ¹H NMR (400 MHZ, DMSO-d₆) δ 8.88 (d, J=5.2 Hz, 1H), 7.77 (d, J=0.8 Hz, 1H), 7.58 (d, J=5.2 Hz, 1H), 5.76-5.66 (m, 1H), 2.72 (s, 3H), 1.57 (d, J=6.4 Hz, 3H).

### Step C: 4-(bromomethyl)-2-chloro-8-((1,1,1-trifluoropropan-2-yl)oxy)-1,5-naphthyridine

To a solution of 2-chloro-4-methyl-8-((1,1,1-trifluoropropan-2-yl)oxy)-1,5-naphthyridine (180.0 mg, 619.3 μmol, 1.0 eq) in CCl₄ (7.00 mL) were added NBS (132.3 mg, 743.1 μmol, 1.2 eq) and Benzoyl peroxide (15.00 mg, 61.93 μmol, 0.1 eq). The mixture was stirred at 90° C. for 8 h. After cooled to room temperature, the reaction mixture was quenched with water (10 mL) and extracted with DCM (10 mL×3). The organic layer was dried over MgSO₄, filtered and concentrated under reduced pressure. The crude product was purified by Prep-TLC(PE/EA=2/1 v/v) to obtain 4-(bromomethyl)-2-chloro-8-((1,1,1-trifluoropropan-2-yl)oxy)-1,5-naphthyridine (65.0 mg, yield 28%) as a white solid. LC-MS (ESI): mass calced for: C₁₂H₉BrClF3N₂O, 369.57; m/z found, 370.2 [M+H]⁺.

### Intermediate 110:4-(bromomethyl)-2-chloro-8-((1r,3r)-3-methoxycyclobutoxy)-1,5-naphthyridine

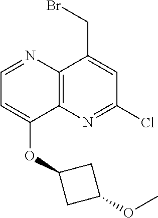 [SMILES: CO[C@H]1C[C@H](Oc2ccnc3c(CBr)cc(Cl)nc23)C1]

### Step A: 2-chloro-8-((1r,3r)-3-methoxycyclobutoxy)-4-methyl-1,5-naphthyridine (Xa) and 2-chloro-8-((1s,3s)-3-methoxycyclobutoxy)-4-methyl-1,5-naphthyridine (Xb)

To a solution of 6-chloro-8-methyl-1,5-naphthyridin-4-ol (100.0 mg, 513.8 μmol, 1.0 eq) in DMF (3.00 mL) were added K₂CO₃ (213.0 mg, 1.541 mmol, 3.0 eq) and 3-methoxycyclobutyl methanesulfonate (277.8 mg, 1.541 mmol, 3.0 eq). The mixture was stirred at 80° C. for 16 h. The reaction mixture was cooled to room temperature, diluted with saturated aqueous NH₄Cl solution (10 mL), and extracted with EA (15 mL×3). The organic layer was washed with brine (15 mL×4), dried over anhydrous Na₂SO₄, filtered and concentrated in vacuum. The residue was purified by Prep-TLC(PE/EtOAc=1/1 v/v) to obtain 2-chloro-8-((1r,3r)-3-methoxycyclobutoxy)-4-methyl-1,5-naphthyridine (Xa) (48.0 mg, yield 33%) as a brown solid and 2-chloro-8-((1s,3s)-3-methoxycyclobutoxy)-4-methyl-1,5-naphthyridine (Xb) (21.0 mg, yield 15%) as a brown solid

2-chloro-8-((1r,3r)-3-methoxycyclobutoxy)-4-methyl-1,5-naphthyridine (Xa): LC-MS (ESI): mass calced for: C₁₄H₁₅ClN₂O₂, 278.08; m/z found, 279.1 [M+H]⁺. ¹H NMR (400 MHZ, DMSO-d₆) δ 8.78 (d, J=3.2 Hz, 1H), 7.73 (s, 1H), 7.09 (d, J=3.2 Hz, 1H), 5.13-5.10 (m, 1H), 4.19-4.07 (m, 1H), 3.20 (s, 3H), 2.71 (s, 3H), 2.61-2.53 (m, 2H), 2.47-2.43 (m, 2H).

2-chloro-8-((1s,3s)-3-methoxycyclobutoxy)-4-methyl-1,5-naphthyridine (Xb): LC-MS (ESI): mass calced for: C₁₄H₁₅ClN₂O₂, 278.08; m/z found, 279.1 [M+H]⁺. ¹H NMR (400 MHz, DMSO-d₆) δ 8.79 (d, J=3.2 Hz, 1H), 7.74 (s, 1H), 7.15 (d, J=3.2 Hz, 1H), 4.71-4.64 (m, 1H), 3.74-3.67 (m, 1H), 3.19 (s, 3H), 3.02-3.01 (m, 2H), 2.71 (s, 3H), 2.08-2.04 (m, 2H).

### Step B: 4-(bromomethyl)-2-chloro-8-((lr,3r)-3-methoxycyclobutoxy)-1,5-naphthyridine

To a stirred mixture of 2-chloro-8-((1r,3r)-3-methoxycyclobutoxy)-4-methyl-1,5-naphthyridine (57.0 mg, 204 μmol, 1.0 eq) in CCl₄ (3.00 mL) were added AIBN (6.72 mg, 40.9 μmol, 0.2 eq) and NBS (54.6 mg, 307 μmol, 1.5 eq). The resulting mixture was stirred under N₂ at 80° C. for 3 h. The reaction mixture was cooled to room temperature, quenched with saturated aqueous Na₂S₂O₃ solution (20 mL), and extracted with DCM (20 mL×3). The combined organic layers were washed with brine (80 mL), dried over anhydrous Na₂SO₄, filtered and concentrated in vacuum. The residue was purified by Prep-TLC(PE/EA=1/1 v/v) to give 4-(bromomethyl)-2-chloro-8-((1r,3r)-3-methoxycyclobutoxy)-1,5-naphthyridine (25.0 mg, yield 34%) as a yellow oil. LC-MS (ESI): mass calced for: C₁₄H₁₄BrClN₂O₂, 355.99; m/z found, 357.1 [M+H]⁺.

### Intermediate 111:4-(bromomethyl)-2-chloro-8-((1s,3s)-3-methoxycyclobutoxy)-1,5-naphthyridine

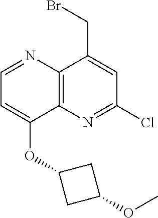 [SMILES: CO[C@H]1C[C@@H](Oc2ccnc3c(CBr)cc(Cl)nc23)C1]

To a stirred mixture of 2-chloro-8-((1s,3s)-3-methoxycyclobutoxy)-4-methyl-1,5-naphthyridine (43.0 mg, 154 μmol, 1.0 eq) in CCl₄ (8.00 mL) were added AIBN (5.07 mg, 30.9 μmol, 0.2 eq) and NBS (41.2 mg, 231 μmol, 1.5 eq). The resulting mixture was stirred under N₂ at 80° C. for 3 h. The reaction mixture was cooled to room temperature, quenched with saturated aqueous Na₂S₂O₃ solution (20 mL), and extracted with DCM (20 mL×3). The combined organic layers were washed with brine (80 mL), dried over anhydrous Na₂SO₄, filtered and concentrated in vacuum. The residue was purified by Prep-TLC(PE/EA=1/1 v/v) to give 4-(bromomethyl)-2-chloro-8-((1s,3s)-3-methoxycyclobutoxy)-1,5-naphthyridine (20.0 mg, yield 36%) as a yellow oil. LC-MS (ESI): mass calced for: C₁₄H₁₄BrClN₂O₂, 355.99; m/z found, 357.1 [M+H]⁺.

### Intermediate 112:4-(bromomethyl)-8-(2-((tert-butyldimethylsilyl)oxy) ethoxy)-2-chloro-1,5-naphthyridine

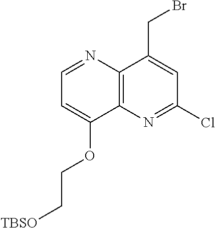 [SMILES: CC(C)(C)[Si](C)(C)OCCOc1ccnc2c(CBr)cc(Cl)nc12]

### Step A: 8-(2-((tert-butyldimethylsilyl)oxy) ethoxy)-2-chloro-4-methyl-1,5-naphthyridine

To a solution of 6-chloro-8-methyl-1,5-naphthyridin-4-ol (1.00 g, 5.14 mmol, 1.0 eq) in DMF (20.0 mL) was added cesium carbonate (5.02 g, 15.4 mmol, 3.0 eq) and the mixture was stirred at 80° C. for 1 h. Then (2-bromoethoxy) (tert-butyl)dimethylsilane (1.84 g, 7.71 mmol, 1.5 eq) was added to above mixture and the mixture was stirred at 80° C. overnight. After cooled to room temperature, the mixture was poured into water (80 mL) and extracted with EA (40 mL×3). The organic layer was washed with brine (40 mL×4), dried over MgSO₄, filtered and concentrated under reduced pressure. The residue was purified by flash column chromatography on silica gel (PE/EA=3/1 v/v) to give 8-(2-((tert-butyldimethylsilyl)oxy) ethoxy)-2-chloro-4-methyl-1,5-naphthyridine (1.40 g, yield 70%) as a yellow oil. LC-MS (ESI): mass calced for: C₁₇H₂₅ClN₂O₂Si, 352.14; m/z found, 353.2 [M+H]⁺.

### Step B: 8-(2-((tert-butyldimethylsilyl)oxy) ethoxy)-2-chloro-4-methyl-1,5-naphthyridine

To a solution of 8-(2-((tert-butyldimethylsilyl)oxy) ethoxy)-2-chloro-4-methyl-1,5-naphthyridine (1.40 g, 3.97 mmol, 1.0 eq) and AIBN (195 mg, 1.19 mmol, 0.3 eq) in CCl₄ (20.0 mL) was added NBS (1.41 g, 7.93 mmol, 2.0 eq). The mixture was stirred under N₂ at 95° C. for 16 h. The reaction mixture was cooled to room temperature and concentrated under reduced pressure. The residue was poured into water (15 mL) and extracted with EtOAc (20 mL×3). The organic layer was dried over MgSO₄, filtered and concentrated under reduced pressure. The residue was purified by flash column chromatography on silica gel (ethyl acetate in petroleum ether, 30% v/v) to give obtain 4-(bromomethyl)-8-(2-((tert-butyldimethylsilyl)oxy) ethoxy)-2-chloro-1,5-naphthyridine (200 mg, yield 11%) as a yellow solid. LC-MS (ESI): mass calced for: C₁₇H₂₄BrClN₂O₂Si, 430.05; m/z found, 431.2 [M+H]⁺.

### Intermediate 113:5-chloro-3-(ethylamino)-2-((2-(trimethylsilyl)ethoxy)methyl)-2H-pyrazolo[4,3-b]pyridine-7-carbaldehyde

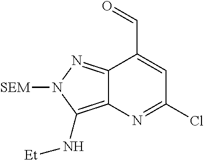 [SMILES: CCNc1c2nc(Cl)cc(C=O)c2nn1COCC[Si](C)(C)C]

### Step A: methyl 5-chloro-3-(ethylamino)-2-((2-(trimethylsilyl)ethoxy)methyl)-2H-pyrazolo[4,3-b]pyridine-7-carboxylate

To a solution of methyl 3-amino-5-chloro-2-((2-(trimethylsilyl)ethoxy)methyl)-2H-pyrazolo[4,3-b]pyridine-7-carboxylate (2.00 g, 5.60 mmol, 1.0 eq) in MeOH (20.0 mL) were added Acetaldehyde (30% W.t) (6.72 mL, 33.6 mmol, 6.0 eq) and Acetic acid (673 mg, 644 μL, 11.2 mmol, 2.0 eq) at room temperature and the mixture was stirred at 50° C. for 4 h. Then Sodium cyanoborohydride (1.76 g, 28.0 mmol, 5.0 eq) was added in portions to above mixture and the resulting reaction mixture was stirred at 50° C. for 16 h. After evaporation, the residue was diluted with EA (200 mL), washed with water (200 mL) and brine (200 mL), dried over anhydrous Na₂SO₄, filtered and concentrated under reduced pressure. The crude product was purified by flash column chromatography on silica gel (PE/EA=3/1 v/v) to give methyl 5-chloro-3-(ethylamino)-2-((2-(trimethylsilyl)ethoxy)methyl)-2H-pyrazolo[4,3-b]pyridine-7-carboxylate (1.10 g, yield 51%) as a yellow solid. LC-MS (ESI): mass calced for: C₁₆H₂₅ClN₄O₃Si, 384.14; m/z found, 385.1 [M+H]⁺.

### Step B: (5-chloro-3-(ethylamino)-2-((2-(trimethylsilyl)ethoxy)methyl)-2H-pyrazolo[4,3-b]pyridin-7-yl) methanol

To a solution of methyl 5-chloro-3-(ethylamino)-2-((2-(trimethylsilyl)ethoxy)methyl)-2H-pyrazolo[4,3-b]pyridine-7-carboxylate (1.10 g, 2.86 mmol, 1.0 eq) in THF (20.0 mL) were added LiAlH₄ (130 mg, 3.43 mmol, 1.2 eq) and the mixture was stirred under N₂ at 0° C. for 10 min. The mixture was quenched with saturated aqueous NH₄Cl solution (5 mL), diluted with water (5 mL), and extracted with EtOAc (10 mL×3). The organic layer was dried over MgSO₄, filtered and concentrated under reduced pressure to obtain crude product (5-chloro-3-(ethylamino)-2-((2-(trimethylsilyl)ethoxy)methyl)-2H-pyrazolo[4,3-b]pyridin-7-yl) methanol (800 mg, yield 71%) as a red solid. The crude product was directly used in next step without further purification. LC-MS (ESI): mass calced for: C₁₅H₂₅ClN₄O₂Si, 356.14; m/z found, 357.1 [M+H]⁺.

### Step C: 5-chloro-3-(ethylamino)-2-((2-(trimethylsilyl)ethoxy)methyl)-2H-pyrazolo[4,3-b]pyridine-7-carbaldehyde

To a solution of (5-chloro-3-(ethylamino)-2-((2-(trimethylsilyl)ethoxy)methyl)-2H-pyrazolo[4,3-b]pyridin-7-yl) methanol (800 mg, 2.24 mmol, 1.0 eq) in DMSO (20.00 mL) was added IBX (816 mg, 2.91 mmol, 1.3 eq) at 25° C. and the mixture was stirred at 25° C. for 1 h. The mixture was quenched with saturated aqueous Na₂SO₃ solution (15 mL), poured into water (15 mL), and extracted with EtOAc (30 mL×3). The organic layer was washed with brine (20 mL×4), dried over MgSO₄, filtered and concentrated under reduced pressure. The residue was purified with flash column chromatography on silica gel (ethyl acetate in petroleum ether, from 0% to 100% v/v) to afford 5-chloro-3-(ethylamino)-2-((2-(trimethylsilyl)ethoxy)methyl)-2H-pyrazolo[4,3-b]pyridine-7-carbaldehyde (600 mg, yield 90%) as a red solid. LC-MS (ESI): mass calced for: C₁₅H₂₃ClN₄O₂Si, 354.13; m/z found, 355.1 [M+H]⁺. ¹H NMR (400 MHZ, DMSO-d₆) δ 10.27 (s, 1H), 7.61 (s, 1H), 7.02 (t, J=5.6 Hz, 1H), 3.83-3.76 (m, 2H), 3.59 (t, J=8.0 Hz, 2H), 1.22 (t, J=7.0 Hz, 3H), 0.84 (t, J=8.0 Hz, 2H),-0.07 (s, 9H).

### Intermediate 114:3-methyl-3-(1-oxo-5-(4,4,5,5-tetramethyl-1,3,2-dioxaborolan-2-yl) isoindolin-2-yl) piperidine-2,6-dione

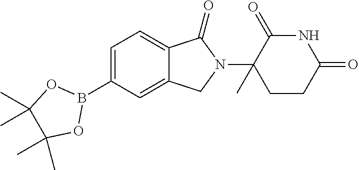 [SMILES: CC1(N2Cc3cc(B4OC(C)(C)C(C)(C)O4)ccc3C2=O)CCC(=O)NC1=O]

### Step A: 3-(5-bromo-1-oxoisoindolin-2-yl)-3-methylpiperidine-2,6-dione

To a solution of 3-amino-3-methylpiperidine-2,6-dione hydrochloride (400 mg, 2.24 mmol, 1.0 eq) and methyl 4-bromo-2-(bromomethyl)benzoate (690 mg, 2.24 mmol, 1.0 eq) in MeCN (10.0 mL) were added DIPEA (868 mg, 1.17 mL, 6.72 mmol, 3.0 eq). The mixture was stirred under N₂ at 85° C. for 16 h. The reaction mixture was cooled to room temperature and concentrated under reduced pressure. The residue was poured into water (5 mL) and extracted with EtOAc (10 mL×3). The organic layer was dried over MgSO₄, filtered and concentrated under reduced pressure. The residue was purified by Prep-TLC(DCM/MeOH=10/1 v/v) to obtain 3-(5-bromo-1-oxoisoindolin-2-yl)-3-methylpiperidine-2,6-dione (300 mg, yield 36%) as a brown solid. LC-MS (ESI): mass calced for: C₁₄H₁₃BrN₂O₃, 336.01; m/z found, 337.2 [M+H]⁺.

### Step B: 3-methyl-3-(1-oxo-5-(4,4,5,5-tetramethyl-1,3,2-dioxaborolan-2-yl) isoindolin-2-yl) piperidine-2,6-dione

To a solution of 3-(5-bromo-1-oxoisoindolin-2-yl)-3-methylpiperidine-2,6-dione (300 mg, 890 μmol, 1.0 eq) and anhydrous Potassium acetate (262 mg, 2.67 mmol, 3.0 eq) in dry 1,4-Dioxane (10.0 mL) were added Bis(pinacolato) diborane (271 mg, 1.07 mmol, 1.2 eq) and PdCl₂ (dppf) (65.1 mg, 89.0 μmol, 0.1 eq). The mixture was stirred under N₂ at 95° C. for 16 h. The reaction mixture was cooled to room temperature and concentrated under reduced pressure. The residue was poured into water (10 mL) and extracted with EtOAc (20 mL×3). The organic layer was dried over MgSO₄, filtered and concentrated under reduced pressure. The residue was purified by Prep-TLC(DCM/MeOH=10/1 v/v) to obtain 3-methyl-3-(1-oxo-5-(4,4,5,5-tetramethyl-1,3,2-dioxaborolan-2-yl) isoindolin-2-yl) piperidine-2,6-dione (230 mg, yield 61%) as a brown solid. LC-MS (ESI): mass calced for: C₂₀H₂₅BN₂O₅, 384.19; m/z found, 385.2 [M+H]⁺

### Intermediate 115:5-chloro-3-(cyclobutylamino)-1-((2-(trimethylsilyl)ethoxy)methyl)-1H-pyrazolo[4,3-b]pyridine-7-carbaldehyde

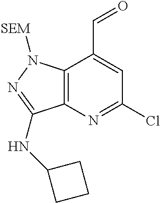 [SMILES: C[Si](C)(C)CCOCn1nc(NC2CCC2)c2nc(Cl)cc(C=O)c21]

### Step A: 7-bromo-5-chloro-N-cyclobutyl-1-((2-(trimethylsilyl)ethoxy)methyl)-1H-pyrazolo[4,3-b]pyridin-3-amine

To a solution of 7-bromo-5-chloro-1-((2-(trimethylsilyl)ethoxy)methyl)-1H-pyrazolo[4,3-b]pyridin-3-amine (4.00 g, 10.58 mmol, 1.0 eq) in MeOH (40.0 mL) was added cyclobutanone (4.46 g, 63.6 mmol, 6.0 eq) and Acetic acid (1.27 g, 21.2 mmol, 2.0 eq) at room temperature and the mixture was stirred at 50° C. for 4 h. Then Sodium cyanoborohydride (3.32 g, 53.0 mmol, 5.0 eq) was added to above mixture in portions and the reaction mixture was stirred at 50° C. for 16 h. After evaporation, the residue was diluted with EA (300 mL), washed with water (300 mL) and brine (300 mL), dried over anhydrous Na₂SO₄, filtered and concentrated under reduced pressure. The crude product was purified by flash column chromatography on silica gel (PE/EA=3/1 v/v) to give 7-bromo-5-chloro-N-cyclobutyl-1-((2-(trimethylsilyl)ethoxy)methyl)-1H-pyrazolo[4,3-b]pyridin-3-amine (2.80 g, yield 61%) as a yellow solid. LC-MS (ESI): mass calced for: C₁₆H₂₄BrClN₄OSi, 430.06; m/z found, 431.1 [M+H]⁺. ¹H NMR (400 MHZ, DMSO-d₆) δ 7.98 (s, 1H), 6.86 (d, J=8.0 Hz, 1H), 5.81 (s, 2H), 4.34-4.28 (m, 1H), 3.71-3.61 (m, 2H), 2.40-2.37 (m, 2H), 2.20-2.14 (m, 2H), 1.86-1.70 (m, 2H), 0.89 (t, J=8.0 Hz, 2H), 0.00 (s, 9H).

### Step B: 5-chloro-N-cyclobutyl-1-((2-(trimethylsilyl)ethoxy)methyl)-7-vinyl-1H-pyrazolo[4,3-b]pyridin-3-amine

To a solution of 7-bromo-5-chloro-N-cyclobutyl-1-((2-(trimethylsilyl)ethoxy)methyl)-1H-pyrazolo[4,3-b]pyridin-3-amine (2.80 g, 6.48 mmol, 1.0 eq) in 1,4-Dioxane (50.0 mL) and water (5.0 mL) were added Potassium phosphate tribasic (4.12 g, 19.46 mmol, 3.0 eq), Potassium vinyl trifluoroborate (1.30 g, 9.72 mmol, 1.5 eq,), and 1,1′— Bis(diphenylphosphino) ferrocene-palladium (II) dichloride (474 mg, 648 μmol, 0.1 eq) at 25° C. The mixture was stirred under N₂ at 80° C. for 3 h. After cooled to room temperature, the reaction mixture was filtrated and the filtrate was diluted with EtOAc (200 mL). The organic layer was washed with brine (200 mL), dried over Na₂SO₄, filtered and concentrated under reduced pressure. The residue was purified by flash column chromatography on silica gel (PE/EtOAc=2/1 v/v) to obtain 5-chloro-N-cyclobutyl-1-((2-(trimethylsilyl)ethoxy)methyl)-7-vinyl-1H-pyrazolo[4,3-b]pyridin-3-amine (1.60 g, yield 65%) as a yellow oil. LC-MS (ESI): mass calced for: C₁₈H₂₇ClN₄OSi, 378.16; m/z found, 379.1 [M+H]⁺.

### Step C: 1-(5-chloro-3-(cyclobutylamino)-1-((2-(trimethylsilyl)ethoxy)methyl)-1H-pyrazolo[4,3-b]pyridin-7-yl) ethane-1,2-diol

To a solution of 5-chloro-N-cyclobutyl-1-((2-(trimethylsilyl)ethoxy)methyl)-7-vinyl-1H-pyrazolo[4,3-b]pyridin-3-amine (1.60 g, 4.22 mmol, 1.0 eq) and N-methylmorpholine N-oxide (989 mg, 8.44 mmol, 2.0 eq) in Acetone (20.0 mL) and water (10.0 mL) was added potassium osmate (VI) dihydrate (156 mg, 422 μmol, 0.1 eq). The reaction was stirred at 25° C. for 16 h. After filtered, the filtrate was concentrated in vacuum and the residue was diluted with EA (100 mL). The organic layer was washed with brine (50 mL), dried over anhydrous Na₂SO₄, filtered and concentrated in vacuum. The residue was purified by flash column chromatography on silica gel (PE/EtOAc=1/1 v/v) to give 1-(5-chloro-3-(cyclobutylamino)-1-((2-(trimethylsilyl)ethoxy)methyl)-1H-pyrazolo[4,3-b]pyridin-7-yl) ethane-1,2-diol (1.40 g, yield 80%) as a black solid. LC-MS (ESI): mass calced for: C₁₈H₂₉ClN₄O₃Si, 412.17; m/z found, 413.2 [M+H].

### Step D: 5-chloro-3-(cyclobutylamino)-1-((2-(trimethylsilyl)ethoxy)methyl)-1H-pyrazolo[4,3-b]pyridine-7-carbaldehyde

To a solution of 1-(5-chloro-3-(cyclobutylamino)-1-((2-(trimethylsilyl)ethoxy)methyl)-1H-pyrazolo[4,3-b]pyridin-7-yl) ethane-1,2-diol (1.40 g, 3.39 mmol, 1.0 eq) in THF (30.0 mL) and Water (10.0 mL) was added Sodium metaperiodate (1.45 g, 6.78 mmol, 2.0 eq). The resulting mixture was stirred at 25° C. for 1 h. The reaction mixture was quenched with saturated aqueous Na₂S₂O₃ solution (100 mL) and extracted with EtOAc (100 mL×3). The combined organic layers were washed with brine (100 mL×3), dried over anhydrous Na₂SO₄, filtered and concentrated. The residue was purified by flash column chromatography on silica gel (PE/EtOAc=1/1 v/v) to give 5-chloro-3-(cyclobutylamino)-1-((2-(trimethylsilyl)ethoxy)methyl)-1H-pyrazolo[4,3-b]pyridine-7-carbaldehyde (1.10 g, yield 85%) as a red solid. LC-MS (ESI): mass calced for: C₁₇H₂₅ClN₄O₂Si, 380.14; m/z found, 381.1 [M+H]. ¹H NMR (400 MHZ, DMSO-d₆) δ 10.41 (s, 1H), 8.08 (s, 1H), 6.99 (d, J=8.0 Hz, 1H), 5.92 (s, 2H), 4.41-4.29 (m, 1H), 3.60-3.57 (m, 2H), 2.44-2.39 (m, 2H), 2.24-2.20 (m, 2H), 1.86-1.78 (m, 2H), 0.90-0.84 (m, 2H),-0.01 (s, 9H).

### Intermediate 116:5-chloro-3-(methylamino)-2-((2-(trimethylsilyl)ethoxy)methyl)-2H-pyrazolo[4,3-b]pyridine-7-carbaldehyde

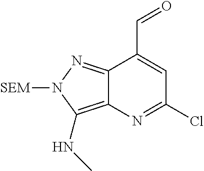 [SMILES: CNc1c2nc(Cl)cc(C=O)c2nn1COCC[Si](C)(C)C]

### Step A: methyl 5-chloro-3-(methylamino)-2-((2-(trimethylsilyl)ethoxy)methyl)-2H-pyrazolo[4,3-b]pyridine-7-carboxylate

To a solution of methyl 3-amino-5-chloro-2-((2-(trimethylsilyl)ethoxy)methyl)-2H-pyrazolo[4,3-b]pyridine-7-carboxylate (1.00 g, 2.80 mmol, 1.0 eq) in MeOH (10.0 mL) were added aqueous formaldehyde solution (37% W.t) (505 mg, 16.8 mmol, 6.0 eq) and Acetic acid (337 mg, 5.60 mmol, 2.0 eq). The reaction mixture was stirred at 50° C. for 4 h. Sodium cyanoborohydride (880 mg, 14.0 mmol, 5.0 eq) was added to above mixture and the resulting reaction mixture was stirred at 50° C. for 20 h. The mixture was concentrated under reduced pressure and the residue was diluted with EA (40 mL). The organic layer was washed with water (20 mL) and brine (20 mL), dried over anhydrous Na₂SO₄, filtered and concentrated under reduced pressure. The crude product was purified by flash column chromatography on silica gel (ethyl acetate in petroleum ether, 15% v/v) to give methyl 5-chloro-3-(methylamino)-2-((2-(trimethylsilyl)ethoxy)methyl)-2H-pyrazolo[4,3-b]pyridine-7-carboxylate (500 mg, yield 48%) as a yellow solid. LC-MS (ESI): mass calced for: C₁₅H₂₃ClN₄O₃Si, 370.12; m/z found, 371.0 [M+H]⁺

### Step B: (5-chloro-3-(methylamino)-2-((2-(trimethylsilyl)ethoxy)methyl)-2H-pyrazolo[4,3-b]pyridin-7-yl) methanol

To a solution of methyl 5-chloro-3-(methylamino)-2-((2-(trimethylsilyl)ethoxy)methyl)-2H-pyrazolo[4,3-b]pyridine-7-carboxylate (500.0 mg, 1.348 mmol, 1.0 eq) in THF (10.00 mL) was added LiAlH₄ (76.74 mg, 2.022 mmol, 1.5 eq) at 0° C. The mixture was stirred at 0° C. for 10 min. The reaction mixture was quenched with Na₂SO_{4 10}H₂O (150 mg) at 0° C. and stirred for 10 min. After filtration, the filtrate was concentrated in vacuum to afford a crude product (5-chloro-3-(methylamino)-2-((2-(trimethylsilyl)ethoxy)methyl)-2H-pyrazolo[4,3-b]pyridin-7-yl) methanol (450 mg, yield 97%) as a yellow oil. The crude product was directly used in next step without further purification. LC-MS (ESI): mass calced for: C₁₄H₂₃ClN₄O₂Si, 342.13; m/z found, 343.1 [M+H].

### Step C: 5-chloro-3-(methylamino)-2-((2-(trimethylsilyl)ethoxy)methyl)-2H-pyrazolo[4,3-b]pyridine-7-carbaldehyde

To a solution of (5-chloro-3-(methylamino)-2-((2-(trimethylsilyl)ethoxy)methyl)-2H-pyrazolo[4,3-b]pyridin-7-yl) methanol (450.0 mg, 1.312 mmol, 1.0 eq) in DMSO (10.00 mL) was added IBX (Purity: 45% W.t) (1.633 g, 2.625 mmol, 2.0 eq). The reaction mixture was stirred at 30° C. for 1 h. After quenched with saturated aqueous Na₂SO₃ solution (40 mL) and extracted with EA (20 mL×3). The organic layer was washed with saturated aqueous NaHCO₃ solution (40 mL), dried over anhydrous Na₂SO₄, filtered and concentrated under reduced pressure. The crude product was purified by flash column chromatography on silica gel (ethyl acetate in petroleum ether, 15% v/v) to give 5-chloro-3-(methylamino)-2-((2-(trimethylsilyl)ethoxy)methyl)-2H-pyrazolo[4,3-b]pyridine-7-carbaldehyde (250 mg, yield 55%) as a red solid. LC-MS (ESI): mass calced for: C₁₄H₂₁ClN₄O₂Si, 340.11; m/z found, 341.2 [M+H]⁺

### Intermediate 117: N-(5-chloro-7-(pyrrolidin-1-ylmethyl)-1-((2-(trimethylsilyl)ethoxy)methyl)-1H-pyrazolo[4,3-b]pyridin-3-yl) methanesulfonamide

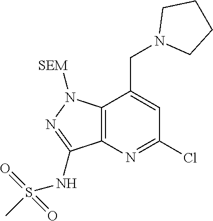 [SMILES: C[Si](C)(C)CCOCn1nc(NS(C)(=O)=O)c2nc(Cl)cc(CN3CCCC3)c21]

### Step A: N-(7-bromo-5-chloro-1-((2-(trimethylsilyl)ethoxy)methyl)-1H-pyrazolo[4,3-b]pyridin-3-yl)-N-(methylsulfonyl) methanesulfonamide

A solution of 7-bromo-5-chloro-1-((2-(trimethylsilyl)ethoxy)methyl)-1H-pyrazolo[4,3-b]pyridin-3-amine (3.00 g, 7.94 mmol, 1.0 eq), DMAP (48.5 mg, 397 μmol, 0.05 eq), and DIPEA (3.08 g, 23.8 mmol, 3 eq) in DCM (30.00 mL) was stirred at 20° C. for 5 min, then methanesulfonyl chloride (2.27 g, 19.9 mmol, 2.5 eq) was added dropwise to above mixture at 0° C. The reaction mixture was stirred at 20° C. for 3 h, diluted with water (30 mL), and extracted with DCM (30 mL×3). The organic phase was washed with diluted aqueous hydrochloric acid solution (1 N) (30 mL×2), saturated aquoeus NaHCO₃ solution (30 mL) and brine (50 mL), dried over anhydrous Na₂SO₄, filtered and concentrated in vacuum. The residue was purified by flash column chromatography on silica gel (ethyl acetate in petroleum ether, 28% v/v) to give N-(7-bromo-5-chloro-1-((2-(trimethylsilyl)ethoxy)methyl)-1H-pyrazolo[4,3-b]pyridin-3-yl)-N-(methylsulfonyl) methane sulfonamide (3.50 g, yield 82%) as a yellow solid. LC-MS (ESI): mass calced for: C₁₄H₂₂BrClN₄O₅S₂Si, 531.97; m/z found, 533.0 [M+H]⁺.

### Step B: N-(5-chloro-1-((2-(trimethylsilyl)ethoxy)methyl)-7-vinyl-1H-pyrazolo[4,3-b]pyridin-3-yl)-N-(methylsulfonyl) methanesulfonamide

To a solution of N-(7-bromo-5-chloro-1-((2-(trimethylsilyl)ethoxy)methyl)-1H-pyrazolo[4,3-b]pyridin-3-yl)-N-(methylsulfonyl) methanesulfonamide (3.50 g, 6.56 mmol, 1.0 eq) in Dioxane (40.0 mL) and water (4.0 mL) were added potassium phosphate (4.17 g, 19.7 mmol, 3.0 eq), potassium vinyl trifluoroborate (1.32 g, 9.83 mmol, 1.5 eq), and 1,1′— bis(diphenylphosphino) ferrocene-palladium (II) dichloride (480 mg, 656 μmol, 0.1 eq) at 25° C. The mixture was stirred under N₂ at 85° C. for 1 h. After cooled to room temperature, the reaction mixture was filtrated and the filtrate was diluted with EtOAc (100 mL). The organic layer was washed with brine (100 mL), dried over anhydrous Na₂SO₄, filtered and concentrated under reduced pressure. The residue was purified by flash column chromatography on silica gel (PE/EtOAc=3/1 v/v) to obtain N-(5-chloro-1-((2-(trimethylsilyl)ethoxy)methyl)-7-vinyl-1H-pyrazolo[4,3-b]pyridin-3-yl)-N-(methylsulfonyl) methanesulfonamide (2.00 g, yield 63%) as a yellow solid. LC-MS (ESI): mass calced for: C₁₆H₂₅ClN₄O₅S₂Si, 480.07; m/z found, 481.1 [M+H]⁺. ¹H NMR (400 MHZ, DMSO-d₆) δ 7.96 (s, 1H), 7.47 (dd, J=17.2, 11.2 Hz, 1H), 6.49 (d, J=17.2 Hz, 1H), 6.02-5.95 (m, 3H), 3.85 (s, 6H), 3.65-3.55 (m, 2H), 0.95-0.81 (m, 2H), −0.02 (s, 9H).

### Step C: N-(5-chloro-7-(1,2-dihydroxyethyl)-1-((2-(trimethylsilyl)ethoxy)methyl)-1H-pyrazolo[4,3-b]pyridin-3-yl)-N-(methylsulfonyl) methane sulfonamide

To a solution of N-(5-chloro-1-((2-(trimethylsilyl)ethoxy)methyl)-7-vinyl-1H-pyrazolo[4,3-b]pyridin-3-yl)-N-(methylsulfonyl) methanesulfonamide (2.00 g, 4.16 mmol, 1.0 eq) and N-methylmorpholine N-oxide (974 mg, 8.32 mmol, 2 eq) in Acetone (20.0 mL) and water (10.0 mL) was added potassium osmate (VI) dihydrate (153 mg, 416 μmol, 0.1 eq). The reaction was stirred at 25° C. for 16 h. After filtration, the filtrate was diluted with H₂O (20 mL) and extracted with EA (40 mL×3). The organic phase was dried over anhydrous Na₂SO₄, filtered and concentrated under reduced pressure. The residue was purified by flash column chromatography on silica gel (PE/EtOAc=1/1 v/v) to give N-(5-chloro-7-(1,2-dihydroxyethyl)-1-((2-(trimethylsilyl)ethoxy)methyl)-1H-pyrazolo[4,3-b]pyridin-3-yl)-N-(methylsulfonyl) methanesulfonamide (1.40 g, yield 65%) as a yellow solid. LC-MS (ESI): mass calced for: C₁₆H₂₇ClN₄O₇S₂Si, 514.08; m/z found, 515.1 [M+H]⁺.

### Step D: N-(5-chloro-7-formyl-1-((2-(trimethylsilyl)ethoxy)methyl)-1H-pyrazolo[4,3-b]pyridin-3-yl)-N-(methylsulfonyl) methanesulfonamide

To a stirred mixture of N-(5-chloro-7-(1,2-dihydroxyethyl)-1-((2-(trimethylsilyl)ethoxy)methyl)-1H-pyrazolo[4,3-b]pyridin-3-yl)-N-(methylsulfonyl) methanesulfonamide (1.80 g, 3.49 mmol, 1.0 eq) in THF (30.0 mL) and Water (10.0 mL) was added Sodium metaperiodate (1.49 g, 6.99 mmol, 2.0 eq). The resulting mixture was stirred at 25° C. for 1 h. The reaction mixture was quenched with saturated aqueous Na₂S₂O₃ solution (50 mL) and extracted with EtOAc (50 mL×3). The combined organic layers were washed with brine (50 mL×3), dried over anhydrous Na₂SO₄, filtered and concentrated under reduced pressure. The residue was purified by flash column chromatography on silica gel (PE/EtOAc=1/1 v/v) to give N-(5-chloro-7-formyl-1-((2-(trimethylsilyl)ethoxy)methyl)-1H-pyrazolo[4,3-b]pyridin-3-yl)-N-(methylsulfonyl) methane sulfonamide (1.20 g, yield 71%) as a yellow solid. LC-MS (ESI): mass calced for: C₁₅H₂₃ClN₄O₆S₂Si, 482.05; m/z found, 483.1 [M+H]. ¹H NMR (400 MHZ, DMSO-d₆) δ 10.51 (s, 1H), 8.28 (s, 1H), 6.22 (s, 2H), 3.88 (s, 6H), 3.59-3.54 (m, 2H), 0.93-0.82 (m, 2H), 0.00 (s, 9H).

### Step E: N-(5-chloro-7-(pyrrolidin-1-ylmethyl)-1-((2-(trimethylsilyl)ethoxy)methyl)-1H-pyrazolo[4,3-b]pyridin-3-yl)-N-(methylsulfonyl) methane sulfonamide

To a solution of N-(5-chloro-7-formyl-1-((2-(trimethylsilyl)ethoxy)methyl)-1H-pyrazolo[4,3-b]pyridin-3-yl)-N-(methylsulfonyl) methanesulfonamide (300 mg, 621 μmol, 1.0 eq) and pyrrolidine (66.3 mg, 932 μmol, 1.5 eq) in DCM (10.0 mL) was added Acetic acid (37.3 mg, 621 μmol, 1.0 eq) at room temperature and the reaction mixture was stirred at 15° C. for 16 h. Then Sodium triacetoxyborohydride (395 mg, 1.86 mmol, 3.0 eq) was added to above mixture and the reaction mixture was stirred at 15° C. for 2 h. The mixture was quenched with water (50 mL) and extracted with DCM (50 mL×3). The organic layers were washed with brine (50 mL×3), dried over anhydrous Na₂SO₄, filtered and concentrated under reduced pressure. The residue was purified by Prep-TLC(PE/EA=2/1 v/v) to give N-(5-chloro-7-(pyrrolidin-1-ylmethyl)-1-((2-(trimethylsilyl)ethoxy)methyl)-1H-pyrazolo[4,3-b]pyridin-3-yl)-N-(methylsulfonyl) methane sulfonamide (187 mg, yield 56%) as a white solid. LC-MS (ESI): mass calced for: C₁₉H₃₂ClN₅O₅S₂Si, 537.13; m/z found, 538.2 [M+H]⁺.

### Step F: N-(5-chloro-7-(pyrrolidin-1-ylmethyl)-1-((2-(trimethylsilyl)ethoxy)methyl)-1H-pyrazolo[4,3-b]pyridin-3-yl) methanesulfonamide

To a solution of N-(5-chloro-7-(pyrrolidin-1-ylmethyl)-1-((2-(trimethylsilyl)ethoxy)methyl)-1H-pyrazolo[4,3-b]pyridin-3-yl)-N-(methylsulfonyl) methanesulfonamide (187.0 mg, 347.5 μmol, 1.0 eq) in DCM (5.00 mL) was added a solution of potassium carbonate (240.1 mg, 1.74 mmol, 5.0 eq) in MeOH (5.0 mL) and the solution was stirred at 20° C. for 20 min. The reaction mixture was filtered and the filtrate was concentrated under reduced pressure. The residue was purified with Prep-TLC(EA/PE=2/1 v/v) to give N-(5-chloro-7-(pyrrolidin-1-ylmethyl)-1-((2-(trimethylsilyl)ethoxy)methyl)-1H-pyrazolo[4,3-b]pyridin-3-yl) methanesulfonamide (110 mg, yield 69%) as a yellow oil. LC-MS (ESI): mass calced for: C₁₈H₃₀ClN₅O₃SSi, 459.15; m/z found, 460.1 [M+H]. ¹H NMR (400 MHz, DMSO-d₆) δ 10.61 (s, 1H), 7.64 (s, 1H), 5.94 (s, 2H), 4.06 (s, 2H), 3.60 (t, J=8.0 Hz, 2H), 3.42 (s, 3H), 2.61 (s, 4H), 1.83 (s, 4H), 0.89 (t, J=8.0 Hz, 2H),-0.00 (s, 9H).

### Intermediate 118:1-(1-oxo-5-(4,4,5,5-tetramethyl-1,3,2-dioxaborolan-2-yl) isoindolin-2-yl)-3-azabicyclo[3.1.1]heptane-2,4-dione

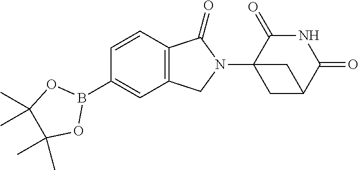 [SMILES: CC1(C)OB(c2ccc3c(c2)CN(C24CC(C2)C(=O)NC4=O)C3=O)OC1(C)C]

### Step A: methyl 3-cyano-3-((4-methoxybenzyl)amino) cyclobutane-1-carboxylate

To a solution of methyl 3-oxocyclobutane-1-carboxylate (10.0 g, 78.0 mmol, 1.0 eq) in MeOH (100.0 mL) was added (4-methoxyphenyl) methanamine (11.8 g, 11.2 mL, 85.9 mmol, 1.1 eq). The mixture was stirred 25° C. for 1 h. Then trimethylsilyl nitrile (15.5 g, 156 mmol, 2.0 eq) was added dropwise to above solution at 0° C. and the resulting reaction mixture was stirred at 25° C. for 16 h. After evaporation, the crude product was purified by flash column chromatography on silica gel (ethyl acetate in petroleum ether, 46% v/v) to obtain methyl 3-cyano-3-((4-methoxybenzyl)amino) cyclobutane-1-carboxylate (15.0 g, yield 70%) as a yellow oil. LC-MS (ESI): mass calced for: C₁₅H₁₈N₂O₃, 274.32; m/z found, 275.3 [M+H]⁺.

### Step B: 1-((4-methoxybenzyl)amino)-3-azabicyclo[3.1.1]heptane-2,4-dione

To the solution of methyl 3-cyano-3-((4-methoxybenzyl)amino) cyclobutane-1-carboxylate (5.00 g, 18.2 mmol, 1.0 eq) in AcOH (55.0 mL) was added con. H₂SO₄ (8.00 mL). The reaction mixture was stirred at 45° C. for 16 h. After cooled to room temperature, the reaction solution was poured into ice water (200.0 mL), cautiously adjusted to pH 7 with sodium bicarbonate, and extract extracted with EA (200 mL×3). The organic layer was washed with aqueous NaHCO₃ solution (100 mL) and brine (100 mL), dried over anhydrous Na₂SO₄, filtered and concentrated under reduced pressure. The crude product was purified by flash column chromatography on silica gel (MeOH in DCM, 4% v/v) to obtain 1-((4-methoxybenzyl)amino)-3-azabicyclo[3.1.1]heptane-2,4-dione (1.20 g, yield 25%) as a white solid. LC-MS (ESI): mass calced for: C₁₄H₁₆N₂O₃, 260.29; m/z found, 261.3 [M+H]⁺. ¹H NMR (400 MHZ, DMSO-d₆) δ 7.25 (d, J=8.6 Hz, 2H), 6.88 (d, J=8.6 Hz, 2H), 3.74 (s, 3H), 3.59 (s, 2H), 3.13-3.04 (m, 1H), 2.60-2.57 (m, 2H), 2.31-2.23 (m, 2H).

### Step C: 1-amino-3-azabicyclo[3.1.1]heptane-2,4-dione hydrochloride

To a stirred solution of 1-((4-methoxybenzyl)amino)-3-azabicyclo[3.1.1]heptane-2,4-dione (500.0 mg, 1.921 mmol, 1.0 eq) in EtOH (10.0 mL) was added 10% Pd/C(204.4 mg, 1.921 mmol, 1.0 eq). The reaction mixture was stirred under hydrogen atmosphere (1 atm) at 50° C. for 20 h. After cooled to room temperature, the mixture was filtered and the filtrate was concentrated in vacuum. The crude product is dissolved in 1.4-dioxane (5 mL) and HCl-dioxane solution (4 M) was added to above mixture. The solid was precipitated and filtered. The cake was washed with 1.4-dioxane and dried to obtain 1-amino-3-azabicyclo[3.1.1]heptane-2,4-dione hydrochloride (250 mg, yield 92%) as a white solid. LC-MS (ESI): mass calced for: C₆H₈N₂O₂, 140.14; m/z found, 141.1 [M+H]⁺. ¹H NMR (400 MHZ, DMSO-d₆) δ 11.24 (s, 1H), 9.13 (s, 3H), 3.08-3.04 (m, 1H), 2.88-2.84 (m, 2H), 2.58-2.52 (m, 2H).

### Step D: methyl 4-bromo-2-(((2,4-dioxo-3-azabicyclo[3.1.1]heptan-1-yl)amino)methyl)benzoate

To a solution of methyl 4-bromo-2-(bromomethyl)benzoate (233.0 mg, 756.6 μmol, 1.0 eq) in Acetonitrile (10.00 mL) were added DIPEA (391.1 mg, 3.026 mmol, 4.0 eq) and 1-amino-3-azabicyclo[3.1.1]heptane-2,4-dione HCl (200.4 mg, 1.135 mmol, 1.5 eq). The mixture was stirred at 80° C. for 16 h. After cooled to room temperature, the mixture was concentrated in vacuum to obtain crude product methyl 4-bromo-2-(((2,4-dioxo-3-azabicyclo[3.1.1]heptan-1-yl)amino)methyl)benzoate (300 mg, yield 99%) as a white solid. LC-MS (ESI): mass calced for: C₁₅H₁₅BrN₂O₄, 366.02; m/z found, 367.2 [M+H]⁺.

### Step E: 1-(5-bromo-1-oxoisoindolin-2-yl)-3-azabicyclo[3.1.1]heptane-2,4-dione

To a solution of methyl 4-bromo-2-(((2,4-dioxo-3-azabicyclo[3.1.1]heptan-1-yl)amino)methyl)benzoate (300 mg, 817 μmol, 1.0 eq) in DCM (10.0 mL) and Acetonitrile (2.00 mL) was added AcOH (245 mg, 4.08 mmol, 5.0 eq). The reaction mixture was stirred at 20° C. for 1 h. After evaporation, the residue was slurried with a mixed solution of EA and PE (10 mL, 4/1 v/v), filtered, dried to obtain product 1-(5-bromo-1-oxoisoindolin-2-yl)-3-azabicyclo[3.1.1]heptane-2,4-dione (270 mg, yield 98%) as a white solid. LC-MS (ESI): mass calced for: C₁₄H₁₁BrN₂O₃, 334.00; m/z found, 335.2 [M+H]⁺.

### Step F: 1-(1-oxo-5-(4,4,5,5-tetramethyl-1,3,2-dioxaborolan-2-yl) isoindolin-2-yl)-3-azabicyclo[3.1.1]heptane-2,4-dione

To a solution of 1-(5-bromo-1-oxoisoindolin-2-yl)-3-azabicyclo[3.1.1]heptane-2,4-dione (270.0 mg, 805.6 μmol, 1.0 eq) in 1,4-Dioxane (5.00 mL) were added Bis(pinacolato) diborane (409.1 mg, 1.611 mmol, 2.0 eq), anhydrous potassium acetate (237.2 mg, 2.417 mmol, 3.0 eq), and PdCl₂ (dppf) (58.95 mg, 80.56 μmol, 0.1 eq). The mixture was stirred under N₂ at 100° C. for 3 h. After cooled to room temperature, the mixture was concentrated under reduced pressure. The residue is dissolved in water (5 mL), filtered, and the filter cake was washed with EA (5 mL). The cake was dried to give 1-(1-oxo-5-(4,4,5,5-tetramethyl-1,3,2-dioxaborolan-2-yl) isoindolin-2-yl)-3-azabicyclo[3.1.1]heptane-2,4-dione (230 mg, yield 74%) as a gray solid. LC-MS (ESI): mass calced for: C₂₀H₂₃BN₂O₅, 382.22; m/z found, 383.2 [M+H]⁺. ¹H NMR (400 MHZ, DMSO-d₆) δ 10.97 (s, 1H), 7.89 (s, 1H), 7.81 (d, J=7.4 Hz, 1H), 7.69 (d, J=7.4 Hz, 1H), 4.41 (s, 2H), 3.08 (s, 1H), 2.85 (s, 4H), 1.33 (s, 12H).

### Intermediate 119:2-chloro-1,5-naphthyridine-4-carbaldehyde

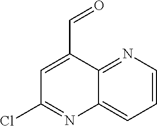 [SMILES: O=Cc1cc(Cl)nc2cccnc12]

### Step A: 1-(tert-butyl) 3-ethyl 2-(3-nitropyridin-2-yl) malonate

To a solution of t-BuOK (5.31 g, 47.3 mmol, 1.5 eq) in THF (100 mL) was added dropwise tert-butyl ethyl malonate (8.90 g, 47.3 mmol, 1.5 eq) at 60° C., follow by a solution of 2-chloro-3-nitropyridine (5.00 g, 31.5 mmol, 1.0 eq) in THF (30.0 mL). The resulting reaction mixture was stirred at 70° C. for 4 h. After cooled to room temperature, the reaction mixture was concentrated under reduced pressure and the residue was diluted with diluted aqueous HCl solution (1 M) (80 mL). The aqueous solution was extracted with EA (100 mL×4). The organic layer was washed with brine (300 mL), dried over anhydrous Na₂SO₄, filtered and concentrated under reduced pressure to give crude 1-(tert-butyl) 3-ethyl 2-(3-nitropyridin-2-yl) malonate (13 g, yield 133%) as a red oil. LC-MS (ESI): mass calcd. for C₁₄H₁₈N₂O₆, 310.31; m/z found, 311.3 [M+H]⁺. ¹H NMR (400 MHZ, CDCl₃) δ 8.82 (dd, J=4.6, 1.4 Hz, 1H), 8.47 (dd, J=8.2, 1.4 Hz, 1H), 7.51 (dd, J=8.2, 4.6 Hz, 1H), 5.44 (s, 1H), 4.31 (dddd, J=17.8, 10.8, 7.0, 3.6 Hz, 2H), 1.49 (s, 9H), 1.29 (d, J=7.2 Hz, 3H).

### Step B: ethyl 2-(3-nitropyridin-2-yl) acetate

To a solution of 1-(tert-butyl) 3-ethyl 2-(3-nitropyridin-2-yl) malonate (13 g, 41.9 mmol, 1.0 eq) in DCM (60 mL) were added dropwise TFA (20 mL). The mixture was stirred at room temperature for 16 h. After evaporation, the residue was diluted with saturated aqueous NaHCO₃ solution (50 mL) and extracted with EA (50 mL×3). The organic layer was washed with brine (80 mL), dried over anhydrous Na₂SO₄, filtered and concentrated under reduced pressure to give crude ethyl 2-(3-nitropyridin-2-yl) acetate (6.3 g, yield 71%, two steps) as a brown oil. LC-MS (ESI): mass calcd. for C₉H₁₀N₂O₄, 210.19; m/z found, 211.2 [M+H]⁺. ¹H NMR (400 MHZ, CDCl₃) δ 8.80 (dd, J=4.6, 1.4 Hz, 1H), 8.43 (dd, J=8.2, 1.2 Hz, 1H), 7.48 (dd, J=8.2, 4.6 Hz, 1H), 4.33 (s, 2H), 4.20 (q, J=7.0 Hz, 2H), 1.26 (t, J=7.0 Hz, 3H).

### Step C: ethyl 2-(3-aminopyridin-2-yl) acetate

To a solution of ethyl 2-(3-nitropyridin-2-yl) acetate (6.3 g, 30 mmol, 1.0 eq) in EtOH (60 mL) were added 10% Pd/C(630 mg). The mixture was stirred under H₂ atmosphere (1 atm) at 25° C. for 16 h. After filtration, the filtrate was concentrated under reduced pressure to give crude ethyl 2-(3-aminopyridin-2-yl) acetate (5.4 g, yield 100%) as a brown oil. LC-MS (ESI): mass calcd. for C₉H₁₂N₂O₂, 180.21; m/z found, 181.2 [M+H]⁺. ¹H NMR (400 MHZ, DMSO-d6) δ 7.74 (dd, J=3.2, 2.8 Hz, 1H), 7.07-7.01 (m, 2H), 5.24 (s, 2H), 4.08 (q, J=7.0 Hz, 2H), 3.72 (s, 2H), 1.18 (t, J=7.0 Hz, 3H).

### Step D: 2,2-diethoxyacetic acid

To a solution of ethyl 2,2-diethoxyacetate (8.0 g, 45.4 mmol, 1.0 eq) in EtOH (30 mL) were added aqueous NaOH solution (2 N) (45.4 mL, 90.8 mmol, 2.0 eq). The mixture was stirred at room temperature for 16 h. After evaporated to remove EtOH, the residue was extracted with a mixed solvent of PE/EA (10/1) and the organic layer was discarded off. The aqueous layer was acidified to pH 3˜4 with aqueous HCl solution (2 N), saturated with solid NaCl, and extracted with EA (30 mL×3). The organic layer was dried over anhydrous Na₂SO₄, filtered and concentrated under reduced pressure to give crude 2,2-diethoxyacetic acid (6.6 g, yield 98%) as a colorless oil. LC-MS (ESI): mass calcd. for C₆H₁₂O₄, 148.16; m/z found, 147.1 [M−H]⁻. ¹H NMR (400 MHZ, DMSO-d₆) δ 12.85 (s, 1H), 4.81 (s, 1H), 3.63-3.50 (m, 4H), 1.14 (t, J=7.0 Hz, 6H).

### Step E: ethyl 2-(3-(2,2-diethoxyacetamido) pyridin-2-yl) acetate

To a solution of 2,2-diethoxyacetic acid (4.88 g, 33.0 mmol, 1.1 eq) in DMF (55 mL) were added Diisopropylethylamine (11.6 g, 89.9 mmol, 3.0 eq) and HATU (17.1 g, 44.9 mmol, 1.5 eq). The mixture was stirred at room temperature for 30 minutes, ethyl 2-(3-aminopyridin-2-yl) acetate (5.40 g, 30.0 mmol, 1.0 eq) was added to above mixture. The resulting mixture was stirred at room temperature for 16 h. The reaction mixture was quenched with saturated aqueous NaHCO₃ solution (100 mL) and extracted with EA (70 mL×3). The organic layer was washed with brine (80 mL×4), dried over anhydrous Na₂SO₄, filtered and concentrated under reduced pressure. The crude product was purified by flash column chromatography on silica gel (ethyl acetate in petroleum ether, from 0% to 43% v/v) to give ethyl 2-(3-(2,2-diethoxyacetamido) pyridin-2-yl) acetate (4.3 g, yield 46%) as a yellow oil. LC-MS (ESI): mass calcd. for C₁₅H₂₂N₂O₅, 310.35; m/z found, 311.4 [M+H]⁺. ¹H NMR (400 MHz, DMSO-d₆) δ 9.59 (s, 1H), 8.35 (dd, J=4.6, 1.2 Hz, 1H), 7.87 (dd, J=8.0, 1.0 Hz, 1H), 7.35 (dd, J=8.0, 4.6 Hz, 1H), 4.95 (s, 1H), 4.08 (q, J=7.0 Hz, 2H), 3.87 (s, 2H), 3.65 (tdd, J=9.6, 7.0, 2.4 Hz, 4H), 1.22-1.15 (m, 9H).

### Step F: ethyl 2-oxo-1,2-dihydro-1,5-naphthyridine-4-carboxylate

To a solution of ethyl 2-(3-(2,2-diethoxyacetamido) pyridin-2-yl) acetate (1.0 g, 3.2 mmol, 1.0 eq) in TFA (15 mL) and water (0.5 mL) were added I₂ solution (30 mg I₂ in 10 mL of TFA) (1 drop). The mixture was stirred at 50° C. in a sealed tube for 16 h. After cooled to room temperature, the mixture was concentrated under reduced pressure to give crude ethyl 2-oxo-1,2-dihydro-1,5-naphthyridine-4-carboxylate (1.3 g, yield 185%) as a dark purple oil. LC-MS (ESI): mass calcd. for C₁₁H₁₀N₂O₃, 218.21; m/z found, 219.2 [M+H]⁺.

### Step G: ethyl 2-chloro-1,5-naphthyridine-4-carboxylate

A solution of ethyl 2-oxo-1,2-dihydro-1,5-naphthyridine-4-carboxylate (1.3 g, 5.9 mmol, 1.0 eq) in POCl₃ (15 mL) was stirred at 90° C. for 4 h. After evaporated to remove POC13, the residue was adjusted to pH-8 with saturated aqueous NaHCO₃ solution at 0° C. and extracted with DCM (20 mL×4). The organic layer was washed with brine (60 mL), dried over anhydrous Na₂SO₄, filtered and concentrated under reduced pressure. The crude product was purified by flash column chromatography on silica gel (ethyl acetate in petroleum ether, from 0% to 30% v/v) to give ethyl 2-chloro-1,5-naphthyridine-4-carboxylate (270 mg, yield 19%, two steps) as a yellow solid. LC-MS (ESI): mass calcd. for C₁₁H₉ClN₂O₂, 236.66; m/z found, 237.5 [M+H]⁺. ¹H NMR (400 MHZ, DMSO-d₆) δ 9.08 (dd, J=4.2, 1.6 Hz, 1H), 8.47 (dd, J=8.6, 1.6 Hz, 1H), 8.09 (s, 1H), 7.93 (dd, J=8.6, 4.2 Hz, 1H), 4.46 (q, J=7.0 Hz, 2H), 1.36 (t, J=7.0 Hz, 3H).

### Step H: (2-chloro-1,5-naphthyridin-4-yl) methanol

To a solution of ethyl 2-chloro-1,5-naphthyridine-4-carboxylate (270 mg, 1.1 mmol, 1 eq) in anhydrous THF (5 mL) was added DIBAL-H solution (1 Nin THF) (1.7 mL, 1.7 mmol, 1.5 eq) at −70° C. The mixture was stirred at −70° C. for 15 min, diluted with THF (20 mL), quenched with Na₂SO₄·10H₂O, and filtered. The filtrate was concentrated under reduced pressure to give (2-chloro-1,5-naphthyridin-4-yl) methanol (250 mg, yield 113%) as a yellow solid. LC-MS (ESI): mass calcd. for C₉H₇ClN₂O, 194.62; m/z found, 195.3 [M+H]⁺.

### Step I: 2-chloro-1,5-naphthyridine-4-carbaldehyde

To a solution of ethyl (2-chloro-1,5-naphthyridin-4-yl) methanol (250 mg, 1.2 mmol, 1.0 eq) in DCM (10 mL) were added Dess-Martin Periodinane (817 mg, 1.9 mmol, 1.5 eq) at 0° C. The mixture was stirred at room temperature for 1 h. The reaction mixture was quenched with aqueous NaHSO₃ solution (20 mL) and extracted with DCM (20 mL×3). The organic layer was washed with brine (40 mL), dried over anhydrous Na₂SO₄, filtered and concentrated under reduced pressure. The crude product was purified by flash column chromatography on silica gel (ethyl acetate in petroleum ether, from 0% to 40% v/v) to give 2-chloro-1,5-naphthyridine-4-carbaldehyde (80 mg, yield 32%) as a yellow solid. LC-MS (ESI): mass calcd. for C₉H₅ClN₂O, 192.6; m/z found, 193.4 [M+H]⁺. ¹H NMR (400 MHZ, DMSO-d₆) δ 11.20 (s, 1H), 9.22 (dd, J=4.2, 1.6 Hz, 1H), 8.55 (dd, J=8.6, 1.4 Hz, 1H), 8.00 (dd, J=9.0, 3.6 Hz, 2H).

### Intermediate 120:6-chloro-1-isopropyl-1H-pyrrolo[2,3-b]pyridine-4-carbaldehyde

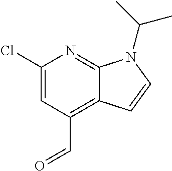

The title compound was obtained simlart to the procedure in Intermediate 10 by alkylation of methyl 6-chloro-1H-pyrrolo[2,3-b]pyridine-4-carboxylate with iso-propyl iodide by NaH followed by LAH reduction and IBX oxidation. LC-MS (ESI): mass calcd. for C₁H₁₁ClN₂O, 222.1; m/z found, 223.4 [M+H]⁺.

### Intermediate 121a and 121b: 5-chloro-1-methyl-1H-pyrazolo[4,3-b]pyridine-7-carbaldehyde (121a) and 5-chloro-2-methyl-2H-pyrazolo[4,3-b]pyridine-7-carbaldehyde (121b)

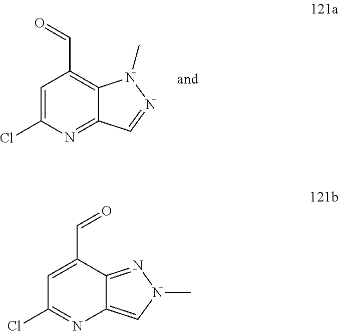

### Step A: ethyl 5-chloro-1-methyl-1H-pyrazolo[4,3-b]pyridine-7-carboxylate and ethyl 5-chloro-2-methyl-2H-pyrazolo[4,3-b]pyridine-7-carboxylate

To a solution of ethyl 5-chloro-1H-pyrazolo[4,3-b]pyridine-7-carboxylate (1.10 g, 4.88 mmol) in DMF (20.0 mL) was added Cs₂CO₃ (3.18 g, 9.75 mmol) and the mixture was stirring at 0° C. for 10 min. Then the mixture was added with iodomethane (2.08 g, 14.6 mmol) and the mixture was stirring at room temperature for 1 hour. The residue was poured into water (20 mL) and extracted with ethyl acetate (20 mL). The organic layer was dried over MgSO₄, filtered and concentrated. The organic layers were washed with brine, dried (Na₂SO₄), filtered and concentrated. The crude was purified by column chromatography on silica gel (10% EA/PE) to give the desired compound ethyl 5-chloro-1-methyl-1H-pyrazolo[4,3-b]pyridine-7-carboxylate (700 mg, 54%) and ethyl 5-chloro-2-methyl-2H-pyrazolo[4,3-b]pyridine-7-carboxylate (230 mg, 18%) as a white solid. LC-MS (ESI): mass calcd. for C₁₀H₁₀ClN₃O₂, 239.1; m/z found, 240.1 [M+H]⁺.

### Step B: (5-chloro-1-methyl-1H-pyrazolo[4,3-b]pyridin-7-yl) methanol

To a solution of ethyl 5-chloro-1-methyl-1H-pyrazolo[4,3-b]pyridine-7-carboxylate (700 mg, 2.92 mmol) in THF (10.0 mL) was added DIBAL-H (623 mg, 4.38 mmol) dropwise under N₂ with stirring at −78° C. for 10 min. Then the mixture was warmed to 0° C. and stirring for 1 hours. The reaction mixture was warmed to room temperature and added H₂O (5 mL). The residue was poured into water (20 mL) and extracted with EtOAc (20 mL). The organic layer was dried over MgSO₄, filtered and concentrated and purified by silica gel column chromatography (PE: ethyl acetate=2:1) to obtain (5-chloro-1-methyl-1H-pyrazolo[4,3-b]pyridin-7-yl) methanol (300 mg, 47%) as a yellow solid. LC-MS (ESI): mass calcd. for C₈H&ClN₃O, 197.0; m/z found, 198.1 [M+H]⁺.

### Step C: 5-chloro-1-methyl-1H-pyrazolo[4,3-b]pyridine-7-carbaldehyde (12-la) and 5-chloro-2-methyl-2H-pyrazolo[4,3-b]pyridine-7-carbaldehyde (121b)

To a solution of (5-chloro-1-methyl-1H-pyrazolo[4,3-b]pyridin-7-yl) methanol (200 mg, 1.01 mmol) in DMSO (5.00 mL) was added IBX (708 mg, 2.53 mmol) and the mixture was stirred at room temperature for 1 h. The mixture was quenched with Na₂CO₃ (aq.) and poured into water (6 mL) and extracted with EA (10 mL×3). The organic layer was dried over MgSO₄, filtered and concentrated under reduced pressure. The residue was purified by Prep-TLC(EA/PE=1/1) to give 5-chloro-1-methyl-1H-pyrazolo[4,3-b]pyridine-7-carbaldehyde (100 mg, 45%) as a yellow solid. LC-MS (ESI): mass calcd. for C₈H₆ClN₃O, 195.2; m/z found, 196.1 [M+H]⁺.

5-chloro-2-methyl-2H-pyrazolo[4,3-b]pyridine-7-carbaldehyde (121b) was obtained via the same reaction sequence and protocol. LC-MS (ESI): mass calcd. for C₈H₆ClN₃O, 195.2; m/z found, 196.1 [M+H]⁺.

### Intermediate 122a and 122b: 6-chloro-1-methyl-1H-pyrazolo[3,4-b]pyridine-4-carbaldehyde (122a) and 6-chloro-2-methyl-2H-pyrazolo[3,4-b]pyridine-4-carbaldehyde (122b)

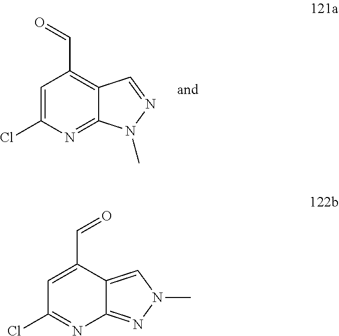

The title compound was obtained simlar to the procedure in Intermediate 121a and 121b by methylation of ethyl 6-chloro-1H-pyrazolo[3,4-b]pyridine-4-carboxylate followed by DIBAL reduction and IBX oxidation. LC-MS (ESI): mass calcd. for C₈H₆ClN₃O, 195.2; m/z found, 196.1 [M+H]⁺.

### Intermediate 123:1-bromoimidazo[1,5-a]pyridine-7-carbaldehyde

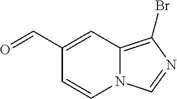 [SMILES: O=Cc1ccn2cnc(Br)c2c1]

The title compound was obtained simlar to the procedure in Intermediate 2 by DIBAL reduction of ethyl 1-bromoimidazo[1,5-a]pyridine-7-carboxylate (commericailly available) followed by Dess-Martin oxidation. LC-MS (ESI): mass calcd. for C₈H₅BrN₂O, 223.9; m/z found, 225.1 [M+H]⁺.

### Example 1. 3-(1-oxo-5-(7-(pyrrolidin-1-ylmethyl) imidazo[1,5-a]pyridin-5-yl) isoindolin-2-yl) piperidine-2,6-dione

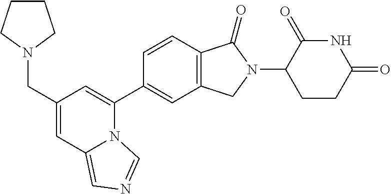 [SMILES: O=C1CCC(N2Cc3cc(-c4cc(CN5CCCC5)cc5cncn45)ccc3C2=O)C(=O)N1]

### Step A: 1-({5-chloroimidazo[1,5-a]pyridin-7-yl}methyl) pyrrolidine

To a solution of 5-chloroimidazo[1,5-a]pyridine-7-carbaldehyde (Intermediate 1, 50 mg, 0.277 mmol, 1.0 eq) and pyrrolidine (0.034 mL, 0.415 mmol, 1.5 eq) in DCM (1 mL) was added AcOH (0.1 mL) and the mixture was stirring at room temperature for 1 h. Then NaBH(OAc) 3 (176.04 mg, 0.831 mmol, 3.0 eq) was added to above mixture and the mixture was stirred at room temperature overnight. The residue was poured into water (6 mL) and extracted with DCM (10 mL×3). The organiclayer was dried over MgSO₄, filtered and concentrated under reduced pressure. The crude product was purified by Prep-TLC (DCM/MeOH=10/1) to give compound 1-({5-chloroimidazo[1,5-a]pyridin-7-yl}methyl) pyrrolidine (40 mg, yield 55%) as a white solid.

LC-MS (ESI): mass calcd. for C₁₂H₁₄ClN₃, 235.09; m/z found, 236.3 [M+H]⁺.

### Step B: 3-(1-oxo-5-{7-[(pyrrolidin-1-yl)methyl]imidazo[1,5-a]pyridin-5-yl}-2,3-dihydro-1H-isoindol-2-yl) piperidine-2,6-dione

To a solution of 5-chloroimidazo[1,5-a]pyridine-7-carbaldehyde (40 mg, 0.221 mmol, 1.0 eq) and 3-(1-oxo-5-(4,4,5,5-tetramethyl-1,3,2-dioxaborolan-2-yl) isoindolin-2-yl) piperidine-2,6-dione (Intermediate 13, 75.39 mg, 0.204 mmol, 1.2 eq) in dioxane (1 mL) and water (0.1 mL) were added Pd (dtbpf) Cl₂ (5 mg, 0.02 mmol, 0.2 eq) and K₃PO₄ (60.12 mg, 0.286 mmol, 3.0 eq). The mixture was stirred under N₂ at 95° C. for 2 hours. The reaction mixture was cooled to room temperature and concentrated under reduced pressure. The residue was poured into water (5 mL) and extracted with EtOAc (10 mL×3). The organic layer was dried over MgSO₄, filtered and concentrated under reduced pressure. The residue was purified by Prep-TLC(DCM/MeOH=10/1) to obtain 3-(1-oxo-5-{7-[(pyrrolidin-1-yl)methyl]imidazo[1,5-a]pyridin-5-yl}-2,3-dihydro-1H-isoindol-2-yl) piperidine-2,6-dione (5 mg, yield 6.3%) as a yellow solid.

LC-MS (ESI): mass calcd. for C₂₅H₂₅N₅O₃, 443.20; m/z found, 444.20 [M+H]⁺. ¹H NMR (400 MHZ, DMSO-d₆) δ 11.02 (s, 1H), 8.30 (s, 1H), 8.02 (s, 1H), 7.92-7.85 (m, 2H), 7.50 (s, 1H), 7.45 (s, 1H), 6.71 (s, 1H), 5.19-5.14 (m, 1H), 4.57 (d, J=17.6 Hz, 1H), 4.45 (d, J=17.6 Hz, 1H), 3.57 (s, 2H), 3.42 (s, 4H), 2.96-2.85 (m, 1H), 2.64-2.60 (m, 1H), 2.44-2.37 (m, 1H), 2.06-2.02 (m, 1H), 1.71 (s, 4H).

### Example 2. 3-(5-(7-(azetidin-1-ylmethyl) imidazo[1,5-a]pyridin-5-yl)-1-oxoisoindolin-2-yl) piperidine-2,6-dione

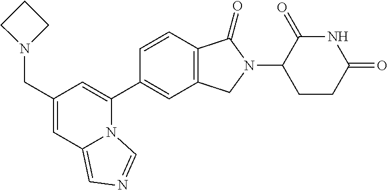 [SMILES: O=C1CCC(N2Cc3cc(-c4cc(CN5CCC5)cc5cncn45)ccc3C2=O)C(=O)N1]

The title compound was prepared in a manner analogous to Example 1 by reductive amination between azetidine and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₄H₂₃N₅O₃, 429.18; m/z found, 430.18 [M+H]⁺. ¹H NMR (400 MHZ, DMSO-d₆) δ 11.02 (s, 1H), 8.31 (s, 1H), 8.04 (d, J=20.2 Hz, 1H), 7.93-7.85 (m, 2H), 7.47 (s, 2H), 6.67 (s, 1H), 5.19-5.14 (m, 1H), 4.54 (d, J=17.6 Hz, 1H), 4.47 (d, J=17.6 Hz, 1H), 3.50 (s, 2H), 3.23-3.21 (m, 2H), 2.56 (s, 2H), 2.46-2.41 (m, 3H), 2.08-2.04 (m, 3H).

### Example 3. 3-(1-oxo-5-(7-(piperidin-1-ylmethyl) imidazo[1,5-a]pyridin-5-yl) isoindolin-2-yl) piperidine-2,6-dione

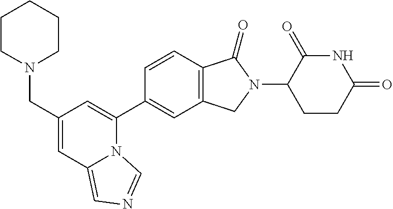 [SMILES: O=C1CCC(N2Cc3cc(-c4cc(CN5CCCCC5)cc5cncn45)ccc3C2=O)C(=O)N1]

The title compound was prepared in a manner analogous to Example 1 by reductive amination between piperdine and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₆H₂₇N₅O₃, 457.21; m/z found, 458.21 [M+H]⁺. ¹H NMR (400 MHZ, DMSO-d₆) δ 11.02 (s, 1H), 8.30 (s, 1H), 8.01 (s, 1H), 7.92-7.85 (m, 2H), 7.47 (d, J=12.2 Hz, 2H), 6.69 (s, 1H), 5.19-5.14 (m, 1H), 4.57 (d, J=17.6 Hz, 1H), 4.46 (d, J=17.6 Hz, 1H), 3.66 (s, 2H), 2.93-2.90 (m, 1H), 2.66-2.59 (m, 1H), 2.43 (s, 1H), 2.38 (s, 4H), 2.04 (s, 1H), 1.50 (d, J=5.0 Hz, 4H), 1.39 (s, 2H).

### Example 4. 3-(5-(7-(morpholinomethyl) imidazo[1,5-a]pyridin-5-yl)-1-oxoisoindolin-2-yl) piperidine-2,6-dione

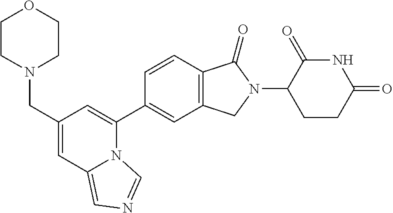 [SMILES: O=C1CCC(N2Cc3cc(-c4cc(CN5CCOCC5)cc5cncn45)ccc3C2=O)C(=O)N1]

The title compound was prepared in a manner analogous to Example 1 by reductive amination between morpholine and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₅H₂₅N₅O₄, 459.19; m/z found, 460.19 [M+H]⁺. ¹H NMR (400 MHZ, DMSO-d₆) δ 11.02 (s, 1H), 8.31 (s, 1H), 8.02 (s, 1H), 7.89 (dd, J=21.8, 7.7 Hz, 2H), 7.50 (d, J=20.8 Hz, 2H), 6.72 (s, 1H), 5.19-5.14 (m, 1H), 4.57 (d, J=17.6 Hz, 1H), 4.46 (d, J=17.6 Hz, 1H), 3.59 (s, 4H), 3.48 (s, 2H), 2.98-2.88 (m, 1H), 2.66-2.60 (m, 1H), 2.43 (s, 5H), 2.09-2.01 (m, 1H).

### Example 5. 3-(5-(7-((3-methylpiperidin-1-yl)methyl) imidazo[1,5-a]pyridin-5-yl)-1-oxoisoindolin-2-yl) piperidine-2,6-dione

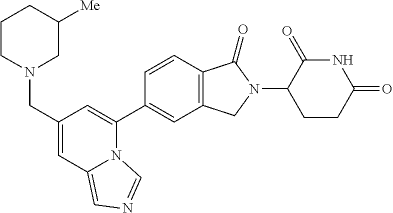 [SMILES: CC1CCCN(Cc2cc(-c3ccc4c(c3)CN(C3CCC(=O)NC3=O)C4=O)n3cncc3c2)C1]

The title compound was prepared in a manner analogous to Example 1 by reductive amination between 3-methylpiperidine and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₇H₂₉N₅O₃, 471.23; m/z found, 472.23 [M+H]⁺. ¹HNMR (400 MHZ, DMSO-d₆) δ 11.02 (s, 1H), 8.31 (s, 1H), 8.02 (s, 1H), 7.92 (d, J=7.8 Hz, 1H), 7.86 (d, J=7.8 Hz, 1H), 7.47 (d, J=11.4 Hz, 2H), 6.69 (d, J=1.2 Hz, 1H), 5.20-5.15 (m, 1H), 4.57 (d, J=17.6 Hz, 1H), 4.46 (d, J=17.6 Hz, 1H), 3.42 (s, 2H), 2.98-2.89 (m, 1H), 2.77 (t, J=9.4 Hz, 2H), 2.62 (d, J=17.5 Hz, 1H), 2.48-2.37 (m, 2H), 2.07-2.00 (m, 1H), 1.91 (t, J=10.3 Hz, 1H), 1.62 (dd, J=18.6, 10.0 Hz, 4H), 1.47 (d, J=12.2 Hz, 1H), 0.82 (d, J=6.0 Hz, 3H).

### Example 6. 3-(5-(7-((3-methylpyrrolidin-1-yl)methyl) imidazo[1,5-a]pyridin-5-yl)-1-oxoisoindolin-2-yl) piperidine-2,6-dione

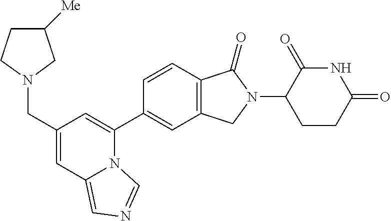 [SMILES: CC1CCN(Cc2cc(-c3ccc4c(c3)CN(C3CCC(=O)NC3=O)C4=O)n3cncc3c2)C1]

The title compound was prepared in a manner analogous to Example 1 by reductive amination between 3-methylpyrrolidine and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₆H₂₇N₅O₃, 457.21; m/z found, 458.21 [M+H]⁺. ¹H NMR (400 MHz, DMSO-d₆) δ 11.03 (s, 1H), 8.35 (s, 1H), 8.04 (s, 1H), 7.93 (d, J=7.8 Hz, 1H), 7.88 (d, J=7.6 Hz, 1H), 7.55 (d, J=28.0 Hz, 2H), 6.78 (s, 1H), 5.20-5.15 (m, 1H), 4.57 (d, J=17.6 Hz, 1H), 4.46 (d, J=17.6 Hz, 1H), 3.83 (s, 2H), 2.93 (dd, J=21.8, 9.1 Hz, 2H), 2.62 (d, J=15.5 Hz, 1H), 2.44 (d, J=4.0 Hz, 2H), 2.22 (s, 2H), 2.07-2.02 (m, 3H), 1.34-1.32 (m, 1H), 1.01 (d, J=6.4 Hz, 3H).

### Example 7. 3-(5-(7-((3-azabicyclo[3.1.0]hexan-3-yl)methyl) imidazo[1,5-a]pyridin-5-yl)-1-oxoisoindolin-2-yl) piperidine-2,6-dione

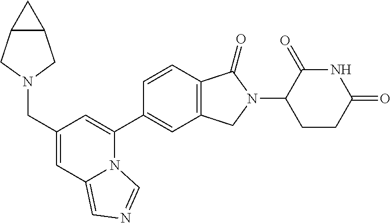 [SMILES: O=C1CCC(N2Cc3cc(-c4cc(CN5CC6CC6C5)cc5cncn45)ccc3C2=O)C(=O)N1]

The title compound was prepared in a manner analogous to Example 1 by reductive amination between 3-azabicyclo[3.1.0]hexane and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₆H₂₅N₅O₃, 455.20; m/z found, 456.20 [M+H]⁺. ¹HNMR (400 MHZ, DMSO-d₆) δ 11.02 (s, 1H), 8.30 (s, 1H), 8.01 (s, 1H), 7.92 (d, J=7.8 Hz, 1H), 7.86 (d, J=8.0 Hz, 1H), 7.45 (s, 2H), 6.64 (s, 1H), 5.20-5.15 (m, 1H), 4.57 (d, J=17.6 Hz, 1H), 4.46 (d, J=17.5 Hz, 1H), 3.57 (s, 2H), 2.94-2.91 (m, 3H), 2.62 (d, J=17.9 Hz, 1H), 2.44 (s, 1H), 2.37 (d, J=8.3 Hz, 2H), 2.07-2.01 (m, 1H), 1.37 (s, 2H), 0.69 (d, J=3.7 Hz, 1H), 0.34-0.31 (m, 1H).

### Example 8. 3-(5-(7-((6-azaspiro[2.5]octan-6-yl)methyl) imidazo[1,5-a]pyridin-5-yl)-1-oxoisoindolin-2-yl) piperidine-2,6-dione

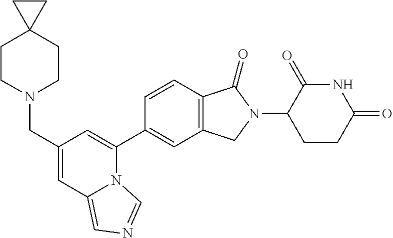 [SMILES: O=C1CCC(N2Cc3cc(-c4cc(CN5CCC6(CC5)CC6)cc5cncn45)ccc3C2=O)C(=O)N1]

The title compound was prepared in a manner analogous to Example 1 by reductive amination between 6-azaspiro[2.5]octane and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₈H₂₉N₅O₃, 483.23; m/z found, 484.23 [M+H]⁺. ¹H NMR (400 MHZ, DMSO) δ 11.02 (s, 1H), 8.31 (s, 1H), 8.02 (s, 1H), 7.92 (d, J=8.0 Hz, 1H), 7.87 (d, J=8.2 Hz, 1H), 7.50 (s, 1H), 7.46 (s, 1H), 6.72 (s, 1H), 5.19-5.14 (m, 1H), 4.58 (d, J=17.6 Hz, 1H), 4.46 (d, J=17.6 Hz, 1H), 3.49 (s, 2H), 2.94-2.91 (m, 1H), 2.62 (d, J=16.0 Hz, 1H), 2.44-2.41 (m, 5H), 2.06-2.02 (m, 1H), 1.35-1.34 (m, 4H), 0.25 (s, 4H).

### Example 9. 3-(5-(7-((7-azaspiro[3.5]nonan-7-yl)methyl) imidazo[1,5-a]pyridin-5-yl)-1-oxoisoindolin-2-yl) piperidine-2,6-dione

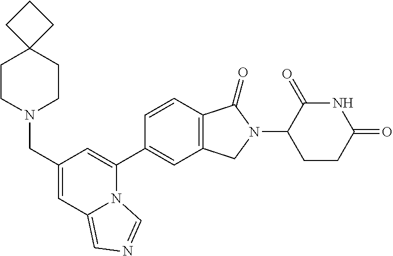 [SMILES: O=C1CCC(N2Cc3cc(-c4cc(CN5CCC6(CCC6)CC5)cc5cncn45)ccc3C2=O)C(=O)N1]

The title compound was prepared in a manner analogous to Example 1 by reductive amination n between 7-azaspiro[3.5]nonane and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₉H₃₁N₅O₃, 497.24; m/z found, 498.25 [M+H]⁺. ¹H NMR (400 MHZ, DMSO-d₆) δ 11.02 (s, 1H), 8.31 (s, 1H), 8.07 (s, 1H), 8.02 (s, 1H), 7.92 (d, J=7.8 Hz, 1H), 7.86 (d, J=9.0 Hz, 1H), 7.47 (d, J=11.4 Hz, 2H), 6.70 (s, 1H), 5.20-5.15 (m, 1H), 4.57 (d, J=17.6 Hz, 1H), 4.46 (d, J=17.6 Hz, 1H), 3.42 (s, 2H), 2.95-2.89 (m, 1H), 2.62 (d, J=18.6 Hz, 1H), 2.44 (s, 1H), 2.31-2.25 (m, 2H), 2.18 (t, J=7.3 Hz, 1H), 2.05-1.98 (m, 2H), 1.82 (d, J=7.7 Hz, 2H), 1.70 (t, J=7.2 Hz, 4H), 1.55 (s, 4H).

### Example 10. 3-(5-(7-((2-oxa-7-azaspiro[3.5]nonan-7-yl)methyl) imidazo[1,5-a]pyridin-5-yl)-1-oxoisoindolin-2-yl) piperidine-2,6-dione

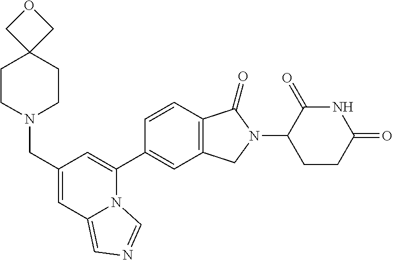 [SMILES: O=C1CCC(N2Cc3cc(-c4cc(CN5CCC6(CC5)COC6)cc5cncn45)ccc3C2=O)C(=O)N1]

The title compound was prepared in a manner analogous to Example 1 by reductive amination between 2-oxa-7-azaspiro[3.5]nonane and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₈H₂₉N₅O₄, 499.22; m/z found, 500.22 [M+H]⁺. ¹H NMR (400 MHZ, DMSO-d₆) δ 11.03 (s, 1H), 8.31 (s, 1H), 8.17 (s, 1H), 8.00 (s, 1H), 7.91 (d, J=7.9 Hz, 1H), 7.85 (d, J=7.9 Hz, 1H), 7.46 (d, J=7.8 Hz, 2H), 6.68 (s, 1H), 5.19-5.14 (m, 1H), 4.56 (d, J=17.6 Hz, 1H), 4.45 (d, J=17.6 Hz, 1H), 4.26 (s, 4H), 3.41 (s, 2H), 2.98-2.88 (m, 1H), 2.67-2.56 (m, 1H), 2.47-2.40 (m, 1H), 2.32 (s, 4H), 2.07-2.02 (m, 1H), 1.77 (s, 4H).

### Example 11. 3-(5-(7-((3-oxa-7-azabicyclo[3.3.1]nonan-7-yl)methyl) imidazo[1,5-alpyridin-5-yl)-1-oxoisoindolin-2-yl) piperidine-2,6-dione

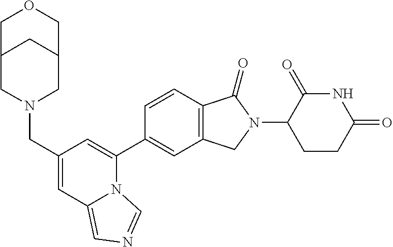 [SMILES: O=C1CCC(N2Cc3cc(-c4cc(CN5CC6COCC(C6)C5)cc5cncn45)ccc3C2=O)C(=O)N1]

The title compound was prepared in a manner analogous to Example 1 by reductive amination between 3-oxa-7-azabicyclo[3.3.1]nonane and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₈H₂₉N₅O₄, 499.22; m/z found, 500.4 [M+H]⁺. ¹HNMR (400 MHZ, DMSO-d₆) δ 11.02 (s, 1H), 8.32 (s, 1H), 8.02 (s, 1H), 7.93 (d, J=7.8 Hz, 1H), 7.85 (d, J=8.0 Hz, 1H), 7.58 (s, 1H), 7.48 (s, 1H), 6.84 (s, 1H), 5.20-5.15 (m, 1H), 4.58 (d, J=17.4 Hz, 1H), 4.46 (d, J=17.4 Hz, 1H), 3.79 (d, J=11.2 Hz, 2H), 3.64 (d, J=10.2 Hz, 3H), 3.51 (s, 1H), 2.97-2.90 (m, 4H), 2.62 (d, J=16.8 Hz, 1H), 2.44 (s, 2H), 2.03 (s, 1H), 1.76 (s, 3H), 1.61 (s, 1H).

### Example 12. 3-(5-(7-((diethylamino)methyl) imidazo[1,5-a]pyridin-5-yl)-1-oxoisoindolin-2-yl) piperidine-2,6-dione

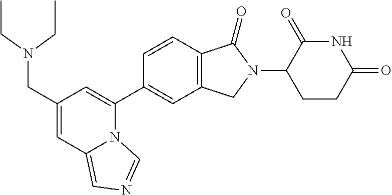 [SMILES: CCN(CC)Cc1cc(-c2ccc3c(c2)CN(C2CCC(=O)NC2=O)C3=O)n2cncc2c1]

The title compound was prepared in a manner analogous to Example 1 by reductive amination between diethylamine and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₅H₂₇N₅O₃, 445.21; m/z found, 446.21 [M+H]⁺. ¹H NMR (400 MHZ, DMSO-d₆) δ 11.03 (s, 1H), 8.30 (s, 1H), 8.01 (s, 1H), 7.92 (d, J=7.8 Hz, 1H), 7.86 (d, J=8.2 Hz, 1H), 7.53 (s, 1H), 7.46 (s, 1H), 6.71 (s, 1H), 5.20-5.15 (m, 1H), 4.58 (d, J=17.6 Hz, 1H), 4.46 (d, J=17.6 Hz, 1H), 3.56 (s, 2H), 2.96-2.88 (m, 1H), 2.65-2.53 (m, 5H), 2.46-2.44 (m, 1H), 2.08-2.01 (m, 1H), 1.01 (t, J=7.0 Hz, 6H).

### Example 13. 3-(5-(7-((benzylamino)methyl) imidazo[1,5-a]pyridin-5-yl)-1-oxoisoindolin-2-yl) piperidine-2,6-dione

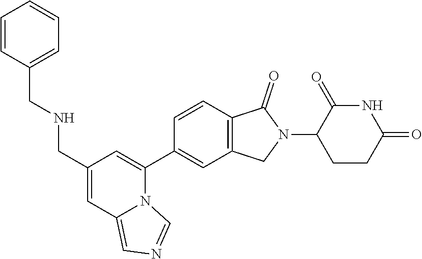 [SMILES: O=C1CCC(N2Cc3cc(-c4cc(CNCc5ccccc5)cc5cncn45)ccc3C2=O)C(=O)N1]

The title compound was prepared in a manner analogous to Example 1 by reductive amination between benzylamine and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₈H₂₅N₅O₃, 479.20; m/z found, 480.20 [M+H]⁺. ¹H NMR (400 MHZ, DMSO-d₆) δ 11.03 (s, 1H), 8.31 (s, 1H), 8.02 (s, 1H), 7.93 (d, J=7.8 Hz, 1H), 7.87 (d, J=8.0 Hz, 1H), 7.54 (s, 1H), 7.46 (s, 1H), 7.40-7.30 (m, 4H), 7.24 (t, J=7.2 Hz, 1H), 6.81 (s, 1H), 5.20-5.16 (m, 1H), 4.58 (d, J=17.6 Hz, 1H), 4.46 (d, J=17.6 Hz, 1H), 3.75 (d, J=13.6 Hz, 4H), 2.99-2.89 (m, 1H), 2.65-2.60 (m, 1H), 2.48-2.43 (m, 1H), 2.08-2.02 (m, 1H).

### Example 14. 3-(1-oxo-5-(7-((((S*)-1-phenylethyl)amino)methyl) imidazo[1,5-a]pyridin-5-yl) isoindolin-2-yl) piperidine-2,6-dione

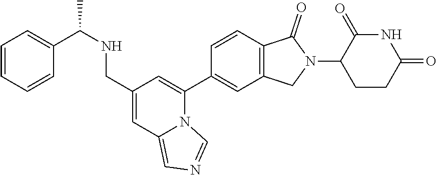 [SMILES: C[C@H](NCc1cc(-c2ccc3c(c2)CN(C2CCC(=O)NC2=O)C3=O)n2cncc2c1)c1ccccc1]

The title compound was prepared in a manner analogous to Example 1 by reductive amination between(S)-1-phenylethan-1-amine and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₉H₂₇N₅O₃, 493.21; m/z found, 494.21 [M+H]⁺. ¹H NMR (400 MHZ, DMSO-d₆) δ 11.03 (s, 1H), 8.30 (s, 1H), 8.00 (s, 1H), 7.92 (d, J=7.8 Hz, 1H), 7.86 (d, J=8.0 Hz, 1H), 7.46 (d, J=5.6 Hz, 2H), 7.39 (d, J=7.2 Hz, 2H), 7.33 (t, J=7.4 Hz, 2H), 7.24 (d, J=6.7 Hz, 1H), 6.74 (s, 1H), 5.20-5.16 (m, 1H), 4.57 (d, J=17.6 Hz, 1H), 4.46 (d, J=17.6 Hz, 1H), 3.80 (s, 1H), 3.55 (s, 2H), 2.98-2.90 (m, 1H), 2.62 (d, J=16.7 Hz, 1H), 2.47-2.43 (m, 1H), 2.08-2.01 (m, 1H), 1.32 (d, J=5.4 Hz, 3H).

### Example 15. 3-(5-(7-((methyl ((S*)-1-phenylethyl)amino)methyl) imidazo[1,5-a]pyridin-5-yl)-1-oxoisoindolin-2-yl) piperidine-2,6-dione

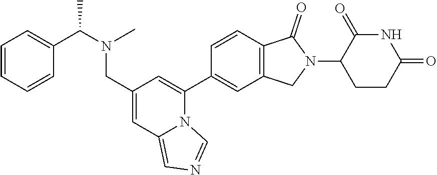 [SMILES: C[C@@H](c1ccccc1)N(C)Cc1cc(-c2ccc3c(c2)CN(C2CCC(=O)NC2=O)C3=O)n2cncc2c1]

The title compound was prepared in a manner analogous to Example 1 by reductive amination between methyl [(1S)-1-phenylethyl]amine and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13). LC-MS (ESI): mass calcd. for C₃₀H₂₉N₅O₃, 507.23; m/z found, 508.23 [M+H]⁺. ¹H NMR (400 MHz, DMSO-d₆) δ 11.04 (s, 1H), 8.28 (s, 1H), 8.00 (s, 1H), 7.93 (d, J=7.6 Hz, 1H), 7.85 (d, J=7.0 Hz, 1H), 7.48 (d, J=18.6 Hz, 2H), 7.42-7.32 (m, 4H), 7.25 (d, J=6.8 Hz, 1H), 6.66 (s, 1H), 5.20-5.16 (m, 1H), 4.59 (d, J=17.6 Hz, 1H), 4.47 (d, J=17.6 Hz, 1H), 3.73 (d, J=5.8 Hz, 1H), 3.49 (d, J=13.6 Hz, 2H), 2.95 (t, J=12.8 Hz, 1H), 2.65-2.61 (m, 1H), 2.45 (s, 1H), 2.11 (s, 3H), 2.08-2.04 (m, 1H), 1.39 (d, J=6.4 Hz, 3H).

### Example 16. 3-(1-oxo-5-(7-((((R*)-1-phenylethyl)amino)methyl) imidazo[1,5-a]pyridin-5-yl) isoindolin-2-yl) piperidine-2,6-dione

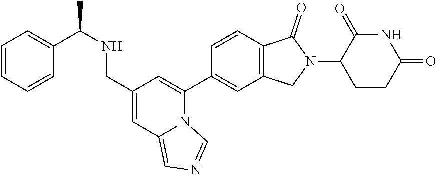 [SMILES: C[C@@H](NCc1cc(-c2ccc3c(c2)CN(C2CCC(=O)NC2=O)C3=O)n2cncc2c1)c1ccccc1]

The title compound was prepared in a manner analogous to Example 1 by reductive amination between (R)-1-phenylethan-1-amine and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₉H₂₇N₅O₃, 493.21; m/z found, 494.21 [M+H]⁺. ¹H NMR (400 MHZ, DMSO-d₆) δ 11.02 (s, 1H), 8.29 (s, 1H), 8.00 (s, 1H), 7.92 (d, J=7.8 Hz, 1H), 7.85 (d, J=7.8 Hz, 1H), 7.44 (s, 2H), 7.39-7.30 (m, 4H), 7.23 (d, J=7.0 Hz, 1H), 6.73 (s, 1H), 5.20-5.15 (m, 1H), 4.57 (d, J=17.6 Hz, 1H), 4.45 (d, J=17.6 Hz, 1H), 3.75 (d, J=15.0 Hz, 1H), 3.54 (s, 2H), 2.95-2.94 (m, 1H), 2.62 (d, J=16.0 Hz, 1H), 2.45-2.39 (m, 1H), 2.06-2.03 (m, 1H), 1.31 (d, J=6.4 Hz, 3H).

### Example 17. 3-(5-(7-((methyl ((R*)-1-phenylethyl)amino)methyl) imidazo[1,5-a]pyridin-5-yl)-1-oxoisoindolin-2-yl) piperidine-2,6-dione

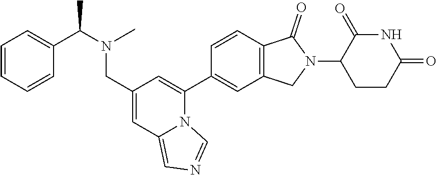 [SMILES: C[C@H](c1ccccc1)N(C)Cc1cc(-c2ccc3c(c2)CN(C2CCC(=O)NC2=O)C3=O)n2cncc2c1]

### Step A: ({5-chloroimidazo[1,5-a]pyridin-7-yl}methyl)[(1R)-1-phenylethyl]amine

To a solution of 5-chloroimidazo[1,5-a]pyridine-7-carbaldehyde (Intermediate 1, 50 mg, 0.277 mmol, 1.0 eq) and (R)-1-phenylethan-1-amine (0.035 mL, 0.277 mmol, 1.0 eq) in DMF (1 mL) was added HOAc (0.1 mL) and the mixture was stirring at room temperature for 1 h. Then NaBH₃CN (13.08 mg, 0.387 mmol, 1.4 eq) and the mixture was stirring at room temperature overnight. The residue was poured into water (6 mL) and extracted with DCM (10 mL×3). The organic layer was dried over anhydrous Na₂SO₄, filtered and concentrated under reduced pressure. The crude product was purified by Prep-TLC (DCM/MeOH=10/1) to give ({5-chloroimidazo[1,5-a]pyridin-7-yl}methyl) [(1R)-1-phenylethyl]amine (40 mg, yield 45%) as a yellow oil. LC-MS (ESI): mass calcd. for C₁₆H₁₆ClN₃, 285.10; m/z found, 286.4 [M+H]⁺.

### Step B: ({5-chloroimidazo[1,5-a]pyridin-7-yl}methyl) (methyl) [(1R)-1-phenylethyl]amine

To a solution of ({5-chloroimidazo[1,5-a]pyridin-7-yl}methyl) [(1R)-1-phenylethyl]amine (40 mg, 0.140 mmol, 1.0 eq) and aq. HCHO solution (37%) (6.30 mg, 0.210 mmol, 1.5 eq) in DMF (1 mL) was added HOAc (0.1 mL) and the mixture was stirring at room temperature for 1 h. Then NaBH₃CN (13.08 mg, 0.387 mmol, 1.4 eq) and the mixture was stirring at room temperature overnight. The residue was poured into water (6 mL) and extracted with DCM (10 mL×3). The organiclayer was dried over anhydrous Na₂SO₄, filtered and concentrated under reduced pressure. The crude product was purified by Prep-TLC (DCM/MeOH=10/1) to give ({5-chloroimidazo[1,5-a]pyridin-7-yl}methyl) (methyl) [(1R)-1-phenylethyllamine (30 mg, yield 64%) as a yellow oil. LC-MS (ESI): mass calcd. for C₁₇H₁₈ClN₃, 299.12; m/z found, 300.4 [M+H]⁺.

### Step C: 3-(5-(7-((methyl ((R*)-1-phenylethyl)amino)methyl) imidazo[1,5-a]pyridin-5-yl)-1-oxoisoindolin-2-yl) piperidine-2,6-dione

To a solution of ({5-chloroimidazo[1,5-a]pyridin-7-yl}methyl) (methyl) [(1R)-1-phenylethyl]amine (30 mg, 0.100 mmol, 1.0 eq) and 3-(1-oxo-5-(4,4,5,5-tetramethyl-1,3,2-dioxaborolan-2-yl) isoindolin-2-yl) piperidine-2,6-dione (Intermediate 13, 37.04 mg, 0.100 mmol, 1.0 eq) in dioxane (1 mL) and water (0.1 mL) were added Pd (dtbpf) Cl₂ (6.46 mg, 0.010 mmol, 0.1 eq) and K₃PO₄ (63.72 mg, 0.300 mmol, 3.0 eq). The mixture was stirred under N₂ at 95° C. for 2 hours. The reaction mixture was cooled to room temperature and concentrated under reduced pressure. The residue was poured into water (5 mL) and extracted with EtOAc (10 mL×3). The organic layer was dried over MgSO₄, filtered and concentrated under reduced pressure. The residue was purified by Prep-TLC(DCM/MeOH=10/1) to obtain 3-(5-(7-((methyl ((R*)-1-phenylethyl)amino)methyl) imidazo[1,5-a]pyridin-5-yl)-1-oxoisoindolin-2-yl) piperidine-2,6-dione (4 mg, yield 7.1%) as a yellow solid. LC-MS (ESI): mass calcd. for C₃₀H₂₉N₅O₃, 507.23; m/z found, 508.23 [M+H]⁺. ¹H NMR (400 MHZ, DMSO-d₆) δ 11.03 (s, 1H), 8.27 (s, 1H), 7.99 (s, 1H), 7.93 (d, J=7.8 Hz, 1H), 7.85 (d, J=8.0 Hz, 1H), 7.50 (s, 1H), 7.44 (s, 1H), 7.40 (d, J=7.3 Hz, 2H), 7.34 (t, J=7.5 Hz, 2H), 7.24 (t, J=7.2 Hz, 1H), 6.66 (s, 1H), 5.20-5.15 (m, 1H), 4.58 (d, J=17.4 Hz, 1H), 4.46 (d, J=17.4 Hz, 1H), 3.73 (d, J=6.4 Hz, 1H), 3.49 (d, J=14.0 Hz, 1H), 3.36 (s, 1H), 2.92 (d, J=12.4 Hz, 1H), 2.62 (d, J=18.2 Hz, 1H), 2.44-2.37 (m, 1H), 2.11 (s, 3H), 2.06-2.02 (m, 1H), 1.38 (d, J=6.7 Hz, 3H).

### Example 18. 3-(1-oxo-5-(7-(((2-phenylpropan-2-yl)amino)methyl) imidazo[1,5-a]pyridin-5-yl) isoindolin-2-yl) piperidine-2,6-dione

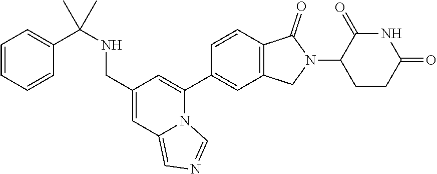 [SMILES: CC(C)(NCc1cc(-c2ccc3c(c2)CN(C2CCC(=O)NC2=O)C3=O)n2cncc2c1)c1ccccc1]

The title compound was prepared in a manner analogous to Example 1 by reductive amination between 2-phenylpropan-2-amine and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₃₀H₂₉N₅O₃, 507.23; m/z found, 508.23 [M+H]⁺. ¹H NMR (400 MHZ, DMSO-d₆) δ 11.04 (s, 1H), 10.02 (s, 2H), 8.97 (s, 1H), 8.09 (s, 1H), 7.98 (d, J=7.8 Hz, 1H), 7.93 (d, J=8.0 Hz, 2H), 7.78 (s, 1H), 7.74 (d, J=7.8 Hz, 2H), 7.48 (t, J=7.6 Hz, 2H), 7.40 (t, J=7.2 Hz, 1H), 7.13 (s, 1H), 5.21-5.17 (m, 1H), 4.59 (d, J=17.6 Hz, 1H), 4.48 (d, J=17.6 Hz, 1H), 3.82 (s, 2H), 2.97-2.86 (m, 1H), 2.63 (d, J=17.4 Hz, 1H), 2.45-2.37 (m, 1H), 2.05 (d, J=5.4 Hz, 1H), 1.84 (s, 6H).

### Example 19. 3-(5-(7-((methyl (2-phenylpropan-2-yl)amino)methyl) imidazo[1,5-a]pyridin-5-yl)-1-oxoisoindolin-2-yl) piperidine-2,6-dione

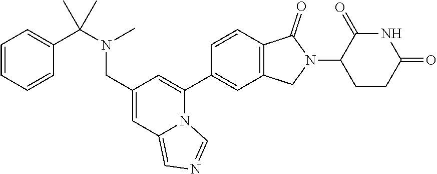 [SMILES: CN(Cc1cc(-c2ccc3c(c2)CN(C2CCC(=O)NC2=O)C3=O)n2cncc2c1)C(C)(C)c1ccccc1]

The title compound was prepared in a manner analogous to Example 17 by reductive amination between 2-phenylpropan-2-amine and 5-chloroimidazo[1,5-a]pyridine-7-carbaldehyde (Intermediate 1), reductive methylation with HCHO and followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₃₁H₃₁N₅O₃, 521.24; m/z found, 522.24 [M+H]⁺. ¹H NMR (400 MHZ, DMSO-d₆) δ 11.03 (s, 1H), 8.26 (s, 1H), 7.98 (s, 1H), 7.93 (d, J=7.8 Hz, 1H), 7.84 (d, J=7.8 Hz, 1H), 7.61 (d, J=7.8 Hz, 2H), 7.52 (s, 1H), 7.44 (s, 1H), 7.33 (t, J=7.8 Hz, 2H), 7.21 (d, J=7.4 Hz, 1H), 6.60 (s, 1H), 5.20-5.15 (m, 1H), 4.58 (d, J=17.6 Hz, 1H), 4.46 (d, J=17.6 Hz, 1H), 3.41 (s, 2H), 2.92 (d, J=12.2 Hz, 1H), 2.62 (d, J=18.3 Hz, 1H), 2.44 (s, 1H), 2.14 (s, 3H), 2.05-2.00 (m, 1H), 1.41 (s, 6H).

### Example 20. 3-(5-(7-(((1-(3-chlorophenyl)ethyl)amino)methyl) imidazo[1,5-a]pyridin-5-yl)-1-oxoisoindolin-2-yl) piperidine-2,6-dione

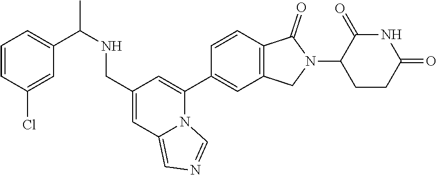 [SMILES: CC(NCc1cc(-c2ccc3c(c2)CN(C2CCC(=O)NC2=O)C3=O)n2cncc2c1)c1cccc(Cl)c1]

The title compound was prepared in a manner analogous to Example 1 by reductive amination between 1-(3-chlorophenyl) ethan-1-amine and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₉H₂₆N₅O₃, 527.17; m/z found, 528.18 [M+H]⁺. ¹H NMR (400 MHz, DMSO-d₆) δ 11.03 (s, 1H), 8.29 (s, 1H), 8.00 (s, 1H), 7.92 (d, J=7.8 Hz, 1H), 7.86 (d, J=7.8 Hz, 1H), 7.44 (s, 3H), 7.34 (d, J=7.2 Hz, 2H), 7.27 (d, J=6.8 Hz, 1H), 6.72 (s, 1H), 5.20-5.16 (m, 1H), 4.58 (d, J=17.4 Hz, 1H), 4.46 (d, J=17.7 Hz, 1H), 3.78-3.74 (m, 1H), 3.56-3.46 (m, 2H), 2.96-2.91 (m, 1H), 2.65-2.62 (m, 1H), 2.47-2.42 (m, 1H), 2.08-2.02 (m, 1H), 1.29 (d, J=6.5 Hz, 3H).

### Example 21. 3-(5-(7-(((2-chlorophenethyl)amino)methyl) imidazo[1,5-a]pyridin-5-yl)-1-oxoisoindolin-2-yl) piperidine-2,6-dione

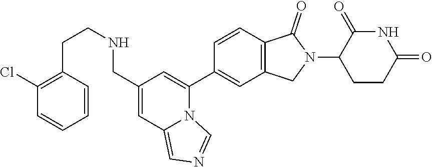 [SMILES: O=C1CCC(N2Cc3cc(-c4cc(CNCCc5ccccc5Cl)cc5cncn45)ccc3C2=O)C(=O)N1]

The title compound was prepared in a manner analogous to Example 1 by reductive amination between 2-(2-chlorophenyl) ethan-1-amine and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₉H₂₆ClN₅O₃, 527.17; m/z found, 528.17 [M+H]⁺. ¹H NMR (400 MHZ, DMSO-d₆) δ 11.03 (s, 1H), 8.30 (s, 1H), 8.00 (s, 1H), 7.93 (d, J=7.8 Hz, 1H), 7.85 (d, J=9.2 Hz, 1H), 7.48 (s, 1H), 7.43 (s, 1H), 7.39-7.34 (m, 2H), 7.26-7.21 (m, 2H), 6.74 (d, J=1.2 Hz, 1H), 5.20-5.16 (m, 1H), 4.57 (d, J=17.6 Hz, 1H), 4.46 (d, J=17.6 Hz, 1H), 3.75 (s, 2H), 2.98-2.93 (m, 1H), 2.91-2.86 (m, 2H), 2.78 (t, J=7.1 Hz, 2H), 2.67-2.63 (m, 1H), 2.46-2.43 (m, 1H), 2.07-2.02 (m, 1H).

### Example 22. 3-(5-(7-(((3-chloro-4-methylphenethyl)amino)methyl) imidazo[1,5-alpyridin-5-yl)-1-oxoisoindolin-2-yl) piperidine-2,6-dione

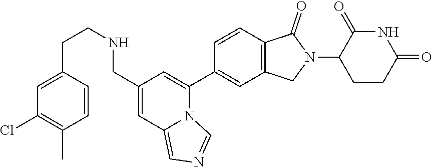 [SMILES: Cc1ccc(CCNCc2cc(-c3ccc4c(c3)CN(C3CCC(=O)NC3=O)C4=O)n3cncc3c2)cc1Cl]

The title compound was prepared in a manner analogous to Example 1 by reductive amination between 2-(3-chloro-4-methylphenyl) ethan-1-amine (Intermediate 20) and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₃₀H₂₈ClN₅O₃, 541.19; m/z found, 542.19 [M+H]⁺. ¹H NMR (400 MHZ, DMSO-d₆) δ 11.04 (s, 1H), 8.30 (d, J=10.2 Hz, 3H), 8.00 (s, 1H), 7.91 (d, J=7.8 Hz, 1H), 7.83 (d, J=7.8 Hz, 1H), 7.43 (d, J=17.2 Hz, 2H), 7.26 (s, 1H), 7.20 (d, J=7.8 Hz, 1H), 7.08 (d, J=6.8 Hz, 1H), 6.71 (s, 1H), 5.20-5.15 (m, 1H), 4.57 (d, J=17.6 Hz, 1H), 4.46 (d, J=17.6 Hz, 1H), 3.70 (s, 2H), 2.97-2.90 (m, 1H), 2.74-2.70 (m, 4H), 2.64-2.60 (m, 1H), 2.46-2.44 (m, 1H), 2.25 (s, 3H), 2.06-2.03 (m, 1H).

### Example 23. 3-(5-(7-(((1-(2-chlorophenyl)ethyl)amino)methyl) imidazo[1,5-a]pyridin-5-yl)-1-oxoisoindolin-2-yl) piperidine-2,6-dione

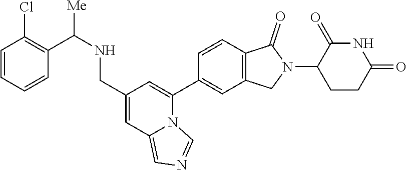 [SMILES: CC(NCc1cc(-c2ccc3c(c2)CN(C2CCC(=O)NC2=O)C3=O)n2cncc2c1)c1ccccc1Cl]

The title compound was prepared in a manner analogous to Example 1 by reductive amination between 1-(2-chlorophenyl) ethan-1-amine and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₉H₂₆N₅O₃, 527.17; m/z found, 528.18 [M+H]⁺. ¹H NMR (400 MHZ, DMSO-d₆) δ 11.03 (s, 1H), 8.31 (s, 1H), 8.01 (s, 1H), 7.93 (d, J=7.8 Hz, 1H), 7.86 (d, J=7.8 Hz, 1H), 7.72 (d, J=7.4 Hz, 1H), 7.46 (s, 2H), 7.38 (t, J=7.8 Hz, 2H), 7.25 (t, J=7.2 Hz, 1H), 6.75 (s, 1H), 5.20-5.16 (m, 1H), 4.58 (d, J=17.6 Hz, 1H), 4.46 (d, J=17.6 Hz, 1H), 4.23 (s, 1H), 3.57-3.52 (m, 2H), 2.94-2.89 (m, 1H), 2.65-2.61 (m, 1H), 2.47-2.43 (m, 1H), 2.08-2.02 (m, 1H), 1.29 (d, J=5.2 Hz, 3H).

### Example 24. 3-(5-(7-(((naphthalen-1-ylmethyl)amino)methyl) imidazo[1,5-a]pyridin-5-yl)-1-oxoisoindolin-2-yl) piperidine-2,6-dione

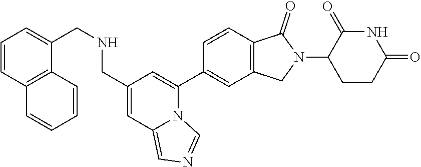 [SMILES: O=C1CCC(N2Cc3cc(-c4cc(CNCc5cccc6ccccc56)cc5cncn45)ccc3C2=O)C(=O)N1]

The title compound was prepared in a manner analogous to Example 1 by reductive amination between naphthalen-1-ylmethanamine and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₃₂H₂₇N₅O₃, 529.21; m/z found, 530.21 [M+H]⁺. ¹H NMR (400 MHZ, DMSO-d₆) δ 11.04 (s, 1H), 8.33 (s, 1H), 8.18 (d, J=5.4 Hz, 1H), 7.99 (s, 1H), 7.93 (d, J=8.0 Hz, 2H), 7.85 (d, J=9.0 Hz, 2H), 7.63-7.56 (m, 2H), 7.55-7.45 (m, 4H), 6.83 (s, 1H), 5.20-5.16 (m, 1H), 4.58 (d, J=17.6 Hz, 1H), 4.46 (d, J=17.6 Hz, 1H), 4.26 (s, 2H), 3.91 (s, 2H), 3.00-2.89 (m, 1H), 2.65-2.61 (m, 1H), 2.48-2.41 (m, 1H), 2.09-2.02 (m, 1H).

### Example 25. 3-(5-(7-((((1-methyl-1H-indol-7-yl)methyl)amino)methyl) imidazo[1,5-alpyridin-5-yl)-1-oxoisoindolin-2-yl) piperidine-2,6-dione

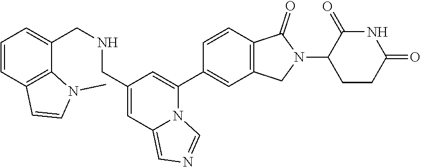 [SMILES: Cn1ccc2cccc(CNCc3cc(-c4ccc5c(c4)CN(C4CCC(=O)NC4=O)C5=O)n4cncc4c3)c21]

The title compound was prepared in a manner analogous to Example 1 by reductive amination between (1-methyl-1H-indol-7-yl) methanamine (Intermediate 21) and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calced for: Chemical Formula: C₃₁H₂₈N₆O₃ 532.22; m/z found, 533.4 [M+H]⁺. ¹HNMR (400 MHZ, DMSO-d₆) δ 11.02 (s, 1H), 8.29 (s, 1H), 8.21 (s, 1H), 7.99 (s, 1H), 7.92 (d, J=7.6 Hz, 1H), 7.85 (d, J=7.8 Hz, 1H), 7.54 (s, 1H), 7.43 (d, J=11.2 Hz, 2H), 7.21 (d, J=3.0 Hz, 1H), 6.98 (d, J=6.8 Hz, 1H), 6.90 (t, J=7.4 Hz, 1H), 6.77 (s, 1H), 6.37 (d, J=3.0 Hz, 1H), 5.20-5.16 (m, 1H), 4.57 (d, J=17.4 Hz, 1H), 4.46 (d, J=17.4 Hz, 1H), 4.11 (s, 3H), 4.08 (s, 2H), 3.79 (s, 2H), 2.99-2.88 (m, 1H), 2.64-2.62 (m, 1H), 2.44 (s, 1H), 2.10-1.99 (m, 1H).

### Example 26. 3-(1-oxo-5-(7-((2-phenylpyrrolidin-1-yl)methyl) imidazo[1,5-a]pyridin-5-yl) isoindolin-2-yl) piperidine-2,6-dione

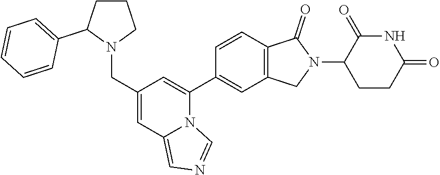 [SMILES: O=C1CCC(N2Cc3cc(-c4cc(CN5CCCC5c5ccccc5)cc5cncn45)ccc3C2=O)C(=O)N1]

The title compound was prepared in a manner analogous to Example 1 by reductive amination between 2-phenylpyrrolidine and 5-chloroimidazo[1,5-a]pyridine-7-carbaldehyde (Intermediate 1) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₁H₂₉N₅O₃, 519.23; m/z found, 520.23 [M+H]⁺. ¹H NMR (400 MHZ, DMSO-d₆) δ 11.02 (s, 1H), 8.24 (d, J=11.4 Hz, 1H), 7.97-7.88 (m, 2H), 7.81 (d, J=7.8 Hz, 1H), 7.47 (s, 1H), 7.42 (d, J=7.2 Hz, 3H), 7.31 (t, J=7.6 Hz, 2H), 7.20 (t, J=7.2 Hz, 1H), 6.54 (s, 1H), 5.18-5.14 (m, 1H), 4.54 (d, J=17.6 Hz, 1H), 4.47 (d, J=17.6 Hz, 1H), 3.63 (d, J=14.0 Hz, 2H), 3.17-3.12 (m, 2H), 2.94-2.91 (m, 1H), 2.65-2.62 (m, 1H), 2.45-2.42 (m, 1H), 2.28 (d, J=8.8 Hz, 1H), 2.18 (d, J=8.8 Hz, 1H), 2.08-2.01 (m, 1H), 1.88-1.79 (m, 2H), 1.67-1.61 (m, 1H).

### Example 27. 3-(4-fluoro-1-oxo-5-(7-(pyrrolidin-1-ylmethyl) imidazo[1,5-a]pyridin-5-yl) isoindolin-2-yl) piperidine-2,6-dione

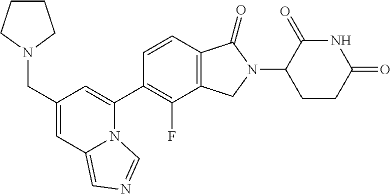 [SMILES: O=C1CCC(N2Cc3c(ccc(-c4cc(CN5CCCC5)cc5cncn45)c3F)C2=O)C(=O)N1]

The title compound was prepared in a manner analogous to Example 1 by reductive amination between pyrrolidine and 5-chloroimidazo[1,5-a]pyridine-7-carbaldehyde (Intermediate 1) followed by Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14).

LC-MS (ESI): mass calcd. for C₂₅H₂₄FN₅O₃, 461.19; m/z found, 462.19 [M+H]⁺. ¹H NMR (400 MHZ, DMSO-d₆) δ 11.05 (s, 1H), 8.13 (d, J=3.4 Hz, 1H), 7.88-7.83 (m, 1H), 7.79 (d, J=7.6 Hz, 1H), 7.64 (s, 1H), 7.50 (s, 1H), 6.83 (s, 1H), 5.21-5.17 (m, 1H), 4.68 (d, J=17.4 Hz, 1H), 4.54 (d, J=17.4 Hz, 1H), 3.76 (s, 2H), 3.00-2.88 (m, 1H), 2.80-2.53 (m, 5H), 2.45 (s, 1H), 2.08-1.99 (m, 1H), 1.77 (s, 4H).

### Example 28. 3-(7-fluoro-1-oxo-5-(7-(pyrrolidin-1-ylmethyl) imidazo[1,5-a]pyridin-5-yl) isoindolin-2-yl) piperidine-2,6-dione

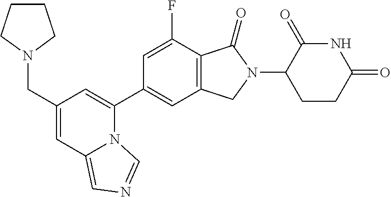 [SMILES: O=C1CCC(N2Cc3cc(-c4cc(CN5CCCC5)cc5cncn45)cc(F)c3C2=O)C(=O)N1]

The title compound was prepared in a manner analogous to Example 1 by reductive amination between pyrrolidine and 5-chloroimidazo[1,5-a]pyridine-7-carbaldehyde (Intermediate 1) followed by Suzuki coupling with 3-(7-fluoro-1-oxo-5-(4,4,5,5-tetramethyl-1,3,2-dioxaborolan-2-yl) isoindolin-2-yl) piperidine-2,6-dione (Intermediate 16).

LC-MS (ESI): mass calcd. for C₂₅H₂₄FN₅O₃, 461.19; m/z found, 462.4 [M+H]⁺. ¹H NMR (400 MHZ, DMSO-d₆) δ 11.05 (s, 1H), 9.98 (s, 1H), 8.66 (s, 1H), 7.92 (s, 1H), 7.86 (s, 1H), 7.75 (s, 1H), 7.73 (s, 1H), 6.99 (s, 1H), 5.17-5.12 (m, 1H), 4.54 (d, J=18.0 Hz, 1H), 4.46 (d, J=18.0 Hz, 1H), 4.37 (d, J=5.0 Hz, 2H), 3.50-3.44 (m, 2H), 3.20-3.10 (m, 2H), 3.00-2.86 (m, 1H), 2.63-2.59 (m, 1H), 2.46-2.45 (m, 1H), 2.06-2.01 (m, 3H), 1.90-1.88 (m, 2H).

### Example 29. 3-(6-fluoro-1-oxo-5-(7-(pyrrolidin-1-ylmethyl) imidazo[1,5-a]pyridin-5-yl) isoindolin-2-yl) piperidine-2,6-dione

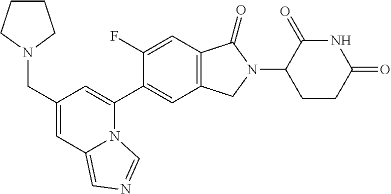 [SMILES: O=C1CCC(N2Cc3cc(-c4cc(CN5CCCC5)cc5cncn45)c(F)cc3C2=O)C(=O)N1]

The title compound was prepared in a manner analogous to Example 1 by reductive amination between pyrrolidine and 5-chloroimidazo[1,5-a]pyridine-7-carbaldehyde (Intermediate 1) followed by Suzuki coupling with 3-(6-fluoro-1-oxo-5-(4,4,5,5-tetramethyl-1,3,2-dioxaborolan-2-yl) isoindolin-2-yl) piperidine-2,6-dione (Intermediate 15).

LC-MS (ESI): mass calcd. for C₂₅H₂₄FN₅O₃, 461.19; m/z found, 462.4 [M+H]⁺. ¹H NMR (400 MHZ, DMSO-d₆) δ 11.04 (s, 1H), 8.21 (s, 1H), 8.01 (d, J=2.6 Hz, 1H), 7.97 (d, J=6.0 Hz, 1H), 7.78 (d, J=8.8 Hz, 1H), 7.56 (s, 1H), 7.45 (s, 1H), 6.75 (s, 1H), 5.20-5.16 (m, 1H), 4.55 (d, J=17.6 Hz, 1H), 4.43 (d, J=17.6 Hz, 1H), 3.59 (s, 2H), 3.29-3.25 (m, 4H), 2.98-2.89 (m, 1H), 2.64-2.59 (m, 1H), 2.47-2.39 (m, 1H), 2.06-2.00 (m, 1H), 1.73-1.72 (m, 4H).

### Example 30. 3-(6-methoxy-1-oxo-5-(7-(pyrrolidin-1-ylmethyl) imidazo[1,5-a]pyridin-5-yl) isoindolin-2-yl) piperidine-2,6-dione

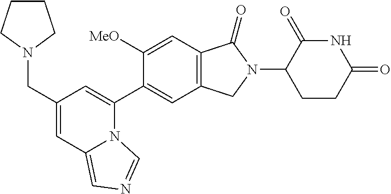 [SMILES: COc1cc2c(cc1-c1cc(CN3CCCC3)cc3cncn13)CN(C1CCC(=O)NC1=O)C2=O]

The title compound was prepared in a manner analogous to Example 1 by reductive amination between pyrrolidine and 5-chloroimidazo[1,5-a]pyridine-7-carbaldehyde (Intermediate 1) followed by Suzuki coupling with 3-(6-methoxy-1-oxo-5-(4,4,5,5-tetramethyl-1,3,2-dioxaborolan-2-yl) isoindolin-2-yl) piperidine-2,6-dione (Intermediate 18).

LC-MS (ESI): mass calcd. for C₂₆H₂₇N₅O₄, 473.53; m/z found, 474.4 [M+H]⁺. ¹H NMR (400 MHZ, DMSO-d₆) δ 11.03 (s, 1H), 10.05 (s, 1H), 8.10 (s, 1H), 7.87 (s, 1H), 7.75 (s, 1H), 7.72 (s, 1H), 7.55 (s, 1H), 6.91 (s, 1H), 5.21-5.16 (m, 1H), 4.48 (d, J=17.0 Hz, 1H), 4.43-4.32 (m, 3H), 3.86 (s, 3H), 3.17-3.14 (m, 4H), 3.00-2.88 (m, 1H), 2.67-2.56 (m, 1H), 2.46-2.39 (m, 1H), 2.07-1.86 (m, 5H). ¹⁹F NMR (377 MHz, DMSO-d₆) δ-73.96.

### Example 31. 3-(4-methoxy-1-oxo-5-(7-(pyrrolidin-1-ylmethyl) imidazo[1,5-a]pyridin-5-yl) isoindolin-2-yl) piperidine-2,6-dione

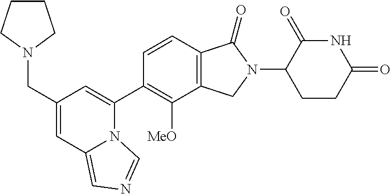 [SMILES: COc1c(-c2cc(CN3CCCC3)cc3cncn23)ccc2c1CN(C1CCC(=O)NC1=O)C2=O]

The title compound was prepared in a manner analogous to Example 1 by reductive amination between pyrrolidine and 5-chloroimidazo[1,5-a]pyridine-7-carbaldehyde (Intermediate 1) followed by Suzuki coupling with 3-(4-methoxy-1-oxo-5-(4,4,5,5-tetramethyl-1,3,2-dioxaborolan-2-yl) isoindolin-2-yl) piperidine-2,6-dione (Intermediate 17).

LC-MS (ESI): mass calced for: Chemical Formula: C₂₆H₂₇N₅O₄ 473.21; m/z found, 474.2 [M+H]⁺. ¹H NMR (400 MHZ, DMSO-d₆) δ 11.06 (s, 1H), 10.33 (s, 1H), 8.67-8.63 (m, 1H), 7.94 (s, 2H), 7.66 (d, J=7.6 Hz, 1H), 7.59 (d, J=7.6 Hz, 1H), 7.04 (s, 1H), 5.22-5.18 (m, 1H), 4.90 (d, J=17.2 Hz, 1H), 4.76 (d, J=17.2 Hz, 1H), 4.42 (s, 2H), 3.91 (s, 3H), 3.49 (s, 2H), 3.16 (s, 2H), 3.02-2.88 (m, 1H), 2.64 (d, J=17.6 Hz, 1H), 2.59-2.52 (m, 1H), 2.09-2.03 (m, 3H), 1.90 (d, J=5.2 Hz, 2H).

### Example 32. 3-(7-methoxy-1-oxo-5-(7-(pyrrolidin-1-ylmethyl) imidazo[1,5-a]pyridin-5-yl) isoindolin-2-yl) piperidine-2,6-dione

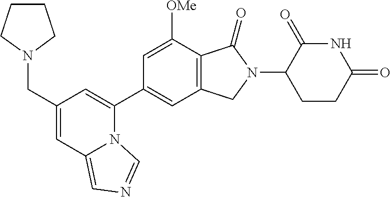 [SMILES: COc1cc(-c2cc(CN3CCCC3)cc3cncn23)cc2c1C(=O)N(C1CCC(=O)NC1=O)C2]

The title compound was prepared in a manner analogous to Example 1 by reductive amination between pyrrolidine and 5-chloroimidazo[1,5-a]pyridine-7-carbaldehyde (Intermediate 1) followed by Suzuki coupling with 3-(7-methoxy-1-oxo-5-(4,4,5,5-tetramethyl-1,3,2-dioxaborolan-2-yl) isoindolin-2-yl) piperidine-2,6-dione (Intermediate 19).

LC-MS (ESI): mass calcd. for C₂₆H₂₇N₅O₄, 473.21; m/z found, 474.4 [M+H]⁺. ¹HNMR (400 MHZ, DMSO-d₆) 11.00 (s, 1H), 10.36 (s, 1H), 8.80 (s, 1H), 7.88 (s, 1H), 7.83 (s, 1H), 7.54 (s, 1H), 7.45 (s, 1H), 7.06 (s, 1H), 5.12-5.07 (m, 1H), 4.47 (d, J=16.8 Hz, 1H), 4.39-4.33 (m, 3H), 3.96 (s, 3H), 3.49 (s, 2H), 3.16 (s, 2H), 3.01-2.89 (m, 1H), 2.63-2.58 (m, 1H), 2.43-2.40 (m, 1H), 2.07-2.00 (m, 3H), 1.99-1.88 (m, 2H).

### Example 33. 3-(1-oxo-5-(6-(piperidin-1-ylmethyl) imidazo[1,2-a]pyridin-8-yl) isoindolin-2-yl) piperidine-2,6-dione

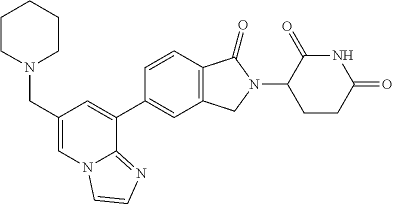 [SMILES: O=C1CCC(N2Cc3cc(-c4cc(CN5CCCCC5)cn5ccnc45)ccc3C2=O)C(=O)N1]

The title compound was prepared in a manner analogous to Example 1 by reductive amination between piperidine and 8-bromoimidazo[1,2-a]pyridine-6-carbaldehyde (Intermediate 2) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₆H₂₇N₅O₃, 457.21; m/z found, 458.21 [M+H]⁺. ¹HNMR (400 MHZ, DMSO-d₆) δ 11.02 (s, 1H), 8.47 (s, 3H), 8.31 (d, J=7.8 Hz, 1H), 8.12 (d, J=14.2 Hz, 1H), 7.87 (d, J=8.0 Hz, 1H), 7.68 (s, 1H), 5.18-5.13 (m, 1H), 4.57 (d, J=17.4 Hz, 1H), 4.43 (d, J=17.4 Hz, 1H), 3.35 (s, 2H), 3.01-2.97 (m, 2H), 2.96-2.87 (m, 1H), 2.69-2.55 (m, 2H), 2.43 (dd, J=13.2, 8.6 Hz, 2H), 2.09-2.00 (m, 1H), 1.70-1.61 (m, 4H), 1.55 (d, J=5.0 Hz, 2H).

### Example 34. 3-(5-(6-(azetidin-1-ylmethyl) imidazo[1,2-a]pyridin-8-yl)-1-oxoisoindolin-2-yl) piperidine-2,6-dione

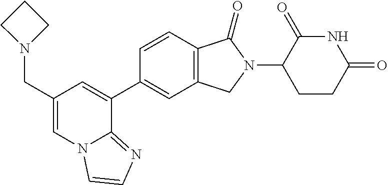 [SMILES: O=C1CCC(N2Cc3cc(-c4cc(CN5CCC5)cn5ccnc45)ccc3C2=O)C(=O)N1]

The title compound was prepared in a manner analogous to Example 1 by reductive amination between azetidine and 8-bromoimidazo[1,2-a]pyridine-6-carbaldehyde (Intermediate 2) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₄H₂₃N₅O₃, 429.18; m/z found, 430.19 [M+H]⁺. ¹H NMR (400 MHZ, DMSO-d₆) δ 11.02 (s, 1H), 8.51 (s, 1H), 8.41 (s, 1H), 8.29 (d, J=7.8 Hz, 1H), 8.04 (d, J=1.0 Hz, 1H), 7.84 (d, J=8.0 Hz, 1H), 7.62 (d, J=1.0 Hz, 1H), 7.49 (d, J=1.0 Hz, 1H), 5.18-5.14 (m, 1H), 4.56 (d, J=17.4 Hz, 1H), 4.43 (d, J=17.4 Hz, 1H), 3.60 (s, 2H), 3.20 (t, J=7.0 Hz, 4H), 3.01-2.87 (m, 1H), 2.63 (d, J=16.6 Hz, 1H), 2.46-2.42 (m, 1H), 2.08-1.96 (m, 3H).

### Example 35. 3-(5-(6-((4,4-difluoropiperidin-1-yl)methyl) imidazo[1,2-a]pyridin-8-yl)-1-oxoisoindolin-2-yl) piperidine-2,6-dione

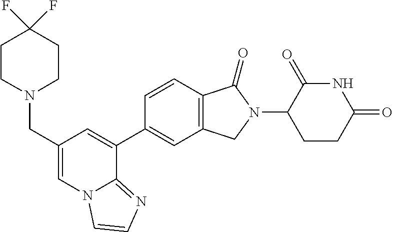 [SMILES: O=C1CCC(N2Cc3cc(-c4cc(CN5CCC(F)(F)CC5)cn5ccnc45)ccc3C2=O)C(=O)N1]

The title compound was prepared in a manner analogous to Example 1 by reductive amination between 4,4-difluoropiperidine and 8-bromoimidazo[1,2-a]pyridine-6-carbaldehyde (Intermediate 2) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. For C₂₆H₂₅F₂N₅O₃, 493.19; m/z found, 494.20 [M+H]⁺. ¹HNMR (400 MHZ, DMSO-d₆) δ 11.02 (s, 1H), 8.56 (s, 1H), 8.42 (s, 1H), 8.29 (d, J=8.0 Hz, 1H), 8.04 (d, J=1.0 Hz, 1H), 7.85 (d, J=8.0 Hz, 1H), 7.64 (d, J=1.2 Hz, 1H), 7.56 (d, J=1.2 Hz, 1H), 5.19-5.14 (m, 1H), 4.57 (d, J=17.4 Hz, 1H), 4.43 (d, J=17.4 Hz, 1H), 3.64 (s, 2H), 3.01-2.87 (m, 1H), 2.68-2.54 (m, 5H), 2.45-2.38 (m, 1H), 2.07-1.94 (m, 5H).

### Example 36. 3-(5-(6-((4,4-dimethylpiperidin-1-yl)methyl) imidazo[1,2-a]pyridin-8-yl)-1-oxoisoindolin-2-yl) piperidine-2,6-dione

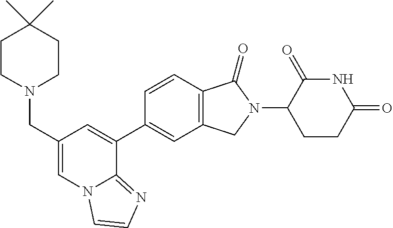 [SMILES: CC1(C)CCN(Cc2cc(-c3ccc4c(c3)CN(C3CCC(=O)NC3=O)C4=O)c3nccn3c2)CC1]

The title compound was prepared in a manner analogous to Example 1 by reductive amination between 4,4-dimethylpiperidine and 8-bromoimidazo[1,2-a]pyridine-6-carbaldehyde (Intermediate 2) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₈H₃₁N₅O₃, 485.24; m/z found, 486.24 [M+H]⁺. ¹H NMR (400 MHZ, DMSO-d₆) δ 11.02 (s, 1H), 8.55 (s, 1H), 8.42 (s, 1H), 8.28 (d, J=7.8 Hz, 1H), 8.06 (s, 1H), 7.85 (d, J=8.0 Hz, 1H), 7.64 (s, 1H), 7.54 (s, 1H), 5.18-5.14 (m, 1H), 4.57 (d, J=17.4 Hz, 1H), 4.43 (d, J=17.4 Hz, 1H), 3.55 (s, 2H), 2.94 (s, 1H), 2.66 (d, J=8.8 Hz, 3H), 2.43-2.39 (m, 3H), 2.05 (s, 1H), 1.34 (d, J=6.0 Hz, 4H), 1.23 (s, 6H).

### Example 37. 3-(1-oxo-5-(6-((4-(trifluoromethyl) piperidin-1-yl)methyl) imidazo[1,2-a]pyridin-8-yl) isoindolin-2-yl) piperidine-2,6-dione

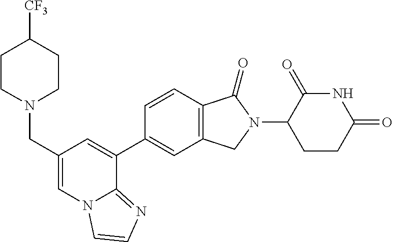 [SMILES: O=C1CCC(N2Cc3cc(-c4cc(CN5CCC(C(F)(F)F)CC5)cn5ccnc45)ccc3C2=O)C(=O)N1]

The title compound was prepared in a manner analogous to Example 1 by reductive amination between 4-trifluoromethylpiperidine and 8-bromoimidazo[1,2-a]pyridine-6-carbaldehyde (Intermediate 2) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₇H₂₆F₃N₅O₃, 525.10; m/z found, 526 [M+H]⁺. ¹H NMR (400 MHZ, DMSO-d₆) δ 11.02 (s, 1H), 8.55 (s, 1H), 8.41 (s, 1H), 8.28 (d, J=8.2 Hz, 1H), 8.05 (d, J=1.0 Hz, 1H), .85 (d, J=8.0 Hz, 1H), 7.64 (d, J=1.0 Hz, 1H), 7.53 (d, J=1.2 Hz, 1H), 5.19-5.14 (m, 1H), 4.57 (d, J=17.4 Hz, 1H), 4.43 (d, J=17.4 Hz, 1H), 3.58 (s, 2H), 3.02-2.91 (m, 3H), 2.63 (d, J=17.4 Hz, 1H), 2.44 (dd, J=13.2, 4.4 Hz, 1H), 2.29 (d, J=8.4 Hz, 1H), 2.07-2.02 (m, 3H), 1.80-1.77 (m, 2H), 1.51-1.47 (m, 2H).

### Example 38. 3-(5-(6-(((2-chlorobenzyl)amino)methyl) imidazo[1,2-a]pyridin-8-yl)-1-oxoisoindolin-2-yl) piperidine-2,6-dione

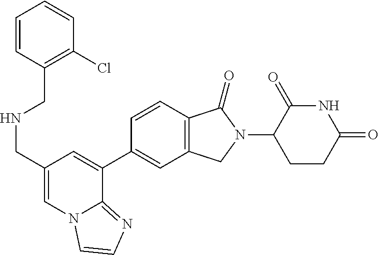 [SMILES: O=C1CCC(N2Cc3cc(-c4cc(CNCc5ccccc5Cl)cn5ccnc45)ccc3C2=O)C(=O)N1]

The title compound was prepared in a manner analogous to Example 1 by reductive amination between (2-chlorophenyl) methanamine and 8-bromoimidazo[1,2-a]pyridine-6-carbaldehyde (Intermediate 2) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₈H₂₄ClN₅O₃, 513.16; m/z found, 514.16 [M+H]⁺. ¹H NMR (400 MHZ, DMSO-d₆) δ 11.01 (s, 1H), 8.56 (s, 1H), 8.44 (s, 1H), 8.31 (d, J=8.2 Hz, 1H), 8.04 (d, J=1.0 Hz, 1H), 7.85 (d, J=8.0 Hz, 1H), 7.66 (d, J=1.2 Hz, 1H), 7.63-7.60 (m, 2H), 7.42-7.39 (m, 1H), 7.34 (dd, J=7.4, 6.2 Hz, 1H), 7.27-7.25 (m,, 1H), 5.19-5.14 (m, 1H), 4.56 (d, J=17.4 Hz, 1H), 4.43 (d, J=17.4 Hz, 1H), 3.83 (s, 4H), 2.98-2.90 (m, 1H), 2.64-2.60 (m, 1H), 2.47-2.43 (m, 1H), 2.06-2.00 (m, 1H).

### Example 39. 3-(5-(6-(((2,6-difluorophenethyl)amino)methyl) imidazo[1,2-a]pyridin-8-yl)-1-oxoisoindolin-2-yl) piperidine-2,6-dione

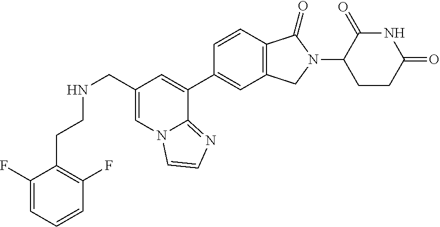 [SMILES: O=C1CCC(N2Cc3cc(-c4cc(CNCCc5c(F)cccc5F)cn5ccnc45)ccc3C2=O)C(=O)N1]

The title compound was prepared in a manner analogous to Example 1 by reductive amination between 2-(2,6-difluorophenyl) ethan-1-amine and 8-bromoimidazo[1,2-a]pyridine-6-carbaldehyde (Intermediate 2) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₉H₂₅F₂N₅O₃, 529.19; m/z found, 530.20 [M+H]⁺. ¹H NMR (400 MHZ, DMSO-d₆) δ 11.03 (s, 1H), 8.49 (s, 1H), 8.41 (s, 1H), 8.27 (d, J=8.2 Hz, 1H), 8.01 (s, 1H), 7.86 (d, J=8.0 Hz, 1H), 7.62 (d, J=10.0 Hz, 2H), 7.33-7.25 (m, 1H), 7.04 (t, J=7.8 Hz, 2H), 5.19-5.15 (m, 1H), 4.57 (d, J=17.4 Hz, 1H), 4.43 (d, J=17.4 Hz, 1H), 3.82 (s, 2H), 2.96-2.90 (m, 1H), 2.83-2.75 (m, 4H), 2.67-2.65 (m, 1H), 2.45-2.42 (m, 1H), 2.09-2.02 (m, 1H).

### Example 40. 3-(5-(6-(((2,4-dichlorophenethyl)amino) methyl) imidazo[1,2-a]pyridin-8-yl)-1-oxoisoindolin-2-yl) piperidine-2,6-dione

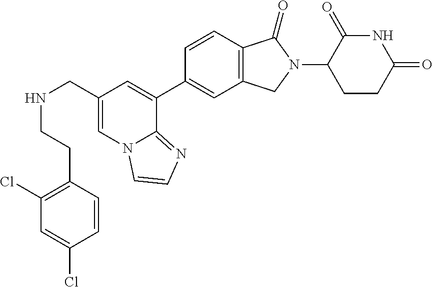 [SMILES: O=C1CCC(N2Cc3cc(-c4cc(CNCCc5ccc(Cl)cc5Cl)cn5ccnc45)ccc3C2=O)C(=O)N1]

The title compound was prepared in a manner analogous to Example 1 by reductive amination between 2-(2,4-dichlorophenyl) ethan-1-amine and 8-bromoimidazo[1,2-a]pyridine-6-carbaldehyde (Intermediate 2) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₉H₂₅C₁₂N₅O₃, 561.13; m/z found, 562.14 [M+H]⁺. ¹H NMR (400 MHZ, DMSO-d₆) δ 11.02 (s, 1H), 8.49 (s, 1H), 8.42 (s, 1H), 8.27 (d, J=8.2 Hz, 1H), 8.01 (d, J=1.2 Hz, 1H), 7.85 (d, J=8.2 Hz, 1H), 7.61 (dd, J=13.2, 1.2 Hz, 2H), 7.53 (d, J=2.0 Hz, 1H), 7.40 (d, J=8.2 Hz, 1H), 7.33 (dd, J=8.2, 2.1 Hz, 1H), 5.19-5.14 (m, 1H), 4.57 (d, J=17.2 Hz, 1H), 4.43 (d, J=17.2 Hz, 1H), 3.81 (s, 2H), 3.00-2.84 (m, 4H), 2.79-2.76 (m, 1H), 2.66-2.58 (m, 1H), 2.45-2.40 (m, 1H), 2.09-2.01 (m, 1H).

### Example 41. 3-(5-(6-(((2,6-dichlorophenethyl)amino) methyl) imidazo[1,2-a]pyridin-8-yl)-1-oxoisoindolin-2-yl) piperidine-2,6-dione

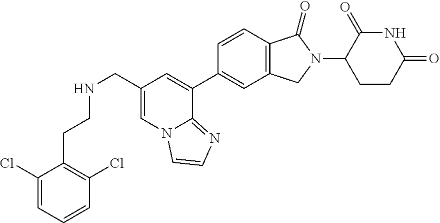 [SMILES: O=C1CCC(N2Cc3cc(-c4cc(CNCCc5c(Cl)cccc5Cl)cn5ccnc45)ccc3C2=O)C(=O)N1]

The title compound was prepared in a manner analogous to Example 1 by reductive amination between 2-(2,6-dichlorophenyl) ethan-1-amine and 8-bromoimidazo[1,2-a]pyridine-6-carbaldehyde (Intermediate 2) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₉H₂₅Cl₂N₅O₃, 561.13; m/z found, 562.14 [M+H]⁺. ¹H NMR (400 Mhz, DMSO-d₆): δ11.02 (s, 1H), 8.52 (s, 1H), 8.43 (s, 1H), 8.29 (d, J=7.8 Hz, 1H), 8.01 (d, J=1.2 Hz, 1H), 7.85 (d, J=8.0 Hz, 1H), 7.63 (dd, J=5.2, 1.2 Hz, 2H), 7.41 (d, J=8.0 Hz, 2H), 7.26-7.20 (m, 1H), 5.19-5.14 (m, 1H), 4.57 (d, J=17.4 Hz, 1H), 4.43 (d, J=17.4 Hz, 1H), 3.84 (s, 2H), 3.11-3.05 (m, 2H), 2.96-2.90 (m, 1H), 2.75-2.70 (m, 2H), 2.65-2.58 (m, 1H), 2.44-2.43 (m, 1H), 2.09-2.03 (m, 1H).

### Example 42. 3-(5-(6-(((2-methylphenethyl)amino)methyl) imidazo[1,2-a]pyridin-8-yl)-1-oxoisoindolin-2-yl) piperidine-2,6-dione

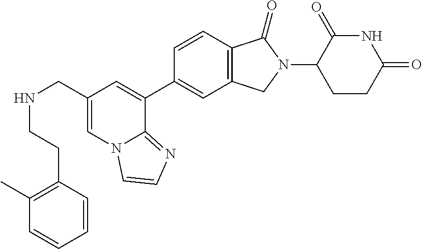 [SMILES: Cc1ccccc1CCNCc1cc(-c2ccc3c(c2)CN(C2CCC(=O)NC2=O)C3=O)c2nccn2c1]

The title compound was prepared in a manner analogous to Example 1 by reductive amination between 2-(2-methylphenyl) ethan-1-amine and 8-bromoimidazo[1,2-a]pyridine-6-carbaldehyde (Intermediate 2) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₃₀H₂₉N₅O₃, 507.23; m/z found, 508.23 [M+H]⁺. ¹H NMR (400 MHZ, DMSO-d₆) δ 11.03 (s, 1H), 8.55 (s, 1H), 8.44 (s, 1H), 8.29 (d, J=8.2 Hz, 1H), 8.04 (s, 1H), 7.86 (d, J=8.0 Hz, 1H), 7.68-7.61 (m, 2H), 7.16-7.08 (m, 4H), 5.19-5.14 (m, 1H), 4.57 (d, J=17.4 Hz, 1H), 4.43 (d, J=17.4 Hz, 1H), 3.91 (s, 2H), 2.95-2.89 (m, 1H), 2.80 (s, 4H), 2.67-2.64 (m, 1H), 2.44-2.42 (m, 1H), 2.26 (s, 3H), 2.08-2.02 (m, 1H).

### Example 43. 3-(1-oxo-5-(6-(((2-(trifluoromethyl) phenethyl)amino)methyl) imidazo[1,2-alpyridin-8-yl) isoindolin-2-yl) piperidine-2,6-dione

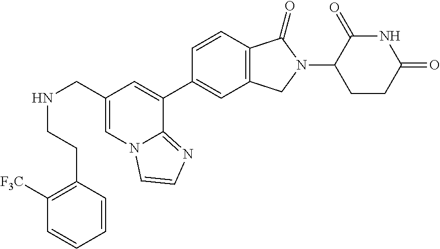 [SMILES: O=C1CCC(N2Cc3cc(-c4cc(CNCCc5ccccc5C(F)(F)F)cn5ccnc45)ccc3C2=O)C(=O)N1]

The title compound was prepared in a manner analogous to Example 1 by reductive amination between 2-(2-trifluoromethylphenyl) ethan-1-amine and 8-bromoimidazo[1,2-alpyridine-6-carbaldehyde (Intermediate 2) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₃₀H₂₆F₃N₅O₃, 561.20; m/z found, 562.20 [M+H]⁺. ¹H NMR (400 Mhz, DMSO-d₆): δ11.02 (s, 1H), 8.54 (s, 1H), 8.43 (s, 1H), 8.29 (d, J=8.0 Hz, 1H), 8.04 (s, 1H), 7.86 (d, J=8.2 Hz, 1H), 7.65 (dd, J=10.4, 4.7 Hz, 3H), 7.59 (t, J=7.4 Hz, 1H), 7.51 (d, J=7.4 Hz, 1H), 7.41 (t, J=7.8 Hz, 1H), 5.19-5.14 (m, 1H), 4.57 (d, J=17.4 Hz, 1H), 4.43 (d, J=17.4 Hz, 1H), 3.88 (s, 2H), 2.98-2.96 (m, 3H), 2.92-2.83 (m, 2H), 2.67-2.64 (m, 1H), 2.46-2.40 (m, 1H), 2.09-2.02 (m, 1H).

### Example 44. 3-(5-(6-(((2-chlorophenethyl)amino)methyl) imidazo[1,2-a]pyridin-8-yl)-1-oxoisoindolin-2-yl) piperidine-2,6-dione

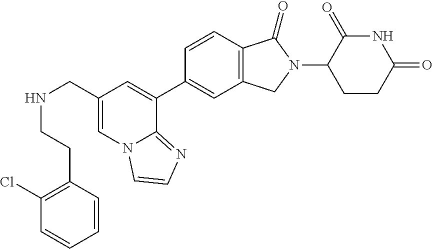 [SMILES: O=C1CCC(N2Cc3cc(-c4cc(CNCCc5ccccc5Cl)cn5ccnc45)ccc3C2=O)C(=O)N1]

The title compound was prepared in a manner analogous to Example 1 by reductive amination between 2-(2-chlorophenyl) ethan-1-amine and 8-bromoimidazo[1,2-a]pyridine-6-carbaldehyde (Intermediate 2) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₉H₂₆ClN₅O₃, 527.17; m/z found, 528.17 [M+H]⁺. ¹H NMR (400 MHZ, DMSO-d₆) δ 11.02 (s, 1H), 8.50 (s, 1H), 8.41 (s, 1H), 8.27 (d, J=8.0 Hz, 1H), 8.01 (d, J=1.2 Hz, 1H), 7.86 (d, J=8.0 Hz, 1H), 7.62 (dd, J=7.2, 1.0 Hz, 2H), 7.39-7.35 (m, 2H), 7.26-7.21 (m, 2H), 5.18-5.14 (m, 1H), 4.57 (d, J=17.4 Hz, 1H), 4.44 (d, J=17.4 Hz, 1H), 3.83 (s, 2H), 2.89 (d, J=7.4 Hz, 2H), 2.81-2.77 (m, 2H), 2.63 (d, J=19.2 Hz, 1H), 2.44 (d, J=4.4 Hz, 2H), 2.08-2.03 (m, 1H).

### Example 45. 3-(1-oxo-5-(6-(((1-phenylethyl)amino)methyl) imidazo[1,2-a]pyridin-8-yl) isoindolin-2-yl) piperidine-2,6-dione

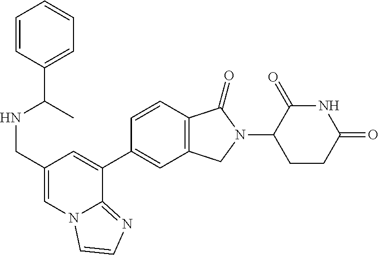 [SMILES: CC(NCc1cc(-c2ccc3c(c2)CN(C2CCC(=O)NC2=O)C3=O)c2nccn2c1)c1ccccc1]

The title compound was prepared in a manner analogous to Example 1 by reductive amination between 1-phenylethan-1-amine and 8-bromoimidazo[1,2-a]pyridine-6-carbaldehyde (Intermediate 2) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₉H₂₇N₅O₃, 493.21; m/z found, 494.21 [M+H]⁺. ¹H NMR (400 MHz, DMSO-d₆) δ 11.01 (s, 1H), 8.44 (d, J=15.2 Hz, 2H), 8.29 (d, J=9.1 Hz, 1H), 8.03 (s, 1H), 7.85 (d, J=8.0 Hz, 1H), 7.64-7.55 (m, 2H), 7.40 (d, J=7.2 Hz, 2H), 7.34 (t, J=7.6 Hz, 2H), 7.23 (t, J=7.2 Hz, 1H), 5.18-5.13 (m, 1H), 4.56 (d, J=17.4 Hz, 1H), 4.42 (d, J=17.4 Hz, 1H), 3.79 (s, 1H), 3.61 (s, 2H), 2.99-2.89 (m, 1H), 2.62 (d, J=18.0 Hz, 1H), 2.44-2.34 (m, 1H), 2.05 (dd, J=10.2, 5.0 Hz, 1H), 1.23 (d, J=3.6 Hz, 3H).

### Example 46. 3-(1-oxo-5-(6-(((2-phenylpropan-2-yl)amino)methyl) imidazo[1,2-a]pyridin-8-yl) isoindolin-2-yl) piperidine-2,6-dione

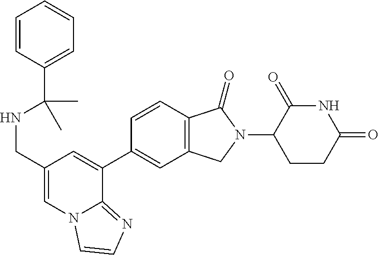 [SMILES: CC(C)(NCc1cc(-c2ccc3c(c2)CN(C2CCC(=O)NC2=O)C3=O)c2nccn2c1)c1ccccc1]

The title compound was prepared in a manner analogous to Example 1 by reductive amination between 2-phenylpropan-2-amine and 8-bromoimidazo[1,2-a]pyridine-6-carbaldehyde (Intermediate 2) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₃₀H₂₉N₅O₃, 507.23; m/z found, 508.23 [M+H]⁺. ¹H NMR (400 MHZ, DMSO-d₆) δ 11.01 (s, 1H), 8.54 (s, 1H), 8.42 (s, 1H), 8.29 (d, J=7.8 Hz, 1H), 8.06 (s, 1H), 7.85 (d, J=8.0 Hz, 1H), 7.62 (s, 2H), 7.54 (d, J=7.4 Hz, 1H), 7.48-7.31 (m, 3H), 7.23 (s, 1H), 5.18-5.13 (m, 1H), 4.56 (d, J=17.4 Hz, 1H), 4.43 (d, J=17.4 Hz, 1H), 3.49 (s, 2H), 3.01-2.88 (m, 1H), 2.62 (d, J=17.4 Hz, 1H), 2.46-2.38 (m, 1H), 2.10-1.99 (m, 1H), 1.50 (s, 6H).

### Example 47. 3-(1-oxo-5-(6-((4-phenylpiperidin-1-yl)methyl) imidazo[1,2-a]pyridin-8-yl) isoindolin-2-yl) piperidine-2,6-dione

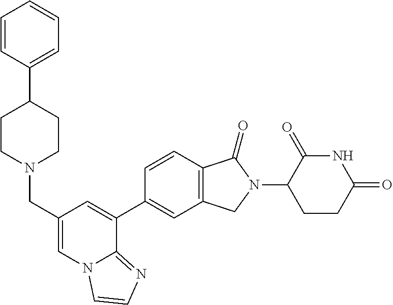 [SMILES: O=C1CCC(N2Cc3cc(-c4cc(CN5CCC(c6ccccc6)CC5)cn5ccnc45)ccc3C2=O)C(=O)N1]

The title compound was prepared in a manner analogous to Example 1 by reductive amination between 4-phenylpiperidine and 8-bromoimidazo[1,2-a]pyridine-6-carbaldehyde (Intermediate 2) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₃₂H₃₁N₅O₃, 533.24; m/z found, 534 [M+H]⁺. ¹H NMR (400 MHZ, DMSO-d₆) δ 11.02 (s, 1H), 8.57 (s, 1H), 8.42 (s, 1H), 8.30 (d, J=8.0 Hz, 1H), 8.05 (d, J=1.2 Hz, 1H), 7.86 (d, J=8.2 Hz, 1H), 7.64 (d, J=1.2 Hz, 1H), 7.58 (s, 1H), 7.28-7.23 (m, 4H), 7.17 (t, J=6.8 Hz, 1H), 5.19-5.14 (m, 1H), 4.57 (d, J=17.2 Hz, 1H), 4.44 (d, J=17.2 Hz, 1H), 3.60 (s, 2H), 3.02 (d, J=11.2 Hz, 2H), 2.94 (s, 1H), 2.66 (d, J=9.8 Hz, 1H), 2.43 (d, J=4.6 Hz, 1H), 2.14 (t, J=10.1 Hz, 2H), 2.03-1.97 (m, 2H), 1.75-1.66 (m, 4H).

### Example 48. 3-(1-oxo-5-(6-((3-phenylazetidin-1-yl)methyl) imidazo[1,2-a]pyridin-8-yl) isoindolin-2-yl) piperidine-2,6-dione

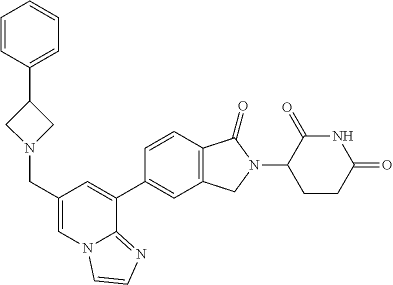 [SMILES: O=C1CCC(N2Cc3cc(-c4cc(CN5CC(c6ccccc6)C5)cn5ccnc45)ccc3C2=O)C(=O)N1]

The title compound was prepared in a manner analogous to Example 1 by reductive amination between 4-phenylazetidine and 8-bromoimidazo[1,2-a]pyridine-6-carbaldehyde

(Intermediate 2) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₃₀H₂₇N₅O₃, 505.21; m/z found, 506.21 [M+H]⁺. ¹H NMR (400 MHz, DMSO-d₆) δ 11.03 (s, 1H), 8.58 (s, 1H), 8.42 (s, 1H), 8.33-8.27 (m, 1H), 8.06 (d, J=1.0 Hz, 1H), 7.86 (d, J=8.0 Hz, 1H), 7.64 (d, J=1.1 Hz, 1H), 7.56 (s, 1H), 7.40-7.30 (m, 4H), 7.23 (t, J=7.0 Hz, 1H), 5.19-5.14 (m, 1H), 4.57 (d, J=17.4 Hz, 1H), 4.43 (d, J=17.4 Hz, 1H), 3.75 (s, 5H), 3.26 (s, 2H), 2.96-2.89 (m, 1H), 2.66-2.63 (m, 1H), 2.47-2.42 (m, 1H), 2.09-2.01 (m, 1H).

### Example 49. 3-(1-oxo-5-(6-((2-phenylpyrrolidin-1-yl)methyl) imidazo[1,2-a]pyridin-8-yl) isoindolin-2-yl) piperidine-2,6-dione

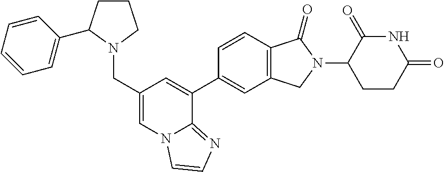 [SMILES: O=C1CCC(N2Cc3cc(-c4cc(CN5CCCC5c5ccccc5)cn5ccnc45)ccc3C2=O)C(=O)N1]

The title compound was prepared in a manner analogous to Example 1 by reductive amination between 2-phenylpyrrolidine and 8-bromoimidazo[1,2-a]pyridine-6-carbaldehyde (Intermediate 2) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₃₁H₂₉N₅O₃, 519.23; m/z found, 520.23 [M+H]⁺. ¹H NMR (400 MHZ, DMSO-d₆) δ 11.01 (s, 1H), 8.48 (s, 1H), 8.31 (s, 1H), 8.19 (d, J=8.2 Hz, 1H), 8.02 (d, J=1.0 Hz, 1H), 7.84 (d, J=8.0 Hz, 1H), 7.60 (d, J=1.0 Hz, 1H), 7.45 (d, J=7.2 Hz, 2H), 7.33 (dd, J=14.2, 6.8 Hz, 3H), 7.22 (d, J=7.2 Hz, 1H), 5.17-5.13 (m, 1H), 4.57 (d, J=17.4 Hz, 1H), 4.44 (d, J=17.4 Hz, 1H), 3.72 (d, J=13.4 Hz, 1H), 3.28 (s, 2H), 3.14 (t, J=7.0 Hz, 1H), 2.98-2.81 (m, 1H), 2.62 (d, J=15.8 Hz, 1H), 2.37 (ddd, J=26.2, 15.4, 8.8 Hz, 2H), 2.24-2.13 (m, 1H), 2.10-1.99 (m, 1H), 1.82-1.78 (m, 2H), 1.69-1.53 (m, 1H).

### Example 50. 3-(1-oxo-5-(7-((2-phenylpyrrolidin-1-yl)methyl)-[1,2,4]triazolo[4,3-a]pyridin-5-yl) isoindolin-2-yl) piperidine-2,6-dione

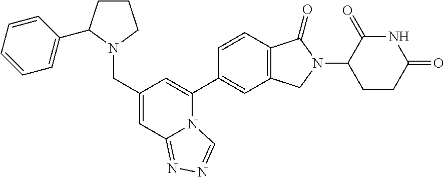 [SMILES: O=C1CCC(N2Cc3cc(-c4cc(CN5CCCC5c5ccccc5)cc5nncn45)ccc3C2=O)C(=O)N1]

The title compound was prepared in a manner analogous to Example 1 by reductive amination between 2-phenylpyrrolidine and 5-chloro-[1,2,4]triazolo[4,3-a]pyridine-7-carbaldehyde (Intermediate 3) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calced for: C₃₀H₂₈N₆O₃ 520.22; m/z found, 521.5 [M+H]⁺. ¹H NMR (400 MHZ, CD3OD) δ 9.11 (s, 1H), 8.04 (d, J=7.6 Hz, 1H), 7.93 (s, 1H), 7.85 (d, J=8.4 Hz, 1H), 7.66 (s, 1H), 7.41 (d, J=7.4 Hz, 2H), 7.24 (t, J=7.6 Hz, 2H), 7.16 (d, J=7.4 Hz, 1H), 6.99 (s, 1H), 5.22 (d, J=13.0 Hz, 1H), 4.64 (d, J=5.8 Hz, 2H), 3.83 (d, J=14.4 Hz, 1H), 3.48 (t, J=10.6 Hz, 3H), 2.94 (s, 1H), 2.83 (s, 1H), 2.71-2.48 (m, 1H), 2.42 (d, J=9.0 Hz, 1H), 2.25 (s, 2H), 1.98-1.83 (m, 3H).

### Example 51:3-(5-(6-(((2-chlorophenethyl)amino)methyl)-1-methyl-1H-benzo[d]imidazol-4-yl)-1-oxoisoindolin-2-yl) piperidine-2,6-dione

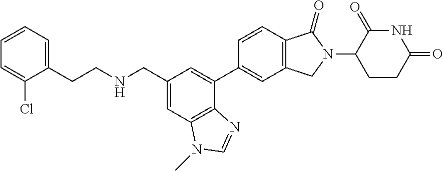 [SMILES: Cn1cnc2c(-c3ccc4c(c3)CN(C3CCC(=O)NC3=O)C4=O)cc(CNCCc3ccccc3Cl)cc21]

The title compound was prepared in a manner analogous to Example 1 by reductive amination between 2-(2-chlorophenyl) ethan-1-amine and 4-bromo-1-methyl-1H-benzo[d]imidazole-6-carbaldehyde (Intermediate 4) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₃₀H₂₈ClN₅O₃, 541.19; m/z found, 542.20 [M+H]⁺. ¹H NMR (400 MHZ, DMSO-d₆) δ 11.01 (s, 1H), 8.35 (s, 1H), 8.26 (d, J=7.4 Hz, 2H), 8.18 (s, 1H), 7.83 (d, J=8.0 Hz, 1H), 7.58 (d, J=3.2 Hz, 2H), 7.38-7.35 (m, 2H), 7.29-7.21 (m, 2H), 5.19-5.14 (m, 1H), 4.56 (d, J=17.2 Hz, 1H), 4.42 (d, J=17.2 Hz, 1H), 3.99 (s, 2H), 3.87 (s, 3H), 2.95-2.90 (m, 2H), 2.86-2.80 (m, 2H), 2.67-2.60 (m, 1H), 2.54-2.52 (m, 1H), 2.46-2.42 (m, 1H), 2.06-1.99 (m, 1H).

### Example 52. 3-(1-oxo-5-(7-(pyrrolidin-1-ylmethyl) imidazo[1,5-a]pyridin-3-yl) isoindolin-2-yl) piperidine-2,6-dione

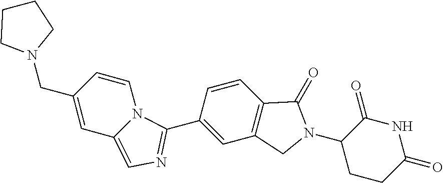 [SMILES: O=C1CCC(N2Cc3cc(-c4ncc5cc(CN6CCCC6)ccn45)ccc3C2=O)C(=O)N1]

The title compound was prepared in a manner analogous to Example 1 by Suzuki coupling of 3-iodo-7-(pyrrolidin-1-ylmethyl) imidazo[1,5-a]pyridine (Intermediate 5) with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₅H₂₅N₅O₃, 443.20; m/z found, 444.2 [M+H]⁺. ¹H NMR (400 MHZ, DMSO-d₆) δ 11.02 (s, 1H), 8.56 (d, J=7.2 Hz, 1H), 8.30 (s, 2H), 8.08 (s, 1H), 7.99 (d, J=8.2 Hz, 1H), 7.87 (d, J=7.8 Hz, 1H), 7.54 (s, 2H), 6.76 (dd, J=7.4, 1.6 Hz, 1H), 5.19-5.14 (m, 1H), 4.56 (d, J=17.6 Hz, 1H), 4.44 (d, J=17.6 Hz, 1H), 3.55 (s, 2H), 2.99-2.86 (m, 1H), 2.60 (s, 1H), 2.52 (s, 4H), 2.43 (s, 1H), 2.05-2.02 (m, 1H), 1.72 (s, 4H).

### Example 53. 3-(1-oxo-5-(6-(pyrrolidin-1-ylmethyl) imidazo[1,5-a]pyridin-3-yl) isoindolin-2-yl) piperidine-2,6-dione

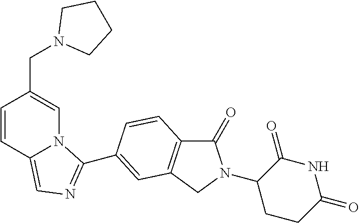 [SMILES: O=C1CCC(N2Cc3cc(-c4ncc5ccc(CN6CCCC6)cn45)ccc3C2=O)C(=O)N1]

The title compound was prepared in a manner analogous to Example 1 by Suzuki coupling of 3-iodo-6-(pyrrolidin-1-ylmethyl) imidazo[1,5-a]pyridine (Intermediate 6) with 3-(1-oxo-5-(4,4,5,5-tetramethyl-1,3,2-dioxaborolan-2-yl) isoindolin-2-yl) piperidine-2,6-dione (Intermediate 13). LC-MS (ESI): mass calcd. for C₂₅H₂₅N₅O₃, 443.20; m/z found, 444.21 [M+H]⁺. ¹H NMR (400 MHZ, DMSO-d₆) δ 11.03 (s, 1H), 8.44 (s, 1H), 8.36 (s, 2H), 8.07 (s, 1H), 7.98 (d, J=8.0 Hz, 1H), 7.89 (d, J=7.8 Hz, 1H), 7.65 (d, J=9.4 Hz, 1H), 7.58 (s, 1H), 6.92 (d, J=9.8 Hz, 1H), 5.19-5.14 (m, 1H), 4.58 (d, J=17.6 Hz, 1H), 4.46 (d, J=17.4 Hz, 1H), 3.56 (s, 2H), 2.98-2.89 (m, 1H), 2.67-2.60 (m, 1H), 2.48-2.33 (m, 5H), 2.11-2.03 (m, 1H), 1.69 (s, 4H).

### Example 54. 3-(1-oxo-5-(8-(pyrrolidin-1-ylmethyl) imidazo[1,5-a]pyridin-5-yl) isoindolin-2-yl) piperidine-2,6-dione

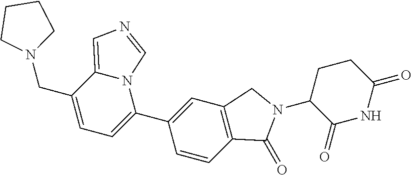 [SMILES: O=C1CCC(N2Cc3cc(-c4ccc(CN5CCCC5)c5cncn45)ccc3C2=O)C(=O)N1]

The title compound was prepared in a manner analogous to Example 1 by reductive amination between pyrrolidine and 5-chloroimidazo[1,5-a]pyridine-8-carbaldehyde (Intermediate 7) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₅H₂₅N₅O₃, 443.20; m/z found, 444.20 [M+H]⁺. ¹H NMR (400 MHZ, DMSO-d₆) δ 11.03 (s, 1H), 8.35 (s, 1H), 8.24 (s, 1H), 8.00 (s, 1H), 7.91 (d, J=7.8 Hz, 1H), 7.85 (d, J=7.6 Hz, 1H), 7.62 (s, 1H), 6.90 (d, J=6.8 Hz, 1H), 6.74 (d, J=6.8 Hz, 1H), 5.19-5.15 (m, 1H), 4.57 (d, J=17.4 Hz, 1H), 4.45 (d, J=17.4 Hz, 1H), 3.80 (s, 2H), 2.99-2.89 (m, 1H), 2.66 (d, J=11.4 Hz, 1H), 2.55 (s, 4H), 2.45-2.39 (m, 1H), 2.08-1.99 (m, 1H), 1.74 (s, 4H).

### Example 55. 3-(5-(1-methyl-4-(pyrrolidin-1-ylmethyl)-1H-pyrrolo[2,3-b]pyridin-6-yl)-1-oxoisoindolin-2-yl) piperidine-2,6-dione

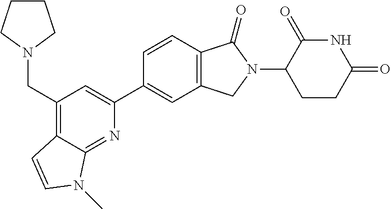 [SMILES: Cn1ccc2c(CN3CCCC3)cc(-c3ccc4c(c3)CN(C3CCC(=O)NC3=O)C4=O)nc21]

### Step A: 6-chloro-1-methyl-4-(pyrrolidin-1-ylmethyl)-1H-pyrrolo[2,3-b]pyridine

To a solution of 6-chloro-1-methyl-1H-pyrrolo[2,3-b]pyridine-4-carbaldehyde (intermediate 8, 200 mg, 1 mmol, 1.0 eq) and pyrrolidine (0.1 mL, 1.2 mmol, 1.2 eq) in DMF (5 mL) was added AcOH (0.06 mL, 1 mmol, 1.0 eq) at room temperature. The reaction mixture was stirred at room temperature for 1 h. NaBH(OAc) 3 (326.7 mg, 1.5 mmol, 1.5 eq) was added to above mixture and the resulting mixture was stirred at room temperature overnight. The reaction mixture was quenched with water (50 mL) and extracted with EtOAc (30 mL×3). The organic layer was washed with brine (30 mL×4), dried over anhydrous Na₂SO₄, filtered and concentrated under reduced pressure. The residue was purified by flash column chromatography on silica gel (PE/EA=1/1) to give 1-({6-chloro-1-methyl-1H-pyrrolo[2,3-b]pyridin-4-yl}methyl) pyrrolidine (200 mg, yield 78%) as a yellow solid. LC-MS (ESI, m/z): mass calcd. for C₁₃H₁₆ClN₃, 249.10; found, 250.10 [M+H]⁺. ¹H NMR (400 MHZ, DMSO-d₆) δ 7.59 (d, J=2.2 Hz, 1H), 7.35 (s, 1H), 6.81-6.68 (m, 1H), 4.33 (s, 2H), 3.79 (s, 3H), 3.06-2.77 (m, 4H), 1.90-1.78 (m, 4H).

### Step B: 3-(5-(1-methyl-4-(pyrrolidin-1-ylmethyl)-1H-pyrrolo[2,3-b]pyridin-6-yl)-1-oxoisoindolin-2-yl) piperidine-2,6-dione

To a solution of 3-[1-oxo-5-(tetramethyl-1,3,2-dioxaborolan-2-yl)-2,3-dihydro-1H-isoindol-2-yl]piperidine-2,6-dione (74.1 mg, 0.2 mmol, 1.0 eq), 1-({6-chloro-1-methyl-1H-pyrrolo[2,3-b]pyridin-4-yl}methyl) pyrrolidine (50 mg, 0.2 mmol, 1.0 eq) in dioxane (2 mL) and H₂O (0.1 mL) was added Pd(dppf)Cl₂ (26 mg, 0.04 mmol, 0.2 eq) and K₃PO₄ (127.5 mg, 0.6 mmol, 3.0 eq) at room temperature. The reaction mixture was stirred at 90° C. for 1 h. After cooled to room temperature, the reaction mixture was quenched with water (5 mL) and concentrated under reduced pressure. The aqueous phase was purified by Prep-HPLC(method A) with YMC-Actus Triart 18C(5 μm, 20×250 mm), and mobile phase of 5-99% ACN in water (0.1% HCOOH) over 10 min and then hold at 100% ACN for 2 min, at a flow rate of 25 mL/min to give 3-(5-{1-methyl-4-[(pyrrolidin-1-yl)methyl]-1H-pyrrolo[2,3-b]pyridin-6-yl}— 1-oxo-2,3-dihydro-1H-isoindol-2-yl) piperidine-2,6-dione formate (5 mg, 5%) as a white solid. LC-MS (ESI, m/z): mass calcd. for C₂₆H₂₇N₅O₃, 457.21; found, 458.2 [M+H]⁺.

¹H NMR (400 MHZ, DMSO-d₆) δ 11.01 (s, 1H), 8.37 (s, 1H), 8.33 (d, J=8.0 Hz, 1H), 8.24 (s, 1H), 7.84 (d, J=8.0 Hz, 1H), 7.75 (s, 1H), 7.55 (d, J=3.2 Hz, 1H), 6.63 (d, J=3.2 Hz, 1H), 5.18-5.13 (m, 1H), 4.57 (d, J=16.8 Hz, 1H), 4.44 (d, J=16.8 Hz, 1H), 3.95 (s, 2H), 3.90 (s, 3H), 3.00-2.88 (m, 1H), 2.64-2.55 (m, 1H), 2.54-2.53 (m, 4H), 2.45-2.34 (m, 1H), 2.09-2.00 (m, 1H), 1.75-1.71 (m, 4H).

### Example 56. 3-(5-(4-(azetidin-1-ylmethyl)-1-methyl-1H-pyrrolo[2,3-b]pyridin-6-yl)-1-oxoisoindolin-2-yl) piperidine-2,6-dione

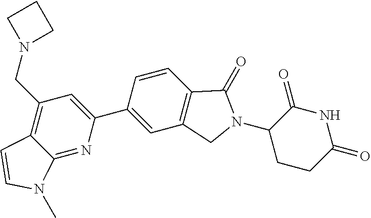 [SMILES: Cn1ccc2c(CN3CCC3)cc(-c3ccc4c(c3)CN(C3CCC(=O)NC3=O)C4=O)nc21]

The title compound was prepared in a manner analogous to Example 55 by reductive amination between azetidine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₅H₂₅N₅O₃, 443.20; m/z found, 444.20 [M+H]⁺. ¹H NMR (400 MHZ, DMSO-d₆) δ 11.02 (s, 1H), 8.40 (s, 1H), 8.35 (d, J=8.0 Hz, 1H), 7.86 (d, J=8.0 Hz, 2H), 7.63 (d, J=3.4 Hz, 1H), 6.72 (d, J=3.4 Hz, 1H), 5.18-5.13 (m, 1H), 4.58 (d, J=17.4 Hz, 1H), 4.44 (d, J=17.4 Hz, 1H), 4.30 (s, 2H), 3.91 (s, 3H), 3.66 (s, 4H), 2.96-2.87 (m, 1H), 2.63 (d, J=17.6 Hz, 1H), 2.46-2.42 (m, 1H), 2.24-2.15 (m, 2H), 2.09-2.00 (m, 1H).

### Example 57. 3-(5-(1-methyl-4-(piperidin-1-ylmethyl)-1H-pyrrolo[2,3-b]pyridin-6-yl)-1-oxoisoindolin-2-yl) piperidine-2,6-dione

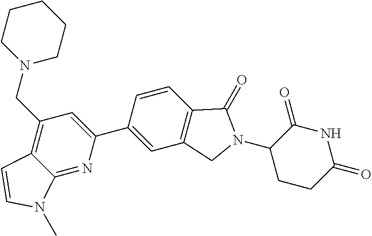 [SMILES: Cn1ccc2c(CN3CCCCC3)cc(-c3ccc4c(c3)CN(C3CCC(=O)NC3=O)C4=O)nc21]

The title compound was prepared in a manner analogous to Example 55 by reductive amination between piperidine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₇H₂₉N₅O₃, 471.23; m/z found, 472.23 [M+H]⁺. ¹H NMR (400 MHZ, DMSO-d₆) δ 11.01 (s, 1H), 8.41-8.29 (m, 2H), 7.84 (d, J=8.2 Hz, 1H), 7.76 (s, 1H), 7.57 (s, 1H), 6.69 (s, 1H), 5.18-5.13 (m, 1H), 4.57 (d, J=17.2 Hz, 1H), 4.44 (d, J=17.2 Hz, 1H), 3.90 (s, 3H), 3.82 (s, 2H), 3.32-3.27 (m, 4H), 3.01-2.89 (m, 1H), 2.65-2.60 (m, 1H), 2.46-2.42 (m, 1H), 2.05 (dd, J=10.8, 5.8 Hz, 1H), 1.62-1.51 (m, 4H), 1.47-1.38 (m, 2H).

### Example 58. 3-(5-(1-methyl-4-(morpholinomethyl)-1H-pyrrolo[2,3-b]pyridin-6-yl)-1-oxoisoindolin-2-yl) piperidine-2,6-dione

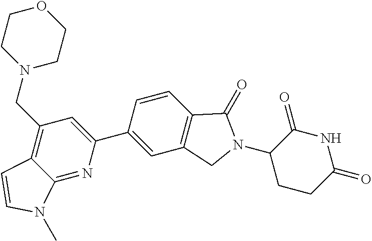 [SMILES: Cn1ccc2c(CN3CCOCC3)cc(-c3ccc4c(c3)CN(C3CCC(=O)NC3=O)C4=O)nc21]

The title compound was prepared in a manner analogous to Example 55 by reductive amination between morpholine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₆H₂₇N₅O₄, 473.21; m/z found, 474.21 [M+H]⁺. ¹H NMR (400 MHZ, DMSO-d₆) δ 11.02 (s, 1H), 8.37 (s, 1H), 8.35-8.29 (m, 1H), 7.84 (d, J=8.0 Hz, 1H), 7.75 (s, 1H), 7.56 (d, J=3.4 Hz, 1H), 6.68 (d, J=3.4 Hz, 1H), 5.18-5.13 (m, 1H), 4.56 (d, J=17.4 Hz, 1H), 4.44 (d, J=17.4 Hz, 1H), 3.90 (s, 3H), 3.83 (s, 2H), 3.64-3.56 (m, 4H), 2.99-2.87 (m, 1H), 2.63 (d, J=16.4 Hz, 1H), 2.45 (s, 5H), 2.07-2.02 (m, 1H).

### Example 59. 3-(1-oxo-5-(4-(pyrrolidin-1-ylmethyl)-1H-pyrrolo[2,3-b]pyridin-6-yl) isoindolin-2-yl) piperidine-2,6-dione

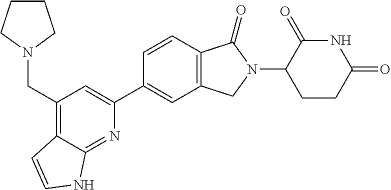 [SMILES: O=C1CCC(N2Cc3cc(-c4cc(CN5CCCC5)c5cc[nH]c5n4)ccc3C2=O)C(=O)N1]

The title compound was prepared in a manner analogous to Example 55 by reductive amination between pyrrolidine and 6-chloro-1-1H-pyrrolo[2,3-b]pyridine-4-carbaldehyde (Intermediate 9) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₅H₂₅N₅O₃, 443.20; m/z found, 444.21 [M+H]⁺. ¹H NMR (400 MHZ, DMSO-d₆) δ 11.95 (s, 1H), 11.02 (s, 1H), 8.47-7.97 (m, 3H), 7.84 (d, J=7.8 Hz, 1H), 7.61 (s, 1H), 6.77 (s, 1H), 5.18-5.13 (m, 1H), 4.85-4.25 (m, 4H), 3.31-2.70 (m, 5H), 2.63 (d, J=16.2 Hz, 1H), 2.47-2.38 (m, 1H), 2.64-1.91 (m, 5H).

### Example 60. 3-(5-(4-((4,4-difluoropiperidin-1-yl)methyl)-1-methyl-1H-pyrrolo[2,3-b]pyridin-6-yl)-1-oxoisoindolin-2-yl) piperidine-2,6-dione

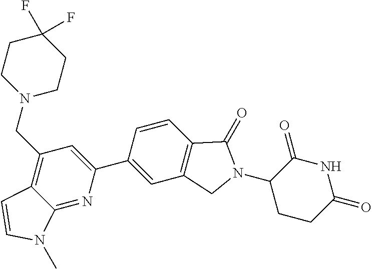 [SMILES: Cn1ccc2c(CN3CCC(F)(F)CC3)cc(-c3ccc4c(c3)CN(C3CCC(=O)NC3=O)C4=O)nc21]

The title compound was prepared in a manner analogous to Example 55 by reductive amination between 4,4-difluoropiperidine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₇H₂₇F₂N₅O₃, 507.21; m/z found, 508.21 [M+H]⁺. ¹H NMR (400 MHZ, DMSO-d₆) δ 11.02 (s, 1H), 8.38 (s, 1H), 8.35 (d, J=8.2 Hz, 1H), 7.84 (d, J=8.0 Hz, 1H), 7.77 (s, 1H), 7.57 (d, J=3.4 Hz, 1H), 6.69 (d, J=3.4 Hz, 1H), 5.18-5.13 (m, 1H), 4.57 (d, J=17.2 Hz, 1H), 4.44 (d, J=17.2 Hz, 1H), 3.92 (s, 3H), 3.90 (s, 2H), 3.02-2.85 (m, 1H), 2.64-2.58 (m, 5H), 2.47-2.34 (m, 1H), 2.08-1.93 (m, 5H).

### Example 61.3-(5-(1-methyl-4-((4-(methylsulfonyl) piperazin-1-yl)methyl)-1H-pyrrolo[2,3-b]pyridin-6-yl)-1-oxoisoindolin-2-yl) piperidine-2,6-dione

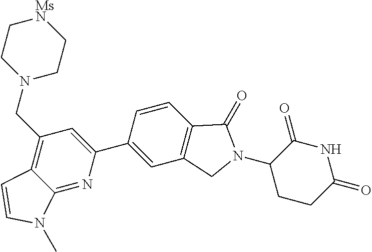

The title compound was prepared in a manner analogous to Example 55 by reductive amination between 1-(methylsulfonyl) piperazine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₇H₃₀N₆O₅S, 550.2; m/z found, 551.21 [M+H]⁺. ¹H NMR (400 MHZ, DMSO-d₆) δ 11.02 (s, 1H), 8.39 (s, 1H), 8.34-8.32 (m, 1H), 7.84 (d, J=8.0 Hz, 1H), 7.75 (s, 1H), 7.57 (d, J=3.4 Hz, 1H), 6.68 (d, J=3.4 Hz, 1H), 5.18-5.14 (m, 1H), 4.57 (d, J=17.2 Hz, 1H), 4.44 (d, J=17.2 Hz, 1H), 3.91 (s, 5H), 3.14-3.13 (m, 4H), 2.99-2.92 (m, 1H), 2.91 (s, 3H), 2.65-2.54 (m, 5H), 2.46-2.37 (m, 1H), 2.05-2.03 (m, 1H).

### Example 62. 3-(5-(1-methyl-4-(thiomorpholinomethyl)-1H-pyrrolo[2,3-b]pyridin-6-yl)-1-oxoisoindolin-2-yl) piperidine-2,6-dione

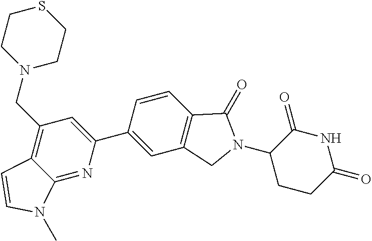 [SMILES: Cn1ccc2c(CN3CCSCC3)cc(-c3ccc4c(c3)CN(C3CCC(=O)NC3=O)C4=O)nc21]

The title compound was prepared in a manner analogous to Example 55 by reductive amination between thiomorpholine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₆H₂₇N₅O₃S, 489.18; m/z found, 490.18 [M+H]⁺. ¹H NMR (400 MHZ, DMSO-d₆) δ 11.01 (s, 1H), 8.38 (s, 1H), 8.34 (d, J=8.0 Hz, 1H), 7.84 (d, J=8.0 Hz, 1H), 7.74 (s, 1H), 7.56 (d, J=3.4 Hz, 1H), 6.68 (d, J=3.4 Hz, 1H), 5.18-5.13 (m, 1H), 4.57 (d, J=17.2 Hz, 1H), 4.44 (d, J=17.2 Hz, 1H), 3.90 (s, 3H), 3.87 (s, 2H), 3.00-2.89 (m, 1H), 2.72 (d, J=4.0 Hz, 4H), 2.65-2.60 (m, 5H), 2.47-2.37 (m, 1H), 2.06-2.03 (m, 1H).

### Example 63. 3-(5-(1-methyl-4-((tetrahydro-1H-furo[3,4-c]pyrrol-5 (3H)-yl)methyl)-1H-pyrrolo[2,3-b]pyridin-6-yl)-1-oxoisoindolin-2-yl) piperidine-2,6-dione

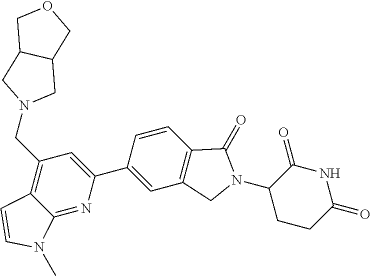 [SMILES: Cn1ccc2c(CN3CC4COCC4C3)cc(-c3ccc4c(c3)CN(C3CCC(=O)NC3=O)C4=O)nc21]

The title compound was prepared in a manner analogous to Example 55 by reductive amination between hexahydro-1H-furo[3,4-c]pyrrole and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₈H₂₉N₅O₄, 499.22; m/z found, 500.22 [M+H]⁺. ¹H NMR (400 MHZ, DMSO-d₆) δ 11.01 (s, 1H), 8.36 (s, 1H), 8.33 (d, J=8.0 Hz, 1H), 8.14 (s, 1H), 7.84 (d, J=8.0 Hz, 1H), 7.74 (s, 1H), 7.56 (d, J=3.4 Hz, 1H), 6.67 (d, J=3.4 Hz, 1H), 5.18-5.13 (m, 1H), 4.57 (d, J=17.2 Hz, 1H), 4.44 (d, J=17.2 Hz, 1H), 3.93 (s, 2H), 3.90 (s, 3H), 3.79-3.71 (m, 2H), 3.41 (d, J=5.0 Hz, 2H), 2.94-2.89 (m, 1H), 2.73 (s, 2H), 2.66 (d, J=9.2 Hz, 1H), 2.60 (s, 2H), 2.44 (d, J=8.6 Hz, 3H), 2.10-1.99 (m, 1H).

### Example 64. 3-(5-(4-((6-azaspiro[2.5]octan-6-yl)methyl)-1-methyl-1H-pyrrolo[2,3-b]pyridin-6-yl)-1-oxoisoindolin-2-yl) piperidine-2,6-dione

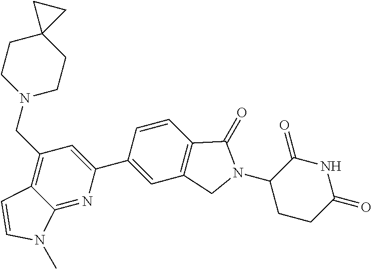 [SMILES: Cn1ccc2c(CN3CCC4(CC3)CC4)cc(-c3ccc4c(c3)CN(C3CCC(=O)NC3=O)C4=O)nc21]

The title compound was prepared in a manner analogous to Example 55 by reductive amination between 6-azaspiro[2.5]octane and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₉H₃₁N₅O₃, 497.24; m/z found, 498.24 [M+H]⁺. ¹H NMR (400 MHZ, DMSO-d₆) δ 11.04 (s, 1H), 10.09 (s, 1H), 8.39 (s, 1H), 8.35 (d, J=8.2 Hz, 1H), 8.06 (s, 1H), 7.90 (d, J=8.2 Hz, 1H), 7.75 (d, J=3.6 Hz, 1H), 6.89 (d, J=3.6 Hz, 1H), 5.19-5.14 (m, 1H), 4.73 (d, J=4.4 Hz, 2H), 4.59 (d, J=17.4 Hz, 1H), 4.46 (d, J=17.6 Hz, 1H), 3.95 (s, 3H), 3.46 (d, J=11.0 Hz, 2H), 3.18-3.15 (m, 2H), 3.01-2.89 (m, 1H), 2.64 (d, J=17.0 Hz, 1H), 2.48-2.34 (m, 1H), 2.08 (dt, J=10.2, 9.0 Hz, 3H), 1.14 (d, J=14.0 Hz, 2H), 0.43-0.39 (m, 4H).

### Example 65. 3-(5-(4-((3,4-dihydroisoquinolin-2 (1H)-yl)methyl)-1-methyl-1H-pyrrolo[2,3-b]pyridin-6-yl)-1-oxoisoindolin-2-yl) piperidine-2,6-dione

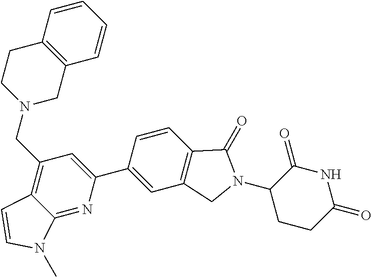 [SMILES: Cn1ccc2c(CN3CCc4ccccc4C3)cc(-c3ccc4c(c3)CN(C3CCC(=O)NC3=O)C4=O)nc21]

The title compound was prepared in a manner analogous to Example 55 by reductive amination between 1,2,3,4-tetrahydroisoquinoline and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₃₁H₂₉N₅O₃, 519.23; m/z found, 520.23 [M+H]⁺. ¹H NMR (400 MHZ, DMSO-d₆) δ 11.02 (s, 1H), 8.38 (s, 1H), 8.34 (d, J=8.2 Hz, 1H), 7.86-7.81 (m, 2H), 7.56 (d, J=3.4 Hz, 1H), 7.13-7.06 (m, 3H), 7.01 (d, J=7.2 Hz, 1H), 6.68 (d, J=3.4 Hz, 1H), 5.17-5.12 (m, 1H), 4.57 (d, J=17.2 Hz, 1H), 4.44 (d, J=17.2 Hz, 1H), 4.03 (s, 2H), 3.92 (s, 3H), 3.66 (s, 2H), 2.98-2.90 (m, 1H), 2.85 (d, J=5.2 Hz, 2H), 2.78 (d, J=5.2 Hz, 2H), 2.65-2.61 (m, 1H), 2.46-2.37 (m, 1H), 2.07-2.04 (m, 1H).

### Example 66. 3-(5-(4-((3-azabicyclo[3.1.0]hexan-3-yl)methyl)-1-methyl-1H-pyrrolo[2,3-b]pyridin-6-yl)-1-oxoisoindolin-2-yl) piperidine-2,6-dione

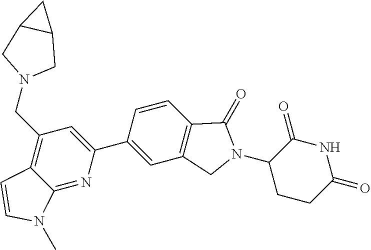 [SMILES: Cn1ccc2c(CN3CC4CC4C3)cc(-c3ccc4c(c3)CN(C3CCC(=O)NC3=O)C4=O)nc21]

The title compound was prepared in a manner analogous to Example 55 by reductive amination between 3-azabicyclo[3.1.0]hexane and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calced for: C₂₇H₂₇N₅O₃ 469.21; m/z found, 470.1 [M+H]⁺. ¹H NMR (400 MHZ, DMSO-d₆) δ 11.02 (s, 1H), 8.36 (s, 1H), 8.32 (d, J=8.0 Hz, 1H), 8.14 (s, 1H), 7.84 (d, J=8.0 Hz, 1H), 7.69 (s, 1H), 7.55 (d, J=3.4 Hz, 1H), 6.59 (d, J=3.4 Hz, 1H), 5.18-5.13 (m, 1H), 4.57 (d, J=17.4 Hz, 1H), 4.44 (d, J=17.4 Hz, 1H), 3.96 (s, 2H), 3.90 (s, 3H), 2.96-2.90 (m, 3H), 2.63 (d, J=17.2 Hz, 1H), 2.48-2.36 (m, 3H), 2.11-1.98 (m, 1H), 1.39 (s, 2H), 0.74 (d, J=3.8 Hz, 1H), 0.43-0.29 (m, 1H).

### Example 67. 3-(5-(4-(isoindolin-2-ylmethyl)-1-methyl-1H-pyrrolo[2,3-b]pyridin-6-yl)-1-oxoisoindolin-2-yl) piperidine-2,6-dione

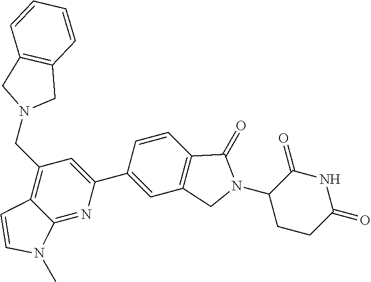 [SMILES: Cn1ccc2c(CN3Cc4ccccc4C3)cc(-c3ccc4c(c3)CN(C3CCC(=O)NC3=O)C4=O)nc21]

The title compound was prepared in a manner analogous to Example 55 by reductive amination between isoindoline and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calced for: C₃₀H₂₇N₅O₃ 505.21; m/z found, 506.2 [M+H]⁺. ¹H NMR (400 MHZ, DMSO-d₆) δ 11.01 (s, 1H), 8.39 (s, 1H), 8.36 (d, J=8.4 Hz, 1H), 7.84 (d, J=8.2 Hz, 2H), 7.58 (s, 1H), 7.22 (d, J=12.2 Hz, 4H), 6.69 (s, 1H), 5.16-5.12 (m, H), 4.56 (d, J=17.4 Hz, 1H), 4.43 (d, J=17.4 Hz, 1H), 4.26 (s, 2H), 3.97 (s, 4H), 3.92 (s, 3H), 2.94-2.89 (m, 1H), 2.64-2.60 (m, 1H), 2.44-2.39 (m, 1H), 2.05-2.02 (m, 1H).

### Example 68. 3-(5-(4-((1,1-dioxidothiomorpholino)methyl)-1-methyl-1H-pyrrolo[2,3-b]pyridin-6-yl)-1-oxoisoindolin-2-yl) piperidine-2,6-dione

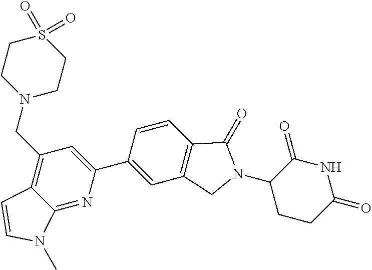 [SMILES: Cn1ccc2c(CN3CCS(=O)(=O)CC3)cc(-c3ccc4c(c3)CN(C3CCC(=O)NC3=O)C4=O)nc21]

The title compound was prepared in a manner analogous to Example 55 by reductive amination between thiomorpholine 1,1-dioxide and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calced for: C₂₆H₂₇N₅O₅S 521.17; m/z found, 522.4 [M+H]⁺. ¹H NMR (400 MHZ, DMSO-d₆) δ 11.02 (s, 1H), 8.48-8.24 (m, 2H), 7.91-7.76 (m, 2H), 7.58 (d, J=3.0 Hz, 1H), 6.73 (d, J=3.0 Hz, 1H), 5.18-5.14 (m, 1H), 4.57 (d, J=17.2 Hz, 1H), 4.44 (d, J=17.2 Hz, 1H), 4.05 (s, 2H), 3.91 (s, 3H), 3.17 (s, 4H), 2.98 (s, 4H), 2.92 (dd, J=14.0, 5.0 Hz, 1H), 2.65-2.64 (m, 1H), 2.43-2.40 (s, 1H), 2.06-2.04 (m, 1H).

### Example 69. 3-(5-(4-((7-azaspiro[3.5]nonan-7-yl)methyl)-1-methyl-1H-pyrrolo[2,3-b]pyridin-6-yl)-1-oxoisoindolin-2-yl) piperidine-2,6-dione

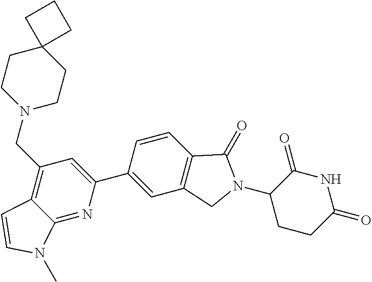 [SMILES: Cn1ccc2c(CN3CCC4(CCC4)CC3)cc(-c3ccc4c(c3)CN(C3CCC(=O)NC3=O)C4=O)nc21]

The title compound was prepared in a manner analogous to Example 55 by reductive amination between 7-azaspiro[3.5]nonane and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₃₀H₃₃N₅O₃, 511.26; m/z found, 512.27 [M+H]⁺. ¹H NMR (400 MHZ, DMSO-d₆) δ 11.03 (s, 1H), 9.69 (s, 1H), 8.38 (s, 1H), 8.34 (d, J=8.2 Hz, 1H), 8.02 (s, 1H), 7.90 (d, J=8.0 Hz, 1H), 7.75 (d, J=3.4 Hz, 1H), 6.86 (d, J=3.4 Hz, 1H), 5.18-5.14 (m, 1H), 4.66 (d, J=4.8 Hz, 2H), 4.59 (d, J=17.2 Hz, 1H), 4.46 (d, J=17.2 Hz, 1H), 3.95 (s, 3H), 3.32 (d, J=11.2 Hz, 2H), 3.10-3.02 (m, 2H), 2.99-2.89 (m, 1H), 2.64 (d, J=16.4 Hz, 1H), 2.47-2.43 (m, 1H), 2.09-2.02 (m, 1H), 1.92-1.89 (m, 2H), 1.85 (s, 4H), 1.69-1.63 (m, 4H).

### Example 70. 3-(5-(4-((3-oxa-7-azabicyclo[3.3.1]nonan-7-yl)methyl)-1-methyl-1H-pyrrolo[2,3-b]pyridin-6-yl)-1-oxoisoindolin-2-yl) piperidine-2,6-dione

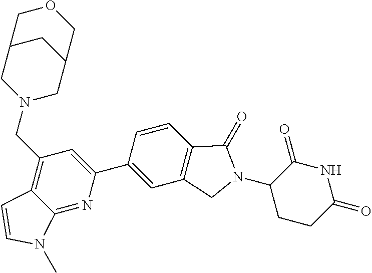 [SMILES: Cn1ccc2c(CN3CC4COCC(C4)C3)cc(-c3ccc4c(c3)CN(C3CCC(=O)NC3=O)C4=O)nc21]

The title compound was prepared in a manner analogous to Example 55 by reductive amination between 3-oxa-7-azabicyclo[3.3.1]nonane and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₉H₃₁N₅O₄, 513.24; m/z found, 514.25 [M+H]⁺. ¹H NMR (400 MHZ, DMSO-d₆) δ 11.03 (s, 1H), 8.92 (s, 1H), 8.38 (s, 1H), 8.33 (d, J=8.2 Hz, 1H), 8.18 (s, 1H), 7.92 (d, J=8.0 Hz, 1H), 7.76 (d, J=3.4 Hz, 1H), 6.89 (d, J=3.4 Hz, 1H), 5.19-5.15 (m, 1H), 4.61 (d, J=5.4 Hz, 2H), 4.58 (d, J=17.4 Hz, 1H), 4.47 (d, J=17.4 Hz, 1H), 4.00 (d, J=11.0 Hz, 2H), 3.95 (s, 3H), 3.68 (d, J=11.4 Hz, 2H), 3.60 (d, J=11.4 Hz, 2H), 3.40 (t, J=10.6 Hz, 2H), 3.00-2.90 (m, 1H), 2.66-2.61 (m, 1H), 2.47-2.43 (m, 1H), 2.09-2.03 (m, 1H), 2.01 (s, 2H), 1.88-1.82 (m, 2H).

### Example 71. 3-(5-(1-methyl-4-((2-phenylazetidin-1-yl)methyl)-1H-pyrrolo[2,3-b]pyridin-6-yl)-1-oxoisoindolin-2-yl) piperidine-2,6-dione

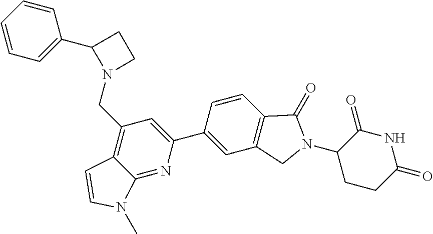 [SMILES: Cn1ccc2c(CN3CCC3c3ccccc3)cc(-c3ccc4c(c3)CN(C3CCC(=O)NC3=O)C4=O)nc21]

The title compound was prepared in a manner analogous to Example 55 by reductive amination between 2-phenylazetidine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₃₁H₂₉N₅O₃, 519.23; m/z found, 519.23 [M+H]⁺. ¹H NMR (400 MHZ, DMSO-d₆) δ 11.02 (s, 1H), 8.21 (d, J=1.6 Hz, 2H), 7.82 (d, J=8.4 Hz, 1H), 7.64 (s, 1H), 7.55 (d, J=3.2 Hz, 1H), 7.50 (d, J=7.2 Hz, 2H), 7.35 (t, J=7.4 Hz, 2H), 7.26 (d, J=7.2 Hz, 1H), 6.58 (d, J=3.2 Hz, 1H), 5.18-5.14 (m, 1H), 4.57 (d, J=17.4 Hz, 1H), 4.44 (d, J=17.4 Hz, 1H), 4.25 (t, J=8.0 Hz, 1H), 4.07 (d, J=14.2 Hz, 1H), 3.87 (d, J=10.2 Hz, 4H), 3.33 (s, 1H), 3.00-2.94 (m, 2H), 2.63 (d, J=17.0 Hz, 1H), 2.47-2.42 (m, 1H), 2.37-2.32 (m, 1H), 2.07-2.02 (m, 2H).

### Example 72. 3-(5-(4-((hexahydrocyclopenta[c]pyrrol-2 (1H)-yl)methyl)-1-methyl-1H-pyrrolo[2,3-b]pyridin-6-yl)-1-oxoisoindolin-2-yl) piperidine-2,6-dione

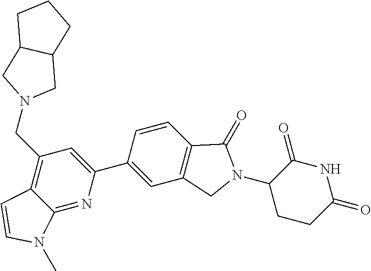 [SMILES: Cn1ccc2c(CN3CC4CCCC4C3)cc(-c3ccc4c(c3)CN(C3CCC(=O)NC3=O)C4=O)nc21]

The title compound was prepared in a manner analogous to Example 55 by reductive amination between octahydrocyclopenta[c]pyrrole and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₉H₃₁N₅O₃, 497.24; m/z found, 498.24 [M+H]⁺. ¹H NMR (400 MHZ, DMSO-d₆) δ 11.02 (s, 1H), 8.41 (s, 1H), 8.36 (d, J=8.0 Hz, 1H), 8.10 (s, 1H), 7.85 (d, J=8.0 Hz, 1H), 7.64 (d, J=2.4 Hz, 1H), 6.77 (s, 1H), 5.18-5.13 (m, 1H), 4.57 (d, J=17.4 Hz, 1H), 4.43 (d, J=17.4 Hz, 1H)_{4.41} (s, 2H), 3.92 (s, 3H), 3.24-3.03 (m, 2H), 2.96-2.87 (m, 1H), 2.64-2.60 (m, 5H), 2.46-2.42 (m, 1H), 2.11-1.95 (m, 1H), 1.72-1.41 (m, 6H).

### Example 73. 3-(5-(4-((2-oxa-7-azaspiro[3.5]nonan-7-yl)methyl)-1-methyl-1H-pyrrolo[2,3-b]pyridin-6-yl)-1-oxoisoindolin-2-yl) piperidine-2,6-dione

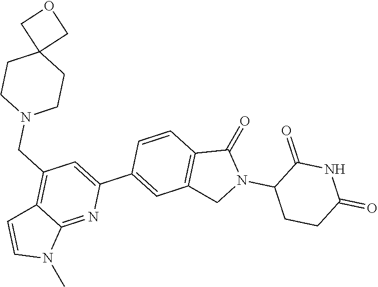 [SMILES: Cn1ccc2c(CN3CCC4(CC3)COC4)cc(-c3ccc4c(c3)CN(C3CCC(=O)NC3=O)C4=O)nc21]

The title compound was prepared in a manner analogous to Example 55 by reductive amination between 2-oxa-7-azaspiro[3.5]nonane and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calced for: C₂₉H₃₁N₅O₄ 513.24; m/z found, 514.4 [M+H]⁺. ¹H NMR (400 MHZ, DMSO-d₆) δ 11.02 (s, 1H), 8.37 (s, 1H), 8.32 (d, J=8.0 Hz, 1H), 8.14 (s, 1H), 7.84 (d, J=8.0 Hz, 1H), 7.73 (s, 1H), 7.55 (d, J=3.4 Hz, 1H), 6.66 (d, J=3.4 Hz, 1H), 5.18-5.13 (m, 1H), 4.57 (d, J=17.4 Hz, 1H), 4.44 (d, J=17.4 Hz, 1H), 4.28 (s, 4H), 3.90 (s, 3H), 3.80 (s, 2H), 3.00-2.88 (m, 1H), 2.65-2.61 (m, 1H), 2.46-2.31 (m, 5H), 2.10-1.99 (m, 1H), 1.81 (s, 4H).

### Example 74. 3-(5-(1-ethyl-4-(pyrrolidin-1-ylmethyl)-1H-pyrrolo[2,3-b]pyridin-6-yl)-1-oxoisoindolin-2-yl) piperidine-2,6-dione

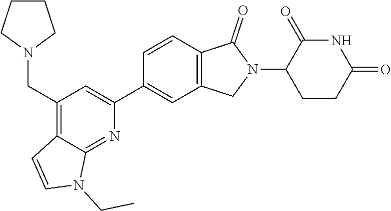 [SMILES: CCn1ccc2c(CN3CCCC3)cc(-c3ccc4c(c3)CN(C3CCC(=O)NC3=O)C4=O)nc21]

The title compound was prepared in a manner analogous to Example 55 by reductive amination between pyrrolidine and 6-chloro-1-ethyl-1H-pyrrolo[2,3-b]pyridine-4-carbaldehyde (Intermediate 10) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calcd. for C₂₇H₂₉N₅O₃, 471.23; m/z found, 472.23 [M+H]⁺. ¹H NMR (400 MHZ, DMSO-d₆) δ 11.04 (s, 1H), 8.39 (s, 1H), 8.35 (d, J=8.1 Hz, 1H), 8.24 (s, 1.5H), 7.99 (s, 1H), 7.86 (d, J=8.0 Hz, 1H), 7.70 (d, J=3.5 Hz, 1H), 6.74 (d, J=3.5 Hz, 1H), 5.19-5.14 (m, 1H), 4.58 (d, J=17.4 Hz, 1H), 4.49-4.36 (m, 3H), 4.31 (s, 2H), 3.04-2.77 (m, 5H), 2.68-2.60 (m, 1H), 2.47-2.44 (m, 1H), 2.07-2.05 (m, 1H), 1.85 (s, 4H), 1.46 (t, J=7.2 Hz, 3H).

### Example 75. 3-(4-fluoro-5-(1-methyl-4-(pyrrolidin-1-ylmethyl)-1H-pyrrolo[2,3-b]pyridin-6-yl)-1-oxoisoindolin-2-yl) piperidine-2,6-dione

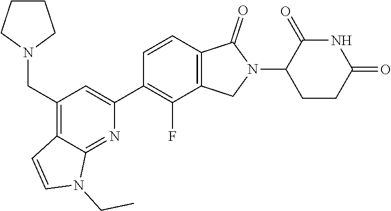 [SMILES: CCn1ccc2c(CN3CCCC3)cc(-c3ccc4c(c3F)CN(C3CCC(=O)NC3=O)C4=O)nc21]

The title compound was prepared in a manner analogous to Example 55 by reductive amination between pyrrolidine and 6-chloro-1-methyl-1H-pyrrolo[2,3-b]pyridine-4-carbaldehyde (Intermediate 8) followed by Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14).

LC-MS (ESI): mass calcd. for C₂₆H₂₆FN₅O₃, 475.20; m/z found, 476.20 [M+H]⁺. ¹H NMR (400 MHZ, DMSO-d₆) δ 11.04 (s, 1H), 8.21 (t, J=7.4 Hz, 1H), 7.73 (d, J=7.8 Hz, 2H), 7.65 (s, 1H), 6.72 (s, 1H), 5.19-5.14 (m, 1H), 4.67 (d, J=17.4 Hz, 1H), 4.50 (d, J=17.4 Hz, 1H), 4.19 (s, 2H), 3.89 (s, 3H), 3.01-2.58 (m, 6H), 2.47-2.46 (m, 1H), 2.07-2.03 (m, 1H), 1.80 (s, 4H).

### Example 76. 3-(1-oxo-5-(7-(pyrrolidin-1-ylmethyl)-1H-pyrrolo[3,2-b]pyridin-5-yl) isoindolin-2-yl) piperidine-2,6-dione

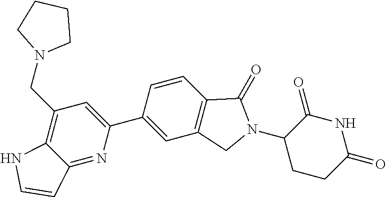 [SMILES: O=C1CCC(N2Cc3cc(-c4cc(CN5CCCC5)c5[nH]ccc5n4)ccc3C2=O)C(=O)N1]

The title compound was prepared in a manner analogous to Example 55 by reductive amination between pyrrolidine and 5-chloro-1H-pyrrolo[3,2-b]pyridine-7-carbaldehyde (Intermediate 11) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calced for C₁₀H₉ClN₂O₂ 443.5; m/z found, 444.3 [M+H]⁺. ¹H NMR (400 MHZ, DMSO-d₆) δ 11.30 (s, 1H), 11.01 (s, 1H), 8.33 (s, 1H), 8.25 (d, J=8.0 Hz, 1H), 8.18 (s, 1H), 7.80 (d, J=8.0 Hz, 1H), 7.73 (d, J=3.6 Hz, 1H), 7.66 (s, 1H), 6.64 (d, J=1.6 Hz, 1H), 5.17-5.12 (m, 1H), 4.55 (d, J=17.4 Hz, 1H), 4.42 (d, J=17.4 Hz, 1H), 3.98 (s, 2H), 2.96-2.88 (m, 1H), 2.69-2.53 (m, 5H), 2.44-2.38 (m, 1H), 2.09-2.00 (m, 1H), 1.75 (s, 4H).

### Example 77. 3-(5-(1-methyl-7-(pyrrolidin-1-ylmethyl)-1H-pyrrolo[3,2-b]pyridin-5-yl)-1-oxoisoindolin-2-yl) piperidine-2,6-dione

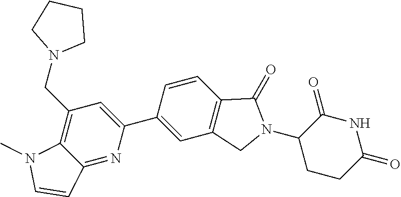 [SMILES: Cn1ccc2nc(-c3ccc4c(c3)CN(C3CCC(=O)NC3=O)C4=O)cc(CN3CCCC3)c21]

The title compound was prepared in a manner analogous to Example 55 by reductive amination between pyrrolidine and 5-chloro-1-methyl-1H-pyrrolo[3,2-b]pyridine-7-carbaldehyde (Intermediate 12) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calced for C₂₆H₂₇N₅O₃ 456.4; m/z found, 458.4 [M+H]⁺. ¹H NMR (400 MHZ, DMSO-d₆) δ 11.01 (s, 1H), 8.35 (s, 1H), 8.26 (d, J=8.0 Hz, 1H), 8.17 (s, 1H), 7.80 (d, J=8.0 Hz, 1H), 7.71 (s, 1H), 7.59 (d, J=3.2 Hz, 1H), 6.61 (d, J=3.2 Hz, 1H), 5.17-5.12 (m, 1H), 4.55 (d, J=17.4 Hz, 1H), 4.42 (d, J=17.4 Hz, 1H), 4.14 (s, 3H), 4.02 (s, 2H), 3.00-2.87 (m, 1H), 2.64-2.59 (m, 1H), 2.55-2.53 (m, 4H), 2.48-2.37 (m, 1H), 2.07-1.98 (m, 1H), 1.73-1.71 (m, 4H).

### Example 78:3-(1-oxo-5-(4-(pyrrolidin-1-ylmethyl) quinolin-2-yl) isoindolin-2-yl) piperidine-2,6-dione

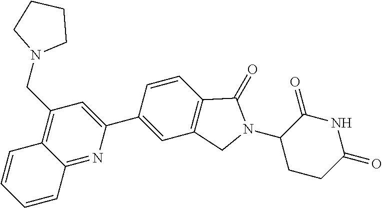 [SMILES: O=C1CCC(N2Cc3cc(-c4cc(CN5CCCC5)c5ccccc5n4)ccc3C2=O)C(=O)N1]

The title compound was prepared in a manner analogous to Example 55 by reductive amination between pyrrolidine and 2-chloroquinoline-4-carbaldehyde (Intermediate 22) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calced for C₂₆H₂₇N₄O₃ 454.5; m/z found, 455.4 [M+H]⁺. ¹H NMR (400 MHZ, DMSO) δ 11.03 (s, 1H), 8.51 (s, 1H), 8.43 (d, J=8.1 Hz, 1H), 8.33 (d, J=8.3 Hz, 1H), 8.19 (s, 1H), 8.16-8.10 (m, 2H), 7.91 (d, J=8.0 Hz, 1H), 7.81 (t, J=7.6 Hz, 1H), 7.64 (t, J=7.6 Hz, 1H), 5.18 (dd, J=13.3, 5.1 Hz, 1H), 4.54 (dd, J=50.3, 17.4 Hz, 2H), 4.16 (s, 2H), 2.99-2.89 (m, 1H), 2.63 (d, J=20.1 Hz, 5H), 2.45 (dd, J=13.1, 4.3 Hz, 1H), 2.10-2.02 (m, 1H), 1.75 (s, 4H).

### Example 79:3-(1-oxo-5-(4-(pyrrolidin-1-ylmethyl)-5,6,7,8-tetrahydroquinolin-2-yl) isoindolin-2-yl) piperidine-2,6-dione

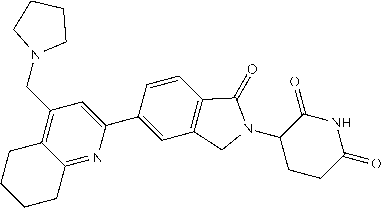 [SMILES: O=C1CCC(N2Cc3cc(-c4cc(CN5CCCC5)c5c(n4)CCCC5)ccc3C2=O)C(=O)N1]

The title compound was prepared in a manner analogous to Example 55 by Suzuki coupling with 2-chloro-4-(pyrrolidin-1-ylmethyl)-5,6,7,8-tetrahydroquinoline (Intermediate 23) and 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calced for C₂₇H₃₀N₄O₃ 458.2; m/z found, 459.1 [M+H]⁺. ¹H NMR (400 MHZ, DMSO) δ 11.01 (s, 1H), 8.25 (s, 1H), 8.18 (d, J=8.1 Hz, 1H), 8.15 (s, 1H), 7.81 (d, J=8.0 Hz, 1H), 7.78 (s, 1H), 5.14 (dd, J=13.2, 5.1 Hz, 1H), 4.54 (d, J=17.4 Hz, 1H), 4.41 (d, J=17.3 Hz, 1H), 3.62 (s, 2H), 2.98-2.87 (m, 3H), 2.80 (d, J=6.1 Hz, 2H), 2.62 (d, J=17.5 Hz, 1H), 2.52 (s, 4H), 2.45-2.31 (m, 1H), 2.09-1.98 (m, 1H), 1.87-1.77 (m, 4H), 1.74 (s, 4H).

### Example 80:3-(1-oxo-5-(4-(pyrrolidin-1-ylmethyl)-6,7-dihydro-5H-cyclopenta[b]pyridin-2-yl) isoindolin-2-yl) piperidine-2,6-dione

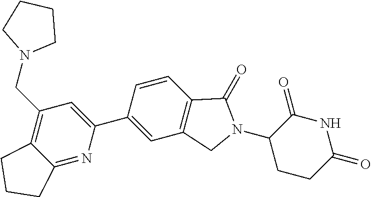 [SMILES: O=C1CCC(N2Cc3cc(-c4cc(CN5CCCC5)c5c(n4)CCC5)ccc3C2=O)C(=O)N1]

The title compound was prepared in a manner analogous to Example 55 by reductive amination between pyrrolidine and 2-chloro-6,7-dihydro-5H-cyclopenta[b]pyridine-4-carbaldehyde (Intermediate 24) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13).

LC-MS (ESI): mass calced for C₂₆H₂₈N₄O₃ 444.2; m/z found, 445.3 [M+H]⁺. ¹H NMR (400 MHZ, DMSO) δ 11.01 (s, 1H), 8.27 (s, 1H), 8.20 (s, 1H), 8.17 (d, J=1.6 Hz, 1H), 7.81 (d, J=8.0 Hz, 1H), 7.74 (s, 1H), 5.15 (dd, J=13.3, 5.1 Hz, 1H), 4.54 (d, J=17.4 Hz, 1H), 4.42 (d, J=17.3 Hz, 1H), 3.64 (s, 2H), 2.96 (ddd, J=25.0, 16.5, 6.5 Hz, 5H), 2.67-2.58 (m, 1H), 2.52 (d, J=1.6 Hz, 4H), 2.47-2.37 (m, 1H), 2.15-1.99 (m, 3H), 1.74 (s, 4H).

### Example 81:3-(5-(1-methyl-4-((4-methylpiperidin-1-yl)methyl)-1H-pyrrolo[2,3-b]pyridin-6-yl)-1-oxoisoindolin-2-yl) piperidine-2,6-dione

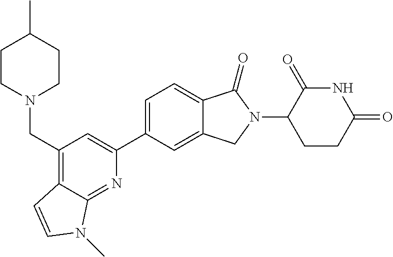 [SMILES: CC1CCN(Cc2cc(-c3ccc4c(c3)CN(C3CCC(=O)NC3=O)C4=O)nc3c2ccn3C)CC1]

The title compound was prepared in a manner analogous to Example 1 by reductive amination between 4-methylpiperidine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calcd. for C₂₈H₃₁N₅O₃, 485.24; m/z found, 486.25 [M+H]⁺. ¹H NMR (400 MHZ, DMSO-d₆) δ 11.02 (s, 1H), 8.38 (s, 1H), 8.33 (d, J=7.8 Hz, 1H), 7.85 (d, J=7.2 Hz, 1H), 7.73 (s, 1H), 7.56 (s, 1H), 6.67 (s, 1H), 5.18-5.14 (m, 1H), 4.58 (d, J=17.2 Hz, 1H), 4.44 (d, J=17.2 Hz, 1H), 3.91 (s, 3H), 3.81 (s, 2H), 3.02-2.75 (m, 3H), 2.65-2.58 (m, 1H), 2.45-2.42 (m, 1H), 2.06-2.03 (m, 3H), 1.57 (s, 2H), 1.57-1.11 (m, 3H), 0.90 (d, J=6.4 Hz, 3H).

### Example 82:3-(6-fluoro-5-(1-methyl-4-(pyrrolidin-1-ylmethyl)-1H-pyrrolo[2,3-b]pyridin-6-yl)-1-oxoisoindolin-2-yl) piperidine-2,6-dione

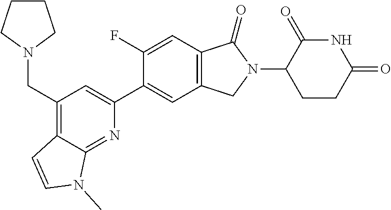 [SMILES: Cn1ccc2c(CN3CCCC3)cc(-c3cc4c(cc3F)C(=O)N(C3CCC(=O)NC3=O)C4)nc21]

The title compound was prepared in a manner analogous to Example 1 by reductive amination between pyrrolidine and 6-chloro-1-methyl-4-(pyrrolidin-1-ylmethyl)-1H-pyrrolo[2,3-b]pyridine (Intermediate 8) followed by Suzuki coupling with 3-(6-fluoro-1-oxo-5-(4,4,5,5-tetramethyl-1,3,2-dioxaborolan-2-yl) isoindolin-2-yl) piperidine-2,6-dione (Intermediate 15) as a white solid. LC-MS (ESI): mass calcd. for C₂₆H₂₆FN₅O₃, 475.20; m/z found, 476.2 [M+H]⁺. ¹H NMR (400 MHZ, DMSO) δ 11.03 (s, 1H), 8.21 (d, J=6.6 Hz, 1H), 7.66 (d, J=10.0 Hz, 1H), 7.60 (d, J=3.4 Hz, 2H), 6.65 (d, J=3.4 Hz, 1H), 5.16 (dd, J=13.3, 5.0 Hz, 1H), 4.56 (d, J=17.2 Hz, 1H), 4.42 (d, J=17.2 Hz, 1H), 3.96 (s, 2H), 3.88 (s, 3H), 3.03-2.87 (m, 1H), 2.65-2.62 (m, 1H), 2.55 (s, 4H), 2.46-2.36 (m, 1H), 2.03-1.96 (m, 1H), 1.73 (m, 4H). ¹⁹F NMR (376 MHZ, DMSO) δ-117.08 (s).

### Example 83:3-(6-chloro-5-(1-methyl-4-(pyrrolidin-1-ylmethyl)-1H-pyrrolo[2,3-b]pyridin-6-yl)-1-oxoisoindolin-2-yl) piperidine-2,6-dione

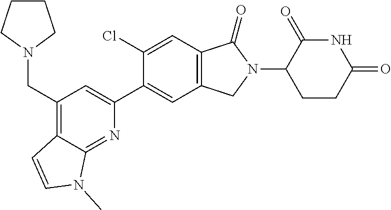 [SMILES: Cn1ccc2c(CN3CCCC3)cc(-c3cc4c(cc3Cl)C(=O)N(C3CCC(=O)NC3=O)C4)nc21]

The title compound was prepared in a manner analogous to Example 1 by Suzuki coupling 6-chloro-1-methyl-4-(pyrrolidin-1-ylmethyl)-1H-pyrrolo[2,3-b]pyridine (Intermediate 8) with 3-(6-chloro-1-oxo-5-(4,4,5,5-tetramethyl-1,3,2-dioxaborolan-2-yl) isoindolin-2-yl) piperidine-2,6-dione (Intermediate 34) as a white solid. LC-MS (ESI): mass calcd. for C₂₆H₂₆ClNO₃, 491.17; m/z found, 493.0 [M+H]⁺. ¹H NMR (400 MHZ, DMSO-d₆) δ 11.03 (s, 1H), 7.88-7.87 (m, 2H), 7.58 (d, J=3.4 Hz, 1H), 7.38 (s, 1H), 6.65 (d, J=3.4 Hz, 1H), 5.17-5.14 (m, 1H), 4.56 (d, J=17.6 Hz, 1H), 4.42 (d, J=17.6 Hz, 1H), 3.94 (s, 2H), 3.84 (s, 3H), 2.94-2.88 (m, 1H), 2.65-2.58 (m, 1H), 2.53 (s, 4H), 2.47-2.38 (m, 1H), 2.10-2.06 (m, 1H), 1.74-1.71 (m, 4H).

### Example 84:3-(5-(4-(azetidin-1-ylmethyl)-1H-pyrrolo[2,3-b]pyridin-6-yl)-1-oxoisoindolin-2-yl) piperidine-2,6-dione

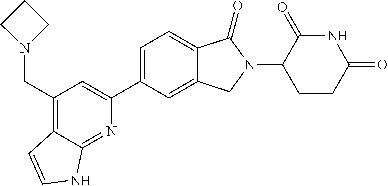 [SMILES: O=C1CCC(N2Cc3cc(-c4cc(CN5CCC5)c5cc[nH]c5n4)ccc3C2=O)C(=O)N1]

The title compound was prepared in a manner analogous to Example 1 by reductive amination between azetidine and 6-chloro-1H-pyrrolo[2,3-b]pyridine-4-carbaldehyde (Intermediate 9) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₄H₂₃N₅O₃, 429.18; m/z found, 430.2 [M+H]⁺. ¹H NMR (400 MHZ, DMSO-d₆) δ 11.76 (s, 1H), 11.02 (s, 1H), 8.31 (s, 2H), 8.25 (d, J=8.0 Hz, 1H), 7.83 (d, J=8.0 Hz, 1H), 7.67 (s, 1H), 7.52-7.50 (m, 1H), 6.61-6.59 (m, 1H), 5.18-5.13 (m, 1H), 4.57 (d, J=17.2 Hz, 1H), 4.44 (d, J=17.2 Hz, 1H), 3.90 (s, 2H), 3.26 (t, J=7.0 Hz, 4H), 2.99-2.89 (m, 1H), 2.68-2.60 (m, 1H), 2.46-2.38 (m, 1H), 2.08-2.01 (m, 3H).

### Example 85:3-(5-(4-((3-methoxyazetidin-1-yl)methyl)-1-methyl-1H-pyrrolo[2,3-b]pyridin-6-yl)-1-oxoisoindolin-2-yl) piperidine-2,6-dione

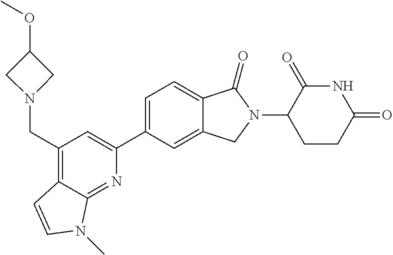 [SMILES: COC1CN(Cc2cc(-c3ccc4c(c3)CN(C3CCC(=O)NC3=O)C4=O)nc3c2ccn3C)C1]

The title compound was prepared in a manner analogous to Example 1 by reductive amination between 3-methoxyazetidine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₆H₂₇N₅O₄, 473.2; m/z found, 474.2 [M+H]⁺. ¹H NMR (400 MHZ, DMSO-d₆) δ 11.02 (s, 1H), 8.31 (s, 1H), 8.25 (d, J=9.2 Hz, 1H), 7.83 (d, J=8.0 Hz, 1H), 7.67 (s, 1H), 7.56-7.47 (m, 1H), 6.60 (d, J=3.4 Hz, 1H), 5.19-5.14 (m, 1H), 4.58 (d, J=17.2 Hz, 1H), 4.45 (d, J=17.2 Hz, 1H), 4.05-4.03 (m, 1H), 3.98 (s, 2H), 3.90 (s, 3H), 3.63-3.59 (m, 2H), 3.16 (s, 3H), 2.99-2.90 (m, 3H), 2.67-2.60 (m, 1H), 2.47-2.40 (m, 1H), 2.08-2.04 (m, 1H).

### Example 86:3-(5-(1-methyl-4-((6-oxohexahydropyrrolo[1,2-a]pyrazin-2 (1H)-yl)methyl)-1H-pyrrolo[2,3-b]pyridin-6-yl)-1-oxoisoindolin-2-yl) piperidine-2,6-dione

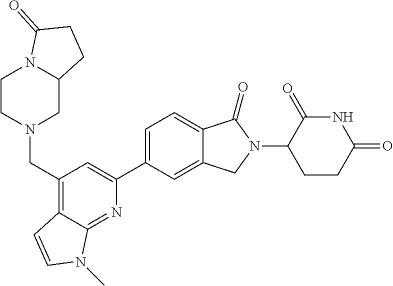 [SMILES: Cn1ccc2c(CN3CCN4C(=O)CCC4C3)cc(-c3ccc4c(c3)CN(C3CCC(=O)NC3=O)C4=O)nc21]

The title compound was prepared in a manner analogous to Example 1 by reductive amination between hexahydropyrrolo[1,2-a]pyrazin-6 (2H)-one and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₉H₃₀N₆O₄, 526.2; m/z found, 527.2 [M+H]⁺. ¹H NMR (400 MHz, DMSO-d₆) δ 11.02 (s, 1H), 8.38 (s, 1H), 8.35 (d, J=8.0 Hz, 1H), 7.85 (d, J=8.0 Hz, 1H), 7.77 (s, 1H), 7.58 (d, J=3.4 Hz, 1H), 6.70 (d, J=3.4 Hz, 1H), 5.19-5.14 (m, 1H), 4.58 (d, J=17.2 Hz, 1H), 4.45 (d, J=17.2 Hz, 1H), 3.96-3.86 (m, 5H), 3.83-3.75 (m, 1H), 3.65-3.58 (m, 1H), 3.05-2.77 (m, 4H), 2.69-2.59 (m, 1H), 2.47-2.40 (m, 1H), 2.30-2.15 (m, 2H), 2.12-1.93 (m, 3H), 1.79 (t, J=10.8 Hz, 1H), 1.52-1.44 (m, 1H).

### Example 87:3-(5-(1-methyl-4-(1-(pyrrolidin-1-yl)ethyl)-1H-pyrrolo[2,3-b]pyridin-6-yl)-1-oxoisoindolin-2-yl) piperidine-2,6-dione

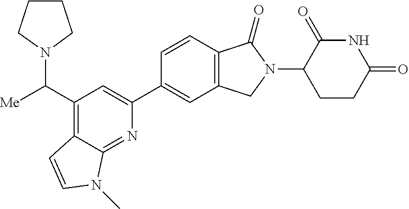 [SMILES: CC(c1cc(-c2ccc3c(c2)CN(C2CCC(=O)NC2=O)C3=O)nc2c1ccn2C)N1CCCC1]

The title compound was prepared in a manner analogous to Example 1 by Suzuki coupling 6-chloro-1-methyl-4-(1-(pyrrolidin-1-yl)ethyl)-1H-pyrrolo[2,3-b]pyridine (Intermediate 37) with 3-(1-oxo-5-(4,4,5,5-tetramethyl-1,3,2-dioxaborolan-2-yl) isoindolin-2-yl) piperidine-2,6-dione (Intermediate 13) as an off-white solid. LC-MS (ESI): mass calcd. for C₂₇H₂₉N₅O, 471.23; m/z found, 472.3 [M+H]⁺. ¹H NMR (400 MHZ, DMSO-d₆) 11.03 (s, 1H), 8.34 (s, 2H), 7.99 (s, 1H), 7.92-7.83 (m, 2H), 7.58 (d, J=3.4 Hz, 1H), 7.37 (s, 1H), 6.65 (d, J=3.4 Hz, 1H), 5.20-5.15 (m, 1H), 4.59 (d, J=17.2 Hz, 1H), 4.46 (d, J=17.2 Hz, 1H), 3.86 (s, 3H), 3.57-3.55 (m, 1H), 2.99-2.89 (m, 1H), 2.67-2.56 (m, 3H), 2.47-2.42 (m, 3H), 2.08-2.02 (m, 1H), 1.70 (s, 4H), 1.44 (d, J=6.6 Hz, 3H).).

### Example 88:3-(5-(4-((1H-imidazol-1-yl)methyl)-1-methyl-1H-pyrrolo[2,3-b]pyridin-6-yl)-1-oxoisoindolin-2-yl) piperidine-2,6-dione

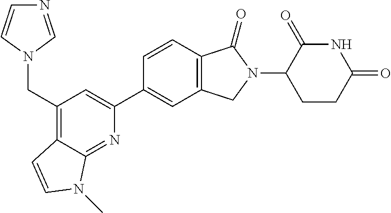 [SMILES: Cn1ccc2c(Cn3ccnc3)cc(-c3ccc4c(c3)CN(C3CCC(=O)NC3=O)C4=O)nc21]

### Step A: 4-((1H-imidazol-1-yl)methyl)-6-chloro-1-methyl-1H-pyrrolo[2,3-b]pyridine

To a solution of 4-(bromomethyl)-6-chloro-1-methyl-1H-pyrrolo[2,3-b]pyridine (Intermediate 38, 50 mg, 0.19 mmol, 1.0 eq) and imidazole (30 mg, 0.58 mmol, 3.0 eq) in DMF (2.0 mL) were K₂CO₃ (80 mg, 0.58 mmol, 3.0 eq) and 18-Crown-6 (10 mg). The mixture was stirred at 50° C. for 2 h. After cooled to room temperature, the mixture was quenched with water (20 mL) and extracted with EA (20 mL×3). The organic layer was washed with brine (20 mL×4), dried over anhydrous Na₂SO₄, filtered and concentrated under reduced pressure. The residue was purified by Prep-TLC(DCM/MeOH=8/1 v/v) to give 4-((1H-imidazol-1-yl)methyl)-6-chloro-1-methyl-1H-pyrrolo[2,3-b]pyridine (35 mg, yield 60%) as a yellow solid. LC-MS (ESI): mass calcd. for C₁₂H₁₁ClN₄, 246.07; m/z found, 246.9 [M+H]⁺.

### Step B: 3-(5-(4-((1H-imidazol-1-yl)methyl)-1-methyl-1H-pyrrolo[2,3-b]pyridin-6-yl)-1-oxoisoindolin-2-yl) piperidine-2,6-dione

To a solution of 4-((1H-imidazol-1-yl)methyl)-6-chloro-1-methyl-1H-pyrrolo[2,3-b]pyridine (405.0 mg, 162 μmol, 1.0 eq) in 1,4-Dioxane (4.00 mL) and Water (0.40 mL) were added 3-(1-oxo-5-(4,4,5,5-tetramethyl-1,3,2-dioxaborolan-2-yl) isoindolin-2-yl) piperidine-2,6-dione (Intermediate 13, 78 mg, 211 μmol, 1.3 eq) and Potassium phosphate tribasic (103 mg, 486 μmol, 3.0 eq), and 1,1′-Bis(di-t-butylphosphino) ferrocene palladium dichloride (10.6 mg, 16.2 μmol, 0.1 eq). The mixture was stirred under N₂ at 95° C. for 1.5 h. After cooled to room temperature, the reaction mixture was filtered and the filtrate was concentrated under reduced pressure. The residue was purified by Prep-TLC(DCM/MeOH=7/1 v/v) to give 3-(5-(4-((1H-imidazol-1-yl)methyl)-1-methyl-1H-pyrrolo[2,3-b]pyridin-6-yl)-1-oxoisoindolin-2-yl) piperidine-2,6-dione (6.2 mg, yield 8%) as a yellow solid. LC-MS (ESI): mass calcd. for C₂₅H₂₂N₆O₃, 454.18; m/z found, 455.3 [M+H]⁺. ¹H NMR (400 MHZ, DMSO-d₆) δ 11.02 (s, 1H), 8.35 (s, 1H), 8.29 (d, J=8.0 Hz, 1H), 7.90 (s, 1H), 7.86 (d, J=8.2 Hz, 1H), 7.69 (s, 1H), 7.61 (d, J=3.4 Hz, 1H), 7.30 (s, 1H), 6.93 (s, 1H), 6.47 (d, J=3.4 Hz, 1H), 5.58 (s, 2H), 5.17-5.13 (m, 1H), 4.57 (d, J=17.2 Hz, 1H), 4.45 (d, J=17.2 Hz, 1H), 3.91 (s, 3H), 2.99-2.89 (m, 1H), 2.65-2.60 (m, 1H), 2.48-2.38 (m, 1H), 2.08-2.02 (m, 1H).

### Example 89:3-(5-(1-methyl-4-(pyrrolidine-1-carbonyl)-1H-pyrrolo[2,3-b]pyridin-6-yl)-1-oxoisoindolin-2-yl) piperidine-2,6-dione

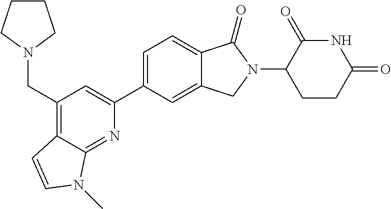 [SMILES: Cn1ccc2c(CN3CCCC3)cc(-c3ccc4c(c3)CN(C3CCC(=O)NC3=O)C4=O)nc21]

The title compound was prepared in a manner analogous to Example 1 by Suzuki coupling (6-chloro-1-methyl-1H-pyrrolo[2,3-b]pyridin-4-yl) (pyrrolidin-1-yl) methanone (Intermediate 39) with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₆H₂₅N₅O₄, 471.19; m/z found, 472.4 [M+H]⁺. ¹H NMR (400 MHZ, DMSO-d₆) δ 11.02 (s, 1H), 8.44 (s, 1H), 8.40 (d, J=8.0 Hz, 1H), 7.84 (d, J=8.8 Hz, 2H), 7.70 (d, J=3.4 Hz, 1H), 6.47 (d, J=3.4 Hz, 1H), 5.19-5.14 (m, 1H), 4.57 (d, J=17.2 Hz, 1H), 4.44 (d, J=17.2 Hz, 1H), 3.95 (s, 3H), 3.59 (t, J=6.8 Hz, 2H), 3.30 (d, J=6.8 Hz, 2H), 3.00-2.89 (m, 1H), 2.65-2.60 (m, 1H), 2.49-2.38 (m, 1H), 2.09-2.01 (m, 1H), 1.98-1.88 (m, 2H), 1.87-1.77 (m, 2H).

### Example 90:3-(5-(1-methyl-4-((4-(trifluoromethyl) piperidin-1-yl)methyl)-1H-pyrrolo[2,3-b]pyridin-6-yl)-1-oxoisoindolin-2-yl) piperidine-2,6-dione

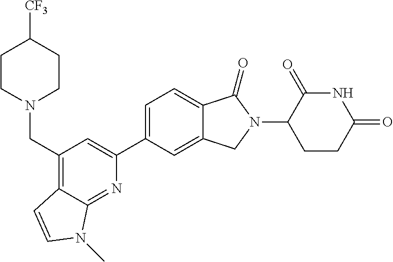 [SMILES: Cn1ccc2c(CN3CCC(C(F)(F)F)CC3)cc(-c3ccc4c(c3)CN(C3CCC(=O)NC3=O)C4=O)nc21]

The title compound was prepared in a manner analogous to Example 1 by reductive amination between 4-(trifluoromethyl) piperidine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₈H₂₈F₃N₅O₃, 539.21; m/z found, 540.3 [M+H]⁺.

¹H NMR (400 MHZ, DMSO-d₆) δ 11.02 (s, 1H), 8.38 (s, 1H), 8.34 (d, J=8.2 Hz, 1H), 7.85 (d, J=8.2 Hz, 1H), 7.74 (s, 1H), 7.57 (d, J=3.4 Hz, 1H), 6.68 (d, J=3.4 Hz, 1H), 5.18-5.14 (m, 1H), 4.58 (d, J=17.2 Hz, 1H), 4.45 (d, J=17.2 Hz, 1H), 3.91 (s, 3H), 3.87 (s, 2H), 2.99-2.91 (m, 3H), 2.65-2.59 (m, 1H), 2.47-2.38 (m, 1H), 2.36-2.31 (m, 1H), 2.07 (t, J=11.2 Hz, 3H), 1.79 (d, J=11.6 Hz, 2H), 1.51 (q, J=12.2 Hz, 2H).

### Example 91:3-(5-(4-((2-oxa-6-azaspiro[3.3]heptan-6-yl)methyl)-1-methyl-1H-pyrrolo[2,3-b]pyridin-6-yl)-1-oxoisoindolin-2-yl) piperidine-2,6-dione

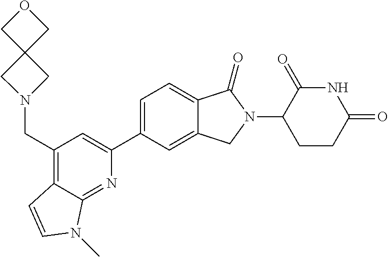 [SMILES: Cn1ccc2c(CN3CC4(COC4)C3)cc(-c3ccc4c(c3)CN(C3CCC(=O)NC3=O)C4=O)nc21]

The title compound was prepared in a manner analogous to Example 1 by reductive amination between 2-oxa-6-azaspiro[3.3]heptane and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calcd. for C₂₇H₂₇N₅O₄, 485.2; m/z found, 486.2 [M+H]⁺. ¹H NMR (400 MHZ, DMSO-d₆) δ 11.02 (s, 1H), 8.36 (s, 1H), 8.32 (d, J=8.0 Hz, 1H), 7.83 (d, J=8.0 Hz, 1H), 7.67 (s, 1H), 7.55 (d, J=3.4 Hz, 1H), 6.58 (d, J=3.4 Hz, 1H), 5.19-5.15 (m, 1H), 4.62 (s, 4H), 4.57 (d, J=17.2 Hz, 1H), 4.44 (d, J=17.2 Hz, 1H), 3.89 (s, 3H), 3.87 (s, 2H), 3.41 (s, 4H), 2.96-2.87 (m, 1H), 2.65-2.60 (m, 1H), 2.46-2.41 (m, 1H), 2.06-2.03 (m, 1H).

### Example 92:1-((6-(2-(2,6-dioxopiperidin-3-yl)-1-oxoisoindolin-5-yl)-1-methyl-1H-pyrrolo[2,3-b]pyridin-4-yl)methyl)-N,N-dimethylpiperidine-4-carboxamide

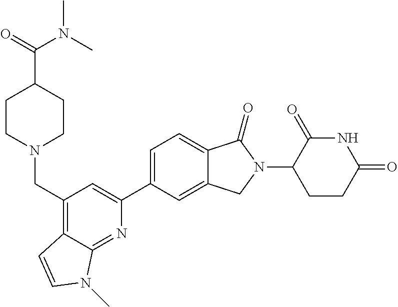 [SMILES: CN(C)C(=O)C1CCN(Cc2cc(-c3ccc4c(c3)CN(C3CCC(=O)NC3=O)C4=O)nc3c2ccn3C)CC1]

The title compound was prepared in a manner analogous to Example 1 by reductive amination between N,N-dimethylpiperidine-4-carboxamide hydrochloride and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₃₀H₃₄N₆O₄, 542.26; m/z found, 543.4 [M+H]⁺. ¹H NMR (400 MHZ, DMSO-d₆) δ 11.02 (s, 1H), 8.38 (s, 1H), 8.34 (d, J=8.0 Hz, 1H), 8.15 (s, 1H), 7.85 (d, J=8.0 Hz, 1H), 7.75 (s, 1H), 7.57 (d, J=3.4 Hz, 1H), 6.69 (d, J=3.4 Hz, 1H), 5.19-5.14 (m, 1H), 4.58 (d, J=17.2 Hz, 1H), 4.45 (d, J=17.2 Hz, 1H), 3.91 (s, 3H), 3.88 (s, 2H), 2.99 (s, 3H), 2.97-2.90 (m, 3H), 2.80 (s, 3H), 2.65-2.58 (m, 2H), 2.47-2.38 (m, 1H), 2.16-2.08 (m, 3H), 1.65-1.62 (m, 4H).

### Example 93:3-(5-(4-((3-methoxypyrrolidin-1-yl)methyl)-1-methyl-1H-pyrrolo[2,3-b]pyridin-6-yl)-1-oxoisoindolin-2-yl) piperidine-2,6-dione

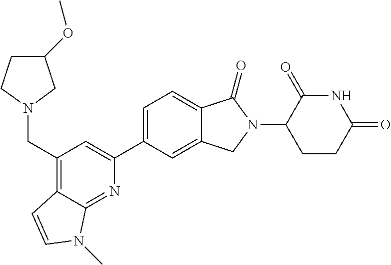 [SMILES: COC1CCN(Cc2cc(-c3ccc4c(c3)CN(C3CCC(=O)NC3=O)C4=O)nc3c2ccn3C)C1]

The title compound was prepared in a manner analogous to Example 1 by reductive amination between 3-methoxypyrrolidine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calcd. for C₂₇H₂₉N₅O₄, 487.2; m/z found, 488.3 [M+H]⁺.

¹H NMR (400 MHZ, DMSO-d₆) δ 11.01 (s, 1H), 8.36 (s, 1H), 8.33 (d, J=8.0 Hz, 1H), 7.84 (d, J=8.0 Hz, 1H), 7.75 (s, 1H), 7.56 (d, J=3.4 Hz, 1H), 6.64 (d, J=3.4 Hz, 1H), 5.18-5.13 (m, 1H), 4.57 (d, J=17.2 Hz, 1H), 4.44 (d, J=17.2 Hz, 1H), 3.96 (s, 2H), 3.90 (s, 4H), 3.15 (s, 3H), 2.95-2.86 (m, 1H), 2.65-2.55 (m, 5H), 2.45-2.36 (m, 1H), 2.08-1.97 (m, 2H), 1.70 (s, 1H).

### Example 94:3-(5-(4-((4-(1H-pyrazol-1-yl) piperidin-1-yl)methyl)-1-methyl-1H-pyrrolo[2,3-b]pyridin-6-yl)-1-oxoisoindolin-2-yl) piperidine-2,6-dione

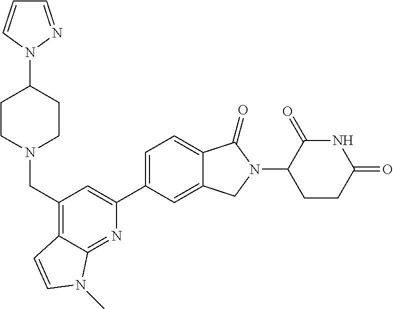 [SMILES: Cn1ccc2c(CN3CCC(n4cccn4)CC3)cc(-c3ccc4c(c3)CN(C3CCC(=O)NC3=O)C4=O)nc21]

The title compound was prepared in a manner analogous to Example 1 by reductive amination between 4-(1H-pyrazol-1-yl) piperidine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₃₀H₃₁N₇O₃, 537.25; m/z found, 538.4 [M+H]⁺. ¹H NMR (400 MHZ, DMSO-d₆) δ 11.02 (s, 1H), 8.39 (s, 1H), 8.35 (d, J=8.0 Hz, 1H), 8.15 (s, 1H), 7.85 (d, J=8.0 Hz, 1H), 7.78 (d, J=2.2 Hz, 2H), 7.58 (d, J=3.4 Hz, 1H), 7.43 (d, J=1.6 Hz, 1H), 6.71 (d, J=3.4 Hz, 1H), 6.22 (t, J=2.0 Hz, 1H), 5.19-5.14 (m, 1H), 4.58 (d, J=17.2 Hz, 1H), 4.45 (d, J=17.2 Hz, 1H), 4.22-4.13 (m, 1H), 3.92 (s, 3H), 3.91 (s, 2H), 3.02-2.90 (m, 3H), 2.65-2.58 (m, 1H), 2.47-2.40 (m, 1H), 2.24 (t, J=2.2 Hz, 2H), 2.10-2.00 (m, 5H).

### Example 95:3-(5-(4-((hexahydropyrrolo[1,2-a]pyrazin-2 (1H)-yl)methyl)-1-methyl-1H-pyrrolo[2,3-b]pyridin-6-yl)-1-oxoisoindolin-2-yl) piperidine-2,6-dione

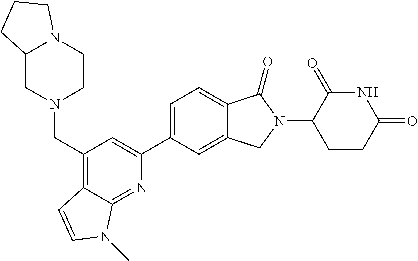 [SMILES: Cn1ccc2c(CN3CCN4CCCC4C3)cc(-c3ccc4c(c3)CN(C3CCC(=O)NC3=O)C4=O)nc21]

The title compound was prepared in a manner analogous to Example 1 by reductive amination between octahydropyrrolo[1,2-a]pyrazine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₉H₃₂N₆O₃, 512.25; m/z found, 513.4 [M+H]⁺. ¹H NMR (400 MHZ, DMSO-d₆) δ 11.02 (s, 1H), 8.37 (s, 1H), 8.33 (d, J=8.0 Hz, 1H), 8.17 (s, 1H), 7.85 (d, J=8.0 Hz, 1H), 7.74 (s, 1H), 7.56 (d, J=3.4 Hz, 1H), 6.68 (d, J=3.4 Hz, 1H), 5.19-5.14 (m, 1H), 4.58 (d, J=17.2 Hz, 1H), 4.44 (d, J=17.2 Hz, 1H), 3.93-3.87 (m, 5H), 2.98-2.92 (m, 4H), 2.79 (t, J=9.8 Hz, 1H), 2.65-2.60 (m, 1H), 2.47-2.43 (m, 1H), 2.25-2.23 (m, 2H), 2.10-2.06 (m, 3H), 1.91 (t, J=9.8 Hz, 1H), 1.71-1.63 (m, 3H), 1.31-1.21 (m, 1H).

### Example 96:3-(5-(4-((3-(hydroxymethyl) pyrrolidin-1-yl)methyl)-1-methyl-1H-pyrrolo[2,3-b]pyridin-6-yl)-1-oxoisoindolin-2-yl) piperidine-2,6-dione

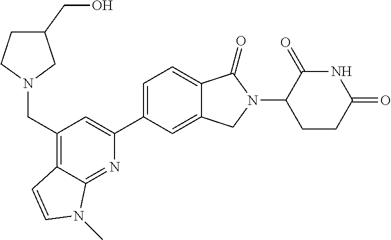 [SMILES: Cn1ccc2c(CN3CCC(CO)C3)cc(-c3ccc4c(c3)CN(C3CCC(=O)NC3=O)C4=O)nc21]

The title compound was prepared in a manner analogous to Example 1 by reductive amination between pyrrolidin-3-ylmethanol and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₇H₂₉N₅O₄, 487.22; m/z found, 488.4 [M+H]⁺. ¹H NMR (400 MHZ, DMSO-d₆) δ 11.03 (s, 1H), 10.25-10.09 (m, 1H), 8.40 (s, 1H), 8.35 (d, J=8.0 Hz, 1H), 8.04 (d, J=6.8 Hz, 1H), 7.91 (d, J=8.0 Hz, 1H), 7.75 (d, J=3.4 Hz, 1H), 6.85 (dd, J=4.8, 3.6 Hz, 1H), 5.19-5.14 (m, 1H), 4.92 (s, 1H), 4.77 (d, J=5.4 Hz, 2H), 4.60 (d, J=17.4 Hz, 1H), 4.47 (d, J=17.4 Hz, 1H), 3.95 (s, 3H), 3.48-3.24 (m, 5H), 3.05-2.89 (m, 2H), 2.64 (d, J=15.8 Hz, 1H), 2.50-2.39 (m, 2H), 2.17-2.03 (m, 2H), 1.88-1.65 (m, 1H).

### Example 97:3-(5-(4-((1H-benzo[d]imidazol-1-yl)methyl)-1-methyl-1H-pyrrolo[2,3-b]pyridin-6-yl)-1-oxoisoindolin-2-yl) piperidine-2,6-dione

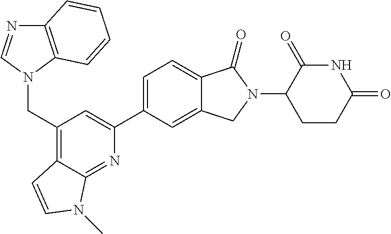 [SMILES: Cn1ccc2c(Cn3cnc4ccccc43)cc(-c3ccc4c(c3)CN(C3CCC(=O)NC3=O)C4=O)nc21]

The title compound was prepared in a manner analogous to Example 88 by displacement between 1H-benzo[d]imidazole and 4-(bromomethyl)-6-chloro-1-methyl-1H-pyrrolo[2,3-b]pyridine (Intermediate 38) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₉H₂₄N₆O₃, 504.19; m/z found, 505.5 [M+H]⁺. ¹H NMR (400 MHZ, DMSO-d₆) δ 11.02 (s, 1H), 8.58 (s, 1H), 8.34 (s, 1H), 8.29 (d, J=8.2 Hz, 1H), 7.84 (d, J=9.6 Hz, 2H), 7.69-7.64 (m, 1H), 7.58 (d, J=3.4 Hz, 1H), 7.52 (dt, J=7.2, 3.4 Hz, 1H), 7.19-7.15 (m, 2H), 6.42 (d, J=3.4 Hz, 1H), 5.89 (s, 2H), 5.18-5.13 (m, 1H), 4.57 (d, J=17.2 Hz, 1H), 4.43 (d, J=17.2 Hz, 1H), 3.88 (s, 3H), 2.99-2.89 (m, 1H), 2.67-2.59 (m, 1H), 2.46-2.41 (m, 1H), 2.08-2.01 (m, 1H).

### Example 98:3-(5-(4-(azetidin-1-ylmethyl)-1-ethyl-1H-pyrrolo[2,3-b]pyridin-6-yl)-1-oxoisoindolin-2-yl) piperidine-2,6-dione

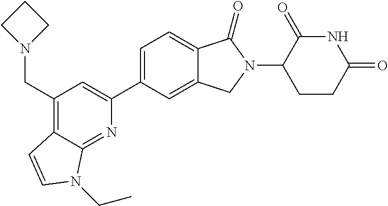 [SMILES: CCn1ccc2c(CN3CCC3)cc(-c3ccc4c(c3)CN(C3CCC(=O)NC3=O)C4=O)nc21]

The title compound was prepared in a manner analogous to Example 1 by reductive amination between azetidine and 6-chloro-1-ethyl-1H-pyrrolo[2,3-b]pyridine-4-carbaldehyde (Intermediate 10) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₆H₂₇N₅O₃, 457.21; m/z found, 458.2 [M+H]⁺. ¹H NMR (400 MHZ, DMSO-d₆) δ 11.02 (s, 1H), 8.36 (s, 1H), 8.32 (d, J=8.0 Hz, 1H), 7.85 (d, J=8.0 Hz, 1H), 7.74 (s, 1H), 7.65 (d, J=3.4 Hz, 1H), 6.65 (d, J=3.4 Hz, 1H), 5.19-5.14 (m, 1H), 4.58 (d, J=17.2 Hz, 1H), 4.45 (d, J=17.2 Hz, 1H), 4.39 (q, J=7.2 Hz, 2H), 4.03 (s, 2H), 3.39 (s, 4H), 3.00-2.90 (m, 1H), 2.66-2.61 (m, 1H), 2.46-2.41 (m, 1H), 2.14-2.03 (m, 3H), 1.45 (t, J=7.2 Hz, 3H).

### Example 99:3-(5-(1-methyl-4-((4-(1-methyl-1H-imidazol-2-yl) piperazin-1-yl)methyl)-1H-pyrrolo[2,3-b]pyridin-6-yl)-1-oxoisoindolin-2-yl) piperidine-2,6-dione

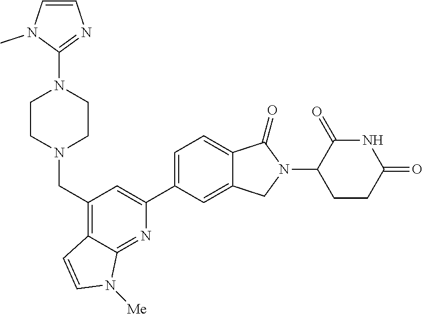 [SMILES: Cn1ccnc1N1CCN(Cc2cc(-c3ccc4c(c3)CN(C3CCC(=O)NC3=O)C4=O)nc3c2ccn3C)CC1]

The title compound was prepared in a manner analogous to Example 1 by reductive amination between 1-(1-methyl-1H-imidazol-2-yl) piperazine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₃₀H₃₂N₈O₃, 552.64; m/z found, 553.3 [M+H]⁺. ¹H NMR (400 MHZ, DMSO-d₆) δ 11.02 (s, 1H), 8.39 (s, 1H), 8.36 (d, J=8.0 Hz, 1H), 8.16 (s, 1H), 7.85 (d, J=8.0 Hz, 1H), 7.78 (s, 1H), 7.58 (d, J=3.4 Hz, 1H), 6.85 (d, J=1.4 Hz, 1H), 6.72 (d, J=3.4 Hz, 1H), 6.58 (d, J=1.0 Hz, 1H), 5.18-5.13 (m, 1H), 4.58 (d, J=17.2 Hz, 1H), 4.45 (d, J=17.2 Hz, 1H), 3.93 (s, 2H), 3.92 (s, 3H), 3.42 (s, 3H), 2.99 (d, J=4.4 Hz, 4H), 2.96-2.90 (m, 1H), 2.65-2.62 (m, 5H), 2.48-2.39 (m, 1H), 2.12-2.00 (m, 1H).

### Example 100:3-(5-(4-((7-oxa-2-azaspiro[3.5]nonan-2-yl)methyl)-1-methyl-1H-pyrrolo[2,3-b]pyridin-6-yl)-1-oxoisoindolin-2-yl) piperidine-2,6-dione

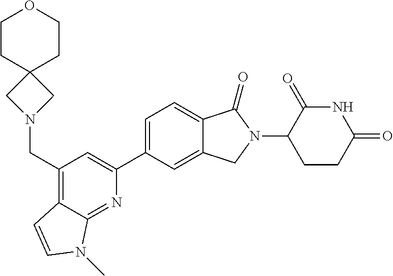 [SMILES: Cn1ccc2c(CN3CC4(CCOCC4)C3)cc(-c3ccc4c(c3)CN(C3CCC(=O)NC3=O)C4=O)nc21]

The title compound was prepared in a manner analogous to Example 1 by reductive amination between 7-oxa-2-azaspiro[3.5]nonane and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₉H₃₁N₅O₄, 513.24; m/z found, 514.3 [M+H]⁺. ¹H NMR (400 MHZ, DMSO-d₆) δ 11.01 (s, 1H), 8.37 (s, 1H), 8.33 (d, J=8.0 Hz, 1H), 8.15 (s, 1H), 7.85 (d, J=8.0 Hz, 1H), 7.71 (s, 1H), 7.57 (d, J=3.4 Hz, 1H), 6.65 (d, J=3.4 Hz, 1H), 5.19-5.14 (m, 1H), 4.58 (d, J=17.2 Hz, 1H), 4.45 (d, J=17.2 Hz, 1H), 4.01 (s, 2H), 3.91 (s, 3H), 3.50-3.46 (m, 4H), 3.14 (s, 4H), 3.00-2.90 (m, 1H), 2.66-2.61 (m, 1H), 2.47-2.43 (m, 1H), 2.07-2.04 (m, 1H), 1.71-1.68 (m, 4H).

### Example 101:3-(5-(4-((2-(hydroxymethyl) pyrrolidin-1-yl)methyl)-1-methyl-1H-pyrrolo[2,3-b]pyridin-6-yl)-1-oxoisoindolin-2-yl) piperidine-2,6-dione

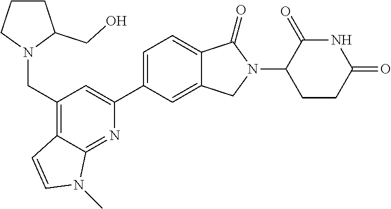 [SMILES: Cn1ccc2c(CN3CCCC3CO)cc(-c3ccc4c(c3)CN(C3CCC(=O)NC3=O)C4=O)nc21]

The title compound was prepared in a manner analogous to Example 1 by reductive amination between pyrrolidin-2-ylmethanol and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₇H₂₉N₅O₄ 487.22; m/z found, 488.4 [M+H]⁺. ¹H NMR (400 MHZ, DMSO-d₆) δ 11.01 (s, 1H), 8.38 (s, 1H), 8.33 (d, J=8.0 Hz, 1H), 7.84 (d, J=8.0 Hz, 1H), 7.77 (s, 1H), 7.54 (d, J=3.4 Hz, 1H), 6.66 (d, J=3.4 Hz, 1H), 5.17-5.13 (m, 1H), 4.57 (d, J=17.2 Hz, 1H), 4.47-4.38 (m, 2H), 3.90 (s, 3H), 3.72 (d, J=13.8 Hz, 1H), 3.56-3.52 (m, 1H), 3.41-3.39 (m, 1H), 3.38-3.37 (m, 1H), 2.99-2.88 (m, 1H), 2.84 (s, 1H), 2.65-2.58 (m, 2H), 2.46-2.37 (m, 1H), 2.30-2.19 (m, 1H), 2.09-2.01 (m, 1H), 1.93-1.81 (m, 1H), 1.69-1.53 (m, 3H).

### Example 102:3-(5-(4-((4-methoxypiperidin-1-yl)methyl)-1-methyl-1H-pyrrolo[2,3-b]pyridin-6-yl)-1-oxoisoindolin-2-yl) piperidine-2,6-dione

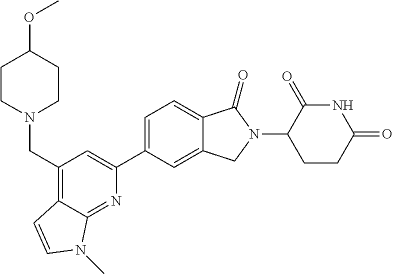 [SMILES: COC1CCN(Cc2cc(-c3ccc4c(c3)CN(C3CCC(=O)NC3=O)C4=O)nc3c2ccn3C)CC1]

The title compound was prepared in a manner analogous to Example 1 by reductive amination between 4-methoxypiperidine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₈H₃₁N₅O₄ 501.24; m/z found, 502.4 [M+H]⁺. ¹H NMR (400 MHZ, DMSO-d₆) δ 11.02 (s, 1H), 8.37 (s, 1H), 8.34 (d, J=8.0 Hz, 1H), 8.16 (s, 1H), 7.84 (d, J=8.0 Hz, 1H), 7.74 (s, 1H), 7.56 (d, J=3.4 Hz, 1H), 6.67 (d, J=3.4 Hz, 1H), 5.19-5.14 (m, 1H), 4.58 (d, J=17.2 Hz, 1H), 4.45 (d, J=17.2 Hz, 1H), 3.91 (s, 3H), 3.83 (s, 2H), 3.22 (s, 3H), 3.21-3.18 (m, 1H), 3.00-2.88 (m, 1H), 2.79-2.69 (m, 2H), 2.65-2.60 (m, 1H), 2.47-2.38 (m, 1H), 2.20 (t, J=9.6 Hz, 2H), 2.09-2.01 (m, 1H), 1.84 (d, J=9.8 Hz, 2H), 1.49-1.46 (m, 2H).

### Example 103:3-(5-(4-((8-azaspiro[4.5]decan-8-yl)methyl)-1-methyl-1H-pyrrolo[2,3-b]pyridin-6-yl)-1-oxoisoindolin-2-yl) piperidine-2,6-dio

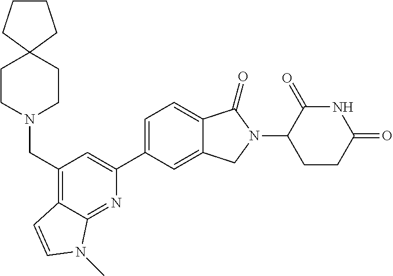 [SMILES: Cn1ccc2c(CN3CCC4(CCCC4)CC3)cc(-c3ccc4c(c3)CN(C3CCC(=O)NC3=O)C4=O)nc21]

The title compound was prepared in a manner analogous to Example 1 by reductive amination between 8-azaspiro[4.5]decane and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calcd. for C₃₁H₃₅N₅O₃, 525.27; m/z found, 526.5 [M+H]⁺. ¹H NMR (400 MHZ, DMSO-d₆) δ 11.02 (s, 1H), 8.38 (s, 1H), 8.33 (d, J=8.0 Hz, 1H), 8.15 (s, 1H), 7.87-7.80 (m, 2H), 7.58 (d, J=3.4 Hz, 1H), 6.71 (d, J=3.4 Hz, 1H), 5.18-5.13 (m, 1H), 4.58 (d, J=17.2 Hz, 1H), 4.44 (d, J=17.2 Hz, 1H), 3.98 (s, 2H), 3.91 (s, 3H), 2.99-2.88 (m, 1H), 2.65-2.57 (m, 5H), 2.47-2.38 (m, 1H), 2.09-2.01 (m, 1H), 1.56 (t, J=6.8 Hz, 4H), 1.49 (t, J=4.8 Hz, 4H), 1.39 (t, J=6.8 Hz, 4H).

### Example 104:3-(1-oxo-5-(4-(pyrrolidin-1-ylmethyl)-1,5-naphthyridin-2-yl) isoindolin-2-yl) piperidine-2,6-dione

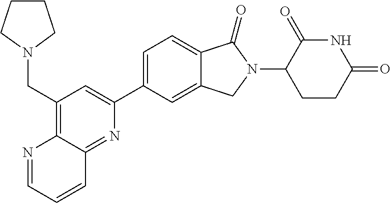 [SMILES: O=C1CCC(N2Cc3cc(-c4cc(CN5CCCC5)c5ncccc5n4)ccc3C2=O)C(=O)N1]

The title compound was prepared in a manner analogous to Example 1 by reductive amination between pyrrolidine and 2-chloro-1,5-naphthyridine-4-carbaldehyde (Intermediate 119) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₆H₂₅Ns03, 455.52; m/z found, 456.3 [M+H]⁺. ¹H NMR (400 MHZ, DMSO-d₆) δ 11.09 (s, 1H), 9.13 (d, J=3.4 Hz, 1H), 8.71 (s, 1H), 8.63 (d, J=8.2 Hz, 1H), 8.59 (s, 1H), 8.51 (d, J=8.0 Hz, 1H), 8.20 (s, 1H), 8.01 (d, J=8.0 Hz, 1H), 7.97 (dd, J=8.4, 4.2 Hz, 1H), 5.27-5.21 (m, 1H), 4.82 (s, 2H), 4.68 (d, J=17.4 Hz, 1H), 4.56 (d, J=17.4 Hz, 1H), 3.12 (s, 4H), 3.04-2.96 (m, 1H), 2.72-2.67 (m, 1H), 2.51-2.49 (m, 1H), 2.17-2.09 (m, 1H), 1.97 (s, 4H).

### Example 105:3-(1-oxo-5-(4-(pyrrolidin-1-ylmethyl)-1H-pyrazolo[3,4-b]pyridin-6-yl) isoindolin-2-yl) piperidine-2,6-dione

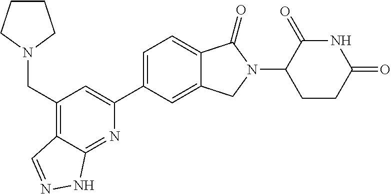 [SMILES: O=C1CCC(N2Cc3cc(-c4cc(CN5CCCC5)c5cn[nH]c5n4)ccc3C2=O)C(=O)N1]

### Step A: tert-butyl 6-chloro-4-(pyrrolidin-1-ylmethyl)-1H-pyrazolo[3,4-b]pyridine-1-carboxylate

A solution of 6-chloro-4-(pyrrolidin-1-ylmethyl)-1H-pyrazolo[3,4-b]pyridine (Intermediate 36, 50.0 mg, 211 μmol, 1.0 eq) and TEA (64.1 mg, 88.3 μL, 634 μmol, 3.0 eq) in DCM (2.0 mL) was stirred at room temperature for 10 min. Then Boc20 (69.2 mg, 72.8 μL, 317 μmol, 1.5 eq) was added to above mixture and the mixture was stirred at room temperature for 2 h. The mixture was poured into water (6 mL) and extracted with DCM (10 mL×3). The organic layer was dried over anhydrous MgSO₄, filtered and concentrated under reduced pressure. The residue was purified by Prep-TLC(DCM/MeOH=10/1 v/v) to give tert-butyl 6-chloro-4-(pyrrolidin-1-ylmethyl)-1H-pyrazolo[3,4-b]pyridine-1-carboxylate (50.0 mg, yield 63%) as a yellow oil. LC-MS (ESI): mass calcd. for C₁₆H₂₁ClN₄O₂, 336.1; m/z found, 337.1 [M+H]⁺.

### Step B: tert-butyl 6-(2-(2,6-dioxopiperidin-3-yl)-1-oxoisoindolin-5-yl)-4-(pyrrolidin-1-ylmethyl)-1H-pyrazolo[3,4-b]pyridine-1-carboxylate

To a solution of tert-butyl 6-chloro-4-(pyrrolidin-1-ylmethyl)-1H-pyrazolo[3,4-b]pyridine-1-carboxylate (50.0 mg, 148 μmol, 1.0 eq) and 3-(1-oxo-5-(4,4,5,5-tetramethyl-1,3,2-dioxaborolan-2-yl) isoindolin-2-yl) piperidine-2,6-dione (Intermediate 13, 65.9 mg, 178 μmol, 1.2 eq) in dioxane (2 mL) and water (0.2 mL) were added 1,1′-Bis(di-t-butylphosphino) ferrocene palladium dichloride (9.68 mg, 14.8 μmol, 0.1 eq) and Potassium phosphate tribasic (94.5 mg, 445 μmol, 3.0 eq). The mixture was stirred under N₂ at 95° C. for 2 h. The reaction mixture was cooled to room temperature and concentrated under reduced pressure. The residue was poured into water (5 mL) and extracted with EtOAc (10 mL×3). The organic layer was dried over anhydrous MgSO₄, filtered and concentrated under reduced pressure. The residue was purified by Prep-TLC(DCM/MeOH=10/1 v/v) to obtain tert-butyl 6-(2-(2,6-dioxopiperidin-3-yl)-1-oxoisoindolin-5-yl)-4-(pyrrolidin-1-ylmethyl)-1H-pyrazolo[3,4-b]pyridine-1-carboxylate (20.0 mg, yield 22%) as a yellow oil. LC-MS (ESI): mass calcd. for C₂₉H₃₂N₆O₅, 544.2; m/z found, 545.2 [M+H]⁺.

### Step C: 3-(1-oxo-5-(4-(pyrrolidin-1-ylmethyl)-1H-pyrazolo[3,4-b]pyridin-6-yl) isoindolin-2-yl) piperidine-2,6-dione

To a solution of tert-butyl 6-(2-(2,6-dioxopiperidin-3-yl)-1-oxoisoindolin-5-yl)-4-(pyrrolidin-1-ylmethyl)-1H-pyrazolo[3,4-b]pyridine-1-carboxylate (30.0 mg, 55.1 μmol, 1.0 eq) in dioxane (2 mL) were added HCl-dioxane (4 N) (0.2 mL, 0.2 mmol, 3.6 eq). The mixture was stirred at 25° C. for 2 h. After evaporation, the crude product was purified by Prep-HPLC with YMC-Actus Triart C18 (5 μm, 21.2×250 mm), and mobile phase of 5-95% ACN in water (0.1% FA) over 20 min and then hold at 95% ACN for 3 min, at a flow rate of 20 mL/min to obtain 3-(1-oxo-5-(4-(pyrrolidin-1-ylmethyl)-1H-pyrazolo[3,4-b]pyridin-6-yl) isoindolin-2-yl) piperidine-2,6-dione formate (2.60 mg, yield 10%) as a white solid. LC-MS (ESI): mass calcd. for C₂₄H₂₄N₆O₃, 444.2; m/z found, 445.2 [M+H]⁺. ¹H NMR (400 MHZ, DMSO-d₆) δ 13.74 (s, 1H), 11.02 (s, 1H), 8.38 (s, 1H), 8.31 (d, J=9.8 Hz, 2H), 8.15 (s, 1H), 7.87 (d, J=8.0 Hz, 2H), 5.18-5.13 (m, 1H), 4.58 (d, J=17.4 Hz, 1H), 4.45 (d, J=17.4 Hz, 1H), 4.14 (s, 2H), 2.96-2.88 (m, 1H), 2.64-2.60 (m, 5H), 2.45-2.42 (m, 1H), 2.10-2.00 (m, 1H), 1.79 (s, 4H).

### Example 106:3-(5-(4-((2-(methoxymethyl) pyrrolidin-1-yl)methyl)-1-methyl-1H-pyrrolo[2,3-b]pyridin-6-yl)-1-oxoisoindolin-2-yl) piperidine-2,6-dione

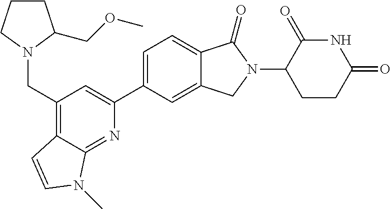 [SMILES: COCC1CCCN1Cc1cc(-c2ccc3c(c2)CN(C2CCC(=O)NC2=O)C3=O)nc2c1ccn2C]

The title compound was prepared in a manner analogous to Example 1 by reductive amination between 2-(methoxymethyl) pyrrolidine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₈H₃₁N₅O₄ 501.24; m/z found, 502.4 [M+H]⁺. ¹H NMR (400 MHZ, DMSO-d₆) δ 11.03 (s, 1H), 9.83 (s, 1H), 8.42 (s, 1H), 8.36 (d, J=8.6 Hz, 1H), 8.04 (s, 1H), 7.91 (d, J=8.0 Hz, 1H), 7.77 (d, J=3.4 Hz, 1H), 6.78 (d, J=3.4 Hz, 1H), 5.20-5.15 (m, 1H), 4.89 (d, J=12.8 Hz, 1H), 4.69-4.62 (m, 1H), 4.60 (d, J=17.4 Hz, 1H), 4.47 (d, J=17.4 Hz, 1H), 3.96 (s, 3H), 3.91 (d, J=5.0 Hz, 1H), 3.65 (d, J=6.8 Hz, 2H), 3.42-3.31 (m, 5H), 3.00-2.89 (m, 1H), 2.66-2.60 (m, 1H), 2.48-2.44 (m, 1H), 2.29-2.16 (m, 1H), 2.11-2.01 (m, 2H), 1.90-1.68 (m, 2H).

### Example 107:3-(5-(4-((6,7-dihydropyrazolo[1,5-a]pyrazin-5 (4H)-yl)methyl)-1-methyl-1H-pyrrolo[2,3-b]pyridin-6-yl)-1-oxoisoindolin-2-yl) piperidine-2,6-dione

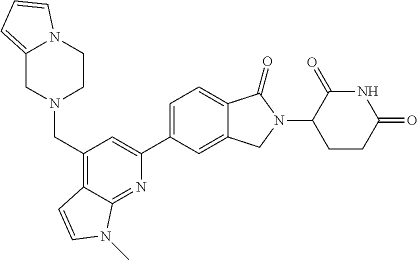 [SMILES: Cn1ccc2c(CN3CCn4cccc4C3)cc(-c3ccc4c(c3)CN(C3CCC(=O)NC3=O)C4=O)nc21]

The title compound was prepared in a manner analogous to Example 1 by reductive amination between 4,5,6,7-tetrahydropyrazolo[1,5-a]pyrazine hydrochloride and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₈H₂₇N₇O₃ 509.22; m/z found, 510.4 [M+H]⁺. ¹H NMR (400 MHZ, DMSO-d₆) δ 11.02 (s, 1H), 8.39-8.33 (m, 2H), 8.22 (s, 1H), 7.84 (d, J=8.6 Hz, 2H), 7.58 (d, J=3.4 Hz, 1H), 7.38 (d, J=1.8 Hz, 1H), 6.68 (d, J=3.4 Hz, 1H), 6.00 (d, J=1.8 Hz, 1H), 5.18-5.13 (m, 1H), 4.57 (d, J=17.4 Hz, 1H), 4.44 (d, J=17.4 Hz, 1H), 4.13-4.09 (m, 4H), 3.92 (s, 3H), 3.74 (s, 2H), 3.05-2.97 (m, 2H), 2.96-2.88 (m, 1H), 2.65-2.60 (m, 1H), 2.46-2.37 (m, 1H), 2.10-2.01 (m, 1H).

### Example 108:3-(5-(4-(((2R,5R)-2,5-dimethylpyrrolidin-1-yl)methyl)-1-methyl-1H-pyrrolo[2,3-b]pyridin-6-yl)-1-oxoisoindolin-2-yl) piperidine-2,6-dione

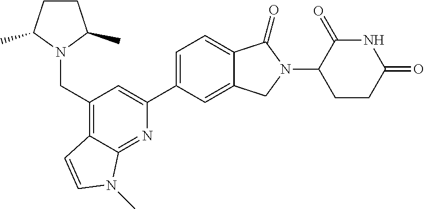 [SMILES: C[C@@H]1CC[C@@H](C)N1Cc1cc(-c2ccc3c(c2)CN(C2CCC(=O)NC2=O)C3=O)nc2c1ccn2C]

The title compound was prepared in a manner analogous to Example 1 by reductive amination between (2R,5R)-2,5-dimethylpyrrolidine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₈H₃₁N₅O₃, 485.24; m/z found, 486.4 [M+H]⁺. ¹H NMR (400 MHZ, DMSO-d₆) δ 11.08 (s, 1H), 8.48 (s, 1H), 8.42 (d, J=8.0 Hz, 1H), 8.20 (s, 1H), 8.13 (s, 1H), 7.92 (d, J=8.0 Hz, 1H), 7.68 (d, J=2.8 Hz, 1H), 6.80 (d, J=3.3 Hz, 1H), 5.25-5.20 (m, 1H), 4.64 (d, J=17.2 Hz, 1H), 4.51 (d, J=17.2 Hz, 1H), 4.44 (s, 1H), 4.29 (s, 1H), 3.98 (s, 3H), 3.05-2.95 (m, 1H), 2.69 (d, J=17.4 Hz, 1H), 2.54-2.45 (m, 1H), 2.19 (s, 2H), 2.14-2.07 (m, 1H), 1.59 (s, 2H), 1.29 (s, 1H), 1.18 (d, J=4.8 Hz, 7H).

### Example 109:3-(5-(4-((3-(methoxymethyl) pyrrolidin-1-yl)methyl)-1-methyl-1H-pyrrolo[2,3-b]pyridin-6-yl)-1-oxoisoindolin-2-yl) piperidine-2,6-dione

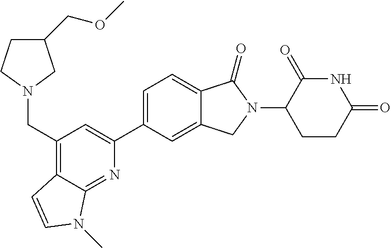 [SMILES: COCC1CCN(Cc2cc(-c3ccc4c(c3)CN(C3CCC(=O)NC3=O)C4=O)nc3c2ccn3C)C1]

The title compound was prepared in a manner analogous to Example 1 by reductive amination between 3-(methoxymethyl) pyrrolidine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₈H₃₁N₅O₄, 501.24; m/z found, 502.2 [M+H]⁺. ¹H NMR (400 MHZ, DMSO-d₆) δ 11.02 (s, 1H), 8.38 (s, 1H), 8.34 (d, J=8.2 Hz, 1H), 8.15 (s, 1H), 7.85 (d, J=8.0 Hz, 1H), 7.77 (s, 1H), 7.57 (d, J=3.4 Hz, 1H), 6.65 (d, J=3.4 Hz, 1H), 5.19-5.14 (m, 1H), 4.57 (d, J=17.2 Hz, 1H), 4.45 (d, J=17.2 Hz, 1H), 4.03-3.94 (m, 2H), 3.91 (s, 3H), 3.25 (d, J=6.8 Hz, 2H), 3.22 (s, 3H), 2.99-2.90 (m, 1H), 2.70-2.58 (m, 4H), 2.48-2.36 (m, 3H), 2.10-2.01 (m, 1H), 1.90-1.86 (m, 1H), 1.46-1.38 (m, 1H).

### Example 110:3-(5-(7-(azetidin-1-ylmethyl)-1-methyl-1H-pyrrolo[3,2-b]pyridin-5-yl)-1-oxoisoindolin-2-yl) piperidine-2,6-dione

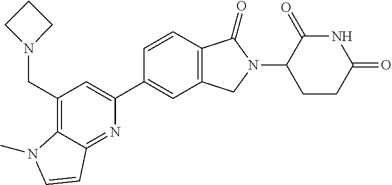 [SMILES: Cn1ccc2nc(-c3ccc4c(c3)CN(C3CCC(=O)NC3=O)C4=O)cc(CN3CCC3)c21]

The title compound was prepared in a manner analogous to Example 1 by reductive amination between azetidine and 5-chloro-1-methyl-1H-pyrrolo[3,2-b]pyridine-7-carbaldehyde (Intermediate 12) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for C₂₅H₂₅N₅O₃ 443; m/z found, 444.2 [M+H]⁺. ¹H NMR (400 MHZ, DMSO-d₆) δ 11.02 (s, 1H), 8.35 (s, 1H), 8.27 (d, J=8.0 Hz, 1H), 7.82 (d, J=8.0 Hz, 1H), 7.71 (s, 1H), 7.60 (d, J=3.2 Hz, 1H), 6.61 (d, J=3.2 Hz, 1H), 5.18-5.13 (m, 1H), 4.56 (d, J=17.2 Hz, 1H), 4.43 (d, J=17.2 Hz, 1H), 4.12 (s, 5H), 3.22 (s, 4H), 2.94-2.89 (m, 1H), 2.65-2.60 (m, 1H), 2.48-2.40 (m, 1H), 2.08-2.03 (m, 3H).

### Example 111:3-(5-(4-((3-hydroxyazetidin-1-yl)methyl)-1H-pyrrolo[2,3-b]pyridin-6-yl)-1-oxoisoindolin-2-yl) piperidine-2,6-dione

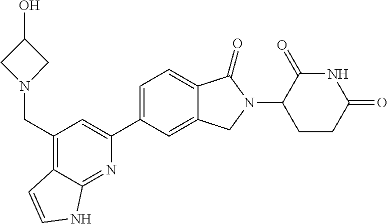 [SMILES: O=C1CCC(N2Cc3cc(-c4cc(CN5CC(O)C5)c5cc[nH]c5n4)ccc3C2=O)C(=O)N1]

The title compound was prepared in a manner analogous to Example 1 by reductive amination between azetidin-3-ol and 6-chloro-1-methyl-1H-pyrrolo[2,3-b]pyridine-4-carbaldehyde (Intermediate 9) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a gray solid. LC-MS (ESI): mass calcd. for C₂₄H₂₃N₅O₄, 445.2; m/z found, 446.2 [M+H]⁺. 1H NMR (400 MHZ, DMSO-d₆) δ 11.81 (s, 1H), 11.02 (s, 1H), 8.31 (s, 1H), 8.26 (d, J=8.0 Hz, 1H), 8.15 (s, 1H), 7.84 (d, J=8.0 Hz, 1H), 7.72 (s, 1H), 7.55 (s, 1H), 6.63 (s, 1H), 5.51 (s, 1H), 5.17-5.13 (m, 1H), 4.57 (d, J=17.2 Hz, 1H), 4.44 (d, J=17.2 Hz, 1H), 4.32 (s, 1H), 4.10 (s, 2H), 3.75 (s, 2H), 3.10 (s, 2H), 3.00-2.88 (m, 1H), 2.65-2.61 (m, 1H), 2.48-2.36 (m, 1H), 2.09-2.01 (m, 1H).

### Example 112:3-(5-(4-((2-azaspiro[3.3]heptan-2-yl)methyl)-1-methyl-1H-pyrrolo[2,3-b]pyridin-6-yl)-1-oxoisoindolin-2-yl) piperidine-2,6-dione

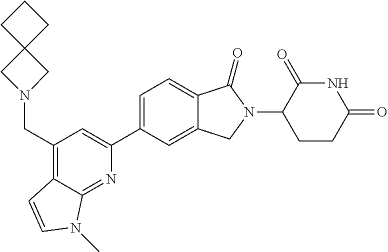 [SMILES: Cn1ccc2c(CN3CC4(CCC4)C3)cc(-c3ccc4c(c3)CN(C3CCC(=O)NC3=O)C4=O)nc21]

The title compound was prepared in a manner analogous to Example 1 by reductive amination between 2-azaspiro[3.3]heptane and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₈H₂₉N₅O₃, 483.2; m/z found, 484.3 [M+H]⁺. ¹H NMR (400 MHZ, DMSO-d₆) δ 11.03 (s, 1H), 8.38 (s, 1H), 8.33 (d, J=8.2 Hz, 1H), 8.15 (s, 1H), 7.86 (d, J=8.0 Hz, 1H), 7.73 (s, 1H), 7.60 (d, J=3.4 Hz, 1H), 6.65 (d, J=3.4 Hz, 1H), 5.19-5.14 (m, 1H), 4.58 (d, J=17.2 Hz, 1H), 4.45 (d, J=17.2 Hz, 1H), 4.05 (s, 2H), 3.91 (s, 3H), 3.42 (s, 4H), 3.01-2.88 (m, 1H), 2.65-2.61 (m, 1H), 2.48-2.37 (m, 1H), 2.15-2.00 (m, 5H), 1.80-1.71 (m, 2H).

### Example 113:3-(5-(4-((2,2-dimethylpyrrolidin-1-yl)methyl)-1-methyl-1H-pyrrolo[2,3-b]pyridin-6-yl)-1-oxoisoindolin-2-yl) piperidine-2,6-dione

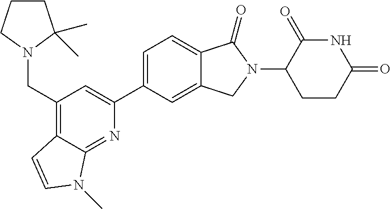 [SMILES: Cn1ccc2c(CN3CCCC3(C)C)cc(-c3ccc4c(c3)CN(C3CCC(=O)NC3=O)C4=O)nc21]

The title compound was prepared in a manner analogous to Example 1 by reductive amination between 2,2-dimethylpyrrolidine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₈H₃₁N₅O₃, 485.59; m/z found, 486.3 [M+H]⁺. ¹H NMR (400 MHZ, DMSO-d₆) δ 11.03 (s, 1H), 8.37 (s, 1H), 8.32 (d, J=8.0 Hz, 1H), 8.23 (s, 1H), 7.84 (d, J=8.0 Hz, 1H), 7.74 (s, 1H), 7.54 (d, J=3.4 Hz, 1H), 6.65 (d, J=3.4 Hz, 1H), 5.18-5.13 (m, 1H), 4.58 (d, J=17.2 Hz, 1H), 4.44 (d, J=17.2 Hz, 1H), 3.90 (s, 3H), 3.87 (s, 2H), 3.01-2.90 (m, 1H), 2.70-2.55 (m, 3H), 2.44-2.38 (m, 1H), 2.09-2.02 (m, 1H), 1.67 (s, 4H), 1.16 (s, 6H).

### Example 114:3-(5-(1-methyl-4-(pyrrolidin-1-yl)-1H-pyrrolo[2,3-b]pyridin-6-yl)-1-oxoisoindolin-2-yl) piperidine-2,6-dione

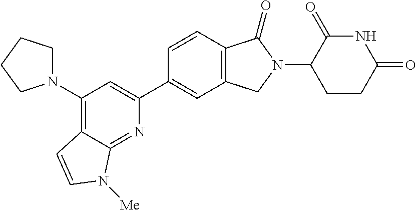 [SMILES: Cn1ccc2c(N3CCCC3)cc(-c3ccc4c(c3)CN(C3CCC(=O)NC3=O)C4=O)nc21]

The title compound was prepared in a manner analogous to Example 1 by Suzuki coupling of 6-chloro-1-methyl-4-(pyrrolidin-1-yl)-1H-pyrrolo[2,3-b]pyridine (Intermediate 40) with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calcd. for C₂₅H₂₅N₅O₃, 443.51; m/z found, 444.4 [M+H]⁺. ¹H NMR (400 MHZ, DMSO-d₆) δ 11.01 (s, 1H), 7.95 (s, 1H), 7.86 (s, 2H), 7.13 (d, J=3.4 Hz, 1H), 6.42 (s, 1H), 6.40 (d, J=3.4 Hz, 1H), 5.18-5.13 (m, 1H), 4.57 (d, J=17.4 Hz, 1H), 4.44 (d, J=17.4 Hz, 1H), 3.72 (s, 3H), 3.52 (s, 4H), 2.98-2.89 (m, 1H), 2.67-2.60 (m, 1H), 2.46-2.32 (m, 1H), 2.08-2.02 (m, 1H), 1.97 (s, 4H).

### Example 115:3-(5-(4-((4-(1HIimidazol-1-yl) piperidin-1-yl)methyl)-1-methyl-1H-pyrrolo[2,3-b]pyridin-6-yl)-1-oxoisoindolin-2-yl) piperidine-2,6-dione

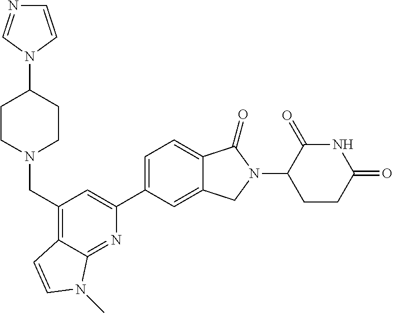 [SMILES: Cn1ccc2c(CN3CCC(n4ccnc4)CC3)cc(-c3ccc4c(c3)CN(C3CCC(=O)NC3=O)C4=O)nc21]

The title compound was prepared in a manner analogous to Example 1 by reductive amination between 4-(1H-imidazol-1-yl) piperidine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₃₀H₃₁N₇O₃, 537.62; m/z found, 538.4 [M+H]⁺. ¹H NMR (400 MHZ, DMSO-d₆) δ 11.02 (s, 1H), 8.38 (s, 1H), 8.34 (d, J=8.2 Hz, 1H), 8.14 (s, 1H), 7.96 (s, 1H), 7.85 (d, J=8.0 Hz, 1H), 7.77 (s, 1H), 7.57 (d, J=3.4 Hz, 1H), 7.44 (s, 1H), 6.99 (s, 1H), 6.71 (d, J=3.4 Hz, 1H), 5.18-5.13 (m, 1H), 4.57 (d, J=17.2 Hz, 1H), 4.45 (d, J=17.2 Hz, 1H), 4.17 (s, 1H), 3.91 (s, 5H), 3.02 (d, J=11.2 Hz, 2H), 2.98-2.88 (m, 1H), 2.65-2.60 (m, 1H), 2.48-2.38 (m, 1H), 2.21-2.18 (m, 2H), 2.06-2.02 (m, 1H), 1.97 (s, 4H).

### Example 116:3-(5-(4-((4-fluoropiperidin-1-yl)methyl)-1H-pyrrolo[2,3-b]pyridin-6-yl)-1-oxoisoindolin-2-yl) piperidine-2,6-dione

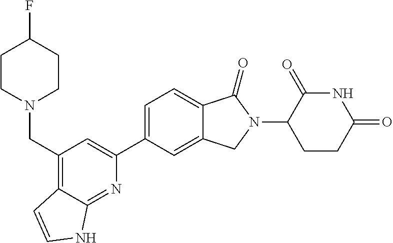 [SMILES: O=C1CCC(N2Cc3cc(-c4cc(CN5CCC(F)CC5)c5cc[nH]c5n4)ccc3C2=O)C(=O)N1]

The title compound was prepared in a manner analogous to Example 1 by reductive amination between 4-fluoropiperidine and 6-chloro-1H-pyrrolo[2,3-b]pyridine-4-carbaldehyde (Intermediate 9) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₆H₂₆FN₅O₃475.2; m/z found, 476.3 [M+H]⁺. ¹H NMR (400 MHz, DMSO-d₆) δ 11.76 (s, 1H), 11.02 (s, 1H), 8.31 (s, 1H), 8.26 (d, J=8.0 Hz, 1H), 8.14 (s, 1H), 7.83 (d, J=8.0 Hz, 1H), 7.72 (s, 1H), 7.55-7.48 (m, 1H), 6.67 (dd, J=3.4, 1.8 Hz, 1H), 5.18-5.13 (m, 1H), 4.72-4.60 (m, 1H), 4.57 (d, J=17.2 Hz, 1H), 4.44 (d, J=17.2 Hz, 1H), 3.87 (s, 2H), 3.02-2.94 (m, 1H), 2.63 (d, J=15.6 Hz, 3H), 2.49-2.39 (m, 3H), 2.10-2.00 (m, 1H), 1.89-1.82 (m, 2H), 1.78-1.76 (m, 2H).

### Example 117:3-(5-(4-((3-fluoroazetidin-1-yl)methyl)-1H-pyrrolo[2,3-b]pyridin-6-yl)-1-oxoisoindolin-2-yl) piperidine-2,6-dione

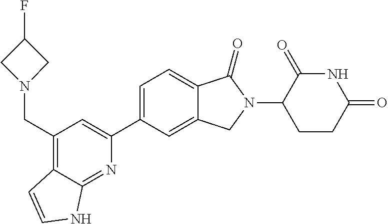 [SMILES: O=C1CCC(N2Cc3cc(-c4cc(CN5CC(F)C5)c5cc[nH]c5n4)ccc3C2=O)C(=O)N1]

The title compound was prepared in a manner analogous to Example 1 by reductive amination between 3-fluoroazetidine hydrochloride and 6-chloro-1H-pyrrolo[2,3-b]pyridine-4-carbaldehyde (Intermediate 9) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₄H₂₂FN₅O₃, 447.17; m/z found, 448.3 [M+H]⁺. ¹H NMR (400 MHZ, DMSO-d₆) δ 11.77 (s, 1H), 11.01 (s, 1H), 8.30 (s, 1H), 8.25 (d, J=8.0 Hz, 1H), 8.16 (s, 1H), 7.83 (d, J=8.0 Hz, 1H), 7.67 (s, 1H), 7.55-7.50 (m, 1H), 6.60 (dd, J=3.2, 1.8 Hz, 1H), 5.35-5.10 (m, 2H), 4.56 (d, J=17.2 Hz, 1H), 4.43 (d, J=17.2 Hz, 1H), 4.01 (s, 2H), 3.70-3.64 (m, 2H), 3.25-3.21 (m, 2H), 2.98-2.89 (m, 1H), 2.65-2.60 (m, 1H), 2.46-2.37 (m, 1H), 2.08-1.98 (m, 1H).

### Example 118:3-(5-(1-(2-methoxyethyl)-4-(pyrrolidin-1-ylmethyl)-1H-pyrrolo[2,3-b]pyridin-6-yl)-1-oxoisoindolin-2-yl) piperidine-2,6-dione

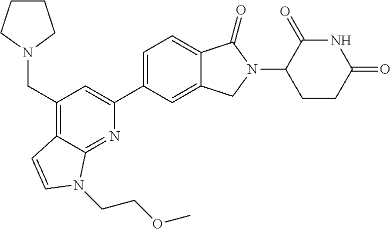 [SMILES: COCCn1ccc2c(CN3CCCC3)cc(-c3ccc4c(c3)CN(C3CCC(=O)NC3=O)C4=O)nc21]

### Step A: 6-chloro-1-(2-methoxyethyl)-1H-pyrrolo[2,3-b]pyridine-4-carbaldehyde

To a solution of 6-chloro-1H-pyrrolo[2,3-b]pyridine-4-carbaldehyde (Intermediate 9, 100 mg, 554 μmol, 1.0 eq) in anhydrous DMF (5 mL) was added Cs₂CO₃ (361 mg, 1.11 mmol, 2.0 eq) at room temperature and the reaction mixture was stirred for 15 min. Then 1-bromo-2-methoxyethane (115 mg, 831 μmol, 1.5 eq) was added to above mixture and the reaction mixture was stirred at 80° C. for 1 h. After cooled to room temperature, the reaction mixture was quenched with ice-cold water (15 mL) and extracted with ethyl acetate (30 mL×3). The combined organic layer was washed with water (30 mL×4) and brine (30 mL), dried over anhydrous Na₂SO₄, filtered and concentrated under reduced pressure. The crude product was purified by flash column chromatography on silica gel (ethyl acetate in petroleum ether, from 0% to 20% v/v) to get 6-chloro-1-(2-methoxyethyl)-1H-pyrrolo[2,3-b]pyridine-4-carbaldehyde (50.0 mg, yield 38%) as a yellow solid. LC-MS (ESI): mass calced for: C₁₁H₁₁ClN₂O₂, 238.05; m/z found, 239.1 [M+H]⁺.

### Step B: 3-(5-(1-(2-methoxyethyl)-4-(pyrrolidin-1-ylmethyl)-1H-pyrrolo[2,3-b]pyridin-6-yl)-1-oxoisoindolin-2-yl) piperidine-2,6-dione

The title compound was prepared in a manner analogous to Example 1 by reductive amination between pyrrolidine and 6-chloro-1-(2-methoxyethyl)-1H-pyrrolo[2,3-b]pyridine-4-carbaldehyde followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₈H₃₁N₅O₄, 501.24; m/z found, 502.3 [M+H]⁺. ¹H NMR (400 MHz, DMSO-d₆) δ 11.02 (s, 1H), 8.35 (s, 1H), 8.31 (d, J=8.0 Hz, 1H), 8.19 (s, 1H), 7.84 (d, J=8.0 Hz, 1H), 7.76 (s, 1H), 7.59 (d, J=3.0 Hz, 1H), 6.64 (d, J=3.0 Hz, 1H), 5.17-5.13 (m, 1H), 4.60-4.42 (m, 4H), 3.97 (s, 2H), 3.83-3.76 (m, 2H), 3.27 (s, 3H), 3.00-2.89 (m, 1H), 2.67-2.55 (m, 5H), 2.46-2.40 (m, 1H), 2.07-2.05 (m, 1H), 1.75 (s, 4H).

### Example 119:3-(5-(1-cyclopropyl-4-(pyrrolidin-1-ylmethyl)-1H-pyrrolo[2,3-b]pyridin-6-yl)-1-oxoisoindolin-2-yl) piperidine-2,6-dione

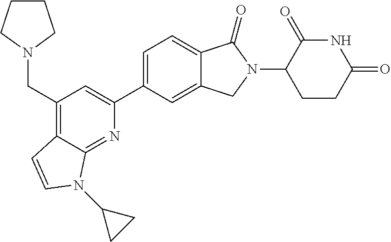 [SMILES: O=C1CCC(N2Cc3cc(-c4cc(CN5CCCC5)c5ccn(C6CC6)c5n4)ccc3C2=O)C(=O)N1]

### Step A: 6-chloro-1-(2-methoxyethyl)-1H-pyrrolo[2,3-b]pyridine-4-carbaldehyde

To a stirred solution of 6-chloro-1H-pyrrolo[2,3-b]pyridine-4-carbaldehyde (Intermediate 9, 100 mg, 554 μmol, 1.0 eq) in MeCN (5.00 mL) were added cyclopropylboronic acid (238 mg, 2.77 mmol, 5.0 eq), Cupric acetate monohydrate (221 mg, 1.11 mmol, 2.0 eq), and TEA (560 mg, 772 μL, 5.54 mmol, 10.0 eq). The reaction mixture was stirred under dry air atmosphere at room temperature for 24 h. The reaction mixture was diluted with EtOAc (30 mL) and filtered. The filtrate was concentrated under reduced pressure and the crude product was purified by flash column chromatography on silca gel (ethyl acetate in petroleum ether, from 0% to 20% v/v) to afford 6-chloro-1-cyclopropyl-1H-pyrrolo[2,3-b]pyridine-4-carbaldehyde (30.0 mg, yield 24%) as a yellow solid. LC-MS (ESI): mass calced for: C₁₁H₉ClN₂O, 220.04; m/z found, 221.3 [M+H]⁺.

### Step B: 3-(5-(1-cyclopropyl-4-(pyrrolidin-1-ylmethyl)-1H-pyrrolo[2,3-b]pyridin-6-yl)-1-oxoisoindolin-2-yl) piperidine-2,6-dione

The title compound was prepared in a manner analogous to Example 1 by reductive amination between pyrrolidine and 6-chloro-1-cyclopropyl-4-(pyrrolidin-1-ylmethyl)-1H-pyrrolo[2,3-b]pyridine followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₈H₂₉N₅O₃, 483.23; m/z found, 484.2 [M+H]⁺. ¹H NMR (400 MHZ, DMSO-d₆) δ 11.02 (s, 1H), 8.37 (s, 1H), 8.34 (d, J=8.0 Hz, 1H), 8.14 (s, 1H), 7.86 (d, J=8.0 Hz, 1H), 7.83 (s, 1H), 7.53 (d, J=3.4 Hz, 1H), 6.64 (d, J=3.4 Hz, 1H), 5.18-5.14 (m, 1H), 4.58 (d, J=17.2 Hz, 1H), 4.45 (d, J=17.2 Hz, 1H), 4.08 (s, 2H), 3.75 (s, 1H), 2.99-2.89 (m, 1H), 2.72-2.60 (m, 5H), 2.47-2.42 (m, 1H), 2.07-2.02 (m, 1H), 1.78 (s, 4H), 1.12-1.10 (m, 4H).

### Example 120:3-(5-(4-((3-hydroxypyrrolidin-1-yl)methyl)-1H-pyrrolo[2,3-b]pyridin-6-yl)-1-oxoisoindolin-2-yl) piperidine-2,6-dione

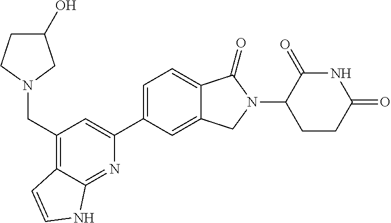 [SMILES: O=C1CCC(N2Cc3cc(-c4cc(CN5CCC(O)C5)c5cc[nH]c5n4)ccc3C2=O)C(=O)N1]

The title compound was prepared in a manner analogous to Example 1 by reductive amination between pyrrolidin-3-ol and 6-chloro-1H-pyrrolo[2,3-b]pyridine-4-carbaldehyde (Intermediate 9) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₅H₂₅N₅O₄, 459.19; m/z found, 460.3 [M+H]⁺. ¹H NMR (400 MHZ, DMSO-d₆) δ 11.79 (s, 1H), 11.01 (s, 1H), 8.31 (s, 1H), 8.25 (d, J=8.0 Hz, 1H), 8.13 (s, 1H), 7.83 (d, J=8.0 Hz, 1H), 7.78 (s, 1H), 7.56-7.51 (m, 1H), 6.66-6.64 (m, 1H), 5.18-5.13 (m, 1H), 4.83 (s, 1H), 4.56 (d, J=17.2 Hz, 1H), 4.43 (d, J=17.2 Hz, 1H), 4.27 (s, 1H), 4.07 (s, 2H), 2.94-2.89 (m, 3H), 2.69-2.54 (m, 3H), 2.48-2.37 (m, 1H), 2.07-2.03 (m, 2H), 1.64 (s, 1H).

### Example 121:3-(5-(4-((3-oxa-7-azabicyclo[3.3.1]nonan-7-yl)methyl)-1H-pyrrolo[2,3-b]pyridin-6-yl)-1-oxoisoindolin-2-yl) piperidine-2,6-dio

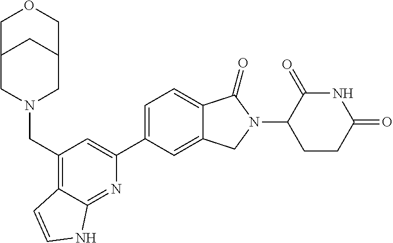 [SMILES: O=C1CCC(N2Cc3cc(-c4cc(CN5CC6COCC(C6)C5)c5cc[nH]c5n4)ccc3C2=O)C(=O)N1]

The title compound was prepared in a manner analogous to Example 1 by reductive amination between 3-oxa-7-azabicyclo[3.3.1]nonane and 6-chloro-1H-pyrrolo[2,3-b]pyridine-4-carbaldehyde (Intermediate 9) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₈H₂₉N₅O₄, 499.22; m/z found, 500.3 [M+H]⁺. ¹H NMR (400 MHZ, DMSO-d₆) δ 11.73 (s, 1H), 11.02 (s, 1H), 8.30 (s, 1H), 8.25 (d, J=8.2 Hz, 1H), 7.86-7.58 (m, 3H), 6.90-6.88 (m, 1H), 5.18-5.13 (m, 1H), 4.57 (d, J=17.2 Hz, 1H), 4.44 (d, J=17.2 Hz, 1H), 3.92 (s, 2H), 3.69 (d, J=10.8 Hz, 4H), 3.51-3.33 (m, 2H), 3.28-3.02 (m, 2H), 2.94-2.89 (m, 1H), 2.65-2.59 (m, 1H), 2.46-2.41 (m, 1H), 2.06-2.03 (m, 1H), 1.83 (s, 4H).

### Example 122:3-(6-fluoro-1-oxo-5-(4-(pyrrolidin-1-ylmethyl)-1H-pyrrolo[2,3-b]pyridin-6-yl) isoindolin-2-yl) piperidine-2,6-dione

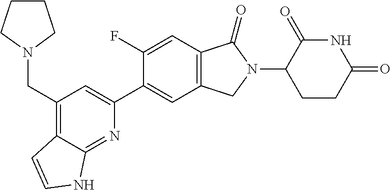 [SMILES: O=C1CCC(N2Cc3cc(-c4cc(CN5CCCC5)c5cc[nH]c5n4)c(F)cc3C2=O)C(=O)N1]

The title compound was prepared in a manner analogous to Example 1 by Suzuki coupling 6-chloro-4-(pyrrolidin-1-ylmethyl)-1H-pyrrolo[2,3-b]pyridine (Intermediate 9) with 3-(6-fluoro-1-oxo-5-(4,4,5,5-tetramethyl-1,3,2-dioxaborolan-2-yl) isoindolin-2-yl) piperidine-2,6-dione (Intermediate 15) as a light-yellow solid. LC-MS (ESI): mass calcd. for C₂₄H₂₄FN₅O₃, 461.19; m/z found, 462.2 [M+H]⁺. ¹H NMR (400 MHZ, DMSO-d₆) δ 11.93 (s, 1H), 11.03 (s, 1H), 8.15 (s, 1H), 8.13 (s, 1H), 7.66 (d, J=9.8 Hz, 2H), 7.60 (s, 1H), 6.71 (s, 1H), 5.18-5.14 (m, 1H), 4.55 (d, J=17.2 Hz, 1H), 4.42 (d, J=17.2 Hz, 1H), 4.19 (s, 2H), 3.00-2.88 (m, 1H), 2.77 (s, 2H), 2.64-2.58 (m, 1H), 2.49-2.40 (m, 3H), 2.10-2.02 (m, 1H), 1.81 (s, 4H). 1° F. NMR (376 MHz, DMSO) δ -117.07 (s).

### Example 123:3-(5-(4-((2,2-dimethylpyrrolidin-1-yl)methyl)-1-methyl-1H-pyrrolo[2,3-b]pyridin-6-yl)-1-oxoisoindolin-2-yl) piperidine-2,6-dione

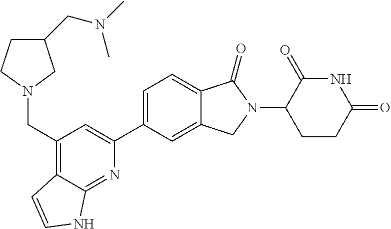 [SMILES: CN(C)CC1CCN(Cc2cc(-c3ccc4c(c3)CN(C3CCC(=O)NC3=O)C4=O)nc3[nH]ccc23)C1]

The title compound was prepared in a manner analogous to Example 1 by reductive amination between N,N-dimethyl-1-(pyrrolidin-3-yl) methanamine and 6-chloro-1H-pyrrolo[2,3-b]pyridine-4-carbaldehyde (Intermediate 9) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₈H₃₂N₆O₃, 500.6; m/z found, 501.3 [M+H]⁺. ¹H NMR (400 MHZ, DMSO-d₆) δ 11.76 (s, 1H), 11.01 (s, 1H), 8.30 (s, 1H), 8.25 (d, J=8.0 Hz, 1H), 8.17 (s, 1H), 7.83 (d, J=8.0 Hz, 1H), 7.72 (s, 1H), 7.52 (s, 1H), 6.63 (s, 1H), 5.18-5.13 (m, 1H), 4.56 (d, J=17.2 Hz, 1H), 4.43 (d, J=17.2 Hz, 1H), 3.95 (t, J=7.6 Hz, 2H), 2.95-2.89 (m, 1H), 2.70-2.60 (m, 5H), 2.45-2.35 (m, 4H), 2.26 (s, 6H), 2.05-1.94 (m, 2H), 1.50-1.39 (m, 1H).

### Example 124:3-(5-(7-(azetidin-1-ylmethyl)-1-ethyl-1H-pyrrolo[3,2-b]pyridin-5-yl)-1-oxoisoindolin-2-yl) piperidine-2,6-dione

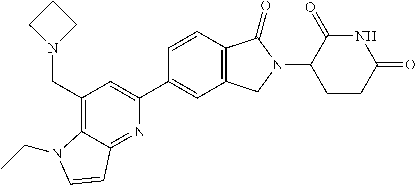 [SMILES: CCn1ccc2nc(-c3ccc4c(c3)CN(C3CCC(=O)NC3=O)C4=O)cc(CN3CCC3)c21]

The title compound was prepared in a manner analogous to Example 1 by reductive amination between azetidine and 5-chloro-1-ethyl-1H-pyrrolo[3,2-b]pyridine-7-carbaldehyde (Intermediate 41) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as an off-white solid. LC-MS (ESI): mass calcd. for C₂₆H₂₇N₅O₃, 457.21; m/z found, 458.6 [M+H]⁺. ¹H NMR (400 MHz, DMSO-d₆) δ 11.01 (s, 1H), 8.34 (s, 1H), 8.26 (d, J=8.0 Hz, 1H), 8.14 (s, 1H), 7.81 (d, J=8.0 Hz, 1H), 7.72 (s, 1H), 7.68 (d, J=3.0 Hz, 1H), 6.65 (d, J=3.0 Hz, 1H), 5.17-5.13 (m, 1H), 4.53-4.45 (m, 4H), 3.97 (s, 2H), 3.25 (t, J=6.6 Hz, 4H), 2.98-2.88 (m, 1H), 2.64-2.56 (m, 1H), 2.47-2.40 (m, 1H), 2.05-2.01 (m, 3H), 1.40 (t, J=7.1 Hz, 3H).

### Example 125:3-(5-(4-((4-hydroxypiperidin-1-yl)methyl)-1H-pyrrolo[2,3-b]pyridin-6-yl)-1-oxoisoindolin-2-yl) piperidine-2,6-dione

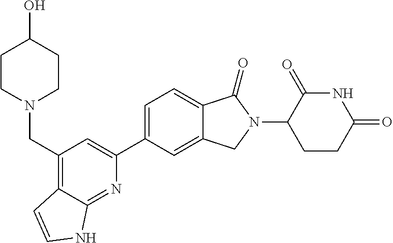 [SMILES: O=C1CCC(N2Cc3cc(-c4cc(CN5CCC(O)CC5)c5cc[nH]c5n4)ccc3C2=O)C(=O)N1]

The title compound was prepared in a manner analogous to Example 1 by reductive amination between piperidin-4-ol and 6-chloro-1H-pyrrolo[2,3-b]pyridine-4-carbaldehyde (Intermediate 9) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₆H₂₇N₅O₄, 473.21; m/z found, 474.3 [M+H]⁺. ¹H NMR (400 MHZ, DMSO-d₆) δ 11.78 (s, 1H), 11.01 (s, 1H), 8.31 (s, 1H), 8.25 (d, J=7.8 Hz, 1H), 8.14 (s, 1H), 7.84 (d, J=7.8 Hz, 1H), 7.74 (s, 1H), 7.53 (s, 1H), 6.68 (s, 1H), 5.18-5.13 (m, 1H), 4.57 (d, J=17.2 Hz, 2H), 4.44 (d, J=17.2 Hz, 1H), 3.89 (s, 2H), 3.52 (s, 1H), 2.98-2.89 (m, 1H), 2.82 (s, 2H), 2.70-2.58 (m, 1H), 2.46-2.36 (m, 1H), 2.25 (s, 2H), 2.08-2.03 (m, 1H), 1.79-1.73 (m, 2H), 1.51-1.45 (m, 2H).

### Example 126:3-(4-fluoro-1-oxo-5-(4-(pyrrolidin-1-ylmethyl)-1H-pyrrolo[2,3-b]pyridin-6-yl) isoindolin-2-yl) piperidine-2,6-dione

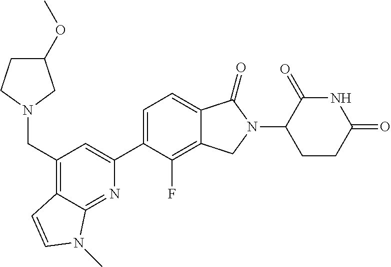 [SMILES: COC1CCN(Cc2cc(-c3ccc4c(c3F)CN(C3CCC(=O)NC3=O)C4=O)nc3c2ccn3C)C1]

The title compound was prepared in a manner analogous to Example 1 by reductive amination between pyrrolidine and 6-chloro-1-methyl-1H-pyrrolo[2,3-b]pyridine-4-carbaldehyde (Intermediate 8) followed by Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14) as a yellow solid. LC-MS (ESI): mass calcd. for C₂₅H₂₄FN₅O₃, 461.2; m/z found, 462.2 [M+H]⁺. ¹H NMR (400 MHZ, DMSO-d₆) δ 11.98 (s, 1H), 11.03 (s, 1H), 8.14 (s, 1H), 8.11 (d, J=7.2 Hz, 1H), 7.78-7.70 (m, 2H), 7.64 (s, 1H), 6.75 (s, 1H), 5.18-5.13 (m, 1H), 4.66 (d, J=17.4 Hz, 1H), 4.49 (d, J=17.4 Hz, 1H), 4.33 (s, 2H), 3.04-2.81 (m, 5H), 2.65-2.60 (m, 1H), 2.47-2.40 (m, 1H), 2.13-1.99 (m, 1H), 1.85 (s, 4H).

### Example 127:3-(5-(4-((3-fluoropyrrolidin-1-yl)methyl)-1H-pyrrolo[2,3-b]pyridin-6-yl)-1-oxoisoindolin-2-yl) piperidine-2,6-dione

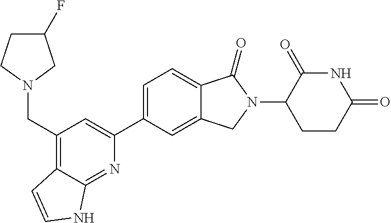 [SMILES: O=C1CCC(N2Cc3cc(-c4cc(CN5CCC(F)C5)c5cc[nH]c5n4)ccc3C2=O)C(=O)N1]

The title compound was prepared in a manner analogous to Example 1 by reductive amination between 3-fluoropyrrolidine and 6-chloro-1H-pyrrolo[2,3-b]pyridine-4-carbaldehyde (Intermediate 9) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₅H₂₄FN504, 461.19; m/z found, 462.3 [M+H]⁺. ¹H NMR (400 MHZ, DMSO-d₆) δ 11.76 (s, 1H), 11.01 (s, 1H), 8.30 (s, 1H), 8.24 (d, J=7.8 Hz, 1H), 7.82 (d, J=7.8 Hz, 1H), 7.72 (s, 1H), 7.52 (s, 1H), 6.64 (s, 1H), 5.33-5.11 (m, 2H), 4.56 (d, J=17.2 Hz, 1H), 4.43 (d, J=17.2 Hz, 1H), 3.99 (s, 2H), 2.92-2.64 (m, 4H), 2.44-2.32 (m, 3H), 2.27-2.09 (m, 1H), 2.08-2.01 (m, 1H), 2.00-1.82 (m, 1H).

### Example 128:3-(5-(1-cyclobutyl-4-(pyrrolidin-1-ylmethyl)-1H-pyrrolo[2,3-b]pyridin-6-yl)-1-oxoisoindolin-2-yl) piperidine-2,6-dione

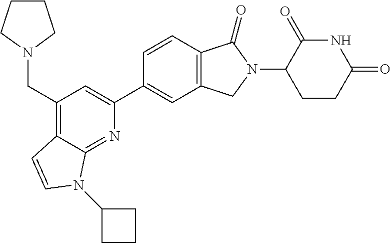 [SMILES: O=C1CCC(N2Cc3cc(-c4cc(CN5CCCC5)c5ccn(C6CCC6)c5n4)ccc3C2=O)C(=O)N1]

The title compound was prepared in a manner analogous to Example 1 by reductive amination between pyrrolidine and 6-chloro-1H-pyrrolo[2,3-b]pyridine-4-carbaldehyde (Intermediate 42) followed by Suzuki coupling with 6-chloro-1-cyclobutyl-1H-pyrrolo[2,3-b]pyridine-4-carbaldehyde (Intermediate 13) as an off-white solid. LC-MS (ESI): mass calcd. for C₂₉H₃₁N₅O₃, 497.24; m/z found, 498.5 [M+H]⁺. ¹H NMR (400 MHZ, DMSO-d₆) δ 11.01 (s, 1H), 8.36 (s, 1H), 8.32 (d, J=8.0 Hz, 1H), 8.16 (s, 1H), 7.88-7.81 (m, 2H), 7.76 (s, 1H), 6.68 (d, J=3.2 Hz, 1H), 5.49-5.40 (m, 1H), 5.18-5.13 (m, 1H), 4.57 (d, J=17.2 Hz, 1H), 4.44 (d, J=17.2 Hz, 1H), 3.97 (s, 2H), 3.00-2.88 (m, 1H), 2.98-2.53 (m, 7H), 2.49-2.31 (m, 3H), 2.09-2.01 (m, 1H), 1.95-1.84 (m, 2H), 1.74 (s, 4H).

### Example 129:3-(5-(1-(oxetan-3-yl)-4-(pyrrolidin-1-ylmethyl)-1H-pyrrolo[2,3-b]pyridin-6-yl)-1-oxoisoindolin-2-yl) piperidine-2,6-dione

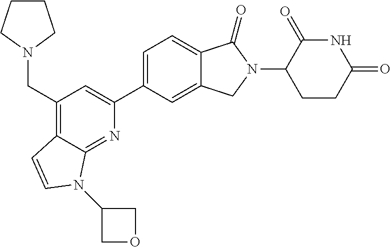 [SMILES: O=C1CCC(N2Cc3cc(-c4cc(CN5CCCC5)c5ccn(C6COC6)c5n4)ccc3C2=O)C(=O)N1]

The title compound was prepared in a manner analogous to Example 1 by reductive amination between pyrrolidine and 6-chloro-1-(oxetan-3-yl)-1H-pyrrolo[2,3-b]pyridine-4-carbaldehyde (Intermediate 29) followed by Suzuki coupling with 6-chloro-1-cyclobutyl-1H-pyrrolo[2,3-b]pyridine-4-carbaldehyde (Intermediate 13) as a white solid. LC-MS (ESI): mass calcd. for C₂₈H₂₉N₅O₄, 499.2; m/z found, 500.2 [M+H]⁺. ¹H NMR (400 MHZ, DMSO-d₆) δ 11.02 (s, 1H), 8.36 (s, 1H), 8.33 (d, J=8.0 Hz, 1H), 8.19 (s, 1H), 7.99 (d, J=3.6 Hz, 1H), 7.84 (d, J=8.0 Hz, 1H), 7.80 (s, 1H), 6.78 (d, J=3.6 Hz, 1H), 6.15-6.10 (m, 1H), 5.18-5.13 (m, 1H), 5.06 (t, J=7.0 Hz, 4H), 4.57 (d, J=17.2 Hz, 1H), 4.44 (d, J=17.2 Hz, 1H), 3.98 (s, 2H), 3.00-2.89 (m, 1H), 2.65-2.63 (m, 1H), 2.60-2.52 (m, 4H), 2.47-2.37 (m, 1H), 2.09-2.00 (m, 1H), 1.78-1.70 (m, 4H).

### Example 130:3-(5-(1-(2-(benzyloxy)ethyl)-4-(pyrrolidin-1-ylmethyl)-1H-pyrrolo[2,3-b]pyridin-6-yl)-1-oxoisoindolin-2-yl) piperidine-2,6-dione

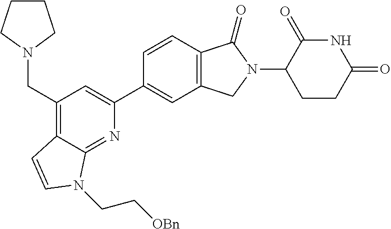 [SMILES: O=C1CCC(N2Cc3cc(-c4cc(CN5CCCC5)c5ccn(CCOCc6ccccc6)c5n4)ccc3C2=O)C(=O)N1]

### Step A: methyl 1-(2-(benzyloxy)ethyl)-6-chloro-1H-pyrrolo[2,3-b]pyridine-4-carboxylate

To a solution of methyl 6-chloro-1H-pyrrolo[2,3-b]pyridine-4-carboxylate (500 mg, 2.37 mmol, 1.0 eq) in DMF (10.0 mL) were added ((2-bromoethoxy)methyl)benzene (766 mg, 3.56 mmol, 1.5 eq) and Cs₂CO₃ (1.55 g, 4.75 mmol, 2.0 eq). The mixture was stirred under N₂ at 70° C. for 3 h. The resulting mixture was cooled to room temperature, diluted with H₂O (30 mL), and extracted with EA (40 mL×3). The organic layer was washed with brine (30 mL×4), dried over anhydrous Na₂SO₄, filtered and concentrated under reduced pressure. The residue was purified by flash column chromatography on silica gel (ethyl acetate in petroleum ether, from 0% to 40% v/v) to give methyl 1-(2-(benzyloxy)ethyl)-6-chloro-1H-pyrrolo[2,3-b]pyridine-4-carboxylate (610 mg, yield 74%) as an off-white solid. LC-MS (ESI): mass calcd. for C₁₈H₁₇ClN₂O₃, 344.09; m/z found, 345.1 [M+H]⁺.

### Step B: (1-(2-(benzyloxy)ethyl)-6-chloro-1H-pyrrolo[2,3-b]pyridin-4-yl) methanol

To a solution of methyl 1-(2-(benzyloxy)ethyl)-6-chloro-1H-pyrrolo[2,3-b]pyridine-4-carboxylate (600 mg, 1.74 mmol, 1.0 eq) in THF (10.0 mL) was added LiAlH₄ (132 mg, 3.48 mmol, 2.0 eq) in portions at 0° C. The mixture was stirred under N₂ at 0° C. for 1 h. The resulting mixture was quenched with Na₂SO₄·10H₂O and filtered. The filtrate was concentrated under reduced pressure to give (1-(2-(benzyloxy)ethyl)-6-chloro-1H-pyrrolo[2,3-b]pyridin-4-yl) methanol (400 mg, yield 73%) as a white solid. LC-MS (ESI): mass calcd. for C₁₇H₁₇ClN₂O₂, 316.10; m/z found, 317.2 [M+H]⁺.

### Step C: 1-(2-(benzyloxy)ethyl)-6-chloro-1H-pyrrolo[2,3-b]pyridine-4-carbaldehyde

To a solution of (1-(2-(benzyloxy)ethyl)-6-chloro-1H-pyrrolo[2,3-b]pyridin-4-y1) methanol (400 mg, 1.26 mmol, 1.0 eq) in DMSO (10.0 mL) was added IBX (707 mg, 2.53 mmol, 2.0 eq) and the mixture was stirred at 30° C. for 3 h. The resulting mixture was quenched with ice water (30 mL) and extracted with EtOAc (50 mL×3). The combined organic phases were washed with brine (50 mL×4), dried over anhydrous Na₂SO₄, filtered and concentrated under reduced pressure. The residue was purified by flash column chromatography on silica gel (ethyl acetate in petroleum ether, from 0% to 70% v/v) to give 1-(2-(benzyloxy)ethyl)-6-chloro-1H-pyrrolo[2,3-b]pyridine-4-carbaldehyde (210 mg, yield 53%) as a white solid. LC-MS (ESI): mass calcd. for C₁₇H₁₅ClN₂O₂, 314.08; m/z found, 315.2 [M+H]⁺.

### Step D: 3-(5-(1-(2-(benzyloxy)ethyl)-4-(pyrrolidin-1-ylmethyl)-1H-pyrrolo[2,3-b]pyridin-6-yl)-1-oxoisoindolin-2-yl) piperidine-2,6-dione

The title compound was prepared in a manner analogous to Example 1 by reductive amination between pyrrolidine and 1-(2-(benzyloxy)ethyl)-6-chloro-1H-pyrrolo[2,3-b]pyridine-4-carbaldehyde followed by Suzuki coupling with 6-chloro-1-cyclobutyl-1H-pyrrolo[2,3-b]pyridine-4-carbaldehyde (Intermediate 13) as an off-white solid. LC-MS (ESI): mass calcd. for C₃₄H₃₅N₅O₄, 577.27; m/z found, 578.7 [M+H]⁺. ¹H NMR (400 MHZ, DMSO-d6) δ 11.00 (s, 1H), 8.38-8.22 (m, 2H), 8.15 (s, 1H), 7.82 (d, J=7.8 Hz, 1H), 7.75 (s, 1H), 7.61 (s, 1H), 7.25-7.18 (m, 5H), 6.65 (s, 1H), 5.17-5.13 (m, 1H), 4.64-4.34 (m, 6H), 3.97 (s, 2H), 3.89 (s, 2H), 2.94-2.90 (m, 1H), 2.65 (s, 1H), 2.57 (s, 4H), 2.45-2.40 (m, 1H), 2.07-2.03 (m, 1H), 1.74 (s, 4H).

### Example 131:3-(5-(1-(2-hydroxyethyl)-4-(pyrrolidin-1-ylmethyl)-1H-pyrrolo[2,3-b]pyridin-6-yl)-1-oxoisoindolin-2-yl) piperidine-2,6-dione

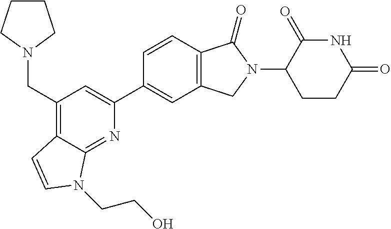 [SMILES: O=C1CCC(N2Cc3cc(-c4cc(CN5CCCC5)c5ccn(CCO)c5n4)ccc3C2=O)C(=O)N1]

To a solution of 3-(5-(1-(2-(benzyloxy)ethyl)-4-(pyrrolidin-1-ylmethyl)-1H-pyrrolo[2,3-b]pyridin-6-yl)-1-oxoisoindolin-2-yl) piperidine-2,6-dione (Example 130, 100 mg, 173 μmol, 1.0 eq) in 2,2,2-Trifluoroethanol (10.0 mL) was added 10% Pd/C(36.8 mg) at 0° C. The mixture was stirred under H₂ (1 atm) for 1 h at 0° C. The mixture was filtered and the filtrate was concentrated under reduced pressure. The residue was purified by Prep-HPLC with YMC-Actus Triart 18C(5 μm, 20×250 mm), and mobile phase of 5-99% ACN in water (0.1% FA) over 10 min and then hold at 100% ACN for 2 min, at a flow rate of 25 mL/min to afford 3-(5-(1-(2-hydroxyethyl)-4-(pyrrolidin-1-ylmethyl)-1H-pyrrolo[2,3-b]pyridin-6-yl)-1-oxoisoindolin-2-yl) piperidine-2,6-dione formate (2.00 mg, yield 2%) as a white solid. LC-MS (ESI): mass calcd. for C₂₇H₂₉N₅O₄, 487.22; m/z found, 488.3 [M+H]⁺. ¹H NMR (400 MHZ, DMSO-d₆) δ 11.01 (s, 1H), 8.34 (s, 1H), 8.31 (d, J=8.0 Hz, 1H), 8.27 (s, 1H), 7.84 (d, J=8.0 Hz, 1H), 7.74 (s, 1H), 7.58 (s, 1H), 6.62 (s, 1H), 5.18-5.13 (m, 1H), 4.97 (s, 1H), 4.57 (d, J=17.6 Hz, 1H), 4.41-4.38 (m, 3H), 3.94 (s, 2H), 3.83 (t, J=5.6 Hz, 2H), 2.94-2.90 (m, 1H), 2.67-2.54 (m, 5H), 2.47-2.40 (m, 1H), 2.07-2.00 (m, 1H), 1.74 (s, 4H).

### Example 132 3-(5-(1-(2-hydroxyethyl)-4-methyl-2,3-dihydro-1H-pyrrolo[2,3-b]pyridin-6-yl)-1-oxoisoindolin-2-yl) piperidine-2,6-dione

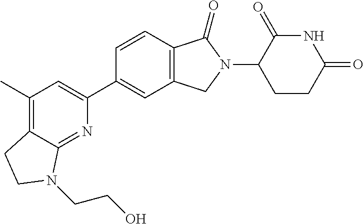 [SMILES: Cc1cc(-c2ccc3c(c2)CN(C2CCC(=O)NC2=O)C3=O)nc2c1CCN2CCO]

To a solution of 3-(5-(1-(2-(benzyloxy)ethyl)-4-(pyrrolidin-1-ylmethyl)-1H-pyrrolo[2,3-b]pyridin-6-yl)-1-oxoisoindolin-2-yl) piperidine-2,6-dione (Example 130, 0.0 mg, 156 μmol, 1.0 eq) in ethyl acetate (10.0 mL) and AcOH (10 mL) was added 10% Pd/C(50 mg). The mixture was stirred under H₂ (1 atm) at 40° C. for 10 h. The mixture was cooled to room temperature and filtered. The filtrate was concentrated under reduced pressure. The residue was purified by Prep-HPLC with YMC-Actus Triart 18C(5 μm, 20×250 mm), and mobile phase of 5-99% ACN in water (0.1% FA) over 10 min and then hold at 100% ACN for 2 min, at a flow rate of 25 mL/min to give 3-(5-(1-(2-hydroxyethyl)-4-methyl-2,3-dihydro-1H-pyrrolo[2,3-b]pyridin-6-yl)-1-oxoisoindolin-2-yl) piperidine-2,6-dione formate (2.00 mg, yield 3.5%) as a white solid. LC-MS (ESI): mass calcd. for C₂₇H₂₉N₅O₄, 420.18; m/z found, 421.2 [M+H]⁺. ¹H NMR (400 MHZ, DMSO-d₆) δ 10.99 (s, 1H), 8.19 (s, 1H), 8.14 (d, J=8.0 Hz, 1H), 7.75 (d, J=8.0 Hz, 1H), 7.01 (s, 1H), 5.15-5.10 (m, 1H), 4.78 (t, J=4.8 Hz, 1H), 4.52 (d, J=17.2 Hz, 1H), 4.38 (d, J=17.2 Hz, 1H), 3.68-3.58 (m, 4H), 3.46 (t, J=5.6 Hz, 2H), 2.97-2.88 (m, 3H), 2.67-2.58 (m, 1H), 2.45-2.36 (m, 1H), 2.18 (s, 3H), 2.05-1.99 (m, 1H).

### Example 133:2-(6-(2-(2,6-dioxopiperidin-3-yl)-1-oxoisoindolin-5-yl)-4-(pyrrolidin-1-ylmethyl)-1H-pyrrolo[2,3-b]pyridin-1-yl) acetonitrile

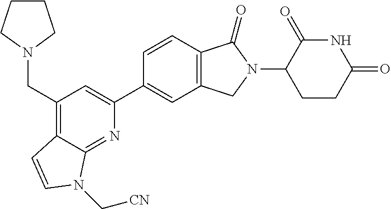 [SMILES: N#CCn1ccc2c(CN3CCCC3)cc(-c3ccc4c(c3)CN(C3CCC(=O)NC3=O)C4=O)nc21]

### Step A: methyl 6-chloro-1-(cyanomethyl)-1H-pyrrolo[2,3-b]pyridine-4-carboxylate

To a solution of methyl 6-chloro-1H-pyrrolo[2,3-b]pyridine-4-carboxylate (500 mg, 2.37 mmol, 1.0 eq) in DMF (10.0 mL) were added Cs₂CO₃ (1.55 g, 4.75 mmol, 2.0 eq) and 2-bromoacetonitrile (427 mg, 3.56 mmol, 1.5 eq). The mixture was stirred under N₂ at 70° C. for 3 h. The resulting mixture was cooled to room temperature, diluted with H₂O (30 mL), and extracted with EA (50 mL×3). The organic layer was washed with brine (20 mL×4), dried over anhydrous Na₂SO₄, filtered and concentrated under reduced pressure. The residue was purified by flash column chromatography on silica gel (ethyl acetate in petroleum ether, from 0% to 30% v/v) to give methyl 6-chloro-1-(cyanomethyl)-1H-pyrrolo[2,3-b]pyridine-4-carboxylate (420 mg, yield 70%) as an off-white solid. LC-MS (ESI): mass calcd. for C₁₁H₈ClN₃O₂, 249.03; m/z found, 250.1 [M+H]⁺.

### Step B: 2-(6-chloro-4-(hydroxymethyl)-1H-pyrrolo[2,3-b]pyridin-1-yl) acetonitrile

To a solution of methyl 6-chloro-1-(cyanomethyl)-1H-pyrrolo[2,3-b]pyridine-4-carboxylate (400 mg, 1.60 mmol, 1.0 eq) in MeOH (10.0 mL) was added NaBH₄ (300 mg, 8.00 mmol, 5.0 eq) in portions. The mixture was stirred at room temperature for 3 h. The resulting mixture was quenched with MeOH (10 mL) and concentrated under reduced pressure. The residue was purified by flash column chromatography on silica gel (ethyl acetate in petroleum ether, from 0% to 50% v/v) to give 2-(6-chloro-4-(hydroxymethyl)-1H-pyrrolo[2,3-b]pyridin-1-yl) acetonitrile (250 mg, yield 78%) as a white solid. LC-MS (ESI): mass calcd. for C₁₀H₈ClN₃O, 221.04; m/z found, 222.1 [M+H]⁺.

### Step C: 2-(6-chloro-4-formyl-1H-pyrrolo[2,3-b]pyridin-1-yl) acetonitrile

To a solution n of 2-(6-chloro-4-(hydroxymethyl)-1H-pyrrolo[2,3-b]pyridin-1-yl) acetonitrile (250 mg, 1.35 mmol, 1.0 eq) in DMSO (10.0 mL) was added IBX (682 mg, 2.44 mmol, 1.8 eq) in portions and the mixture was stirred at 30° C. for 3 h. The resulting mixture was quenched with ice-water (30 mL) and extracted with EtOAc (50 mL×3). The combined organic phases were washed with brine (50 mL×4), dried over anhydrous Na₂SO₄, filtered and concentrated under reduced pressure. The residue was purified by flash column chromatography on silica gel (ethyl acetate in petroleum ether, from 0% to 50%) to give 2-(6-chloro-4-formyl-1H-pyrrolo[2,3-b]pyridin-1-yl) acetonitrile (150 mg, yield: 70%) as a white solid. LC-MS (ESI): mass calcd. for C₁₀H₆ClN₃O, 219.02; m/z found, 220.1 [M+H]⁺. ¹H NMR (400 MHZ, DMSO-d₆) δ 10.31 (s, 1H), 7.93 (d, J=2.8 Hz, 1H), 7.86 (s, 1H), 7.11 (d, J=2.8 Hz, 1H), 5.57 (s, 2H).

### Step D: 2-(6-(2-(2,6-dioxopiperidin-3-yl)-1-oxoisoindolin-5-yl)-4-(pyrrolidin-1-ylmethyl)-1H-pyrrolo[2,3-b]pyridin-1-yl) acetonitrile

The title compound was prepared in a manner analogous to Example 1 by reductive amination between pyrrolidine and 2-(6-chloro-4-formyl-1H-pyrrolo[2,3-b]pyridin-1-yl) acetonitrile followed by Suzuki coupling with 6-chloro-1-cyclobutyl-1H-pyrrolo[2,3-b]pyridine-4-carbaldehyde (Intermediate 13) as a white solid. LC-MS (ESI): mass calcd. for C₂₇H₂₆N₆O₃, 482.21; m/z found, 483.3 [M+H]⁺. ¹H NMR (400 MHZ, DMSO-d₆) δ 11.01 (s, 1H), 8.42-8.35 (m, 2H), 8.18 (s, 1H), 7.86 (d, J=8.4 Hz, 2H), 7.66 (s, 1H), 6.78 (s, 1H), 5.58 (s, 2H), 5.17-5.13 (m, 1H), 4.58 (d, J=17.2 Hz, 1H), 4.45 (d, J=17.2 Hz, 1H), 3.97 (s, 2H), 2.97-2.87 (m, 1H), 2.67-2.55 (m, 5H), 2.45-2.33 (m, 1H), 2.10-2.01 (m, 1H), 1.74 (s, 4H).

### Example 134:3-(1-oxo-5-(4-(pyrrolidin-1-ylmethyl)-3-(trifluoromethyl)-1H-pyrazolo[3,4-b]pyridin-6-yl) isoindolin-2-yl) piperidine-2,6-dione

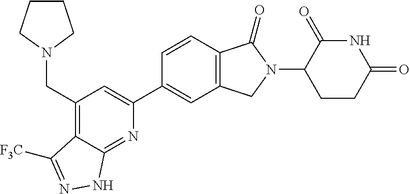 [SMILES: O=C1CCC(N2Cc3cc(-c4cc(CN5CCCC5)c5c(C(F)(F)F)n[nH]c5n4)ccc3C2=O)C(=O)N1]

### Step A: ethyl 6-chloro-3-iodo-1H-pyrazolo[3,4-b]pyridine-4-carboxylate

To a solution of ethyl 6-chloro-1H-pyrazolo[3,4-b]pyridine-4-carboxylate (5.00 g, 22.2 mmol, 1.0 eq) in DMF (100 mL) were added K₂CO₃ (3.06 g, 22.2 mmol, 1.0 eq) and I₂ (5.62 g, 22.2 mmol, 1.0 eq) and the mixture was stirring at 25° C. for 16 h. The mixture was quenched with saturated aqueous Na₂SO₃ solution (100 mL), poured into water (50 mL), and extracted with EA (50 mL×3). The organic layer was washed with brine (50 mL×4), dried over MgSO₄, filtered and concentrated under reduced pressure. The residue was purified by flash column chromatography on silica gel (PE/EA=5/1 v/v) to obtain ethyl 6-chloro-3-iodo-1H-pyrazolo[3,4-b]pyridine-4-carboxylate (5.00 g, yield 58%) as a yellow solid. LC-MS (ESI): mass calced for: C₉H₇ClIN₃O₂ 351.1; m/z found, 352.1 [M+H]⁺. ¹H NMR (400 MHZ, DMSO-d6) δ 14.61 (s, 1H), 7.55 (s, 1H), 4.48 (q, J=7.2 Hz, 2H), 1.42 (t, J=7.2 Hz, 3H).

### Step B: ethyl 6-chloro-3-iodo-1-((2-(trimethylsilyl)ethoxy)methyl)-1H-pyrazolo[3,4-b]pyridine-4-carboxylate

To a solution of ethyl 6-chloro-3-iodo-1H-pyrazolo[3,4-b]pyridine-4-carboxylate (4.20 g, 11.9 mmol, 1.0 eq) in THF (100 mL) was added Cs₂CO₃ (7.79 g, 23.9 mmol, 2.0 eq) and the mixture was stirring at 0° C. for 10 min. Then SEM-C₁ (2.99 g, 3.17 mL, 17.9 mmol, 1.5 eq) was added to above mixture and the mixture was stirred at 0° C. for 1 h. The mixture was quenched with water (20 mL) and extracted with EA (20 mL×3). The organic layer was washed with brine (20 mL×4), dried over MgSO₄, filtered and concentrated in vacuum. The residue was purified by flash column chromatography on silica gel (PE/EA=10/1 v/v) to obtain ethyl 6-chloro-3-iodo-1-((2-(trimethylsilyl)ethoxy)methyl)-1H-pyrazolo[3,4-b]pyridine-4-carboxylate (4.00 g, yield 63%) as a yellow solid. LC-MS (ESI): mass calced for: C₁₅H₂₁ClIN₃O₃Si 481.1; m/z found, 482.1 [M+H]⁺.

¹HNMR (400 MHZ, DMSO-d₆) δ 7.65 (s, 1H), 5.74 (s, 2H), 4.48 (q, J=7.2 Hz, 2H), 3.63-3.55 (m, 2H), 1.41 (t, J=7.2 Hz, 3H), 0.87-0.78 (m, 2H),-0.09 (s, 9H).

### Step C: ethyl 6-chloro-3-(trifluoromethyl)-1-((2-(trimethylsilyl)ethoxy)methyl)-1H-pyrazolo[3,4-b]pyridine-4-carboxylate

To a solution of ethyl 6-chloro-3-iodo-1-((2-(trimethylsilyl)ethoxy)methyl)-1H-pyrazolo[3,4-b]pyridine-4-carboxylate (2.00 g, 4.15 mmol, 1.0 eq) in DMF (20.0 mL) were added copper (I) iodide (2.37 g, 12.5 mmol, 3.0 eq) and methyl 2,2-difluoro-2-(fluorosulfonyl)acetate (2.39 g, 12.5 mmol, 3.0 eq). The mixture was stirred under N₂ at 80° C. for 16 h. The reaction mixture was cooled to room temperature, filtered and the filtrate was concentrated under reduced pressure. The residue was poured into water (5 mL) and extracted with EtOAc (10 mL×3). The organic layer was washed with brine (10 mL×3), dried over MgSO₄, filtered and concentrated under reduced pressure. The residue was purified by flash column chromatography on silica gel (PE/EA=10/1 v/v) to obtain ethyl 6-chloro-3-(trifluoromethyl)-1-((2-(trimethylsilyl)ethoxy)methyl)-1H-pyrazolo[3,4-b]pyridine-4-carboxylate (1.30 g, yield 66%) as a white oil. LC-MS (ESI): mass calced for: C₁₆H₂₁CIF3N₃O₃Si 423.1; m/z found, 424.1 [M+H]⁺.

### Step D: (6-chloro-3-(trifluoromethyl)-1-((2-(trimethylsilyl)ethoxy)methyl)-1H-pyrazolo[3,4-b]pyridin-4-yl) methanol

To a solution of ethyl 6-chloro-3-(trifluoromethyl)-1-((2-(trimethylsilyl)ethoxy)methyl)-1H-pyrazolo[3,4-b]pyridine-4-carboxylate (900 mg, 2.12 mmol, 1.0 eq) in THF (10.0 mL) was added dropwise diisobutylaluminum hydride (1.5 M in THF) (5.66 mL, 8.49 mmol, 4.0 eq) under N₂ at −78° C. and the mixture was stirred at this temperature for 10 min. Then the mixture was warmed to 0° C. and stirred for 1 h. The reaction mixture was quenched with H₂O (5 mL) at 0° C., diluted with water (20 mL), and extracted with EtOAc (20 mL×3). The organic layer was dried over MgSO₄, filtered and concentrated in vacuum. The residue was purified by flash column chromatography on silica gel (PE/EA=1/1 v/v) to obtain (6-chloro-3-(trifluoromethyl)-1-((2-(trimethylsilyl)ethoxy)methyl)-1H-pyrazolo[3,4-b]pyridin-4-yl) methanol (800 mg, yield 89%) as a white solid. LC-MS (ESI): mass calced for: C₁₄H₁₉CIF3N₃O₂Si 381.1; m/z found, 382.1 [M+H]⁺.

### Step E: 6-chloro-3-(trifluoromethyl)-1-((2-(trimethylsilyl)ethoxy)methyl)-1H-pyrazolo[3,4-b]pyridine-4-carbaldehyde

To a solution of (6-chloro-3-(trifluoromethyl)-1-((2-(trimethylsilyl)ethoxy)methyl)-1H-pyrazolo[3,4-b]pyridin-4-yl) methanol (700 mg, 1.83 mmol, 1.0 eq) in DMSO (15 mL) was added IBX (45 w.t. %) (2.8 g, 5.5 mmol, 3.0 eq) in portions at room temperature and the mixture was stirred at room temperature for 1 h. The reaction mixture was quenched with saturated aqueous NaHCO₃ solution (50 mL) at 0° C. and extracted with EA (35 mL×3). The organic layer was washed with H₂O (35 mL×3) and brine (35 mL), dried over anhydrous Na₂SO₄, filtered and concentrated under reduced pressure. The residue was purified by flash column chromatography on silica gel (EA/PE=1/5 v/v) to give 6-chloro-3-(trifluoromethyl)-1-((2-(trimethylsilyl)ethoxy)methyl)-1H-pyrazolo[3,4-b]pyridine-4-carbaldehyde (600 mg, yield 78%) as a yellow oil. LC-MS (ESI): mass calced for: C₁₄H₁₇ClF₃N₃O₂Si 379.1; m/z found, 380.1 [M+H]⁺. ¹H NMR (400 MHZ, CDCl₃) δ 10.56 (s, 1H), 7.83 (s, 1H), 5.96 (s, 2H), 3.76 (t, J=7.6 Hz, 2H), 0.98 (t, J=7.6 Hz, 2H), 0.00 (s, 9H).

### Step F: 6-chloro-4-(pyrrolidin-1-ylmethyl)-3-(trifluoromethyl)-1-((2-(trimethylsilyl)ethoxy)methyl)-1H-pyrazolo[3,4-b]pyridine

To a solution of 6-chloro-3-(trifluoromethyl)-1-((2-(trimethylsilyl)ethoxy)methyl)-1H-pyrazolo[3,4-b]pyridine-4-carbaldehyde (400 mg, 1.05 mmol, 1.0 eq) and pyrrolidine (150 mg, 173 μL, 2.11 mmol, 2.0 eq) in DCM (10 mL) was added AcOH (0.10 mL, 1.7 mmol, 1.7 Eq) and the mixture was stirred at room temperature for 1 h. Then sodium triacetoxyborohydride (670 mg, 3.16 mmol, 3.0 eq) was added to above mixture and the mixture was stirred at room temperature overnight. The mixture was diluted with DCM (30 mL), washed with brine (30 mL×2), dried over anhydrous Na₂SO₄, filtered and concentrated under reduced pressure. The residue was purified by flash column chromatography on silica gel (EA/PE=1/5 v/v) to give 6-chloro-4-(pyrrolidin-1-ylmethyl)-3-(trifluoromethyl)-1-((2-(trimethylsilyl)ethoxy)methyl)-1H-pyrazolo[3,4-b]pyridine (400 mg, yield 79%) as a yellow oil. LC-MS (ESI): mass calced for: C₁₈H₂₆ClF₃N₄OSi 434.1; m/z found, 435.1 [M+H]⁺. ¹H NMR (400 MHZ, CDCl₃) δ 7.60 (s, 1H), 5.89 (s, 2H), 4.03 (s, 2H), 3.74 (t, J=8.0 Hz, 2H), 2.68 (s, 4H), 1.89 (s, 4H), 0.98 (t, J=8.0 Hz, 2H), 0.00 (s, 9H).

### Step G: 3-(1-oxo-5-(4-(pyrrolidin-1-ylmethyl)-3-(trifluoromethyl)-1-((2-(trimethylsilyl) ethoxy)methyl)-1H-pyrazolo[3,4-b]pyridin-6-yl) isoindolin-2-yl) piperidine-2,6-dione

To a solution of 6-chloro-4-(pyrrolidin-1-ylmethyl)-3-(trifluoromethyl)-1-((2-(trimethylsilyl)ethoxy)methyl)-1H-pyrazolo[3,4-b]pyridine (100 mg, 230 μmol, 1.0 eq) in dioxane (5 mL) and water (0.5 mL) were added 3-(1-oxo-5-(4,4,5,5-tetramethyl-1,3,2-dioxaborolan-2-yl) isoindolin-2-yl) piperidine-2,6-dione (Intermediate 13, 102 mg, 276 μmol, 1.2 eq), Pd (dtbpf) Cl₂ (15.0 mg, 23.0 μmol, 0.1 eq), and potassium phosphate (146 mg, 690 μmol, 3.0 eq). The mixture was stirred under N₂ at 95° C. for 1 h. After cooled to room temperature, the mixture was filtered and the cake was washed with EA (20 mL). The filtrate was concentrated under reduced pressure and the residue was purified by Prep-TLC(100% EA, v/v) to give 3-(1-oxo-5-(4-(pyrrolidin-1-ylmethyl)-3-(trifluoromethyl)-1-((2-(trimethylsilyl)ethoxy)methyl)-1H-pyrazolo[3,4-b]pyridin-6-yl) isoindolin-2-yl) piperidine-2,6-dione (140 mg, yield 85%) as a yellow oil. LC-MS (ESI): mass calced for: C₃₁H₃₇F₃N₆₀₄Si 642.2; m/z found, 643.1 [M+H]⁺.

### Step H: 3-(1-oxo-5-(4-(pyrrolidin-1-ylmethyl)-3-(trifluoromethyl)-1H-pyrazolo[3,4-b]pyridin-6-yl) isoindolin-2-yl) piperidine-2,6-dione

To a solution of 3-(1-oxo-5-(4-(pyrrolidin-1-ylmethyl)-3-(trifluoromethyl)-1-((2-(trimethylsilyl)ethoxy)methyl)-1H-pyrazolo[3,4-b]pyridin-6-yl) isoindolin-2-yl) piperidine-2,6-dione (130 mg, 202 μmol, 1.0 eq) in DCM (5.0 mL) was added dropwise TFA (1.48 g, 1.00 mL, 13.0 mmol, 65 eq) at 0° C. and the mixture was stirred at room temperature for 16 hours. The mixture was poured into water (6 mL) and extracted with DCM (10 mL×3). The organic layer was dried over MgSO₄, filtered and concentrated under reduced pressure. The residue was purified by Prep-TLC(DCM/MeOH=10/1 v/v) to give 3-(1-oxo-5-(4-(pyrrolidin-1-ylmethyl)-3-(trifluoromethyl)-1H-pyrazolo[3,4-b]pyridin-6-yl) isoindolin-2-yl) piperidine-2,6-dione (30.3 mg, yield 28%) as a white solid. LC-MS (ESI): mass calced for: C₂₅H₂₃F₃N_{603 512.1}; m/z found, 513.1 [M+H]⁺. ¹H NMR (400 MHZ, DMSO-d₆) δ 14.76 (s, 1H), 11.00 (s, 1H), 8.38 (s, 1H), 8.31 (d, J=8.0 Hz, 1H), 8.12 (d, J=11.6 Hz, 1H), 7.91 (d, J=8.0 Hz, 1H), 5.17-5.12 (m, 1H), 4.58 (d, J=17.6 Hz, 1H), 4.46 (d, J=17.6 Hz, 1H), 4.08 (s, 2H), 2.94 (dd, J=13.8, 5.4 Hz, 1H), 2.74-2.58 (m, 5H), 2.45-2.37 (m, 1H), 2.07-2.00 (m, 1H), 1.83 (s, 4H). ¹⁹F NMR (400 MHZ, DMSO-d₆) δ-73.47 (ppm).

### Example 135:3-(5-(1-cyclopropyl-4-((3-hydroxyazetidin-1-yl)methyl)-1H-pyrrolo[2,3-b]pyridin-6-yl)-1-oxoisoindolin-2-yl) piperidine-2,6-dione

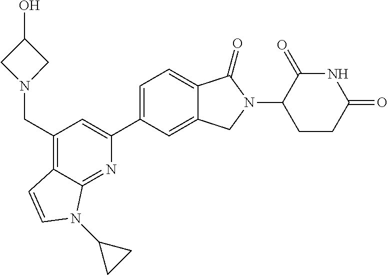 [SMILES: O=C1CCC(N2Cc3cc(-c4cc(CN5CC(O)C5)c5ccn(C6CC6)c5n4)ccc3C2=O)C(=O)N1]

The title compound was prepared in a manner analogous to Example 1 by reductive amination between azetidin-3-ol and 6-chloro-1-cyclopropyl-1H-pyrrolo[2,3-b]pyridine-4-carbaldehyde (Intermediate 30) followed by Suzuki coupling with 6-chloro-1-cyclobutyl-1H-pyrrolo[2,3-b]pyridine-4-carbaldehyde (Intermediate 13) as a yellow solid. LC-MS (ESI): mass calced for: C₂₇H₂₇N₅O₄ 485.2; m/z found, 486.1 [M+H]⁺. ¹H NMR (400 MHZ, DMSO-d6) § 11.01 (s, 1H), 8.36 (s, 1H), 8.34 (d, J=8.0 Hz, 1H), 8.14 (s, 1H), 7.86 (d, J=8.0 Hz, 1H), 7.77 (s, 1H), 7.54 (d, J=3.6 Hz, 1H), 6.62 (d, J=3.6 Hz, 1H), 5.56 (s, 1H), 5.18-5.13 (m, 1H), 4.58 (d, J=17.2 Hz, 1H), 4.45 (d, J=17.2 Hz, 1H), 4.38-4.25 (m, 1H), 4.13 (s, 2H), 3.78-3.72 (m, 4H), 3.17-3.13 (m, 1H), 2.98-2.86 (m, 1H), 2.63 (d, J=16.8 Hz, 1H), 2.47-2.35 (m, 1H), 2.09-2.03 (m, 1H), 1.12-1.08 (m, 4H).

### Example 136:3-(5-(1-(2-(methylsulfonyl)ethyl)-4-(pyrrolidin-1-ylmethyl)-1H-pyrrolo[2,3-b]pyridin-6-yl)-1-oxoisoindolin-2-yl) piperidine-2,6-dione

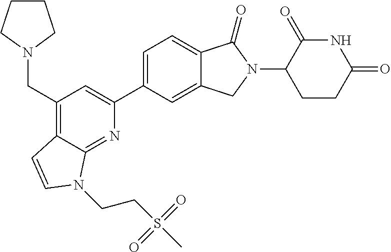 [SMILES: CS(=O)(=O)CCn1ccc2c(CN3CCCC3)cc(-c3ccc4c(c3)CN(C3CCC(=O)NC3=O)C4=O)nc21]

### Step A: methyl 6-chloro-1-(2-(methylsulfonyl)ethyl)-1H-pyrrolo[2,3-b]pyridine-4-carboxylate

To a solution of 1-bromo-2-(methylsulfonyl) ethane (666 mg, 3.6 mmol, 1.5 eq) and methyl 6-chloro-1H-pyrrolo[2,3-b]pyridine-4-carboxylate (500 mg, 2.4 mmol, 1.0 eq) in DMF (10.0 mL) was added Cs₂CO₃ (1.6 g, 4.8 mmol, 2.0 eq). The mixture was stirred under N₂ at 70° C. for 3 h. The resulting mixture was cooled to room temperature and diluted with EA (100 mL). The mixture was washed with brine (100 mL×4), dried over anhydrous Na₂SO₄, filtered and concentrated in vacuum. The residue was purified by flash column chromatography on silica gel (PE/EA=3/1 v/v) to give methyl 6-chloro-1-(2-(methylsulfonyl)ethyl)-1H-pyrrolo[2,3-b]pyridine-4-carboxylate (430 mg, yield 57%) as an off-white solid. LC-MS (ESI): mass calcd. for C₁₂H₁₃ClN₂O₄S, 316.03; m/z found, 317.0 [M+H]⁺.

### Step B: (6-chloro-1-(2-(methylsulfonyl)ethyl)-1H-pyrrolo[2,3-b]pyridin-4-yl) methanol

To a solution of methyl 6-chloro-1-(2-(methylsulfonyl)ethyl)-1H-pyrrolo[2,3-b]pyridine-4-carboxylate (220 mg, 695 μmol, 1.0 eq) in THF (3.0 mL) was added DIBAL-H (2 M in THF) (0.7 mL, 1.4 mmol, 2.0 eq) under N₂ at 0° C. The reaction mixture was stirred at 0° C. for 2 h. The reaction mixture was quenched with water (20 mL) and extracted with DCM (30 mL×3). The organic layer was washed with brine (30 mL), dried over Na₂SO₄, filtered and concentrated under reduced pressure. The residue was purified by flash column chromatography on silica gel (PE/EA=20/1 v/v) to give (6-chloro-1-(2-(methylsulfonyl)ethyl)-1H-pyrrolo[2,3-b]pyridin-4-yl) methanol (180 mg, yield 90%) as a yellow solid. LC-MS (ESI): mass calcd. for C₁₁H₁₃ClN₂O₃S, 288.0; m/z found, 289.0 [M+H]⁺.

### Step C: 6-chloro-1-(2-(methylsulfonyl)ethyl)-1H-pyrrolo[2,3-b]pyridine-4-carbaldehyde

To a solution of (6-chloro-1-(2-(methylsulfonyl)ethyl)-1H-pyrrolo[2,3-b]pyridin-4-yl) methanol (200 mg, 693 μmol, 1.0 eq) in DMSO (5.0 mL) was added IBX (Purity 45% W.t) (388 mg, 1.39 mmol, 2.0 eq) at room temperature and the reaction mixture was stirred at room temperature for 20 min. The reaction mixture was quenched with water (20 mL) and extracted with EA (30 mL×3). The organic layer was washed with brine (30 mL×3), dried over Na₂SO₄, filtered and concentrated under reduced pressure. The residue was purified by flash column chromatography on silica gel (PE/EA=10/1 v/v) to give 6-chloro-1-(2-(methylsulfonyl)ethyl)-1H-pyrrolo[2,3-b]pyridine-4-carbaldehyde (120 mg, yield 60%) as a yellow oil. LC-MS (ESI): mass calcd. for C₁H₁ClN₂O₃S, 286.0; m/z found, 287.0 [M+H]⁺.

### Step D: 3-(5-(1-(2-(methylsulfonyl)ethyl)-4-(pyrrolidin-1-ylmethyl)-1H-pyrrolo[2,3-b]pyridin-6-yl)-1-oxoisoindolin-2-yl) piperidine-2,6-dione

The title compound was prepared in a manner analogous to Example 1 by reductive amination between pyrrolidine and 6-chloro-1-(2-(methylsulfonyl)ethyl)-1H-pyrrolo[2,3-b]pyridine-4-carbaldehyde followed by Suzuki coupling with 6-chloro-1-cyclobutyl-1H-pyrrolo[2,3-b]pyridine-4-carbaldehyde (Intermediate 13) as a brown solid. LC-MS (ESI): mass calcd. For C₂₈H₃₁N₅O₅S, 549.2; m/z found, 550.2 [M+H]⁺. ¹H NMR (400 MHZ, DMSO-d₆) δ 1.07 (s, 1H), 8.45 (s, 1H), 8.40 (d, J=8.0 Hz, 1H), 8.23 (s, 1H), 7.98 (s, 1H), 7.91 (d, J=8.0 Hz, 1H), 7.74 (d, J=3.6 Hz, 1H), 6.79 (d, J=3.6 Hz, 1H), 5.22 (dd, J=13.2, 5.2 Hz, 1H), 4.85 (t, J=6.8 Hz, 2H), 4.63 (d, J=17.2 Hz, 1H), 4.50 (d, J=17.2 Hz, 1H), 4.25 (s, 2H), 3.89 (d, J=6.8 Hz, 2H), 3.05 (s, 3H), 2.99 (dd, J=10.8, 6.8 Hz, 1H), 2.83 (s, 4H), 2.68 (d, J=16.4 Hz, 1H), 2.53-2.44 (m, 1H), 2.16-2.05 (m, 1H), 1.87 (s, 4H).

### Example 137:3-(5-(4-((3-hydroxy-4-isopropylpyrrolidin-1-yl)methyl)-1H-pyrrolo[2,3-b]pyridin-6-yl)-1-oxoisoindolin-2-yl) piperidine-2,6-dione

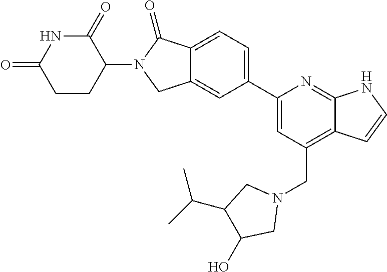 [SMILES: CC(C)C1CN(Cc2cc(-c3ccc4c(c3)CN(C3CCC(=O)NC3=O)C4=O)nc3[nH]ccc23)CC1O]

The title compound was prepared in a manner analogous to Example 1 by reductive amination between of 4-isopropylpyrrolidin-3-ol and 6-chloro-1H-pyrrolo[2,3-b]pyridine-4-carbaldehyde (Intermediate 9) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₈H₃₁N₅O₄, 501.2; m/z found, 502.2 [M+H]⁺.

¹H NMR (400 MHZ, DMSO-d₆) δ 11.82 (s, 1H), 11.07 (s, 1H), 8.36 (s, 1H), 8.30 (d, J=8.0 Hz, 1H), 8.22 (s, 1H), 7.89 (d, J=8.0 Hz, 1H), 7.81 (s, 1H), 7.60-7.55 (m, 1H), 6.70 (dd, J=3.2, 1.6 Hz, 1H), 5.23-5.18 (m, 1H), 4.62 (d, J=17.2 Hz, 1H), 4.49 (d, J=17.2 Hz, 1H), 4.06-3.93 (m, 3H), 3.02-2.94 (m, 2H), 2.73-2.64 (m, 3H), 2.48 (tt, J=13.2, 6.8 Hz, 1H), 2.27 (t, J=8.8 Hz, 1H), 2.17-2.05 (m, 1H), 1.78-1.70 (m, 1H), 1.62 (dd, J=13.2, 6.8 Hz, 1H), 0.99 (d, J=6.8 Hz, 3H), 0.88 (t, J=5.2 Hz, 3H).

### Example 138:3-(5-(4-((3-hydroxy-4-isobutylpyrrolidin-1-yl)methyl)-1H-pyrrolo[2,3-b]pyridin-6-yl)-1-oxoisoindolin-2-yl) piperidine-2,6-dione

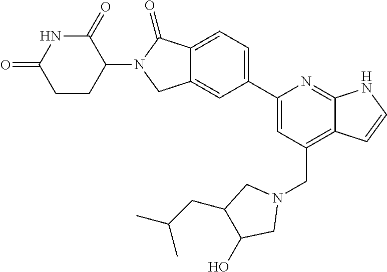 [SMILES: CC(C)CC1CN(Cc2cc(-c3ccc4c(c3)CN(C3CCC(=O)NC3=O)C4=O)nc3[nH]ccc23)CC1O]

The title compound was prepared in a manner analogous to Example 1 by reductive amination between of 4-isobutylpyrrolidin-3-ol and 6-chloro-1H-pyrrolo[2,3-b]pyridine-4-carbaldehyde (Intermediate 9) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₉H₃₃N₅O₄, 515.3; m/z found, 516.3 [M+H]⁺. ¹H NMR (400 MHZ, DMSO-d₆) δ 11.76 (s, 1H), 11.01 (s, 1H), 8.30 (s, 1H), 8.25 (d, J=8.0 Hz, 1H), 8.17 (s, 1H), 7.83 (d, J=8.0 Hz, 1H), 7.74 (s, 1H), 7.54-7.47 (m, 1H), 6.64 (dd, J=3.2, 2.0 Hz, 1H), 5.18-5.13 (m, 1H), 4.56 (d, J=17.2 Hz, 1H), 4.43 (d, J=17.2 Hz, 1H), 4.02-3.89 (m, 2H), 3.79-3.75 (m, 1H), 2.99-2.89 (m, 2H), 2.77-2.70 (m, 1H), 2.66-2.57 (m, 2H), 2.46-2.36 (m, 1H), 2.21-2.15 (m, 1H), 2.08-1.94 (m, 2H), 1.54 (td, J=13.2, 6.8 Hz, 1H), 1.35 (dt, J=13.8, 7.2 Hz, 1H), 1.18 (dd, J=7.6, 5.6 Hz, 1H), 0.90-0.79 (m, 6H).

### Example 139:3-(1-oxo-5-(1-phenyl-4-(pyrrolidin-1-ylmethyl)-1H-pyrrolo[2,3-b]pyridin-6-yl) isoindolin-2-yl) piperidine-2,6-dione

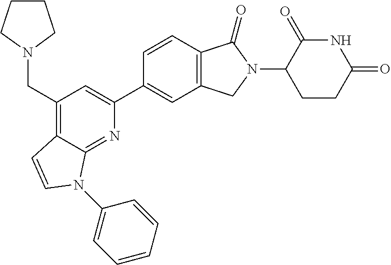 [SMILES: O=C1CCC(N2Cc3cc(-c4cc(CN5CCCC5)c5ccn(-c6ccccc6)c5n4)ccc3C2=O)C(=O)N1]

The title compound was prepared in a manner analogous to Example 1 by reductive amination between of pyrrolidine and 6-chloro-1-phenyl-1H-pyrrolo[2,3-b]pyridine-4-carbaldehyde (Intermediate 43) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₃₁H₂₉N₅O₃ 519.23; m/z found, 520.2 [M+H]⁺. ¹H NMR (400 MHz, DMSO-d₆) δ 11.01 (s, 1H), 8.34 (s, 1H), 8.29 (d, J=8.0 Hz, 1H), 8.15 (s, 1H), 8.04-8.00 (m, 2H), 7.99 (s, 2H), 7.84 (d, J=8.0 Hz, 1H), 7.61 (t, J=8.0 Hz, 2H), 7.40 (t, J=7.4 Hz, 1H), 6.96 (d, J=3.6 Hz, 1H), 5.18-5.13 (m, 1H), 4.57 (d, J=17.4 Hz, 1H), 4.43 (d, J=17.4 Hz, 1H), 4.20 (s, 2H), 2.97-2.90 (m, 1H), 2.77 (s, 4H), 2.62 (d, J=16.8 Hz, 1H), 2.45-2.40 (m, 1H), 2.08-2.01 (m, 1H), 1.82 (s, 4H).

### Example 140:3-(5-(1-isobutyl-4-(pyrrolidin-1-ylmethyl)-1H-pyrrolo[2,3-b]pyridin-6-yl)-1-oxoisoindolin-2-yl) piperidine-2,6-dione

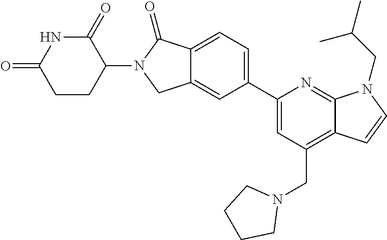 [SMILES: CC(C)Cn1ccc2c(CN3CCCC3)cc(-c3ccc4c(c3)CN(C3CCC(=O)NC3=O)C4=O)nc21]

The title compound was prepared in a manner analogous to Example 1 by reductive amination between of pyrrolidine and -chloro-1-isobutyl-1H-pyrrolo[2,3-b]pyridine-4-carbaldehyde (Intermediate 44) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. For C₂₉H₃₃N₅O₃, 499.26; m/z found, 500.2 [M+H]⁺. ¹H NMR (400 MHZ, DMSO-d₆) δ 11.02 (s, 1H), 8.34 (s, 1H), 8.30 (d, J=8.0 Hz, 1H), 8.21 (s, 1H), 7.84 (d, J=8.0 Hz, 1H), 7.80 (s, 1H), 7.59 (d, J=3.2 Hz, 1H), 6.66 (d, J=3.2 Hz, 1H), 5.18-5.13 (m, 1H), 4.58 (d, J=17.4 Hz, 1H), 4.44 (d, J=17.4 Hz, 1H), 4.17 (d, J=7.2 Hz, 2H), 4.05 (s, 2H), 3.02-2.87 (m, 1H), 2.68-2.56 (m, 5H), 2.48-2.36 (m, 1H), 2.32-2.23 (m, 1H), 2.11-1.99 (m, 1H), 1.83-1.71 (m, 4H), 0.90 (d, J=6.6 Hz, 6H).

### Example 141:3-(5-(1-cyclopropyl-4-((3-fluoropyrrolidin-1-yl)methyl)-1H-pyrrolo[2,3-b]pyridin-6-yl)-1-oxoisoindolin-2-yl) piperidine-2,6-dione

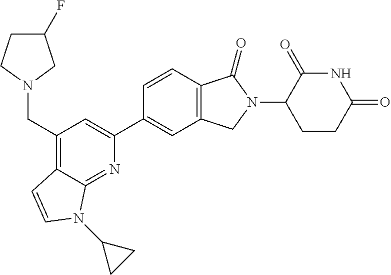 [SMILES: O=C1CCC(N2Cc3cc(-c4cc(CN5CCC(F)C5)c5ccn(C6CC6)c5n4)ccc3C2=O)C(=O)N1]

The title compound was prepared in a manner analogous to Example 1 by reductive amination between of 3-fluoropyrrolidine and 6-chloro-1-cyclopropyl-1H-pyrrolo[2,3-b]pyridine-4-carbaldehyde (Intermediate 30) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₈H₂₈FN₅O₃, 501.56; m/z found, 502.2 [M+H]⁺. ¹H NMR (400 MHZ, DMSO-d₆) δ 11.02 (s, 1H), 10.75 (d, J=113.0 Hz, 1H), 8.38 (s, 1H), 8.35 (d, J=8.0 Hz, 1H), 8.06 (s, 1H), 7.91 (d, J=8.0 Hz, 1H), 7.70 (d, J=3.6 Hz, 1H), 6.80 (d, J=3.6 Hz, 1H), 5.49 (d, J=52.6 Hz, 1H), 5.19-5.14 (m, 1H), 4.77 (s, 2H), 4.60 (d, J=17.4 Hz, 1H), 4.47 (d, J=17.4 Hz, 1H), 3.81-3.76 (m, 3H), 3.52 (s, 1H), 3.37 (s, 1H), 3.01-2.86 (m, 1H), 2.64 (d, J=17.8 Hz, 1H), 2.51 (s, 1H), 2.49-2.39 (m, 1H), 2.23 (s, 1H), 2.10-2.01 (m, 1H), 1.19-1.06 (m, 4H). 19¹H NMR (400 MHz, DMSO-d₆) δ-73.75,-170.22 (ppm).

### Example 142:3-(5-(1-cyclopropyl-4-((3-hydroxypyrrolidin-1-yl)methyl)-1H-pyrrolo[2,3-b]pyridin-6-yl)-1-oxoisoindolin-2-yl) piperidine-2,6-dione

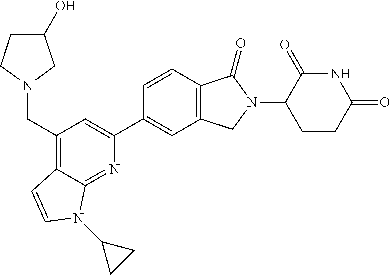 [SMILES: O=C1CCC(N2Cc3cc(-c4cc(CN5CCC(O)C5)c5ccn(C6CC6)c5n4)ccc3C2=O)C(=O)N1]

The title compound was prepared in a manner analogous to Example 1 by reductive amination between of pyrrolidin-3-ol and 6-chloro-1-cyclopropyl-1H-pyrrolo[2,3-b]pyridine-4-carbaldehyde (Intermediate 30) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₈H₂₉N₅O₄ 499.57; m/z found, 500.2 [M+H]⁺. ¹H NMR (400 MHZ, DMSO-d₆) δ 11.02 (s, 1H), 10.45 (d, J=86.2 Hz, 1H), 8.38 (s, 1H), 8.35 (d, J=8.2 Hz, 1H), 8.07 (d, J=12.2 Hz, 1H), 7.91 (d, J=8.0 Hz, 1H), 7.69 (d, J=3.2 Hz, 1H), 6.79 (s, 1H), 5.52 (d, J=44.4 Hz, 1H), 5.19-5.14 (m, 1H), 4.75 (d, J=27.6 Hz, 2H), 4.60 (d, J=17.4 Hz, 1H), 4.47 (d, J=17.4 Hz, 2H), 3.80-3.76 (m, 1H), 3.59 (s, 1H), 3.44 (s, 1H), 3.13 (s, 2H), 3.01-2.89 (m, 1H), 2.64 (d, J=16.8 Hz, 1H), 2.49-2.27 (m, 2H), 2.08-2.02 (m, 1H), 1.93 (d, J=49.2 Hz, 1H), 1.17-1.08 (m, 4H). ¹⁹F NMR (400 MHZ, DMSO-d₆) δ -73.12 (ppm).

### Example 143:3-(5-(1-cyclopropyl-4-((3-fluoroazetidin-1-yl)methyl)-1H-pyrrolo[2,3-b]pyridin-6-yl)-1-oxoisoindolin-2-yl) piperidine-2,6-dione

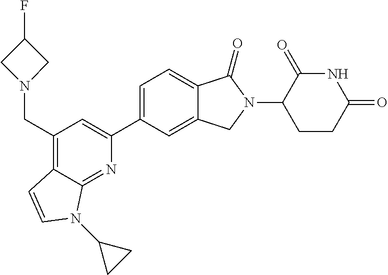 [SMILES: O=C1CCC(N2Cc3cc(-c4cc(CN5CC(F)C5)c5ccn(C6CC6)c5n4)ccc3C2=O)C(=O)N1]

The title compound was prepared in a manner analogous to Example 1 by reductive amination between of 3-fluoroazetidine hydrochloride and 6-chloro-1-cyclopropyl-1H-pyrrolo[2,3-b]pyridine-4-carbaldehyde (Intermediate 30) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₇H₂₆FN₅O₃, 487.2; m/z found, 488.1 [M+H]⁺. ¹H NMR (400 MHZ, DMSO-d₆) δ 11.01 (s, 1H), 8.36 (s, 1H), 8.34 (d, J=8.0 Hz, 1H), 7.85 (d, J=8.0 Hz, 1H), 7.72 (s, 1H), 7.51 (d, J=3.6 Hz, 1H), 6.58 (d, J=3.6 Hz, 1H), 5.35-5.09 (m, 2H), 4.58 (d, J=17.2 Hz, 1H), 4.44 (d, J=17.2 Hz, 1H), 4.00 (s, 2H), 3.77-3.59 (m, 3H), 3.28-3.22 (m, 2H), 2.98-2.86 (m, 1H), 2.63 (d, J=16.2 Hz, 1H), 2.44 (dd, J=13.2, 4.4 Hz, 1H), 2.11-1.99 (m, 1H), 1.12-1.04 (m, 4H).

### Example 144:3-(5-(1-cyclopropyl-4-((4-(hydroxymethyl) piperidin-1-yl)methyl)-1H-pyrrolo[2,3-b]pyridin-6-yl)-1-oxoisoindolin-2-yl) piperidine-2,6-dione

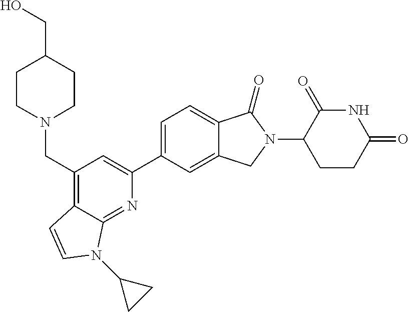 [SMILES: O=C1CCC(N2Cc3cc(-c4cc(CN5CCC(CO)CC5)c5ccn(C6CC6)c5n4)ccc3C2=O)C(=O)N1]

The title compound was prepared in a manner analogous to Example 1 by reductive amination between of piperidin-4-ylmethanol and 6-chloro-1-cyclopropyl-1H-pyrrolo[2,3-b]pyridine-4-carbaldehyde (Intermediate 30) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calced for: C₃₀H₃₃N₅O₄, 527.2; m/z found, 528.1 [M+H]⁺. ¹H NMR (400 MHZ, DMSO-d₆) δ 11.01 (s, 1H), 8.36 (s, 1H), 8.33 (d, J=8.2 Hz, 1H), 7.85 (d, J=8.0 Hz, 1H), 7.74 (s, 1H), 7.49 (s, 1H), 6.63 (s, 1H), 5.18-5.13 (m, 1H), 4.58 (d, J=17.4 Hz, 1H), 4.44 (d, J=17.4 Hz, 2H), 3.85-3.68 (m, 3H), 3.25 (t, J=5.4 Hz, 2H), 3.01-2.81 (m, 3H), 2.63 (d, J=17.2 Hz, 1H), 2.44 (dd, J=13.2, 4.4 Hz, 1H), 2.04 (dd, J=8.8, 3.6 Hz, 3H), 1.63 (s, 2H), 1.37 (d, J=14.4 Hz, 2H), 1.19 (s, 1H), 1.10 (d, J=7.8 Hz, 4H).

### Example 145:3-(1-oxo-5-(7-(pyrrolidin-1-ylmethyl)-3H-imidazo[4,5-b]pyridin-5-yl) isoindolin-2-yl) piperidine-2,6-dione

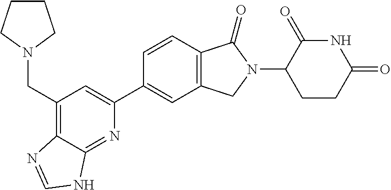 [SMILES: O=C1CCC(N2Cc3cc(-c4cc(CN5CCCC5)c5nc[nH]c5n4)ccc3C2=O)C(=O)N1]

### Step A: 5-chloro-7-(pyrrolidin-1-ylmethyl)-3-((2-(trimethylsilyl)ethoxy)methyl)-3H-imidazo[4,5-b]pyridine

To a solution of 5-chloro-3-((2-(trimethylsilyl)ethoxy)methyl)-3H-imidazo[4,5-b]pyridine-7-carbaldehyde (Intermediate 45, 70.0 mg, 224 μmol, 1.0 eq) in DCM (1.50 mL) were added pyrrolidine (19.2 mg, 269 μmol, 1.2 eq) and AcOH (0.10 mL). The mixture was stirred at 30° C. for 16 h. Sodium triacetoxyborohydride (71.4 mg, 337 μmol, 1.5 eq) was added to above mixture and the resulting reaction mixture was stirred at 30° C. for 2 h. The reaction mixture was quenched with saturated aqueous NaHCO₃ solution (5 mL) and extracted with DCM (5 mL×3). The combined organic layers were washed with brine (5 mL), dried over anhydrous Na₂SO₄, filtered and concentrated under reduced pressure. The crude product was purified by Prep-TLC(DCM/MeOH=10/1 v/v) to afford 5-chloro-7-(pyrrolidin-1-ylmethyl)-3-((2-(trimethylsilyl)ethoxy)methyl)-3H-imidazo[4,5-b]pyridine (60.0 mg, yield 72%) as a yellow oil. LC-MS (ESI): mass calced for: C₁₇H₂₇ClN₄OSi 366.16; m/z found, 367.1 [M+H]⁺.

### Step B: 3-(1-oxo-5-(7-(pyrrolidin-1-ylmethyl)-3-((2-(trimethylsilyl)ethoxy)methyl)-3H-imidazo[4,5-b]pyridin-5-yl) isoindolin-2-yl) piperidine-2,6-dione

To a solution of 5-chloro-7-(pyrrolidin-1-ylmethyl)-3-((2-(trimethylsilyl)ethoxy)methyl)-3H-imidazo[4,5-b]pyridine (60.0 mg, 164 μmol, 1.0 eq) in 1,4-Dioxane (2.00 mL) and water (0.20 mL) were added 3-(1-oxo-5-(4,4,5,5-tetramethyl-1,3,2-dioxaborolan-2-yl) isoindolin-2-yl) piperidine-2,6-dione (Intermediate 13, 72.6 mg, 196 μmol, 1.2 eq), Potassium phosphate tribasic (104 mg, 491 μmol, 3 eq), and 1,1′-Bis(di-t-butylphosphino) ferrocene palladium dichloride (10.7 mg, 16.4 μmol, 0.1 eq). The mixture was stirred under N₂ at 95° C. for 3 h. After cooled to room temperature, the reaction mixture was filtered and the filtrate was concentrated in vacuum. The crude product was purified by Prep-TLC(DCM/MeOH=7/1 v/v) to afford 3-(1-oxo-5-(7-(pyrrolidin-1-ylmethyl)-3-((2-(trimethylsilyl)ethoxy)methyl)-3H-imidazo[4,5-b]pyridin-5-yl) isoindolin-2-yl) piperidine-2,6-dione (70.0 mg, yield 74%) as a yellow solid. LC-MS (ESI): mass calced for: C₃₀H₃₈N₆₀₄Si 574.27; m/z found, 575.1 [M+H]⁺.

### Step C: 3-(1-oxo-5-(7-(pyrrolidin-1-ylmethyl)-3H-imidazo[4,5-b]pyridin-5-yl) isoindolin-2-yl) piperidine-2,6-dione

To a solution of 3-(1-oxo-5-(7-(pyrrolidin-1-ylmethyl)-3-((2-(trimethylsilyl)ethoxy)methyl)-3H-imidazo[4,5-b]pyridin-5-yl) isoindolin-2-yl) piperidine-2,6-dione (70.0 mg, 122 μmol, 1.0 eq) in DCM (1.50 mL) was added TFA (3.00 mL). The mixture was stirred at 25° C. for 16 h. After evaporation, the crude product was purified by Prep-HPLC with YMC-Actus Triart 18C(5 μm, 20×250 mm), and mobile phase of 5-35% ACN in water (0.05% TFA) over 10 min and 35-50% ACN in water over 6 min and 50-95% ACN in water over 1 min then hold at 95% ACN for 2 min, at a flow rate of 25 mL/min to afford 3-(1-oxo-5-(7-(pyrrolidin-1-ylmethyl)-3H-imidazo[4,5-b]pyridin-5-yl) isoindolin-2-yl) piperidine-2,6-dione trifluoroacetate (23.8 mg, yield 44%) as a white solid. LC-MS (ESI): mass calced for: C₂₄H₂₄N₆O₃, 444.5; m/z found, 445.1 [M+H]⁺. ¹H NMR (400 MHZ, DMSO-d₆) δ 11.03 (s, 1H), 10.35 (s, 1H), 8.67 (s, 1H), 8.44 (s, 1H), 8.32 (d, J=7.8 Hz, 1H), 7.96 (d, J=8.0 Hz, 1H), 7.82 (s, 1H), 5.20-5.16 (m, 1H), 4.67 (d, J=5.2 Hz, 2H), 4.60 (d, J=17.6 Hz, 1H), 4.48 (d, J=17.6 Hz, 1H), 3.54 (s, 2H), 3.23 (s, 2H), 3.02-2.89 (m, 1H), 2.64 (d, J=16.4 Hz, 1H), 2.49-2.40 (m, 1H), 2.14-2.00 (m, 3H), 1.93 (s, 2H). ¹⁹F NMR (400 MHZ, DMSO-d₆) δ-74.26 (ppm).

### Example 146:3-(5-(4-((3-isopropylpyrrolidin-1-yl)methyl)-1H-pyrrolo|2,3-b]pyridin-6-yl)-1-oxoisoindolin-2-yl) piperidine-2,6-dione

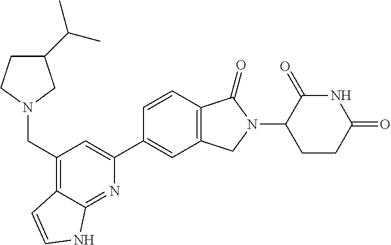 [SMILES: CC(C)C1CCN(Cc2cc(-c3ccc4c(c3)CN(C3CCC(=O)NC3=O)C4=O)nc3[nH]ccc23)C1]

The title compound was prepared in a manner analogous to Example 1 by reductive amination between of isopropylpyrrolidine hydrochloride (Intermediate 46) and 6-chloro-1H-pyrrolo[2,3-b]pyridine-4-carbaldehyde (Intermediate 9) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₈H₃₁N₅O₃, 485.2; m/z found, 486.3 [M+H]⁺. ¹H NMR (400 MHZ, DMSO-d₆) δ 11.73 (s, 1H), 11.01 (s, 1H), 8.42 (s, 1H), 8.29 (s, 1H), 8.24 (d, J=8.0 Hz, 1H), 7.82 (d, J=8.0 Hz, 1H), 7.71 (s, 1H), 7.56-7.44 (m, 1H), 6.62 (dd, J=3.4, 1.8 Hz, 1H), 5.16-5.13 (m, 1H), 4.54 (d, J=17.4 Hz, 1H), 4.44 (d, J=17.4 Hz, 1H), 3.92 (q, J=13.8 Hz, 2H), 2.92 (dd, J=21.6, 9.4 Hz, 1H), 2.80-2.61 (m, 3H), 2.45-2.37 (m, 1H), 2.22-2.16 (m, 1H), 2.11-1.75 (m, 4H), 1.44 (dd, J=13.6, 6.0 Hz, 2H), 0.84 (dd, J=18.4, 6.6 Hz, 6H).

### Example 147:3-(5-(1-benzyl-4-(pyrrolidin-1-ylmethyl)-1H-pyrrolo[2,3-b]pyridin-6-yl)-1-oxoisoindolin-2-yl) piperidine-2,6-dione

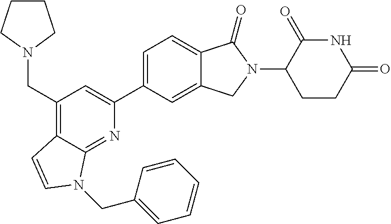 [SMILES: O=C1CCC(N2Cc3cc(-c4cc(CN5CCCC5)c5ccn(Cc6ccccc6)c5n4)ccc3C2=O)C(=O)N1]

The title compound was prepared in a manner analogous to Example 1 by reductive amination between of pyrrolidine and 1-benzyl-6-chloro-1H-pyrrolo[2,3-b]pyridine-4-carbaldehyde (Intermediate 47) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₃₂H₃₁N₅O₃, 533.24; m/z found, 534.3 [M+H]⁺. ¹H NMR (400 MHZ, DMSO-d₆) δ 11.01 (s, 1H), 8.36 (s, 1H), 8.32 (d, J=8.0 Hz, 1H), 8.15 (s, 1H), 7.83 (d, J=8.0 Hz, 1H), 7.77 (s, 1H), 7.66 (d, J=3.6 Hz, 1H), 7.37-7.30 (m, 4H), 7.25 (t, J=6.8 Hz, 1H), 6.67 (d, J=3.6 Hz, 1H), 5.57 (s, 2H), 5.18-5.13 (m, 1H), 4.57 (d, J=17.2 Hz, 1H), 4.44 (d, J=17.2 Hz, 1H), 3.96 (s, 2H), 2.98-2.89 (m, 1H), 2.62 (d, J=17.4 Hz, 1H), 2.55 (s, 4H), 2.48-2.40 (m, 1H), 2.08-2.01 (m, 1H), 1.73 (s, 4H).

### Example 148:3-(5-(1-(but-2-yn-1-yl)-4-(pyrrolidin-1-ylmethyl)-1H-pyrrolo[2,3-b]pyridin-6-yl)-1-oxoisoindolin-2-yl) piperidine-2,6-dione

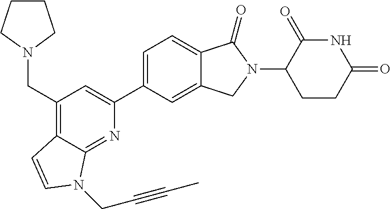 [SMILES: CC#CCn1ccc2c(CN3CCCC3)cc(-c3ccc4c(c3)CN(C3CCC(=O)NC3=O)C4=O)nc21]

The title compound was prepared in a manner analogous to Example 1 by reductive amination between of pyrrolidine and 1-(but-2-yn-1-yl)-6-chloro-1H-pyrrolo[2,3-b]pyridine-4-carbaldehyde (Intermediate 48) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₉H₂₉N₅O₃, 495.23; m/z found, 496.3 [M+H]⁺. ¹H NMR (400 MHZ, DMSO-d₆) δ 11.02 (s, 1H), 10.20 (s, 1H), 8.40 (s, 1H), 8.34 (d, J=8.0 Hz, 1H), 8.06 (s, 1H), 7.91 (d, J=8.0 Hz, 1H), 7.82 (d, J=3.6 Hz, 1H), 6.90 (d, J=3.6 Hz, 1H), 5.21 (s, 2H), 5.19-5.14 (m, 1H), 4.76 (d, J=5.0 Hz, 2H), 4.59 (d, J=17.4 Hz, 1H), 4.47 (d, J=17.4 Hz, 1H), 3.52 (s, 2H), 3.23 (s, 2H), 3.00-2.89 (m, 1H), 2.64 (d, J=16.6 Hz, 1H), 2.48-2.44 (m, 1H), 2.11-2.05 (m, 3H), 1.91-1.87 (m, 2H), 1.81 (s, 3H). ¹⁹F NMR (376 MHZ, DMSO-d₆) δ-73.56 (ppm).

### Example 149:2-(6-(2-(2,6-dioxopiperidin-3-yl)-1-oxoisoindolin-5-yl)-4-(pyrrolidin-1-ylmethyl)-1H-pyrrolo[2,3-b]pyridin-1-yl)-N,N-dimethylacetamide

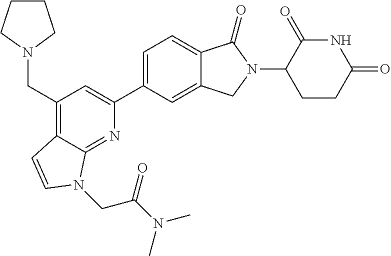 [SMILES: CN(C)C(=O)Cn1ccc2c(CN3CCCC3)cc(-c3ccc4c(c3)CN(C3CCC(=O)NC3=O)C4=O)nc21]

The title compound was prepared in a manner analogous to Example 1 by reductive amination between of pyrrolidine and 2-(6-chloro-4-formyl-1H-pyrrolo[2,3-b]pyridin-1-yl)-N,N-dimethylacetamide (Intermediate 49) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₉H₃₂N₆O₄, 528.3; m/z found, 529.3 [M+H]⁺.

¹H NMR (400 MHZ, DMSO-d₆) δ 11.02 (s, 1H), 8.37 (s, 1H), 8.33 (d, J=8.0 Hz, 1H), 8.17 (s, 1H), 8.02 (s, 1H), 7.84 (d, J=8.0 Hz, 1H), 7.54 (d, J=3.6 Hz, 1H), 6.74 (d, J=3.6 Hz, 1H), 5.30 (s, 2H), 5.18-5.13 (m, 1H), 4.57 (d, J=17.2 Hz, 1H)_{4.43} (d, J=17.2 Hz, 1H), 4.33 (s, 2H), 3.19 (s, 3H), 3.96-2.83 (m, 8H), 2.66-2.59 (m, 1H), 2.47-2.39 (m, 1H), 2.07-2.00 (m, 1H), 1.91-1.83 (m, 4H).

### Example 150:2-(6-(2-(2,6-dioxopiperidin-3-yl)-1-oxoisoindolin-5-yl)-4-(pyrrolidin-1-ylmethyl)-1H-pyrrolo[2,3-b]pyridin-1-yl)-N-methylacetamide

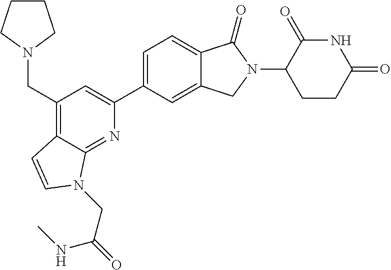 [SMILES: CNC(=O)Cn1ccc2c(CN3CCCC3)cc(-c3ccc4c(c3)CN(C3CCC(=O)NC3=O)C4=O)nc21]

The title compound was prepared in a manner analogous to Example 1 by reductive amination between of pyrrolidine and 2-(6-chloro-4-formyl-1H-pyrrolo[2,3-b]pyridin-1-yl)-N-methylacetamide (Intermediate 50) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calcd. for C₂₈H₃₀N₆O₄, 514.2; m/z found, 515.3 [M+H]⁺.

¹H NMR (400 MHZ, DMSO-d₆) δ 11.02 (s, 1H), 8.36 (s, 1H), 8.32 (d, J=8.0 Hz, 1H), 8.19 (d, J=4.8 Hz, 2H), 7.93 (s, 1H), 7.84 (d, J=8.0 Hz, 1H), 7.58 (d, J=3.6 Hz, 1H), 6.71 (d, J=3.6 Hz, 1H), 5.18-5.13 (m, 1H), 5.00 (s, 2H), 4.57 (d, J=17.2 Hz, 1H), 4.44 (d, J=17.2 Hz, 1H), 4.18 (s, 2H), 3.00-2.90 (m, 1H), 2.82-2.71 (m, 4H), 2.67-2.59 (m, 4H), 2.47-2.37 (m, 1H), 2.11-2.00 (m, 1H), 1.87-1.78 (m, 4H).

### Example 151:3-(5-(3-amino-7-(pyrrolidin-1-ylmethyl)-1H-pyrazolo[4,3-b]pyridin-5-yl)-1-oxoisoindolin-2-yl) piperidine-2,6-dione

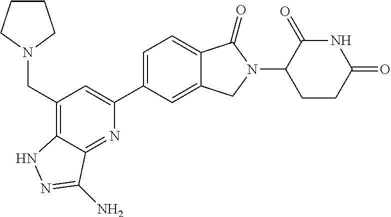 [SMILES: Nc1n[nH]c2c(CN3CCCC3)cc(-c3ccc4c(c3)CN(C3CCC(=O)NC3=O)C4=O)nc12]

The title compound was prepared in a manner analogous to Example 134 by reductive amination between of pyrrolidine and 3-amino-5-chloro-1-((2-(trimethylsilyl)ethoxy)methyl)-1H-pyrazolo[4,3-b]pyridine-7-carbaldehyde (Intermediate 27) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of the SEM group with TFA as a yellow solid. LC-MS (ESI): mass calced for: C₂₄H₂₅N₇O₃, 459.20; m/z found, 460.2 [M+H]⁺. ¹H NMR (400 MHz, DMSO-d₆) δ 12.13 (s, 1H), 11.02 (s, 1H), 10.08 (s, 1H), 8.37 (s, 1H), 8.32 (d, J=8.0 Hz, 1H), 8.19 (s, 1H), 7.90 (d, J=8.0 Hz, 1H), 5.19-5.14 (m, 1H), 4.66 (s, 2H), 4.58 (d, J=17.4 Hz, 1H), 4.45 (d, J=17.4 Hz, 1H), 3.25 (s, 4H), 2.98-2.89 (m, 1H), 2.64 (d, J=16.4 Hz, 1H), 2.47-2.38 (m, 1H), 2.08-2.04 (m, 3H), 1.93 (s, 2H). ¹⁹F NMR (400 MHZ, DMSO-d₆) δ -74.05 (ppm).

### Example 152:3-(5-(1-cyclopropyl-4-((3-(hydroxymethyl) azetidin-1-yl)methyl)-1H-pyrrolo[2,3-b]pyridin-6-yl)-1-oxoisoindolin-2-yl) piperidine-2,6-dione

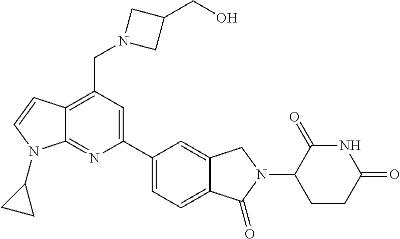 [SMILES: O=C1CCC(N2Cc3cc(-c4cc(CN5CC(CO)C5)c5ccn(C6CC6)c5n4)ccc3C2=O)C(=O)N1]

The title compound was prepared in a manner analogous to Example 1 by reductive amination between of azetidin-3-ylmethanol hydrochloride and 6-chloro-1-cyclopropyl-1H-pyrrolo[2,3-b]pyridine-4-carbaldehyde (Intermediate 30) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calced for: C₂₈H₂₉N₅O₄, 499.2; m/z found, 500.1 [M+H]⁺. ¹H NMR (400 MHZ, DMSO-d₆) δ 11.02 (s, 1H), 8.41 (s, 1H), 8.37 (d, J=8.0 Hz, 1H), 7.99 (s, 1H), 7.86 (d, J=8.0 Hz, 1H), 7.61 (d, J=3.4 Hz, 1H), 6.72 (s, 1H), 5.18-5.13 (m, 1H), 4.97 (s, 1H), 4.59 (d, J=17.2 Hz, 1H), 4.45 (d, J=17.2 Hz, 3H), 3.87 (s, 2H), 3.77-3.73 (m, 1H), 3.55 (s, 4H), 2.99-2.88 (m, 1H), 2.78 (s, 1H), 2.63 (d, J=17.0 Hz, 1H), 2.45 (dd, J=13.4, 4.8 Hz, 1H), 2.10-2.01 (m, 1H), 1.14-1.05 (m, 4H).

### Example 153:3-(5-(4-((3-isobutylpyrrolidin-1-yl)methyl)-1H-pyrrolo[2,3-b]pyridin-6-yl)-1-oxoisoindolin-2-yl) piperidine-2,6-dione

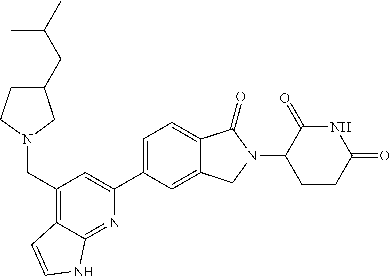 [SMILES: CC(C)CC1CCN(Cc2cc(-c3ccc4c(c3)CN(C3CCC(=O)NC3=O)C4=O)nc3[nH]ccc23)C1]

The title compound was prepared in a manner analogous to Example 1 by reductive amination between of 3-isobutylpyrrolidine (analog synthesis to Intermediate 46) and 6-chloro-1H-pyrrolo[2,3-b]pyridine-4-carbaldehyde (Intermediate 9) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₉H₃₃N₅O₃, 499.26; m/z found, 500.4 [M+H]⁺. ¹H NMR (400 MHZ, DMSO-d₆) δ 12.06 (s, 1H), 11.02 (s, 1H), 10.16 (s, 1H), 8.32 (s, 1H), 8.26 (d, J=8.2 Hz, 1H), 7.98 (d, J=4.8 Hz, 1H), 7.89 (d, J=8.0 Hz, 1H), 7.72-7.69 (m, 1H), 6.82 (dd, J=3.4, 1.8 Hz, 1H), 5.19-5.14 (m, 1H), 4.75 (d, J=5.2 Hz, 2H), 4.58 (d, J=17.4 Hz, 1H), 4.45 (d, J=17.4 Hz, 1H), 3.37-3.07 (m, 2H), 2.94-2.89 (m, 1H), 2.63 (d, J=15.6 Hz, 1H), 2.46-2.42 (m, 1H), 2.29 (dd, J=19.89, 10.4 Hz, 1H), 2.09-2.02 (m, 2H), 1.76-1.46 (m, 2H), 1.36-1.21 (m, 4H), 0.86 (t, J=5.8 Hz, 6H). 1° F. NMR (376 MHz, DMSO-d₆) δ-73.97 (ppm).

### Example 154:3-(5-(1-cyclopentyl-4-(pyrrolidin-1-ylmethyl)-1H-pyrrolo[2,3-b]pyridin-6-yl)-1-oxoisoindolin-2-yl) piperidine-2,6-dione

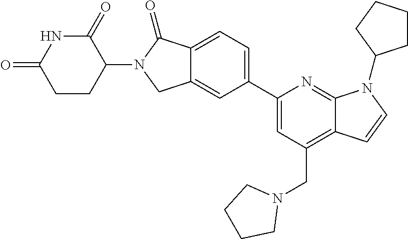 [SMILES: O=C1CCC(N2Cc3cc(-c4cc(CN5CCCC5)c5ccn(C6CCCC6)c5n4)ccc3C2=O)C(=O)N1]

The title compound was prepared in a manner analogous to Example 1 by reductive amination between of pyrrolidine and 6-chloro-1-cyclopentyl-1H-pyrrolo[2,3-b]pyridine-4-carbaldehyde (Intermediate 51) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₃₀H₃₃N₅O₃, 511.3; m/z found, 512.3 [M+H]⁺. ¹H NMR (400 MHZ, DMSO-d₆) δ 11.01 (s, 1H), 8.35 (s, 1H), 8.31 (d, J=9.2 Hz, 1H), 8.18 (s, 1H), 7.84 (d, J=8.0 Hz, 1H), 7.76 (s, 1H), 7.67 (d, J=3.6 Hz, 1H), 6.66 (d, J=3.6 Hz, 1H), 5.39-5.26 (m, 1H), 5.17-5.13 (m, 1H), 4.57 (d, J=17.2 Hz, 1H), 4.44 (d, J=17.2 Hz, 1H), 3.98 (s, 2H), 2.98-2.90 (m, 1H), 2.65-2.54 (m, 5H), 2.45-2.42 (m, 1H), 2.22-2.19 (m, 2H), 2.07-2.01 (m, 1H), 1.96-1.87 (m, 4H), 1.78-1.70 (m, 6H).

### Example 155:3-(5-(1-cyclopropyl-4-((3-(hydroxymethyl) pyrrolidin-1-yl)methyl)-1H-pyrrolo[2,3-b]pyridin-6-yl)-1-oxoisoindolin-2-yl) piperidine-2,6-dione

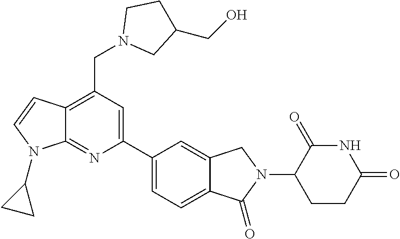 [SMILES: O=C1CCC(N2Cc3cc(-c4cc(CN5CCC(CO)C5)c5ccn(C6CC6)c5n4)ccc3C2=O)C(=O)N1]

The title compound was prepared in a manner analogous to Example 1 by reductive amination between of pyrrolidin-3-ylmethanol and 6-chloro-1-cyclopropyl-1H-pyrrolo[2,3-b]pyridine-4-carbaldehyde (Intermediate 30) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₉H₃₁N₅O₄, 513.24; m/z found, 514.2 [M+H]⁺. ¹H NMR (400 MHZ, DMSO-d₆) δ 11.02 (s, 1H), 8.39-8.32 (m, 2H), 8.14 (s, 1H), 7.91-7.83 (m, 2H), 7.55 (d, J=3.6 Hz, 1H), 6.66 (d, J=3.6 Hz, 1H), 5.18-5.13 (m, 1H), 4.58 (d, J=17.4 Hz, 1H), 4.45 (d, J=17.4 Hz, 1H), 4.15 (s, 2H), 3.77-3.71 (m, 1H), 3.35 (s, 2H), 2.98-2.90 (m, 1H), 2.80 (s, 2H), 2.63 (d, J=18.0 Hz, 2H), 2.45-2.38 (m, 1H), 2.32 (s, 1H), 2.12-1.81 (m, 3H), 1.51 (d, J=6.0 Hz, 1H), 1.11 (d, J=7.8 Hz, 4H).

### Example 156:3-(5-(7-(azetidin-1-ylmethyl)-1H-pyrrolo[3,2-b]pyridin-5-yl)-1-oxoisoindolin-2-yl) piperidine-2,6-dione

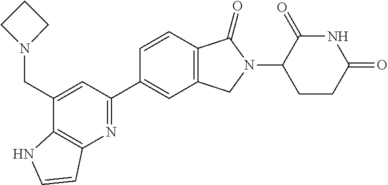 [SMILES: O=C1CCC(N2Cc3cc(-c4cc(CN5CCC5)c5[nH]ccc5n4)ccc3C2=O)C(=O)N1]

The title compound was prepared in a manner analogous to Example 134 by reductive amination between of azetidine and 5-chloro-1-((2-(trimethylsilyl)ethoxy)methyl)-1H-pyrrolo[3,2-b]pyridine-7-carbaldehyde (Intermediate 52) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of the SEM group with TFA as a yellow solid. LC-MS (ESI): mass calced for: C₂₄H₂₃N₅O₃, 429.18; m/z found, 430.2 [M+H]⁺. ¹H NMR (400 MHZ, DMSO-d₆) δ 11.89 (s, 1H), 11.02 (s, 1H), 10.34 (s, 1H), 8.35 (s, 1H), 8.25 (d, J=8.0 Hz, 1H), 7.94 (s, 1H), 7.92 (s, 1H), 7.87 (d, J=8.0 Hz, 1H), 6.76 (s, 1H), 5.16 (dd, J=13.2, 5.2 Hz, 1H), 4.77 (s, 2H), 4.57 (d, J=17.4 Hz, 1H), 4.44 (d, J=17.4 Hz, 1H), 4.22-4.05 (m, 4H), 3.01-2.88 (m, 1H), 2.63 (d, J=17.0 Hz, 1H), 2.45 (d, J=8.4 Hz, 3H), 2.12-2.00 (m, 1H). 1° F. NMR (376 MHz, DMSO) δ -74.28 (ppm).

### Example 157:3-(5-(4-((3-fluoropyrrolidin-1-yl)methyl)-1-(oxetan-3-yl)-1H-pyrrolo[2,3-b]pyridin-6-yl)-1-oxoisoindolin-2-yl) piperidine-2,6-dione

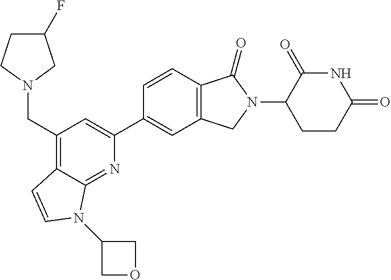 [SMILES: O=C1CCC(N2Cc3cc(-c4cc(CN5CCC(F)C5)c5ccn(C6COC6)c5n4)ccc3C2=O)C(=O)N1]

The title compound was prepared in a manner analogous to Example 1 by reductive amination between of 3-fluoropyrrolidine and 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₈H₂₈FN504, 517.56; m/z found, 518.2 [M+H]⁺. ¹H NMR (400 MHZ, DMSO-d₆) δ 11.01 (s, 1H), 8.38 (s, 1H), 8.33 (d, J=8.0 Hz, 1H), 8.14 (s, 1H), 7.99 (d, J=3.6 Hz, 1H), 7.84 (d, J=8.0 Hz, 1H), 7.80 (s, 1H), 6.79 (d, J=3.6 Hz, 1H), 6.15-6.11 (m, 1H), 5.30-5.13 (m, 2H), 5.07 (d, J=7.4 Hz, 4H), 4.57 (d, J=17.2 Hz, 1H), 4.44 (d, J=17.2 Hz, 1H), 3.98 (d, J=14.2 Hz, 2H), 2.99-2.82 (m, 3H), 2.80-2.68 (m, 1H), 2.63 (d, J=16.6 Hz, 1H), 2.48-2.39 (m, 2H), 2.23-2.15 (m, 1H), 2.08-2.00 (m, 1H), 1.99-1.82 (m, 1H). ¹⁹F NMR (400 MHZ, DMSO-d₆) δ-166.55 (ppm).

### Example 158:3-(5-(4-((3-hydroxypyrrolidin-1-yl)methyl)-1-(oxetan-3-yl)-1H-pyrrolo[2,3-b]pyridin-6-yl)-1-oxoisoindolin-2-yl) piperidine-2,6-dione

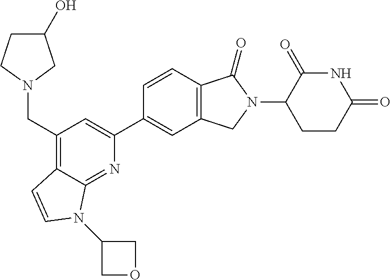 [SMILES: O=C1CCC(N2Cc3cc(-c4cc(CN5CCC(O)C5)c5ccn(C6COC6)c5n4)ccc3C2=O)C(=O)N1]

The title compound was prepared in a manner analogous to Example 1 by reductive amination between of pyrrolidin-3-ol and 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a black solid. LC-MS (ESI): mass calced for: C₂₈H₂₉N₅O₅, 515.57; m/z found, 516.3 [M+H]⁺. ¹H NMR (400 MHZ, DMSO-d₆) δ 11.01 (s, 1H), 8.38 (s, 1H), 8.33 (d, J=8.0 Hz, 1H), 8.15 (s, 1H), 7.99 (d, J=3.6 Hz, 1H), 7.84 (d, J=8.0 Hz, 1H), 7.81 (s, 1H), 6.79 (d, J=3.6 Hz, 1H), 6.14-6.10 (m, 1H), 5.18-5.13 (m, 1H), 5.11-5.03 (m, 4H), 4.57 (d, J=17.2 Hz, 1H), 4.44 (d, J=17.2 Hz, 1H), 4.27-4.21 (m, 1H), 3.98 (q, J=14.0 Hz, 2H), 2.98-2.89 (m, 1H), 2.81 (dd, J=9.6, 6.0 Hz, 1H), 2.73 (dd, J=15.6, 7.8 Hz, 1H), 2.63 (d, J=17.0 Hz, 1H), 2.56 (dd, J=14.0, 8.2 Hz, 1H), 2.48-2.40 (m, 2H), 2.09-1.99 (m, 2H), 1.60 (dt, J=12.8, 8.0 Hz, 1H).

### Example 159:3-(5-(1-cyclopropyl-4-((4-hydroxypiperidin-1-yl)methyl)-1H-pyrrolo[2,3-b]pyridin-6-yl)-1-oxoisoindolin-2-yl) piperidine-2,6-dione

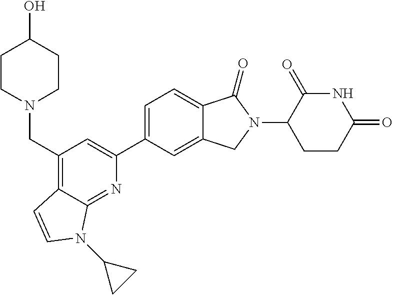 [SMILES: O=C1CCC(N2Cc3cc(-c4cc(CN5CCC(O)CC5)c5ccn(C6CC6)c5n4)ccc3C2=O)C(=O)N1]

The title compound was prepared in a manner analogous to Example 1 by reductive amination between of piperidin-4-ol and 6-chloro-1-cyclopropyl-1H-pyrrolo[2,3-b]pyridine-4-carbaldehyde (Intermediate 30) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₉H₃₁N₅O₄, 513.2; m/z found, 514.1 [M+H]⁺. ¹H NMR (400 MHZ, DMSO-d₆) δ 11.01 (s, 1H), 8.37 (s, 1H), 8.34 (d, J=8.2 Hz, 1H), 7.85 (d, J=8.0 Hz, 1H), 7.75 (s, 1H), 7.51 (s, 1H), 6.64 (s, 1H), 5.18-5.13 (m, 1H), 4.58 (d, J=17.2 Hz, 2H), 4.44 (d, J=17.2 Hz, 1H), 3.76 (dd, J=14.8, 8.0 Hz, 3H), 3.50 (d, J=13.8 Hz, 1H), 2.96-2.88 (m, 1H), 2.83-2.66 (m, 2H), 2.63 (d, J=15.8 Hz, 1H), 2.45-2.37 (m, 1H), 2.13-2.03 (m, 3H), 1.74 (s, 2H), 1.46 (s, 2H), 1.10 (d, J=7.2 Hz, 4H).

### Example 160:3-(5-(4-((3-hydroxyazetidin-1-yl)methyl)-1-(oxetan-3-yl)-1H-pyrrolo[2,3-b]pyridin-6-yl)-1-oxoisoindolin-2-yl) piperidine-2,6-dione

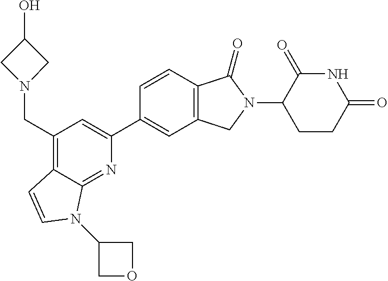 [SMILES: O=C1CCC(N2Cc3cc(-c4cc(CN5CC(O)C5)c5ccn(C6COC6)c5n4)ccc3C2=O)C(=O)N1]

The title compound was prepared in a manner analogous to Example 1 by reductive amination between of azetidin-3-ol and 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calced for: C₂₇H₂₇N₅O₅, 501.2; m/z found, 502.3 [M+H]⁺. ¹H NMR (400 MHZ, DMSO-d₆) δ 11.01 (s, 1H), 8.37 (s, 1H), 8.33 (d, J=8.2 Hz, 1H), 7.99 (d, J=3.6 Hz, 1H), 7.84 (d, J=8.0 Hz, 1H), 7.75 (s, 1H), 6.75 (d, J=3.6 Hz, 1H), 6.16-6.06 (m, 1H), 5.38 (s, 1H), 5.15 (dd, J=13.2, 5.2 Hz, 1H), 5.07 (d, J=7.6 Hz, 4H), 4.57 (d, J=17.2 Hz, 1H), 4.44 (d, J=17.2 Hz, 1H), 4.27 (d, J=6.0 Hz, 1H), 3.98 (s, 2H), 3.64 (s, 2H), 2.97-2.89 (m, 3H), 2.63 (d, J=16.2 Hz, 1H), 2.46-2.41 (m, 1H), 2.08-2.00 (m, 1H).

### Example 161:3-(5-(4-((3-fluoroazetidin-1-yl)methyl)-1-(oxetan-3-yl)-1H-pyrrolo[2,3-b]pyridin-6-yl)-1-oxoisoindolin-2-yl) piperidine-2,6-dione

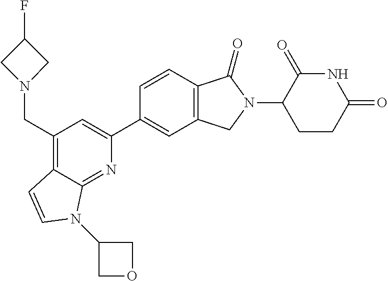 [SMILES: O=C1CCC(N2Cc3cc(-c4cc(CN5CC(F)C5)c5ccn(C6COC6)c5n4)ccc3C2=O)C(=O)N1]

The title compound was prepared in a manner analogous to Example 1 by reductive amination between of 3-fluoroazetidine hydrochloride and 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₇H₂₆FN504, 503.2; m/z found, 504.2 [M+H]⁺. ¹H NMR (400 MHZ, DMSO-d₆) δ 11.01 (s, 1H), 8.37 (s, 1H), 8.33 (d, J=8.0 Hz, 1H), 7.99 (d, J=3.6 Hz, 1H), 7.84 (d, J=8.0 Hz, 1H), 7.75 (s, 1H), 6.75 (d, J=3.6 Hz, 1H), 6.14-6.09 (m, 1H), 5.30-5.13 (m, 2H), 5.07 (d, J=7.2 Hz, 4H), 4.57 (d, J=17.2 Hz, 1H), 4.44 (d, J=17.2 Hz, 1H), 4.02 (s, 2H), 3.72-3.59 (m, 2H), 3.23 (dd, J=9.2, 4.6 Hz, 2H), 2.96-2.88 (m, 1H), 2.63 (d, J=16.2 Hz, 1H), 2.47-2.39 (m, 1H), 2.13-1.98 (m, 1H). ¹⁹F NMR (400 MHZ, DMSO-d₆) δ-177.65 (ppm).

### Example 162:3-(5-(4-((4-hydroxypiperidin-1-yl)methyl)-1-(oxetan-3-yl)-1H-pyrrolo[2,3-b]pyridin-6-yl)-1-oxoisoindolin-2-yl) piperidine-2,6-dione

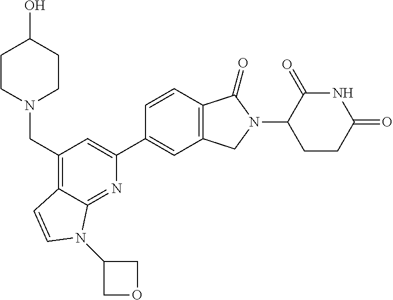 [SMILES: O=C1CCC(N2Cc3cc(-c4cc(CN5CCC(O)CC5)c5ccn(C6COC6)c5n4)ccc3C2=O)C(=O)N1]

The title compound was prepared in a manner analogous to Example 1 by reductive amination between of piperidin-4-ol and 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calced for: C₂₉H₃₁N₅O₅, 529.2; m/z found, 530.2 [M+H]⁺. ¹H NMR (400 MHZ, DMSO-d₆) δ 11.00 (s, 1H), 8.37 (s, 1H), 8.33 (d, J=8.0 Hz, 1H), 7.98 (d, J=3.6 Hz, 1H), 7.84 (d, J=8.0 Hz, 1H), 7.77 (s, 1H), 6.80 (d, J=3.6 Hz, 1H), 6.14-6.10 (m, 1H), 5.18-5.13 (m, 1H), 5.07 (d, J=7.2 Hz, 4H), 4.57 (d, J=17.2 Hz, 2H), 4.44 (d, J=17.2 Hz, 1H), 3.83 (s, 2H), 3.47 (s, 1H), 2.98-2.87 (m, 1H), 2.74 (s, 2H), 2.63 (d, J=16.8 Hz, 1H), 2.47-2.41 (m, 1H), 2.16 (s, 2H), 2.09-2.00 (m, 1H), 1.73 (d, J=9.0 Hz, 2H), 1.44 (d, J=9.0 Hz, 2H).

### Example 163:3-(5-(4-((4-(hydroxymethyl) piperidin-1-yl)methyl)-1-(oxetan-3-yl)-1H-pyrrolo[2,3-b]pyridin-6-yl)-1-oxoisoindolin-2-yl) piperidine-2,6-dione

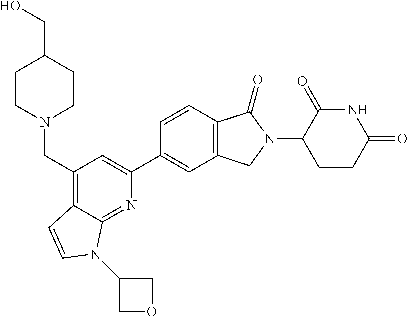 [SMILES: O=C1CCC(N2Cc3cc(-c4cc(CN5CCC(CO)CC5)c5ccn(C6COC6)c5n4)ccc3C2=O)C(=O)N1]

The title compound was prepared in a manner analogous to Example 1 by reductive amination between of piperidin-4-ylmethanol and 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calced for: C₃₀H₃₃N₅O₅, 543.2; m/z found, 544.2 [M+H]⁺. ¹H NMR (400 MHZ, DMSO-d₆) δ 11.00 (s, 1H), 8.37 (s, 1H), 8.32 (d, J=8.2 Hz, 1H), 7.98 (d, J=3.6 Hz, 1H), 7.84 (d, J=8.0 Hz, 1H), 7.77 (s, 1H), 6.80 (d, J=3.6 Hz, 1H), 6.15-6.09 (m, 1H), 5.19-5.13 (m, 1H), 5.07 (d, J=7.2 Hz, 4H), 4.57 (d, J=17.2 Hz, 1H), 4.42 (d, J=17.2 Hz, 2H), 3.83 (s, 2H), 3.27-3.24 (m, 2H), 2.96-2.85 (m, 3H), 2.63 (d, J=16.4 Hz, 1H), 2.47-2.41 (m, 1H), 2.09-2.02 (m, 3H), 1.64 (d, J=12.2 Hz, 2H), 1.34 (s, 1H), 1.19 (d, J=9.6 Hz, 2H).

### Example 164:3-(5-(4-((3-(hydroxymethyl) pyrrolidin-1-yl)methyl)-1-(oxetan-3-yl)-1H-pyrrolo[2,3-b]pyridin-6-yl)-1-oxoisoindolin-2-yl) piperidine-2,6-dione

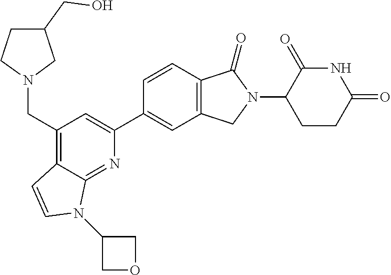 [SMILES: O=C1CCC(N2Cc3cc(-c4cc(CN5CCC(CO)C5)c5ccn(C6COC6)c5n4)ccc3C2=O)C(=O)N1]

The title compound was prepared in a manner analogous to Example 1 by reductive amination between of pyrrolidin-3-ylmethanol and 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calced for: C₂₉H₃₁N₅O₅, 529.2; m/z found, 530.2 [M+H]⁺. ¹H NMR (400 MHZ, DMSO-d₆) δ 11.01 (s, 1H), 8.37 (s, 1H), 8.33 (d, J=8.2 Hz, 1H), 7.98 (d, J=3.6 Hz, 1H), 7.84 (d, J=8.0 Hz, 1H), 7.80 (s, 1H), 6.78 (d, J=3.6 Hz, 1H), 6.19-6.10 (m, 1H), 5.17-5.13 (m, 1H), 5.07 (d, J=7.2 Hz, 4H), 4.58 (d, J=17.2 Hz, 1H), 4.44 (d, J=17.2 Hz, 1H), 3.95 (s, 2H), 3.28 (s, 2H), 2.96-2.88 (m, 1H), 2.65-2.60 (m, 3H), 2.45-2.37 (m, 2H), 2.24 (s, 1H), 2.06-2.03 (m, 1H), 1.84 (s, 1H), 1.42 (s, 1H), 1.25-1.21 (m, 1H).

### Example 165:3-(5-(4-((3-(hydroxymethyl) azetidin-1-yl)methyl)-1-(oxetan-3-yl)-1H-pyrrolo[2,3-b]pyridin-6-yl)-1-oxoisoindolin-2-yl) piperidine-2,6-dione

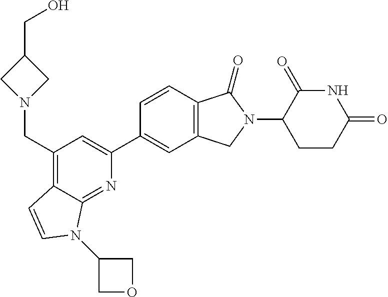 [SMILES: O=C1CCC(N2Cc3cc(-c4cc(CN5CC(CO)C5)c5ccn(C6COC6)c5n4)ccc3C2=O)C(=O)N1]

The title compound was prepared in a manner analogous to Example 1 by reductive amination between of azetidin-3-ylmethanol hydrochloride and 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calced for: C₂₈H₂₉N₅O₅, 515.2; m/z found, 516.2 [M+H]⁺. ¹H NMR (400 MHZ, DMSO-d₆) δ 11.01 (s, 1H), 8.37 (s, 1H), 8.32 (d, J=8.2 Hz, 1H), 7.98 (d, J=3.6 Hz, 1H), 7.84 (d, J=8.0 Hz, 1H), 7.78 (s, 1H), 6.76 (d, J=3.6 Hz, 1H), 6.17-6.09 (m, 1H), 5.17-5.13 (m, 1H), 5.07 (d, J=7.2 Hz, 4H), 4.57 (d, J=17.2 Hz, 2H), 4.44 (d, J=17.2 Hz, 1H), 3.94 (s, 2H), 3.52 (s, 2H), 3.36 (s, 2H), 3.03 (s, 1H), 2.98-2.89 (m, 1H), 2.65-2.60 (m, 2H), 2.45-2.37 (m, 1H), 2.06-2.03 (m, 1H).

### Example 166:3-(5-(1-cyclohexyl-4-(pyrrolidin-1-ylmethyl)-1H-pyrrolo[2,3-b]pyridin-6-yl)-1-oxoisoindolin-2-yl) piperidine-2,6-dione

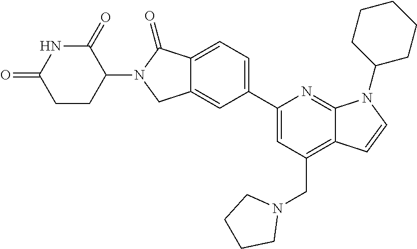 [SMILES: O=C1CCC(N2Cc3cc(-c4cc(CN5CCCC5)c5ccn(C6CCCCC6)c5n4)ccc3C2=O)C(=O)N1]

The title compound was prepared in a manner analogous to Example 1 by reductive amination between of pyrrolidine and 6-chloro-1-cyclohexyl-1H-pyrrolo[2,3-b]pyridine-4-carbaldehyde (Intermediate 53) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. For C₃₁H₃₅N₅O₃, 525.3; m/z found, 526.3 [M+H]⁺. ¹H NMR (400 MHZ, DMSO-d₆) δ 11.01 (s, 1H), 8.34 (s, 1H), 8.30 (d, J=8.0 Hz, 1H), 7.84 (d, J=8.0 Hz, 1H), 7.73 (s, 1H), 7.68 (d, J=3.2 Hz, 1H), 6.63 (d, J=3.2 Hz, 1H), 5.18-5.13 (m, 1H), 4.81 (t, J=11.2 Hz, 1H), 4.58 (d, J=17.2 Hz, 1H), 4.44 (d, J=17.2 Hz, 1H), 3.93 (s, 2H), 2.94 (t, J=12.8 Hz, 1H), 2.69-2.53 (m, 5H), 2.45-2.40 (m, 1H), 2.11-1.97 (m, 3H), 1.88 (d, J=10.6 Hz, 4H), 1.73 (s, 5H), 1.56-1.51 (m, 2H), 1.37-1.25 (m, 1H).

### Example 167:2-(1-((6-(2-(2,6-dioxopiperidin-3-yl)-1-oxoisoindolin-5-yl)-1H-pyrrolo[2,3-b]pyridin-4-yl)methyl) pyrrolidin-3-yl)-N,N-dimethylacetamide

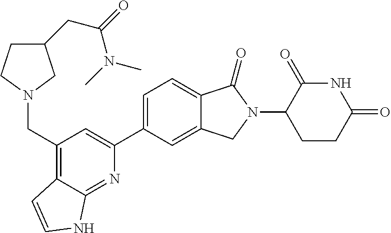 [SMILES: CN(C)C(=O)CC1CCN(Cc2cc(-c3ccc4c(c3)CN(C3CCC(=O)NC3=O)C4=O)nc3[nH]ccc23)C1]

The title compound was prepared in a manner analogous to Example 1 by reductive amination between N,N-dimethyl-2-(pyrrolidin-3-yl) acetamide hydrochloride (Intermediate 54) and 6-chloro-1H-pyrrolo[2,3-b]pyridine-4-carbaldehyde (Intermediate 9) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₉H₃₂N₆O₄, 528.61; m/z found, 529.6 [M+H]⁺. ¹H NMR (400 MHZ, DMSO-d₆) δ 11.76 (s, 1H), 11.01 (s, 1H), 8.31 (s, 1H), 8.25 (d, J=8.4 Hz, 1H), 8.15 (s, 1H), 7.83 (d, J=8.0 Hz, 1H), 7.73 (s, 1H), 7.52 (d, J=3.4 Hz, 1H), 6.63 (d, J=3.4 Hz, 1H), 5.18-5.13 (m, 1H), 4.56 (d, J=17.2 Hz, 1H), 4.43 (d, J=17.2 Hz, 1H), 4.02-3.93 (m, 2H), 2.98-2.90 (m, 4H), 2.83-2.78 (m, 1H), 2.76 (s, 3H), 2.64 (dd, J=16.2, 9.8 Hz, 3H), 2.45 (d, J=3.6 Hz, 1H), 2.41 (d, J=7.0 Hz, 3H), 2.28-2.23 (m, 1H), 2.07-1.97 (m, 2H), 1.46-1.37 (m, 1H).

### Example 168:3-(5-(1-(azetidin-3-yl)-4-(pyrrolidin-1-ylmethyl)-1H-pyrrolo[2,3-b]pyridin-6-yl)-1-oxoisoindolin-2-yl) piperidine-2,6-dione

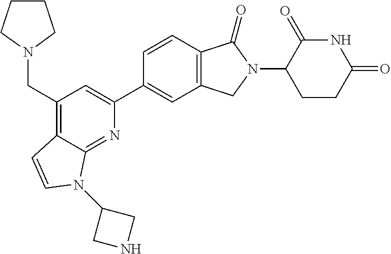 [SMILES: O=C1CCC(N2Cc3cc(-c4cc(CN5CCCC5)c5ccn(C6CNC6)c5n4)ccc3C2=O)C(=O)N1]

### Step A: tert-butyl 3-(6-chloro-4-(pyrrolidin-1-ylmethyl)-1H-pyrrolo[2,3-b]pyridin-1-yl) azetidine-1-carboxylate

To a solution of tert-butyl 3-(6-chloro-4-formyl-1H-pyrrolo[2,3-b]pyridin-1-yl) azetidine-1-carboxylate (Intermediate 55, 190 mg, 566 μmol, 1.0 eq) in DCM (5.00 mL) were added pyrrolidine (48.3 mg, 679 μmol, 1.2 eq) and AcOH (0.10 mL). The mixture was stirred at 30° C. for 16 h. Sodium triacetoxyborohydride (180 mg, 849 μmol, 1.5 eq) was added to above mixture and the resulting reaction mixture was stirred at 30° C. for 2 h. The reaction mixture was quenched with saturated aqueous NaHCO₃ solution (10 mL) and extracted with DCM (10 mL×3). The combined organic layers were washed with brine (15 mL), dried over anhydrous Na₂SO₄, filtered and concentrated under reduced pressure. The crude product was purified by Prep-TLC(DCM/MeOH=18/1 v/v) to afford tert-butyl 3-(6-chloro-4-(pyrrolidin-1-ylmethyl)-1H-pyrrolo[2,3-b]pyridin-1-yl) azetidine-1-carboxylate (70.0 mg, yield 32%) as a white solid. LC-MS (ESI): mass calced for: C₂₀H₂₇ClN₄O₂, 390.91; m/z found, 391.2 [M+H]⁺.

### Step B: tert-butyl 3-(6-(2-(2,6-dioxopiperidin-3-yl)-1-oxoisoindolin-5-yl)-4-(pyrrolidin-1-ylmethyl)-1H-pyrrolo[2,3-b]pyridin-1-yl) azetidine-1-carboxylate

To a solution of tert-butyl 3-(6-chloro-4-(pyrrolidin-1-ylmethyl)-1H-pyrrolo[2,3-b]pyridin-1-yl) azetidine-1-carboxylate (70.0 mg, 179 μmol, 1.0 eq) in 1,4-Dioxane (2.00 mL) and water (0.2 mL), were added 3-(1-oxo-5-(4,4,5,5-tetramethyl-1,3,2-dioxaborolan-2-yl) isoindolin-2-yl) piperidine-2,6-dione (Intermediate 13, 79.6 mg, 215 μmol, 1.2 eq), Potassium phosphate tribasic (114 mg, 537 μmol, 3.0 eq), and 1,1′-Bis(di-t-butylphosphino) ferrocene palladium dichloride (11.7 mg, 17.9 μmol, 0.1 eq). The mixture was stirred under N₂ at 95° C. for 8 h. After cooled to room temperature, the reaction mixture was filtered and the filtrate was concentrated in vacuum. The crude product was purified by Prep-TLC(DCM/MeOH=17/1 v/v) to obtain tert-butyl 3-(6-(2-(2,6-dioxopiperidin-3-yl)-1-oxoisoindolin-5-yl)-4-(pyrrolidin-1-ylmethyl)-1H-pyrrolo[2,3-b]pyridin-1-yl) azetidine-1-carboxylate (80.0 mg, yield 74%) as a yellow solid. LC-MS (ESI): mass calced for: C₃₃H₃₈N₆O₅, 598.7; m/z found, 599.2 [M+H]⁺.

### Step C: 3-(5-(1-(azetidin-3-yl)-4-(pyrrolidin-1-ylmethyl)-1H-pyrrolo[2,3-b]pyridin-6-yl)-1-oxoisoindolin-2-yl) piperidine-2,6-dione

To a solution of tert-butyl 3-(6-(2-(2,6-dioxopiperidin-3-yl)-1-oxoisoindolin-5-yl)-4-(pyrrolidin-1-ylmethyl)-1H-pyrrolo[2,3-b]pyridin-1-yl) azetidine-1-carboxylate (80.0 mg, 134 μmol, 1.0 eq) in DCM (3.00 mL) was added TFA (3.00 mL). The mixture was stirred at 30° C. for 30 min. After evaporation, the crude product was purified by Prep-HPLC with YMC-Actus Triart C18 (5 μm, 21.2×250 mm), and mobile phase of 5-25% ACN in water (0.05% TFA) over 10 min and 25-95% over 5 min then hold at 95% ACN for 3 min, at a flow rate of 20 mL/min to obtain 3-(5-(1-(azetidin-3-yl)-4-(pyrrolidin-1-ylmethyl)-1H-pyrrolo[2,3-b]pyridin-6-yl)-1-oxoisoindolin-2-yl) piperidine-2,6-dione (3.90 mg, yield 5%) as a white solid. LC-MS (ESI): mass calced for: C₂₈H₃₀N₆O₃, 498.59; m/z found, 499.3 [M+H]⁺. ¹H NMR (400 MHz, DMSO-d₆) δ 11.03 (s, 1H), 10.60 (s, 1H), 9.27 (s, 1H), 8.37 (d, J=10.4 Hz, 2H), 8.12 (s, 1H), 8.08 (d, J=3.6 Hz, 1H), 7.90 (d, J=8.0 Hz, 1H), 6.99 (d, J=3.6 Hz, 1H), 5.98-5.92 (m, 1H), 5.20-5.15 (m, 1H), 4.78 (s, 2H), 4.72 (s, 2H), 4.61-4.43 (m, 4H), 3.21 (s, 2H), 3.01-2.89 (m, 1H), 2.64 (d, J=17.4 Hz, 1H), 2.53 (s, 2H), 2.47-2.40 (m, 1H), 2.09-2.05 (m, 3H), 1.97-1.83 (m, 2H). ¹⁹F NMR (400 MHZ, DMSO-d₆) δ-73.68 (ppm).

### Example 169:3-(5-(1-(1-methylazetidin-3-yl)-4-(pyrrolidin-1-ylmethyl)-1H-pyrrolo[2,3-b]pyridin-6-yl)-1-oxoisoindolin-2-yl) piperidine-2,6-dione

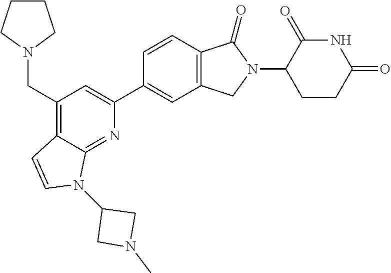 [SMILES: CN1CC(n2ccc3c(CN4CCCC4)cc(-c4ccc5c(c4)CN(C4CCC(=O)NC4=O)C5=O)nc32)C1]

### Step A: 1-(hydroxymethyl)-3-(5-(1-(1-methylazetidin-3-yl)-4-(pyrrolidin-1-ylmethyl)-1H-pyrrolo[2,3-b]pyridin-6-yl)-1-oxoisoindolin-2-yl) piperidine-2,6-dione

To a solution of 3-(5-(1-(azetidin-3-yl)-4-(pyrrolidin-1-ylmethyl)-1H-pyrrolo[2,3-b]pyridin-6-yl)-1-oxoisoindolin-2-yl) piperidine-2,6-dione (Example 168, 50.0 mg, 100 μmol, 1.0 eq) in DMF (1.00 mL) was added formaldehyde (30% W.t in H₂O) (1.00 mL, 333 μmol, 3.0 eq) and the mixture was stirred for 30 min. Sodium triacetoxyhydroborate (31.9 mg, 150 μmol, 1.5 eq) was added to above mixture and the resulting mixture was stirred at 30° C. for 2 h. After evaporation, the crude product was purified by Prep-HPLC with YMC-Actus Triart C18 (5 μm, 21.2×250 mm), and mobile phase of 5-35% ACN in water (0.1% FA) over 16 min and 35-95% ACN in water (0.1% FA) over 5 min then hold at 95% ACN for 3 min, at a flow rate of 20 mL/min to give a mixture of 1-(hydroxymethyl)-3-(5-(1-(1-methylazetidin-3-yl)-4-(pyrrolidin-1-ylmethyl)-1H-pyrrolo[2,3-b]pyridin-6-yl)-1-oxoisoindolin-2-yl) piperidine-2,6-dione and 3-(5-(1-(1-methylazetidin-3-yl)-4-(pyrrolidin-1-ylmethyl)-1H-pyrrolo[2,3-b]pyridin-6-yl)-1-oxoisoindolin-2-yl) piperidine-2,6-dione (20.0 mg, yield 36%) as a white solid. LC-MS (ESI): mass calced for: C₃₀H₃₄N₆O₄, 542.64; m/z found, 543.2 [M+H]⁺. ¹H NMR (400 MHZ, DMSO-d₆) δ 11.03 (s, 1H), 8.37 (s, 1H), 8.32 (d, J=7.8 Hz, 1H), 8.18 (s, 1H), 7.93 (s, 1H), 7.87 (d, J=12.2 Hz, 2H), 6.79 (d, J=2.7 Hz, 1H), 6.01 (d, J=140.6 Hz, 1H), 5.65 (s, 1H), 5.25 (dd, J=13.2, 4.6 Hz, 1H), 5.20-4.36 (m, 9H), 4.22-3.86 (m, 6H), 3.30 (d, J=9.2 Hz, 1H), 3.08 (t, J=12.8 Hz, 1H), 3.01-2.87 (m, 1H), 2.86-2.59 (m, 7H), 2.42 (d, J=9.0 Hz, 1H), 2.09 (d, J=12.0 Hz, 1H), 1.79 (s, 4H).

### Step B: 3-(5-(1-(1-methylazetidin-3-yl)-4-(pyrrolidin-1-ylmethyl)-1H-pyrrolo[2,3-b]pyridin-6-yl)-1-oxoisoindolin-2-yl) piperidine-2,6-dione

A solution of 1-(hydroxymethyl)-3-(5-(1-(1-methylazetidin-3-yl)-4-(pyrrolidin-1-ylmethyl)-1H-pyrrolo[2,3-b]pyridin-6-yl)-1-oxoisoindolin-2-yl) piperidine-2,6-dione (20.0 mg, 36.9 μmol, 1.0 eq) in ACN (2.00 mL) was adjusted to pH 8 with ammonia water at 0° C. and the resulting mixture was stirred at 5° C. for 30 min. After evaporation, the crude product was purified by Prep-HPLC with YMC-Actus Triart C18 (5 μm, 21.2×250 mm), and mobile phase of 5-30% ACN in water (0.1% FA) over 18 min and 30-95% ACN in water (0.1% FA) over 5 min then hold at 95% ACN for 3 min, at a flow rate of 20 mL/min to give 3-(5-(1-(1-methylazetidin-3-yl)-4-(pyrrolidin-1-ylmethyl)-1H-pyrrolo[2,3-b]pyridin-6-yl)-1-oxoisoindolin-2-yl) piperidine-2,6-dione formate (6.80 mg, yield 36%) as a white solid. LC-MS (ESI): mass calced for: C₂₉H₃₂N₆O₃, 512.61; m/z found, 513.3 [M+H]⁺. ¹H NMR (400 MHz, DMSO-d₆) δ 11.01 (s, 1H), 8.37 (s, 1H), 8.32 (d, J=8.0 Hz, 1H), 8.15 (s, 1H), 7.89 (d, J=3.6 Hz, 1H), 7.84 (d, J=8.0 Hz, 1H), 7.78 (s, 1H), 6.73 (d, J=3.6 Hz, 1H), 5.57-5.47 (m, 1H), 5.18-5.13 (m, 1H), 4.57 (d, J=17.2 Hz, 1H), 4.44 (d, J=17.2 Hz, 1H), 3.97 (s, 2H), 3.88 (t, J=7.2 Hz, 2H), 3.58 (d, J=7.2 Hz, 2H), 2.98-2.89 (m, 1H), 2.62 (dd, J=16.8, 2.2 Hz, 1H), 2.56 (s, 4H), 2.45 (s, 3H), 2.41 (dd, J=10.2, 3.4 Hz, 1H), 2.09-2.01 (m, 1H), 1.74 (s, 4H).

### Example 170:3-(1-oxo-5-(4-(pyrrolidin-1-ylmethyl)-1-(tetrahydrofuran-3-yl)-1H-pyrrolo[2,3-b]pyridin-6-yl) isoindolin-2-yl) piperidine-2,6-dione

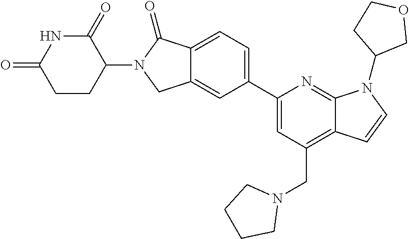 [SMILES: O=C1CCC(N2Cc3cc(-c4cc(CN5CCCC5)c5ccn(C6CCOC6)c5n4)ccc3C2=O)C(=O)N1]

The title compound was prepared in a manner analogous to Example 1 by reductive amination between pyrrolidine and 6-chloro-1-(tetrahydrofuran-3-yl)-1H-pyrrolo[2,3-b]pyridine-4-carbaldehyde (Intermediate 56) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₉H₃₁N₅O₄, 513.24; m/z found, 514.2 [M+H]⁺. ¹H NMR (400 MHZ, DMSO-d₆) δ 11.08 (s, 1H), 8.45 (s, 1H), 8.41 (d, J=8.2 Hz, 1H), 8.21 (s, 1H), 7.97 (s, 1H), 7.91 (d, J=8.0 Hz, 1H), 7.71 (d, J=3.6 Hz, 1H), 6.81 (d, J=3.6 Hz, 1H), 5.80-5.64 (m, 1H), 5.24-5.19 (m, 1H), 4.63 (d, J=17.2 Hz, 1H), 4.50 (d, J=17.2 Hz, 1H), 4.28-4.08 (m, 4H), 4.00-3.93 (m, 2H), 3.05-2.95 (m, 1H), 2.83 (s, 4H), 2.73-2.60 (m, 2H), 2.54-2.44 (m, 1H), 2.30-2.27 (m, 1H), 2.12-2.09 (m, 1H), 1.87 (s, 4H).

### Example 171:3-(1-oxo-5-(4-(pyrrolidin-1-ylmethyl)-1-(tetrahydro-2H-pyran-4-yl)-1H-pyrrolo[2,3-b]pyridin-6-yl) isoindolin-2-yl) piperidine-2,6-dione

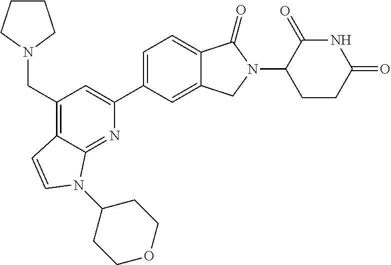 [SMILES: O=C1CCC(N2Cc3cc(-c4cc(CN5CCCC5)c5ccn(C6CCOCC6)c5n4)ccc3C2=O)C(=O)N1]

The title compound was prepared in a manner analogous to Example 1 by reductive amination between pyrrolidine and 6-chloro-1-(tetrahydro-2H-pyran-4-yl)-1H-pyrrolo[2,3-b]pyridine-4-carbaldehyde (Intermediate 57) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. For C₃₀H₃₃N₅O₄, 527.3; m/z found, 528.3 [M+H]⁺. ¹H NMR (400 MHZ, DMSO-d₆) δ 11.08 (s, 1H), 8.43 (s, 1H), 8.40 (d, J=8.0 Hz, 1H), 8.21 (s, 1H), 7.96 (s, 1H), 7.91 (d, J=8.0 Hz, 1H), 7.85 (d, J=3.6 Hz, 1H), 6.79 (d, J=3.6 Hz, 1H), 5.25-5.20 (m, 1H), 5.18-5.10 (m, 1H), 4.64 (d, J=17.2 Hz, 1H), 4.50 (d, J=17.2 Hz, 1H), 4.24 (s, 2H), 4.10 (dd, J=11.4, 3.6 Hz, 2H), 3.68 (t, J=11.2 Hz, 2H), 3.04-2.97 (m, 1H), 2.87-2.78 (m, 4H), 2.72-2.65 (m, 1H), 2.49 (dt, J=13.2, 8.0 Hz, 1H), 2.31-2.18 (m, 2H), 2.11 (dd, J=10.6, 5.2 Hz, 1H), 2.01 (dd, J=13.6, 4.4 Hz, 2H), 1.89-1.85 (m, 4H).

### Example 172:3-(5-(1-isopropyl-7-(pyrrolidin-1-ylmethyl)-1H-pyrrolo[3,2-b]pyridin-5-yl)-1-oxoisoindolin-2-yl) piperidine-2,6-dione

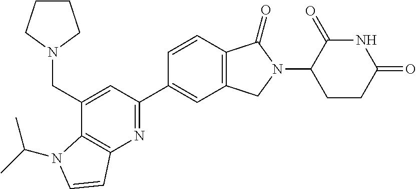 [SMILES: CC(C)n1ccc2nc(-c3ccc4c(c3)CN(C3CCC(=O)NC3=O)C4=O)cc(CN3CCCC3)c21]

The title compound was prepared in a manner analogous to Example 1 by reductive amination between pyrrolidine and 5-chloro-1-isopropyl-1H-pyrrolo[3,2-b]pyridine-7-carbaldehyde (Intermediate 58) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₈H₃₁N₅O₃, 485.24; m/z found, 486.3 [M+H]⁺. ¹H NMR (400 MHZ, DMSO-d₆) δ 11.01 (s, 1H), 8.35 (s, 1H), 8.27 (d, J=7.8 Hz, 2H), 7.88 (d, J=3.4 Hz, 1H), 7.81 (d, J=8.0 Hz, 1H), 7.73 (s, 1H), 6.71 (d, J=3.4 Hz, 1H), 5.36-5.24 (m, 1H), 5.18-5.13 (m, 1H), 4.55 (d, J=17.4 Hz, 1H), 4.43 (d, J=17.4 Hz, 1H), 4.00 (s, 2H), 3.27-3.20 (m, 4H), 2.94-2.90 (m, 1H), 2.63 (d, J=16.4 Hz, 1H), 2.45 (d, J=13.4 Hz, 1H), 2.09-2.01 (m, 1H), 1.72 (s, 4H), 1.49 (d, J=6.6 Hz, 6H).

### Example 173:3-(5-(1-(3-hydroxypropyl)-7-(pyrrolidin-1-ylmethyl)-1H-pyrrolo[3,2-b]pyridin-5-yl)-1-oxoisoindolin-2-yl) piperidine-2,6-dione

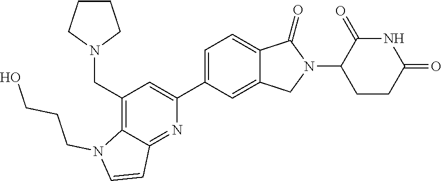 [SMILES: O=C1CCC(N2Cc3cc(-c4cc(CN5CCCC5)c5c(ccn5CCCO)n4)ccc3C2=O)C(=O)N1]

### Step A: 1-(3-((tert-butyldimethylsilyl)oxy)propyl)-5-chloro-7-(pyrrolidin-1-ylmethyl)-1H-pyrrolo[3,2-b]pyridine

To a solution of 1-(3-((tert-butyldimethylsilyl)oxy)propyl)-5-chloro-1H-pyrrolo[3,2-b]pyridine-7-carbaldehyde (Intermediate 59, 100 mg, 283 μmol, 1.0 eq) and pyrrolidine (30.2 mg, 425 mmol, 1.5 eq) in DCM (6.0 mL) was added AcOH (0.1 mL) and the reaction mixture was stirred at 40° C. for 2 h. Sodium triacetoxyborohydride (120 mg, 567 μmol, 2.0 eq) was added to above mixture and the resulting reaction mixture was stirred at 40° C. for 2 h. After evaporation, the residue was purified by Prep-TLC(DCM/MeOH=10/1 v/v) to obtain 1-(3-((tert-butyldimethylsilyl)oxy)propyl)-5-chloro-7-(pyrrolidin-1-ylmethyl)-1H-pyrrolo[3,2-b]pyridine (50 mg, yield 43%) as a yellow solid. LC-MS (ESI): mass calced for: C₂₁H₃₄ClN₃OSi, 407.22; m/z found, 408.2 [M+H]⁺. ¹H NMR (400 MHZ, CDCl₃) δ 7.24 (d, J=3.2 Hz, 1H), 6.97 (s, 1H), 6.57 (d, J=3.2 Hz, 1H), 4.46 (t, J=7.0 Hz, 2H), 3.85 (s, 2H), 3.52 (t, J=5.6 Hz, 2H), 2.52 (s, 4H), 1.94-1.81 (m, 2H), 1.74-1.70 (m, 4H), 0.85 (s, 9H),-0.00 (s, 6H).

### Step B: 3-(5-(1-(3-((tert-butyldimethylsilyl)oxy)propyl)-7-(pyrrolidin-1-ylmethyl)-1H-pyrrolo[3,2-b]pyridin-5-yl)-1-oxoisoindolin-2-yl) piperidine-2,6-dione

To a stirred mixture of 1-(3-((tert-butyldimethylsilyl)oxy)propyl)-5-chloro-7-(pyrrolidin-1-ylmethyl)-1H-pyrrolo[3,2-b]pyridine (50 mg, 123 μmol, 1.0 eq) in 1,4-Dioxane (5.0 mL) and H₂O (0.5 mL) were added 3-(1-oxo-5-(4,4,5,5-tetramethyl-1,3,2-dioxaborolan-2-yl) isoindolin-2-yl) piperidine-2,6-dione (54.4 mg, 147 μmol, 1.2 eq), Potassium phosphate tribasic (78 mg, 368 μmol, 3.0 eq), and 1,1′-Bis(di-t-butylphosphino) ferrocene palladium dichloride (8.0 mg, 12.3 μmol, 0.1 eq). The mixture was stirred under N₂ at 95° C. for 2 h. After cooled to room temperature, the reaction mixture was filtered and the filtrate was concentrated under reduced pressure. The residue was purified by Prep-TLC(MeOH/DCM=10/1 v/v) to give 3-(5-(1-(3-((tert-butyldimethylsilyl)oxy)propyl)-7-(pyrrolidin-1-ylmethyl)-1H-pyrrolo[3,2-b]pyridin-5-yl)-1-oxoisoindolin-2-yl) piperidine-2,6-dione (30.0 mg, yield 40%) as a brown solid. LC-MS (ESI): mass calced for: C₃₄H₄₅N₅₀₄Si, 615.32; m/z found, 616.2 [M+H]⁺.

### Step C: 3-(5-(1-(3-hydroxypropyl)-7-(pyrrolidin-1-ylmethyl)-1H-pyrrolo[3,2-b]pyridin-5-yl)-1-oxoisoindolin-2-yl) piperidine-2,6-dione

To a stirred mixture of 3-(5-(1-(3-((tert-butyldimethylsilyl)oxy)propyl)-7-(pyrrolidin-1-ylmethyl)-1H-pyrrolo[3,2-b]pyridin-5-yl)-1-oxoisoindolin-2-yl) piperidine-2,6-dione (30.0 mg, 48.7 μmol, 1.0 eq) in DCM (5.0 mL) was added TFA (1.0 mL). The resulting mixture was stirred at 25° C. for 8 h. After evaporation, the residue was purified by Prep-HPLC with YMC-Actus Triart 18C(5 μm, 20×250 mm), and mobile phase of 5-99% ACN in water (0.1% TFA) over 10 min and then hold at 100% ACN for 2 min, at a flow rate of 25 mL/min to obtain 3-(5-(1-(3-hydroxypropyl)-7-(pyrrolidin-1-ylmethyl)-1H-pyrrolo[3,2-b]pyridin-5-yl)-1-oxoisoindolin-2-yl) piperidine-2,6-dione trifluoroacetate (11.0 mg, yield 17%) as a white solid. LC-MS (ESI): mass calced for: C₂₈H₃₁N₅O₄, 501.24; m/z found, 502.3 [M+H]⁺. ¹H NMR (400 MHz, DMSO-d₆) δ 11.03 (s, 1H), 10.29 (s, 1H), 8.38 (s, 1H), 8.29 (d, J=8.0 Hz, 1H), 8.02 (s, 1H), 7.88 (d, J=8.0 Hz, 1H), 7.84 (d, J=3.4 Hz, 1H), 6.78 (d, J=3.4 Hz, 1H), 5.19-5.14 (m, 1H), 4.96 (s, 2H), 4.57 (d, J=17.2 Hz, 1H), 4.47-4.42 (m, 3H), 3.45 (s, 2H), 3.42 (t, J=5.6 Hz, 2H), 3.34 (s, 2H), 3.02-2.88 (m, 1H), 2.64 (d, J=16.8 Hz, 1H), 2.49-2.38 (m, 1H), 2.12-2.05 (m, 3H), 2.22-1.80 (m, 4H). ¹⁹F NMR (400 MHZ, DMSO-d₆) δ-74.35 (ppm).

### Example 174:3-(5-(1-cyclopropyl-7-(pyrrolidin-1-ylmethyl)-1H-pyrrolo[3,2-b]pyridin-5-yl)-1-oxoisoindolin-2-yl) piperidine-2,6-dione

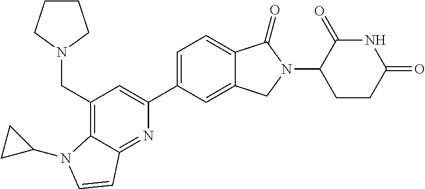 [SMILES: O=C1CCC(N2Cc3cc(-c4cc(CN5CCCC5)c5c(ccn5C5CC5)n4)ccc3C2=O)C(=O)N1]

The title compound was prepared in a manner analogous to Example 1 by reductive amination between pyrrolidine and 5-chloro-1-cyclopropyl-1H-pyrrolo[3,2-b]pyridine-7-carbaldehyde (Intermediate 60) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. For C₂₈H₂₉N₅O₃, 483.2; m/z found, 484.2 [M+H]⁺. ¹HNMR (400 MHZ, DMSO-d₆) δ 11.01 (s, 1H), 8.33 (s, 1H), 8.26-8.23 (m, 2H), 7.86-7.75 (m, 2H), 7.64 (d, J=3.6 Hz, 1H), 6.58 (d, J=3.2 Hz, 1H), 5.18-5.13 (m, 1H), 4.56 (d, J=17.2 Hz, 1H), 4.43 (d, J=17.2 Hz, 1H), 4.26 (s, 2H), 3.99 (d, J=4.0 Hz, 1H), 2.98-2.90 (m, 1H), 2.65-2.60 (m, 1H), 2.59-2.54 (m, 4H), 2.44 (dd, J=13.2, 4.8 Hz, 1H), 2.05 (dd, J=11.6, 6.4 Hz, 1H), 1.77-1.71 (m, 4H), 1.17-1.08 (m, 4H).

### Example 175:3-(5-(1-(2-hydroxyethyl)-7-(pyrrolidin-1-ylmethyl)-1H-pyrrolo[3,2-b]pyridin-5-yl)-1-oxoisoindolin-2-yl) piperidine-2,6-dione

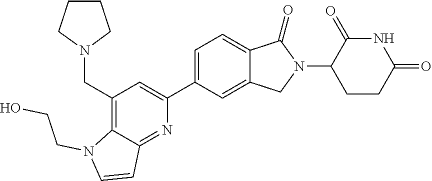 [SMILES: O=C1CCC(N2Cc3cc(-c4cc(CN5CCCC5)c5c(ccn5CCO)n4)ccc3C2=O)C(=O)N1]

The title compound was prepared in a manner analogous to Example 173 by reductive amination between pyrrolidine and 1-(2-((tert-butyldimethylsilyl)oxy)ethyl)-5-chloro-1H-pyrrolo[3,2-b]pyridine-7-carbaldehyde (Intermediate 61) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of OTBS group as a white solid. LC-MS (ESI): mass calced for: C₂₇H₂₉N₅O₄, 487.22; m/z found, 488.2 [M+H]⁺. ¹H NMR (400 MHZ, DMSO-d₆) δ 11.03 (s, 1H), 10.44 (s, 1H), 8.37 (s, 1H), 8.29 (d, J=8.0 Hz, 1H), 8.01 (s, 1H), 7.86 (dd, J=12.0, 5.6 Hz, 2H), 6.81 (d, J=3.6 Hz, 1H), 5.19-5.14 (m, 1H), 5.02 (s, 2H), 4.57 (d, J=17.2 Hz, 1H), 4.47 (dd, J=15.6, 11.0 Hz, 3H), 3.79 (s, 2H), 3.62 (s, 2H), 3.41-3.23 (m, 2H), 3.02-2.87 (m, 1H), 2.64 (d, J=16.9 Hz, 1H), 2.49-2.36 (m, 1H), 2.19-1.78 (m, 5H). 1° F. NMR (400 MHZ, DMSO-d₆) δ-74.13 (ppm).

### Example 176:3-(5-(1-(1,1-dioxidothietan-3-yl)-4-(pyrrolidin-1-ylmethyl)-1H-pyrrolo[2,3-b]pyridin-6-yl)-1-oxoisoindolin-2-yl) piperidine-2,6-dione

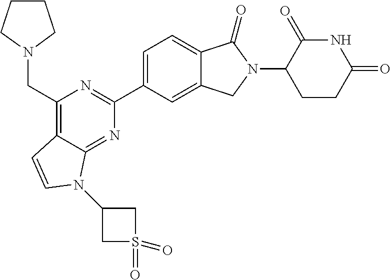 [SMILES: O=C1CCC(N2Cc3cc(-c4nc(CN5CCCC5)c5ccn(C6CS(=O)(=O)C6)c5n4)ccc3C2=O)C(=O)N1]

The title compound was prepared in a manner analogous to Example 1 by reductive amination between pyrrolidine and 6-chloro-1-(1,1-dioxidothietan-3-yl)-1H-pyrrolo[2,3-b]pyridine-4-carbaldehyde (Intermediate 35) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid (TFA salt). LC-MS (ESI): mass calced for: C₂₈H₂₉N₅O₅S, 547.63; m/z found, 548.3 [M+H]⁺. ¹H NMR (400 MHZ, DMSO-d₆) δ 11.02 (s, 1H), 10.17 (s, 1H), 8.48 (s, 1H), 8.38 (d, J=8.0 Hz, 1H), 8.13 (s, 1H), 7.99 (d, J=3.6 Hz, 1H), 7.90 (d, J=8.0 Hz, 1H), 6.96 (d, J=3.6 Hz, 1H), 5.86-5.80 (m, 1H), 5.17 (dd, J=13.2, 5.2 Hz, 1H), 4.94 (d, J=7.6 Hz, 4H), 4.77 (s, 2H), 4.58 (d, J=17.2 Hz, 1H), 4.45 (d, J=17.2 Hz, 1H), 3.52 (s, 2H), 3.22 (s, 2H), 3.00-2.89 (m, 1H), 2.63 (d, J=15.0 Hz, 1H), 2.44 (d, J=13.0 Hz, 1H), 2.09-2.04 (m, 3H), 1.95-1.84 (m, 2H). ¹⁹F NMR (400 MHZ, DMSO-d₆) δ-73.61 (ppm).

### Example 177:3-(5-(3-amino-1-isopropyl-7-(pyrrolidin-1-ylmethyl)-1H-pyrazolo[4,3-b]pyridin-5-yl)-1-oxoisoindolin-2-yl) piperidine-2,6-dione

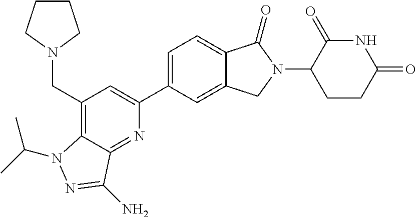 [SMILES: CC(C)n1nc(N)c2nc(-c3ccc4c(c3)CN(C3CCC(=O)NC3=O)C4=O)cc(CN3CCCC3)c21]

The title compound was prepared in a manner analogous to Example 1 by reductive amination between pyrrolidine and 3-amino-5-chloro-1-isopropyl-1H-pyrazolo[4,3-b]pyridine-7-carbaldehyde (Intermediate 62) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₇H₃₁N₇₀₃Si, 501.25; m/z found, 502.3 [M+H]⁺. ¹H NMR (400 MHZ, DMSO-d₆) δ 11.01 (s, 1H), 8.36 (s, 1H), 8.33 (d, J=8.0 Hz, 1H), 8.14 (s, 1H), 7.88 (s, 1H), 7.82 (d, J=8.0 Hz, 1H), 5.54 (s, 2H), 5.17-5.13 (m, 1H), 5.12-5.04 (m, 1H), 4.55 (d, J=17.2 Hz, 1H), 4.42 (d, J=17.2 Hz, 1H), 3.96 (s, 2H), 2.98-2.89 (m, 1H), 2.62 (d, J=15.6 Hz, 1H), 2.51 (s, 4H), 2.44 (s, 1H), 2.09-2.00 (m, 1H), 1.72 (s, 4H), 1.40 (d, J=6.4 Hz, 6H).

### Example 178:3-(5-(3-amino-1-ethyl-7-(pyrrolidin-1-ylmethyl)-1H-pyrazolo[4,3-b]pyridin-5-yl)-1-oxoisoindolin-2-yl) piperidine-2,6-dione

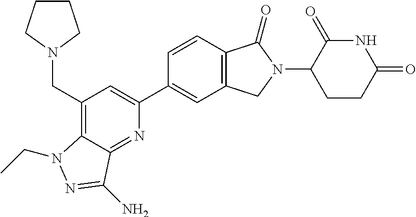 [SMILES: CCn1nc(N)c2nc(-c3ccc4c(c3)CN(C3CCC(=O)NC3=O)C4=O)cc(CN3CCCC3)c21]

The title compound was prepared in a manner analogous to Example 1 by reductive amination between pyrrolidine and 3-amino-5-chloro-1-ethyl-1H-pyrazolo[4,3-b]pyridine-7-carbaldehyde (Intermediate 63) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calcd. for C₂₆H₂₉N₇O₃, 487.56; m/z found, 488.3 [M+H]⁺. ¹H NMR (400 MHZ, DMSO-d₆) δ 11.01 (s, 1H), 8.37 (s, 1H), 8.33 (d, J=8.4 Hz, 1H), 8.17 (s, 1H), 7.90 (s, 1H), 7.82 (d, J=8.0 Hz, 1H), 5.53 (s, 2H), 5.17-5.14 (m, 1H), 4.55 (d, J=17.2 Hz, 1H), 4.46-4.33 (m, 3H), 3.94 (s, 2H), 2.93-2.90 (m, 1H), 2.67-2.60 (m, 5H), 2.46-2.41 (m, 1H), 2.08-2.01 (m, 1H), 1.73 (s, 4H), 1.32 (t, J=7.2 Hz, 3H).

### Example 179:3-(5-(3-(dimethylamino)-7-(pyrrolidin-1-ylmethyl)-1H-pyrazolo[4,3-b]pyridin-5-yl)-1-oxoisoindolin-2-yl) piperidine-2,6-dione

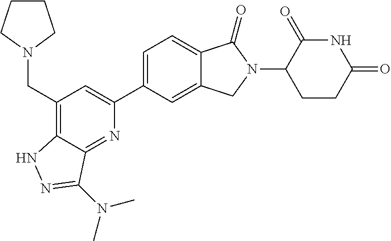 [SMILES: CN(C)c1n[nH]c2c(CN3CCCC3)cc(-c3ccc4c(c3)CN(C3CCC(=O)NC3=O)C4=O)nc12]

The title compound was prepared in a manner analogous to Example 173 by reductive amination between pyrrolidine and 5-chloro-3-(dimethylamino)-1-((2-(trimethylsilyl)ethoxy)methyl)-1H-pyrazolo[4,3-b]pyridine-7-carbaldehyde (Intermediate 64) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of N-SEM group as a yellow solid. LC-MS (ESI): mass calced for: C₂₆H₂₉N₇O₃, 487.1; m/z found, 488.1 [M+H]⁺. ¹H NMR (400 MHz, DMSO-d₆) δ 12.40 (s, 1H), 11.02 (s, 1H), 10.04 (s, 1H), 8.32 (s, 1H), 8.27 (d, J=8.0 Hz, 1H), 8.22 (s, 1H), 7.90 (d, J=8.0 Hz, 1H), 5.19-5.14 (m, 1H), 4.68 (s, 2H), 4.59 (d, J=17.4 Hz, 1H), 4.45 (d, J=17.4 Hz, 1H), 3.55 (s, 2H), 3.26 (s, 6H), 2.97-2.88 (m, 1H), 2.61 (s, 1H), 2.54 (s, 2H), 2.43 (s, 1H), 2.07 (s, 3H), 1.91 (s, 2H).

### Example 180:3-(5-(3-methyl-7-(pyrrolidin-1-ylmethyl)-3H-imidazo[4,5-b]pyridin-5-yl)-1-oxoisoindolin-2-yl) piperidine-2,6-dione

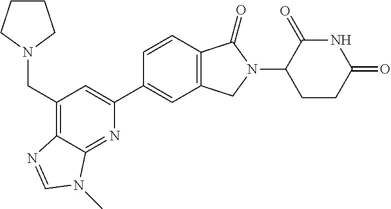 [SMILES: Cn1cnc2c(CN3CCCC3)cc(-c3ccc4c(c3)CN(C3CCC(=O)NC3=O)C4=O)nc21]

The title compound was prepared in a manner analogous to Example 88 by displacement between pyrrolidine and 7-(bromomethyl)-5-chloro-3-methyl-3H-imidazo[4,5-b]pyridine (Intermediate 65) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₅H₂₆N₆O₃, 458.52; m/z found, 459.3 [M+H]⁺.

¹H NMR (400 MHZ, DMSO-d₆) δ 11.03 (s, 1H), 8.52 (s, 1H), 8.39 (s, 1H), 8.33 (d, J=8.0 Hz, 1H), 8.15 (s, 1H), 8.10 (s, 1H), 7.88 (d, J=8.0 Hz, 1H), 5.19-5.14 (m, 1H), 4.58 (d, J=17.4 Hz, 1H), 4.46 (d, J=17.4 Hz, 1H), 4.37 (s, 2H), 3.94 (s, 3H), 2.99-2.91 (m, 1H), 2.88 (s, 4H), 2.63 (d, J=16.0 Hz, 1H), 2.47-2.42 (m, 1H), 2.10-2.02 (m, 1H), 1.83 (s, 4H).

### Example 181:3-(5-(3-(methylamino)-7-(pyrrolidin-1-ylmethyl)-1H-pyrazolo[4,3-b]pyridin-5-yl)-1-oxoisoindolin-2-yl) piperidine-2,6-dione

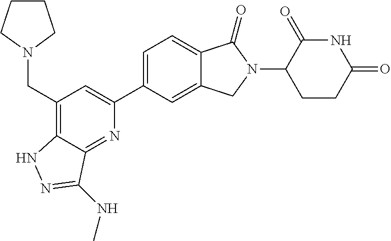 [SMILES: CNc1n[nH]c2c(CN3CCCC3)cc(-c3ccc4c(c3)CN(C3CCC(=O)NC3=O)C4=O)nc12]

The title compound was prepared in a manner analogous to Example 173 by reductive amination between pyrrolidine and 5-chloro-3-(methylamino)-1-((2-(trimethylsilyl)ethoxy)methyl)-1H-pyrazolo[4,3-b]pyridine-7-carbaldehyde (Intermediate 66) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of N-SEM group as a yellow solid (TFA salt). LC-MS (ESI): mass calced for: C₂₅H₂₇N₇O₃, 473.22; m/z found, 474.3 [M+H]⁺. ¹H NMR (400 MHZ, DMSO-d₆) δ 12.08 (s, 1H), 11.02 (s, 1H), 10.12 (s, 1H), 8.34 (s, 1H), 8.30 (d, J=8.0 Hz, 1H), 8.19 (s, 1H), 7.89 (d, J=8.0 Hz, 1H), 5.17-5.13 (m, 1H), 4.66 (s, 2H), 4.57 (d, J=17.4 Hz, 1H), 4.45 (d, J=17.4 Hz, 1H), 3.41-3.25 (m, 4H), 2.95 (s, 3H), 2.90 (dd, J=8.0, 4.0 Hz, 1H), 2.63 (d, J=18.0 Hz, 1H), 2.46-2.42 (m, 1H), 2.07 (s, 3H), 1.92 (s, 2H). 1° F. NMR (400 MHZ, DMSO-d₆) δ-74.01 (ppm).

### Example 182:3-(1-oxo-5-(4-(pyrrolidin-1-ylmethyl)-7H-pyrrolo[2,3-d]pyrimidin-2-yl) isoindolin-2-yl) piperidine-2,6-dione

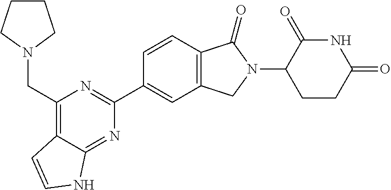 [SMILES: O=C1CCC(N2Cc3cc(-c4nc(CN5CCCC5)c5cc[nH]c5n4)ccc3C2=O)C(=O)N1]

The title compound was prepared in a manner analogous to Example 1 by Suzuki coupling of 2-chloro-4-(pyrrolidin-1-ylmethyl)-7H-pyrrolo[2,3-d]pyrimidine (Intermediate 78) with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₄H₂₄N₆O₃,444.50; m/z found, 445.2 [M+H]⁺. ¹H NMR (400 MHZ, DMSO-d₆) δ 12.16 (s, 1H), 11.02 (s, 1H), 8.62 (d, J=12.0 Hz, 2H), 8.18 (s, 1H), 7.86 (d, J=8.0 Hz, 1H), 7.57 (s, 1H), 6.80 (s, 1H), 5.18 5.14 (m, 1H), 4.59 (d, J=17.2 Hz, 1H), 4.46 (d, J=17.2 Hz, 1H), 4.10 (s, 2H), 2.97-2.90 (m, 1H), 2.65 (s, 5H), 2.45-2.41 (m, 1H), 2.07-2.02 (m, 1H), 1.75 (s, 4H).

### Example 183:3-(5-(3-amino-1-methyl-7-(pyrrolidin-1-ylmethyl)-1H-pyrazolo[4,3-b]pyridin-5-yl)-1-oxoisoindolin-2-yl) piperidine-2,6-dione

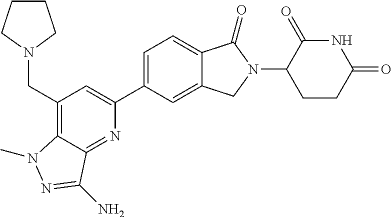 [SMILES: Cn1nc(N)c2nc(-c3ccc4c(c3)CN(C3CCC(=O)NC3=O)C4=O)cc(CN3CCCC3)c21]

The title compound was prepared in a manner analogous to Example 1 by reductive amination between pyrrolidine and 3-amino-5-chloro-1-methyl-1H-pyrazolo[4,3-b]pyridine-7-carbaldehyde (Intermediate 67) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₅H₂₇N₇O₃, 473.22; m/z found, 474.3 [M+H]⁺. ¹H NMR (400 MHZ, DMSO-d₆) δ 11.02 (s, 1H), 8.37 (s, 1H), 8.33 (d, J=8.2 Hz, 1H), 7.88 (s, 1H), 7.83 (d, J=8.0 Hz, 1H), 5.50 (s, 2H), 5.15 (dd, J=13.2, 5.2 Hz, 1H), 4.56 (d, J=17.2 Hz, 1H), 4.43 (d, J=17.2 Hz, 1H), 4.03 (s, 3H), 3.98 (s, 2H), 2.98-2.89 (m, 1H), 2.63 (d, J=17.4 Hz, 1H), 2.54 (s, 4H), 2.45 (dd, J=8.8, 4.8 Hz, 1H), 2.09-2.01 (m, 1H), 1.74 (s, 4H).

### Example 184:3-(5-(7-methyl-4-(pyrrolidin-1-ylmethyl)-7H-pyrrolo[2,3-d]pyrimidin-2-yl)-1-oxoisoindolin-2-yl) piperidine-2,6-dione

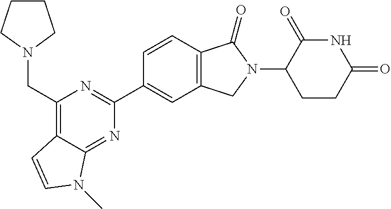 [SMILES: Cn1ccc2c(CN3CCCC3)nc(-c3ccc4c(c3)CN(C3CCC(=O)NC3=O)C4=O)nc21]

The title compound was prepared in a manner analogous to Example 1 by reductive amination between pyrrolidine and 2-chloro-7-methyl-7H-pyrrolo[2,3-d]pyrimidine-4-carbaldehyde (Intermediate 68) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₅H₂₆N₆O₃, 458.52; m/z found, 459.2 [M+H]⁺. ¹HNMR (400 MHZ, DMSO-d₆) δ 11.04 (s, 1H), 8.74 (d, J=11.8 Hz, 2H), 7.89 (d, J=8.0 Hz, 1H), 7.67 (s, 1H), 6.82 (d, J=3.4 Hz, 1H), 5.19-5.14 (m, 1H), 4.61 (d, J=17.4 Hz, 1H), 4.49 (s, 2H), 4.47 (d, J=17.2 Hz, 1H), 3.93 (s, 3H), 3.17-2.88 (m, 5H), 2.64 (d, J=16.8 Hz, 1H), 2.44-2.38 (m, 1H), 2.09-2.02 (m, 1H), 1.87 (s, 4H).

### Example 185:3-(5-(4-((4-(methylsulfonyl) piperidin-1-yl)methyl)-1-(oxetan-3-yl)-1H-pyrrolo[2,3-b]pyridin-6-yl)-1-oxoisoindolin-2-yl) piperidine-2,6-dione

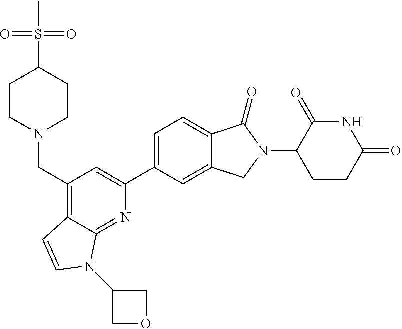 [SMILES: CS(=O)(=O)C1CCN(Cc2cc(-c3ccc4c(c3)CN(C3CCC(=O)NC3=O)C4=O)nc3c2ccn3C2COC2)CC1]

The title compound was prepared in a manner analogous to Example 1 by reductive amination between 4-(methylsulfonyl) piperidine and 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a gray solid. LC-MS (ESI): mass calced for: C₃₀H₃₃N₅₀₆S, 591.22; m/z found, 592.3 [M+H]⁺. ¹H NMR (400 MHZ, DMSO-d₆) δ 11.02 (s, 1H), 8.38 (s, 1H), 8.34 (d, J=8.5 Hz, 1H), 8.21 (s, 1H), 8.00 (d, J=3.6 Hz, 1H), 7.85 (d, J=8.0 Hz, 1H), 7.78 (s, 1H), 6.82 (d, J=3.6 Hz, 1H), 6.17-6.09 (m, 1H), 5.17-5.13 (m, 1H), 5.08 (d, J=7.2 Hz, 4H), 4.58 (d, J=17.4 Hz, 1H), 4.45 (d, J=17.4 Hz, 1H), 3.88 (s, 2H), 3.11-3.01 (m, 3H), 2.93 (d, J=9.4 Hz, 4H), 2.67-2.60 (m, 1H), 2.47-2.40 (m, 1H), 2.13-1.97 (m, 5H), 1.71-1.62 (m, 2H).

### Example 186:3-(5-(1-(oxetan-3-yl)-4-((tetrahydro-1H-furo[3,4-c]pyrrol-5 (3H)-yl)methyl)-1H-pyrrolo[2,3-b]pyridin-6-yl)-1-oxoisoindolin-2-yl) piperidine-2,6-dione

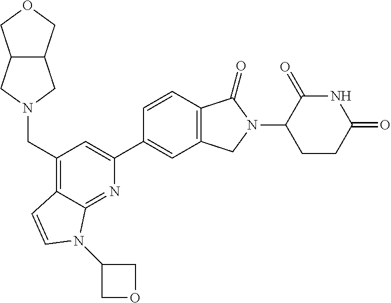 [SMILES: O=C1CCC(N2Cc3cc(-c4cc(CN5CC6COCC6C5)c5ccn(C6COC6)c5n4)ccc3C2=O)C(=O)N1]

The title compound was prepared in a manner analogous to Example 1 by reductive amination between hexahydro-1H-furo[3,4-c]pyrrole HCl salt and 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₃₀H₃₁N₅O₅, 541.61; m/z found, 542.3 [M+H]⁺. ¹H NMR (400 MHZ, DMSO-d₆) δ 11.07 (s, 1H), 8.43 (s, 1H), 8.39 (d, J=8.0 Hz, 1H), 8.20 (s, 1H), 8.05 (d, J=3.6 Hz, 1H), 7.90 (d, J=8.0 Hz, 1H), 7.85 (s, 1H), 6.87 (d, J=3.6 Hz, 1H), 6.20-6.16 (m, 1H), 5.24-5.20 (m, 1H), 5.13 (d, J=7.2 Hz, 4H), 4.63 (d, J=17.2 Hz, 1H), 4.50 (d, J=17.2 Hz, 1H), 4.02 (s, 2H), 3.83-3.76 (m, 2H), 3.49-3.46 (m, 2H), 3.05-2.95 (m, 1H), 2.79 (s, 2H), 2.69 (d, J=13.4 Hz, 3H), 2.51 (s, 3H), 2.15-2.07 (m, 1H).

### Example 187:3-(5-(4-((4-fluoropiperidin-1-yl)methyl)-1-(oxetan-3-yl)-1H-pyrrolo[2,3-b]pyridin-6-yl)-1-oxoisoindolin-2-yl) piperidine-2,6-dione

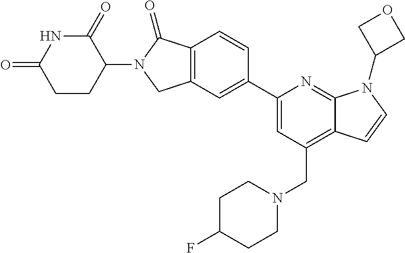 [SMILES: O=C1CCC(N2Cc3cc(-c4cc(CN5CCC(F)CC5)c5ccn(C6COC6)c5n4)ccc3C2=O)C(=O)N1]

The title compound was prepared in a manner analogous to Example 1 by reductive amination between 4-fluoropiperidine and 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₉H₃₀FN504, 531.23; m/z found, 532.2 [M+H]⁺. ¹H NMR (400 MHZ, DMSO-d₆) δ 11.08 (s, 1H), 8.45 (s, 1H), 8.40 (d, J=8.4 Hz, 1H), 8.21 (s, 1H), 8.05 (d, J=3.6 Hz, 1H), 7.91 (d, J=8.0 Hz, 1H), 7.85 (s, 1H), 6.88 (d, J=3.6 Hz, 1H), 6.23-6.14 (m, 1H), 5.24-5.19 (m, 1H), 5.14 (d, J=7.2 Hz, 4H), 4.78-4.65 (m, 1H), 4.64 (d, J=17.2 Hz, 1H), 4.51 (d, J=17.2 Hz, 1H), 3.93 (s, 2H), 3.03-2.97 (m, 1H), 2.71-2.67 (m, 3H), 2.51-2.45 (m, 3H), 2.11 (t, J=11.4 Hz, 1H), 2.01-1.90 (m, 2H), 1.81-1.77 (m, 2H). 19FNMR (400 MHZ, DMSO-d₆) δ-73.45 (ppm).

### Example 188:3-(5-(4-((2-oxa-7-azaspiro[3.5]nonan-7-yl)methyl)-1-(oxetan-3-yl)-1H-pyrrolo[2,3-b]pyridin-6-yl)-1-oxoisoindolin-2-yl) piperidine-2,6-dione

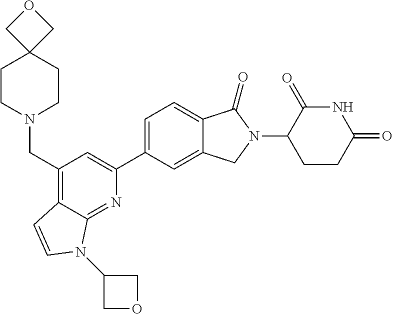 [SMILES: O=C1CCC(N2Cc3cc(-c4cc(CN5CCC6(CC5)COC6)c5ccn(C6COC6)c5n4)ccc3C2=O)C(=O)N1]

The title compound was prepared in a manner analogous to Example 1 by reductive amination between 2-oxa-7-azaspiro[3.5]nonane and 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₃₁H₃₃N₅O₅, 555.63; m/z found, 556.2 [M+H]⁺. ¹H NMR (400 MHZ, DMSO-d₆) δ 11.07 (s, 1H), 8.43 (s, 1H), 8.38 (d, J=8.0 Hz, 1H), 8.25 (s, 1H), 8.03 (d, J=3.4 Hz, 1H), 7.89 (d, J=8.0 Hz, 1H), 7.82 (s, 1H), 6.85 (d, J=3.4 Hz, 1H), 6.21-6.14 (m, 1H), 5.21-5.16 (m, 1H), 5.13 (d, J=7.2 Hz, 4H), 4.62 (d, J=17.2 Hz, 1H), 4.50 (d, J=17.2 Hz, 1H), 4.33 (s, 4H), 3.85 (s, 3H), 3.04-2.94 (m, 1H), 2.69 (d, J=17.0 Hz, 1H), 2.53-2.46 (m, 1H), 2.41 (s, 4H), 2.15-2.07 (m, 1H), 1.86 (s, 4H).

### Example 189:3-(5-(4-((4-methoxypiperidin-1-yl)methyl)-1-(oxetan-3-yl)-1H-pyrrolo[2,3-b]pyridin-6-yl)-1-oxoisoindolin-2-yl) piperidine-2,6-dione

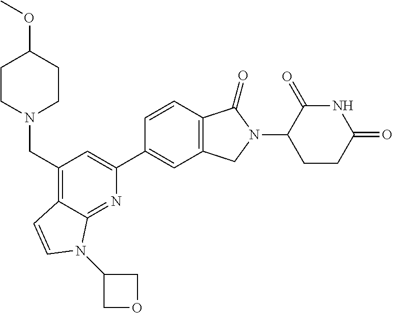 [SMILES: COC1CCN(Cc2cc(-c3ccc4c(c3)CN(C3CCC(=O)NC3=O)C4=O)nc3c2ccn3C2COC2)CC1]

The title compound was prepared in a manner analogous to Example 1 by reductive amination between 4-methoxypiperidine and 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calcd. for C₃₀H₃₃N₅O₅, 543.25; m/z found, 544.3 [M+H]⁺. ¹H NMR (400 MHZ, DMSO-d₆) δ 11.01 (s, 1H), 8.37 (s, 1H), 8.33 (d, J=8.4 Hz, 1H), 8.24 (s, 1H), 7.98 (d, J=3.6 Hz, 1H), 7.84 (d, J=8.0 Hz, 1H), 7.77 (s, 1H), 6.80 (d, J=3.6 Hz, 1H), 6.14-6.10 (m, 1H), 5.17-5.13 (m, 1H), 5.07 (d, J=7.2 Hz, 4H), 4.57 (d, J=17.4 Hz, 1H), 4.44 (d, J=17.4 Hz, 1H), 3.83 (s, 2H), 3.22 (s, 3H), 3.18 (s, 1H), 2.99-2.89 (m, 1H), 2.71 (s, 2H), 2.63 (d, J=17.8 Hz, 1H), 2.47-2.40 (m, 1H), 2.20 (t, J=9.6 Hz, 2H), 2.10-2.00 (m, 1H), 1.83 (s, 2H), 1.47 (q, J=10.8 Hz, 2H).

### Example 190:3-(5-(4-((4-(dimethylamino) piperidin-1-yl)methyl)-1-(oxetan-3-yl)-1H-pyrrolo[2,3-b]pyridin-6-yl)-1-oxoisoindolin-2-yl) piperidine-2,6-dione

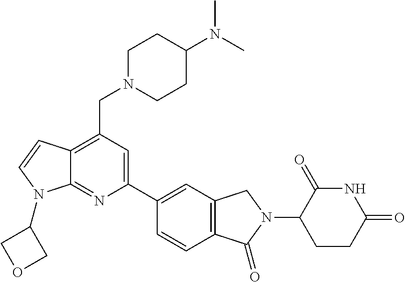 [SMILES: CN(C)C1CCN(Cc2cc(-c3ccc4c(c3)CN(C3CCC(=O)NC3=O)C4=O)nc3c2ccn3C2COC2)CC1]

The title compound was prepared in a manner analogous to Example 1 by reductive amination between N,N-dimethylpiperidin-4-amine and 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₃₁H₃₆N₆O₄, 556.28; m/z found, 557.39 [M+H]⁺. ¹H NMR (400 MHZ, DMSO-d₆) δ 11.02 (s, 1H), 8.38 (s, 1H), 8.33 (d, J=8.0 Hz, 1H), 8.24 (s, 1H), 7.98 (d, J=3.6 Hz, 1H), 7.84 (d, J=8.0 Hz, 1H), 7.77 (s, 1H), 6.80 (d, J=3.6 Hz, 1H), 6.15-6.09 (m, 1H), 5.18-5.13 (m, 1H), 5.08 (t, J=7.0 Hz, 4H), 4.57 (d, J=17.4 Hz, 1H), 4.44 (d, J=17.4 Hz, 1H), 3.84 (s, 2H), 2.99-2.88 (m, 3H), 2.63 (d, J=16.0 Hz, 1H), 2.44 (dd, J=13.2, 4.4 Hz, 1H), 2.41-2.35 (m, 1H), 2.32 (s, 6H), 2.05 (t, J=10.8 Hz, 3H), 1.79 (d, J=11.0 Hz, 2H), 1.50 (dd, J=20.8, 11.2 Hz, 2H).

### Example 191:3-(5-(4-((3-oxa-7-azabicyclo[3.3.1]nonan-7-yl)methyl)-1-(oxetan-3-yl)-1H-pyrrolo[2,3-b]pyridin-6-yl)-1-oxoisoindolin-2-yl) piperidine-2,6-dione

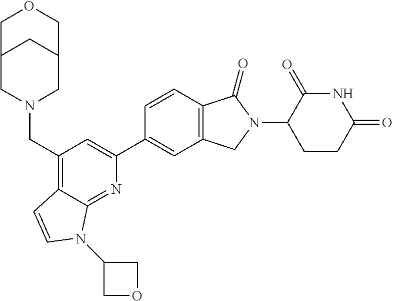 [SMILES: O=C1CCC(N2Cc3cc(-c4cc(CN5CC6COCC(C6)C5)c5ccn(C6COC6)c5n4)ccc3C2=O)C(=O)N1]

The title compound was prepared in a manner analogous to Example 1 by reductive amination between 3-oxa-7-azabicyclo[3.3.1]nonane and 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₃₁H₃₃N₅O₅, 555.25; m/z found, 556.3 [M+H]⁺. ¹H NMR (400 MHZ, DMSO-d₆) δ 11.00 (s, 1H), 8.37 (s, 1H), 8.32 (d, J=8.4 Hz, 1H), 8.18 (s, 1H), 7.95 (d, J=3.6 Hz, 1H), 7.88 (s, 1H), 7.83 (d, J=8.0 Hz, 1H), 7.07 (d, J=3.6 Hz, 1H), 6.18-6.08 (m, 1H), 5.18-5.13 (m, 1H), 5.07 (d, J=7.2 Hz, 4H), 4.57 (d, J=17.2 Hz, 1H), 4.43 (d, J=17.2 Hz, 1H), 3.84 (d, J=10.8 Hz, 2H), 3.80 (s, 2H), 3.69 (d, J=10.0 Hz, 2H), 2.99 (d, J=10.0 Hz, 2H), 2.92 (d, J=13.2 Hz, 1H), 2.62 (d, J=16.4 Hz, 1H), 2.46 (dd, J=7.4, 6.0 Hz, 1H), 2.39 (d, J=10.0 Hz, 2H), 2.09-2.00 (m, 1H), 1.79 (d, J=10.8 Hz, 1H), 1.71 (s, 2H), 1.61-1.55 (m, 1H).

### Example 192:1-((6-(2-(2,6-dioxopiperidin-3-yl)-1-oxoisoindolin-5-yl)-1-(oxetan-3-yl)-1H-pyrrolo[2,3-b]pyridin-4-yl)methyl) piperidine-4-carbonitrile

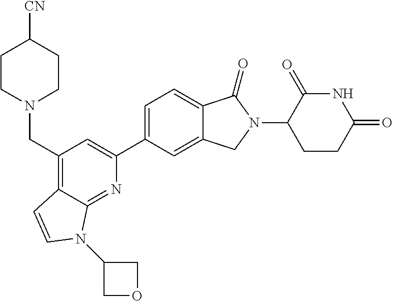 [SMILES: N#CC1CCN(Cc2cc(-c3ccc4c(c3)CN(C3CCC(=O)NC3=O)C4=O)nc3c2ccn3C2COC2)CC1]

The title compound was prepared in a manner analogous to Example 1 by reductive amination between piperidine-4-carbonitrile and 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₃₀H₃₀N₆O₄, 538.61; m/z found, 539.4 [M+H]⁺. ¹H NMR (400 MHZ, DMSO-d₆) δ 11.02 (s, 1H), 8.38 (s, 1H), 8.34 (d, J=8.0 Hz, 1H), 8.21 (s, 1H), 7.99 (d, J=3.6 Hz, 1H), 7.84 (d, J=8.0 Hz, 1H), 7.78 (s, 1H), 6.81 (d, J=3.6 Hz, 1H), 6.16-6.08 (m, 1H), 5.17-5.11 (m, 1H), 5.07 (d, J=7.2 Hz, 4H), 4.57 (d, J=17.4 Hz, 1H), 4.44 (d, J=17.4 Hz, 1H), 3.86 (s, 2H), 2.97-2.87 (m, 2H), 2.68-2.58 (m, 3H), 2.47-2.31 (m, 3H), 2.10-2.00 (m, 1H), 1.94-1.84 (m, 2H), 1.80-1.69 (m, 2H).

### Example 193:3-(5-(3-(isopropylamino)-7-(pyrrolidin-1-ylmethyl)-1H-pyrazolo[4,3-b]pyridin-5-yl)-1-oxoisoindolin-2-yl) piperidine-2,6-dione

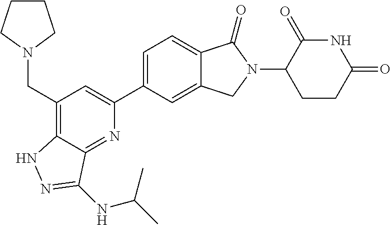 [SMILES: CC(C)Nc1n[nH]c2c(CN3CCCC3)cc(-c3ccc4c(c3)CN(C3CCC(=O)NC3=O)C4=O)nc12]

The title compound was prepared in a manner analogous to Example 173 by reductive amination between pyrrolidine and 5-chloro-3-(isopropylamino)-1-((2-(trimethylsilyl)ethoxy)methyl)-1H-pyrazolo[4,3-b]pyridine-7-carbaldehyde (Intermediate 25 or Intermediate 26) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of N-SEM group as a yellow solid. LC-MS (ESI): mass calced for: C₂₇H₃₁N₇O₃, 501.25; m/z found, 502.2 [M+H]⁺. ¹H NMR (400 MHZ, DMSO-d₆) δ 11.73 (s, 1H), 11.01 (s, 1H), 8.35 (s, 1H), 8.32 (d, J=8.0 Hz, 1H), 8.24 (s, 1H), 7.88 (s, 1H), 7.82 (d, J=8.0 Hz, 1H), 5.57 (d, J=8.0 Hz, 1H), 5.17-5.12 (m, 1H), 4.55 (d, J=17.2 Hz, 1H), 4.43 (d, J=17.2 Hz, 1H), 4.02-3.99 (m, 1H), 3.90 (s, 2H), 2.98-2.88 (m, 1H), 2.62 (d, J=17.6 Hz, 1H), 2.55 (s, 4H), 2.45 (dd, J=13.2, 4.2 Hz, 1H), 2.09-2.00 (m, 1H), 1.75 (s, 4H), 1.28 (d, J=6.4 Hz, 6H).

### Example 194:3-(5-(2-methyl-7-(pyrrolidin-1-ylmethyl) oxazolo[4,5-b]pyridin-5-yl)-1-oxoisoindolin-2-yl) piperidine-2,6-dione

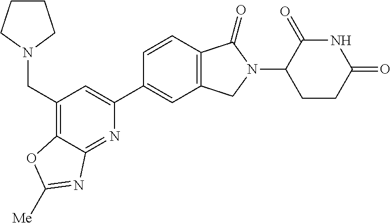 [SMILES: Cc1nc2nc(-c3ccc4c(c3)CN(C3CCC(=O)NC3=O)C4=O)cc(CN3CCCC3)c2o1]

The title compound was prepared in a manner analogous to Example 88 by displacement between pyrrolidine and 5-bromo-7-(bromomethyl)-2-methyloxazolo[4,5-b]pyridine (Intermediate 69a) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₅H₂₅N₅O₄, 459.61; m/z found, 460.5 [M+H]⁺. ¹H NMR (400 MHZ, DMSO-d₆) δ 11.09 (s, 1H), 8.40 (s, 1H), 8.31 (d, J=8.0 Hz, 1H), 8.26 (s, 1H), 8.11 (s, 1H), 7.92 (d, J=8.0 Hz, 1H), 5.24-5.19 (m, 1H), 4.64 (d, J=17.4 Hz, 1H), 4.51 (d, J=17.4 Hz, 1H), 4.07 (s, 2H), 3.06-2.94 (m, 1H), 2.79 (s, 3H), 2.73-2.64 (m, 5H), 2.54-2.45 (m, 1H), 2.14-2.10 (m, 1H), 1.82 (s, 4H).

### Example 195:3-(5-(7-methyl-2-(pyrrolidin-1-ylmethyl) oxazolo[4,5-b]pyridin-5-yl)-1-oxoisoindolin-2-yl) piperidine-2,6-dione

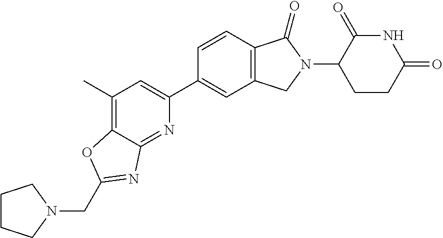 [SMILES: Cc1cc(-c2ccc3c(c2)CN(C2CCC(=O)NC2=O)C3=O)nc2nc(CN3CCCC3)oc12]

The title compound was prepared in a manner analogous to Example 88 by displacement between pyrrolidine and 5-bromo-2-(bromomethyl)-7-methyloxazolo[4,5-b]pyridine (Intermediate 69b) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₅H₂₅N₅O₄, 459.61; m/z found, 460.1 [M+H]⁺. ¹H NMR (400 MHZ, DMSO-d₆) δ 11.01 (s, 1H), 8.35 (s, 1H), 8.26 (d, J=8.0 Hz, 1H), 8.15 (s, 1H), 8.02 (s, 1H), 7.85 (d, J=8.0 Hz, 1H), 5.18-5.13 (m, 1H), 4.56 (d, J=17.4 Hz, 1H), 4.44 (d, J=17.4 Hz, 1H), 4.03 (s, 2H), 2.99-2.88 (m, 1H), 2.69-2.59 (m, 8H), 2.48-2.41 (m, 1H), 2.09-1.99 (m, 1H), 1.81-1.70 (m, 4H).

### Example 196:3-(5-(3-amino-7-((3-hydroxypyrrolidin-1-yl)methyl)-1H-pyrazolo[4,3-b]pyridin-5-yl)-1-oxoisoindolin-2-yl) piperidine-2,6-dione

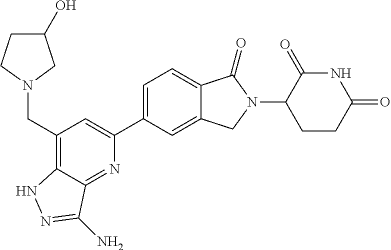 [SMILES: Nc1n[nH]c2c(CN3CCC(O)C3)cc(-c3ccc4c(c3)CN(C3CCC(=O)NC3=O)C4=O)nc12]

The title compound was prepared in a manner analogous to Example 173 by reductive amination between pyrrolidin-3-ol and 3-amino-5-chloro-1-((2-(trimethylsilyl)ethoxy)methyl)-1H-pyrazolo[4,3-b]pyridine-7-carbaldehyde (Intermediate 27) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of N-SEM group as a gray solid (TFA salt). LC-MS (ESI): mass calced for: C₂₄H₂₅N₇O₄, 475.20; m/z found, 476.3 [M+H]⁺. ¹H NMR (400 MHZ, DMSO-d₆) δ 11.02 (s, 1H), 8.37 (s, 1H), 8.33 (d, J=7.6 Hz, 1H), 8.22 (s, 1H), 7.89 (d, J=8.0 Hz, 1H), 5.54 (s, 1H), 5.18-5.14 (m, 1H), 4.68 (s, 2H), 4.58 (d, J=17.4 Hz, 1H), 4.45 (d, J=17.2 Hz, 2H), 3.59 (s, 4H), 2.98-2.89 (m, 1H), 2.64 (d, J=18.6 Hz, 1H), 2.46-2.42 (m, 1H), 2.09-1.96 (m, 3H). ¹⁹F NMR (376 MHZ, DMSO-d₆) δ -73.61 (ppm).

### Example 197:3-(5-(2-methyl-7-(pyrrolidin-1-ylmethyl) oxazolo [5,4-b]pyridin-5-yl)-1-oxoisoindolin-2-yl) piperidine-2,6-dione

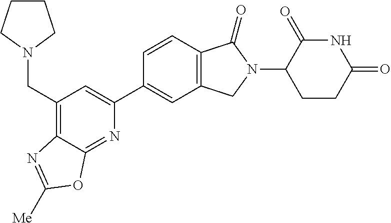 [SMILES: Cc1nc2c(CN3CCCC3)cc(-c3ccc4c(c3)CN(C3CCC(=O)NC3=O)C4=O)nc2o1]

The title compound was prepared in a manner analogous to Example 88 by displacement between pyrrolidine and 7-(bromomethyl)-5-chloro-2-methyloxazolo [5,4-b]pyridine (Intermediate 70a) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a light yellow solid. LC-MS (ESI): mass calcd. for C₂₅H₂₅N₅O₄, 459.2; m/z found, 460.2 [M+H]⁺. ¹H NMR (400 MHZ, DMSO-d₆) δ 11.08 (s, 1H), 8.39 (s, 1H), 8.30 (d, J=8.0 Hz, 1H), 8.12 (s, 1H), 7.92 (d, J=8.0 Hz, 1H), 5.24-5.19 (m, 1H), 4.63 (d, J=17.4 Hz, 1H), 4.50 (d, J=17.4 Hz, 1H), 4.07 (s, 2H), 3.06-2.93 (m, 1H), 2.75 (s, 3H), 2.71 (s, 1H), 2.63 (s, 4H), 2.50 (dd, J=13.0, 4.6 Hz, 1H), 2.15-2.07 (m, 1H), 1.80 (s, 4H).

### Example 198:3-(5-(7-methyl-2-(pyrrolidin-1-ylmethyl) oxazolo [5,4-b]pyridin-5-yl)-1-oxoisoindolin-2-yl) piperidine-2,6-dione

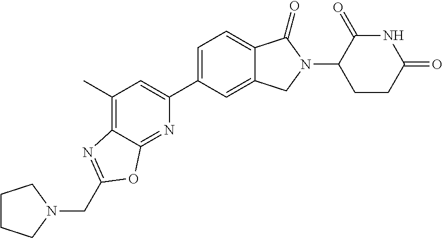 [SMILES: Cc1cc(-c2ccc3c(c2)CN(C2CCC(=O)NC2=O)C3=O)nc2oc(CN3CCCC3)nc12]

The title compound was prepared in a manner analogous to Example 88 by displacement between pyrrolidine and 2-(bromomethyl)-5-chloro-7-methyloxazolo [5,4-b]pyridine (Intermediate 70b) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a light yellow solid. LC-MS (ESI): mass calcd. for C₂₅H₂₅N₅O₄, 459.2; m/z found, 460.2 [M+H]⁺. ¹H NMR (400 MHZ, DMSO-d₆) δ 11.08 (s, 1H), 8.42 (s, 1H), 8.34 (d, J=8.0 Hz, 1H), 8.13 (s, 1H), 7.92 (d, J=8.0 Hz, 1H), 5.25-5.20 (m, 1H), 4.63 (d, J=17.2 Hz, 1H), 4.51 (d, J=17.2 Hz, 1H), 4.06 (s, 2H), 3.07-2.94 (m, 1H), 2.71-2.66 (m, 8H), 2.50-2.42 (m, 1H), 2.15-2.07 (m, 1H), 1.81 (s, 4H).

### Example 199:3-(5-(3-(ethylamino)-7-(pyrrolidin-1-ylmethyl)-1H-pyrazolo[4,3-b]pyridin-5-yl)-1-oxoisoindolin-2-yl) piperidine-2,6-dione

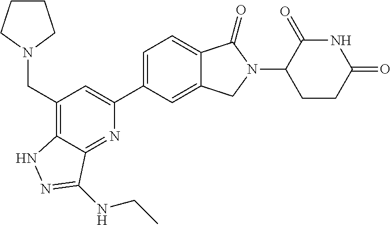 [SMILES: CCNc1n[nH]c2c(CN3CCCC3)cc(-c3ccc4c(c3)CN(C3CCC(=O)NC3=O)C4=O)nc12]

The title compound was prepared in a manner analogous to Example 173 by reductive amination between pyrrolidine and 5-chloro-3-(ethylamino)-1-((2-(trimethylsilyl)ethoxy)methyl)-1H-pyrazolo[4,3-b]pyridine-7-carbaldehyde (Intermediate 71) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of N-SEM group as a green oil (TFA salt). LC-MS (ESI): mass calced for: C₂₆H₂₉N₇O₃, 487.23; m/z found, 488.2 [M+H]⁺. ¹H NMR (400 MHZ, DMSO-d₆) δ 12.06 (s, 1H), 11.02 (s, 1H), 10.04 (s, 1H), 8.36 (s, 1H), 8.32 (d, J=8.2 Hz, 1H), 8.19 (s, 1H), 7.89 (d, J=8.0 Hz, 1H), 5.18-5.13 (m, 1H), 4.66 (s, 2H), 4.57 (d, J=17.4 Hz, 1H), 4.45 (d, J=17.4 Hz, 1H), 3.43-3.37 (m, 2H), 3.26 (s, 2H), 2.99-2.91 (m, 1H), 2.63 (d, J=18.4 Hz, 1H), 2.46-2.42 (m, 1H), 2.14-1.86 (m, 7H), 1.27 (d, J=7.2 Hz, 3H). ¹⁹F NMR (376 MHz, DMSO-d₆) δ-74.26 (ppm).

### Example 200:3-(1-oxo-5-(2-oxo-4-(pyrrolidin-1-ylmethyl)-2,3-dihydro-1H-pyrrolo[2,3-b]pyridin-6-yl) isoindolin-2-yl) piperidine-2,6-dione

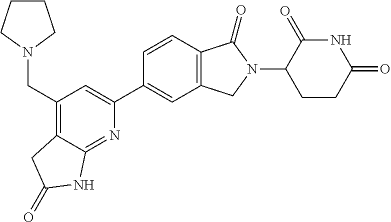 [SMILES: O=C1CCC(N2Cc3cc(-c4cc(CN5CCCC5)c5c(n4)NC(=O)C5)ccc3C2=O)C(=O)N1]

### Step A: 3-(1-oxo-5-(2-oxo-4-(pyrrolidin-1-ylmethyl)-1-((2-(trimethylsilyl)ethoxy)methyl)-2,3-dihydro-1H-pyrrolo[2,3-b]pyridin-6-yl) isoindolin-2-yl) piperidine-2,6-dione

To a solution of 6-chloro-4-(pyrrolidin-1-ylmethyl)-1-((2-(trimethylsilyl)ethoxy)methyl)-1,3-dihydro-2H-pyrrolo[2,3-b]pyridin-2-one (Intermediate 72, 15.0 mg, 39.3 μmol, 1.0 eq) in 1,4-Dioxane (2.00 mL) and water (0.20 mL) were added 3-(1-oxo-5-(4,4,5,5-tetramethyl-1,3,2-dioxaborolan-2-yl) isoindolin-2-yl) piperidine-2,6-dione (Intermediate 13, 17.4 mg, 47.1 μmol, 1.2 eq), Potassium phosphate tribasic (25.0 mg, 118 μmol, 3 eq), and 1,1′-Bis(di-t-butylphosphino) ferrocene palladium dichloride (2.56 mg, 3.93 μmol, 0.1 eq). The mixture was stirred under N₂ at 95° C. for 3 h. After cooled to room temperature, the reaction mixture was filtered and the filtrate was concentrated in vacuum. The crude product was purified by Prep-TLC(DCM/MeOH=10/1 v/v) to obtain 3-(1-oxo-5-(2-oxo-4-(pyrrolidin-1-ylmethyl)-1-((2-(trimethylsilyl)ethoxy)methyl)-2,3-dihydro-1H-pyrrolo[2,3-b]pyridin-6-yl) isoindolin-2-yl) piperidine-2,6-dione (10.0 mg, yield 43%) as a yellow solid. LC-MS (ESI): mass calced for: C₃₁H₃₉N₅O₅Si, 589.77; m/z found, 590.0 [M+H]⁺.

### Step B: 3-(1-oxo-5-(2-oxo-4-(pyrrolidin-1-ylmethyl)-2,3-dihydro-1H-pyrrolo[2,3-b]pyridin-6-yl) isoindolin-2-yl) piperidine-2,6-dione

To a solution of 3-(1-oxo-5-(2-oxo-4-(pyrrolidin-1-ylmethyl)-1-((2-(trimethylsilyl)ethoxy)methyl)-2,3-dihydro-1H-pyrrolo[2,3-b]pyridin-6-yl) isoindolin-2-yl) piperidine-2,6-dione (10.0 mg, 17.0 μmol, 1.0 eq) in DCM (2.00 mL) was added TFA (1.00 mL). The mixture was stirred at 25° C. for 16 h. After evaporation, the crude product was then purified by Prep-HPLC with YMC-Actus Triart C18 (5 μm, 21.2×250 mm), and mobile phase of 5-35% ACN in water (0.1% FA) over 15 min and 35-95% over 5 min then hold at 95% ACN for 3 min, at a flow rate of 20 mL/min to afford 3-(1-oxo-5-(2-oxo-4-(pyrrolidin-1-ylmethyl)-2,3-dihydro-1H-pyrrolo[2,3-b]pyridin-6-yl) isoindolin-2-yl) piperidine-2,6-dione formate (1.10 mg, yield 14%) as a yellow solid. LC-MS (ESI): mass calced for: C₂₅H₂₅N₅O₄, 459.51; m/z found, 460.5 [M+H]⁺. ¹H NMR (400 MHZ, DMSO-d₆) δ 11.12 (s, 1H), 11.02 (s, 1H), 8.22 (s, 1H), 8.15 (d, J=5.8 Hz, 2H), 7.83 (d, J=8.0 Hz, 1H), 7.59 (s, 1H), 7.40 (t, J=7.6 Hz, 1H), 7.25 (dd, J=31.2, 8.6 Hz, 1H), 6.54 (s, 1H), 5.15 (dd, J=13.2, 5.2 Hz, 1H), 4.55 (d, J=17.4 Hz, 1H), 4.42 (d, J=17.4 Hz, 1H), 3.63 (d, J=24.4 Hz, 2H), 2.97-2.89 (m, 1H), 2.65-2.54 (m, 5H), 2.43 (d, J=9.0 Hz, 1H), 2.04 (dd, J=11.2, 5.2 Hz, 1H), 1.75 (s, 4H).

### Example 201:3-(5-(3-amino-7-((3-fluoropyrrolidin-1-yl)methyl)-1H-pyrazolo[4,3-b]pyridin-5-yl)-1-oxoisoindolin-2-yl) piperidine-2,6-dione

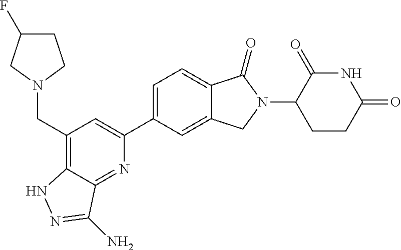 [SMILES: Nc1n[nH]c2c(CN3CCC(F)C3)cc(-c3ccc4c(c3)CN(C3CCC(=O)NC3=O)C4=O)nc12]

The title compound was prepared in a manner analogous to Example 173 by reductive amination between 3-fluoropyrrolidine HCl salt and 3-amino-5-chloro-1-((2-(trimethylsilyl)ethoxy)methyl)-1H-pyrazolo[4,3-b]pyridine-7-carbaldehyde (Intermediate 27) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of N-SEM group as a yellow solid. LC-MS (ESI): mass calced for: C₂₄H₂₄FN703, 477.19; m/z found, 478.2 [M+H]⁺. ¹H NMR (400 MHZ, DMSO-d₆) δ 11.77 (s, 1H), 11.01 (s, 1H), 8.35 (s, 1H), 8.31 (d, J=8.0 Hz, 1H), 8.15 (s, 1H), 7.89 (s, 1H), 7.83 (d, J=8.0 Hz, 1H), 5.46 (s, 2H), 5.30-5.12 (m, 2H), 4.55 (d, J=17.2 Hz, 1H), 4.43 (d, J=17.2 Hz, 1H), 3.95 (s, 2H), 3.02-2.71 (m, 5H), 2.62 (d, J=17.4 Hz, 1H), 2.44-2.40 (m, 1H), 2.26-2.09 (m, 1H), 2.08-1.99 (m, 1H), 1.99-1.84 (m, 1H). ¹⁹F NMR (400 MHZ, DMSO-d₆) δ-166.88 (ppm).

### Example 202:3-(5-(3-cyclopropyl-7-(pyrrolidin-1-ylmethyl)-1H-pyrazolo[4,3-b]pyridin-5-yl)-1-oxoisoindolin-2-yl) piperidine-2,6-dione

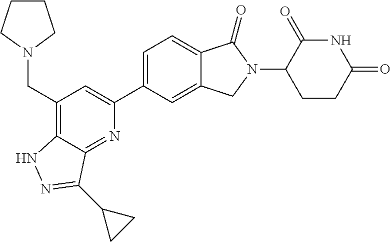 [SMILES: O=C1CCC(N2Cc3cc(-c4cc(CN5CCCC5)c5[nH]nc(C6CC6)c5n4)ccc3C2=O)C(=O)N1]

The title compound was prepared in a manner analogous to Example 173 by reductive amination between pyrrolidine and 5-chloro-3-cyclopropyl-1-((2-(trimethylsilyl)ethoxy)methyl)-1H-pyrazolo[4,3-b]pyridine-7-carbaldehyde (Intermediate 73) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of N-SEM group as a white solid. LC-MS (ESI): mass calced for C₂₇H₂₈N₆O₃:484.22; m/z found, 485.2 [M+H]⁺. ¹H NMR (400 MHZ, DMSO-d₆) δ 12.87 (s, 1H), 11.03 (s, 1H), 8.35 (s, 1H), 8.29 (d, J=8.0 Hz, 2H), 7.94 (s, 1H), 7.86 (d, J=8.0 Hz, 1H), 5.19-5.14 (m, 1H), 4.58 (d, J=17.4 Hz, 1H), 4.45 (d, J=17.4 Hz, 1H), 3.98 (s, 2H), 3.02-2.89 (m, 1H), 2.63 (d, J=17.4 Hz, 1H), 2.56 (s, 4H), 2.46-2.41 (m, 2H), 2.12-1.99 (m, 1H), 1.75 (s, 4H), 1.28-1.24 (m, 2H), 1.09-1.05 (m, 2H).

### Example 203:3-(5-(1-(oxetan-3-yl)-4-(pyrrolidin-1-ylmethyl)-1H-pyrazolo[3,4-b]pyridin-6-yl)-1-oxoisoindolin-2-yl) piperidine-2,6-dione

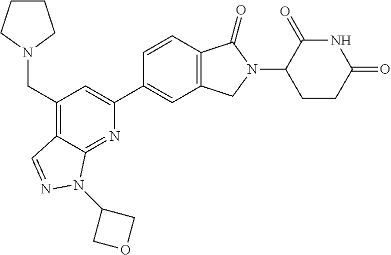 [SMILES: O=C1CCC(N2Cc3cc(-c4cc(CN5CCCC5)c5cnn(C6COC6)c5n4)ccc3C2=O)C(=O)N1]

### Step A: 6-chloro-1-(oxetan-3-yl)-4-(pyrrolidin-1-ylmethyl)-1H-pyrazolo[3,4-b]pyridine

To a solution of 6-chloro-4-(pyrrolidin-1-ylmethyl)-1H-pyrazolo[3,4-b]pyridine (Intermediate 36, 200 mg, 845 μmol, 1.0 eq) and 3-iodooxetane (233 mg, 1.27 mmol, 1.5 eq) in DMF (5.00 mL) was added potassium carbonate (350 mg, 2.53 mmol, 3.0 eq) and the mixture was stirred at 80° C. for 16 h. After cooled to room temperature, the mixture was poured into water (6 mL) and extracted with DCM (10 mL×3). The organic layer was dried over MgSO₄, filtered and concentrated under reduced pressure. The residue was purified by Prep-TLC(EA, 100% v/v) to give 6-chloro-1-(oxetan-3-yl)-4-(pyrrolidin-1-ylmethyl)-1H-pyrazolo[3,4-b]pyridine (70.0 mg, yield 25%) and 6-chloro-2-(oxetan-3-yl)-4-(pyrrolidin-1-ylmethyl)-2H-pyrazolo[3,4-b]pyridine (20.0 mg, yield 7%) as a yellow oil. LC-MS (ESI): mass calced for: C₁₄H₁₇ClN₄O, 292.1; m/z found, 293.1 [M+H]⁺. ¹H NMR (400 MHZ, DMSO-d₆) δ 8.45 (s, 1H), 7.25 (s, 1H), 6.08-6.04 (m, 1H), 5.04-4.98 (m, 4H), 3.96 (s, 2H), 2.51 (s, 2H), 2.50 (s, 2H), 1.73 (t, J=3.4 Hz, 4H).

### Step B: 3-(5-(1-(oxetan-3-yl)-4-(pyrrolidin-1-ylmethyl)-1H-pyrazolo[3,4-b]pyridin-6-yl)-1-oxoisoindolin-2-yl) piperidine-2,6-dione

To a solution of 6-chloro-1-(oxetan-3-yl)-4-(pyrrolidin-1-ylmethyl)-1H-pyrazolo[3,4-b]pyridine (70.0 mg, 239 μmol, 1.0 eq) and 3-(1-oxo-5-(4,4,5,5-tetramethyl-1,3,2-dioxaborolan-2-yl) isoindolin-2-yl) piperidine-2,6-dione (Intermediate 13, 106 mg, 287 μmol, 1.2 eq) in dioxane (2 mL) and water (0.2 mL) were added PdCl₂ (dtbpf) (15.6 mg, 23.9 μmol, 0.1 eq) and potassium phosphate (152 mg, 717 μmol, 3.0 eq). The mixture was stirred under N₂ at 95° C. for 1 h. The reaction mixture was cooled to room temperature and concentrated under reduced pressure. The residue was poured into water (5 mL) and extracted with EtOAc (10 mL×3). The organic layer was dried over MgSO₄, filtered and concentrated under reduced pressure. The residue was purified by Prep-TLC(DCM/MeOH=8/1 v/v) to obtain 3-(5-(1-(oxetan-3-yl)-4-(pyrrolidin-1-ylmethyl)-1H-pyrazolo[3,4-b]pyridin-6-yl)-1-oxoisoindolin-2-yl) piperidine-2,6-dione (27.9 mg, yield 22%) as a brown solid. LC-MS (ESI): mass calced for: C₂₇H₂₈N₆O₄, 500.1; m/z found, 501.1 [M+H]⁺. ¹H NMR (400 MHZ, DMSO-d₆) δ 11.02 (s, 1H), 8.46 (s, 2H), 8.39 (d, J=7.8 Hz, 1H), 7.89 (d, J=8.0 Hz, 2H), 6.33-6.28 (m, 1H), 5.21-5.16 (m, 1H), 5.13-5.08 (m, 4H), 4.59 (d, J=17.4 Hz, 1H), 4.46 (d, J=17.4 Hz, 1H), 4.06 (s, 2H), 2.92 (dd, J=12.9, 4.6 Hz, 1H), 2.63 (d, J=17.8 Hz, 3H), 2.47-2.39 (m, 3H), 2.06-2.03 (m, 1H), 1.78 (s, 4H).

### Example 204:3-(5-(3-(oxetan-3-ylamino)-7-(pyrrolidin-1-ylmethyl)-1H-pyrazolo[4,3-b]pyridin-5-yl)-1-oxoisoindolin-2-yl) piperidine-2,6-dione

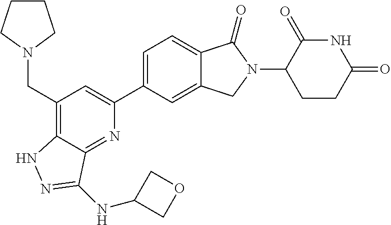 [SMILES: O=C1CCC(N2Cc3cc(-c4cc(CN5CCCC5)c5[nH]nc(NC6COC6)c5n4)ccc3C2=O)C(=O)N1]

The title compound was prepared in a manner analogous to Example 173 by reductive amination between pyrrolidine and 5-chloro-3-(oxetan-3-ylamino)-1-((2-(trimethylsilyl)ethoxy)methyl)-1H-pyrazolo[4,3-b]pyridine-7-carbaldehyde (Intermediate 74) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of N-SEM group as a yellow solid. LC-MS (ESI): mass calced for: C₂₇H₂₉N₇O₄, 515.23; m/z found, 516.2 [M+H]⁺. ¹H NMR (400 MHZ, DMSO-d₆) δ 13.30 (s, 1H), 11.06 (s, 1H), 8.14 (s, 1H), 8.06 (d, J=8.4 Hz, 2H), 7.93 (d, J=7.8 Hz, 1H), 7.34 (s, 1H), 5.25-5.16 (m, 1H), 4.73 (d, J=18.6 Hz, 3H), 4.62 (d, J=18.8 Hz, 1H), 4.54-4.47 (m, 1H), 3.68-3.54 (m, 6H), 3.19-3.07 (m, 2H), 2.94 (d, J=12.2 Hz, 1H), 2.64 (d, J=16.6 Hz, 1H), 2.44 (d, J=17.0 Hz, 1H), 1.97-1.84 (m, 5H).

### Example 205:3-(5-(3-amino-7-((4-hydroxypiperidin-1-yl)methyl)-1H-pyrazolo[4,3-b]pyridin-5-yl)-1-oxoisoindolin-2-yl) piperidine-2,6-dione

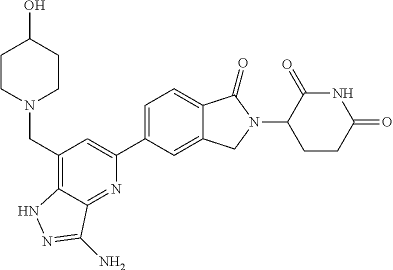 [SMILES: Nc1n[nH]c2c(CN3CCC(O)CC3)cc(-c3ccc4c(c3)CN(C3CCC(=O)NC3=O)C4=O)nc12]

The title compound was prepared in a manner analogous to Example 173 by reductive amination between piperidin-4-ol and 3-amino-5-chloro-1-((2-(trimethylsilyl)ethoxy)methyl)-1H-pyrazolo[4,3-b]pyridine-7-carbaldehyde (Intermediate 27) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of N-SEM group as a gray solid. LC-MS (ESI): mass calced for: C₂₅H₂₇N₇O, 489.21; m/z found, 490.3 [M+H]⁺. ¹H NMR (400 MHZ, DMSO-d₆) δ 11.69 (s, 1H), 11.01 (s, 1H), 8.35 (s, 1H), 8.31 (d, J=8.0 Hz, 1H), 8.15 (s, 1H), 7.86 (s, 1H), 7.84 (d, J=8.0 Hz, 1H), 5.46 (s, 2H), 5.33 (t, J=4.6 Hz, 1H), 5.18-5.13 (m, 1H), 4.56 (d, J=17.2 Hz, 1H), 4.43 (d, J=17.2 Hz, 1H), 3.77 (s, 2H), 2.99-2.89 (m, 1H), 2.78-2.73 (m, 2H), 2.63 (d, J=15.8 Hz, 1H), 2.47-2.43 (m, 1H), 2.16 (t, J=9.2 Hz, 2H), 2.04 (d, J=6.4 Hz, 1H), 1.73 (d, J=9.6 Hz, 2H), 1.49-1.44 (m, 2H).

### Example 206:3-(5-(1-cyclobutyl-4-(pyrrolidin-1-ylmethyl)-1H-pyrazolo[3,4-b]pyridin-6-yl)-1-oxoisoindolin-2-yl) piperidine-2,6-dione

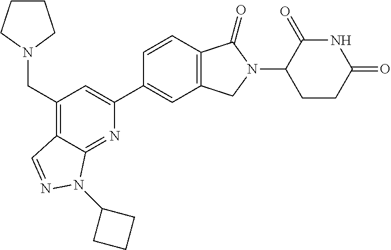 [SMILES: O=C1CCC(N2Cc3cc(-c4cc(CN5CCCC5)c5cnn(C6CCC6)c5n4)ccc3C2=O)C(=O)N1]

The title compound was prepared in a manner analogous to Example 203 by alkylation of 6-chloro-4-(pyrrolidin-1-ylmethyl)-1H-pyrazolo[3,4-b]pyridine (Intermediate 36) with bromocyclobutane followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid (TFA salt). LC-MS (ESI): mass calced for: C₂₈H₃₀N₆O₃, 498.2; m/z found, 499.1 [M+H]⁺. ¹H NMR (400 MHZ, DMSO-d₆) δ 11.03 (s, 1H), 10.29 (s, 1H), 8.54 (s, 1H), 8.45 (s, 1H), 8.38 (d, J=8.0 Hz, 1H), 8.14 (s, 1H), 7.95 (d, J=8.0 Hz, 1H), 5.71-5.55 (m, 1H), 5.20-5.15 (m, 1H), 4.83 (s, 2H), 4.61 (d, J=17.4 Hz, 1H), 4.48 (d, J=17.4 Hz, 1H), 3.55 (s, 2H), 3.24 (s, 4H), 2.93 (dd, J=22.0, 8.8 Hz, 1H), 2.79-2.69 (m, 2H), 2.64 (d, J=17.8 Hz, 1H), 2.45 (s, 1H), 2.06 (d, J=12.8 Hz, 3H), 1.98-1.83 (m, 4H). ¹⁹F NMR (400 MHZ, DMSO-d₆) δ-73.57 (ppm).

### Example 207:3-(5-(3-amino-7-((3-fluoroazetidin-1-yl)methyl)-1H-pyrazolo[4,3-b]pyridin-5-yl)-1-oxoisoindolin-2-yl) piperidine-2,6-dione

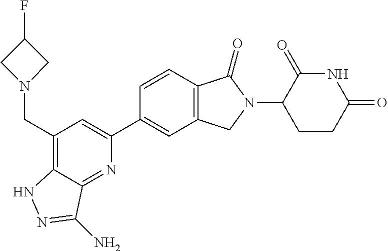 [SMILES: Nc1n[nH]c2c(CN3CC(F)C3)cc(-c3ccc4c(c3)CN(C3CCC(=O)NC3=O)C4=O)nc12]

The title compound was prepared in a manner analogous to Example 173 by reductive amination between 3-fluoroazetidine 3-amino-5-chloro-1-((2-(trimethylsilyl)ethoxy)methyl)-1H-pyrazolo[4,3-b]pyridine-7-carbaldehyde (Intermediate 27) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of N-SEM group

as a yellow solid. LC-MS (ESI): mass calced for: C₂₃H₂₂FN703, 463.18; m/z found, 464.1 [M+H]⁺. ¹H NMR (400 MHZ, DMSO-d₆) δ 11.02 (s, 1H), 8.37 (s, 1H), 8.32 (d, J=8.0 Hz, 1H), 8.11 (s, 1H), 7.88 (d, J=8.0 Hz, 1H), 5.55-5.38 (m, 1H), 5.19-5.13 (m, 1H), 4.73 (s, 2H), 4.59-4.42 (m, 8H), 2.98-2.89 (m, 1H), 2.63 (d, J=16.6 Hz, 1H), 2.48-2.40 (m, 1H), 2.10-2.00 (m, 1H). ¹⁹F NMR (400 MHZ, DMSO-d₆) δ-74.07 (ppm).

### Example 208:3-(5-(3-amino-7-(azetidin-1-ylmethyl)-1H-pyrazolo[4,3-b]pyridin-5-yl)-1-oxoisoindolin-2-yl) piperidine-2,6-dione

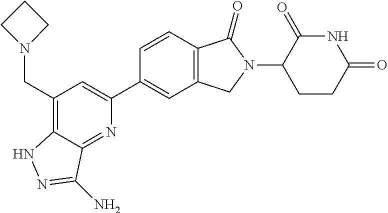 [SMILES: Nc1n[nH]c2c(CN3CCC3)cc(-c3ccc4c(c3)CN(C3CCC(=O)NC3=O)C4=O)nc12]

The title compound was prepared in a manner analogous to Example 173 by reductive amination between azetidine and 3-amino-5-chloro-1-((2-(trimethylsilyl)ethoxy)methyl)-1H-pyrazolo[4,3-b]pyridine-7-carbaldehyde (Intermediate 27) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of N-SEM group as a yellow solid. LC-MS (ESI): mass calced for: C₂₃H₂₃N₇O₃, 445.19; m/z found, 446.2 [M+H]⁺. ¹H NMR (400 MHZ, DMSO-d₆) δ 11.89 (s, 1H), 11.02 (s, 1H), 8.35 (s, 1H), 8.30 (d, J=8.0 Hz, 1H), 8.13 (s, 1H), 7.87 (t, J=15.2 Hz, 2H), 5.52 (s, 2H), 5.18-5.13 (m, 1H), 4.56 (d, J=17.2 Hz, 1H), 4.44 (d, J=17.2 Hz, 1H), 4.21-3.94 (m, 2H), 3.58-3.46 (m, 4H), 2.96-2.89 (m, 1H), 2.63 (d, J=15.8 Hz, 1H), 2.46-2.42 (m, 1H), 2.24-2.11 (m, 2H), 2.05-2.02 (m, 1H).

### Example 209:3-(5-(3-cyclobutyl-7-(pyrrolidin-1-ylmethyl)-3H-imidazo[4,5-b]pyridin-5-yl)-1-oxoisoindolin-2-yl) piperidine-2,6-dione

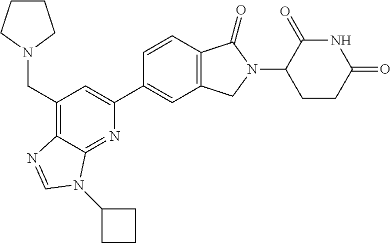 [SMILES: O=C1CCC(N2Cc3cc(-c4cc(CN5CCCC5)c5ncn(C6CCC6)c5n4)ccc3C2=O)C(=O)N1]

The title compound was prepared in a manner analogous to Example 88 by displacement between pyrrolidine and 7-(bromomethyl)-5-chloro-3-cyclobutyl-3H-imidazo[4,5-b]pyridine (Intermediate 75) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₈H₃₀N₆O₃, 498.59; m/z found, 499.2 [M+H]⁺. ¹H NMR (400 MHZ, DMSO-d₆) δ 11.02 (s, 1H), 8.94 (s, 1H), 8.38 (s, 1H), 8.32 (d, J=7.8 Hz, 1H), 8.07 (s, 2H), 7.90 (d, J=8.0 Hz, 1H), 5.34-5.23 (m, 1H), 5.18-5.13 (m, 1H), 4.59 (d, J=17.4 Hz, 1H), 4.46 (d, J=17.4 Hz, 1H), 4.35 (s, 2H), 2.93 (dd, J=21.8, 9.0 Hz, 3H), 2.82-2.74 (m, 2H), 2.60 (dd, J=26.6, 11.8 Hz, 3H), 2.43 (d, J=4.3 Hz, 1H), 2.09-2.02 (m, 1H), 2.00-1.93 (m, 2H), 1.88 (s, 4H), 1.55-1.42 (m, 1H), 1.30-1.26 (m, 1H).

### Example 210:3-(5-(3-amino-1-cyclopropyl-7-(pyrrolidin-1-ylmethyl)-1H-pyrazolo[4,3-b]pyridin-5-yl)-1-oxoisoindolin-2-yl) piperidine-2,6-dione

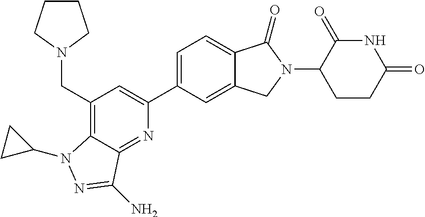 [SMILES: Nc1nn(C2CC2)c2c(CN3CCCC3)cc(-c3ccc4c(c3)CN(C3CCC(=O)NC3=O)C4=O)nc12]

The title compound was prepared in a manner analogous to Example 1 Suzuki coupling of 5-chloro-1-cyclopropyl-7-(pyrrolidin-1-ylmethyl)-1H-pyrazolo[4,3-b]pyridin-3-amine (Intermediate 76) with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₇H₂₉N₇O₃, 499.2; m/z found, 500.2 [M+H]⁺. ¹H NMR (400 MHZ, DMSO-d₆) δ 11.02 (s, 1H), 8.36 (s, 1H), 8.32 (d, J=8.0 Hz, 1H), 8.20 (s, 1H), 7.94 (s, 1H), 7.84 (d, J=8.0 Hz, 1H), 5.53 (s, 2H), 5.18-5.14 (m, 1H), 4.56 (d, J=17.2 Hz, 1H), 4.43 (d, J=17.2 Hz, 1H), 4.17 (s, 2H), 3.94-3.89 (m, 1H), 3.00-2.86 (m, 1H), 2.65-2.60 (m, 5H), 2.47-2.36 (m, 1H), 2.11-2.02 (m, 1H), 1.74 (s, 4H), 1.21-1.13 (m, 2H), 1.07-0.95 (m, 2H).

### Example 211:3-(5-(3-(oxetan-3-yl)-7-(pyrrolidin-1-ylmethyl)-3H-imidazo[4,5-b]pyridin-5-yl)-1-oxoisoindolin-2-yl) piperidine-2,6-dione

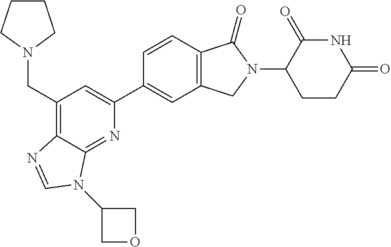 [SMILES: O=C1CCC(N2Cc3cc(-c4cc(CN5CCCC5)c5ncn(C6COC6)c5n4)ccc3C2=O)C(=O)N1]

The title compound was prepared in a manner analogous to Example 88 by displacement between pyrrolidine and 7-(bromomethyl)-5-chloro-3-(oxetan-3-yl)-3H-imidazo[4,5-b]pyridine (Intermediate 77) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₇H₂₈N₆O₄, 500.22; m/z found, 501.3 [M+H]⁺. ¹H NMR (400 MHZ, DMSO-d₆) δ 11.02 (s, 1H), 8.76 (s, 1H), 8.36 (s, 1H), 8.31 (d, J=9.0 Hz, 1H), 8.17 (s, 1H), 7.98 (s, 1H), 7.86 (d, J=8.0 Hz, 1H), 5.98-5.91 (m, 1H), 5.24 (t, J=6.8 Hz, 2H), 5.18-5.13 (m, 1H), 5.06 (t, J=7.4 Hz, 2H), 4.58 (d, J=17.4 Hz, 1H), 4.45 (d, J=17.4 Hz, 1H), 4.10 (s, 2H), 2.95-2.89 (m, 1H), 2.65-2.58 (m, 5H), 2.46-2.38 (m, 1H), 2.07-2.03 (m, 1H), 1.74 (s, 4H).

### Example 212:3-(5-(3-amino-7-((4-fluoropiperidin-1-yl)methyl)-1H-pyrazolo[4,3-b]pyridin-5-yl)-1-oxoisoindolin-2-yl) piperidine-2,6-dione

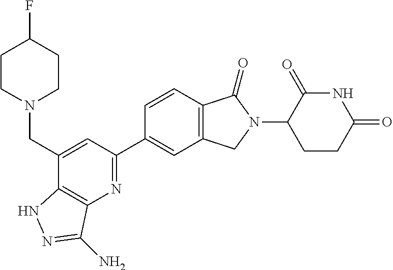 [SMILES: Nc1n[nH]c2c(CN3CCC(F)CC3)cc(-c3ccc4c(c3)CN(C3CCC(=O)NC3=O)C4=O)nc12]

The title compound was prepared in a manner analogous to Example 173 by reductive amination between 4-fluoropiperidine and 3-amino-5-chloro-1-((2-(trimethylsilyl)ethoxy)methyl)-1H-pyrazolo[4,3-b]pyridine-7-carbaldehyde (Intermediate 27) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of N-SEM group as a yellow solid. LC-MS (ESI): mass calced for: C₂₅H₂₆FN703, 491.21; m/z found, 492.2 [M+H]⁺. ¹H NMR (400 MHZ, DMSO-d₆) δ 11.02 (s, 1H), 10.07 (s, 1H), 8.36 (s, 1H), 8.32 (d, J=8.0 Hz, 1H), 8.15 (s, 1H), 7.89 (d, J=8.0 Hz, 1H), 5.18-5.13 (m, 1H), 4.99 (d, J=45.4 Hz, 1H), 4.61-4.53 (m, 3H), 4.45 (d, J=17.4 Hz, 1H), 4.04 (s, 2H), 3.49-3.22 (m, 4H), 2.99-2.89 (m, 1H), 2.63 (d, J=17.2 Hz, 1H), 2.44 (dd, J=13.0, 8.6 Hz, 1H), 2.15-1.94 (m, 5H). ¹⁹F NMR (400 MHZ, DMSO-d₆) δ-74.04,-187.41 (ppm).

### Example 213:3-(5-(3-amino-7-((3-hydroxyazetidin-1-yl)methyl)-1H-pyrazolo[4,3-b]pyridin-5-yl)-1-oxoisoindolin-2-yl) piperidine-2,6-dione

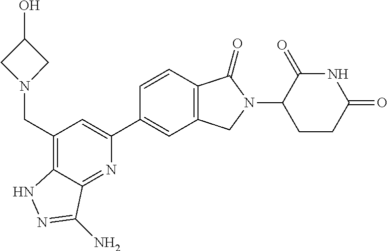 [SMILES: Nc1n[nH]c2c(CN3CC(O)C3)cc(-c3ccc4c(c3)CN(C3CCC(=O)NC3=O)C4=O)nc12]

The title compound was prepared in a manner analogous to Example 173 by reductive amination between azetidin-3-ol hydrochloride and 3-amino-5-chloro-1-((2-(trimethylsilyl)ethoxy)methyl)-1H-pyrazolo[4,3-b]pyridine-7-carbaldehyde (Intermediate 27) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of N-SEM group as a yellow solid. LC-MS (ESI): mass calced for: C₂₃H₂₃N₇O₄, 461.18; m/z found, 462.2 [M+H]⁺. ¹H NMR (400 MHz, DMSO-d₆) δ 11.02 (s, 1H), 8.37 (s, 1H), 8.32 (d, J=8.0 Hz, 1H), 8.12 (s, 1H), 7.89 (d, J=8.2 Hz, 1H), 5.19-5.14 (m, 1H), 4.69 (s, 2H), 4.55 (s, 1H), 4.45 (d, J=17.2 Hz, 2H), 4.39-4.33 (m, 2H), 4.08 (s, 2H), 2.93 (d, J=12.2 Hz, 1H), 2.61 (s, 1H), 2.43 (d, J=13.2 Hz, 1H), 2.09-2.02 (m, 1H).

### Example 214:3-(5-(3-amino-7-((tetrahydro-1H-furo[3,4-c]pyrrol-5 (3H)-yl)methyl)-1H-pyrazolo[4,3-b]pyridin-5-yl)-1-oxoisoindolin-2-yl) piperidine-2,6-dione

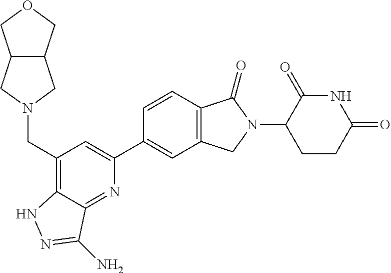 [SMILES: Nc1n[nH]c2c(CN3CC4COCC4C3)cc(-c3ccc4c(c3)CN(C3CCC(=O)NC3=O)C4=O)nc12]

The title compound was prepared in a manner analogous to Example 173 by reductive amination between hexahydro-1H-furo[3,4-c]pyrrole hydrochloride and 3-amino-5-chloro-1-((2-(trimethylsilyl)ethoxy)methyl)-1H-pyrazolo[4,3-b]pyridine-7-carbaldehyde (Intermediate 27) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of N-SEM group as a yellow solid. LC-MS (ESI): mass calced for: C₂₆H₂₇N₇O₄, 501.21; m/z found, 502.2 [M+H]⁺. ¹H NMR (400 MHZ, DMSO-d₆) δ 11.02 (s, 1H), 8.36 (s, 1H), 8.31 (d, J=7.4 Hz, 1H), 8.16 (s, 1H), 7.90 (d, J=8.0 Hz, 1H), 5.17-5.13 (m, 1H), 4.57 (d, J=17.6 Hz, 3H), 4.47 (s, 1H), 3.85-3.76 (m, 4H), 2.95 (d, J=4.8 Hz, 5H), 2.60 (d, J=8.2 Hz, 1H), 2.39 (d, J=13.0 Hz, 1H), 2.10-1.96 (m, 3H).

### Example 215:3-(5-(7-((3-oxa-7-azabicyclo[3.3.1]nonan-7-yl)methyl)-3-amino-1H-pyrazolo[4,3-b]pyridin-5-yl)-1-oxoisoindolin-2-yl) piperidine-2,6-dione

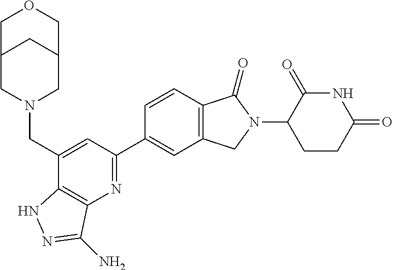 [SMILES: Nc1n[nH]c2c(CN3CC4COCC(C4)C3)cc(-c3ccc4c(c3)CN(C3CCC(=O)NC3=O)C4=O)nc12]

The title compound was prepared in a manner analogous to Example 173 by reductive amination between 3-Oxa-7-azabicyclo[3.3.1]nonane hydrochloride and 3-amino-5-chloro-1-((2-(trimethylsilyl)ethoxy)methyl)-1H-pyrazolo[4,3-b]pyridine-7-carbaldehyde (Intermediate 27) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of N-SEM group

as a yellow solid (TFA salt). LC-MS (ESI): mass calced for: C₂₇H₂₉N₇O₄, 515.23; m/z found, 516.2 [M+H]⁺. ¹H NMR (400 MHZ, DMSO-d₆) δ 11.02 (s, 1H), 9.08 (s, 1H), 8.35 (s, 1H), 8.30 (d, J=8.0 Hz, 1H), 8.25 (s, 1H), 7.91 (d, J=8.0 Hz, 1H), 5.18-5.13 (m, 1H), 4.61-4.53 (m, 3H), 4.45 (d, J=17.4 Hz, 1H), 4.00 (d, J=10.8 Hz, 2H), 3.73-3.60 (m, 6H), 2.99-2.86 (m, 1H), 2.63 (d, J=16.8 Hz, 1H), 2.45-2.39 (m, 1H), 2.04 (s, 3H), 1.93 (d, J=13.4 Hz, 1H), 1.82 (d, J=13.4 Hz, 1H). ¹⁹F NMR (400 MHZ, DMSO-d₆) δ-74.95 (ppm).

### Example 216:3-(5-(7-((2-oxa-7-azaspiro[3.5]nonan-7-yl)methyl)-3-amino-1H-pyrazolo[4,3-b]pyridin-5-yl)-1-oxoisoindolin-2-yl) piperidine-2,6-dione

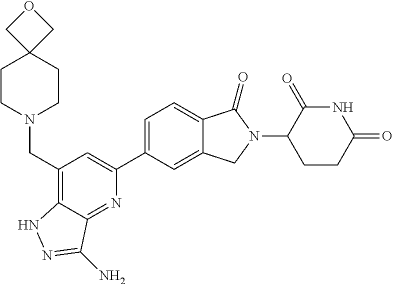 [SMILES: Nc1n[nH]c2c(CN3CCC4(CC3)COC4)cc(-c3ccc4c(c3)CN(C3CCC(=O)NC3=O)C4=O)nc12]

The title compound was prepared in a manner analogous to Example 173 by reductive amination between 2-oxa-7-azaspiro[3.5]nonane and 3-amino-5-chloro-1-((2-(trimethylsilyl) ethoxy)methyl)-1H-pyrazolo[4,3-b]pyridine-7-carbaldehyde (Intermediate 27) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of N-SEM group as a gray solid. LC-MS (ESI): mass calced for: C₂₇H₂₉N₇O₄, 515.23; m/z found, 516.4 [M+H]⁺. ¹H NMR (400 MHz, DMSO-d₆) δ 12.05 (s, 1H), 11.03 (s, 1H), 9.75 (s, 1H), 8.36 (s, 1H), 8.31 (d, J=8.0 Hz, 1H), 8.09 (s, 1H), 7.90 (d, J=8.0 Hz, 1H), 5.18-5.13 (m, 1H), 4.60-4.31 (m, 8H), 3.58 (s, 2H), 2.99-2.91 (m, 3H), 2.64 (d, J=17.6 Hz, 1H), 2.45-2.42 (m, 1H), 2.25 (s, 2H), 2.09-2.00 (m, 3H), 1.83 (s, 2H). ¹⁹F NMR (376 MHZ, DMSO-d₆) δ-73.71 (ppm).

### Example 217:3-(5-(7-cyclobutyl-4-(pyrrolidin-1-ylmethyl)-7H-pyrrolo[2,3-d]pyrimidin-2-yl)-1-oxoisoindolin-2-yl) piperidine-2,6-dione

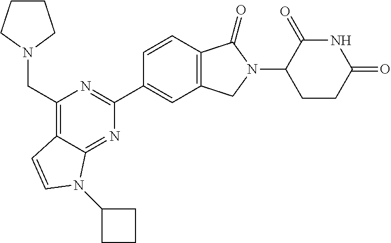 [SMILES: O=C1CCC(N2Cc3cc(-c4nc(CN5CCCC5)c5ccn(C6CCC6)c5n4)ccc3C2=O)C(=O)N1]

The title compound was prepared in a manner analogous to Example 203 by alkylation of chloro-4-(pyrrolidin-1-ylmethyl)-7H-pyrrolo[2,3-d]pyrimidine (Intermediate 78) with bromocyclobutane followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as an off-white solid. LC-MS (ESI): mass calced for C₂₈H₃₀N₆O₃:498; m/z found, 499.2 [M+H]⁺. ¹H NMR (400 MHz, DMSO-d₆) δ 11.02 (s, 1H), 8.67 (d, J=10.4 Hz, 2H), 8.16 (s, 1H), 7.91 (d, J=3.6 Hz, 1H), 7.87 (d, J=8.0 Hz, 1H), 6.86 (d, J=3.6 Hz, 1H), 5.46-5.40 (m, 1H), 5.18-5.13 (m, 1H), 4.60 (d, J=17.4 Hz, 1H), 4.47 (d, J=17.4 Hz, 1H), 4.09 (s, 2H), 2.99-2.88 (m, 1H), 2.63-2.52 (m, 8H), 2.46-2.42 (m, 1H), 2.11-2.00 (m, 1H), 1.97-1.85 (m, 2H), 1.74 (s, 4H).

### Example 218:3-(5-(7-(oxetan-3-yl)-4-(pyrrolidin-1-ylmethyl)-7H-pyrrolo[2,3-d]pyrimidin-2-yl)-1-oxoisoindolin-2-yl) piperidine-2,6-dione

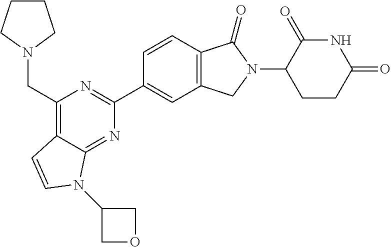 [SMILES: O=C1CCC(N2Cc3cc(-c4nc(CN5CCCC5)c5ccn(C6COC6)c5n4)ccc3C2=O)C(=O)N1]

The title compound was prepared in a manner analogous to Example 203 by alkylation of chloro-4-(pyrrolidin-1-ylmethyl)-7H-pyrrolo[2,3-d]pyrimidine (Intermediate 78) with oxetan-3-yl methanesulfonate followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₇H₂₈N₆O₄, 500.22; m/z found, 501.2 [M+H]⁺. ¹H NMR (400 MHz, DMSO-d₆) δ 11.08 (s, 1H), 8.75 (s, 1H), 8.73 (d, J=8.0 Hz, 1H), 8.24 (s, 1H), 8.11 (d, J=3.6 Hz, 1H), 7.93 (d, J=8.0 Hz, 1H), 7.01 (d, J=3.6 Hz, 1H), 6.26-6.11 (m, 1H), 5.23-5.21 (m, 1H), 5.17-5.08 (m, 4H), 4.66 (d, J=17.4 Hz, 1H), 4.52 (d, J=17.4 Hz, 1H), 4.16 (s, 2H), 3.05-2.97 (m, 1H), 2.70-2.62 (m, 5H), 2.53-2.41 (m, 1H), 2.13-2.10 (m, 1H), 1.81 (s, 4H).

### Example 219: (S)-3-(5-(1-methyl-4-(((R)-pyrrolidin-3-yl)oxy)-1H-pyrrolo[2,3-b]pyridin-6-yl)-1-oxoisoindolin-2-yl) piperidine-2,6-dione

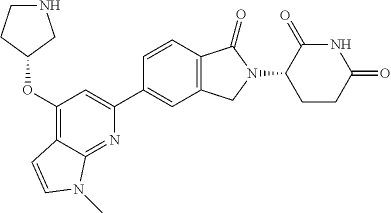 [SMILES: Cn1ccc2c(O[C@@H]3CCNC3)cc(-c3ccc4c(c3)CN([C@H]3CCC(=O)NC3=O)C4=O)nc21]

### Step A: 4,6-dichloro-1-methyl-1H-pyrrolo[2,3-b]pyridine

To a stirred mixture of 4,6-dichloro-1H-pyrrolo[2,3-b]pyridine (1.00 g, 5.35 mmol, 1.0 eq) in DMF (15.0 mL) were added K₂CO₃ (2.22 g, 16.0 mmol, 3.0 eq) and MeI (1.52 g, 669 μL, 10.7 mmol, 2.0 eq). The resulting mixture was stirred at 25° C. for 2 h. The reaction mixture was diluted with H₂O (50 mL) and extracted with EtOAc (50 mL×3). The combined organic layers were washed with brine (50 mL×4), dried over anhydrous Na₂SO₄, filtered and concentrated under reduced pressure. The residue was purified by flash column chromatography on silica gel (PE/EtOAc=3/1 v/v) to give 4,6-dichloro-1-methyl-1H-pyrrolo[2,3-b]pyridine (1.00 g, yield 93%) as a white solid. LC-MS: 201 (M+H)+. Revised as the following: LC-MS (ESI): mass calcd. for C₈H₆C₁₂N₂, 199.99; m/z found, 201.2 [M+H]⁺.

### Step B: tert-butyl (R)-3-((6-chloro-1-methyl-1H-pyrrolo[2,3-b]pyridin-4-yl)oxy) pyrrolidine-1-carboxylate

To a solution of tert-butyl (R)-3-hydroxypyrrolidine-1-carboxylate (300 mg, 1.60 mmol, 1.0 eq) in DMF (10.0 mL) was added Sodium hydride (60% suspend in oil) (57.6 mg, 2.40 mmol, 1.5 eq). The reaction mixture was stirred at 0° C. for 1 h. Then 4,6-dichloro-1-methyl-1H-pyrrolo[2,3-b]pyridine (386 mg, 1.92 mmol, 1.2 eq) was added to above mixture and the resulting mixture was stirred for 1 h. The mixture was quenched with saturated aqueous NH₄Cl solution (20 mL) and extracted with EA (30 mL×3). The organic layer was washed with brine (30 mL×4), dried over anhydrous sodium sulfate, filtered and concentrated under reduced pressure. The residue was purified by flash column chromatography on silica gel (EA/PE=1/5 v/v) to give tert-butyl (R)-3-((6-chloro-1-methyl-1H-pyrrolo[2,3-b]pyridin-4-yl)oxy) pyrrolidine-1-carboxylate (128 mg, yield 23%) as a white solid. LC-MS (ESI): mass calcd. for C₁₇H₂₂ClN₃O₃, 351.13; m/z found, 352.2 [M+H]⁺.

### Step C: tert-butyl (R)-3-((6-(2-((S)-1-amino-5-(tert-butoxy)-1,5-dioxopentan-2-yl)-1-oxoisoindolin-5-yl)-1-methyl-1H-pyrrolo[2,3-b]pyridin-4-yl)oxy) pyrrolidine-1-carboxylate

A mixture of tert-butyl (R)-3-((6-chloro-1-methyl-1H-pyrrolo[2,3-b]pyridin-4-yl)oxy) pyrrolidine-1-carboxylate (128 mg, 364 μmol, 1.0 eq), tert-butyl(S)-5-amino-5-oxo-4-(1-oxo-5-(4,4,5,5-tetramethyl-1,3,2-dioxaborolan-2-yl) isoindolin-2-yl) pentanoate (Intermediate 33, 210 mg, 473 μmol, 1.3 eq), 1,1′-Bis(di-t-butylphosphino) ferrocene palladium dichloride (23.7 mg, 36.4 μmol, 0.1 eq), and Potassium phosphate tribasic (232 mg, 1.09 mmol, 3.0 eq) in 1,4-Dioxane (6.00 mL) and H₂O (0.60 mL) was stirred under N₂ at 90° C. for 2 h. After cooled to room temperature, the reaction mixture was diluted with DCM (50 mL) and filtered. The filtrate was concentrated under reduced pressure and the residue was purified by Prep-TLC(100% EA) to give tert-butyl (R)-3-((6-(2-((S)-1-amino-5-(tert-butoxy)-1,5-dioxopentan-2-yl)-1-oxoisoindolin-5-yl)-1-methyl-1H-pyrrolo[2,3-b]pyridin-4-yl)oxy) pyrrolidine-1-carboxylate (138 mg, yield 60%) as a yellow oil. LC-MS (ESI): mass calcd. for C₃₄H₄₃N₅O₇, 633.32; m/z found, 634.3 [M+H]⁺

### Step D: (S)-3-(5-(1-methyl-4-(((R)-pyrrolidin-3-yl)oxy)-1H-pyrrolo[2,3-b]pyridin-6-yl)-1-oxoisoindolin-2-yl) piperidine-2,6-dione

To a solution of tert-butyl (R)-3-((6-(2-((S)-1-amino-5-(tert-butoxy)-1,5-dioxopentan-2-yl)-1-oxoisoindolin-5-yl)-1-methyl-1H-pyrrolo[2,3-b]pyridin-4-yl)oxy) pyrrolidine-1-carboxylate (50.0 mg, 78.9 μmol, 1.0 eq) in MeCN (10.0 mL) was added anhydrous benzenesulfonic acid (37.4 mg, 237 μmol, 3.0 eq) under room temperature. The mixture was stirred under 85° C. for 16 h. After evaporation, the crude product was purified by Prep-HPLC with YMC-Actus Triart C18 (5 μm, 21.2×250 mm), and mobile phase of 5-95% ACN in water (0.1% FA) over 23 min and then hold at 95% ACN for 3 min, at a flow rate of 20 mL/min to give(S)-3-(5-(1-methyl-4-(((R)-pyrrolidin-3-yl)oxy)-1H-pyrrolo[2,3-b]pyridin-6-yl)-1-oxoisoindolin-2-yl) piperidine-2,6-dione formate (5.00 mg, yield 14%) as a white solid. LC-MS (ESI): mass calcd. for C₂₅H₂₅N₅O₄, 459.19; m/z found, 460.2 [M+H]⁺. ¹H NMR (400 MHZ, DMSO-d₆) δ 11.00 (s, 1H), 8.42 (s, 1H), 8.36 (d, J=8.0 Hz, 1H), 8.27 (s, 1H), 7.83 (d, J=8.0 Hz, 1H), 7.42 (d, J=3.4 Hz, 1H), 7.34 (s, 1H), 6.48 (d, J=3.4 Hz, 1H), 5.50-5.48 (m, 1H), 5.18-5.13 (m, 1H), 4.57 (d, J=17.2 Hz, 1H), 4.44 (d, J=17.2 Hz, 1H), 3.87 (s, 3H), 3.26 (d, J=12.6 Hz, 2H), 3.16 (dd, J=15.4, 7.8 Hz, 2H), 2.97-2.89 (m, 1H), 2.63 (dd, J=13.0, 2.6 Hz, 1H), 2.46-2.37 (m, 1H), 2.29-2.24 (m, 1H), 2.13-2.01 (m, 2H).

### Example 220:3-(5-(7-(1,1-dioxidothietan-3-yl)-4-(pyrrolidin-1-ylmethyl)-7H-pyrrolo[2,3-d]pyrimidin-2-yl)-1-oxoisoindolin-2-yl) piperidine-2,6-dione

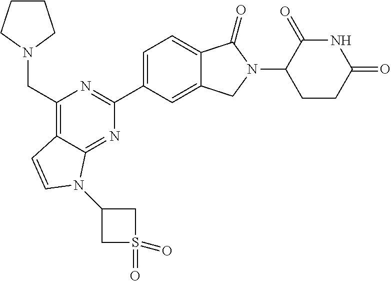 [SMILES: O=C1CCC(N2Cc3cc(-c4nc(CN5CCCC5)c5ccn(C6CS(=O)(=O)C6)c5n4)ccc3C2=O)C(=O)N1]

The title compound was prepared in a manner analogous to Example 203 by alkylation of chloro-4-(pyrrolidin-1-ylmethyl)-7H-pyrrolo[2,3-d]pyrimidine (Intermediate 78) with 3-bromothietane 1,1-dioxide followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid (TFA salt). LC-MS (ESI): mass calced for: C₂₇H₂₈N₆O₅S, 548.18; m/z found, 549.1 [M+H]⁺. ¹H NMR (400 MHZ, DMSO-d₆) δ 11.03 (s, 1H), 10.35 (s, 1H), 8.88-8.80 (m, 2H), 7.99 (d, J=3.6 Hz, 1H), 7.91 (d, J=8.0 Hz, 1H), 6.90 (d, J=3.6 Hz, 1H), 5.89-5.79 (m, 1H), 5.18 (dd, J=13.2, 5.2 Hz, 1H), 5.04 (d, J=5.6 Hz, 2H), 4.97 (d, J=8.0 Hz, 4H), 4.60 (d, J=17.4 Hz, 1H), 4.47 (d, J=17.6 Hz, 1H), 3.80 (s, 2H), 3.27 (s, 2H), 3.00-2.89 (m, 1H), 2.63 (d, J=18.2 Hz, 1H), 2.44 (d, J=13.0 Hz, 1H), 2.11-2.03 (m, 5H). ¹⁹F NMR (400 MHZ, DMSO-d₆) δ-73.67 (ppm).

### Example 221:3-(5-(5-chloro-4-(pyrrolidin-1-ylmethyl)-1H-pyrrolo[2,3-b]pyridin-6-yl)-1-oxoisoindolin-2-yl) piperidine-2,6-dione

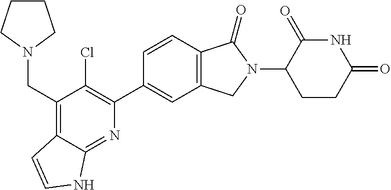 [SMILES: O=C1CCC(N2Cc3cc(-c4nc5[nH]ccc5c(CN5CCCC5)c4Cl)ccc3C2=O)C(=O)N1]

The title compound was prepared in a manner analogous to Example 1 by Suzuki coupling of 5,6-dichloro-4-(pyrrolidin-1-ylmethyl)-1H-pyrrolo[2,3-b]pyridine (Intermediate 79) with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₅H₂₄ClN₅O₃, 477.95; m/z found, 478.1 [M+H]⁺. ¹H NMR (400 MHZ, DMSO-d₆) δ 11.90 (s, 1H), 11.02 (s, 1H), 8.14 (s, 1H), 7.84 (s, 1H), 7.82 (d, J=7.8 Hz, 1H), 7.74 (d, J=7.8 Hz, 1H), 7.61-7.58 (m, 1H), 6.75 (dd, J=3.0, 1.8 Hz, 1H), 5.19-5.14 (m, 1H), 4.55 (d, J=17.4 Hz, 1H), 4.41 (d, J=17.4 Hz, 1H), 4.09 (s, 2H), 2.98-2.88 (m, 1H), 2.62 (d, J=12.0 Hz, 5H), 2.46-2.37 (m, 1H), 2.08-2.00 (m, 1H), 1.71 (s, 4H).

### Example 222:3-(5-(1-(1,1-dioxidothietan-3-yl)-4-(pyrrolidin-1-ylmethyl)-1H-pyrrolo[2,3-b]pyridin-6-yl)-4-fluoro-1-oxoisoindolin-2-yl) piperidine-2,6-dione

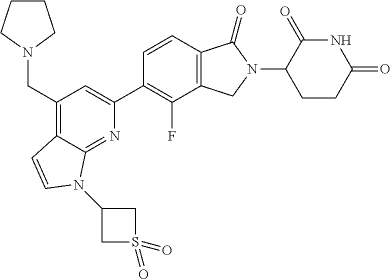 [SMILES: O=C1CCC(N2Cc3c(ccc(-c4cc(CN5CCCC5)c5ccn(C6CS(=O)(=O)C6)c5n4)c3F)C2=O)C(=O)N1]

The title compound was prepared in a manner analogous to Example 1 by reductive amination between pyrrolidine and 6-chloro-1-(1,1-dioxidothietan-3-yl)-1H-pyrrolo[2,3-b]pyridine-4-carbaldehyde (Intermediate 35) followed by Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14) as a white solid. LC-MS (ESI): mass calced for: C₂₈H₂₈FN₅O₅S, 565.62; m/z found, 566.2 [M+H]⁺. ¹H NMR (400 MHZ, DMSO-d₆) δ 11.03 (s, 1H), 8.28 (t, J=7.2 Hz, 1H), 8.14 (s, 1H), 7.89 (d, J=3.6 Hz, 1H), 7.70 (d, J=8.0 Hz, 2H), 6.79 (d, J=3.6 Hz, 1H), 5.82-5.74 (m, 1H), 5.19-5.14 (m, 1H), 4.89 (d, J=7.4 Hz, 4H), 4.66 (d, J=17.4 Hz, 1H), 4.50 (d, J=17.4 Hz, 1H), 3.99 (s, 2H), 3.00-2.89 (m, 1H), 2.63 (d, J=16.6 Hz, 1H), 2.57 (s, 4H), 2.49-2.41 (m, 1H), 2.07-2.03 (m, 1H), 1.74 (s, 4H). ¹⁹F NMR (400 MHZ, DMSO-d₆) δ-123.93 (ppm).

### Example 223:3-(4-fluoro-5-(1-(oxetan-3-yl)-4-(pyrrolidin-1-ylmethyl)-1H-pyrrolo[2,3-b]pyridin-6-yl)-1-oxoisoindolin-2-yl) piperidine-2,6-dione

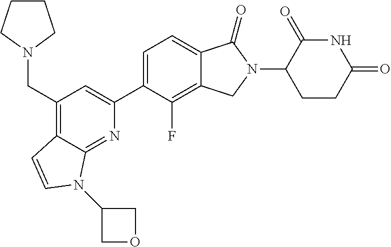 [SMILES: O=C1CCC(N2Cc3c(ccc(-c4cc(CN5CCCC5)c5ccn(C6COC6)c5n4)c3F)C2=O)C(=O)N1]

The title compound was prepared in a manner analogous to Example 1 by reductive amination between pyrrolidine and 6-chloro-1-(oxetan-3-yl)-1H-pyrrolo[2,3-b]pyridine-4-carbaldehyde (Intermediate 29) followed by Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14) as a yellow solid. LC-MS (ESI): mass calced for: C₂₈H₂₈FN504, 517.2; m/z found, 518.2 [M+H]⁺. ¹H NMR (400 MHZ, DMSO-d₆) δ 11.04 (s, 1H), 8.22 (t, J=7.2 Hz, 1H), 8.08 (s, 1H), 7.73 (d, J=7.8 Hz, 2H), 6.84 (s, 1H), 6.09-6.05 (m, 1H), 5.17 (dd, J=13.2, 5.2 Hz, 1H), 5.06 (d, J=7.2 Hz, 4H), 4.67 (d, J=17.4 Hz, 1H), 4.50 (d, J=17.4 Hz, 1H), 3.99 (s, 2H), 3.29 (d, J=2.2 Hz, 4H), 2.92 (dd, J=13.2, 5.0 Hz, 1H), 2.61 (s, 1H), 2.47-2.41 (m, 1H), 2.09-2.02 (m, 1H), 1.85 (s, 4H).

### Example 224:3-(5-(3-cyclobutyl-7-(pyrrolidin-1-ylmethyl)-1H-pyrazolo[4,3-b]pyridin-5-yl)-1-oxoisoindolin-2-yl) piperidine-2,6-dione

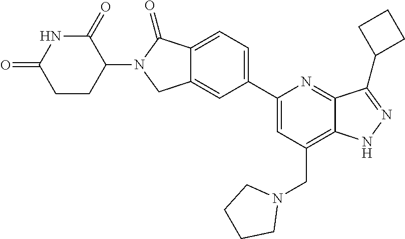 [SMILES: O=C1CCC(N2Cc3cc(-c4cc(CN5CCCC5)c5[nH]nc(C6CCC6)c5n4)ccc3C2=O)C(=O)N1]

### Step A: 5-chloro-3-cyclobutyl-7-(pyrrolidin-1-ylmethyl)-1-(tetrahydro-2H-pyran-2-yl)-1H-pyrazolo[4,3-b]pyridine

To a solution of 5-chloro-3-cyclobutyl-1-(tetrahydro-2H-pyran-2-yl)-1H-pyrazolo[4,3-b]pyridine-7-carbaldehyde (Intermediate 80, 90 mg, 281 μmol, 1.0 eq) in DCM (5.0 mL) were added pyrrolidine (60 mg, 844 μmol, 3.0 eq) and AcOH (33.8 mg, 563 μmol, 2.0 eq) at 25° C. The reaction mixture was stirred at room temperature for 15 h. Then NaBH(OAc) 3 (89.5 mg, 422 μmol, 1.5 eq) was added to above mixture and the resulting reaction mixture was stirred at room temperature for 1 h. The reaction mixture was quenched with water (10 mL) and extracted with DCM (10 mL×3). The organic layer was washed with brine (10 mL), dried over anhydrous Na₂SO₄, filtered and concentrated under reduced pressure. The residue was purified by flash column chromatography on silica gel (PE/EA=1/1 v/v) to give 5-chloro-3-cyclobutyl-7-(pyrrolidin-1-ylmethyl)-1-(tetrahydro-2H-pyran-2-yl)-1H-pyrazolo[4,3-b]pyridine (30 mg, yield 28%) as a yellow solid. LC-MS (ESI): mass calcd. for C₂₀H₂₇ClN₄O, 374.2; m/z found, 375.2 [M+H]⁺.

### Step B: 3-(5-(3-cyclobutyl-7-(pyrrolidin-1-ylmethyl)-1-(tetrahydro-2H-pyran-2-yl)-1H-pyrazolo[4,3-b]pyridin-5-yl)-1-oxoisoindolin-2-yl) piperidine-2,6-dione

To a solution of 5-chloro-3-cyclobutyl-7-(pyrrolidin-1-ylmethyl)-1-(tetrahydro-2H-pyran-2-yl)-1H-pyrazolo[4,3-b]pyridine (30 mg, 80 μmol, 1.0 eq) and 3-(1-oxo-5-(4,4,5,5-tetramethyl-1,3,2-dioxaborolan-2-yl) isoindolin-2-yl) piperidine-2,6-dione (Intermediate 13, 44.4 mg, 120 μmol, 1.5 eq) in 1,4-Dioxane (3.0 mL) and H₂O (0.2 mL) were added K₃PO₄ (51 mg, 240 μmol, 3.0 eq) and Pd (dtbpf) Cl₂ (10.4 mg, 16 μmol, 0.2 eq) at 25° C. The reaction mixture was stirred under N₂ at 95° C. for 1 h. After cooled to room temperature, the reaction mixture was filtered and the filtrate was concentrated under reduced pressure. The residue was purified by Prep-TLC(DCM/MeOH=10/1 v/v) to give 3-(5-(3-cyclobutyl-7-(pyrrolidin-1-ylmethyl)-1-(tetrahydro-2H-pyran-2-yl)-1H-pyrazolo[4,3-b]pyridin-5-yl)-1-oxoisoindolin-2-yl) piperidine-2,6-dione (30 mg, yield 64%) as a yellow solid. LC-MS (ESI): mass calcd. for C₃₃H₃₈N₆O₄, 582.3; m/z found, 583.3 [M+H]⁺.

### Step C: 3-(5-(3-cyclobutyl-7-(pyrrolidin-1-ylmethyl)-1H-pyrazolo[4,3-b]pyridin-5-yl)-1-oxoisoindolin-2-yl) piperidine-2,6-dione

To a solution of 3-(5-(3-cyclobutyl-7-(pyrrolidin-1-ylmethyl)-1-(tetrahydro-2H-pyran-2-yl)-1H-pyrazolo[4,3-b]pyridin-5-yl)-1-oxoisoindolin-2-yl) piperidine-2,6-dione (30 mg, 51.5 μmol, 1.0 eq) in DCM (3.0 mL) was added TFA (1.0 mL, 13.1 mmol, 255 eq) at 25° C. The reaction mixture was stirred at room temperature for 3 h. After evaporation, the residue was purified by Prep-TLC(DCM/MeOH=10/1 v/v) to afford 3-(5-(3-cyclobutyl-7-(pyrrolidin-1-ylmethyl)-1H-pyrazolo[4,3-b]pyridin-5-yl)-1-oxoisoindolin-2-yl) piperidine-2,6-dione (4.1 mg, yield 16%) as a white solid. LC-MS (ESI): mass calcd. for C₂₈H₃₀N₆O₃, 498.2; m/z found, 499.2 [M+H]⁺. ¹H NMR (400 MHZ, DMSO-d₆) δ 13.05 (s, 1H), 11.08 (s, 1H), 8.41 (s, 1H), 8.36 (d, J=8.0 Hz, 1H), 8.01 (s, 1H), 7.92 (d, J=8.0 Hz, 1H), 5.24-5.20 (m, 1H), 4.64 (d, J=17.4 Hz, 1H), 4.51 (d, J=17.4 Hz, 1H), 4.21-4.11 (m, 1H), 4.07 (s, 2H), 3.05-2.93 (m, 1H), 2.75-2.59 (m, 7H), 2.50-2.45 (m, 3H), 2.21-2.06 (m, 3H), 1.83-1.80 (m, 4H).

### Example 225:3-(5-(3-(1,1-dioxidothietan-3-yl)-7-(pyrrolidin-1-ylmethyl)-3H-imidazo[4,5-b]pyridin-5-yl)-1-oxoisoindolin-2-yl) piperidine-2,6-dione

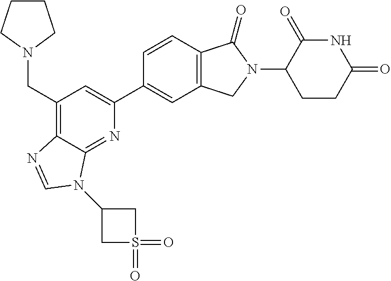 [SMILES: O=C1CCC(N2Cc3cc(-c4cc(CN5CCCC5)c5ncn(C6CS(=O)(=O)C6)c5n4)ccc3C2=O)C(=O)N1]

The title compound was prepared in a manner analogous to Example 203 by alkylation between pyrrolidine and 3-(7-(bromomethyl)-5-chloro-3H-imidazo[4,5-b]pyridin-3-yl) thietane 1,1-dioxide (Intermediate 81) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₇H₂₈N₆O₅S, 548.18; m/z found, 549.2 [M+H]⁺. ¹H NMR (400 MHZ, DMSO-d₆) δ 11.02 (s, 1H), 8.65 (s, 1H), 8.44 (s, 1H), 8.36 (d, J=8.0 Hz, 1H), 8.17 (s, 1H), 8.02 (s, 1H), 7.85 (d, J=8.0 Hz, 1H), 5.70-5.63 (m, 1H), 5.20-5.08 (m, 3H), 4.91 (dd, J=15.0, 9.2 Hz, 2H), 4.51 (d, J=17.2 Hz, 1H), 4.46 (d, J=17.2 Hz, 1H), 4.10 (s, 2H), 2.93 (dd, J=21.8, 9.4 Hz, 1H), 2.65-2.58 (m, 5H), 2.48-2.43 (m, 1H), 2.09-2.01 (m, 1H), 1.75 (s, 4H).

### Example 226:3-(4-fluoro-5-(3-(isopropylamino)-7-(pyrrolidin-1-ylmethyl)-1H-pyrazolo[4,3-b]pyridin-5-yl)-1-oxoisoindolin-2-yl) piperidine-2,6-dione

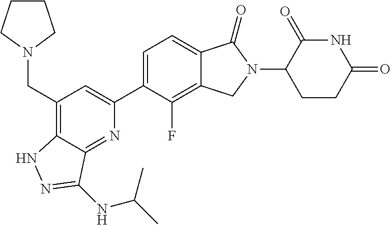 [SMILES: CC(C)Nc1n[nH]c2c(CN3CCCC3)cc(-c3ccc4c(c3F)CN(C3CCC(=O)NC3=O)C4=O)nc12]

The title compound was prepared in a manner analogous to Example 173 by reductive amination between pyrrolidine and 5-chloro-3-(isopropylamino)-1-((2-(trimethylsilyl)ethoxy)methyl)-1H-pyrazolo[4,3-b]pyridine-7-carbaldehyde (Intermediate 25) followed by Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14) followed by de-protection of N-SEM group as a yellow solid. LC-MS (ESI): mass calced for: C₂₇H₃₀FN7O₃, 519.24; m/z found, 520.3 [M+H]⁺. ¹H NMR (400 MHZ, DMSO-d₆) δ 11.84 (s, 1H), 11.04 (s, 1H), 8.19 (t, J=7.4 Hz, 1H), 7.73 (dd, J=15.0, 7.4 Hz, 2H), 5.65 (s, 1H), 5.16 (dd, J=13.2, 5.2 Hz, 1H), 4.66 (d, J=17.4 Hz, 1H), 4.49 (d, J=17.4 Hz, 1H), 4.02-4.00 (m, 1H), 3.38 (d, J=1.2 Hz, 2H), 2.97-2.90 (m, 1H), 2.62 (d, J=17.0 Hz, 5H), 2.46 (s, 1H), 2.05 (s, 1H), 1.76 (s, 4H), 1.26 (d, J=6.4 Hz, 6H). ¹⁹F NMR (400 MHZ, DMSO-d₆) δ-73.42,-124.58 (ppm).

### Example 227:3-(4-chloro-5-(3-(isopropylamino)-7-(pyrrolidin-1-ylmethyl)-1H-pyrazolo[4,3-b]pyridin-5-yl)-1-oxoisoindolin-2-yl) piperidine-2,6-dione

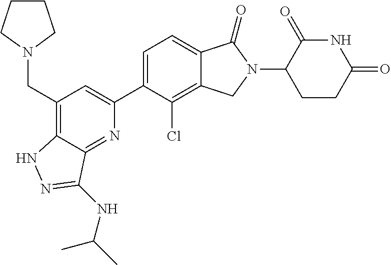 [SMILES: CC(C)Nc1n[nH]c2c(CN3CCCC3)cc(-c3ccc4c(c3Cl)CN(C3CCC(=O)NC3=O)C4=O)nc12]

The title compound was prepared in a manner analogous to Example 173 by reductive amination between pyrrolidine and 5-chloro-3-(isopropylamino)-1-((2-(trimethylsilyl)ethoxy)methyl)-1H-pyrazolo[4,3-b]pyridine-7-carbaldehyde (Intermediate 25) followed by Suzuki coupling with 3-(4-chloro-1-oxo-5-(4,4,5,5-tetramethyl-1,3,2-dioxaborolan-2-yl) isoindolin-2-yl) piperidine-2,6-dione (Intermediate 32) as a yellow solid. LC-MS (ESI): mass calced for: C₂₇H₃₀ClN₇O₃, 535.21; m/z found, 536.21 [M+H]⁺. ¹H NMR (400 MHZ, DMSO-d₆) δ 11.84 (s, 1H), 11.04 (s, 1H), 8.28 (s, 1H), 7.84-7.78 (m, 2H), 7.54 (s, 1H), 5.57 (d, J=8.0 Hz, 1H), 5.21-5.16 (m, 1H), 4.57 (d, J=17.8 Hz, 1H), 4.41 (d, J=17.8 Hz, 1H), 4.01-3.96 (m, 1H), 3.90 (s, 2H), 3.00-2.89 (m, 1H), 2.62 (d, J=17.6 Hz, 1H), 2.56 (s, 4H), 2.46 (s, 1H), 2.11-2.01 (m, 1H), 1.74 (s, 4H), 1.24 (d, J=6.4 Hz, 6H).

### Example 228:3-(4-chloro-5-(1-(oxetan-3-yl)-4-(pyrrolidin-1-ylmethyl)-1H-pyrrolo[2,3-b]pyridin-6-yl)-1-oxoisoindolin-2-yl) piperidine-2,6-dione

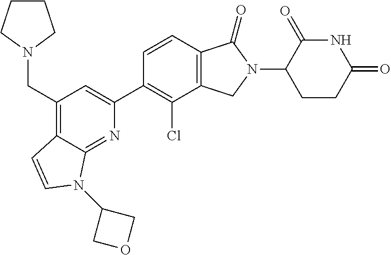 [SMILES: O=C1CCC(N2Cc3c(ccc(-c4cc(CN5CCCC5)c5ccn(C6COC6)c5n4)c3Cl)C2=O)C(=O)N1]

The title compound was prepared in a manner analogous to Example 1 by reductive amination between pyrrolidine and 6-chloro-1-(oxetan-3-yl)-1H-pyrrolo[2,3-b]pyridine-4-carbaldehyde (Intermediate 29) followed by Suzuki coupling with 3-(4-chloro-1-oxo-5-(4,4,5,5-tetramethyl-1,3,2-dioxaborolan-2-yl) isoindolin-2-yl) piperidine-2,6-dione (Intermediate 32) as a white solid. LC-MS (ESI): mass calcd. for C₂₈H₂₈ClN₅O₄, 533.18; m/z found, 534.5 [M+H]⁺. ¹H NMR (400 MHZ, DMSO-d₆) δ 11.04 (s, 1H), 8.00 (d, J=3.6 Hz, 1H), 7.83 (s, 2H), 7.46 (s, 1H), 6.79 (d, J=3.6 Hz, 1H), 6.04-5.93 (m, 1H), 5.21-5.15 (m, 1H), 5.06-5.00 (m, 4H), 4.57 (d, J=18.0 Hz, 1H), 4.41 (d, J=18.0 Hz, 1H), 3.96 (s, 2H), 2.97-2.92 (m, 1H), 2.65-2.59 (m, 1H), 2.56-2.53 (m, 4H), 2.46-2.39 (m, 1H), 2.08-2.03 (m, 1H), 1.75-1.69 (m, 4H).

### Example 229:3-(5-(5-chloro-1-methyl-4-(pyrrolidin-1-ylmethyl)-1H-pyrrolo[2,3-b]pyridin-6-yl)-1-oxoisoindolin-2-yl) piperidine-2,6-dione

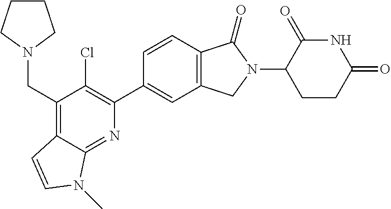 [SMILES: Cn1ccc2c(CN3CCCC3)c(Cl)c(-c3ccc4c(c3)CN(C3CCC(=O)NC3=O)C4=O)nc21]

The title compound was prepared in a manner analogous to Example 1 by reductive amination between pyrrolidine and 5,6-dichloro-1-methyl-1H-pyrrolo[2,3-b]pyridine-4-carbaldehyde (Intermediate 107) followed Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₆H₂₆ClN₅O₃, 491.98; m/z found, 492.1 [M+H]⁺. ¹H NMR (400 MHZ, DMSO-d₆) δ 11.02 (s, 1H), 8.07 (s, 1H), 7.86-7.81 (m, 2H), 7.74 (d, J=9.0 Hz, 1H), 7.64 (d, J=3.4 Hz, 1H), 6.77 (d, J=3.4 Hz, 1H), 5.19-5.14 (m, 1H), 4.56 (d, J=17.4 Hz, 1H), 4.42 (d, J=17.4 Hz, 1H), 4.09 (s, 2H), 3.81 (s, 3H), 2.97-2.89 (m, 1H), 2.66 (dd, J=11.2, 2.4 Hz, 1H), 2.63-2.55 (m, 4H), 2.43 (dd, J=12.8, 4.4 Hz, 1H), 2.09-2.03 (m, 1H), 1.72-1.68 (m, 4H).

### Example 230:3-(4-chloro-5-(1-(1,1-dioxidothietan-3-yl)-4-(pyrrolidin-1-ylmethyl)-1H-pyrrolo[2,3-b]pyridin-6-yl)-1-oxoisoindolin-2-yl) piperidine-2,6-dione

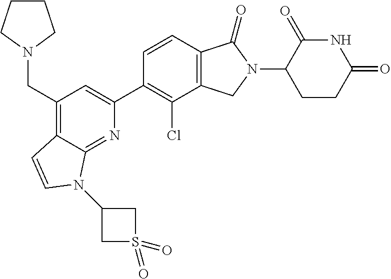 [SMILES: O=C1CCC(N2Cc3c(ccc(-c4cc(CN5CCCC5)c5ccn(C6CS(=O)(=O)C6)c5n4)c3Cl)C2=O)C(=O)N1]

The title compound was prepared in a manner analogous to Example 1 by reductive amination between pyrrolidine and 6-chloro-1-(1,1-dioxidothietan-3-yl)-1H-pyrrolo[2,3-b]pyridine-4-carbaldehyde (Intermediate 35) followed by Suzuki coupling with 3-(4-chloro-1-oxo-5-(4,4,5,5-tetramethyl-1,3,2-dioxaborolan-2-yl) isoindolin-2-yl) piperidine-2,6-dione (Intermediate 32) as a white solid. LC-MS (ESI): mass calced for: C₂₈H₂₈ClN₅O₅S, 582.07; m/z found, 582.2 [M+H]⁺. ¹H NMR (400 MHZ, DMSO-d₆) δ 11.04 (s, 1H), 7.90 (d, J=3.6 Hz, 1H), 7.87 (d, J=7.8 Hz, 1H), 7.83 (d, J=7.8 Hz, 1H), 7.52 (s, 1H), 6.80 (d, J=3.6 Hz, 1H), 5.75-5.67 (m, 1H), 5.20-5.16 (m, 1H), 4.85 (d, J=7.4 Hz, 4H), 4.57 (d, J=17.6 Hz, 1H), 4.41 (d, J=17.6 Hz, 1H), 4.00 (s, 2H), 3.00-2.88 (m, 1H), 2.69-2.52 (m, 5H), 2.46 (d, J=4.2 Hz, 1H), 2.09-2.04 (m, 1H), 1.74 (s, 4H).

### Example 231: (3S)-3-(5-(1-methyl-4-(piperidin-2-yl)-1H-pyrrolo[2,3-b]pyridin-6-yl)-1-oxoisoindolin-2-yl) piperidine-2,6-dione

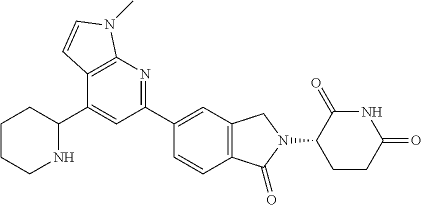 [SMILES: Cn1ccc2c(C3CCCCN3)cc(-c3ccc4c(c3)CN([C@H]3CCC(=O)NC3=O)C4=O)nc21]

### Step A: 4,6-dichloro-1-methyl-1H-pyrrolo[2,3-b]pyridine

To a solution of 4,6-dichloro-1H-pyrrolo[2,3-b]pyridine (4.00 g, 21.4 mmol, 1.0 eq) in DMF (40.0 mL) was added NaH (60% suspend in oil) (1.28 g, 32.1 mmol, 1.5 eq) at 0° C. and the mixture was stirred for 20 min. Then MeI (4.55 g, 2.01 mL, 32.1 mmol, 1.5 eq) was added to above mixture and the resulting reaction mixture was stirred at room temperature for 2 h. The mixture was quenched with saturated aqueous NH₄Cl solution (40 mL) and extract with ethyl acetate (30 mL×3). The organic layer was washed with brine (30 mL×4), dried over anhydrous sodium sulfate, filter and concentrate under reduced pressure. The residue was purified by flash column chromatography on silica gel (PE/EtOAc=3/1 v/v) to give 4,6-dichloro-1-methyl-1H-pyrrolo[2,3-b]pyridine (3.70 g, yield 86%) as a yellow oil. LC-MS (ESI): mass calced for C₈H₆C₁₂N₂:200; m/z found, 201.1 [M+H]⁺

### Step B: (1-(tert-butoxycarbonyl)-4,4-dimethylpiperidin-2-yl) zinc (II) chloride

To a solution of tert-butyl piperidine-1-carboxylate (4.00 g, 21.6 mmol, 1.0 eq) and TMEDA (3.01 g, 3.88 mL, 25.9 mmol, 1.2 eq) in THF (50.0 mL) was added dropwise sec-butyllithium (1.4 M in THF) (23.1 mL, 32.4 mmol, 1.5 eq) under N₂ at −70° C. The reaction mixture was stirred under N₂ at −70° C. for 3 h. Then zinc (II) chloride (0.7 M in THF) (46.3 mL, 32.4 mmol, 1.5 eq) was added dropwise to above mixture and the resulting mixture was stirred at −70° C. for 30 min. Then the reaction mixture was stirred under N₂ at room temperature for 1 h. The solution was directly used in next step without further purification.

### Step C: tert-butyl 2-(6-chloro-1-methyl-1H-pyrrolo[2,3-b]pyridin-4-yl) piperidine-1-carboxylate

To a solution of 4,6-dichloro-1-methyl-1H-pyrrolo[2,3-b]pyridine (2.00 g, 9.95 mmol, 1.0 eq) in THF (30.0 mL) were added tri-tert-butylphosphonium tetrafluoroborate (1.15 g, 3.98 mmol, 0.4 eq), (1-(tert-butoxycarbonyl) piperidin-2-yl) zinc (II) chloride solution (Step B) (2.84 g, 9.95 mmol, 1.1 eq), and diacetoxypalladium (447 mg, 1.99 mmol, 0.2 eq). The reaction mixture was stirred under N₂ at room temperature for 16 h. The mixture was quenched with NH₄OH (30 mL) and extract with ethyl acetate (40 mL×3). The organic layer was washed with brine (30 mL), dried over anhydrous sodium sulfate, filter and concentrate under reduced pressure. The residue was purified by flash column chromatography on silica gel (PE/EtOAc=2/1 v/v) to give tert-butyl 2-(6-chloro-1-methyl-1H-pyrrolo[2,3-b]pyridin-4-yl) piperidine-1-carboxylate (320 mg, yield 9%) as a light yellow oil. LC-MS (ESI): mass calced for C₁₈H₂₄ClN₃O₂, 349; m/z found, 350.2 [M+H]⁺. ¹H NMR (400 MHZ, CDCl₃) δ 7.12 (d, J=3.6 Hz, 1H), 6.91 (s, 1H), 6.54 (s, 1H), 4.30 (s, 1H), 4.22-4.04 (m, 1H), 3.85 (s, 3H), 3.16-3.00 (m, 1H), 2.96-2.75 (m, 2H), 2.10 (d, J=10.8 Hz, 1H), 1.86-1.74 (m, 2H), 1.61 (d, J=20.4 Hz, 1H), 1.47 (d, J=9.8 Hz, 9H).

### Step D: tert-butyl 2-(6-(2-((S)-1-amino-5-(tert-butoxy)-1,5-dioxopentan-2-yl)-1-oxoisoindolin-5-yl)-1-methyl-1H-pyrrolo[2,3-b]pyridin-4-yl) piperidine-1-carboxylate

To a solution of tert-butyl 2-(6-chloro-1-methyl-1H-pyrrolo[2,3-b]pyridin-4-yl) piperidine-1-carboxylate (100 mg, 286 μmol, 1.0 eq) in 1,4-dioxane (4.00 mL) and H₂O (0.40 mL) were added tert-butyl(S)-5-amino-5-oxo-4-(1-oxo-5-(4,4,5,5-tetramethyl-1,3,2-dioxaborolan-2-yl) isoindolin-2-yl) pentanoate (Intermediate 33, 165 mg, 372 μmol, 1.3 eq), Potassium phosphate tribasic (182 mg, 857 μmol, 3.0 eq), and 1,1′-Bis(di-t-butylphosphino) ferrocene palladium dichloride (18.6 mg, 28.6 μmol, 0.1 eq). The reaction mixture was stirred under N₂ at 95° C. for 2 h. After cooled to room temperature, the mixture was diluted with H₂O (5 mL) and extract with ethyl acetate (10 mL×3). The organic layer was washed with brine (10 mL), dried over anhydrous sodium sulfate, filtered and concentrate under reduced pressure. The residue was purified by flash column chromatography on silica gel (PE/EtOAc=1/1 v/v) to give tert-butyl 2-(6-(2-((S)-1-amino-5-(tert-butoxy)-1,5-dioxopentan-2-yl)-1-oxoisoindolin-5-yl)-1-methyl-1H-pyrrolo[2,3-b]pyridin-4-yl) piperidine-1-carboxylate (80.0 mg, yield 44%) as a light yellow oil. LC-MS (ESI): mass calced for C₃₅H₄₅N₅O₆, 631; m/z found, 632.1 [M+H]⁺.

### Step E: (3S)-3-(5-(1-methyl-4-(piperidin-2-yl)-1H-pyrrolo[2,3-b]pyridin-6-yl)-1-oxoisoindolin-2-yl) piperidine-2,6-dione

To a stirred solution of tert-butyl 2-(6-(2-((S)-1-amino-5-(tert-butoxy)-1,5-dioxopentan-2-yl)-1-oxoisoindolin-5-yl)-1-methyl-1H-pyrrolo[2,3-b]pyridin-4-yl) piperidine-1-carboxylate (80.0 mg, 127 μmol, 1.0 eq) in ACN (10.0 mL) was added benzenesulfonic acid (60.0 mg, 380 μmol, 3.0 eq). The reaction mixture was stirred under nitrogen atmosphere at 95° C. for 2 h. After evaporation, the residue was purified by Prep-HPLC with YMC-Actus Triart 18C(5 μm, 20×250 mm), and mobile phase of 5-99% ACN in water (0.1% TFA) over 10 min and then hold at 100% ACN for 2 min, at a flow rate of 25 mL/min to give (3S)-3-(5-(1-methyl-4-(piperidin-2-yl)-1H-pyrrolo[2,3-b]pyridin-6-yl)-1-oxoisoindolin-2-yl) piperidine-2,6-dione trifluoroacetate (23.0 mg, yield 40%) as a light yellow solid. LC-MS (ESI): mass calced for C₂₆H₂₇N₅O₃, 457; m/z found, 458.3 [M+H]⁺. ¹H NMR (400 MHZ, DMSO-d₆) δ 11.02 (s, 1H), 8.92 (d, J=11.2 Hz, 1H), 8.58 (s, 1H), 8.42 (s, 1H), 8.37 (d, J=8.0 Hz, 1H), 7.86 (d, J=8.0 Hz, 1H), 7.75 (s, 1H), 7.62 (dd, J=11.0, 3.6 Hz, 1H), 6.65 (d, J=3.6 Hz, 1H), 5.18-5.13 (m, 1H), 4.66-4.52 (m, 1H), 4.45 (d, J=17.2 Hz, 1H), 3.91 (s, 3H), 3.48-3.37 (m, 4H), 3.08-2.92 (m, 2H), 2.63 (d, J=16.6 Hz, 1H), 2.49-2.42 (m, 1H), 2.11-1.78 (m, 5H). 19F NMR (400 MHZ, DMSO-d₆) δ-73.84 (ppm).

### Example 232:3-(5-(8-isopropoxy-4-(pyrrolidin-1-ylmethyl)-1,5-naphthyridin-2-yl)-1-oxoisoindolin-2-yl) piperidine-2,6-dione

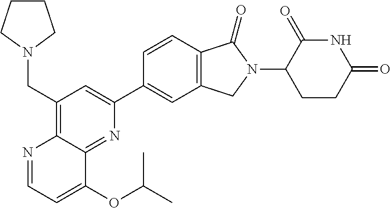 [SMILES: CC(C)Oc1ccnc2c(CN3CCCC3)cc(-c3ccc4c(c3)CN(C3CCC(=O)NC3=O)C4=O)nc12]

The title compound was prepared in a manner analogous to Example 203 by alkylation between pyrrolidine and 4-(bromomethyl)-2-chloro-8-isopropoxy-1,5-naphthyridine (Intermediate 82) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₉H₃₁N₅O₄, 513.6; m/z found, 514.2 [M+H]⁺. ¹H NMR (400 MHz, DMSO-d₆) δ 11.00 (s, 1H), 8.79 (d, J=5.2 Hz, 1H), 8.44 (s, 2H), 8.40 (d, J=8.0 Hz, 1H), 8.15 (s, 1H), 7.93 (d, J=8.0 Hz, 1H), 7.33 (d, J=3.8 Hz, 1H), 5.20-5.15 (m, 1H), 5.08-5.02 (m, 1H), 4.63 (d, J=17.4 Hz, 1H), 4.49 (d, J=17.4 Hz, 1H), 4.47 (s, 2H), 2.99-2.90 (m, 1H), 2.81 (s, 4H), 2.64 (d, J=18.6 Hz, 1H), 2.49-2.38 (m, 1H), 2.10-2.03 (m, 1H), 1.84 (s, 4H), 1.48 (d, J=6.0 Hz, 6H).

### Example 233:3-(1-ox0-5-(7-oxo-4-(pyrrolidin-1-ylmethyl)-6,7-dihydro-5H-pyrrolo[3,4-b]pyridin-2-yl) isoindolin-2-yl) piperidine-2,6-dione

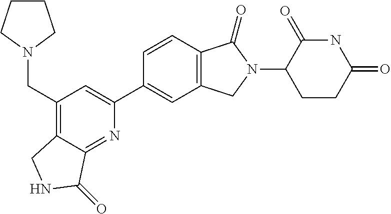 [SMILES: O=C1CCC(N2Cc3cc(-c4cc(CN5CCCC5)c5c(n4)C(=O)NC5)ccc3C2=O)C(=O)N1]

The title compound was prepared in a manner analogous to Example 1 by reductive amination between 4-methoxypiperidine and 2-chloro-7-oxo-6,7-dihydro-5H-pyrrolo[3,4-b]pyridine-4-carbaldehyde (Intermediate 83) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₅H₂₅N₅O₄, 459.19; m/z found, 460.2 [M+H]⁺. ¹H NMR (400 MHZ, DMSO-d₆) δ 11.04 (s, 1H), 10.32 (s, 1H), 9.30 (s, 1H), 8.02 (s, 1H), 7.97-7.88 (m, 3H), 5.20-5.15 (m, 1H), 4.73 (s, 2H), 4.69 (s, 2H), 4.58 (d, J=17.6 Hz, 1H), 4.45 (d, J=17.6 Hz, 1H), 3.57 (s, 2H), 3.19 (d, J=11.2 Hz, 2H), 2.99-2.90 (m, 1H), 2.64 (d, J=15.6 Hz, 1H), 2.46-2.42 (m, 1H), 2.08 (s, 3H), 1.95 (s, 2H). 19F NMR (400 MHZ, DMSO-d₆) δ-73.53 (ppm).

### Example 234:3-(5-(3-amino-1-(oxetan-3-yl)-7-(pyrrolidin-1-ylmethyl)-1H-pyrazolo[4,3-b]pyridin-5-yl)-1-oxoisoindolin-2-yl) piperidine-2,6-dione

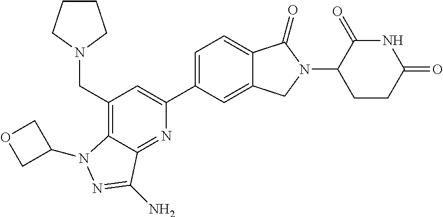 [SMILES: Nc1nn(C2COC2)c2c(CN3CCCC3)cc(-c3ccc4c(c3)CN(C3CCC(=O)NC3=O)C4=O)nc12]

The title compound was prepared in a manner analogous to Example 1 by Suzuki coupling of 5-chloro-1-(oxetan-3-yl)-7-(pyrrolidin-1-ylmethyl)-1H-pyrazolo[4,3-b]pyridin-3-amine (Intermediate 84) with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calcd. for C₂₇H₂₉N₇O₄, 515.2; m/z found, 516.2 [M+H]⁺. ¹H NMR (400 MHZ, DMSO-d₆) δ 11.01 (s, 1H), 8.38 (s, 1H), 8.34 (d, J=8.0 Hz, 1H), 8.07 (s, 1H), 7.92 (s, 1H), 7.83 (d, J=8.0 Hz, 1H), 6.08 (dd, J=14.0, 7.2 Hz, 1H), 5.81 (s, 2H), 5.15 (dd, J=13.2, 5.2 Hz, 1H), 5.02 (t, J=6.4 Hz, 2H), 4.86 (t, J=6.8 Hz, 2H), 4.55 (d, J=17.6 Hz, 1H), 4.43 (d, J=17.6 Hz, 1H), 3.96 (s, 2H), 2.93 (dd, J=8.8, 6.0 Hz, 1H), 2.66-2.59 (m, 1H), 2.47-2.44 (m, 5H), 2.03 (s, 1H), 1.71 (s, 4H).

### Example 235:3-(5-(3-methoxy-7-(pyrrolidin-1-ylmethyl)-1H-pyrazolo[4,3-b]pyridin-5-yl)-1-oxoisoindolin-2-yl) piperidine-2,6-dione

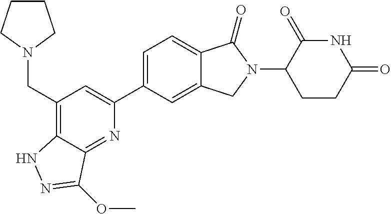 [SMILES: COc1n[nH]c2c(CN3CCCC3)cc(-c3ccc4c(c3)CN(C3CCC(=O)NC3=O)C4=O)nc12]

The title compound was prepared in a manner analogous to Example 1 by reductive amination between pyrrolidine and 5-chloro-3-methoxy-1-(tetrahydro-2H-pyran-2-yl)-1H-pyrazolo[4,3-b]pyridine-7-carbaldehyde (Intermediate 85) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of N-THP group as a white solid. LC-MS (ESI): mass calced for: C₂₅H₂₆N₆O₄, 474.2; m/z found, 475.2 [M+H]⁺. ¹H NMR (400 MHZ, DMSO-d₆) δ 12.25 (s, 1H), 11.01 (s, 1H), 8.33 (s, 1H), 8.24 (d, J=8.8 Hz, 1H), 7.96 (s, 1H), 7.83 (d, J=8.0 Hz, 1H), 5.17-5.12 (m, 1H), 4.55 (d, J=17.4 Hz, 1H), 4.43 (d, J=17.4 Hz, 1H), 4.09 (s, 3H), 3.95 (s, 2H), 2.98-2.87 (m, 1H), 2.62 (d, J=18.0 Hz, 1H), 2.55 (s, 4H), 2.43 (dd, J=13.2, 4.4 Hz, 1H), 2.09-2.01 (m, 1H), 1.75 (s, 4H).

### Example 236:3-(5-(8-(isopropylamino)-4-(pyrrolidin-1-ylmethyl)-1,5-naphthyridin-2-yl)-1-oxoisoindolin-2-yl) piperidine-2,6-dione

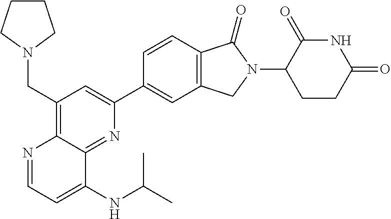 [SMILES: CC(C)Nc1ccnc2c(CN3CCCC3)cc(-c3ccc4c(c3)CN(C3CCC(=O)NC3=O)C4=O)nc12]

### Step A: 6-chloro-N-isopropyl-8-(pyrrolidin-1-ylmethyl)-1,5-naphthyridin-4-amine

To a solution of 6-chloro-8-(pyrrolidin-1-ylmethyl)-1,5-naphthyridin-4-yl trifluoro-methanesulfonate (Intermediate 91, 70.0 mg, 177 μmol, 1.0 eq) in DCM (2.00 mL) was added propan-2-amine (105 mg, 1.77 mmol, 10.0 eq) and the reaction mixture was stirred at 25° C. for 16 h. After evaporation, the crude product was purified by Prep-TLC(DCM/MeOH=10/1 v/v) to obtain 6-chloro-N-isopropyl-8-(pyrrolidin-1-ylmethyl)-1,5-naphthyridin-4-amine (20.0 mg, yield 37%) as a yellow oil. LC-MS (ESI): mass calced for: C₁₆H₂₁ClN₄, 304.82; m/z found, 305.2 [M+H]⁺.

### Step B: 3-(5-(8-(isopropylamino)-4-(pyrrolidin-1-ylmethyl)-1,5-naphthyridin-2-yl)-1-oxoisoindolin-2-yl) piperidine-2,6-dione

To a solution of 6-chloro-N-isopropyl-8-(pyrrolidin-1-ylmethyl)-1,5-naphthyridin-4-amine (20.0 mg, 65.6 μmol, 1 eq) in 1,4-Dioxane (2.00 mL) and Water (0.20 mL) were added 3-(1-oxo-5-(4,4,5,5-tetramethyl-1,3,2-dioxaborolan-2-yl) isoindolin-2-yl) piperidine-2,6-dione (Intermediate 13, 29.1 mg, 78.7 μmol, 1.2 eq), Potassium phosphate tribasic (41.8 mg, 197 μmol, 3.0 eq), and 1,1′-Bis(di-t-butylphosphino) ferrocene palladium dichloride (4.28 mg, 6.56 μmol, 0.1 eq). The mixture was stirred under N₂ at 95° C. for 1 h. After cooled to room temperature, the reaction mixture was filtered and the filtrate was concentrated in vacuum. The crude product was purified by Prep-HPLC with YMC-Actus Triart C18 (5 μm, 21.2×250 mm), and mobile phase of 5-95% ACN in water (0.1% HCOOH) over 23 min and then hold at 95% ACN for 3 min to give 3-(5-(8-(isopropylamino)-4-(pyrrolidin-1-ylmethyl)-1,5-naphthyridin-2-yl)-1-oxoisoindolin-2-yl) piperidine-2,6-dione (1.50 mg, yield 4%) as a yellow solid. LC-MS (ESI): mass calced for: C₂₉H₂₉N₅O₄, 511.58; m/z found, 512.2 [M+H]⁺. ¹H NMR (400 MHZ, DMSO-d₆) δ 11.03 (s, 1H), 8.51 (s, 1H), 8.49-8.42 (m, 2H), 8.31 (s, 1H), 7.89 (d, J=8.0 Hz, 1H), 7.02 (d, J=8.4 Hz, 1H), 6.72 (d, J=5.6 Hz, 1H), 5.20-5.15 (m, 1H), 4.61 (d, J=17.4 Hz, 1H), 4.48 (d, J=17.4 Hz, 1H), 4.27 (s, 2H), 3.93 (dd, J=14.6, 6.4 Hz, 1H), 3.00-2.87 (m, 1H), 2.64 (s, 5H), 2.47-2.42 (dd, J=13.2, 4.6 Hz, 1H), 2.11-2.00 (m, 1H), 1.79 (s, 4H), 1.35 (d, J=6.4 Hz, 6H).

### Example 237:3-(5-(8-(dimethylamino)-4-(pyrrolidin-1-ylmethyl)-1,5-naphthyridin-2-yl)-1-oxoisoindolin-2-yl) piperidine-2,6-dione

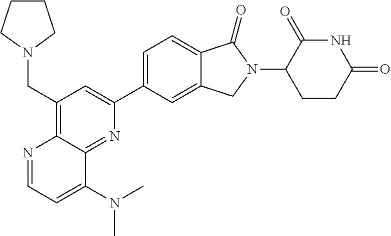 [SMILES: CN(C)c1ccnc2c(CN3CCCC3)cc(-c3ccc4c(c3)CN(C3CCC(=O)NC3=O)C4=O)nc12]

The title compound was prepared in a manner analogous to Example 236 by displacement of 6-chloro-8-(pyrrolidin-1-ylmethyl)-1,5-naphthyridin-4-yl trifluoromethanesulfonate (Intermediate 91) with dimethyl amine followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₈H₃₀N₆O₃, 498.1; m/z found, 499.1 [M+H]⁺. ¹H NMR (400 MHZ, DMSO-d₆) δ 11.02 (s, 1H), 8.49 (s, 1H), 8.42-8.30 (m, 3H), 7.91 (d, J=8.0 Hz, 1H), 6.85 (s, 1H), 5.19-5.14 (m, 1H), 4.60 (d, J=17.4 Hz, 1H), 4.53-4.26 (m, 3H), 3.46 (s, 6H), 2.93 (d, J=13.8 Hz, 1H), 2.64 (d, J=25.8 Hz, 5H), 2.43 (s, 1H), 2.06 (s, 1H), 1.84 (s, 4H).

### Example 238:3-(5-(3-isopropoxy-7-(pyrrolidin-1-ylmethyl)-1H-pyrazolo[4,3-b]pyridin-5-yl)-1-oxoisoindolin-2-yl) piperidine-2,6-dione

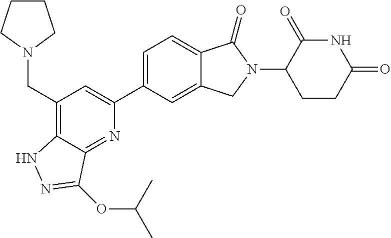 [SMILES: CC(C)Oc1n[nH]c2c(CN3CCCC3)cc(-c3ccc4c(c3)CN(C3CCC(=O)NC3=O)C4=O)nc12]

The title compound was prepared in a manner analogous to Example 1 by reductive amination between pyrrolidine and 5-chloro-3-isopropoxy-1-(tetrahydro-2H-pyran-2-yl)-1H-pyrazolo[4,3-b]pyridine-7-carbaldehyde (Intermediate 86) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) and de-protection of N-THP group as a white solid. LC-MS (ESI): mass calced for: C₂₇H₃₀N₆O₄, 502.2; m/z found, 503.2 [M+H]⁺. ¹H NMR (400 MHZ, DMSO-d₆) δ 12.62 (s, 1H), 11.02 (s, 1H), 10.25 (s, 1H), 8.35 (s, 1H), 8.29-8.24 (m, 2H), 7.90 (d, J=8.0 Hz, 1H), 5.23 (dd, J=12.2, 6.2 Hz, 1H), 5.16 (dd, J=13.2, 5.2 Hz, 1H), 4.71 (s, 2H), 4.58 (d, J=17.4 Hz, 1H), 4.46 (d, J=17.4 Hz, 1H), 3.56 (s, 2H), 3.26 (s, 2H), 2.99-2.88 (m, 1H), 2.63 (d, J=16.8 Hz, 1H), 2.45 (dd, J=13.2, 4.6 Hz, 1H), 2.15-2.01 (m, 3H), 1.91 (d, J=5.8 Hz, 2H), 1.45 (d, J=6.0 Hz, 6H).

### Example 239:3-(5-(8-cyclobutoxy-4-(pyrrolidin-1-ylmethyl)-1,5-naphthyridin-2-yl)-1-oxoisoindolin-2-yl) piperidine-2,6-dione

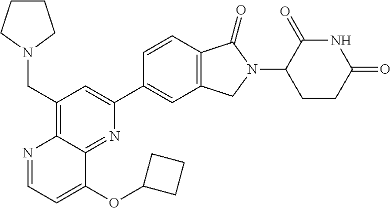 [SMILES: O=C1CCC(N2Cc3cc(-c4cc(CN5CCCC5)c5nccc(OC6CCC6)c5n4)ccc3C2=O)C(=O)N1]

The title compound was prepared in a manner analogous to Example 203 by alkylation between pyrrolidine and 4-(bromomethyl)-2-chloro-8-cyclobutoxy-1,5-naphthyridine (Intermediate 87) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₃₀H₃₁N₅O₄, 525.61; m/z found, 526.2 [M+H]⁺. ¹H NMR (400 MHz, DMSO-d₆) δ 11.03 (s, 1H), 8.79 (d, J=5.2 Hz, 1H), 8.51 (s, 1H), 8.48 (s, 1H), 8.42 (d, J=8.0 Hz, 1H), 8.17 (s, 1H), 7.95 (d, J=8.0 Hz, 1H), 7.20 (s, 1H), 5.21-5.16 (m, 1H), 5.12-5.03 (m, 1H), 4.63 (d, J=17.4 Hz, 1H), 4.57-4.46 (m, 3H), 3.01-2.81 (m, 5H), 2.64 (d, J=11.0 Hz, 3H), 2.48-2.39 (m, 1H), 2.35-2.23 (m, 2H), 2.11-2.03 (m, 1H), 1.96-1.82 (m, 5H), 1.80-1.72 (m, H).

### Example 240:3-(5-(3-methyl-1-(oxetan-3-yl)-4-(pyrrolidin-1-ylmethyl)-1H-pyrrolo[2,3-b]pyridin-6-yl)-1-oxoisoindolin-2-yl) piperidine-2,6-dione

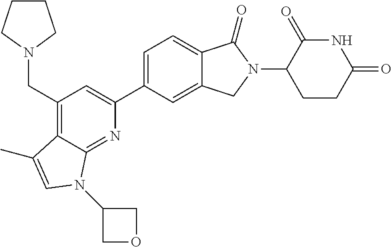 [SMILES: Cc1cn(C2COC2)c2nc(-c3ccc4c(c3)CN(C3CCC(=O)NC3=O)C4=O)cc(CN3CCCC3)c12]

The title compound was prepared in a manner analogous to Example 1 by reductive amination between pyrrolidine and 6-chloro-3-methyl-1-(oxetan-3-yl)-1H-pyrrolo[2,3-b]pyridine-4-carbaldehyde (Intermediate 88) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₉H₃₁N₅O₄, 513.2; m/z found, 514.3 [M+H]⁺. ¹H NMR (400 MHZ, DMSO-d₆) δ 11.01 (s, 1H), 8.37 (s, 1H), 8.32 (d, J=8.0 Hz, 1H), 8.22 (s, 1H), 7.83 (d, J=8.0 Hz, 1H), 7.73 (d, J=10.8 Hz, 2H), 6.13-6.05 (m, 1H), 5.15 (dd, J=13.2, 5.2 Hz, 1H), 5.05 (t, J=7.2 Hz, 2H), 5.00 (t, J=6.8 Hz, 2H), 4.56 (d, J=17.2 Hz, 1H), 4.43 (d, J=17.2 Hz, 1H), 3.99 (s, 2H), 2.92 (dd, J=13.2, 5.2 Hz, 1H), 2.67-2.58 (m, 1H), 2.54 (s, 7H), 2.46-2.39 (m, 1H), 2.07-1.99 (m, 1H), 1.72 (s, 4H).

### Example 241:3-(5-(8-cyclobutyl-4-(pyrrolidin-1-ylmethyl) quinolin-2-yl)-1-oxoisoindolin-2-yl) piperidine-2,6-dione

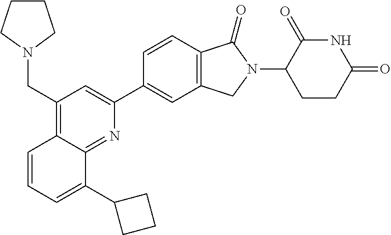 [SMILES: O=C1CCC(N2Cc3cc(-c4cc(CN5CCCC5)c5cccc(C6CCC6)c5n4)ccc3C2=O)C(=O)N1]

The title compound was prepared in a manner analogous to Example 1 by Suzuki coupling of 8-cyclobutyl-4-(pyrrolidin-1-ylmethyl) quinolin-2-yl trifluoromethanesulfonate (Intermediate 89) with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₃₁H₃₂N₄O₃, 508.62; m/z found, 509.3 [M+H]⁺. ¹H NMR (400 MHZ, DMSO-d₆) δ 11.00 (s, 1H), 8.50 (s, 1H), 8.46 (d, J=8.0 Hz, 1H), 8.18 (s, 1H), 8.17 (s, 1H), 8.15 (d, J=8.2 Hz, 1H), 7.92 (d, J=8.0 Hz, 1H), 7.68 (d, J=7.0 Hz, 1H), 7.59 (t, J=7.8 Hz, 1H), 5.20-5.15 (m, 1H), 4.67-4.56 (m, 2H), 4.49 (d, J=17.2 Hz, 1H), 4.12 (s, 2H), 3.00-2.90 (m, 1H), 2.69-2.53 (m, 7H), 2.48-2.39 (m, 1H), 2.33-2.23 (m, 2H), 2.00-2.15 (m, 1H), 2.10-2.02 (m, 1H), 1.94-1.89 (m, 1H), 1.74 (s, 4H).

### Example 242:3-(5-(8-cyclopropoxy-4-(pyrrolidin-1-ylmethyl)-1,5-naphthyridin-2-yl)-1-oxoisoindolin-2-yl) piperidine-2,6-dione

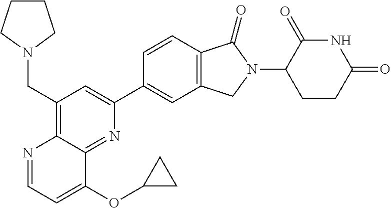 [SMILES: O=C1CCC(N2Cc3cc(-c4cc(CN5CCCC5)c5nccc(OC6CC6)c5n4)ccc3C2=O)C(=O)N1]

The title compound was prepared in a manner analogous to Example 203 by alkylation between pyrrolidine and 4-(bromomethyl)-2-chloro-8-cyclopropoxy-1,5-naphthyridine (Intermediate 90) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₉H₂₉N₅O₄, 511.58; m/z found, 512.2 [M+H]⁺. ¹H NMR (400 MHz, DMSO-d₆) δ 11.03 (s, 1H), 8.89 (d, J=5.2 Hz, 1H), 8.52 (s, 1H), 8.45 (s, 1H), 8.39 (d, J=8.0 Hz, 1H), 8.16 (s, 1H), 7.94 (d, J=8.0 Hz, 1H), 7.64 (d, J=4.6 Hz, 1H), 5.20-5.15 (m, 1H), 4.61 (t, J=11.0 Hz, 3H), 4.49 (d, J=17.4 Hz, 1H), 4.22 (s, 1H), 3.00-2.88 (m, 5H), 2.64 (d, J=17.0 Hz, 1H), 2.48-2.39 (m, 1H), 2.10-2.02 (m, 1H), 1.88 (s, 4H), 1.00 (t, J=5.6 Hz, 2H), 0.92 (s, 2H).

### Example 243:3-(5-(8-(azetidin-1-yl)-4-(pyrrolidin-1-ylmethyl)-1,5-naphthyridin-2-yl)-1-oxoisoindolin-2-yl) piperidine-2,6-dione

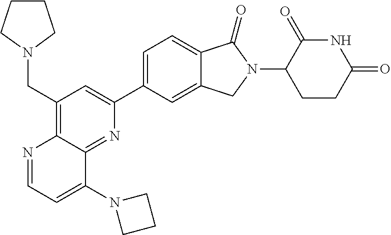 [SMILES: O=C1CCC(N2Cc3cc(-c4cc(CN5CCCC5)c5nccc(N6CCC6)c5n4)ccc3C2=O)C(=O)N1]

The title compound was prepared in a manner analogous to Example 236 by displacement of 6-chloro-8-(pyrrolidin-1-ylmethyl)-1,5-naphthyridin-4-yl trifluoromethanesulfonate (Intermediate 91) with azetidine followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₉H₃₀N₆O₃, 510.24; m/z found, 511.6 [M+H]⁺. ¹H NMR (400 MHz, DMSO-d₆) δ 11.02 (s, 1H), 8.39 (d, J=5.2 Hz, 1H), 8.31 (dd, J=17.6, 11.2 Hz, 3H), 8.15 (s, 1H), 7.89 (d, J=8.0 Hz, 1H), 6.40 (d, J=5.23 Hz, 1H), 5.19-5.14 (m, 1H), 4.59 (d, J=17.4 Hz, 1H), 4.46 (d, J=17.4 Hz, 1H), 4.35 (s, 2H), 4.34-3.91 (m, 4H), 2.99-2.90 (m, 1H), 2.76 (s, 4H), 2.63 (d, J=17.2 Hz, 1H), 2.47-2.41 (m, 3H), 2.10-2.01 (m, 1H), 1.82 (s, 4H).

### Example 244:3-(5-(8-(cyclopropylamino)-4-(pyrrolidin-1-ylmethyl)-1,5-naphthyridin-2-yl)-1-oxoisoindolin-2-yl) piperidine-2,6-dione

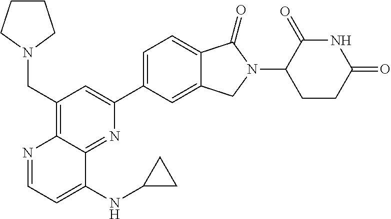 [SMILES: O=C1CCC(N2Cc3cc(-c4cc(CN5CCCC5)c5nccc(NC6CC6)c5n4)ccc3C2=O)C(=O)N1]

The title compound was prepared in a manner analogous to Example 236 by displacement of 6-chloro-8-(pyrrolidin-1-ylmethyl)-1,5-naphthyridin-4-yl trifluoromethanesulfonate (Intermediate 91) with cyclopropanamine followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₉H₃₀N₆O₃, 510.2; m/z found, 511.2 [M+H]⁺. ¹H NMR (400 MHZ, DMSO-d₆) δ 11.03 (s, 1H), 8.60 (s, 1H), 8.53 (dd, J=14.6, 6.8 Hz, 2H), 8.34 (s, 1H), 8.22 (s, 1H), 7.86 (d, J=8.0 Hz, 1H), 7.72 (s, 1H), 6.96 (d, J=5.2 Hz, 1H), 5.20-5.15 (m, 1H), 4.59 (d, J=17.4 Hz, 1H), 4.47 (d, J=17.4 Hz, 1H), 4.29 (s, 2H), 2.96-2.89 (m, 1H), 2.65 (s, 6H), 2.47-2.39 (m, 1H), 2.10-2.02 (m, 1H), 1.79 (s, 4H), 0.93-0.86 (m, 2H), 0.71 (d, J=2.8 Hz, 2H).

### Example 245:3-(5-(1-(oxetan-3-yl)-4-(pyrrolidin-1-ylmethyl)-3-(trifluoromethyl)-1H-pyrazolo[3,4-b]pyridin-6-yl)-1-oxoisoindolin-2-yl) piperidine-2,6-dione

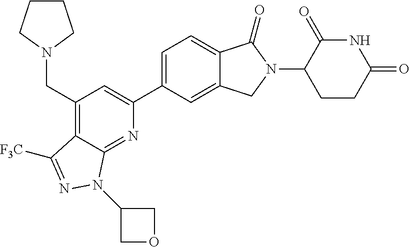 [SMILES: O=C1CCC(N2Cc3cc(-c4cc(CN5CCCC5)c5c(C(F)(F)F)nn(C6COC6)c5n4)ccc3C2=O)C(=O)N1]

The title compound was prepared in a manner analogous to Example 1 by reductive amination between pyrrolidine and 6-chloro-1-(oxetan-3-yl)-3-(trifluoromethyl)-1H-pyrazolo[3,4-b]pyridine-4-carbaldehyde (Intermediate 92) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₈H₂₇F₃N₆O₄, 568.2; m/z found, 569.1 [M+H]⁺. ¹H NMR (400 MHZ, DMSO-d₆) δ 11.03 (s, 1H), 8.50 (s, 2H), 8.14 (s, 1H), 7.93 (d, J=8.0 Hz, 1H), 6.42 (s, 1H), 5.20-5.15 (m, 1H), 5.12 (d, J=7.0 Hz, 4H), 4.61 (d, J=17.6 Hz, 1H), 4.48 (d, J=17.6 Hz, 1H), 4.03 (s, 2H), 2.97-2.89 (m, 1H), 2.63 (d, J=17.6 Hz, 5H), 2.43 (d, J=12.8 Hz, 1H), 2.08-2.04 (m, 1H), 1.76 (s, 4H).¹⁹F NMR (400 MHZ, DMSO-d₆) δ-73.43 (ppm).

### Example 246:3-(5-(3-methyl-1-(oxetan-3-yl)-4-(pyrrolidin-1-ylmethyl)-1H-pyrazolo[3,4-b]pyridin-6-yl)-1-oxoisoindolin-2-yl) piperidine-2,6-dione

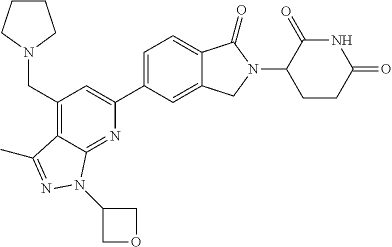 [SMILES: Cc1nn(C2COC2)c2nc(-c3ccc4c(c3)CN(C3CCC(=O)NC3=O)C4=O)cc(CN3CCCC3)c12]

The title compound was prepared in a manner analogous to Example 1 by reductive amination between pyrrolidine and 6-chloro-3-methyl-1-(oxetan-3-yl)-1H-pyrazolo[3,4-b]pyridine-4-carbaldehyde (Intermediate 93) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₈H₃₀N₆O₄, 514.23; m/z found, 515.23 [M+H]⁺. ¹H NMR (400 MHZ, DMSO-d₆) δ 11.04 (s, 1H), 8.44 (s, 1H), 8.38 (d, J=8.2 Hz, 1H), 7.88 (d, J=8.2 Hz, 1H), 7.82 (s, 1H), 6.31-6.17 (m, 1H), 5.19-5.14 (m, 1H), 5.10 (t, J=6.4 Hz, 2H), 5.04 (t, J=7.0 Hz, 2H), 4.58 (d, J=17.4 Hz, 1H), 4.45 (d, J=17.4 Hz, 1H), 4.03 (s, 2H), 3.01-2.87 (m, 1H), 2.73 (s, 3H), 2.63 (d, J=16.2 Hz, 1H), 2.55 (s, 4H), 2.46 (d, J=13.2 Hz, 1H), 2.10-2.00 (m, 1H), 1.73 (s, 4H).

### Example 247:3-(5-(8-cyclobutyl-4-(pyrrolidin-1-ylmethyl)-1,5-naphthyridin-2-yl)-1-oxoisoindolin-2-yl) piperidine-2,6-dione

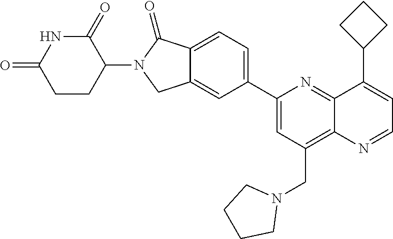 [SMILES: O=C1CCC(N2Cc3cc(-c4cc(CN5CCCC5)c5nccc(C6CCC6)c5n4)ccc3C2=O)C(=O)N1]

### Step A: 2-chloro-8-cyclobutyl-4-(pyrrolidin-1-ylmethyl)-1,5-naphthyridine

ZnCl2 (0.5 M in THF) (30 mL, 15 mmol, 1.0 eq) was added dropwise to a solution of cyclobutylmagnesium bromide (0.5 M in THF) (30 mL, 15 mmol, 1.0 eq) in dry THF (20 mL) under N₂ at 0° C. and the mixture was stirred at 0° C. for 30 min. The solution was directly used in next step without further purification.

To a solution of 6-chloro-8-(pyrrolidin-1-ylmethyl)-1,5-naphthyridin-4-yl trifluoromethanesulfonate (Intermediate 91, 200 mg, 505 μmol, 1.0 eq), 1,1′— Bis(diphenylphosphino) ferrocene-palladium (II) dichloride (74.0 mg, 101 μmol, 0.2 eq), and Lithium chloride (64.3 mg, 1.52 mmol, 3.0 eq) in THF (10.0 mL) and NMP (2.0 mL) was added dropwise cyclobutylzinc (II) chloride (630 mg, 4.04 mmol, 8.0 eq) under N₂ at 0° C. The reaction was stirred at room temperature for 2 h. The the reaction mixture was quenched with saturated aqueous NH₄Cl solution (20 mL) and extracted with EA (30 mL×4). The organic layer was washed with brine (30 mL), dried over anhydrous sodium sulfate, filtered and concentrated under reduced pressure. The residue was purified with Prep-TLC(DCM/MeOH=15/1 v/v) to give 2-chloro-8-cyclobutyl-4-(pyrrolidin-1-ylmethyl)-1,5-naphthyridine (25.0 mg, yield 16%) as a brown solid. LC-MS (ESI): mass calcd. For C₁₇H₂OClN₃, 301.13; m/z found, 302.1 [M+H]⁺.

### Step B: 3-(5-(8-cyclobutyl-4-(pyrrolidin-1-ylmethyl)-1,5-naphthyridin-2-yl)-1-oxoisoindolin-2-yl) piperidine-2,6-dione

To a solution of 3-(1-oxo-5-(4,4,5,5-tetramethyl-1,3,2-dioxaborolan-2-yl) isoindolin-2-yl) piperidine-2,6-dione (Intermediate 13, 29.4 mg, 79.5 μmol, 1.2 eq), 2-chloro-8-cyclobutyl-4-(pyrrolidin-1-ylmethyl)-1,5-naphthyridine (20.0 mg, 66.3 μmol, 1.0 eq), and K₃PO₄ (42.2 mg, 199 μmol, 3.0 eq) in 1,4-Dioxane (2.0 mL) and H₂O (0.2 mL) was added Pd (dtbpf) Cl₂ (8.6 mg, 13.3 μmol, 0.2 eq) at room temperature. The reaction mixture was stirred under N₂ at 90° C. for 1 h. After cooled to room temperature, the reaction mixture was quenched with water (5 mL) and extracted with EtOAc (5 mL×3). The organic layer was washed with brine (5 mL), dried over Na₂SO₄, filtered and concentrated under reduced pressure. The residue was purified by Prep-TLC(DCM/MeOH=10/1 v/v) to afford 3-(5-(8-cyclobutyl-4-(pyrrolidin-1-ylmethyl)-1,5-naphthyridin-2-yl)-1-oxoisoindolin-2-yl) piperidine-2,6-dione formate (5.5 mg, yield 16%) as a brown solid. LC-MS (ESI): mass calcd. For C₃₀H₃₁N₅O₃, 509.2; m/z found, 510.2 [M+H]⁺. ¹H NMR (400 MHZ, DMSO-d₆) δ 11.03 (s, 1H), 8.93 (d, J=4.4 Hz, 1H), 8.48 (s, 1H), 8.42 (s, 1H), 8.40 (s, 1H), 8.24 (s, 1H), 7.94 (d, J=8.0 Hz, 1H), 7.71 (d, J=4.4 Hz, 1H), 5.19-5.15 (m, 1H), 4.66-4.60 (m, 2H), 4.50 (d, J=17.4 Hz, 1H), 4.38 (s, 2H), 2.94 (d, J=13.2 Hz, 1H), 2.69-2.65 (m, 4H), 2.63-2.54 (m, 3H), 2.45 (d, J=13.6 Hz, 1H), 2.35-2.28 (m, 2H), 2.20 (dd, J=18.6, 9.2 Hz, 1H), 2.09-2.02 (m, 1H), 1.95-1.90 (m, 1H), 1.81-1.76 (m, 4H).

### Example 248:3-(5-(1-(1-methyl-1H-pyrazol-4-yl)-4-(pyrrolidin-1-ylmethyl)-1H-pyrrolo[2,3-b]pyridin-6-yl)-1-oxoisoindolin-2-yl) piperidine-2,6-dione

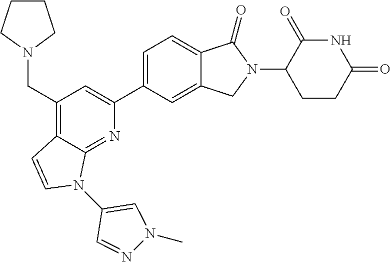 [SMILES: Cn1cc(-n2ccc3c(CN4CCCC4)cc(-c4ccc5c(c4)CN(C4CCC(=O)NC4=O)C5=O)nc32)cn1]

The title compound was prepared in a manner analogous to Example 1 by reductive amination between pyrrolidine and 6-chloro-1-(1-methyl-1H-pyrazol-4-yl)-1H-pyrrolo[2,3-b]pyridine-4-carbaldehyde (Intermediate 94) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₉H₂₉N₇O₃, 523.23; m/z found, 524.2 [M+H]⁺. ¹H NMR (400 MHZ, DMSO-d₆) δ 11.02 (s, 1H), 8.50 (s, 1H), 8.40 (s, 1H), 8.33 (d, J=8.0 Hz, 1H), 8.24 (s, 1H), 8.15 (s, 1H), 7.91 (d, J=3.6 Hz, 1H), 7.88-7.83 (m, 2H), 6.85 (d, J=3.6 Hz, 1H), 5.19-5.14 (m, 1H), 4.59 (d, J=17.4 Hz, 1H), 4.46 (d, J=17.4 Hz, 1H), 3.99 (s, 2H), 3.97 (s, 3H), 2.99-2.89 (m, 1H), 2.64 (t, J=12.8 Hz, 1H), 2.56 (s, 4H), 2.48-2.36 (m, 1H), 2.08-2.02 (m, 1H), 1.75 (s, 4H).

### Example 249:3-(5-(8-(difluoromethoxy)-4-(pyrrolidin-1-ylmethyl)-1,5-naphthyridin-2-yl)-1-oxoisoindolin-2-yl) piperidine-2,6-dione

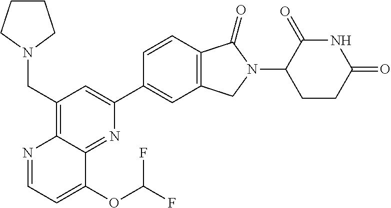 [SMILES: O=C1CCC(N2Cc3cc(-c4cc(CN5CCCC5)c5nccc(OC(F)F)c5n4)ccc3C2=O)C(=O)N1]

The title compound was prepared in a manner analogous to Example 203 by alkylation between pyrrolidine and 4-(bromomethyl)-2-chloro-8-(difluoromethoxy)-1,5-naphthyridine (Intermediate 95) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₇H₂₅F₂N₅O₄, 521.52; m/z found, 522.2 [M+H]⁺. ¹H NMR (400 MHZ, DMSO-d₆) δ 11.03 (s, 1H), 9.01 (d, J=5.0 Hz, 1H), 8.69 (s, 1H), 8.51 (s, 1H), 8.45 (d, J=8.0 Hz, 1H), 8.14 (s, 1H), 7.97 (d, J=8.0 Hz, 1H), 7.85 (t, J=72.8 Hz, 1H), 7.64 (d, J=5.0 Hz, 1H), 5.21-5.16 (m, 1H), 4.70 (s, 2H), 4.64 (d, J=17.6 Hz, 1H), 4.50 (d, J=17.6 Hz, 1H), 3.11-2.87 (m, 5H), 2.64 (d, J=16.8 Hz, 1H), 2.49-2.39 (m, 1H), 2.07 (dt, J=10.2, 5.0 Hz, 1H), 1.89 (s, 4H). ¹⁹F NMR (400 MHZ, DMSO-d₆) δ-83.57 (ppm).

### Example 250:3-(5-(4-(((S)-2-(methoxymethyl) pyrrolidin-1-yl)methyl)-1-(oxetan-3-yl)-1H-pyrrolo[2,3-b]pyridin-6-yl)-1-oxoisoindolin-2-yl) piperidine-2,6-dione

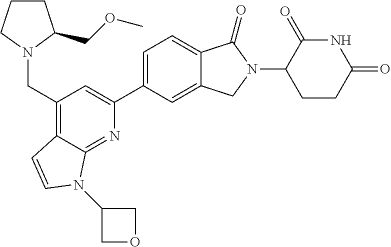 [SMILES: COC[C@@H]1CCCN1Cc1cc(-c2ccc3c(c2)CN(C2CCC(=O)NC2=O)C3=O)nc2c1ccn2C1COC1]

The title compound was prepared in a manner analogous to Example 1 by reductive amination between(s)-2-methoxymethyl-pyrrolidine and 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid (TFA salt). LC-MS (ESI): mass calced for: C₃₀H₃₃N₅O₅, 543.25; m/z found, 544.3 [M+H]⁺. ¹H NMR (400 MHZ, DMSO-d₆) δ 11.02 (s, 1H), 10.06 (s, 1H), 8.43 (s, 1H), 8.37 (d, J=8.0 Hz, 1H), 8.19 (s, 1H), 8.12 (s, 1H), 7.89 (d, J=7.6 Hz, 1H), 6.92 (s, 1H), 6.18-6.14 (m, 1H), 5.18-5.14 (m, 1H), 5.13-5.03 (m, 4H), 4.92-4.89 (m, 1H), 4.69-4.60 (m, 1H), 4.59 (d, J=17.2 Hz, 1H), 4.46 (d, J=17.2 Hz, 1H), 3.90 (s, 1H), 3.68 (d, J=16.8 Hz, 2H), 3.40 (s, 5H), 3.01-2.87 (m, 1H), 2.63 (d, J=17.0 Hz, 1H), 2.48-2.43 (m, 1H), 2.22 (s, 1H), 2.12-1.96 (m, 2H), 1.87 (s, 1H), 1.74 (s, 1H). ¹⁹F NMR (400 MHZ, DMSO-d6) δ -73.55 (ppm).

### Example 251:3-(5-(8-(oxetan-3-yloxy)-4-(pyrrolidin-1-ylmethyl)-1,5-naphthyridin-2-yl)-1-oxoisoindolin-2-yl) piperidine-2,6-dione

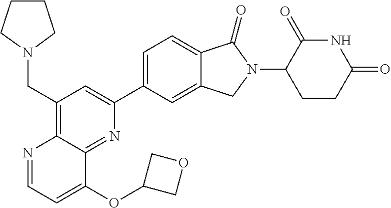 [SMILES: O=C1CCC(N2Cc3cc(-c4cc(CN5CCCC5)c5nccc(OC6COC6)c5n4)ccc3C2=O)C(=O)N1]

The title compound was prepared in a manner analogous to Example 203 by alkylation between pyrrolidine and 4-(bromomethyl)-2-chloro-8-(oxetan-3-yloxy)-1,5-naphthyridine (Intermediate 96) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₉H₂₉N₅O₅, 527.22; m/z found, 528.2 [M+H]⁺. ¹H NMR (400 MHz, DMSO-d₆) δ 11.03 (s, 1H), 8.80 (d, J=5.2 Hz, 1H), 8.53 (d, J=9.0 Hz, 1H), 8.51 (s, 1H), 8.44 (d, J=8.0 Hz, 1H), 8.14 (s, 1H), 7.95 (d, J=8.0 Hz, 1H), 7.00 (d, J=5.2 Hz, 1H), 5.70-5.62 (m, 1H), 5.20-5.14 (m, 1H), 5.117 (t, J=6.8 Hz, 2H), 4.80-4.75 (m, 2H), 4.63 (d, J=17.4 Hz, 1H), 4.55 (d, J=18.4 Hz, 2H), 4.50 (d, J=17.4 Hz, 1H), 2.99-2.90 (m, 5H), 2.67-2.62 (m, 1H), 2.47-2.43 (m, 1H), 2.11-2.03 (m, 1H), 1.87 (s, 4H).

### Example 252:3-(5-(4-((2-(difluoromethyl) pyrrolidin-1-yl)methyl)-1-(oxetan-3-yl)-1H-pyrrolo[2,3-b]pyridin-6-yl)-1-oxoisoindolin-2-yl) piperidine-2,6-dione

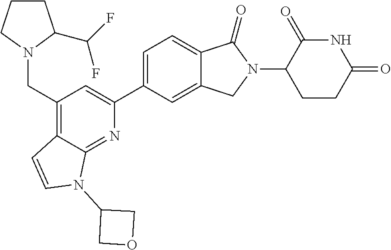 [SMILES: O=C1CCC(N2Cc3cc(-c4cc(CN5CCCC5C(F)F)c5ccn(C6COC6)c5n4)ccc3C2=O)C(=O)N1]

The title compound was prepared in a manner analogous to Example 1 by reductive amination between 2-(difluoromethyl) pyrrolidine and To a solution of 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₉H₂₉F₂N₅O₄, 549.22; m/z found, 550.3 [M+H]⁺. ¹H NMR (400 MHZ, DMSO-d₆) δ 11.01 (s, 1H), 8.38 (s, 1H), 8.33 (d, J=7.8 Hz, 1H), 8.00 (d, J=3.6 Hz, 1H), 7.84 (d, J=8.0 Hz, 1H), 7.79 (s, 1H), 6.79 (d, J=3.6 Hz, 1H), 6.15-6.01 (m, 2H), 5.18-5.13 (m, 1H), 5.08 (t, J=7.2 Hz, 4H), 4.57 (d, J=17.2 Hz, 1H), 4.46-4.33 (m, 2H), 3.93 (d, J=13.8 Hz, 1H), 3.18-3.10 (m, 1H), 3.00-2.90 (m, 1H), 2.83 (s, 1H), 2.66 (d, J=9.2 Hz, 1H), 2.46-2.38 (m, 2H), 2.04 (d, J=7.1 Hz, 1H), 2.00-1.90 (m, 1H), 1.82-1.76 (m, 1H), 1.75-1.58 (m, 2H). ¹⁹F NMR (400 MHZ, DMSO-d6) δ-125.30-124.46 (ppm).

### Example 253:3-(5-(1-(oxetan-3-yl)-4-((2-(trifluoromethyl) pyrrolidin-1-yl)methyl)-1H-pyrrolo[2,3-b]pyridin-6-yl)-1-oxoisoindolin-2-yl) piperidine-2,6-dione

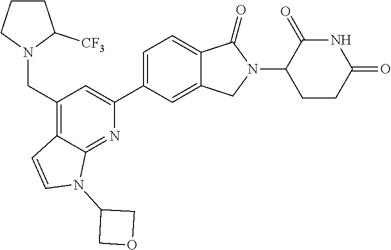 [SMILES: O=C1CCC(N2Cc3cc(-c4cc(CN5CCCC5C(F)(F)F)c5ccn(C6COC6)c5n4)ccc3C2=O)C(=O)N1]

The title compound was prepared in a manner analogous to Example 1 by reductive amination between 2-(trifluoromethyl) pyrrolidine and To a solution of 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₉H₂₈F₃N₅O₄, 567.21; m/z found, 568.0 [M+H]⁺. ¹H NMR (400 MHZ, DMSO-d₆) δ 11.01 (s, 1H), 8.37 (s, 1H), 8.32 (d, J=8.0 Hz, 1H), 8.01 (d, J=3.4 Hz, 1H), 7.85 (d, J=8.0 Hz, 1H), 7.77 (s, 1H), 6.75 (d, J=3.4 Hz, 1H), 6.17-6.09 (m, 1H), 5.15-5.10 (m, 1H), 5.08-5.06 (m, 4H), 4.58 (d, J=17.2 Hz, 1H), 4.40-4.05 (m, 2H), 4.04 (d, J=13.8 Hz, 1H), 3.65 (t, J=8.2 Hz, 1H), 3.00-2.88 (m, 1H), 2.84 (t, J=8.0 Hz, 1H), 2.63 (d, J=16.4 Hz, 1H), 2.47-2.27 (m, 2H), 2.17-1.99 (m, 2H), 1.95-1.84 (m, 1H), 1.80-1.72 (m, 2H). ¹⁹F NMR (400 MHZ, DMSO-d₆) δ-74.59 (ppm).

### Example 254:3-(5-(4-((2,2-dimethylpyrrolidin-1-yl)methyl)-1-(oxetan-3-yl)-1H-pyrrolo[2,3-b]pyridin-6-yl)-1-oxoisoindolin-2-yl) piperidine-2,6-dione

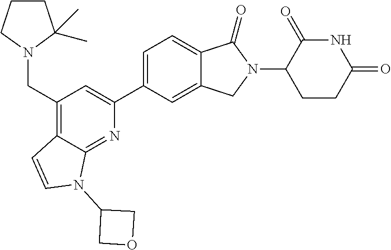 [SMILES: CC1(C)CCCN1Cc1cc(-c2ccc3c(c2)CN(C2CCC(=O)NC2=O)C3=O)nc2c1ccn2C1COC1]

The title compound was prepared in a manner analogous to Example 1 by reductive amination between 2,2-dimethyl-pyrrolidine and To a solution of 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₃₀H₃₃N₅O₄, 527.25; m/z found, 528.2 [M+H]⁺. ¹H NMR (400 MHZ, DMSO-d₆) δ 11.01 (s, 1H), 8.37 (s, 1H), 8.32 (d, J=7.6 Hz, 2H), 7.96 (d, J=3.6 Hz, 1H), 7.84 (d, J=7.8 Hz, 1H), 7.77 (s, 1H), 6.78 (d, J=3.6 Hz, 1H), 6.16-6.08 (m, 1H), 5.18-5.13 (m, 1H), 5.07 (d, J=7.6 Hz, 4H), 4.57 (d, J=17.4 Hz, 1H), 4.44 (d, J=17.4 Hz, 1H), 3.87 (s, 2H), 2.98-2.90 (m, 1H), 2.67-2.58 (m, 3H), 2.46-2.42 (m, 1H), 2.09-2.02 (m, 1H), 1.67 (s, 4H), 1.16 (s, 6H).

### Example 255:3-(5-(1-(3-methoxycyclobutyl)-4-(pyrrolidin-1-ylmethyl)-1H-pyrrolo[2,3-b]pyridin-6-yl)-1-oxoisoindolin-2-yl) piperidine-2,6-dione

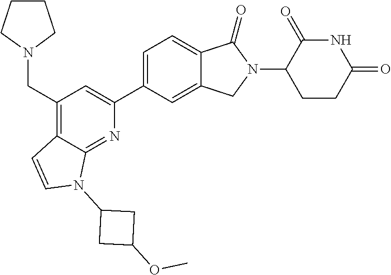 [SMILES: COC1CC(n2ccc3c(CN4CCCC4)cc(-c4ccc5c(c4)CN(C4CCC(=O)NC4=O)C5=O)nc32)C1]

The title compound was prepared in a manner analogous to Example 1 by Suzuki coupling of 6-chloro-1-(3-methoxycyclobutyl)-4-(pyrrolidin-1-ylmethyl)-1H-pyrrolo[2,3-b]pyridine (Intermediate 97) with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₃₀H₃₃N₅O₄, 527.25; m/z found, 528.2 [M+H]⁺. ¹H NMR (400 MHZ, DMSO-d6) δ 11.02 (s, 1H), 8.35 (s, 1H), 8.30 (d, J=9.2 Hz, 2H), 7.84 (dd, J=10.8, 6.0 Hz, 2H), 7.76 (s, 1H), 6.69 (d, J=3.6 Hz, 1H), 5.66-5.50 (m, 1H), 5.17-5.12 (m, 1H), 4.59 (d, J=17.4 Hz, 1H), 4.45 (d, J=17.4 Hz, 1H), 4.22 (s, 1H), 3.95 (s, 2H), 3.27 (s, 3H), 2.92 (d, J=12.6 Hz, 1H), 2.76 (d, J=6.6 Hz, 2H), 2.64-2.53 (m, 7H), 2.44 (d, J=12.6 Hz, 1H), 2.06 (d, J=6.45 Hz, 1H), 1.74 (s, 4H).

### Example 256:3-(5-(1-(1-acetylazetidin-3-yl)-4-(pyrrolidin-1-ylmethyl)-1H-pyrrolo[2,3-b]pyridin-6-yl)-1-oxoisoindolin-2-yl) piperidine-2,6-dione

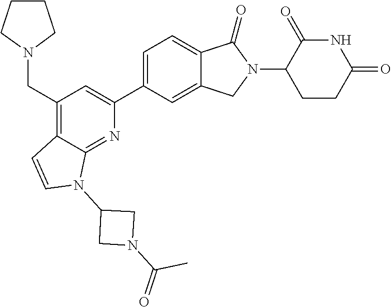 [SMILES: CC(=O)N1CC(n2ccc3c(CN4CCCC4)cc(-c4ccc5c(c4)CN(C4CCC(=O)NC4=O)C5=O)nc32)C1]

The title compound was prepared in a manner analogous to Example 1 by Suzuki coupling of 1-(3-(6-chloro-4-(pyrrolidin-1-ylmethyl)-1H-pyrrolo[2,3-b]pyridin-1-yl) azetidin-1-yl) ethan-1-one (Intermediate 98) with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₃₀H₃₂N₆O₄, 540.3; m/z found, 541.3 [M+H]⁺. ¹H NMR (400 MHZ, DMSO-d₆) δ 11.01 (s, 1H), 8.37 (s, 1H), 8.30 (d, J=8.0 Hz, 1H), 8.19 (s, 1H), 7.83 (dd, J=9.6, 5.4 Hz, 3H), 6.74 (d, J=3.4 Hz, 1H), 5.76-5.64 (m, 1H), 5.18-5.13 (m, 1H), 4.70-4.64 (m, 2H), 4.55 (dd, J=17.2, 3.2 Hz, 1H), 4.45-4.34 (m, 3H), 3.98 (s, 2H), 2.96-2.89 (m, 1H), 2.67-2.54 (m, 5H), 2.47-2.44 (m, 1H), 2.08-2.01 (m, 1H), 1.88 (d, J=4.4 Hz, 3H), 1.77-1.71 (m, 4H).

### Example 257:3-(5-(4-((2-(fluoromethyl) pyrrolidin-1-yl)methyl)-1-(oxetan-3-yl)-1H-pyrrolo[2,3-b]pyridin-6-yl)-1-oxoisoindolin-2-yl) piperidine-2,6-dione

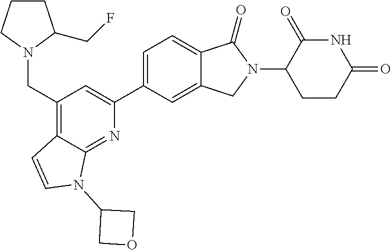 [SMILES: O=C1CCC(N2Cc3cc(-c4cc(CN5CCCC5CF)c5ccn(C6COC6)c5n4)ccc3C2=O)C(=O)N1]

The title compound was prepared in a manner analogous to Example 1 by Suzuki coupling of 16-chloro-4-((2-(fluoromethyl) pyrrolidin-1-yl)methyl)-1-(oxetan-3-yl)-1H-pyrrolo[2,3-b]pyridine (Intermediate 99) with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₉H₃₀FN504, 531.1; m/z found, 532.1 [M+H]⁺. ¹H NMR (400 MHZ, DMSO-d₆) δ 11.01 (s, 1H), 8.38 (s, 1H), 8.33 (d, J=8.0 Hz, 1H), 8.17 (s, 1H), 7.99 (s, 1H), 7.84 (d, J=8.0 Hz, 1H), 7.78 (s, 1H), 6.80 (dd, J=11.2, 3.4 Hz, 1H), 6.17-6.08 (m, 1H), 5.18-5.13 (m, 1H), 5.07 (d, J=7.2 Hz, 4H), 4.79-4.50 (m, 2H), 4.41 (dd, J=25.0, 15.4 Hz, 2H), 3.90 (s, 1H), 3.81 (d, J=13.6 Hz, 1H), 2.92 (d, J=12.8 Hz, 1H), 2.79 (dd, J=25.4, 13.6 Hz, 1H), 2.63 (d, J=17.8 Hz, 1H), 2.45 (d, J=13.6 Hz, 1H), 2.34 (d, J=8.2 Hz, 1H), 2.10-2.00 (m, 1H), 1.85-1.41 (m, 4H), 1.16 (dd, J=12.2, 7.2 Hz, 1H). 1° F. NMR (400 MHZ, DMSO-d₆) δ-220.85 (ppm).

### Example 258:3-(5-(2-methyl-3-(oxetan-3-yl)-7-(pyrrolidin-1-ylmethyl)-3H-imidazo[4,5-b]pyridin-5-yl)-1-oxoisoindolin-2-yl) piperidine-2,6-dione

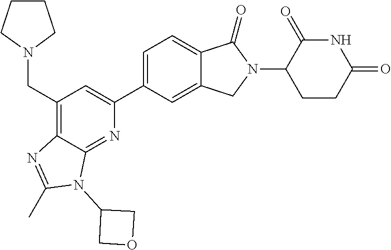 [SMILES: Cc1nc2c(CN3CCCC3)cc(-c3ccc4c(c3)CN(C3CCC(=O)NC3=O)C4=O)nc2n1C1COC1]

The title compound was prepared in a manner analogous to Example 203 by alkylation between pyrrolidine and 7-(bromomethyl)-5-chloro-2-methyl-3-(oxetan-3-yl)-3H-imidazo[4,5-b]pyridine (Intermediate 100a) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calcd. for C₂₈H₃₀N₆O₄, 514.23; m/z found, 515.34 [M+H]⁺. ¹H NMR (400 MHZ, DMSO-d₆) δ 11.01 (s, 1H), 8.35 (s, 1H), 8.31 (d, J=8.2 Hz, 1H), 8.18 (s, 1H), 7.92 (s, 1H), 7.85 (d, J=7.9 Hz, 1H), 5.88-5.79 (m, 1H), 5.55 (s, 2H), 5.21-5.12 (m, 1H), 5.00 (t, J=7.0 Hz, 2H), 4.58 (d, J=17.2 Hz, 1H), 4.44 (d, J=17.2 Hz, 1H), 4.03 (s, 2H), 2.99-2.90 (m, 1H), 2.66 (d, J=10.0 Hz, 1H), 2.61 (s, 3H), 2.56 (s, 4H), 2.45-2.33 (m, 1H), 2.09-2.01 (m, 1H), 1.73 (s, 4H).

### Example 259:3-(5-(7-methyl-3-(oxetan-3-yl)-2-(pyrrolidin-1-ylmethyl)-3H-imidazo[4,5-b]pyridin-5-yl)-1-oxoisoindolin-2-yl) piperidine-2,6-dione

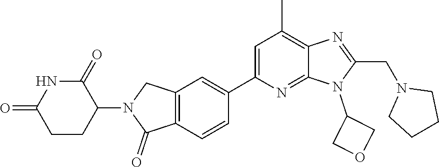 [SMILES: Cc1cc(-c2ccc3c(c2)CN(C2CCC(=O)NC2=O)C3=O)nc2c1nc(CN1CCCC1)n2C1COC1]

The title compound was prepared in a manner analogous to Example 203 by alkylation between pyrrolidine and 5-chloro-7-methyl-3-(oxetan-3-yl)-2-(pyrrolidin-1-ylmethyl)-3H-imidazo[4,5-b]pyridine (Intermediate 100b) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calcd. for C₂₈H₃₀N₆O₄, 514.23; m/z found, 515.3 [M+H]⁺. ¹HNMR (400 MHZ, DMSO-d₆) δ 11.02 (s, 1H), 8.37 (s, 1H), 8.33 (d, J=8.2 Hz, 1H), 7.88 (s, 1H), 7.85 (d, J=8.0 Hz, 1H), 5.97 (t, J=8.0 Hz, 1H), 5.62 (s, 2H), 5.16 (d, J=7.8 Hz, 1H), 4.94 (t, J=7.2 Hz, 2H), 4.58 (d, J=17.4 Hz, 1H), 4.43 (d, J=17.4 Hz, 1H), 3.92 (s, 2H), 2.93 (d, J=13.2 Hz, 1H), 2.62 (d, J=8.0 Hz, 4H), 2.43 (s, 5H), 2.06 (s, 1H), 1.71 (s, 4H).

### Example 260:3-(4-fluoro-5-(1-(1-methylazetidin-3-yl)-4-(pyrrolidin-1-ylmethyl)-1H-pyrrolo[2,3-b]pyridin-6-yl)-1-oxoisoindolin-2-yl) piperidine-2,6-dione

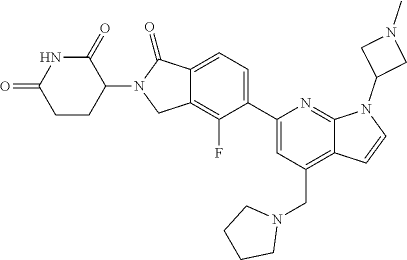 [SMILES: CN1CC(n2ccc3c(CN4CCCC4)cc(-c4ccc5c(c4F)CN(C4CCC(=O)NC4=O)C5=O)nc32)C1]

The title compound was prepared in a manner analogous to Example 1 by Suzuki coupling of chloro-1-(1-methylazetidin-3-yl)-4-(pyrrolidin-1-ylmethyl)-1H-pyrrolo[2,3-b]pyridine (Intermediate 101) with 3-(4-fluoro-1-oxo-5-(4,4,5,5-tetramethyl-1,3,2-dioxaborolan-2-yl) isoindolin-2-yl) piperidine-2,6-dione (Intermediate 14) as a brown solid. LC-MS (ESI): mass calcd. for C₂₉H₃₁FN603, 530.2; m/z found, 531.2 [M+H]⁺. ¹H NMR (400 MHz, DMSO-d₆) δ 11.04 (s, 1H), 8.25 (s, 1H), 8.20 (d, J=7.2 Hz, 1H), 7.92 (d, J=2.4 Hz, 1H), 7.72 (d, J=8.0 Hz, 1H), 7.64 (s, 1H), 6.73 (d, J=2.8 Hz, 1H), 5.52-5.39 (m, 1H), 5.19-5.14 (m, 1H), 4.66 (d, J=17.6 Hz, 1H), 4.49 (d, J=17.6 Hz, 1H), 3.95 (s, 2H), 3.83 (s, 2H), 3.57 (s, 2H), 2.92 (dd, J=12.8, 4.0 Hz, 1H), 2.67-2.60 (m, 1H), 2.57-2.52 (m, 4H), 2.48-2.44 (m, 1H), 2.42 (d, J=0.6 Hz, 3H), 2.09-2.02 (m, 1H), 1.75-1.70 (m, 4H).

### Example 261:3-(5-(1-(1-methylazetidin-3-yl)-4-(pyrrolidin-1-ylmethyl)-1H-pyrazolo[3,4-b]pyridin-6-yl)-1-oxoisoindolin-2-yl) piperidine-2,6-dione

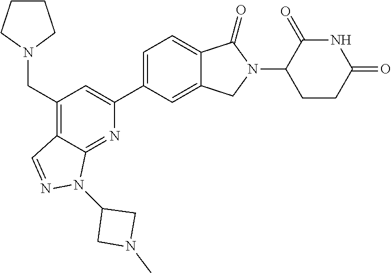 [SMILES: CN1CC(n2ncc3c(CN4CCCC4)cc(-c4ccc5c(c4)CN(C4CCC(=O)NC4=O)C5=O)nc32)C1]

The title compound was prepared in a manner analogous to Example 1 by Suzuki coupling of 66-chloro-1-(1-methylazetidin-3-yl)-4-(pyrrolidin-1-ylmethyl)-1H-pyrazolo[3,4-b]pyridine (Intermediate 102) with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₂₈H₃₁N₇O₃, 513.25; m/z found, 514.2 [M+H]⁺. ¹H NMR (400 MHZ, DMSO-d6) § 11.03 (s, 1H), 8.46 (s, 1H), 8.42 (s, 1H), 8.39 (d, J=8.4 Hz, 1H), 8.19 (s, 2H), 7.92-7.86 (m, 2H), 5.79-5.66 (m, 1H), 5.19-5.14 (m, 1H), 4.59 (d, J=17.4 Hz, 1H), 4.46 (d, J=17.4 Hz, 1H), 4.07 (s, 2H), 4.02 (t, J=7.4 Hz, 2H), 3.79 (d, J=7.4 Hz, 2H), 2.98-2.91 (m, 1H), 2.65-2.50 (m, 5H), 2.52 (s, 3H), 2.48-2.40 (m, 1H), 2.11-2.00 (m, 1H), 1.76 (s, 4H).

### Example 262:3-(5-(5-ethyl-1-(oxetan-3-yl)-4-(pyrrolidin-1-ylmethyl)-1H-pyrrolo[2,3-b]pyridin-6-yl)-1-oxoisoindolin-2-yl) piperidine-2,6-dione

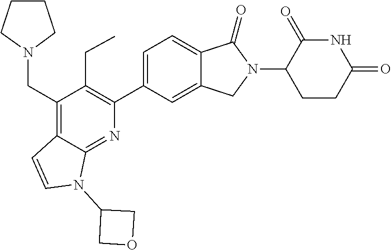 [SMILES: CCc1c(-c2ccc3c(c2)CN(C2CCC(=O)NC2=O)C3=O)nc2c(ccn2C2COC2)c1CN1CCCC1]

The title compound was prepared in a manner analogous to Example 1 by reductive amination between pyrrolidine and 6-chloro-5-ethyl-1-(oxetan-3-yl)-1H-pyrrolo[2,3-b]pyridine-4-carbaldehyde (Intermediate 103) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₃₀H₃₃N₅O₄, 527.25; m/z found, 528.2 [M+H]⁺. ¹H NMR (400 MHZ, DMSO-d₆) δ 11.02 (s, 1H), 8.15 (s, 1H), 7.95 (d, J=2.4 Hz, 1H), 7.81 (d, J=7.8 Hz, 1H), 7.66 (s, 1H), 7.56 (d, J=7.8 Hz, 1H), 6.83 (s, 1H), 5.94 -5.90 (m, 1H), 5.19-5.14 (m, 1H), 4.96 (d, J=7.2 Hz, 4H), 4.54 (d, J=17.4 Hz, 1H), 4.40 (d, J=17.4 Hz, 1H), 3.94 (s, 2H), 2.93 (dd, J=21.8, 9.0 Hz, 1H), 2.79 (d, J=7.4 Hz, 2H), 2.62 (d, J=16.8 Hz, 1H), 2.50-2.48 (m, 4H), 2.47-2.34 (m, 1H), 2.12-1.99 (m, 1H), 1.69 (s, 4H), 0.94 (t, J=7.2 Hz, 3H).

### Example 263: (S)-3-(5-(1-(1-methyl-1H-imidazol-4-yl)-4-(pyrrolidin-1-ylmethyl)-1H-pyrrolo[2,3-b]pyridin-6-yl)-1-oxoisoindolin-2-yl) piperidine-2,6-dione

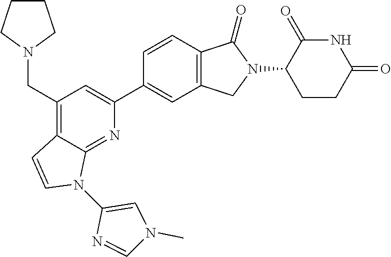 [SMILES: Cn1cnc(-n2ccc3c(CN4CCCC4)cc(-c4ccc5c(c4)CN([C@H]4CCC(=O)NC4=O)C5=O)nc32)c1]

### Step A: 6-chloro-1-(1-methyl-1H-imidazol-4-yl)-4-(pyrrolidin-1-ylmethyl)-1H-pyrrolo[2,3-b]pyridine

To a solution of 6-chloro-4-(pyrrolidin-1-ylmethyl)-1H-pyrrolo[2,3-b]pyridine (Intermediate 31, 100 mg, 424 μmol, 1.0 eq) in 1,4-Dioxane (7.00 mL) were added 4-iodo-1-methyl-1H-imidazole (132 mg, 636 μmol, 1.5 eq), Potassium phosphate tribasic (180 mg, 849 μmol, 2.0 eq), (1S,2S)-cyclohexane-1,2-diamine (9.69 mg, 84.9 μmol, 0.2 eq), and CuI (8.08 mg, 42.4 μmol, 0.1 eq). The mixture was stirred under N₂ at 110° C. for 16 h. After cooled to room temperature, the reaction mixture was quenched with water (10 mL) and extracted with DCM (10 mL×3). The organic layer was dried over anhydrous Na₂SO₄, filtered and concentrated under reduced pressure. The crude product was purified by Prep-TLC (DCM/MeOH=10/1 v/v) to afford 6-chloro-1-(1-methyl-1H-imidazol-4-yl)-4-(pyrrolidin-1-ylmethyl)-1H-pyrrolo[2,3-b]pyridine (130 mg, yield 97%) as a yellow solid. LC-MS (ESI): mass calced for: C₁₆H₁₈ClN₅, 315.81; m/z found, 316.2 [M+H]⁺.

### Step B: tert-butyl(S)-5-amino-4-(5-(1-(1-methyl-1H-imidazol-4-yl)-4-(pyrrolidin-1-ylmethyl)-1H-pyrrolo[2,3-b]pyridin-6-yl)-1-oxoisoindolin-2-yl)-5-oxopentanoate

To a solution of 6-chloro-1-(1-methyl-1H-imidazol-4-yl)-4-(pyrrolidin-1-ylmethyl)-1H-pyrrolo[2,3-b]pyridine (130 mg, 412 μmol, 1.0 eq) in 1,4-Dioxane (5.00 mL) and water (0.50 mL) were added tert-butyl (R)-5-amino-5-oxo-4-(1-oxo-5-(4,4,5,5-tetramethyl-1,3,2-dioxaborolan-2-yl) isoindolin-2-yl) pentanoate (Intermediate 33, 274 mg, 617 μmol, 1.5 eq), Potassium phosphate tribasic (262 mg, 1.23 mmol, 3.0 eq), and 1,1′-Bis(di-t-butylphosphino) ferrocene palladium dichloride (26.8 mg, 41.2 μmol, 0.1 eq). The mixture was stirred under N₂ at 95° C. for 1 h. After cooled to room temperature, the reaction mixture was filtered and the filtrate was concentrated in vacuum. The crude product was purified by Prep-TLC(DCM/MeOH=15/1 v/v) to give tert-butyl(S)-5-amino-4-(5-(1-(1-methyl-1H-imidazol-4-yl)-4-(pyrrolidin-1-ylmethyl)-1H-pyrrolo[2,3-b]pyridin-6-yl)-1-oxoisoindolin-2-yl)-5-oxopentanoate (160 mg, yield 65%) as a yellow solid. LC-MS (ESI): mass calced for: C₃₃H₃₉N₇O₄, 597.72; m/z found, 598.1 [M+H]⁺.

### Step C: (S)-3-(5-(1-(1-methyl-1H-imidazol-4-yl)-4-(pyrrolidin-1-ylmethyl)-1H-pyrrolo[2,3-b]pyridin-6-yl)-1-oxoisoindolin-2-yl) piperidine-2,6-dione

To a solution of tert-butyl (R)-5-amino-4-(5-(1-(1-methyl-1H-imidazol-4-yl)-4-(pyrrolidin-1-ylmethyl)-1H-pyrrolo[2,3-b]pyridin-6-yl)-1-oxoisoindolin-2-yl)-5-oxopentanoate (160 mg, 268 μmol, 1.0 eq) in ACN (4.00 mL) was added with benzenesulfonic acid (127 mg, 803 μmol, 3.0 eq). The mixture was stirred at 95° C. for 8 h. After cooled to room temperature, the reaction mixture was filtered and the filtrate was concentrated in vacuum. The crude product was purified by Prep-HPLC with YMC-Actus Triart 18C(5 μm, 20× 250 mm), and mobile phase of 5-99% ACN in water (0.1% FA) over 10 min and then hold at 100% ACN for 2 min, at a flow rate of 25 mL/min to give(S)-3-(5-(1-(1-methyl-1H-imidazol-4-yl)-4-(pyrrolidin-1-ylmethyl)-1H-pyrrolo[2,3-b]pyridin-6-yl)-1-oxoisoindolin-2-yl) piperidine-2,6-dione (72.0 mg, yield 51%) as a yellow solid. LC-MS (ESI): mass calced for: C₂₉H₂₉N₇O₃, 523.6; m/z found, 524.4 [M+H]⁺. ¹H NMR (400 MHZ, DMSO-d₆) δ 11.03 (s, 1H), 8.43 (s, 1H), 8.36 (d, J=7.8 Hz, 1H), 8.14 (s, 1H), 7.89 (d, J=8.0 Hz, 1H), 7.67 (s, 1H), 7.60 (d, J=6.4 Hz, 1H), 7.31 (d, J=5.6 Hz, 1H), 6.92 (s, 1H), 5.20-5.15 (m, 1H), 4.60 (d, J=17.2 Hz, 1H), 4.49 (d, J=17.2 Hz, 1H), 4.33 (s, 2H), 3.82 (s, 3H), 2.98-2.90 (m, 5H), 2.63 (d, J=17.2 Hz, 1H), 2.49-2.38 (m, 1H), 2.10-2.01 (m, 1H), 1.85 (s, 4H).

### Example 264:3-(5-(4-((4-azaspiro[2.5]octan-4-yl)methyl)-1-(oxetan-3-yl)-1H-pyrrolo[2,3-b]pyridin-6-yl)-1-oxoisoindolin-2-yl) piperidine-2,6-dione

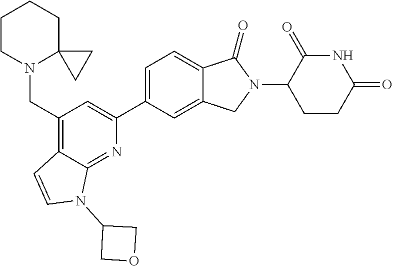 [SMILES: O=C1CCC(N2Cc3cc(-c4cc(CN5CCCCC56CC6)c5ccn(C6COC6)c5n4)ccc3C2=O)C(=O)N1]

The title compound was prepared in a manner analogous to Example 1 by reductive amination between 4-azaspiro[2.5]octane hydrochloride and 6-chloro-1-(oxetan-3-yl)-1H-pyrrolo[2,3-b]pyridine-4-carbaldehyde (Intermediate 29) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₃₁H₃₃N₅O₄, 539.25; m/z found, 540.3 [M+H]⁺. ¹H NMR (400 MHZ, DMSO-d₆) δ 11.01 (s, 1H), 8.36 (s, 1H), 8.31 (d, J=7.6 Hz, 1H), 7.96 (s, 1H), 7.83 (d, J=8.0 Hz, 1H), 7.75 (s, 1H), 6.72 (s, 1H), 6.17-6.06 (m, 1H), 5.18-5.13 (m, 1H), 5.06 (d, J=7.4 Hz, 4H), 4.57 (d, J=16.6 Hz, 1H), 4.44 (d, J=16.4 Hz, 1H), 4.18 (s, 2H), 2.99-2.87 (m, 1H), 2.67-2.60 (m, 3H), 2.46-2.41 (m, 1H), 2.13-1.99 (m, 1H), 1.69 (d, J=4.0 Hz, 2H), 1.52-1.49 (m, 4H), 0.65 (s, 2H), 0.42 (s, 2H).

### Example 265: (S)-3-(5-(1-(1-methyl-1H-pyrazol-3-yl)-4-(pyrrolidin-1-ylmethyl)-1H-pyrrolo[2,3-b]pyridin-6-yl)-1-oxoisoindolin-2-yl) piperidine-2,6-dione

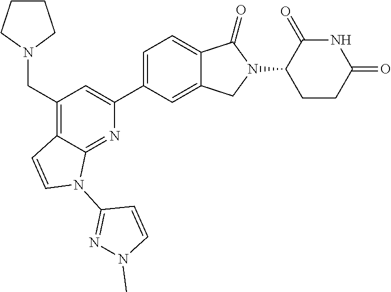 [SMILES: Cn1ccc(-n2ccc3c(CN4CCCC4)cc(-c4ccc5c(c4)CN([C@H]4CCC(=O)NC4=O)C5=O)nc32)n1]

The title compound was prepared in a manner analogous to Example 263 by Ullman coupling of 3-iodo-1-methyl-1H-pyrazole with 6-chloro-4-(pyrrolidin-1-ylmethyl)-1H-pyrrolo[2,3-b]pyridine (Intermediate 31) followed by Suzuki coupling with tert-butyl(S)-5-amino-5-oxo-4-(1-oxo-5-(4,4,5,5-tetramethyl-1,3,2-dioxaborolan-2-yl) isoindolin-2-yl) pentanoate (Intermediate 33) and acid-catalyzed cyclization with benzenesulfonic acid as a white solid. LC-MS (ESI): mass calcd. for C₂₉H₂₉N₇O₃, 523.2; m/z found, 524.2 [M+H]⁺. ¹H NMR (400 MHZ, DMSO-d₆) δ 11.03 (s, 1H), 8.39 (s, 1H), 8.34 (d, J=8.0 Hz, 1H), 8.09 (s, 1H), 8.03 (s, 1H), 7.89 (s, 1H), 7.61 (s, 1H), 7.31 (s, 1H), 7.20 (s, 1H), 6.99 (s, 1H), 5.19-5.15 (m, 1H), 4.60 (d, J=17.2 Hz, 1H), 4.46 (d, J=17.2 Hz, 1H), 4.44 (s, 2H), 3.92 (s, 3H), 3.04-2.92 (m, 5H), 2.63 (d, J=16.4 Hz, 1H), 2.43 (t, J=17.6 Hz, 1H), 2.06 (t, J=12.2 Hz, 1H), 1.94-1.82 (m, 4H).

### Example 266:3-(5-(1-(1,1-dioxidothietan-3-yl)-4-((4-methoxypiperidin-1-yl)methyl)-1H-pyrrolo[2,3-b]pyridin-6-yl)-4-fluoro-1-oxoisoindolin-2-yl) piperidine-2,6-dione

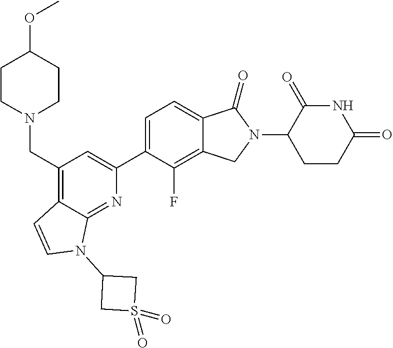 [SMILES: COC1CCN(Cc2cc(-c3ccc4c(c3F)CN(C3CCC(=O)NC3=O)C4=O)nc3c2ccn3C2CS(=O)(=O)C2)CC1]

### Step A: 6-chloro-4-((4-methoxypiperidin-1-yl)methyl)-1H-pyrrolo[2,3-b]pyridine

To a solution of 6-chloro-1H-pyrrolo[2,3-b]pyridine-4-carbaldehyde (Intermediate 9, 200 mg, 1.11 mmol, 1.0 eq) and 4-methoxypiperidine (191 mg, 1.66 mmol, 1.5 eq) in DCM (10.0 mL) and MeOH (1.00 mL) was added with acetic acid (333 mg, 318 μL, 5.54 mmol, 5.0 eq). The reaction mixture was stirred at room temperature for 18 h. Then Sodium triacetoxyborohydride (469 mg, 2.21 mmol, 2.0 eq) was added to above mixture and the reaction mixture was stirred at room temperature for 2 h. The mixture was filtered and the filtrate was concentrated under reduced pressure. The crude product was purified by flash column chromatography on silica gel (DCM/MeOH=60/1 v/v) to give 6-chloro-4-((4-methoxypiperidin-1-yl)methyl)-1H-pyrrolo[2,3-b]pyridine (114 mg, yield 37%) as a white solid. LC-MS (ESI): mass calced for: C₁₄H₁₈ClN₃O, 279.11; m/z found, 280.1 [M+H]⁺.

### Step B: 3-(6-chloro-4-((4-methoxypiperidin-1-yl)methyl)-1H-pyrrolo[2,3-b]pyridin-1-yl) thietane 1,1-dioxide

To a solution of 6-chloro-4-((4-methoxypiperidin-1-yl)methyl)-1H-pyrrolo[2,3-b]pyridine (114 mg, 407 μmol, 1.0 eq) in DMF (6 mL) was added Sodium hydride (60% suspended in oil) (24.4 mg, 611 μmol, 1.5 eq) at 0° C. and the mixture was stirred for 30 min at 0° C. Then a solution of 3-bromothietane 1,1-dioxide (98.0 mg, 530 μmol, 1.3 eq) in DMF (4 mL) was dropwise added to above mixture at 0° C. and the reaction mixture was stirring for 30 min at 0° C. The mixture was quenched with ice-water (10 mL) and extracted with EtOAc (20 mL×3). The organic layers were washed with brine (20 mL×4), dried over anhydrous Na₂SO₄, filtered and concentrated in vacuum. The crude product was purified by Prep-TLC (DCM/MeOH=10/1 v/v) to give 3-(6-chloro-4-((4-methoxypiperidin-1-yl)methyl)-1H-pyrrolo[2,3-b]pyridin-1-yl) thietane 1,1-dioxide (120 mg, yield 77%) as a yellow solid. LC-MS (ESI): mass calced for: C₁₇H₂₂ClN₃O₃S, 383.11; m/z found, 384.1 [M+H]⁺. ¹H NMR (400 MHz, DMSO-d₆) δ 7.84 (d, J=2.8 Hz, 1H), 7.16 (s, 1H), 6.80 (d, J=2.8 Hz, 1H), 5.68-5.61 (m, 1H), 4.90 (dd, J=13.8, 9.6 Hz, 2H), 4.70 (dd, J=14.4, 3.8 Hz, 2H), 3.75 (s, 2H), 3.21 (s, 3H), 3.18-3.14 (m, 1H), 2.65 (d, J=4.6 Hz, 2H), 2.16 (t, J=9.8 Hz, 2H), 1.82 (d, J=10.6 Hz, 2H), 1.48-1.41 (m, 2H).

### Step C: 3-(5-(1-(1,1-dioxidothietan-3-yl)-4-((4-methoxypiperidin-1-yl)methyl)-1H-pyrrolo[2,3-b]pyridin-6-yl)-4-fluoro-1-oxoisoindolin-2-yl) piperidine-2,6-dione

To a suspension of 3-(6-chloro-4-((4-methoxypiperidin-1-yl)methyl)-1H-pyrrolo[2,3-b]pyridin-1-yl) thietane 1,1-dioxide (20.0 mg, 52.1 μmol, 1.0 eq), 3-(4-fluoro-1-oxo-5-(4,4,5,5-tetramethyl-1,3,2-dioxaborolan-2-yl) isoindolin-2-yl) piperidine-2,6-dione (Intermediate 14, 30.3 mg, 78.1 μmol, 1.5 eq), and Potassium phosphate tribasic (33.2 mg, 156 μmol, 3.0 eq) in 1,4-Dioxane (4.00 mL) and Water (0.40 mL) was added 1,1′-Bis(di-t-butylphosphino) ferrocene palladium dichloride (3.40 mg, 5.21 μmol, 0.1 eq). The reaction mixture was stirred under nitrogen at 95° C. for 1 h. After cooled to room temperature, the mixture was filtered and the filtrate was concentrated in vacuum. The crude product was purified by Prep-TLC(DCM/MeOH=9/1 v/v) to give impure product as a yellow solid. The impure product was further purified by Prep-HPLC with Kromasil Eternity XT C₁₈ (10 μm, 21.2×250 mm), and mobile phase of 5-95% ACN in water (0.1% FA) over 10 min and then hold at 100% ACN for 3 min, at a flow rate of 30 mL/min to give 3-(5-(1-(1,1-dioxidothietan-3-yl)-4-((4-methoxypiperidin-1-yl)methyl)-1H-pyrrolo[2,3-b]pyridin-6-yl)-4-fluoro-1-oxoisoindolin-2-yl) piperidine-2,6-dione formate (9.90 mg, yield 31%) as a white solid. LC-MS (ESI): mass calced for: C₃₀H₃₂FN506S, 609.21; m/z found, 610.2 [M+H]⁺. ¹H NMR (400 MHZ, DMSO-d₆) δ 11.03 (s, 1H), 8.29 (t, J=7.6 Hz, 1H), 8.13 (s, 1H), 7.89 (s, 1H), 7.71 (s, 2H), 6.81 (s, 1H), 5.84-5.73 (m, 1H), 5.18-5.13 (m, 1H), 4.89 (d, J=7.4 Hz, 4H), 4.67 (d, J=17.4 Hz, 1H), 4.50 (d, J=17.4 Hz, 1H), 3.85 (s, 2H), 3.22 (s, 4H), 2.94 (t, J=13.4 Hz, 1H), 2.73 (s, 2H), 2.63 (d, J=16.4 Hz, 1H), 2.47 (s, 1H), 2.21 (s, 2H), 2.08-2.03 (m, 1H), 1.86-1.82 (m, 2H), 1.50-1.45 (m, 2H). ¹⁹F NMR (400 MHZ, DMSO-d₆) δ-123.94 (ppm).

### Example 267: (S)-3-(5-(1-(oxazol-2-yl)-4-(pyrrolidin-1-ylmethyl)-1H-pyrrolo[2,3-b]pyridin-6-yl)-1-oxoisoindolin-2-yl) piperidine-2,6-dione

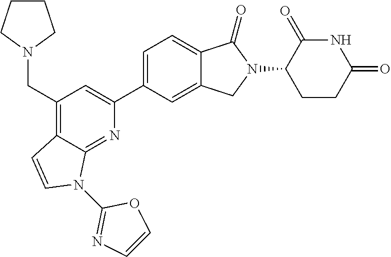 [SMILES: O=C1CC[C@H](N2Cc3cc(-c4cc(CN5CCCC5)c5ccn(-c6ncco6)c5n4)ccc3C2=O)C(=O)N1]

The title compound was prepared in a manner analogous to Example 263 by Ullman coupling of 2-iodooxazole with 6-chloro-4-(pyrrolidin-1-ylmethyl)-1H-pyrrolo[2,3-b]pyridine (Intermediate 31) followed by Suzuki coupling with tert-butyl(S)-5-amino-5-oxo-4-(1-oxo-5-(4,4,5,5-tetramethyl-1,3,2-dioxaborolan-2-yl) isoindolin-2-yl) pentanoate (Intermediate 33) and acid-catalyzed cyclization with benzenesulfonic acid as a white solid. LC-MS (ESI): mass calced for: C₂₈H₂₆N₆O₄, 510.20; m/z found, 511.2 [M+H]⁺. ¹H NMR (400 MHZ, DMSO-d₆) δ 11.03 (s, 1H), 10.29 (s, 1H), 8.41-8.32 (m, 2H), 8.24 (d, J=13.6 Hz, 2H), 8.15 (s, 1H), 7.92 (d, J=8.0 Hz, 1H), 7.44 (s, 1H), 7.21 (s, 1H), 5.17-5.12 (m, 1H), 4.81 (s, 2H), 4.60 (d, J=17.2 Hz, 1H), 4.47 (d, J=17.2 Hz, 1H), 3.62 (s, 2H), 3.25 (s, 2H), 2.93 (t, J=12.8 Hz, 1H), 2.64 (d, J=16.6 Hz, 1H), 2.48-2.38 (m, 1H), 2.07 (d, J=7.4 Hz, 3H), 1.92 (s, 2H). ¹⁹F NMR (376 MHz, DMSO) δ -73.73 (ppm).

### Example 268:3-(5-(1-(1,1-dioxidothietan-3-yl)-4-((4-fluoropiperidin-1-yl)methyl)-1H-pyrrolo[2,3-b]pyridin-6-yl)-4-fluoro-1-oxoisoindolin-2-yl) piperidine-2,6-dione

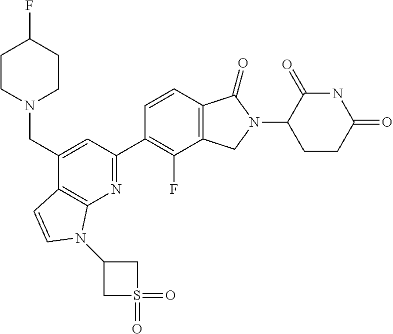 [SMILES: O=C1CCC(N2Cc3c(ccc(-c4cc(CN5CCC(F)CC5)c5ccn(C6CS(=O)(=O)C6)c5n4)c3F)C2=O)C(=O)N1]

The title compound was prepared in a manner analogous to Example 266 by reductive amination between 4-fluoropiperidine and 6-chloro-1H-pyrrolo[2,3-b]pyridine-4-carbaldehyde (Intermediate 9) followed by displacement with 3-bromothietane 1,1-dioxide and Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14) as a white solid. LC-MS: 598 (M+H)+. Revised as the following: LC-MS (ESI): mass calced for: C₂₉H₂₉F₂N₅O₅S, 597.1; m/z found, 598.1 [M+H]⁺. ¹H NMR (400 MHZ, DMSO-d₆) δ 11.05 (s, 1H), 8.29 (t, J=7.2 Hz, 1H), 8.14 (s, 1H), 7.90 (s, 1H), 7.71 (s, 2H), 6.83 (s, 1H), 5.82-5.73 (m, 1H), 5.17 (dd, J=13.2, 4.6 Hz, 1H), 4.89 (d, J=7.4 Hz, 4H), 4.81-4.61 (m, 2H), 4.50 (d, J=17.2 Hz, 1H), 3.87 (s, 2H), 2.95 (t, J=12.89 Hz, 1H), 2.62 (d, J=13.6 Hz, 3H), 2.44 (d, J=24.4 Hz, 3H), 2.10-2.00 (m, 1H), 1.87 (d, J=17.4 Hz, 2H), 1.75 (s, 2H). ¹⁹F NMR (400 MHZ, DMSO-d₆) δ-123.95 (ppm).

### Example 269:3-(5-(1-(1,1-dioxidothietan-3-yl)-4-((tetrahydro-1H-furo[3,4-c]pyrrol-5 (3H)-yl)methyl)-1H-pyrrolo[2,3-b]pyridin-6-yl)-4-fluoro-1-oxoisoindolin-2-yl) piperidine-2,6-dione

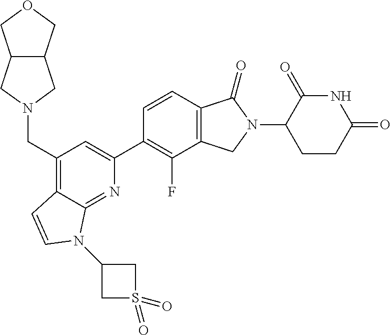 [SMILES: O=C1CCC(N2Cc3c(ccc(-c4cc(CN5CC6COCC6C5)c5ccn(C6CS(=O)(=O)C6)c5n4)c3F)C2=O)C(=O)N1]

The title compound was prepared in a manner analogous to Example 266 by reductive amination between hexahydro-1H-furo[3,4-c]pyrrole and 6-chloro-1H-pyrrolo[2,3-b]pyridine-4-carbaldehyde (Intermediate 9) followed by displacement with 3-bromothietane 1,1-dioxide and Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14) as a white solid. LC-MS (ESI): mass calced for: C₃₀H₃₀FNsO6S, 607.66; m/z found, 608.2 [M+H]⁺. ¹H NMR (400 MHZ, DMSO-d₆) δ 11.03 (s, 1H), 8.30 (t, J=6.8 Hz, 1H), 7.90 (s, 1H), 7.71 (d, J=8.0 Hz, 2H), 6.80 (s, 1H), 5.82-5.75 (m, 1H), 5.19-5.14 (m, 1H), 4.89 (d, J=7.4 Hz, 4H), 4.67 (d, J=17.2 Hz, 1H), 4.50 (d, J=17.2 Hz, 1H), 3.92 (s, 2H), 3.78 (s, 2H), 3.40 (s, 2H), 3.01-2.89 (m, 1H), 2.73 (s, 2H), 2.63 (d, J=17.6 Hz, 1H), 2.54 (d, J=2.6 Hz, 3H), 2.46 (d, J=11.8 Hz, 2H), 2.11-2.00 (m, 1H). ¹⁹F NMR (400 MHZ, DMSO-d₆) δ-124.02 (ppm).

### Example 270:3-(5-(5-chloro-1-(oxetan-3-yl)-4-(pyrrolidin-1-ylmethyl)-1H-pyrrolo[2,3-b]pyridin-6-yl)-4-fluoro-1-oxoisoindolin-2-yl) piperidine-2,6-dione

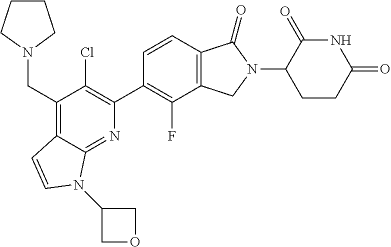 [SMILES: O=C1CCC(N2Cc3c(ccc(-c4nc5c(ccn5C5COC5)c(CN5CCCC5)c4Cl)c3F)C2=O)C(=O)N1]

The title compound was prepared in a manner analogous to Example 1 by Suzuki coupling of 5,6-dichloro-1-(oxetan-3-yl)-4-(pyrrolidin-1-ylmethyl)-1H-pyrrolo[2,3-b]pyridine (Intermediate 104) with 3-(4-fluoro-1-oxo-5-(4,4,5,5-tetramethyl-1,3,2-dioxaborolan-2-yl) isoindolin-2-yl) piperidine-2,6-dione (Intermediate 14) as a white solid. LC-MS (ESI): mass calced for: C₂₈H₂₇CIFNs04, 552.00; m/z found, 552.2 [M+H]⁺. ¹H NMR (400 MHZ, DMSO-d6) § 11.05 (s, 1H), 9.91 (s, 1H), 8.36 (s, 1H), 7.76 (d, J=7.6 Hz, 1H), 7.68 (t, J=6.4 Hz, 1H), 7.14 (s, 1H), 6.02-5.91 (m, 1H), 5.20-5.16 (m, 1H), 5.04-4.96 (m, 4H), 4.90 (s, 2H), 4.68 (d, J=17.4 Hz, 1H), 4.49 (d, J=17.4 Hz, 1H), 3.56 (s, 2H), 3.32 (s, 2H), 2.95 (t, J=13.0 Hz, 1H), 2.63 (d, J=17.0 Hz, 1H), 2.44 (d, J=13.4 Hz, 1H), 2.09 (d, J=1.8 Hz, 3H), 1.89 (d, J=4.6 Hz, 2H). ¹⁹F NMR (400 MHZ, DMSO-d₆) δ-73.80,-121.53 (ppm).

### Example 271: (S)-3-(5-(1-(1H-pyrazol-4-yl)-4-(pyrrolidin-1-ylmethyl)-1H-pyrrolo[2,3-b]pyridin-6-yl)-1-oxoisoindolin-2-yl) piperidine-2,6-dione

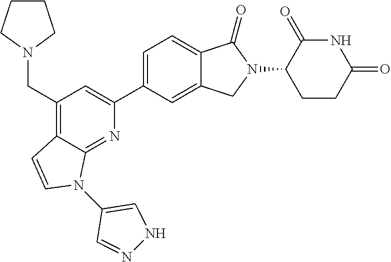 [SMILES: O=C1CC[C@H](N2Cc3cc(-c4cc(CN5CCCC5)c5ccn(-c6cn[nH]c6)c5n4)ccc3C2=O)C(=O)N1]

The title compound was prepared in a manner analogous to Example 263 by Ullman coupling of 4-iodo-1-(tetrahydro-2H-pyran-2-yl)-1H-pyrazole with 6-chloro-4-(pyrrolidin-1-ylmethyl)-1H-pyrrolo[2,3-b]pyridine (Intermediate 31) followed by Suzuki coupling with tert-butyl(S)-5-amino-5-oxo-4-(1-oxo-5-(4,4,5,5-tetramethyl-1,3,2-dioxaborolan-2-yl) isoindolin-2-yl) pentanoate (Intermediate 33) and acid-catalyzed cyclization with benzenesulfonic acid as a white solid. LC-MS (ESI): mass calced for: C₂₈H₂₇N₇O₃, 509.22; m/z found, 510.2 [M+H]⁺. ¹H NMR (400 MHz, DMSO-d₆) δ 12.96 (s, 1H), 11.02 (s, 1H), 8.39 (s, 2H), 8.34 (d, J=8.0 Hz, 1H), 8.14 (s, 1H), 7.97 (s, 1H), 7.87 (d, J=7.2 Hz, 2H), 6.88 (s, 1H), 5.19-5.15 (m, 1H), 4.59 (d, J=17.4 Hz, 1H), 4.46 (d, J=17.4 Hz, 1H), 4.09 (s, 2H), 2.94 (t, J=14.0 Hz, 1H), 2.64 (d, J=24.8 Hz, 5H), 2.48-2.36 (m, 1H), 2.10-2.01 (m, 1H), 1.78 (s, 4H).

### Example 272:3-(5-(4-((2-oxa-7-azaspiro[3.5]nonan-7-yl)methyl)-1-(1,1-dioxidothietan-3-yl)-1H-pyrrolo[2,3-b]pyridin-6-yl)-4-fluoro-1-oxoisoindolin-2-yl) piperidine-2,6-dione

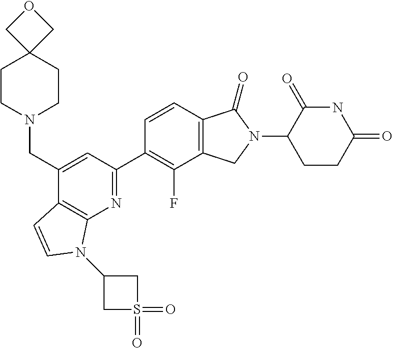 [SMILES: O=C1CCC(N2Cc3c(ccc(-c4cc(CN5CCC6(CC5)COC6)c5ccn(C6CS(=O)(=O)C6)c5n4)c3F)C2=O)C(=O)N1]

The title compound was prepared in a manner analogous to Example 266 by reductive amination between 2-oxa-7-azaspiro[3.5]nonane and 6-chloro-1H-pyrrolo[2,3-b]pyridine-4-carbaldehyde (Intermediate 9) followed by displacement with 3-bromothietane 1,1-dioxide and Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14) as a yellow solid. LC-MS (ESI): mass calced for: C₃₁H₃₂FN5O₆S, 621.2; m/z found, 622.1 [M+H]⁺. ¹H NMR (400 MHZ, DMSO-d₆) δ 11.04 (s, 1H), 8.29 (t, J=7.2 Hz, 1H), 8.19 (s, 1H), 7.89 (s, 1H), 7.70 (d, J=6.8 Hz, 2H), 6.79 (s, 1H), 5.82-5.72 (m, 1H), 5.17 (d, J=8.2 Hz, 1H), 4.89 (d, J=7.2 Hz, 4H), 4.66 (d, J=17.4 Hz, 1H), 4.50 (d, J=17.4 Hz, 1H), 4.27 (s, 4H), 3.80 (s, 2H), 2.98-2.91 (m, 1H), 2.63 (d, J=15.6 Hz, 1H), 2.45 (d, J=13.4 Hz, 1H), 2.35 (s, 4H), 2.10-2.02 (m, 1H), 1.80 (s, 4H). ¹⁹F NMR (400 MHZ, DMSO-d₆) δ-123.95 (ppm).

### Example 273:3-(6-chloro-5-(1-(oxetan-3-yl)-4-(pyrrolidin-1-ylmethyl)-1H-pyrrolo[2,3-b]pyridin-6-yl)-1-oxoisoindolin-2-yl) piperidine-2,6-dione

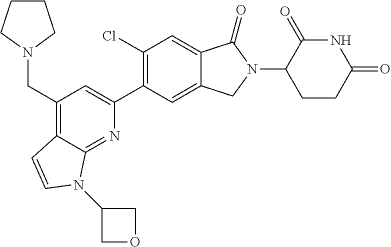 [SMILES: O=C1CCC(N2Cc3cc(-c4cc(CN5CCCC5)c5ccn(C6COC6)c5n4)c(Cl)cc3C2=O)C(=O)N1]

The title compound was prepared in a manner analogous to Example 1 by reductive amination between pyrrolidine and 6-chloro-1-(oxetan-3-yl)-1H-pyrrolo[2,3-b]pyridine-4-carbaldehyde (Intermediate 29) followed by Suzuki coupling with 3-(6-chloro-1-oxo-5-(4,4,5,5-tetramethyl-1,3,2-dioxaborolan-2-yl) isoindolin-2-yl) piperidine-2,6-dione (Intermediate 34) as a yellow solid. LC-MS (ESI): mass calced for: C₂₈H₂₈ClN₅O₄, 533.2; m/z found, 534.2 [M+H]⁺. ¹H NMR (400 MHZ, DMSO-d₆) δ 11.03 (s, 1H), 8.14 (s, 1H), 8.05 (s, 1H), 7.89 (s, 2H), 7.52 (s, 1H), 6.85 (s, 1H), 6.05-5.95 (m, 1H), 5.16 (d, J=9.4 Hz, 1H), 5.02 (dd, J=16.0, 7.2 Hz, 4H), 4.55 (d, J=17.6 Hz, 1H), 4.42 (d, J=17.6 Hz, 1H), 4.17 (s, 2H), 2.92 (d, J=13.4 Hz, 1H), 2.74 (s, 4H), 2.62 (d, J=15.8 Hz, 1H), 2.43 (d, J=13.0 Hz, 1H), 2.11-2.00 (m, 1H), 1.79 (s, 4H).

### Example 274:3-(5-(1-(3,3-difluorocyclobutyl)-4-(pyrrolidin-1-ylmethyl)-1H-pyrrolo[2,3-b]pyridin-6-yl)-1-oxoisoindolin-2-yl) piperidine-2,6-dione

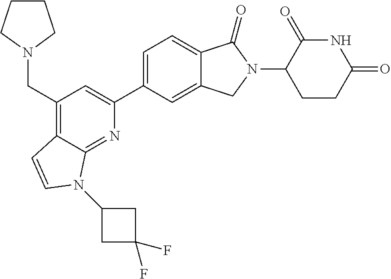 [SMILES: O=C1CCC(N2Cc3cc(-c4cc(CN5CCCC5)c5ccn(C6CC(F)(F)C6)c5n4)ccc3C2=O)C(=O)N1]

The title compound was prepared in a manner analogous to Example 1 by reductive amination between pyrrolidine and 6-chloro-1-(3,3-difluorocyclobutyl)-1H-pyrrolo[2,3-b]pyridine-4-carbaldehyde (Intermediate 105) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₉H₂₉F₂N₅O₃, 533.22; m/z found, 534.3 [M+H]⁺. ¹H NMR (400 MHZ, DMSO-d₆) δ 11.01 (s, 1H), 8.39 (s, 1H), 8.33 (d, J=8.0 Hz, 1H), 8.14 (s, 1H), 7.83 (t, J=10.0 Hz, 3H), 6.73 (s, 1H), 5.37-5.27 (m, 1H), 5.18-5.14 (m, 1H), 4.57 (d, J=17.2 Hz, 1H), 4.44 (d, J=17.2 Hz, 1H), 4.02 (s, 2H), 3.48-3.41 (m, 2H), 3.28-3.20 (m, 2H), 2.94 (t, J=14.2 Hz, 1H), 2.63 (d, J=13.2 Hz, 5H), 2.45-2.42 (m, 1H), 2.06-2.02 (m, 1H), 1.76 (s, 4H). ¹⁹F NMR (400 MHZ, DMSO-d₆) δ-82.4-82.9; 98.1-98.6 (ppm).

### Example 275:3-(5-(2-methyl-1-(oxetan-3-yl)-4-(pyrrolidin-1-ylmethyl)-1H-pyrrolo[2,3-b]pyridin-6-yl)-1-oxoisoindolin-2-yl) piperidine-2,6-dione

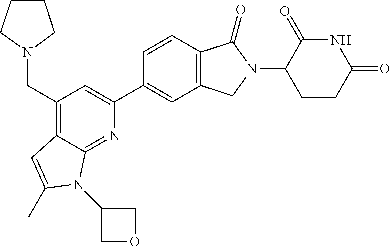 [SMILES: Cc1cc2c(CN3CCCC3)cc(-c3ccc4c(c3)CN(C3CCC(=O)NC3=O)C4=O)nc2n1C1COC1]

The title compound was prepared in a manner analogous to Example 1 by reductive amination between pyrrolidine and 6-chloro-2-methyl-1-(oxetan-3-yl)-1H-pyrrolo[2,3-b]pyridine-4-carbaldehyde (Intermediate 106) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a white solid. LC-MS (ESI): mass calced for: C₂₉H₃₁N₅O₄ 513.6; m/z found, 514.3 [M+H]⁺. ¹H NMR (400 MHZ, DMSO-d₆) δ 11.02 (s, 1H), 8.36 (s, 1H), 8.33 (d, J=8.0 Hz, 1H), 8.16 (s, 1H), 7.84 (d, J=7.8 Hz, 1H), 7.78 (s, 1H), 6.47 (s, 1H), 5.87-5.75 (m, 1H), 5.55 (s, 2H), 5.14 (d, J=13.2 Hz, 1H), 5.02 (s, 2H), 4.57 (d, J=17.4 Hz, 1H), 4.43 (d, J=17.2 Hz, 1H), 3.96 (s, 2H), 2.93 (t, J=13.4 Hz, 1H), 2.67 (d, J=10.2 Hz, 1H), 2.60 (s, 5H), 2.49 (s, 2H), 2.44 (d, J=10.8 Hz, 1H), 2.10-2.01 (m, 1H), 1.75 (s, 4H).

### Example 276:3-(3-methyl-5-(1-(oxetan-3-yl)-4-(pyrrolidin-1-ylmethyl)-1H-pyrrolo[2,3-b]pyridin-6-yl)-1-oxoisoindolin-2-yl) piperidine-2,6-dione

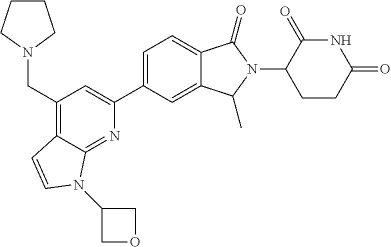 [SMILES: CC1c2cc(-c3cc(CN4CCCC4)c4ccn(C5COC5)c4n3)ccc2C(=O)N1C1CCC(=O)NC1=O]

The title compound was prepared in a manner analogous to Example 1 by reductive amination between pyrrolidine and 6-chloro-1-(oxetan-3-yl)-1H-pyrrolo[2,3-b]pyridine-4-carbaldehyde (Intermediate 29) followed by Suzuki coupling with 3-(3-methyl-1-oxo-5-(4,4,5,5-tetramethyl-1,3,2-dioxaborolan-2-yl) isoindolin-2-yl) piperidine-2,6-dione (Intermediate 28) as a white solid. LC-MS (ESI): mass calced for: C₂₉H₃₁N₅O₄, 513.24; m/z found, 514.3 [M+H]⁺. ¹H NMR (400 MHZ, DMSO-d₆) δ 10.95 (d, J=12.8 Hz, 1H), 8.39 (s, 1H), 8.32 (d, J=7.8 Hz, 1H), 8.17 (s, 1H), 7.99 (d, J=3.6 Hz, 1H), 7.82 (s, 1H), 7.79-7.75 (m, 1H), 6.78 (d, J=3.6 Hz, 1H), 6.20-6.10 (m, 1H), 5.08 (t, J=7.2 Hz, 4H), 4.89-4.75 (m, 2H), 3.98 (s, 2H), 2.90-2.80 (m, 1H), 2.65-2.62 (m, 2H), 2.56 (s, 4H), 2.07-2.02 (m, 1H), 1.74 (s, 4H), 1.56-1.52 (m, 3H).

### Example 277:3-(5-(5-chloro-1-methyl-4-(pyrrolidin-1-ylmethyl)-1H-pyrrolo[2,3-b]pyridin-6-yl)-4-fluoro-1-oxoisoindolin-2-yl) piperidine-2,6-dione

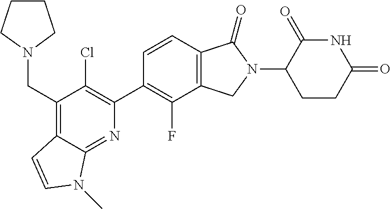 [SMILES: Cn1ccc2c(CN3CCCC3)c(Cl)c(-c3ccc4c(c3F)CN(C3CCC(=O)NC3=O)C4=O)nc21]

The title compound was prepared in a manner analogous to Example 1 by reductive amination between pyrrolidine and 5,6-dichloro-1-methyl-1H-pyrrolo[2,3-b]pyridine-4-carbaldehyde (Intermediate 107) followed by Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14) as a white solid. LC-MS (ESI): mass calced for: C₂₆H₂₅CIFN₅O₃, 509.16; m/z found, 510.2 [M+H]⁺. ¹H NMR (400 MHZ, DMSO-d₆) δ 11.05 (s, 1H), 8.15 (s, 1H), 7.72 (d, J=7.2 Hz, 1H), 7.66 (dd, J=10.4, 5.2 Hz, 2H), 6.80 (d, J=3.6 Hz, 1H), 5.20-5.15 (m, 1H), 4.67 (d, J=17.4 Hz, 1H), 4.49 (d, J=17.4 Hz, 1H), 4.10 (s, 2H), 3.80 (s, 3H), 2.97-2.89 (m, 1H), 2.63 (d, J=17.8 Hz, 5H), 2.46-2.42 (m, 1H), 2.09-2.05 (m, 1H), 1.71 (s, 4H). 1° F. NMR (400 MHZ, DMSO-d₆) δ-121.73 (ppm).

### Example 278:3-(5-(8-(2-methoxyethoxy)-4-(pyrrolidin-1-ylmethyl)-1,5-naphthyridin-2-yl)-1-oxoisoindolin-2-yl) piperidine-2,6-dione

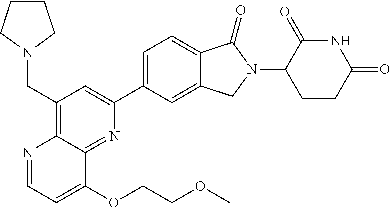 [SMILES: COCCOc1ccnc2c(CN3CCCC3)cc(-c3ccc4c(c3)CN(C3CCC(=O)NC3=O)C4=O)nc12]

The title compound was prepared in a manner analogous to Example 203 by alkylation between pyrrolidine and 4-(bromomethyl)-2-chloro-8-(2-methoxyethoxy)-1,5-naphthyridine (Intermediate 108) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calced for: C₂₉H₃₁N₅O₅, 529.23; m/z found, 530.3 [M+H]⁺. ¹H NMR (400 MHz, DMSO-d₆) δ 11.02 (s, 1H), 8.80 (d, J=5.2 Hz, 1H), 8.44 (d, J=4.2 Hz, 2H), 8.39 (d, J=8.2 Hz, 1H), 8.14 (s, 1H), 7.93 (d, J=8.0 Hz, 1H), 7.33 (d, J=5.2 Hz, 1H), 5.20-5.14 (m, 1H), 4.62 (d, J=17.6 Hz, 1H), 4.52-4.46 (m, 3H), 4.42 (s, 2H), 3.90-3.84 (m, 2H), 3.43 (s, 3H), 2.98-2.93 (m, 1H), 2.74 (s, 4H), 2.63 (d, J=16.4 Hz, 1H), 2.41-2.39 (m, 1H), 2.08-2.02 (m, 1H), 1.81 (s, 4H).

### Example 279:3-(1-oxo-5-(4-(pyrrolidin-1-ylmethyl)-8-((1,1,1-trifluoropropan-2-yl)oxy)-1,5-naphthyridin-2-yl) isoindolin-2-yl) piperidine-2,6-dione

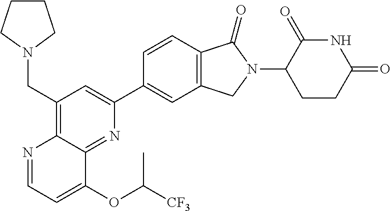 [SMILES: CC(Oc1ccnc2c(CN3CCCC3)cc(-c3ccc4c(c3)CN(C3CCC(=O)NC3=O)C4=O)nc12)C(F)(F)F]

The title compound was prepared in a manner analogous to Example 203 by alkylation between pyrrolidine and 4-(bromomethyl)-2-chloro-8-((1,1,1-trifluoropropan-2-yl)oxy)-1,5-naphthyridine (Intermediate 109) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a pink solid. LC-MS (ESI): mass calced for: C₂₉H₂₈F₃N₅O₄, 567.57; m/z found, 568.3 [M+H]⁺. ¹H NMR (400 MHZ, DMSO-d₆) δ 11.03 (s, 1H), 8.92 (d, J=5.2 Hz, 1H), 8.67 (s, 1H), 8.47 (s, 1H), 8.43 (d, J=8.0 Hz, 1H), 8.14 (s, 1H), 7.96 (d, J=8.0 Hz, 1H), 7.61 (d, J=5.4 Hz, 1H), 5.87-5.75 (m, 1H), 5.20-5.15 (m, 1H), 4.74 (s, 2H), 4.64 (d, J=17.0 Hz, 1H), 4.50 (d, J=17.0 Hz, 1H), 3.08 (s, 4H), 3.00-2.90 (m, 1H), 2.63 (d, J=16.2 Hz, 1H), 2.48-2.40 (m, 1H), 2.10-2.02 (m, 1H), 1.91 (s, 4H), 1.64 (d, J=6.2 Hz, 3H). ¹⁹F NMR (400 MHZ, DMSO-d₆) δ-77.12 (ppm).

### Example 280:3-(5-(8-((1r,3r)-3-methoxycyclobutoxy)-4-(pyrrolidin-1-ylmethyl)-1,5-naphthyridin-2-yl)-1-oxoisoindolin-2-yl) piperidine-2,6-dione

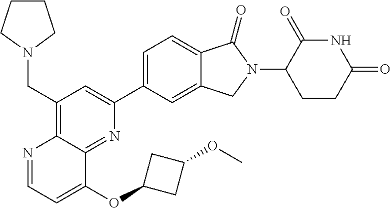 [SMILES: CO[C@H]1C[C@H](Oc2ccnc3c(CN4CCCC4)cc(-c4ccc5c(c4)CN(C4CCC(=O)NC4=O)C5=O)nc23)C1]

The title compound was prepared in a manner analogous to Example 203 by alkylation between pyrrolidine and 4-(bromomethyl)-2-chloro-8-((1r,3r)-3-methoxycyclobutoxy)-1,5-naphthyridine (Intermediate 110) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a yellow solid. LC-MS (ESI): mass calced for: C₃₁H₃₃N₅O₅, 555.1; m/z found, 556.1 [M+H]⁺. ¹H NMR (400 MHZ, DMSO-d₆) δ 11.02 (s, 1H), 8.79 (d, J=5.2 Hz, 1H), 8.47 (s, 2H), 8.42 (d, J=8.2 Hz, 1H), 8.14 (s, 1H), 7.94 (d, J=8.0 Hz, 1H), 7.11 (d, J=5.4 Hz, 1H), 5.21-5.16 (m, 2H), 4.63 (d, J=17.4 Hz, 1H), 4.50 (d, J=17.4 Hz, 3H), 4.17 (s, 1H), 3.22 (s, 3H), 2.93 (d, J=7.0 Hz, 1H), 2.88-2.81 (m, 2H), 2.64 (d, J=16.8 Hz, 5H), 2.55 (d, J=6.8 Hz, 2H), 2.43 (d, J=4.6 Hz, 1H), 2.09-2.04 (m, 1H), 1.85 (s, 4H).

### Example 281:3-(5-(8-((1s,3s)-3-methoxycyclobutoxy)-4-(pyrrolidin-1-ylmethyl)-1,5-naphthyridin-2-yl)-1-oxoisoindolin-2-yl) piperidine-2,6-dione

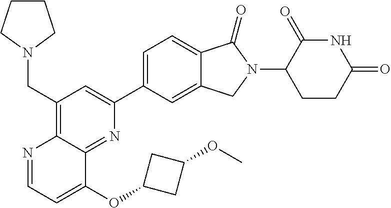 [SMILES: CO[C@H]1C[C@@H](Oc2ccnc3c(CN4CCCC4)cc(-c4ccc5c(c4)CN(C4CCC(=O)NC4=O)C5=O)nc23)C1]

The title compound was prepared in a manner analogous to Example 203 by alkylation between pyrrolidine and of 4-(bromomethyl)-2-chloro-8-((1s,3s)-3-methoxycyclobutoxy)-1,5-naphthyridine (Intermediate 111) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calced for: C₃₁H₃₃N₅O₅, 555.1; m/z found, 556.1 [M+H]⁺. ¹H NMR (400 MHZ, DMSO-d₆) δ 11.03 (s, 1H), 8.78 (d, J=5.2 Hz, 1H), 8.47 (d, J=8.6 Hz, 2H), 8.41 (d, J=7.8 Hz, 1H), 8.14 (s, 1H), 7.93 (d, J=8.0 Hz, 1H), 7.15 (d, J=5.2 Hz, 1H), 5.20-5.15 (m, 1H), 4.80-4.72 (m, 1H), 4.63 (d, J=17.2 Hz, 1H), 4.54-4.35 (m, 3H), 3.75 (t, J=6.6 Hz, 1H), 3.20 (s, 3H), 3.07 (s, 2H), 2.93 (d, J=12.4 Hz, 1H), 2.81-2.63 (m, 5H), 2.45-2.41 (m, 1H), 2.14-2.04 (m, 3H), 1.82 (s, 4H).

### Example 282:3-(4-fluoro-5-(7-((4-fluoropiperidin-1-yl)methyl)-3-(isopropylamino)-1H-pyrazolo[4,3-b]pyridin-5-yl)-1-oxoisoindolin-2-yl) piperidine-2,6-dione

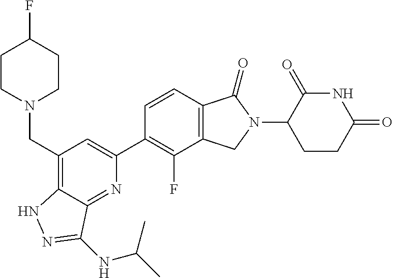 [SMILES: CC(C)Nc1n[nH]c2c(CN3CCC(F)CC3)cc(-c3ccc4c(c3F)CN(C3CCC(=O)NC3=O)C4=O)nc12]

The title compound was prepared in a manner analogous to Example 173 by reductive amination between 4-fluoropiperidine and 5-chloro-3-(isopropylamino)-1-((2-(trimethylsilyl) ethoxy)methyl)-1H-pyrazolo[4,3-b]pyridine-7-carbaldehyde (Intermediate 25 or Intermediate 26) followed by Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14) and de-protection of N-SEM group as a yellow solid. LC-MS (ESI): mass calced for: C₂₈H₃₁F₂N₇O₃, 551.25; m/z found, 552.4 [M+H]⁺. ¹H NMR (400 MHZ, DMSO-d₆) δ 12.05 (s, 1H), 10.97 (s, 1H), 9.82 (s, 1H), 8.16 (t, J=7.4 Hz, 1H), 7.87 (s, 1H), 7.68 (d, J=8.0 Hz, 1H), 5.87 (s, 1H), 5.13-5.08 (m, 1H), 4.92 (d, J=46.8 Hz, 1H), 4.59 (d, J=17.4 Hz, 3H), 4.43 (d, J=17.4 Hz, 1H), 3.99-3.90 (m, 1H), 3.35-3.28 (m, 4H), 2.93-2.83 (m, 1H), 2.56 (d, J=17.0 Hz, 1H), 2.38 (dd, J=13.4, 4.4 Hz, 1H), 2.11-1.71 (m, 5H), 1.20 (d, J=6.4 Hz, 6H). ¹⁹F NMR (400 MHZ, DMSO-d6) δ -73.87,-124.23,-187.32 (ppm).

### Example 283:3-(4-fluoro-5-(3-(isopropylamino)-7-((4-methoxypiperidin-1-yl)methyl)-1H-pyrazolo[4,3-b]pyridin-5-yl)-1-oxoisoindolin-2-yl) piperidine-2,6-dione

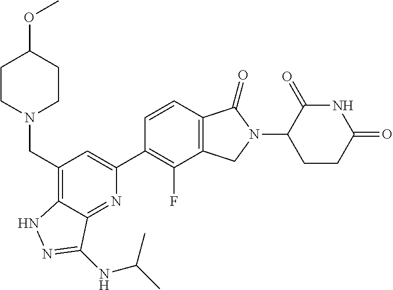 [SMILES: COC1CCN(Cc2cc(-c3ccc4c(c3F)CN(C3CCC(=O)NC3=O)C4=O)nc3c(NC(C)C)n[nH]c23)CC1]

The title compound was prepared in a manner analogous to Example 173 by reductive amination between 4-methoxypiperidine and 5-chloro-3-(isopropylamino)-1-((2-(trimethylsilyl)ethoxy)methyl)-1H-pyrazolo[4,3-b]pyridine-7-carbaldehyde (Intermediate 25 or Intermediate 26) followed by Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14) and de-protection of N-SEM group as a yellow solid. LC-MS (ESI): mass calced for: C₂₉H₃₄FN₇O₄, 563.63; m/z found, 564.4 [M+H]⁺. ¹H NMR (400 MHZ, DMSO-d₆) δ 12.10 (s, 1H), 11.05 (s, 1H), 9.86 (s, 1H), 8.24 (t, J=7.4 Hz, 1H), 7.96 (s, 1H), 7.75 (d, J=8.0 Hz, 1H), 5.96 (s, 1H), 5.20-5.15 (m, 1H), 4.66 (d, J=17.4 Hz, 1H), 4.62 (s, 2H), 4.51 (d, J=17.4 Hz, 1H), 4.07-3.97 (m, 1H), 3.55 (s, 1H), 3.46-3.02 (m, 7H), 3.01-2.90 (m, 1H), 2.64 (d, J=16.8 Hz, 1H), 2.48-2.45 (m, 1H), 2.17 (s, 1H), 2.10-1.97 (m, 2H), 1.80 (s, 1H), 1.54 (s, 1H), 1.28 (d, J=6.4 Hz, 6H). ¹⁹F NMR (400 MHZ, DMSO-d₆) δ-74.54,-124.21 (ppm).

### Example 284:3-(5-(7-((2-oxa-7-azaspiro[3.5]nonan-7-yl)methyl)-3-(isopropylamino)-1H-pyrazolo[4,3-b]pyridin-5-yl)-4-fluoro-1-oxoisoindolin-2-yl) piperidine-2,6-dione

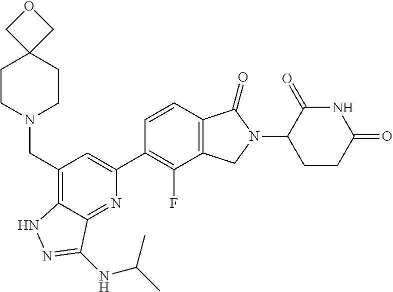 [SMILES: CC(C)Nc1n[nH]c2c(CN3CCC4(CC3)COC4)cc(-c3ccc4c(c3F)CN(C3CCC(=O)NC3=O)C4=O)nc12]

The title compound was prepared in a manner analogous to Example 173 by reductive amination between 2-oxa-7-azaspiro[3.5]nonane and 5-chloro-3-(isopropylamino)-1-((2-(trimethylsilyl)ethoxy)methyl)-1H-pyrazolo[4,3-b]pyridine-7-carbaldehyde (Intermediate 25 or Intermediate 26) followed by Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14) and de-protection of N-SEM group as a yellow solid. LC-MS (ESI): mass calced for: C₃₀H₃₄FN₇O₄, 575.65; m/z found, 576.4 [M+H]⁺. ¹H NMR (400 MHZ, DMSO-d₆) δ 12.10 (s, 1H), 11.05 (s, 1H), 9.94 (s, 1H), 8.24 (t, J=7.4 Hz, 1H), 7.92 (s, 1H), 7.75 (d, J=8.0 Hz, 1H), 6.14 (s, 1H), 5.20-5.15 (m, 1H), 4.66 (d, J=17.4 Hz, 1H), 4.58 (s, 2H), 4.51 (d, J=17.4 Hz, 1H), 4.42 (s, 2H), 4.29 (s, 2H), 4.09-3.96 (m, 1H), 3.43 (s, 2H), 3.00-2.95 (m, 3H), 2.64 (d, J=17.2 Hz, 1H), 2.46 (dd, J=13.8, 4.2 Hz, 1H), 2.24 (s, 2H), 2.10-2.03 (m, 1H), 1.83 (s, 2H), 1.28 (d, J=6.4 Hz, 6H). ¹⁹F NMR (400 MHZ, DMSO-d₆) δ-74.71,-124.20 (ppm).

### Example 285:3-(5-(8-(2-hydroxyethoxy)-4-(pyrrolidin-1-ylmethyl)-1,5-naphthyridin-2-yl)-1-oxoisoindolin-2-yl) piperidine-2,6-dione

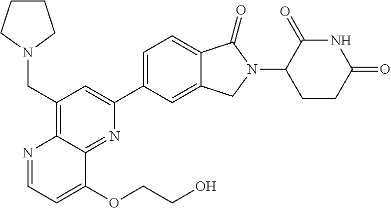 [SMILES: O=C1CCC(N2Cc3cc(-c4cc(CN5CCCC5)c5nccc(OCCO)c5n4)ccc3C2=O)C(=O)N1]

### Step A: 8-(2-((tert-butyldimethylsilyl)oxy) ethoxy)-2-chloro-4-(pyrrolidin-1-ylmethyl)-1,5-naphthyridine

To a solution of 4-(bromomethyl)-8-(2-((tert-butyldimethylsilyl)oxy) ethoxy)-2-chloro-1,5-naphthyridine (Intermediate 112, 180 mg, 417 μmol, 1.0 eq) in DCM (10.00 mL) was added DIPEA (162 mg, 1.25 mmol, 3.0 eq) and the mixture was stirred at room temperature for 0.5 h. Then pyrrolidine (44.5 mg, 625 μmol, 1.5 eq) was added to above mixture and the mixture was stirred at room temperature for 0.5 h. After evaporation, the residue was poured into water (5 mL) and extracted with DCM (10 mL×3). The organic layer was dried over MgSO₄, filtered and concentrated under reduced pressure. The residue was purified by Prep-TLC(DCM/MeOH=10/1 v/v) to obtain 8-(2-((tert-butyldimethylsilyl)oxy) ethoxy)-2-chloro-4-(pyrrolidin-1-ylmethyl)-1,5-naphthyridine (70.0 mg, yield 36%) as a yellow solid. LC-MS (ESI): mass calced for: C₂₁H₃₂ClN₃O₂Si, 421.20; m/z found, 422.2 [M+H]⁺. ¹H NMR (400 MHz, DMSO-d₆) δ 8.82 (t, J=4.8 Hz, 1H), 7.90 (s, 1H), 7.37 (d, J=4.6 Hz, 1H), 4.37-4.33 (m, 2H), 4.16-4.01 (m, 4H), 3.17 (d, J=5.2 Hz, 4H), 1.83 (s, 4H), 0.85 (s, 9H), 0.11 (s, 6H).

### Step B: 3-(5-(8-(2-((tert-butyldimethylsilyl)oxy) ethoxy)-4-(pyrrolidin-1-ylmethyl)-1,5-naphthyridin-2-yl)-1-oxoisoindolin-2-yl) piperidine-2,6-dione

To a solution of 8-(2-((tert-butyldimethylsilyl)oxy) ethoxy)-2-chloro-4-(pyrrolidin-1-ylmethyl)-1,5-naphthyridine (70.0 mg, 166 μmol, 1.0 eq) and 3-(1-oxo-5-(4,4,5,5-tetramethyl-1,3,2-dioxaborolan-2-yl) isoindolin-2-yl) piperidine-2,6-dione (Intermediate 13, 73.7 mg, 199 μmol, 1.2 eq) in dioxane (2 mL) and water (0.2 mL) were added 1,1′-Bis(di-t-butylphosphino) ferrocene palladium dichloride (10.8 mg, 16.6 μmol, 0.1 eq) and Potassium phosphate tribasic (106 mg, 498 μmol, 3.0 eq). The mixture was stirred under N₂ at 95° C. for 2 h. The reaction mixture was cooled to room temperature and concentrated under reduced pressure. The residue was poured into water (5 mL) and extracted with EtOAc (10 mL×3). The organic layer was dried over MgSO₄, filtered and concentrated under reduced pressure. The residue was purified by Prep-TLC(DCM/MeOH=10/1 v/v) to obtain 3-(5-(8-(2-((tert-butyldimethylsilyl)oxy) ethoxy)-4-(pyrrolidin-1-ylmethyl)-1,5-naphthyridin-2-yl)-1-oxoisoindolin-2-yl) piperidine-2,6-dione (30.0 mg, yield 26%) as a yellow solid. LC-MS (ESI): mass calced for: C₃₄H₄₃N₅O₅Si, 629.30; m/z found, 630.2 [M+H]⁺.

### Step C: 3-(5-(8-(2-hydroxyethoxy)-4-(pyrrolidin-1-ylmethyl)-1,5-naphthyridin-2-yl)-1-oxoisoindolin-2-yl) piperidine-2,6-dione

To a solution of 3-(5-(8-(2-((tert-butyldimethylsilyl)oxy) ethoxy)-4-(pyrrolidin-1-ylmethyl)-1,5-naphthyridin-2-yl)-1-oxoisoindolin-2-yl) piperidine-2,6-dione (30.0 mg, 47.6 μmol, 1.0 eq) in DCM (2.00 mL) was added dropwise TFA (0.5 mL) at 25° C. and the mixture was stirred at room temperature for 2 h. After evaporation, the crude product was purified by Prep-HPLC with YMC-Actus Triart 18C(5 μm, 20×250 mm), and mobile phase of 5-99% ACN in water (0.1% FA) over 10 min and then hold at 100% ACN for 2 min, at a flow rate of 25 mL/min to give 3-(5-(8-(2-hydroxyethoxy)-4-(pyrrolidin-1-ylmethyl)-1,5-naphthyridin-2-yl)-1-oxoisoindolin-2-yl) piperidine-2,6-dione formate (4.90 mg, yield 18%) as a yellow solid. LC-MS (ESI): mass calced for: C₂₈H₂₉N₅O₅, 515.22; m/z found, 516.3 [M+H]⁺. ¹H NMR (400 MHZ, DMSO-d₆) δ 11.03 (s, 1H), 8.79 (d, J=5.2 Hz, 1H), 8.41 (dd, J=16.8, 8.2 Hz, 3H), 8.18 (s, 1H), 7.92 (d, J=8.0 Hz, 1H), 7.32 (d, J=5.2 Hz, 1H), 5.20-5.15 (m, 1H), 5.03 (s, 1H), 4.62 (d, J=17.4 Hz, 1H), 4.49 (d, J=17.4 Hz, 1H), 4.39-4.34 (m, 4H), 3.93 (d, J=4.8 Hz, 2H), 2.97-2.89 (m, 1H), 2.66-2.61 (m, 5H), 2.46-2.42 (m, 1H), 2.10-2.02 (m, 1H), 1.79 (s, 4H).

### Example 286:3-(5-(7-((2-oxa-6-azaspiro[3.3]heptan-6-yl)methyl)-3-(isopropylamino)-1H-pyrazolo[4,3-b]pyridin-5-yl)-4-fluoro-1-oxoisoindolin-2-yl) piperidine-2,6-dione

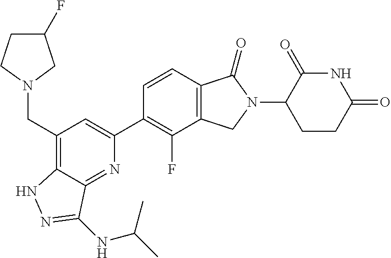 [SMILES: CC(C)Nc1n[nH]c2c(CN3CCC(F)C3)cc(-c3ccc4c(c3F)CN(C3CCC(=O)NC3=O)C4=O)nc12]

The title compound was prepared in a manner analogous to Example 173 by reductive amination between 3-fluoropyrrolidine hydrochloride and 5-chloro-3-(isopropylamino)-1-((2-(trimethylsilyl)ethoxy)methyl)-1H-pyrazolo[4,3-b]pyridine-7-carbaldehyde (Intermediate 25 or Intermediate 26) followed by Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14) and de-protection of N-SEM group as a yellow solid. LC-MS (ESI): mass calced for: C₂₇HF₂N₇O₃, 537.23; m/z found, 538.3 [M+H]. ¹H NMR (400 MHZ, DMSO-d₆) δ 12.11 (s, 1H), 11.04 (s, 1H), 8.21 (t, J=7.4 Hz, 1H), 7.99 (s, 1H), 7.75 (d, J=8.0 Hz, 1H), 5.88 (s, 1H), 5.47 (d, J=52.6 Hz, 1H), 5.20-5.14 (m, 1H), 4.76-4.61 (m, 3H), 4.50 (d, J=17.4 Hz, 1H), 4.03-4.01 (m, 1H), 3.64-3.55 (m, 2H), 3.24 (s, 2H), 2.99-2.90 (m, 1H), 2.66-2.60 (m, 1H), 2.48-2.41 (m, 1H), 2.25 (s, 2H), 2.07-2.03 (m, 1H), 1.27 (d, J=6.4 Hz, 6H). ¹⁹F NMR (400 MHz, DMSO-d₆) δ-74.08,-124.24,-171.75 (ppm).

### Example 287:3-(5-(7-((2-oxa-6-azaspiro[3.3]heptan-6-yl)methyl)-3-(isopropylamino)-1H-pyrazolo[4,3-b]pyridin-5-yl)-4-fluoro-1-oxoisoindolin-2-yl) piperidine-2,6-dione

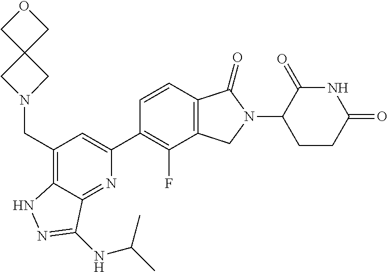 [SMILES: CC(C)Nc1n[nH]c2c(CN3CC4(COC4)C3)cc(-c3ccc4c(c3F)CN(C3CCC(=O)NC3=O)C4=O)nc12]

The title compound was prepared in a manner analogous to Example 173 by reductive amination between 2-oxa-6-azaspiro[3.3]heptane and 5-chloro-3-(isopropylamino)-1-((2-(trimethylsilyl)ethoxy)methyl)-1H-pyrazolo[4,3-b]pyridine-7-carbaldehyde (Intermediate 25 or Intermediate 26) followed by Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14) and de-protection of N-SEM group as a yellow solid. LC-MS (ESI): mass calced for: C₂₈H₃₀FN₇O₄, 547.23; m/z found, 548.3 [M+H]. ¹H NMR (400 MHZ, DMSO-d₆) δ 12.06 (s, 1H), 11.04 (s, 1H), 10.12 (s, 1H), 8.20 (t, J=7.4 Hz, 1H), 7.83 (s, 1H), 7.75 (d, J=8.0 Hz, 1H), 5.92 (s, 1H), 5.20-5.15 (m, 1H), 4.70-4.52 (m, 7H), 4.50 (d, J=17.4 Hz, 1H), 4.42 (s, 2H), 4.36 (s, 2H), 4.07-3.94 (m, 1H), 2.99-2.89 (m, 1H), 2.63 (d, J=15.0 Hz, 1H), 2.48-2.42 (m, 1H), 2.09-2.04 (m, 1H), 1.26 (d, J=6.4 Hz, 6H). ¹⁹F NMR (400 MHZ, DMSO) δ -73.90,-124.32 (ppm).

### Example 288:3-(4-fluoro-5-(3-(isopropylamino)-7-((tetrahydro-1H-furo[3,4-c]pyrrol-5 (3H)-yl)methyl)-1H-pyrazolo[4,3-b]pyridin-5-yl)-1-oxoisoindolin-2-yl) piperidine-2,6-dione

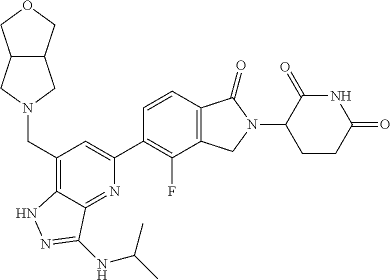 [SMILES: CC(C)Nc1n[nH]c2c(CN3CC4COCC4C3)cc(-c3ccc4c(c3F)CN(C3CCC(=O)NC3=O)C4=O)nc12]

The title compound was prepared in a manner analogous to Example 173 by reductive amination between hexahydro-1H-furo[3,4-c]pyrrole and 5-chloro-3-(isopropylamino)-1-((2-(trimethylsilyl)ethoxy)methyl)-1H-pyrazolo[4,3-b]pyridine-7-carbaldehyde (Intermediate 25 or Intermediate 26) followed by Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14) and de-protection of N-SEM group as a yellow solid. LC-MS (ESI): mass calced for: C₂₉H₃₂FN₇O₄, 561.25; m/z found, 562.4 [M+H]⁺. ¹H NMR (400 MHZ, DMSO-d₆) δ 12.08 (s, 1H), 11.04 (s, 1H), 10.45 (s, 1H), 8.22 (t, J=7.4 Hz, 1H), 7.97 (s, 1H), 7.75 (d, J=8.0 Hz, 1H), 5.85 (s, 1H), 5.20-5.15 (m, 1H), 4.66 (d, J=17.4 Hz, 3H), 4.50 (d, J=17.4 Hz, 1H), 4.08-3.95 (m, 1H), 3.78 (d, J=7.2 Hz, 4H), 3.47 (s, 2H), 3.09-2.84 (m, 4H), 2.63 (d, J=16.8 Hz, 1H), 2.54 (d, J=4.4 Hz, 1H), 2.46 (dd, J=13.2, 4.4 Hz, 1H), 2.11-2.02 (m, 1H), 1.27 (d, J=6.4 Hz, 6H). ¹⁹F NMR (400 MHZ, DMSO-d₆) δ-74.50,-124.18 (ppm).

### Example 289:3-(5-(7-((7-oxa-2-azaspiro[3.5]nonan-2-yl)methyl)-3-(isopropylamino)-1H-pyrazolo[4,3-b]pyridin-5-yl)-4-fluoro-1-oxoisoindolin-2-yl) piperidine-2,6-dione

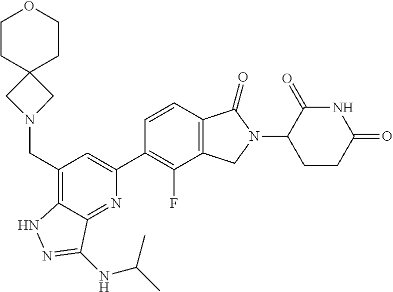 [SMILES: CC(C)Nc1n[nH]c2c(CN3CC4(CCOCC4)C3)cc(-c3ccc4c(c3F)CN(C3CCC(=O)NC3=O)C4=O)nc12]

The title compound was prepared in a manner analogous to Example 173 by reductive amination between 7-oxa-2-azaspiro[3.5]nonane and 5-chloro-3-(isopropylamino)-1-((2-(trimethylsilyl)ethoxy)methyl)-1H-pyrazolo[4,3-b]pyridine-7-carbaldehyde (Intermediate 25 or Intermediate 26) followed by Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14) and de-protection of N-SEM group as a yellow solid. LC-MS (ESI): mass calced for: C₃₀H₃₄FN₇O₄, 575.26; m/z found, 576.0 [M+H]. ¹H NMR (400 MHZ, DMSO-d₆) δ 12.12 (s, 1H), 11.04 (s, 1H), 10.36 (s, 1H), 8.18 (t, J=7.4 Hz, 1H), 7.92 (s, 1H), 7.75 (d, J=8.0 Hz, 1H), 5.20-5.15 (m, 1H), 4.74 (s, 2H), 4.66 (d, J=17.4 Hz, 1H), 4.50 (d, J=17.4 Hz, 1H), 4.19-3.96 (m, 5H), 3.47 (s, 4H), 3.00-2.89 (m, 1H), 2.63 (d, J=18.6 Hz, 1H), 2.45 (dd, J=13.6, 4.4 Hz, 1H), 2.10-2.02 (m, 1H), 1.80 (s, 4H), 1.26 (d, J=6.4 Hz, 6H). ¹⁹F NMR (400 MHz, DMSO-d₆) δ-74.28,-124.23 (ppm).

### Example 290:3-(4-fluoro-5-(3-(isopropylamino)-7-((4-(trifluoromethyl) piperidin-1-yl)methyl)-2H-pyrazolo[4,3-b]pyridin-5-yl)-1-oxoisoindolin-2-yl) piperidine-2,6-dione

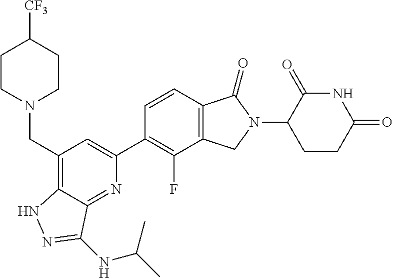 [SMILES: CC(C)Nc1n[nH]c2c(CN3CCC(C(F)(F)F)CC3)cc(-c3ccc4c(c3F)CN(C3CCC(=O)NC3=O)C4=O)nc12]

The title compound was prepared in a manner analogous to Example 173 by reductive amination between 4-(trifluoromethyl) piperidine and 5-chloro-3-(isopropylamino)-1-((2-(trimethylsilyl)ethoxy)methyl)-1H-pyrazolo[4,3-b]pyridine-7-carbaldehyde (Intermediate 25 or Intermediate 26) followed by Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14) and de-protection of N-SEM group as a yellow solid. LC-MS (ESI): mass calced for: C₂₉H₃₁F₄N₇O₃, 601.2; m/z found, 602.2 [M+H]⁺. ¹H NMR (400 MHZ, DMSO-d₆) δ 12.16 (s, 1H), 11.04 (s, 1H), 10.09 (s, 1H), 8.23 (t, J=7.4 Hz, 1H), 7.94 (s, 1H), 7.75 (d, J=8.0 Hz, 1H), 5.20-5.15 (m, 1H), 4.64 (t, J=17.4 Hz, 3H), 4.50 (d, J=17.4 Hz, 1H), 4.03-3.99 (m, 1H), 3.60 (s, 2H), 3.18-2.98 (m, 2H), 2.96-2.89 (m, 1H), 2.63 (d, J=16.8 Hz, 2H), 2.45 (dd, J=13.2, 4.4 Hz, 1H), 2.05 (dd, J=11.0, 5.8 Hz, 3H), 1.74 (d, J=15.4 Hz, 2H), 1.27 (d, J=6.4 Hz, 6H). ¹⁹F NMR (400 MHZ, DMSO-d₆) δ-74.23,-124.21 (ppm).

### Example 291:3-(5-(3-(ethylamino)-7-(pyrrolidin-1-ylmethyl)-1H-pyrazolo[4,3-b]pyridin-5-yl)-4-fluoro-1-oxoisoindolin-2-yl) piperidine-2,6-dione

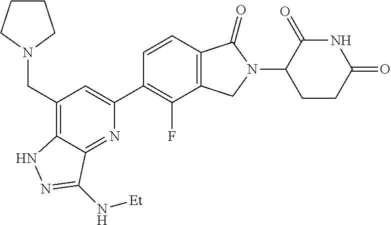 [SMILES: CCNc1n[nH]c2c(CN3CCCC3)cc(-c3ccc4c(c3F)CN(C3CCC(=O)NC3=O)C4=O)nc12]

The title compound was prepared in a manner analogous to Example 173 by reductive amination between pyrrolidine and 5-chloro-3-(ethylamino)-2-((2-(trimethylsilyl)ethoxy)methyl)-2H-pyrazolo[4,3-b]pyridine-7-carbaldehyde (Intermediate 113) followed by Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14) followed by de-protection of N-SEM group as a yellow solid. LC-MS (ESI): mass calced for: C₂₆H₂₈FN703, 505.3; m/z found, 506.1 [M+H]⁺. ¹H NMR (400 MHZ, DMSO-d₆) δ 12.16 (s, 1H), 11.05 (s, 1H), 10.09 (s, 1H), 8.19 (t, J=7.4 Hz, 1H), 7.97 (s, 1H), 7.76 (d, J=8.0 Hz, 1H), 5.20-5.15 (m, 1H), 4.69 (s, 2H), 4.66 (d, J=17.4 Hz, 1H), 4.51 (d, J=17.4 Hz, 1H), 3.54 (s, 2H), 3.20 (d, J=21.8 Hz, 4H), 2.97-2.89 (m, 1H), 2.64 (d, J=16.8 Hz, 1H), 2.48-2.42 (m, 1H), 2.08-1.99 (m, 3H), 1.90 (s, 2H), 1.25 (t, J=7.2 Hz, 3H). ¹⁹F NMR (400 MHZ, DMSO-d₆) δ-74.04,-124.31 (ppm).

### Example 292:3-(5-(1-(1,1-dioxidothietan-3-yl)-4-(pyrrolidin-1-ylmethyl)-1H-pyrrolo[2,3-b]pyridin-6-yl)-3-methyl-1-oxoisoindolin-2-yl) piperidine-2,6-dione

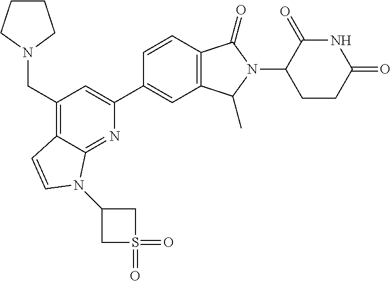 [SMILES: CC1c2cc(-c3cc(CN4CCCC4)c4ccn(C5CS(=O)(=O)C5)c4n3)ccc2C(=O)N1C1CCC(=O)NC1=O]

The title compound was prepared in a manner analogous to Example 266 by reductive amination between pyrrolidine and 6-chloro-1H-pyrrolo[2,3-b]pyridine-4-carbaldehyde (Intermediate 9) followed by displacement with 3-bromothietane 1,1-dioxide and Suzuki coupling with 3-(3-methyl-1-oxo-5-(4,4,5,5-tetramethyl-1,3,2-dioxaborolan-2-yl) isoindolin-2-yl) piperidine-2,6-dione (Intermediate 28) as a white solid. LC-MS (ESI): mass calced for: C₂₉H₃₁N₅O₅S, 561.20; m/z found, 562.2 [M+H]⁺. ¹H NMR (400 MHZ, DMSO-d₆) δ 10.95 (d, J=12.6 Hz, 1H), 8.52 (d, J=3.6 Hz, 1H), 8.34 (d, J=8.0 Hz, 1H), 8.16 (s, 1H), 7.87 (s, 1H), 7.79-7.74 (m, 2H), 6.75 (d, J=3.6 Hz, 1H), 5.84-5.74 (m, 1H), 5.03 (td, J=13.6, 6.2 Hz, 2H), 4.92-4.73 (m, 4H), 3.98 (s, 2H), 2.91-2.77 (m, 1H), 2.76-2.60 (m, 2H), 2.56 (s, 4H), 2.08-1.99 (m, 1H), 1.74 (s, 4H), 1.57-1.53 (m, 3H).

### Example 293:3-(5-(3-(isopropylamino)-7-(pyrrolidin-1-ylmethyl)-1H-pyrazolo[4,3-b]pyridin-5-yl)-3-methyl-1-oxoisoindolin-2-vl) piperidine-2,6-dione

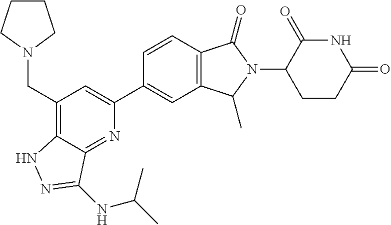 [SMILES: CC(C)Nc1n[nH]c2c(CN3CCCC3)cc(-c3ccc4c(c3)C(C)N(C3CCC(=O)NC3=O)C4=O)nc12]

The title compound was prepared in a manner analogous to Example 173 by reductive amination between pyrrolidine and 5-chloro-3-(isopropylamino)-1-((2-(trimethylsilyl)ethoxy)methyl)-1H-pyrazolo[4,3-b]pyridine-7-carbaldehyde (Intermediate 25) followed by Suzuki coupling with 3-(3-methyl-1-oxo-5-(4,4,5,5-tetramethyl-1,3,2-dioxaborolan-2-yl) isoindolin-2-yl) piperidine-2,6-dione (Intermediate 28) followed by de-protection of N-SEM group as a yellow solid. LC-MS (ESI): mass calced for: C₂₈H₃₃N₇O₃, 515.26; m/z found, 516.2 [M+H]⁺. ¹H NMR (400 MHZ, DMSO-d₆) δ 12.14 (s, 1H), 10.96 (d, J=13.6 Hz, 1H), 10.23 (s, 1H), 8.37 (s, 1H), 8.32 (d, J=8.2 Hz, 1H), 8.21 (s, 1H), 7.86-7.78 (m, 1H), 6.02 (s, 1H), 4.92-4.74 (m, 2H), 4.66 (s, 2H), 4.07-3.96 (m, 1H), 3.55 (s, 2H), 3.26 (s, 2H), 2.93-2.55 (m, 3H), 2.18-1.99 (m, 3H), 1.91 (s, 2H), 1.54 (t, J=7.6 Hz, 3H), 1.29 (d, J=6.4 Hz, 6H). ¹⁹F NMR (400 MHZ, DMSO-d₆) δ-74.55 (ppm).

### Example 294:3-(5-(3-(isopropylamino)-7-(pyrrolidin-1-ylmethyl)-1H-pyrazolo[4,3-b]pyridin-5-yl)-1-oxoisoindolin-2-yl)-3-methylpiperidine-2,6-dione

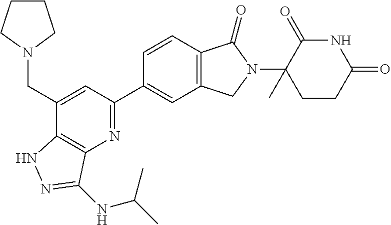 [SMILES: CC(C)Nc1n[nH]c2c(CN3CCCC3)cc(-c3ccc4c(c3)CN(C3(C)CCC(=O)NC3=O)C4=O)nc12]

The title compound was prepared in a manner analogous to Example 173 by Suzuki coupling of 5-chloro-N-isopropyl-7-(pyrrolidin-1-ylmethyl)-1-((2-(trimethylsilyl)ethoxy)methyl)-1H-pyrazolo[4,3-b]pyridin-3-amine (Intermediate 25) with 3-methyl-3-(1-oxo-5-(4,4,5,5-tetramethyl-1,3,2-dioxaborolan-2-yl) isoindolin-2-yl) piperidine-2,6-dione (Intermediate 114) followed by de-protection of N-SEM group as a yellow solid. LC-MS (ESI): mass calced for: C₂₈H₃₃N₇O₃, 515.26; m/z found, 516.2 [M+H]⁺. ¹H NMR (400 MHz, DMSO-d₆) δ 11.78 (s, 1H), 10.89 (s, 1H), 8.35 (s, 1H), 8.29 (d, J=8.0 Hz, 1H), 8.14 (s, 1H), 7.92 (s, 1H), 7.74 (d, J=8.0 Hz, 1H), 5.60 (d, J=7.4 Hz, 1H), 4.76 (q, J=17.2 Hz, 2H), 4.01 (dd, J=13.6, 6.4 Hz, 3H), 2.81-2.66 (m, 5H), 2.56 (s, 2H), 1.94 (d, J=11.6 Hz, 1H), 1.78 (s, 4H), 1.71 (s, 3H), 1.28 (d, J=6.4 Hz, 6H).

### Example 295:1.3-(5-(3-(cyclobutylamino)-7-(pyrrolidin-1-ylmethyl)-1H-pyrazolo[4,3-b]pyridin-5-yl)-4-fluoro-1-oxoisoindolin-2-yl) piperidine-2,6-dione

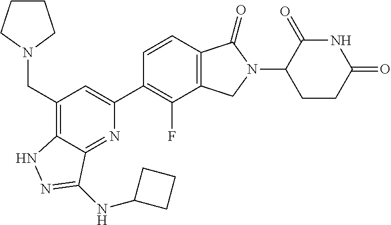 [SMILES: O=C1CCC(N2Cc3c(ccc(-c4cc(CN5CCCC5)c5[nH]nc(NC6CCC6)c5n4)c3F)C2=O)C(=O)N1]

The title compound was prepared in a manner analogous to Example 173 by reductive amination between pyrrolidine and 5-chloro-3-(cyclobutylamino)-1-((2-(trimethylsilyl)ethoxy)methyl)-1H-pyrazolo[4,3-b]pyridine-7-carbaldehyde (Intermediate 115) followed by Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14) and de-protection of N-SEM group as a yellow solid. LC-MS (ESI): mass calced for: C₂₈H₃₀FN703, 531.24; m/z found, 532.3 [M+H]. ¹H NMR (400 MHZ, DMSO-d₆) δ 12.17 (s, 1H), 11.04 (s, 1H), 10.05 (s, 1H), 8.20 (t, J=7.4 Hz, 1H), 7.96 (s, 1H), 7.76 (d, J=8.0 Hz, 1H), 6.48 (s, 1H), 5.20-5.15 (m, 1H), 4.68 (s, 2H), 4.66 (d, J=17.4 Hz, 1H), 4.50 (d, J=17.4 Hz, 1H), 4.37-4.26 (m, 1H), 3.52 (s, 2H), 3.21 (s, 2H), 3.00-2.87 (m, 1H), 2.63 (d, J=17.0 Hz, 1H), 2.44 (s, 1H), 2.34-2.28 (m, 2H), 2.10-2.05 (m, 5H), 1.88 (s, 2H), 1.72-1.67 (m, 2H). ¹⁹F NMR (400 MHZ, DMSO-d₆) δ-73.81,-124.29 (ppm).

### Example 296:3-(4-fluoro-5-(3-(methylamino)-7-(pyrrolidin-1-ylmethyl)-1H-pyrazolo[4,3-b]pyridin-5-yl)-1-oxoisoindolin-2-yl) piperidine-2,6-dione

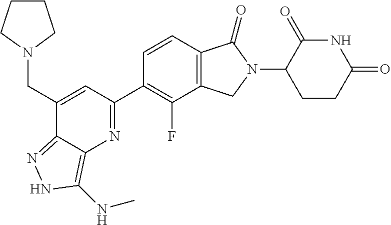 [SMILES: CNc1[nH]nc2c(CN3CCCC3)cc(-c3ccc4c(c3F)CN(C3CCC(=O)NC3=O)C4=O)nc12]

The title compound was prepared in a manner analogous to Example 173 by reductive amination between pyrrolidine and 5-chloro-3-(methylamino)-2-((2-(trimethylsilyl)ethoxy)methyl)-2H-pyrazolo[4,3-b]pyridine-7-carbaldehyde (Intermediate 116) followed by Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14) followed by de-protection of N-SEM group as a yellow solid. LC-MS (ESI): mass calced for: C₂₅H₂₆FN703, 491.21; m/z found, 492.2 [M+H]⁺. ¹H NMR (400 MHZ, DMSO-d₆) δ 12.15 (s, 1H), 11.05 (s, 1H), 10.01 (s, 1H), 8.17 (t, J=7.4 Hz, 1H), 7.97 (s, 1H), 7.77 (d, J=8.0 Hz, 1H), 5.20-5.15 (m, 1H), 4.67 (d, J=17.4 Hz, 3H), 4.51 (d, J=17.4 Hz, 1H), 3.52 (s, 2H), 3.23 (s, 2H), 3.00-2.88 (m, 4H), 2.63 (d, J=18.2 Hz, 1H), 2.45 (d, J=5.0 Hz, 1H), 2.09-2.05 (m, 3H), 1.89 (s, 2H). ¹⁹F NMR (400 MHZ, DMSO-d₆) δ-74.19,-124.35 (ppm).

### Example 297: N-(5-(2-(2,6-dioxopiperidin-3-yl)-4-fluoro-1-oxoisoindolin-5-yl)-7-(pyrrolidin-1-ylmethyl)-1H-pyrazolo[4,3-b]pyridin-3-yl) methanesulfonamide

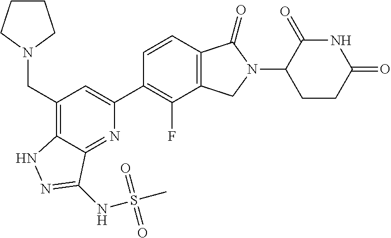 [SMILES: CS(=O)(=O)Nc1n[nH]c2c(CN3CCCC3)cc(-c3ccc4c(c3F)CN(C3CCC(=O)NC3=O)C4=O)nc12]

### Step A: N-(5-(2-(2,6-dioxopiperidin-3-yl)-4-fluoro-1-oxoisoindolin-5-yl)-7-(pyrrolidin-1-ylmethyl)-1-((2-(trimethylsilyl)ethoxy)methyl)-1H-pyrazolo[4,3-b]pyridin-3-yl) methanesulfonamide

To a solution of N-(5-chloro-7-(pyrrolidin-1-ylmethyl)-1-((2-(trimethylsilyl)ethoxy)methyl)-1H-pyrazolo[4,3-b]pyridin-3-yl) methanesulfonamide (Intermediate 117, 110 mg, 239 μmol, 1.0 eq), 3-(4-fluoro-1-oxo-5-(4,4,5,5-tetramethyl-1,3,2-dioxaborolan-2-yl) isoindolin-2-yl) piperidine-2,6-dione (Intermediate 14, 139 mg, 359 μmol, 1.5 eq), and Potassium phosphate, tribasic (152 mg, 717 μmol, 3.0 eq) in 1,4-Dioxane (4.00 mL) and water (0.40 mL) was added 1,1′-Bis(di-t-butylphosphino) ferrocene palladium dichloride (15.6 mg, 23.9 μmol, 0.1 eq) and the mixture was stirred under N₂ at 95° C. for 1 h. After cooled to room temperature, the solution was filtered and the filtrate was concentrated in vacuum. The mixture was purified by Prep-TLC(DCM/MeOH=20/1 v/v) to give N-(5-(2-(2,6-dioxopiperidin-3-yl)-4-fluoro-1-oxoisoindolin-5-yl)-7-(pyrrolidin-1-ylmethyl)-1-((2-(trimethylsilyl)ethoxy)methyl)-1H-pyrazolo[4,3-b]pyridin-3-yl) methanesulfonamide (35.0 mg, yield 21%) as a brown oil. LC-MS (ESI): mass calced for: C₃₁H₄₀FN706SSi, 685.25; m/z found, 686.3 [M+H]⁺.

### Step B: N-(5-(2-(2,6-dioxopiperidin-3-yl)-4-fluoro-1-oxoisoindolin-5-yl)-7-(pyrrolidin-1-ylmethyl)-1H-pyrazolo[4,3-b]pyridin-3-yl) methane sulfonamide

To the solution of N-(5-(2-(2,6-dioxopiperidin-3-yl)-4-fluoro-1-oxoisoindolin-5-yl)-7-(pyrrolidin-1-ylmethyl)-1-((2-(trimethylsilyl)ethoxy)methyl)-1H-pyrazolo[4,3-b]pyridin-3-yl) methanesulfonamide (40.0 mg, 58.3 μmol, 1.0 eq) in DCM (5.00 mL) was added TFA (1.50 mL) at 0° C. The mixture was stirred at 27° C. for 5 h. After evaporation, the crude product was purified by Prep-HPLC with YMC TA C18 (250×21.2 mm, 5 μm), and mobile phase of 5-99% ACN in water (0.1% TFA) over 10 min and then hold at 100% ACN for 2 min, at a flow rate of 25 mL/min to give N-(5-(2-(2,6-dioxopiperidin-3-yl)-4-fluoro-1-oxoisoindolin-5-yl)-7-(pyrrolidin-1-ylmethyl)-1H-pyrazolo[4,3-b]pyridin-3-yl) methanesulfonamide trifluoroacetate (10.4 mg, yield 32%) as a white solid. LC-MS (ESI): mass calced for: C₂₅H₂₆FN705S, 555.17; m/z found, 556.2 [M+H]⁺. ¹H NMR (400 MHZ, DMSO-d₆) δ 13.55 (s, 1H), 11.04 (s, 1H), 10.52 (s, 1H), 10.13 (s, 1H), 8.20-8.02 (m, 2H), 7.79 (d, J=7.8 Hz, 1H), 5.20-5.15 (m, 1H), 4.78 (s, 2H), 4.67 (d, J=17.6 Hz, 1H), 4.51 (d, J=17.6 Hz, 1H), 3.55 (s, 2H), 3.43 (s, 3H), 3.26 (s, 2H), 3.00-2.88 (m, 1H), 2.63 (d, J=17.2 Hz, 1H), 2.46-2.41 (m, 1H), 2.08 (s, 3H), 1.89 (s, 2H). ¹⁹F NMR (400 MHZ, DMSO-d₆) δ-73.64,-124.30 (ppm).

### Example 298:1-(5-(3-(isopropylamino)-7-(pyrrolidin-1-ylmethyl)-1H-pyrazolo[4,3-b]pyridin-5-yl)-1-oxoisoindolin-2-yl)-3-azabicyclo[3.1.1]heptane-2,4-dione

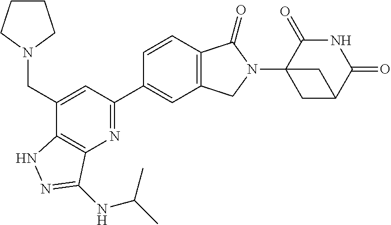 [SMILES: CC(C)Nc1n[nH]c2c(CN3CCCC3)cc(-c3ccc4c(c3)CN(C35CC(C3)C(=O)NC5=O)C4=O)nc12]

The title compound was prepared in a manner analogous to Example 173 by reductive amination between pyrrolidine and 5-chloro-3-(iso-propylamino)-2-((2-(trimethylsilyl)ethoxy)methyl)-2H-pyrazolo[4,3-b]pyridine-7-carbaldehyde (Intermediate 25) followed by Suzuki coupling with 1-(1-oxo-5-(4,4,5,5-tetramethyl-1,3,2-dioxaborolan-2-yl) isoindolin-2-yl)-3-azabicyclo[3.1.1]heptane-2,4-dione (Intermediate 118) followed by de-protection of N-SEM group as a yellow solid. LC-MS (ESI): mass calced for: C₂₈H₃₁N₇O₃, 513.6; m/z found, 514.3 [M+H]⁺. ¹H NMR (400 MHz, DMSO-d₆) δ 12.15 (s, 1H), 11.04 (s, 1H), 10.18 (s, 1H), 8.42 (s, 1H), 8.38 (d, J=8.2 Hz, 1H), 8.25 (s, 1H), 7.89 (d, J=8.0 Hz, 1H), 5.94 (s, 1H), 4.72 (s, 2H), 4.56 (s, 2H), 4.10-4.05 (m, 1H), 3.59 (s, 2H), 3.31 (s, 2H), 3.16 (s, 1H), 2.94 (d, J=2.4 Hz, 4H), 2.15 (s, 2H), 1.97 (s, 2H), 1.34 (d, J=6.4 Hz, 6H). ¹⁹F NMR (400 MHZ, DMSO-d₆) δ-73.77 (ppm).

### Example 299:3-(5-(1-isopropyl-4-(pyrrolidin-1-ylmethyl)-1H-pyrrolo[2,3-b]pyridin-6-yl)-1-oxoisoindolin-2-yl) piperidine-2,6-dione

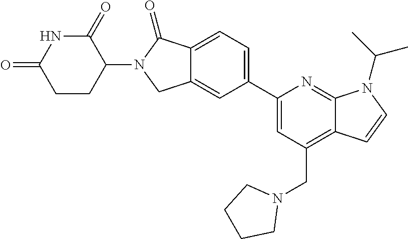 [SMILES: CC(C)n1ccc2c(CN3CCCC3)cc(-c3ccc4c(c3)CN(C3CCC(=O)NC3=O)C4=O)nc21]

The title compound was prepared in a manner analogous to Example 1 by reductive amination between pyrrolidine and 6-chloro-1-isopropyl-1H-pyrrolo[2,3-b]pyridine-4-carbaldehyde (Intermediate 120) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₈H₃₁N₅O₃, 485.6; m/z found, 486.5 [M+H]⁺. ¹H NMR (400 MHZ, DMSO) δ 11.03 (s, 1H), 8.39 (s, 1H), 8.35 (d, J=8.4 Hz, 1H), 7.98 (s, 1H), 7.86 (d, J=8.0 Hz, 1H), 7.77 (d, J=3.6 Hz, 1H), 6.74 (d, J=3.5 Hz, 1H), 5.28-5.21 (m, 1H), 5.17 (dd, J=13.3, 5.1 Hz, 1H), 4.58 (d, J=17.3 Hz, 1H), 4.45 (d, J=17.3 Hz, 1H), 4.28 (s, 2H), 3.00-2.92 (m, 1H), 2.88 (s, 4H), 2.63 (d, J=17.2 Hz, 1H), 2.48-2.38 (m, 1H), 2.08-2.02 (m, 1H), 1.85 (s, 4H), 1.54 (d, J=6.7 Hz, 6H).

### Example 300:3-(5-(1-methyl-4-((4-(methylsulfonyl) piperidin-1-yl)methyl)-1H-pyrrolo[2,3-b]pyridin-6-yl)-1-oxoisoindolin-2-yl) piperidine-2,6-dione

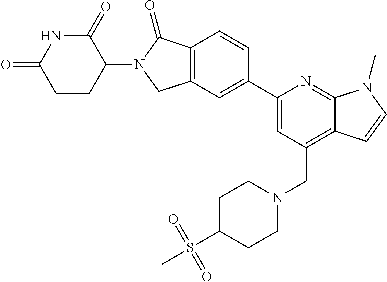 [SMILES: Cn1ccc2c(CN3CCC(S(C)(=O)=O)CC3)cc(-c3ccc4c(c3)CN(C3CCC(=O)NC3=O)C4=O)nc21]

The title compound was prepared in a manner analogous to Example 1 by reductive amination between 4-(methylsulfonyl) piperidine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₈H₃₁N₅O₅S, 549.6; m/z found, 550.1 [M+H]⁺. ¹H NMR (400 MHZ, DMSO) § 11.02 (s, 1H), 8.45-8.26 (m, 2H), 7.84 (d, J=8.0 Hz, 1H), 7.74 (s, 1H), 7.57 (d, J=3.4 Hz, 1H), 6.68 (d, J=3.4 Hz, 1H), 5.16 (dd, J=13.3, 5.1 Hz, 1H), 4.58 (d, J=17.3 Hz, 1H), 4.44 (d, J=17.3 Hz, 1H), 3.89 (d, J=15.0 Hz, 5H), 3.07 (ddd, J=23.6, 10.7, 7.4 Hz, 3H), 2.98-2.87 (m, 4H), 2.63 (dd, J=15.0, 4.3 Hz, 1H), 2.49-2.37 (m, 1H), 2.12-1.94 (m, 5H), 1.67 (dt, J=21.3, 10.7 Hz, 2H).

### Example 301:3-(5-(4-((4-fluoropiperidin-1-yl)methyl)-1-methyl-1H-pyrrolo[2,3-b]pyridin-6-yl)-1-oxoisoindolin-2-yl) piperidine-2,6-dione

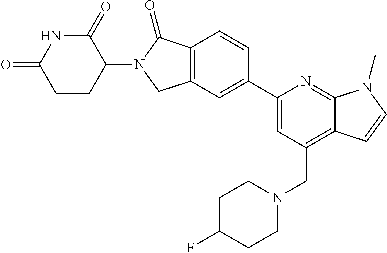 [SMILES: Cn1ccc2c(CN3CCC(F)CC3)cc(-c3ccc4c(c3)CN(C3CCC(=O)NC3=O)C4=O)nc21]

The title compound was prepared in a manner analogous to Example 1 by reductive amination between 4-fluoro-piperidine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₇H₂₈N₅O₃F, 489.5; m/z found, 490.1 [M+H]⁺. ¹H NMR (400 MHZ, DMSO) δ 11.02 (s, 1H), 8.38 (s, 1H), 8.34 (d, J=7.9 Hz, 1H), 7.85 (d, J=8.0 Hz, 1H), 7.75 (s, 1H), 7.56 (s, 1H), 6.68 (s, 1H), 5.16 (dd, J=13.3, 5.1 Hz, 1H), 4.70 (dd, J=45.5, 10.0 Hz, 1H), 4.57 (d, J=17.3 Hz, 1H), 4.44 (d, J=17.2 Hz, 1H), 3.91 (s, 3H), 3.85 (s, 2H), 2.97-2.89 (m, 1H), 2.68-2.57 (m, 3H), 2.48-2.33 (m, 3H), 2.06 (dd, J=10.6, 5.4 Hz, 1H), 1.94-1.70 (m, 4H).

### Example 302:3-(5-(4-(azepan-1-ylmethyl)-1-methyl-1H-pyrrolo[2,3-b]pyridin-6-yl)-1-oxoisoindolin-2-yl) piperidine-2,6-dione

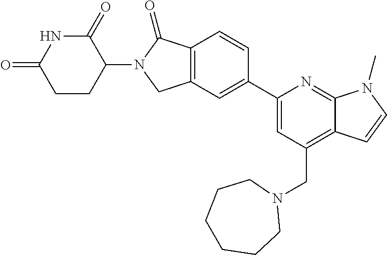 [SMILES: Cn1ccc2c(CN3CCCCCC3)cc(-c3ccc4c(c3)CN(C3CCC(=O)NC3=O)C4=O)nc21]

The title compound was prepared in a manner analogous to Example 1 by reductive amination between azepane and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₈H₃₁N₅O₃, 485.6; m/z found, 486.4 [M+H]⁺. ¹H NMR (400 MHZ, DMSO) § 11.01 (s, 1H), 8.36 (s, 1H), 8.32 (d, J=8.1 Hz, 1H), 8.14 (s, 1H), 7.85 (d, J=8.0 Hz, 1H), 7.78 (s, 1H), 7.56 (d, J=3.4 Hz, 1H), 6.68 (d, J=3.4 Hz, 1H), 5.16 (dd, J=13.2, 5.1 Hz, 1H), 4.58 (d, J=17.2 Hz, 1H), 4.44 (d, J=17.3 Hz, 1H), 4.00 (s, 2H), 3.90 (s, 3H), 3.00-2.88 (m, 1H), 2.69 (s, 4H), 2.66-2.59 (m, 1H), 2.47-2.37 (m, 1H), 2.06 (dd, J=13.0, 7.8 Hz, 1H), 1.60 (s, 9H).

### Example 303:3-(5-(4-((3-hydroxypyrrolidin-1-yl)methyl)-1-methyl-1H-pyrrolo[2,3-b]pyridin-6-yl)-1-oxoisoindolin-2-yl) piperidine-2,6-dione

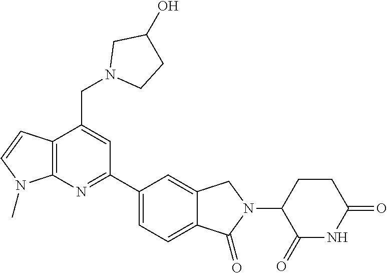 [SMILES: Cn1ccc2c(CN3CCC(O)C3)cc(-c3ccc4c(c3)CN(C3CCC(=O)NC3=O)C4=O)nc21]

The title compound was prepared in a manner analogous to Example 1 by reductive amination between pyrrolidin-3-ol and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₆H₂₇N₅O₄, 473.5; m/z found, 474.4 [M+H]⁺. ¹H NMR (400 MHZ, DMSO) § 11.02 (s, 1H), 8.38 (s, 1H), 8.34 (d, J=8.2 Hz, 1H), 7.85 (d, J=8.0 Hz, 1H), 7.79 (s, 1H), 7.58 (s, 1H), 6.66 (s, 1H), 5.16 (dd, J=13.2, 5.0 Hz, 1H), 4.74 (s, 1H), 4.60-4.41 (m, 2H), 4.25 (s, 1H), 3.92 (s, 1H), 3.91 (s, 3H), 2.99-2.55 (m, 5H), 2.49-2.35 (m, 3H), 2.05 (dd, J=10.8, 5.4 Hz, 2H), 1.61 (s, 1H).

### Example 304:3-(5-(4-((3-fluoropyrrolidin-1-yl)methyl)-1-methyl-1H-pyrrolo[2,3-b]pyridin-6-yl)-1-oxoisoindolin-2-yl) piperidine-2,6-dione

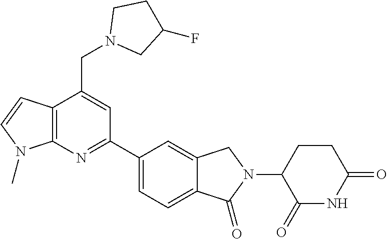 [SMILES: Cn1ccc2c(CN3CCC(F)C3)cc(-c3ccc4c(c3)CN(C3CCC(=O)NC3=O)C4=O)nc21]

The title compound was prepared in a manner analogous to Example 1 by reductive amination between 3-fluoropyrrolidine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₆H₂₆FN₅O₃, 475.5; m/z found, 476.4 [M+H]⁺.

¹H NMR (400 MHZ, DMSO) δ 11.01 (s, 1H), 8.37 (s, 1H), 8.33 (d, J=8.1 Hz, 1H), 7.84 (d, J=8.0 Hz, 1H), 7.75 (s, 1H), 7.56 (d, J=3.4 Hz, 1H), 6.65 (d, J=3.4 Hz, 1H), 5.32-5.10 (m, 2H), 4.51 (dd, J=52.6, 17.3 Hz, 2H), 4.01 (d, J=14.2 Hz, 2H), 3.91 (s, 3H), 2.98-2.82 (m, 3H), 2.78-2.58 (m, 2H), 2.47-2.36 (m, 2H), 2.25-2.08 (m, 1H), 2.05 (dd, J=8.9, 3.7 Hz, 1H), 2.00-1.82 (m, 1H). 1° F. NMR (376 MHz, DMSO) δ -166.54 (s).

### Example 305:3-(5-(4-((4-hydroxypiperidin-1-yl)methyl)-1-methyl-1H-pyrrolo[2,3-b]pyridin-6-yl)-1-oxoisoindolin-2-yl) piperidine-2,6-dione

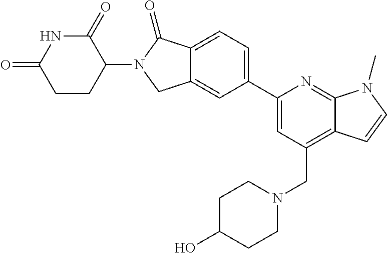 [SMILES: Cn1ccc2c(CN3CCC(O)CC3)cc(-c3ccc4c(c3)CN(C3CCC(=O)NC3=O)C4=O)nc21]

The title compound was prepared in a manner analogous to Example 1 by reductive amination between 4-hydroxy-piperidine and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₇H₂₉N₅O₄, 487.5; m/z found, 488.3 [M+H]⁺. ¹H NMR (400 MHZ, DMSO) δ 11.02 (s, 1H), 8.37 (s, 1H), 8.33 (d, J=8.0 Hz, 1H), 7.84 (d, J=8.0 Hz, 1H), 7.74 (s, 1H), 7.56 (d, J=3.2 Hz, 1H), 6.67 (d, J=3.0 Hz, 1H), 5.16 (dd, J=13.3, 5.1 Hz, 1H), 4.57 (d, J=17.2 Hz, 2H), 4.44 (d, J=17.3 Hz, 1H), 3.90 (s, 3H), 3.83 (s, 2H), 3.48 (s, 1H), 3.00-2.89 (m, 1H), 2.76 (s, 2H), 2.63 (d, J=17.0 Hz, 1H), 2.47-2.37 (m, 1H), 2.16 (s, 2H), 2.05 (dd, J=10.8, 5.5 Hz, 1H), 1.73 (d, J=9.5 Hz, 2H), 1.45 (d, J=9.1 Hz, 2H).

### Example 306:3-(5-(4-((3-hydroxyazetidin-1-yl)methyl)-1-methyl-1H-pyrrolo[2,3-b]pyridin-6-yl)-1-oxoisoindolin-2-yl) piperidine-2,6-dione

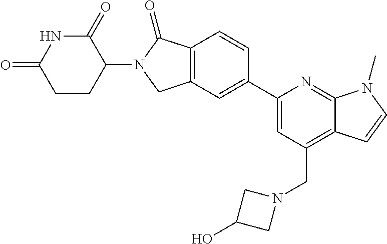 [SMILES: Cn1ccc2c(CN3CC(O)C3)cc(-c3ccc4c(c3)CN(C3CCC(=O)NC3=O)C4=O)nc21]

The title compound was prepared in a manner analogous to Example 1 by reductive amination between azetidin-3-ol and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₅H₂₅N₅O₄, 459.5; m/z found, 460.3 [M+H]⁺. ¹H NMR (400 MHZ, DMSO) δ 11.01 (s, 1H), 8.37 (s, 1H), 8.33 (d, J=8.0 Hz, 1H), 8.14 (s, 1H), 7.84 (d, J=8.0 Hz, 1H), 7.70 (s, 1H), 7.57 (d, J=3.4 Hz, 1H), 6.61 (d, J=3.4 Hz, 1H), 5.37 (s, 1H), 5.16 (dd, J=13.2, 5.2 Hz, 1H), 4.57 (d, J=17.3 Hz, 1H), 4.44 (d, J=17.4 Hz, 1H), 4.30-4.23 (m, 1H), 3.97 (s, 2H), 3.90 (s, 3H), 3.63 (t, J=6.9 Hz, 2H), 2.99-2.89 (m, 3H), 2.63 (d, J=17.3 Hz, 1H), 2.47-2.38 (m, 1H), 2.08-2.01 (m, 1H).

### Example 307:3-(1-oxo-5-(7-(pyrrolidin-1-ylmethyl)-1H-pyrazolo[4,3-b]pyridin-5-yl) isoindolin-2-yl) piperidine-2,6-dione

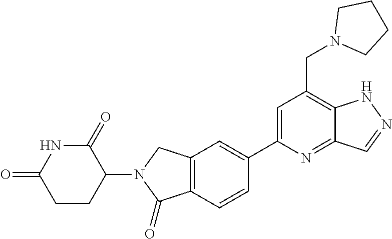 [SMILES: O=C1CCC(N2Cc3cc(-c4cc(CN5CCCC5)c5[nH]ncc5n4)ccc3C2=O)C(=O)N1]

The title compound was prepared in a manner analogous to Example 1 by reductive amination between pyrrolidine and 5-chloro-1H-pyrazolo[4,3-b]pyridine-7-carbaldehyde (commercially available) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₄H₂₄N₆O₃, 444.5; m/z found, 445.2 [M+H]⁺. ¹H NMR (400 MHZ, DMSO) § 13.60-13.21 (s, 1H), 11.02 (s, 1H), 8.37 (s, 2H), 8.28 (d, J=8.6 Hz, 1H), 8.22 (s, 1H), 7.98 (s, 1H), 7.85 (d, J=8.2 Hz, 1H), 5.16 (dd, J=13.5, 5.1 Hz, 1H), 4.57 (d, J=17.6 Hz, 1H), 4.44 (d, J=17.3 Hz, 1H), 4.03 (s, 2H), 2.92 (d, J=12.6 Hz, 1H), 2.65 (s, 1H), 2.58 (s, 4H), 2.43 (s, 1H), 2.07 (s, 1H), 1.76 (s, 4H).

### Example 308:3-(5-(4-((3-azabicyclo[3.2.1]octan-3-yl)methyl)-1-methyl-1H-pyrrolo[2,3-b]pyridin-6-yl)-1-oxoisoindolin-2-yl) piperidine-2,6-dione

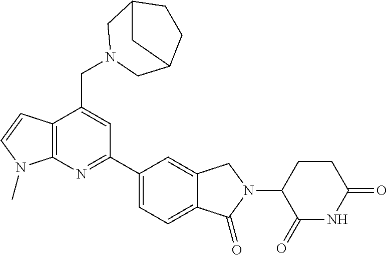 [SMILES: Cn1ccc2c(CN3CC4CCC(C4)C3)cc(-c3ccc4c(c3)CN(C3CCC(=O)NC3=O)C4=O)nc21]

The title compound was prepared in a manner analogous to Example 1 by reductive amination between 3-azabicyclo[3.2.1]octane and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₉H₃₁N₅O₃, 497.6; m/z found, 498.1 [M+H]⁺. ¹H NMR (400 MHZ, DMSO) δ 11.01 (s, 1H), 8.36 (s, 1H), 8.32 (d, J=8.1 Hz, 1H), 7.84 (d, J=8.0 Hz, 1H), 7.72 (s, 1H), 7.55 (d, J=3.4 Hz, 1H), 6.68 (d, J=3.4 Hz, 1H), 5.16 (dd, J=13.2, 5.1 Hz, 1H), 4.51 (dd, J=53.8, 17.2 Hz, 2H), 3.90 (s, 3H), 3.79 (s, 2H), 3.01-2.86 (m, 1H), 2.64 (d, J=7.3 Hz, 3H), 2.44 (dd, J=13.1, 4.5 Hz, 1H), 2.17-2.03 (m, 5H), 1.70 (d, J=6.6 Hz, 2H), 1.54 (d, J=4.5 Hz, 2H), 1.44 (d, J=11.3 Hz, 1H), 1.34 (d, J=10.8 Hz, 1H).

### Example 309:1-((6-(2-(2,6-dioxopiperidin-3-yl)-1-oxoisoindolin-5-yl)-1-methyl-1H-pyrrolo[2,3-b]pyridin-4-yl)methyl) piperidine-4-carbonitrile

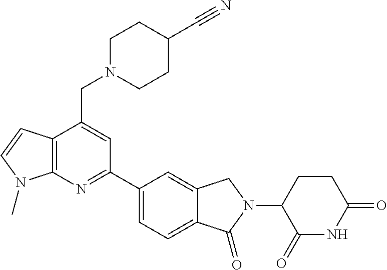 [SMILES: Cn1ccc2c(CN3CCC(C#N)CC3)cc(-c3ccc4c(c3)CN(C3CCC(=O)NC3=O)C4=O)nc21]

The title compound was prepared in a manner analogous to Example 1 by reductive amination between piperidine-4-carbonitrile and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₈H₂₈N₆O₃, 496.5; m/z found, 497.1 [M+H]⁺. ¹H NMR (400 MHZ, DMSO) δ 11.01 (s, 1H), 8.38 (s, 1H), 8.34 (d, J=8.2 Hz, 1H), 7.84 (d, J=8.0 Hz, 1H), 7.74 (s, 1H), 7.56 (s, 1H), 6.68 (d, J=2.4 Hz, 1H), 5.16 (dd, J=13.2, 4.9 Hz, 1H), 4.51 (dd, J=52.4, 17.3 Hz, 2H), 3.91 (s, 3H), 3.85 (s, 2H), 3.01-2.86 (m, 2H), 2.63 (d, J=14.9 Hz, 3H), 2.46-2.32 (m, 3H), 2.07-2.01 (m, 1H), 1.88 (s, 2H), 1.76 (s, 2H).

### Example 310:3-(5-(1-methyl-7-(pyrrolidin-1-ylmethyl)-1H-pyrazolo[4,3-b]pyridin-5-yl)-1-oxoisoindolin-2-yl) piperidine-2,6-dione

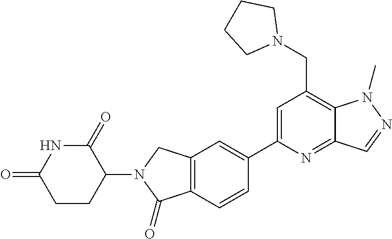 [SMILES: Cn1ncc2nc(-c3ccc4c(c3)CN(C3CCC(=O)NC3=O)C4=O)cc(CN3CCCC3)c21]

The title compound was prepared in a manner analogous to Example 1 by reductive amination between pyrrolidine and 5-chloro-1-methyl-1H-pyrazolo[4,3-b]pyridine-7-carbaldehyde (Intermediate 121a) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₅H₂₆N₆O₃, 458.5; m/z found, 459.3 [M+H]⁺. ¹H NMR (400 MHZ, DMSO) § 11.02 (s, 1H), 8.40 (s, 1H), 8.35-8.27 (m, 2H), 8.00 (s, 1H), 7.85 (d, J=8.0 Hz, 1H), 5.16 (dd, J=13.3, 5.1 Hz, 1H), 4.57 (d, J=17.4 Hz, 1H), 4.45 (d, J=17.3 Hz, 1H), 4.36 (s, 3H), 4.10 (s, 2H), 3.03-2.84 (m, 1H), 2.63 (d, J=18.4 Hz, 1H), 2.56 (s, 4H), 2.49-2.39 (m, 1H), 2.16-1.92 (m, 1H), 1.74 (s, 4H).

### Example 311:3-(5-(1-methyl-4-((8-methyl-3,8-diazabicyclo[3.2.1]octan-3-yl)methyl)-1H-pyrrolo[2,3-b]pyridin-6-yl)-1-oxoisoindolin-2-yl) piperidine-2,6-dione

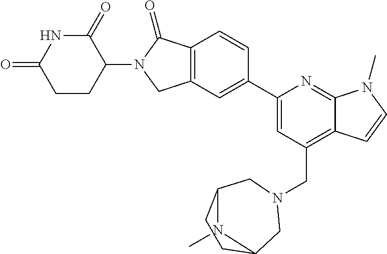 [SMILES: CN1C2CCC1CN(Cc1cc(-c3ccc4c(c3)CN(C3CCC(=O)NC3=O)C4=O)nc3c1ccn3C)C2]

The title compound was prepared in a manner analogous to Example 1 by reductive amination between 8-methyl-3,8-diazabicyclo[3.2.1]octane and 6-chloro-1-methyl-1H-pyrrolo[2,3-b]pyridine-4-carbaldehyde (Intermediate 8) followed by Suzuki coupling with 3-(1-oxo-5-(4,4,5,5-tetramethyl-1,3,2-dioxaborolan-2-yl) isoindolin-2-yl) piperidine-2,6-dione

(Intermediate 13) as a brown solid. LC-MS (ESI): mass calcd. for C₂₉H₃₂N₆O₃, 512.6; m/z found, 513.4 [M+H]⁺. ¹H NMR (400 MHZ, DMSO) δ 11.02 (s, 1H), 8.37 (s, 1H), 8.33 (d, J=8.0 Hz, 1H), 8.29 (s, 1H), 7.85 (d, J=8.0 Hz, 1H), 7.72 (s, 1H), 7.57 (d, J=3.4 Hz, 1H), 6.68 (d, J=3.4 Hz, 1H), 5.15 (dd, J=13.2, 5.0 Hz, 1H), 4.58 (d, J=17.3 Hz, 1H), 4.44 (d, J=17.3 Hz, 1H), 3.91 (s, 3H), 3.80 (s, 2H), 3.10 (s, 2H), 2.92 (dd, J=17.6, 4.9 Hz, 1H), 2.63 (d, J=17.6 Hz, 1H), 2.56 (d, J=8.4 Hz, 2H), 2.44 (dd, J=13.2, 4.8 Hz, 1H), 2.37 (s, 1H), 2.34 (s, 1H), 2.21 (s, 3H), 2.09-2.02 (m, 1H), 1.88 (d, J=4.8 Hz, 2H), 1.79 (d, J=7.0 Hz, 2H).

### Example 312:3-(1-oxo-5-(7-(pyrrolidin-1-ylmethyl) imidazo[1,5-a]pyridin-1-yl) isoindolin-2-yl) piperidine-2,6-dione

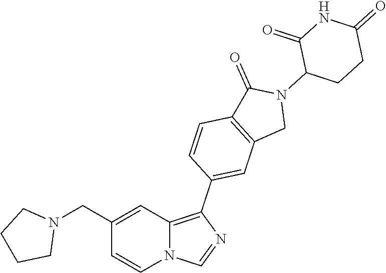 [SMILES: O=C1CCC(N2Cc3cc(-c4ncn5ccc(CN6CCCC6)cc45)ccc3C2=O)C(=O)N1]

The title compound was prepared in a manner analogous to Example 1 by reductive amination between pyrrolidine and 1-bromoimidazo[1,5-a]pyridine-7-carbaldehyde (Intermediate 123) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₅H₂₅N₅O₃, 443.2; m/z found, 444.3 [M+H]⁺. ¹H NMR (400 MHz, DMSO) δ 11.01 (s, 1H), 8.50 (s, 1H), 8.38 (d, J=7.4 Hz, 1H), 8.32 (s, 2H), 8.12 (s, 1H), 8.07 (d, J=8.1 Hz, 1H), 7.90 (s, 1H), 7.77 (d, J=8.0 Hz, 1H), 6.79 (d, J=7.2 Hz, 1H), 5.14 (m, 1H), 4.54 (d, J=17.2 Hz, 1H), 4.41 (d, J=17.2 Hz, 1H), 3.61 (s, 2H), 2.95 (dd, J=22.3, 9.2 Hz, 1H), 2.63 (d, J=15.3 Hz, 1H), 2.50-2.46 (m, 4H), 2.44-2.39 (m, 1H), 2.08-1.99 (m, 1H), 1.72 (s, 4H).

### Example 313:3-(5-(1-methyl-4-(pyrrolidin-1-ylmethyl)-1H-pyrazolo[3,4-b]pyridin-6-yl)-1-oxoisoindolin-2-yl) piperidine-2,6-dione

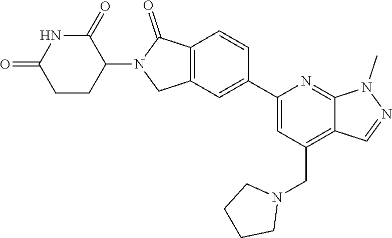

The title compound was prepared in a manner analogous to Example 1 by reductive amination between pyrrolidine and 6-chloro-1-methyl-1H-pyrazolo[3,4-b]pyridine-4-carbaldehyde (Intermediate 122a) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₅H₂₆N₆O₃, 458.5; m/z found, 459.3 [M+H]⁺. ¹H NMR (400 MHZ, DMSO) δ 11.01 (s, 1H), 8.44 (s, 1H), 8.38 (d, J=8.2 Hz, 1H), 8.29 (s, 1H), 7.92-7.82 (m, 2H), 5.16 (dd, J=13.3, 5.1 Hz, 1H), 4.59 (d, J=17.5 Hz, 1H), 4.46 (d, J=17.3 Hz, 1H), 4.13 (s, 3H), 4.04 (s, 2H), 2.92 (d, J=13.1 Hz, 1H), 2.63 (d, J=17.5 Hz, 1H), 2.55 (s, 4H), 2.43 (s, 1H), 2.03 (dd, J=5.9, 2.8 Hz, 1H), 1.75 (s, 4H).

### Example 314:3-(5-(2-methyl-7-(pyrrolidin-1-ylmethyl)-2H-pyrazolo[4,3-b]pyridin-5-yl)-1-oxoisoindolin-2-yl) piperidine-2,6-dione

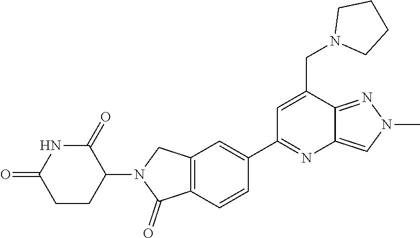

The title compound was prepared in a manner analogous to Example 1 by reductive amination between pyrrolidine and 5-chloro-2-methyl-2H-pyrazolo[4,3-b]pyridine-7-carbaldehyde (Intermediate 121b) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₅H₂₆N₆O₃, 458.5; m/z found, 459.3 [M+H]⁺. ¹H NMR (400 MHZ, DMSO) § 11.02 (s, 1H), 8.71 (s, 1H), 8.34 (s, 1H), 8.25 (d, J=8.0 Hz, 1H), 8.15 (s, 1H), 7.89 (s, 1H), 7.85 (d, J=8.0 Hz, 1H), 5.16 (dd, J=13.2, 5.1 Hz, 1H), 4.57 (d, J=17.3 Hz, 1H), 4.45 (d, J=17.3 Hz, 1H), 4.25 (s, 3H), 4.08 (s, 2H), 3.05-2.83 (m, 1H), 2.63 (s, 5H), 2.49-2.37 (m, 1H), 2.17-1.93 (m, 1H), 1.77 (s, 4H).

### Example 315:3-(5-(2-methyl-4-(pyrrolidin-1-ylmethyl)-2H-pyrazolo[3,4-b]pyridin-6-yl)-1-oxoisoindolin-2-yl) piperidine-2,6-dione

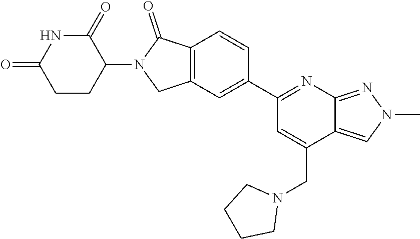

The title compound was prepared in a manner analogous to Example 1 by reductive amination between pyrrolidine and 6-chloro-2-methyl-2H-pyrazolo[3,4-b]pyridine-4-carbaldehyde (Intermediate 122b) followed by Suzuki coupling with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₅H₂₆N₆O₃, 458.5; m/z found, 459.3 [M+H]⁺. ¹H NMR (400 MHZ, DMSO) § 11.02 (s, 1H), 8.55 (s, 1H), 8.40 (s, 1H), 8.31 (d, J=8.1 Hz, 1H), 7.87 (d, J=8.0 Hz, 1H), 7.73 (s, 1H), 5.19-5.14 (m, 1H), 4.58 (d, J=17.3 Hz, 1H), 4.46 (d, J=17.4 Hz, 1H), 4.23 (s, 3H), 3.96 (s, 2H), 2.93 (dd, J=21.6, 9.5 Hz, 1H), 2.63 (d, J=17.4 Hz, 1H), 2.55 (s, 2H), 2.45 (dd, J=13.0, 4.3 Hz, 3H), 2.09-2.02 (m, 1H), 1.76 (s, 4H).

### Example 316:3-(5-(2-cyclobutyl-4-(pyrrolidin-1-ylmethyl)-2H-pyrazolo[3,4-b]pyridin-6-yl)-1-oxoisoindolin-2-yl) piperidine-2,6-dione

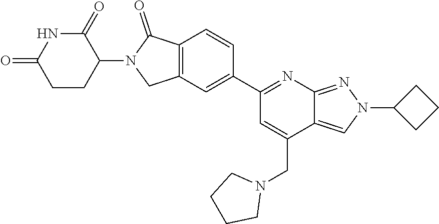

The title compound was prepared in a manner analogous to Example 1 by Suzuki coupling of 6-chloro-2-cyclobutyl-4-(pyrrolidin-1-ylmethyl)-2H-pyrazolo[3,4-b]pyridine (Example 206, side product) with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₈H₃₀N₆O₃, 498.6; m/z found, 499.3 [M+H]⁺. ¹H NMR (400 MHZ, DMSO) δ 11.02 (s, 1H), 8.64 (d, J=8.9 Hz, 1H), 8.41 (s, 1H), 8.32 (d, J=8.1 Hz, 1H), 8.14 (s, 1H), 7.87 (d, J=8.0 Hz, 1H), 7.81 (s, 1H), 5.27-5.20 (m, 1H), 5.16 (dd, J=13.2, 5.0 Hz, 1H), 4.58 (d, J=17.5 Hz, 1H), 4.45 (d, J=17.4 Hz, 1H), 4.11 (s, 2H), 2.92 (dd, J=12.9, 4.5 Hz, 1H), 2.76-2.63 (m, 6H), 2.60 (s, 1H), 2.55 (s, 2H), 2.43 (d, J=4.3 Hz, 1H), 2.09-2.01 (m, 1H), 1.93 (ddd, J=13.7, 8.6, 3.4 Hz, 2H), 1.80 (s, 4H).

### Example 317:3-(5-(2-(oxetan-3-yl)-4-(pyrrolidin-1-ylmethyl)-2H-pyrazolo[3,4-b]pyridin-6-yl)-1-oxoisoindolin-2-yl) piperidine-2,6-dione

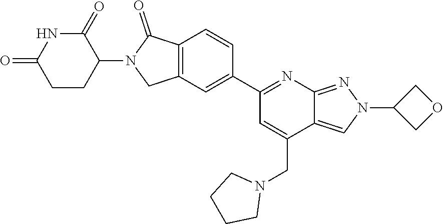

The title compound was prepared in a manner analogous to Example 1 Suzuki coupling of 6-chloro-2-(oxetan-3-yl)-4-(pyrrolidin-1-ylmethyl)-2H-pyrazolo[3,4-b]pyridine (Exxample 203, Step A, side product) with 3-(1-oxo-5-(4,4,5,5-tetramethyl-1,3,2-dioxaborolan-2-yl) isoindolin-2-yl) piperidine-2,6-dione (Intermediate 13) as a brown solid. LC-MS (ESI): mass calcd. for C₂₇H₂₈N₆O₄, 500.6; m/z found, 501.3 [M+H]⁺. ¹H NMR (400 MHZ, DMSO) δ 11.02 (s, 1H), 8.71 (s, 1H), 8.44 (s, 1H), 8.34 (d, J=7.7 Hz, 1H), 7.88 (d, J=8.0 Hz, 1H), 7.78 (s, 1H), 5.96 (t, J=7.0 Hz, 1H), 5.17 (dd, J=13.1, 5.1 Hz, 1H), 5.08 (d, J=6.1 Hz, 4H), 4.59 (d, J=17.2 Hz, 1H), 4.46 (d, J=17.4 Hz, 1H), 3.97 (s, 2H), 2.92 (d, J=12.3 Hz, 1H), 2.62 (d, J=18.9 Hz, 3H), 2.46-2.39 (m, 3H), 2.05 (d, J=5.1 Hz, 1H), 1.77 (s, 4H).

### Example 318:3-(5-(3-amino-7-(pyrrolidin-1-ylmethyl)-2H-pyrazolo[4,3-b]pyridin-5-yl)-4-fluoro-1-oxoisoindolin-2-yl) piperidine-2,6-dione

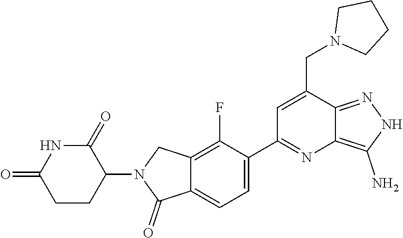 [SMILES: Nc1[nH]nc2c(CN3CCCC3)cc(-c3ccc4c(c3F)CN(C3CCC(=O)NC3=O)C4=O)nc12]

The title compound was prepared in a manner analogous to Example 173 by reductive amination between pyrrolidine and 3-amino-5-chloro-1-((2-(trimethylsilyl)ethoxy)methyl)-1H-pyrazolo[4,3-b]pyridine-7-carbaldehyde (Intermediate 27) followed by Suzuki coupling with 3-(4-fluoro-1-oxo-5-(4,4,5,5-tetramethyl-1,3,2-dioxaborolan-2-yl) isoindolin-2-yl) piperidine-2,6-dione (Intermediate 14) and de-protection of N-SEM group as a yellow solid. LC-MS (ESI): mass calced for: C₂₄H₂₄FN703, 477.5; m/z found, 478.2 [M+H]⁺. ¹H NMR (400 MHz, DMSO) δ 12.17 (s, 1H), 11.05 (s, 1H), 10.04 (s, 1H), 8.18 (t, J=7.3 Hz, 1H), 7.96 (s, 1H), 7.76 (d, J=7.8 Hz, 1H), 5.71 (s, 2H), 5.18 (dd, J=13.3, 4.6 Hz, 1H), 4.67 (d, J=17.1 Hz, 3H), 4.51 (d, J=17.6 Hz, 1H), 3.51 (s, 2H), 3.20 (s, 2H), 2.94 (d, J=11.5 Hz, 1H), 2.62 (s, 1H), 2.47 (d, J=1.9 Hz, 1H), 2.08 (s, 3H), 1.90 (s, 2H).

### II. BIOLOGICAL ASSAYS

In vitro Assay: IC₅₀ Measurements for binding to CRBN/DDB1 (Table E1)

The binding potency was determined using HTRF assay technology (Perkin Elmer). Compounds were serially diluted in DMSO and 0.2 μL volume was transferred to white 384-well plate. The reaction was conducted in total volume of 20 μL with addition of 2 nM His tagged CRBN+DDB−DLS7+CXU4 (Wuxi, catalogue #RP210521GA) to compounds followed by addition of 60 nM Fluorescent probe Cy5-labeled Thalidomide (Tenova Pharma, catalogue #T52461), and 0.4 nM of MAb Anti-6HIS Tb cryptate Gold (Cisbio, catalogue #61HI2TLA in the assay buffer (50 mM HEPES pH 7.5, 1 mM TCEP, 0.01% Brij-35, 50 mM NaCl, and 0.1% BSA). After one hour incubation at room temperature, the HTRF signals were read on Envision reader (Perkin Elemer). Data was analyzed using XLfit using four parameters dose response curve to determine IC₅₀s. A: IC₅₀<0.1 μM; B: 0.1 μM<IC₅₀<0.5 M; C: 0.5 μM<IC₅₀<1 μM; D: 1 μM<IC₅₀<5 μM; E: 5 μM<IC₅₀<10 μM

TABLE E1
CRBN binding IC₅₀

| | CRBN Binding | | CRBN Binding |
|---|---|---|---|
| Cmpd No. | IC₅₀ (μM) | Cmpd No. | IC₅₀ (μM) |
| 1 | B | 41 | B |
| 2 | B | 42 | A |
| 3 | B | 43 | A |
| 4 | D | 44 | B |
| 5 | D | 45 | B |
| 6 | B | 46 | B |
| 7 | C | 47 | A |
| 8 | D | 48 | A |
| 9 | C | 49 | B |
| 10 | C | 50 | D |
| 11 | C | 51 | B |
| 12 | B | 52 | C |
| 13 | B | 53 | E |
| 14 | B | 54 | D |
| 15 | D | 55 | B |
| 16 | C | 56 | A |
| 17 | D | 57 | B |
| 18 | C | 58 | C |
| 19 | D | 59 | B |
| 20 | D | 60 | C |
| 21 | B | 61 | C |
| 22 | D | 62 | B |
| 23 | D | 63 | B |
| 24 | D | 64 | B |
| 25 | B | 65 | B |
| 26 | B | 66 | B |
| 27 | C | 67 | C |
| 28 | C | 68 | D |
| 29 | D | 69 | B |
| 30 | D | 70 | D |
| 31 | B | 71 | B |
| 32 | B | 72 | B |
| 33 | B | 73 | B |
| 34 | A | 74 | C |
| 35 | B | 75 | B |
| 36 | B | 76 | D |
| 37 | A | 77 | D |
| 38 | B | 78 | C |
| 39 | A | 79 | B |
| 40 | B | 80 | B |
| 81 | E | 171 | B |
| 82 | E | 172 | D |
| 83 | D | 173 | D |
| 84 | A | 174 | C |
| 85 | B | 175 | D |
| 87 | B | 176 | C |
| 88 | D | 177 | C |
| 89 | C | 178 | C |
| 90 | B | 179 | E |
| 91 | C | 184 | C |
| 92 | A | 185 | C |
| 93 | B | 186 | B |
| 94 | A | 187 | B |
| 95 | B | 188 | C |
| 96 | B | 189 | B |
| 97 | A | 190 | B |
| 98 | A | 191 | D |
| 99 | B | 192 | D |
| 100 | B | 193 | D |
| 101 | B | 194 | D |
| 102 | B | 195 | D |
| 103 | B | 196 | D |
| 104 | C | 197 | C |
| 105 | C | 200 | B |
| 106 | B | 201 | C |
| 107 | B | 204 | D |
| 108 | C | 205 | C |
| 109 | B | 207 | B |
| 110 | C | 208 | A |
| 111 | B | 209 | D |
| 112 | B | 210 | C |
| 113 | D | 211 | C |
| 114 | D | 212 | A |
| 115 | C | 213 | B |
| 116 | B | 214 | B |
| 117 | B | 215 | B |
| 118 | C | 216 | D |
| 119 | D | 217 | C |
| 120 | B | 218 | C |
| 121 | D | 219 | D |
| 122 | D | 221 | B |
| 123 | B | 222 | B |
| 124 | D | 223 | B |
| 125 | B | 224 | D |
| 126 | B | 225 | C |
| 127 | B | 226 | B |
| 128 | B | 227 | C |
| 129 | B | 228 | B |
| 130 | B | 229 | B |
| 131 | D | 230 | B |
| 132 | E | 231 | B |
| 133 | B | 232 | C |
| 139 | C | 233 | D |
| 140 | B | 234 | D |
| 141 | D | 235 | E |
| 142 | C | 236 | D |
| 143 | D | 237 | D |
| 144 | B | 238 | D |
| 145 | D | 239 | D |
| 146 | B | 299 | C |
| 147 | B | 300 | D |
| 148 | B | 301 | D |
| 149 | D | 302 | D |
| 154 | C | 170 | C |
| 155 | B | 303 | D |
| 156 | D | 304 | D |
| 157 | C | 305 | D |
| 158 | B | 306 | B |
| 159 | B | 307 | B |
| 160 | A | 308 | B |
| 161 | B | 309 | D |
| 162 | B | 310 | C |
| 163 | B | 311 | C |
| 164 | B | 312 | D |
| 165 | B | 313 | D |
| 166 | B | 314 | B |
| 167 | B | 315 | B |
| 168 | C | 316 | B |
| 169 | A | 317 | B |

In vitro IKZF2 Western Blot Assay

Jurkat cells (ATCC, Cat #HB-8065) were cultured in RPMI1640+10% FBS+1% P/S. Cells were treated at desired compound concentrations (0.001, 0.01, 0.1, 1, and 10 μM) and DMSO as vehicle control for 24 hrs. After 24 hr of treatment cells were collected by centrifugation and lysed in RIPA lysis buffer on ice for 10 min. After assessing protein concentration by BCA assay (Pierce), equal amounts of protein for each sample were loaded into 4-12% Bis-Tris gels (Invitrogen), transferred to PVDF membranes and immunoblotted with antibodies against IKZF2 and Actin (Cell Signaling). Membranes were developed on an Odyssey detection system (LI-COR Biosciences) after incubation with IRDye800-labeled goat anti-rabbit IgG and IRDye680-labeled goat anti-mouse IgG (LI-COR) secondary antibodies. Band intensities were quantified using ImageStudio software. Data was further analyzed using XLfit. Percentage of IKZF2 protein degradation: A (>50%); B (30-50%); C(<30%) (Table 2).

TABLE 2
IKZF2 degradation by Western Blot

| Cmpd | IKZF2 degradation (%) @ μM | | | | |
|---|---|---|---|---|---|
| No. | 0.001 | 0.01 | 0.1 | 1 | 10 |
| 21 | B | A | A | A | A |
| 26 | C | C | B | B | B |
| 33 | C | A | A | A | A |
| 34 | B | B | A | A | A |
| 35 | C | C | C | C | C |
| 36 | B | B | C | C | C |
| 37 | C | C | C | C | C |
| 38 | C | C | C | C | C |
| 39 | C | C | C | C | C |
| 40 | C | C | C | C | B |
| 41 | C | C | C | C | C |
| 42 | C | C | C | C | B |
| 43 | C | C | C | C | C |
| 44 | C | C | B | C | B |
| 45 | B | C | C | A | C |
| 46 | C | C | B | A | A |
| 47 | B | B | C | C | C |
| 48 | C | C | C | C | C |
| 49 | C | C | C | C | C |

In vitro IKZF2 FACS Assay

Jurkat cells (ATCC, Cat #HB-8065) were cultured in RPMI1640+10% FBS+1% P/S. Cells were treated at desired compound concentrations (0.05 to 10 μM) and DMSO as vehicle control for 24 hrs. After 24 hrs of drug treatment cells were washed, fixed (3.7% PFA, and permeabilized with perm buffer (0.3% Triton X-100 in 1% BSA Solution). Subsequently, cells were stained with IKZF2 (1:100, Cell signaling) primary antibody and Alexa 488-labelend anti-rabbit IgG (1:200, Cell Signaling) secondary antibodies in staining buffer (1% BSA in PBS). Cells were images on iQue Flowcytometer and IKZF2 levels were quantified using iQue software. Data was further analyzed using XLfit using four parameters dose response curve to determine DC₅₀ and Dₘₐₓ. The half maximal degradation concentration values (DC₅₀) and maximal degradation percentage (Dₘₐₓ, %) of IKZF2 are summarized in Table 3. DC₅₀, A: <1 nM; B: 1-10 nM; C: 10-100 nM; D: 100-1000 nM; E: >1000 nM. Dₘₐₓ, A: >80%; B: 60-80%; C: 40-60%; D: 20-40%; E: <20%.

TABLE 3
IKZF2 degradation by FACS

| | |
|---|---|
| Cmpd | IKZF2 FACS |

| No. | DC₅₀ (nM) | Dₘₐₓ (%) |
|---|---|---|
| 1 | C | B |
| 2 | D | C |
| 3 | C | C |
| 4 | E | D |
| 5 | C | C |
| 6 | C | B |
| 7 | C | C |
| 8 | C | C |
| 9 | C | C |
| 10 | B | C |
| 11 | D | C |
| 12 | D | C |
| 13 | C | D |
| 14 | C | C |
| 15 | C | D |
| 16 | C | C |
| 17 | C | D |
| 18 | D | C |
| 19 | D | D |
| 20 | C | D |
| 22 | E | D |
| 23 | C | D |
| 24 | C | D |
| 25 | E | E |
| 27 | C | C |
| 31 | E | E |
| 51 | E | D |
| 52 | C | C |
| 53 | E | D |
| 54 | A | A |
| 55 | A | A |
| 56 | B | B |
| 105 | B | A |
| 116 | B | A |
| 117 | B | A |
| 119 | B | A |
| 121 | C | A |
| 128 | B | A |
| 129 | B | A |
| 151 | B | A |
| 158 | C | A |
| 161 | C | B |
| 162 | C | A |
| 163 | B | A |
| 164 | C | A |
| 170 | C | B |
| 176 | B | B |
| 180 | C | A |
| 181 | B | A |
| 182 | C | B |
| 186 | B | A |
| 187 | B | A |
| 189 | B | A |
| 193 | B | C |
| 199 | C | B |
| 203 | B | A |
| 205 | D | B |
| 206 | B | A |
| 209 | C | A |
| 210 | B | A |
| 211 | C | B |
| 212 | C | A |
| 215 | C | A |
| 216 | D | B |
| 218 | C | B |
| 220 | C | C |
| 221 | B | A |
| 222 | B | A |
| 223 | B | A |
| 226 | B | A |
| 227 | C | A |
| 228 | C | B |
| 229 | B | B |
| 230 | B | A |
| 232 | C | B |
| 236 | E | E |
| 237 | C | C |
| 239 | C | B |
| 240 | B | A |
| 242 | B | A |
| 245 | B | C |
| 246 | B | A |
| 248 | A | A |
| 249 | B | A |
| 250 | B | B |
| 254 | C | B |
| 255 | B | A |
| 260 | B | A |
| 261 | B | A |
| 264 | C | C |
| 265 | B | B |
| 266 | B | A |
| 268 | B | A |
| 269 | B | A |
| 270 | B | A |
| 272 | B | A |
| 274 | B | B |
| 275 | C | B |
| 276 | B | A |
| 277 | B | A |
| 280 | C | B |
| 282 | C | A |
| 283 | B | A |
| 284 | C | B |
| 288 | C | B |
| 289 | B | A |
| 290 | C | B |
| 291 | A | A |
| 292 | B | A |
| 293 | B | A |
| 295 | B | A |
| 307 | B | A |
| 314 | B | A |
| 318 | B | A |

### In Vitro IKZF2 HiBit Assay

The HiBiT protein tagging system was applied to modified HEK293T Flp-in-HiBiT cells (polyclone) via a CRISPR/Cas9-mediated insertion of the HiBiT peptide tag (Promega™) to the N-terminus of the IKZF2 gene locus (Neon™ transfection system). Test and reference compounds are diluted from 1 mM at 3 folds for 11 doses. 25 nL of diluted compound is transfered to assay plates (Corning3570) using ECHO550, the final DMSO concentration @ 0.1%. The cells are seeded in 3000/25 μL/well to compound plates. It is then incubated for 6 hrs in TC incubator. The amount of Nano-Glo® HiBiT lytic reagent needed to perform the desired experiments is calculated. The Nano-Glo® HiBiT lytic reagent is brought to room temperature. The LgBiT protein is diluted to 1:100 and the Nano-Glo® HiBiT lytic substrate is diluted to 1:50 into an appropriate volume of room temperature Nano-Glo® HiBiT lytic buffer. 15 μL of the detection reagent (or without LgBiT) is dispensed to corresponding well according to the layout. The plate is then shaked for 10 mins at room temperature. After briefly centrifuge, the plate is read on Envision. At the indicated timepoints, the Nano-Glo® HiBiT lytic detection system (Promega™) was utilized for detecting bioluminescence of the HiBiT tag in treated cells: abundance of the tag is proportionate to the level of luminescence. Following normalization to DMSO, dose-response curves were plotted (GraphPad Prism) to determine the concentration points at which 50% of HiBiT-Helios degradation was achieved by each compound. The extent of degradation (range of luminescence) from the highest to lowest concentration points was calculated to determine the Dₘₐₓ.

### In Vitro IKZF1 HiBit Assay

The HiBiT protein tagging system was applied to modified Cells: modified HEK293T Flp-in-HiBiT-IKZF1 stable cell line (polyclone) via a CRISPR/Cas9-mediated insertion of the HiBiT peptide tag (Promega™) to the N-terminus of the IKZF2 gene locus (Neon™ transfection system).

Test compound from 10 mM and reference compound (CC-92480 from 50 μM and I-57 from 10 mM) are diluted at 3 folds for 11 doses. 25 nL of diluted compound is transferred to assay plates (Corning3571) using ECHO550, the final DMSO concentration @ 0.1%. The cells are seeded in 3000/25 μL/well to compound plates. The plates are incubated for 6 hrs in TC incubator. The amount of Nano-Glo® HiBiT lytic reagent needed to perform the desired experiments is calculated. The Nano-Glo® HiBiT lytic reagent is brought to room temperature. The LgBiT protein is diluted to 1:100 and the Nano-Glo® HiBiT lytic substrate is brought to 1:50 into an appropriate volume of room temperature Nano-Glo® HiBiT lytic buffer. 15 μL of the detection reagent (or without LgBiT) is dispensed to corresponding well according to the layout. The plate is shaked for 10 mins at room temperature. After briefly centrifuge, the plateis read on Envision. At the indicated timepoints, the Nano-Glo® HiBiT lytic detection system (Promega™) was utilized for detecting bioluminescence of the HiBiT tag in treated cells: abundance of the tag is proportionate to the level of luminescence. Following normalization to DMSO, dose-response curves were plotted (GraphPad Prism) to determine the concentration points at which 50% of HiBiT-Ikaros degradation was achieved by each compound. The extent of degradation (range of luminescence) from the highest to lowest concentration points was calculated to determine the Dₘₐₓ.

The half maximal degradation concentration values (DC₅₀) and maximal degradation percentage (Dₘₐₓ, %) of IKZF2 and IKZF1 are summarized in Table 4. DC₅₀, A: <1 nM; B: 1-10 nM; C: 10-100 nM; D: 100-1000 nM; E: 1000-10000 nM; F: >10,000 nM. Dₘₐₓ, A: >80%; B: 60-80%; C: 40-60%; D: 20-40%; E: <20%.

TABLE 4
IKZF2 and IKZF1 degradation by HiBit

| Cmpd | IKZF2 HiBit | | IKZF1 HiBit | |
|---|---|---|---|---|
| No. | DC₅₀ (nM) | Dₘₐₓ (%) | DC₅₀ (nM) | Dₘₐₓ (%) |
| 1 | C | B | F | E |
| 3 | C | B | F | E |
| 6 | C | B | F | E |
| 8 | C | C | F | E |
| 10 | E | C | F | E |
| 30 | E | E | F | E |
| 52 | B | B | F | E |
| 54 | E | E | F | E |
| 55 | A | A | F | D |
| 56 | A | A | F | D |
| 57 | A | A | F | D |
| 58 | B | B | F | E |
| 59 | B | A | F | D |
| 60 | B | B | F | E |
| 61 | C | C | F | E |
| 62 | B | A | F | E |
| 63 | A | A | F | D |
| 64 | A | A | F | D |
| 65 | B | A | F | D |
| 66 | A | A | F | D |
| 67 | B | B | F | D |
| 68 | D | D | F | E |
| 69 | A | A | F | D |
| 70 | B | A | F | E |
| 71 | B | A | F | D |
| 72 | A | A | F | C |
| 73 | B | A | F | D |
| 74 | B | A | F | E |
| 75 | A | A | F | D |
| 76 | A | A | F | D |
| 77 | A | A | F | D |
| 78 | A | A | F | D |
| 79 | A | A | F | D |
| 80 | A | A | FE | D |
| 81 | A | A | C | C |
| 82 | D | D | F | E |
| 83 | B | A | F | E |
| 84 | B | A | C | D |
| 85 | B | A | B | D |
| 87 | D | E | F | E |
| 88 | C | A | F | E |
| 89 | D | D | F | E |
| 90 | B | A | C | D |
| 91 | B | A | C | D |
| 92 | B | A | F | E |
| 93 | A | A | C | D |
| 94 | B | B | F | E |
| 95 | B | B | F | E |
| 96 | B | A | C | D |
| 97 | C | C | F | E |
| 98 | B | A | F | E |
| 99 | C | C | F | E |
| 100 | A | A | C | D |
| 101 | B | A | F | E |
| 102 | A | A | C | D |
| 103 | B | A | C | D |
| 104 | B | A | F | D |
| 105 | B | A | E | D |
| 106 | B | A | F | E |
| 107 | C | C | F | E |
| 108 | B | B | F | E |
| 109 | A | A | B | D |
| 110 | B | A | F | E |
| 111 | C | B | F | E |
| 112 | A | A | C | D |
| 113 | A | B | C | D |
| 114 | C | D | F | E |
| 115 | C | B | F | E |
| 116 | A | A | B | D |
| 117 | B | A | F | E |
| 118 | B | C | F | E |
| 119 | A | A | E | D |
| 120 | C | A | D | D |
| 121 | B | A | F | E |
| 122 | D | D | B | D |
| 123 | B | A | C | D |
| 124 | B | A | C | D |
| 125 | C | A | D | D |
| 126 | A | A | B | C |
| 127 | B | A | D | D |
| 128 | A | A | C | D |
| 129 | A | A | C | D |
| 130 | B | C | F | E |
| 131 | C | B | F | E |
| 132 | C | C | F | E |
| 133 | B | B | F | E |
| 134 | B | A | F | E |
| 135 | B | A | F | E |
| 136 | C | D | F | E |
| 137 | B | A | C | D |
| 138 | B | A | D | D |
| 139 | B | B | C | D |
| 140 | A | D | F | E |
| 141 | B | A | F | E |
| 142 | B | A | F | E |
| 143 | B | A | F | E |
| 144 | B | A | C | D |
| 145 | D | E | F | E |
| 146 | B | A | B | C |
| 147 | B | C | F | E |
| 148 | B | B | F | E |
| 149 | D | D | F | E |
| 150 | C | B | F | E |
| 151 | B | A | F | E |
| 152 | C | A | D | D |
| 153 | A | A | C | C |
| 154 | B | B | F | E |
| 155 | B | A | C | D |
| 156 | B | A | C | D |
| 157 | B | A | F | E |
| 158 | C | B | F | E |
| 159 | B | B | C | D |
| 160 | C | C | F | E |
| 161 | B | B | F | E |
| 162 | C | B | D | D |
| 163 | B | B | D | D |
| 164 | C | B | D | D |
| 165 | C | B | F | E |
| 166 | B | C | F | E |
| 167 | C | B | D | D |
| 168 | D | B | E | D |
| 169 | B | A | C | C |
| 170 | B | B | F | E |
| 171 | B | B | F | E |
| 172 | C | B | D | D |
| 173 | C | B | D | D |
| 174 | B | B | C | D |
| 175 | C | B | F | E |
| 176 | B | A | C | D |
| 177 | B | B | C | D |
| 178 | B | B | D | D |
| 179 | A | A | C | C |
| 180 | B | A | B | D |
| 181 | B | A | C | C |
| 182 | B | A | F | E |
| 183 | B | A | C | C |
| 184 | B | B | E | D |
| 185 | D | C | F | E |
| 186 | B | A | F | E |
| 187 | B | A | F | E |
| 188 | B | B | F | E |
| 189 | B | A | C | D |
| 190 | C | C | F | E |
| 191 | B | B | F | E |
| 192 | C | B | F | E |
| 193 | B | A | C | D |
| 194 | B | A | F | E |
| 195 | C | B | F | E |
| 196 | D | C | F | E |
| 197 | B | B | F | E |
| 198 | C | C | F | E |
| 199 | B | A | C | D |
| 200 | D | B | D | D |
| 201 | C | B | E | D |
| 202 | B | A | B | C |
| 203 | B | A | C | D |
| 204 | E | E | F | E |
| 205 | C | B | E | D |
| 206 | B | A | F | E |
| 207 | C | B | F | E |
| 208 | D | B | F | E |
| 209 | B | A | F | E |
| 210 | B | A | D | C |
| 211 | C | B | F | E |
| 212 | B | B | B | D |
| 213 | D | C | B | D |
| 214 | D | B | F | E |
| 215 | C | A | D | D |
| 216 | C | B | D | D |
| 217 | B | B | F | E |
| 218 | B | B | D | D |
| 219 | C | C | D | D |
| 220 | C | B | F | E |
| 221 | B | A | F | E |
| 222 | B | A | C | D |
| 223 | A | B | B | D |
| 224 | A | B | C | C |
| 225 | A | D | F | E |
| 226 | A | A | F | E |
| 227 | B | B | F | E |
| 228 | B | B | F | E |
| 229 | B | B | F | E |
| 230 | B | B | F | E |
| 231 | C | C | B | D |
| 232 | B | B | F | E |
| 233 | F | E | F | E |
| 234 | C | B | E | D |
| 235 | B | B | D | D |
| 236 | C | D | F | E |
| 237 | C | B | B | D |
| 238 | A | B | F | E |
| 239 | B | B | F | E |
| 240 | B | A | F | E |
| 241 | C | C | F | E |
| 242 | B | A | F | E |
| 243 | C | B | D | D |
| 244 | C | C | F | E |
| 245 | B | C | F | E |
| 246 | B | A | F | E |
| 247 | C | C | F | E |
| 248 | A | A | D | D |
| 249 | B | A | F | E |
| 250 | B | B | F | E |
| 251 | C | B | F | E |
| 252 | C | C | F | E |
| 253 | C | C | F | E |
| 254 | B | B | F | E |
| 255 | B | A | C | D |
| 256 | C | A | F | E |
| 257 | B | A | B | D |
| 258 | C | B | F | E |
| 259 | F | E | F | E |
| 260 | B | A | C | B |
| 261 | B | A | C | B |
| 262 | B | B | F | E |
| 263 | B | C | F | E |
| 264 | B | B | F | E |
| 265 | B | A | F | E |
| 266 | B | A | C | C |
| 267 | B | B | F | E |
| 268 | A | A | C | C |
| 269 | B | A | C | D |
| 270 | A | A | F | E |
| 271 | B | A | D | D |
| 272 | B | A | C | C |
| 273 | F | E | F | E |
| 274 | A | A | E | D |
| 275 | B | B | F | E |
| 276 | B | A | F | E |
| 277 | A | A | F | E |
| 278 | F | E | F | E |
| 279 | B | B | F | E |
| 280 | C | B | F | E |
| 281 | C | B | F | E |
| 282 | B | A | E | D |
| 283 | B | A | F | E |
| 284 | B | A | C | D |
| 285 | B | D | F | E |
| 286 | B | B | F | E |
| 287 | C | B | D | D |
| 288 | B | A | F | E |
| 289 | B | A | C | C |
| 290 | B | B | F | E |
| 291 | A | A | C | C |
| 292 | B | A | C | D |
| 293 | B | A | F | E |
| 294 | E | D | F | E |
| 295 | A | A | C | C |
| 296 | A | A | B | B |
| 297 | F | E | F | E |
| 298 | A | D | F | E |
| 299 | B | B | F | E |
| 300 | C | B | F | E |
| 301 | A | A | C | D |
| 302 | A | A | B | D |
| 303 | B | A | C | D |
| 304 | B | A | F | E |
| 305 | B | A | F | E |
| 306 | C | A | D | D |
| 307 | B | A | E | D |
| 308 | B | A | B | D |
| 309 | B | A | E | D |
| 310 | A | A | F | E |
| 311 | B | B | F | E |
| 312 | C | D | F | E |
| 313 | A | A | C | D |
| 314 | B | A | C | D |
| 315 | C | A | C | D |
| 316 | B | A | C | D |
| 317 | B | B | F | E |
| 318 | B | A | C | B |

### IKZF2 Degradation and Evaluation of IL-2 Production

IKZF2 is important for immunosuppressive activity of regulatory T cells (T_{reg} cells), which is linked to interleukin-2 (IL-2) repression. IKZF2 binds to the IL-2 promoter in T_{reg} cells and suppresses transcriptional activation. IKZF2 knockdown suppresses FoxP3 binding to IL-2 promoter and results in higher IL-2 expression upon stimulation. Further, IKZF2 knockout leads to an unstable CD4 Treg phenotype in mice marked by production of effector cytokines and IKZF2 knockout in Tregs suppresses tumor growth. (Baine I. et al., J Immunol 190, 1008-1016 (2013); Nakagawa, H. et al. Proc National Acad Sci 113, 6248-6253 (2016); Yates, K., et al. Proc National Acad Sci 115, 201720447 (2018).

To measure whether IKZF2 degradation by the compounds of this disclosure impacts IL-2 production, Jurkat cells (ATCC, Cat #HB-8065) are treated with vehicle control (DMSO) or the compound for 16-24 hrs. After 16-24 hrs of treatment cells are stimulated with CD3/CD28 stimulation beads at a 3:1 ratio for 24 hrs. After 24 hrs, supernatants are collected and the concentration of IL-2 is measured using MSD V-PLEX Human IL-2 Kit (Cat #K₁₅₁QQD, Mesoscale). The compounds of this disclosure are expected to increase IL-2 production, and thereby increase anti-tumor immunity.

### IKZF2 Degradation in Primary Human T_{reg} Cells

To measure whether the compounds of this disclosure can induce degradation of IKZF2 in T_{reg} cells, human peripheral bone marrow cells (PBMCs) obtained from healthy donors purchased from Milestone Biological Science and Technology Company are treated with vehicle control (DMSO) or the compound for various time points (3-24 hrs). After desired treatment time, the cells are collected and stained with anti-CD3-APC-Cy7 (Clone SP34-2, BD), anti-CD4-FITC(Clone L200, BD), anti-CD45-BV510 (Clone HI30, Biolegend), and anti-CD25-BV421 (Clone BC96, Biolegend) in cell staining buffer (Biolegend, Cat #420201), washed and fixed with FOXP3 fix/perm buffer (Life Technologies, cat. #00-5523-00) followed by intracellular staining with anti-IKZF2-APC(Clone 22F₆, BioLegend), anti-Ikaros-PE-Cy7 (Clone 16B5C71, BioLegend), and anti-FOXP3-PE (clone 206D, Biolegend). Samples are acquired on a Thermo Attune NxT flow cytometer (Thermo Fisher Scientific). IKZF2 mean fluorescence intensity (MFI) and IKZF1 MFI are measured in Tregs (CD4+CD25+FOXP3+) cells. The compounds of this disclosure are expected to degrade IKZF2 in T_{reg} cells, thereby suppressing the action of T_{reg} cells.

### IKZF2 Degradation and T_{eff} Cell Proliferation

To measure whether the compounds of this disclosure can enhance effector T cell (T_{eff}) proliferation via suppression of T_{reg} cells, T_{reg} cells and T_{eff} cells from matched human donors are co-cultured in the presence of vehicle control (DMSO) or compound. T_{reg} cells are isolated from human peripheral bone marrow cells (PBMCs) obtained from healthy donors purchased from Milestone Biological Science and Technology Company. CD4 enrichment by negative selection followed by CD25 enrichment by positive selection are performed using the human CD4 T cell isolation kit (cat. #130-096-533) and human CD25 microbeads (cat. #130-092-983) from Miltenyi Biotec (Cambridge, MA) according to manufacturer's instructions. Isolated T_{regs} are expanded for 8-14 days in the presence of compound or DMSO, using T_{reg} expander beads (ThermoFisher, cat. #11129D) or T-cell activator beads (ThermoFisher, cat. #11161D) at a 4:1 or 3:1 ratio, respectively, in the presence of 500 U/mL rhIL-2. Expanded T_{reg} cells are dispensed in co-culture with carboxyfluorescein succinimidyl ester (CFSE)-labelled CD3+T-Cells from the matched donor at various T_{reg}: CD3+ T cell ratios in the presence of T-cell activator beads or soluble anti-CD3 antibody (30 ng/mL, OKT3, Thermofisher cat. #16-0037-81). After 3-5 days of incubation, proliferation of CD8+T_{eff} cells is assessed by analyzing CFSE dye dilution in CD8+ T-Cells (anti-CD8-PerCP/Cyanine5.5, clone SK1, Biolegend) using flow cytometry. Analysis is performed using a Thermo Attune NXT flow cytometer (Thermo Fisher Scientific). T_{eff} cells that proliferate during the co-culture are identified as having diluted CFSE and data are plotted as the proportion of CFSE low, proliferated cells in the final culture. The compounds of this disclosure are expected to suppress T_{reg} cells, thereby enhancing T_{eff} cell proliferation.

### In Vivo Pharmacology and Efficacy Studies

### Cynomolgus Moneys

To determine in vivo efficacy of the compounds of this disclosure, non naïve cynomolgus monkeys are treated with a single oral dose of vehicle or the compound. Whole blood from the treated monkeys is collected across time (e.g., various timepoints between 0 hr -96 hrs) and stained with anti-CD3-APC-Cy7 (Clone SP34-2, BD), anti-CD4-FITC(Clone L200, BD), anti-CD45-BV786 (Clone D058-1283, Biolegend), and anti-CD25-APC(Clone BC96, Biolegend) in cell staining buffer (Biolegend, Cat #420201), washed and fixed with FOXP3 fix/perm buffer (Life Technologies, cat. #00-5523-00) followed by intracellular staining with anti-IKZF2-PE (Clone 22F6, BioLegend) and anti-FOXP3-BV421 (clone 206D, Biolegend). Samples are acquired on a Thermo Attune NxT flow cytometer (Thermo Fisher Scientific). IKZF2 mean fluorescence intensity (MFI) is measured in T_{regs} (CD4+CD25+FOXP3+) cells. The compounds of this disclosure are expected to suppress IKZF2⁺T_{regs} in cynomolgus monkeys.

### Mice

To determine in vivo efficacy of the compounds of this disclosure, CRBN^{1391V} mice are treated with a single oral dose of vehicle or the compound. CRBN^{1391V} mice are used because a single amino acid difference within the CRBN-Immunomodulatory drug (IMiD) binding region renders mouse CRBN resistant to degradation by IMiDs. A change from Ile 391 to Val in mouse CRBN restores IMiD-induced degradation of IKZF3. Fink, E. C. et al. Blood 132, 1535-1544 (2018); Gemechu, Y. et al. P Natl Acad Sci Usa 115, 11802-11807 (2018).

1. IKZF2 degradation in mice: Various doses of the vehicle and compound are tested in the mice and analyzed across time (e.g., various timepoints between 0 hr-12 hrs) and analyzed using western blot assay to measure the percentage of IKZF2 remaining in tissues (e.g., spleen and thymus). Tissue is lysed in RIPA buffer (Cell Signaling, cat #9806) containing Halt™ protease/phosphatase inhibitor cocktail (Thermo, Cat #78440). After assessing protein concentration by BCA assay (Pierce), equal amounts of protein for each sample are loaded into 4-12% Bis-Tris gels (Invitrogen), transferred to nitrocellulose membranes and immunoblotted with antibodies against Helios (Cell Signaling, Cat #4247) and b-Actin (Cell Signaling, Cat #3700). Membranes are developed on an Odyssey detection system (LI-COR Biosciences) after incubation with IRDye800-labeled goat anti-rabbit IgG and IRDye680-labeled goat anti-mouse IgG (LI-COR) secondary antibodies. The compounds of this disclosure are expected to degrade IKZF2 in CRBN^{1391V} mice.

2. Tumor growth inhibition in mice: To develop cancer cell line xenografts, CRBN1391V mice are implanted with MC38 cells (ATCC) subcutaneously to induce tumor formation. MC38 cells (e.g., five million) in 50% Matrigel are injected subcutaneously into CRBN^{1391V} mice to induce tumor formation. Mice are treated with vehicle control (e.g., 5% DMSO, 10% solutol, 85% Water) or the compound once tumors reach ˜80-400 mm³, and sacrificed when tumor volume reached 2000 mm³ or at the end of the study (whichever occurs first). Tumor sizes and animal weights are measured 2-3 times per week. Tumor volume (mm³)═(length×width²)/2. Tumor growth inhibition is calculated using TGI (%)═(1−((Tₑ−T₀)/(Cₑ−C₀)))‘100, where Tₑ=Test tumor volume endpoint, T₀=Test tumor volume at start of dosing, Cₑ=Vehicle control tumor volume endpoint, C₀=Vehicle control tumor volume at start of dosing The compounds of this disclosure are expected to inhibit MC38 tumor growth in CRBN^{1391V} mice.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A compound of Formula II: 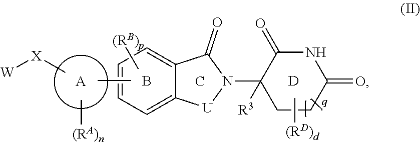
or a pharmaceutically acceptable salt, solvate, or stereoisomer thereof, wherein:
W is —N(R¹)₂, 3-to 12-membered heterocyclyl, or 5- to 10-membered heteroaryl, wherein the heterocyclyl or heteroaryl is optionally substituted with one or more R^{1b},
two R¹, together with the nitrogen atom to which they are attached, form 3- to 12-membered heterocyclyl or 5- to 10-membered heteroaryl, wherein the heterocyclyl or heteroaryl is optionally substituted with one or more R^{1b};
each R^{1b} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c} S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(—O) R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c} R⁴, —C(═O) R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
two vincinal R^{1b}, together with the intervening atoms, form C₆₋₁₀ aryl or 5- to 10-membered heteroaryl, wherein the aryl or heteroaryl is optionally substituted with one or more R^{u}; or
each R¹ is independently hydrogen, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —S(═O)₂R^{a}, —S(═O)₂OR^{b}, —S(═O)₂NR^{c}R^{d}, —C(═O)R^{a}, —C(—O)OR^{b}, or —C(—O)NR^{c}R^{d}, wherein the alkyl, alkylene, alkenyl, alkynyl, carbocyclyl, heterocyclyl, aryl, or heteroaryl is optionally substituted with one or more R^{1a},
each R^{1a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(—O)₂R^{a}, —S(═O)₂OR^{b}, —S(═O)₂NR^{c}R^{d}, —NR^{c}S(═O)₂R^{a}, —NR^{c} S(═O) R^{a}, —NR^{c}S(═O)₂OR^{b}, —NR^{c}S(═O)₂NR^{c}R^{d}, —NR^{b}C(═O)NR^{c}R^{d}, —NR^{b}C(—O) R^{a}, —NR^{b}C(—O)OR^{b}, —OS(═O)₂R^{a}, —OS(═O)₂OR^{b}, —OS(═O)₂NR^{c} R⁴, —OC(—O)R^{a}, —OC(═O)OR^{b}, —OC(═O)NR^{c} R⁴, —C(—O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
X is —[C(R²)₂]—ₘ, O, or NR^{X}; wherein when X is O or NR^{X}, then W is 3- to 12-membered heterocyclyl or 5- to 10-membered heteroaryl, wherein the heterocyclyl or heteroaryl is optionally substituted with one or more R^{1b};
each R² is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(—O)R^{a}, —S(═O)₂R^{a}, —S(═O)₂OR^{b}, —S(═O)₂NR^{c} R⁴, —NR^{c} S(—O)₂R^{a}, —NR^{c} S(═O) R^{a}, —NR^{c} S(═O)₂OR^{b}, —NR^{c}S(═O)₂NR^{c}R^{d}, —NR^{b}C(═O)NR^{c} R^{a}, —NR^{b}C(═O) R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c} R⁴, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two geminal R² together form an oxo; or
two germinal R², together with the carbon atom to which they are attached, form C₃₋₆ carbocyclyl or 3- to 6-membered heterocyclyl, wherein the carbocyclyl or heterocyclyl is optionally substituted with one or more R^{u};
m is an integer from 0 to 5;
R^{X} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
Ring A is 9- or 10-membered bicyclic fused ring system comprising at least one 5- or 6-membered heteoaryl;
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(—O)₂OR^{b}, —S(═O)₂NR^{c}R^{d}, —NR^{c} S(—O)₂R^{a}, —NR^{c} S(—O) R^{a}, —NR^{c} S(═O)₂OR^{b}, —NR^{c}S(═O)₂NR^{c}R^{d}, —NRbC(═O)NR^{c}R^{d}, —NR^{b}C(—O)R^{a}, —NRDC(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(—O)NR^{c} R⁴, —C(═O)R^{a}, —C(═O)OR^{b}, or —C(—O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
n is an integer from 0 to 10, as valency permits; or
two vincinal R^{A}, together with the intervening atoms, form C₃₋₁₂ carbocyclyl or 3- to 12-membered heterocyclyl, wherein the carbocyclyl or heterocyclyl is optionally substituted with one or more R^{u};
each R^{B} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O) R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(—O)OR^{b}, —OS(—O)₂R^{a}, —OS(═O)₂OR^{b}, —OS(═O)₂NR^{c}R^{d}, —OC(═O)R^{a}, —OC(═O)OR^{b}, —OC(═O)NR^{c} R⁴, —C(═O)R^{a}, —C(—O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
p is an integer from 0 to 3;
U is —C(R⁴)₂— or —C(═O)—;
each R⁴ is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}; or
two R⁴, together with the carbon atom to which they are attached, form C₃₋₆ carbocyclyl or 3- to 6-membered heterocyclyl, wherein the carbocyclyl or heterocyclyl is optionally substituted with one or more R^{u};
each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 4;
R³ is hydrogen, deuterium, C₁₋₆ haloalkyl, or C₁₋₆ alkyl; and
q is an integer from 0 to 2;
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(—O)R^{a}, —S(═O)₂R^{a}, —S(═O)₂OR^{b}, —S(═O)₂NR^{c}R^{d}, —NR^{c}S(═O)₂R^{a}, —NR^{c}S(═O) R^{a}, —NR^{c} S(═O)₂OR^{b}, —NR^{c}S(═O)₂NR^{c}R^{d}, —NRDC(═O)NR^{c}R^{d}, —NR^{b}C(═O) R^{a}, —NRDC(═O)OR^{b}, —OS(═O)₂R^{a}, —OS(═O)₂OR^{b}, —OS(═O)₂NR^{c} R⁴, —OC(═O)R^{a}, —OC(═O)OR^{b}, —OC(—O)NR^{c}R^{d}, —C(═O) R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, C₆ aryl, and 5- to 6-membered heteroaryl; or
two R^{u}, together with the one or more intervening atoms, form C₆₋₁₀ aryl, 5- to 10-membered heteroaryl, C₃₋₁₂ carbocyclyl, or 3- to 12-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl or 5- to 10-membered heteroaryl, wherein the heterocyclyl or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₃₋₆ carbocyclyl, and 3- to 6-membered heterocyclyl;
wherein each of R^{a}, R^{b}, R^{c}, and R^{d} is independently and optionally substituted with one or more R^{z};
each R^{z} is independently oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, C₆ aryl, or 5- to 6-membered heteroaryl,
provided that:
i) when Ring A is 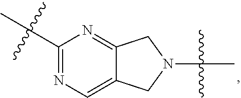 [SMILES: *c1ncc2c(n1)CN(*)C2]
then m is not 0;
ii) when each R¹ is independently hydrogen, C₁₋₆ alkyl, C₃₋₁₂ carbocyclyl, or —C(═O) (C₁₋₆ alkyl), then 1) m is not 0; and 2) two geminal R² do not together form an oxo; and
iii) the compound is not

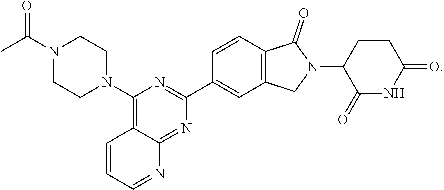 [SMILES: CC(=O)N1CCN(c2nc(-c3ccc4c(c3)CN(C3CCC(=O)NC3=O)C4=O)nc3ncccc23)CC1]

2. The compound of claim 1, wherein the compound is a compound of Formula II-1 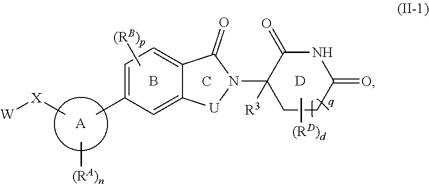
or a pharmaceutically acceptable salt, solvate, or stereoisomer thereof.

3. The compound of claim 1 or 2, wherein X is —[C(R²)₂]—ₘ.

4. The compound any one of claims 1-3, wherein W is —N(R¹)₂.

5. The compound of claim 1, wherein the compound is a compound of Formula II-2 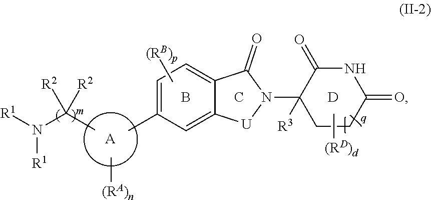
or a pharmaceutically acceptable salt, solvate, or stereoisomer thereof.

6. The compound of any one of claims 1-5, wherein Ring A is 9-membered bicyclic heteroaryl comprising 1 to 4 nitrogen atoms.

7. The compound of claim 6, wherein Ring A is imidazo[1,5-a]pyridinyl, 1H-pyrrolo[2,3-b]pyridinyl, [1,2,4]triazolo[4,3-a]pyridinyl, imidazo[1,2-a]pyridinyl, indolyl, benzo[d]imidazolyl, indazolyl, benzo[d]isoxazolyl, benzo[d]oxazolyl, benzo[d]isothiazolyl, benzo[d]thiazolyl, benzo[b]thiophenyl, or benzofuranyl.

8. The compound of any one of claims 1-5, wherein 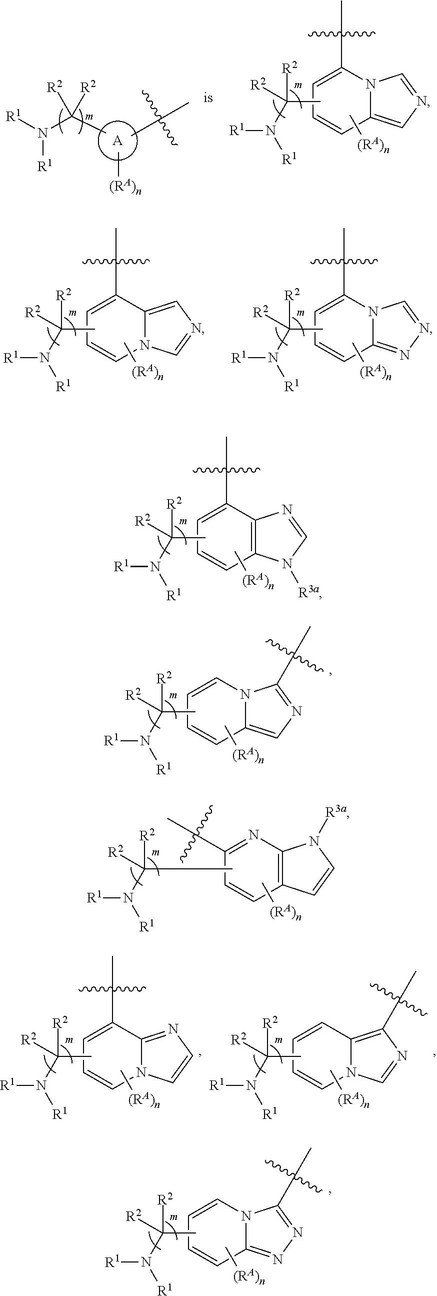 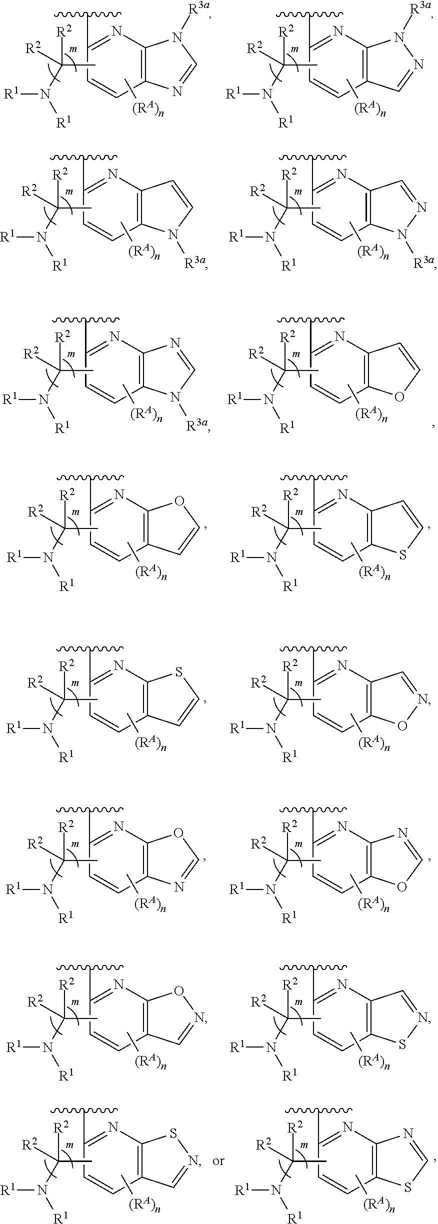
wherein:
R^{3a} is hydrogen, C₁₋₆ alkyl, C₁₋₆ heteroalkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —(C₁₋₃ alkylene)-(C₃₋₆ carbocyclyl), —(C₁₋₃ alkylene)-(3- to 6-membered heterocyclyl), —(C₁₋₃ alkylene)-(C₆ aryl), —(C₁₋₃ alkylene)-(5- to 6-membered heteroaryl), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkylene, alkenyl, alkynyl, carbocyclyl, heterocyclyl, aryl, or heteroaryl is optionally substituted with one or more R^{u}.

9. The compound of claim 8, wherein

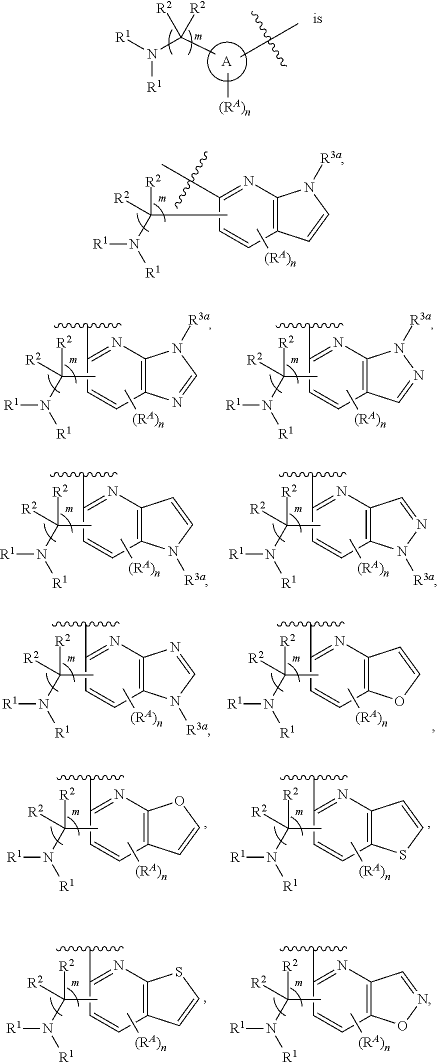

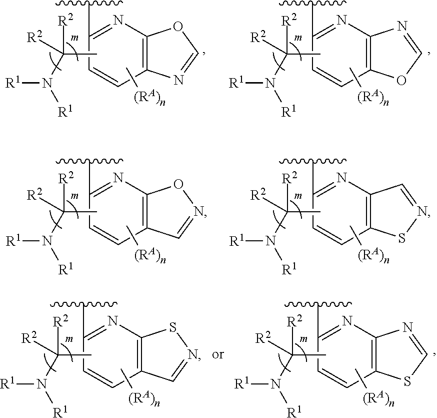

10. The compound of claim 8 or 9, wherein R^{3a} is hydrogen, C₁₋₆ alkyl, C₁₋₆ heteroalkyl, C₂-6 alkynyl, C₃₋₁₂ carbocyclyl, 3- to 12-membered heterocyclyl, C₆₋₁₀ aryl, 5- to 10-membered heteroaryl, or —(C₁₋₃ alkylene)-(C₆ aryl), wherein the alkyl, alkylne, alkynyl, carbocyclyl, heterocyclyl, aryl, or heteroaryl is optionally substituted with one or more R^{u}.

11. The compound of any one of claims 1-5, wherein Ring A is 10-membered bicyclic heteroaryl comprising 1 to 3 nitrogen atoms.

12. The compound of claim 10, wherein

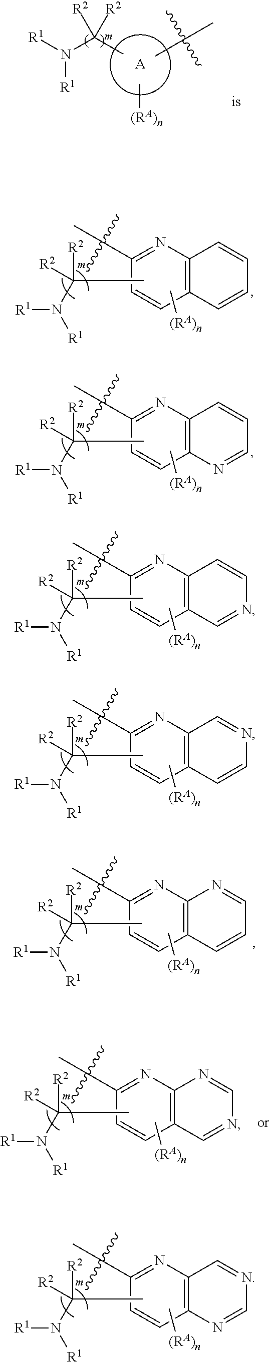

13. The compound of any one of claims 1-5, wherein Ring A is 9- or 10-membered bicyclic fused ring system comprising one 5- or 6-membered heteoaryl and one C₅₋₆ carbocyclyl.

14. The compound of claim 13, wherein

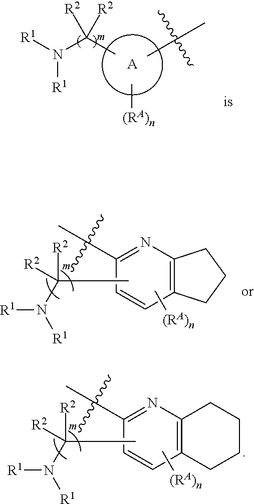

15. The compound of any one of claims 1-14, wherein each R¹ is independently hydrogen, C₁₋₆ alkyl, —(C₁₋₆ alkylene)-(C₆₋₁₀ aryl), or —(C₁₋₆ alkylene)-(5- to 10-membered heteroaryl), wherein the alkyl, alkylene, aryl, or heteroaryl is optionally substituted with one or more R^{1a}.

16. The compound of claim 15, wherein each R^{1a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

17. The compound of claim 15, wherein each R^{1a} is independently halogen, C₁₋₆ alkyl, C₆-10 aryl, or 5- to 10-membered heteroaryl, wherein the alkyl, aryl, or heteroaryl is optionally substituted with one or more R^{u}.

18. The compound of any one of claims 1-14, wherein two R¹, together with the nitrogen atom to which they are attached, form 5- or 10-membered heterocyclyl optionally substituted with one or more R^{1b}.

19. The compound of any one of claims 1-14, wherein two R¹, together with the nitrogen atom to which they are attached, form 5- to 10-membered heteroaryl optionally substituted with one or more R^{1b}.

20. The compound of claim 18 or 19, wherein each R^{1b} is independently oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, C₆ aryl, 5- to 6-membered heteroaryl, or —S(—O)₂R^{a}, wherein the alkyl, alkoxy, alkylamino, carbocyclyl, heterocyclyl, aryl, or heteroaryl is optionally substituted with one or more R^{u}.

21. The compound of claim 18 or 19, wherein each R^{1b} is independently oxo, halogen, —CN, —OH, C₁₋₆ alkyl, C₁₋₆ alkoxy, C₁₋₆ alkylamino, C₆ aryl, 5- to 6-membered heteroaryl, or —S(═O)₂R^{a}, wherein the alkyl, alkoxy, alkylamino, aryl, or heteroaryl is optionally substituted with one or more R^{u}.

22. The compound of any one of claims 1-21, wherein each R² is independently hydrogen or C₁₋₆ alkyl.

23. The compound of any one of claims 1-21, wherein each R² is hydrogen.

24. The compound of any one of claims 1-21, wherein two R² together form an oxo.

25. The compound of any one of claims 1-24, wherein m is 1.

26. The compound of any one of claims 1-25, wherein each R^{A} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, C₆ aryl, 5- to 6-membered heteroaryl, or —NR^{c}S(═O)R^{a}, wherein the alkyl, alkoxy, alkylamino, carbocyclyl, heterocyclyl, aryl, or heteroaryl is optionally substituted with one or more R^{u}.

27. The compound of any one of claims 1-25, wherein each R^{A} is independently oxo, halogen, —NH₂, C₁₋₆ alkyl, C₁₋₆ alkoxy, C₁₋₆ alkylamino, C₂₋₆ alkynyl, C₃₋₆ carbocyclyl, 3- to 6-membered heterocyclyl, C₆ aryl, 5- to 6-membered heteroaryl, or —NR^{c}S(═O)R^{a}, wherein the alkyl, alkoxy, alkylamino, alkynyl, carbocyclyl, heterocyclyl, aryl, or heteroaryl is optionally substituted with one or more R^{u}.

28. The compound of any one of claims 1-27, wherein n is 0, 1, or 2.

29. The compound of any one of claims 1-28, wherein each R^{B} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

30. The compound of any one of claims 1-28, wherein each R^{B} is independently halogen, C₁₋₆ alkyl, or C₁₋₆ alkoxy.

31. The compound of any one of claims 1-30, wherein p is 0 or 1.

32. The compound of any one of claims 1-31, wherein U is —C(R⁴)₂—, and each R⁴ is independently hydrogen or C₁₋₆ alkyl.

33. The compound of any one of claims 1-32, wherein each R^{D} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

34. The compound of any one of claims 1-33, wherein d is 0.

35. The compound of any one of claims 1-34, wherein R³ is hydrogen.

36. The compound of any one of claims 1-35, wherein q is 1.

37. The compound of claim 1, wherein the compound is a compound of Formula II-2 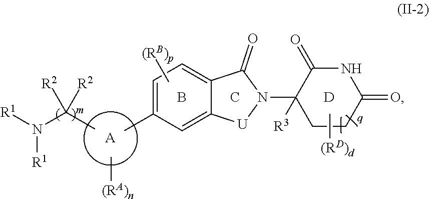
or a pharmaceutically acceptable salt, solvate, or stereoisomer thereof, wherein:
two R¹, together with the nitrogen atom to which they are attached, form 3- to 12-membered heterocyclyl optionally substituted with one or more R^{1b},
each R^{1b} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c} S(═O)₂R^{a}, —NR^{c} S(═O) R^{a}, —NR^{c}S(═O)₂OR^{b}, —NR^{c}S(═O)₂NR^{c}R^{d}, —NR^{b}C(═O)NR^{c}R^{d}, —NR^{b}C(—O) R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(—O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
two vincinal R^{1b}, together with the intervening atoms, form C₆ aryl or 5- to 6-membered heteroaryl, wherein the aryl or heteroaryl is optionally substituted with one or more R^{u};
each R² is hydrogen;
m is 1;
Ring A is 9- or 10-membered bicyclic fused heteroaryl or 9- or 10-membered bicyclic fused ring system comprising one 5- or 6-membered heteoaryl and one C₅₋₆ carbocyclyl;
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c} S(═O)₂R^{a}, —NR^{c} S(═O) R^{a}, —NR^{c} S(═O)₂OR^{b}, —NR^{c}S(═O)₂NR^{c} R⁴, —NRDC(═O)NR^{c}R^{d}, —NRDC(—O)R^{a}, —NRDC(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c} R⁴, —C(═O) R^{a}, —C(═O)OR^{b}, or — C(—O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
n is an integer from 0 to 2;
each R^{B} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c} S(═O)₂R^{a}, —NR^{c} S(—O) R^{a}, —NR^{c} S(═O)₂OR^{b}, —NR^{c} S(—O)₂NR^{c} R^{a}, —NR^{b}C(═O)NR^{c} R⁴, —NR^{b}C(═O)R^{a}, —NRDC(═O)OR^{b}, —OS(═O)₂R^{a}, —OS(═O)₂OR^{b}, —OS(═O)₂NR^{c}R^{d}, —OC(—O)R^{a}, —OC(═O)OR^{b}, —OC(═O)NR^{c}R^{d}, —C(═O) R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
p is an integer from 0 to 3;
U is —CH₂—;
each R^{D} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u};
d is an integer selected from 0 to 4;
R³ is hydrogen, deuterium, C₁₋₆ haloalkyl, or C₁₋₆ alkyl; and
q is 1.

38. The compound of claim 1, wherein the compound is selected from the compounds in Table 1 and pharmaceutically acceptable salts thereof.

39. A pharmaceutical composition comprising the compound of any one of claims 1-38, and a pharmaceutically acceptable excipient.

40. A method of degrading an IKZF2 protein in a subject or biological sample comprising administering the compound of any one of claims 1-38 to the subject or contacting the biological sample with the compound of any one of claims 1-38.

41. Use of the compound of any one of claims 1-38 in the manufacture of a medicament for degrading an IKZF2 protein in a subject or biological sample.

42. The compound of any one of claims 1-38 for use in degrading an IKZF2 protein in a subject or biological sample.

43. A method of treating or preventing a disease or disorder a subject in need thereof, comprising administering to the subject the compound of any one of claims 1-38.

44. Use of the compound of any one of claims 1-38 in the manufacture of a medicament for treating or preventing a disease or disorder in a subject in need thereof.

45. The compound of any one of claims 1-38 for use in treating or preventing a disease or disorder in a subject in need thereof.

46. The method, use, or compound for use of any one of claims 43-45, wherein the disease or disorder is an IKZF2-mediated disease or disorder.

47. The method, use, or compound of any one of claims 43-45, wherein the disease or disorder is T cell leukemia, T cell lymphoma, Hodgkin's lymphoma, non-Hodgkin's lymphoma, myeloid leukemia, non-small cell lung cancer (NSCLC), melanoma, triple-negative breast cancer (TNBC), nasopharyngeal cancer (NPC), microsatellite stable colorectal cancer (mssCRC), thymoma, carcinoid, or gastrointestinal stromal tumor (GIST).
